(19) United States
(12) Patent Application Publication
(10) Pub. No.: US 2016/0075653 A1
(43) Pub. Date: Mar. 17, 2016

(54) NEMATICIDAL N-(PHENYLCYCLOALKYL)CARBOXAMIDES AND N-(PHENYLCYCLOALKYL)THIOCARBOXAMIDES

(71) Applicant: BAYER CROPSCIENCE AKTIENGESELLSCHAFT, Monheim am Rhein (DE)

(72) Inventors: Anne DECOR, Langenfeld (DE); Joerg GREUL, Leverkusen (DE); Eike Kevin HEILMANN, Duesseldorf (DE); Hans-Georg SCHWARZ, Dorsten (DE); Ernst Rudolf GESING, Erkrath (DE); Jens FRACKENPOHL, Frankfurt (DE); Hans-Ludwig ELBE, Wuppertal (DE); Welf Burkhard WIESE, Langenfeld (DE); Daniela PORTZ, Vettweiss (DE); Kerstin ILG, Koeln (DE); Olga MALSAM, Roesrath (DE); Peter LOESEL, Leverkusen (DE); Peter LUEMMEN, Idstein (DE); Ulrich GOERGENS, Ratingen (DE); Pierre-Yves COQUERON, Lyon (DE); Arianna MARTELLETTI, Sulzbach im Taunus (DE); Philippe DESBORDES, Lyon (FR); Stephanie GARY, Champagne au Mont d'or (FR); Isabelle CHRISTIAN, Lyon (FR); Claudia WELZ, Duesseldorf (DE)

(21) Appl. No.: 14/787,530

(22) PCT Filed: Apr. 28, 2014
(86) PCT No.: PCT/EP2014/058539

§ 371 Date: Oct. 28, 2015

(30) Foreign Application Priority Data
Apr. 30, 2013 (EP) 13165996.3
Nov. 22, 2013 (EP) 13194048.8

(51) Int. Cl. C07D 213/81; C07D 213/82; C07C 233/66; C07D 231/14

(52) U.S. Cl. CPC C07D 213/81; C07D 231/14; C07D 213/82; C07C 233/66; C07C 2101/04; C07C 2101/02

(57) ABSTRACT

The present invention relates to the use of N-(Phenylcycloalkyl)carboxamides and N-(Phenylcycloalkyl)thiocarboxamides for the control of nematodes in agriculture and as anthelmintic agents against endoparasites in animals and humans, compositions containing such compounds and methods for the control of nematodes and helminths and furthermore to novel N-(Phenylcycloalkyl)carboxamides and N-(Phenylcycloalkyl)thiocarboxamides, processes and intermediate compounds for their preparation, their use as nematicides, compositions containing such compounds and methods for the control of nematodes.

The present invention relates to the use of N-(Phenylcycloalkyl)carboxamides and N-(Phenylcycloalkyl)thiocarboxamides for the control of nematodes in agriculture and as anthelmintic agents against endoparasites in animals and humans, compositions containing such compounds and methods for the control of nematodes and helminths.

The present invention further relates to novel N-(Phenylcycloalkyl)carboxamides and N-(Phenylcycloalkyl)thiocarboxamides, processes and intermediate compounds for their preparation, their use as nematicides, compositions containing such compounds and methods for the control of nematodes.

Nematodes cause a substantial loss in agricultural product including food and industrial crops and are controlled with chemical compounds having nematicidal activity. To be useful in agriculture these compounds should have a high activity, a broad spectrum activity against different strains of nematodes and should not be toxic to non-target organisms.

The occurrence of resistances against all commercially available anthelmintics seems to be a growing problem in the area of veterinary medicine. Therefore, endoparasiticides with new molecular modes of actions are urgently desired. The new active ingredients should perform with excellent efficacy against a broad spectrum of helminths and nematodes without any adverse toxic effects to the treated animal. Endoparasiticides are pharmaceuticals for the control or suppression of endoparasites in animals or humans.

N-(Phenylcyclopropyl)carboxamides and N-(Phenylcyclobutyl)carboxamides, their use and methods for their preparation are disclosed in WO 2013/120940 A2 and WO 2013/143811 A1.

In WO 1998/03495, certain fungicidal N-(Phenylcyclohexyl)carboxamide derivatives are generically embraced in a broad disclosure of numerous compounds of the following formula:

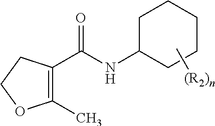

wherein R₂ can represent a substituted aryl or a substituted aralkyl and n can be 1. However, this document is silent about a nematicidal efficacy of the disclosed compounds.

In WO 2007/134799 certain fungicidal N-(Phenylcyclopropyl) (thio)carboxamide derivatives are generically embraced in a broad disclosure of numerous compounds of the following formula:

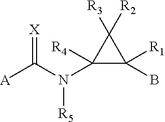

wherein A can represent phenyl, a 5- or 6-membered heterocyclic ring, X can be an oxygen or sulphur atom, R₁, R₂, R₃ and R₄ can represent hydrogen, halogen, C₁-C₄-alkyl, cyano or nitro, B can represent substituted phenyl and R₅ can represent various substituents, e.g. hydrogen or C₁-C₄-alkyl. However, this document does not disclose compounds with nematicidal activity.

In WO 2005/103006, WO 2006/122952, WO 2005/103004, WO 2006/122955, and EP 2 289 880 certain fungicidal N-(2-Pyridylcycloalkyl) carboxamide derivatives are generically embraced in a broad disclosure of numerous compounds of the following formula:

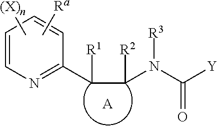

wherein Y can represent phenyl or a heterocyclic ring, A can represent C₃-C₇-cycloalkyl, R¹ and R² can represent various substituents, e.g. hydrogen or C₁-C₆-alkyl, R³ can represent various substituents, e.g. hydrogen, C₁-C₆-alkyl or C₃-C₇-cycloalkyl, R^{a} represents C₁-C₆-halogenoalkyl and X can represent hydrogen, halogen, C₁-C₆-alkyl or C₁-C₆-halogenoalkyl. However, these documents do not disclose compounds wherein the 2-pyridine moiety can be replaced by a phenyl group.

In WO 2010/094666 A2, certain fungicidal N-(phenylcycloalkyl)-carboxamides and thiocarboxamides are generically embraced in a broad disclosure of numerous compounds of the following formula:

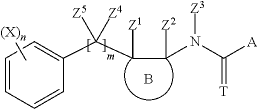

wherein A can represent a 5-membered heterocyclic ring, T can be oxygen or sulfur, Z¹, Z², Z⁴ and Z⁵ can represent various substituents, e.g. hydrogen and C₁-C₆-alkyl, Z³ can represent C₃-C₇-cycloalkyl, m can represent 0 or 1, X can represent various substituents, e.g. hydrogen and C₁-C₆-alkyl. However, this document does not claim nematicidal use for the disclosed compounds.

In WO2011/151369 certain [(het)arylethyl)]pyrazolecarboxamides or thio-carboxamides are generically embraced in a broad disclosure of numerous compounds of the following formula:

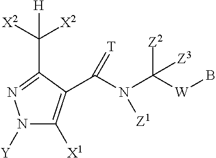

wherein B can represent a substituted or unsubstituted ring and W can, amongst other groups, represent CZ⁴Z⁵, in particular Z² or Z³ and Z⁴ or Z⁵ can, together with the carbon atoms to which they are linked, form a 3-, 4-, 5-, 6- or 7-membered saturated carbocycle. However, this document does not disclose a nematicidal use for the disclosed compounds.

Modern crop protection compositions have to meet many demands, for example in relation to the level, duration and breadth of their action and possible use. Questions of toxicity and of combinability with other active ingredients or formulation auxiliaries play a role, as does the question of the expense that the synthesis of an active ingredient requires. In addition, resistances can occur. For all these reasons, the search for novel crop protection compositions cannot be considered to be complete, and there is a constant need for novel compounds having properties which, compared to the known compounds, are improved at least in relation to individual aspects.

It was an object of the present invention to provide compounds which widen the spectrum of the pesticides in various respects.

This object, and further objects which are not stated explicitly but can be discerned or derived from the connections discussed herein, are achieved by the use of compounds of formula (I)

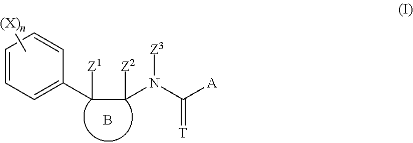

wherein
A represents phenyl of formula A^{a}

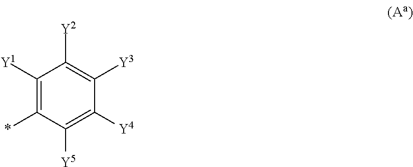

wherein
* indicates the bond which connects A^{a} to the C=T moiety of the compounds of formula (I),
Y¹ represents halogen, nitro, SH, SF₅, CHO, OCHO, NHCHO, cyano, C₁-C₈-alkyl, C₁-C₈-halogenoalkyl having 1 to 5 halogen atoms, C₂-C₈-alkenyl, C₂-C₈-alkynyl, C₃-C₆-cycloalkyl, C₃-C₆-halogenocycloalkyl having 1 to 9 halogen atoms, C₁-C₈-alkylsulfanyl, —C₁-C₈-halogenoalkylsulfanyl having 1 to 5 halogen atoms, C₁-C₈-alkoxy, C₁-C₈-halogenoalkoxy having 1 to 5 halogen atoms, C₁-C₈-alkoxy-C₁-C₈-alkyl, C₂-C₈-alkenyloxy-C₁-C₈-alkyl, C₃-C₈-alkynyloxy-C₁-C₈-alkyl, C₂-C₈-alkenyloxy, C₃-C₈-alkynyloxy, C₁-C₈-alkoxycarbonyl, C₁-C₈-halogenoalkoxycarbonyl having 1 to 5 halogen atoms, C₁-C₈-alkylcarbonyloxy, C₁-C₈-halogenoalkylcarbonyloxy having 1 to 5 halogen atoms, C₁-C₈-alkylsulfinyl, C₁-C₈-halogenoalkylsulfinyl having 1 to 5 halogen atoms, C₁-C₈-alkylsulfonyl, C₁-C₈-halogenoalkylsulfonyl having 1 to 5 halogen atoms, C₁-C₈-alkylsulfonamide, substituted or unsubstituted tri-(C₁-C₈)-alkylsilyl, substituted or unsubstituted aryl and substituted or unsubstituted aryloxy;
Y², Y³, Y⁴ and Y⁵ independently from each other represent hydrogen or Y¹; or
A represents a carbo-linked heterocyclyl group optionally substituted by 1 to five groups R, wherein
R independently from each other R represents hydrogen, halogen, nitro, cyano, hydroxy, amino, sulfanyl, pentafluoro-λ⁶-sulfanyl, substituted or unsubstituted (C₁-C₈-alkoxyimino)-C₁-C₈-alkyl, substituted or unsubstituted (benzyloxyimino)-C₁-C₈-alkyl, substituted or unsubstituted C₁-C₈-alkyl, C₁-C₈-halogenoalkyl having 1 to 5 halogen atoms, substituted or unsubstituted C₂-C₈-alkenyl, C₂-C₈-halogenoalkenyl having 1 to 5 halogen atoms, substituted or unsubstituted C₂-C₈-alkynyl, C₂-C₈-halogenoalkynyl having 1 to 5 halogen atoms, substituted or unsubstituted C₁-C₈-alkoxy, C₁-C₈-halogenoalkoxy having 1 to 5 halogen atoms, substituted or unsubstituted C₁-C₈-alkylsulfanyl, C₁-C₈-halogenoalkylsulfanyl having 1 to 5 halogen atoms, substituted or unsubstituted C₁-C₈-alkylsulfinyl, C₁-C₈-halogenoalkylsulfinyl having 1 to 5 halogen atoms, substituted or unsubstituted C₁-C₈-alkylsulfonyl, C₁-C₈-halogenoalkylsulfonyl having 1 to 5 halogen atoms, substituted or unsubstituted C₁-C₈-alkylamino, substituted or unsubstituted di-C₁-C₈-alkylamino, substituted or unsubstituted C₂-C₈-alkenyloxy, substituted or unsubstituted C₃-C₈-alkynyloxy, substituted or unsubstituted C₃-C₇-cycloalkyl, C₃-C₇-halogenocycloalkyl having 1 to 5 halogen atoms, substituted or unsubstituted tri-(C₁-C₈-alkyl)-silyl, substituted or unsubstituted C₁-C₈-alkylcarbonyl, C₁-C₈-halogenoalkylcarbonyl having 1 to 5 halogen atoms, substituted or unsubstituted C₁-C₈-alkoxycarbonyl, C₁-C₈-halogenoalkoxycarbonyl having 1 to 5 halogen atoms, substituted or unsubstituted C₁-C₈-alkylcarbamoyl, substituted or unsubstituted di-(C₁-C₈)-alkylcarbamoyl, phenoxy, phenylsulfanyl, phenylamino, benzyloxy, benzylsulfanyl, or benzylamino,
T represents oxygen or sulphur,
B represents a 3-, 4-, 5-, 6- or 7-membered non-aromatic carbocycle optionally substituted by substituents selected from the group consisting of one to four C₁-C₈-alkyl groups and one to four halogen atoms,
n represents 0, 1, 2, 3, 4 or 5,
X independently from each other X represents halogen, nitro, cyano, isonitrile, hydroxy, amino, sulfanyl, pentafluoro-λ⁶-sulfanyl, formyl, formyloxy, formylamino, substituted or unsubstituted (hydroxyimino)-C₁-C₈-alkyl, substituted or unsubstituted (C₁-C₈-alkoxyimino)-C₁-C₈-alkyl, substituted or unsubstituted (C₂-C₈-alkenyloxyimino)-C₁-C₈-alkyl, substituted or unsubstituted (C₃-C₈-alkynyloxyimino)-C₁-C₈-alkyl, substituted or unsubstituted (benzyloxyimino)-C₁-C₈-alkyl, carboxy, carbamoyl, thiocarbamoyl, N-hydroxycarbamoyl, carbamate, substituted or unsubstituted C₁-C₈-alkyl, C₁-C₈-halogenoalkyl having 1 to 9 halogen atoms, substituted or unsubstituted C₂-C₈-alkenyl, C₂-C₈-halogenoalkenyl having 1 to 9 halogen atoms, substituted or unsubstituted C₂-C₈-alkynyl, C₂-C₈-halogenoalkynyl having 1 to 9 halogen atoms, substituted or unsubstituted C₁-C₈-alkoxy, C₁-C₈-halogenoalkoxy having 1 to 9 halogen atoms, substituted or unsubstituted C₁-C₈-alkylsulfanyl, C₁-C₈-halogenoalkylsulfanyl having 1 to 9 halogen atoms, substituted or unsubstituted C₁-C₈-alkylsulfinyl, C₁-C₈-halogenoalkylsulfinyl having 1 to 9 halogen atoms, substituted or unsubstituted C₁-C₈-alkylsulfonyl, C₁-C₈-halogenoalkylsulfonyl having 1 to 9 halogen atoms, substituted or unsubstituted C₁-C₈-alkylamino, substituted or unsubstituted di-(C₁-C₈-alkyl)-amino, substituted or unsubstituted C₂-C₈-alkenyloxy, C₂-C₈-halogenoalkenyloxy having 1 to 9 halogen atoms, substituted or unsubstituted C₃-C₈-alkynyloxy, C₃-C₈-halogenoalkynyloxy having 1 to 9 halogen atoms, substituted or unsubstituted C₃-C₇-cycloalkyl, C₃-C₇-halogenocycloalkyl having 1 to 9 halogen atoms, substituted or unsubstituted C₃-C₇-cycloalkyloxy, substituted or unsubstituted C₄-C₇-cycloalkenyl, C₄-C₇-halogenocycloalkenyl having 1 to 9 halogen atoms, substituted or unsubstituted (C₃-C₇-cycloalkyl)-C₁-C₈-alkyl, substituted or unsubstituted (C₃-C₇-cycloalkyl)-C₂-C₈-alkenyl, substituted or unsubstituted (C₃-C₇-cycloalkyl)-C₂-C₈-alkynyl, substituted or unsubstituted tri-(C₁-C₈-alkyl)-silyl, substituted or unsubstituted tri-(C₁-C₈-alkyl)-silyl-C₁-C₈-alkyl, substituted or unsubstituted C₁-C₈-alkylcarbonyl, C₁-C₈-halogenoalkylcarbonyl having 1 to 9 halogen atoms, substituted or unsubstituted C₁-C₈-alkylcarbonyloxy, C₁-C₈-halogenoalkylcarbonyloxy having 1 to 9 halogen atoms, substituted or unsubstituted C₁-C₈-alkylcarbonylamino, C₁-C₈-halogenoalkylcarbonylamino having 1 to 9 halogen atoms, substituted or unsubstituted C₁-C₈-alkoxycarbonyl, C₁-C₈-halogenoalkoxycarbonyl having 1 to 9 halogen atoms, substituted or unsubstituted C₁-C₈-alkyloxycarbonyloxy, C₁-C₈-halogenoalkoxycarbonyloxy having 1 to 9 halogen atoms, substituted or unsubstituted C₁-C₈-alkylcarbamoyl, substituted or unsubstituted di-C₁-C₈-alkylcarbamoyl, substituted or unsubstituted C₁-C₈-alkylaminocarbonyloxy, substituted or unsubstituted di-C₁-C₈-alkylaminocarbonyloxy, substituted or unsubstituted N—(C₁-C₈-alkyl)-hydroxycarbamoyl, substituted or unsubstituted C₁-C₈-alkoxycarbamoyl, substituted or unsubstituted N—(C₁-C₈-alkyl)-C₁-C₈-alkoxycarbamoyl, aryl optionally substituted by 1 to 6 groups Q which can be the same or different, aryl-C₁-C₈-alkyl optionally substituted by 1 to 6 groups Q which can be the same or different, aryl-C₂-C₈-alkenyl optionally substituted by 1 to 6 groups Q which can be the same or different, aryl-C₂-C₈-alkynyl optionally substituted by 1 to 6 groups Q which can be the same or different, aryloxy optionally substituted by 1 to 6 groups Q which can be the same or different, arylsulfanyl optionally substituted by 1 to 6 groups Q which can be the same or different, arylamino optionally substituted by 1 to 6 groups Q which can be the same or different, aryl-C₁-C₈-alkyloxy optionally substituted by 1 to 6 groups Q which can be the same or different, aryl-C₁-C₈-alkylsulfanyl optionally substituted by 1 to 6 groups Q which can be the same or different, aryl-C₁-C₈-alkylamino optionally substituted by 1 to 6 groups Q which can be the same or different, pyridinyl which can be substituted by 1 to 4 groups Q, pyridinyloxy which is optionally substituted by 1 to 4 groups Q, or
two substituents X together with the carbon atoms to which they are attached form a 5- or 6-membered, saturated carbocycle or saturated heterocycle, which is optionally substituted by 1 to 4 groups Q which can be the same or different,
Z¹ and Z² independently represent hydrogen, halogen, cyano, substituted or unsubstituted C₁-C₈-alkyl, C₁-C₈-halogenoalkyl having 1 to 5 halogen atoms, substituted or unsubstituted C₁-C₈-alkoxy, substituted or unsubstituted C₁-C₈-alkylsulfanyl, or substituted or unsubstituted C₁-C₈-alkoxycarbonyl,
Z³ represents hydrogen, cyano, C₁-C₈-alkoxy, unsubstituted C₃-C₇-cycloalkyl or C₃-C₇-cycloalkyl substituted by 1 to 10 substituents that can be the same or different, selected from the list consisting of halogen, cyano, C₁-C₈-alkyl, C₁-C₈-halogenoalkyl comprising 1 to 9 halogen atoms, C₁-C₈-alkoxy, C₁-C₈-halogenoalkoxy comprising 1 to 9 halogen atoms, C₁-C₈-alkoxycarbonyl, C₁-C₈-halogenoalkoxycarbonyl comprising 1 to 9 halogen atoms, C₁-C₈-alkylaminocarbonyl and di-(C₁-C₈-alkyl)-amino,
Q represents halogen, cyano, nitro, substituted or unsubstituted C₁-C₈-alkyl, C₁-C₈-halogenoalkyl having 1 to 9 halogen atoms, substituted or unsubstituted C₁-C₈-alkoxy, C₁-C₈-halogenoalkoxy having 1 to 9 halogen atoms, substituted or unsubstituted C₁-C₈-alkylsulfanyl, C₁-C₈-halogenoalkylsulfanyl having 1 to 9 halogen atoms, substituted or unsubstituted tri-(C₁-C₈-alkyl)-silyl, substituted or unsubstituted tri-(C₁-C₈-alkyl)-silyl-C₁-C₈-alkyl, substituted or unsubstituted C₁-C₈-alkoxyimino-C₁-C₈-alkyl, substituted or unsubstituted (benzyloxyimino)-C₁-C₈-alkyl,
and N-oxides, metallic complexes, metalloidic complexes and optically active isomers of the compounds of formula (I),
for controlling nematodes.

In the above definitions, unless stated otherwise,

halogen means fluorine, bromine, chlorine, iodine,
carboxy means —C(═O)OH,
carbonyl means —C(═O)—,
carbamoyl means —C(═O)NH₂,
alkylcarbamoyl means —C(═O)NHalkyl
dialkylcarbamoyl means —C(═O)N(alkyl)₂
N-hydroxycarbamoyl means —C(═O)NHOH,
SO represents a sulfoxyde group,
SO₂ represents a sulfone group,
an alkyl group, an alkenyl group and an alkynyl group as well as moieties containing these terms, can be linear or branched.

The term “aryl”, also in terms like arylalkyl, arylalkenyl, arylalkynyl, aryloxy means phenyl or naphthyl, wherein phenyl is optionally substituted by 1 to 5 groups Q, and naphtyl is optionally substituted by 1 to 6 groups Q.

The term “heterocyclyl” means a saturated or unsaturated 4-, 5-, 6-, 7-, 8-, 9-, or 10-membered ring comprising 1 to 4 heteroatoms selected from the list consisting of oxygen (O), nitrogen (N), and sulphur (S).

Heteroatom means an atom selected from the group consisting of O, N, and S.

Unless indicated otherwise, if more than one halogen atom is present in a halogenated radical, like e.g. halogenoalkyl, halogenoalkoxy, those halogen atoms can be the same or different.

Unless indicated otherwise, a group or a substituent which is substituted according to the invention can be substituted by one or more of the following groups or atoms: halogen, nitro, hydroxy, cyano, amino, sulfanyl, pentafluoro-λ⁶-sulfanyl, formyl, formyloxy, formylamino, carbamoyl, N-hydroxycarbamoyl, carbamate, hydroxyimino-C₁-C₆-alkyl, C₁-C₈-alkyl, tri-(C₁-C₈-alkyl)-silyl-C₁-C₈-alkyl, C₁-C₈-cycloalkyl, tri-(C₁-C₈-alkyl)-silyl-C₁-C₈-cycloalkyl, C₁-C₈-halogenoalkyl having 1 to 5 halogen atoms, C₃-C₈-halogenocycloalkyl having 1 to 5 halogen atoms, C₂-C₈-alkenyl, C₂-C₈-alkynyl, C₂-C₈-alkenyloxy, C₃-C₈-alkynyloxy, C₁-C₈-alkylamino, di-(C₁-C₈-alkyl)-amino, C₁-C₈-alkoxy, C₁-C₈-halogenoalkoxy having 1 to 5 halogen atoms, C₁-C₈-alkylsulfanyl, C₁-C₈-halogenoalkylsulfanyl having 1 to 5 halogen atoms, C₂-C₈-alkenyloxy, C₂-C₈-halogenoalkenyloxy having 1 to 5 halogen atoms, C₃-C₈-alkynyloxy, C₃-C₈-halogenoalkynyloxy having 1 to 5 halogen atoms, C₁-C₈-alkylcarbonyl, C₁-C₈-halogenoalkylcarbonyl having 1 to 5 halogen atoms, C₁-C₈-alkylcarbamoyl, di-(C₁-C₈-alkyl)-carbamoyl, N—C₁-C₈-alkyloxycarbamoyl, C₁-C₈-alkoxycarbamoyl, N—C₁-C₈-alkyl-C₁-C₈-alkoxycarbamoyl, C₁-C₈-alkoxycarbonyl, C₁-C₈-halogenoalkoxycarbonyl having 1 to 5 halogen atoms, C₁-C₈-alkylcarbonyloxy, C₁-C₈-halogenoalkylcarbonyloxy having 1 to 5 halogen atoms, C₁-C₈-alkylcarbonylamino, C₁-C₈-halogenoalkylcarbonylamino having 1 to 5 halogen atoms, C₁-C₈-alkylaminocarbonyloxy, di-(C₁-C₈-alkyl)-aminocarbonyloxy, C₁-C₈-alkyloxycarbonyloxy, C₁-C₈-alkylsulfinyl, C₁-C₈-halogenoalkylsulfinyl having 1 to 5 halogen atoms, C₁-C₈-alkylsulfonyl, C₁-C₈-halogenoalkylsulfonyl having 1 to 5 halogen atoms, C₁-C₈-alkylaminosulfamoyl, di-(C₁-C₈-alkyl)-aminosulfamoyl, C₁-C₆-alkoxyimino-C₁-C₆-alkyl, C₂-C₆-alkenyloxyimino-C₁-C₆-alkyl, C₃-C₆-alkynyloxyimino-C₁-C₆-alkyl, 2-oxopyrrolidin-1-yl, benzyloxyimino-C₁-C₆-alkyl, C₁-C₈-alkoxy-C₁-C₈-alkyl, C₁-C₈-halogenoalkoxy-C₁-C₈-alkyl having 1 to 5 halogen atoms, benzyloxy, benzylsulfanyl, benzylamino, phenoxy, phenylsulfanyl, or phenylamino.

Compounds of the present invention can exist in one or more optical or chiral isomer forms depending on the number of asymmetric centres in the compound. The invention thus relates equally to all the optical isomers and to their racemic or scalemic mixtures (the term “scalemic” denotes a mixture of enantiomers in different proportions) and to the mixtures of all the possible stereoisomers, in all proportions. The diastereoisomers and/or the optical isomers can be separated according to the methods which are known per se by the man ordinary skilled in the art.

Compounds of the present invention can also exist in one or more geometric isomer forms depending on the number of double bonds in the compound. The invention thus relates equally to all geometric isomers and to all possible mixtures, in all proportions. The geometric isomers can be separated according to general methods, which are known per se by the man ordinary skilled in the art.

Compounds of the present invention can also exist in one or more geometric isomer forms depending on the relative position (syn/anti or cis/trans) of the substituents of ring B. The invention thus relates equally to all syn/anti (or cis/trans) isomers and to all possible syn/anti (or cis/trans) mixtures, in all proportions. The syn/anti (or cis/trans) isomers can be separated according to general methods, which are known per se by the man ordinary skilled in the art.

Any of the compounds of formula (I) wherein X represents a hydroxy, a sulfanyl or an amino may be found in its tautomeric form resulting from the shift of the proton of said hydroxy, sulfanyl or amino group. Such tautomeric forms of such compounds are also part of the present invention. More generally speaking, all tautomeric forms of compounds of formula (I) wherein X represents a hydroxy, a sulfanyl group or an amino group, as well as the tautomeric forms of the compounds which can optionally be used as intermediates in the preparation processes and which will be defined in the description of these processes, are also part of the present invention.

A preferred embodiment of the invention is the use of compounds of formula (I) as described above in which T represents sulphur, for controlling nematodes.

Another preferred embodiment of the invention is the use of compounds of formula (IB-1)

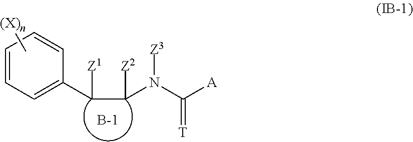

wherein
A represents phenyl of formula A^{a}

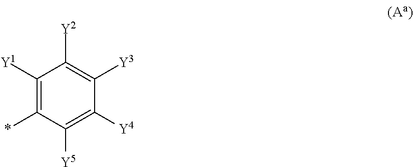

wherein
* indicates the bond which connects A^{a} to the C=T moiety of the compounds of formula (I),
Y¹ represents halogen, nitro, SH, SF₅, CHO, OCHO, NHCHO, cyano, C₁-C₈-alkyl, C₁-C₈-halogenoalkyl having 1 to 5 halogen atoms, C₂-C₈-alkenyl, C₂-C₈-alkynyl, C₃-C₆-cycloalkyl, C₃-C₆-halogenocycloalkyl having 1 to 9 halogen atoms, C₁-C₈-alkylsulfanyl, —C₁-C₈-halogenoalkylsulfanyl having 1 to 5 halogen atoms, C₁-C₈-alkoxy, C₁-C₈-halogenoalkoxy having 1 to 5 halogen atoms, C₁-C₈-alkoxy-C₁-C₈-alkyl, C₂-C₈-alkenyloxy-C₁-C₈-alkyl, C₃-C₈-alkynyloxy-C₁-C₈-alkyl, C₂-C₈-alkenyloxy, C₃-C₈-alkynyloxy, C₁-C₈-alkoxycarbonyl, C₁-C₈-halogenoalkoxycarbonyl having 1 to 5 halogen atoms, C₁-C₈-alkylcarbonyloxy, C₁-C₈-halogenoalkylcarbonyloxy having 1 to 5 halogen atoms, C₁-C₈-alkylsulfinyl, C₁-C₈-halogenoalkylsulfinyl having 1 to 5 halogen atoms, C₁-C₈-alkylsulfonyl, C₁-C₈-halogenoalkylsulfonyl having 1 to 5 halogen atoms, C₁-C₈-alkylsulfonamide, substituted or unsubstituted tri-(C₁-C₈)-alkylsilyl, substituted or unsubstituted aryl and substituted or unsubstituted aryloxy;
Y², Y³, Y⁴ and Y⁵ independently from each other represent hydrogen or Y¹; or
A represents a carbo-linked heterocyclyl group optionally substituted by 1 to five groups R, wherein
R independently from each other R represents hydrogen, halogen, nitro, cyano, hydroxy, amino, sulfanyl, pentafluoro-λ⁶-sulfanyl, substituted or unsubstituted (C₁-C₈-alkoxyimino)-C₁-C₈-alkyl, substituted or unsubstituted (benzyloxyimino)-C₁-C₈-alkyl, substituted or unsubstituted C₁-C₈-alkyl, C₁-C₈-halogenoalkyl having 1 to 5 halogen atoms, substituted or unsubstituted C₂-C₈-alkenyl, C₂-C₈-halogenoalkenyl having 1 to 5 halogen atoms, substituted or unsubstituted C₂-C₈-alkynyl, C₂-C₈-halogenoalkynyl having 1 to 5 halogen atoms, substituted or unsubstituted C₁-C₈-alkoxy, C₁-C₈-halogenoalkoxy having 1 to 5 halogen atoms, substituted or unsubstituted C₁-C₈-alkylsulfanyl, C₁-C₈-halogenoalkylsulfanyl having 1 to 5 halogen atoms, substituted or unsubstituted C₁-C₈-alkylsulfinyl, C₁-C₈-halogenoalkylsulfinyl having 1 to 5 halogen atoms, substituted or unsubstituted C₁-C₈-alkylsulfonyl, C₁-C₈-halogenoalkylsulfonyl having 1 to 5 halogen atoms, substituted or unsubstituted C₁-C₈-alkylamino, substituted or unsubstituted di-C₁-C₈-alkylamino, substituted or unsubstituted C₂-C₈-alkenyloxy, substituted or unsubstituted C₃-C₈-alkynyloxy, substituted or unsubstituted C₃-C₇-cycloalkyl, C₃-C₇-halogenocycloalkyl having 1 to 5 halogen atoms, substituted or unsubstituted tri-(C₁-C₈-alkyl)-silyl, substituted or unsubstituted C₁-C₈-alkylcarbonyl, C₁-C₈-halogenoalkylcarbonyl having 1 to 5 halogen atoms, substituted or unsubstituted C₁-C₈-alkoxycarbonyl, C₁-C₈-halogenoalkoxycarbonyl having 1 to 5 halogen atoms, substituted or unsubstituted C₁-C₈-alkylcarbamoyl, substituted or unsubstituted di-(C₁-C₈)-alkylcarbamoyl, phenoxy, phenylsulfanyl, phenylamino, benzyloxy, benzylsulfanyl, or benzylamino,
T represents oxygen or sulphur,
B-1 represents a 5-, 6- or 7-membered non-aromatic carbocycle optionally substituted by substituents selected from the group consisting of one to four C₁-C₈-alkyl groups and one to four halogen atoms,
n represents 0, 1, 2, 3, 4 or 5,
X independently from each other X represents halogen, nitro, cyano, isonitrile, hydroxy, amino, sulfanyl, pentafluoro-λ⁶-sulfanyl, formyl, formyloxy, formylamino, substituted or unsubstituted (hydroxyimino)-C₁-C₈-alkyl, substituted or unsubstituted (C₁-C₈-alkoxyimino)-C₁-C₈-alkyl, substituted or unsubstituted (C₂-C₈-alkenyloxyimino)-C₁-C₈-alkyl, substituted or unsubstituted (C₃-C₈-alkynyloxyimino)-C₁-C₈-alkyl, substituted or unsubstituted (benzyloxyimino)-C₁-C₈-alkyl, carboxy, carbamoyl, thiocarbamoyl, N-hydroxycarbamoyl, carbamate, substituted or unsubstituted C₁-C₈-alkyl, C₁-C₈-halogenoalkyl having 1 to 9 halogen atoms, substituted or unsubstituted C₂-C₈-alkenyl, C₂-C₈-halogenoalkenyl having 1 to 9 halogen atoms, substituted or unsubstituted C₂-C₈-alkynyl, C₂-C₈-halogenoalkynyl having 1 to 9 halogen atoms, substituted or unsubstituted C₁-C₈-alkoxy, C₁-C₈-halogenoalkoxy having 1 to 9 halogen atoms, substituted or unsubstituted C₁-C₈-alkylsulfanyl, C₁-C₈-halogenoalkylsulfanyl having 1 to 9 halogen atoms, substituted or unsubstituted C₁-C₈-alkylsulfinyl, C₁-C₈-halogenoalkylsulfinyl having 1 to 9 halogen atoms, substituted or unsubstituted C₁-C₈-alkylsulfonyl, C₁-C₈-halogenoalkylsulfonyl having 1 to 9 halogen atoms, substituted or unsubstituted C₁-C₈-alkylamino, substituted or unsubstituted di-(C₁-C₈-alkyl)-amino, substituted or unsubstituted C₂-C₈-alkenyloxy, C₂-C₈-halogenoalkenyloxy having 1 to 9 halogen atoms, substituted or unsubstituted C₃-C₈-alkynyloxy, C₃-C₈-halogenoalkynyloxy having 1 to 9 halogen atoms, substituted or unsubstituted C₃-C₇-cycloalkyl, C₃-C₇-halogenocycloalkyl having 1 to 9 halogen atoms, substituted or unsubstituted C₃-C₇-cycloalkyloxy, substituted or unsubstituted C₄-C₇-cycloalkenyl, C₄-C₇-halogenocycloalkenyl having 1 to 9 halogen atoms, substituted or unsubstituted (C₃-C₇-cycloalkyl)-C₁-C₈-alkyl, substituted or unsubstituted (C₃-C₇-cycloalkyl)-C₂-C₈-alkenyl, substituted or unsubstituted (C₃-C₇-cycloalkyl)-C₂-C₈-alkynyl, substituted or unsubstituted tri-(C₁-C₈-alkyl)-silyl, substituted or unsubstituted tri-(C₁-C₈-alkyl)-silyl-C₁-C₈-alkyl, substituted or unsubstituted C₁-C₈-alkylcarbonyl, C₁-C₈-halogenoalkylcarbonyl having 1 to 9 halogen atoms, substituted or unsubstituted C₁-C₈-alkylcarbonyloxy, C₁-C₈-halogenoalkylcarbonyloxy having 1 to 9 halogen atoms, substituted or unsubstituted C₁-C₈-alkylcarbonylamino, C₁-C₈-halogenoalkylcarbonylamino having 1 to 9 halogen atoms, substituted or unsubstituted C₁-C₈-alkoxycarbonyl, C₁-C₈-halogenoalkoxycarbonyl having 1 to 9 halogen atoms, substituted or unsubstituted C₁-C₈-alkyloxycarbonyloxy, C₁-C₈-halogenoalkoxycarbonyloxy having 1 to 9 halogen atoms, substituted or unsubstituted C₁-C₈-alkylcarbamoyl, substituted or unsubstituted di-C₁-C₈-alkylcarbamoyl, substituted or unsubstituted C₁-C₈-alkylaminocarbonyloxy, substituted or unsubstituted di-C₁-C₈-alkylaminocarbonyloxy, substituted or unsubstituted N—(C₁-C₈-alkyl)-hydroxycarbamoyl, substituted or unsubstituted C₁-C₈-alkoxycarbamoyl, substituted or unsubstituted N—(C₁-C₈-alkyl)-C₁-C₈-alkoxycarbamoyl, aryl optionally substituted by 1 to 6 groups Q which can be the same or different, aryl-C₁-C₈-alkyl optionally substituted by 1 to 6 groups Q which can be the same or different, aryl-C₂-C₈-alkenyl optionally substituted by 1 to 6 groups Q which can be the same or different, aryl-C₂-C₈-alkynyl optionally substituted by 1 to 6 groups Q which can be the same or different, aryloxy optionally substituted by 1 to 6 groups Q which can be the same or different, arylsulfanyl optionally substituted by 1 to 6 groups Q which can be the same or different, arylamino optionally substituted by 1 to 6 groups Q which can be the same or different, aryl-C₁-C₈-alkyloxy optionally substituted by 1 to 6 groups Q which can be the same or different, aryl-C₁-C₈-alkylsulfanyl optionally substituted by 1 to 6 groups Q which can be the same or different, aryl-C₁-C₈-alkylamino optionally substituted by 1 to 6 groups Q which can be the same or different, pyridinyl which can be substituted by 1 to 4 groups Q, pyridinyloxy which is optionally substituted by 1 to 4 groups Q, or
two substituents X together with the carbon atoms to which they are attached form a 5- or 6-membered, saturated carbocycle or saturated heterocycle, which is optionally substituted by 1 to 4 groups Q which can be the same or different,
Z¹ and Z² independently represent hydrogen, halogen, cyano, substituted or unsubstituted C₁-C₈-alkyl, C₁-C₈-halogenoalkyl having 1 to 5 halogen atoms, substituted or unsubstituted C₁-C₈-alkoxy, substituted or unsubstituted C₁-C₈-alkylsulfanyl, or substituted or unsubstituted C₁-C₈-alkoxycarbonyl,
Z³ represents hydrogen, cyano, C₁-C₈-alkoxy, unsubstituted C₃-C₇-cycloalkyl or C₃-C₇-cycloalkyl substituted by 1 to 10 substituents that can be the same or different, selected from the list consisting of halogen, cyano, C₁-C₈-alkyl, C₁-C₈-halogenoalkyl comprising 1 to 9 halogen atoms, C₁-C₈-alkoxy, C₁-C₈-halogenoalkoxy comprising 1 to 9 halogen atoms, C₁-C₈-alkoxycarbonyl, C₁-C₈-halogenoalkoxycarbonyl comprising 1 to 9 halogen atoms, C₁-C₈-alkylaminocarbonyl and di-(C₁-C₈-alkyl)-amino,
Q represents halogen, cyano, nitro, substituted or unsubstituted C₁-C₈-alkyl, C₁-C₈-halogenoalkyl having 1 to 9 halogen atoms, substituted or unsubstituted C₁-C₈-alkoxy, C₁-C₈-halogenoalkoxy having 1 to 9 halogen atoms, substituted or unsubstituted C₁-C₈-alkylsulfanyl, C₁-C₈-halogenoalkylsulfanyl having 1 to 9 halogen atoms, substituted or unsubstituted tri-(C₁-C₈-alkyl)-silyl, substituted or unsubstituted tri-(C₁-C₈-alkyl)-silyl-C₁-C₈-alkyl, substituted or unsubstituted C₁-C₈-alkoxyimino-C₁-C₈-alkyl, substituted or unsubstituted (benzyloxyimino)-C₁-C₈-alkyl,
and N-oxides, metallic complexes, metalloidic complexes and optically active isomers of the compounds of formula (I),
for controlling nematodes.

Another preferred embodiment of the invention is the use of compounds of formula (I) or formula (IB-1) wherein A is a group of formula A^{a}.

Another preferred embodiment of the invention is the use of compounds of formula (I) or formula (IB-1) wherein A is a heterocycle of formula (A¹)

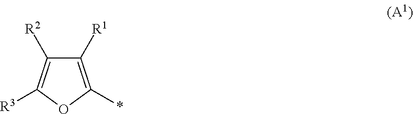

wherein
R¹ to R³ independently from each other represent hydrogen, halogen, substituted or unsubstituted C₁-C₅-alkyl, C₁-C₅-halogenoalkyl comprising 1 to 9 halogen atoms, substituted or unsubstituted C₁-C₅-alkoxy or C₁-C₅-halogenoalkoxy comprising 1 to 9 halogen atoms.

Another preferred embodiment of the invention is the use of compounds of formula (I) or formula (IB-1) wherein A is a heterocycle of formula (A²)

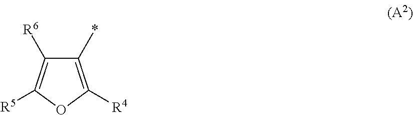

wherein
R⁴ to R⁶ independently from each other represent hydrogen, halogen, substituted or unsubstituted C₁-C₅-alkyl, C₁-C₅-halogenoalkyl comprising 1 to 9 halogen atoms, substituted or unsubstituted C₁-C₅-alkoxy or C₁-C₅-halogenoalkoxy comprising 1 to 9 halogen atoms.

Another preferred embodiment of the invention is the use of compounds of formula (I) or formula (IB-1) wherein A is a heterocycle of formula (A³)

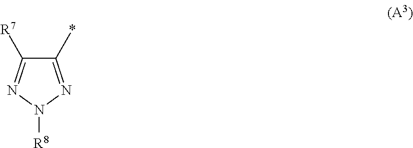

wherein
R⁷ represents hydrogen, halogen, substituted or unsubstituted C₁-C₅-alkyl, C₁-C₅-halogenoalkyl comprising 1 to 9 halogen atoms, substituted or unsubstituted C₁-C₅-alkoxy or C₁-C₅-halogenoalkoxy comprising 1 to 9 halogen atoms, and
R⁸ represents hydrogen or substituted or unsubstituted C₁-C₅-alkyl.

Another preferred embodiment of the invention is the use of compounds of formula (I) or formula (IB-1) wherein A is a heterocycle of formula (A⁴)

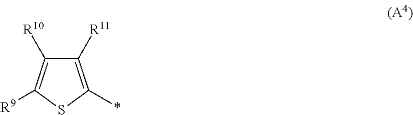

wherein
R⁹ to R¹¹ independently from each other represent hydrogen, halogen, substituted or unsubstituted C₁-C₅-alkyl, amino, substituted or unsubstituted C₁-C₅-alkoxy, substituted or unsubstituted C₁-C₅-alkylsulphanyl, C₁-C₅-halogenoalkyl comprising 1 to 9 halogen atoms or C₁-C₅-halogenoalkoxy comprising 1 to 9 halogen atoms.

Another preferred embodiment of the invention is the use of compounds of formula (I) or formula (IB-1) wherein A is a heterocycle of formula (A⁵)

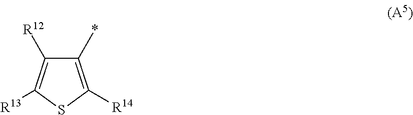

wherein
R¹² and R¹³ independently from each other represent hydrogen, halogen, substituted or unsubstituted C₁-C₅-alkyl, substituted or unsubstituted C₁-C₅-alkoxy, amino, C₁-C₅-halogenoalkyl comprising 1 to 9 halogen atoms or C₁-C₅-halogenoalkoxy comprising 1 to 9 halogen atoms, and
R¹⁴ represents hydrogen, halogen, substituted or unsubstituted C₁-C₅-alkyl, substituted or unsubstituted C₁-C₅-alkoxy, amino, C₁-C₅-halogenoalkyl comprising 1 to 9 halogen atoms or C₁-C₅-halogenoalkoxy comprising 1 to 9 halogen atoms.

Another preferred embodiment of the invention is the use of compounds of formula (I) or formula (IB-1) wherein A is a heterocycle of formula (A⁶)

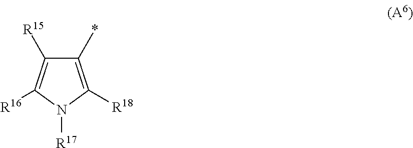

wherein
R¹⁵ represents hydrogen, halogen, cyano, substituted or unsubstituted C₁-C₅-alkyl, substituted or unsubstituted C₁-C₅-alkoxy, C₁-C₅-halogenoalkoxy comprising 1 to 9 halogen atoms or C₁-C₅-halogenoalkyl comprising 1 to 9 halogen atoms, and
R¹⁶ and R¹⁸ independently from each other represent hydrogen, halogen, substituted or unsubstituted C₁-C₅-alkoxycarbonyl, substituted or unsubstituted C₁-C₅-alkyl, C₁-C₅-halogenoalkoxy comprising 1 to 9 halogen atoms or C₁-C₅-halogenoalkyl comprising 1 to 9 halogen atoms, and
R¹⁷ represents hydrogen or substituted or unsubstituted C₁-C₅-alkyl.

Another preferred embodiment of the invention is the use of compounds of formula (IB-1) wherein A is a heterocycle of formula (A⁷)

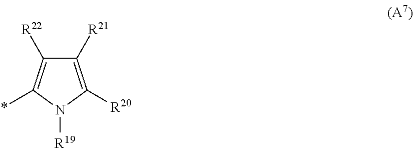

wherein
R¹⁹ represents hydrogen or substituted or unsubstituted C₁-C₅-alkyl, and
R²⁰ to R²² independently from each other represent hydrogen, halogen, substituted or unsubstituted C₁-C₅-alkyl or C₁-C₅-halogenoalkyl comprising 1 to 9 halogen atoms.

Another preferred embodiment of the invention is the use of compounds of formula (I) or formula (IB-1)
wherein A is a heterocycle of formula (A⁸)

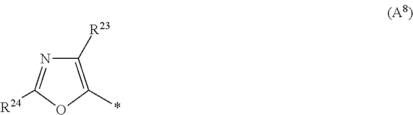

wherein
R²³ represents hydrogen, halogen, substituted or unsubstituted C₁-C₅-alkyl or C₁-C₅-halogenoalkyl comprising 1 to 9 halogen atoms, and
R²⁴ represents hydrogen or substituted or unsubstituted C₁-C₅-alkyl or C₁-C₅-halogenoalkyl comprising 1 to 9 halogen atoms.

Another preferred embodiment of the invention is the use of compounds of formula (I) or formula (IB-1) wherein A is a heterocycle of formula (A⁹)

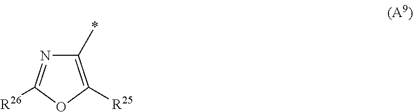

wherein
R²⁵ represents hydrogen, halogen, substituted or unsubstituted C₁-C₅-alkyl or C₁-C₅-halogenoalkyl comprising 1 to 9 halogen atoms, and
R²⁶ represents hydrogen, substituted or unsubstituted C₁-C₅-alkyl or C₁-C₅-halogenoalkyl comprising 1 to 9 halogen atoms.

Another preferred embodiment of the invention is the use of compounds of formula (I) or formula (IB-1) wherein A is a heterocycle of formula (A¹⁰)

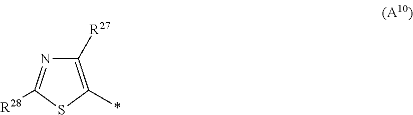

wherein
R²⁷ represents hydrogen, halogen, substituted or unsubstituted C₁-C₅-alkyl or C₁-C₅-halogenoalkyl comprising 1 to 9 halogen atoms, and
R²⁸ represents hydrogen, halogen, substituted or unsubstituted C₁-C₅-alkyl, C₁-C₅-halogenoalkyl comprising 1 to 9 halogen atoms, C₁-C₅-halogenoalkoxy comprising 1 to 9 halogen atoms, amino, substituted or unsubstituted C₁-C₅-alkylamino or substituted or unsubstituted di-(C₁-C₅-alkyl)-amino.

Another preferred embodiment of the invention is the use of compounds of formula (I) or formula (IB-1) wherein A is a heterocycle of formula (A¹¹)

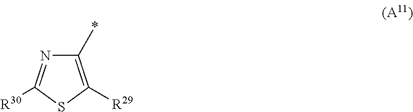

wherein
R²⁹ represents hydrogen, halogen, substituted or unsubstituted C₁-C₅-alkyl, substituted or unsubstituted C₁-C₅-alkoxy, C₁-C₅-halogenoalkoxy comprising 1 to 9 halogen atoms or C₁-C₅-halogenoalkyl comprising 1 to 9 halogen atoms, and
R³⁰ represents hydrogen, halogen, substituted or unsubstituted C₁-C₅-alkyl, C₁-C₅-halogenoalkyl comprising 1 to 9 halogen atoms, C₁-C₅-halogenoalkoxy comprising 1 to 9 halogen atoms, amino, substituted or unsubstituted C₁-C₅-alkylamino or substituted or unsubstituted di-(C₁-C₅-alkyl)-amino.

Another preferred embodiment of the invention is the use of compounds of formula (I) or formula (IB-1) wherein A is a heterocycle of formula (A¹²)

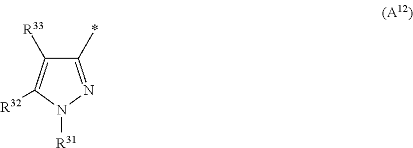

wherein
R³¹ represents hydrogen or substituted or unsubstituted C₁-C₅-alkyl, and
R³² represents hydrogen, halogen, substituted or unsubstituted C₁-C₅-alkyl or C₁-C₅-halogenoalkyl comprising 1 to 9 halogen atoms, and
R³³ represents hydrogen, halogen, nitro, substituted or unsubstituted C₁-C₅-alkyl, substituted or unsubstituted C₁-C₅-alkoxy, C₁-C₅-halogenoalkoxy comprising 1 to 9 halogen atoms or C₁-C₅-halogenoalkyl comprising 1 to 9 halogen atoms.

Another preferred embodiment of the invention is the use of compounds of formula (I) or formula (IB-1) wherein A is a heterocycle of formula (A¹³)

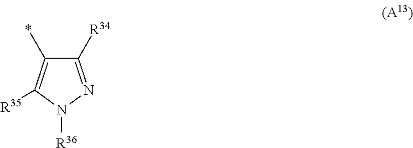

wherein
R³⁴ represents hydrogen, halogen, substituted or unsubstituted C₁-C₅-alkyl, substituted or unsubstituted C₃-C₅-cycloalkyl, C₁-C₅-halogenoalkyl comprising 1 to 9 halogen atoms, substituted or unsubstituted C₁-C₅-alkoxy, substituted or unsubstituted C₃-C₅-alkynyloxy or C₁-C₅-halogenoalkoxy comprising 1 to 9 halogen atoms, and
R³⁵ represents hydrogen, halogen, substituted or unsubstituted C₁-C₅-alkyl, cyano, substituted or unsubstituted C₁-C₅-alkoxy, substituted or unsubstituted C₁-C₅-alkylsulphanyl, C₁-C₅-halogenoalkyl comprising 1 to 9 halogen atoms, C₁-C₅-halogenoalkoxy comprising 1 to 9 halogen atoms, amino, substituted or unsubstituted C₁-C₅-alkylamino or substituted or unsubstituted di-(C₁-C₅-alkyl)-amino, and
R³⁶ represents a hydrogen atom or substituted or unsubstituted C₁-C₅-alkyl.

Another preferred embodiment of the invention is the use of compounds of formula (I) or formula (IB-1) wherein A is a heterocycle of formula (A¹⁴)

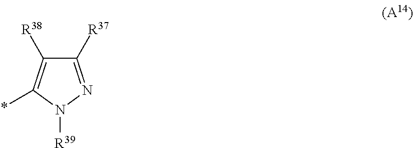

wherein
R³⁷ and R³⁸ independently from each other represent hydrogen, halogen, substituted or unsubstituted C₁-C₅-alkyl, C₁-C₅-halogenoalkyl comprising 1 to 9 halogen atoms, substituted or unsubstituted C₁-C₅-alkoxy or a substituted or unsubstituted C₁-C₅-alkylsulphanyl, and
R³⁹ represents hydrogen or substituted or unsubstituted C₁-C₅-alkyl.

Another preferred embodiment of the invention is the use of compounds of formula (I) or formula (IB-1) wherein A is a heterocycle of formula (A¹⁵)

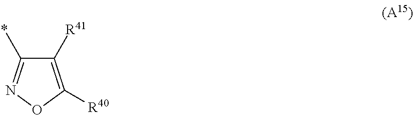

wherein
R⁴⁰ and R⁴¹ independently from each other represent hydrogen, halogen, substituted or unsubstituted C₁-C₅-alkyl or C₁-C₅-halogenoalkyl comprising 1 to 9 halogen atoms.

Another preferred embodiment of the invention is the use of compounds of formula (I) or formula (IB-1) wherein A is a heterocycle of formula (A¹⁶)

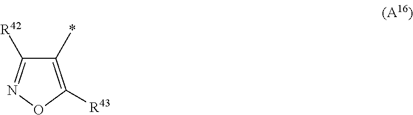

wherein
R⁴² and R⁴³ independently from each other represent hydrogen, halogen, substituted or unsubstituted C₁-C₅-alkyl, C₁-C₅-halogenoalkyl comprising 1 to 9 halogen atoms or amino.

Another preferred embodiment of the invention is the use of compounds of formula (I) or formula (IB-1) wherein A is a heterocycle of formula (A¹⁷)

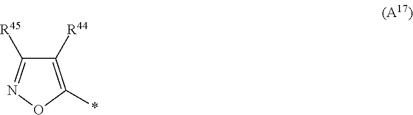

wherein
R⁴⁴ and R⁴⁵ independently from each other represent hydrogen, halogen, substituted or unsubstituted C₁-C₅-alkyl or C₁-C₅-halogenoalkyl comprising 1 to 9 halogen atoms.

Another preferred embodiment of the invention is the use of compounds of formula (I) or formula (IB-1) wherein A is a heterocycle of formula (A¹⁸)

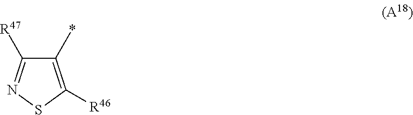

wherein
R⁴⁷ represents hydrogen, halogen, substituted or unsubstituted C₁-C₅-alkyl or C₁-C₅-halogenoalkyl comprising 1 to 9 halogen atoms, and
R⁴⁶ represents hydrogen, halogen, substituted or unsubstituted C₁-C₅-alkyl, C₁-C₅-halogenoalkyl comprising 1 to 9 halogen atoms or substituted or unsubstituted C₁-C₅-alkylsulfanyl.

Another preferred embodiment of the invention is the use of compounds of formula (I) or formula (IB-1) wherein A is a heterocycle of formula (A¹⁹)

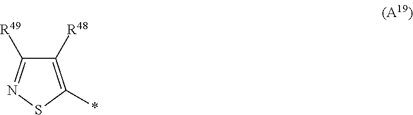

wherein
R⁴⁹ and R⁴⁸ independently from each other represent hydrogen, halogen, substituted or unsubstituted C₁-C₅-alkyl, substituted or unsubstituted C₁-C₅-alkoxy, C₁-C₅-halogenoalkoxy comprising 1 to 9 halogen atoms or C₁-C₅-halogenoalkyl comprising 1 to 9 halogen atoms.

Another preferred embodiment of the invention is the use of compounds of formula (I) or formula (IB-1) wherein A is a heterocycle of formula (A²⁰)

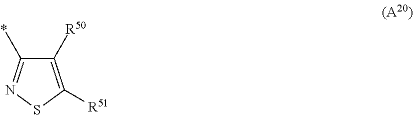

wherein
R⁵⁰ and R⁵¹ independently from each other represent hydrogen, halogen, substituted or unsubstituted C₁-C₅-alkyl, substituted or unsubstituted C₁-C₅-alkoxy, C₁-C₅-halogenoalkoxy comprising 1 to 9 halogen atoms or C₁-C₅-halogenoalkyl comprising 1 to 9 halogen atoms.

Another preferred embodiment of the invention is the use of compounds of formula (I) or formula (IB-1) wherein A is a heterocycle of formula (A²¹)

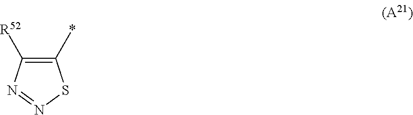

wherein
R⁵² represents hydrogen, halogen, substituted or unsubstituted C₁-C₅-alkyl or C₁-C₅-halogenoalkyl comprising 1 to 9 halogen atoms.

Another preferred embodiment of the invention is the use of compounds of formula (I) or formula (IB-1) wherein A is a heterocycle of formula (A²²)

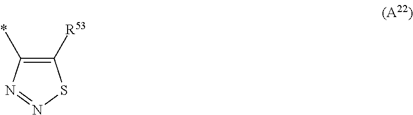

wherein
R⁵³ represents hydrogen, halogen, substituted or unsubstituted C₁-C₅-alkyl or C₁-C₅-halogenoalkyl comprising 1 to 9 halogen atoms.

Another preferred embodiment of the invention is the use of compounds of formula (I) or formula (IB-1) wherein A is a heterocycle of formula (A²³)

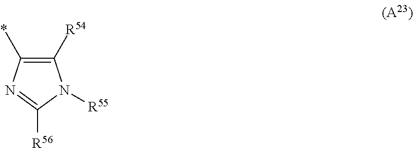

wherein
R⁵⁴ and R⁵⁶ independently from each other represent hydrogen, halogen, substituted or unsubstituted C₁-C₅-alkyl or C₁-C₅-halogenoalkyl comprising 1 to 9 halogen atoms, and
R⁵⁵ represents hydrogen or substituted or unsubstituted C₁-C₅-alkyl.

Another preferred embodiment of the invention is the use of compounds of formula (I) or formula (IB-1) wherein A is a heterocycle of formula (A²⁴)

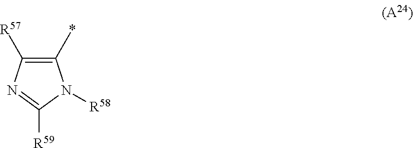

wherein
R⁵⁷ and R⁵⁹ independently from each other represent hydrogen, halogen, substituted or unsubstituted C₁-C₅-alkyl or C₁-C₅-halogenoalkyl comprising 1 to 9 halogen atoms, and
R⁵⁸ represents hydrogen or substituted or unsubstituted C₁-C₅-alkyl.

Another preferred embodiment of the invention is the use of compounds of formula (I) or formula (IB-1) wherein A is a heterocycle of formula (A²⁵)

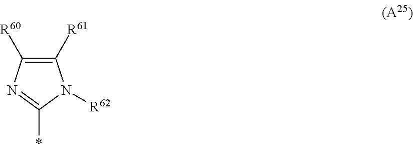

wherein
R⁶⁰ and R⁶¹ independently from each other represent hydrogen, halogen, substituted or unsubstituted C₁-C₅-alkyl or C₁-C₅-halogenoalkyl comprising 1 to 9 halogen atoms, and
R⁶² represents a hydrogen atom or substituted or unsubstituted C₁-C₅-alkyl.

Another preferred embodiment of the invention is the use of compounds of formula (I) or formula (IB-1) wherein A is a heterocycle of formula (A²⁶)

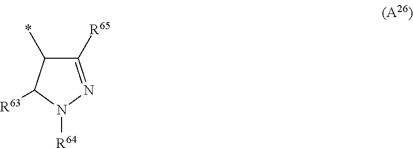

wherein
R⁶³ represents hydrogen, halogen, substituted or unsubstituted C₁-C₅-alkyl, cyano, substituted or unsubstituted C₁-C₅-alkoxy, substituted or unsubstituted C₁-C₅-alkylsulphanyl, C₁-C₅-halogenoalkyl comprising 1 to 9 halogen atoms, C₁-C₅-halogenoalkoxy comprising 1 to 9 halogen atoms, amino, substituted or unsubstituted C₁-C₅-alkylamino or di(C₁-C₅-alkyl)amino, and
R⁶⁴ represents hydrogen or substituted or unsubstituted C₁-C₅-alkyl, and
R⁶⁵ represents hydrogen, halogen, substituted or unsubstituted C₁-C₅-alkyl, substituted or unsubstituted C₃-C₅-cycloalkyl, C₁-C₅-halogenoalkyl comprising 1 to 9 halogen atoms, substituted or unsubstituted C₁-C₅-alkoxy, substituted or unsubstituted C₃-C₅-alkynyloxy or C₁-C₅-halogenoalkoxy comprising 1 to 9 halogen atoms.

Another preferred embodiment of the invention is the use of compounds of formula (I) or formula (IB-1) wherein A is a heterocycle of formula (A²⁷)

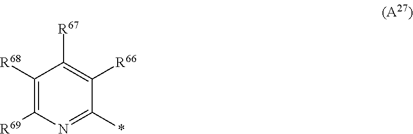

in which
R⁶⁶ represents hydrogen, halogen, hydroxy, cyano, C₁-C₄-alkyl, C₁-C₄-halogenoalkyl having 1 to 5 halogen atoms, C₁-C₄-alkoxy, C₁-C₄-alkylsulfanyl, C₁-C₄-halogenoalkylsulfanyl having 1 to 5 halogen atoms and C₁-C₄-halogenoalkoxy having 1 to 5 halogen atoms, and
R⁶⁷, R⁶⁸ and R⁶⁹ independently from each other represent hydrogen, halogen, cyano, C₁-C₄-alkyl, C₁-C₄-halogenoalkyl having 1 to 5 halogen atoms, C₁-C₄-alkoxy, C₁-C₄-alkylsulfanyl, C₁-C₄-halogenoalkoxy having 1 to 5 halogen atoms, SC₁-C₄-alkylsulfinyl and C₁-C₄-alkylsulfonyl.

Another preferred embodiment of the invention is the use of compounds of formula (I) or formula (IB-1) wherein A is a heterocycle of formula (A²⁸)

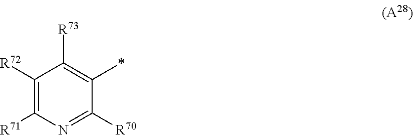

in which
R⁷⁰ represents hydrogen, halogen, hydroxy, cyano, C₁-C₄-alkyl, C₁-C₄-halogenoalkyl having 1 to 5 halogen atoms, C₁-C₄-alkoxy, C₁-C₅-alkylsulfanyl, C₂-C₅-alkenylsulfanyl, C₁-C₄-halogenoalkylsulfanyl having 1 to 5 halogen atoms, C₁-C₄-halogenoalkoxy having 1 to 5 halogen atoms, phenyloxy (optionally substituted by halogen or C₁-C₄-alkyl) and phenylsulfanyl (optionally substituted by halogen or C₁-C₄-alkyl), and
R⁷¹, R⁷² and R⁷³ independently from each other represent hydrogen, halogen, cyano, C₁-C₄-alkyl, C₁-C₄-halogenoalkyl having 1 to 5 halogen atoms, C₁-C₄-alkoxy, C₁-C₄-alkylsulfanyl, C₁-C₄-halogenoalkoxy having 1 to 5 halogen atoms, C₁-C₄-alkylsulfinyl, C₁-C₄-alkylsulfonyl, N-morpholine optionally substituted by halogen or C₁-C₄-alkyl, and thienyl (optionally substituted by halogen or C₁-C₄-alkyl).

Another preferred embodiment of the invention is the use of compounds of formula (I) or formula (IB-1) wherein A is a heterocycle of formula (A²⁹)

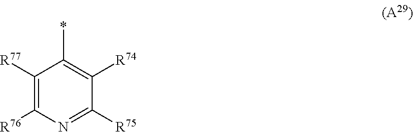

in which
R⁷⁴, R⁷⁵, R⁷⁶ and R⁷⁷ independently from each other represent hydrogen, halogen, hydroxy, cyano, C₁-C₄-alkyl, C₁-C₄-halogenoalkyl having 1 to 5 halogen atoms, C₁-C₄-alkoxy, C₁-C₄-alkylsulfanyl, C₁-C₄-halogenoalkylsulfanyl having 1 to 5 halogen atoms, C₁-C₄-halogenoalkoxy having 1 to 5 halogen atoms, C₁-C₄-alkylsulfinyl and C₁-C₄-alkylsulfonyl.

Another preferred embodiment of the invention is the use of compounds of formula (I) or formula (IB-1)
wherein A is a heterocycle of formula (A³⁰)

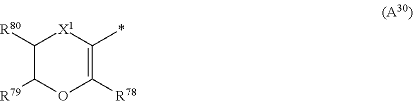

in which
X¹ represents —S—, —SO—, —SO₂— and —CH₂—, and
R⁷⁸ represents C₁-C₄-alkyl and C₁-C₄-halogenoalkyl having 1 to 5 halogen atoms, and
R⁷⁹ and R⁸⁰ independently from each other represent hydrogen and C₁-C₄-alkyl.

Another preferred embodiment of the invention is the use of compounds of formula (I) or formula (IB-1) wherein A is a heterocycle of formula (A³¹)

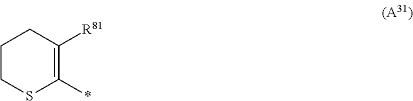

in which
R⁸¹ represents C₁-C₄-alkyl and C₁-C₄-halogenoalkyl having 1 to 5 halogen atoms.

Another preferred embodiment of the invention is the use of compounds of formula (I) or formula (IB-1) wherein A is a heterocycle of formula (A³²)

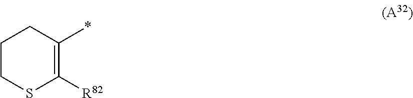

in which
R⁸² represents C₁-C₄-alkyl and C₁-C₄-halogenoalkyl having 1 to 5 halogen atoms.

Another preferred embodiment of the invention is the use of compounds of formula (I) or formula (IB-1) wherein A is a heterocycle of formula (A³³)

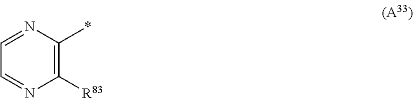

in which
R⁸³ represents hydrogen, halogen, C₁-C₄-alkyl and C₁-C₄-halogenoalkyl having 1 to 5 halogen atoms.

The * in formulae A¹ to A³³ indicates the bond which connects A¹ to A³³ to the C=T moiety of the compounds of formula (I) or formula (IB-1).

Another preferred embodiment of the invention is the use of compounds of formula (IB-2)

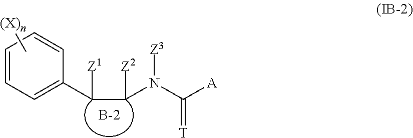

wherein
A represents a carbo-linked heterocyclyl group selected from the group consisting of A²⁶, A³⁰, A³¹, and A³²,
Y¹ represents halogen, nitro, SH, SF₅, CHO, OCHO, NHCHO, cyano, C₁-C₈-alkyl, C₁-C₈-halogenoalkyl having 1 to 5 halogen atoms, C₂-C₈-alkenyl, C₂-C₈-alkynyl, C₃-C₆-cycloalkyl, C₃-C₆-halogenocycloalkyl having 1 to 9 halogen atoms, C₁-C₈-alkylsulfanyl, —C₁-C₈-halogenoalkylsulfanyl having 1 to 5 halogen atoms, C₁-C₈-alkoxy, C₁-C₈-halogenoalkoxy having 1 to 5 halogen atoms, C₁-C₈-alkoxy-C₁-C₈-alkyl, C₂-C₈-alkenyloxy-C₁-C₈-alkyl, C₃-C₈-alkynyloxy-C₁-C₈-alkyl, C₂-C₈-alkenyloxy, C₃-C₈-alkynyloxy, C₁-C₈-alkoxycarbonyl, C₁-C₈-halogenoalkoxycarbonyl having 1 to 5 halogen atoms, C₁-C₈-alkylcarbonyloxy, C₁-C₈-halogenoalkylcarbonyloxy having 1 to 5 halogen atoms, C₁-C₈-alkylsulfinyl, C₁-C₈-halogenoalkylsulfinyl having 1 to 5 halogen atoms, C₁-C₈-alkylsulfonyl, C₁-C₈-halogenoalkylsulfonyl having 1 to 5 halogen atoms, C₁-C₈-alkylsulfonamide, substituted or unsubstituted tri-(C₁-C₈)-alkylsilyl, substituted or unsubstituted aryl and substituted or unsubstituted aryloxy;
Y², Y³, Y⁴ and Y⁵ independently from each other represent hydrogen or Y¹,
R independently from each other R represents hydrogen, halogen, nitro, cyano, hydroxy, amino, sulfanyl, pentafluoro-λ⁶-sulfanyl, substituted or unsubstituted (C₁-C₈-alkoxyimino)-C₁-C₈-alkyl, substituted or unsubstituted (benzyloxyimino)-C₁-C₈-alkyl, substituted or unsubstituted C₁-C₈-alkyl, C₁-C₈-halogenoalkyl having 1 to 5 halogen atoms, substituted or unsubstituted C₂-C₈-alkenyl, C₂-C₈-halogenoalkenyl having 1 to 5 halogen atoms, substituted or unsubstituted C₂-C₈-alkynyl, C₂-C₈-halogenoalkynyl having 1 to 5 halogen atoms, substituted or unsubstituted C₁-C₈-alkoxy, C₁-C₈-halogenoalkoxy having 1 to 5 halogen atoms, substituted or unsubstituted C₁-C₈-alkylsulfanyl, C₁-C₈-halogenoalkylsulfanyl having 1 to 5 halogen atoms, substituted or unsubstituted C₁-C₈-alkylsulfinyl, C₁-C₈-halogenoalkylsulfinyl having 1 to 5 halogen atoms, substituted or unsubstituted C₁-C₈-alkylsulfonyl, C₁-C₈-halogenoalkylsulfonyl having 1 to 5 halogen atoms, substituted or unsubstituted C₁-C₈-alkylamino, substituted or unsubstituted di-C₁-C₈-alkylamino, substituted or unsubstituted C₂-C₈-alkenyloxy, substituted or unsubstituted C₃-C₈-alkynyloxy, substituted or unsubstituted C₃-C₇-cycloalkyl, C₃-C₇-halogenocycloalkyl having 1 to 5 halogen atoms, substituted or unsubstituted tri-(C₁-C₈-alkyl)-silyl, substituted or unsubstituted C₁-C₈-alkylcarbonyl, C₁-C₈-halogenoalkylcarbonyl having 1 to 5 halogen atoms, substituted or unsubstituted C₁-C₈-alkoxycarbonyl, C₁-C₈-halogenoalkoxycarbonyl having 1 to 5 halogen atoms, substituted or unsubstituted C₁-C₈-alkylcarbamoyl, substituted or unsubstituted di-(C₁-C₈)-alkylcarbamoyl, phenoxy, phenylsulfanyl, phenylamino, benzyloxy, benzylsulfanyl, or benzylamino,
T represents oxygen or sulphur,
B-2 represents a 3- or 4-membered non-aromatic carbocycle optionally substituted by substituents selected from the group consisting of one to four C₁-C₈-alkyl groups and one to four halogen atoms,
n represents 0, 1, 2, 3, 4 or 5,
X independently from each other X represents halogen, nitro, cyano, isonitrile, hydroxy, amino, sulfanyl, pentafluoro-λ⁶-sulfanyl, formyl, formyloxy, formylamino, substituted or unsubstituted (hydroxyimino)-C₁-C₈-alkyl, substituted or unsubstituted (C₁-C₈-alkoxyimino)-C₁-C₈-alkyl, substituted or unsubstituted (C₂-C₈-alkenyloxyimino)-C₁-C₈-alkyl, substituted or unsubstituted (C₃-C₈-alkynyloxyimino)-C₁-C₈-alkyl, substituted or unsubstituted (benzyloxyimino)-C₁-C₈-alkyl, carboxy, carbamoyl, thiocarbamoyl, N-hydroxycarbamoyl, carbamate, substituted or unsubstituted C₁-C₈-alkyl, C₁-C₈-halogenoalkyl having 1 to 9 halogen atoms, substituted or unsubstituted C₂-C₈-alkenyl, C₂-C₈-halogenoalkenyl having 1 to 9 halogen atoms, substituted or unsubstituted C₂-C₈-alkynyl, C₂-C₈-halogenoalkynyl having 1 to 9 halogen atoms, substituted or unsubstituted C₁-C₈-alkoxy, C₁-C₈-halogenoalkoxy having 1 to 9 halogen atoms, substituted or unsubstituted C₁-C₈-alkylsulfanyl, C₁-C₈-halogenoalkylsulfanyl having 1 to 9 halogen atoms, substituted or unsubstituted C₁-C₈-alkylsulfinyl, C₁-C₈-halogenoalkylsulfinyl having 1 to 9 halogen atoms, substituted or unsubstituted C₁-C₈-alkylsulfonyl, C₁-C₈-halogenoalkylsulfonyl having 1 to 9 halogen atoms, substituted or unsubstituted C₁-C₈-alkylamino, substituted or unsubstituted di-(C₁-C₈-alkyl)-amino, substituted or unsubstituted C₂-C₈-alkenyloxy, C₂-C₈-halogenoalkenyloxy having 1 to 9 halogen atoms, substituted or unsubstituted C₃-C₈-alkynyloxy, C₃-C₈-halogenoalkynyloxy having 1 to 9 halogen atoms, substituted or unsubstituted C₃-C₇-cycloalkyl, C₃-C₇-halogenocycloalkyl having 1 to 9 halogen atoms, substituted or unsubstituted C₃-C₇-cycloalkyloxy, substituted or unsubstituted C₄-C₇-cycloalkenyl, C₄-C₇-halogenocycloalkenyl having 1 to 9 halogen atoms, substituted or unsubstituted (C₃-C₇-cycloalkyl)-C₁-C₈-alkyl, substituted or unsubstituted (C₃-C₇-cycloalkyl)-C₂-C₈-alkenyl, substituted or unsubstituted (C₃-C₇-cycloalkyl)-C₂-C₈-alkynyl, substituted or unsubstituted tri-(C₁-C₈-alkyl)-silyl, substituted or unsubstituted tri-(C₁-C₈-alkyl)-silyl-C₁-C₈-alkyl, substituted or unsubstituted C₁-C₈-alkylcarbonyl, C₁-C₈-halogenoalkylcarbonyl having 1 to 9 halogen atoms, substituted or unsubstituted C₁-C₈-alkylcarbonyloxy, C₁-C₈-halogenoalkylcarbonyloxy having 1 to 9 halogen atoms, substituted or unsubstituted C₁-C₈-alkylcarbonylamino, C₁-C₈-halogenoalkylcarbonylamino having 1 to 9 halogen atoms, substituted or unsubstituted C₁-C₈-alkoxycarbonyl, C₁-C₈-halogenoalkoxycarbonyl having 1 to 9 halogen atoms, substituted or unsubstituted C₁-C₈-alkyloxycarbonyloxy, C₁-C₈-halogenoalkoxycarbonyloxy having 1 to 9 halogen atoms, substituted or unsubstituted C₁-C₈-alkylcarbamoyl, substituted or unsubstituted di-C₁-C₈-alkylcarbamoyl, substituted or unsubstituted C₁-C₈-alkylaminocarbonyloxy, substituted or unsubstituted di-C₁-C₈-alkylaminocarbonyloxy, substituted or unsubstituted N—(C₁-C₈-alkyl)-hydroxycarbamoyl, substituted or unsubstituted C₁-C₈-alkoxycarbamoyl, substituted or unsubstituted N—(C₁-C₈-alkyl)-C₁-C₈-alkoxycarbamoyl, aryl optionally substituted by 1 to 6 groups Q which can be the same or different, aryl-C₁-C₈-alkyl optionally substituted by 1 to 6 groups Q which can be the same or different, aryl-C₂-C₈-alkenyl optionally substituted by 1 to 6 groups Q which can be the same or different, aryl-C₂-C₈-alkynyl optionally substituted by 1 to 6 groups Q which can be the same or different, aryloxy optionally substituted by 1 to 6 groups Q which can be the same or different, arylsulfanyl optionally substituted by 1 to 6 groups Q which can be the same or different, arylamino optionally substituted by 1 to 6 groups Q which can be the same or different, aryl-C₁-C₈-alkyloxy optionally substituted by 1 to 6 groups Q which can be the same or different, aryl-C₁-C₈-alkylsulfanyl optionally substituted by 1 to 6 groups Q which can be the same or different, aryl-C₁-C₈-alkylamino optionally substituted by 1 to 6 groups Q which can be the same or different, pyridinyl which can be substituted by 1 to 4 groups Q, pyridinyloxy which is optionally substituted by 1 to 4 groups Q, or
two substituents X together with the carbon atoms to which they are attached form a 5- or 6-membered, saturated carbocycle or saturated heterocycle, which is optionally substituted by 1 to 4 groups Q which can be the same or different,
Z¹ and Z² independently represent hydrogen, halogen, cyano, substituted or unsubstituted C₁-C₈-alkyl, C₁-C₈-halogenoalkyl having 1 to 5 halogen atoms, substituted or unsubstituted C₁-C₈-alkoxy, substituted or unsubstituted C₁-C₈-alkylsulfanyl, or substituted or unsubstituted C₁-C₈-alkoxycarbonyl,
Z³ represents hydrogen, cyano, C₁-C₈-alkoxy, unsubstituted C₃-C₇-cycloalkyl or C₃-C₇-cycloalkyl substituted by 1 to 10 substituents that can be the same or different, selected from the list consisting of halogen, cyano, C₁-C₈-alkyl, C₁-C₈-halogenoalkyl comprising 1 to 9 halogen atoms, C₁-C₈-alkoxy, C₁-C₈-halogenoalkoxy comprising 1 to 9 halogen atoms, C₁-C₈-alkoxycarbonyl, C₁-C₈-halogenoalkoxycarbonyl comprising 1 to 9 halogen atoms, C₁-C₈-alkylaminocarbonyl and di-(C₁-C₈-alkyl)-amino,
Q represents halogen, cyano, nitro, substituted or unsubstituted C₁-C₈-alkyl, C₁-C₈-halogenoalkyl having 1 to 9 halogen atoms, substituted or unsubstituted C₁-C₈-alkoxy, C₁-C₈-halogenoalkoxy having 1 to 9 halogen atoms, substituted or unsubstituted C₁-C₈-alkylsulfanyl, C₁-C₈-halogenoalkylsulfanyl having 1 to 9 halogen atoms, substituted or unsubstituted tri-(C₁-C₈-alkyl)-silyl, substituted or unsubstituted tri-(C₁-C₈-alkyl)-silyl-C₁-C₈-alkyl, substituted or unsubstituted C₁-C₈-alkoxyimino-C₁-C₈-alkyl, substituted or unsubstituted (benzyloxyimino)-C₁-C₈-alkyl, and N-oxides, metallic complexes, metalloidic complexes and optically active isomers of the compounds of formula (I),
for controlling nematodes.

Another preferred embodiment of the invention is the use of compounds of formula (IB-2) wherein A is a heterocycle of formula (A²⁶)

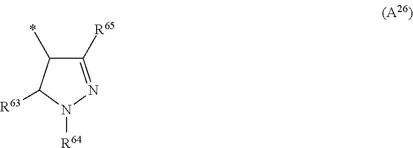

wherein
R⁶³ represents hydrogen, halogen, substituted or unsubstituted C₁-C₅-alkyl, cyano, substituted or unsubstituted C₁-C₅-alkoxy, substituted or unsubstituted C₁-C₅-alkylsulphanyl, C₁-C₅-halogenoalkyl comprising 1 to 9 halogen atoms, C₁-C₅-halogenoalkoxy comprising 1 to 9 halogen atoms, amino, substituted or unsubstituted C₁-C₅-alkylamino or di(C₁-C₅-alkyl)amino, and
R⁶⁴ represents hydrogen or substituted or unsubstituted C₁-C₅-alkyl, and
R⁶⁵ represents hydrogen, halogen, substituted or unsubstituted C₁-C₅-alkyl, substituted or unsubstituted C₃-C₅-cycloalkyl, C₁-C₅-halogenoalkyl comprising 1 to 9 halogen atoms, substituted or unsubstituted C₁-C₅-alkoxy, substituted or unsubstituted C₃-C₅-alkynyloxy or C₁-C₅-halogenoalkoxy comprising 1 to 9 halogen atoms.

Another preferred embodiment of the invention is the use of compounds of formula (IB-2) wherein A is a heterocycle of formula (A³⁰)

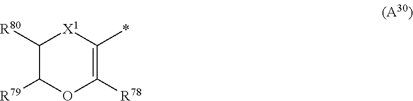

in which
X¹ represents —S—, —SO—, —SO₂— and —CH₂—, and
R⁷⁸ represents C₁-C₄-alkyl and C₁-C₄-halogenoalkyl having 1 to 5 halogen atoms, and
R⁷⁹ and R⁸⁰ independently from each other represent hydrogen and C₁-C₄-alkyl.

Another preferred embodiment of the invention is the use of compounds of formula (IB-2) wherein A is a heterocycle of formula (A³¹)

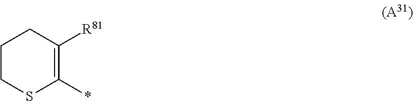

in which
R⁸¹ represents C₁-C₄-alkyl and C₁-C₄-halogenoalkyl having 1 to 5 halogen atoms.

Another preferred embodiment of the invention is the use of compounds of formula (IB-2) wherein A is a heterocycle of formula (A³²)

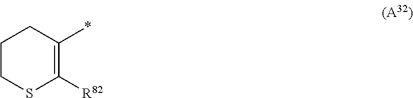

in which
R⁸² represents C₁-C₄-alkyl and C₁-C₄-halogenoalkyl having 1 to 5 halogen atoms.

Finally, it has been found that the compounds of the formula (I), including compounds of formulae (IB-1) and (IB-2) have very pronounced biological properties and are suitable in particular for controlling animal pests, especially nematodes, which are encountered in agriculture, in forests, in the protection of stored products and materials and in the hygiene sector.

Preferred substituents or ranges for the radicals shown in the compounds of the formula (I) and formula (IB-1) are elucidated below, their combination represents a preferred group (W) of compounds of formula (I) and formula (IB-1).

A represents A^{a}.
Y¹ represents halogen, nitro, C₁-C₈-alkyl, C₁-C₈-halogenoalkyl having 1 to 5 halogen atoms, C₂-C₈-alkenyl, C₂-C₈-alkynyl, C₃-C₆-cycloalkyl, C₁-C₈-alkylsulfanyl, C₁-C₈-halogenoalkylsulfanyl having 1 to 5 halogen atoms, C₁-C₈-alkoxy, C₁-C₈-halogenoalkoxy having 1 to 5 halogen atoms.
Y², Y³, Y⁴ and Y⁵ independently from each other represent hydrogen, halogen, C₁-C₈-alkyl, C₁-C₈-halogenoalkyl having 1 to 5 halogen atoms.
A also represents A⁴, wherein
R⁹ to R¹¹ independently from each other represent hydrogen, halogen, substituted or unsubstituted C₁-C₅-alkyl, amino, substituted or unsubstituted C₁-C₅-alkoxy, substituted or unsubstituted C₁-C₅-alkylsulfanyl, C₁-C₅-halogenoalkyl comprising 1 to 9 halogen atoms or C₁-C₅-halogenoalkoxy comprising 1 to 9 halogen atoms.
A also represents A⁵, wherein
R¹² and R¹³ independently from each other represent hydrogen, halogen, substituted or unsubstituted C₁-C₅-alkyl, substituted or unsubstituted C₁-C₅-alkoxy, C₁-C₅-halogenoalkyl comprising 1 to 9 halogen atoms or C₁-C₅-halogenoalkoxy comprising 1 to 9 halogen atoms, and
R¹⁴ represents hydrogen, halogen, substituted or unsubstituted C₁-C₅-alkyl, substituted or unsubstituted C₁-C₅-alkoxy, C₁-C₅-halogenoalkyl comprising 1 to 9 halogen atoms or C₁-C₅-halogenoalkoxy comprising 1 to 9 halogen atoms.
A also represents A¹², wherein
R³¹ represents hydrogen or substituted or unsubstituted C₁-C₅-alkyl, and
R³² represents hydrogen, halogen, substituted or unsubstituted C₁-C₅-alkyl or C₁-C₅-halogenoalkyl comprising 1 to 9 halogen atoms, and
R³³ represents hydrogen, halogen, nitro, substituted or unsubstituted C₁-C₅-alkyl, substituted or unsubstituted C₁-C₅-alkoxy, C₁-C₅-halogenoalkoxy comprising 1 to 9 halogen atoms or C₁-C₅-halogenoalkyl comprising 1 to 9 halogen atoms.
A also represents A¹³, wherein
R³⁴ represents hydrogen, halogen, substituted or unsubstituted C₁-C₅-alkyl, substituted or unsubstituted C₃-C₅-cycloalkyl, C₁-C₅-halogenoalkyl comprising 1 to 9 halogen atoms, substituted or unsubstituted C₁-C₅-alkoxy, substituted or unsubstituted C₃-C₅-alkynyloxy or C₁-C₅-halogenoalkoxy comprising 1 to 9 halogen atoms, and
R³⁵ represents hydrogen, halogen, substituted or unsubstituted C₁-C₅-alkyl, cyano, substituted or unsubstituted C₁-C₅-alkoxy, substituted or unsubstituted C₁-C₅-alkylsulphanyl, C₁-C₅-halogenoalkyl comprising 1 to 9 halogen atoms, C₁-C₅-halogenoalkoxy comprising 1 to 9 halogen atoms, amino, substituted or unsubstituted C₁-C₅-alkylamino or substituted or unsubstituted di(C₁-C₅-alkyl)amino, and
R³⁶ represents a hydrogen atom or substituted or unsubstituted C₁-C₅-alkyl.
A also represents A¹⁴, wherein
R³⁷ and R³⁸ independently from each other represent hydrogen, halogen, substituted or unsubstituted C₁-C₅-alkyl, C₁-C₅-halogenoalkyl comprising 1 to 9 halogen atoms, substituted or unsubstituted C₁-C₅-alkoxy or a substituted or unsubstituted C₁-C₅-alkylsulphanyl, and
R³⁹ represents hydrogen or substituted or unsubstituted C₁-C₅-alkyl.
A also represents A²⁷, in which
R⁶⁶ represents hydrogen, halogen, hydroxy, cyano, C₁-C₄-alkyl, C₁-C₄-halogenoalkyl having 1 to 5 halogen atoms, C₁-C₄-alkoxy, C₁-C₄-alkylsulfanyl, C₁-C₄-halogenoalkylsulfanyl having 1 to 5 halogen atoms and C₁-C₄-halogenoalkoxy having 1 to 5 halogen atoms, and
R⁶⁷, R⁶⁸ and R⁶⁹ independently from each other represent hydrogen, halogen, cyano, C₁-C₄-alkyl, C₁-C₄-halogenoalkyl having 1 to 5 halogen atoms, C₁-C₄-alkoxy, C₁-C₄-alkylsulfanyl, C₁-C₄-halogenoalkoxy having 1 to 5 halogen atoms, C₁-C₄-alkylsulfinyl and C₁-C₄-alkylsulfanyl.
A also represents A²⁸ in which
R⁷⁰ represents hydrogen, halogen, hydroxy, cyano, C₁-C₄-alkyl, C₁-C₄-halogenoalkyl having 1 to 5 halogen atoms, C₁-C₄-alkoxy, C₁-C₄-alkysulfanyl, C₂-C₅-alkenysulfanyll, C₁-C₄-halogenoalkylsulfanyl having 1 to 5 halogen atoms, C₁-C₄-halogenoalkoxy having 1 to 5 halogen atoms, phenyloxy (optionally substituted by halogen or C₁-C₄-alkyl) and phenylsulfanyl (optionally substituted by halogen or C₁-C₄-alkyl), and
R⁷¹, R⁷² and R⁷³ independently from each other represent hydrogen, halogen, cyano, C₁-C₄-alkyl, C₁-C₄-halogenoalkyl having 1 to 5 halogen atoms, C₁-C₄-alkoxy, C₁-C₄-alkylsulfanyl, C₁-C₄-halogenoalkoxy having 1 to 5 halogen atoms, C₁-C₄-alkylsulfinyl and C₁-C₄-alkylsulfonyl,
A also represents A³³, in which
R⁸³ represents hydrogen, halogen, C₁-C₄-alkyl and C₁-C₄-halogenoalkyl having 1 to 5 halogen atoms.
T represents oxygen.
B represents a 5- or 6-membered non-aromatic carbocycle optionally substituted by one to four C₁-C₈-alkyl groups.
n represents 1, 2 or 3.
X independently from each other X represents halogen, C₁-C₈-alkyl, C₁-C₈-halogenoalkyl having 1 to 9 halogen atoms, C₁-C₈-alkoxy, C₁-C₈-halogenoalkoxy having 1 to 9 halogen atoms, aryl optionally substituted by 1 to 6 groups Q which can be the same or different, aryloxy optionally substituted by 1 to 6 groups Q which can be the same or different, pyridinyloxy which is optionally substituted by 1 to 4 groups Q.
Q independently from each other Q represents halogen, methyl, difluoromethyl, trifluoromethyl, methoxy and trifluoromethoxy.
Z¹ and Z² independently represent hydrogen, fluorine or substituted or unsubstituted C₁-C₈-alkyl.
Z³ represents hydrogen, methoxy, cyano, cyclopropyl.

A preferred embodiment within group (W) are compounds of formula (I) and formula (IB-1) in which A represents A^{a}.

Another preferred embodiment within group (W) are compounds of formula (I) and formula (IB-1) in which A represents A⁴.

Another preferred embodiment within group (W) are compounds of formula (I) and formula (IB-1) in which A represents A⁵.

Another preferred embodiment within group (W) are compounds of formula (I) and formula (IB-1) in which A represents A¹².

Another preferred embodiment within group (W) are compounds of formula (I) and formula (IB-1) in which A represents A¹³.

Another preferred embodiment within group (W) are compounds of formula (I) and formula (IB-1) in which A represents A¹⁴.

Another preferred embodiment within group (W) are compounds of formula (I) and formula (IB-1) in which A represents A²⁷.

Another preferred embodiment within group (W) are compounds of formula (I) and formula (IB-1) in which A represents A²⁸.

Another preferred embodiment within group (W) are compounds of formula (I) and formula (IB-1) in which A represents A³³.

Further preferred embodiments within group (W) are groups (W1) to (W6).

(W1) Compounds of formula (I) and formula (IB-1) wherein A represents A^{a} in which Y¹ represents a radical selected from the group consisting of halogen, methyl, difluoromethyl, trifluoromethyl, methoxy, difluoromethoxy and trifluoromethoxy, Y², Y³ and Y⁴ represent hydrogen and Y⁵ represents hydrogen or fluorine.
(W2) Compounds of formula (I) and formula (IB-1) wherein A represents A⁴ in which wherein R⁹ and R¹⁰ represent hydrogen and R¹¹ represents halogen, meth, C₁-fluoroalkyl comprising 1 to 3 fluorine atoms.
(W3) Compounds of formula (I) and formula (IB-1) wherein A represents A⁵ in which R¹² and R¹⁴ independently from each other represent hydrogen, halogen, methyl, C₁-fluoroalkyl comprising 1 to 3 fluorine atoms that can be the same or different, and
R¹³ represents hydrogen.
(W4) Compounds of formula (I) and formula (IB-1) wherein A represents A¹³ in which R³⁴ represents hydrogen, halogen, C₁-C₂-alkyl, cyclopropyl, C₁-C₂-halogenoalkyl comprising 1 to 9 halogen atoms, C₁-C₂-alkoxy, or C₁-C₂-halogenoalkoxy comprising 1 to 5 halogen atoms, and
R³⁵ represents hydrogen, halogen, methyl, ethyl, C₁-halogenoalkyl comprising 1 to 3 halogen atoms, C₁-halogenoalkoxy comprising 1 to 3 halogen atoms, and
R³⁶ represents methyl.
(W5) Compounds of formula (I) and formula (IB-1) wherein A represents A²⁸ in which R⁷⁰ represents hydrogen, halogen, cyano, methyl, ethyl, difluoromethyl, trifluoromethyl, methoxy, trifluromethylsulfanyl, trifluoromethoxy, and
R⁷¹, R⁷² represent hydrogen and R⁷³ represents hydrogen, halogen, cyano, methyl, ethyl, difluoromethyl, trifluoromethyl, methoxy, trifluromethylsulfanyl, trifluoromethoxy.
(W6) Compounds of formula (I) and formula (IB-1) wherein A represents A³³ in which R⁸³ represents hydrogen, halogen, methyl, ethyl, difluoromethyl and trifluoromethyl.

Particularly preferred substituents or ranges for the radicals shown in the compounds of the formula (I) and formula (IB-1) are elucidated below, their combination represents a particularly preferred group (U) of compounds of formula (I) and formula (IB-1).

A represents A^{a}.
Y¹ represents a radical selected from the group consisting of halogen, methyl, difluoromethyl, trifluoromethyl, methoxy, difluoromethoxy and trifluoromethoxy.
Y², Y³ and Y⁴ represent hydrogen.
Y⁵ represents hydrogen or fluorine.
A also represents A⁴, wherein
R⁹ to R¹¹ independently from each other represent hydrogen, halogen, C₁-C₅-alkyl C₁-C₅-alkoxy C₁-C₅-alkylsulfanyl, C₁-C₅-halogenoalkyl comprising 1 to 9 halogen atoms or C₁-C₅-halogenoalkoxy comprising 1 to 9 halogen atoms.
A also represents A⁵, wherein
R¹² and R¹³ independently from each other represent hydrogen, halogen, C₁-C₅-alkyl, C₁-C₅-alkoxy, C₁-C₅-halogenoalkyl comprising 1 to 9 halogen atoms or C₁-C₅-halogenoalkoxy comprising 1 to 9 halogen atoms, and
R¹⁴ represents hydrogen, halogen, C₁-C₅-alkyl, C₁-C₅-alkoxy, C₁-C₅-halogenoalkyl comprising 1 to 9 halogen atoms or C₁-C₅-halogenoalkoxy comprising 1 to 9 halogen atoms.
A also represents A¹³, wherein
R³⁴ represents hydrogen, halogen, C₁-C₅-alkyl, C₃-C₅-cycloalkyl, C₁-C₅-halogenoalkyl comprising 1 to 9 halogen atoms, C₁-C₅-alkoxy, C₂-C₅-alkynyloxy or C₁-C₅-halogenoalkoxy comprising 1 to 9 halogen atoms, and
R³⁵ represents hydrogen, halogen, C₁-C₅-alkyl, cyano, C₁-C₅-alkoxy, C₁-C₅-alkylsulphanyl, C₁-C₅-halogenoalkyl comprising 1 to 9 halogen atoms, C₁-C₅-halogenoalkoxy comprising 1 to 9 halogen atoms, amino, C₁-C₅-alkylamino or di(C₁-C₅-alkyl)amino, and
R³⁶ represents a hydrogen atom or C₁-C₅-alkyl.
A also represents A²⁸, in which
R⁷⁰ represents hydrogen, halogen, hydroxy, cyano, C₁-C₄-alkyl, C₁-C₄-halogenoalkyl having 1 to 5 halogen atoms, C₁-C₄-alkoxy, C₁-C₅-alkylsulfanyl, C₂-C₅-alkenylsulfanyl, C₁-C₄-halogenoalkylsulfanyl having 1 to 5 halogen atoms, C₁-C₄-halogenoalkoxy having 1 to 5 halogen atoms, phenyloxy (optionally substituted by halogen or C₁-C₄-alkyl) and phenylsulfanyl (optionally substituted by halogen or C₁-C₄-alkyl), and
R⁷¹, R⁷² and R⁷³ independently from each other represent hydrogen, halogen, cyano, C₁-C₄-alkyl, C₁-C₄-halogenoalkyl having 1 to 5 halogen atoms, C₁-C₄-alkoxy, C₁-C₄-alkylsulfanyl, C₁-C₄-halogenoalkoxy having 1 to 5 halogen atoms, C₁-C₄-alkylsulfinyl and C₁-C₄-alkylsulfonyl.
A also represents A³³, in which
R⁸³ represents hydrogen, halogen, C₁-C₄-alkyl and C₁-C₄-halogenoalkyl having 1 to 5 halogen atoms.
T represents oxygen.
B represents a 4- or 5-membered non-aromatic carbocycle optionally substituted by one to four C₁-C₈-alkyl groups.
n represents 1, 2 or 3.
X independently from each other X represents halogen, C₁-C₃-alkyl, C₁-C₂-halogenoalkyl having 1 to 2 halogen atoms, C₁-C₄-alkoxy, C₁-C₂-halogenoalkoxy having 1 to 5 halogen atoms.
Z¹ and Z² independently represent hydrogen, fluorine or substituted or unsubstituted C₁-C₈-alkyl.
Z³ represents hydrogen.

Preferred embodiments within group (U) are groups (U1) to (U6).

(U1) Compounds of formula (I) and formula (IB-1) wherein A represents A^{a} in which Y¹ represents a radical selected from the group consisting of halogen, methyl, difluoromethyl, trifluoromethyl, methoxy, difluoromethoxy and trifluoromethoxy, Y², Y³ and Y⁴ represent hydrogen and Y⁵ represents hydrogen or fluorine.
(U2) Compounds of formula (I) and formula (IB-1) wherein A represents A⁴ in which R⁹ and R¹⁰ represent hydrogen and R¹¹ represents halogen, methyl, C₁-fluoroalkyl comprising 1 to 3 fluorine atoms.
(U3) Compounds of formula (I) and formula (IB-1) wherein A represents A⁵ in which R¹² and R¹⁴ independently from each other represent hydrogen, halogen, methyl, methoxy, C₁-fluoroalkyl comprising 1 to 3 fluorine atoms that can be the same or different, and
R¹³ represents hydrogen.
(U4) Compounds of formula (I) and formula (IB-1) wherein A represents A¹³ in which R³⁴ represents hydrogen, halogen, C₁-C₂-alkyl, cyclopropyl, C₁-C₂-halogenoalkyl comprising 1 to 5 halogen atoms that can be the same or different, C₁-C₂-alkoxy, or C₁-C₂-halogenoalkoxy comprising 1 to 5 halogen atoms that can be the same or different, and
R³⁵ represents hydrogen, halogen, methyl, ethyl, C₁-halogenoalkyl comprising 1 to 3 halogen atoms, C₁-halogenoalkoxy comprising 1 to 3 halogen atoms, and
R³⁶ represents methyl.
(U5) Compounds of formula (I) and formula (IB-1) wherein A represents A²⁸ in which R⁷⁰ represents hydrogen, halogen, cyano, methyl, ethyl, difluoromethyl, trifluoromethyl, methoxy, trifluromethylsulfanyl, trifluoromethoxy, and
R⁷¹, R⁷² represent hydrogen and R⁷³ represents hydrogen, halogen, cyano, methyl, ethyl, difluoromethyl, trifluoromethyl, methoxy, trifluromethylsulfanyl, trifluoromethoxy.
(U6) Compounds of formula (I) and formula (IB-1) wherein A represents A³³ in which R⁸³ represents hydrogen, halogen, methyl, ethyl, difluoromethyl and trifluoromethyl.

A particularly preferred embodiment of the present invention are compounds within groups (W) and within groups (W1) to (W6) in which B represents cyclobutyl optionally substituted by one to four methyl groups.

Another particularly preferred embodiment of the present invention are compounds within groups (U) and within groups (U1) to (U6) in which B represents cyclobutyl optionally substituted by one to four methyl groups.

The above mentioned preferences with regard to the substituents of the compounds according to the invention can be combined in various manners. These combinations of preferred features thus provide preferred sub-classes of compounds according to the invention.

The present invention is also directed to compounds of formula (I)

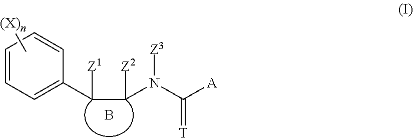

wherein

A represents phenyl of formula A^{a}

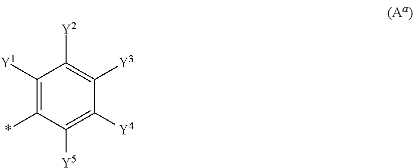

wherein
* indicates the bond which connects A^{a} to the C=T moiety of the compounds of formula (I),
Y¹ represents halogen, nitro, SH, SF₅, CHO, OCHO, NHCHO, cyano, C₁-C₈-alkyl, C₁-C₈-halogenoalkyl having 1 to 5 halogen atoms, C₂-C₈-alkenyl, C₂-C₈-alkynyl, C₃-C₆-cycloalkyl, C₃-C₆-halogenocycloalkyl having 1 to 9 halogen atoms, C₁-C₈-alkylsulfanyl, —C₁-C₈-halogenoalkylsulfanyl having 1 to 5 halogen atoms, C₁-C₈-alkoxy, C₁-C₈-halogenoalkoxy having 1 to 5 halogen atoms, C₁-C₈-alkoxy-C₁-C₈-alkyl, C₂-C₈-alkenyloxy-C₁-C₈-alkyl, C₃-C₈-alkynyloxy-C₁-C₈-alkyl, C₂-C₈-alkenyloxy, C₃-C₈-alkynyloxy, C₁-C₈-alkoxycarbonyl, C₁-C₈-halogenoalkoxycarbonyl having 1 to 5 halogen atoms, C₁-C₈-alkylcarbonyloxy, C₁-C₈-halogenoalkylcarbonyloxy having 1 to 5 halogen atoms, C₁-C₈-alkylsulfinyl, C₁-C₈-halogenoalkylsulfinyl having 1 to 5 halogen atoms, C₁-C₈-alkylsulfonyl, C₁-C₈-halogenoalkylsulfonyl having 1 to 5 halogen atoms, C₁-C₈-alkylsulfonamide, substituted or unsubstituted tri-(C₁-C₈)-alkylsilyl, substituted or unsubstituted aryl and substituted or unsubstituted aryloxy;
Y², Y³, Y⁴ and Y⁵ independently from each other represent hydrogen or Y¹; or
A represents a carbo-linked heterocyclyl group optionally substituted by 1 to five groups R, wherein
R independently from each other R represents hydrogen, halogen, nitro, cyano, hydroxy, amino, sulfanyl, pentafluoro-λ⁶-sulfanyl, substituted or unsubstituted (C₁-C₈-alkoxyimino)-C₁-C₈-alkyl, substituted or unsubstituted (benzyloxyimino)-C₁-C₈-alkyl, substituted or unsubstituted C₁-C₈-alkyl, C₁-C₈-halogenoalkyl having 1 to 5 halogen atoms, substituted or unsubstituted C₂-C₈-alkenyl, C₂-C₈-halogenoalkenyl having 1 to 5 halogen atoms, substituted or unsubstituted C₂-C₈-alkynyl, C₂-C₈-halogenoalkynyl having 1 to 5 halogen atoms, substituted or unsubstituted C₁-C₈-alkoxy, C₁-C₈-halogenoalkoxy having 1 to 5 halogen atoms, substituted or unsubstituted C₁-C₈-alkylsulfanyl, C₁-C₈-halogenoalkylsulfanyl having 1 to 5 halogen atoms, substituted or unsubstituted C₁-C₈-alkylsulfinyl, C₁-C₈-halogenoalkylsulfinyl having 1 to 5 halogen atoms, substituted or unsubstituted C₁-C₈-alkylsulfonyl, C₁-C₈-halogenoalkylsulfonyl having 1 to 5 halogen atoms, substituted or unsubstituted C₁-C₈-alkylamino, substituted or unsubstituted di-C₁-C₈-alkylamino, substituted or unsubstituted C₂-C₈-alkenyloxy, substituted or unsubstituted C₃-C₈-alkynyloxy, substituted or unsubstituted C₃-C₇-cycloalkyl, C₃-C₇-halogenocycloalkyl having 1 to 5 halogen atoms, substituted or unsubstituted tri-(C₁-C₈-alkyl)-silyl, substituted or unsubstituted C₁-C₈-alkylcarbonyl, C₁-C₈-halogenoalkylcarbonyl having 1 to 5 halogen atoms, substituted or unsubstituted C₁-C₈-alkoxycarbonyl, C₁-C₈-halogenoalkoxycarbonyl having 1 to 5 halogen atoms, substituted or unsubstituted C₁-C₈-alkylcarbamoyl, substituted or unsubstituted di-(C₁-C₈)-alkylcarbamoyl, phenoxy, phenylsulfanyl, phenylamino, benzyloxy, benzylsulfanyl, or benzylamino,
T represents sulphur,
B represents a 3-, 4-, 5-, 6- or 7-membered non-aromatic carbocycle optionally substituted by substituents selected from the group consisting of one to four C₁-C₈-alkyl groups and one to four halogen atoms,
n represents 0, 1, 2, 3, 4 or 5,
X independently from each other X represents halogen, nitro, cyano, isonitrile, hydroxy, amino, sulfanyl, pentafluoro-λ⁶-sulfanyl, formyl, formyloxy, formylamino, substituted or unsubstituted (hydroxyimino)-C₁-C₈-alkyl, substituted or unsubstituted (C₁-C₈-alkoxyimino)-C₁-C₈-alkyl, substituted or unsubstituted (C₂-C₈-alkenyloxyimino)-C₁-C₈-alkyl, substituted or unsubstituted (C₃-C₈-alkynyloxyimino)-C₁-C₈-alkyl, substituted or unsubstituted (benzyloxyimino)-C₁-C₈-alkyl, carboxy, carbamoyl, thiocarbamoyl, N-hydroxycarbamoyl, carbamate, substituted or unsubstituted C₁-C₈-alkyl, C₁-C₈-halogenoalkyl having 1 to 9 halogen atoms, substituted or unsubstituted C₂-C₈-alkenyl, C₂-C₈-halogenoalkenyl having 1 to 9 halogen atoms, substituted or unsubstituted C₂-C₈-alkynyl, C₂-C₈-halogenoalkynyl having 1 to 9 halogen atoms, substituted or unsubstituted C₁-C₈-alkoxy, C₁-C₈-halogenoalkoxy having 1 to 9 halogen atoms, substituted or unsubstituted C₁-C₈-alkylsulfanyl, C₁-C₈-halogenoalkylsulfanyl having 1 to 9 halogen atoms, substituted or unsubstituted C₁-C₈-alkylsulfinyl, C₁-C₈-halogenoalkylsulfinyl having 1 to 9 halogen atoms, substituted or unsubstituted C₁-C₈-alkylsulfonyl, C₁-C₈-halogenoalkylsulfonyl having 1 to 9 halogen atoms, substituted or unsubstituted C₁-C₈-alkylamino, substituted or unsubstituted di-(C₁-C₈-alkyl)-amino, substituted or unsubstituted C₂-C₈-alkenyloxy, C₂-C₈-halogenoalkenyloxy having 1 to 9 halogen atoms, substituted or unsubstituted C₃-C₈-alkynyloxy, C₃-C₈-halogenoalkynyloxy having 1 to 9 halogen atoms, substituted or unsubstituted C₃-C₇-cycloalkyl, C₃-C₇-halogenocycloalkyl having 1 to 9 halogen atoms, substituted or unsubstituted C₃-C₇-cycloalkyloxy, substituted or unsubstituted C₄-C₇-cycloalkenyl, C₄-C₇-halogenocycloalkenyl having 1 to 9 halogen atoms, substituted or unsubstituted (C₃-C₇-cycloalkyl)-C₁-C₈-alkyl, substituted or unsubstituted (C₃-C₇-cycloalkyl)-C₂-C₈-alkenyl, substituted or unsubstituted (C₃-C₇-cycloalkyl)-C₂-C₈-alkynyl, substituted or unsubstituted tri-(C₁-C₈-alkyl)-silyl, substituted or unsubstituted tri-(C₁-C₈-alkyl)-silyl-C₁-C₈-alkyl, substituted or unsubstituted C₁-C₈-alkylcarbonyl, C₁-C₈-halogenoalkylcarbonyl having 1 to 9 halogen atoms, substituted or unsubstituted C₁-C₈-alkylcarbonyloxy, C₁-C₈-halogenoalkylcarbonyloxy having 1 to 9 halogen atoms, substituted or unsubstituted C₁-C₈-alkylcarbonylamino, C₁-C₈-halogenoalkylcarbonylamino having 1 to 9 halogen atoms, substituted or unsubstituted C₁-C₈-alkoxycarbonyl, C₁-C₈-halogenoalkoxycarbonyl having 1 to 9 halogen atoms, substituted or unsubstituted C₁-C₈-alkyloxycarbonyloxy, C₁-C₈-halogenoalkoxycarbonyloxy having 1 to 9 halogen atoms, substituted or unsubstituted C₁-C₈-alkylcarbamoyl, substituted or unsubstituted di-C₁-C₈-alkylcarbamoyl, substituted or unsubstituted C₁-C₈-alkylaminocarbonyloxy, substituted or unsubstituted di-C₁-C₈-alkylaminocarbonyloxy, substituted or unsubstituted N—(C₁-C₈-alkyl)-hydroxycarbamoyl, substituted or unsubstituted C₁-C₈-alkoxycarbamoyl, substituted or unsubstituted N—(C₁-C₈-alkyl)-C₁-C₈-alkoxycarbamoyl, aryl optionally substituted by 1 to 6 groups Q which can be the same or different, aryl-C₁-C₈-alkyl optionally substituted by 1 to 6 groups Q which can be the same or different, aryl-C₂-C₈-alkenyl optionally substituted by 1 to 6 groups Q which can be the same or different, aryl-C₂-C₈-alkynyl optionally substituted by 1 to 6 groups Q which can be the same or different, aryloxy optionally substituted by 1 to 6 groups Q which can be the same or different, arylsulfanyl optionally substituted by 1 to 6 groups Q which can be the same or different, arylamino optionally substituted by 1 to 6 groups Q which can be the same or different, aryl-C₁-C₈-alkyloxy optionally substituted by 1 to 6 groups Q which can be the same or different, aryl-C₁-C₈-alkylsulfanyl optionally substituted by 1 to 6 groups Q which can be the same or different, aryl-C₁-C₈-alkylamino optionally substituted by 1 to 6 groups Q which can be the same or different, pyridinyl which can be substituted by 1 to 4 groups Q, pyridinyloxy which is optionally substituted by 1 to 4 groups Q, or
two substituents X together with the carbon atoms to which they are attached form a 5- or 6-membered, saturated carbocycle or saturated heterocycle, which is optionally substituted by 1 to 4 groups Q which can be the same or different,
Z¹ and Z² independently represent hydrogen, halogen, cyano, substituted or unsubstituted C₁-C₈-alkyl, C₁-C₈-halogenoalkyl having 1 to 5 halogen atoms, substituted or unsubstituted C₁-C₈-alkoxy, substituted or unsubstituted C₁-C₈-alkylsulfanyl, or substituted or unsubstituted C₁-C₈-alkoxycarbonyl,
Z³ represents hydrogen, cyano, C₁-C₈-alkoxy, unsubstituted C₃-C₇-cycloalkyl or C₃-C₇-cycloalkyl substituted by 1 to 10 substituents that can be the same or different, selected from the list consisting of halogen, cyano, C₁-C₈-alkyl, C₁-C₈-halogenoalkyl comprising 1 to 9 halogen atoms, C₁-C₈-alkoxy, C₁-C₈-halogenoalkoxy comprising 1 to 9 halogen atoms, C₁-C₈-alkoxycarbonyl, C₁-C₈-halogenoalkoxycarbonyl comprising 1 to 9 halogen atoms, C₁-C₈-alkylaminocarbonyl and di-(C₁-C₈-alkyl)-amino,
Q represents halogen, cyano, nitro, substituted or unsubstituted C₁-C₈-alkyl, C₁-C₈-halogenoalkyl having 1 to 9 halogen atoms, substituted or unsubstituted C₁-C₈-alkoxy, C₁-C₈-halogenoalkoxy having 1 to 9 halogen atoms, substituted or unsubstituted C₁-C₈-alkylsulfanyl, C₁-C₈-halogenoalkylsulfanyl having 1 to 9 halogen atoms, substituted or unsubstituted tri-(C₁-C₈-alkyl)-silyl, substituted or unsubstituted tri-(C₁-C₈-alkyl)-silyl-C₁-C₅-alkyl, substituted or unsubstituted C₁-C₈-alkoxyimino-C₁-C₈-alkyl, substituted or unsubstituted (benzyloxyimino)-C₁-C₈-alkyl,
and N-oxides, metallic complexes, metalloidic complexes and optically active isomers of the compounds of formula (I).

The present invention is also directed to novel compounds of formula (IB-1-1)

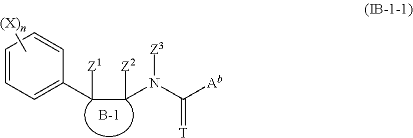

in which T, Z¹, Z² and X have the meanings mentioned above for the compounds of formula (IB-1),
B-1 represents a 5-, 6- or 7-membered non-aromatic carbocycle optionally substituted by substituents selected from the group consisting of one to four C₁-C₈-alkyl groups and one to four halogen atoms, and
Z³ represents hydrogen,
A^{b} represents A^{a} and represents A¹ to A²⁶ as defined above, provided that
n does not represent 0 if B-1 represents an unsubstituted 6-membered non-aromatic carbocycle and provided that X does not represent alkoxy if A^{b} represents A^{a}.

The present invention is further directed to novel compounds of formula (IB-2-1)

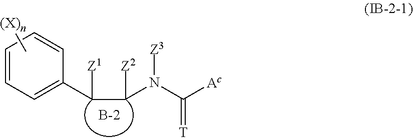

in which T, Z¹, Z², X and n have the meanings mentioned above for the compounds of formula (I),
Z³ represents hydrogen,
B-2 represents a 3- or 4-membered non-aromatic carbocycle optionally substituted by substituents selected from the group consisting of one to four C₁-C₈-alkyl groups and one to four halogen atoms, and
A^{c} represents A²⁶.

The above in connection with the compounds of formula (I) mentioned preferred definitions and embodiments are also valid for the novel compounds of formula (IB-1-1), provided that n does not represent 0 if B-1 represents an unsubstituted 6-membered non-aromatic carbocycle and provided that X does not represent alkoxy if A^{b} represents A^{a}.

The above in connection with the compounds of formula (I) mentioned preferred definitions and embodiments are also valid for the novel compounds of formula (IB-2-1).

The present invention also relates to a process for the preparation of the compounds of formula (IB-1).

Thus, according to a further aspect of the present invention there is provided a process P1 for the preparation of a compound of formula (IB-1-1) as herein-defined wherein T represents O which comprises reaction of an amine of formula (II-1) or one of its salts:

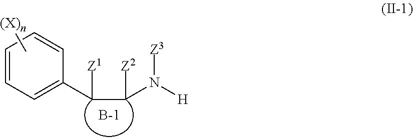

in which Z¹, Z², X and n have the meanings mentioned above for the compounds of formula (IB-1-1),
B-1 represents a 5-, 6- or 7-membered non-aromatic carbocycle optionally substituted by substituents selected from the group consisting of one to four C₁-C₈-alkyl groups and one to four halogen atoms, and
Z³ represents hydrogen,
with a carboxylic acid derivative of formula (III-1):

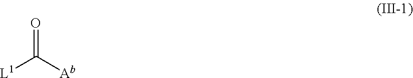

wherein A^{b} represents A^{a} and represents A¹ to A²⁶ as defined above, L¹ represents a leaving group selected from the group consisting of halogen, OH, —OR^{a}, —OC(═O)R^{a}, R^{a} being substituted or unsubstituted C₁-C₆-alkyl, a substituted or unsubstituted C₁-C₆-haloalkyl, benzyl, 4-methoxybenzyl or pentafluorophenyl, or a group of formula O—C(═O)A^{b}; in the presence of a catalyst and in the presence of a condensing agent in case L¹ represents OH, and in the presence of an acid binder in case L¹ represents a halogen atom.

Amine derivatives of formula (II-1) are known or can be prepared by known processes such as reductive amination of aldehydes or ketones (Organic Reactions (Hoboken, N.J., United States) (2002), 59,), or reduction of oximes (Journal of Medicinal Chemistry (1984), 27(9), 1108), or nucleophilic substitution of a halogen, mesylate or tosylate (Journal of Medicinal Chemistry (2002), 45, 3887).

Carboxylic acid derivatives of formula (III-1) are known or can be prepared by known processes.

In case L¹ represents OH, process P1 according to the present invention is conducted in the presence of condensing agent. Suitable condensing agents may be selected in the non-limited list consisting of acid halide former, such as phosgene, phosphorous tribromide, phosphorous trichloride, phosphorous pentachloride, phosphorous trichloride oxide or thionyl chloride; anhydride former, such as ethyl chloroformate, methyl chloroformate, isopropyl chloroformate, isobutyl chloroformate or methanesulfonyl chloride; carbodiimides, such as N,N′-dicyclohexylcarbodiimide (DCC) or other customary condensing agents, such as phosphorous pentoxide, polyphosphoric acid, N,N′-carbonyl-diimidazole, 2-ethoxy-N-ethoxycarbonyl-1,2-dihydroquinoline (EEDQ), triphenylphosphine/tetrachloro-methane, 4-(4,6-dimethoxy[1.3.5]-triazin-2-yl)-4-methylmorpholinium chloride hydrate, bromotripyrrolidinophosphonium-hexafluorophosphate or propanephosphonic anhydride (T3P).

Process P1 according to the present invention is conducted in the presence of a catalyst. Suitable catalyst may be selected from the list consisting of N,N-dimethylpyridin-4-amine, 1-hydroxy-benzotriazole or N,N-dimethylformamide.

In case L¹ represents a halogen atom, process P1 according to the present invention is conducted in the presence of an acid binder. Suitable acid binders are all inorganic and organic bases that are customary for such reactions. Preference is given to using alkaline earth metal, alkali metal hydride, alkali metal hydroxides or alkali metal alkoxides, such as sodium hydroxide, sodium hydride, calcium hydroxide, potassium hydroxide, potassium tert-butoxide or other ammonium hydroxide, alkali metal carbonates, such as caesium carbonate, sodium carbonate, potassium carbonate, potassium bicarbonate, sodium bicarbonate, alkali metal or alkaline earth metal acetates, such as sodium acetate, potassium acetate, calcium acetate and also tertiary amines, such as trimethylamine, triethylamine, diisopropylethylamine, tributylamine, N,N-dimethylaniline, pyridine, N-methylpiperidine, N,N-dimethylpyridin-4-amine, diazabicyclooctane (DABCO), diazabicyclo-nonene (DBN) or diazabicycloundecene (DBU).

It is also possible to work in the absence of an additional condensing agent or to employ an excess of the amine component, so that it simultaneously acts as acid binder agent.

Suitable solvents for carrying out process P1 according to the invention can be customary inert organic solvents. Preference is given to using optionally halogenated aliphatic, alicyclic or aromatic hydrocarbons, such as petroleum ether, hexane, heptane, cyclohexane, methylcyclohexane, benzene, toluene, xylene or decalin; chlorobenzene, dichlorobenzene, dichloromethane, chloroform, carbon tetrachloride, dichlorethane or trichlorethane; ethers, such as diethyl ether, diisopropyl ether, methyl t-butyl ether, methyl t-amyl ether, dioxane, tetrahydrofuran, 1,2-dimethoxyethane, 1,2-diethoxyethane or anisole; nitriles, such as acetonitrile, propionitrile, n- or i-butyronitrile or benzonitrile; amides, such as N,N-dimethylformamide, N,N-dimethylacetamide, N-methylformanilide, N-methylpyrrolidone, or hexamethylphosphoric triamide; alcohols such as methanol, ethanol, propanol, iso-propanol; esters, such as methyl acetate or ethyl acetate, sulfoxides, such as dimethyl sulfoxide, or sulfones, such as sulfolane.

When carrying out process P1 according to the invention, the amine derivative of formula (II) can be employed as its salt, such as chlorhydrate or any other convenient salt.

When carrying out process P1 according to the invention, 1 mole or an excess of the amine derivative of formula (II) and from 1 to 3 moles of the acid binder can be employed per mole of the reagent of formula (III).

It is also possible to employ the reaction components in other ratios. Work-up is carried out by known methods.

According to a further aspect according to the invention, there is provided a second process P2 for the preparation of a compound of formula (IB-1-1) wherein T represents S, starting from a compound of formula (IB-1-1) wherein T represents O and illustrated according to the following reaction scheme:

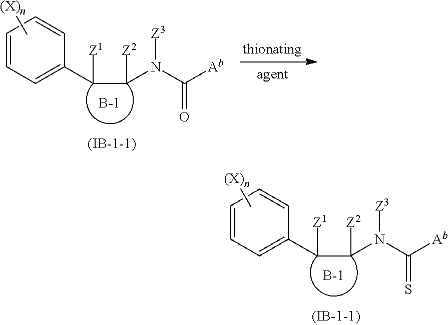

### Process P2

in which Z¹, Z², X and n have the meanings mentioned above for the compounds of formula (IB-1-1),
B-1 represents a 5-, 6- or 7-membered non-aromatic carbocycle optionally substituted by substituents selected from the group consisting of one to four C₁-C₈-alkyl groups and one to four halogen atoms, and
Z³ represents hydrogen,
A^{b} represents A^{a} and represents A¹ to A²⁶ as defined above.

Process P2 according to the invention is performed in the presence of a thionating agent.

Amide derivatives of formula (IB-1-1) wherein T represents O can be prepared according to processes P1.

Suitable thionating agents can be sulfur (S), sulfhydric acid (H₂S), sodium sulfide (Na₂S), sodium hydrosulfide (NaHS), boron trisulfide (B₂S₃), bis(diethylaluminium) sulfide ((AlEt₂)₂S), ammonium sulfide ((NH₄)₂S), phosphorous pentasulfide (P₂S₅), Lawesson's reagent (2,4-bis(4-methoxyphenyl)-1,2,3,4-dithiadiphosphetane 2,4-disulfide) or a polymer-supported thionating reagent as described in Journal of the Chemical Society, Perkin 1 (2001), 358, in the optionally presence of a catalytic or stoichiometric or excess amount, quantity of a base such as an inorganic and organic base. Preference is given to using alkali metal carbonates, such as sodium carbonate, potassium carbonate, potassium bicarbonate, sodium bicarbonate; heterocyclic aromatic bases, such as pyridine, picoline, lutidine, collidine; and also tertiary amines, such as trimethylamine, triethylamine, tributylamine, N,N-dimethylaniline, N,N-dimethylpyridin-4-amine or N-methyl-piperidine.

Suitable solvents for carrying out process P2 according to the invention can be customary inert organic solvents. Preference is given to using optionally halogenated aliphatic, alicyclic or aromatic hydrocarbons, such as petroleum ether, hexane, heptane, cyclohexane, methylcyclohexane, benzene, toluene, xylene or decalin, chlorobenzene, dichlorobenzene, dichloromethane, chloroform, carbon tetrachloride, dichlorethane or trichlorethane, ethers, such as diethyl ether, diisopropyl ether, methyl t-butyl ether, methyl t-amyl ether, dioxane, tetrahydrofuran, 1,2-dimethoxyethane or 1,2-diethoxyethane, nitriles, such as acetonitrile, propionitrile, n- or i-butyronitrile or benzonitrile, sulfurous solvents, such as sulfolane or carbon disulfide.

When carrying out process P2 according to the invention, 1 mole or an excess of the sulfur equivalent of the thionating agent and from 1 to 3 moles of the base can be employed per mole of the amide reactant.

It is also possible to employ the reaction components in other ratios. Work-up is carried out by known methods.

Processes P1 and P2 according to the invention are generally carried out under atmospheric pressure. It is also possible to operate under elevated or reduced pressure.

When carrying out processes P1 and P2 according to the invention, the reaction temperatures can be varied within a relatively wide range. In general, these processes are carried out at temperatures from 0° C. to 200° C., preferably from 10° C. to 160° C. A way to control the temperature for the processes according to the invention is to use micro-wave technology.

In general, the reaction mixture is concentrated under reduced pressure. The residue that remains can be freed by known methods, such as chromatography or crystallization, from any impurities that can still be present.

The present invention also relates to a process for the preparation of the compounds of formula (IB-2-1).

Thus, according to a further aspect of the present invention there is provided a process P′1 for the preparation of a compound of formula (IB-2-1) as herein-defined and wherein T represents O and that comprises reaction of an amine of formula (II-2) or one of its salts:

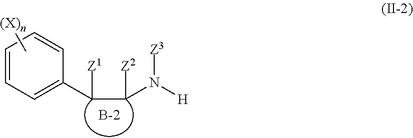

in which T, Z¹, Z², X and n have the meanings mentioned above for the compounds of formula (IB-2-1),
Z³ represents hydrogen,
B-2 represents a 3- or 4-membered non-aromatic carbocycle optionally substituted by substituents selected from the group consisting of one to four C₁-C₈-alkyl groups and one to four halogen atoms, with a carboxylic acid derivative of formula (III-2):

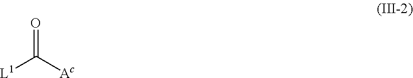

wherein A^{c} represents A²⁶,
L¹ represents a leaving group selected in the list consisting of a halogen atom, OH, —OR^{a}, —OC(═O)R^{a}, R^{a} being substituted or unsubstituted C₁-C₆-alkyl, substituted or unsubstituted C₁-C₆-haloalkyl, benzyl, 4-methoxybenzyl or pentafluorophenyl, or a group of formula O—C(═O)A^{b}; in the presence of a catalyst and in the presence of a condensing agent in case L¹ represents OH, and in the presence of an acid binder in case L¹ represents a halogen atom.

Amine derivatives of formula (II-2) are known or can be prepared by known processes.

Carboxylic acid derivatives of formula (III-2) are known or can be prepared by known processes.

In case L¹ represents OH, process P′1 according to the present invention is conducted in the presence of condensing agent. Suitable condensing agent may be selected in the non-limited list consisting of acid halide former, such as phosgene, phosphorous tribromide, phosphorous trichloride, phosphorous pentachloride, phosphorous trichloride oxide or thionyl chloride; anhydride former, such as ethyl chloroformate, methyl chloroformate, isopropyl chloroformate, isobutyl chloroformate or methanesulfonyl chloride; carbodiimides, such as N,N′-dicyclohexylcarbodiimide (DCC) or other customary condensing agents, such as phosphorous pentoxide, polyphosphoric acid, N,N′-carbonyl-diimidazole, 2-ethoxy-N-ethoxycarbonyl-1,2-dihydroquinoline (EEDQ), triphenylphosphine/tetrachloro-methane, 4-(4,6-dimethoxy[1.3.5]-triazin-2-yl)-4-methylmorpholinium chloride hydrate, bromo-tripyrrolidinophosphoniumhexafluorophosphate or propanephosphonic anhydride (T3P).

Process P′1 according to the present invention is conducted in the presence of a catalyst. Suitable catalyst may be selected in the list consisting of N,N-dimethylpyridin-4-amine, 1-hydroxy-benzotriazole or N,N-dimethylformamide.

In case L¹ represents a halogen atom, process P′1 according to the present invention is conducted in the presence of an acid binder. Suitable acid binders for carrying out process P1 according to the invention are in each case all inorganic and organic bases that are customary for such reactions. Preference is given to using alkaline earth metal, alkali metal hydride, alkali metal hydroxides or alkali metal alkoxides, such as sodium hydroxide, sodium hydride, calcium hydroxide, potassium hydroxide, potassium tert-butoxide or other ammonium hydroxide, alkali metal carbonates, such as caesium carbonate, sodium carbonate, potassium carbonate, potassium bicarbonate, sodium bicarbonate, alkali metal or alkaline earth metal acetates, such as sodium acetate, potassium acetate, calcium acetate and also tertiary amines, such as trimethylamine, triethylamine, diisopropylethylamine, tributylamine, N,N-dimethylaniline, pyridine, N-methylpiperidine, N,N-dimethylpyridin-4-amine, diazabicyclooctane (DABCO), diazabicyclo-nonene (DBN) or diazabicycloundecene (DBU).

It is also possible to work in the absence of an additional condensing agent or to employ an excess of the amine component, so that it simultaneously acts as acid binder agent.

Suitable solvents for carrying out process P′1 according to the invention can be customary inert organic solvents. Preference is given to using optionally halogenated aliphatic, alicyclic or aromatic hydrocarbons, such as petroleum ether, hexane, heptane, cyclohexane, methylcyclohexane, benzene, toluene, xylene or decalin; chlorobenzene, dichlorobenzene, dichloromethane, chloroform, carbon tetrachloride, dichlorethane or trichlorethane; ethers, such as diethyl ether, diisopropyl ether, methyl t-butyl ether, methyl t-amyl ether, dioxane, tetrahydrofuran, 1,2-dimethoxyethane, 1,2-diethoxyethane or anisole; nitriles, such as acetonitrile, propionitrile, n- or i-butyronitrile or benzonitrile; amides, such as N,N-dimethylformamide, N,N-dimethylacetamide, N-methylformanilide, N-methylpyrrolidone, or hexamethylphosphoric triamide; alcohols such as methanol, ethanol, propanol, iso-propanol; esters, such as methyl acetate or ethyl acetate, sulfoxides, such as dimethyl sulfoxide, or sulfones, such as sulfolane.

When carrying out process P′1 according to the invention, the amine derivative of formula (II-2) can be employed as its salt, such as chlorhydrate or any other convenient salt.

When carrying out process P′1 according to the invention, 1 mole or an excess of the amine derivative of formula (II) and from 1 to 3 moles of the acid binder can be employed per mole of the reagent of formula (III).

It is also possible to employ the reaction components in other ratios. Work-up is carried out by known methods.

According to a further aspect according to the invention, there is provided a second process P′2 for the preparation of a compound of formula (IB-2-1) wherein T represents S, starting from a compound of formula (IB-2-1) wherein T represents O and illustrated according to the following reaction scheme:

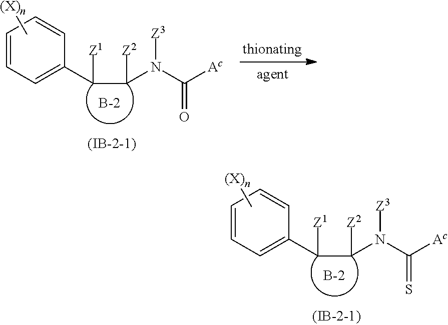

### Process P2

in which Z¹, Z², X and n have the meanings mentioned above for the compounds of formula (IB-2-1),
Z³ represents hydrogen,
B-2 represents a 3- or 4-membered non-aromatic carbocycle optionally substituted by substituents selected from the group consisting of one to four C₁-C₈-alkyl groups and one to four halogen atoms, and
A^{c} represents A²⁶.

Process P′2 according to the invention is performed in the presence of a thionating agent.

Amide derivatives of formula (IB-2-1) wherein T represents O can be prepared according to processes P′1.

Suitable thionating agents for carrying out process P′2 according to the invention can be sulfur (S), sulfhydric acid (H₂S), sodium sulfide (Na₂S), sodium hydrosulfide (NaHS), boron trisulfide (B₂S₃), bis(diethylaluminium) sulfide ((AlEt₂)₂S), ammonium sulfide ((NH₄)₂S), phosphorous pentasulfide (P₂S₅), Lawesson's reagent (2,4-bis(4-methoxyphenyl)-1,2,3,4-dithiadiphosphetane 2,4-disulfide) or a polymer-supported thionating reagent such as described in Journal of the Chemical Society, Perkin 1 (2001), 358, in the optionally presence of a catalytic or stoichiometric or excess amount, quantity of a base such as an inorganic and organic base. Preference is given to using alkali metal carbonates, such as sodium carbonate, potassium carbonate, potassium bicarbonate, sodium bicarbonate; heterocyclic aromatic bases, such as pyridine, picoline, lutidine, collidine; and also tertiary amines, such as trimethylamine, triethylamine, tributylamine, N,N-dimethylaniline, N,N-dimethylpyridin-4-amine or N-methyl-piperidine.

Suitable solvents for carrying out process P′2 according to the invention can be customary inert organic solvents. Preference is given to using optionally halogenated aliphatic, alicyclic or aromatic hydrocarbons, such as petroleum ether, hexane, heptane, cyclohexane, methylcyclohexane, benzene, toluene, xylene or decalin, chlorobenzene, dichlorobenzene, dichloromethane, chloroform, carbon tetrachloride, dichlorethane or trichlorethane, ethers, such as diethyl ether, diisopropyl ether, methyl t-butyl ether, methyl t-amyl ether, dioxane, tetrahydrofuran, 1,2-dimethoxyethane or 1,2-diethoxyethane, nitriles, such as acetonitrile, propionitrile, n- or i-butyronitrile or benzonitrile, sulfurous solvents, such as sulfolane or carbon disulfide.

When carrying out process P′2 according to the invention, 1 mole or an excess of the sulfur equivalent of the thionating agent and from 1 to 3 moles of the base can be employed per mole of the amide reactant.

It is also possible to employ the reaction components in other ratios. Work-up is carried out by known methods.

Processes P′1 and P′2 according to the invention are generally carried out under atmospheric pressure. It is also possible to operate under elevated or reduced pressure.

When carrying out processes P′1 and P′2 according to the invention, the reaction temperatures can be varied within a relatively wide range. In general, these processes are carried out at temperatures from 0° C. to 200° C., preferably from 10° C. to 160° C. A way to control the temperature for the processes according to the invention is to use micro-wave technology.

In general, the reaction mixture is concentrated under reduced pressure. The residue that remains can be freed by known methods, such as chromatography or crystallization, from any impurities that can still be present.

Work-up is carried out by customary methods. Generally, the reaction mixture is treated with water and the organic phase is separated off and, after drying, concentrated under reduced pressure. If appropriate, the remaining residue can, be freed by customary methods, such as chromatography, crystallization or distillation, from any impurities that may still be present.

The compound according to the present invention can be prepared according to the general processes of preparation described above. It will nevertheless be understood that, on the basis of his general knowledge and of available publications, the skilled worker will be able to adapt this method according to the specifics of each of the compounds, which it is desired to synthesize.

Still in a further aspect, the present invention relates to compounds of formula (IIa) useful as intermediate compounds or materials for the process of preparation according to the invention. The present invention thus provides compounds of formula (IIa):

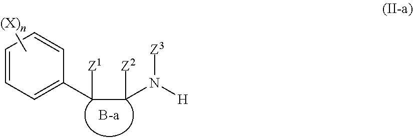

in which Z¹ and Z², have the meanings mentioned above for the compounds of formula (I), n represents 1,2, 3, 4 or 5, and
X represents halogen, nitro, cyano, pentafluoro-λ⁶-sulfanyl, substituted or unsubstituted C₁-C₈-alkyl, C₁-C₈-halogenoalkyl having 1 to 9 halogen atoms, substituted or unsubstituted C₂-C₈-alkenyl, C₂-C₈-halogenoalkenyl having 1 to 9 halogen atoms, substituted or unsubstituted C₂-C₈-alkynyl, C₂-C₈-halogenoalkynyl having 1 to 9 halogen atoms, C₁-C₈-halogenoalkoxy having 1 to 9 halogen atoms, substituted or unsubstituted C₁-C₈-alkylsulfanyl, C₁-C₈-halogenoalkylsulfanyl having 1 to 9 halogen atoms, substituted or unsubstituted C₁-C₈-alkylsulfinyl, C₁-C₈-halogenoalkylsulfinyl having 1 to 9 halogen atoms, substituted or unsubstituted C₁-C₈-alkylsulfonyl, C₁-C₈-halogenoalkylsulfonyl having 1 to 9 halogen atoms, substituted or unsubstituted C₂-C₈-alkenyloxy, C₂-C₈-halogenoalkenyloxy having 1 to 9 halogen atoms, substituted or unsubstituted C₃-C₈-alkynyloxy, C₃-C₈-halogenoalkynyloxy having 1 to 9 halogen atoms, substituted or unsubstituted C₃-C₇-cycloalkyl, C₃-C₇-halogenocycloalkyl having 1 to 9 halogen atoms, substituted or unsubstituted C₄-C₇-cycloalkenyl, C₄-C₇-halogenocycloalkenyl having 1 to 9 halogen atoms, substituted or unsubstituted (C₃-C₇-cycloalkyl)-C₁-C₈-alkyl, substituted or unsubstituted (C₃-C₇-cycloalkyl)-C₂-C₈-alkenyl, substituted or unsubstituted (C₃-C₇-cycloalkyl)-C₂-C₈-alkynyl, substituted or unsubstituted tri-(C₁-C₈-alkyl)-silyl, substituted or unsubstituted tri-(C₁-C₈-alkyl)-silyl-C₁-C₈-alkyl, aryl optionally substituted by 1 to 6 groups Q which can be the same or different, aryl-C₁-C₈-alkyl optionally substituted by 1 to 6 groups Q which can be the same or different, aryl-C₂-C₈-alkenyl optionally substituted by 1 to 6 groups Q which can be the same or different, aryl-C₂-C₈-alkynyl optionally substituted by 1 to 6 groups Q which can be the same or different, aryloxy optionally substituted by 1 to 6 groups Q which can be the same or different, arylsulfanyl optionally substituted by 1 to 6 groups Q which can be the same or different, arylamino optionally substituted by 1 to 6 groups Q which can be the same or different, aryl-C₁-C₈-alkyloxy optionally substituted by 1 to 6 groups Q which can be the same or different, aryl-C₁-C₈-alkylsulfanyl optionally substituted by 1 to 6 groups Q which can be the same or different, pyridinyl which can be substituted by 1 to 4 groups Q, pyridinyloxy which is optionally substituted by 1 to 4 groups Q,
Q has the meaning as described above and,
B-a represents a 4-membered non-aromatic carbocycle optionally substituted by substituents selected from the group consisting of one to four C₁-C₈-alkyl groups and one to four halogen atoms, and
Z³ represents hydrogen, provided that (II-a) does not represent 2-(4-chlorophenyl)-3-fluoro-cyclobutanamine, 2-(2-chlorophenyl)-cyclobutanamine, 2-(2,5-dimethoxy-4-methylphenyl)-, trans-cyclobutanamine.

A “nematicide” as used herein means that the compound is capable of controlling nematodes.

“Controlling nematodes” according to the invention shall mean to kill nematodes or to prevent their development or growth. The efficacy of the compositions or combinations according to the invention is assessed by comparing the mortality of nematodes, the development of galls, the formation of cysts, the concentration of nematodes per volume of soil, of cysts, the concentration of nematodes per root, the number of nematode eggs per volume of soil, the motility of the nematodes between a plant, a plant part or the soil treated with a composition or combination according to the invention and the untreated plant, plant part or soil (100%). Preferred is a reduction by 25-50% in comparison with the untreated plant, plant part or soil, very preferred a reduction by 51-79%, and particularly preferred the complete killing and the complete prevention of the development or growth by a reduction from 80% to 100% in comparison with the untreated plant, plant part or soil.

“Controlling nematodes” according to the invention shall mean the control of the reproduction of the nematodes (e.g. development of cysts or eggs). The compositions according to the invention can be used for keeping the plants healthy and can be used curatively, preventively or systemically for controlling nematodes.

The skilled person knows methods for determining the mortality of nematodes, the development of galls, the formation of cysts, the concentration of nematodes per volume of soil, of cysts, the concentration of nematodes per root, the number of nematode eggs per volume of soil, the motility of the nematodes between a plant, a plant part or the soil. The treatment according to the invention reduces the damages caused by nematodes to the plant and leads to an increase in yield.

“Nematodes” as used herein encompass all species of the phylum Nematoda and in particular species that are parasitic or cause health problems to plant or to fungi (for example species of the orders Aphelenchida, Meloidogyne, Tylenchida and others) or to humans and animals (for example species of the orders Trichinellida, Tylenchida, Rhabditina, and Spirurida) as well as other parasitic helminths.

“Nematodes” as used herein, refer to plant nematodes meaning all nematodes that cause damage to plants. Plant nematodes encompass plant parasitic nematodes and nematodes living in the soil. Plant parasitic nematodes include, but are not limited to, ectoparasites such as Xiphinema spp., Longidorus spp., and Trichodorus spp.; semiparasites such as Tylenchulus spp.; migratory endoparasites such as Pratylenchus spp., Radopholus spp., and Scutellonerna spp.; sedentary parasites such as Heterodera spp., Globodera spp., and Meloidogyne spp., and stem and leaf endoparasites such as Ditylenchus spp., Aphelenchoides spp., and Hirshmaniella spp. Especially harmful root parasitic soil nematodes are such as cystforming nematodes of the genera Heterodera or Globodera, and/or root knot nematodes of the genus Meloidogyne. Harmful species of these genera are for example Meloidogyne incognita, Heterodera glycines (soybean cyst nematode), Globodera pallida and Globodera rostochiensis (potato cyst nematode), which species are effectively controlled with the compounds described herein. However, the use of the compounds described herein is in no way restricted to these genera or species, but also extends in the same manner to other nematodes.

Plant nematodes include but are not limited to e.g. Aglenchus agricola, Anguina tritici, Aphelenchoides arachidis, Aphelenchoides fragaria and the stem and leaf endoparasites Aphelenchoides spp. in general, Belonolaimus gracilis, Belonolaimus longicaudatus, Belonolaimus nortoni, Bursaphelenchus cocophilus, Bursaphelenchus eremus, Bursaphelenchus xylophilus and Bursaphelenchus spp. in general, Cacopaurus pestis, Criconemella curvata, Criconemella onoensis, Criconemella ornata, Criconemella rusium, Criconemella xenoplax (=Mesocriconema xenoplax) and Criconemella spp. in general, Criconemoides ferniae, Criconemoides onoense, Criconemoides ornatum and Criconemoides spp. in general, Ditylenchus destructor, Ditylenchus dipsaci, Ditylenchus myceliophagus and the stem and leaf endoparasites Ditylenchus spp. in general, Dolichodorus heterocephalus, Globodera pallida (=Heterodera pallida), Globodera rostochiensis (potato cyst nematode), Globodera solanacearum, Globodera tabacum, Globodera virginia and the sedentary, cyst forming parasites Globodera spp. in general, Helicotylenchus digonicus, Helicotylenchus dihystera, Helicotylenchus erythrine, Helicotylenchus multicinctus, Helicotylenchus nannus, Helicotylenchus pseudorobustus and Helicotylenchus spp. in general, Hemicriconemoides, Hemicycliophora arenaria, Hemicycliophora nudata, Hemicycliophora parvana, Heterodera avenae, Heterodera cruciferae, Heterodera glycines (soybean cyst nematode), Heterodera oryzae, Heterodera schachtii, Heterodera zeae and the sedentary, cyst forming parasites Heterodera spp. in general, Hirschmaniella gracilis, Hirschmaniella oryzae Hirschmaniella spinicaudata and the stem and leaf endoparasites Hirschmaniella spp. in general, Hoplolaimus aegyptii, Hoplolaimus californicus, Hoplolaimus columbus, Hoplolaimus galeatus, Hoplolaimus indicus, Hoplolaimus magnistylus, Hoplolaimus pararobustus, Longidorus africanus, Longidorus breviannulatus, Longidorus elongatus, Longidorus laevicapitatus, Longidorus vineacola and the ectoparasites Longidorus spp. in general, Meloidogyne acronea, Meloidogyne africana, Meloidogyne arenaria, Meloidogyne arenaria thamesi, Meloidogyne artiella, Meloidogyne chitwoodi, Meloidogyne coffeicola, Meloidogyne ethiopica, Meloidogyne exigua, Meloidogyne fallax, Meloidogyne graminicola, Meloidogyne graminis, Meloidogyne hapla, Meloidogyne incognita, Meloidogyne incognita acrita, Meloidogyne javanica, Meloidogyne kikuyensis, Meloidogyne minor, Meloidogyne naasi, Meloidogyne paranaensis, Meloidogyne thamesi and the sedentary parasites Meloidogyne spp. in general, Meloinema spp., Nacobbus aberrans, Neotylenchus vigissi, Paraphelenchus pseudoparietinus, Paratrichodorus allius, Paratrichodorus lobatus, Paratrichodorus minor, Paratrichodorus nanus, Paratrichodorus porosus, Paratrichodorus teres and Paratrichodorus spp. in general, Paratylenchus hamatus, Paratylenchus minutus, Paratylenchus projectus and Paratylenchus spp. in general, Pratylenchus agilis, Pratylenchus alleni, Pratylenchus andinus, Pratylenchus brachyurus, Pratylenchus cerealis, Pratylenchus coffeae, Pratylenchus crenatus, Pratylenchus as delattrei, Pratylenchus giibbicaudatus, Pratylenchus goodeyi, Pratylenchus hamatus, Pratylenchus hexincisus, Pratylenchus loosi, Pratylenchus neglectus, Pratylenchus penetrans, Pratylenchus pratensis, Pratylenchus scribneri, Pratylenchus teres, Pratylenchus thornei, Pratylenchus vulnus, Pratylenchus zeae and the migratory endoparasites Pratylenchus spp. in general, Pseudohalenchus minutus, Psilenchus magnidens, Psilenchus tumidus, Punctodera chalcoensis, Quinisulcius acutus, Radopholus citrophilus, Radopholus similis, the migratory endoparasites Radopholus spp. in general, Rotylenchulus borealis, Rotylenchulus parvus, Rotylenchulus reniformis and Rotylenchulus spp. in general, Rotylenchus laurentinus, Rotylenchus macrodoratus, Rotylenchus robustus, Rotylenchus uniformis and Rotylenchus spp. in general, Scutellonema brachyurum, Scutellonema bradys, Scutellonema clathricaudatum and the migratory endoparasites Scutellonema spp. in general, Subanguina radiciola, Tetylenchus nicotianae, Trichodorus cylindricus, Trichodorus minor, Trichodorus primitivus, Trichodorus proximus, Trichodorus similis, Trichodorus sparsus and the ectoparasites Trichodorus spp. in general, Tylenchorhynchus agri, Tylenchorhynchus brassicae, Tylenchorhynchus clarus, Tylenchorhynchus claytoni, Tylenchorhynchus digitatus, Tylenchorhynchus ebriensis, Tylenchorhynchus maximus, Tylenchorhynchus nudus, Tylenchorhynchus vulgaris and Tylenchorhynchus spp. in general, Tylenchulus semipenetrans and the semiparasites Tylenchulus spp. in general, Xiphinema americanum, Xiphinema brevicolle, Xiphinema dimorphicaudatum, Xiphinema index and the ectoparasites Xiphinema spp. in general.

Examples of nematodes to which a nematicide of the present invention is applicable include, but are not limited to, nematodes of the genus Meloidogyne such as the southern root-knot nematode (Meloidogyne incognita), Javanese root-knot nematode (Meloidogyne javanica), northern root-knot nematode (Meloidogyne hapla), and peanut root-knot nematode (Meloidogyne arenaria); nematodes of the genus Ditylenchus such as the potato rot nematode (Ditylenchus destructor) and bulb and stem nematode (Ditylenchus dipsaci); nematodes of the genus Pratylenchus such as the cob root-lesion nematode (Pratylenchus penetrans), chrysanthemum root-lesion nematode (Pratylenchus fallax), coffee root-lesion nematode (Pratylenchus coffeae), tea root-lesion nematode (Pratylenchus loosi), and walnut root-lesion nematode (Pratylenchus vulnus); nematodes of the genus Globodera such as the golden nematode (Globodera rostochiensis) and potato cyst nematode (Globodera pallida); nematodes of the genus Heterodera such as the soybean cyst nematode (Heterodera glycines) and sugar beet cyst nematode (Heterodera schachtii); nematodes of the genus Aphelenchoides such as the rice white-tip nematode (Aphelenchoides besseyi), chrysanthemum foliar nematode (Aphelenchoides ritzemabosi), and strawberry nematode (Aphelenchoides fragariae); nematodes of the genus Aphelenchus such as the mycophagous nematode (Aphelenchus avenae); nematodes of the genus Radopholus such as the burrowing nematode (Radopholus similis); nematodes of the genus Tylenchulus such as the citrus nematode (Tylenchulus semipenetrans); nematodes of the genus Rotylenchulus such as the reniform nematode (Rotylenchulus reniformis); nematodes that occur in trees, such as the pine wood nematode (Bursaphelenchus xylophilus), and the like.

Plants for which a nematicide of the present invention can be used are not particularly limited; for example, plants such as cereals (for example, rice, barley, wheat, rye, oat, corn, and the like), beans (soybeans, azuki beans, broad beans, peas, peanuts and the like), fruit trees/fruits (apples, citrus species, pears, grapes, peaches, Japanese apricots, cherries, walnuts, almonds, bananas, strawberries and the like), vegetables (cabbage, tomato, spinach, broccoli, lettuce, onion, Welsh onion, pepper and the like), root crops (carrot, potato, sweet potato, radish, lotus root, turnip and the like), industrial crops (cotton, hemp, paper mulberry, mitsumata, rape, beet, hop, sugarcane, sugar beet, olive, rubber, palms, coffee, tobacco, tea and the like), pepos (pumpkin, cucumber, watermelon, melon and the like), pasture plants (orchard grass, sorghum, thimosy, clover, alfalfa and the like), lawn grasses (mascarene grass, bent grass and the like), crops for flavorings etc. (lavender, rosemary, thyme, parsley, pepper, ginger and the like), and flower plants (chrysanthemum, rose, orchids and the like) can be mentioned.

The compound(s) and compositions comprising the compound(s) of the present invention is/are particularly useful in controlling nematodes in coffee belonging to at least one species selected from the group of the phytoparasitic nematodes consisting of Pratylenchus brachyurus, Pratylenchus coffeae, Meloidogyne exigua, Meloidogyne incognita, Meloidogyne coffeicola, Helicotylenchus spp. and also consisting of Meloidogyne paranaensis, Rotylenchus spp., Xiphinema spp., Tylenchorhynchus spp., Scutellonema spp.

Compound(s) and compositions comprising compound(s) of the present invention is/are particularly useful in controlling nematodes in potato belonging to at least one species selected from the group of the phytoparasitic nematodes consisting of Pratylenchus brachyurus, Pratylenchus pratensis, Pratylenchus scribneri, Pratylenchus penetrans, Pratylenchus coffeae, Ditylenchus dipsaci and also consisting of Pratylenchus alleni, Pratylenchus andinus, Pratylenchus cerealis, Pratylenchus crenatus, Pratylenchus hexincisus, Pratylenchus loosi, Pratylenchus neglectus, Pratylenchus teres, Pratylenchus thornei, Pratylenchus vulnus, Belonolaimus longicaudatus, Trichodorus cylindricus, Trichodorus primitivus, Trichodorus proximus, Trichodorus similis, Trichodorus sparsus, Paratrichodorus minor, Paratrichodorus allius, Paratrichodorus nanus, Paratrichodorus teres, Meloidogyne arenaria, Meloidogyne fallax, Meloidogyne hapla, Meloidogyne thamesi, Meloidogyne incognita, Meloidogyne chitwoodi, Meloidogyne javanica, Nacobbus aberrans, Globodera rostochiensis, Globodera pallida, Ditylenchus destructor, Radopholus similis, Rotylenchulus reniformis, Neotylenchus vigissi, Paraphelenchus pseudoparietinus, Aphelenchoides fragariae, Meloinema spp.

Compound(s) and compositions comprising the compound(s) of the present invention is/are particularly useful in controlling nematodes in tomato belonging to at least one species selected from the group of the phytoparasitic nematodes consisting of Meloidogyne arenaria, Meloidogyne hapla, Meloidogyne javanica, Meloidogyne incognita, Pratylenchus penetrans and also consisting of Pratylenchus brachyurus, Pratylenchus coffeae, Pratylenchus scribneri, Pratylenchus vulnus, Paratrichodorus minor, Meloidogyne exigua, Nacobbus aberrans, Globodera solanacearum, Dolichodorus heterocephalus, Rotylenchulus reniformis.

The compound(s) and compositions comprising the compound(s) of the present invention is/are particularly useful in controlling nematodes in cucurbits belonging to at least one species selected from the group of the phytoparasitic nematodes consisting of Meloidogyne arenaria, Meloidogyne hapla, Meloidogyne javanica, Meloidogyne incognita, Rotylenchulus reniformis and also consisting of Pratylenchus thornei.

The compound(s) and compositions comprising the compound(s) of the present invention is/are particularly useful in controlling nematodes in cotton belonging to at least one species selected from the group of the phytoparasitic nematodes consisting of Belonolaimus longicaudatus, Meloidogyne incognita, Hoplolaimus columbus, Hoplolaimus galeatus, Rotylenchulus reniformis.

The compound(s) and compositions comprising the compound(s) of the present invention is/are particularly useful in controlling nematodes in corn belonging to at least one species selected from the group of the phytoparasitic nematodes, especially consisting of Belonolaimus longicaudatus, Paratrichodorus minor and also consisting of Pratylenchus brachyurus, Pratylenchus delattrei, Pratylenchus hexincisus, Pratylenchus penetrans, Pratylenchus zeae, (Belonolaimus gracilis), Belonolaimus nortoni, Longidorus breviannulatus, Meloidogyne arenaria, Meloidogyne arenaria thamesi, Meloidogyne graminis, Meloidogyne incognita, Meloidogyne incognita acrita, Meloidogyne javanica, Meloidogyne naasi, Heterodera avenae, Heterodera oryzae, Heterodera zeae, Punctodera chalcoensis, Ditylenchus dipsaci, Hoplolaimus aegyptii, Hoplolaimus magnistylus, Hoplolaimus galeatus, Hoplolaimus indicus, Helicotylenchus digonicus, Helicotylenchus dihystera, Helicotylenchus pseudorobustus, Xiphinema americanum, Dolichodorus heterocephalus, Criconemella ornata, Criconemella onoensis, Radopholus similis, Rotylenchulus borealis, Rotylenchulus parvus, Tylenchorhynchus agri, Tylenchorhynchus clarus, Tylenchorhynchus claytoni, Tylenchorhynchus maximus, Tylenchorhynchus nudus, Tylenchorhynchus vulgaris, Quinisulcius acutus, Paratylenchus minutus, Hemicycliophora parvana, Aglenchus agricola, Anguina tritici, Aphelenchoides arachidis, Scutellonema brachyurum, Subanguina radiciola.

The compound(s) and compositions comprising the compound(s) of the present invention is/are particularly useful in controlling nematodes in soybean belonging to at least one species selected from the group of the phytoparasitic nematodes, especially consisting of Pratylenchus brachyurus, Pratylenchus pratensis, Pratylenchus penetrans, Pratylenchus scribneri, Belonolaimus longicaudatus, Heterodera glycines, Hoplolaimus columbus and also consisting of Pratylenchus coffeae, Pratylenchus hexincisus, Pratylenchus neglectus, Pratylenchus crenatus, Pratylenchus alleni, Pratylenchus agilis, Pratylenchus zeae, Pratylenchus vulnus, (Belonolaimus gracilis), Meloidogyne arenaria, Meloidogyne incognita, Meloidogyne javanica, Meloidogyne hapla, Hoplolaimus columbus, Hoplolaimus galeatus, Rotylenchulus reniformis.

The compound(s) and compositions comprising the compound(s) of the present invention is/are particularly useful in controlling nematodes in tobacco belonging to at least one species selected from the group of the phytoparasitic nematodes, especially consisting of Meloidogyne incognita, Meloidogyne javanica and also consisting of Pratylenchus brachyurus, Pratylenchus pratensis, Pratylenchus hexincisus, Pratylenchus penetrans, Pratylenchus neglectus, Pratylenchus crenatus, Pratylenchus thornei, Pratylenchus vulnus, Pratylenchus zeae, Longidorus elongatu, Paratrichodorus lobatus, Trichodorus spp., Meloidogyne arenaria, Meloidogyne hapla, Globodera tabacum, Globodera solanacearum, Globodera virginiae, Ditylenchus dipsaci, Rotylenchus spp., Helicotylenchus spp., Xiphinema americanum, Criconemella spp., Rotylenchulus reniformis, Tylenchorhynchus claytoni, Paratylenchus spp., Tetylenchus nicotianae.

The compound(s) and compositions comprising the compound(s) of the present invention is/are particularly useful in controlling nematodes in citrus belonging to at least one species selected from the group of the phytoparasitic nematodes, especially consisting of Pratylenchus coffeae and also consisting of Pratylenchus brachyurus, Pratylenchus vulnus, Belonolaimus longicaudatus, Paratrichodorus minor, Paratrichodorus porosus, Trichodorus, Meloidogyne incognita, Meloidogyne incognita acrita, Meloidogyne javanica, Rotylenchus macrodoratus, Xiphinema americanum, Xiphinema brevicolle, Xiphinema index, Criconemella spp., Hemicriconemoides, Radopholus similis respectively Radopholus citrophilus, Hemicycliophora arenaria, Hemicycliophora nudata, Tylenchulus semipenetrans.

The compound(s) and compositions comprising the compound(s) of the present invention is/are particularly useful in controlling nematodes in banana belonging to at least one species selected from the group of the phytoparasitic nematodes, especially consisting of Pratylenchus coffeae, Radopholus similis and also consisting of Pratylenchus giibbicaudatus, Pratylenchus loosi, Meloidogyne spp., Helicotylenchus multicinctus, Helicotylenchus dihystera, Rotylenchulus spp.

The compound(s) and compositions comprising the compound(s) of the present invention is/are particularly useful in controlling nematodes in pine apple belonging to at least one species selected from the group of the phytoparasitic nematodes, especially consisting of Pratylenchus zeae, Pratylenchus pratensis, Pratylenchus brachyurus, Pratylenchus goodeyi, Meloidogyne spp., Rotylenchulus reniformis and also consisting of Longidorus elongatus, Longidorus laevicapitatus, Trichodorus primitivus, Trichodorus minor, Heterodera spp., Ditylenchus myceliophagus, Hoplolaimus californicus, Hoplolaimus pararobustus, Hoplolaimus indicus, Helicotylenchus dihystera, Helicotylenchus nannus, Helicotylenchus multicinctus, Helicotylenchus erythrine, Xiphinema dimorphicaudatum, Radopholus similis, Tylenchorhynchus digitatus, Tylenchorhynchus ebriensis, Paratylenchus minutus, Scutellonema clathricaudatum, Scutellonema bradys, Psilenchus tumidus, Psilenchus magnidens, Pseudohalenchus minutus, Criconemoides ferniae, Criconemoides onoense, Criconemoides ornatum.

The compound(s) and compositions comprising the compound(s) of the present invention is/are particularly useful in controlling nematodes in grapes belonging to at least one species selected from the group of the phytoparasitic nematodes, especially consisting of Pratylenchus vulnus, Meloidogyne arenaria, Meloidogyne incognita, Meloidogyne javanica, Xiphinema americanum, Xiphinema index and also consisting of Pratylenchus pratensis, Pratylenchus scribneri, Pratylenchus neglectus, Pratylenchus brachyurus, Pratylenchus thornei, Tylenchulus semipenetrans.

The compound(s) and compositions comprising the compound(s) of the present invention is/are particularly useful in controlling nematodes in tree crops—pome fruits, belonging to at least one species selected from the group of the phytoparasitic nematodes, especially consisting of Pratylenchus penetrans and also consisting of Pratylenchus vulnus, Longidorus elongatus, Meloidogyne incognita, Meloidogyne hapla.

The compound(s) and compositions comprising the compound(s) of the present invention is/are particularly useful in controlling nematodes in tree crops—stone fruits, belonging to at least one species selected from the group of the phytoparasitic nematodes, especially consisting of Pratylenchus penetrans, Pratylenchus vulnus, Meloidogyne arenaria, Meloidogyne hapla, Meloidogyne javanica, Meloidogyne incognita, Criconemella xenoplax and also consisting of Pratylenchus brachyurus, Pratylenchus coffeae, Pratylenchus scribneri, Pratylenchus zeae, Belonolaimus longicaudatus, Helicotylenchus dihystera, Xiphinema americanum, Criconemella curvata, Tylenchorhynchus claytoni, Paratylenchus hamatus, Paratylenchus projectus, Scutellonema brachyurum, Hoplolaimus galeatus.

The compound(s) and compositions comprising the compound(s) of the present invention is/are particularly useful in controlling nematodes in tree crops—nuts, belonging to at least one species selected from the group of the phytoparasitic nematodes, especially consisting of Trichodorus spp., Criconemella rusium and also consisting of Pratylenchus vulnus, Paratrichodorus spp., Meloidogyne incognita, Helicotylenchus spp., Tylenchorhynchus spp., Cacopaurus pestis.

In a like manner, “nematodes” as used herein, refer to nematodes which cause damage to humans or animals.

Specific nematode species harmful to humans or animals are:

Trichinellida for example: Trichuris spp., Capillaria spp., Trichomosoides spp., Trichinella spp.

From the order of the Tylenchida for example: Micronema spp., Strongyloides spp.

From the order of the Rhabditina for example: Strongylus spp., Triodontophorus spp., Oesophagodontus spp., Trichonema spp., Gyalocephalus spp., Cylindropharynx spp., Poteriostomum spp., Cyclococercus spp., Cylicostephanus spp., Oesophagostomum spp., Chabertia spp., Stephanurus spp., Ancylostoma spp., Uncinaria spp., Bunostomum spp., Globocephalus spp., Syngamus spp., Cyathostoma spp., Metastrongylus spp., Dictyocaulus spp., Muellerius spp., Protostrongylus spp., Neostrongylus spp., Cystocaulus spp., Pneumostrongylus spp., Spicocaulus spp., Elaphostrongylus spp. Parelaphostrongylus spp., Crenosoma spp., Paracrenosoma spp., Angiostrongylus spp., Aelurostrongylus spp., Filaroides spp., Parafilaroides spp., Trichostrongylus spp., Haemonchus spp., Ostertagia spp., Marshallagia spp., Cooperia spp., Nematodirus spp., Hyostrongylus spp., Obeliscoides spp., Amidostomum spp., Ollulanus spp.

From the order of the Spirurida for example: Oxyuris spp., Enterobius spp., Passalurus spp., Syphacia spp., Aspiculuris spp., Heterakis spp.; Ascaris spp., Toxascaris spp., Toxocara spp., Baylisascaris spp., Parascaris spp., Anisakis spp., Ascaridia spp.; Gnathostoma spp., Physaloptera spp., Thelazia spp., Gongylonema spp., Habronema spp., Parabronema spp., Draschia spp., Dracunculus spp.; Stephanofilaria spp., Parafilaria spp., Setaria spp., Loa spp., Dirofilaria spp., Litomosoides spp., Brugia spp., Wuchereria spp., Onchocerca spp.

Many known nematicides are equally active against other parasitic helminths and are therefore used to control human and animal parasitic worms, which do not necessarily belong to the group of nematoda. Therefore, it is envisaged by the present invention that the compounds described herein may also be used as anthelmintic drugs in a more general meaning. Pathogenic endoparasitic helminths include platyhelmintha (e.g. monogenea, cestodes and trematodes), acanthocephala, and pentastoma. The following helminths may be mentioned by way of example and by way of preference—but without any limitation:

Monogenea: e.g.: Gyrodactylus spp., Dactylogyrus spp., Polystoma spp.

Cestodes: From the order of the Pseudophyllidea for example: Diphyllobothrium spp., Spirometra spp., Schistocephalus spp., Ligula spp., Bothridium spp., Diplogonoporus spp.

From the order of the Cyclophyllida for example: Mesocestoides spp., Anoplocephala spp., Paranoplocephala spp., Moniezia spp., Thysanosoma spp., Thysaniezia spp., Avitellina spp., Stilesia spp., Cittotaenia spp., Andyra spp., Bertiella spp., Taenia spp., Echinococcus spp., Hydatigera spp., Davainea spp., Raillietina spp., Hymenolepis spp., Echinolepis spp., Echinocotyle spp., Diorchis spp., Dipylidium spp., Joyeuxiella spp., Diplopylidium spp.

Trematodes: From the class of the Digenea for example: Diplostomum spp., Posthodiplostomum spp., Schistosoma spp., Trichobilharzia spp., Ornithobilharzia spp., Austrobilharzia spp., Gigantobilharzia spp., Leucochloridium spp., Brachylaima spp., Echinostoma spp., Echinoparyphium spp., Echinochasmus spp., Hypoderaeum spp., Fasciola spp., Fasciolides spp., Fasciolopsis spp., Cyclocoelum spp., Typhlocoelum spp., Paramphistomum spp., Calicophoron spp., Cotylophoron spp., Gigantocotyle spp., Fischoederius spp., Gastrothylacus spp., Notocotylus spp., Catatropis spp., Plagiorchis spp., Prosthogonimus spp., Dicrocoelium spp., Eurytrema spp., Troglotrema spp., Paragonimus spp., Collyriclum spp., Nanophyetus spp., Opisthorchis spp., Clonorchis spp., Metorchis spp., Heterophyes spp., Metagonimus spp.

Acantocephala: From the order of the Oligacanthorhynchida z.B: Macracanthorhynchus spp., Prosthenorchis spp.; from the order of the Polymorphida for example: Filicollis spp.; from the order of the Moniliformida for example: Moniliformis spp.,

From the order of the Echinorhynchida for example Acanthocephalus spp., Echinorhynchus spp., Leptorhynchoides spp.

Pentastoma: From the order of the Porocephalida for example Linguatula spp.

In the veterinary field and in animal keeping, the administration of the active compounds according to the invention is carried out in the known manner directly or enterally, parenterally, dermally or nasally in the form of suitable preparations. Administration can be carried out prophylactically or therapeutically.

Thus, one embodiment of the present invention refers to compounds according to the invention for use as a medicament.

Another aspect refers to compounds according to the invention for use as an antiendoparasitical agent, in particular a helmithicidal agent or antiprotozoaic agent. For example, compounds according to the invention for use as an antiendoparasitical agent, in particular an helmithicidal agent or antiprotozoaic agent, e.g., in animal husbandry, in animal breeding, in animal housing, in the hygiene sector.

Yet another aspect refers to compounds according to the invention for use as an antiectoparasitical agent, in particular an arthropodicidal agent such as an insecticidal agent or acaricidal agent. For example, compounds according to the invention for use as an antiectoparasitical agent, in particular an arthropodicidal agent such as an insecticidal agent or acaricidal agent, e.g., in animal husbandry, in animal breeding, in animal housing, in the hygiene sector.

A further aspect of the invention are nematicidal compositions, comprising an effective amount of at least one compound as defined herein and at least one of the following: surfactant, solid or liquid diluent, characterized in that the surfactant or the diluent is normally used in nematicidal compositions. In an embodiment, said composition comprises at least two compounds as defined herein.

A related aspect of the invention is a method for preparing a nematicidal composition as described herein, comprising the step of mixing at least one compound as described herein with a surfactant or diluent normally used in nematicidal compositions. In an embodiment, said method comprises mixing least two compounds as defined herein with a surfactant or diluent normally used in nematicidal compositions.

In particular, the present invention relates to nematicidal composition developed to be used in agriculture or horticulture. These nematicidal compositions may be prepared in a manner known per se.

The present invention further provides formulations, and application forms prepared from them, as crop protection agents and/or pesticidal agents, such as drench, drip and spray liquors, comprising at least one of the active compounds of the invention. The application forms may comprise further crop protection agents and/or pesticidal agents, and/or activity-enhancing adjuvants such as penetrants, examples being vegetable oils such as, for example, rapeseed oil, sunflower oil, mineral oils such as, for example, liquid paraffins, alkyl esters of vegetable fatty acids, such as rapeseed oil or soybean oil methyl esters, or alkanol alkoxylates, and/or spreaders such as, for example, alkylsiloxanes and/or salts, examples being organic or inorganic ammonium or phosphonium salts, examples being ammonium sulphate or diammonium hydrogen phosphate, and/or retention promoters such as dioctyl sulphosuccinate or hydroxypropylguar polymers and/or humectants such as glycerol and/or fertilizers such as ammonium, potassium or phosphorous fertilizers, for example.

Examples of typical formulations include water-soluble liquids (SL), emulsifiable concentrates (EC), emulsions in water (EW), suspension concentrates (SC, SE, FS, OD), water-dispersible granules (WG), granules (GR) and capsule concentrates (CS); these and other possible types of formulation are described, for example, by Crop Life International and in Pesticide Specifications, Manual on development and use of FAO and WHO specifications for pesticides, FAO Plant Production and Protection Papers—173, prepared by the FAO/WHO Joint Meeting on Pesticide Specifications, 2004, ISBN: 9251048576. The formulations may comprise active agrochemical compounds other than one or more active compounds of the invention.

The formulations or application forms in question preferably comprise auxiliaries, such as extenders, solvents, spontaneity promoters, carriers, emulsifiers, dispersants, frost protectants, biocides, thickeners and/or other auxiliaries, such as adjuvants, for example. An adjuvant in this context is a component which enhances the biological effect of the formulation, without the component itself having a biological effect. Examples of adjuvants are agents which promote the retention, spreading, attachment to the leaf surface, or penetration.

These formulations are produced in a known manner, for example by mixing the active compounds with auxiliaries such as, for example, extenders, solvents and/or solid carriers and/or further auxiliaries, such as, for example, surfactants. The formulations are prepared in suitable plants or else before or during the application.

Suitable for use as auxiliaries are substances which are suitable for imparting to the formulation of the active compound or the application forms prepared from these formulations (such as, e.g., usable crop protection agents, such as spray liquors or seed dressings) particular properties such as certain physical, technical and/or biological properties.

Suitable extenders are, for example, water, polar and nonpolar organic chemical liquids, for example from the classes of the aromatic and non-aromatic hydrocarbons (such as paraffins, alkylbenzenes, alkylnaphthalenes, chlorobenzenes), the alcohols and polyols (which, if appropriate, may also be substituted, etherified and/or esterified), the ketones (such as acetone, cyclohexanone), esters (including fats and oils) and (poly)ethers, the unsubstituted and substituted amines, amides, lactams (such as N-alkylpyrrolidones) and lactones, the sulphones and sulphoxides (such as dimethyl sulphoxide).

If the extender used is water, it is also possible to employ, for example, organic solvents as auxiliary solvents. Essentially, suitable liquid solvents are: aromatics such as xylene, toluene or alkylnaphthalenes, chlorinated aromatics and chlorinated aliphatic hydrocarbons such as chlorobenzenes, chloroethylenes or methylene chloride, aliphatic hydrocarbons such as cyclohexane or paraffins, for example petroleum fractions, mineral and vegetable oils, alcohols such as butanol or glycol and also their ethers and esters, ketones such as acetone, methyl ethyl ketone, methyl isobutyl ketone or cyclohexanone, strongly polar solvents such as dimethylformamide and dimethyl sulphoxide, and also water.

In principle it is possible to use all suitable solvents. Suitable solvents are, for example, aromatic hydrocarbons, such as xylene, toluene or alkylnaphthalenes, for example, chlorinated aromatic or aliphatic hydrocarbons, such as chlorobenzene, chloroethylene or methylene chloride, for example, aliphatic hydrocarbons, such as cyclohexane, for example, paraffins, petroleum fractions, mineral and vegetable oils, alcohols, such as methanol, ethanol, isopropanol, butanol or glycol, for example, and also their ethers and esters, ketones such as acetone, methyl ethyl ketone, methyl isobutyl ketone or cyclohexanone, for example, strongly polar solvents, such as dimethyl sulphoxide, and water.

All suitable carriers may in principle be used. Suitable carriers are in particular: for example, ammonium salts and ground natural minerals such as kaolins, clays, talc, chalk, quartz, attapulgite, montmorillonite or diatomaceous earth, and ground synthetic minerals, such as finely divided silica, alumina and natural or synthetic silicates, resins, waxes and/or solid fertilizers. Mixtures of such carriers may likewise be used. Carriers suitable for granules include the following: for example, crushed and fractionated natural minerals such as calcite, marble, pumice, sepiolite, dolomite, and also synthetic granules of inorganic and organic meals, and also granules of organic material such as sawdust, paper, coconut shells, maize cobs and tobacco stalks.

Liquefied gaseous extenders or solvents may also be used. Particularly suitable are those extenders or carriers which at standard temperature and under standard pressure are gaseous, examples being aerosol propellants, such as halogenated hydrocarbons, and also butane, propane, nitrogen and carbon dioxide.

Examples of emulsifiers and/or foam-formers, dispersants or wetting agents having ionic or nonionic properties, or mixtures of these surface-active substances, are salts of polyacrylic acid, salts of lignosulphonic acid, salts of phenolsulphonic acid or naphthalenesulphonic acid, polycondensates of ethylene oxide with fatty alcohols or with fatty acids or with fatty amines, with substituted phenols (preferably alkylphenols or arylphenols), salts of sulphosuccinic esters, taurine derivatives (preferably alkyltaurates), phosphoric esters of polyethoxylated alcohols or phenols, fatty acid esters of polyols, and derivatives of the compounds containing sulphates, sulphonates and phosphates, examples being alkylaryl polyglycol ethers, alkylsulphonates, alkyl sulphates, arylsulphonates, protein hydrolysates, lignin-sulphite waste liquors and methylcellulose. The presence of a surface-active substance is advantageous if one of the active compounds and/or one of the inert carriers is not soluble in water and if application takes place in water.

Further auxiliaries that may be present in the formulations and in the application forms derived from them include colorants such as inorganic pigments, examples being iron oxide, titanium oxide, Prussian Blue, and organic dyes, such as alizarin dyes, azo dyes and metal phthalocyanine dyes, and nutrients and trace nutrients, such as salts of iron, manganese, boron, copper, cobalt, molybdenum and zinc.

Stabilizers, such as low-temperature stabilizers, preservatives, antioxidants, light stabilizers or other agents which improve chemical and/or physical stability may also be present. Additionally present may be foam-formers or defoamers.

Furthermore, the formulations and application forms derived from them may also comprise, as additional auxiliaries, stickers such as carboxymethylcellulose, natural and synthetic polymers in powder, granule or latex form, such as gum arabic, polyvinyl alcohol, polyvinyl acetate, and also natural phospholipids, such as cephalins and lecithins, and synthetic phospholipids. Further possible auxiliaries include mineral and vegetable oils.

There may possibly be further auxiliaries present in the formulations and the application forms derived from them. Examples of such additives include fragrances, protective colloids, binders, adhesives, thickeners, thixotropic substances, penetrants, retention promoters, stabilizers, sequestrants, complexing agents, humectants and spreaders. Generally speaking, the active compounds may be combined with any solid or liquid additive commonly used for formulation purposes.

Suitable retention promoters include all those substances which reduce the dynamic surface tension, such as dioctyl sulphosuccinate, or increase the viscoelasticity, such as hydroxypropylguar polymers, for example.

Suitable penetrants in the present context include all those substances which are typically used in order to enhance the penetration of active agrochemical compounds into plants. Penetrants in this context are defined in that, from the (generally aqueous) application liquor and/or from the spray coating, they are able to penetrate the cuticle of the plant and thereby increase the mobility of the active compounds in the cuticle. This property can be determined using the method described in the literature (Baur et al., 1997, Pesticide Science 51, 131-152). Examples include alcohol alkoxylates such as coconut fatty ethoxylate (10) or isotridecyl ethoxylate (12), fatty acid esters such as rapeseed or soybean oil methyl esters, fatty amine alkoxylates such as tallowamine ethoxylate (15), or ammonium and/or phosphonium salts such as ammonium sulphate or diammonium hydrogen phosphate, for example.

The formulations preferably comprise between 0.00000001% and 98% by weight of active compound or, with particular preference, between 0.01% and 95% by weight of active compound, more preferably between 0.5% and 90% by weight of active compound, based on the weight of the formulation.

The active compound content of the application forms (crop protection products) prepared from the formulations may vary within wide ranges. The active compound concentration of the application forms may be situated typically between 0.00000001% and 95% by weight of active compound, preferably between 0.00001% and 1% by weight, based on the weight of the application form. Application takes place in a customary manner adapted to the application forms.

The compounds are applied in a customary manner appropriate for the use forms.

Preferred plants are those from the group of the useful plants, ornamentals, turfs, generally used trees which are employed as ornamentals in the public and domestic sectors, and forestry trees. Forestry trees comprise trees for the production of timber, cellulose, paper and products made from parts of the trees.

The term useful plants as used in the present context refers to crop plants which are employed as plants for obtaining foodstuffs, feedstuffs, fuels or for industrial purposes.

The useful plants which can be improved by applying the compounds of formula (I) include for example the following types of plants: turf, vines, cereals, for example wheat, barley, rye, oats, rice, maize and millet/sorghum; beet, for example sugar beet and fodder beet; fruits, for example pome fruit, stone fruit and soft fruit, for example apples, pears, plums, peaches, almonds, cherries and berries, for example strawberries, raspberries, blackberries; legumes, for example beans, lentils, peas and soybeans; oil crops, for example oilseed rape, mustard, poppies, olives, sunflowers, coconuts, castor oil plants, cacao and peanuts; cucurbits, for example pumpkin/squash, cucumbers and melons; fibre plants, for example cotton, flax, hemp and jute; citrus fruit, for example oranges, lemons, grapefruit and tangerines; vegetables, for example spinach, lettuce, asparagus, cabbage species, carrots, onions, tomatoes, potatoes and bell peppers; Lauraceae, for example avocado, Cinnamomum, camphor, or else plants such as tobacco, nuts, coffee, aubergine, sugar cane, tea, pepper, grapevines, hops, bananas, latex plants and ornamentals, for example flowers, shrubs, deciduous trees and coniferous trees. This enumeration is no limitation.

The following plants are considered to be particularly suitable target crops: cotton, aubergine, turf, pome fruit, stone fruit, soft fruit, maize, wheat, barley, cucumber, tobacco, vines, rice, cereals, pear, beans, soybeans, oilseed rape, tomato, bell pepper, melons, cabbage, potato and apple.

Examples of trees which can be improved in accordance with the method according to the invention are: Abies sp., Eucalyptus sp., Picea sp., Pinus sp., Aesculus sp., Platanus sp., Tilia sp., Acer sp., Tsuga sp., Fraxinus sp., Sorbus sp., Betula sp., Crataegus sp., Ulmus sp., Quercus sp., Fagus sp., Salix sp., Populus sp.

Preferred trees which can be improved in accordance with the method according to the invention are: from the tree species Aesculus: A. hippocastanum, A. pariflora, A. carnea; from the tree species Platanus: P. aceriflora, P. occidentalis, P. racemosa; from the tree species Picea: P. abies; from the tree species Pinus: P. radiata, P. ponderosa, P. contorta, P. sylvestre, P. elliottii, P. montecola, P. albicaulis, P. resinosa, P. palustris, P. taeda, P. flexilis, P. jeffregi, P. baksiana, P. strobus; from the tree species Eucalyptus: E. grandis, E. globulus, E. camadentis, E. nitens, E. obliqua, E. regnans, E. pilularus.

Especially preferred trees which can be improved in accordance with the method according to the invention are: from the tree species Pinus: P. radiata, P. ponderosa, P. contorta, P. sylvestre, P. strobus; from the tree species Eucalyptus: E. grandis, E. globulus, E. camadentis.

Very particularly preferred trees which can be improved in accordance with the method according to the invention are: horse chestnut, Platanaceae, linden tree, maple tree.

The present invention can also be applied to any turf grasses, including cool-season turf grasses and warm-season turf grasses. Examples of cold-season turf grasses are bluegrasses (Poa spp.), such as Kentucky bluegrass (Poa pratensis L.), rough bluegrass (Poa trivialis L.), Canada bluegrass (Poa compressa L.), annual bluegrass (Poa annua L.), upland bluegrass (Poa glaucantha Gaudin), wood bluegrass (Poa nemoralis L.) and bulbous bluegrass (Poa bulbosa L.); bentgrasses (Agrostis spp.) such as creeping bentgrass (Agrostis palustris Huds.), colonial bentgrass (Agrostis tenuis Sibth.), velvet bentgrass (Agrostis canina L.), South German mixed bentgrass (Agrostis spp. including Agrostis tenuis Sibth., Agrostis canina L., and Agrostis palustris Huds.), and redtop (Agrostis alba L.);

fescues (Festuca spp.), such as red fescue (Festuca rubra L. spp. rubra), creeping fescue (Festuca rubra L.), chewings fescue (Festuca rubra commutata Gaud.), sheep fescue (Festuca ovina L.), hard fescue (Festuca longifolia Thuill.), hair fescue (Festucu capillata Lam.), tall fescue (Festuca arundinacea Schreb.) and meadow fescue (Festuca elanor L.);
ryegrasses (Lolium spp.), such as annual ryegrass (Lolium multiflorum Lam.), perennial ryegrass (Lolium perenne L.) and Italian ryegrass (Lolium multiflorum Lam.);
and wheatgrasses (Agropyron spp.), such as fairway wheatgrass (Agropyron cristatum (L.) Gaertn.), crested wheatgrass (Agropyron desertorum (Fisch.) Schult.) and western wheatgrass (Agropyron smithii Rydb.).

Examples of further cool-season turf grasses are beachgrass (Ammophila breviligulata Fern.), smooth bromegrass (Bromus inermis Leyss.), cattails such as timothy (Phleum pratense L.), sand cattail (Phleum subulatum L.), orchardgrass (Dactylis glomerata L.), weeping alkaligrass (Puccinellia distans (L.) Parl.) and crested dog's-tail (Cynosurus cristatus L.).

Examples of warm-season turf grasses are Bermuda grass (Cynodon spp. L. C. Rich), zoysia grass (Zoysia spp. Willd.), St. Augustine grass (Stenotaphrum secundatum Walt Kuntze), centipede grass (Eremochloa ophiuroides Munro Hack.), carpetgrass (Axonopus affinis Chase), Bahia grass (Paspalum notatum Flugge), Kikuyu grass (Pennisetum clandestinum Hochst. ex Chiov.), buffalo grass (Buchloe dactyloids (Nutt.) Engelm.), blue grama (Bouteloua gracilis (H.B.K.) Lag. ex Griffiths), seashore paspalum (Paspalum vaginatum Swartz) and sideoats grama (Bouteloua curtipendula (Michx. Torr.). Cool-season turf grasses are generally preferred for the use according to the invention. Especially preferred are bluegrass, benchgrass and redtop, fescues and ryegrasses. Bentgrass is especially preferred.

All plants and plant parts can be treated in accordance with the invention. In the present context, plants are understood as meaning all plants and plant populations, such as desired and undesired wild plants or crop plants (including naturally occurring crop plants). Crop plants can be plants which can be obtained by traditional breeding and optimization methods or by biotechnological and recombinant methods, or combinations of these methods, including the transgenic plants and including the plant varieties capable or not of being protected by Plant Breeders' Rights. Plant parts are understood as meaning all aerial and subterranean parts and organs of the plants, such as shoot, leaf, flower and root, examples which may be mentioned being leaves, needles, stalks, stems, flowers, fruiting bodies, fruits and seeds, and also roots, tubers and rhizomes. The plant parts also include crop material and vegetative and generative propagation material, for example cuttings, tubers, rhizomes, slips and seeds.

The control of animal pests by treating the seed of plants has been known for a long time and is a subject of continual improvements. Nevertheless, the treatment of seed entails a series of problems which cannot always be solved in a satisfactory manner. Thus, it is desirable to develop methods for protecting the seed and the germinating plant that remove the need for, or at least significantly reduce, the additional delivery of crop protection compositions in the course of storage, after sowing or after the emergence of the plants. It is desirable, furthermore, to optimize the amount of active ingredient employed in such a way as to provide the best-possible protection to the seed and the germinating plant from attack by animal pests, but without causing damage to the plant itself by the active ingredient employed. In particular, methods for treating seed ought also to take into consideration the intrinsic insecticidal and/or nematicidal properties of pest-resistant or pest-tolerant transgenic plants, in order to achieve optimum protection of the seed and of the germinating plant with a minimal use of crop protection compositions.

The present invention therefore also relates in particular to a method for protecting seed and germinating plants from attack by pests, by treating the seed with a compound of formula (I).

The invention likewise relates to the use of the compound of formula (I) for treating seed for the purpose of protecting the seed and the resultant plant against animal pests.

The invention relates, furthermore, to seed which for protection against animal pests has been treated with a compound of formula (I).

Furthermore, the invention relates to seed which, following treatment with a compound of formula (I) of the invention, is subjected to a film-coating process in order to prevent dust abrasion of the seed.

One of the advantages of the present invention is that, owing to the particular systemic properties of the compositions of the invention, the treatment of the seed with these compositions provides protection from animal pests not only to the seed itself but also to the plants originating from the seed, after they have emerged. In this way, it may not be necessary to treat the crop directly at the time of sowing or shortly thereafter.

A further advantage is to be seen in the fact that, through the treatment of the seed with a compound of formula (I) of the invention, germination and emergence of the treated seed may be promoted.

It is likewise considered to be advantageous that compound of formula (I) may also be used, in particular, on transgenic seed.

It is also stated that a compound of formula (I) may be used in combination with agents of the signalling technology, as a result of which, for example, colonization with symbionts is improved, such as rhizobia, mycorrhiza and/or endophytic bacteria, for example, is enhanced, and/or nitrogen fixation is optimized.

The compositions of the invention are suitable for protecting seed of any variety of plant which is used in agriculture, in greenhouses, in forestry or in horticulture. More particularly, the seed in question is that of cereals (e.g. wheat, barley, rye, oats and millet), maize, cotton, soybeans, rice, potatoes, sunflower, coffee, tobacco, canola, oilseed rape, beets (e.g. sugar beet and fodder beet), peanuts, vegetables (e.g. tomato, cucumber, bean, brassicas, onions and lettuce), fruit plants, lawns and ornamentals. Particularly important is the treatment of the seed of cereals (such as wheat, barley, rye and oats) maize, soybeans, cotton, canola, oilseed rape and rice.

As already mentioned above, the treatment of transgenic seed with a compound of formula (I) is particularly important. The seed in question here is that of plants which generally contain at least one heterologous gene that controls the expression of a polypeptide having, in particular, insecticidal and/or nematicidal properties. These heterologous genes in transgenic seed may come from microorganisms such as Bacillus, Rhizobium, Pseudomonas, Serratia, Trichoderma, Clavibacter, Glomus or Gliocladium. The present invention is particularly suitable for the treatment of transgenic seed which contains at least one heterologous gene from Bacillus sp. With particular preference, the heterologous gene in question comes from Bacillus thuringiensis.

For the purposes of the present invention, the compound of formula (I) of the invention is applied alone or in a suitable formulation to the seed. The seed is preferably treated in a condition in which its stability is such that no damage occurs in the course of the treatment. Generally speaking, the seed may be treated at any point in time between harvesting and sowing. Typically, seed is used which has been separated from the plant and has had cobs, hulls, stems, husks, hair or pulp removed. Thus, for example, seed may be used that has been harvested, cleaned and dried to a moisture content of less than 15% by weight. Alternatively, seed can also be used that after drying has been treated with water, for example, and then dried again.

When treating seed it is necessary, generally speaking, to ensure that the amount of the composition of the invention, and/or of other additives, that is applied to the seed is selected such that the germination of the seed is not adversely affected, and/or that the plant which emerges from the seed is not damaged. This is the case in particular with active ingredients which may exhibit phytotoxic effects at certain application rates.

The compositions of the invention can be applied directly, in other words without comprising further components and without having been diluted. As a general rule, it is preferable to apply the compositions in the form of a suitable formulation to the seed. Suitable formulations and methods for seed treatment are known to the skilled person and are described in, for example, the following documents: U.S. Pat. No. 4,272,417 A, U.S. Pat. No. 4,245,432 A, U.S. Pat. No. 4,808,430 A, U.S. Pat. No. 5,876,739 A, US 2003/0176428 A1, WO 2002/080675 A1, WO 2002/028186 A2.

The compound of formula (I) which can be used in accordance with the invention may be converted into the customary seed-dressing formulations, such as solutions, emulsions, suspensions, powders, foams, slurries or other coating compositions for seed, and also ULV formulations.

These formulations are prepared in a known manner, by mixing the compound of formula (I) with customary adjuvants, such as, for example, customary extenders and also solvents or diluents, colorants, wetters, dispersants, emulsifiers, antifoams, preservatives, secondary thickeners, stickers, gibberellins, and also water.

Colorants which may be present in the seed-dressing formulations which can be used in accordance with the invention include all colorants which are customary for such purposes. In this context it is possible to use not only pigments, which are of low solubility in water, but also water-soluble dyes. Examples include the colorants known under the designations Rhodamin B, C.I. Pigment Red 112 and C.I. Solvent Red 1.

Wetters which may be present in the seed-dressing formulations which can be used in accordance with the invention include all of the substances which promote wetting and which are customary in the formulation of active agrochemical ingredients. Use may be made preferably of alkylnaphthalenesulphonates, such as diisopropyl- or diisobutyl-naphthalenesulphonates.

Dispersants and/or emulsifiers which may be present in the seed-dressing formulations which can be used in accordance with the invention include all of the nonionic, anionic and cationic dispersants that are customary in the formulation of active agrochemical ingredients. Use may be made preferably of nonionic or anionic dispersants or of mixtures of nonionic or anionic dispersants. Suitable nonionic dispersants are, in particular, ethylene oxide-propylene oxide block polymers, alkylphenol polyglycol ethers and also tristryrylphenol polyglycol ethers, and the phosphated or sulphated derivatives of these. Suitable anionic dispersants are, in particular, lignosulphonates, salts of polyacrylic acid, and arylsulphonate-formaldehyde condensates.

Antifoams which may be present in the seed-dressing formulations which can be used in accordance with the invention include all of the foam inhibitors that are customary in the formulation of active agrochemical ingredients. Use may be made preferably of silicone antifoams and magnesium stearate.

Preservatives which may be present in the seed-dressing formulations which can be used in accordance with the invention include all of the substances which can be employed for such purposes in agrochemical compositions. Examples include dichlorophen and benzyl alcohol hemiformal.

Secondary thickeners which may be present in the seed-dressing formulations which can be used in accordance with the invention include all substances which can be used for such purposes in agrochemical compositions. Those contemplated with preference include cellulose derivatives, acrylic acid derivatives, xanthan, modified clays and highly disperse silica.

Stickers which may be present in the seed-dressing formulations which can be used in accordance with the invention include all customary binders which can be used in seed-dressing products. Preferred mention may be made of polyvinylpyrrolidone, polyvinyl acetate, polyvinyl alcohol and tylose.

Gibberellins which may be present in the seed-dressing formulations which can be used in accordance with the invention include preferably the gibberellins A1, A3 (=gibberellic acid), A4 and A7, with gibberellic acid being used with particular preference. The gibberellins are known (cf. R. Wegler, “Chemie der Pflanzenschutz-und Schädlingsbekämpfungsmittel”, Volume 2, Springer Verlag, 1970, pp. 401-412).

The seed-dressing formulations which can be used in accordance with the invention may be used, either directly or after prior dilution with water, to treat seed of any of a wide variety of types. Accordingly, the concentrates or the preparations obtainable from them by dilution with water may be employed to dress the seed of cereals, such as wheat, barley, rye, oats and triticale, and also the seed of maize, rice, oilseed rape, peas, beans, cotton, sunflowers and beets, or else the seed of any of a very wide variety of vegetables. The seed-dressing formulations which can be used in accordance with the invention, or their diluted preparations, may also be used to dress seed of transgenic plants. In that case, additional synergistic effects may occur in interaction with the substances formed through expression.

For the treatment of seed with the seed-dressing formulations which can be used in accordance with the invention, or with the preparations produced from them by addition of water, suitable mixing equipment includes all such equipment which can typically be employed for seed dressing. More particularly, the procedure when carrying out seed dressing is to place the seed in a mixer, to add the particular desired amount of seed-dressing formulations, either as such or following dilution with water beforehand, and to carry out mixing until the distribution of the formulation on the seed is uniform. This may be followed by a drying operation.

The application rate of the seed-dressing formulations which can be used in accordance with the invention may be varied within a relatively wide range. It is guided by the particular amount of the compound of formula (I) in the formulations, and by the seed. The application rates with regard to the compound of formula (I) are situated generally at between 0.001 and 50 g per kilogram of seed, preferably between 0.01 and 15 g per kilogram of seed.

As has already been mentioned above, all plants and their parts may be treated in accordance with the invention. In a preferred embodiment, plant species and plant varieties, and their parts, which grow wild or which are obtained by traditional biological breeding methods such as hybridization or protoplast fusion are treated. In a further preferred embodiment, transgenic plants and plant varieties which have been obtained by recombinant methods, if appropriate in combination with traditional methods (genetically modified organisms), and their parts are treated. The term “parts” or “parts of plants” or “plant parts” has been explained hereinabove. Plants of the plant varieties which are in each case commercially available or in use are especially preferably treated in accordance with the invention. Plant varieties are understood as meaning plants with novel traits which have been bred both by traditional breeding, by mutagenesis or by recombinant DNA techniques. They may take the form of varieties, races, biotypes and genotypes.

The method of treatment according to the invention can be used in the treatment of genetically modified organisms (GMOs), e.g. plants or seeds. Genetically modified plants (or transgenic plants) are plants of which a heterologous gene has been stably integrated into genome. The expression “heterologous gene” essentially means a gene which is provided or assembled outside the plant and when introduced in the nuclear, chloroplastic or mitochondrial genome gives the transformed plant new or improved agronomic or other properties by expressing a protein or polypeptide of interest or by downregulating or silencing other gene(s) which are present in the plant (using for example, antisense technology, cosuppression technology, RNA interference-RNAi-technology or microRNA-miRNA-technology). A heterologous gene that is located in the genome is also called a transgene. A transgene that is defined by its particular location in the plant genome is called a transformation or transgenic event.

Plants and plant cultivars which are preferably to be treated according to the invention include all plants which have genetic material which impart particularly advantageous, useful traits to these plants (whether obtained by breeding and/or biotechnological means).

Plants and plant cultivars which are also preferably to be treated according to the invention are resistant against one or more biotic stresses, i.e. said plants show a better defense against animal and microbial pests, such as against nematodes, insects, mites, phytopathogenic fungi, bacteria, viruses and/or viroids.

Examples of nematode or insect resistant plants are described in e.g. U.S. patent application Ser. Nos. 11/765,491, 11/765,494, 10/926,819, 10/782,020, 12/032,479, 10/783,417, 10/782,096, 11/657,964, 12/192,904, 11/396,808, 12/166,253, 12/166,239, 12/166,124, 12/166,209, 11/762,886, 12/364,335, 11/763,947, 12/252,453, 12/209,354, 12/491,396, 12/497,221, 12/644,632, 12/646,004, 12/701,058, 12/718,059, 12/721,595, 12/638,591, and in WO 11/002992, WO 11/014749, WO 11/103247, WO 11/103248, WO 12/135436, WO 12/135501.

Plants and plant cultivars which may also be treated according to the invention are those plants which are resistant to one or more abiotic stresses. Abiotic stress conditions may include, for example, drought, cold temperature exposure, heat exposure, osmotic stress, flooding, increased soil salinity, increased mineral exposure, ozone exposure, high light exposure, limited availability of nitrogen nutrients, limited availability of phosphorus nutrients, shade avoidance.

Plants and plant cultivars which may also be treated according to the invention, are those plants characterized by enhanced yield characteristics. Increased yield in said plants can be the result of, for example, improved plant physiology, growth and development, such as water use efficiency, water retention efficiency, improved nitrogen use, enhanced carbon assimilation, improved photosynthesis, increased germination efficiency and accelerated maturation. Yield can furthermore be affected by improved plant architecture (under stress and non-stress conditions), including but not limited to, early flowering, flowering control for hybrid seed production, seedling vigor, plant size, internode number and distance, root growth, seed size, fruit size, pod size, pod or ear number, seed number per pod or ear, seed mass, enhanced seed filling, reduced seed dispersal, reduced pod dehiscence and lodging resistance. Further yield traits include seed composition, such as carbohydrate content, protein content, oil content and composition, nutritional value, reduction in anti-nutritional compounds, improved processability and better storage stability.

Plants that may be treated according to the invention are hybrid plants that already express the characteristic of heterosis or hybrid vigor which results in generally higher yield, vigor, health and resistance towards biotic and abiotic stresses). Such plants are typically made by crossing an inbred male-sterile parent line (the female parent) with another inbred male-fertile parent line (the male parent). Hybrid seed is typically harvested from the male sterile plants and sold to growers. Male sterile plants can sometimes (e.g. in corn) be produced by detasseling, i.e. the mechanical removal of the male reproductive organs (or males flowers) but, more typically, male sterility is the result of genetic determinants in the plant genome. In that case, and especially when seed is the desired product to be harvested from the hybrid plants it is typically useful to ensure that male fertility in the hybrid plants is fully restored. This can be accomplished by ensuring that the male parents have appropriate fertility restorer genes which are capable of restoring the male fertility in hybrid plants that contain the genetic determinants responsible for male-sterility. Genetic determinants for male sterility may be located in the cytoplasm. Examples of cytoplasmic male sterility (CMS) were for instance described in Brassica species (WO 92/05251, WO 95/09910, WO 98/27806, WO 05/002324, WO 06/021972 and U.S. Pat. No. 6,229,072). However, genetic determinants for male sterility can also be located in the nuclear genome. Male sterile plants can also be obtained by plant biotechnology methods such as genetic engineering. A particularly useful means of obtaining male-sterile plants is described in WO 89/10396 in which, for example, a ribonuclease such as barnase is selectively expressed in the tapetum cells in the stamens. Fertility can then be restored by expression in the tapetum cells of a ribonuclease inhibitor such as barstar (e.g. WO 91/02069).

Plants or plant cultivars (obtained by plant biotechnology methods such as genetic engineering) which may be treated according to the invention are herbicide-tolerant plants, i.e. plants made tolerant to one or more given herbicides. Such plants can be obtained either by genetic transformation, or by selection of plants containing a mutation imparting such herbicide tolerance.

Herbicide-resistant plants are for example glyphosate-tolerant plants, i.e. plants made tolerant to the herbicide glyphosate or salts thereof. Plants can be made tolerant to glyphosate through different means. For example, glyphosate-tolerant plants can be obtained by transforming the plant with a gene encoding the enzyme 5-enol-pyruvylshikimate-3-phosphate synthase (EPSPS). Examples of such EPSPS genes are the AroA gene (mutant CT7) of the bacterium Salmonella typhimurium (Science 1983, 221, 370-371), the CP4 gene of the bacterium Agrobacterium sp. (Curr. Topics Plant Physiol. 1992, 7, 139-145), the genes encoding a Petunia EPSPS (Science 1986, 233, 478-481), a Tomato EPSPS (J. Biol. Chem. 1988, 263, 4280-4289), or an Eleusine EPSPS (WO 01/66704). It can also be a mutated EPSPS as described in for example EP0837944, WO 0066746, WO 00/66747 or WO 02/26995, WO 11/000498. Glyphosate-tolerant plants can also be obtained by expressing a gene that encodes a glyphosate oxido-reductase enzyme as described in U.S. Pat. No. 5,776,760 and U.S. Pat. No. 5,463,175. Glyphosate-tolerant plants can also be obtained by expressing a gene that encodes a glyphosate acetyl transferase enzyme as described in for example WO 02/036782, WO 03/092360, WO05/012515 and WO 07/024782. Glyphosate-tolerant plants can also be obtained by selecting plants containing naturally-occurring mutations of the above-mentioned genes, as described in for example WO 01/024615 or WO 03/013226. Plants expressing EPSPS genes that confer glyphosate tolerance are described in e.g. U.S. patent application Ser. Nos. 11/517,991, 10/739,610, 12/139,408, 12/352,532, 11/312,866, 11/315,678, 12/421,292, 11/400,598, 11/651,752, 11/681,285, 11/605,824, 12/468,205, 11/760,570, 11/762,526, 11/769,327, 11/769,255, 11/943,801 or 12/362,774. Plants comprising other genes that confer glyphosate tolerance, such as decarboxylase genes, are described in e.g. U.S. patent application Ser. Nos. 11/588,811, 11/185,342, 12/364,724, 11/185,560 or 12/423,926.

Other herbicide resistant plants are for example plants that are made tolerant to herbicides inhibiting the enzyme glutamine synthase, such as bialaphos, phosphinothricin or glufosinate. Such plants can be obtained by expressing an enzyme detoxifying the herbicide or a mutant glutamine synthase enzyme that is resistant to inhibition, e.g. described in U.S. patent application Ser. No. 11/760,602. One such efficient detoxifying enzyme is an enzyme encoding a phosphinothricin acetyltransferase (such as the bar or pat protein from Streptomyces species). Plants expressing an exogenous phosphinothricin acetyltransferase are for example described in U.S. Pat. Nos. 5,561,236; 5,648,477; 5,646,024; 5,273,894; 5,637,489; 5,276,268; 5,739,082; 5,908,810 and 7,112,665.

Further herbicide-tolerant plants are also plants that are made tolerant to the herbicides inhibiting the enzyme hydroxyphenylpyruvatedioxygenase (HPPD). HPPD is an enzyme that catalyze the reaction in which para-hydroxyphenylpyruvate (HPP) is transformed into homogentisate. Plants tolerant to HPPD-inhibitors can be transformed with a gene encoding a naturally-occurring resistant HPPD enzyme, or a gene encoding a mutated or chimeric HPPD enzyme as described in WO 96/38567, WO 99/24585, WO 99/24586, WO 09/144079, WO 02/046387, U.S. Pat. No. 6,768,044, WO 11/076877, WO 11/076882, WO 11/076885, WO 11/076889 or WO 11/076892. Tolerance to HPPD-inhibitors can also be obtained by transforming plants with genes encoding certain enzymes enabling the formation of homogentisate despite the inhibition of the native HPPD enzyme by the HPPD-inhibitor. Such plants and genes are described in WO 99/34008 and WO 02/36787. Tolerance of plants to HPPD inhibitors can also be improved by transforming plants with a gene encoding an enzyme having prephenate deshydrogenase (PDH) activity in addition to a gene encoding an HPPD-tolerant enzyme, as described in WO 04/024928. Further, plants can be made more tolerant to HPPD-inhibitor herbicides by adding into their genome a gene encoding an enzyme capable of metabolizing or degrading HPPD inhibitors, such as the CYP450 enzymes shown in WO 07/103567 and WO 08/150473.

Still further herbicide resistant plants are plants that are made tolerant to acetolactate synthase (ALS) inhibitors. Known ALS-inhibitors include, for example, sulfonylurea, imidazolinone, triazolopyrimidines, pyrimidinyoxy-(thio)benzoates, and/or sulfonylaminocarbonyltriazolinone herbicides. Different mutations in the ALS enzyme (also known as acetohydroxyacid synthase, AHAS) are known to confer tolerance to different herbicides and groups of herbicides, as described for example in Tranel and Wright (Weed Science 2002, 50, 700-712), but also, in U.S. Pat. Nos. 5,605,011, 5,378,824, 5,141,870, and 5,013,659. The production of sulfonylurea-tolerant plants and imidazolinone-tolerant plants is described in U.S. Pat. Nos. 5,605,011; 5,013,659; 5,141,870; 5,767,361; 5,731,180; 5,304,732; 4,761,373; 5,331,107; 5,928,937; and 5,378,824; and WO 96/33270. Other imidazolinone-tolerant plants are also described in for example WO 04/040012, WO 04/106529, WO 05/020673, WO 05/093093, WO 06/007373, WO 06/015376, WO 06/024351, and WO 06/060634. Further sulfonylurea- and imidazolinone-tolerant plants are also described in for example WO 07/024782, WO 2011/076345, WO 2012058223, WO 2012150335 and U.S. Patent Application 61/288,958.

Other plants tolerant to imidazolinone and/or sulfonylurea can be obtained by induced mutagenesis, selection in cell cultures in the presence of the herbicide or mutation breeding as described for example for soybeans in U.S. Pat. No. 5,084,082, for rice in WO 97/41218, for sugar beet in U.S. Pat. No. 5,773,702 and WO 99/057965, for lettuce in U.S. Pat. No. 5,198,599, or for sunflower in WO 01/065922.

Plants or plant cultivars (obtained by plant biotechnology methods such as genetic engineering) which may also be treated according to the invention are insect-resistant transgenic plants, i.e. plants made resistant to attack by certain target insects. Such plants can be obtained by genetic transformation, or by selection of plants containing a mutation imparting such insect resistance.

An “insect-resistant transgenic plant”, as used herein, includes any plant containing at least one transgene comprising a coding sequence encoding:
1) an insecticidal crystal protein from Bacillus thuringiensis or an insecticidal portion thereof, such as the insecticidal crystal proteins listed by Crickmore et al. (Microbiology and Molecular Biology Reviews 1998, 62, 807-813), updated by Crickmore et al. (2005) at the Bacillus thuringiensis toxin nomenclature, online at: http://www.lifesci.sussex.ac.uk/Home/Neil_Crickmore/Bt/), or insecticidal portions thereof, e.g., proteins of the Cry protein classes Cry1Ab, Cry1Ac, Cry1B, Cry1C, Cry1D, Cry1F, Cry2Ab, Cry3Aa, or Cry3Bb or insecticidal portions thereof (e.g. EP-A 1 999 141 and WO 07/107302), or such proteins encoded by synthetic genes as e.g. described in and U.S. patent application Ser. No. 12/249,016; or
2) a crystal protein from Bacillus thuringiensis or a portion thereof which is insecticidal in the presence of a second other crystal protein from Bacillus thuringiensis or a portion thereof, such as the binary toxin made up of the Cry34 and Cry35 crystal proteins (Nat. Biotechnol. 2001, 19, 668-72; Applied Environm. Microbiol. 2006, 71, 1765-1774) or the binary toxin made up of the Cry1A or Cry1F proteins and the Cry2Aa or Cry2Ab or Cry2Ae proteins (U.S. patent application Ser. No. 12/214,022 and EP-A 2 300 618); or
3) a hybrid insecticidal protein comprising parts of different insecticidal crystal proteins from Bacillus thuringiensis, such as a hybrid of the proteins of 1) above or a hybrid of the proteins of 2) above, e.g., the Cry1A.105 protein produced by corn event MON89034 (WO 07/027777); or
4) a protein of any one of 1) to 3) above wherein some, particularly 1 to 10, amino acids have been replaced by another amino acid to obtain a higher insecticidal activity to a target insect species, and/or to expand the range of target insect species affected, and/or because of changes introduced into the encoding DNA during cloning or transformation, such as the Cry3Bb1 protein in corn events MON863 or MON88017, or the Cry3A protein in corn event MIR604; or
5) an insecticidal secreted protein from Bacillus thuringiensis or Bacillus cereus, or an insecticidal portion thereof, such as the vegetative insecticidal (VIP) proteins listed at: http://www.lifesci.sussex.ac.uk/home/Neil_Crickmore/Bt/vip.html, e.g., proteins from the VIP3Aa protein class; or
6) a secreted protein from Bacillus thuringiensis or Bacillus cereus which is insecticidal in the presence of a second secreted protein from Bacillus thuringiensis or B. cereus, such as the binary toxin made up of the VIP1A and VIP2A proteins (WO 94/21795); or
7) a hybrid insecticidal protein comprising parts from different secreted proteins from Bacillus thuringiensis or Bacillus cereus, such as a hybrid of the proteins in 1) above or a hybrid of the proteins in 2) above; or
8) a protein of any one of 5) to 7) above wherein some, particularly 1 to 10, amino acids have been replaced by another amino acid to obtain a higher insecticidal activity to a target insect species, and/or to expand the range of target insect species affected, and/or because of changes introduced into the encoding DNA during cloning or transformation (while still encoding an insecticidal protein), such as the VIP3Aa protein in cotton event COT102; or
9) a secreted protein from Bacillus thuringiensis or Bacillus cereus which is insecticidal in the presence of a crystal protein from Bacillus thuringiensis, such as the binary toxin made up of VIP3 and Cry1A or Cry1F (U.S. Patent Applications 61/126,083 and 61/195019), or the binary toxin made up of the VIP3 protein and the Cry2Aa or Cry2Ab or Cry2Ae proteins (U.S. patent application Ser. No. 12/214,022 and EP-A 2 300 618).
10) a protein of 9) above wherein some, particularly 1 to 10, amino acids have been replaced by another amino acid to obtain a higher insecticidal activity to a target insect species, and/or to expand the range of target insect species affected, and/or because of changes introduced into the encoding DNA during cloning or transformation (while still encoding an insecticidal protein)

Of course, an insect-resistant transgenic plant, as used herein, also includes any plant comprising a combination of genes encoding the proteins of any one of the above classes 1 to 10. In one embodiment, an insect-resistant plant contains more than one transgene encoding a protein of any one of the above classes 1 to 10, to expand the range of target insect species affected when using different proteins directed at different target insect species, or to delay insect resistance development to the plants by using different proteins insecticidal to the same target insect species but having a different mode of action, such as binding to different receptor binding sites in the insect.

An “insect-resistant transgenic plant”, as used herein, further includes any plant containing at least one transgene comprising a sequence producing upon expression a double-stranded RNA which upon ingestion by a plant insect pest inhibits the growth of this insect pest, as described e.g. in WO 07/080126, WO 06/129204, WO 07/074405, WO 07/080127 and WO 07/035650.

Plants or plant cultivars (obtained by plant biotechnology methods such as genetic engineering) which may also be treated according to the invention are tolerant to abiotic stresses. Such plants can be obtained by genetic transformation, or by selection of plants containing a mutation imparting such stress resistance. Particularly useful stress tolerance plants include:
1) plants which contain a transgene capable of reducing the expression and/or the activity of poly(ADP-ribose) polymerase (PARP) gene in the plant cells or plants as described in WO 00/04173, WO 06/045633, EP-A 1 807 519, or EP-A 2 018 431.
2) plants which contain a stress tolerance enhancing transgene capable of reducing the expression and/or the activity of the PARG encoding genes of the plants or plants cells, as described e.g. in WO 04/090140.
3) plants which contain a stress tolerance enhancing transgene coding for a plant-functional enzyme of the nicotineamide adenine dinucleotide salvage synthesis pathway including nicotinamidase, nicotinate phosphoribosyltransferase, nicotinic acid mononucleotide adenyl transferase, nicotinamide adenine dinucleotide synthetase or nicotine amide phosphorybosyltransferase as described e.g. in EP-A 1 794 306, WO 06/133827, WO 07/107326, EP-A 1 999 263, or WO 07/107326.

Plants or plant cultivars (obtained by plant biotechnology methods such as genetic engineering) which may also be treated according to the invention show altered quantity, quality and/or storage-stability of the harvested product and/or altered properties of specific ingredients of the harvested product such as:
1) transgenic plants which synthesize a modified starch, which in its physical-chemical characteristics, in particular the amylose content or the amylose/amylopectin ratio, the degree of branching, the average chain length, the side chain distribution, the viscosity behaviour, the gelling strength, the starch grain size and/or the starch grain morphology, is changed in comparison with the synthesised starch in wild type plant cells or plants, so that this is better suited for special applications. Said transgenic plants synthesizing a modified starch are disclosed, for example, in EP-A 0 571 427, WO 95/04826, EP-A 0 719 338, WO 96/15248, WO 96/19581, WO 96/27674, WO 97/11188, WO 97/26362, WO 97/32985, WO 97/42328, WO 97/44472, WO 97/45545, WO 98/27212, WO 98/40503, WO 99/58688, WO 99/58690, WO 99/58654, WO 00/08184, WO 00/08185, WO 00/08175, WO 00/28052, WO 00/77229, WO 01/12782, WO 01/12826, WO 02/101059, WO 03/071860, WO 04/056999, WO 05/030942, WO 05/030941, WO 05/095632, WO 05/095617, WO 05/095619, WO 2005/095618, WO 05/123927, WO 06/018319, WO 06/103107, WO 06/108702, WO 07/009823, WO 00/22140, WO 06/063862, WO 06/072603, WO 02/034923, WO 08/017518, WO 08/080630, WO 08/080631, WO 08/090008, WO 01/14569, WO 02/79410, WO 03/33540, WO 04/078983, WO 01/19975, WO 95/26407, WO 96/34968, WO 98/20145, WO 99/12950, WO 99/66050, WO 99/53072, U.S. Pat. No. 6,734,341, WO 00/11192, WO 98/22604, WO 98/32326, WO 01/98509, WO 01/98509, WO 05/002359, U.S. Pat. No. 5,824,790, U.S. Pat. No. 6,013,861, WO 94/04693, WO 94/09144, WO 94/11520, WO 95/35026, WO 97/20936, WO 10/012796, WO 10/003701,
2) transgenic plants which synthesize non starch carbohydrate polymers or which synthesize non starch carbohydrate polymers with altered properties in comparison to wild type plants without genetic modification. Examples are plants producing polyfructose, especially of the inulin and levan-type, as disclosed in EP-A 0 663 956, WO 96/01904, WO 96/21023, WO 98/39460, and WO 99/24593, plants producing alpha-1,4-glucans as disclosed in WO 95/31553, US 2002031826, U.S. Pat. No. 6,284,479, U.S. Pat. No. 5,712,107, WO 97/47806, WO 97/47807, WO 97/47808 and WO 00/14249, plants producing alpha-1,6 branched alpha-1,4-glucans, as disclosed in WO 00/73422, plants producing alternan, as disclosed in e.g. WO 00/47727, WO 00/73422, U.S. Pat. No. 5,908,975 and EP-A 0 728 213,
3) transgenic plants which produce hyaluronan, as for example disclosed in WO 06/032538, WO 07/039314, WO 07/039315, WO 07/039316, JP-A 2006-304779, and WO 05/012529.
4) transgenic plants or hybrid plants, such as onions with characteristics such as ‘high soluble solids content’, ‘low pungency’ (LP) and/or ‘long storage’ (LS), as described in U.S. patent application Ser. No. 12/020,360.
5) Transgenic plants displaying an increase yield as for example disclosed in WO 11/095528

Plants or plant cultivars (that can be obtained by plant biotechnology methods such as genetic engineering) which may also be treated according to the invention are plants, such as cotton plants, with altered fiber characteristics. Such plants can be obtained by genetic transformation, or by selection of plants contain a mutation imparting such altered fiber characteristics and include:
a) Plants, such as cotton plants, containing an altered form of cellulose synthase genes as described in WO 98/00549.
b) Plants, such as cotton plants, containing an altered form of rsw2 or rsw3 homologous nucleic acids as described in WO 04/053219.
c) Plants, such as cotton plants, with increased expression of sucrose phosphate synthase as described in WO 01/17333.
d) Plants, such as cotton plants, with increased expression of sucrose synthase as described in WO 02/45485.
e) Plants, such as cotton plants, wherein the timing of the plasmodesmatal gating at the basis of the fiber cell is altered, e.g. through downregulation of fiber-selective β-1,3-glucanase as described in WO 05/017157, or as described in WO 09/143995.
f) Plants, such as cotton plants, having fibers with altered reactivity, e.g. through the expression of N-acetylglucosaminetransferase gene including nodC and chitin synthase genes as described in WO 06/136351, WO 11/089021, WO 11/089021, WO 12/074868.

Plants or plant cultivars (that can be obtained by plant biotechnology methods such as genetic engineering) which may also be treated according to the invention are plants, such as oilseed rape or related Brassica plants, with altered oil profile characteristics. Such plants can be obtained by genetic transformation, or by selection of plants contain a mutation imparting such altered oil profile characteristics and include:
a) Plants, such as oilseed rape plants, producing oil having a high oleic acid content as described e.g. in U.S. Pat. No. 5,969,169, U.S. Pat. No. 5,840,946 or U.S. Pat. No. 6,323,392 or U.S. Pat. No. 6,063,947
b) Plants such as oilseed rape plants, producing oil having a low linolenic acid content as described in U.S. Pat. No. 6,270,828, U.S. Pat. No. 6,169,190, U.S. Pat. No. 5,965,755 or WO 11/060946
c) Plant such as oilseed rape plants, producing oil having a low level of saturated fatty acids as described e.g. in U.S. Pat. No. 5,434,283 or U.S. patent application Ser. No. 12/668,303
d) Plants such as oilseed rape plants, producing oil having an alter glucosinolate content as described in WO 2012075426.

Plants or plant cultivars (that can be obtained by plant biotechnology methods such as genetic engineering) which may also be treated according to the invention are plants, such as oilseed rape or related Brassica plants, with altered seed shattering characteristics. Such plants can be obtained by genetic transformation, or by selection of plants contain a mutation imparting such altered seed shattering characteristics and include plants such as oilseed rape plants with delayed or reduced seed shattering as described in WO 2009/068313 and WO 2010/006732, WO 2012090499.

Plants or plant cultivars (that can be obtained by plant biotechnology methods such as genetic engineering) which may also be treated according to the invention are plants, such as Tobacco plants, with altered post-translational protein modification patterns, for example as described in WO 10/121818 and WO 10/145846.

Particularly useful transgenic plants which may be treated according to the invention are plants containing transformation events, or combination of transformation events, that are the subject of petitions for non-regulated status, in the United States of America, to the Animal and Plant Health Inspection Service (APHIS) of the United States Department of Agriculture (USDA) whether such petitions are granted or are still pending. At any time this information is readily available from APHIS (4700 River Road, Riverdale, Md. 20737, USA), for instance on its internet site (URL http://www.aphis.usda.gov/brs/not_reg.html).

Additional particularly useful plants containing single transformation events or combinations of transformation events are listed for example in the databases from various national or regional regulatory agencies (see for example http://gmoinfo.jrc.it/gmp_browse.aspx and http://www.cera-gmc.org/?action=gm_crop_database).

Particularly useful transgenic plants which may be treated according to the invention are plants containing transformation events, or a combination of transformation events, and that are listed for example in the databases for various national or regional regulatory agencies including Event 531/PV-GHBK04 (cotton, insect control, described in WO 2002/040677), Event 1143-14A (cotton, insect control, not deposited, described in WO 06/128569); Event 1143-51B (cotton, insect control, not deposited, described in WO 06/128570); Event 1445 (cotton, herbicide tolerance, not deposited, described in US-A 2002-120964 or WO 02/034946 Event 17053 (rice, herbicide tolerance, deposited as PTA-9843, described in WO 10/117737); Event 17314 (rice, herbicide tolerance, deposited as PTA-9844, described in WO 10/117735); Event 281-24-236 (cotton, insect control-herbicide tolerance, deposited as PTA-6233, described in WO 05/103266 or US-A 2005-216969); Event 3006-210-23 (cotton, insect control-herbicide tolerance, deposited as PTA-6233, described in US-A 2007-143876 or WO 05/103266); Event 3272 (corn, quality trait, deposited as PTA-9972, described in WO 06/098952 or US-A 2006-230473); Event 33391 (wheat, herbicide tolerance, deposited as PTA-2347, described in WO 2002/027004), Event 40416 (corn, insect control-herbicide tolerance, deposited as ATCC PTA-11508, described in WO 11/075593); Event 43A47 (corn, insect control-herbicide tolerance, deposited as ATCC PTA-11509, described in WO 11/075595); Event 5307 (corn, insect control, deposited as ATCC PTA-9561, described in WO 10/077816); Event ASR-368 (bent grass, herbicide tolerance, deposited as ATCC PTA-4816, described in US-A 2006-162007 or WO 04/053062); Event B16 (corn, herbicide tolerance, not deposited, described in US-A 2003-126634); Event BPS-CV127-9 (soybean, herbicide tolerance, deposited as NCIMB No. 41603, described in WO 10/080829); Event BLR1 (oilseed rape, restoration of male sterility, deposited as NCIMB 41193, described in WO 2005/074671), Event CE43-67B (cotton, insect control, deposited as DSM ACC2724, described in US-A 2009-217423 or WO 06/128573); Event CE44-69D (cotton, insect control, not deposited, described in US-A 2010-0024077); Event CE44-69D (cotton, insect control, not deposited, described in WO 06/128571); Event CE46-02A (cotton, insect control, not deposited, described in WO 06/128572); Event COT102 (cotton, insect control, not deposited, described in US-A 2006-130175 or WO 04/039986); Event COT202 (cotton, insect control, not deposited, described in US-A 2007-067868 or WO 05/054479); Event COT203 (cotton, insect control, not deposited, described in WO 05/054480); Event DAS21606-3/1606 (soybean, herbicide tolerance, deposited as PTA-11028, described in WO 012/033794), Event DAS40278 (corn, herbicide tolerance, deposited as ATCC PTA-10244, described in WO 11/022469); Event DAS-44406-6/pDAB8264.44.06.1 (soybean, herbicide tolerance, deposited as PTA-11336, described in WO 2012/075426), Event DAS-14536-7/pDAB8291.45.36.2 (soybean, herbicide tolerance, deposited as PTA-11335, described in WO 2012/075429), Event DAS-59122-7 (corn, insect control-herbicide tolerance, deposited as ATCC PTA 11384, described in US-A 2006-070139); Event DAS-59132 (corn, insect control-herbicide tolerance, not deposited, described in WO 09/100188); Event DAS68416 (soybean, herbicide tolerance, deposited as ATCC PTA-10442, described in WO 11/066384 or WO 11/066360); Event DP-098140-6 (corn, herbicide tolerance, deposited as ATCC PTA-8296, described in US-A 2009-137395 or WO 08/112019); Event DP-305423-1 (soybean, quality trait, not deposited, described in US-A 2008-312082 or WO 08/054747); Event DP-32138-1 (corn, hybridization system, deposited as ATCC PTA-9158, described in US-A 2009-0210970 or WO 09/103049); Event DP-356043-5 (soybean, herbicide tolerance, deposited as ATCC PTA-8287, described in US-A 2010-0184079 or WO 08/002872); Event EE-1 (brinjal, insect control, not deposited, described in WO 07/091277); Event FI117 (corn, herbicide tolerance, deposited as ATCC 209031, described in US-A 2006-059581 or WO 98/044140); Event FG72 (soybean, herbicide tolerance, deposited as PTA-11041, described in WO 2011/063413), Event GA21 (corn, herbicide tolerance, deposited as ATCC 209033, described in US-A 2005-086719 or WO 98/044140); Event GG25 (corn, herbicide tolerance, deposited as ATCC 209032, described in US-A 2005-188434 or WO 98/044140); Event GHB119 (cotton, insect control-herbicide tolerance, deposited as ATCC PTA-8398, described in WO 08/151780); Event GHB614 (cotton, herbicide tolerance, deposited as ATCC PTA-6878, described in US-A 2010-050282 or WO 07/017186); Event GJ11 (corn, herbicide tolerance, deposited as ATCC 209030, described in US-A 2005-188434 or WO 98/044140); Event GM RZ13 (sugar beet, virus resistance, deposited as NCIMB-41601, described in WO 10/076212); Event H7-1 (sugar beet, herbicide tolerance, deposited as NCIMB 41158 or NCIMB 41159, described in US-A 2004-172669 or WO 04/074492); Event JOPLINi (wheat, disease tolerance, not deposited, described in US-A 2008-064032); Event LL27 (soybean, herbicide tolerance, deposited as NCIMB41658, described in WO 06/108674 or US-A 2008-320616); Event LL55 (soybean, herbicide tolerance, deposited as NCIMB 41660, described in WO 06/108675 or US-A 2008-196127); Event LLcotton25 (cotton, herbicide tolerance, deposited as ATCC PTA-3343, described in WO 03/013224 or US-A 2003-097687); Event LLRICE06 (rice, herbicide tolerance, deposited as ATCC 203353, described in U.S. Pat. No. 6,468,747 or WO 00/026345); Event LLRice62 (rice, herbicide tolerance, deposited as ATCC 203352, described in WO 2000/026345), Event LLRICE601 (rice, herbicide tolerance, deposited as ATCC PTA-2600, described in US-A 2008-2289060 or WO 00/026356); Event LY038 (corn, quality trait, deposited as ATCC PTA-5623, described in US-A 2007-028322 or WO 05/061720); Event MIR162 (corn, insect control, deposited as PTA-8166, described in US-A 2009-300784 or WO 07/142840); Event MIR604 (corn, insect control, not deposited, described in US-A 2008-167456 or WO 05/103301); Event MON15985 (cotton, insect control, deposited as ATCC PTA-2516, described in US-A 2004-250317 or WO 02/100163); Event MON810 (corn, insect control, not deposited, described in US-A 2002-102582); Event MON863 (corn, insect control, deposited as ATCC PTA-2605, described in WO 04/011601 or US-A 2006-095986); Event MON87427 (corn, pollination control, deposited as ATCC PTA-7899, described in WO 11/062904); Event MON87460 (corn, stress tolerance, deposited as ATCC PTA-8910, described in WO 09/111263 or as US-A 2011-0138504); Event MON87701 (soybean, insect control, deposited as ATCC PTA-8194, described in US-A 2009-130071 or WO 09/064652); Event MON87705 (soybean, quality trait-herbicide tolerance, deposited as ATCC PTA-9241, described in US-A 2010-0080887 or WO 10/037016); Event MON87708 (soybean, herbicide tolerance, deposited as ATCC PTA-9670, described in WO 11/034704); Event MON87712 (soybean, yield, deposited as PTA-10296, described in WO 2012/051199), Event MON87754 (soybean, quality trait, deposited as ATCC PTA-9385, described in WO 10/024976); Event MON87769 (soybean, quality trait, deposited as ATCC PTA-8911, described in US-A 2011-0067141 or WO 09/102873); Event MON88017 (corn, insect control-herbicide tolerance, deposited as ATCC PTA-5582, described in US-A 2008-028482 or WO 05/059103); Event MON88913 (cotton, herbicide tolerance, deposited as ATCC PTA-4854, described in WO 04/072235 or US-A 2006-059590); Event MON88302 (oilseed rape, herbicide tolerance, deposited as PTA-10955, described in WO 2011/153186), Event MON88701 (cotton, herbicide tolerance, deposited as PTA-11754, described in WO 2012/134808), Event MON89034 (corn, insect control, deposited as ATCC PTA-7455, described in WO 07/140256 or US-A 2008-260932); Event MON89788 (soybean, herbicide tolerance, deposited as ATCC PTA-6708, described in US-A 2006-282915 or WO 06/130436); Event MS11 (oilseed rape, pollination control-herbicide tolerance, deposited as ATCC PTA-850 or PTA-2485, described in WO 01/031042); Event MS8 (oilseed rape, pollination control-herbicide tolerance, deposited as ATCC PTA-730, described in WO 01/041558 or US-A 2003-188347); Event NK603 (corn, herbicide tolerance, deposited as ATCC PTA-2478, described in US-A 2007-292854); Event PE-7 (rice, insect control, not deposited, described in WO 08/114282); Event RF3 (oilseed rape, pollination control-herbicide tolerance, deposited as ATCC PTA-730, described in WO 01/041558 or US-A 2003-188347); Event RT73 (oilseed rape, herbicide tolerance, not deposited, described in WO 02/036831 or US-A 2008-070260); Event SYHTOH2/SYN-000H2-5 (soybean, herbicide tolerance, deposited as PTA-11226, described in WO 2012/082548), Event T227-1 (sugar beet, herbicide tolerance, not deposited, described in WO 02/44407 or US-A 2009-265817); Event T25 (corn, herbicide tolerance, not deposited, described in US-A 2001-029014 or WO 01/051654); Event T304-40 (cotton, insect control-herbicide tolerance, deposited as ATCC PTA-8171, described in US-A 2010-077501 or WO 08/122406); Event T342-142 (cotton, insect control, not deposited, described in WO 06/128568); Event TC1507 (corn, insect control-herbicide tolerance, not deposited, described in US-A 2005-039226 or WO 04/099447); Event VIP1034 (corn, insect control-herbicide tolerance, deposited as ATCC PTA-3925, described in WO 03/052073), Event 32316 (corn, insect control-herbicide tolerance, deposited as PTA-11507, described in WO 11/084632), Event 4114 (corn, insect control-herbicide tolerance, deposited as PTA-11506, described in WO 11/084621), event EE-GM3/FG72 (soybean, herbicide tolerance, ATCC Accession No PTA-11041, WO 2011/063413A2), event DAS-68416-4 (soybean, herbicide tolerance, ATCC Accession No PTA-10442, WO2011/066360A1), event DAS-68416-4 (soybean, herbicide tolerance, ATCC Accession No PTA-10442, WO 2011/066384A1), event DP-040416-8 (corn, insect control, ATCC Accession No PTA-11508, WO 2011/075593A1), event DP-043A47-3 (corn, insect control, ATCC Accession No PTA-11509, WO 2011/075595A1), event DP-004114-3 (corn, insect control, ATCC Accession No PTA-11506, WO 2011/084621A1), event DP-032316-8 (corn, insect control, ATCC Accession No PTA-11507, WO 2011/084632A1), event MON-88302-9 (oilseed rape, herbicide tolerance, ATCC Accession No PTA-10955, WO 2011/153186A1), event DAS-21606-3 (soybean, herbicide tolerance, ATCC Accession No. PTA-11028, WO 2012/033794A2), event MON-87712-4 (soybean, quality trait, ATCC Accession No. PTA-10296, WO 2012/051199A2), event DAS-44406-6 (soybean, stacked herbicide tolerance, ATCC Accession No. PTA-11336, WO 2012/075426A1), event DAS-14536-7 (soybean, stacked herbicide tolerance, ATCC Accession No. PTA-11335, WO 2012/075429A1), event SYN-000H2-5 (soybean, herbicide tolerance, ATCC Accession No. PTA-11226, WO 2012/082548A2), event DP-061061-7 (oilseed rape, herbicide tolerance, no deposit No available, WO 2012071039A1), event DP-0734964 (oilseed rape, herbicide tolerance, no deposit No available, US2012131692), event 8264.44.06.1 (soybean, stacked herbicide tolerance, Accession No PTA-11336, WO 2012075426A2), event 8291.45.36.2 (soybean, stacked herbicide tolerance, Accession No. PTA-11335, WO 2012075429A2), event SYHT0H2 (soybean, ATCC Accession No. PTA-11226, WO 2012/082548A2), event MON88701 (cotton, ATCC Accession No PTA-11754, WO 2012/134808A1), event KK179-2 (alfalfa, ATCC Accession No PTA-11833, WO2013003558A1), event pDAB8264.42.32.1 (soybean, stacked herbicide tolerance, ATCC Accession No PTA-11993, WO 2013010094A1), event MZDT09Y (corn, ATCC Accession No PTA-13025, WO 2013012775A1).

The treatment of the plants and plant parts with the compounds of formula (I) is carried out directly or by acting on the environment, habitat or storage space using customary treatment methods, for example by dipping, spraying, atomizing, misting, evaporating, dusting, fogging, scattering, foaming, painting on, spreading, injecting, drenching, trickle irrigation and, in the case of propagation material, in particular in the case of seed, furthermore by the dry seed treatment method, the wet seed treatment method, the slurry treatment method, by encrusting, by coating with one or more coats and the like. It is furthermore possible to apply the active substances by the ultra-low volume method or to inject the active substance preparation or the active substance itself into the soil.

A preferred direct treatment of the plants is the leaf application treatment, i.e. compounds of formula (I) or compositions containing them are applied to the foliage, it being possible for the treatment frequency and the application rate to be matched to the infection pressure.

In the case of systemically active compounds, compounds of formula (I) or compositions according to the so invention reach the plants via the root system. In this case, the treatment of the plants is effected by allowing the compounds of formula (I) or compositions according to the invention to act on the environment of the plant. This can be done for example by drenching, incorporating in the soil or into the nutrient solution, i.e. the location of the plant (for example the soil or hydroponic systems) is impregnated with a liquid form of compounds of formula (I) or compositions according to the invention, or by soil application, i.e. the compounds of formula (I) or compositions according to the invention are incorporated into the location of the plants in solid form (for example in the form of granules). In the case of paddy rice cultures, this may also be done by metering the compounds of formula (I) or compositions according to the invention into a flooded paddy field in a solid use form (for example in the form of granules).

### PREPARATION EXAMPLES

The various aspects of the invention will now be illustrated with reference to the following Table 1 of compound examples and the following preparation or efficacy examples.

Table 1 illustrates in a non limiting manner examples of compounds of formula (I)

according to the invention. Each of those compounds represents a preferred embodiment of the invention.

TABLE 1

| | | | | | | | | | | | | Specific | |
|---|---|---|---|---|---|---|---|---|---|---|---|---|---|
| Example | | | | | | | | | | | | stereochemistry | |
| number | A | B | T | X1 | X2 | X3 | X4 | X5 | Z1 | Z2 | Z3 | C1(Z²)-C2(Z¹) | logP |
| 1 | 1-methyl-3-(trifluoromethyl)-1H-pyrazol-4-yl | cyclohexyl | O | H | H | H | H | H | H | H | H | | |
| 2 | 2-methyl-4,5-dihydrofuran-3-yl | cyclohexyl | O | H | H | H | H | H | H | H | H | | |
| 3 | 5-fluoro-1,3-dimethyl-1H-pyrazol-4-yl | cyclohexyl | O | H | H | H | H | H | H | H | H | | |
| 4 | 1-methyl-3-(trifluoromethyl)-1H-pyrazol-4-yl | cyclopropyl | O | H | H | H | H | H | H | H | H | | 2.34^{[b]} |
| 5 | 5-fluoro-1,3-dimethyl-1H-pyrazol-4-yl | cyclopropyl | O | H | H | H | H | H | H | H | H | | 2.00^{[b]} |
| 6 | 1,3-dimethyl-1H-pyrazol-4-yl | cyclopropyl | O | H | H | H | H | H | H | H | H | | 1.70^{[b]} |
| 7 | 1,3,5-trimethyl-1H-pyrazol-4-yl | cyclopropyl | O | H | H | H | H | H | H | H | H | | |
| 8 | 2-methyl-4-(trifluoromethyl)-1,3-thiazol-5-yl | cyclopropyl | O | H | H | H | H | H | H | H | H | | 2.66^{[b]} |
| 9 | 5-chloro-1,3-dimethyl-1H-pyrazol-4-yl | cyclopropyl | O | H | H | H | H | H | H | H | H | | 2.12^{[b]} |
| 10 | 2-methyl-4-(trifluoromethyl)-1,3-thiazol-5-yl | cyclopropyl | O | H | H | bromo | H | H | H | H | H | | |
| 11 | 1-methyl-3-(trifluoromethyl)-1H-pyrazol-4-yl | cyclopropyl | O | H | H | chloro | H | H | H | H | H | | 3.43^{[b]} |
| 12 | 5-fluoro-1,3-dimethyl-1H-pyrazol-4-yl | cyclopropyl | O | H | H | chloro | H | H | H | H | H | | 3.04^{[b]} |
| 13 | 1,3,5-trimethyl-1H-pyrazol-4-yl | cyclopropyl | O | H | H | chloro | H | H | H | H | H | | 2.89^{[b]} |
| 14 | 5-chloro-1,3-dimethyl-1H-pyrazol-4-yl | cyclopropyl | O | H | H | chloro | H | H | H | H | H | | 2.22^{[b]} |
| 15 | 1,3,5-trimethyl-1H-pyrazol-4-yl | cyclopropyl | O | H | H | fluoro | H | H | H | H | H | | 2.84^{[b]} |
| 16 | 1,3-dimethyl-1H-pyrazol-4-yl | cyclopropyl | O | H | H | fluoro | H | H | H | H | H | | 2.13^{[b]} |
| 17 | 1-methyl-3-(trifluoromethyl)-1H-pyrazol-4-yl | cyclopropyl | O | H | H | fluoro | H | H | H | H | H | | |
| 18 | 5-fluoro-1,3-dimethyl-1H-pyrazol-4-yl | cyclopropyl | O | H | H | fluoro | H | H | H | H | H | | 3.01^{[b]} |
| 19 | 1,3-dimethyl-1H-pyrazol-4-yl | cyclopropyl | O | H | H | chloro | H | H | H | H | H | | 2.63^{[b]} |
| 20 | 5-chloro-1,3-dimethyl-1H-pyrazol-4-yl | cyclopropyl | O | H | H | fluoro | H | H | H | H | H | | |
| 21 | 1,3,5-trimethyl-1H-pyrazol-4-yl | cyclopropyl | O | H | H | bromo | H | H | H | H | H | | 3.26^{[b]} |
| 22 | 5-fluoro-1,3-dimethyl-1H-pyrazol-4-yl | cyclopropyl | O | H | H | bromo | H | H | H | H | H | | |
| 23 | 1,3-dimethyl-1H-pyrazol-4-yl | cyclopropyl | O | H | H | bromo | H | H | H | H | H | | 3.20^{[b]} |
| 24 | 5-chloro-1,3-dimethyl-1H-pyrazol-4-yl | cyclopropyl | O | H | H | bromo | H | H | H | H | H | | |
| 25 | 1-methyl-3-(trifluoromethyl)-1H-pyrazol-4-yl | cyclopropyl | O | H | H | bromo | H | H | H | H | H | | 3.23^{[b]} |
| 26 | 2-methyl-4-(trifluoromethyl)-1,3-thiazol-5-yl | cyclopropyl | O | H | H | fluoro | H | H | H | H | H | | 3.87^{[b]} |
| 27 | 6-chloropyridin-3-yl | cyclopropyl | O | H | H | H | H | H | H | H | H | | |
| 28 | 2-methyl-4-(trifluoromethyl)-1,3-thiazol-5-yl | cyclopropyl | O | H | H | chloro | H | H | H | H | H | | 4.35^{[b]} |
| 29 | 6-chloropyridin-3-yl | cyclopropyl | O | H | H | fluoro | H | H | H | H | H | | |
| 30 | 6-chloropyridin-3-yl | cyclopropyl | O | H | H | bromo | H | H | H | H | H | | |
| 31 | 6-chloropyridin-3-yl | cyclopropyl | O | H | H | chloro | H | H | H | H | H | | 3.53^{[b]} |
| 32 | 2-chloropyridin-3-yl | cyclopropyl | O | H | H | H | H | H | H | H | H | | |
| 33 | 2-chloropyridin-3-yl | cyclopropyl | O | H | H | fluoro | H | H | H | H | H | | |
| 34 | 2-chloropyridin-3-yl | cyclopropyl | O | H | H | chloro | H | H | H | H | H | | 2.39^{[c]}; |
| | | | | | | | | | | | | | 2.43^{[b]} |
| 35 | 2-chloropyridin-3-yl | cyclopropyl | O | H | H | bromo | H | H | H | H | H | | 2.34^{[b]} |
| 36 | 4,6-dichloropyridin-3-yl | cyclopropyl | O | H | H | fluoro | H | H | H | H | H | | 2.70^{[b]} |
| 37 | 4,5-dibromo-2-thienyl | cyclopropyl | O | H | H | H | H | H | H | H | H | rel-(1R,2S) | |
| 38 | 7-bromo-1-benzothiophen-2-yl | cyclopropyl | O | H | H | H | H | H | H | H | H | rel-(1R,2S) | |
| 39 | pyridin-4-yl | cyclopropyl | O | H | H | H | H | H | H | H | H | | 1.30^{[b]} |
| 40 | 2-(trifluoromethyl)phenyl | cyclopropyl | O | H | H | H | H | H | H | H | H | | 2.79^{[b]} |
| 41 | 3-methoxy-1-methyl-1H-pyrazol-4-yl | cyclopropyl | O | H | H | H | H | H | H | H | H | rel-(1R,2S) | 2.03^{[b]} |
| 42 | 5-fluoro-1,3-dimethyl-1H-pyrazol-4-yl | cyclopropyl | O | H | H | H | H | H | H | H | H | rel-(1R,2S) | 1.99^{[b]} |
| 43 | 3-ethoxy-1-methyl-1H-pyrazol-4-yl | cyclopropyl | O | H | H | H | H | H | H | H | H | rel-(1R,2S) | 2.36^{[b]} |
| 44 | 2-iodophenyl | cyclopropyl | O | H | H | H | H | H | H | H | H | rel-(1R,2S) | 2.93^{[b]} |
| 45 | 2-bromophenyl | cyclopropyl | O | H | H | H | H | H | H | H | H | rel-(1R,2S) | 2.79^{[b]} |
| 46 | 2-chlorophenyl | cyclopropyl | O | H | H | H | H | H | H | H | H | rel-(1R,2S) | 2.68^{[b]} |
| 47 | 2,6-dichlorophenyl | cyclopropyl | O | H | H | H | H | H | H | H | H | rel-(1R,2S) | 2.99^{[b]} |
| 48 | 2-bromophenyl | cyclopropyl | O | H | H | H | H | chloro | H | H | H | | 3.19^{[b]} |
| 49 | 5-fluoro-1,3-dimethyl-1H-pyrazol-4-yl | cyclopropyl | O | H | H | H | H | chloro | H | H | H | rel-(1R,2S) | 2.40^{[b]} |
| 50 | 2,6-dichlorophenyl | cyclopropyl | O | H | H | H | H | chloro | H | H | H | rel-(1R,2S) | 3.32^{[b]} |
| 51 | 2-(trifluoromethyl)phenyl | cyclopropyl | O | H | H | H | H | chloro | H | H | H | rel-(1R,2S) | 3.31^{[b]} |
| 52 | 3-(difluoromethyl)-1-methyl-1H-pyrazol-4-yl | cyclopentyl | O | H | H | fluoro | fluoro | H | H | H | H | rel-(1R,2R) | 2.64^{[b]} |
| 53 | 3-(1-fluoroethyl)-1-methyl-1H-pyrazol-4-yl | cyclopentyl | O | H | H | fluoro | fluoro | H | H | H | H | rel-(1R,2R) | 2.62^{[b]} |
| 54 | 3-ethoxy-1-methyl-1H-pyrazol-4-yl | cyclopentyl | O | H | H | fluoro | fluoro | H | H | H | H | rel-(1R,2R) | 2.78^{[b]} |
| 55 | 2-methyl-4-(trifluoromethyl)-1,3-thiazol-5-yl | cyclopentyl | O | H | H | fluoro | fluoro | H | H | H | H | rel-(1R,2R) | 3.19^{[b]} |
| 56 | 2-iodo-3-thienyl | cyclopentyl | O | H | H | fluoro | fluoro | H | H | H | H | rel-(1R,2R) | 3.29^{[b]} |
| 57 | 2-(trifluoromethyl)-5,6-dihydro-1,4-oxathiin-3-yl | cyclopentyl | O | H | H | fluoro | fluoro | H | H | H | H | rel-(1R,2R) | 3.19^{[b]} |
| 58 | 2-iodophenyl | cyclopentyl | O | H | H | fluoro | fluoro | H | H | H | H | rel-(1R,2R) | 3.31^{[b]} |
| 59 | 2-methyl-3-furyl | cyclopentyl | O | H | H | fluoro | fluoro | H | H | H | H | rel-(1R,2R) | 3.06^{[b]} |
| 60 | 3-methyl-1,2-oxazol-4-yl | cyclopentyl | O | H | H | fluoro | fluoro | H | H | H | H | rel-(1R,2S) | 2.78^{[b]} |
| 61 | 3-methyl-1,2-oxazol-4-yl | cyclopentyl | O | H | H | fluoro | fluoro | H | H | H | H | rel-(1R,2R) | 2.69^{[b]} |
| 62 | 2-methylpyridin-3-yl | cyclopentyl | O | H | H | fluoro | fluoro | H | H | H | H | rel-(1R,2R) | 1.67^{[b]} |
| 63 | 2-methylpyridin-3-yl | cyclopentyl | O | H | H | fluoro | fluoro | H | H | H | H | rel-(1R,2S) | 1.75^{[b]} |
| 64 | 3-methylpyridin-2-yl | cyclopentyl | O | H | H | fluoro | fluoro | H | H | H | H | rel-(1R,2R) | 3.31^{[b]} |
| 65 | 4-methylpyrimidin-5-yl | cyclopentyl | O | H | H | fluoro | fluoro | H | H | H | H | rel-(1R,2R) | 2.15^{[b]} |
| 66 | 2-fluorophenyl | cyclopentyl | O | H | H | fluoro | fluoro | H | H | H | H | rel-(1R,2R) | 3.33^{[b]} |
| 67 | 2,5-dimethyl-3-furyl | cyclopentyl | O | H | H | fluoro | fluoro | H | H | H | H | rel-(1R,2R) | 3.35^{[b]} |
| 68 | 2-fluoropyridin-3-yl | cyclopentyl | O | H | H | fluoro | fluoro | H | H | H | H | rel-(1R,2R) | 2.76^{[b]} |
| 69 | 3-fluoropyridin-4-yl | cyclopentyl | O | H | H | fluoro | fluoro | H | H | H | H | rel-(1R,2R) | 2.59^{[b]} |
| 70 | 3-methyl-2-thienyl | cyclopentyl | O | H | H | fluoro | fluoro | H | H | H | H | rel-(1R,2R) | 3.39^{[b]} |
| 71 | 2-methyl-1,3-thiazol-5-yl | cyclopentyl | O | H | H | fluoro | fluoro | H | H | H | H | rel-(1R,2R) | 2.57^{[b]} |
| 72 | 4-methyl-1,2,3-thiadiazol-5-yl | cyclopentyl | O | H | H | fluoro | fluoro | H | H | H | H | rel-(1R,2R) | 2.86^{[b]} |
| 73 | 2-cyanophenyl | cyclopentyl | O | H | H | fluoro | fluoro | H | H | H | H | rel-(1R,2R) | 3.04^{[b]} |
| 74 | 2-cyanopyridin-3-yl | cyclopentyl | O | H | H | fluoro | fluoro | H | H | H | H | rel-(1R,2R) | 3.21^{[b]} |
| 75 | 2-methoxyphenyl | cyclopentyl | O | H | H | fluoro | fluoro | H | H | H | H | rel-(1R,2R) | 3.39^{[b]} |
| 76 | 3-methoxy-1-methyl-1H-pyrazol-4-yl | cyclopentyl | O | H | H | fluoro | fluoro | H | H | H | H | rel-(1R,2R) | 2.64^{[b]} |
| 77 | 5-fluoro-1,3-dimethyl-1H-pyrazol-4-yl | cyclopentyl | O | H | H | fluoro | fluoro | H | H | H | H | rel-(1R,2R) | 2.60^{[b]} |
| 78 | 4-methoxy-3-thienyl | cyclopentyl | O | H | H | fluoro | fluoro | H | H | H | H | rel-(1R,2R) | 3.29^{[b]} |
| 79 | 2-methyl-5,6-dihydro-1,4-oxathiin-3-yl | cyclopentyl | O | H | H | fluoro | fluoro | H | H | H | H | rel-(1R,2R) | 3.15^{[b]} |
| 80 | 3-chloro-2-thienyl | cyclopentyl | O | H | H | fluoro | fluoro | H | H | H | H | rel-(1R,2R) | 3.72^{[b]} |
| 81 | 1-methyl-3-propyl-1H-pyrazol-4-yl | cyclopentyl | O | H | H | fluoro | fluoro | H | H | H | H | rel-(1R,2R) | 2.80^{[b]} |
| 82 | 2-(methylsulfanyl)phenyl | cyclopentyl | O | H | H | fluoro | fluoro | H | H | H | H | rel-(1R,2R) | 3.35^{[b]} |
| 83 | 3-ethyl-5-fluoro-1-methyl-1H-pyrazol-4-yl | cyclopentyl | O | H | H | fluoro | fluoro | H | H | H | H | rel-(1R,2R) | 2.92^{[b]} |
| 84 | 1-ethyl-5-fluoro-3-methyl-1H-pyrazol-4-yl | cyclopentyl | O | H | H | fluoro | fluoro | H | H | H | H | rel-(1R,2R) | 2.92^{[b]} |
| 85 | 5-chloro-1,3-dimethyl-1H-pyrazol-4-yl | cyclopentyl | O | H | H | fluoro | fluoro | H | H | H | H | rel-(1R,2R) | 2.82^{[b]} |
| 86 | 2-propoxyphenyl | cyclopentyl | O | H | H | fluoro | fluoro | H | H | H | H | rel-(1R,2R) | 4.19^{[b]} |
| 87 | 5-fluoro-1-methyl-3-propyl-1H-pyrazol-4-yl | cyclopentyl | O | H | H | fluoro | fluoro | H | H | H | H | rel-(1R,2R) | 3.25^{[b]} |
| 88 | 2-(trifluoromethyl)phenyl | cyclopentyl | O | H | H | fluoro | fluoro | H | H | H | H | rel-(1R,2R) | 3.50^{[b]} |
| 89 | 4-(trifluoromethyl)pyridin-3-yl | cyclopentyl | O | H | H | fluoro | fluoro | H | H | H | H | rel-(1R,2R) | 2.92^{[b]} |
| 90 | 3-(trifluoromethyl)pyridin-2-yl | cyclopentyl | O | H | H | fluoro | fluoro | H | H | H | H | rel-(1R,2R) | 3.31^{[b]} |
| 91 | 1-methyl-4-(trifluoromethyl)-1H-pyrrol-3-yl | cyclopentyl | O | H | H | fluoro | fluoro | H | H | H | H | rel-(1R,2R) | 3.27^{[b]} |
| 92 | 1-methyl-3-(trifluoromethyl)-1H-pyrazol-4-yl | cyclopentyl | O | H | H | fluoro | fluoro | H | H | H | H | rel-(1R,2R) | 2.98^{[b]} |
| 93 | 3-(trifluoromethyl)-2-thienyl | cyclopentyl | O | H | H | fluoro | fluoro | H | H | H | H | rel-(1R,2R) | 3.63^{[b]} |
| 94 | 2-bromophenyl | cyclopentyl | O | H | H | fluoro | fluoro | H | H | H | H | rel-(1R,2R) | 3.35^{[b]} |
| 95 | 2-(trifluoromethoxy)phenyl | cyclopentyl | O | H | H | fluoro | fluoro | H | H | H | H | rel-(1R,2R) | 3.76^{[b]} |
| 96 | 3-bromo-2-thienyl | cyclopentyl | O | H | H | fluoro | fluoro | H | H | H | H | rel-(1R,2R) | 3.79^{[b]} |
| 97 | 2-amino-4-(trifluoromethyl)-1,3-thiazol-5-yl | cyclopentyl | O | H | H | fluoro | fluoro | H | H | H | H | rel-(1R,2R) | 2.82^{[b]} |
| 98 | 2-benzylphenyl | cyclopentyl | O | H | H | fluoro | fluoro | H | H | H | H | rel-(1R,2R) | 4.26^{[b]} |
| 99 | 3-iodo-1-methyl-1H-pyrazol-4-yl | cyclopentyl | O | H | H | fluoro | fluoro | H | H | H | H | rel-(1R,2R) | 2.75^{[b]} |
| 100 | 3-iodo-1-methyl-1H-pyrrol-2-yl | cyclopentyl | O | H | H | fluoro | fluoro | H | H | H | H | rel-(1R,2R) | 3.83^{[b]} |
| 101 | 3-iodo-2-thienyl | cyclopentyl | O | H | H | fluoro | fluoro | H | H | H | H | rel-(1R,2R) | 3.79^{[b]} |
| 102 | 5-methyl-1,2-oxazol-4-yl | cyclopentyl | O | H | H | fluoro | fluoro | H | H | H | H | rel-(1R,2S) | 2.82^{[b]} |
| 103 | 3-methylpyridin-2-yl | cyclopentyl | O | H | H | fluoro | fluoro | H | H | H | H | rel-(1R,2S) | 3.33^{[b]} |
| 104 | 4-methylpyrimidin-5-yl | cyclopentyl | O | H | H | fluoro | fluoro | H | H | H | H | rel-(1R,2S) | 2.21^{[b]} |
| 105 | 2-fluorophenyl | cyclopentyl | O | H | H | fluoro | fluoro | H | H | H | H | rel-(1R,2S) | 3.35^{[b]} |
| 106 | 2-fluoropyridin-3-yl | cyclopentyl | O | H | H | fluoro | fluoro | H | H | H | H | rel-(1R,2S) | 2.80^{[b]} |
| 107 | 3-fluoropyridin-4-yl | cyclopentyl | O | H | H | fluoro | fluoro | H | H | H | H | rel-(1R,2S) | 2.62^{[b]} |
| 108 | 2-methyl-1,3-thiazol-5-yl | cyclopentyl | O | H | H | fluoro | fluoro | H | H | H | H | rel-(1R,2S) | 2.64^{[b]} |
| 109 | 4-methyl-1,2,3-thiadiazol-5-yl | cyclopentyl | O | H | H | fluoro | fluoro | H | H | H | H | rel-(1R,2S) | 2.92^{[b]} |
| 110 | 2-cyanopyridin-3-yl | cyclopentyl | O | H | H | fluoro | fluoro | H | H | H | H | rel-(1R,2S) | 3.31^{[b]} |
| 111 | 1-methyl-1H-pyrrol-2-yl | cyclopentyl | O | H | H | fluoro | fluoro | H | H | H | H | rel-(1R,2R) | 3.13^{[b]} |
| 112 | 5-methyl-1,2-oxazol-4-yl | cyclopentyl | O | H | H | fluoro | fluoro | H | H | H | H | rel-(1R,2R) | 2.73^{[b]} |
| 113 | 1-methyl-3-oxo-2,3-dihydro-1H-pyrazol-4-yl | cyclopentyl | O | H | H | fluoro | fluoro | H | H | H | H | rel-(1R,2R) | 2.00^{[b]} |
| 114 | 2-ethylphenyl | cyclopentyl | O | H | H | fluoro | fluoro | H | H | H | H | rel-(1R,2R) | 3.75^{[b]} |
| 115 | 2-ethoxyphenyl | cyclopentyl | O | H | H | fluoro | fluoro | H | H | H | H | rel-(1R,2R) | 3.80^{[b]} |
| 116 | 5-methoxy-1,3-dimethyl-1H-pyrazol-4-yl | cyclopentyl | O | H | H | fluoro | fluoro | H | H | H | H | rel-(1R,2R) | 2.59^{[b]} |
| 117 | 2-chloro-6-methylpyridin-3-yl | cyclopentyl | O | H | H | fluoro | fluoro | H | H | H | H | rel-(1R,2R) | 2.86^{[b]} |
| 118 | 3-isopropoxy-1-methyl-1H-pyrazol-4-yl | cyclopentyl | O | H | H | fluoro | fluoro | H | H | H | H | rel-(1R,2R) | 3.27^{[b]} |
| 119 | 2-(difluoromethoxy)phenyl | cyclopentyl | O | H | H | fluoro | fluoro | H | H | H | H | rel-(1R,2R) | 3.51^{[b]} |
| 120 | 2,4-dichloropyridin-3-yl | cyclopentyl | O | H | H | fluoro | fluoro | H | H | H | H | rel-(1R,2R) | 3.02^{[b]} |
| 121 | 3,4-dichloro-1,2-thiazol-5-yl | cyclopentyl | O | H | H | fluoro | fluoro | H | H | H | H | rel-(1R,2R) | 4.19^{[b]} |
| 122 | 5-fluoro-1-methyl-3-(trifluoromethyl)-1H-pyrazol-4-yl | cyclopentyl | O | H | H | fluoro | fluoro | H | H | H | H | rel-(1R,2R) | 3.27^{[b]} |
| 123 | 2-bromo-4-methyl-1,3-thiazol-5-yl | cyclopentyl | O | H | H | fluoro | fluoro | H | H | H | H | rel-(1R,2R) | 3.46^{[b]} |
| 124 | 2-chloro-6-(trifluoromethyl)pyridin-3-yl | cyclopentyl | O | H | H | fluoro | fluoro | H | H | H | H | rel-(1R,2R) | 3.68^{[b]} |
| 125 | 3-(difluoromethyl)-1-methyl-1H-pyrazol-4-yl | cyclopentyl | O | H | H | fluoro | fluoro | H | H | H | H | rel-(1R,2S) | 2.88^{[b]} |
| 126 | 3-(1-fluoroethyl)-1-methyl-1H-pyrazol-4-yl | cyclopentyl | O | H | H | fluoro | fluoro | H | H | H | H | rel-(1R,2S) | 2.84^{[b]} |
| 127 | 3-ethoxy-1-methyl-1H-pyrazol-4-yl | cyclopentyl | O | H | H | fluoro | fluoro | H | H | H | H | rel-(1R,2S) | 3.13^{[b]} |
| 128 | 2-methyl-4-(trifluoromethyl)-1,3-thiazol-5-yl | cyclopentyl | O | H | H | fluoro | fluoro | H | H | H | H | rel-(1R,2S) | 3.42^{[b]} |
| 129 | 2-iodo-3-thienyl | cyclopentyl | O | H | H | fluoro | fluoro | H | H | H | H | rel-(1R,2S) | 3.58^{[b]} |
| 130 | 2-(trifluoromethyl)-5,6-dihydro-1,4-oxathiin-3-yl | cyclopentyl | O | H | H | fluoro | fluoro | H | H | H | H | rel-(1R,2S) | 3.39^{[b]} |
| 131 | 2-iodophenyl | cyclopentyl | O | H | H | fluoro | fluoro | H | H | H | H | rel-(1R,2S) | 3.56^{[b]} |
| 132 | 1-methyl-1H-pyrrol-2-yl | cyclopentyl | O | H | H | fluoro | fluoro | H | H | H | H | rel-(1R,2S) | 3.23^{[b]} |
| 133 | 2-methyl-3-furyl | cyclopentyl | O | H | H | fluoro | fluoro | H | H | H | H | rel-(1R,2S) | 3.17^{[b]} |
| 134 | 2,5-dimethyl-3-furyl | cyclopentyl | O | H | H | fluoro | fluoro | H | H | H | H | rel-(1R,2S) | 3.46^{[b]} |
| 135 | 1-methyl-3-oxo-2,3-dihydro-1H-pyrazol-4-yl | cyclopentyl | O | H | H | fluoro | fluoro | H | H | H | H | rel-(1R,2S) | 2.18^{[b]} |
| 136 | 3-methyl-2-thienyl | cyclopentyl | O | H | H | fluoro | fluoro | H | H | H | H | rel-(1R,2S) | 3.46^{[b]} |
| 137 | 2-cyanophenyl | cyclopentyl | O | H | H | fluoro | fluoro | H | H | H | H | rel-(1R,2S) | 2.96^{[b]} |
| 138 | 3-(difluoromethyl)-1-methyl-1H-pyrazol-4-yl | cyclohexyl | O | H | H | H | H | H | H | H | cyclopropyl | rel-(1R,2R) | 3.60^{[b]} |
| 139 | 5-fluoro-1,3-dimethyl-1H-pyrazol-4-yl | cyclohexyl | O | H | H | H | H | H | H | H | cyclopropyl | rel-(1R,2R) | 3.53^{[b]} |
| 140 | 1-methyl-4-(trifluoromethyl)-1H-pyrrol-3-yl | cyclohexyl | O | H | H | H | H | H | H | H | cyclopropyl | rel-(1R,2S) | 3.90^{[b]} |
| 141 | 4-(difluoromethyl)-2-methyl-1,3-thiazol-5-yl | cyclohexyl | O | H | H | H | H | H | H | H | cyclopropyl | rel-(1R,2S) | 4.18^{[b]} |
| 142 | 1-methyl-3-(trifluoromethyl)-1H-pyrazol-4-yl | cyclohexyl | O | H | H | H | H | H | H | H | cyclopropyl | rel-(1R,2S) | 3.96^{[b]} |
| 143 | 3-(difluoromethyl)-1-methyl-1H-pyrazol-4-yl | cyclohexyl | O | H | H | H | H | H | H | H | cyclopropyl | rel-(1R,2S) | 3.55^{[b]} |
| 144 | 3-ethyl-5-fluoro-1-methyl-1H-pyrazol-4-yl | cyclohexyl | O | H | H | H | H | H | H | H | cyclopropyl | rel-(1R,2S) | 3.83^{[b]} |
| 145 | 3-iodo-1-methyl-1H-pyrazol-4-yl | cyclohexyl | O | H | H | H | H | H | H | H | cyclopropyl | rel-(1R,2S) | 3.60^{[b]} |
| 146 | 1,3,5-trimethyl-1H-pyrazol-4-yl | cyclohexyl | O | H | H | H | H | H | H | H | cyclopropyl | rel-(1R,2S) | 3.13^{[b]} |
| 147 | 2-methyl-4-(trifluoromethyl)-1,3-thiazol-5-yl | cyclohexyl | O | H | H | H | H | H | H | H | cyclopropyl | rel-(1R,2S) | 4.69^{[b]} |
| 148 | 2-(trifluoromethyl)phenyl | cyclopropyl | S | H | H | H | H | H | H | H | H | | 3.69^{[b]} |
| 149 | 3-(trifluoromethyl)-2-thienyl | cyclopentyl | S | H | H | fluoro | fluoro | H | H | H | H | | 4.59^{[b]} |
| 150 | 2-(trifluoromethyl)-5,6-dihydro-1,4-oxathiin-3-yl | cyclopentyl | S | H | H | fluoro | fluoro | H | H | H | H | | 4.21^{[b]} |
| 151 | 3-(difluoromethyl)-1-methyl-1H-pyrazol-4-yl | cyclopropyl | O | H | H | chloro | H | H | H | H | H | rel-(1R,2R) | 2.30^{[b]} |
| 152 | 1-methyl-4-(trifluoromethyl)-1H-pyrrol-3-yl | cyclohexyl | S | H | H | H | H | H | H | H | cyclopropyl | rel-(1R,2S) | |
| 153 | 5-fluoro-1,3-dimethyl-1H-pyrazol-4-yl | cyclohexyl | S | H | H | H | H | H | H | H | cyclopropyl | rel-(1R,2R) | |
| 154 | 5-fluoro-1,3-dimethyl-1H-pyrazol-4-yl | cyclohexyl | O | chloro | H | H | H | H | H | H | cyclopropyl | | 3.94^{[b]} |
| 155 | 3-(difluoromethyl)-1-methyl-1H-pyrazol-4-yl | cyclopentyl | O | chloro | H | chloro | H | H | H | H | cyclopropyl | | 4.23^{[b]} |
| 156 | 3-(difluoromethyl)-1-methyl-1H-pyrazol-4-yl | cyclopentyl | O | chloro | H | H | H | H | H | H | cyclopropyl | | 3.85^{[b]} |
| 157 | 3-(difluoromethyl)-1-methyl-1H-pyrazol-4-yl | cyclohexyl | O | chloro | H | chloro | H | H | H | H | cyclopropyl | | 4.59^{[b]} |
| 158 | 3-(difluoromethyl)-1-methyl-1H-pyrazol-4-yl | cyclohexyl | O | chloro | H | H | H | H | H | H | cyclopropyl | | 4.03^{[b]} |
| 159 | 5-fluoro-1,3-dimethyl-1H-pyrazol-4-yl | cyclopentyl | O | chloro | H | chloro | H | H | H | H | cyclopropyl | | 4.18^{[b]} |
| 160 | 3-ethyl-5-fluoro-1-methyl-1H-pyrazol-4-yl | cyclopentyl | O | chloro | H | chloro | H | H | H | H | cyclopropyl | | 4.65^{[b]} |
| 161 | 3-ethyl-5-fluoro-1-methyl-1H-pyrazol-4-yl | cyclohexyl | O | chloro | H | chloro | H | H | H | H | cyclopropyl | | 5.08^{[b]} |
| 162 | 3-ethyl-5-fluoro-1-methyl-1H-pyrazol-4-yl | cyclohexyl | O | chloro | H | H | H | H | H | H | cyclopropyl | | 4.39^{[b]} |
| 163 | 2,5-dimethyl-3-furyl | cyclohexyl | O | chloro | H | chloro | H | H | H | H | cyclopropyl | | 5.85^{[b]} |
| 164 | 5-fluoro-1,3-dimethyl-1H-pyrazol-4-yl | cyclopentyl | O | chloro | H | H | H | H | H | H | cyclopropyl | | 3.71^{[b]} |
| 165 | 5-fluoro-1,3-dimethyl-1H-pyrazol-4-yl | cyclohexyl | O | chloro | H | chloro | H | H | H | H | cyclopropyl | | 4.59^{[b]} |
| 166 | 3-(difluoromethyl)-5-fluoro-1-methyl-1H-pyrazol-4-yl | cyclopentyl | O | chloro | H | chloro | H | H | H | H | cyclopropyl | | 4.67^{[b]} |
| 167 | 3-(difluoromethyl)-5-fluoro-1-methyl-1H-pyrazol-4-yl | cyclopentyl | O | chloro | H | H | H | H | H | H | cyclopropyl | | 4.31^{[b]} |
| 168 | 3-(difluoromethyl)-5-fluoro-1-methyl-1H-pyrazol-4-yl | cyclohexyl | O | chloro | H | chloro | H | H | H | H | cyclopropyl | | 5.05^{[b]} |
| 169 | 3-(difluoromethyl)-5-fluoro-1-methyl-1H-pyrazol-4-yl | cyclohexyl | O | chloro | H | H | H | H | H | H | cyclopropyl | | 4.49^{[b]} |
| 170 | 4-(difluoromethyl)-2-methyl-1,3-thiazol-5-yl | cyclohexyl | O | chloro | H | chloro | H | H | H | H | cyclopropyl | | 5.25^{[b]} |
| 171 | 4-(difluoromethyl)-2-methyl-1,3-thiazol-5-yl | cyclohexyl | O | chloro | H | H | H | H | H | H | cyclopropyl | | 4.67^{[b]} |
| 172 | 3-ethyl-5-fluoro-1-methyl-1H-pyrazol-4-yl | cyclopentyl | O | chloro | H | H | H | H | H | H | cyclopropyl | | 4.21^{[b]} |
| 173 | 4-(difluoromethyl)-2-methyl-1,3-thiazol-5-yl | cyclopentyl | O | chloro | H | chloro | H | H | H | H | cyclopropyl | | 4.85^{[b]} |
| 174 | 4-(difluoromethyl)-2-methyl-1,3-thiazol-5-yl | cyclopentyl | O | chloro | H | H | H | H | H | H | cyclopropyl | | 4.51^{[b]} |
| 175 | 2,5-dimethyl-3-furyl | cyclopentyl | O | chloro | H | chloro | H | H | H | H | cyclopropyl | | 5.39^{[b]} |
| 176 | 2,5-dimethyl-3-furyl | cyclopentyl | O | chloro | H | H | H | H | H | H | cyclopropyl | | 4.95^{[b]} |
| 177 | 2,5-dimethyl-3-furyl | cyclohexyl | O | chloro | H | H | H | H | H | H | cyclopropyl | | 5.17^{[b]} |
| 178 | 5-fluoro-1-methyl-3-(trifluoromethyl)-1H-pyrazol-4-yl | cyclopropyl | O | H | H | H | H | H | H | H | H | | 2.63^{[b]} |
| 179 | 3-(difluoromethyl)-5-fluoro-1-methyl-1H-pyrazol-4-yl | cyclopropyl | O | H | H | chloro | H | H | H | H | H | rel-(1R,2R) | 2.61^{[b]} |
| 180 | 3-(difluoromethyl)-5-fluoro-1-methyl-1H-pyrazol-4-yl | cyclohexyl | O | H | H | H | H | H | methyl | H | cyclopropyl | | 4.18^{[b]} |
| 181 | 3-(difluoromethyl)-5-fluoro-1-methyl-1H-pyrazol-4-yl | cyclohexyl | O | H | H | H | H | H | H | H | H | rel-(1R,2S) | 3.13^{[b]} |
| 182 | 3-(difluoromethyl)-5-fluoro-1-methyl-1H-pyrazol-4-yl | cyclohexyl | O | H | H | H | H | H | H | H | H | rel-(1R,2R) | 3.29^{[b]} |
| 183 | 5-chloro-3-(difluoromethyl)-1-methyl-1H-pyrazol-4-yl | cyclohexyl | O | H | H | H | H | H | H | H | H | rel-(1R,2S) | 3.51^{[b]} |
| 184 | 5-chloro-3-(difluoromethyl)-1-methyl-1H-pyrazol-4-yl | cyclohexyl | O | H | H | H | H | H | H | H | H | rel-(1R,2R) | 3.33^{[b]} |
| 185 | 3-(difluoromethyl)-1-methyl-1H-pyrazol-4-yl | cyclohexyl | O | H | H | H | H | H | H | H | H | rel-(1R,2R) | 2.76^{[b]} |
| 186 | 3-(difluoromethyl)-1-methyl-1H-pyrazol-4-yl | cyclohexyl | O | H | H | H | H | H | H | H | H | rel-(1R,2S) | 3.00^{[b]} |
| 187 | 3-(difluoromethyl)-5-fluoro-1-methyl-1H-pyrazol-4-yl | cyclopropyl | O | H | H | H | fluoro | H | H | H | H | | 2.59^{[b]} |
| 188 | 3-(difluoromethyl)-5-fluoro-1-methyl-1H-pyrazol-4-yl | cyclopropyl | O | H | H | fluoro | H | H | H | H | H | | 2.59^{[b]} |
| 189 | 3-(difluoromethyl)-5-fluoro-1-methyl-1H-pyrazol-4-yl | cyclopropyl | O | H | H | H | H | trifluoromethyl | H | H | H | | 3.00^{[b]} |
| 190 | 3-(difluoromethyl)-5-fluoro-1-methyl-1H-pyrazol-4-yl | cyclohexyl | S | H | H | H | H | H | methyl | H | cyclopropyl | | 5.01^{[b]} |
| 191 | 5-chloro-3-(difluoromethyl)-1-methyl-1H-pyrazol-4-yl | cyclopropyl | O | H | H | H | fluoro | H | H | H | H | | 2.75^{[b]} |
| 192 | 5-chloro-3-(difluoromethyl)-1-methyl-1H-pyrazol-4-yl | cyclopropyl | O | H | H | fluoro | H | H | H | H | H | | 2.75^{[b]} |
| 193 | 5-chloro-3-(difluoromethyl)-1-methyl-1H-pyrazol-4-yl | cyclopropyl | O | H | H | H | H | trifluoromethyl | H | H | H | | 3.15^{[b]} |
| 194 | 5-chloro-3-(trifluoromethyl)pyridin-2-yl | cyclopropyl | O | H | H | chloro | H | chloro | H | H | H | | |
| 195 | 5-chloro-3-(trifluoromethyl)pyridin-2-yl | cyclopropyl | O | H | H | chloro | H | H | H | H | H | | |
| 196 | 3-(difluoromethyl)-5-fluoro-1-methyl-1H-pyrazol-4-yl | cyclohexyl | O | H | H | H | H | H | H | H | methyl | | 3.27^{[b]} |
| 197 | 5-chloro-3-(difluoromethyl)-1-methyl-1H-pyrazol-4-yl | cyclohexyl | O | H | H | H | H | H | H | H | methyl | | 3.63^{[b]} |
| 198 | 2-chlorophenyl | cyclopropyl | O | H | H | chloro | H | H | H | H | H | | 3.02^{[c]}; |
| | | | | | | | | | | | | | 3.09^{[b]} |
| 199 | 2-iodophenyl | cyclopropyl | O | H | H | chloro | H | H | H | H | H | | 3.19^{[c]}; |
| | | | | | | | | | | | | | 3.24^{[b]} |
| 200 | 2,6-difluorophenyl | cyclopropyl | O | H | H | chloro | H | H | H | H | H | | 2.89^{[c]}; |
| | | | | | | | | | | | | | 2.94^{[b]} |
| 201 | 2-(trifluoromethyl)phenyl | cyclopropyl | O | H | H | chloro | H | H | H | H | H | | 3.21^{[c]}; |
| | | | | | | | | | | | | | 3.27^{[b]} |
| 202 | 3-iodo-2-thienyl | cyclopropyl | O | H | H | chloro | H | H | H | H | H | | 3.50^{[c]}; |
| | | | | | | | | | | | | | 3.56^{[b]} |
| 203 | 3-chloro-2-thienyl | cyclopropyl | O | H | H | chloro | H | H | H | H | H | | 3.45^{[c]}; |
| | | | | | | | | | | | | | 3.51^{[b]} |
| 204 | 3-bromo-2-furyl | cyclopropyl | O | H | H | chloro | H | H | H | H | H | | 2.98^{[c]}; |
| | | | | | | | | | | | | | 3.04^{[b]} |
| 205 | 2-fluorophenyl | cyclopropyl | O | chloro | H | chloro | H | H | H | H | H | rel-(1R,2S) | 3.55^{[c]}; |
| | | | | | | | | | | | | | 3.66^{[b]} |
| 206 | 2-chlorophenyl | cyclopropyl | O | chloro | H | chloro | H | H | H | H | H | rel-(1R,2S) | 3.47^{[c]}; |
| | | | | | | | | | | | | | 3.57^{[b]} |
| 207 | 2-bromophenyl | cyclopropyl | O | chloro | H | chloro | H | H | H | H | H | rel-(1R,2S) | 3.52^{[c]}; |
| | | | | | | | | | | | | | 3.58^{[b]} |
| 208 | 2-iodophenyl | cyclopropyl | O | chloro | H | chloro | H | H | H | H | H | rel-(1R,2S) | 3.64^{[c]}; |
| | | | | | | | | | | | | | 3.74^{[b]} |
| 209 | 2,6-difluorophenyl | cyclopropyl | O | chloro | H | chloro | H | H | H | H | H | rel-(1R,2S) | 3.29^{[c]}; |
| | | | | | | | | | | | | | 3.36^{[b]} |
| 210 | 2-(trifluoromethyl)phenyl | cyclopropyl | O | chloro | H | chloro | H | H | H | H | H | rel-(1R,2S) | 3.62^{[c]}; |
| | | | | | | | | | | | | | 3.73^{[b]} |
| 211 | 2-chloropyridin-3-yl | cyclopropyl | O | chloro | H | chloro | H | H | H | H | H | rel-(1R,2S) | 2.77^{[c]}; |
| | | | | | | | | | | | | | 2.82^{[b]} |
| 212 | 3-iodo-2-thienyl | cyclopropyl | O | chloro | H | chloro | H | H | H | H | H | rel-(1R,2S) | 4.04^{[c]}; |
| | | | | | | | | | | | | | 4.10^{[b]} |
| 213 | 3-chloro-2-thienyl | cyclopropyl | O | chloro | H | chloro | H | H | H | H | H | rel-(1R,2S) | 4.02^{[c]}; |
| | | | | | | | | | | | | | 4.08^{[b]} |
| 214 | 3-bromo-2-furyl | cyclopropyl | O | chloro | H | chloro | H | H | H | H | H | rel-(1R,2S) | 3.44^{[c]}; |
| | | | | | | | | | | | | | 3.56^{[b]} |
| 215 | 3-chloropyridin-2-yl | cyclobutyl | O | H | H | H | H | trifluoromethyl | H | H | H | rel-(1R,2R) | 3.01^{[b]} |
| 216 | 2-bromophenyl | cyclobutyl | O | H | H | H | H | trifluoromethyl | H | H | H | rel-(1R,2R) | 3.32^{[b]} |
| 217 | 2,4-dichlorophenyl | cyclobutyl | O | H | H | H | H | trifluoromethyl | H | H | H | rel-(1R,2R) | 3.81^{[b]} |
| 218 | 2-hydroxypyridin-3-yl | cyclopropyl | O | H | H | H | H | H | H | H | H | | |
| 219 | 2-methoxypyridin-3-yl | cyclopropyl | O | H | H | H | H | H | H | H | H | | |
| 220 | 2-ethoxypyridin-3-yl | cyclopropyl | O | H | H | H | H | H | H | H | H | | |
| 221 | 4-(trifluoromethyl)pyridin-3-yl | cyclopropyl | O | H | H | H | H | H | H | H | H | | |
| 222 | 2-(difluoromethyl)phenyl | cyclopropyl | O | H | H | H | H | H | H | H | H | | |
| 223 | 2-chloro-4-(trifluoromethyl)pyridin-3-yl | cyclopropyl | O | H | H | H | H | H | H | H | H | | |
| 224 | 2-(2,2,2-trifluoroethyl)pyridin-3-yl | cyclopropyl | O | H | H | H | H | H | H | H | H | | |
| 225 | 2-(difluoromethyl)pyridin-3-yl | cyclopropyl | O | H | H | H | H | H | H | H | H | | |
| 226 | 2-chloro-6-(trifluoromethyl)pyridin-3-yl | cyclopropyl | O | H | H | H | H | H | H | H | H | | |
| 227 | 2-bromophenyl | cyclobutyl | O | fluoro | H | H | H | fluoro | H | H | H | rel-(1R,2R) | 2.97^{[b]} |
| 228 | 3-chloro-5-(trifluoromethyl)pyridin-2-yl | cyclobutyl | O | fluoro | H | H | H | fluoro | H | H | H | rel-(1R,2R) | 3.61^{[b]} |
| 229 | 2-iodophenyl | cyclopropyl | O | chloro | H | H | H | H | H | H | H | | 3.14^{[b]} |
| 230 | 2-(trifluoromethyl)phenyl | cyclopropyl | O | chloro | H | H | H | H | H | H | H | | 3.13^{[b]} |
| 231 | 3-(difluoromethyl)-1-methyl-1H-pyrazol-4-yl | cyclopropyl | O | chloro | H | H | H | H | H | H | H | | 2.45^{[b]} |
| 232 | 2-iodophenyl | cyclopropyl | O | trifluoromethyl | H | H | H | H | H | H | H | | 3.31^{[b]} |
| 233 | 2-(trifluoromethyl)phenyl | cyclopropyl | O | trifluoromethyl | H | H | H | H | H | H | H | | 3.29^{[b]} |
| 234 | 3-(difluoromethyl)-1-methyl-1H-pyrazol-4-yl | cyclopropyl | O | trifluoromethyl | H | H | H | H | H | H | H | | 2.64^{[b]} |
| 235 | 2-(trifluoromethyl)pyridin-3-yl | cyclobutyl | O | H | H | H | H | trifluoromethyl | H | H | H | rel-(1R,2R) | 2.94^{[b]} |
| 236 | 2-(trifluoromethyl)pyridin-3-yl | cyclobutyl | O | fluoro | H | H | H | fluoro | H | H | H | rel-(1R,2R) | 2.60^{[b]} |
| 237 | 3-chloro-5-(trifluoromethyl)pyridin-2-yl | cyclobutyl | O | H | H | H | H | chloro | H | H | H | rel-(1R,2S) | 3.96^{[b]} |
| 238 | 3-chloropyridin-2-yl | cyclobutyl | O | H | H | H | H | chloro | H | H | H | rel-(1R,2S) | 3.06^{[b]} |
| 239 | 2-chlorophenyl | cyclobutyl | O | H | H | H | H | chloro | H | H | H | rel-(1R,2S) | 3.30^{[b]} |
| 240 | 2,4-dichlorophenyl | cyclobutyl | O | H | H | H | H | chloro | H | H | H | rel-(1R,2S) | 3.84^{[b]} |
| 241 | 2-iodophenyl | cyclobutyl | O | H | H | H | H | chloro | H | H | H | rel-(1R,2S) | 3.46^{[b]} |
| 242 | 2-(trifluoromethyl)phenyl | cyclobutyl | O | H | H | H | H | chloro | H | H | H | rel-(1R,2S) | 3.45^{[b]} |
| 243 | 2,6-difluorophenyl | cyclobutyl | O | H | H | H | H | chloro | H | H | H | rel-(1R,2S) | 3.11^{[b]} |
| 244 | 2-bromophenyl | cyclobutyl | O | H | H | H | H | chloro | H | H | H | rel-(1R,2S) | 3.33^{[b]} |
| 245 | 2-(trifluoromethyl)pyridin-3-yl | cyclobutyl | O | H | H | H | H | chloro | H | H | H | rel-(1R,2S) | 2.89^{[b]} |
| 246 | 3-chloro-5-(trifluoromethyl)pyridin-2-yl | cyclobutyl | O | H | H | H | H | chloro | H | H | H | rel-(1R,2R) | 3.77^{[b]} |
| 247 | 3-chloropyridin-2-yl | cyclobutyl | O | H | H | H | H | chloro | H | H | H | rel-(1R,2R) | 2.79^{[b]} |
| 248 | 2-chlorophenyl | cyclobutyl | O | H | H | H | H | chloro | H | H | H | rel-(1R,2R) | 3.08^{[b]} |
| 249 | 2,4-dichlorophenyl | cyclobutyl | O | H | H | H | H | chloro | H | H | H | rel-(1R,2R) | 3.65^{[b]} |
| 250 | 2-iodophenyl | cyclobutyl | O | H | H | H | H | chloro | H | H | H | rel-(1R,2R) | 3.23^{[b]} |
| 251 | 2-(trifluoromethyl)phenyl | cyclobutyl | O | H | H | H | H | chloro | H | H | H | rel-(1R,2R) | 3.23^{[b]} |
| 252 | 2,6-difluorophenyl | cyclobutyl | O | H | H | H | H | chloro | H | H | H | rel-(1R,2R) | 2.86^{[b]} |
| 253 | 2-bromophenyl | cyclobutyl | O | H | H | H | H | chloro | H | H | H | rel-(1R,2R) | 3.18^{[b]} |
| 254 | 2-(trifluoromethyl)pyridin-3-yl | cyclobutyl | O | H | H | H | H | chloro | H | H | H | rel-(1R,2R) | 2.69^{[b]} |
| 255 | 3-(difluoromethyl)-1-methyl-1H-pyrazol-4-yl | cyclobutyl | O | H | H | H | H | chloro | H | H | H | rel-(1R,2R) | 2.47^{[b]} |
| 256 | 2-chlorophenyl | cyclobutyl | O | H | H | chloro | H | chloro | H | H | H | rel-(1R,2S) | 3.90^{[b]} |
| 257 | 3-chloropyridin-2-yl | cyclobutyl | O | H | H | chloro | H | chloro | H | H | H | rel-(1R,2S) | 3.69^{[b]} |
| 258 | 3-chloro-5-(trifluoromethyl)pyridin-2-yl | cyclobutyl | O | H | H | chloro | H | chloro | H | H | H | rel-(1R,2S) | 4.53^{[b]} |
| 259 | 3-chloro-5-(trifluoromethyl)pyridin-2-yl | cyclobutyl | O | H | H | chloro | H | chloro | H | H | H | rel-(1R,2R) | 4.23^{[b]} |
| 260 | 3-chloropyridin-2-yl | cyclobutyl | O | H | H | chloro | H | chloro | H | H | H | rel-(1R,2R) | 3.28^{[b]} |
| 261 | 2-chlorophenyl | cyclobutyl | O | H | H | chloro | H | chloro | H | H | H | rel-(1R,2R) | 3.45^{[b]} |
| 262 | 3-(difluoromethyl)-1-methyl-1H-pyrazol-4-yl | cyclobutyl | O | H | H | H | H | chloro | H | H | H | rel-(1R,2S) | 2.72^{[b]} |
| 263 | 3-(difluoromethyl)-1-methyl-1H-pyrazol-4-yl | cyclobutyl | O | trifluoromethyl | H | H | H | H | H | H | H | rel-(1R,2R) | 2.75^{[b]} |
| 264 | 2-iodophenyl | cyclobutyl | O | chloro | H | chloro | H | H | H | H | H | rel-(1R,2S) | 4.09^{[b]} |
| 265 | 2,4-dichlorophenyl | cyclobutyl | O | chloro | H | chloro | H | H | H | H | H | rel-(1R,2S) | 4.48^{[b]} |
| 266 | 2,4-dichlorophenyl | cyclobutyl | O | chloro | H | chloro | H | H | H | H | H | rel-(1R,2R) | 4.16^{[b]} |
| 267 | 2,6-difluorophenyl | cyclobutyl | O | H | H | H | H | trifluoromethyl | H | H | H | rel-(1R,2S) | 3.24^{[b]} |
| 268 | 2,6-difluorophenyl | cyclopropyl | O | H | H | chloro | H | H | H | H | H | rel-(1R,2R) | 2.59^{[c]}; |
| | | | | | | | | | | | | | 2.64^{[b]} |
| 269 | 2-fluorophenyl | cyclopropyl | O | H | H | chloro | H | H | H | H | H | rel-(1R,2R) | 2.75^{[c]}; |
| | | | | | | | | | | | | | 2.79^{[b]} |
| 270 | 2-fluoro-6-(trifluoromethyl)phenyl | cyclopropyl | O | H | H | chloro | H | H | H | H | H | rel-(1R,2R) | 3.03^{[c]}; |
| | | | | | | | | | | | | | 3.08^{[b]} |
| 271 | 2-(trifluoromethyl)phenyl | cyclopropyl | O | H | H | chloro | H | H | H | H | H | rel-(1R,2R) | 2.94^{[c]}; |
| | | | | | | | | | | | | | 2.98^{[b]} |
| 272 | 2-bromophenyl | cyclopropyl | O | H | H | chloro | H | H | H | H | H | rel-(1R,2R) | 2.76^{[c]}; |
| | | | | | | | | | | | | | 2.82^{[b]} |
| 273 | 2-iodophenyl | cyclopropyl | O | H | H | chloro | H | H | H | H | H | rel-(1R,2R) | 2.88^{[c]}; |
| | | | | | | | | | | | | | 2.94^{[b]} |
| 274 | 2-chloropyridin-3-yl | cyclopropyl | O | H | H | chloro | H | H | H | H | H | rel-(1R,2R) | 2.10^{[c]}; |
| | | | | | | | | | | | | | 2.14^{[b]} |
| 275 | 2-chlorophenyl | cyclopropyl | O | H | H | chloro | H | H | H | H | H | rel-(1R,2R) | 2.71^{[c]}; |
| | | | | | | | | | | | | | 2.77^{[b]} |
| 276 | 2-methylphenyl | cyclopropyl | O | H | H | chloro | H | H | H | H | H | rel-(1R,2R) | 2.70^{[c]}; |
| | | | | | | | | | | | | | 2.76^{[b]} |
| 277 | 1,3-dimethyl-1H-pyrazol-4-yl | cyclopentyl | O | H | H | Cl | H | H | H | H | H | | 2.31^{[b]} |
| 278 | 2-chlorophenyl | cyclopentyl | O | H | H | Cl | H | H | H | H | H | | 3.45^{[b]} |
| 279 | 2-(trifluoromethyl)phenyl | cyclopentyl | O | H | H | Cl | H | H | H | H | H | | 3.64^{[b]} |
| 280 | 2-iodophenyl | cyclopentyl | O | H | H | Cl | H | H | H | H | H | | 3.64^{[b]} |
| 281 | 2-chloropyridin-3-yl | cyclopentyl | O | H | H | Cl | H | H | H | H | H | | 2.81^{[b]} |
| 282 | 2,6-difluorophenyl | cyclopentyl | O | H | H | Cl | H | H | H | H | H | | 3.28^{[b]} |
| 283 | 2-(trifluoromethyl)pyridin-3-yl | cyclopentyl | O | H | H | Cl | H | H | H | H | H | | 2.83^{[b]} |
| 284 | 2,4-dimethyl-1,3-thiazol-5-yl | cyclopentyl | O | H | H | Cl | H | H | H | H | H | | 3.11^{[b]} |
| 285 | 2-iodophenyl | cyclopentyl | O | H | H | H | H | trifluoromethyl | H | H | H | | 3.81^{[b]} |
| 286 | 2-(trifluoromethyl)phenyl | cyclopentyl | O | H | H | H | H | trifluoromethyl | H | H | H | | 3.79^{[b]} |
| 287 | 3-(difluoromethyl)-1-methyl-1H-pyrazol-4-yl | cyclopentyl | O | H | H | H | H | trifluoromethyl | H | H | H | | 3.06^{[b]} |
| 288 | 3-(difluoromethyl)-1-methyl-1H-pyrazol-4-yl | cyclohexyl | O | H | H | H | H | trifluoromethyl | H | H | H | rel-(1R,2S) | 3.56^{[b]} |
| 289 | 2-(trifluoromethyl)pyridin-3-yl | cyclopentyl | O | trifluoromethyl | H | H | H | H | H | H | H | rel-(1R,2S) | 3.27^{[b]} |
| 290 | 2-(trifluoromethyl)pyridin-3-yl | cyclopentyl | O | trifluoromethyl | H | H | H | H | H | H | H | rel-(1R,2R) | 3.29^{[b]} |
| 291 | 2,4-dimethyl-1,3-thiazol-5-yl | cyclopentyl | O | H | H | H | H | Cl | H | H | H | | 2.73^{[b]} |
| 292 | 1,3-dimethyl-1H-pyrazol-4-yl | cyclopentyl | O | H | H | H | H | Cl | H | H | H | | 2.24^{[b]} |
| 293 | 2-chloropyridin-3-yl | cyclopentyl | O | H | H | H | H | Cl | H | H | H | | 2.64^{[b]} |
| 294 | 2-chlorophenyl | cyclopentyl | O | H | H | H | H | Cl | H | H | H | | 3.42^{[b]} |
| 295 | 2-iodophenyl | cyclopentyl | O | H | H | H | H | Cl | H | H | H | | 3.51^{[b]} |
| 296 | 2-(trifluoromethyl)phenyl | cyclopentyl | O | H | H | H | H | Cl | H | H | H | | 3.55^{[b]} |
| 297 | 2-(trifluoromethyl)pyridin-3-yl | cyclopentyl | O | H | H | H | H | Cl | H | H | H | | 3.05^{[b]} |
| 298 | pyridin-3-yl | cyclopentyl | O | H | H | trifluoromethyl | H | H | H | H | H | rel-(1R,2R) | 2.46^{[c]}; |
| | | | | | | | | | | | | | 2.46^{[b]} |
| 299 | pyridin-3-ylcarbonyl | cyclopentyl | O | H | H | trifluoromethyl | H | H | H | H | methyl | | 2.81^{[c]}; |
| | | | | | | | | | | | | | 2.67^{[b]} |
| 300 | 2-chlorophenyl | cyclopentyl | O | H | H | H | H | trifluoromethyl | H | H | H | | 3.56^{[b]} |
| 301 | 2-chloropyridin-3-yl | cyclopentyl | O | H | H | H | H | trifluoromethyl | H | H | H | | 2.90^{[b]} |
| 302 | 2-(trifluoromethyl)phenyl | cyclopentyl | O | H | H | trifluoromethyl | H | H | H | H | H | rel-(1R,2R) | 3.79^{[c]}; |
| | | | | | | | | | | | | | 3.83^{[b]} |
| 303 | 2-bromophenyl | cyclopentyl | O | H | H | trifluoromethyl | H | H | H | H | H | rel-(1R,2R) | 3.68^{[c]}; |
| | | | | | | | | | | | | | 3.73^{[b]} |
| 304 | 2,6-difluorophenyl | cyclopentyl | O | H | H | trifluoromethyl | H | H | H | H | H | rel-(1R,2R) | 3.47^{[c]}; |
| | | | | | | | | | | | | | 3.51^{[b]} |
| 305 | 2-chlorophenyl | cyclopentyl | O | H | H | trifluoromethyl | H | H | H | H | H | | 3.63^{[c]}; |
| | | | | | | | | | | | | | 3.68^{[b]} |
| 306 | 2-nitrophenyl | cyclopentyl | O | H | H | trifluoromethyl | H | H | H | H | H | rel-(1R,2R) | 3.27^{[c]}; |
| | | | | | | | | | | | | | 3.31^{[b]} |
| 307 | 2-(trifluoromethyl)phenyl | cyclopentyl | O | H | H | trifluoromethyl | H | H | H | H | methyl | | 4.40^{[c]}; |
| | | | | | | | | | | | | | 4.46^{[b]} |
| 308 | 2,4-dimethyl-1,3-thiazol-5-yl | cyclopentyl | O | H | H | H | H | trifluoromethyl | H | H | H | | 2.97^{[b]} |
| 309 | 2-(trifluoromethyl)pyridin-3-yl | cyclopentyl | O | trifluoromethyl | H | H | H | H | H | H | H | | 3.22^{[b]} |
| 310 | 1,3-dimethyl-1H-pyrazol-4-yl | cyclopentyl | O | H | H | H | H | trifluoromethyl | H | H | H | | 2.47^{[b]} |
| 311 | 2-chlorophenyl | cyclopentyl | O | F | H | H | H | F | H | H | H | | 3.24^{[b]} |
| 312 | 2,4-dimethyl-1,3-thiazol-5-yl | cyclopentyl | O | F | H | H | H | F | H | H | H | | 2.65^{[b]} |
| 313 | 2-(trifluoromethyl)pyridin-3-yl | cyclopentyl | O | F | H | H | H | F | H | H | H | | 2.87^{[b]} |
| 314 | 2,6-difluorophenyl | cyclopentyl | O | F | H | H | H | F | H | H | H | | 3.05^{[b]} |
| 315 | 2-chloropyridin-3-yl | cyclopentyl | O | H | H | trifluoromethyl | H | H | H | H | H | rel-(1R,2R) | 2.98^{[c]}; |
| | | | | | | | | | | | | | 3.04^{[b]} |
| 316 | 2-chlorophenyl | cyclopentyl | O | H | H | H | H | F | H | H | H | rel-(1R,2R) | 3.08^{[c]}; |
| | | | | | | | | | | | | | 3.12^{[b]} |
| 317 | 2-chlorophenyl | cyclopentyl | O | H | H | isopropoxy | H | H | H | H | H | rel-(1R,2R) | 3.73^{[c]}; |
| | | | | | | | | | | | | | 3.74^{[b]} |
| 318 | 2-nitrophenyl | cyclopentyl | O | H | H | isopropoxy | H | H | H | H | H | rel-(1R,2R) | 3.25^{[c]}; |
| | | | | | | | | | | | | | 3.30^{[b]} |
| 319 | 2-bromophenyl | cyclopentyl | O | H | H | H | H | F | H | H | H | rel-(1R,2R) | 3.14^{[c]}; |
| | | | | | | | | | | | | | 3.18^{[b]} |
| 320 | 2,6-difluorophenyl | cyclopentyl | O | H | H | H | H | trifluoromethyl | H | H | H | | 3.38^{[b]} |
| 321 | 2-chloropyridin-3-yl | cyclopentyl | O | F | H | H | H | F | H | H | H | | 2.51^{[b]} |
| 322 | 2-iodophenyl | cyclopentyl | O | F | H | H | H | F | H | H | H | | 3.40^{[b]} |
| 323 | 2-(trifluoromethyl)phenyl | cyclopentyl | O | F | H | H | H | F | H | H | H | | 3.43^{[b]} |
| 324 | 1,3-dimethyl-1H-pyrazol-4-yl | cyclopentyl | O | F | H | H | H | F | H | H | H | | 2.13^{[b]} |
| 325 | 2-(trifluoromethyl)phenyl | cyclopentyl | O | H | H | isopropoxy | H | H | H | H | H | rel-(1R,2R) | 3.84^{[c]}; |
| | | | | | | | | | | | | | 3.90^{[b]} |
| 326 | 2-chloropyridin-3-yl | cyclopentyl | O | H | H | isopropoxy | H | H | H | H | H | rel-(1R,2R) | 2.99^{[c]}; |
| | | | | | | | | | | | | | 3.06^{[b]} |
| 327 | 2-bromophenyl | cyclopentyl | O | H | H | isopropoxy | H | H | H | H | H | rel-(1R,2R) | 3.74^{[c]}; |
| | | | | | | | | | | | | | 3.81^{[b]} |
| 328 | 2,6-difluorophenyl | cyclopentyl | O | H | H | H | H | F | H | H | H | rel-(1R,2R) | 2.90^{[c]}; |
| | | | | | | | | | | | | | 2.95^{[b]} |
| 329 | 2,6-difluorophenyl | cyclopentyl | O | H | H | isopropoxy | H | H | H | H | H | rel-(1R,2R) | 3.49^{[c]}; |
| | | | | | | | | | | | | | 3.55^{[b]} |
| 330 | 3-(trifluoromethyl)pyrazin-2-yl | cyclopentyl | O | H | H | trifluoromethyl | H | H | H | H | H | rel-(1R,2R) | 3.45^{[c]}; |
| | | | | | | | | | | | | | 3.55^{[b]} |
| 331 | 3-(trifluoromethyl)pyridin-2-yl | cyclopentyl | O | H | H | trifluoromethyl | H | H | H | H | H | rel-(1R,2R) | 3.28^{[c]}; |
| | | | | | | | | | | | | | 3.36^{[b]} |
| 332 | 2-iodophenyl | cyclopentyl | O | H | H | H | H | F | H | H | H | rel-(1R,2R) | 3.28^{[c]}; |
| | | | | | | | | | | | | | 3.30^{[b]} |
| 333 | 2-iodophenyl | cyclopentyl | O | H | H | isopropoxy | H | H | H | H | H | rel-(1R,2R) | 3.89^{[c]}; |
| | | | | | | | | | | | | | 3.92^{[b]} |
| 334 | pyridin-2-yl | cyclopentyl | O | H | H | isopropoxy | H | H | H | H | H | rel-(1R,2R) | 2.46^{[c]}; |
| | | | | | | | | | | | | | 2.41^{[b]} |
| 335 | 3-chloropyrazin-2-yl | cyclopentyl | O | H | H | isopropoxy | H | H | H | H | H | rel-(1R,2R) | 3.21^{[c]}; |
| | | | | | | | | | | | | | 3.23^{[b]} |
| 336 | 3-chloropyrazin-2-yl | cyclopentyl | O | H | H | H | H | F | H | H | H | rel-(1R,2R) | 2.55^{[c]}; |
| | | | | | | | | | | | | | 2.61^{[b]} |
| 337 | 5-iodo-2-thienyl | cyclopentyl | O | H | H | H | H | f | H | H | H | rel-(1R,2R) | 3.58^{[c]}; |
| | | | | | | | | | | | | | 3.70^{[b]} |
| 338 | 5-iodo-2-thienyl | cyclopentyl | O | H | H | isopropoxy | H | H | H | H | H | rel-(1R,2R) | 4.27^{[c]}; |
| | | | | | | | | | | | | | 4.43^{[b]} |
| 339 | pyridin-2-yl | cyclopentyl | O | F | H | H | H | H | H | H | H | rel-(1R,2R) | 1.95^{[c]}; |
| | | | | | | | | | | | | | 1.86^{[b]} |
| 340 | 3-(trifluoromethyl)pyrazin-2-yl | cyclopentyl | O | H | H | isopropoxy | H | H | H | H | H | rel-(1R,2R) | 3.55^{[c]}; |
| | | | | | | | | | | | | | 3.62^{[b]} |
| 341 | 3-(trifluoromethyl)pyrazin-2-yl | cyclopentyl | O | H | H | H | H | F | H | H | H | rel-(1R,2R) | 2.94^{[c]}; |
| | | | | | | | | | | | | | 3.00^{[b]} |
| 342 | 2-methyl-4-(trifluoromethyl)-1,3-thiazol-5-yl | cyclopentyl | O | H | H | isopropoxy | H | H | H | H | H | rel-(1R,2R) | 3.71^{[c]}; |
| | | | | | | | | | | | | | 3.80^{[b]} |
| 343 | 2-methyl-4-(trifluoromethyl)-1,3-thiazol-5-yl | cyclopentyl | O | F | H | H | H | H | H | H | H | rel-(1R,2R) | 3.07^{[c]}; |
| | | | | | | | | | | | | | 3.18^{[b]} |
| 344 | 2,4-dimethyl-1,3-thiazol-5-yl | cyclopentyl | O | H | H | H | H | Br | H | H | H | | 2.86^{[b]} |
| 345 | 1,3-dimethyl-1H-pyrazol-4-yl | cyclopentyl | O | H | H | H | H | Br | H | H | H | | 3.69^{[b]} |
| 346 | 2-(trifluoromethyl)pyridin-3-yl | cyclopentyl | O | H | H | H | H | Br | H | H | H | | 3.13^{[b]} |
| 347 | 2,6-difluorophenyl | cyclopentyl | O | H | H | H | H | Br | H | H | H | | 3.35^{[b]} |
| 348 | 2-chloropyridin-3-yl | cyclopentyl | O | H | H | H | H | Br | H | H | H | | 2.80^{[b]} |
| 349 | 2-chlorophenyl | cyclopentyl | O | H | H | H | H | Br | H | H | H | | 3.52^{[b]} |
| 350 | 2,6-difluorophenyl | cyclopentyl | O | H | H | Cl | H | Cl | H | H | H | | 3.73^{[b]} |
| 351 | 2-chlorophenyl | cyclopentyl | O | Cl | H | Cl | H | H | H | H | H | | 3.87^{[b]} |
| 352 | 2-iodophenyl | cyclopentyl | O | Cl | H | Cl | H | H | H | H | H | | 4.04^{[b]} |
| 353 | 2-(trifluoromethyl)phenyl | cyclopentyl | O | Cl | H | Cl | H | H | H | H | H | | 4.04^{[b]} |
| 354 | 2,4-dimethyl-1,3-thiazol-5-yl | cyclopentyl | O | H | H | Cl | H | Cl | H | H | H | | 3.24^{[b]} |
| 355 | 2-(trifluoromethyl)phenyl | cyclopentyl | O | H | H | H | H | Br | H | H | H | | 3.71^{[b]} |
| 356 | 2-chloropyridin-3-yl | cyclopentyl | O | Cl | H | Cl | H | H | H | H | H | | 3.20^{[b]} |
| 357 | 2-iodophenyl | cyclopentyl | O | H | H | H | H | Br | H | H | H | | 3.67^{[b]} |
| 358 | 2-(trifluoromethyl)pyridin-3-yl | cyclopentyl | O | H | H | Cl | H | Cl | H | H | H | | 3.54^{[b]} |
| 359 | 1,3-dimethyl-1H-pyrazol-4-yl | cyclopentyl | O | H | H | Cl | H | Cl | H | H | H | | 2.67^{[b]} |
| 360 | 3-iodo-2-thienyl | cyclopentyl | O | H | H | trifluoromethyl | H | H | H | H | H | rel-(1R,2R) | 4.16^{[c]}; |
| | | | | | | | | | | | | | 4.21^{[b]} |
| 361 | 2-(trifluoromethyl)pyridin-3-yl | cyclopentyl | O | H | H | H | H | Cl | H | H | H | rel-(1R,2R) | 3.01^{[b]} |
| 362 | 2-(trifluoromethyl)pyridin-3-yl | cyclopentyl | O | F | H | H | H | F | H | H | H | rel-(1R,2R) | 2.86^{[b]} |
| 363 | 2-iodophenyl | cyclopentyl | O | H | H | trifluoromethyl | H | H | H | H | H | rel-(1R,2R) | 3.79^{[c]}; |
| | | | | | | | | | | | | | 3.86^{[b]} |
| 364 | 2-(trifluoromethyl)phenyl | cyclohexyl | O | H | H | Cl | H | H | H | H | H | | 3.84^{[b]} |
| 365 | 2-(trifluoromethyl)phenyl | cyclohexyl | O | H | H | trifluoromethyl | H | H | H | H | H | | 4.05^{[c]}; |
| | | | | | | | | | | | | | 4.12^{[b]} |
| 366 | 2,4-dimethyl-1,3-thiazol-5-yl | cyclohexyl | O | H | H | Cl | H | H | H | H | H | | 3.03^{[b]} |
| 367 | 1,3-dimethyl-1H-pyrazol-4-yl | cyclohexyl | O | H | H | Cl | H | H | H | H | H | | 2.49^{[b]} |
| 368 | 2-(trifluoromethyl)pyridin-3-yl | cyclohexyl | O | H | H | Cl | H | H | H | H | H | | 3.31^{[b]} |
| 369 | 2,6-difluorophenyl | cyclohexyl | O | H | H | Cl | H | H | H | H | H | | 3.50^{[b]} |
| 370 | 2-chloropyridin-3-yl | cyclohexyl | O | H | H | Cl | H | H | H | H | H | | 2.99^{[b]} |
| 371 | 2-chlorophenyl | cyclohexyl | O | H | H | Cl | H | H | H | H | H | | 3.65^{[b]} |
| 372 | 2-iodophenyl | cyclohexyl | O | H | H | Cl | H | H | H | H | H | | 3.84^{[b]} |
| 373 | 2-iodophenyl | cyclohexyl | O | H | H | H | H | trifluoromethyl | H | H | H | rel-(1R,2S) | 4.22^{[b]} |
| 374 | 2-(trifluoromethyl)phenyl | cyclohexyl | O | H | H | H | H | trifluoromethyl | H | H | H | rel-(1R,2S) | 4.21^{[b]} |
| 375 | 2-(trifluoromethyl)pyridin-3-yl | cyclohexyl | O | H | H | H | H | trifluoromethyl | H | H | H | rel-(1R,2S) | 3.68^{[b]} |
| 376 | 2,4-dimethyl-1,3-thiazol-5-yl | cyclohexyl | O | H | H | H | H | Cl | H | H | H | | 3.24^{[b]} |
| 377 | 1,3-dimethyl-1H-pyrazol-4-yl | cyclohexyl | O | H | H | H | H | Cl | H | H | H | | 2.64^{[b]} |
| 378 | 2-(trifluoromethyl)pyridin-3-yl | cyclohexyl | O | H | H | H | H | Cl | H | H | H | | 3.45^{[b]} |
| 379 | 2,6-difluorophenyl | cyclohexyl | O | H | H | H | H | Cl | H | H | H | | 3.67^{[b]} |
| 380 | 2-chloropyridin-3-yl | cyclohexyl | O | H | H | H | H | Cl | H | H | H | | 3.16^{[b]} |
| 381 | 2-chlorophenyl | cyclohexyl | O | H | H | H | H | Cl | H | H | H | | 3.88^{[b]} |
| 382 | 2-iodophenyl | cyclohexyl | O | H | H | H | H | Cl | H | H | H | | 4.02^{[b]} |
| 383 | 2-(trifluoromethyl)phenyl | cyclohexyl | O | H | H | H | H | Cl | H | H | H | | 4.01^{[b]} |
| 384 | 1,3-dimethyl-1H-pyrazol-4-yl | cyclohexyl | O | H | H | H | H | trifluoromethyl | H | H | H | | 2.87^{[b]} |
| 385 | 2-(trifluoromethyl)pyridin-3-yl | cyclohexyl | O | H | H | H | H | trifluoromethyl | H | H | H | | 3.61^{[b]} |
| 386 | 2,6-difluorophenyl | cyclohexyl | O | H | H | H | H | trifluoromethyl | H | H | H | | 3.80^{[b]} |
| 387 | 2-chloropyridin-3-yl | cyclohexyl | O | H | H | H | H | trifluoromethyl | H | H | H | | 3.33^{[b]} |
| 388 | 2-chlorophenyl | cyclohexyl | O | H | H | H | H | trifluoromethyl | H | H | H | | 4.02^{[b]} |
| 389 | 2-iodophenyl | cyclohexyl | O | H | H | H | H | trifluoromethyl | H | H | H | | 4.13^{[b]} |
| 390 | 2-(trifluoromethyl)phenyl | cyclohexyl | O | H | H | H | H | trifluoromethyl | H | H | H | | 4.11^{[b]} |
| 391 | 2,6-difluorophenyl | cyclopentyl | O | H | H | H | H | Cl | H | H | H | | 3.18^{[b]} |
| 392 | 2,4-dimethyl-1,3-thiazol-5-yl | cyclohexyl | O | H | H | H | H | trifluoromethyl | H | H | H | | 3.49^{[b]} |
| 393 | 1,3-dimethyl-1H-pyrazol-4-yl | cyclohexyl | O | F | H | H | H | F | H | H | H | | 2.51^{[b]} |
| 394 | 2,4-dimethyl-1,3-thiazol-5-yl | cyclohexyl | O | F | H | H | H | F | H | H | H | | 3.16^{[b]} |
| 395 | 2,6-difluorophenyl | cyclohexyl | O | F | H | H | H | F | H | H | H | | 3.44^{[b]} |
| 396 | 2-chlorophenyl | cyclohexyl | O | F | H | H | H | F | H | H | H | | 3.68^{[b]} |
| 397 | 2-chloropyridin-3-yl | cyclohexyl | O | F | H | H | H | F | H | H | H | | 2.99^{[b]} |
| 398 | 2-(trifluoromethyl)pyridin-3-yl | cyclohexyl | O | F | H | H | H | F | H | H | H | | 3.26^{[b]} |
| 399 | 2-(trifluoromethyl)phenyl | cyclohexyl | O | F | H | H | H | F | H | H | H | | 3.87^{[b]} |
| 400 | 2-iodophenyl | cyclohexyl | O | F | H | H | H | F | H | H | H | | 3.84^{[b]} |
| 401 | 2,4-dimethyl-1,3-thiazol-5-yl | cyclohexyl | O | H | H | H | H | Br | H | H | H | | 3.35^{[b]} |
| 402 | 1,3-dimethyl-1H-pyrazol-4-yl | cyclohexyl | O | H | H | H | H | Br | H | H | H | | 2.74^{[b]} |
| 403 | 2-(trifluoromethyl)pyridin-3-yl | cyclohexyl | O | H | H | H | H | Br | H | H | H | | 3.53^{[b]} |
| 404 | 2,6-difluorophenyl | cyclohexyl | O | H | H | H | H | Br | H | H | H | | 3.78^{[b]} |
| 405 | 2-chlorophenyl | cyclohexyl | O | H | H | H | H | Br | H | H | H | | 3.97^{[b]} |
| 406 | 2-iodophenyl | cyclohexyl | O | H | H | H | H | Br | H | H | H | | 4.12^{[b]} |
| 407 | 2-(trifluoromethyl)phenyl | cyclohexyl | O | H | H | H | H | Br | H | H | H | | 4.10^{[b]} |
| 408 | 2-chloropyridin-3-yl | cyclohexyl | O | Cl | H | Cl | H | H | H | H | H | | 3.76^{[b]} |
| 409 | 2-chlorophenyl | cyclohexyl | O | Cl | H | Cl | H | H | H | H | H | | 4.47^{[b]} |
| 410 | 2,4-dimethyl-1,3-thiazol-5-yl | cyclohexyl | O | H | H | Cl | H | Cl | H | H | H | | 3.88^{[b]} |
| 411 | 2,6-difluorophenyl | cyclohexyl | O | H | H | Cl | H | Cl | H | H | H | | 4.26^{[b]} |
| 412 | 2-(trifluoromethyl)phenyl | cyclohexyl | O | Cl | H | Cl | H | H | H | H | H | | 4.49^{[b]} |
| 413 | 2-chloropyridin-3-yl | cyclohexyl | O | H | H | H | H | Br | H | H | H | | 3.25^{[b]} |
| 414 | 2-(trifluoromethyl)pyridin-3-yl | cyclohexyl | O | H | H | Cl | H | Cl | H | H | H | | 4.03^{[b]} |
| 415 | 2-iodophenyl | cyclohexyl | O | Cl | H | Cl | H | H | H | H | H | | 4.57^{[b]} |
| 416 | 1,3-dimethyl-1H-pyrazol-4-yl | cyclohexyl | O | H | H | Cl | H | Cl | H | H | H | | 3.19^{[b]} |
| 418 | 2-(trifluoromethyl)pyridin-3-yl | cycloheptyl | O | H | H | H | H | trifluoromethyl | H | H | H | rel-(1R,2S) | 3.94^{[b]} |
| 419 | 2-iodophenyl | cycloheptyl | O | H | H | H | H | trifluoromethyl | H | H | H | rel-(1R,2S) | 4.59^{[b]} |
| 420 | 2-(trifluoromethyl)phenyl | cycloheptyl | O | H | H | H | H | trifluoromethyl | H | H | H | rel-(1R,2S) | 4.55^{[b]} |
| 421 | 3-(difluoromethyl)-1-methyl-1H-pyrazol-4-yl | cycloheptyl | O | H | H | H | H | trifluoromethyl | H | H | H | rel-(1R,2S) | 3.69^{[b]} |
| 422 | 2-fluorophenyl | cyclopropyl | O | H | H | Cl | H | H | H | H | H | | 3.07^{[c]}; |
| | | | | | | | | | | | | | 3.12^{[b]} |
| 423 | 2-bromophenyl | cyclopropyl | O | H | H | Cl | H | H | H | H | H | | 3.07^{[c]}; |
| | | | | | | | | | | | | | 3.13^{[b]} |
| 424 | 2-(trifluoromethyl)phenyl | cyclopropyl | O | H | H | Cl | H | Cl | H | H | H | rel-(1R,2R) | 3.46^{[b]} |
| 425 | 2-chloropyridin-3-yl | cyclopropyl | O | H | H | Cl | H | Cl | H | H | H | rel-(1R,2R) | 2.62^{[b]} |
| 426 | phenyl | cyclobutyl | O | H | H | H | H | trifluoromethyl | H | H | H | rel-(1R,2R) | 3.08^{[b]} |
| 427 | 2-iodophenyl | cyclobutyl | O | H | H | H | H | trifluoromethyl | H | H | H | rel-(1R,2R) | 3.44^{[b]} |
| 428 | 2-iodophenyl | cyclobutyl | O | H | H | H | H | trifluoromethyl | H | H | H | rel-(1R,2R) | 3.42^{[b]} |
| 429 | 4-chlorophenyl | cyclobutyl | O | H | H | H | H | trifluoromethyl | H | H | H | rel-(1R,2R) | 3.56^{[b]} |
| 430 | 2,6-difluorophenyl | cyclobutyl | O | H | H | H | H | trifluoromethyl | H | H | H | rel-(1R,2R) | 3.09^{[b]} |
| 432 | 2-chlorophenyl | cyclobutyl | O | H | H | H | H | trifluoromethyl | H | H | H | rel-(1R,2R) | 3.30^{[b]} |
| 433 | 2-chlorophenyl | cyclobutyl | O | H | H | H | H | trifluoromethyl | H | H | H | rel-(1R,2R) | 3.01^{[b]} |
| 434 | 2-(trifluoromethyl)phenyl | cyclobutyl | O | H | H | H | H | trifluoromethyl | H | H | H | rel-(1R,2R) | 3.44^{[b]} |
| 436 | 3-chlorophenyl | cyclobutyl | O | H | H | H | H | trifluoromethyl | H | H | H | rel-(1R,2R) | 3.59^{[b]} |
| 437 | 3-chloro-5-(trifluoromethyl)pyridin-2-yl | cyclobutyl | O | H | H | H | H | trifluoromethyl | H | H | H | rel-(1R,2R) | 3.88^{[b]} |
| 438 | 3-chloropyridin-2-yl | cyclobutyl | O | F | H | H | H | F | H | H | H | rel-(1R,2R) | 2.73^{[b]} |
| 439 | 2,6-difluorophenyl | cyclobutyl | O | F | H | H | H | F | H | H | H | rel-(1R,2R) | 2.83^{[b]} |
| 440 | 2-iodophenyl | cyclobutyl | O | F | H | H | H | F | H | H | H | rel-(1R,2R) | 3.13^{[b]} |
| 441 | 2,4-dichlorophenyl | cyclobutyl | O | F | H | H | H | F | H | H | H | rel-(1R,2R) | 3.49^{[b]} |
| 442 | 3-chlorophenyl | cyclobutyl | O | F | H | H | H | F | H | H | H | rel-(1R,2R) | 3.26^{[b]} |
| 443 | 2-chlorophenyl | cyclobutyl | O | F | H | H | H | F | H | H | H | rel-(1R,2R) | 2.97^{[b]} |
| 444 | 4-chlorophenyl | cyclobutyl | O | F | H | H | H | F | H | H | H | rel-(1R,2R) | 3.20^{[b]} |
| 445 | 3-chlorophenyl | cyclobutyl | O | H | H | H | H | Cl | H | H | H | rel-(1R,2S) | 3.75^{[b]} |
| 446 | 4-chlorophenyl | cyclobutyl | O | H | H | H | H | Cl | H | H | H | rel-(1R,2S) | 3.70^{[b]} |
| 447 | phenyl | cyclobutyl | O | H | H | H | H | Cl | H | H | H | rel-(1R,2S) | 3.14^{[b]} |
| 448 | 3-chlorophenyl | cyclobutyl | O | H | H | H | H | Cl | H | H | H | rel-(1R,2R) | 3.36^{[b]} |
| 449 | phenyl | cyclobutyl | O | H | H | H | H | Cl | H | H | H | rel-(1R,2R) | 2.81^{[b]} |
| 450 | phenyl | cyclobutyl | O | Cl | H | Cl | H | H | H | H | H | rel-(1R,2S) | 3.75^{[b]} |
| 451 | phenyl | cyclobutyl | O | Cl | H | Cl | H | H | H | H | H | rel-(1R,2R) | 3.30^{[b]} |
| 452 | 4-chlorophenyl | cyclobutyl | O | Cl | H | Cl | H | H | H | H | H | rel-(1R,2S) | 4.33^{[b]} |
| 453 | 4-chlorophenyl | cyclobutyl | O | Cl | H | Cl | H | H | H | H | H | rel-(1R,2R) | 3.85^{[b]} |
| 454 | 3-chlorophenyl | cyclobutyl | O | Cl | H | Cl | H | H | H | H | H | rel-(1R,2S) | 4.40^{[b]} |
| 455 | 4-chlorophenyl | cyclobutyl | O | H | H | H | H | Cl | H | H | H | rel-(1R,2R) | 3.35^{[b]} |
| 456 | 2-iodophenyl | cyclobutyl | O | Cl | H | Cl | H | H | H | H | H | rel-(1R,2R) | 3.75^{[b]} |
| 457 | 3-chlorophenyl | cyclobutyl | O | Cl | H | Cl | H | H | H | H | H | rel-(1R,2R) | 3.87^{[b]} |
| 458 | 3-chloropyridin-2-yl | cyclobutyl | O | H | H | H | H | trifluoromethyl | H | H | H | rel-(1R,2S) | 3.22^{[b]} |
| 459 | 2-(difluoromethyl)phenyl | cyclopropyl | O | H | H | Cl | H | H | H | H | H | rel-(1R,2R) | 2.88^{[c]}; |
| | | | | | | | | | | | | | 2.90^{[b]} |
| 460 | 2-iodo-3-thienyl | cyclopropyl | O | H | H | Cl | H | H | H | H | H | rel-(1R,2R) | 2.93^{[c]}; |
| | | | | | | | | | | | | | 2.94^{[b]} |
| 461 | 3-chloropyrazin-2-yl | cyclopropyl | O | H | H | Cl | H | H | H | H | H | rel-(1R,2R) | 2.27^{[c]}; |
| | | | | | | | | | | | | | 2.29^{[b]} |
| 462 | 3-(trifluoromethyl)pyridin-2-yl | cyclopropyl | O | H | H | Cl | H | H | H | H | H | rel-(1R,2R) | 2.81^{[c]}; |
| | | | | | | | | | | | | | 2.85^{[b]} |
| 463 | 2-chloro-6-fluorophenyl | cyclopropyl | O | H | H | Cl | H | H | H | H | H | rel-(1R,2R) | 2.75^{[c]}; |
| | | | | | | | | | | | | | 2.83^{[b]} |
| 464 | 2-fluoro-6-iodophenyl | cyclopropyl | O | H | H | Cl | H | H | H | H | H | rel-(1R,2R) | 2.99^{[c]}; |
| | | | | | | | | | | | | | 3.02^{[b]} |
| 465 | 2-(difluoromethyl)pyridin-3-yl | cyclopropyl | O | H | H | Cl | H | H | H | H | H | rel-(1R,2R) | 2.14^{[c]}; |
| | | | | | | | | | | | | | 2.15^{[b]} |
| 466 | 2,4,6-trifluorophenyl | cyclopropyl | O | H | H | Cl | H | H | H | H | H | rel-(1R,2R) | 2.78^{[c]}; |
| | | | | | | | | | | | | | 2.80^{[b]} |
| 467 | 2-nitrophenyl | cyclopropyl | O | H | H | Cl | H | H | H | H | H | rel-(1R,2R) | 2.45^{[c]}; |
| | | | | | | | | | | | | | 2.59^{[b]} |
| 468 | 3-(difluoromethyl)-1-methyl-1H-pyrazol-4-yl | cyclobutyl | O | H | H | Cl | H | Cl | H | H | H | rel-(1R,2S) | 3.26^{[b]} |
| 469 | 3-(difluoromethyl)-1-methyl-1H-pyrazol-4-yl | cyclobutyl | O | H | H | Cl | H | Cl | H | H | H | rel-(1R,2R) | 2.95^{[b]} |
| 470 | 2-(trifluoromethyl)pyridin-3-yl | cyclobutyl | O | Cl | H | Cl | H | H | H | H | H | rel-(1R,2S) | 3.45^{[b]} |
| 471 | 2-bromophenyl | cyclobutyl | O | H | H | Cl | H | Cl | H | H | H | rel-(1R,2S) | 3.92^{[b]} |
| 472 | 2-bromophenyl | cyclobutyl | O | H | H | Cl | H | Cl | H | H | H | rel-(1R,2R) | 3.60^{[b]} |
| 473 | 2,6-difluorophenyl | cyclobutyl | O | Cl | H | Cl | H | H | H | H | H | rel-(1R,2R) | 3.36^{[b]} |
| 474 | 2-(trifluoromethyl)phenyl | cyclobutyl | O | Cl | H | Cl | H | H | H | H | H | rel-(1R,2S) | 4.02^{[b]} |
| 476 | 2-(trifluoromethyl)phenyl | cyclobutyl | O | Cl | H | Cl | H | H | H | H | H | rel-(1R,2R) | 3.72^{[b]} |
| 477 | 3-chlorophenyl | cyclobutyl | O | H | H | F | H | trifluoromethyl | H | H | H | rel-(1R,2S) | 4.02^{[b]} |
| 478 | 3-chloropyridin-2-yl | cyclobutyl | O | H | H | F | H | trifluoromethyl | H | H | H | rel-(1R,2S) | 3.39^{[b]} |
| 479 | 3-chloropyridin-2-yl | cyclobutyl | O | H | H | F | H | trifluoromethyl | H | H | H | rel-(1R,2R) | 3.11^{[b]} |
| 480 | 3-chloro-5-(trifluoromethyl)pyridin-2-yl | cyclobutyl | O | H | H | F | H | trifluoromethyl | H | H | H | rel-(1R,2S) | 4.23^{[b]} |
| 481 | 2-(trifluoromethyl)pyridin-3-yl | cyclobutyl | O | Cl | H | Cl | H | H | H | H | H | rel-(1R,2R) | 3.20^{[b]} |
| 482 | 2,6-difluorophenyl | cyclobutyl | O | Cl | H | Cl | H | H | H | H | H | rel-(1R,2S) | 3.70^{[b]} |
| 483 | 3-chloro-5-(trifluoromethyl)pyridin-2-yl | cyclobutyl | O | H | H | F | H | trifluoromethyl | H | H | H | rel-(1R,2R) | 3.97^{[b]} |
| 484 | 3-(difluoromethyl)-1-methyl-1H-pyrazol-4-yl | cyclobutyl | O | trifluoromethyl | H | F | H | H | H | H | H | rel-(1R,2S) | 3.02^{[b]} |
| 485 | 3-(difluoromethyl)-1-methyl-1H-pyrazol-4-yl | cyclobutyl | O | trifluoromethyl | H | F | H | H | H | H | H | rel-(1R,2R) | 2.84^{[b]} |
| 486 | 3-(difluoromethyl)-1-methyl-1H-pyrazol-4-yl | cyclobutyl | O | F | H | H | H | F | H | H | H | rel-(1R,2R) | 2.42^{[b]} |
| 487 | 2-(trifluoromethyl)pyridin-3-yl | cyclobutyl | O | H | H | F | H | trifluoromethyl | H | H | H | rel-(1R,2S) | 3.20^{[b]} |
| 489 | 2-(trifluoromethyl)pyridin-3-yl | cyclobutyl | O | H | H | F | H | trifluoromethyl | H | H | H | rel-(1R,2R) | 3.02^{[b]} |
| 491 | 2-bromophenyl | cyclobutyl | O | H | H | F | H | trifluoromethyl | H | H | H | rel-(1R,2R) | 3.39^{[b]} |
| 492 | 2,6-difluorophenyl | cyclobutyl | O | H | H | F | H | trifluoromethyl | H | H | H | rel-(1R,2S) | 3.38^{[b]} |
| 493 | 2,6-difluorophenyl | cyclobutyl | O | H | H | F | H | trifluoromethyl | H | H | H | rel-(1R,2R) | 3.18^{[b]} |
| 494 | 2-(trifluoromethyl)phenyl | cyclobutyl | O | H | H | F | H | trifluoromethyl | H | H | H | rel-(1R,2S) | 3.72^{[b]} |
| 495 | 2-(trifluoromethyl)phenyl | cyclobutyl | O | H | H | F | H | trifluoromethyl | H | H | H | rel-(1R,2R) | 3.51^{[b]} |
| 496 | 2-(trifluoromethyl)phenyl | cyclobutyl | O | H | H | H | H | trifluoromethyl | H | H | H | rel-(1R,2S) | 3.44^{[b]} |
| 497 | 2-iodophenyl | cyclobutyl | O | H | H | H | H | trifluoromethyl | H | H | H | rel-(1R,2S) | 3.61^{[b]} |
| 498 | 2-chlorophenyl | cyclobutyl | O | H | H | H | H | trifluoromethyl | H | H | H | rel-(1R,2S) | 3.42^{[b]} |
| 499 | 2-(trifluoromethyl)pyridin-3-yl | cyclopropyl | O | Cl | H | H | H | H | H | H | H | rel-(1R,2S) | 2.62^{[b]} |
| 500 | 2-(trifluoromethyl)pyridin-3-yl | cyclopropyl | O | trifluoromethyl | H | H | H | H | H | H | H | rel-(1R,2S) | 2.79^{[b]} |
| 501 | 2-bromophenyl | cyclobutyl | O | H | H | F | H | trifluoromethyl | H | H | H | rel-(1R,2S) | 3.64^{[b]} |
| 502 | 2-iodophenyl | cyclobutyl | O | H | H | F | H | trifluoromethyl | H | H | H | rel-(1R,2S) | 3.76^{[b]} |
| 503 | 2-iodophenyl | cyclobutyl | O | H | H | F | H | trifluoromethyl | H | H | H | rel-(1R,2R) | 3.50^{[b]} |
| 504 | phenyl | cyclobutyl | O | H | H | F | H | trifluoromethyl | H | H | H | rel-(1R,2S) | 3.42^{[b]} |
| 505 | phenyl | cyclobutyl | O | H | H | F | H | trifluoromethyl | H | H | H | rel-(1R,2R) | 2.97^{[b]} |
| 506 | 2,4-dichlorophenyl | cyclobutyl | O | H | H | F | H | trifluoromethyl | H | H | H | rel-(1R,2R) | 3.85^{[b]} |
| 507 | 4-chlorophenyl | cyclobutyl | O | H | H | F | H | trifluoromethyl | H | H | H | rel-(1R,2S) | 3.94^{[b]} |
| 508 | 4-chlorophenyl | cyclobutyl | O | H | H | F | H | trifluoromethyl | H | H | H | rel-(1R,2R) | 3.62^{[b]} |
| 509 | 2-chlorophenyl | cyclobutyl | O | H | H | F | H | trifluoromethyl | H | H | H | rel-(1R,2R) | 3.34^{[b]} |
| 510 | 2,4-dichlorophenyl | cyclobutyl | O | H | H | F | H | trifluoromethyl | H | H | H | rel-(1R,2S) | 4.08^{[b]} |
| 511 | 3-chlorophenyl | cyclobutyl | O | H | H | F | H | trifluoromethyl | H | H | H | rel-(1R,2R) | 3.65^{[b]} |
| 512 | 2-chlorophenyl | cyclobutyl | O | H | H | F | H | trifluoromethyl | H | H | H | rel-(1R,2S) | 3.56^{[b]} |
| 513 | 2-(trifluoromethyl)phenyl | cyclobutyl | O | F | H | H | H | F | H | H | H | rel-(1R,2R) | 3.15^{[b]} |
| 514 | phenyl | cyclobutyl | O | F | H | H | H | F | H | H | H | rel-(1R,2R) | 2.75^{[b]} |
| 515 | 2-chloropyridin-3-yl | cyclobutyl | O | H | H | F | H | trifluoromethyl | H | H | H | rel-(1R,2R) | 2.73^{[b]} |
| 516 | 3-chloropyrazin-2-yl | cyclobutyl | O | H | H | F | H | trifluoromethyl | H | H | H | rel-(1R,2R) | 2.89^{[b]} |
| 517 | 3-chlorophenyl | cyclobutyl | O | H | F | H | H | trifluoromethyl | H | H | H | rel-(1R,2R) | 3.65^{[b]} |
| 518 | 3-chloropyridin-2-yl | cyclobutyl | O | H | F | H | H | trifluoromethyl | H | H | H | rel-(1R,2R) | 3.08^{[b]} |
| 519 | 3-iodo-2-thienyl | cyclobutyl | O | trifluoromethyl | H | F | H | H | H | H | H | rel-(1R,2S) | 4.09^{[b]} |
| 520 | 3-iodo-2-thienyl | cyclobutyl | O | trifluoromethyl | H | F | H | H | H | H | H | rel-(1R,2R) | 3.82^{[b]} |
| 521 | 2-nitrophenyl | cyclobutyl | O | H | H | F | H | trifluoromethyl | H | H | H | rel-(1R,2S) | 3.10^{[b]} |
| 522 | 2-nitrophenyl | cyclobutyl | O | H | H | F | H | trifluoromethyl | H | H | H | rel-(1R,2R) | 3.04^{[b]} |
| 523 | pyridin-3-yl | cyclobutyl | O | H | H | F | H | trifluoromethyl | H | H | H | rel-(1R,2R) | 2.18^{[b]} |
| 524 | 2-chloropyridin-3-yl | cyclobutyl | O | H | H | F | H | trifluoromethyl | H | H | H | rel-(1R,2S) | 2.93^{[b]} |
| 525 | 3-chloropyrazin-2-yl | cyclobutyl | O | H | H | F | H | trifluoromethyl | H | H | H | rel-(1R,2S) | 3.16^{[b]} |
| 526 | pyridin-3-yl | cyclobutyl | O | H | H | F | H | trifluoromethyl | H | H | H | rel-(1R,2S) | 2.41^{[b]} |
| 527 | 4-chlorophenyl | cyclobutyl | O | H | F | H | H | trifluoromethyl | H | H | H | rel-(1R,2R) | 3.63^{[b]} |
| 528 | 2-chlorophenyl | cyclobutyl | O | H | F | H | H | trifluoromethyl | H | H | H | rel-(1R,2R) | 3.34^{[b]} |
| 529 | 3-chloro-5-(trifluoromethyl)pyridin-2-yl | cyclobutyl | O | H | F | H | H | trifluoromethyl | H | H | H | rel-(1R,2R) | 3.91^{[b]} |
| 530 | 2-methyl-4-(trifluoromethyl)-1,3-thiazol-5-yl | cyclobutyl | O | trifluoromethyl | H | F | H | H | H | H | H | rel-(1R,2S) | 3.60^{[b]} |
| 531 | 2-methyl-4-(trifluoromethyl)-1,3-thiazol-5-yl | cyclobutyl | O | trifluoromethyl | H | F | H | H | H | H | H | rel-(1R,2R) | 3.35^{[b]} |
| 532 | 3-(trifluoromethyl)pyrazin-2-yl | cyclopropyl | O | H | H | Cl | H | H | H | H | H | rel-(1R,2R) | 2.65^{[c]}; |
| | | | | | | | | | | | | | 2.68^{[b]} |
| 533 | 2-chloropyridin-3-yl | cyclobutyl | O | H | H | H | H | Cl | H | H | H | rel-(1R,2S) | 3.65^{[b]} |
| 534 | 2-chloropyridin-3-yl | cyclobutyl | O | H | H | H | H | Cl | H | H | H | rel-(1R,2R) | 2.40^{[b]} |
| 535 | 2,4-dichlorophenyl | cyclobutyl | O | F | H | H | H | trifluoromethyl | H | H | H | rel-(1R,2R) | 3.93^{[b]} |
| 536 | 4-chlorophenyl | cyclobutyl | O | F | H | H | H | trifluoromethyl | H | H | H | rel-(1R,2R) | 3.72^{[b]} |
| 537 | 3-chloropyridin-2-yl | cyclobutyl | O | F | H | H | H | trifluoromethyl | H | H | H | rel-(1R,2R) | 2.92^{[b]} |
| 538 | 2-nitrophenyl | cyclobutyl | O | H | H | H | H | Cl | H | H | H | rel-(1R,2R) | 2.71^{[b]} |
| 539 | pyridin-3-yl | cyclobutyl | O | H | H | H | H | Cl | H | H | H | rel-(1R,2S) | 2.06^{[b]} |
| 540 | 2-nitrophenyl | cyclobutyl | O | H | F | H | H | trifluoromethyl | H | H | H | rel-(1R,2R) | 3.07^{[b]} |
| 541 | 2-iodophenyl | cyclobutyl | O | H | F | H | H | trifluoromethyl | H | H | H | rel-(1R,2R) | 3.52^{[b]} |
| 542 | 2-iodophenyl | cyclobutyl | O | F | H | H | H | trifluoromethyl | H | H | H | rel-(1R,2R) | 3.96^{[b]} |
| 543 | 3-chloro-5-(trifluoromethyl)pyridin-2-yl | cyclobutyl | O | F | H | H | H | trifluoromethyl | H | H | H | rel-(1R,2R) | 4.13^{[b]} |
| 544 | 3-iodo-2-thienyl | cyclobutyl | O | Cl | H | H | H | H | H | H | H | rel-(1R,2S) | 3.86^{[b]} |
| 545 | 2-methyl-4-(trifluoromethyl)-1,3-thiazol-5-yl | cyclobutyl | O | Cl | H | H | H | H | H | H | H | rel-(1R,2R) | 3.15^{[b]} |
| 546 | 3-iodo-2-thienyl | cyclobutyl | O | H | H | H | H | Cl | H | H | H | rel-(1R,2R) | 3.72^{[b]} |
| 547 | 2-nitrophenyl | cyclobutyl | O | H | H | H | H | Cl | H | H | H | rel-(1R,2S) | 2.92^{[b]} |
| 548 | 3-chloropyrazin-2-yl | cyclobutyl | O | H | H | H | H | Cl | H | H | H | rel-(1R,2S) | 2.81^{[b]} |
| 549 | 2,6-difluorophenyl | cyclobutyl | O | H | F | H | H | trifluoromethyl | H | H | H | rel-(1R,2R) | 3.22^{[b]} |
| 550 | phenyl | cyclobutyl | O | H | F | H | H | trifluoromethyl | H | H | H | rel-(1R,2R) | 3.15^{[b]} |
| 551 | phenyl | cyclobutyl | O | F | H | H | H | trifluoromethyl | H | H | H | rel-(1R,2R) | 3.23^{[b]} |
| 552 | 3-chlorophenyl | cyclobutyl | O | F | H | H | H | trifluoromethyl | H | H | H | rel-(1R,2R) | 4.26^{[b]} |
| 553 | 2-chlorophenyl | cyclobutyl | O | F | H | H | H | trifluoromethyl | H | H | H | rel-(1R,2R) | 3.42^{[b]} |
| 554 | 2-methyl-4-(trifluoromethyl)-1,3-thiazol-5-yl | cyclobutyl | O | Cl | H | H | H | H | H | H | H | rel-(1R,2S) | 3.34^{[b]} |
| 555 | pyridin-3-yl | cyclobutyl | O | H | H | H | H | Cl | H | H | H | rel-(1R,2R) | 1.18^{[b]} |
| 556 | 3-(difluoromethyl)-1-methyl-1H-pyrazol-4-yl | cyclobutyl | O | trifluoromethyl | H | H | F | H | H | H | H | rel-(1R,2R) | 2.85^{[b]} |
| 557 | 2,4-dichlorophenyl | cyclobutyl | O | H | F | H | H | trifluoromethyl | H | H | H | rel-(1R,2R) | 3.85^{[b]} |
| 558 | 2-nitrophenyl | cyclopropyl | O | H | H | Cl | H | Cl | H | H | H | rel-(1R,2R) | 2.94^{[b]} |
| 559 | 3-chloropyrazin-2-yl | cyclobutyl | O | H | F | H | H | trifluoromethyl | H | H | H | rel-(1R,2R) | 2.88^{[b]} |
| 560 | 2-bromophenyl | cyclobutyl | O | H | F | H | H | trifluoromethyl | H | H | H | rel-(1R,2R) | 3.38^{[b]} |
| 561 | 2-nitrophenyl | cyclobutyl | O | F | H | H | H | trifluoromethyl | H | H | H | rel-(1R,2R) | 3.03^{[b]} |
| 562 | pyridin-3-yl | cyclobutyl | O | F | H | H | H | trifluoromethyl | H | H | H | rel-(1R,2R) | 2.16^{[b]} |
| 563 | 2-bromophenyl | cyclobutyl | O | F | H | H | H | trifluoromethyl | H | H | H | rel-(1R,2R) | 3.46^{[b]} |
| 564 | 2,6-difluorophenyl | cyclobutyl | O | F | H | H | H | trifluoromethyl | H | H | H | rel-(1R,2R) | 3.25^{[b]} |
| 565 | 2-(trifluoromethyl)pyridin-3-yl | cyclopropyl | O | Cl | H | Cl | H | H | H | H | H | rel-(1R,2R) | 2.85^{[b]} |
| 566 | 3-(trifluoromethyl)pyrazin-2-yl | cyclopropyl | O | Cl | H | Cl | H | H | H | H | H | rel-(1R,2R) | 3.11^{[b]} |
| 567 | 3-chloropyrazin-2-yl | cyclopropyl | O | Cl | H | Cl | H | H | H | H | H | rel-(1R,2R) | 2.75^{[b]} |
| 568 | 2-methyl-4-(trifluoromethyl)-1,3-thiazol-5-yl | cyclopropyl | O | Cl | H | Cl | H | H | H | H | H | rel-(1R,2R) | 3.29^{[b]} |
| 569 | 2-methyl-4-(trifluoromethyl)-1,3-thiazol-5-yl | cyclobutyl | O | trifluoromethyl | H | H | F | H | H | H | H | rel-(1R,2R) | 3.40^{[b]} |
| 570 | 3-iodo-2-thienyl | cyclobutyl | O | trifluoromethyl | H | H | F | H | H | H | H | rel-(1R,2R) | 3.79^{[b]} |
| 571 | 2-chloropyridin-3-yl | cyclobutyl | O | H | F | H | H | trifluoromethyl | H | H | H | rel-(1R,2R) | 2.75^{[b]} |
| 572 | 2-(trifluoromethyl)pyridin-3-yl | cyclobutyl | O | H | F | H | H | trifluoromethyl | H | H | H | rel-(1R,2R) | 3.06^{[b]} |
| 573 | 2-methyl-4-(trifluoromethyl)-1,3-thiazol-5-yl | cyclobutyl | O | trifluoromethyl | H | H | H | F | H | H | H | rel-(1R,2R) | 3.47^{[b]} |
| 574 | 3-(difluoromethyl)-1-methyl-1H-pyrazol-4-yl | cyclobutyl | O | trifluoromethyl | H | H | H | F | H | H | H | rel-(1R,2R) | 2.91^{[b]} |
| 575 | 2-chloropyridin-3-yl | cyclobutyl | O | trifluoromethyl | H | H | H | F | H | H | H | rel-(1R,2R) | 2.83^{[b]} |
| 576 | 3-chloropyrazin-2-yl | cyclobutyl | O | trifluoromethyl | H | H | H | F | H | H | H | rel-(1R,2R) | 3.02^{[b]} |
| 577 | 2-(trifluoromethyl)pyridin-3-yl | cyclobutyl | O | trifluoromethyl | H | H | H | F | H | H | H | rel-(1R,2R) | 3.81^{[b]} |
| 578 | 2-iodophenyl | cyclopropyl | O | H | H | Cl | H | Cl | H | H | H | rel-(1R,2R) | 3.43^{[b]} |
| 579 | 2-bromophenyl | cyclopropyl | O | H | H | Cl | H | Cl | H | H | H | rel-(1R,2R) | 3.32^{[b]} |
| 580 | 2-chlorophenyl | cyclopropyl | O | H | H | Cl | H | Cl | H | H | H | rel-(1R,2R) | 3.27^{[b]} |
| 581 | 2,6-difluorophenyl | cyclopropyl | O | H | H | Cl | H | Cl | H | H | H | rel-(1R,2R) | 3.44^{[b]} |
| 582 | 3-iodo-2-thienyl | cyclopropyl | O | H | H | Cl | H | Cl | H | H | H | rel-(1R,2R) | 3.78^{[b]} |
| 583 | pyridin-3-yl | cyclopropyl | O | H | H | Cl | H | Cl | H | H | H | rel-(1R,2R) | 2.06^{[b]} |
| 584 | 3-iodo-2-thienyl | cyclobutyl | O | trifluoromethyl | H | H | H | F | H | H | H | rel-(1R,2R) | 4.00^{[b]} |
| 585 | 2-(trifluoromethyl)phenyl | cyclobutyl | O | F | H | H | H | trifluoromethyl | H | H | H | rel-(1R,2R) | 3.60^{[b]} |
| 586 | 2-(trifluoromethyl)phenyl | cyclobutyl | O | H | F | H | H | trifluoromethyl | H | H | H | rel-(1R,2R) | 3.53^{[b]} |
| 587 | pyridin-3-yl | cyclobutyl | O | H | F | H | H | trifluoromethyl | H | H | H | rel-(1R,2R) | 2.22^{[b]} |
| 588 | 2-(trifluoromethyl)phenyl | cyclobutyl | O | H | H | H | F | trifluoromethyl | H | H | H | rel-(1R,2S) | 3.63^{[b]} |
| 589 | 2-(trifluoromethyl)phenyl | cyclobutyl | O | H | H | H | F | trifluoromethyl | H | H | H | rel-(1R,2R) | 3.38^{[b]} |
| 590 | 2-iodophenyl | cyclobutyl | O | H | H | H | F | trifluoromethyl | H | H | H | rel-(1R,2S) | 3.68^{[b]} |
| 591 | phenyl | cyclobutyl | O | H | H | H | F | trifluoromethyl | H | H | H | rel-(1R,2S) | 3.35^{[b]} |
| 592 | phenyl | cyclobutyl | O | H | H | H | F | trifluoromethyl | H | H | H | rel-(1R,2R) | 3.01^{[b]} |
| 593 | 2,4-dichlorophenyl | cyclobutyl | O | H | H | H | F | trifluoromethyl | H | H | H | rel-(1R,2S) | 3.50^{[b]} |
| 594 | 2,4-dichlorophenyl | cyclobutyl | O | H | H | H | F | trifluoromethyl | H | H | H | rel-(1R,2R) | 3.73^{[b]} |
| 595 | 4-chlorophenyl | cyclobutyl | O | H | H | H | F | trifluoromethyl | H | H | H | rel-(1R,2R) | 3.47^{[b]} |
| 596 | 3-chlorophenyl | cyclobutyl | O | H | H | H | F | trifluoromethyl | H | H | H | rel-(1R,2S) | 3.92^{[b]} |
| 597 | 3-chlorophenyl | cyclobutyl | O | H | H | H | F | trifluoromethyl | H | H | H | rel-(1R,2R) | 3.50^{[b]} |
| 598 | 2-chlorophenyl | cyclobutyl | O | H | H | H | F | trifluoromethyl | H | H | H | rel-(1R,2S) | 3.50^{[b]} |
| 599 | 2-iodophenyl | cyclobutyl | O | H | H | H | F | trifluoromethyl | H | H | H | rel-(1R,2R) | 3.39^{[b]} |
| 600 | 4-chlorophenyl | cyclobutyl | O | H | H | H | F | trifluoromethyl | H | H | H | rel-(1R,2S) | 3.88^{[b]} |
| 601 | 2-chlorophenyl | cyclobutyl | O | H | H | H | F | trifluoromethyl | H | H | H | rel-(1R,2R) | 3.23^{[b]} |
| 602 | 3-chloro-5-(trifluoromethyl)pyridin-2-yl | cyclobutyl | O | H | H | H | F | trifluoromethyl | H | H | H | rel-(1R,2S) | 4.10^{[b]} |
| 603 | 3-chloro-5-(trifluoromethyl)pyridin-2-yl | cyclobutyl | O | H | H | H | F | trifluoromethyl | H | H | H | rel-(1R,2R) | 3.81^{[b]} |
| 604 | 3-(trifluoromethyl)pyrazin-2-yl | cyclobutyl | O | trifluoromethyl | H | H | H | H | H | H | H | | 3.10^{[c]}; |
| | | | | | | | | | | | | | 3.18^{[b]} |
| 605 | 3-bromopyridin-2-yl | cyclobutyl | O | trifluoromethyl | H | H | H | H | H | H | H | | 3.10^{[c]}; |
| | | | | | | | | | | | | | 3.17^{[b]} |
| 606 | 2-methyl-4,4-dioxido-5,6-dihydro-1,4-oxathiin-3-yl | cyclobutyl | O | trifluoromethyl | H | H | H | H | H | H | H | | 2.45^{[c]}; |
| | | | | | | | | | | | | | 2.49^{[b]} |
| 608 | 3-bromopyridin-2-yl | cyclopropyl | O | H | H | Cl | H | Cl | H | H | H | rel-(1R,2R) | 2.99^{[c]}; |
| | | | | | | | | | | | | | 2.98^{[b]} |
| 609 | 2-(methylsulfonyl)phenyl | cyclopropyl | O | H | H | Cl | H | Cl | H | H | H | rel-(1R,2R) | 2.61^{[c]}; |
| | | | | | | | | | | | | | 2.62^{[b]} |
| 610 | 4-(trifluoromethyl)pyrimidin-5-yl | cyclopropyl | O | H | H | Cl | H | Cl | H | H | H | rel-(1R,2R) | 2.87^{[c]}; |
| | | | | | | | | | | | | | 2.89^{[b]} |
| 611 | 2-methyl-3-furyl | cyclopropyl | O | Cl | H | Cl | H | H | H | H | H | rel-(1R,2R) | 2.89^{[c]}; |
| | | | | | | | | | | | | | 3.02^{[b]} |
| 612 | 5-(difluoromethyl)-1-methyl-1H-pyrazol-4-yl | cyclopropyl | O | Cl | H | Cl | H | H | H | H | H | rel-(1R,2R) | 2.94^{[c]}; |
| | | | | | | | | | | | | | 2.99^{[b]} |
| 613 | 2,4-dichlorophenyl | cyclopropyl | O | H | H | Cl | H | Cl | H | H | H | rel-(1R,2R) | 3.76^{[c]}; |
| | | | | | | | | | | | | | 3.82^{[b]} |
| 614 | 2-methyl-4,4-dioxido-5,6-dihydro-1,4-oxathiin-3-yl | cyclopropyl | O | H | H | Cl | H | Cl | H | H | H | rel-(1R,2R) | 2.33^{[c]}; |
| | | | | | | | | | | | | | 2.40^{[b]} |
| 615 | 2-methyl-5,6-dihydro-1,4-oxathiin-3-yl | cyclopropyl | O | H | H | Cl | H | Cl | H | H | H | rel-(1R,2R) | 3.01^{[c]}; |
| | | | | | | | | | | | | | 3.06^{[b]} |
| 616 | 3-chloropyridin-2-yl | cyclobutyl | O | H | H | H | F | trifluoromethyl | H | H | H | rel-(1R,2S) | 3.30^{[b]} |
| 617 | 3-chloropyridin-2-yl | cyclobutyl | O | H | H | H | F | trifluoromethyl | H | H | H | rel-(1R,2R) | 2.97^{[b]} |
| 618 | pyridin-3-yl | cyclobutyl | O | H | H | H | H | trifluoromethyl | H | H | H | rel-(1R,2S) | 2.26^{[b]} |
| 619 | pyridin-3-yl | cyclobutyl | O | H | H | H | H | trifluoromethyl | H | H | H | rel-(1R,2R) | 2.06^{[b]} |
| 620 | 2-chloropyridin-3-yl | cyclobutyl | O | H | H | H | H | trifluoromethyl | H | H | H | rel-(1R,2S) | 2.79^{[b]} |
| 621 | 2-chloropyridin-3-yl | cyclobutyl | O | H | H | H | H | trifluoromethyl | H | H | H | rel-(1R,2R) | 2.64^{[b]} |
| 622 | 3-chloropyrazin-2-yl | cyclobutyl | O | H | H | H | H | trifluoromethyl | H | H | H | rel-(1R,2S) | 2.98^{[b]} |
| 623 | 3-chloropyrazin-2-yl | cyclobutyl | O | H | H | H | H | trifluoromethyl | H | H | H | rel-(1R,2R) | 2.79^{[b]} |
| 624 | 3-bromopyridin-2-yl | cyclobutyl | O | trifluoromethyl | H | H | H | H | H | H | H | rel-(1R,2S) | 3.27^{[c]}; |
| | | | | | | | | | | | | | 3.34^{[b]} |
| 625 | 2-methyl-4-(trifluoromethyl)-1,3-thiazol-5-yl | cyclobutyl | O | trifluoromethyl | F | H | H | H | H | H | H | rel-(1R,2S) | 3.56^{[b]} |
| 626 | 2-methyl-4-(trifluoromethyl)-1,3-thiazol-5-yl | cyclobutyl | O | trifluoromethyl | F | H | H | H | H | H | H | rel-(1R,2R) | 3.28^{[b]} |
| 627 | 3-iodo-2-thienyl | cyclobutyl | O | trifluoromethyl | F | H | H | H | H | H | H | rel-(1R,2S) | 4.02^{[b]} |
| 628 | 3-iodo-2-thienyl | cyclobutyl | O | trifluoromethyl | F | H | H | H | H | H | H | rel-(1R,2R) | 3.65^{[b]} |
| 629 | 2-nitrophenyl | cyclobutyl | O | H | H | H | F | trifluoromethyl | H | H | H | rel-(1R,2S) | 3.18^{[b]} |
| 630 | 2-nitrophenyl | cyclobutyl | O | H | H | H | F | trifluoromethyl | H | H | H | rel-(1R,2R) | 2.94^{[b]} |
| 631 | pyridin-3-yl | cyclobutyl | O | H | H | H | F | trifluoromethyl | H | H | H | rel-(1R,2R) | 2.12^{[b]} |
| 632 | 2-bromophenyl | cyclobutyl | O | H | H | H | F | trifluoromethyl | H | H | H | rel-(1R,2S) | 3.57^{[b]} |
| 633 | 2-methyl-4-(trifluoromethyl)-1,3-thiazol-5-yl | cyclobutyl | O | trifluoromethyl | H | H | H | H | H | H | H | rel-(1R,2S) | 3.49^{[b]} |
| 634 | 2-methyl-4-(trifluoromethyl)-1,3-thiazol-5-yl | cyclobutyl | O | trifluoromethyl | H | H | H | H | H | H | H | rel-(1R,2R) | 3.34^{[b]} |
| 635 | 3-iodo-2-thienyl | cyclobutyl | O | trifluoromethyl | H | H | H | H | H | H | H | rel-(1R,2S) | 3.99^{[b]} |
| 636 | 3-iodo-2-thienyl | cyclobutyl | O | trifluoromethyl | H | H | H | H | H | H | H | rel-(1R,2R) | 3.83^{[b]} |
| 637 | 2-nitrophenyl | cyclobutyl | O | H | H | H | H | trifluoromethyl | H | H | H | rel-(1R,2S) | 3.13^{[b]} |
| 638 | 2-nitrophenyl | cyclobutyl | O | H | H | H | H | trifluoromethyl | H | H | H | rel-(1R,2R) | 2.93^{[b]} |
| 639 | 2,6-difluorophenyl | cyclobutyl | O | H | H | H | F | trifluoromethyl | H | H | H | rel-(1R,2R) | 3.08^{[b]} |
| 640 | 2,6-difluorophenyl | cyclobutyl | O | H | H | H | F | trifluoromethyl | H | H | H | rel-(1R,2S) | 3.36^{[b]} |
| 641 | 2-(trifluoromethyl)pyridin-3-yl | cyclobutyl | O | H | H | H | F | trifluoromethyl | H | H | H | rel-(1R,2R) | 2.97^{[b]} |
| 642 | 2-(trifluoromethyl)pyridin-3-yl | cyclobutyl | O | H | H | H | F | trifluoromethyl | H | H | H | rel-(1R,2S) | 3.15^{[b]} |
| 643 | 3-(difluoromethyl)-1-methyl-1H-pyrazol-4-yl | cyclobutyl | O | H | H | H | F | trifluoromethyl | H | H | H | rel-(1R,2R) | 2.75^{[b]} |
| 644 | 3-(difluoromethyl)-1-methyl-1H-pyrazol-4-yl | cyclobutyl | O | H | H | H | F | trifluoromethyl | H | H | H | rel-(1R,2S) | 2.99^{[b]} |
| 645 | 3-chloropyrazin-2-yl | cyclobutyl | O | H | H | H | F | trifluoromethyl | H | H | H | rel-(1R,2R) | 2.78^{[b]} |
| 646 | 3-chloropyrazin-2-yl | cyclobutyl | O | H | H | H | F | trifluoromethyl | H | H | H | rel-(1R,2S) | 3.10^{[b]} |
| 647 | 2-chloropyridin-3-yl | cyclobutyl | O | H | H | H | F | trifluoromethyl | H | H | H | rel-(1R,2R) | 2.66^{[b]} |
| 648 | 2-chloropyridin-3-yl | cyclobutyl | O | H | H | H | F | trifluoromethyl | H | H | H | rel-(1R,2S) | 2.86^{[b]} |
| 649 | 3-chloropyridin-2-yl | methylene | O | H | H | Cl | H | Cl | H | H | H | rel-(1R,2R) | 2.91^{[c]}; |
| | | | | | | | | | | | | | 2.93^{[b]} |
| 650 | 2-cyanophenyl | methylene | O | H | H | Cl | H | Cl | H | H | H | rel-(1R,2R) | 2.70^{[c]}; |
| | | | | | | | | | | | | | 2.72^{[b]} |
| 654 | 2-bromophenyl | cyclobutyl | O | H | H | H | F | trifluoromethyl | H | H | H | rel-(1R,2R) | 3.30^{[b]} |
| 655 | 2-(trifluoromethyl)phenyl | cyclobutyl | O | H | H | Br | H | trifluoromethyl | H | H | H | rel-(1R,2S) | 4.19^{[c]}; |
| | | | | | | | | | | | | | 4.26^{[b]} |
| 656 | 2-(trifluoromethyl)phenyl | cyclobutyl | O | H | H | Br | H | trifluoromethyl | H | H | H | rel-(1R,2R) | 3.94^{[c]}; |
| | | | | | | | | | | | | | 4.02^{[b]} |
| 657 | 2-(trifluoromethyl)pyridin-3-yl | cyclobutyl | O | H | H | Br | H | trifluoromethyl | H | H | H | rel-(1R,2R) | 3.42^{[c]}; |
| | | | | | | | | | | | | | 3.52^{[b]} |
| 658 | 2-(trifluoromethyl)pyridin-3-yl | cyclobutyl | O | H | H | Br | H | trifluoromethyl | H | H | H | rel-(1R,2S) | 3.66^{[c]}; |
| | | | | | | | | | | | | | 3.76^{[b]} |
| 659 | 3-(trifluoromethyl)pyrazin-2-yl | cyclobutyl | O | H | H | Br | H | trifluoromethyl | H | H | H | rel-(1R,2R) | 3.62^{[c]}; |
| | | | | | | | | | | | | | 3.72^{[b]} |
| 660 | 3-(trifluoromethyl)pyrazin-2-yl | cyclobutyl | O | H | H | Br | H | trifluoromethyl | H | H | H | rel-(1R,2S) | 3.87^{[c]}; |
| | | | | | | | | | | | | | 3.97^{[b]} |
| 661 | 2-nitrophenyl | cyclobutyl | O | H | H | Cl | H | Cl | H | H | H | rel-(1R,2R) | 3.19^{[b]} |
| 662 | pyridin-3-yl | cyclobutyl | O | H | H | Cl | H | Cl | H | H | H | rel-(1R,2S) | 2.58^{[b]} |
| 663 | 2-chloropyridin-3-yl | cyclobutyl | O | H | H | Cl | H | Cl | H | H | H | rel-(1R,2R) | 2.84^{[b]} |
| 664 | pyridin-3-yl | cyclobutyl | O | H | H | H | F | trifluoromethyl | H | H | H | rel-(1R,2S) | 2.38^{[b]} |
| 665 | 2,4-dichlorophenyl | cyclobutyl | O | H | H | Cl | H | trifluoromethyl | H | H | H | rel-(1R,2S) | 4.55^{[b]} |
| 666 | 2-chlorophenyl | cyclobutyl | O | H | H | Cl | H | trifluoromethyl | H | H | H | rel-(1R,2R) | 3.75^{[b]} |
| 667 | 3-chloro-5-(trifluoromethyl)pyridin-2-yl | cyclobutyl | O | H | H | Cl | H | trifluoromethyl | H | H | H | rel-(1R,2R) | 4.33^{[b]} |
| 668 | 2-methyl-4-(trifluoromethyl)-1,3-thiazol-5-yl | cyclobutyl | O | Cl | H | Cl | H | H | H | H | H | rel-(1R,2S) | 3.91^{[b]} |
| 669 | 2-methyl-4-(trifluoromethyl)-1,3-thiazol-5-yl | cyclobutyl | O | Cl | H | Cl | H | H | H | H | H | rel-(1R,2R) | 3.58^{[b]} |
| 670 | 3-iodo-2-thienyl | cyclobutyl | O | Cl | H | Cl | H | H | H | H | H | rel-(1R,2S) | 4.51^{[b]} |
| 671 | 3-iodo-2-thienyl | cyclobutyl | O | Cl | H | Cl | H | H | H | H | H | rel-(1R,2R) | 4.20^{[b]} |
| 672 | 2-nitrophenyl | cyclobutyl | O | H | H | Cl | H | Cl | H | H | H | rel-(1R,2S) | 3.47^{[b]} |
| 673 | pyridin-3-yl | cyclobutyl | O | H | H | Cl | H | Cl | H | H | H | rel-(1R,2R) | 2.23^{[b]} |
| 674 | 2-chloropyridin-3-yl | cyclobutyl | O | H | H | Cl | H | Cl | H | H | H | rel-(1R,2S) | 3.16^{[b]} |
| 675 | 3-chloropyrazin-2-yl | cyclobutyl | O | H | H | Cl | H | Cl | H | H | H | rel-(1R,2S) | 3.43^{[b]} |
| 676 | 3-chloropyrazin-2-yl | cyclobutyl | O | H | H | Cl | H | Cl | H | H | H | rel-(1R,2R) | 3.05^{[b]} |
| 677 | 2-iodophenyl | cyclobutyl | O | H | H | Cl | H | trifluoromethyl | H | H | H | rel-(1R,2S) | 4.20^{[b]} |
| 678 | 2-iodophenyl | cyclobutyl | O | H | H | Cl | H | trifluoromethyl | H | H | H | rel-(1R,2R) | 3.90^{[b]} |
| 679 | phenyl | cyclobutyl | O | H | H | Cl | H | trifluoromethyl | H | H | H | rel-(1R,2S) | 3.87^{[b]} |
| 680 | phenyl | cyclobutyl | O | H | H | Cl | H | trifluoromethyl | H | H | H | rel-(1R,2R) | 3.51^{[b]} |
| 684 | 3-(trifluoromethyl)pyrazin-2-yl | cyclobutyl | O | trifluoromethyl | H | H | H | H | H | H | H | rel-(1R,2R) | 3.10^{[c]}; |
| | | | | | | | | | | | | | 3.15^{[b]} |
| 687 | 3-(trifluoromethyl)pyrazin-2-yl | cyclobutyl | O | trifluoromethyl | H | H | H | H | H | H | H | rel-(1R,2S) | 3.25^{[c]}; |
| | | | | | | | | | | | | | 3.31^{[b]} |
| 691 | 3-bromopyridin-2-yl | cyclobutyl | O | trifluoromethyl | H | H | H | H | H | H | H | rel-(1R,2S) | 3.27^{[c]}; |
| | | | | | | | | | | | | | 3.34^{[b]} |
| 695 | 3-bromopyridin-2-yl | cyclobutyl | O | trifluoromethyl | H | H | H | H | H | H | H | rel-(1R,2R) | 3.10^{[c]}; |
| | | | | | | | | | | | | | 3.17^{[b]} |
| 697 | 2-chlorophenyl | cyclobutyl | O | H | H | Cl | H | trifluoromethyl | H | H | H | rel-(1R,2S) | 4.01^{[b]} |
| 698 | 3-chloropyridin-2-yl | cyclobutyl | O | H | H | Cl | H | trifluoromethyl | H | H | H | rel-(1R,2S) | 3.84^{[b]} |
| 699 | 3-chloropyridin-2-yl | cyclobutyl | O | H | H | Cl | H | trifluoromethyl | H | H | H | rel-(1R,2R) | 3.49^{[b]} |
| 700 | 3-chloro-5-(trifluoromethyl)pyridin-2-yl | cyclobutyl | O | H | H | Cl | H | trifluoromethyl | H | H | H | rel-(1R,2S) | 4.65^{[b]} |
| 701 | 2,6-difluorophenyl | cyclobutyl | O | H | H | Cl | H | trifluoromethyl | H | H | H | rel-(1R,2S) | 3.75^{[b]} |
| 702 | 2,6-difluorophenyl | cyclobutyl | O | H | H | Cl | H | trifluoromethyl | H | H | H | rel-(1R,2R) | 3.52^{[b]} |
| 703 | 2-(trifluoromethyl)phenyl | cyclobutyl | O | H | H | Cl | H | trifluoromethyl | H | H | H | rel-(1R,2S) | 4.10^{[b]} |
| 704 | 2-(trifluoromethyl)phenyl | cyclobutyl | O | H | H | Cl | H | trifluoromethyl | H | H | H | rel-(1R,2R) | 3.84^{[b]} |
| 705 | 2,4-dichlorophenyl | cyclobutyl | O | H | H | Cl | H | trifluoromethyl | H | H | H | rel-(1R,2R) | 4.27^{[b]} |
| 706 | 4-chlorophenyl | cyclobutyl | O | H | H | Cl | H | trifluoromethyl | H | H | H | rel-(1R,2S) | 4.43^{[b]} |
| 707 | 4-chlorophenyl | cyclobutyl | O | H | H | Cl | H | trifluoromethyl | H | H | H | rel-(1R,2R) | 4.04^{[b]} |
| 708 | 3-chlorophenyl | cyclobutyl | O | H | H | Cl | H | trifluoromethyl | H | H | H | rel-(1R,2S) | 4.46^{[b]} |
| 709 | 3-chlorophenyl | cyclobutyl | O | H | H | Cl | H | trifluoromethyl | H | H | H | rel-(1R,2R) | 4.05^{[b]} |
| 710 | 3-iodo-2-thienyl | cyclobutyl | O | trifluoromethyl | H | Cl | H | H | H | H | H | rel-(1R,2S) | 4.55^{[b]} |
| 711 | 3-iodo-2-thienyl | cyclobutyl | O | trifluoromethyl | H | Cl | H | H | H | H | H | rel-(1R,2R) | 4.28^{[b]} |
| 712 | 2-nitrophenyl | cyclobutyl | O | H | H | Cl | H | trifluoromethyl | H | H | H | rel-(1R,2S) | 3.61^{[b]} |
| 713 | 2-nitrophenyl | cyclobutyl | O | H | H | Cl | H | trifluoromethyl | H | H | H | rel-(1R,2R) | 3.36^{[b]} |
| 714 | pyridin-3-yl | cyclobutyl | O | H | H | Cl | H | trifluoromethyl | H | H | H | rel-(1R,2R) | 2.50^{[b]} |
| 715 | 2-chloropyridin-3-yl | cyclobutyl | O | H | H | Cl | H | trifluoromethyl | H | H | H | rel-(1R,2S) | 3.33^{[b]} |
| 716 | 3-chloropyrazin-2-yl | cyclobutyl | O | H | H | Cl | H | trifluoromethyl | H | H | H | rel-(1R,2S) | 3.56^{[b]} |
| 717 | 3-chloropyrazin-2-yl | cyclobutyl | O | H | H | Cl | H | trifluoromethyl | H | H | H | rel-(1R,2R) | 3.24^{[b]} |
| 718 | 3-(difluoromethyl)-1-methyl-1H-pyrazol-4-yl | cyclobutyl | O | trifluoromethyl | H | Cl | H | H | H | H | H | rel-(1R,2S) | 3.41^{[b]} |
| 719 | 3-(difluoromethyl)-1-methyl-1H-pyrazol-4-yl | cyclobutyl | O | trifluoromethyl | H | Cl | H | H | H | H | H | rel-(1R,2R) | 3.17^{[b]} |
| 720 | 2-(trifluoromethyl)pyridin-3-yl | cyclobutyl | O | H | H | Cl | H | trifluoromethyl | H | H | H | rel-(1R,2S) | 3.60^{[b]} |
| 721 | 2-(trifluoromethyl)pyridin-3-yl | cyclobutyl | O | H | H | Cl | H | trifluoromethyl | H | H | H | rel-(1R,2R) | 3.36^{[b]} |
| 722 | 2-bromophenyl | cyclobutyl | O | H | H | Cl | H | trifluoromethyl | H | H | H | rel-(1R,2S) | 4.08^{[b]} |
| 723 | 2-bromophenyl | cyclobutyl | O | H | H | Cl | H | trifluoromethyl | H | H | H | rel-(1R,2R) | 3.80^{[b]} |
| 724 | pyridin-3-yl | cyclobutyl | O | Cl | H | H | H | Cl | H | H | H | rel-(1R,2R) | 2.17^{[b]} |
| 725 | 2-chloropyridin-3-yl | cyclobutyl | O | Cl | H | H | H | Cl | H | H | H | rel-(1R,2R) | 2.79^{[b]} |
| 726 | 3-(difluoromethyl)-1-methyl-1H-pyrazol-4-yl | cyclobutyl | O | Cl | H | H | H | Cl | H | H | H | rel-(1R,2R) | 2.90^{[b]} |
| 727 | 2,6-difluorophenyl | cyclobutyl | O | Cl | H | H | H | Cl | H | H | H | rel-(1R,2R) | 3.33^{[b]} |
| 728 | phenyl | cyclobutyl | O | Cl | H | H | H | Cl | H | H | H | rel-(1R,2R) | 3.30^{[b]} |
| 729 | 3-chlorophenyl | cyclobutyl | O | Cl | H | H | H | Cl | H | H | H | rel-(1R,2R) | 3.87^{[b]} |
| 730 | 2-methyl-4-(trifluoromethyl)-1,3-thiazol-5-yl | cyclobutyl | O | trifluoromethyl | H | Cl | H | H | H | H | H | rel-(1R,2S) | 4.03^{[b]} |
| 731 | 2-methyl-4-(trifluoromethyl)-1,3-thiazol-5-yl | cyclobutyl | O | trifluoromethyl | H | Cl | H | H | H | H | H | rel-(1R,2R) | 3.75^{[b]} |
| 732 | pyridin-3-yl | cyclobutyl | O | trifluoromethyl | H | Cl | H | H | H | H | H | rel-(1R,2S) | 2.74^{[b]} |
| 733 | 2-nitrophenyl | cyclobutyl | O | Cl | H | H | H | Cl | H | H | H | rel-(1R,2R) | 3.08^{[b]} |
| 734 | 3-chloropyrazin-2-yl | cyclobutyl | O | Cl | H | H | H | Cl | H | H | H | rel-(1R,2R) | 3.07^{[b]} |
| 735 | 2-bromophenyl | cyclobutyl | O | Cl | H | H | H | Cl | H | H | H | rel-(1R,2R) | 3.60^{[b]} |
| 736 | 2-(trifluoromethyl)phenyl | cyclobutyl | O | Cl | H | H | H | Cl | H | H | H | rel-(1R,2R) | 3.71^{[b]} |
| 737 | 2-iodophenyl | cyclobutyl | O | Cl | H | H | H | Cl | H | H | H | rel-(1R,2R) | 3.72^{[b]} |
| 738 | 4-chlorophenyl | cyclobutyl | O | Cl | H | H | H | Cl | H | H | H | rel-(1R,2R) | 3.36^{[b]} |
| 739 | 2-chloropyridin-3-yl | cyclobutyl | O | trifluoromethyl | H | Cl | H | H | H | H | H | rel-(1R,2R) | 3.09^{[b]} |
| 740 | 2-methyl-4-(trifluoromethyl)-1,3-thiazol-5-yl | cyclobutyl | O | Cl | H | H | H | Cl | H | H | H | rel-(1R,2R) | 3.60^{[b]} |
| 741 | 2,4-dichlorophenyl | cyclobutyl | O | Cl | H | H | H | Cl | H | H | H | rel-(1R,2R) | 4.10^{[b]} |
| 742 | 2-chlorophenyl | cyclobutyl | O | Cl | H | H | H | Cl | H | H | H | rel-(1R,2R) | 3.56^{[b]} |
| 743 | 3-chloropyridin-2-yl | cyclobutyl | O | Cl | H | H | H | Cl | H | H | H | rel-(1R,2R) | 3.32^{[b]} |
| 744 | 3-chloro-5-(trifluoromethyl)pyridin-2-yl | cyclobutyl | O | Cl | H | H | H | Cl | H | H | H | rel-(1R,2R) | 4.28^{[b]} |
| 745 | 3-iodo-2-thienyl | cyclobutyl | O | Cl | H | H | H | Cl | H | H | H | rel-(1R,2R) | 4.21^{[b]} |
| 746 | 2-(trifluoromethyl)pyridin-3-yl | cyclobutyl | O | Cl | H | H | H | Cl | H | H | H | rel-(1R,2R) | 2.95^{[b]} |
| 747 | 2-methyl-4-(trifluoromethyl)-1,3-thiazol-5-yl | cyclopentyl | O | H | H | trifluoromethyl | H | H | H | H | H | rel-(1R,2R) | 3.71^{[b]} |
| 748 | 3-chloropyrazin-2-yl | cyclopentyl | O | H | H | trifluoromethyl | H | H | H | H | H | rel-(1R,2R) | 3.20^{[b]} |
| 749 | 3-iodo-2-thienyl | cyclopropyl | O | H | H | Cl | H | H | F | H | H | rel-(1R,2S) | 3.15^{[b]} |
| 750 | 2,6-difluorophenyl | cyclopropyl | O | H | H | Cl | H | H | F | H | H | rel-(1R,2S) | 2.69^{[b]} |
| 751 | 2-bromophenyl | cyclopropyl | O | H | H | Cl | H | H | F | H | H | rel-(1R,2S) | 2.84^{[b]} |
| 752 | 2-chlorophenyl | cyclopropyl | O | H | H | Cl | H | H | F | H | H | rel-(1R,2S) | 2.78^{[b]} |
| 753 | 2-iodophenyl | cyclopropyl | O | H | H | Cl | H | H | F | H | H | rel-(1R,2S) | 2.98^{[b]} |
| 754 | 2-(trifluoromethyl)phenyl | cyclopropyl | O | H | H | Cl | H | H | F | H | H | rel-(1R,2R) | 3.24^{[b]} |
| 755 | 2-(trifluoromethyl)phenyl | cyclopropyl | O | H | H | Cl | H | H | F | H | H | rel-(1R,2S) | 3.03^{[b]} |
| 756 | 2,6-difluorophenyl | cyclopropyl | O | H | H | Cl | H | H | F | H | H | rel-(1R,2R) | |

Compounds of formula (Ia)

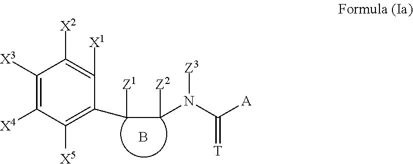

wherein A, B, T, X¹, X², X³, X⁴, X⁵, Z¹, Z² and Z³ have the meanings given in the table. If stereochemistry is known, the relative positions of the ring carbons are named in a way, that the carbon connected to Z² is position 1, the carbon connected to Z¹ is position 2.

### Measurement of log P-Values:

Measurement of log P Values was Performed According EEC Directive 79/831 Annex V.A8 by HPLC (High Performance Liquid

Chromatography) on reversed phase columns with the following methods:
^{[a]} Measurement was done at pH 2.3 with 0.1% phosphoric acid and acetonitrile as eluent with a linear gradient from 10% acetonitrile to 95% acetonitrile.
^{[b]} measurement of LC-MS was done at pH 2.7 with 0.1% formic acid in water and with acetonitrile (contains 0.1% formic acid) as eluent with a linear gradient from 10% acetonitrile to 95% acetonitrile.
^{[c]} Measurement with LC-MS was done at pH 7.8 with 0.001 molar ammonium hydrogen carbonate solution in water as eluent with a linear gradient from 10% acetonitrile to 95% acetonitrile.

Calibration was done with linear alkan-2-ones (with 3 to 16 carbon atoms) with known log P-values (measurement of log P values using retention times with linear interpolation between successive alkanones). Lambda-max-values were determined using UV-spectra from 200 nm to 400 nm and the peak values of the chromatographic signals.

### NMR-Peak Lists

1H-NMR data of selected examples are written in form of 1H-NMR-peak lists. To each signal peak are listed the δ-value in ppm and the signal intensity in round brackets. Between the δ-value—signal intensity pairs are semicolons as delimiters.

The peak list of an example has therefore the form:

δ₁ (intensity₁); δ₂ (intensity₂); . . . ; δᵢ (intensityᵢ); . . . ; δₙ (intensityₙ)

Intensity of sharp signals correlates with the height of the signals in a printed example of a NMR spectrum in cm and shows the real relations of signal intensities. From broad signals several peaks or the middle of the signal and their relative intensity in comparison to the most intensive signal in the spectrum can be shown.

For calibrating chemical shift for 1H spectra, we use tetramethylsilane and/or the chemical shift of the solvent used, especially in the case of spectra measured in DMSO. Therefore in NMR peak lists, tetramethylsilane peak can occur but not necessarily.

The 1H-NMR peak lists are similar to classical 1H-NMR prints and contain therefore usually all peaks, which are listed at classical NMR-interpretation.

Additionally they can show like classical 1H-NMR prints signals of solvents, stereoisomers of the target compounds, which are also object of the invention, and/or peaks of impurities.

To show compound signals in the delta-range of solvents and/or water the usual peaks of solvents, for example peaks of DMSO in DMSO-D₆ and the peak of water are shown in our 1H-NMR peak lists and have usually on average a high intensity.

The peaks of stereoisomers of the target compounds and/or peaks of impurities have usually on average a lower intensity than the peaks of target compounds (for example with a purity >90%).

Such stereoisomers and/or impurities can be typical for the specific preparation process. Therefore their peaks can help to recognize the reproduction of our preparation process via “side-products-fingerprints”.

An expert, who calculates the peaks of the target compounds with known methods (MestreC, ACD-simulation, but also with empirically evaluated expectation values) can isolate the peaks of the target compounds as needed optionally using additional intensity filters. This isolation would be similar to relevant peak picking at classical 1H-NMR interpretation.

Further details of NMR-data description using peak lists are available in the publication “Citation of NMR Peaklist Data within patent applications” (Research Disclosure Database Number 564025).

Example 1: ¹H-NMR(400.0 MHz. DMSO): δ = 7.949(3.9); 7.887(1.5); 7.865(1.5); 7.329(1.1); 7.228(3.3); 7.219(15.2); 7.212(6.3); 7.206(5.8); 7.185(0.9); 7.135(0.5); 7.123(1.2);
7.116(1.3); 7.109(1.3); 7.102(1.4); 7.094(0.9); 7.087(0.7); 7.080(0.4); 4.025(0.3); 4.009(0.7); 3.987(0.8); 3.959(0.5); 3.929(0.3); 3.908(2.1); 3.902(3.9); 3.850(16.0); 3.326(101.8);
3.168(0.4); 2.675(0.6); 2.670(0.8); 2.666(0.6); 2.650(0.6); 2.642(0.7); 2.620(1.0); 2.612(1.1); 2.592(0.7); 2.583(0.7); 2.541(0.6); 2.524(2.0); 2.510(52.5); 2.506(104.1); 2.502(135.1);
2.497(96.8); 2.493(47.0); 2.333(0.6); 2.328(0.8); 2.324(0.6); 1.918(1.0); 1.894(0.9); 1.831(0.8); 1.794(1.7); 1.761(1.7); 1.724(1.0); 1.542(0.5); 1.535(0.4); 1.511(0.9); 1.504(0.8);
1.478(1.0); 1.445(0.7); 1.405(0.9); 1.387(1.1); 1.366(1.6); 1.333(1.2); 1.300(0.7); 0.008(0.3); 0.000(9.4); −0.008(0.4)
Example 2: ¹H-NMR(400.0 MHz. DMSO): δ = 8.316(0.5); 8.076(0.9); 8.054(0.9); 7.957(0.3); 7.932(0.3); 7.268(0.3); 7.239(0.7); 7.220(10.7); 7.209(11.0); 7.203(5.6); 7.196(3.4);
7.193(3.2); 7.187(3.2); 7.183(2.5); 7.166(1.3); 7.148(0.9); 7.136(1.4); 7.125(1.8); 7.115(2.0); 7.104(1.3); 7.093(0.7); 7.076(0.5); 4.365(0.8); 4.184(0.4); 4.162(0.5); 4.157(0.4);
4.136(0.5); 3.943(0.6); 3.933(0.6); 3.902(6.8); 3.324(162.4); 3.226(0.5); 3.210(0.7); 3.201(0.8); 3.182(1.6); 3.167(2.1); 3.162(1.8); 3.147(1.2); 3.136(0.6); 3.130(0.6); 3.118(0.6);
3.111(1.1); 3.099(1.2); 3.093(1.2); 3.081(1.2); 3.075(0.5); 3.063(0.5); 3.026(0.5); 2.994(0.7); 2.954(0.5); 2.888(1.5); 2.764(0.8); 2.675(1.0); 2.671(1.3); 2.666(1.0); 2.559(0.8);
2.550(1.0); 2.541(5.4); 2.524(4.3); 2.511(78.1); 2.506(153.1); 2.502(198.2); 2.497(140.5); 2.493(66.8); 2.442(0.3); 2.337(0.6); 2.333(1.0); 2.328(1.4); 2.324(1.0); 1.932(0.4); 1.926(0.4);
1.902(6.4); 1.873(2.7); 1.855(1.2); 1.840(1.2); 1.806(1.7); 1.770(2.4); 1.738(2.5); 1.719(1.5); 1.702(1.8); 1.686(1.4); 1.667(1.2); 1.652(0.9); 1.635(0.5); 1.615(0.5); 1.600(0.8);
1.579(0.9); 1.564(0.7); 1.544(0.6); 1.529(0.5); 1.499(0.9); 1.468(1.2); 1.429(1.3); 1.403(1.1); 1.396(1.1); 1.388(1.1); 1.364(1.2); 1.357(1.4); 1.330(1.4); 1.316(1.4); 1.294(2.0);
1.285(2.1); 1.272(1.5); 1.255(16.0); 1.196(2.4); 1.187(0.8); 1.178(4.8); 1.159(2.3); 1.152(0.8); 1.091(0.6); 1.053(0.4); 1.039(0.5); 0.849(0.3); 0.793(0.6); 0.769(0.6); 0.684(0.5);
0.422(1.0); 0.332(1.0); 0.300(0.6); 0.283(0.6); 0.008(0.9); 0.000(19.9); −0.009(0.6)
Example 3: ¹H-NMR(400.0 MHz. DMSO): δ = 7.222(7.3); 7.211(12.4); 7.200(1.4); 7.178(1.0); 7.139(0.7); 7.128(1.1); 7.118(1.2); 7.110(0.4); 7.106(0.7); 3.989(0.5); 3.980(0.4);
3.967(0.5); 3.902(1.1); 3.531(9.3); 3.529(9.1); 3.325(68.4); 2.685(0.4); 2.676(0.7); 2.671(0.6); 2.666(0.4); 2.656(0.8); 2.648(0.7); 2.627(0.5); 2.618(0.5); 2.524(1.3); 2.511(27.1);
2.506(53.9); 2.502(70.1); 2.497(50.0); 2.493(23.7); 2.328(0.4); 1.956(0.7); 1.944(0.4); 1.932(0.5); 1.902(16.0); 1.812(0.5); 1.798(0.5); 1.783(1.0); 1.753(1.1); 1.715(0.6); 1.530(0.6);
1.523(0.5); 1.499(0.6); 1.492(0.5); 1.443(0.4); 1.416(0.9); 1.411(0.9); 1.388(1.4); 1.361(0.6); 1.318(0.5); 1.288(0.4); 0.000(5.9)
Example 8: ¹H-NMR(400.0 MHz. DMSO): δ = 9.153(1.2); 9.143(1.2); 7.295(1.3); 7.276(3.4); 7.257(2.6); 7.194(1.3); 7.176(1.8); 7.171(0.6); 7.155(3.3); 7.152(3.4); 7.134(2.6);
3.902(1.0); 3.323(57.5); 2.999(0.3); 2.989(0.6); 2.980(0.8); 2.976(0.8); 2.969(0.7); 2.963(0.6); 2.959(0.6); 2.949(0.3); 2.726(16.0); 2.675(0.4); 2.671(0.5); 2.666(0.3); 2.645(0.5);
2.541(0.4); 2.524(1.5); 2.510(31.1); 2.506(60.7); 2.502(77.8); 2.497(55.4); 2.493(26.4); 2.333(0.3); 2.328(0.5); 2.324(0.4); 2.064(0.5); 2.056(0.6); 2.045(0.9); 2.039(0.8); 2.034(0.6);
2.025(0.6); 2.016(0.6); 1.254(1.6); 1.241(1.2); 1.232(3.5); 1.218(1.6); 1.215(1.5); 0.000(7.8)
Example 9: ¹H-NMR(400.0 MHz. DMSO): δ = 7.543(1.0); 7.534(1.0); 7.247(0.6); 7.243(0.4); 7.233(0.9); 7.227(3.1); 7.218(3.7); 7.212(10.6); 7.197(0.9); 7.161(0.7); 7.155(0.8);
7.146(0.9); 7.139(1.0); 7.131(0.5); 7.124(0.5); 7.118(0.3); 3.902(2.2); 3.780(1.3); 3.734(0.4); 3.639(16.0); 3.323(87.3); 3.125(0.5); 3.118(0.6); 3.114(0.5); 3.106(1.1); 3.095(0.7);
3.088(0.5); 2.675(0.4); 2.671(0.5); 2.666(0.4); 2.524(1.6); 2.510(33.2); 2.506(65.1); 2.501(83.7); 2.497(59.7); 2.493(28.4); 2.328(1.7); 2.306(0.3); 2.289(0.5); 2.271(1.1); 2.258(1.2);
2.249(1.2); 2.232(0.9); 1.845(15.9); 1.290(0.5); 1.275(0.9); 1.271(0.7); 1.269(0.6); 1.254(1.2); 1.246(1.0); 1.233(2.0); 1.216(1.1); 1.201(0.4); 0.000(5.0)
Example 27: ¹H-NMR(400.0 MHz. DMSO): δ = 8.955(3.5); 8.944(3.5); 8.829(7.3); 8.824(7.3); 8.315(0.7); 8.246(5.6); 8.240(5.3); 8.225(6.0); 8.219(5.8); 7.666(7.8); 7.665(7.7);
7.645(7.2); 7.644(7.2); 7.302(3.6); 7.286(4.9); 7.282(10.4); 7.269(2.9); 7.264(7.1); 7.195(3.5); 7.193(3.2); 7.177(16.0); 7.173(11.7); 7.156(11.1); 3.902(2.4); 3.326(385.1); 3.057(1.0);
3.045(2.2); 3.037(3.2); 3.026(3.2); 3.017(2.3); 3.006(1.0); 2.680(0.6); 2.676(1.1); 2.671(1.6); 2.666(1.1); 2.662(0.5); 2.541(1.0); 2.524(4.1); 2.511(93.5); 2.507(187.0); 2.502(242.6);
2.497(171.1); 2.493(79.3); 2.338(0.5); 2.333(1.1); 2.329(1.5); 2.324(1.1); 2.320(0.5); 2.134(1.6); 2.125(1.6); 2.118(2.0); 2.110(3.4); 2.102(1.9); 2.095(1.8); 2.086(1.7); 1.374(1.8);
1.360(3.2); 1.349(3.2); 1.337(3.0); 1.325(2.2); 1.283(2.4); 1.268(3.6); 1.264(3.1); 1.254(2.4); 1.249(3.5); 1.234(1.9); 0.008(0.8); 0.000(24.5); −0.008(0.7)
Example 31: ¹H-NMR(400.0 MHz. DMSO): δ = 8.753(0.4); 8.310(4.2); 7.461(0.5); 7.326(9.5); 7.309(11.0); 7.305(15.9); 7.288(16.0); 7.256(13.4); 7.245(1.7); 7.235(6.1); 7.220(1.0);
7.203(12.0); 7.181(8.5); 7.140(0.6); 7.119(0.5); 3.899(3.1); 3.778(1.0); 3.639(0.4); 3.334(109.6); 3.110(2.1); 3.101(2.0); 3.083(1.8); 2.999(0.4); 2.977(2.3); 2.946(0.8); 2.849(1.5);
2.787(0.6); 2.677(1.7); 2.672(2.2); 2.668(1.6); 2.611(2.2); 2.590(1.5); 2.568(3.2); 2.547(4.0); 2.532(22.6); 2.526(8.2); 2.512(130.7); 2.508(260.0); 2.503(335.9); 2.499(235.6);
2.494(109.2); 2.419(1.1); 2.404(1.6); 2.395(2.0); 2.387(1.6); 2.371(1.2); 2.334(1.6); 2.330(2.1); 2.325(1.6); 2.263(0.6); 2.242(0.6); 2.050(1.3); 2.035(1.8); 2.031(2.1); 2.016(2.0);
2.012(2.0); 2.008(1.7); 1.993(1.4); 1.959(0.5); 1.827(1.0); 1.815(1.8); 1.806(2.0); 1.795(1.8); 1.784(1.1); 1.503(1.8); 1.490(2.8); 1.485(2.2); 1.477(2.3); 1.473(2.7); 1.459(1.7);
1.446(1.2); 1.435(2.1); 1.423(2.2); 1.412(2.0); 1.400(1.3); 1.377(0.3); 1.367(1.2); 1.344(1.4); 1.324(3.5); 1.312(3.2); 1.304(3.5); 1.292(2.7); 1.283(1.9); 1.271(1.4); 1.260(0.4);
1.236(0.8); 1.215(0.4); 1.170(0.4); 0.008(0.8); 0.000(24.0); −0.009(0.6)
Example 34: ¹H-NMR(400.0 MHz. DMSO): δ = 8.315(0.9); 7.432(0.3); 7.366(0.3); 7.329(6.8); 7.312(11.3); 7.308(12.2); 7.301(3.2); 7.291(16.0); 7.256(13.9); 7.235(6.5); 7.223(1.1);
7.205(8.9); 7.184(6.0); 3.902(3.1); 3.638(1.1); 3.508(0.5); 3.497(0.5); 3.328(147.8); 3.143(0.4); 3.081(0.7); 2.975(0.8); 2.944(0.3); 2.846(0.5); 2.675(1.8); 2.671(2.3); 2.666(1.7);
2.609(0.7); 2.591(1.2); 2.570(3.0); 2.549(3.3); 2.541(1.5); 2.506(307.1); 2.502(379.4); 2.497(278.2); 2.420(1.4); 2.409(1.7); 2.404(1.8); 2.394(2.0); 2.387(1.8); 2.382(1.6); 2.371(1.4);
2.333(2.1); 2.328(2.7); 2.324(2.1); 2.310(0.7); 2.293(0.4); 2.246(2.4); 2.228(0.4); 2.204(0.8); 2.131(0.6); 2.048(1.8); 2.034(2.5); 2.029(2.8); 2.025(2.2); 2.015(2.7); 2.011(2.8);
2.006(2.3); 1.992(1.9); 1.957(0.4); 1.826(1.1); 1.813(1.8); 1.805(1.9); 1.803(1.9); 1.794(1.9); 1.792(1.8); 1.781(1.3); 1.502(1.9); 1.488(3.3); 1.484(2.6); 1.476(3.0); 1.472(3.2);
1.458(2.1); 1.444(1.2); 1.433(1.9); 1.420(2.2); 1.410(1.9); 1.397(1.4); 1.379(0.4); 1.366(1.8); 1.356(0.8); 1.345(1.4); 1.334(1.7); 1.329(2.2); 1.323(3.6); 1.319(2.2); 1.311(3.3);
1.309(3.0); 1.302(3.8); 1.290(3.1); 1.282(2.4); 1.270(1.9); 1.258(0.5); 1.236(0.9); 1.200(0.4); 1.169(0.6); 1.033(0.3); 1.021(0.4); 1.013(0.4); 0.008(1.1); 0.003(5.1); 0.000(24.8); −0.009
(1.4)
Example 36: ¹H-NMR(400.0 MHz. DMSO): δ = 12.254(0.4); 12.244(0.4); 12.160(0.4); 11.984(0.3); 8.830(0.5); 8.664(0.4); 8.315(1.1); 8.217(0.5); 8.214(0.7); 8.003(0.4); 7.989(0.5);
7.938(0.4); 7.273(4.8); 7.259(5.7); 7.252(6.7); 7.238(6.1); 7.225(6.3); 7.220(2.8); 7.211(7.0); 7.203(9.6); 7.195(3.5); 7.190(8.6); 7.182(1.1); 7.123(1.2); 7.115(9.4); 7.110(2.9);
7.093(16.0); 7.089(10.1); 7.071(8.7); 7.067(13.1); 7.061(2.9); 7.050(2.2); 7.045(5.8); 7.037(0.7); 6.862(0.5); 6.856(0.6); 4.373(0.5); 4.355(0.4); 4.296(0.5); 4.278(0.5); 3.902(6.2);
3.825(1.2); 3.760(0.4); 3.325(274.2); 3.286(1.0); 3.260(0.6); 3.103(0.9); 3.086(1.3); 2.981(0.7); 2.909(3.9); 2.902(2.5); 2.848(1.1); 2.769(1.4); 2.675(1.6); 2.671(2.3); 2.667(1.7);
2.603(0.6); 2.585(1.0); 2.564(3.0); 2.542(4.2); 2.524(6.9); 2.511(155.8); 2.506(307.1); 2.502(395.0); 2.497(281.9); 2.493(134.5); 2.428(2.5); 2.417(2.7); 2.412(2.9); 2.404(3.1);
2.402(3.1); 2.394(2.9); 2.389(2.6); 2.378(2.4); 2.333(2.0); 2.329(2.5); 2.324(1.8); 2.281(0.3); 2.215(0.3); 2.026(2.2); 2.011(2.8); 2.006(2.9); 2.002(2.3); 1.992(2.7); 1.988(2.7);
1.983(2.5); 1.969(2.2); 1.800(2.6); 1.790(3.2); 1.787(3.6); 1.779(3.8); 1.777(3.7); 1.769(3.9); 1.767(3.7); 1.756(2.9); 1.490(2.3); 1.476(3.1); 1.472(2.6); 1.464(2.8); 1.460(3.2);
1.446(2.4); 1.428(2.6); 1.417(4.2); 1.404(5.0); 1.394(3.9); 1.381(2.9); 1.362(0.4); 1.351(0.8); 1.337(3.6); 1.326(3.5); 1.321(3.9); 1.316(4.5); 1.311(4.8); 1.305(3.3); 1.300(5.8);
1.290(5.4); 1.279(3.6); 1.271(2.9); 1.259(2.5); 1.236(0.9); 1.196(0.4); 1.178(0.6); 1.172(0.5); 1.159(0.4); 1.029(0.4); 1.015(0.4); 0.008(1.0); 0.000(29.8); −0.009(0.9)
Example 39: ¹H-NMR(400.0 MHz. DMSO): δ = 8.979(3.0); 8.969(3.0); 8.734(5.2); 8.721(5.2); 8.315(0.6); 7.756(11.0); 7.752(6.7); 7.744(6.8); 7.740(10.5); 7.303(3.5); 7.287(4.7);
7.283(10.3); 7.270(2.7); 7.265(6.9); 7.196(3.4); 7.193(3.2); 7.178(16.0); 7.174(11.1); 7.157(11.2); 3.902(2.9); 3.356(0.5); 3.326(191.8); 3.069(1.0); 3.058(2.2); 3.049(3.2); 3.038(3.2);
3.029(2.3); 3.018(1.1); 2.675(0.9); 2.671(1.2); 2.666(0.9); 2.662(0.4); 2.541(0.9); 2.524(3.5); 2.511(78.6); 2.506(156.9); 2.502(203.5); 2.497(143.4); 2.493(66.5); 2.338(0.4); 2.333(0.9);
2.329(1.2); 2.324(0.8); 2.137(1.6); 2.128(1.6); 2.121(1.9); 2.113(3.4); 2.105(1.9); 2.098(1.8); 2.089(1.7); 1.388(2.0); 1.375(3.2); 1.363(3.2); 1.351(2.9); 1.339(2.2); 1.282(2.4);
1.267(3.6); 1.263(3.0); 1.253(2.4); 1.248(3.5); 1.233(1.9); 0.008(0.6); 0.000(16.7); −0.009(0.5)
Example 40: ¹H-NMR(400.0 MHz. DMSO): δ = 8.779(5.2); 8.769(5.1); 8.315(0.9); 7.793(6.1); 7.773(8.0); 7.744(2.6); 7.726(6.9); 7.707(5.1); 7.666(4.7); 7.647(6.1); 7.628(2.2);
7.584(0.4); 7.551(7.4); 7.532(6.0); 7.301(5.9); 7.282(16.0); 7.263(11.8); 7.194(5.4); 7.191(3.9); 7.176(8.2); 7.171(2.7); 7.156(15.1); 7.153(15.9); 7.135(11.8); 7.078(0.4); 7.061(0.3);
3.902(2.9); 3.323(356.5); 3.293(0.6); 3.007(1.4); 2.996(3.2); 2.987(4.5); 2.977(4.4); 2.969(3.3); 2.957(1.6); 2.675(1.4); 2.671(2.0); 2.666(1.4); 2.541(1.2); 2.524(6.1); 2.510(125.0);
2.506(244.2); 2.501(314.4); 2.497(224.3); 2.493(107.5); 2.333(1.4); 2.328(1.9); 2.324(1.4); 2.053(2.4); 2.044(2.5); 2.036(3.1); 2.029(4.7); 2.021(2.9); 2.013(2.8); 2.005(2.6); 1.264(1.9);
1.250(5.6); 1.237(7.6); 1.228(6.2); 1.217(8.0); 1.201(5.4); 1.187(1.7); 0.008(0.7); 0.000(18.0); −0.008(0.6)
Example 41: ¹H-NMR(400.0 MHz. DMSO): δ = 7.937(4.9); 7.281(1.4); 7.263(3.6); 7.244(2.8); 7.229(1.3); 7.219(1.3); 7.175(1.2); 7.172(0.9); 7.157(1.9); 7.152(0.8); 7.144(3.0);
7.141(3.5); 7.123(2.5); 3.902(1.1); 3.879(16.0); 3.695(14.5); 3.324(57.1); 2.919(0.3); 2.908(0.7); 2.899(1.1); 2.888(1.1); 2.880(0.8); 2.869(0.4); 2.671(0.4); 2.524(0.9); 2.511(24.7);
2.506(49.0); 2.502(63.4); 2.497(45.2); 2.493(21.6); 2.329(0.4); 2.048(0.5); 2.040(0.6); 2.033(0.7); 2.025(1.1); 2.016(0.7); 2.009(0.6); 2.001(0.6); 1.298(0.6); 1.285(1.1); 1.274(1.0);
1.262(1.0); 1.249(0.7); 1.206(0.8); 1.191(1.3); 1.186(1.1); 1.177(1.4); 1.171(1.3); 1.157(0.7); 0.000(6.2)
Example 42: ¹H-NMR(400.0 MHz. DMSO): δ = 7.855(1.0); 7.846(1.0); 7.285(1.3); 7.267(3.3); 7.248(2.5); 7.179(1.3); 7.176(0.9); 7.161(1.9); 7.155(0.9); 7.145(2.9); 7.127(2.1);
3.902(1.1); 3.730(0.3); 3.628(7.8); 3.325(67.2); 2.924(0.6); 2.915(0.8); 2.905(0.8); 2.895(0.6); 2.671(0.4); 2.524(1.0); 2.511(25.1); 2.506(50.0); 2.502(65.0); 2.497(46.0); 2.492(21.5);
2.328(0.4); 2.225(16.0); 2.033(0.4); 2.025(0.5); 2.018(0.6); 2.009(0.9); 2.001(0.6); 1.994(0.5); 1.986(0.5); 1.297(0.5); 1.284(0.9); 1.272(0.9); 1.261(0.9); 1.248(0.6); 1.194(0.7);
1.179(1.1); 1.175(0.9); 1.165(0.7); 1.160(1.1); 1.146(0.5); 0.000(6.7)
Example 43: ¹H-NMR(400.0 MHz. DMSO): δ = 7.930(5.4); 7.284(1.5); 7.266(4.0); 7.247(3.0); 7.189(1.5); 7.179(2.8); 7.161(2.2); 7.156(1.0); 7.148(3.3); 7.145(3.8); 7.127(2.7);
4.265(1.1); 4.247(3.7); 4.230(3.8); 4.212(1.3); 3.902(0.7); 3.687(16.0); 3.326(106.0); 2.920(0.4); 2.910(0.8); 2.901(1.2); 2.890(1.2); 2.882(0.8); 2.871(0.4); 2.675(0.4); 2.671(0.5);
2.666(0.3); 2.541(0.4); 2.524(1.3); 2.511(30.1); 2.506(59.4); 2.502(76.5); 2.497(54.3); 2.493(25.6); 2.333(0.3); 2.328(0.5); 2.324(0.3); 2.039(0.6); 2.031(0.6); 2.023(0.8); 2.015(1.2);
2.007(0.7); 2.000(0.7); 1.991(0.6); 1.369(4.8); 1.352(10.0); 1.334(4.6); 1.272(0.6); 1.259(1.2); 1.247(1.1); 1.236(1.2); 1.222(1.5); 1.206(1.5); 1.202(1.2); 1.192(0.9); 1.187(1.4);
1.173(0.6); 0.000(7.3)
Example 44: ¹H-NMR(400.0 MHz. DMSO): δ = 8.645(4.1); 8.634(4.2); 8.315(0.8); 7.883(6.3); 7.881(6.6); 7.864(6.9); 7.861(6.7); 7.459(2.8); 7.456(2.9); 7.440(6.8); 7.438(6.8);
7.422(4.6); 7.419(4.5); 7.347(6.6); 7.343(7.3); 7.328(5.2); 7.324(4.8); 7.302(4.7); 7.283(12.4); 7.264(9.3); 7.194(4.5); 7.191(3.8); 7.188(4.4); 7.183(4.3); 7.176(6.9); 7.168(7.8);
7.164(16.0); 7.161(13.9); 7.149(5.3); 7.144(10.2); 7.079(0.4); 7.060(0.3); 3.902(2.8); 3.368(0.4); 3.323(289.9); 3.290(0.4); 3.008(1.2); 2.997(2.7); 2.988(3.8); 2.977(3.9); 2.968(2.9);
2.957(1.2); 2.707(0.3); 2.679(0.7); 2.675(1.4); 2.670(1.9); 2.666(1.4); 2.540(1.4); 2.524(6.2); 2.510(121.9); 2.506(241.5); 2.501(312.7); 2.497(223.1); 2.492(105.8); 2.337(0.6);
2.332(1.4); 2.328(1.8); 2.323(1.3); 2.123(1.9); 2.115(1.9); 2.107(2.4); 2.099(4.0); 2.091(2.3); 2.084(2.2); 2.075(2.1); 1.307(2.1); 1.294(3.9); 1.282(3.9); 1.270(3.6); 1.258(2.9);
1.240(3.1); 1.225(4.3); 1.221(3.8); 1.211(2.7); 1.206(4.1); 1.191(2.0); 0.008(1.1); 0.000(31.3); −0.009(0.9)
Example 45: ¹H-NMR(400.0 MHz. DMSO): δ = 8.702(4.8); 8.691(4.6); 8.314(0.7); 7.661(7.1); 7.643(7.5); 7.641(8.0); 7.454(1.7); 7.451(1.8); 7.435(5.9); 7.432(5.8); 7.418(10.4);
7.415(15.5); 7.409(12.0); 7.396(3.6); 7.390(1.9); 7.378(5.9); 7.372(4.1); 7.362(4.3); 7.359(5.4); 7.356(4.0); 7.353(4.1); 7.342(3.3); 7.336(2.8); 7.319(0.4); 7.300(5.5); 7.282(14.1);
7.263(10.6); 7.193(5.0); 7.190(4.0); 7.175(7.6); 7.170(3.7); 7.161(13.5); 7.157(16.0); 7.140(10.8); 7.077(0.5); 7.058(0.4); 6.675(0.4); 6.659(0.4); 3.902(4.6); 3.402(0.3); 3.389(0.6);
3.382(0.7); 3.327(511.7); 3.281(0.7); 3.260(0.4); 3.028(1.4); 3.017(3.3); 3.009(4.5); 2.998(4.6); 2.989(3.4); 2.978(1.5); 2.675(1.5); 2.671(1.9); 2.666(1.4); 2.541(1.5); 2.510(133.4);
2.506(246.7); 2.501(310.7); 2.497(222.0); 2.493(106.8); 2.433(0.5); 2.333(1.4); 2.328(1.8); 2.324(1.3); 2.095(2.2); 2.087(2.4); 2.080(2.9); 2.072(4.8); 2.063(2.8); 2.056(2.6); 2.048(2.4);
1.292(2.4); 1.279(4.7); 1.267(4.6); 1.255(4.4); 1.243(3.5); 1.232(3.7); 1.217(5.1); 1.213(4.6); 1.202(3.1); 1.197(4.8); 1.183(2.3); 0.008(1.6); 0.000(27.8); −0.009(1.0)
Example 46: ¹H-NMR(400.0 MHz. DMSO): δ = 8.721(4.7); 8.710(4.7); 8.315(0.7); 7.509(5.3); 7.501(1.7); 7.490(8.8); 7.486(7.3); 7.465(4.0); 7.460(4.9); 7.448(6.6); 7.443(10.0);
7.432(10.1); 7.429(11.5); 7.426(10.8); 7.407(7.8); 7.403(7.5); 7.390(5.0); 7.387(6.0); 7.384(2.8); 7.379(1.0); 7.371(2.2); 7.367(2.1); 7.299(6.0); 7.281(15.2); 7.262(11.3); 7.193(5.3);
7.190(4.0); 7.174(8.0); 7.169(3.4); 7.161(13.8); 7.157(16.0); 7.140(11.3); 7.077(0.5); 7.059(0.5); 7.047(0.3); 6.672(0.4); 6.656(0.4); 3.902(4.2); 3.323(250.1); 3.043(1.4); 3.032(3.4);
3.024(4.7); 3.012(4.7); 3.004(3.6); 2.992(1.5); 2.675(1.3); 2.670(1.8); 2.666(1.2); 2.540(1.1); 2.524(5.6); 2.510(115.9); 2.506(224.2); 2.501(286.3); 2.497(202.1); 2.492(94.5);
2.437(0.5); 2.428(0.5); 2.332(1.3); 2.328(1.8); 2.323(1.3); 2.084(2.3); 2.075(2.5); 2.068(3.0); 2.060(5.0); 2.052(2.9); 2.044(2.7); 2.036(2.5); 1.289(2.5); 1.275(4.8); 1.264(4.7);
1.252(4.6); 1.240(3.6); 1.231(3.9); 1.215(5.4); 1.211(4.7); 1.201(3.2); 1.196(5.1); 1.182(2.3); 0.008(1.2); 0.000(29.3); −0.009(0.9); −0.035(0.4)
Example 47: ¹H-NMR(400.0 MHz. DMSO): δ = 8.951(2.6); 8.941(2.5); 8.315(0.5); 7.523(6.9); 7.519(8.4); 7.502(14.7); 7.500(16.0); 7.473(0.4); 7.471(0.5); 7.456(8.2); 7.439(5.2);
7.434(4.0); 7.417(3.0); 7.381(0.3); 7.363(0.5); 7.361(0.5); 7.302(3.1); 7.298(1.6); 7.284(8.3); 7.265(6.3); 7.256(0.5); 7.202(1.4); 7.199(2.9); 7.196(2.2); 7.185(1.2); 7.180(4.4);
7.175(1.6); 7.166(7.6); 7.162(9.2); 7.145(6.1); 7.090(0.6); 7.072(0.5); 6.716(0.4); 6.712(0.5); 6.695(0.4); 3.902(3.6); 3.331(124.3); 3.326(129.4); 3.175(2.1); 3.162(2.0); 3.029(0.8);
3.018(1.3); 3.016(1.3); 3.011(1.6); 3.005(2.6); 3.001(1.6); 2.994(2.0); 2.990(1.3); 2.979(0.9); 2.675(0.8); 2.671(1.1); 2.666(0.8); 2.661(0.3); 2.541(0.8); 2.524(2.8); 2.511(66.8);
2.506(133.8); 2.502(173.7); 2.497(122.4); 2.492(57.0); 2.338(0.4); 2.333(0.8); 2.328(1.1); 2.324(0.8); 2.074(1.2); 2.065(1.3); 2.054(2.7); 2.047(1.9); 2.034(1.5); 2.026(1.4); 1.250(6.3);
1.236(3.7); 1.230(7.1); 1.214(5.4); 0.008(0.6); 0.000(20.6); −0.009(0.6)
Example 48: ¹H-NMR(400.0 MHz. DMSO): δ = 8.770(3.8); 8.757(3.8); 8.314(0.7); 7.667(5.7); 7.647(6.5); 7.461(1.4); 7.458(1.4); 7.444(7.3); 7.441(9.7); 7.425(13.7); 7.422(16.0);
7.415(10.0); 7.402(3.1); 7.396(1.6); 7.385(4.5); 7.379(3.2); 7.368(3.3); 7.365(4.1); 7.362(3.0); 7.359(3.1); 7.348(2.5); 7.342(2.0); 7.308(1.9); 7.305(1.9); 7.290(5.1); 7.286(4.9);
7.271(4.4); 7.268(3.9); 7.245(3.7); 7.241(4.5); 7.226(4.6); 7.222(5.2); 7.207(2.0); 7.203(1.9); 7.157(5.6); 7.153(5.2); 7.138(4.4); 7.134(3.9); 3.902(3.2); 3.390(0.7); 3.328(483.9);
3.294(0.9); 3.279(0.7); 3.186(1.1); 3.174(2.5); 3.166(3.1); 3.154(3.1); 3.145(2.5); 3.133(1.1); 2.675(1.2); 2.671(1.6); 2.666(1.2); 2.564(0.5); 2.541(1.1); 2.524(4.8); 2.511(103.5);
2.506(201.8); 2.502(259.0); 2.497(184.7); 2.493(88.2); 2.370(1.8); 2.360(1.9); 2.354(2.3); 2.345(3.7); 2.337(2.7); 2.329(3.4); 2.321(2.4); 1.374(2.0); 1.360(3.6); 1.349(3.7); 1.337(3.5);
1.324(2.3); 1.263(2.5); 1.248(3.9); 1.244(3.3); 1.233(2.8); 1.228(3.7); 1.214(1.9); 0.008(0.8); 0.000(20.2); −0.009(0.6)
Example 49: ¹H-NMR(400.0 MHz. DMSO): δ = 7.900(1.0); 7.889(1.0); 7.432(1.6); 7.428(1.5); 7.412(2.0); 7.409(1.9); 7.295(0.5); 7.292(0.6); 7.277(1.6); 7.273(1.5); 7.258(1.3);
7.255(1.2); 7.231(1.2); 7.227(1.4); 7.212(1.5); 7.208(1.6); 7.193(0.7); 7.189(0.6); 7.145(1.4); 7.141(1.3); 7.125(1.1); 7.122(1.0); 3.902(1.2); 3.734(0.4); 3.632(8.1); 3.324(88.1);
3.120(0.7); 3.111(0.8); 3.100(0.8); 3.091(0.7); 2.675(0.4); 2.671(0.5); 2.666(0.4); 2.524(1.4); 2.511(30.9); 2.506(61.6); 2.502(80.2); 2.497(56.8); 2.493(26.7); 2.333(0.4); 2.328(0.5);
2.324(0.4); 2.302(0.6); 2.292(0.6); 2.286(0.7); 2.277(1.2); 2.269(0.7); 2.262(0.7); 2.253(0.6); 2.230(16.0); 1.946(0.4); 1.394(0.6); 1.380(1.1); 1.369(1.1); 1.357(1.0); 1.344(0.7);
1.198(0.8); 1.183(1.1); 1.178(0.9); 1.168(0.8); 1.163(1.1); 1.148(0.6); 0.000(8.4)
Example 50: ¹H-NMR(400.0 MHz. DMSO): δ = 9.019(2.8); 9.007(2.7); 8.316(0.7); 7.558(0.4); 7.538(0.8); 7.529(6.8); 7.525(8.0); 7.507(15.7); 7.506(16.0); 7.487(0.6); 7.476(0.7);
7.463(8.1); 7.446(8.0); 7.440(4.4); 7.428(5.3); 7.424(5.9); 7.400(0.4); 7.371(0.5); 7.357(0.8); 7.344(0.5); 7.326(0.4); 7.309(1.4); 7.306(1.7); 7.291(3.9); 7.288(3.6); 7.272(3.3);
7.269(2.8); 7.250(2.8); 7.245(3.4); 7.231(3.5); 7.226(3.7); 7.212(1.7); 7.208(1.5); 7.201(0.6); 7.193(0.4); 7.170(0.3); 7.154(4.2); 7.149(3.9); 7.135(3.2); 7.130(2.9); 3.902(4.8);
3.396(0.3); 3.324(216.5); 3.263(0.4); 3.168(0.8); 3.156(1.8); 3.147(2.3); 3.140(1.7); 3.136(2.3); 3.127(2.1); 3.115(1.2); 3.098(0.9); 3.080(0.9); 3.061(0.4); 3.025(2.0); 2.779(2.0);
2.675(1.5); 2.671(1.9); 2.666(1.4); 2.546(1.7); 2.541(1.7); 2.524(5.9); 2.511(130.7); 2.506(251.2); 2.502(318.0); 2.497(222.0); 2.493(102.3); 2.348(1.4); 2.338(1.8); 2.332(2.9);
2.328(2.8); 2.324(4.0); 2.316(1.8); 2.308(1.6); 2.299(1.5); 2.281(0.4); 1.346(1.3); 1.332(3.0); 1.320(2.6); 1.310(4.6); 1.296(4.2); 1.274(2.8); 1.260(1.1); 1.235(0.4); 1.194(2.5);
1.176(5.1); 1.158(2.4); 0.008(1.3); 0.000(33.3); −0.009(1.0)
Example 51: ¹H-NMR(400.0 MHz. DMSO): δ = 8.845(8.9); 8.833(8.7); 8.579(0.4); 8.336(0.3); 8.315(1.8); 7.798(10.4); 7.779(13.7); 7.750(4.5); 7.733(11.7); 7.714(8.7); 7.672(8.2);
7.653(10.4); 7.633(3.9); 7.589(0.7); 7.571(1.4); 7.557(13.0); 7.538(10.4); 7.445(12.6); 7.442(12.6); 7.426(16.0); 7.422(15.5); 7.307(4.4); 7.304(4.7); 7.289(12.7); 7.285(12.1);
7.270(10.7); 7.266(9.5); 7.245(9.3); 7.241(10.9); 7.226(10.9); 7.222(11.9); 7.207(4.9); 7.203(4.3); 7.193(0.4); 7.164(0.7); 7.150(13.5); 7.145(12.4); 7.130(10.4); 7.126(9.3); 7.042(0.4);
3.902(8.8); 3.806(0.4); 3.422(0.5); 3.405(0.5); 3.387(0.8); 3.370(1.1); 3.325(757.2); 3.271(0.5); 3.259(0.4); 3.252(0.3); 3.240(0.4); 3.214(0.3); 3.201(0.3); 3.175(1.3); 3.165(2.6);
3.162(2.4); 3.153(5.8); 3.144(7.4); 3.133(7.6); 3.124(6.2); 3.112(3.0); 3.084(0.6); 3.068(0.4); 2.679(1.5); 2.675(2.9); 2.671(3.9); 2.666(2.7); 2.549(1.1); 2.541(2.1); 2.524(11.8);
2.511(267.7); 2.506(521.3); 2.502(666.4); 2.497(470.5); 2.492(220.2); 2.471(2.7); 2.441(0.7); 2.420(0.6); 2.385(0.5); 2.332(6.4); 2.329(6.4); 2.323(6.9); 2.315(5.6); 2.307(8.6);
2.298(5.2); 2.291(5.4); 2.282(4.5); 1.381(0.4); 1.362(0.8); 1.348(4.8); 1.335(8.7); 1.323(8.5); 1.311(8.0); 1.299(6.0); 1.270(6.4); 1.254(9.3); 1.251(8.3); 1.240(6.5); 1.235(9.0);
1.221(4.4); 1.211(0.5); 1.205(0.4); 1.193(1.1); 1.175(2.2); 1.158(1.0); 0.008(2.4); 0.000(66.4); −0.009(2.0)
Example 52: ¹H-NMR(400.0 MHz. DMSO): δ = 8.095(3.8); 7.686(1.4); 7.664(1.4); 7.246(0.8); 7.224(1.8); 7.218(1.0); 7.211(1.0); 7.203(1.4); 7.197(2.1); 7.192(2.4); 7.186(1.1);
7.180(1.0); 7.175(1.7); 7.160(0.8); 7.155(0.8); 7.056(3.5); 7.009(0.9); 7.003(0.9); 6.998(0.9); 6.993(0.9); 6.988(0.8); 6.977(0.7); 6.920(1.6); 4.550(0.7); 4.533(0.9); 4.515(0.7);
3.902(1.7); 3.867(16.0); 3.323(125.7); 3.302(0.5); 3.283(1.0); 3.261(1.0); 3.242(0.5); 2.675(0.6); 2.671(0.8); 2.666(0.5); 2.541(0.6); 2.524(2.5); 2.511(48.3); 2.506(93.1); 2.502(118.3);
2.497(82.7); 2.493(38.3); 2.333(0.5); 2.329(0.7); 2.324(0.5); 2.103(0.4); 2.080(0.5); 2.071(0.7); 2.062(0.8); 2.053(0.8); 2.043(0.6); 2.030(0.5); 2.000(0.7); 1.991(0.7); 1.981(1.4);
1.962(1.6); 1.942(1.3); 1.929(1.0); 1.920(1.4); 1.915(1.4); 1.905(0.7); 1.900(0.6); 1.887(0.4); 1.702(0.4); 1.688(0.4); 1.682(0.6); 1.669(0.9); 1.662(0.8); 1.645(1.2); 1.633(1.2);
1.613(0.6); 1.607(0.5); 1.235(0.4); 0.000(6.6)
Example 53: ¹H-NMR(400.0 MHz. DMSO): δ = 8.315(0.5); 7.971(4.4); 7.942(4.1); 7.626(0.5); 7.615(0.4); 7.597(0.5); 7.566(0.4); 7.549(0.3); 7.500(1.2); 7.478(1.2); 7.436(1.2);
7.414(1.2); 7.255(0.7); 7.246(0.7); 7.233(1.6); 7.225(1.9); 7.219(1.7); 7.213(2.0); 7.206(2.3); 7.198(2.4); 7.188(1.8); 7.184(2.2); 7.177(1.6); 7.167(0.9); 7.163(1.3); 7.011(1.5);
7.006(1.6); 7.001(1.6); 6.995(1.5); 6.990(1.3); 6.984(1.3); 5.977(1.1); 5.961(1.3); 5.946(1.4); 5.930(1.1); 5.875(0.3); 5.859(1.1); 5.843(1.3); 5.827(1.3); 5.811(1.1); 5.795(0.3);
4.555(0.8); 4.541(1.4); 4.523(1.4); 4.504(0.7); 4.083(0.4); 3.902(2.0); 3.804(16.0); 3.731(0.5); 3.325(312.6); 3.284(1.6); 3.264(1.9); 3.243(1.2); 3.223(0.5); 2.675(0.8); 2.671(1.1);
2.667(0.8); 2.541(0.7); 2.524(3.8); 2.511(75.9); 2.506(146.4); 2.502(187.9); 2.497(135.1); 2.493(66.4); 2.333(0.9); 2.329(1.1); 2.324(0.9); 2.080(0.7); 2.073(0.7); 2.063(1.0); 2.054(1.1);
2.048(1.1); 2.038(1.1); 2.030(1.2); 2.007(1.1); 1.997(0.8); 1.986(1.6); 1.976(1.3); 1.967(1.5); 1.952(1.9); 1.932(2.0); 1.912(2.5); 1.894(1.4); 1.888(1.1); 1.876(0.9); 1.866(0.5);
1.856(0.3); 1.697(0.4); 1.691(0.5); 1.678(0.9); 1.671(0.9); 1.664(1.1); 1.657(1.5); 1.636(2.4); 1.624(2.1); 1.603(1.0); 1.584(0.6); 1.575(0.4); 1.549(4.1); 1.532(4.1); 1.490(8.4);
1.474(8.1); 1.432(4.3); 1.415(4.1); 1.250(0.4); 1.236(0.7); 0.008(0.3); 0.000(8.8); −0.009(0.3)
Example 54: ¹H-NMR(400.0 MHz. DMSO): δ = 7.824(5.4); 7.627(0.3); 7.349(0.6); 7.327(1.4); 7.322(0.7); 7.306(0.9); 7.300(1.4); 7.278(0.7); 7.266(0.6); 7.261(0.7); 7.246(0.7);
7.241(0.7); 7.235(0.7); 7.230(0.7); 7.215(0.6); 7.210(0.6); 7.047(0.7); 7.042(0.8); 7.036(0.7); 7.031(0.7); 7.027(0.6); 7.021(0.6); 7.015(0.6); 6.404(1.2); 6.383(1.2); 4.544(0.7);
4.530(0.8); 4.523(0.8); 4.509(0.7); 4.137(1.2); 4.119(4.1); 4.102(4.2); 4.084(1.5); 3.902(1.1); 3.635(16.0); 3.326(164.6); 3.297(1.3); 3.278(0.4); 2.676(0.4); 2.671(0.5); 2.667(0.4);
2.511(37.3); 2.507(71.2); 2.502(90.6); 2.498(64.8); 2.493(31.1); 2.333(0.4); 2.329(0.6); 2.324(0.4); 2.149(0.4); 2.126(0.8); 2.114(0.4); 2.106(0.8); 2.083(0.5); 2.078(0.4); 2.041(0.4);
2.036(0.4); 2.029(0.5); 2.025(0.5); 2.021(0.5); 2.013(0.6); 2.005(0.4); 1.997(0.5); 1.981(0.4); 1.899(0.3); 1.890(0.4); 1.877(0.8); 1.870(1.0); 1.859(1.2); 1.854(1.2); 1.846(0.8);
1.840(0.8); 1.832(0.4); 1.823(0.6); 1.739(0.5); 1.734(0.4); 1.727(0.4); 1.722(0.5); 1.716(0.5); 1.711(0.6); 1.699(0.5); 1.688(0.4); 1.657(0.3); 1.640(0.6); 1.626(0.7); 1.608(0.6);
1.594(0.4); 1.236(0.4); 1.218(4.6); 1.201(9.6); 1.183(4.5); 0.000(3.2)
Example 55: ¹H-NMR(400.0 MHz. DMSO): δ = 8.579(0.9); 8.556(0.9); 7.354(0.6); 7.333(1.2); 7.327(0.6); 7.311(0.7); 7.306(1.2); 7.284(0.7); 7.239(0.6); 7.234(0.6); 7.220(0.6);
7.214(0.7); 7.209(0.6); 7.203(0.6); 7.189(0.6); 7.184(0.6); 7.048(0.6); 7.042(0.6); 7.037(0.6); 7.032(0.7); 7.021(0.5); 4.566(0.5); 4.561(0.4); 4.555(0.5); 4.550(0.5); 4.544(0.5);
4.534(0.5); 3.902(1.4); 3.323(114.0); 3.301(1.1); 3.293(0.7); 3.283(0.6); 3.275(0.7); 3.258(0.4); 2.669(16.0); 2.524(1.4); 2.511(40.6); 2.506(81.3); 2.502(105.6); 2.497(75.4);
2.493(35.9); 2.333(0.5); 2.328(0.7); 2.324(0.5); 2.103(0.4); 2.093(0.6); 2.084(0.6); 2.075(0.6); 2.065(0.4); 1.974(0.5); 1.967(0.5); 1.954(0.9); 1.931(0.7); 1.910(0.7); 1.905(0.7);
1.895(0.6); 1.876(0.9); 1.866(0.8); 1.857(0.6); 1.848(0.4); 1.839(0.3); 1.662(0.4); 1.652(0.4); 1.645(0.6); 1.636(0.8); 1.629(0.9); 1.618(1.1); 1.611(0.8); 1.605(0.9); 1.593(0.5);
1.236(0.4); 0.000(5.7)
Example 56: ¹H-NMR(400.0 MHz. DMSO): δ = 8.315(1.3); 7.897(0.4); 7.887(0.4); 7.876(0.3); 7.845(3.9); 7.823(3.9); 7.673(15.6); 7.659(16.0); 7.646(0.7); 7.626(1.2); 7.623(1.1);
7.616(0.9); 7.597(1.0); 7.574(0.8); 7.566(0.9); 7.556(0.7); 7.548(0.8); 7.537(0.3); 7.530(0.3); 7.486(0.3); 7.482(0.3); 7.296(2.5); 7.275(5.3); 7.269(3.1); 7.262(2.7); 7.257(3.1);
7.253(3.8); 7.247(5.7); 7.243(2.9); 7.237(2.9); 7.231(2.9); 7.226(5.4); 7.211(2.4); 7.206(2.6); 7.061(2.5); 7.055(2.5); 7.050(2.5); 7.045(2.6); 7.029(2.1); 6.692(15.3); 6.679(15.1);
4.592(1.1); 4.574(2.6); 4.560(2.7); 4.556(2.9); 4.552(2.6); 4.538(2.5); 4.520(1.1); 4.083(1.2); 3.902(8.0); 3.387(0.5); 3.358(1.4); 3.324(713.7); 3.309(6.3); 3.297(2.3); 3.287(3.8);
3.267(1.9); 2.680(1.3); 2.675(2.5); 2.671(3.3); 2.666(2.4); 2.596(0.5); 2.566(0.6); 2.541(2.4); 2.524(10.0); 2.511(207.4); 2.506(413.6); 2.502(536.4); 2.497(378.1); 2.493(175.6);
2.444(0.4); 2.419(0.4); 2.338(1.0); 2.333(2.3); 2.328(3.0); 2.324(2.2); 2.319(1.1); 2.111(1.0); 2.101(1.0); 2.093(1.2); 2.088(1.2); 2.079(2.4); 2.069(2.5); 2.060(2.2); 2.050(2.1);
2.035(1.1); 2.028(1.0); 1.997(3.2); 1.976(5.4); 1.965(4.1); 1.960(4.9); 1.939(4.1); 1.925(1.6); 1.915(2.4); 1.906(1.8); 1.887(1.7); 1.870(1.2); 1.858(0.6); 1.758(1.4); 1.744(1.5);
1.737(2.4); 1.724(2.9); 1.711(1.5); 1.705(2.5); 1.690(1.8); 1.671(1.1); 1.645(1.8); 1.624(2.8); 1.603(1.9); 1.596(1.9); 1.581(0.7); 1.573(1.2); 1.552(0.4); 1.258(0.5); 1.235(1.1);
0.854(0.3); 0.008(0.9); 0.000(28.6); −0.008(0.9)
Example 57: ¹H-NMR(400.0 MHz. DMSO): δ = 8.315(1.7); 8.232(5.4); 8.211(5.5); 7.901(0.6); 7.877(0.9); 7.854(0.7); 7.705(0.4); 7.690(0.5); 7.670(0.4); 7.646(2.6); 7.643(2.8);
7.627(5.3); 7.623(4.5); 7.614(4.1); 7.597(4.8); 7.593(2.7); 7.574(3.3); 7.566(3.8); 7.556(2.8); 7.548(3.3); 7.537(1.2); 7.534(1.1); 7.530(1.4); 7.446(0.4); 7.427(0.5); 7.408(0.4);
7.298(3.4); 7.277(7.7); 7.271(4.1); 7.255(4.7); 7.250(7.4); 7.228(4.1); 7.208(3.5); 7.203(3.7); 7.188(3.6); 7.183(3.9); 7.177(3.7); 7.172(3.7); 7.157(3.3); 7.152(3.4); 7.017(3.7);
7.012(3.7); 7.006(3.8); 7.002(3.8); 6.996(3.4); 6.990(3.1); 6.986(3.0); 4.443(1.5); 4.432(2.1); 4.426(3.0); 4.420(2.6); 4.416(3.5); 4.409(3.3); 4.403(3.5); 4.393(3.2); 4.386(1.8);
4.375(1.4); 4.306(0.5); 4.281(0.6); 4.270(0.9); 4.248(8.9); 4.239(16.0); 4.230(9.3); 4.208(1.0); 4.198(0.5); 4.083(6.1); 3.902(9.8); 3.386(0.3); 3.377(0.5); 3.369(0.6); 3.358(0.5);
3.322(527.4); 3.296(1.1); 3.290(0.9); 3.288(0.9); 3.274(0.4); 3.259(0.5); 3.234(2.2); 3.215(4.1); 3.191(4.1); 3.173(2.9); 3.150(0.4); 3.132(0.4); 3.121(0.6); 3.096(10.1); 3.090(10.2);
3.085(12.2); 3.081(10.2); 3.074(9.3); 3.050(0.7); 3.039(0.4); 2.994(0.5); 2.853(0.4); 2.680(1.4); 2.675(2.9); 2.671(4.1); 2.666(3.0); 2.619(0.3); 2.583(0.4); 2.541(2.1); 2.524(11.3);
2.511(259.3); 2.506(523.3); 2.502(683.6); 2.497(488.0); 2.493(231.7); 2.466(2.1); 2.438(0.7); 2.414(0.4); 2.391(0.4); 2.351(0.4); 2.337(1.5); 2.333(3.2); 2.328(4.2); 2.324(3.1);
2.227(0.3); 2.216(0.4); 2.052(0.7); 2.033(1.9); 2.023(2.3); 2.014(3.3); 2.003(3.2); 1.993(3.2); 1.984(3.8); 1.980(3.2); 1.965(5.1); 1.954(4.5); 1.945(7.8); 1.934(6.7); 1.925(5.7);
1.922(5.6); 1.901(3.5); 1.891(2.0); 1.880(2.3); 1.870(2.5); 1.861(3.0); 1.849(2.9); 1.841(2.9); 1.831(3.4); 1.823(2.0); 1.812(1.3); 1.731(0.4); 1.718(0.5); 1.701(0.5); 1.685(0.6);
1.652(0.8); 1.633(2.5); 1.623(1.4); 1.615(3.3); 1.592(3.9); 1.584(4.2); 1.573(4.7); 1.565(5.0); 1.554(5.5); 1.544(2.7); 1.534(2.8); 1.524(2.4); 1.517(1.4); 1.505(1.0); 1.356(0.3);
1.337(0.3); 1.282(0.4); 1.259(0.9); 1.235(3.4); 1.201(0.5); 1.183(0.5); 1.180(0.4); 1.169(1.0); 1.145(0.5); 1.140(0.4); 1.127(0.6); 1.117(0.4); 1.109(0.6); 1.099(0.4); 1.073(0.4);
1.060(0.3); 1.029(0.4); 1.013(0.5); 0.874(0.4); 0.859(0.4); 0.853(0.7); 0.836(0.5); 0.008(1.2); 0.000(38.0); −0.009(1.3)
Example 58: ¹H-NMR(400.0 MHz. DMSO): δ = 8.315(1.7); 8.121(7.1); 8.099(7.1); 8.054(0.5); 8.029(0.4); 7.901(0.4); 7.879(0.7); 7.855(0.6); 7.836(0.4); 7.809(12.5); 7.807(12.4);
7.789(13.4); 7.687(0.5); 7.646(1.1); 7.627(2.2); 7.614(1.7); 7.597(2.1); 7.574(1.4); 7.566(1.5); 7.558(1.4); 7.551(1.5); 7.530(0.7); 7.445(0.4); 7.435(0.4); 7.423(0.5); 7.414(0.6);
7.368(4.4); 7.358(6.5); 7.355(6.4); 7.346(9.7); 7.339(16.0); 7.324(6.4); 7.319(15.1); 7.297(6.4); 7.290(4.5); 7.275(4.6); 7.269(4.8); 7.264(4.7); 7.258(4.5); 7.244(4.2); 7.239(4.4);
7.160(0.3); 7.140(0.5); 7.111(7.1); 7.107(9.1); 7.101(5.4); 7.092(15.0); 7.088(14.9); 7.073(9.2); 7.069(9.3); 7.043(0.4); 6.704(11.0); 6.700(10.9); 6.685(10.8); 6.681(10.0); 5.324(0.3);
5.230(0.4); 4.894(0.4); 4.629(1.8); 4.617(2.5); 4.612(4.1); 4.600(4.4); 4.595(4.5); 4.589(4.3); 4.578(3.9); 4.572(2.5); 4.560(1.8); 4.083(3.3); 3.902(9.1); 3.509(0.4); 3.454(0.4);
3.432(0.4); 3.410(0.4); 3.393(0.5); 3.385(0.7); 3.369(1.0); 3.323(867.1); 3.286(6.1); 3.265(3.1); 3.231(0.3); 3.196(0.3); 3.184(0.4); 2.675(3.8); 2.671(4.8); 2.666(3.4); 2.609(0.6);
2.579(0.3); 2.556(0.8); 2.541(3.1); 2.524(15.3); 2.510(326.4); 2.506(628.5); 2.502(797.2); 2.497(566.5); 2.493(271.5); 2.454(1.1); 2.418(0.8); 2.370(0.5); 2.333(3.6); 2.328(4.8);
2.324(3.6); 2.293(1.2); 2.271(1.1); 2.231(0.4); 2.188(0.3); 2.154(0.4); 2.120(1.7); 2.108(1.9); 2.102(2.1); 2.095(2.2); 2.088(3.8); 2.077(3.8); 2.068(3.3); 2.063(3.1); 2.059(3.1);
2.054(2.6); 2.045(2.2); 2.035(2.1); 2.006(1.1); 1.994(5.6); 1.983(4.5); 1.974(10.7); 1.965(7.4); 1.953(7.8); 1.930(4.8); 1.917(2.8); 1.908(3.1); 1.899(4.2); 1.888(3.5); 1.880(3.4);
1.868(3.8); 1.861(2.3); 1.850(2.1); 1.839(1.1); 1.820(0.4); 1.775(2.4); 1.762(2.8); 1.756(3.2); 1.742(4.7); 1.736(3.0); 1.730(2.9); 1.723(4.5); 1.711(3.3); 1.704(2.3); 1.692(2.0);
1.685(1.1); 1.677(1.0); 1.659(3.3); 1.651(1.9); 1.635(4.2); 1.618(3.1); 1.597(1.6); 1.589(2.1); 1.566(0.9); 1.524(0.4); 1.496(0.4); 1.473(0.4); 1.456(0.4); 1.440(0.4); 1.411(0.4);
1.327(0.6); 1.312(0.6); 1.291(0.9); 1.288(0.9); 1.259(1.9); 1.236(4.6); 1.197(0.7); 1.181(0.6); 1.169(0.4); 1.143(0.4); 1.099(0.3); 1.050(0.3); 0.871(0.9); 0.853(1.0); 0.835(0.7);
0.816(0.7); 0.801(0.4); 0.008(1.1); 0.000(32.3); −0.008(1.1)
Example 59: ¹H-NMR(400.0 MHz. DMSO): δ = 7.419(3.4); 7.415(3.9); 7.392(1.0); 7.261(0.6); 7.239(1.4); 7.232(1.0); 7.225(0.7); 7.217(0.9); 7.212(1.8); 7.205(0.8); 7.200(0.7);
7.194(0.8); 7.190(0.9); 7.179(0.6); 7.174(0.6); 7.013(0.7); 7.007(0.6); 7.002(0.7); 6.997(0.7); 6.991(0.6); 6.986(0.5); 6.980(0.5); 6.618(2.8); 6.613(2.8); 4.571(0.6); 4.552(0.8);
4.533(0.6); 3.902(0.9); 3.324(77.3); 3.306(0.8); 3.285(0.9); 3.265(1.0); 3.245(0.4); 2.675(0.3); 2.671(0.4); 2.666(0.3); 2.524(1.1); 2.511(28.0); 2.506(55.8); 2.502(72.5); 2.497(52.2);
2.493(25.0); 2.328(0.4); 2.324(0.3); 2.282(16.0); 2.063(0.4); 2.054(0.6); 2.045(0.6); 2.036(0.6); 2.027(0.6); 2.012(0.4); 2.006(0.3); 1.995(0.9); 1.980(1.0); 1.973(1.4); 1.958(1.3);
1.938(1.2); 1.926(0.5); 1.916(0.6); 1.909(0.5); 1.904(0.5); 1.895(0.4); 1.889(0.4); 1.714(0.4); 1.708(0.5); 1.692(0.6); 1.681(0.4); 1.674(0.5); 1.659(0.5); 1.641(0.4); 1.629(0.5);
1.622(0.4); 1.608(0.8); 1.600(0.4); 1.586(0.5); 1.580(0.5); 1.557(0.3); 0.000(3.7)
Example 60: ¹H-NMR(400.0 MHz. DMSO): δ = 9.184(4.7); 8.316(1.2); 8.296(1.1); 7.627(0.5); 7.615(0.4); 7.598(0.5); 7.566(0.4); 7.551(0.4); 7.387(0.7); 7.382(0.7); 7.368(0.7);
7.362(0.8); 7.356(0.8); 7.350(1.1); 7.337(0.8); 7.328(1.6); 7.322(0.8); 7.306(0.9); 7.301(1.4); 7.279(0.8); 7.129(0.7); 7.124(0.8); 7.119(0.8); 7.114(0.8); 7.109(0.7); 7.103(0.6);
7.098(0.6); 4.267(0.9); 4.245(1.1); 4.223(0.9); 3.903(1.0); 3.323(54.5); 3.046(0.4); 3.021(1.0); 3.000(0.9); 2.976(0.4); 2.676(0.3); 2.671(0.5); 2.667(0.3); 2.541(0.3); 2.507(60.3);
2.502(75.8); 2.498(54.4); 2.333(0.4); 2.329(0.5); 2.325(0.3); 2.281(16.0); 2.129(0.5); 2.115(1.1); 2.099(1.4); 2.081(1.3); 2.071(0.6); 2.064(0.6); 1.821(0.3); 1.801(1.2); 1.783(1.5);
1.770(1.0); 1.763(1.4); 1.754(0.9); 1.732(0.9); 1.713(0.6); 1.707(0.6); 1.687(0.4); 1.601(0.7); 1.590(0.4); 1.580(0.7); 1.569(0.7); 1.560(0.3); 1.549(0.6); 0.000(3.7)
Example 61: ¹H-NMR(400.0 MHz. DMSO): δ = 8.996(4.5); 7.901(1.0); 7.879(1.0); 7.288(0.6); 7.267(1.4); 7.261(0.7); 7.245(0.9); 7.239(1.9); 7.234(0.7); 7.218(1.3); 7.214(0.8);
7.208(0.7); 7.202(0.7); 7.188(0.6); 7.182(0.6); 7.030(0.7); 7.023(0.7); 7.019(0.7); 7.013(0.7); 7.008(0.6); 7.002(0.6); 6.997(0.5); 4.549(0.6); 4.536(0.6); 4.532(0.7); 4.514(0.6);
3.902(0.8); 3.322(68.7); 3.309(0.8); 3.290(0.8); 3.267(0.8); 3.248(0.4); 2.676(0.4); 2.671(0.6); 2.666(0.4); 2.524(1.4); 2.511(35.6); 2.506(69.5); 2.502(89.0); 2.497(62.7); 2.493(29.2);
2.333(0.4); 2.328(0.6); 2.324(0.4); 2.140(16.0); 2.120(0.4); 2.097(0.4); 2.088(0.6); 2.079(0.6); 2.069(0.7); 2.060(0.5); 1.991(0.6); 1.974(1.0); 1.954(1.3); 1.931(1.3); 1.915(1.3);
1.896(0.6); 1.683(0.5); 1.669(0.7); 1.658(0.6); 1.651(0.8); 1.642(1.1); 1.630(1.0); 1.611(0.5); 0.008(1.1); 0.000(26.1); −0.009(0.8)
Example 62: ¹H-NMR(400.0 MHz. DMSO): δ = 8.418(1.6); 8.414(1.7); 8.406(1.7); 8.402(1.6); 8.160(1.1); 8.137(1.1); 7.358(0.6); 7.336(1.4); 7.331(0.7); 7.315(0.9); 7.309(1.4);
7.288(1.3); 7.269(0.8); 7.262(1.8); 7.257(1.9); 7.243(2.3); 7.238(2.7); 7.202(1.7); 7.190(1.6); 7.183(1.0); 7.171(0.9); 7.091(0.7); 7.085(0.7); 7.080(0.8); 7.075(0.8); 7.070(0.7);
4.671(0.4); 4.665(0.6); 4.653(0.7); 4.648(0.7); 4.643(0.7); 4.631(0.6); 4.625(0.4); 3.902(1.2); 3.324(84.7); 3.301(1.1); 3.281(0.5); 2.675(0.4); 2.671(0.5); 2.666(0.4); 2.541(0.3);
2.511(33.2); 2.506(63.8); 2.502(81.8); 2.497(58.4); 2.493(28.2); 2.333(0.4); 2.328(0.5); 2.324(0.4); 2.139(16.0); 2.119(0.7); 2.109(0.7); 2.100(0.6); 2.095(0.5); 2.090(0.5); 2.078(0.3);
1.990(0.7); 1.980(0.9); 1.970(1.1); 1.961(1.2); 1.934(1.0); 1.913(0.9); 1.905(0.6); 1.894(0.9); 1.885(0.7); 1.875(0.5); 1.865(0.6); 1.855(0.4); 1.732(0.4); 1.720(0.4); 1.713(0.6);
1.700(0.8); 1.694(0.4); 1.687(0.4); 1.680(0.7); 1.668(0.6); 1.662(0.6); 1.649(0.8); 1.628(0.7); 1.610(0.5); 1.601(0.5); 0.000(4.0)
Example 63: ¹H-NMR(400.0 MHz. DMSO): δ = 8.467(1.2); 8.461(2.0); 8.457(1.9); 8.449(2.6); 8.445(2.5); 7.627(0.5); 7.615(0.4); 7.597(0.5); 7.575(0.3); 7.566(0.4); 7.557(1.8);
7.553(1.8); 7.538(1.9); 7.534(1.8); 7.419(0.7); 7.414(0.7); 7.399(0.7); 7.394(0.8); 7.388(0.8); 7.382(1.0); 7.368(0.7); 7.359(1.5); 7.353(0.8); 7.337(0.9); 7.332(1.4); 7.310(0.7);
7.252(1.4); 7.239(1.4); 7.232(1.3); 7.220(1.2); 7.161(0.7); 7.155(0.7); 7.150(0.8); 7.145(0.8); 7.140(0.7); 7.134(0.6); 7.129(0.6); 4.330(0.8); 4.307(1.1); 4.285(0.9); 3.902(1.0);
3.324(68.1); 3.026(0.4); 3.002(1.0); 2.980(0.8); 2.713(0.4); 2.676(0.4); 2.671(0.5); 2.666(0.4); 2.541(0.3); 2.524(1.4); 2.511(35.0); 2.506(67.1); 2.502(84.9); 2.498(60.7); 2.494(29.5);
2.333(0.4); 2.329(0.5); 2.324(0.4); 2.283(16.0); 2.158(0.3); 2.151(0.4); 2.138(0.8); 2.120(0.8); 2.106(0.7); 2.099(0.7); 2.086(1.0); 2.079(0.6); 2.066(0.7); 2.061(0.6); 2.039(0.4);
1.809(0.7); 1.798(0.8); 1.790(1.0); 1.776(1.4); 1.762(1.4); 1.747(1.0); 1.728(0.6); 1.722(0.7); 1.702(0.4); 1.615(0.6); 1.605(0.4); 1.595(0.7); 1.584(0.6); 1.573(0.3); 1.564(0.5);
0.000(4.0)
Example 64: ¹H-NMR(400.0 MHz. DMSO): δ = 8.346(1.6); 8.343(1.6); 8.334(1.6); 8.332(1.6); 8.130(1.0); 8.107(1.1); 7.638(1.5); 7.636(1.5); 7.620(1.6); 7.618(1.8); 7.616(1.8);
7.368(1.9); 7.357(1.9); 7.349(1.7); 7.337(1.6); 7.299(0.7); 7.293(0.8); 7.278(0.9); 7.275(1.4); 7.268(0.8); 7.262(0.8); 7.253(1.8); 7.248(1.5); 7.242(0.9); 7.232(1.0); 7.226(1.6);
7.205(0.9); 7.077(0.8); 7.071(0.8); 7.066(0.8); 7.061(0.8); 7.056(0.7); 7.050(0.6); 7.045(0.6); 4.645(0.3); 4.632(0.4); 4.628(0.7); 4.623(0.5); 4.615(0.8); 4.610(0.8); 4.605(0.8);
4.592(0.7); 4.588(0.4); 4.574(0.3); 3.903(1.1); 3.325(102.4); 3.297(1.1); 3.278(0.5); 3.175(0.6); 3.162(0.6); 2.676(0.4); 2.671(0.5); 2.666(0.4); 2.541(0.3); 2.524(1.4); 2.511(34.5);
2.507(68.8); 2.502(88.9); 2.497(62.8); 2.493(29.4); 2.333(0.4); 2.329(0.6); 2.324(0.4); 2.153(16.0); 2.138(0.5); 2.132(0.5); 2.123(0.8); 2.114(0.8); 2.105(0.7); 2.095(0.6); 2.081(0.4);
2.000(0.8); 1.990(1.1); 1.979(1.1); 1.972(1.6); 1.960(0.7); 1.946(1.3); 1.926(0.9); 1.922(0.7); 1.914(0.8); 1.905(0.9); 1.896(0.6); 1.886(0.7); 1.876(0.5); 1.782(0.5); 1.769(0.4);
1.762(0.6); 1.749(0.9); 1.736(0.5); 1.729(0.8); 1.716(0.6); 1.709(0.4); 1.697(0.4); 1.669(0.5); 1.661(0.4); 1.646(0.7); 1.627(0.6); 1.598(0.3); 0.000(4.7)
Example 65: ¹H-NMR(400.0 MHz. DMSO): δ = 9.006(4.8); 8.349(0.9); 8.327(0.9); 8.237(5.5); 7.370(0.6); 7.349(1.2); 7.343(0.7); 7.327(0.8); 7.321(1.2); 7.300(1.2); 7.294(0.6);
7.279(0.6); 7.274(0.6); 7.268(0.6); 7.263(0.6); 7.248(0.6); 7.243(0.6); 7.094(0.6); 7.089(0.6); 7.084(0.6); 7.079(0.6); 7.073(0.5); 7.068(0.5); 7.063(0.5); 4.669(0.5); 4.664(0.4);
4.658(0.6); 4.652(0.6); 4.647(0.6); 4.635(0.5); 3.902(1.3); 3.361(1.0); 3.332(235.3); 3.301(0.6); 2.676(0.3); 2.672(0.4); 2.560(0.9); 2.542(0.3); 2.525(1.2); 2.511(27.0); 2.507(53.6);
2.502(69.1); 2.498(48.9); 2.493(22.9); 2.329(0.4); 2.155(16.0); 2.134(0.6); 2.125(0.6); 2.115(0.5); 2.106(0.4); 2.004(0.6); 1.994(0.5); 1.982(0.9); 1.978(0.8); 1.964(0.5); 1.957(0.8);
1.932(0.6); 1.915(0.8); 1.910(0.9); 1.893(0.7); 1.876(0.5); 1.867(0.4); 1.729(0.4); 1.716(0.6); 1.703(0.4); 1.697(0.5); 1.685(0.5); 1.678(0.5); 1.668(0.6); 1.648(0.6); 1.627(0.4);
0.000(3.0)
Example 66: ¹H-NMR(400.0 MHz. DMSO): δ = 8.315(1.5); 8.035(6.3); 8.012(6.3); 7.646(0.7); 7.627(1.2); 7.623(1.1); 7.614(1.0); 7.610(0.7); 7.597(1.2); 7.574(0.8); 7.566(1.0);
7.557(0.7); 7.549(0.8); 7.530(0.4); 7.457(2.9); 7.452(3.1); 7.444(3.4); 7.439(7.0); 7.434(5.1); 7.431(4.3); 7.425(5.1); 7.421(5.5); 7.418(7.4); 7.413(4.2); 7.404(4.0); 7.400(3.6);
7.322(5.1); 7.301(10.8); 7.295(5.5); 7.279(6.8); 7.274(11.9); 7.265(5.3); 7.252(9.1); 7.245(5.4); 7.239(5.2); 7.234(5.1); 7.219(4.9); 7.214(5.1); 7.204(8.5); 7.186(13.5); 7.181(10.0);
7.178(8.9); 7.169(16.0); 7.160(7.2); 7.157(7.4); 7.151(11.1); 7.149(8.7); 7.124(7.5); 7.119(7.9); 7.106(9.6); 7.101(9.6); 7.087(4.9); 7.082(4.7); 7.073(5.1); 7.067(5.1); 7.062(5.2);
7.057(5.3); 7.052(4.4); 7.046(4.1); 7.041(4.0); 4.622(1.9); 4.605(4.4); 4.591(4.9); 4.588(5.1); 4.583(4.6); 4.570(4.3); 4.552(2.0); 4.083(0.6); 3.902(6.1); 3.407(0.5); 3.391(0.5);
3.383(0.6); 3.372(0.9); 3.360(1.9); 3.325(987.0); 3.308(6.8); 3.301(7.0); 3.282(3.7); 3.267(0.5); 2.675(2.7); 2.671(3.6); 2.666(2.6); 2.583(0.5); 2.557(0.7); 2.541(2.1); 2.524(10.9);
2.511(230.3); 2.506(455.6); 2.502(586.4); 2.497(414.0); 2.493(193.0); 2.468(1.0); 2.333(2.5); 2.328(3.5); 2.324(2.5); 2.118(2.0); 2.106(1.6); 2.100(2.5); 2.095(2.8); 2.086(4.8);
2.076(4.7); 2.067(4.7); 2.062(3.4); 2.057(3.9); 2.044(2.0); 2.030(1.1); 2.020(1.2); 1.997(4.5); 1.984(5.0); 1.977(7.4); 1.969(6.3); 1.963(7.3); 1.940(7.4); 1.918(5.4); 1.910(4.9);
1.900(6.0); 1.892(5.4); 1.881(3.3); 1.872(4.0); 1.862(2.6); 1.852(1.8); 1.842(1.1); 1.725(2.5); 1.711(2.7); 1.705(4.1); 1.692(6.0); 1.686(3.1); 1.679(2.8); 1.673(5.2); 1.666(2.8);
1.659(4.8); 1.645(6.8); 1.629(5.5); 1.624(3.3); 1.609(3.5); 1.602(3.3); 1.580(2.3); 1.558(0.7); 1.259(0.5); 1.237(0.7); 0.008(1.0); 0.000(29.8); −0.009(0.9)
Example 67: ¹H-NMR(400.0 MHz. DMSO): δ = 7.301(1.3); 7.279(1.3); 7.264(0.8); 7.242(1.7); 7.236(0.9); 7.221(1.8); 7.215(2.3); 7.201(0.9); 7.194(1.4); 7.190(0.9); 7.184(0.8);
7.169(0.7); 7.164(0.7); 7.006(0.8); 7.000(0.8); 6.995(0.8); 6.989(0.8); 6.984(0.7); 6.979(0.7); 6.973(0.6); 6.228(3.5); 6.227(3.4); 4.550(0.8); 4.535(0.9); 4.532(0.9); 4.514(0.8);
4.495(0.3); 3.902(1.4); 3.324(62.8); 3.322(64.0); 3.287(0.5); 3.266(1.1); 3.246(1.2); 3.226(0.5); 2.675(0.4); 2.671(0.6); 2.666(0.4); 2.524(1.6); 2.511(36.5); 2.506(71.1); 2.502(91.0);
2.497(63.9); 2.493(29.6); 2.333(0.5); 2.328(0.6); 2.324(0.4); 2.233(16.0); 2.161(12.3); 2.070(0.4); 2.052(0.4); 2.047(0.5); 2.038(0.8); 2.029(0.8); 2.020(0.7); 2.015(0.6); 2.011(0.7);
1.997(0.5); 1.985(1.0); 1.970(1.4); 1.964(1.8); 1.950(1.8); 1.927(1.6); 1.906(0.7); 1.893(0.6); 1.884(0.5); 1.879(0.6); 1.863(0.4); 1.712(0.4); 1.697(0.4); 1.691(0.7); 1.676(0.7);
1.665(0.5); 1.657(0.7); 1.643(0.6); 1.639(0.6); 1.626(0.5); 1.619(0.7); 1.599(0.9); 1.592(0.6); 1.578(0.6); 1.572(0.6); 1.549(0.4); 0.008(1.0); 0.000(26.0); −0.009(0.9)
Example 68: ¹H-NMR(400.0 MHz. DMSO): δ = 8.314(2.3); 8.271(9.9); 8.268(11.3); 8.266(11.7); 8.263(10.5); 8.259(10.8); 8.256(11.7); 8.254(11.4); 8.251(9.6); 8.237(0.8);
8.217(8.2); 8.195(8.4); 7.689(7.9); 7.684(7.9); 7.671(9.8); 7.666(16.0); 7.661(8.2); 7.647(9.9); 7.642(9.0); 7.633(1.0); 7.626(1.5); 7.624(1.4); 7.614(1.2); 7.604(0.4); 7.597(1.5);
7.575(1.1); 7.567(1.1); 7.557(0.9); 7.549(1.0); 7.537(0.4); 7.530(0.4); 7.384(9.7); 7.379(9.7); 7.372(9.7); 7.366(13.9); 7.361(8.8); 7.354(8.5); 7.349(8.1); 7.329(6.4); 7.307(13.8);
7.301(6.8); 7.286(8.6); 7.280(13.7); 7.272(6.5); 7.266(6.6); 7.258(8.1); 7.252(6.5); 7.246(6.6); 7.240(6.4); 7.235(6.3); 7.220(5.8); 7.215(5.8); 7.073(6.2); 7.067(6.3); 7.062(6.5);
7.057(6.5); 7.052(5.6); 7.046(5.2); 7.041(5.1); 4.616(2.3); 4.604(3.1); 4.599(5.2); 4.586(5.9); 4.582(6.1); 4.577(5.5); 4.569(4.4); 4.565(5.0); 4.547(2.4); 4.083(0.9); 3.902(8.9);
3.712(0.4); 3.585(0.4); 3.555(0.3); 3.523(0.4); 3.513(0.4); 3.496(0.5); 3.484(0.5); 3.481(0.4); 3.466(0.5); 3.462(0.5); 3.456(0.6); 3.447(0.5); 3.431(0.6); 3.425(0.7); 3.399(1.5);
3.393(1.6); 3.331(2190.2); 3.313(15.1); 3.307(11.5); 3.288(5.5); 3.249(0.7); 3.242(0.5); 3.228(0.7); 3.222(0.6); 3.191(0.4); 2.676(3.2); 2.671(4.5); 2.667(3.1); 2.662(1.5); 2.573(0.7);
2.562(0.8); 2.552(0.7); 2.542(2.5); 2.525(12.2); 2.511(283.2); 2.507(560.1); 2.502(721.3); 2.498(505.7); 2.493(233.0); 2.446(0.6); 2.417(0.4); 2.401(0.4); 2.391(0.4); 2.338(1.5);
2.334(3.2); 2.329(4.3); 2.324(3.1); 2.129(2.9); 2.110(3.5); 2.106(3.5); 2.097(5.5); 2.087(6.1); 2.079(5.8); 2.069(4.5); 2.060(2.4); 2.036(1.8); 2.027(1.6); 2.005(5.1); 1.996(4.8);
1.984(8.5); 1.979(7.8); 1.966(4.6); 1.958(7.3); 1.949(4.8); 1.933(7.2); 1.928(5.2); 1.912(8.3); 1.905(7.8); 1.895(7.7); 1.887(3.9); 1.877(4.5); 1.867(3.1); 1.855(2.0); 1.847(1.5);
1.722(3.0); 1.710(3.4); 1.703(5.0); 1.690(6.6); 1.684(4.3); 1.671(7.7); 1.660(12.4); 1.650(5.9); 1.646(8.0); 1.627(4.0); 1.621(3.3); 1.607(1.9); 1.600(2.5); 1.577(0.9); 1.299(0.3);
1.259(0.5); 1.249(0.4); 1.235(0.6); 1.225(0.4); 0.008(1.2); 0.000(36.3); −0.008(1.0)
Example 69: ¹H-NMR(400.0 MHz. DMSO): δ = 8.593(16.0); 8.590(15.6); 8.443(9.5); 8.431(9.5); 8.364(5.3); 8.342(5.3); 8.315(1.2); 7.644(0.4); 7.626(0.7); 7.615(0.5); 7.597(0.6);
7.574(0.4); 7.566(0.5); 7.549(0.5); 7.342(3.2); 7.320(7.0); 7.315(3.7); 7.299(4.5); 7.293(6.9); 7.271(5.3); 7.263(3.6); 7.248(3.7); 7.243(3.9); 7.238(3.7); 7.232(3.6); 7.217(3.4);
7.212(3.3); 7.114(7.4); 7.100(10.6); 7.087(7.4); 7.072(3.9); 7.067(3.9); 7.062(4.0); 7.057(4.0); 7.052(3.5); 7.046(3.3); 7.041(3.1); 4.627(1.3); 4.609(3.1); 4.597(3.5); 4.593(3.6);
4.588(3.3); 4.576(2.9); 4.558(1.4); 3.902(3.6); 3.350(2.8); 3.322(442.2); 3.290(3.0); 2.675(2.3); 2.670(3.0); 2.666(2.2); 2.541(1.6); 2.506(380.7); 2.501(480.1); 2.497(345.2); 2.332(2.4);
2.328(3.1); 2.324(2.3); 2.136(1.6); 2.113(2.4); 2.104(3.3); 2.094(3.6); 2.086(3.7); 2.076(2.7); 2.062(1.5); 2.046(0.8); 2.030(0.9); 2.022(0.9); 2.000(3.1); 1.991(2.9); 1.979(5.1);
1.973(4.2); 1.960(2.6); 1.951(4.1); 1.946(3.5); 1.926(4.3); 1.905(5.4); 1.898(5.1); 1.888(4.5); 1.872(2.7); 1.862(2.0); 1.850(1.2); 1.841(1.0); 1.716(1.7); 1.703(2.1); 1.697(2.8);
1.684(4.1); 1.671(3.7); 1.665(4.3); 1.656(7.0); 1.644(5.6); 1.624(2.5); 1.605(1.2); 1.598(1.5); 1.576(0.6); 1.235(0.3); 0.146(0.6); 0.000(121.1); −0.008(5.0); −0.150(0.6)
Example 70: ¹H-NMR(400.0 MHz. DMSO): δ = 7.594(1.0); 7.573(1.0); 7.453(2.7); 7.441(2.8); 7.305(0.6); 7.283(1.3); 7.278(0.7); 7.262(1.3); 7.256(1.4); 7.246(0.7); 7.240(0.7);
7.235(1.3); 7.229(0.7); 7.214(0.6); 7.209(0.6); 7.057(0.6); 7.051(0.6); 7.046(0.7); 7.041(0.7); 7.036(0.6); 7.030(0.5); 7.025(0.5); 6.840(2.8); 6.828(2.7); 4.581(0.6); 4.563(0.7);
4.545(0.6); 3.902(0.9); 3.323(53.0); 3.293(0.9); 3.273(0.4); 2.675(0.3); 2.671(0.4); 2.524(1.2); 2.510(27.1); 2.506(54.4); 2.501(70.7); 2.497(50.2); 2.492(23.5); 2.333(0.3); 2.328(0.5);
2.089(0.4); 2.080(0.6); 2.071(0.7); 2.061(0.6); 2.041(16.0); 1.996(0.9); 1.985(0.7); 1.975(1.3); 1.965(1.0); 1.955(1.3); 1.950(0.7); 1.934(0.8); 1.923(0.4); 1.914(0.4); 1.904(0.6);
1.895(0.5); 1.885(0.5); 1.878(0.5); 1.760(0.4); 1.746(0.4); 1.739(0.6); 1.725(0.7); 1.719(0.4); 1.713(0.4); 1.706(0.6); 1.692(0.5); 1.626(0.5); 1.604(0.8); 1.597(0.4); 1.583(0.5);
1.575(0.5); 1.553(0.4); 0.008(0.6); 0.000(17.8); −0.009(0.6)
Example 71: ¹H-NMR(400.0 MHz. DMSO): δ = 8.050(0.9); 8.028(1.0); 7.988(5.6); 7.259(0.6); 7.237(1.7); 7.231(1.2); 7.216(1.4); 7.210(1.9); 7.205(0.7); 7.199(0.7); 7.189(0.8);
7.185(0.6); 7.179(0.6); 7.017(0.6); 7.011(0.6); 7.006(0.6); 7.001(0.6); 6.996(0.5); 6.990(0.5); 6.985(0.5); 4.557(0.6); 4.541(0.7); 4.538(0.7); 4.535(0.6); 4.520(0.6); 3.902(1.3);
3.325(94.0); 3.295(0.9); 3.276(0.9); 3.256(0.3); 2.671(0.4); 2.605(16.0); 2.524(1.1); 2.511(25.4); 2.507(50.2); 2.502(64.8); 2.497(45.6); 2.493(21.2); 2.329(0.4); 2.093(0.5); 2.084(0.5);
2.074(0.5); 2.065(0.5); 2.004(0.6); 1.990(1.1); 1.982(1.0); 1.969(1.6); 1.953(1.2); 1.927(0.5); 1.909(0.4); 1.743(0.3); 1.737(0.4); 1.722(0.5); 1.716(0.4); 1.710(0.4); 1.703(0.4);
1.689(0.3); 1.645(0.4); 1.637(0.3); 1.623(0.6); 1.615(0.4); 1.602(0.5); 1.593(0.3); 0.000(3.5)
Example 72: ¹H-NMR(400.0 MHz. DMSO): δ = 8.523(0.8); 8.501(0.8); 7.358(0.5); 7.337(1.1); 7.331(0.6); 7.315(0.7); 7.310(1.1); 7.288(0.6); 7.269(0.5); 7.264(0.5); 7.249(0.5);
7.244(0.6); 7.238(0.6); 7.233(0.6); 7.218(0.5); 7.213(0.5); 7.061(0.6); 7.055(0.6); 7.050(0.6); 7.045(0.6); 7.040(0.5); 7.034(0.5); 4.628(0.5); 4.615(0.6); 4.610(0.6); 4.606(0.5);
4.594(0.5); 3.902(0.7); 3.364(0.4); 3.346(0.8); 3.339(0.8); 3.321(63.8); 2.675(0.4); 2.670(0.5); 2.666(0.4); 2.524(1.3); 2.510(32.4); 2.506(63.7); 2.502(82.3); 2.497(59.0); 2.493(28.2);
2.421(16.0); 2.333(0.4); 2.328(0.5); 2.324(0.4); 2.123(0.5); 2.114(0.5); 2.104(0.5); 2.096(0.4); 1.992(0.4); 1.983(0.4); 1.972(0.8); 1.946(0.7); 1.921(0.8); 1.903(0.9); 1.882(0.5);
1.867(0.3); 1.720(0.4); 1.708(0.6); 1.700(0.4); 1.687(0.5); 1.674(0.5); 1.656(0.4); 1.650(0.4); 1.629(0.5); 1.609(0.4); 0.008(0.8); 0.000(23.2); −0.008(0.8)
Example 73: ¹H-NMR(400.0 MHz. DMSO): δ = 10.009(2.5); 8.348(5.4); 8.326(5.5); 8.316(2.5); 8.002(0.4); 7.992(1.5); 7.974(1.6); 7.877(0.6); 7.848(8.0); 7.845(8.1); 7.829(9.7);
7.826(9.4); 7.760(1.7); 7.752(1.9); 7.747(1.6); 7.745(1.9); 7.738(4.3); 7.727(1.7); 7.717(4.6); 7.709(5.3); 7.706(5.5); 7.695(3.2); 7.690(10.0); 7.686(10.1); 7.670(6.8); 7.667(6.4);
7.644(0.8); 7.619(7.6); 7.616(9.8); 7.600(10.9); 7.597(11.0); 7.581(4.0); 7.578(4.0); 7.566(1.0); 7.556(0.7); 7.549(0.8); 7.335(0.6); 7.327(0.6); 7.321(0.6); 7.312(0.9); 7.297(3.4);
7.275(16.0); 7.272(12.9); 7.263(5.4); 7.254(14.0); 7.249(11.8); 7.243(4.9); 7.237(4.2); 7.232(4.2); 7.227(4.6); 7.217(3.4); 7.212(3.3); 7.173(0.6); 7.152(1.3); 7.147(0.7); 7.131(0.9);
7.125(1.3); 7.119(1.1); 7.114(0.8); 7.098(1.7); 7.087(1.4); 7.075(4.9); 7.070(5.6); 7.064(4.9); 7.059(4.4); 7.049(4.1); 6.926(0.5); 6.918(0.8); 6.904(1.6); 5.245(0.4); 5.238(0.7);
5.222(0.8); 5.198(0.4); 4.966(0.5); 4.951(0.6); 4.943(0.9); 4.928(1.0); 4.921(0.5); 4.906(0.5); 4.642(1.4); 4.624(3.4); 4.610(3.6); 4.606(3.9); 4.602(3.6); 4.589(3.3); 4.571(1.5);
4.083(0.6); 3.902(10.7); 3.510(0.5); 3.495(0.7); 3.481(0.9); 3.472(0.9); 3.459(0.9); 3.444(0.7); 3.431(0.4); 3.365(2.1); 3.345(5.5); 3.322(511.6); 3.271(0.5); 2.675(3.0); 2.671(4.2);
2.666(3.0); 2.662(1.4); 2.541(2.3); 2.524(10.3); 2.510(252.5); 2.506(504.4); 2.502(659.2); 2.497(473.0); 2.493(226.3); 2.417(0.6); 2.408(0.6); 2.401(0.6); 2.391(0.8); 2.379(1.2);
2.367(0.8); 2.359(1.3); 2.346(1.0); 2.333(3.7); 2.329(4.9); 2.324(3.4); 2.320(2.1); 2.300(0.5); 2.213(0.5); 2.195(0.7); 2.189(0.8); 2.172(0.7); 2.150(2.0); 2.139(2.3); 2.132(2.5);
2.126(2.6); 2.117(4.5); 2.108(4.2); 2.099(4.0); 2.089(3.8); 2.075(2.0); 2.066(1.9); 2.033(0.8); 2.012(4.6); 2.002(4.0); 1.992(9.6); 1.985(7.2); 1.971(7.1); 1.956(5.2); 1.948(5.5);
1.941(3.5); 1.931(3.7); 1.922(3.9); 1.913(2.9); 1.903(3.2); 1.895(2.1); 1.885(2.0); 1.874(1.1); 1.783(1.9); 1.770(2.0); 1.763(2.9); 1.750(4.4); 1.737(2.2); 1.730(3.5); 1.717(2.6);
1.711(2.0); 1.698(2.2); 1.672(3.1); 1.665(2.7); 1.650(4.3); 1.630(3.3); 1.624(2.9); 1.608(1.5); 1.601(1.9); 1.578(0.7); 1.259(0.5); 1.235(0.8); 1.226(0.4); 0.146(0.8); 0.008(6.0);
0.000(184.7); −0.008(6.5); −0.150(0.9)
Example 74: ¹H-NMR(400.0 MHz. DMSO): δ = 9.800(16.0); 9.702(0.3); 8.898(1.2); 8.894(1.3); 8.885(1.3); 8.882(1.3); 8.861(0.3); 8.843(8.6); 8.840(8.9); 8.831(9.1); 8.827(8.7);
8.555(0.6); 8.551(0.7); 8.543(0.7); 8.539(0.7); 8.316(1.8); 8.167(1.2); 8.163(1.2); 8.148(1.4); 8.144(1.3); 8.108(8.6); 8.104(8.7); 8.089(9.6); 8.085(9.1); 8.038(0.4); 7.995(0.5);
7.972(0.6); 7.922(0.4); 7.905(0.4); 7.894(0.3); 7.873(0.3); 7.719(1.3); 7.707(1.4); 7.700(1.2); 7.687(1.3); 7.663(9.1); 7.651(9.2); 7.644(9.1); 7.632(8.8); 7.614(1.0); 7.598(1.2);
7.574(0.9); 7.566(1.0); 7.557(0.8); 7.551(0.8); 7.548(0.8); 7.538(0.4); 7.530(0.4); 7.506(1.0); 7.494(0.7); 7.487(0.8); 7.475(0.8); 7.356(0.3); 7.335(0.7); 7.329(0.5); 7.313(0.6);
7.308(0.7); 7.292(0.7); 7.286(0.7); 7.271(0.8); 7.265(0.7); 7.244(0.6); 7.182(0.5); 7.160(1.0); 7.155(0.6); 7.139(1.1); 7.129(3.4); 7.119(3.7); 7.113(4.2); 7.108(7.6); 7.102(5.5);
7.093(4.3); 7.086(7.0); 7.081(8.3); 7.068(3.3); 7.059(4.2); 6.911(3.5); 6.905(3.7); 6.901(3.6); 6.895(3.2); 6.885(2.8); 5.282(2.2); 5.266(2.8); 5.259(4.5); 5.243(4.7); 5.236(2.7);
5.220(2.3); 5.008(0.4); 4.994(0.4); 4.986(0.7); 4.971(0.7); 4.963(0.4); 4.551(0.3); 4.083(0.6); 3.949(2.2); 3.903(8.7); 3.543(1.8); 3.526(2.3); 3.520(2.5); 3.513(2.7); 3.503(2.6);
3.496(2.8); 3.490(2.6); 3.473(2.0); 3.366(0.6); 3.322(443.3); 3.264(0.5); 3.257(0.5); 2.680(1.3); 2.676(2.7); 2.671(3.7); 2.667(2.7); 2.662(1.3); 2.614(0.9); 2.598(1.2); 2.593(1.5);
2.589(1.5); 2.578(1.9); 2.566(2.3); 2.559(2.2); 2.555(2.4); 2.542(4.1); 2.533(3.2); 2.524(10.2); 2.511(228.4); 2.507(457.0); 2.502(595.9); 2.498(428.0); 2.493(204.5); 2.467(4.2);
2.456(1.9); 2.438(1.8); 2.424(0.6); 2.415(0.7); 2.402(0.7); 2.389(0.6); 2.380(0.8); 2.369(0.8); 2.352(0.8); 2.338(1.7); 2.333(3.0); 2.329(4.0); 2.324(3.1); 2.293(0.5); 2.213(1.6);
2.195(3.0); 2.191(3.3); 2.180(2.0); 2.173(2.1); 2.167(2.5); 2.162(2.7); 2.157(3.1); 2.146(2.1); 2.133(2.6); 2.128(2.9); 2.119(2.5); 2.115(2.5); 2.111(2.5); 2.097(3.1); 2.076(1.7);
2.014(2.5); 1.997(4.4); 1.982(3.9); 1.968(3.9); 1.952(2.2); 1.916(0.6); 1.896(0.6); 1.875(0.6); 1.854(0.5); 1.847(0.4); 1.833(0.4); 1.779(0.3); 1.768(0.4); 1.747(0.5); 1.736(1.0);
1.718(1.3); 1.706(1.9); 1.687(2.5); 1.681(2.6); 1.664(2.6); 1.659(2.3); 1.639(1.6); 1.611(0.8); 1.298(0.3); 1.259(0.5); 1.236(0.7); 0.146(0.7); 0.008(5.6); 0.000(170.9); −0.008(6.4); −0.150
(0.7)
Example 75: ¹H-NMR(400.0 MHz. DMSO): δ = 7.721(0.9); 7.701(0.9); 7.447(1.3); 7.442(1.5); 7.428(1.4); 7.423(1.6); 7.413(0.8); 7.409(0.7); 7.395(1.1); 7.392(1.2); 7.374(1.0);
7.370(0.8); 7.356(0.6); 7.335(1.3); 7.329(0.8); 7.325(0.7); 7.320(0.7); 7.313(0.9); 7.307(1.5); 7.300(0.7); 7.294(0.7); 7.287(0.9); 7.274(0.6); 7.269(0.6); 7.103(0.6); 7.097(0.7);
7.092(0.6); 7.087(0.7); 7.082(0.6); 7.077(0.5); 7.071(0.5); 7.046(1.9); 7.026(1.7); 6.968(1.0); 6.949(1.9); 6.931(0.9); 4.601(0.6); 4.588(0.7); 4.569(0.6); 3.902(0.8); 3.707(16.0);
3.367(0.4); 3.347(1.0); 3.323(75.8); 3.308(0.7); 2.675(0.3); 2.671(0.4); 2.666(0.3); 2.524(1.4); 2.511(29.0); 2.506(56.2); 2.502(72.3); 2.497(52.1); 2.493(25.3); 2.328(0.4); 2.089(0.6);
2.078(0.7); 2.069(1.1); 2.062(0.8); 2.051(0.9); 2.046(0.9); 2.040(0.8); 2.027(0.4); 1.961(0.6); 1.939(0.6); 1.929(0.4); 1.917(0.6); 1.906(0.7); 1.896(0.8); 1.890(0.7); 1.879(0.4);
1.863(0.4); 1.721(0.4); 1.717(0.5); 1.710(0.5); 1.703(0.7); 1.697(0.7); 1.691(1.0); 1.684(0.9); 1.678(0.9); 1.673(1.0); 1.664(0.6); 0.000(3.4)
Example 76: ¹H-NMR(400.0 MHz. DMSO): δ = 7.821(4.3); 7.369(0.5); 7.347(1.1); 7.342(0.6); 7.326(0.7); 7.320(1.1); 7.307(0.5); 7.299(0.7); 7.287(0.5); 7.282(0.6); 7.276(0.5);
7.271(0.5); 7.257(0.5); 7.251(0.5); 7.071(0.5); 7.065(0.6); 7.060(0.5); 7.055(0.5); 7.049(0.5); 7.044(0.5); 7.039(0.4); 6.348(0.9); 6.327(0.9); 4.476(0.5); 4.457(0.7); 4.439(0.5);
3.903(0.7); 3.747(16.0); 3.639(12.9); 3.324(75.3); 3.305(1.0); 3.285(0.4); 2.671(0.3); 2.524(0.9); 2.511(21.2); 2.507(42.1); 2.502(54.5); 2.497(38.6); 2.493(18.1); 2.078(0.6); 2.067(0.5);
2.054(0.7); 2.050(0.7); 2.040(0.6); 2.033(0.7); 2.024(0.5); 2.013(0.5); 2.010(0.4); 1.929(0.5); 1.907(0.6); 1.897(0.7); 1.879(0.8); 1.870(0.6); 1.862(0.4); 1.854(0.5); 1.686(0.5);
1.666(0.6); 1.656(0.4); 1.645(0.5); 1.638(0.6); 1.634(0.6); 1.619(0.5); 1.608(0.4); 1.604(0.5); 1.588(0.4); 0.000(2.7)
Example 77: ¹H-NMR(400.0 MHz. DMSO): δ = 7.299(0.6); 7.277(1.3); 7.272(0.7); 7.250(1.9); 7.232(1.5); 7.228(1.6); 7.215(1.6); 7.201(0.7); 7.196(0.6); 7.044(0.7); 7.034(0.7);
7.028(0.7); 7.018(0.6); 4.531(0.6); 4.514(0.7); 4.496(0.5); 3.902(0.5); 3.564(10.2); 3.330(198.2); 3.299(0.6); 3.277(0.9); 3.254(0.8); 3.236(0.4); 2.676(0.4); 2.671(0.5); 2.667(0.4);
2.541(0.4); 2.507(68.3); 2.502(86.7); 2.498(62.8); 2.333(0.4); 2.329(0.5); 2.325(0.4); 2.254(0.4); 2.072(0.4); 2.068(0.4); 2.059(0.6); 2.049(0.6); 2.041(0.6); 2.032(0.5); 2.018(0.3);
1.988(0.6); 1.978(0.6); 1.958(16.0); 1.948(1.3); 1.922(0.8); 1.915(0.6); 1.901(0.9); 1.892(0.9); 1.882(0.9); 1.873(0.5); 1.862(0.5); 1.854(0.3); 1.681(0.3); 1.676(0.5); 1.663(0.7);
1.644(0.8); 1.632(1.3); 1.618(0.9); 1.599(0.4); 1.594(0.4); 0.000(3.3)
Example 78: ¹H-NMR(400.0 MHz. DMSO): δ = 7.853(3.0); 7.844(3.1); 7.373(0.5); 7.352(1.2); 7.346(0.6); 7.331(1.1); 7.325(1.5); 7.312(0.6); 7.303(0.9); 7.296(0.5); 7.281(0.5);
7.276(0.5); 7.152(0.8); 7.132(0.8); 7.098(0.6); 7.092(0.6); 7.087(0.6); 7.082(0.6); 7.076(0.5); 7.071(0.5); 7.066(0.4); 6.686(3.3); 6.677(3.2); 4.533(0.5); 4.516(0.6); 4.499(0.5);
3.902(0.7); 3.714(16.0); 3.372(0.4); 3.328(156.0); 2.671(0.4); 2.525(1.1); 2.511(24.9); 2.507(49.3); 2.502(63.9); 2.498(45.3); 2.493(21.2); 2.329(0.4); 2.086(0.6); 2.078(0.6); 2.067(1.0);
2.061(0.8); 2.052(0.6); 2.046(0.5); 2.041(0.6); 2.023(0.3); 1.948(0.4); 1.927(0.5); 1.910(0.6); 1.907(0.6); 1.893(0.7); 1.890(0.7); 1.882(0.6); 1.871(0.6); 1.865(0.4); 1.715(0.3);
1.707(0.6); 1.698(0.6); 1.687(1.0); 1.674(0.8); 1.668(0.6); 1.658(0.5); 1.644(0.4); 0.000(3.2)
Example 79: ¹H-NMR(400.0 MHz. DMSO): δ = 7.390(1.0); 7.368(1.0); 7.300(0.6); 7.279(1.3); 7.273(0.7); 7.257(0.8); 7.251(1.3); 7.236(0.7); 7.230(1.3); 7.215(0.7); 7.211(0.7);
7.204(0.7); 7.199(0.7); 7.184(0.6); 7.180(0.6); 7.026(0.7); 7.021(0.7); 7.016(0.7); 7.011(0.7); 7.006(0.6); 6.995(0.6); 4.448(0.6); 4.431(0.7); 4.413(0.6); 4.147(1.8); 4.139(2.4);
4.137(2.4); 4.129(1.8); 4.125(1.4); 3.902(0.8); 3.323(107.8); 3.258(0.4); 3.239(0.8); 3.215(0.8); 3.196(0.4); 2.904(2.2); 2.898(1.9); 2.893(2.5); 2.889(1.9); 2.882(2.1); 2.675(0.4);
2.671(0.5); 2.666(0.4); 2.510(33.1); 2.506(63.2); 2.502(80.8); 2.497(58.0); 2.493(28.1); 2.333(0.4); 2.328(0.5); 2.324(0.4); 2.035(0.4); 2.022(0.6); 2.013(0.7); 2.004(0.7); 1.995(0.6);
1.979(0.3); 1.963(0.7); 1.954(0.7); 1.943(1.0); 1.934(1.4); 1.924(0.7); 1.912(0.9); 1.890(0.6); 1.882(0.4); 1.874(0.5); 1.866(0.8); 1.856(0.6); 1.847(0.5); 1.838(0.6); 1.828(0.3);
1.656(0.3); 1.642(0.3); 1.636(0.6); 1.623(0.7); 1.616(0.5); 1.604(0.9); 1.591(1.2); 1.566(16.0); 1.549(0.6); 1.528(0.4); 0.000(3.6)
Example 80: ¹H-NMR(400.0 MHz. DMSO): δ = 8.314(0.8); 7.734(15.1); 7.721(15.5); 7.708(0.3); 7.695(0.4); 7.664(3.6); 7.643(3.8); 7.627(1.0); 7.615(0.6); 7.597(0.6); 7.574(0.4);
7.567(0.5); 7.559(0.3); 7.551(0.4); 7.321(2.5); 7.300(7.4); 7.294(4.4); 7.277(5.5); 7.272(7.3); 7.265(3.0); 7.250(5.2); 7.245(2.6); 7.085(2.7); 7.079(3.0); 7.075(2.9); 7.070(2.9);
7.059(2.4); 7.053(2.5); 7.044(16.0); 7.031(15.1); 4.589(1.0); 4.572(2.6); 4.558(3.0); 4.554(3.0); 4.537(2.6); 4.519(1.1); 4.083(0.4); 3.902(3.7); 3.427(0.3); 3.396(0.7); 3.380(1.0);
3.357(4.3); 3.332(976.8); 3.301(3.8); 3.278(0.8); 3.265(0.4); 3.256(0.5); 3.247(0.4); 3.230(0.4); 2.676(1.4); 2.671(1.9); 2.667(1.3); 2.542(0.9); 2.525(5.0); 2.511(123.7); 2.507(243.0);
2.502(312.6); 2.498(222.3); 2.493(105.7); 2.338(0.7); 2.334(1.5); 2.329(1.9); 2.325(1.4); 2.134(1.0); 2.123(1.1); 2.117(1.3); 2.111(1.4); 2.102(2.7); 2.092(2.5); 2.084(2.5); 2.078(1.9);
2.074(2.1); 2.060(1.4); 2.051(1.4); 2.041(0.7); 2.019(2.3); 2.010(2.0); 1.999(3.8); 1.992(3.4); 1.980(4.6); 1.960(3.6); 1.958(3.7); 1.936(3.2); 1.913(3.1); 1.904(2.4); 1.893(2.1);
1.884(2.4); 1.874(1.8); 1.863(1.1); 1.854(0.7); 1.768(1.5); 1.754(1.6); 1.748(2.2); 1.735(3.3); 1.728(1.7); 1.722(1.6); 1.715(2.9); 1.702(2.2); 1.688(1.1); 1.683(1.5); 1.663(2.2);
1.657(1.2); 1.642(2.9); 1.637(1.9); 1.622(2.1); 1.614(1.9); 1.601(0.8); 1.592(1.3); 1.571(0.5); 0.000(13.9); −0.009(0.4)
Example 81: ¹H-NMR(400.0 MHz. DMSO): δ = 7.853(4.8); 7.313(1.1); 7.292(1.1); 7.237(0.6); 7.215(1.4); 7.210(1.3); 7.206(0.8); 7.194(1.1); 7.188(1.8); 7.180(0.8); 7.174(0.7);
7.167(0.8); 7.159(0.6); 7.154(0.6); 7.008(0.7); 7.002(0.7); 6.998(0.7); 6.993(0.7); 6.987(0.6); 6.982(0.6); 6.976(0.6); 4.545(0.6); 4.529(0.7); 4.511(0.5); 3.902(1.2); 3.773(0.6);
3.713(16.0); 3.676(0.8); 3.325(123.5); 3.268(0.3); 3.246(0.7); 3.227(0.8); 3.208(0.4); 2.676(0.4); 2.671(0.5); 2.666(0.4); 2.610(0.5); 2.591(0.9); 2.575(0.9); 2.557(1.3); 2.538(1.0);
2.524(1.4); 2.511(31.0); 2.506(61.6); 2.502(79.6); 2.497(56.9); 2.493(27.1); 2.476(1.0); 2.457(1.4); 2.438(0.8); 2.423(0.9); 2.403(0.5); 2.333(0.4); 2.329(0.5); 2.324(0.3); 2.069(0.4);
2.056(0.6); 2.046(0.6); 2.038(0.6); 2.029(0.5); 1.976(0.6); 1.967(0.8); 1.956(0.9); 1.948(1.1); 1.926(1.1); 1.919(1.1); 1.907(1.1); 1.890(0.5); 1.881(0.5); 1.871(0.3); 1.673(0.3);
1.667(0.5); 1.654(0.6); 1.647(0.5); 1.642(0.5); 1.634(0.8); 1.622(1.0); 1.607(0.9); 1.600(0.6); 1.587(0.5); 1.386(1.2); 1.368(2.5); 1.349(2.6); 1.330(1.4); 1.312(0.4); 0.883(0.3);
0.783(4.3); 0.765(8.8); 0.746(3.8); 0.000(4.0)
Example 82: ¹H-NMR(400.0 MHz. DMSO): δ = 7.995(0.9); 7.972(0.9); 7.358(0.6); 7.354(0.6); 7.338(1.3); 7.334(1.2); 7.328(0.6); 7.320(1.2); 7.316(1.2); 7.307(1.2); 7.301(0.7);
7.285(0.8); 7.280(1.7); 7.274(0.8); 7.266(2.0); 7.258(1.2); 7.248(1.7); 7.228(0.5); 7.223(0.5); 7.094(0.9); 7.091(0.9); 7.075(2.1); 7.072(2.1); 7.063(0.7); 7.057(1.3); 7.054(1.3);
6.804(1.5); 6.801(1.5); 6.785(1.3); 6.782(1.3); 4.594(0.5); 4.580(0.6); 4.576(0.6); 4.572(0.6); 4.559(0.5); 4.554(0.4); 3.902(0.8); 3.326(144.1); 3.290(0.9); 3.269(0.4); 2.671(0.4);
2.524(1.1); 2.511(26.0); 2.506(52.2); 2.502(68.0); 2.497(48.3); 2.492(22.7); 2.322(16.0); 2.070(0.6); 2.061(0.5); 2.052(0.5); 2.047(0.4); 2.042(0.4); 1.997(0.8); 1.980(0.9); 1.975(1.4);
1.964(1.0); 1.956(1.0); 1.946(0.4); 1.936(0.6); 1.905(0.4); 1.896(0.5); 1.885(0.4); 1.867(0.4); 1.713(0.5); 1.700(0.7); 1.694(0.4); 1.681(0.6); 1.667(0.4); 1.662(0.4); 1.649(0.4);
1.640(0.5); 1.619(0.7); 1.613(0.4); 1.599(0.4); 1.592(0.4); 0.000(3.0)
Example 83: ¹H-NMR(400.0 MHz. DMSO): δ = 8.315(0.4); 7.291(2.1); 7.270(3.3); 7.264(2.9); 7.256(1.4); 7.248(1.6); 7.242(3.0); 7.236(1.3); 7.230(1.2); 7.224(1.2); 7.221(1.4);
7.210(1.0); 7.205(0.9); 7.052(1.0); 7.046(1.1); 7.041(1.1); 7.036(1.1); 7.030(0.9); 7.026(0.9); 7.020(0.8); 4.557(0.4); 4.539(0.9); 4.527(1.0); 4.524(1.0); 4.518(0.9); 4.506(0.8);
4.489(0.4); 3.902(0.9); 3.673(0.6); 3.647(0.5); 3.643(0.5); 3.578(16.0); 3.576(15.7); 3.369(0.4); 3.365(0.4); 3.326(234.3); 3.291(0.8); 3.272(1.3); 3.248(1.3); 3.230(0.6); 2.676(0.6);
2.671(0.9); 2.666(0.7); 2.549(0.3); 2.541(0.6); 2.524(2.3); 2.511(53.8); 2.506(106.8); 2.502(138.0); 2.497(97.7); 2.493(45.8); 2.467(0.5); 2.448(0.9); 2.429(1.6); 2.411(2.6); 2.392(3.1);
2.374(3.0); 2.356(2.7); 2.337(1.9); 2.329(0.9); 2.324(0.7); 2.319(1.1); 2.100(0.5); 2.078(0.7); 2.068(0.9); 2.058(1.0); 2.050(1.0); 2.040(0.8); 2.026(0.5); 1.983(0.9); 1.973(1.3);
1.963(1.4); 1.954(1.9); 1.928(1.4); 1.907(1.0); 1.894(0.8); 1.885(1.2); 1.875(1.1); 1.866(0.7); 1.856(0.8); 1.847(0.6); 1.837(0.4); 1.688(0.5); 1.675(0.6); 1.668(0.9); 1.656(1.1);
1.649(1.1); 1.637(1.4); 1.629(1.8); 1.617(1.8); 1.598(0.7); 1.591(0.6); 1.569(0.4); 1.132(0.5); 0.954(7.4); 0.935(15.9); 0.917(7.2); 0.000(7.0)
Example 84: ¹H-NMR(400.0 MHz. DMSO): δ = 7.301(0.6); 7.280(1.3); 7.274(0.7); 7.258(1.7); 7.252(2.1); 7.231(1.5); 7.221(0.7); 7.215(0.6); 7.201(0.6); 7.196(0.6); 7.045(0.6);
7.039(0.6); 7.034(0.7); 7.029(0.7); 7.018(0.5); 7.014(0.5); 4.535(0.5); 4.518(0.6); 4.501(0.5); 3.934(0.8); 3.916(2.6); 3.902(2.3); 3.898(2.7); 3.880(0.9); 3.326(111.8); 3.298(0.5);
3.279(0.7); 3.255(0.7); 3.237(0.3); 3.175(0.9); 3.162(0.9); 2.671(0.4); 2.524(1.2); 2.511(29.0); 2.507(55.9); 2.502(71.4); 2.498(51.1); 2.333(0.3); 2.329(0.4); 2.324(0.3); 2.067(0.4);
2.058(0.6); 2.048(0.6); 2.040(0.6); 2.030(0.5); 1.999(0.3); 1.990(0.6); 1.968(16.0); 1.949(1.0); 1.925(0.7); 1.918(0.5); 1.903(0.8); 1.900(0.7); 1.895(0.8); 1.885(0.8); 1.875(0.4);
1.865(0.5); 1.855(0.3); 1.678(0.5); 1.664(0.7); 1.646(0.8); 1.634(1.2); 1.620(0.8); 1.601(0.4); 1.595(0.4); 1.274(3.8); 1.256(8.1); 1.238(3.7); 0.000(3.1)
Example 85: ¹H-NMR(400.0 MHz. DMSO): δ = 7.416(1.0); 7.394(0.9); 7.310(0.6); 7.289(1.2); 7.283(0.7); 7.273(0.7); 7.267(1.3); 7.262(1.3); 7.253(0.6); 7.248(0.7); 7.240(1.1);
7.221(0.5); 7.216(0.5); 7.071(0.6); 7.065(0.6); 7.060(0.6); 7.054(0.6); 7.049(0.5); 7.044(0.5); 7.038(0.5); 4.574(0.5); 4.562(0.6); 4.557(0.6); 4.552(0.5); 4.541(0.5); 3.902(0.8);
3.748(1.3); 3.661(15.7); 3.324(107.7); 3.298(0.9); 3.279(0.6); 3.274(0.7); 3.255(0.4); 2.675(0.3); 2.671(0.4); 2.524(1.2); 2.511(28.8); 2.506(57.4); 2.502(74.7); 2.497(53.3); 2.493(25.3);
2.333(0.3); 2.328(0.5); 2.324(0.4); 2.306(1.4); 2.097(0.4); 2.093(0.3); 2.083(0.5); 2.074(0.6); 2.065(0.5); 2.056(0.5); 2.000(0.5); 1.990(0.4); 1.981(0.9); 1.972(0.7); 1.961(1.0);
1.936(0.8); 1.922(16.0); 1.902(0.5); 1.893(0.7); 1.883(0.5); 1.873(0.4); 1.863(0.5); 1.688(0.5); 1.675(0.7); 1.667(0.4); 1.656(0.8); 1.643(1.0); 1.627(0.8); 1.607(0.4); 1.600(0.4);
0.000(3.9)
Example 86: ¹H-NMR(400.0 MHz. DMSO): δ = 8.616(0.4); 8.596(0.4); 8.313(3.9); 8.014(0.8); 8.008(0.8); 7.994(0.9); 7.988(1.1); 7.980(0.8); 7.965(0.8); 7.960(0.8); 7.867(1.0);
7.862(1.0); 7.849(1.1); 7.824(2.0); 7.820(2.1); 7.805(2.2); 7.800(2.2); 7.790(0.7); 7.767(1.9); 7.746(1.9); 7.646(0.6); 7.631(1.1); 7.626(1.1); 7.610(1.5); 7.605(1.2); 7.597(1.5);
7.584(1.5); 7.563(2.3); 7.553(4.5); 7.549(4.5); 7.534(3.5); 7.529(3.6); 7.473(1.3); 7.469(1.4); 7.455(1.8); 7.452(1.9); 7.448(1.6); 7.434(1.5); 7.430(1.4); 7.408(1.5); 7.403(1.5);
7.390(2.2); 7.387(2.5); 7.369(2.0); 7.364(1.8); 7.350(0.4); 7.334(0.4); 7.307(1.2); 7.286(2.4); 7.280(1.3); 7.264(1.6); 7.258(2.8); 7.251(1.3); 7.237(2.2); 7.230(1.4); 7.225(1.2);
7.219(1.2); 7.204(1.2); 7.200(1.2); 7.135(3.1); 7.115(2.7); 7.080(0.3); 7.059(4.0); 7.048(1.9); 7.037(5.1); 7.016(3.6); 6.997(1.4); 6.976(2.2); 6.957(3.6); 6.939(1.9); 5.669(0.5);
4.684(0.5); 4.669(1.1); 4.649(1.3); 4.633(1.1); 4.617(0.4); 4.093(3.2); 4.077(6.7); 4.061(3.9); 4.045(1.4); 4.029(0.8); 3.983(0.4); 3.956(3.1); 3.940(6.6); 3.923(3.4); 3.901(5.5);
3.370(0.9); 3.322(480.3); 3.263(1.1); 3.249(0.6); 3.233(0.5); 3.130(0.4); 3.111(0.6); 3.098(0.4); 3.079(1.8); 3.061(3.3); 3.043(1.8); 2.962(0.4); 2.746(0.3); 2.676(2.5); 2.671(3.4);
2.667(2.5); 2.575(0.4); 2.560(0.4); 2.541(1.5); 2.524(8.1); 2.511(221.8); 2.507(441.6); 2.502(572.8); 2.498(412.9); 2.493(200.0); 2.415(0.8); 2.334(2.8); 2.329(3.8); 2.324(2.8);
2.315(1.8); 2.296(3.4); 2.278(2.0); 2.200(0.3); 2.143(0.8); 2.121(1.6); 2.101(1.7); 2.084(1.4); 2.078(1.7); 2.058(0.9); 2.051(1.0); 2.035(0.8); 1.937(0.7); 1.914(1.6); 1.892(2.6);
1.879(2.1); 1.856(1.9); 1.837(2.1); 1.825(2.1); 1.819(2.7); 1.809(3.6); 1.801(2.3); 1.791(3.8); 1.774(2.7); 1.755(1.8); 1.736(1.5); 1.724(1.3); 1.714(1.4); 1.702(1.5); 1.691(1.6);
1.683(1.7); 1.663(1.6); 1.648(1.8); 1.630(2.8); 1.613(4.7); 1.594(4.6); 1.577(2.4); 1.559(0.8); 1.299(0.4); 1.259(0.5); 1.236(0.8); 1.054(0.8); 1.036(1.6); 1.017(6.2); 0.999(11.3);
0.980(5.3); 0.950(0.8); 0.932(1.4); 0.918(8.0); 0.900(16.0); 0.881(7.0); 0.855(0.4); 0.827(0.3); 0.809(0.4); 0.146(0.6); 0.008(4.8); 0.000(144.2); −0.008(5.1); −0.149(0.6)
Example 87: ¹H-NMR(400.0 MHz. DMSO): δ = 8.316(0.4); 7.316(1.6); 7.294(1.6); 7.284(1.3); 7.263(3.1); 7.257(2.1); 7.241(2.2); 7.236(3.0); 7.226(1.1); 7.214(1.6); 7.206(1.0);
7.052(1.1); 7.047(1.1); 7.042(1.2); 7.037(1.1); 7.031(1.0); 7.025(0.9); 7.020(0.9); 4.559(0.4); 4.541(0.9); 4.529(1.0); 4.524(1.0); 4.519(0.9); 4.508(0.8); 4.490(0.4); 3.902(1.8);
3.645(0.6); 3.642(0.6); 3.577(16.0); 3.575(15.3); 3.322(111.8); 3.282(0.7); 3.264(1.3); 3.240(1.3); 3.221(0.6); 2.680(0.4); 2.675(0.9); 2.671(1.3); 2.666(1.0); 2.541(0.7); 2.524(3.6);
2.511(74.9); 2.506(146.5); 2.502(187.9); 2.497(134.5); 2.493(64.4); 2.426(0.7); 2.407(1.4); 2.390(1.5); 2.371(2.5); 2.352(1.3); 2.337(0.5); 2.333(1.0); 2.328(1.3); 2.323(1.8); 2.303(2.5);
2.284(1.4); 2.267(1.2); 2.248(0.7); 2.106(0.5); 2.084(0.7); 2.074(0.9); 2.064(1.0); 2.055(1.0); 2.046(0.8); 2.033(0.5); 1.986(0.3); 1.976(0.9); 1.967(1.2); 1.956(1.5); 1.947(2.2);
1.923(1.5); 1.903(1.0); 1.897(0.7); 1.891(0.8); 1.882(1.3); 1.873(1.2); 1.863(0.7); 1.854(0.8); 1.844(0.6); 1.835(0.4); 1.682(0.5); 1.669(0.6); 1.662(0.9); 1.650(1.2); 1.643(1.2);
1.630(1.4); 1.625(1.7); 1.612(1.8); 1.594(0.9); 1.587(0.7); 1.575(0.4); 1.565(0.5); 1.376(0.5); 1.358(2.1); 1.339(4.3); 1.320(4.5); 1.302(2.4); 1.283(0.6); 0.905(0.3); 0.887(0.6);
0.868(0.4); 0.788(6.9); 0.770(14.0); 0.752(6.0); 0.008(2.2); 0.000(59.9); −0.008(2.4)
Example 88: ¹H-NMR(400.0 MHz. DMSO): δ = 8.315(1.3); 8.234(9.4); 8.211(9.5); 8.010(0.3); 7.886(0.4); 7.879(0.3); 7.798(0.3); 7.733(0.4); 7.706(11.8); 7.687(16.0); 7.633(5.5);
7.616(14.3); 7.598(12.0); 7.584(11.2); 7.566(12.9); 7.547(4.8); 7.430(0.3); 7.412(0.4); 7.382(5.6); 7.361(11.8); 7.355(6.4); 7.339(7.3); 7.334(11.4); 7.312(6.3); 7.303(6.0); 7.298(5.9);
7.283(6.0); 7.278(6.7); 7.272(6.3); 7.267(6.1); 7.252(5.6); 7.246(5.6); 7.104(6.5); 7.097(6.6); 7.093(6.8); 7.089(6.7); 7.082(6.0); 6.845(13.8); 6.827(13.0); 4.657(2.1); 4.646(3.0);
4.640(5.3); 4.629(5.9); 4.623(5.8); 4.618(5.9); 4.611(4.7); 4.607(4.9); 4.601(3.1); 4.589(2.3); 4.083(0.6); 3.902(9.1); 3.427(0.4); 3.420(0.5); 3.408(0.5); 3.404(0.5); 3.325(908.6);
3.289(8.6); 3.269(4.4); 3.242(0.4); 3.175(0.4); 3.161(0.3); 3.146(0.3); 2.675(3.6); 2.671(4.5); 2.541(2.9); 2.506(606.8); 2.502(744.2); 2.497(532.0); 2.403(0.4); 2.377(0.4); 2.333(3.5);
2.328(4.5); 2.117(2.5); 2.094(4.3); 2.085(4.7); 2.073(5.3); 2.064(6.0); 2.056(4.3); 2.044(3.7); 2.025(2.1); 2.001(7.2); 1.990(6.2); 1.980(14.2); 1.972(11.1); 1.960(9.1); 1.954(10.5);
1.935(4.6); 1.923(2.3); 1.908(2.8); 1.891(3.4); 1.881(5.1); 1.870(6.3); 1.862(4.4); 1.852(4.4); 1.840(3.3); 1.820(1.7); 1.725(0.4); 1.718(0.3); 1.697(3.1); 1.685(3.9); 1.678(4.7);
1.667(8.4); 1.659(5.5); 1.649(13.1); 1.636(11.3); 1.611(5.0); 1.590(2.4); 1.569(0.8); 1.338(0.4); 1.260(0.5); 1.250(0.5); 1.235(0.9); 0.853(0.4); 0.833(0.4); 0.813(0.4); 0.000(29.4)
Example 89: ¹H-NMR(400.0 MHz. DMSO): δ = 11.348(0.4); 8.838(7.9); 8.825(8.1); 8.446(4.1); 8.423(4.1); 8.315(0.7); 8.079(16.0); 7.902(0.3); 7.756(9.0); 7.744(8.6); 7.646(0.4);
7.627(0.7); 7.614(0.5); 7.597(0.6); 7.575(0.5); 7.566(0.5); 7.557(0.5); 7.549(0.5); 7.398(2.6); 7.376(5.4); 7.370(2.9); 7.355(3.4); 7.349(5.2); 7.328(2.8); 7.314(2.5); 7.309(2.6);
7.294(2.6); 7.289(2.9); 7.283(2.8); 7.278(2.8); 7.263(2.5); 7.258(2.5); 7.112(2.8); 7.107(2.9); 7.102(2.9); 7.096(2.9); 7.091(2.5); 7.087(2.5); 4.682(1.0); 4.672(1.3); 4.666(2.2);
4.655(2.5); 4.649(2.4); 4.643(2.6); 4.632(2.2); 4.626(1.3); 4.615(0.9); 4.083(0.4); 3.902(4.2); 3.404(0.5); 3.390(0.5); 3.361(2.5); 3.327(591.7); 3.300(2.9); 3.285(0.6); 3.271(0.4);
2.675(1.7); 2.671(2.1); 2.667(1.6); 2.579(0.4); 2.541(1.4); 2.511(146.8); 2.506(276.8); 2.502(348.7); 2.497(248.1); 2.493(121.0); 2.333(1.6); 2.329(2.1); 2.324(1.6); 2.141(1.0);
2.118(2.0); 2.108(2.1); 2.100(2.3); 2.091(2.5); 2.080(1.8); 2.068(1.3); 2.049(1.0); 2.033(1.3); 2.023(1.9); 2.014(2.3); 2.001(3.7); 1.978(4.2); 1.952(3.2); 1.931(2.1); 1.921(1.9);
1.900(2.7); 1.892(2.1); 1.881(2.4); 1.872(2.2); 1.860(1.8); 1.843(1.1); 1.832(0.8); 1.712(1.4); 1.701(1.5); 1.694(2.6); 1.683(2.8); 1.671(4.4); 1.664(4.0); 1.653(5.3); 1.643(2.7);
1.635(2.0); 1.614(1.1); 1.593(0.4); 1.236(0.5); 0.000(15.4); −0.009(0.6)
Example 90: ¹H-NMR(400.0 MHz. DMSO): δ = 8.784(11.2); 8.774(11.5); 8.335(7.7); 8.312(7.8); 8.280(0.4); 8.215(11.1); 8.212(10.8); 8.194(11.9); 7.678(8.0); 7.665(8.0); 7.658(7.8);
7.646(7.8); 7.627(1.4); 7.615(1.1); 7.597(1.2); 7.574(0.8); 7.567(0.9); 7.560(0.7); 7.557(0.8); 7.548(0.9); 7.530(0.4); 7.304(4.9); 7.299(5.2); 7.289(5.8); 7.284(5.8); 7.279(6.0);
7.273(6.6); 7.268(16.0); 7.262(6.5); 7.253(5.8); 7.247(10.6); 7.240(11.0); 7.219(5.8); 7.085(5.4); 7.079(5.5); 7.074(5.9); 7.069(5.7); 7.064(4.8); 7.059(4.4); 7.054(4.3); 4.655(2.1);
4.645(2.6); 4.638(4.7); 4.628(5.2); 4.622(5.0); 4.615(5.3); 4.605(4.2); 4.599(2.7); 4.589(2.1); 4.083(0.8); 3.902(5.5); 3.509(0.4); 3.497(0.4); 3.481(0.4); 3.448(0.6); 3.434(0.6);
3.410(0.6); 3.395(1.0); 3.382(1.1); 3.357(3.2); 3.327(1445.0); 3.299(3.8); 3.291(6.5); 3.285(5.5); 3.268(6.1); 3.249(3.5); 3.230(0.4); 3.225(0.4); 3.189(0.3); 3.176(0.5); 3.162(0.6);
2.730(0.3); 2.676(3.1); 2.671(4.1); 2.666(2.9); 2.662(1.5); 2.623(0.4); 2.588(0.6); 2.541(3.3); 2.524(12.6); 2.511(259.8); 2.507(511.6); 2.502(660.7); 2.497(473.4); 2.493(228.2);
2.456(0.7); 2.333(2.9); 2.329(3.9); 2.324(2.8); 2.133(2.1); 2.116(2.5); 2.109(3.6); 2.100(3.6); 2.097(3.4); 2.088(4.4); 2.083(3.6); 2.079(4.0); 2.071(3.9); 2.059(2.7); 2.040(1.5);
2.028(0.8); 2.007(5.2); 2.003(4.7); 1.996(5.0); 1.986(9.7); 1.977(10.2); 1.958(8.4); 1.937(4.6); 1.926(2.4); 1.907(3.9); 1.898(4.4); 1.886(4.2); 1.876(3.7); 1.867(3.8); 1.854(2.8);
1.835(1.4); 1.745(2.8); 1.735(2.9); 1.729(3.6); 1.718(4.2); 1.713(4.3); 1.709(3.8); 1.698(6.5); 1.683(9.5); 1.676(4.8); 1.667(6.4); 1.650(2.7); 1.645(3.4); 1.630(1.4); 1.622(2.0);
1.599(0.7); 1.298(0.3); 1.259(0.5); 1.236(0.6); 1.225(0.4); 0.854(0.3); 0.834(0.4); 0.008(1.0); 0.000(26.1); −0.008(0.9)
Example 91: ¹H-NMR(400.0 MHz. DMSO): δ = 8.308(1.9); 7.357(1.2); 7.335(1.2); 7.244(0.6); 7.223(1.7); 7.217(2.7); 7.211(2.5); 7.202(1.6); 7.196(1.5); 7.187(0.8); 7.182(0.9);
7.174(1.3); 7.156(0.7); 7.151(0.8); 7.138(2.5); 7.133(2.2); 7.009(0.8); 7.004(0.8); 6.998(0.8); 6.993(0.8); 6.988(0.7); 6.982(0.6); 6.977(0.6); 4.515(0.6); 4.498(0.8); 4.479(0.6);
3.898(1.4); 3.608(16.0); 3.331(170.4); 3.325(200.3); 3.291(0.6); 3.272(1.0); 3.251(1.0); 3.232(0.4); 2.677(0.6); 2.672(0.8); 2.668(0.6); 2.542(0.5); 2.526(2.4); 2.512(51.2); 2.508(102.0);
2.503(132.0); 2.499(93.0); 2.494(43.0); 2.334(0.6); 2.330(0.8); 2.325(0.6); 2.069(0.3); 2.046(0.5); 2.038(0.7); 2.029(0.9); 2.020(0.9); 2.010(0.8); 1.999(0.9); 1.988(0.9); 1.980(1.1);
1.966(1.3); 1.944(1.2); 1.923(0.8); 1.911(0.9); 1.902(1.0); 1.892(0.5); 1.883(0.5); 1.873(0.4); 1.690(0.3); 1.675(0.4); 1.670(0.5); 1.655(0.9); 1.635(1.3); 1.622(1.1); 1.616(1.1);
1.603(0.5); 1.596(0.5); 0.008(1.2); 0.000(35.8); −0.009(1.1)
Example 92: ¹H-NMR(400.0 MHz. DMSO): δ = 8.054(3.9); 7.777(1.4); 7.755(1.4); 7.258(0.8); 7.236(1.8); 7.231(1.0); 7.215(1.2); 7.209(2.1); 7.202(1.0); 7.187(1.7); 7.181(1.0);
7.176(0.9); 7.171(0.8); 7.156(0.8); 7.151(0.8); 7.012(0.9); 7.001(1.0); 6.996(0.9); 6.991(0.8); 6.985(0.7); 6.980(0.7); 4.524(0.7); 4.507(0.9); 4.489(0.7); 3.937(0.5); 3.934(0.5);
3.902(1.7); 3.882(16.0); 3.357(0.4); 3.324(147.1); 3.297(0.6); 3.277(1.1); 3.256(1.1); 3.237(0.5); 3.175(0.8); 3.162(0.8); 2.675(0.5); 2.671(0.7); 2.666(0.5); 2.541(0.6); 2.524(2.2);
2.511(47.0); 2.506(91.2); 2.502(116.9); 2.497(83.0); 2.493(39.3); 2.333(0.5); 2.328(0.7); 2.324(0.5); 2.099(0.4); 2.076(0.5); 2.067(0.8); 2.058(0.9); 2.048(0.9); 2.040(0.6); 2.026(0.4);
2.014(0.3); 1.994(0.8); 1.985(0.7); 1.974(1.4); 1.955(1.6); 1.934(1.3); 1.912(1.4); 1.907(1.4); 1.895(0.8); 1.877(0.5); 1.692(0.4); 1.671(0.7); 1.657(0.9); 1.652(1.0); 1.641(1.5);
1.630(1.3); 1.623(1.3); 1.610(0.7); 0.000(5.9)
Example 93: ¹H-NMR(400.0 MHz. DMSO): δ = 8.391(3.7); 8.369(3.8); 8.315(0.8); 7.740(7.3); 7.727(7.7); 7.644(0.5); 7.627(0.7); 7.614(0.5); 7.596(0.6); 7.575(0.5); 7.566(0.6);
7.557(0.4); 7.550(0.4); 7.335(2.5); 7.314(5.3); 7.308(2.8); 7.289(16.0); 7.276(13.6); 7.265(2.9); 7.253(2.5); 7.248(2.6); 7.233(2.5); 7.228(2.7); 7.222(2.7); 7.217(2.7); 7.203(2.4);
7.197(2.4); 7.058(2.8); 7.052(2.7); 7.047(2.8); 7.042(2.8); 7.037(2.4); 7.031(2.4); 7.026(2.2); 4.590(1.0); 4.572(2.3); 4.559(2.6); 4.555(2.6); 4.550(2.5); 4.538(2.2); 4.533(1.5);
4.519(1.0); 4.083(0.3); 3.902(2.6); 3.428(0.4); 3.403(0.4); 3.391(0.6); 3.379(0.8); 3.350(3.0); 3.327(782.2); 3.291(3.9); 3.272(1.8); 2.680(0.8); 2.675(1.7); 2.671(2.3); 2.666(1.7);
2.662(0.8); 2.559(0.5); 2.541(1.7); 2.524(6.3); 2.511(147.4); 2.506(293.5); 2.502(380.2); 2.497(271.0); 2.493(128.3); 2.333(1.7); 2.328(2.4); 2.324(1.7); 2.126(1.0); 2.107(1.5);
2.102(1.4); 2.093(2.5); 2.083(2.5); 2.074(2.5); 2.065(2.3); 2.052(1.1); 2.031(0.4); 2.015(0.3); 2.003(0.5); 1.983(2.7); 1.979(2.5); 1.973(2.2); 1.962(5.2); 1.954(5.3); 1.935(3.7);
1.914(2.5); 1.900(2.0); 1.891(3.3); 1.881(2.4); 1.871(1.7); 1.863(2.4); 1.851(1.2); 1.843(1.2); 1.833(0.6); 1.703(1.3); 1.690(1.4); 1.683(2.2); 1.670(3.0); 1.663(1.8); 1.657(1.5);
1.651(2.8); 1.632(3.4); 1.628(3.6); 1.613(3.1); 1.593(1.9); 1.586(1.8); 1.573(0.6); 1.565(1.2); 1.543(0.4); 1.234(0.5); 0.008(0.6); 0.000(17.8); −0.009(0.5)
Example 94: ¹H-NMR(400.0 MHz. DMSO): δ = 8.315(1.2); 8.171(5.9); 8.149(5.9); 7.643(0.4); 7.626(0.7); 7.614(0.6); 7.596(0.7); 7.572(10.1); 7.569(9.7); 7.553(12.3); 7.550(11.7);
7.364(4.0); 7.352(4.3); 7.349(5.2); 7.343(8.8); 7.337(5.7); 7.333(11.8); 7.330(11.5); 7.321(5.8); 7.315(16.0); 7.312(8.9); 7.297(9.2); 7.292(14.0); 7.286(4.6); 7.278(9.8); 7.273(12.2);
7.266(4.5); 7.259(7.4); 7.254(7.2); 7.240(3.6); 7.235(3.6); 7.096(3.9); 7.091(4.0); 7.086(4.2); 7.081(4.1); 7.075(3.6); 7.069(3.4); 6.816(9.9); 6.812(10.1); 6.798(9.2); 6.793(8.8);
4.644(1.5); 4.633(2.1); 4.627(3.5); 4.622(2.6); 4.615(3.8); 4.610(3.8); 4.604(3.7); 4.598(2.6); 4.593(3.3); 4.587(2.1); 4.575(1.6); 3.902(2.9); 3.457(0.3); 3.416(0.3); 3.393(0.5);
3.375(1.0); 3.356(2.7); 3.326(1134.8); 3.287(5.5); 3.268(2.8); 3.246(0.5); 2.675(2.3); 2.670(3.0); 2.666(2.2); 2.541(1.9); 2.524(8.6); 2.511(205.7); 2.506(404.5); 2.502(520.3);
2.497(369.1); 2.493(174.5); 2.427(0.6); 2.390(0.4); 2.333(2.4); 2.328(3.2); 2.324(2.3); 2.125(1.6); 2.113(1.4); 2.107(2.1); 2.101(2.2); 2.093(3.6); 2.082(3.4); 2.074(3.2); 2.064(3.0);
2.051(2.1); 2.041(1.3); 2.024(0.5); 2.014(0.8); 1.993(4.2); 1.987(3.5); 1.983(3.5); 1.972(6.8); 1.964(7.7); 1.947(5.5); 1.943(5.6); 1.922(3.7); 1.908(2.5); 1.900(3.0); 1.891(3.8);
1.880(3.5); 1.870(2.6); 1.861(3.4); 1.851(1.9); 1.841(1.6); 1.830(1.0); 1.738(2.0); 1.726(2.2); 1.719(2.8); 1.706(4.1); 1.700(2.5); 1.694(2.4); 1.687(3.9); 1.675(3.1); 1.668(3.0);
1.657(3.0); 1.652(3.6); 1.646(2.0); 1.631(4.1); 1.609(2.6); 1.592(1.2); 1.583(1.7); 1.561(0.6); 1.259(0.3); 1.236(0.5); 0.008(0.7); 0.000(20.4); −0.009(0.7)
Example 95: ¹H-NMR(400.0 MHz. DMSO): δ = 8.315(1.7); 8.149(6.8); 8.127(6.8); 7.644(0.7); 7.639(0.8); 7.626(1.1); 7.623(1.1); 7.616(1.1); 7.597(0.9); 7.574(0.7); 7.566(0.9);
7.557(0.7); 7.550(0.8); 7.525(4.8); 7.520(5.2); 7.504(8.8); 7.501(9.8); 7.485(8.2); 7.481(7.9); 7.411(0.4); 7.381(7.0); 7.379(8.4); 7.362(12.6); 7.360(16.0); 7.349(9.6); 7.344(10.4);
7.341(9.0); 7.332(9.9); 7.329(7.9); 7.310(9.2); 7.304(4.8); 7.289(6.0); 7.283(10.2); 7.274(4.8); 7.261(7.6); 7.254(4.9); 7.248(4.6); 7.243(4.5); 7.228(4.1); 7.223(4.2); 7.082(4.6);
7.076(4.6); 7.071(4.8); 7.066(4.8); 7.061(4.1); 7.055(3.8); 7.049(3.8); 7.034(11.3); 7.030(11.9); 7.015(10.3); 7.011(10.3); 4.638(1.7); 4.627(2.2); 4.621(3.9); 4.616(3.0); 4.610(4.2);
4.604(4.2); 4.598(4.3); 4.587(3.8); 4.581(2.3); 4.570(1.7); 4.083(0.5); 3.902(7.7); 3.707(0.4); 3.448(0.3); 3.434(0.5); 3.413(0.4); 3.404(0.6); 3.394(0.7); 3.385(0.7); 3.377(1.0);
3.325(989.5); 3.303(7.1); 3.281(5.6); 3.261(3.0); 2.675(2.8); 2.671(3.8); 2.666(2.7); 2.591(0.3); 2.541(2.4); 2.524(10.5); 2.511(235.4); 2.506(467.1); 2.502(606.7); 2.497(433.6);
2.493(206.4); 2.338(1.4); 2.333(2.7); 2.329(3.6); 2.324(2.6); 2.122(1.7); 2.099(3.1); 2.090(3.4); 2.079(3.8); 2.070(4.2); 2.061(3.3); 2.048(2.5); 2.030(1.3); 2.004(1.1); 1.993(5.2);
1.982(4.6); 1.973(10.0); 1.963(7.8); 1.953(6.4); 1.947(6.7); 1.928(4.0); 1.915(2.3); 1.898(3.3); 1.889(3.9); 1.879(4.3); 1.871(2.7); 1.861(3.4); 1.850(2.2); 1.830(1.1); 1.806(0.3);
1.703(2.2); 1.690(2.6); 1.684(3.8); 1.672(4.7); 1.665(5.2); 1.652(6.6); 1.648(6.9); 1.634(6.8); 1.614(2.9); 1.608(3.1); 1.596(1.0); 1.589(1.8); 1.566(0.6); 1.259(0.5); 1.234(0.7);
0.008(1.1); 0.000(30.8); −0.009(0.9)
Example 96: ¹H-NMR(400.0 MHz. DMSO): δ = 8.315(0.8); 7.782(3.1); 7.761(3.1); 7.709(13.1); 7.696(13.5); 7.626(0.5); 7.614(0.4); 7.597(0.4); 7.574(0.3); 7.566(0.4); 7.548(0.4);
7.319(2.1); 7.298(6.0); 7.292(4.2); 7.276(5.0); 7.271(6.9); 7.265(2.6); 7.260(2.5); 7.249(3.0); 7.240(2.1); 7.079(16.0); 7.066(15.2); 4.592(0.9); 4.575(2.1); 4.562(2.3); 4.558(2.4);
4.554(2.2); 4.541(2.1); 4.523(1.0); 3.902(3.9); 3.387(0.3); 3.370(0.6); 3.355(2.1); 3.325(516.5); 3.296(2.0); 2.675(1.4); 2.671(1.9); 2.666(1.4); 2.548(0.5); 2.541(1.1); 2.524(5.4);
2.511(123.3); 2.506(246.7); 2.502(321.7); 2.497(229.2); 2.493(108.6); 2.333(1.4); 2.329(2.0); 2.324(1.4); 2.134(0.9); 2.122(0.9); 2.116(1.1); 2.110(1.2); 2.101(2.2); 2.091(2.0);
2.083(2.0); 2.073(1.7); 2.059(1.1); 2.050(1.1); 2.038(0.5); 2.017(2.0); 2.003(2.2); 1.996(3.0); 1.988(3.1); 1.983(3.2); 1.979(3.0); 1.959(3.4); 1.937(2.7); 1.915(2.5); 1.906(2.0);
1.896(1.7); 1.886(1.9); 1.876(1.4); 1.865(0.9); 1.856(0.5); 1.768(1.2); 1.755(1.2); 1.748(1.8); 1.735(2.7); 1.729(1.3); 1.722(1.3); 1.716(2.3); 1.702(1.8); 1.684(1.3); 1.667(1.8);
1.660(1.1); 1.645(2.3); 1.627(1.6); 1.618(1.5); 1.605(0.6); 1.597(1.0); 1.575(0.4); 1.234(0.5); 0.008(0.5); 0.000(14.8); −0.009(0.4)
Example 97: ¹H-NMR(400.0 MHz. DMSO): δ = 8.315(0.3); 8.171(1.8); 8.149(1.8); 7.953(2.4); 7.628(0.3); 7.587(6.6); 7.313(0.9); 7.292(1.8); 7.286(1.0); 7.270(1.2); 7.264(1.8);
7.243(0.9); 7.214(0.9); 7.209(1.0); 7.194(1.0); 7.189(1.1); 7.184(1.1); 7.178(1.0); 7.163(0.9); 7.158(0.9); 7.018(1.1); 7.013(1.2); 4.516(0.4); 4.497(0.9); 4.485(1.0); 4.465(0.9);
4.446(0.3); 3.902(1.6); 3.751(0.7); 3.326(224.2); 3.292(0.7); 3.272(1.4); 3.248(1.3); 3.229(0.6); 2.891(16.0); 2.732(14.3); 2.675(0.8); 2.671(1.0); 2.667(0.7); 2.541(0.8); 2.506(130.9);
2.502(161.0); 2.497(114.4); 2.333(0.7); 2.329(1.0); 2.086(0.4); 2.064(0.7); 2.053(0.9); 2.044(1.1); 2.035(1.1); 2.025(0.8); 2.013(0.5); 1.994(0.4); 1.963(0.9); 1.953(1.2); 1.943(1.4);
1.933(1.8); 1.908(1.5); 1.887(1.0); 1.878(1.1); 1.872(1.2); 1.863(1.4); 1.854(0.8); 1.845(0.6); 1.836(0.6); 1.656(0.5); 1.636(0.8); 1.624(1.4); 1.606(2.0); 1.593(1.7); 1.575(0.8);
1.569(0.8); 1.548(0.4); 0.000(7.1); −0.008(0.3)
Example 98: ¹H-NMR(400.0 MHz. DMSO): δ = 8.315(1.1); 8.078(3.9); 8.056(4.0); 7.710(0.4); 7.626(0.5); 7.614(0.4); 7.596(0.5); 7.574(0.3); 7.566(0.5); 7.548(0.5); 7.413(0.3);
7.334(0.4); 7.308(2.2); 7.303(2.4); 7.294(2.9); 7.288(2.6); 7.283(2.8); 7.273(6.9); 7.267(3.5); 7.260(3.6); 7.257(5.1); 7.251(5.9); 7.246(6.3); 7.242(7.7); 7.238(8.8); 7.232(3.1);
7.223(9.0); 7.219(16.0); 7.200(9.8); 7.159(4.8); 7.156(3.7); 7.151(4.2); 7.148(5.1); 7.141(6.5); 7.132(6.4); 7.129(6.5); 7.123(2.4); 7.113(3.0); 7.111(3.0); 7.094(11.5); 7.090(13.7);
7.073(10.5); 7.062(7.9); 7.042(5.2); 6.827(5.6); 6.824(5.7); 6.808(5.1); 6.805(4.9); 4.697(0.8); 4.685(1.1); 4.679(1.9); 4.667(2.2); 4.662(2.1); 4.657(2.1); 4.645(1.9); 4.639(1.2);
4.627(0.9); 4.344(0.9); 4.082(0.3); 3.902(7.3); 3.810(5.2); 3.773(7.6); 3.629(7.4); 3.592(5.2); 3.321(271.2); 3.291(3.5); 3.269(1.6); 3.175(0.6); 3.162(0.7); 2.885(0.9); 2.679(0.9);
2.675(1.9); 2.670(2.6); 2.666(1.9); 2.661(0.9); 2.541(1.2); 2.524(6.4); 2.510(160.4); 2.506(323.6); 2.501(423.9); 2.497(303.5); 2.492(144.6); 2.352(1.0); 2.337(1.1); 2.333(2.1);
2.328(2.8); 2.323(2.0); 2.113(1.1); 2.091(1.7); 2.080(2.0); 2.071(2.2); 2.061(2.6); 2.052(1.7); 2.040(1.4); 2.021(0.8); 1.986(2.0); 1.974(3.8); 1.966(5.2); 1.953(5.6); 1.945(3.7);
1.931(2.9); 1.907(0.6); 1.887(0.8); 1.880(1.1); 1.870(2.0); 1.859(2.0); 1.843(1.9); 1.829(1.5); 1.813(0.6); 1.672(1.1); 1.660(1.2); 1.653(2.3); 1.641(2.6); 1.634(2.8); 1.621(3.6);
1.613(3.8); 1.602(3.6); 1.597(2.9); 1.582(1.6); 1.575(1.9); 1.562(0.6); 1.555(1.1); 1.532(0.4); 1.236(0.5); 0.146(0.5); 0.008(4.2); 0.000(125.6); −0.009(4.4); −0.150(0.5)
Example 99: ¹H-NMR(400.0 MHz. DMSO): δ = 7.845(5.4); 7.397(1.1); 7.375(1.1); 7.268(0.6); 7.247(1.3); 7.241(0.8); 7.231(0.8); 7.225(1.4); 7.220(1.4); 7.211(0.8); 7.206(0.8);
7.198(1.2); 7.180(0.6); 7.175(0.6); 7.028(0.7); 7.024(0.7); 7.019(0.7); 7.013(0.7); 7.008(0.6); 6.996(0.6); 4.533(0.6); 4.517(0.7); 4.500(0.5); 3.902(1.6); 3.858(0.5); 3.849(0.3);
3.809(16.0); 3.785(0.5); 3.596(0.4); 3.323(124.0); 3.298(0.6); 3.280(0.8); 3.258(0.8); 3.240(0.4); 2.675(0.5); 2.671(0.7); 2.666(0.5); 2.541(0.5); 2.536(0.4); 2.524(1.8); 2.510(42.8);
2.506(86.5); 2.502(113.9); 2.497(83.0); 2.493(40.8); 2.333(0.5); 2.328(0.7); 2.324(0.5); 2.063(0.4); 2.053(0.6); 2.044(0.7); 2.036(0.7); 2.027(0.6); 2.008(0.7); 1.998(0.6); 1.985(1.0);
1.963(0.9); 1.940(0.9); 1.920(1.0); 1.886(0.3); 1.880(0.4); 1.679(0.4); 1.672(0.6); 1.659(0.8); 1.652(1.0); 1.634(1.2); 1.621(0.7); 0.000(5.1)
Example 100: ¹H-NMR(400.0 MHz. DMSO): δ = 7.610(1.1); 7.588(1.1); 7.273(0.6); 7.251(1.4); 7.246(1.3); 7.241(0.8); 7.230(1.0); 7.224(1.8); 7.214(0.8); 7.209(0.8); 7.203(0.8);
7.194(0.7); 7.189(0.6); 7.019(0.7); 7.013(0.7); 7.008(0.7); 7.003(0.7); 6.998(0.6); 6.992(0.6); 6.987(0.6); 6.965(2.9); 6.961(2.9); 6.684(0.6); 6.597(3.3); 6.593(3.2); 4.539(0.6);
4.521(0.8); 4.517(0.7); 4.503(0.6); 3.902(1.8); 3.665(1.6); 3.596(16.0); 3.324(127.1); 3.303(0.4); 3.282(0.5); 3.262(0.9); 3.243(1.0); 3.222(0.4); 2.675(0.5); 2.671(0.6); 2.666(0.5);
2.541(0.4); 2.524(1.7); 2.511(40.1); 2.506(78.6); 2.502(101.0); 2.497(71.4); 2.493(33.7); 2.333(0.4); 2.328(0.6); 2.324(0.4); 2.058(0.4); 2.049(0.6); 2.040(0.6); 2.031(0.6); 2.022(0.6);
2.006(0.3); 1.987(0.5); 1.979(0.5); 1.969(1.2); 1.964(1.0); 1.959(1.0); 1.948(2.2); 1.928(1.6); 1.909(0.5); 1.900(0.6); 1.891(0.5); 1.880(0.5); 1.722(0.3); 1.707(0.4); 1.701(0.6);
1.687(0.7); 1.675(0.4); 1.667(0.6); 1.654(0.4); 1.649(0.4); 1.635(0.4); 1.613(0.4); 1.592(0.7); 1.584(0.5); 1.570(0.5); 1.562(0.5); 1.541(0.3); 0.000(5.3)
Example 101: ¹H-NMR(400.0 MHz. DMSO): δ = 8.315(1.5); 7.855(4.4); 7.834(4.4); 7.759(0.4); 7.643(0.6); 7.627(1.2); 7.615(16.0); 7.602(15.9); 7.574(0.6); 7.566(0.6); 7.556(0.5);
7.553(0.5); 7.548(0.6); 7.431(0.3); 7.408(0.4); 7.309(2.8); 7.288(6.0); 7.281(4.7); 7.274(3.8); 7.266(4.7); 7.260(8.2); 7.254(3.6); 7.249(3.6); 7.243(3.7); 7.239(3.9); 7.229(2.9);
7.224(3.2); 7.210(0.5); 7.202(0.4); 7.195(0.4); 7.135(0.4); 7.120(15.9); 7.107(15.1); 7.078(3.1); 7.071(3.2); 7.067(3.2); 7.062(3.2); 7.056(2.6); 7.051(2.5); 7.045(2.4); 6.965(0.3);
6.961(0.3); 6.597(0.4); 6.593(0.4); 4.594(1.3); 4.577(2.9); 4.563(3.2); 4.560(3.4); 4.556(3.1); 4.542(2.8); 4.524(1.3); 3.902(6.6); 3.832(0.5); 3.818(0.3); 3.805(1.0); 3.596(1.7);
3.323(549.3); 3.285(2.3); 3.268(0.5); 2.675(2.9); 2.670(4.0); 2.666(2.9); 2.541(2.0); 2.524(10.1); 2.510(238.5); 2.506(479.1); 2.501(629.7); 2.497(453.1); 2.492(216.8); 2.333(2.9);
2.328(4.0); 2.324(2.9); 2.129(1.1); 2.118(1.4); 2.111(1.4); 2.106(1.6); 2.097(2.8); 2.087(2.8); 2.078(2.6); 2.069(2.5); 2.054(1.6); 2.046(1.6); 2.033(0.8); 2.012(3.1); 2.002(3.4);
1.991(4.7); 1.983(6.5); 1.972(2.9); 1.961(4.6); 1.955(3.6); 1.940(4.1); 1.930(2.9); 1.921(3.7); 1.912(3.2); 1.903(2.7); 1.893(2.8); 1.883(2.0); 1.873(1.5); 1.862(0.9); 1.781(1.5);
1.768(1.7); 1.761(2.4); 1.748(3.6); 1.735(1.9); 1.729(3.0); 1.715(2.2); 1.696(1.4); 1.665(2.2); 1.643(3.1); 1.624(2.2); 1.616(2.1); 1.602(1.1); 1.594(1.5); 1.572(0.6); 1.258(0.5);
1.235(0.7); 0.146(0.7); 0.008(5.4); 0.000(162.7); −0.008(6.1); −0.150(0.7)
Example 102: ¹H-NMR(400.0 MHz. DMSO): δ = 9.183(0.4); 8.843(16.0); 8.842(15.8); 8.315(1.1); 8.224(3.9); 8.203(4.0); 7.644(1.3); 7.632(1.3); 7.627(2.3); 7.624(2.0); 7.615(1.8);
7.597(2.1); 7.574(1.5); 7.566(1.6); 7.556(1.3); 7.549(1.5); 7.538(0.6); 7.530(0.6); 7.392(2.5); 7.387(2.6); 7.372(2.7); 7.367(2.9); 7.361(2.9); 7.356(3.1); 7.347(3.2); 7.341(2.9);
7.336(3.2); 7.326(6.4); 7.320(3.4); 7.305(4.2); 7.299(6.1); 7.277(3.2); 7.131(2.6); 7.125(2.7); 7.120(2.9); 7.114(2.8); 7.109(2.4); 7.103(2.4); 7.098(2.3); 7.057(0.3); 4.315(0.9);
4.294(3.1); 4.271(3.9); 4.250(3.3); 4.229(1.1); 4.083(0.3); 3.902(4.7); 3.535(1.5); 3.325(176.3); 3.079(1.6); 3.055(3.6); 3.035(3.2); 3.010(1.5); 2.891(0.8); 2.732(0.6); 2.704(0.4);
2.680(0.8); 2.676(1.5); 2.671(2.0); 2.667(1.5); 2.543(59.2); 2.524(5.8); 2.511(134.3); 2.507(264.2); 2.502(340.3); 2.498(240.2); 2.493(112.1); 2.377(0.5); 2.338(0.9); 2.333(1.6);
2.329(2.0); 2.324(1.5); 2.281(1.7); 2.149(0.9); 2.134(2.2); 2.116(3.7); 2.102(4.7); 2.082(4.0); 2.065(2.2); 2.033(0.7); 2.015(0.5); 1.909(0.4); 1.828(1.4); 1.806(4.3); 1.789(5.6);
1.786(5.4); 1.775(3.9); 1.770(4.9); 1.757(4.4); 1.744(1.9); 1.732(3.3); 1.717(2.3); 1.712(2.5); 1.706(2.7); 1.685(1.8); 1.661(0.9); 1.649(0.5); 1.628(1.3); 1.608(2.9); 1.597(1.6);
1.587(2.9); 1.577(2.8); 1.567(1.3); 1.556(2.5); 1.535(0.9); 1.259(0.3); 1.235(0.6); 0.008(0.7); 0.000(18.8); −0.008(0.5)
Example 103: ¹H-NMR(400.0 MHz. DMSO): δ = 8.591(1.2); 8.569(1.2); 8.409(1.6); 8.399(1.7); 7.689(1.6); 7.669(1.8); 7.644(0.4); 7.627(0.6); 7.616(0.5); 7.598(0.6); 7.576(0.4);
7.567(0.5); 7.557(0.4); 7.549(0.4); 7.414(1.8); 7.402(2.4); 7.395(1.9); 7.383(2.3); 7.378(1.0); 7.372(0.9); 7.367(0.8); 7.352(0.8); 7.347(0.9); 7.343(0.8); 7.321(1.6); 7.315(0.8);
7.299(1.0); 7.294(1.5); 7.272(0.8); 7.144(0.8); 7.138(0.8); 7.133(0.9); 7.128(0.9); 7.122(0.7); 7.116(0.7); 7.111(0.7); 4.358(0.9); 4.335(1.2); 4.313(0.9); 3.902(0.9); 3.333(242.8);
3.293(0.3); 3.175(0.5); 3.149(1.1); 3.129(1.0); 3.104(0.4); 2.677(0.4); 2.672(0.5); 2.668(0.4); 2.507(65.2); 2.503(82.2); 2.499(59.0); 2.336(16.0); 2.128(0.5); 2.115(0.9); 2.100(1.2);
2.082(1.2); 2.070(0.8); 2.063(0.7); 2.053(0.4); 2.041(0.3); 1.826(0.4); 1.804(1.3); 1.788(1.5); 1.783(1.6); 1.767(1.5); 1.754(1.2); 1.738(0.4); 1.726(1.0); 1.710(0.7); 1.700(0.7);
1.674(1.0); 1.664(0.5); 1.651(0.8); 1.643(0.9); 1.630(0.3); 1.621(0.7); 0.000(3.0)
Example 104: ¹H-NMR(400.0 MHz. DMSO): δ = 9.067(1.2); 9.049(4.5); 8.722(1.4); 8.677(0.8); 8.656(0.8); 8.566(5.1); 7.627(0.5); 7.623(0.4); 7.614(0.4); 7.597(0.5); 7.566(0.4);
7.432(0.6); 7.427(0.6); 7.412(0.6); 7.407(0.6); 7.401(0.6); 7.396(0.6); 7.382(1.0); 7.376(0.6); 7.361(1.2); 7.355(0.6); 7.340(0.8); 7.334(1.2); 7.312(0.7); 7.165(0.6); 7.159(0.6);
7.153(0.6); 7.149(0.6); 7.144(0.5); 7.138(0.5); 7.133(0.5); 4.328(0.7); 4.305(0.9); 4.283(0.7); 3.902(0.8); 3.324(75.3); 3.019(0.3); 2.994(0.8); 2.972(0.6); 2.676(0.3); 2.671(0.4);
2.560(4.9); 2.524(1.2); 2.511(27.0); 2.507(54.2); 2.502(70.2); 2.497(49.4); 2.493(23.0); 2.333(0.4); 2.329(0.5); 2.324(0.4); 2.311(16.0); 2.155(0.6); 2.149(0.4); 2.142(0.4); 2.137(0.5);
2.123(0.5); 2.104(0.6); 2.094(0.6); 2.075(0.6); 1.828(0.5); 1.822(0.5); 1.804(0.8); 1.790(1.1); 1.782(1.0); 1.773(0.9); 1.759(0.6); 1.741(0.7); 1.722(0.4); 1.635(0.5); 1.625(0.3);
1.615(0.5); 1.604(0.5); 1.585(0.4); 0.000(4.3)
Example 105: ¹H-NMR(400.0 MHz. DMSO): δ = 8.389(6.1); 8.369(6.1); 8.315(1.9); 7.647(1.6); 7.644(1.7); 7.640(1.0); 7.631(1.7); 7.627(3.0); 7.623(2.7); 7.614(2.3); 7.610(1.6);
7.603(0.7); 7.597(2.7); 7.574(1.9); 7.566(2.3); 7.559(1.5); 7.556(1.6); 7.548(2.1); 7.538(0.8); 7.530(0.9); 7.527(0.7); 7.510(2.8); 7.505(3.3); 7.497(3.2); 7.492(6.7); 7.487(5.3);
7.485(4.6); 7.478(4.6); 7.476(4.6); 7.474(5.5); 7.471(7.2); 7.466(4.6); 7.457(3.7); 7.453(5.0); 7.450(6.1); 7.445(5.4); 7.431(10.8); 7.427(10.1); 7.412(8.1); 7.408(6.2); 7.398(4.9);
7.393(5.1); 7.378(5.0); 7.373(5.5); 7.367(5.6); 7.363(9.2); 7.347(5.6); 7.342(15.2); 7.336(5.8); 7.320(6.6); 7.314(10.5); 7.303(0.7); 7.293(5.8); 7.261(8.1); 7.248(9.7); 7.246(10.8);
7.242(7.4); 7.240(8.4); 7.237(9.5); 7.234(9.7); 7.229(16.0); 7.228(13.8); 7.216(6.5); 7.213(7.9); 7.211(9.6); 7.208(6.1); 7.151(4.8); 7.144(4.8); 7.140(5.0); 7.134(5.1); 7.129(4.1);
7.124(3.9); 7.119(3.8); 4.310(1.7); 4.289(5.6); 4.266(7.1); 4.244(5.7); 4.223(1.8); 4.083(0.6); 3.902(3.0); 3.507(0.3); 3.409(0.4); 3.399(0.5); 3.392(0.5); 3.384(0.6); 3.361(1.7);
3.354(2.3); 3.350(2.3); 3.326(934.4); 3.303(2.1); 3.282(0.5); 3.270(0.4); 3.262(0.4); 3.081(2.8); 3.057(7.0); 3.033(4.9); 3.012(2.0); 2.680(1.3); 2.676(2.4); 2.671(3.3); 2.667(2.4);
2.662(1.2); 2.541(2.4); 2.524(8.7); 2.520(13.7); 2.511(202.8); 2.507(412.8); 2.502(540.8); 2.497(385.1); 2.493(181.6); 2.452(0.5); 2.338(1.1); 2.333(2.3); 2.329(3.2); 2.324(2.4);
2.320(1.1); 2.311(0.7); 2.169(1.1); 2.156(2.3); 2.149(2.4); 2.136(4.7); 2.121(4.1); 2.104(4.4); 2.094(3.8); 2.083(5.7); 2.063(5.1); 2.035(2.1); 1.824(1.5); 1.815(2.3); 1.806(3.5);
1.799(5.7); 1.782(10.6); 1.774(9.4); 1.767(8.8); 1.762(8.8); 1.752(8.1); 1.741(4.3); 1.734(3.0); 1.726(5.2); 1.707(2.7); 1.702(1.7); 1.683(1.1); 1.639(1.9); 1.618(4.3); 1.608(2.7);
1.598(4.2); 1.587(4.0); 1.577(2.2); 1.567(3.3); 1.545(1.4); 1.258(0.3); 1.235(0.6); 0.008(1.0); 0.000(32.5); −0.009(0.9)
Example 106: ¹H-NMR(400.0 MHz. DMSO): δ = 8.625(0.3); 8.580(8.4); 8.559(8.3); 8.319(9.6); 8.315(12.8); 8.312(10.6); 8.307(10.5); 8.305(11.4); 8.303(11.3); 8.300(9.5);
8.016(8.2); 8.011(8.1); 7.998(9.8); 7.992(16.0); 7.987(8.3); 7.974(9.0); 7.969(8.2); 7.647(2.6); 7.644(2.7); 7.632(2.7); 7.627(4.8); 7.623(4.1); 7.614(3.7); 7.603(1.2); 7.597(4.4);
7.574(3.1); 7.566(3.5); 7.556(2.7); 7.551(3.0); 7.548(3.1); 7.538(1.3); 7.530(1.3); 7.527(0.9); 7.433(9.4); 7.428(9.6); 7.420(9.5); 7.415(13.6); 7.409(10.0); 7.402(12.2); 7.397(11.7);
7.384(6.7); 7.379(7.2); 7.373(7.2); 7.367(11.4); 7.354(7.0); 7.345(15.5); 7.340(7.2); 7.324(8.6); 7.318(13.8); 7.297(7.6); 7.151(6.3); 7.145(6.5); 7.140(6.6); 7.135(6.6); 7.129(5.4);
7.124(5.1); 7.119(4.9); 4.307(2.3); 4.286(7.5); 4.265(9.3); 4.263(9.4); 4.242(7.8); 4.221(2.4); 4.083(1.1); 3.903(9.0); 3.508(0.5); 3.386(0.6); 3.323(741.5); 3.292(0.6); 3.284(0.4);
3.276(0.3); 3.175(1.7); 3.162(1.7); 3.068(3.7); 3.044(8.8); 3.020(6.4); 2.999(2.6); 2.849(0.4); 2.694(0.5); 2.680(1.6); 2.676(3.4); 2.671(4.6); 2.666(3.3); 2.662(1.6); 2.632(0.4);
2.623(0.5); 2.541(3.3); 2.524(13.6); 2.511(289.5); 2.507(570.5); 2.502(738.3); 2.497(520.8); 2.493(243.0); 2.333(3.2); 2.329(4.5); 2.324(3.1); 2.320(1.5); 2.180(1.5); 2.166(2.9);
2.160(3.1); 2.146(5.9); 2.132(5.2); 2.114(5.9); 2.102(4.9); 2.090(6.8); 2.071(6.7); 2.044(2.8); 1.858(0.4); 1.833(2.2); 1.822(3.3); 1.815(4.6); 1.808(6.7); 1.804(6.8); 1.790(12.2);
1.782(12.2); 1.776(11.5); 1.771(10.6); 1.765(10.5); 1.761(10.8); 1.751(6.0); 1.742(3.9); 1.735(7.0); 1.716(3.7); 1.711(2.4); 1.692(1.5); 1.662(0.5); 1.644(2.5); 1.623(5.3); 1.613(3.5);
1.603(5.4); 1.592(5.0); 1.583(2.9); 1.572(4.0); 1.552(1.9); 1.299(0.4); 1.275(0.3); 1.259(0.6); 1.250(0.5); 1.236(0.9); 1.225(0.5); 0.008(1.7); 0.000(44.7); −0.009(1.3)
Example 107: ¹H-NMR(400.0 MHz. DMSO): δ = 8.719(5.1); 8.698(5.1); 8.649(16.0); 8.646(15.7); 8.493(9.9); 8.490(9.3); 8.481(10.1); 8.478(9.4); 8.315(0.8); 7.647(1.2); 7.644(1.3);
7.640(0.7); 7.631(1.3); 7.627(2.2); 7.623(1.9); 7.614(1.7); 7.597(2.1); 7.574(1.4); 7.566(1.6); 7.559(1.1); 7.557(1.2); 7.551(1.4); 7.548(1.4); 7.538(0.5); 7.530(0.6); 7.436(7.8);
7.424(9.3); 7.422(9.8); 7.409(8.0); 7.403(3.9); 7.397(3.9); 7.383(3.8); 7.377(4.2); 7.372(7.5); 7.366(4.1); 7.351(10.8); 7.345(6.3); 7.329(4.9); 7.323(7.9); 7.302(4.3); 7.149(3.6);
7.143(3.7); 7.138(3.8); 7.133(3.8); 7.127(3.1); 7.122(3.0); 7.117(2.9); 4.306(1.3); 4.286(4.4); 4.264(5.5); 4.241(4.6); 4.220(1.4); 4.083(0.4); 3.903(4.6); 3.378(0.3); 3.325(477.3);
3.293(0.7); 3.284(0.5); 3.053(2.0); 3.028(5.1); 3.004(3.6); 2.984(1.4); 2.675(1.6); 2.671(2.3); 2.667(1.6); 2.549(0.4); 2.541(1.2); 2.524(6.1); 2.511(145.1); 2.507(291.7); 2.502(379.2);
2.497(269.0); 2.493(126.3); 2.338(0.9); 2.333(1.7); 2.329(2.3); 2.324(1.7); 2.185(0.9); 2.171(1.7); 2.165(1.7); 2.151(3.5); 2.138(2.8); 2.133(3.0); 2.119(3.0); 2.100(3.7); 2.088(3.9);
2.069(4.2); 2.043(1.8); 1.835(1.2); 1.823(2.0); 1.817(2.6); 1.811(3.2); 1.804(3.9); 1.794(5.8); 1.788(6.3); 1.781(9.2); 1.772(5.8); 1.766(8.1); 1.755(4.4); 1.740(4.0); 1.721(2.0);
1.716(1.4); 1.697(0.9); 1.643(1.5); 1.622(3.0); 1.612(2.1); 1.602(2.9); 1.591(2.8); 1.582(1.6); 1.571(2.3); 1.550(1.0); 1.236(0.5); 0.008(0.7); 0.000(21.2); −0.008(0.6)
Example 108: ¹H-NMR(400.0 MHz. DMSO): δ = 8.518(1.1); 8.496(1.1); 8.152(5.8); 7.644(0.3); 7.627(0.7); 7.624(0.5); 7.615(0.5); 7.598(0.6); 7.574(0.4); 7.567(0.5); 7.557(0.3);
7.551(0.4); 7.548(0.4); 7.388(0.6); 7.383(0.6); 7.369(0.7); 7.363(0.7); 7.358(0.7); 7.353(0.7); 7.339(1.1); 7.333(0.7); 7.318(1.3); 7.312(0.7); 7.297(0.8); 7.291(1.3); 7.270(0.7);
7.125(0.7); 7.119(0.7); 7.115(0.7); 7.109(0.7); 7.104(0.6); 7.098(0.5); 7.093(0.5); 4.282(0.7); 4.260(1.0); 4.237(0.8); 3.903(0.9); 3.324(61.6); 3.176(0.6); 3.162(0.6); 3.112(0.4);
3.087(0.9); 3.066(0.8); 3.042(0.4); 2.676(0.4); 2.671(0.5); 2.667(0.4); 2.630(16.0); 2.525(1.2); 2.511(29.2); 2.507(58.1); 2.502(75.0); 2.498(53.4); 2.493(25.4); 2.334(0.3); 2.329(0.5);
2.129(0.6); 2.112(0.9); 2.097(1.2); 2.081(1.0); 2.062(0.4); 1.827(0.3); 1.806(1.0); 1.789(1.3); 1.770(1.2); 1.757(0.9); 1.747(0.4); 1.735(0.7); 1.719(0.5); 1.713(0.5); 1.709(0.6);
1.688(0.4); 1.638(0.6); 1.627(0.4); 1.617(0.6); 1.607(0.7); 1.586(0.6); 0.000(3.9)
Example 109: ¹H-NMR(400.0 MHz. DMSO): δ = 8.851(0.7); 8.831(0.7); 7.627(0.3); 7.415(0.5); 7.410(0.5); 7.395(0.5); 7.390(0.5); 7.384(0.5); 7.378(0.8); 7.364(0.5); 7.356(1.1);
7.350(0.5); 7.334(0.6); 7.328(1.0); 7.307(0.5); 7.141(0.5); 7.135(0.5); 7.130(0.5); 7.125(0.5); 7.120(0.4); 7.114(0.4); 7.109(0.4); 4.306(0.6); 4.282(0.7); 4.261(0.6); 3.903(0.6);
3.325(66.0); 3.035(0.7); 3.013(0.5); 2.607(16.0); 2.525(0.9); 2.511(19.7); 2.507(39.3); 2.502(50.7); 2.498(36.2); 2.493(17.2); 2.147(0.5); 2.128(0.4); 2.115(0.4); 2.098(0.4); 2.087(0.5);
2.069(0.5); 1.820(0.4); 1.815(0.5); 1.808(0.5); 1.802(0.5); 1.796(0.8); 1.792(0.8); 1.786(1.0); 1.776(0.9); 1.762(0.7); 1.751(0.4); 1.736(0.5); 1.642(0.4); 1.621(0.4); 1.610(0.4);
0.000(2.7)
Example 110: ¹H-NMR(400.0 MHz. DMSO): δ = 9.915(16.0); 8.951(2.1); 8.947(2.2); 8.939(2.2); 8.935(2.2); 8.909(7.5); 8.905(7.9); 8.897(8.0); 8.893(7.6); 8.882(0.3); 8.316(1.0);
8.248(2.1); 8.244(2.2); 8.229(2.3); 8.225(2.2); 8.202(7.5); 8.199(7.5); 8.183(8.5); 8.179(7.9); 7.812(0.7); 7.770(2.2); 7.758(2.2); 7.751(2.1); 7.739(2.1); 7.725(7.8); 7.713(7.8);
7.706(7.3); 7.693(7.2); 7.647(1.6); 7.644(1.7); 7.628(3.1); 7.624(2.6); 7.615(2.3); 7.603(0.7); 7.598(2.6); 7.575(1.8); 7.567(2.2); 7.560(1.5); 7.557(1.7); 7.549(2.0); 7.538(0.8);
7.531(0.9); 7.433(0.8); 7.428(0.9); 7.414(0.9); 7.409(1.0); 7.403(1.1); 7.392(2.9); 7.387(3.1); 7.372(3.0); 7.367(3.1); 7.361(3.0); 7.356(2.8); 7.342(2.7); 7.336(2.7); 7.294(0.8);
7.272(1.9); 7.268(3.1); 7.246(6.6); 7.241(3.1); 7.225(4.1); 7.219(5.6); 7.198(3.0); 7.076(0.8); 7.065(0.8); 7.040(2.9); 7.034(2.9); 7.029(3.0); 7.024(2.9); 7.020(2.5); 7.013(2.4);
7.008(2.2); 4.898(1.8); 4.876(4.6); 4.850(4.9); 4.827(2.2); 4.657(0.5); 4.634(1.1); 4.611(1.2); 4.588(0.6); 4.084(0.5); 4.034(1.6); 4.008(3.5); 3.989(2.8); 3.981(2.1); 3.963(1.3);
3.949(1.5); 3.903(8.5); 3.714(0.5); 3.688(1.0); 3.668(0.9); 3.645(0.4); 3.388(0.4); 3.376(0.6); 3.368(1.0); 3.352(1.9); 3.326(693.0); 3.305(1.8); 3.280(0.3); 3.061(0.8); 2.882(0.8);
2.677(1.8); 2.672(2.6); 2.667(1.9); 2.542(1.8); 2.525(6.9); 2.512(161.5); 2.507(320.5); 2.503(415.0); 2.498(294.1); 2.494(138.1); 2.448(0.4); 2.427(0.4); 2.408(0.7); 2.394(1.2);
2.385(1.7); 2.377(1.4); 2.370(1.7); 2.362(2.4); 2.353(1.9); 2.347(1.6); 2.339(2.6); 2.334(2.7); 2.330(3.6); 2.325(2.1); 2.320(1.3); 2.249(0.4); 2.240(0.5); 2.229(0.6); 2.218(0.8);
2.207(0.9); 2.189(1.9); 2.176(2.6); 2.170(3.2); 2.158(2.2); 2.148(3.6); 2.140(2.8); 2.129(1.8); 2.121(2.0); 2.093(2.1); 2.087(2.0); 2.072(2.8); 2.064(2.5); 2.050(2.2); 2.041(2.6);
2.030(1.3); 2.018(2.3); 2.009(1.5); 1.991(2.2); 1.986(2.4); 1.973(2.4); 1.949(1.5); 1.944(1.3); 1.930(1.0); 1.922(1.2); 1.908(2.5); 1.901(2.4); 1.893(2.4); 1.887(3.0); 1.880(2.2);
1.872(2.4); 1.863(3.0); 1.839(4.0); 1.817(3.4); 1.811(2.5); 1.789(2.2); 1.767(0.9); 1.713(0.4); 1.235(0.7); 0.008(0.8); 0.000(21.3); −0.009(0.6)
Example 111: ¹H-NMR(400.0 MHz. DMSO): δ = 7.480(1.0); 7.458(1.0); 7.263(0.6); 7.257(0.6); 7.251(0.7); 7.242(1.3); 7.236(1.3); 7.232(0.8); 7.225(0.8); 7.220(1.4); 7.215(1.3);
7.205(0.6); 7.200(0.6); 7.193(0.7); 7.033(0.6); 7.027(0.6); 7.022(0.7); 7.017(0.7); 7.012(0.6); 7.006(0.5); 7.000(0.5); 6.776(1.4); 6.771(2.2); 6.766(1.5); 6.450(1.6); 6.446(1.6);
6.441(1.7); 6.437(1.5); 5.912(1.7); 5.905(1.8); 5.902(1.8); 5.896(1.6); 4.556(0.6); 4.538(0.8); 4.519(0.6); 3.902(0.7); 3.614(16.0); 3.322(57.9); 3.294(0.4); 3.272(1.0); 3.252(1.0);
3.232(0.4); 2.670(0.4); 2.524(1.1); 2.510(24.5); 2.506(48.3); 2.502(62.3); 2.497(44.4); 2.493(21.2); 2.328(0.4); 2.063(0.3); 2.054(0.6); 2.044(0.6); 2.035(0.6); 2.026(0.5); 2.003(0.3);
1.997(0.4); 1.988(0.5); 1.979(1.2); 1.975(1.0); 1.968(0.9); 1.958(2.4); 1.938(1.6); 1.924(0.4); 1.913(0.5); 1.904(0.5); 1.895(0.5); 1.885(0.4); 1.726(0.4); 1.720(0.5); 1.704(0.6);
1.700(0.4); 1.693(0.3); 1.686(0.5); 1.672(0.4); 1.667(0.3); 1.615(0.4); 1.595(0.7); 1.587(0.5); 1.573(0.5); 1.565(0.6); 1.544(0.3); 0.000(3.0)
Example 112: ¹H-NMR(400.0 MHz. DMSO): δ = 8.661(4.4); 8.660(4.4); 8.315(0.6); 7.765(1.0); 7.743(1.0); 7.278(0.7); 7.256(1.5); 7.250(0.8); 7.235(1.4); 7.229(1.8); 7.217(0.8);
7.211(0.9); 7.207(1.3); 7.200(0.7); 7.186(0.7); 7.180(0.7); 7.021(0.7); 7.015(0.7); 7.009(0.7); 7.004(0.8); 6.999(0.6); 6.994(0.6); 6.989(0.6); 4.570(0.6); 4.552(0.8); 4.549(0.7);
4.534(0.6); 3.902(1.0); 3.327(162.9); 3.300(1.1); 3.278(1.0); 3.259(0.4); 2.676(0.5); 2.671(0.6); 2.667(0.5); 2.541(0.4); 2.524(1.7); 2.511(39.6); 2.507(79.6); 2.502(103.6); 2.497(73.3);
2.493(34.2); 2.405(16.0); 2.333(0.5); 2.329(0.6); 2.324(0.5); 2.098(0.4); 2.094(0.4); 2.085(0.6); 2.075(0.7); 2.066(0.6); 2.058(0.5); 2.002(0.7); 1.993(0.9); 1.983(1.0); 1.975(1.2);
1.966(0.8); 1.952(1.2); 1.945(1.2); 1.933(1.1); 1.924(0.8); 1.914(0.6); 1.906(0.6); 1.896(0.4); 1.705(0.4); 1.699(0.5); 1.684(0.6); 1.678(0.5); 1.665(0.7); 1.651(0.9); 1.648(0.9);
1.634(0.9); 1.626(0.5); 1.613(0.5); 0.000(4.9)
Example 113: ¹H-NMR(400.0 MHz. DMSO): δ = 10.872(0.4); 7.739(5.5); 7.290(0.7); 7.269(1.4); 7.263(0.8); 7.247(0.9); 7.242(1.4); 7.228(0.7); 7.221(1.0); 7.209(0.7); 7.203(0.8);
7.198(0.7); 7.192(0.7); 7.178(0.7); 7.173(0.7); 7.023(0.7); 7.017(0.7); 7.012(0.7); 7.007(0.8); 7.001(0.7); 6.996(0.6); 6.992(0.6); 6.909(0.5); 6.890(0.5); 4.534(0.7); 4.519(0.8);
4.513(0.8); 4.497(0.7); 3.902(1.7); 3.586(16.0); 3.327(51.4); 3.295(1.1); 3.276(1.1); 3.257(0.4); 3.169(0.5); 2.676(0.4); 2.671(0.5); 2.667(0.4); 2.541(0.3); 2.524(1.6); 2.511(31.9);
2.507(62.4); 2.502(80.2); 2.497(56.9); 2.493(26.8); 2.333(0.3); 2.329(0.5); 2.104(0.3); 2.081(0.8); 2.060(0.9); 2.039(0.9); 2.034(0.8); 2.026(0.7); 2.017(0.4); 2.011(0.5); 1.994(0.4);
1.901(0.3); 1.887(0.8); 1.871(1.1); 1.864(1.2); 1.858(1.1); 1.850(0.9); 1.845(0.8); 1.835(0.3); 1.828(0.6); 1.710(0.5); 1.703(0.3); 1.699(0.4); 1.692(0.5); 1.680(0.5); 1.670(0.5);
1.659(0.4); 1.639(0.4); 1.626(0.5); 1.621(0.6); 1.607(0.6); 1.589(0.6); 1.575(0.4); 0.000(6.3)
Example 114: ¹H-NMR(400.0 MHz. DMSO): δ = 8.315(0.4); 7.861(1.5); 7.839(1.5); 7.452(5.4); 7.447(2.0); 7.435(2.2); 7.431(6.5); 7.271(0.4); 7.254(1.2); 7.249(1.2); 7.244(1.2);
7.232(2.3); 7.227(1.7); 7.218(1.2); 7.211(2.6); 7.205(7.4); 7.184(5.7); 7.032(1.0); 7.026(1.0); 7.021(1.0); 7.016(1.0); 7.011(0.9); 7.005(0.8); 7.001(0.7); 4.639(0.4); 4.621(1.0);
4.602(1.3); 4.584(1.0); 4.565(0.4); 3.902(1.4); 3.325(215.1); 3.302(1.9); 3.281(0.7); 2.680(0.4); 2.675(0.7); 2.671(0.9); 2.666(0.7); 2.662(0.3); 2.629(1.4); 2.610(4.5); 2.591(4.8);
2.572(1.8); 2.541(0.6); 2.524(2.5); 2.511(55.5); 2.506(111.6); 2.502(145.2); 2.497(102.7); 2.493(47.8); 2.333(0.7); 2.328(0.9); 2.324(0.6); 2.105(0.4); 2.096(0.4); 2.086(0.5); 2.082(0.5);
2.077(0.7); 2.073(0.9); 2.064(1.0); 2.055(0.9); 2.049(0.7); 2.045(0.7); 2.031(0.5); 2.023(1.1); 2.015(0.7); 2.005(1.9); 2.000(1.6); 1.995(1.4); 1.984(3.5); 1.974(0.8); 1.963(2.2);
1.954(0.7); 1.944(0.5); 1.933(0.8); 1.924(0.7); 1.914(0.8); 1.905(0.6); 1.897(0.5); 1.886(0.4); 1.777(0.5); 1.761(0.5); 1.756(0.9); 1.740(1.0); 1.735(0.6); 1.728(0.5); 1.722(0.8);
1.707(0.6); 1.688(0.4); 1.639(0.7); 1.632(0.4); 1.619(1.1); 1.610(0.7); 1.597(0.7); 1.589(0.9); 1.567(0.5); 1.174(7.3); 1.155(16.0); 1.136(7.1); 0.008(1.4); 0.000(42.5); −0.009(1.4)
Example 115: ¹H-NMR(400.0 MHz. DMSO): δ = 8.315(0.4); 7.799(1.9); 7.777(1.9); 7.577(2.9); 7.572(3.1); 7.558(3.1); 7.553(3.2); 7.412(1.5); 7.408(1.5); 7.394(2.2); 7.391(2.6);
7.387(2.0); 7.373(1.9); 7.369(1.7); 7.316(1.2); 7.295(2.5); 7.289(1.3); 7.272(2.6); 7.267(2.8); 7.256(1.3); 7.251(1.5); 7.246(2.6); 7.241(1.4); 7.226(1.2); 7.220(1.2); 7.056(5.3);
7.035(4.5); 6.979(2.2); 6.977(2.1); 6.960(3.8); 6.941(1.9); 6.939(1.8); 4.690(0.5); 4.673(1.1); 4.661(1.3); 4.653(1.3); 4.640(1.1); 4.624(0.4); 4.068(1.8); 4.051(5.8); 4.033(6.0);
4.016(2.0); 3.902(1.6); 3.362(1.0); 3.327(303.5); 2.676(0.7); 2.671(0.9); 2.667(0.7); 2.541(0.5); 2.511(62.9); 2.507(121.0); 2.502(153.8); 2.498(110.6); 2.333(0.7); 2.329(0.9);
2.325(0.7); 2.147(0.6); 2.126(1.4); 2.106(1.6); 2.094(1.1); 2.084(1.7); 2.077(1.5); 2.065(1.1); 2.056(0.9); 2.051(0.8); 2.034(0.7); 1.940(0.3); 1.930(0.6); 1.919(1.5); 1.900(2.2);
1.890(2.4); 1.883(1.9); 1.861(1.1); 1.837(0.5); 1.773(0.4); 1.758(0.5); 1.746(0.7); 1.742(0.7); 1.736(0.7); 1.730(0.9); 1.719(1.1); 1.712(1.5); 1.695(1.6); 1.682(1.2); 1.667(1.0);
1.655(0.7); 1.634(0.3); 1.248(7.7); 1.231(16.0); 1.214(7.4); 0.000(4.1)
Example 116: ¹H-NMR(400.0 MHz. DMSO): δ = 7.331(0.5); 7.309(1.1); 7.304(1.0); 7.298(0.6); 7.288(0.8); 7.282(1.5); 7.272(0.6); 7.267(0.6); 7.261(0.7); 7.252(0.5); 7.247(0.5);
7.084(0.5); 7.078(0.6); 7.073(0.6); 7.068(0.6); 7.063(0.5); 7.058(0.5); 7.006(0.8); 6.985(0.8); 4.594(0.5); 4.583(0.5); 4.578(0.5); 4.574(0.5); 4.561(0.5); 3.902(0.6); 3.596(16.0);
3.463(13.5); 3.326(152.5); 3.304(1.1); 3.286(0.7); 3.280(0.7); 3.261(0.4); 2.675(0.3); 2.671(0.5); 2.666(0.4); 2.524(1.2); 2.511(30.1); 2.506(59.0); 2.502(75.5); 2.497(53.6); 2.493(25.4);
2.333(0.3); 2.329(0.5); 2.324(0.3); 2.088(0.4); 2.079(0.4); 2.068(0.5); 2.061(0.6); 2.051(0.5); 2.038(0.6); 2.024(0.4); 2.013(0.6); 2.007(0.5); 1.993(0.4); 1.959(0.6); 1.929(13.4);
1.914(0.6); 1.888(0.6); 1.879(0.5); 1.858(0.3); 1.693(0.4); 1.678(0.6); 1.661(1.0); 1.646(0.8); 1.641(0.8); 1.627(0.3); 1.622(0.4); 0.000(8.5)
Example 117: ¹H-NMR(400.0 MHz. DMSO): δ = 8.230(1.1); 8.207(1.1); 7.349(0.7); 7.327(1.4); 7.321(0.7); 7.306(0.9); 7.300(1.4); 7.278(1.1); 7.269(1.8); 7.250(4.9); 7.234(5.5);
7.225(0.7); 7.219(0.8); 7.215(1.8); 7.079(0.7); 7.074(0.7); 7.068(0.7); 7.064(0.7); 7.058(0.6); 7.052(0.6); 7.048(0.5); 4.619(0.3); 4.613(0.6); 4.602(0.6); 4.596(0.6); 4.591(0.6);
4.585(0.5); 4.580(0.5); 4.574(0.3); 3.902(0.9); 3.327(207.6); 3.288(0.8); 3.269(0.5); 2.675(0.5); 2.671(0.7); 2.666(0.5); 2.541(0.5); 2.524(2.1); 2.511(45.5); 2.506(88.9); 2.502(114.2);
2.497(79.8); 2.493(36.8); 2.427(16.0); 2.333(0.5); 2.329(0.7); 2.324(0.5); 2.103(0.4); 2.093(0.5); 2.084(0.6); 2.075(0.6); 2.066(0.5); 1.997(0.5); 1.989(0.5); 1.978(0.8); 1.971(0.8);
1.959(0.5); 1.948(0.7); 1.943(0.6); 1.922(0.7); 1.916(0.5); 1.901(0.7); 1.894(0.7); 1.883(0.8); 1.877(0.7); 1.867(0.4); 1.856(0.4); 1.847(0.4); 1.704(0.4); 1.698(0.5); 1.686(0.7);
1.679(0.5); 1.666(0.8); 1.655(1.3); 1.641(0.9); 1.621(0.4); 0.008(0.3); 0.000(8.4)
Example 118: ¹H-NMR(400.0 MHz. DMSO): δ = 8.315(0.8); 8.016(0.5); 8.010(0.6); 7.995(0.7); 7.991(1.0); 7.982(0.7); 7.967(0.7); 7.962(0.8); 7.955(0.7); 7.946(0.3); 7.926(2.3);
7.882(3.8); 7.873(0.7); 7.860(0.7); 7.856(0.7); 7.852(0.7); 7.845(0.7); 7.817(5.4); 7.788(0.6); 7.638(0.8); 7.614(1.1); 7.608(0.7); 7.592(0.8); 7.587(1.0); 7.574(0.5); 7.566(0.7);
7.561(0.3); 7.342(0.8); 7.320(1.4); 7.315(0.8); 7.299(0.9); 7.293(1.3); 7.272(0.7); 7.241(0.6); 7.236(0.7); 7.221(0.7); 7.216(0.7); 7.210(0.7); 7.205(0.7); 7.190(0.6); 7.185(0.6);
7.092(0.5); 7.033(0.7); 7.028(0.7); 7.022(0.7); 7.018(0.7); 7.007(0.6); 6.458(0.5); 6.429(1.2); 6.407(1.2); 5.651(0.4); 4.894(0.5); 4.879(1.0); 4.864(1.5); 4.848(1.6); 4.833(1.2);
4.817(0.7); 4.788(0.5); 4.773(1.3); 4.757(1.7); 4.742(1.3); 4.727(0.5); 4.579(0.7); 4.572(0.4); 4.564(0.8); 4.557(0.7); 4.549(0.4); 4.543(0.7); 3.902(2.6); 3.734(0.4); 3.729(0.5);
3.702(1.4); 3.693(2.8); 3.684(14.4); 3.643(2.4); 3.636(16.0); 3.327(507.5); 3.278(0.8); 3.201(1.0); 3.100(0.5); 3.080(1.7); 3.062(2.7); 3.043(1.4); 2.676(1.6); 2.671(2.1); 2.667(1.5);
2.541(1.0); 2.524(5.5); 2.511(137.7); 2.507(270.0); 2.502(348.2); 2.497(247.5); 2.493(117.4); 2.427(0.4); 2.417(0.8); 2.337(0.9); 2.333(1.7); 2.329(2.3); 2.324(1.7); 2.320(0.9);
2.315(1.2); 2.296(2.4); 2.278(1.3); 2.188(0.4); 2.173(0.6); 2.166(0.9); 2.155(0.5); 2.146(0.9); 2.124(0.5); 2.036(0.4); 2.029(0.5); 2.014(0.6); 1.998(0.6); 1.984(0.6); 1.969(0.4);
1.896(0.5); 1.882(0.7); 1.875(0.8); 1.866(1.0); 1.854(1.6); 1.836(2.1); 1.818(1.9); 1.807(0.7); 1.799(1.7); 1.788(0.5); 1.781(0.7); 1.773(0.7); 1.767(0.6); 1.747(0.6); 1.739(0.5);
1.736(0.5); 1.730(0.5); 1.724(0.5); 1.714(0.4); 1.635(1.0); 1.628(0.9); 1.622(1.0); 1.603(1.0); 1.590(0.6); 1.585(0.5); 1.570(0.3); 1.386(1.0); 1.370(1.1); 1.359(1.0); 1.344(2.3);
1.338(5.3); 1.329(14.9); 1.322(5.6); 1.314(14.0); 1.303(5.0); 1.288(3.8); 1.264(0.5); 1.259(0.4); 1.235(0.6); 1.219(8.3); 1.204(8.2); 1.153(0.3); 1.140(8.2); 1.124(8.1); 0.008(1.2);
0.000(34.0); −0.008(1.2)
Example 119: ¹H-NMR(400.0 MHz. DMSO): δ = 8.315(0.6); 7.986(3.3); 7.964(3.4); 7.459(2.5); 7.455(2.6); 7.439(4.2); 7.436(4.3); 7.420(3.5); 7.416(3.5); 7.327(2.1); 7.306(4.6);
7.300(2.4); 7.284(2.9); 7.278(4.7); 7.273(2.4); 7.267(2.3); 7.257(2.9); 7.252(2.4); 7.247(2.5); 7.240(5.1); 7.237(5.3); 7.221(8.4); 7.219(8.2); 7.202(3.8); 7.200(4.5); 7.197(6.1);
7.165(5.6); 7.145(4.8); 7.079(2.2); 7.073(2.3); 7.068(2.3); 7.062(2.3); 7.057(2.0); 7.052(1.9); 7.046(1.8); 7.011(16.0); 6.995(4.9); 6.991(4.7); 6.825(5.6); 4.624(0.8); 4.606(1.8);
4.595(2.0); 4.590(2.1); 4.585(1.9); 4.578(1.5); 4.573(1.7); 4.556(0.8); 3.902(3.5); 3.323(367.4); 3.289(2.7); 3.271(1.4); 2.675(1.2); 2.670(1.6); 2.666(1.2); 2.541(1.1); 2.510(106.4);
2.506(209.4); 2.501(271.5); 2.497(193.5); 2.492(91.6); 2.333(1.2); 2.328(1.6); 2.324(1.2); 2.109(0.9); 2.087(1.4); 2.078(1.8); 2.068(2.0); 2.060(2.1); 2.050(1.6); 2.036(1.4); 2.027(0.8);
2.005(1.8); 1.996(1.5); 1.985(2.5); 1.974(1.9); 1.966(1.5); 1.952(2.5); 1.927(2.1); 1.918(1.9); 1.906(2.4); 1.902(2.3); 1.897(2.5); 1.886(2.6); 1.876(1.3); 1.866(1.3); 1.857(1.1);
1.844(0.6); 1.835(0.5); 1.710(1.0); 1.698(1.2); 1.692(1.6); 1.679(2.4); 1.670(2.0); 1.660(2.6); 1.652(3.6); 1.640(3.4); 1.621(1.3); 1.616(1.1); 1.603(0.6); 1.594(0.8); 0.008(0.5);
0.000(13.5); −0.008(0.4)
Example 120: ¹H-NMR(400.0 MHz. DMSO): δ = 8.461(3.8); 8.437(3.8); 8.355(15.4); 8.342(16.0); 8.315(1.0); 7.598(9.5); 7.584(9.1); 7.348(2.4); 7.327(5.3); 7.321(2.7); 7.305(3.6);
7.299(7.4); 7.295(2.8); 7.278(4.8); 7.268(2.5); 7.263(2.4); 7.248(2.2); 7.243(2.2); 7.104(2.6); 7.099(2.7); 7.093(2.7); 7.088(2.6); 7.083(2.3); 7.077(2.2); 7.072(2.0); 4.739(1.0);
4.732(1.2); 4.723(2.2); 4.716(2.9); 4.707(2.2); 4.699(3.0); 4.692(2.0); 4.683(1.2); 4.677(0.9); 3.902(5.5); 3.326(634.3); 3.282(3.0); 3.265(1.7); 2.680(1.0); 2.675(2.0); 2.671(2.7);
2.666(2.0); 2.662(0.9); 2.541(1.6); 2.524(7.3); 2.511(174.2); 2.506(343.9); 2.502(444.4); 2.497(314.8); 2.493(147.6); 2.337(1.1); 2.333(2.1); 2.328(2.9); 2.324(2.0); 2.320(1.0);
2.140(0.7); 2.132(0.7); 2.123(0.8); 2.116(1.9); 2.108(1.4); 2.100(2.2); 2.094(2.3); 2.085(2.0); 2.078(2.0); 2.066(1.9); 2.047(2.6); 2.035(1.8); 2.023(2.4); 2.017(2.6); 2.003(1.4);
1.980(1.0); 1.958(2.2); 1.937(1.8); 1.929(2.4); 1.908(2.2); 1.898(1.8); 1.879(2.5); 1.873(1.7); 1.864(1.7); 1.857(1.9); 1.850(1.8); 1.840(1.7); 1.830(1.2); 1.815(1.0); 1.807(0.6);
1.747(1.4); 1.739(1.6); 1.733(2.1); 1.726(2.8); 1.712(4.3); 1.692(4.3); 1.683(2.4); 1.675(1.8); 1.668(1.4); 1.653(0.8); 1.646(0.5); 0.146(0.4); 0.008(3.6); 0.000(94.3); −0.009(3.2); −0.150
(0.4)
Example 121: ¹H-NMR(400.0 MHz. DMSO): δ = 8.499(0.4); 8.465(8.3); 8.444(8.4); 8.308(16.0); 7.351(5.2); 7.329(11.5); 7.323(5.9); 7.307(7.6); 7.302(11.1); 7.281(10.7); 7.263(6.6);
7.257(7.1); 7.252(6.8); 7.246(6.5); 7.232(6.2); 7.227(6.1); 7.077(7.0); 7.071(7.2); 7.066(7.3); 7.061(7.2); 7.056(6.2); 7.050(6.1); 7.044(5.8); 4.611(2.5); 4.599(3.4); 4.593(6.2);
4.582(6.8); 4.576(6.6); 4.572(6.6); 4.560(5.9); 4.554(3.7); 4.542(2.8); 3.898(10.9); 3.511(0.5); 3.404(1.0); 3.368(7.6); 3.332(1942.6); 3.327(1763.9); 3.226(0.6); 3.198(0.6); 3.178(1.0);
3.164(0.9); 3.135(0.3); 2.677(5.2); 2.672(7.0); 2.668(4.9); 2.543(3.5); 2.526(18.3); 2.512(439.4); 2.508(871.4); 2.503(1122.4); 2.499(789.2); 2.494(366.0); 2.411(0.6); 2.397(0.6);
2.339(2.8); 2.335(5.5); 2.330(7.3); 2.326(5.1); 2.304(0.4); 2.230(0.4); 2.162(2.9); 2.143(3.6); 2.138(3.7); 2.128(6.5); 2.119(6.6); 2.110(6.1); 2.101(5.4); 2.087(3.2); 2.035(1.3);
2.026(1.7); 2.004(6.2); 1.995(4.4); 1.983(12.5); 1.963(10.2); 1.938(7.9); 1.917(9.3); 1.909(8.2); 1.904(6.9); 1.899(7.3); 1.882(5.3); 1.874(4.1); 1.864(2.2); 1.854(1.6); 1.796(0.3);
1.758(3.3); 1.746(3.5); 1.738(5.1); 1.726(7.3); 1.713(3.9); 1.706(6.1); 1.694(5.9); 1.673(5.5); 1.666(4.0); 1.651(6.6); 1.631(5.3); 1.612(2.4); 1.604(2.7); 1.581(1.1); 1.299(0.4);
1.260(0.6); 1.236(0.7); 1.226(0.3); 0.855(0.4); 0.146(1.0); 0.008(8.0); 0.000(238.1); −0.009(7.5); −0.150(1.0)
Example 122: ¹H-NMR(400.0 MHz. DMSO): δ = 8.315(0.4); 7.945(1.8); 7.923(1.8); 7.289(1.1); 7.268(2.4); 7.262(1.3); 7.246(1.5); 7.240(2.4); 7.231(1.2); 7.226(1.2); 7.219(1.4);
7.211(1.2); 7.206(1.2); 7.200(1.2); 7.195(1.1); 7.180(1.1); 7.175(1.1); 7.039(1.2); 7.033(1.2); 7.028(1.2); 7.023(1.2); 7.018(1.0); 7.012(1.0); 7.007(1.0); 4.554(0.4); 4.542(0.6);
4.537(0.9); 4.526(1.1); 4.520(1.0); 4.515(1.1); 4.504(0.9); 4.486(0.4); 3.902(2.4); 3.757(16.0); 3.322(167.1); 3.302(0.9); 3.282(1.4); 3.259(1.2); 3.241(0.7); 2.675(0.7); 2.671(1.0);
2.666(0.7); 2.541(0.7); 2.524(3.0); 2.511(64.6); 2.506(127.4); 2.502(164.4); 2.497(116.7); 2.493(55.0); 2.333(0.7); 2.328(1.0); 2.324(0.7); 2.099(0.4); 2.089(0.7); 2.081(1.1); 2.071(1.1);
2.063(1.0); 2.052(1.0); 2.031(0.7); 2.000(0.9); 1.991(0.8); 1.980(1.5); 1.961(1.2); 1.940(1.2); 1.915(1.0); 1.902(1.3); 1.894(1.1); 1.884(1.4); 1.875(1.3); 1.865(1.1); 1.859(0.8);
1.845(0.6); 1.674(0.5); 1.657(1.2); 1.647(1.1); 1.635(1.9); 1.620(1.9); 1.610(1.1); 1.601(1.0); 1.590(0.7); 0.000(9.0)
Example 123: ¹H-NMR(400.0 MHz. DMSO): δ = 8.314(4.6); 8.019(0.8); 7.997(0.8); 7.332(0.5); 7.310(1.1); 7.304(0.6); 7.289(0.7); 7.283(1.1); 7.261(0.6); 7.249(0.5); 7.244(0.5);
7.229(0.5); 7.223(0.6); 7.218(0.5); 7.212(0.5); 7.198(0.5); 7.193(0.5); 7.039(0.5); 7.033(0.6); 7.028(0.6); 7.023(0.6); 7.017(0.5); 7.012(0.5); 7.007(0.4); 4.557(0.5); 4.543(0.6);
4.540(0.6); 4.522(0.5); 3.902(1.2); 3.378(0.4); 3.332(315.1); 3.292(1.0); 3.273(0.4); 2.676(0.5); 2.671(0.7); 2.667(0.5); 2.542(0.4); 2.525(1.9); 2.511(41.5); 2.507(82.7); 2.502(107.7);
2.498(76.4); 2.493(35.8); 2.333(0.5); 2.329(0.7); 2.325(0.5); 2.166(16.0); 2.147(0.7); 2.088(0.5); 2.078(0.5); 2.069(0.5); 2.060(0.5); 1.986(0.5); 1.976(0.7); 1.966(0.7); 1.958(0.9);
1.932(0.9); 1.911(0.6); 1.903(0.6); 1.894(0.7); 1.885(0.4); 1.875(0.4); 1.713(0.4); 1.699(0.6); 1.679(0.5); 1.666(0.4); 1.659(0.4); 1.631(0.4); 1.610(0.5); 1.589(0.4); 0.000(0.4)
Example 124: ¹H-NMR(400.0 MHz. DMSO): δ = 8.431(5.1); 8.409(5.1); 8.315(1.7); 8.001(14.1); 7.982(16.0); 7.702(10.1); 7.683(8.8); 7.374(3.0); 7.352(6.4); 7.347(3.3); 7.331(3.9);
7.325(6.2); 7.304(3.6); 7.298(3.0); 7.293(3.0); 7.278(2.9); 7.273(3.2); 7.267(3.1); 7.262(3.0); 7.247(2.7); 7.242(2.8); 7.098(3.1); 7.093(3.1); 7.087(3.2); 7.082(3.2); 7.077(2.7);
7.072(2.7); 7.066(2.5); 4.680(1.0); 4.670(1.3); 4.663(2.4); 4.653(2.9); 4.647(2.6); 4.640(2.8); 4.631(2.3); 4.624(1.5); 4.614(1.1); 3.902(4.9); 3.328(946.4); 3.327(972.0); 3.293(3.2);
3.245(0.4); 3.194(0.4); 2.680(1.2); 2.676(2.6); 2.671(3.7); 2.666(2.7); 2.662(1.2); 2.541(1.7); 2.524(8.7); 2.511(217.3); 2.507(442.4); 2.502(581.8); 2.497(415.5); 2.493(196.3);
2.338(1.4); 2.333(2.8); 2.329(3.8); 2.324(2.8); 2.166(0.4); 2.155(1.2); 2.132(1.9); 2.123(2.2); 2.112(2.6); 2.102(2.6); 2.094(2.2); 2.081(1.5); 2.063(0.8); 2.056(1.0); 2.049(1.0);
2.029(2.5); 2.019(2.2); 2.006(3.9); 1.987(1.6); 1.970(0.8); 1.959(1.2); 1.937(2.4); 1.915(2.6); 1.907(3.3); 1.890(5.0); 1.883(4.6); 1.873(3.7); 1.856(1.9); 1.835(0.9); 1.827(0.8);
1.741(1.6); 1.731(1.6); 1.725(2.0); 1.705(3.2); 1.693(5.7); 1.683(3.3); 1.677(4.5); 1.674(4.6); 1.657(2.0); 1.641(1.0); 1.634(1.0); 1.613(0.4); 1.236(0.3); 0.008(0.8); 0.000(26.4); −0.009
(0.9)
Example 125: ¹H-NMR(400.0 MHz. DMSO): δ = 8.315(0.3); 8.225(3.9); 8.168(1.5); 8.147(1.5); 7.371(1.7); 7.363(0.9); 7.348(0.9); 7.343(1.0); 7.336(1.5); 7.332(1.0); 7.314(2.4);
7.308(1.1); 7.293(1.1); 7.287(1.8); 7.265(1.0); 7.236(3.4); 7.118(0.9); 7.112(1.0); 7.100(2.4); 7.086(0.7); 4.264(1.0); 4.243(1.3); 4.220(1.0); 3.902(1.6); 3.893(16.0); 3.326(260.9);
3.071(0.5); 3.046(1.2); 3.025(1.1); 3.002(0.5); 2.676(0.6); 2.671(0.8); 2.667(0.6); 2.541(0.5); 2.524(2.5); 2.511(52.9); 2.507(103.6); 2.502(133.3); 2.497(94.1); 2.493(44.0); 2.333(0.6);
2.329(0.8); 2.324(0.6); 2.125(0.7); 2.109(1.2); 2.095(1.5); 2.074(1.1); 2.063(0.5); 2.058(0.5); 2.051(0.4); 1.816(0.4); 1.794(1.4); 1.776(1.8); 1.762(1.2); 1.757(1.5); 1.743(1.2);
1.726(0.5); 1.716(0.9); 1.699(0.6); 1.694(0.7); 1.689(0.7); 1.668(0.5); 1.601(0.3); 1.581(0.9); 1.570(0.4); 1.560(0.9); 1.550(0.9); 1.539(0.4); 1.529(0.8); 0.008(0.6); 0.000(16.0); −0.008
(0.5)
Example 126: ¹H-NMR(400.0 MHz. DMSO): δ = 8.315(0.4); 8.111(2.9); 8.092(3.2); 7.962(1.0); 7.948(1.1); 7.941(1.2); 7.927(0.9); 7.365(1.0); 7.360(0.7); 7.345(1.0); 7.339(1.1);
7.333(1.7); 7.312(2.4); 7.290(1.1); 7.285(1.7); 7.263(0.9); 7.108(1.1); 6.190(0.8); 6.176(0.9); 6.174(0.9); 6.161(0.7); 6.072(0.8); 6.058(0.9); 6.056(0.9); 6.042(0.8); 4.265(0.6);
4.257(0.7); 4.242(0.9); 4.236(0.9); 4.221(0.7); 4.213(0.7); 3.902(1.0); 3.834(16.0); 3.326(302.7); 3.051(0.4); 3.040(0.7); 3.026(0.9); 3.007(0.7); 2.676(0.7); 2.671(1.0); 2.667(0.7);
2.541(0.6); 2.524(2.9); 2.507(120.5); 2.502(153.5); 2.498(109.4); 2.494(52.1); 2.333(0.7); 2.329(0.9); 2.324(0.7); 2.103(1.1); 2.088(1.6); 2.079(1.3); 2.069(1.3); 2.060(1.1); 2.042(0.5);
1.807(0.5); 1.788(1.4); 1.768(2.0); 1.753(1.6); 1.739(1.0); 1.716(0.8); 1.706(0.6); 1.689(0.8); 1.667(0.4); 1.591(2.6); 1.574(3.2); 1.567(3.4); 1.551(3.5); 1.543(1.2); 1.532(3.2);
1.516(2.8); 1.509(3.3); 1.492(2.9); 0.008(0.6); 0.000(15.2); −0.008(0.5)
Example 127: ¹H-NMR(400.0 MHz. DMSO): δ = 7.850(5.5); 7.400(0.7); 7.395(0.7); 7.380(0.7); 7.375(0.8); 7.370(0.8); 7.364(0.7); 7.350(1.3); 7.345(0.7); 7.329(1.4); 7.323(0.7);
7.308(0.9); 7.302(1.4); 7.281(0.7); 7.132(0.7); 7.126(0.8); 7.122(0.8); 7.117(0.8); 7.111(0.7); 7.106(0.6); 7.100(0.6); 6.929(1.3); 6.909(1.4); 4.240(0.8); 4.237(0.8); 4.231(0.4);
4.223(2.5); 4.219(2.5); 4.205(2.8); 4.202(2.7); 4.193(0.4); 4.184(1.5); 4.160(1.0); 4.139(0.8); 3.903(0.8); 3.653(16.0); 3.323(70.7); 3.020(0.4); 2.996(1.1); 2.974(0.8); 2.951(0.4);
2.676(0.3); 2.671(0.5); 2.667(0.3); 2.524(1.3); 2.511(28.6); 2.506(56.2); 2.502(73.1); 2.498(53.2); 2.493(26.2); 2.333(0.3); 2.329(0.4); 2.324(0.3); 2.188(0.3); 2.183(0.4); 2.170(0.7);
2.152(0.7); 2.138(0.6); 2.084(0.5); 2.072(0.8); 2.065(0.6); 2.052(0.7); 2.045(0.6); 2.024(0.3); 1.807(0.4); 1.788(0.8); 1.775(1.2); 1.764(1.0); 1.757(1.0); 1.749(1.1); 1.740(0.8);
1.725(0.9); 1.706(0.5); 1.700(0.7); 1.680(0.4); 1.524(0.6); 1.514(0.4); 1.503(0.7); 1.493(0.6); 1.482(0.3); 1.472(0.6); 1.350(4.6); 1.333(9.5); 1.315(4.5); 0.000(3.3)
Example 128: ¹H-NMR(400.0 MHz. DMSO): δ = 8.911(1.0); 8.890(1.0); 8.307(1.5); 7.359(0.6); 7.351(0.7); 7.346(0.7); 7.337(1.4); 7.331(1.3); 7.326(0.8); 7.315(1.5); 7.310(1.3);
7.300(0.7); 7.295(0.7); 7.289(0.6); 7.113(0.7); 7.107(0.7); 7.102(0.8); 7.097(0.7); 7.092(0.7); 7.081(0.6); 4.222(0.8); 4.200(1.1); 4.178(0.8); 3.898(0.8); 3.333(173.0); 3.327(256.2);
3.005(0.4); 2.980(1.0); 2.958(0.8); 2.695(16.0); 2.677(0.6); 2.672(0.8); 2.668(0.6); 2.543(0.5); 2.508(93.6); 2.504(118.7); 2.499(85.4); 2.335(0.6); 2.330(0.7); 2.326(0.5); 2.131(0.4);
2.119(0.7); 2.100(0.7); 2.086(1.0); 2.072(0.8); 2.054(0.7); 2.027(0.3); 1.802(0.6); 1.797(0.6); 1.778(1.0); 1.765(1.5); 1.754(1.2); 1.740(0.9); 1.715(0.8); 1.694(0.4); 1.577(0.6);
1.567(0.4); 1.557(0.6); 1.546(0.6); 1.526(0.5); 0.000(8.2)
Example 129: ¹H-NMR(400.0 MHz. DMSO): δ = 8.315(1.3); 8.225(4.1); 8.204(4.1); 7.726(15.6); 7.713(16.0); 7.399(2.5); 7.393(2.5); 7.379(2.6); 7.374(2.8); 7.368(2.7); 7.363(2.6);
7.348(4.9); 7.343(2.7); 7.327(5.5); 7.321(2.7); 7.305(3.4); 7.299(5.3); 7.278(3.0); 7.156(2.5); 7.150(2.5); 7.146(2.6); 7.140(2.6); 7.135(2.2); 7.129(1.9); 7.124(1.9); 7.046(15.7);
7.033(15.2); 4.302(0.9); 4.281(3.0); 4.258(3.9); 4.237(3.1); 4.216(0.9); 3.902(4.4); 3.357(1.6); 3.325(741.3); 3.286(0.5); 3.175(0.8); 3.162(0.7); 3.109(1.5); 3.085(3.4); 3.065(3.0);
3.041(1.3); 2.680(1.0); 2.675(2.2); 2.671(3.0); 2.666(2.2); 2.662(1.0); 2.541(1.8); 2.524(7.7); 2.519(13.2); 2.511(180.2); 2.506(361.9); 2.502(471.3); 2.497(332.0); 2.493(154.2);
2.338(1.1); 2.333(2.2); 2.329(3.0); 2.324(2.1); 2.320(1.0); 2.148(0.7); 2.129(1.6); 2.113(3.6); 2.097(4.2); 2.080(4.1); 2.064(2.1); 2.049(1.0); 1.821(1.2); 1.799(4.0); 1.782(5.0);
1.779(4.9); 1.763(4.4); 1.750(3.6); 1.739(1.4); 1.726(2.7); 1.711(1.6); 1.706(1.8); 1.700(2.1); 1.680(1.5); 1.669(1.2); 1.648(2.5); 1.638(1.3); 1.628(2.4); 1.618(2.6); 1.607(1.0);
1.597(2.2); 1.576(0.7); 0.146(0.5); 0.008(4.5); 0.000(130.6); −0.009(4.1); −0.150(0.5)
Example 130: ¹H-NMR(400.0 MHz. DMSO): δ = 8.584(5.4); 8.563(5.4); 8.314(1.7); 7.341(3.5); 7.320(8.1); 7.314(6.5); 7.310(4.2); 7.298(5.9); 7.292(9.5); 7.285(4.0); 7.279(3.8);
7.271(4.4); 7.265(3.5); 7.259(3.4); 7.090(3.4); 7.083(3.5); 7.079(3.6); 7.073(3.7); 7.068(3.1); 7.063(2.9); 7.057(2.7); 4.326(0.9); 4.316(1.7); 4.298(5.4); 4.288(11.8); 4.275(11.4);
4.265(5.3); 4.247(1.7); 4.237(0.9); 4.073(1.3); 4.052(4.0); 4.031(4.8); 4.028(4.9); 4.007(4.1); 3.986(1.3); 3.902(6.3); 3.398(0.3); 3.328(1261.0); 3.247(0.6); 3.195(0.4); 3.185(0.5);
3.161(11.0); 3.150(16.0); 3.139(10.1); 3.116(0.5); 2.984(2.1); 2.959(4.9); 2.938(3.7); 2.915(1.6); 2.695(0.3); 2.680(1.3); 2.676(2.8); 2.671(3.8); 2.666(2.7); 2.662(1.2); 2.541(1.7);
2.524(10.0); 2.511(233.1); 2.507(465.5); 2.502(603.8); 2.497(425.5); 2.493(197.1); 2.376(0.3); 2.338(1.5); 2.333(3.0); 2.329(3.9); 2.324(2.8); 2.320(1.4); 2.061(2.8); 2.049(4.6);
2.043(4.1); 2.029(5.9); 2.013(4.0); 2.002(2.5); 1.995(3.1); 1.976(1.5); 1.778(1.3); 1.770(1.9); 1.752(4.7); 1.735(7.3); 1.729(5.0); 1.715(5.8); 1.708(4.6); 1.692(4.2); 1.681(1.9);
1.674(2.3); 1.667(3.1); 1.647(2.0); 1.642(1.3); 1.623(0.8); 1.509(1.3); 1.488(3.0); 1.477(1.7); 1.467(3.1); 1.456(2.8); 1.447(1.6); 1.436(2.6); 1.415(1.1); 1.236(0.4); 0.008(2.1);
0.000(61.4); −0.009(1.9)
Example 131: ¹H-NMR(400.0 MHz. DMSO): δ = 8.458(2.7); 8.438(2.7); 8.313(16.0); 7.842(4.6); 7.824(4.6); 7.427(2.1); 7.424(2.2); 7.408(6.1); 7.389(4.4); 7.387(4.6); 7.382(2.2);
7.376(3.2); 7.362(1.9); 7.355(4.2); 7.348(1.9); 7.333(2.2); 7.327(3.3); 7.306(1.8); 7.178(1.8); 7.156(7.6); 7.140(4.4); 7.135(7.0); 7.130(3.3); 7.115(2.5); 7.111(1.9); 4.277(0.6);
4.256(2.1); 4.234(2.7); 4.213(2.1); 4.193(0.7); 3.902(1.8); 3.421(0.5); 3.336(1032.1); 3.241(0.6); 3.078(1.1); 3.054(2.5); 3.032(2.3); 3.010(1.0); 2.676(1.6); 2.671(2.1); 2.667(1.5);
2.541(1.3); 2.511(132.5); 2.507(250.8); 2.502(316.7); 2.498(227.0); 2.334(1.6); 2.329(2.0); 2.325(1.5); 2.176(0.5); 2.157(1.1); 2.142(1.7); 2.128(2.0); 2.111(2.3); 2.099(2.0); 2.092(2.0);
2.081(1.7); 2.071(1.3); 2.052(0.7); 1.811(0.9); 1.794(2.6); 1.777(3.7); 1.757(2.8); 1.747(1.9); 1.721(2.0); 1.701(1.5); 1.695(1.6); 1.685(1.1); 1.675(1.3); 1.666(2.0); 1.646(1.8);
1.635(1.7); 1.625(0.8); 1.615(1.4); 1.594(0.5); 1.056(0.4); 0.000(0.7)
Example 132: ¹H-NMR(400.0 MHz. DMSO): δ = 8.314(4.4); 7.895(1.1); 7.873(1.1); 7.377(0.6); 7.372(0.6); 7.357(0.7); 7.352(0.7); 7.346(0.7); 7.341(0.7); 7.336(0.8); 7.326(0.7);
7.321(0.8); 7.315(1.4); 7.309(0.7); 7.293(0.9); 7.287(1.4); 7.266(0.7); 7.129(0.7); 7.123(0.7); 7.118(0.7); 7.113(0.7); 7.108(0.6); 7.102(0.6); 7.097(0.5); 6.838(1.5); 6.833(2.3);
6.828(1.6); 6.720(1.6); 6.715(1.6); 6.710(1.8); 6.706(1.5); 5.972(1.9); 5.965(2.1); 5.962(2.0); 5.956(1.8); 4.263(0.8); 4.241(1.0); 4.218(0.8); 3.902(1.0); 3.735(16.0); 3.332(272.4);
3.175(0.5); 3.162(0.5); 3.122(0.4); 3.097(0.9); 3.076(0.8); 3.052(0.4); 2.676(0.5); 2.671(0.6); 2.667(0.4); 2.542(0.4); 2.525(1.7); 2.511(38.2); 2.507(74.1); 2.502(94.6); 2.498(67.3);
2.493(31.9); 2.334(0.5); 2.329(0.6); 2.325(0.4); 2.092(0.7); 2.078(1.1); 2.063(1.1); 2.058(0.9); 2.045(0.7); 1.809(0.3); 1.788(1.1); 1.772(1.3); 1.769(1.3); 1.752(1.3); 1.738(0.9);
1.728(0.4); 1.716(0.7); 1.701(0.5); 1.690(0.6); 1.669(0.4); 1.599(0.6); 1.589(0.4); 1.578(0.6); 1.568(0.7); 1.547(0.6)
Example 133: ¹H-NMR(400.0 MHz. DMSO): δ = 8.315(1.2); 7.889(1.1); 7.868(1.1); 7.478(3.0); 7.473(3.0); 7.377(0.6); 7.372(0.7); 7.357(0.7); 7.352(0.7); 7.347(0.8); 7.342(1.2);
7.326(0.7); 7.321(1.9); 7.315(0.7); 7.299(0.8); 7.293(1.3); 7.272(0.7); 7.124(0.7); 7.118(0.7); 7.113(0.7); 7.108(0.7); 7.102(0.6); 7.097(0.5); 7.092(0.5); 6.811(2.9); 6.806(2.8);
4.292(0.8); 4.269(1.0); 4.247(0.8); 3.902(0.6); 3.369(0.3); 3.328(186.6); 3.117(0.4); 3.092(0.9); 3.072(0.8); 3.047(0.4); 2.676(0.4); 2.671(0.5); 2.667(0.4); 2.541(0.3); 2.524(1.4);
2.511(34.2); 2.507(67.8); 2.502(88.0); 2.498(62.9); 2.493(30.0); 2.413(16.0); 2.333(0.4); 2.329(0.5); 2.324(0.4); 2.108(0.9); 2.093(1.2); 2.076(1.2); 2.058(0.9); 2.042(0.4); 1.813(0.4);
1.792(1.1); 1.776(1.4); 1.756(1.2); 1.741(0.9); 1.724(0.4); 1.714(0.7); 1.698(0.5); 1.688(0.6); 1.666(0.4); 1.592(0.7); 1.571(0.6); 1.561(0.7); 1.540(0.6); 0.000(1.5)
Example 134: ¹H-NMR(400.0 MHz. DMSO): δ = 8.315(1.8); 7.772(1.4); 7.751(1.4); 7.364(0.8); 7.358(0.8); 7.344(0.9); 7.339(1.6); 7.333(0.9); 7.327(0.9); 7.317(1.9); 7.312(1.5);
7.296(1.1); 7.290(1.6); 7.268(0.9); 7.112(0.9); 7.106(0.9); 7.101(0.9); 7.097(0.9); 7.092(0.7); 7.086(0.7); 7.080(0.7); 6.400(3.6); 4.261(0.9); 4.238(1.2); 4.216(0.9); 3.902(0.7);
3.328(233.1); 3.105(0.5); 3.080(1.1); 3.060(1.0); 3.035(0.5); 2.676(0.6); 2.671(0.7); 2.667(0.5); 2.541(0.5); 2.511(48.8); 2.507(93.7); 2.502(119.8); 2.498(85.7); 2.493(41.0);
2.362(16.0); 2.338(0.4); 2.333(0.6); 2.329(0.8); 2.324(0.6); 2.188(12.5); 2.110(0.4); 2.094(1.0); 2.077(1.5); 2.062(1.5); 2.044(1.1); 2.032(0.5); 2.026(0.5); 1.802(0.4); 1.781(1.3);
1.765(1.7); 1.750(1.2); 1.744(1.5); 1.731(1.2); 1.716(0.5); 1.705(0.9); 1.689(0.6); 1.679(0.7); 1.657(0.5); 1.587(0.4); 1.566(0.8); 1.556(0.4); 1.545(0.8); 1.535(0.9); 1.524(0.4);
1.514(0.8); 0.000(3.5)
Example 135: ¹H-NMR(400.0 MHz. DMSO): δ = 10.813(0.5); 7.899(1.0); 7.791(5.4); 7.429(0.9); 7.408(0.9); 7.379(0.9); 7.374(0.9); 7.360(1.2); 7.354(1.4); 7.349(1.0); 7.343(1.1);
7.335(1.4); 7.330(1.3); 7.324(1.3); 7.314(1.8); 7.309(1.3); 7.292(1.1); 7.287(1.5); 7.266(0.8); 7.100(1.2); 7.091(1.1); 4.283(0.9); 4.261(1.2); 4.238(0.9); 4.137(0.4); 3.903(1.4);
3.615(16.0); 3.328(89.0); 3.029(0.5); 3.004(1.1); 2.984(1.0); 2.960(0.4); 2.897(0.4); 2.877(0.4); 2.676(0.5); 2.671(0.7); 2.541(0.7); 2.507(88.5); 2.502(107.0); 2.498(74.6); 2.333(0.5);
2.329(0.6); 2.136(0.5); 2.121(0.9); 2.102(1.1); 2.085(1.3); 2.073(1.0); 2.064(1.2); 2.054(1.0); 2.044(0.8); 2.033(0.7); 1.805(0.4); 1.785(1.4); 1.766(1.9); 1.756(1.5); 1.748(1.6);
1.734(1.3); 1.726(1.0); 1.713(1.3); 1.703(1.3); 1.685(0.8); 1.675(1.0); 1.654(0.7); 1.629(0.4); 1.576(0.4); 1.555(0.9); 1.545(0.4); 1.535(0.9); 1.524(0.8); 1.514(0.5); 1.503(0.7);
1.494(0.4); 1.482(0.4); 1.473(0.3); 0.000(8.5)
Example 136: ¹H-NMR(400.0 MHz. DMSO): δ = 8.307(1.4); 7.971(1.1); 7.950(1.1); 7.507(2.4); 7.495(2.5); 7.380(0.6); 7.375(0.6); 7.360(0.7); 7.355(0.7); 7.345(1.1); 7.324(1.7);
7.302(0.9); 7.296(1.1); 7.275(0.6); 7.134(0.8); 7.128(0.8); 7.124(0.8); 7.118(0.8); 7.113(0.7); 7.107(0.6); 7.103(0.6); 6.899(3.0); 6.887(2.9); 4.268(0.8); 4.245(1.0); 4.223(0.8);
3.897(0.9); 3.333(169.6); 3.327(171.0); 3.131(0.4); 3.105(1.0); 3.085(0.9); 3.060(0.4); 2.677(0.5); 2.673(0.7); 2.668(0.5); 2.543(0.4); 2.526(1.9); 2.512(43.3); 2.508(85.9); 2.504(110.8);
2.499(78.6); 2.494(36.8); 2.335(0.5); 2.330(0.7); 2.326(0.5); 2.257(16.0); 2.124(0.4); 2.111(0.8); 2.092(1.1); 2.076(1.4); 2.058(1.0); 2.048(0.5); 1.816(0.3); 1.797(1.2); 1.780(1.5);
1.776(1.5); 1.766(1.1); 1.759(1.6); 1.750(1.0); 1.731(0.9); 1.712(0.5); 1.706(0.7); 1.685(0.4); 1.656(0.4); 1.634(0.7); 1.625(0.4); 1.613(0.7); 1.603(0.8); 1.591(0.3); 1.582(0.7);
0.008(0.4); 0.000(11.7); −0.009(0.4)
Example 138: ¹H-NMR(400.0 MHz. DMSO): δ = 8.307(1.9); 7.931(3.9); 7.255(5.0); 7.252(5.2); 7.244(9.3); 7.229(2.3); 7.222(2.5); 7.212(0.9); 7.208(0.9); 7.197(0.4); 7.109(2.9);
6.973(1.6); 4.391(0.6); 4.380(0.9); 4.367(0.8); 4.361(0.7); 4.350(0.8); 4.337(0.6); 3.897(1.8); 3.870(16.0); 3.615(1.3); 3.333(217.0); 3.331(210.9); 3.327(244.1); 3.178(0.6); 3.165(0.6);
2.682(0.4); 2.677(0.7); 2.672(1.0); 2.668(0.7); 2.663(0.3); 2.543(0.6); 2.526(3.0); 2.512(61.5); 2.508(122.6); 2.503(159.1); 2.499(111.7); 2.494(51.2); 2.339(0.4); 2.335(0.7); 2.330(1.0);
2.326(0.7); 2.228(0.6); 2.220(0.8); 2.211(1.1); 2.202(0.8); 2.038(0.7); 2.007(1.5); 1.988(1.0); 1.958(1.0); 1.951(0.9); 1.920(1.8); 1.896(1.8); 1.866(1.0); 1.857(1.4); 1.846(1.2);
1.837(0.6); 1.824(0.5); 1.814(0.4); 1.674(0.7); 1.645(0.8); 1.528(0.6); 1.509(0.5); 1.499(0.5); 0.347(0.7); 0.339(0.6); 0.334(0.8); 0.323(1.0); 0.317(0.6); 0.310(0.8); 0.294(0.4);
0.175(0.8); 0.170(0.5); 0.162(0.9); 0.157(0.9); 0.152(0.8); 0.145(0.7); 0.139(0.6); 0.008(1.5); 0.000(42.1); −0.009(1.2); −0.094(0.5); −0.108(0.8); −0.119(0.8); −0.135(0.5); −0.149(0.3); −1.018
(0.7); −1.030(0.7)
Example 139: ¹H-NMR(400.0 MHz. DMSO): δ = 7.281(7.7); 7.273(5.3); 7.269(5.2); 7.249(1.0); 7.238(1.0); 7.231(1.2); 7.221(0.8); 7.216(0.9); 7.207(0.5); 4.442(0.4); 4.432(0.7);
4.417(0.6); 4.401(0.7); 4.389(0.4); 3.902(1.5); 3.581(12.0); 3.327(101.6); 2.671(0.4); 2.541(0.4); 2.506(48.0); 2.502(57.8); 2.497(39.6); 2.328(0.3); 2.107(16.0); 2.069(0.8); 2.063(0.8);
2.052(0.9); 2.044(1.0); 2.013(1.3); 1.988(1.0); 1.957(1.0); 1.921(1.1); 1.892(1.5); 1.871(1.6); 1.863(1.4); 1.839(0.6); 1.831(0.5); 1.630(0.6); 1.622(0.7); 1.607(0.6); 1.531(0.5);
1.512(0.4); 1.504(0.4); 0.346(0.6); 0.339(0.5); 0.334(0.7); 0.328(0.7); 0.323(0.8); 0.310(0.7); 0.155(0.6); 0.142(0.7); 0.137(0.7); 0.125(0.5); 0.119(0.5); 0.000(7.8); −0.009(0.8); −0.844
(0.6)
Example 140: ¹H-NMR(400.0 MHz. DMSO): δ = 8.315(0.7); 7.305(2.3); 7.287(5.8); 7.269(7.1); 7.240(10.3); 7.234(5.3); 7.222(5.4); 7.214(4.0); 7.200(1.1); 7.197(1.4); 7.193(1.0);
7.181(4.7); 7.179(4.7); 3.902(7.7); 3.683(0.6); 3.510(16.0); 3.389(0.4); 3.371(0.6); 3.352(1.2); 3.324(389.8); 2.675(1.4); 2.671(1.9); 2.666(1.4); 2.662(0.6); 2.541(0.9); 2.524(4.4);
2.511(114.9); 2.506(230.9); 2.502(300.6); 2.497(214.3); 2.493(101.3); 2.337(0.8); 2.333(1.5); 2.328(2.1); 2.324(1.6); 2.319(0.8); 2.220(0.5); 2.069(1.7); 1.860(1.1); 1.824(2.0);
1.785(2.4); 1.745(2.7); 1.717(1.7); 1.576(0.4); 1.537(0.5); 1.521(0.7); 1.505(0.5); 1.490(0.5); 1.454(0.6); 1.421(1.1); 1.388(1.5); 1.371(1.4); 1.350(1.4); 1.319(0.8); 1.259(0.5);
1.249(0.5); 1.241(0.7); 1.230(0.7); 1.223(0.4); 1.212(0.3); 0.374(0.5); 0.358(1.2); 0.342(1.5); 0.321(1.1); 0.146(0.6); 0.091(0.9); 0.008(2.6); 0.000(81.7); −0.009(2.8); −0.150(0.4)
Example 141: ¹H-NMR(400.0 MHz. DMSO): δ = 8.315(0.7); 7.376(0.4); 7.348(2.5); 7.329(5.7); 7.312(6.8); 7.275(5.1); 7.264(8.5); 7.260(6.9); 7.245(5.2); 7.220(1.1); 7.155(0.3);
3.902(9.3); 3.760(0.7); 3.506(0.4); 3.439(0.4); 3.414(0.6); 3.327(165.5); 3.223(0.4); 3.219(0.4); 3.217(0.4); 3.196(0.4); 3.184(0.3); 3.168(0.5); 3.131(0.4); 3.114(0.7); 3.101(0.7);
3.095(0.7); 3.083(0.7); 3.064(0.4); 2.680(0.7); 2.675(1.4); 2.671(1.9); 2.666(1.4); 2.662(0.7); 2.608(16.0); 2.562(0.5); 2.548(0.9); 2.541(0.7); 2.533(0.8); 2.524(3.9); 2.511(100.5);
2.506(205.9); 2.502(272.5); 2.497(196.6); 2.493(94.3); 2.431(0.6); 2.426(0.5); 2.392(0.4); 2.389(0.4); 2.382(0.5); 2.366(0.4); 2.347(0.4); 2.337(1.0); 2.333(1.7); 2.328(2.2); 2.324(1.7);
2.291(0.3); 2.269(0.3); 2.244(0.3); 2.231(0.4); 2.106(0.3); 2.093(0.3); 2.087(0.4); 2.016(1.7); 1.959(0.4); 1.953(0.4); 1.934(0.4); 1.931(0.5); 1.926(0.5); 1.828(4.4); 1.749(2.2);
1.720(2.2); 1.656(0.5); 1.648(0.5); 1.621(0.6); 1.612(0.6); 1.600(0.6); 1.597(0.6); 1.580(0.7); 1.538(0.7); 1.519(0.8); 1.483(0.9); 1.452(1.5); 1.444(1.4); 1.419(1.6); 1.386(1.5);
1.352(1.5); 1.321(1.1); 1.260(0.5); 1.244(0.7); 1.236(0.7); 1.231(0.8); 1.212(0.4); 1.195(1.1); 1.177(2.2); 1.159(1.5); 1.140(0.4); 0.892(0.4); 0.853(0.7); 0.832(0.7); 0.812(0.7);
0.789(0.6); 0.759(0.4); 0.739(0.4); 0.711(0.3); 0.708(0.4); 0.561(0.3); 0.543(0.4); 0.534(0.4); 0.515(0.4); 0.426(0.8); 0.348(1.1); 0.327(1.1); 0.313(1.0); 0.214(0.5); 0.156(0.4);
0.146(0.4); 0.008(0.6); 0.000(16.7); −0.009(0.6)
Example 142: ¹H-NMR(400.0 MHz. DMSO): δ = 8.315(0.6); 7.309(2.2); 7.291(5.1); 7.273(6.1); 7.237(9.4); 7.219(5.9); 7.198(1.3); 3.902(4.0); 3.818(16.0); 3.324(309.6); 2.891(0.7);
2.731(0.6); 2.675(1.0); 2.671(1.4); 2.666(1.0); 2.524(2.7); 2.510(89.6); 2.506(180.9); 2.502(235.9); 2.497(168.4); 2.492(79.7); 2.333(1.4); 2.328(1.8); 2.324(1.3); 2.094(1.5); 1.821(2.1);
1.792(2.6); 1.748(1.8); 1.719(1.3); 1.553(0.4); 1.463(0.5); 1.431(0.9); 1.395(1.2); 1.377(1.3); 1.351(1.0); 1.318(0.7); 0.865(0.3); 0.398(1.1); 0.382(1.4); 0.146(0.4); 0.081(1.1);
0.067(1.1); 0.008(2.4); 0.000(66.9); −0.009(2.1); −0.150(0.3)
Example 143: ¹H-NMR(400.0 MHz. DMSO): δ = 8.315(0.5); 7.482(0.5); 7.281(1.3); 7.262(3.8); 7.244(5.0); 7.215(6.5); 7.210(4.5); 7.196(4.1); 7.190(3.6); 7.173(1.2); 3.902(3.4);
3.794(16.0); 3.323(264.4); 2.675(0.9); 2.670(1.2); 2.666(0.9); 2.541(0.3); 2.524(2.4); 2.510(75.3); 2.506(152.8); 2.501(201.2); 2.497(144.6); 2.492(69.4); 2.333(1.1); 2.328(1.5);
2.323(1.1); 2.128(1.2); 1.815(2.2); 1.787(2.0); 1.742(1.3); 1.715(1.1); 1.426(0.8); 1.395(1.0); 1.372(1.1); 1.316(0.6); 0.518(1.0); 0.503(1.3); 0.168(0.8); 0.146(0.8); 0.008(2.1);
0.000(50.1); −0.009(1.4)
Example 144: ¹H-NMR(400.0 MHz. DMSO): δ = 15.728(0.4); 7.834(0.4); 7.525(0.3); 7.285(1.5); 7.266(4.0); 7.248(4.3); 7.220(4.4); 7.203(3.6); 7.182(2.2); 7.165(0.9); 6.837(0.4);
5.364(0.4); 5.324(0.4); 3.545(0.4); 3.504(11.3); 3.465(0.5); 3.453(0.5); 3.447(0.6); 3.423(0.6); 3.409(0.7); 3.399(0.7); 3.384(0.8); 3.321(1261.7); 2.675(11.2); 2.670(15.3); 2.666(11.6);
2.661(5.9); 2.609(0.5); 2.582(0.9); 2.540(13.2); 2.510(971.5); 2.506(1890.0); 2.501(2453.2); 2.497(1805.8); 2.493(909.7); 2.406(1.2); 2.374(1.0); 2.332(11.7); 2.328(16.0); 2.323(12.1);
2.273(1.7); 2.245(1.1); 2.196(0.4); 2.183(0.4); 2.150(0.6); 2.124(1.4); 2.096(0.5); 2.076(0.4); 2.056(0.4); 2.027(0.5); 2.008(0.6); 1.989(0.7); 1.851(1.0); 1.838(1.0); 1.810(1.6);
1.780(1.9); 1.737(1.4); 1.729(1.2); 1.718(1.2); 1.632(0.4); 1.573(0.6); 1.553(0.4); 1.547(0.5); 1.530(0.4); 1.524(0.4); 1.513(0.5); 1.501(0.4); 1.492(0.6); 1.453(0.7); 1.449(0.7);
1.432(1.0); 1.417(0.8); 1.394(1.0); 1.386(0.8); 1.374(1.1); 1.353(0.8); 1.343(0.8); 1.323(0.7); 1.290(0.7); 1.277(0.7); 1.266(0.9); 1.253(1.3); 1.236(2.9); 0.957(3.1); 0.869(0.5);
0.854(0.8); 0.837(0.6); 0.775(0.3); 0.428(0.9); 0.198(0.4); 0.168(0.4); 0.146(2.2); 0.126(0.4); 0.008(21.6); 0.000(462.5); −0.009(21.3); −0.088(11.2); −0.150(2.9); −0.182(0.6); −0.200(0.6);
−0.215(0.6); −0.222(0.5); −0.237(0.4); −0.263(0.4); −3.078(0.3); −3.190(0.3); −3.331(0.4); −3.433(0.4); −3.513(0.3); −3.616(0.4); −3.649(0.4)
Example 145: ¹H-NMR(400.0 MHz. DMSO): δ = 8.315(0.4); 7.302(1.5); 7.284(4.1); 7.266(4.7); 7.243(4.4); 7.226(2.4); 7.217(2.0); 7.213(2.0); 7.196(2.4); 7.179(1.1); 7.154(0.5);
3.902(5.1); 3.868(0.4); 3.742(16.0); 3.325(247.3); 3.175(0.4); 3.162(0.3); 2.891(0.7); 2.732(0.6); 2.675(0.8); 2.671(1.1); 2.666(0.8); 2.541(0.6); 2.524(2.5); 2.511(68.6); 2.506(137.2);
2.502(177.3); 2.497(126.3); 2.493(59.7); 2.333(1.0); 2.328(1.3); 2.324(1.0); 2.220(0.4); 2.129(1.0); 1.823(2.4); 1.798(2.0); 1.747(1.2); 1.719(1.1); 1.521(0.5); 1.427(0.8); 1.394(1.0);
1.375(1.0); 1.354(0.9); 1.323(0.6); 1.297(0.4); 1.286(0.4); 1.269(0.4); 1.251(0.4); 0.370(1.0); 0.050(0.9); 0.039(0.8); 0.008(0.5); 0.000(9.2)
Example 146: ¹H-NMR(400.0 MHz. DMSO): δ = 8.315(0.6); 7.308(1.4); 7.288(6.5); 7.272(16.0); 7.254(2.3); 7.231(0.6); 7.219(0.9); 7.210(1.9); 7.205(2.0); 7.189(2.7); 7.180(1.3);
7.173(1.3); 3.902(6.3); 3.657(3.5); 3.636(0.6); 3.521(3.7); 3.348(0.4); 3.322(225.0); 3.285(0.5); 3.267(0.5); 3.253(0.4); 3.236(0.4); 3.175(0.5); 3.163(0.6); 2.891(0.9); 2.732(0.7);
2.675(0.9); 2.670(1.3); 2.666(1.0); 2.541(0.4); 2.524(2.1); 2.510(88.0); 2.506(179.1); 2.502(234.3); 2.497(168.9); 2.493(82.1); 2.464(1.4); 2.419(4.4); 2.393(0.5); 2.359(0.4); 2.333(1.3);
2.328(1.7); 2.324(1.4); 2.306(0.5); 2.280(0.6); 2.268(0.5); 2.248(4.3); 2.220(0.6); 2.195(0.5); 2.153(1.4); 2.126(1.2); 2.087(0.8); 2.069(0.8); 2.046(1.0); 2.030(0.8); 2.016(0.8);
1.952(0.9); 1.940(0.9); 1.923(1.5); 1.915(1.4); 1.904(0.8); 1.846(2.1); 1.808(3.9); 1.783(3.5); 1.740(2.3); 1.711(2.0); 1.666(0.6); 1.537(0.5); 1.521(0.7); 1.442(1.8); 1.409(1.8);
1.381(1.9); 1.351(1.6); 1.319(1.2); 1.287(0.7); 1.260(0.5); 1.250(0.6); 1.235(0.7); 1.202(0.5); 1.150(0.7); 1.132(0.7); 1.113(0.7); 1.060(0.6); 0.994(0.3); 0.707(0.5); 0.685(0.5);
0.677(0.5); 0.638(0.4); 0.627(0.4); 0.619(0.4); 0.585(0.3); 0.294(0.9); 0.215(0.4); 0.008(0.6); 0.000(12.3); −0.008(0.7); −0.033(0.3)
Example 147: ¹H-NMR(400.0 MHz. DMSO): δ = 8.305(5.2); 7.480(0.4); 7.349(2.1); 7.332(6.1); 7.313(5.4); 7.274(2.9); 7.256(4.2); 7.248(9.1); 7.245(9.2); 7.227(6.8); 3.896(7.1);
3.335(585.0); 3.330(813.9); 2.730(1.3); 2.677(1.9); 2.673(2.5); 2.668(1.9); 2.629(16.0); 2.543(1.2); 2.526(6.1); 2.512(142.1); 2.508(278.2); 2.504(359.4); 2.499(259.4); 2.495(126.3);
2.335(1.9); 2.331(2.5); 2.326(1.9); 1.929(1.8); 1.831(2.7); 1.800(2.3); 1.754(3.0); 1.456(1.2); 1.424(1.6); 1.408(1.2); 1.389(1.3); 1.349(1.2); 1.319(0.9); 1.261(0.3); 1.238(0.4);
0.812(0.4); 0.387(1.3); 0.228(1.3); 0.008(1.0); 0.000(19.4); −0.008(0.8)
Example 148: ¹H-NMR(400.0 MHz. DMSO): δ = 10.696(3.0); 10.683(2.9); 8.315(1.0); 7.740(4.9); 7.720(6.3); 7.698(2.2); 7.680(5.4); 7.661(3.7); 7.597(3.6); 7.578(4.8); 7.559(1.9);
7.481(0.4); 7.473(0.4); 7.459(0.4); 7.427(5.7); 7.408(5.0); 7.315(4.7); 7.311(1.9); 7.296(9.8); 7.283(3.3); 7.278(10.1); 7.268(0.9); 7.257(1.0); 7.240(0.5); 7.219(16.0); 7.200(11.4);
7.181(1.5); 7.178(2.2); 7.174(1.1); 3.902(3.8); 3.600(1.2); 3.587(2.5); 3.578(2.9); 3.574(2.6); 3.568(2.9); 3.560(2.6); 3.556(1.6); 3.546(1.2); 3.326(335.4); 3.324(320.7); 2.680(0.9);
2.675(1.7); 2.670(2.4); 2.666(1.7); 2.661(0.8); 2.563(0.5); 2.549(0.8); 2.541(1.7); 2.524(6.2); 2.519(10.0); 2.510(136.2); 2.506(276.6); 2.501(362.8); 2.497(257.0); 2.492(119.3);
2.337(0.8); 2.333(1.7); 2.328(2.3); 2.324(1.6); 2.319(0.7); 2.191(1.5); 1.439(0.8); 1.425(2.5); 1.412(5.4); 1.394(6.5); 1.377(4.0); 1.362(1.1); 1.236(0.3); 0.146(0.5); 0.008(3.8);
0.000(117.8); −0.009(3.6); −0.150(0.5)
Example 149: ¹H-NMR(400.0 MHz. DMSO): δ = 10.446(3.3); 10.426(3.3); 8.315(1.4); 7.678(9.5); 7.665(9.8); 7.640(0.5); 7.636(0.4); 7.581(0.4); 7.573(0.4); 7.562(0.4); 7.554(0.4);
7.342(3.1); 7.321(6.5); 7.315(3.4); 7.299(4.0); 7.294(6.4); 7.272(3.5); 7.256(3.1); 7.250(3.2); 7.236(3.2); 7.231(3.5); 7.225(3.4); 7.220(3.2); 7.205(3.0); 7.200(3.1); 7.184(16.0);
7.171(15.1); 7.079(3.3); 7.074(3.4); 7.069(3.5); 7.064(3.5); 7.059(3.0); 7.053(2.8); 7.048(2.7); 5.160(1.3); 5.142(3.4); 5.124(4.3); 5.106(3.4); 5.087(1.3); 3.902(5.1); 3.590(1.9);
3.571(4.5); 3.548(4.4); 3.528(2.1); 3.372(0.5); 3.323(582.2); 2.675(2.9); 2.671(4.0); 2.666(2.9); 2.662(1.3); 2.541(2.2); 2.524(10.2); 2.510(247.7); 2.506(487.2); 2.502(628.1);
2.497(445.2); 2.493(209.5); 2.412(0.3); 2.333(3.1); 2.328(4.1); 2.324(3.0); 2.168(1.2); 2.159(1.4); 2.146(1.9); 2.137(2.9); 2.128(3.0); 2.119(2.8); 2.113(2.4); 2.109(2.5); 2.095(2.3);
2.088(2.2); 2.075(1.6); 2.065(2.6); 2.056(2.5); 2.045(3.9); 2.038(3.8); 2.025(3.4); 2.005(3.7); 1.982(4.3); 1.975(1.9); 1.959(2.9); 1.951(2.7); 1.936(2.4); 1.930(2.6); 1.923(2.9);
1.915(2.7); 1.904(2.6); 1.895(3.0); 1.886(1.9); 1.874(1.5); 1.866(0.9); 1.817(1.8); 1.802(2.0); 1.796(2.7); 1.781(3.4); 1.776(2.1); 1.769(1.8); 1.763(2.9); 1.748(2.3); 1.729(1.4);
1.718(0.6); 1.707(0.7); 1.696(0.6); 1.688(2.7); 1.680(1.5); 1.667(4.0); 1.659(2.6); 1.645(2.6); 1.637(3.0); 1.624(0.9); 1.616(1.9); 1.594(0.5); 1.244(0.4); 1.235(0.6); 0.146(0.8);
0.008(6.6); 0.000(184.0); −0.008(6.2); −0.150(0.8)
Example 150: ¹H-NMR(400.0 MHz. DMSO): δ = 20.010(0.5); 10.648(0.9); 10.290(2.9); 10.272(3.2); 8.368(1.1); 8.363(1.3); 8.355(1.3); 8.351(1.4); 8.310(3.1); 7.690(0.9); 7.673(1.4);
7.669(1.2); 7.656(1.1); 7.646(1.5); 7.641(2.4); 7.622(1.9); 7.605(0.8); 7.579(1.1); 7.572(1.2); 7.560(1.5); 7.540(2.4); 7.389(1.2); 7.368(2.7); 7.341(1.5); 7.320(0.7); 7.298(1.3);
7.288(2.5); 7.278(1.4); 7.267(5.9); 7.240(4.7); 7.218(2.5); 7.193(2.8); 7.173(3.3); 7.168(3.2); 7.147(2.9); 7.143(2.9); 7.006(3.7); 5.025(1.1); 5.008(2.9); 4.991(3.6); 4.975(3.0);
4.956(1.3); 4.269(0.7); 4.249(7.8); 4.238(16.0); 4.227(8.4); 4.207(0.9); 3.899(3.7); 3.665(0.7); 3.649(1.6); 3.635(1.5); 3.618(0.7); 3.493(2.1); 3.474(4.4); 3.450(4.6); 3.430(2.2);
3.404(0.9); 3.337(654.0); 3.334(745.3); 3.332(751.0); 3.170(0.7); 3.154(1.1); 3.143(1.7); 3.120(5.6); 3.110(12.2); 3.098(11.8); 3.088(5.0); 3.064(1.4); 3.054(1.0); 2.892(0.7); 2.768(1.4);
2.749(1.8); 2.731(1.5); 2.677(2.7); 2.672(3.5); 2.668(2.6); 2.542(3.8); 2.525(10.6); 2.512(224.4); 2.508(431.4); 2.503(548.4); 2.499(389.5); 2.494(185.7); 2.334(2.5); 2.330(3.3);
2.325(2.3); 2.089(2.0); 2.071(3.5); 2.058(4.8); 2.050(5.6); 2.040(8.4); 2.033(6.2); 2.017(6.7); 1.993(3.8); 1.972(2.0); 1.939(1.7); 1.912(3.1); 1.902(3.4); 1.892(3.9); 1.882(2.8);
1.873(2.9); 1.863(2.0); 1.853(1.4); 1.842(1.0); 1.728(1.7); 1.708(3.5); 1.694(3.9); 1.689(4.3); 1.676(5.4); 1.668(5.2); 1.658(4.7); 1.652(4.7); 1.637(2.2); 1.630(3.0); 1.609(1.5);
1.588(0.4); 1.317(0.4); 1.300(0.5); 1.235(0.6); 0.000(16.5)
Example 152: ¹H-NMR(400.0 MHz. DMSO): δ = 8.315(1.0); 7.355(4.0); 7.335(9.4); 7.318(10.3); 7.278(16.0); 7.260(10.9); 7.242(2.0); 7.228(0.3); 7.144(5.7); 5.552(1.2); 5.523(2.1);
5.501(1.3); 5.443(1.4); 3.902(9.3); 3.510(0.4); 3.446(22.6); 3.396(0.3); 3.387(0.3); 3.323(373.0); 3.295(2.7); 3.277(1.6); 3.174(1.4); 3.162(1.3); 2.675(1.8); 2.670(2.5); 2.666(1.8);
2.661(0.9); 2.541(1.6); 2.524(7.0); 2.511(162.7); 2.506(324.3); 2.502(421.9); 2.497(299.0); 2.492(140.5); 2.450(0.7); 2.418(0.4); 2.391(0.3); 2.337(1.1); 2.333(2.0); 2.328(2.7);
2.324(2.0); 2.153(2.7); 2.144(2.0); 2.109(1.9); 2.080(2.1); 1.940(2.0); 1.922(1.9); 1.811(2.3); 1.806(2.3); 1.795(2.2); 1.791(2.2); 1.740(2.9); 1.714(1.1); 1.681(1.7); 1.652(1.6);
1.623(0.8); 1.420(4.5); 1.403(7.1); 1.343(0.7); 1.298(0.4); 1.258(0.3); 1.244(0.3); 1.234(0.5); 1.002(1.4); 0.989(1.8); 0.976(1.9); 0.964(1.5); 0.852(1.6); 0.833(2.0); 0.816(1.5);
0.277(1.5); 0.260(1.9); 0.238(1.4); 0.136(1.8); 0.126(1.7); 0.008(1.7); 0.000(48.6); −0.009(1.5)
Example 153: ¹H-NMR(400.0 MHz. DMSO): δ = 8.315(1.1); 7.364(2.8); 7.314(3.5); 7.297(7.8); 7.278(11.3); 7.264(9.4); 5.526(0.5); 5.511(0.6); 4.979(1.3); 4.395(1.7); 4.029(0.8);
3.902(11.6); 3.800(0.9); 3.618(0.7); 3.591(16.0); 3.569(6.7); 3.384(0.4); 3.324(495.8); 3.268(0.5); 3.259(0.6); 2.675(2.0); 2.671(2.8); 2.666(2.0); 2.541(1.7); 2.524(7.8); 2.510(186.9);
2.506(374.0); 2.502(489.9); 2.497(355.9); 2.493(174.9); 2.412(0.7); 2.372(1.1); 2.333(2.5); 2.328(3.3); 2.324(2.5); 2.280(0.9); 2.247(1.0); 2.215(1.3); 2.174(14.2); 2.086(6.2);
2.029(3.0); 1.985(7.1); 1.973(6.3); 1.915(2.9); 1.869(3.9); 1.787(0.4); 1.710(1.6); 1.537(1.8); 1.520(1.6); 1.235(0.7); 0.835(0.4); 0.388(1.3); 0.380(1.3); 0.331(0.4); 0.326(0.3);
0.257(1.4); 0.108(1.4); 0.008(1.5); 0.000(39.1); −0.008(1.5); −0.340(1.2); −0.594(0.7); −0.620(0.5); −1.272(1.2)
Example 154: ¹H-NMR(400.0 MHz. DMSO): δ = 8.318(0.3); 7.440(1.1); 7.421(1.3); 7.292(0.5); 7.274(1.0); 7.258(1.0); 7.237(1.3); 7.218(1.4); 7.202(0.5); 7.195(0.7); 3.902(3.9);
3.630(0.4); 3.495(7.8); 3.355(0.5); 3.324(129.0); 3.118(1.0); 3.106(1.2); 3.100(3.0); 3.088(3.3); 3.082(3.1); 3.070(3.2); 3.064(1.2); 3.052(1.0); 2.675(0.6); 2.671(0.8); 2.666(0.6);
2.541(0.8); 2.524(2.7); 2.511(53.1); 2.506(104.0); 2.502(134.1); 2.497(94.7); 2.493(44.3); 2.333(0.7); 2.329(0.9); 2.324(0.6); 1.984(0.4); 1.976(0.4); 1.875(7.4); 1.834(1.3); 1.797(1.0);
1.755(0.8); 1.730(0.9); 1.457(0.4); 1.428(0.5); 1.409(0.6); 1.381(0.6); 1.348(0.9); 1.319(0.9); 1.289(0.4); 1.231(0.3); 1.201(7.7); 1.183(16.0); 1.165(7.4); 0.696(0.5); 0.468(0.5);
0.449(0.4); 0.000(6.4)
Example 155: ¹H-NMR(400.0 MHz. DMSO): δ = 8.088(3.7); 7.563(4.5); 7.558(4.7); 7.465(1.7); 7.444(3.8); 7.417(3.0); 7.412(2.6); 7.396(1.3); 7.391(1.2); 7.182(1.3); 7.046(2.9);
6.910(1.4); 4.403(0.5); 4.381(1.2); 4.355(1.5); 4.333(0.7); 4.177(0.6); 4.157(0.8); 4.151(1.2); 4.131(1.2); 4.104(0.5); 3.943(0.7); 3.902(3.0); 3.857(16.0); 3.322(113.5); 2.891(0.4);
2.732(0.3); 2.675(0.7); 2.671(0.9); 2.667(0.7); 2.548(0.6); 2.511(58.7); 2.506(112.5); 2.502(143.9); 2.497(103.1); 2.493(50.0); 2.333(0.6); 2.329(0.8); 2.324(0.6); 2.159(0.4); 2.150(0.6);
2.141(0.9); 2.130(0.7); 2.121(0.9); 2.111(0.8); 2.099(0.6); 2.091(0.8); 2.079(0.6); 2.067(0.9); 2.060(0.9); 2.047(1.0); 2.039(0.6); 2.025(0.7); 2.002(0.7); 1.985(0.8); 1.980(0.7);
1.971(0.5); 1.962(0.6); 1.956(0.5); 1.934(0.5); 1.922(0.4); 1.910(0.6); 1.898(0.6); 1.891(0.8); 1.877(0.7); 1.870(0.6); 1.857(0.4); 1.835(0.4); 1.816(0.6); 1.807(0.7); 1.796(0.7);
1.786(0.7); 1.770(0.5); 1.615(0.4); 1.593(0.8); 1.566(0.9); 1.562(0.8); 1.536(0.6); 0.700(0.7); 0.686(2.5); 0.670(2.5); 0.658(0.8); 0.648(0.5); 0.631(0.4); 0.379(0.5); 0.368(0.5);
0.350(1.1); 0.343(1.2); 0.317(1.1); 0.308(1.3); 0.289(0.6); 0.277(0.6); 0.000(6.8)
Example 156: ¹H-NMR(400.0 MHz. DMSO): δ = 8.315(0.4); 7.489(3.7); 7.408(2.3); 7.403(2.2); 7.397(1.9); 7.390(3.3); 7.385(3.0); 7.380(1.8); 7.373(1.9); 7.219(0.7); 7.213(0.9);
7.200(2.3); 7.195(2.2); 7.190(2.2); 7.184(4.0); 7.176(1.9); 7.172(1.9); 7.167(1.6); 7.153(0.5); 7.149(0.3); 7.049(1.5); 6.913(3.4); 6.777(1.7); 4.531(0.5); 4.510(1.3); 4.488(1.3);
4.467(0.6); 3.902(2.9); 3.897(0.8); 3.877(0.6); 3.792(16.0); 3.693(0.6); 3.675(0.8); 3.668(0.9); 3.652(0.9); 3.643(0.7); 3.625(0.6); 3.322(128.3); 2.675(0.7); 2.671(0.9); 2.666(0.7);
2.661(0.4); 2.541(0.7); 2.524(3.2); 2.511(57.5); 2.506(111.1); 2.502(141.7); 2.497(99.9); 2.493(46.5); 2.470(1.1); 2.452(1.1); 2.442(1.0); 2.434(0.9); 2.424(1.3); 2.382(0.6); 2.373(0.5);
2.352(0.8); 2.345(0.6); 2.333(1.1); 2.328(1.3); 2.324(1.2); 2.106(0.4); 2.085(0.9); 2.062(0.7); 2.053(1.2); 2.033(1.1); 2.002(0.8); 1.981(0.4); 1.911(0.4); 1.894(0.9); 1.879(0.7);
1.865(0.8); 1.846(0.4); 1.629(0.4); 1.607(0.7); 1.580(0.6); 1.557(0.3); 0.583(0.6); 0.565(2.2); 0.556(1.5); 0.547(2.1); 0.528(0.7); 0.520(0.4); 0.504(0.3); 0.224(0.7); 0.213(0.8);
0.199(0.7); 0.137(0.5); 0.127(0.9); 0.112(0.9); 0.102(0.8); 0.095(0.7); 0.000(6.5)
Example 157: ¹H-NMR(400.0 MHz. DMSO): δ = 8.315(0.5); 7.747(2.5); 7.605(1.4); 7.366(1.1); 7.346(1.3); 7.210(0.4); 7.198(0.4); 7.191(0.3); 7.109(1.2); 6.973(2.5); 6.837(1.3);
3.908(0.9); 3.902(4.3); 3.811(16.0); 3.633(0.4); 3.609(0.3); 3.352(0.4); 3.322(165.0); 3.303(0.5); 2.675(0.9); 2.671(1.3); 2.666(0.9); 2.541(1.0); 2.524(3.6); 2.511(78.8); 2.506(156.0);
2.502(202.5); 2.497(144.4); 2.492(68.6); 2.333(0.9); 2.328(1.2); 2.324(0.9); 2.017(0.5); 1.883(1.2); 1.868(1.2); 1.813(1.1); 1.784(1.3); 1.747(1.3); 1.721(1.3); 1.444(0.7); 1.412(1.1);
1.379(1.0); 1.345(0.7); 1.313(0.7); 1.283(0.6); 1.259(0.6); 1.249(0.6); 1.235(0.5); 0.828(0.4); 0.813(0.5); 0.745(0.8); 0.595(0.8); 0.147(0.3); 0.131(0.3); 0.008(0.4); 0.000(10.5); −0.008
(0.4)
Example 158: ¹H-NMR(400.0 MHz. DMSO): δ = 7.589(0.8); 7.455(0.6); 7.440(0.6); 7.244(1.4); 7.225(1.1); 7.203(0.4); 7.197(0.3); 7.052(0.5); 6.917(1.0); 6.781(0.5); 3.908(0.8);
3.902(5.1); 3.792(6.8); 3.323(86.1); 3.174(0.6); 3.162(0.6); 3.118(0.9); 3.106(1.2); 3.100(2.6); 3.088(3.1); 3.082(2.8); 3.070(2.9); 3.064(1.1); 3.052(0.9); 2.891(0.3); 2.675(0.5);
2.671(0.6); 2.666(0.4); 2.541(0.5); 2.524(2.0); 2.511(41.4); 2.506(80.9); 2.502(103.4); 2.497(73.0); 2.493(34.0); 2.333(0.5); 2.329(0.6); 2.324(0.5); 1.883(0.7); 1.825(0.5); 1.811(0.5);
1.795(0.7); 1.755(0.6); 1.728(0.6); 1.452(0.3); 1.419(0.5); 1.387(0.6); 1.355(0.4); 1.323(0.4); 1.290(0.3); 1.201(7.8); 1.183(16.0); 1.165(7.4); 0.751(0.4); 0.571(0.4); 0.000(5.1)
Example 159: ¹H-NMR(400.0 MHz. DMSO): δ = 7.570(3.6); 7.564(3.7); 7.529(0.3); 7.458(1.4); 7.437(3.5); 7.414(2.5); 7.409(2.3); 7.393(1.1); 7.388(1.0); 4.466(0.5); 4.445(1.0);
4.418(1.1); 4.396(0.5); 4.093(0.5); 4.073(0.7); 4.067(0.9); 4.047(0.9); 4.020(0.4); 3.902(2.2); 3.670(0.4); 3.572(11.4); 3.322(107.7); 2.675(0.5); 2.671(0.7); 2.666(0.5); 2.524(2.1);
2.511(48.3); 2.506(93.7); 2.502(120.0); 2.497(85.5); 2.493(41.3); 2.393(0.7); 2.382(0.9); 2.376(0.9); 2.366(0.7); 2.333(0.6); 2.329(0.8); 2.324(0.6); 2.173(0.3); 2.163(0.4); 2.153(0.6);
2.143(0.9); 2.133(0.7); 2.123(0.9); 2.112(0.7); 2.104(0.4); 2.092(0.5); 2.079(0.4); 2.070(0.4); 2.060(0.6); 2.039(1.1); 2.025(1.6); 2.003(1.6); 1.989(16.0); 1.901(0.4); 1.888(0.5);
1.869(0.8); 1.849(0.9); 1.832(0.9); 1.821(0.8); 1.811(0.8); 1.801(0.7); 1.792(0.5); 1.781(0.5); 1.606(0.4); 1.584(0.7); 1.558(0.8); 1.528(0.6); 0.661(0.4); 0.654(0.6); 0.642(0.7);
0.637(0.9); 0.627(0.8); 0.620(1.0); 0.610(1.0); 0.593(0.9); 0.577(0.6); 0.570(0.4); 0.424(0.5); 0.410(0.7); 0.398(0.8); 0.385(0.7); 0.302(0.3); 0.292(0.7); 0.278(0.9); 0.267(0.8);
0.251(0.5); 0.000(7.6)
Example 160: ¹H-NMR(400.0 MHz. DMSO): δ = 8.315(0.4); 7.570(5.4); 7.565(5.4); 7.475(2.5); 7.454(4.6); 7.414(3.3); 7.409(2.9); 7.393(1.7); 7.388(1.5); 4.494(0.6); 4.472(1.5);
4.446(1.6); 4.424(0.7); 4.100(0.7); 4.080(0.9); 4.073(1.4); 4.053(1.4); 4.026(0.6); 3.902(2.3); 3.585(16.0); 3.322(126.6); 2.675(0.8); 2.671(1.0); 2.667(0.8); 2.541(0.8); 2.524(3.6);
2.511(67.0); 2.506(128.6); 2.502(163.9); 2.497(116.8); 2.493(55.8); 2.455(0.4); 2.437(1.1); 2.425(1.2); 2.418(2.9); 2.406(3.4); 2.399(3.6); 2.388(3.5); 2.381(1.9); 2.369(1.4); 2.351(0.4);
2.333(0.8); 2.329(1.0); 2.324(0.7); 2.175(0.4); 2.166(0.5); 2.156(0.8); 2.146(1.1); 2.136(0.9); 2.126(1.1); 2.116(0.9); 2.107(0.5); 2.097(0.5); 2.070(0.5); 2.059(0.7); 2.047(0.9);
2.039(1.2); 2.026(1.3); 2.008(1.3); 1.990(1.2); 1.968(0.7); 1.960(0.5); 1.938(0.4); 1.903(0.4); 1.891(0.6); 1.871(1.0); 1.852(1.1); 1.833(1.1); 1.822(1.0); 1.813(1.0); 1.804(0.9);
1.793(0.6); 1.782(0.5); 1.606(0.5); 1.584(0.9); 1.558(1.1); 1.527(0.8); 1.505(0.3); 1.011(6.9); 0.992(14.7); 0.974(6.5); 0.658(0.5); 0.651(0.8); 0.640(1.0); 0.634(1.3); 0.624(1.6);
0.610(1.6); 0.599(1.3); 0.583(0.8); 0.576(0.6); 0.419(0.4); 0.409(0.7); 0.393(0.8); 0.382(1.1); 0.372(1.0); 0.297(0.5); 0.286(1.0); 0.275(1.2); 0.264(1.0); 0.248(0.7); 0.000(8.2)
Example 161: ¹H-NMR(400.0 MHz. DMSO): δ = 8.315(0.5); 7.595(2.2); 7.390(1.4); 7.370(1.9); 7.266(0.5); 3.902(2.8); 3.644(0.8); 3.528(16.0); 3.321(184.1); 2.671(1.2); 2.666(0.9);
2.510(81.2); 2.506(163.5); 2.502(214.8); 2.497(158.5); 2.493(80.8); 2.329(3.7); 2.311(2.6); 2.293(1.3); 1.980(0.7); 1.822(2.2); 1.790(1.7); 1.754(1.3); 1.728(1.6); 1.426(0.8); 1.402(0.8);
1.372(1.0); 1.344(1.5); 1.323(1.4); 1.297(0.7); 1.235(0.4); 1.150(0.5); 1.132(0.8); 1.113(0.4); 0.987(5.6); 0.968(11.6); 0.950(5.4); 0.697(0.8); 0.497(0.9); 0.099(0.4); 0.000(9.5)
Example 162: ¹H-NMR(400.0 MHz. DMSO): δ = 8.315(0.6); 7.440(1.9); 7.421(2.4); 7.274(1.8); 7.258(2.0); 7.240(2.6); 7.220(2.7); 7.198(1.4); 3.902(2.9); 3.643(0.8); 3.504(16.0);
3.322(175.8); 3.175(1.1); 3.162(1.0); 2.675(0.8); 2.671(1.1); 2.666(0.9); 2.524(1.9); 2.511(79.9); 2.506(164.1); 2.502(217.0); 2.497(156.7); 2.493(76.0); 2.356(1.3); 2.337(3.4);
2.328(2.8); 2.319(3.5); 2.301(1.4); 1.984(0.9); 1.901(1.1); 1.873(1.3); 1.826(2.3); 1.797(1.9); 1.758(1.6); 1.731(1.7); 1.460(0.8); 1.407(1.0); 1.383(1.1); 1.351(1.4); 1.316(1.3);
1.285(0.9); 1.249(0.5); 1.231(0.5); 1.150(0.4); 1.131(0.7); 1.113(0.4); 0.986(6.2); 0.967(12.9); 0.948(5.9); 0.684(0.9); 0.475(0.9); 0.082(0.4); 0.008(0.6); 0.000(11.9); −0.009(0.4)
Example 163: ¹H-NMR(400.0 MHz. DMSO): δ = 8.315(1.3); 7.637(0.5); 7.610(2.0); 7.575(0.8); 7.552(0.6); 7.534(0.9); 7.490(0.4); 7.458(0.4); 7.398(1.6); 7.383(1.9); 7.346(1.5);
7.328(0.8); 7.273(0.5); 7.265(0.5); 7.246(0.6); 7.226(0.5); 7.161(0.3); 6.992(0.3); 5.604(0.5); 3.902(16.0); 3.643(0.4); 3.603(0.4); 3.597(0.4); 3.580(0.4); 3.322(348.2); 3.280(1.3);
3.240(0.5); 3.208(0.3); 3.178(0.3); 2.675(1.6); 2.671(2.2); 2.666(1.5); 2.524(2.8); 2.511(160.6); 2.506(333.0); 2.502(442.7); 2.497(323.1); 2.493(160.0); 2.455(3.8); 2.444(2.2);
2.394(1.3); 2.357(1.1); 2.337(1.7); 2.333(2.8); 2.328(3.6); 2.324(3.0); 2.319(2.4); 2.259(1.2); 2.228(1.7); 2.211(1.8); 2.144(2.5); 2.125(7.5); 2.065(1.5); 2.044(1.2); 2.035(1.1);
2.010(1.2); 1.994(1.2); 1.940(3.2); 1.908(1.9); 1.828(3.2); 1.790(2.8); 1.768(2.1); 1.723(2.9); 1.652(1.0); 1.581(0.8); 1.573(1.0); 1.554(0.8); 1.509(1.0); 1.391(2.0); 1.365(2.2);
1.334(2.4); 1.309(2.1); 1.282(1.4); 1.258(1.3); 1.237(1.4); 1.220(0.9); 1.195(1.0); 1.176(1.1); 1.158(0.8); 1.110(0.4); 1.098(0.4); 1.093(0.4); 1.032(0.4); 0.984(0.3); 0.954(0.4);
0.929(0.4); 0.912(0.5); 0.898(0.5); 0.874(0.6); 0.856(0.8); 0.831(0.8); 0.811(0.8); 0.765(0.8); 0.745(1.0); 0.733(1.1); 0.702(1.3); 0.695(1.4); 0.685(1.4); 0.648(1.1); 0.615(1.0);
0.607(1.0); 0.591(1.0); 0.567(0.9); 0.526(0.8); 0.500(1.0); 0.485(1.0); 0.475(0.9); 0.424(0.6); 0.408(0.6); 0.403(0.6); 0.377(0.7); 0.300(0.4); 0.264(0.3); 0.243(0.4); 0.207(0.4);
0.199(0.4); 0.194(0.4); 0.146(0.4); 0.008(0.7); 0.000(21.3); −0.009(0.9)
Example 164: ¹H-NMR(400.0 MHz. DMSO): δ = 7.409(1.6); 7.401(2.2); 7.395(1.7); 7.386(2.8); 7.376(1.5); 7.226(0.6); 7.216(3.5); 7.207(2.7); 7.201(2.6); 7.193(2.8); 7.183(0.3);
4.573(0.3); 4.551(0.8); 4.530(0.8); 4.508(0.3); 3.902(1.0); 3.671(0.8); 3.645(0.6); 3.628(0.6); 3.604(0.3); 3.502(9.7); 3.501(9.6); 3.324(98.1); 2.675(0.4); 2.671(0.5); 2.666(0.4);
2.549(0.3); 2.541(0.5); 2.524(1.7); 2.511(32.0); 2.506(64.3); 2.502(84.1); 2.497(60.0); 2.493(28.7); 2.476(0.8); 2.351(0.9); 2.346(0.9); 2.333(1.1); 2.328(1.1); 2.324(1.1); 2.304(0.4);
2.300(0.4); 2.294(0.5); 2.120(0.3); 2.097(0.7); 2.074(0.4); 2.065(0.6); 2.047(0.5); 2.031(0.5); 2.021(0.5); 2.001(0.6); 1.904(16.0); 1.882(0.6); 1.868(0.6); 1.850(0.4); 1.605(0.5);
1.587(0.3); 1.577(0.4); 0.546(0.4); 0.536(0.7); 0.531(0.8); 0.514(1.3); 0.499(0.8); 0.484(0.5); 0.255(0.4); 0.234(0.7); 0.215(0.8); 0.205(0.8); 0.194(0.5); 0.000(7.4)
Example 165: ¹H-NMR(400.0 MHz. DMSO): δ = 8.315(0.7); 7.595(2.5); 7.395(1.5); 7.390(1.6); 7.373(2.1); 7.369(1.9); 7.253(0.4); 3.902(4.5); 3.646(0.3); 3.621(0.4); 3.516(16.0);
3.387(0.3); 3.379(0.4); 3.361(0.7); 3.325(394.9); 3.175(0.6); 3.162(0.6); 2.675(1.2); 2.671(1.6); 2.666(1.2); 2.563(0.4); 2.541(1.2); 2.524(4.5); 2.511(99.7); 2.506(198.7); 2.502(257.7);
2.497(181.4); 2.493(84.1); 2.337(0.5); 2.333(1.2); 2.329(1.6); 2.324(1.1); 2.319(0.5); 1.989(0.6); 1.973(0.7); 1.913(14.5); 1.825(2.2); 1.791(1.6); 1.751(1.3); 1.728(1.4); 1.458(0.7);
1.428(0.8); 1.426(0.8); 1.400(0.8); 1.372(1.0); 1.346(1.5); 1.322(1.4); 1.298(0.7); 0.708(0.7); 0.695(0.7); 0.492(0.9); 0.146(0.4); 0.008(0.9); 0.000(25.7); −0.009(0.7)
Example 166: ¹H-NMR(400.0 MHz. DMSO): δ = 8.315(0.4); 7.576(6.3); 7.570(6.6); 7.487(2.6); 7.465(4.6); 7.417(3.3); 7.411(3.0); 7.395(1.8); 7.390(1.7); 6.986(1.6); 6.851(3.4);
6.716(1.7); 4.437(0.7); 4.415(1.7); 4.389(2.0); 4.367(0.9); 4.127(0.7); 4.101(1.4); 4.081(1.4); 4.054(0.6); 3.902(2.2); 3.730(16.0); 3.324(197.7); 2.680(0.5); 2.675(0.9); 2.671(1.2);
2.666(0.9); 2.541(0.7); 2.524(3.2); 2.511(71.5); 2.506(141.2); 2.502(182.6); 2.497(129.5); 2.493(61.2); 2.434(0.7); 2.429(1.0); 2.418(1.3); 2.412(1.3); 2.402(1.0); 2.333(0.8); 2.329(1.1);
2.324(0.7); 2.320(0.3); 2.173(0.4); 2.162(0.5); 2.153(0.8); 2.143(1.1); 2.133(0.9); 2.123(1.2); 2.113(0.9); 2.102(0.6); 2.093(0.5); 2.081(0.5); 2.070(0.7); 2.057(0.9); 2.050(1.2);
2.036(1.5); 2.029(0.9); 2.016(1.7); 1.998(1.3); 1.977(0.8); 1.968(0.6); 1.947(0.4); 1.912(0.5); 1.900(0.6); 1.889(0.7); 1.881(1.0); 1.867(0.9); 1.860(0.9); 1.844(0.9); 1.831(0.9);
1.822(1.0); 1.813(0.9); 1.803(0.9); 1.793(0.6); 1.783(0.6); 1.626(0.5); 1.604(1.1); 1.578(1.2); 1.574(1.1); 1.547(0.9); 1.526(0.3); 0.663(0.5); 0.655(0.7); 0.645(1.1); 0.635(1.9);
0.624(2.0); 0.617(2.0); 0.597(0.8); 0.588(0.5); 0.395(0.6); 0.366(1.2); 0.321(1.4); 0.314(1.3); 0.299(0.8); 0.284(0.6); 0.008(0.6); 0.000(16.8); −0.009(0.5)
Example 167: ¹H-NMR(400.0 MHz. DMSO): δ = 8.315(0.4); 7.429(2.7); 7.423(1.7); 7.420(1.5); 7.416(1.4); 7.411(1.9); 7.406(3.3); 7.392(1.8); 7.387(1.3); 7.379(1.6); 7.369(2.2);
7.248(0.4); 7.242(0.9); 7.230(3.4); 7.225(4.1); 7.216(5.7); 7.206(3.4); 7.203(2.7); 7.190(0.6); 6.909(1.8); 6.774(3.9); 6.639(2.0); 4.564(0.6); 4.542(1.5); 4.522(1.5); 4.500(0.6);
3.902(2.3); 3.679(0.8); 3.659(16.0); 3.637(1.2); 3.611(0.7); 3.392(0.4); 3.328(298.1); 2.675(0.9); 2.671(1.2); 2.666(0.8); 2.662(0.4); 2.546(0.6); 2.541(1.0); 2.524(3.8); 2.511(72.8);
2.506(143.2); 2.502(184.9); 2.497(130.5); 2.493(61.6); 2.474(1.4); 2.365(0.9); 2.344(1.7); 2.338(1.8); 2.333(2.1); 2.329(2.0); 2.324(1.8); 2.319(1.3); 2.297(0.6); 2.292(0.7); 2.286(0.8);
2.266(0.4); 2.124(0.6); 2.101(1.2); 2.078(0.8); 2.069(0.9); 2.049(1.0); 2.030(0.9); 2.019(0.8); 2.000(1.0); 1.979(0.4); 1.913(0.5); 1.897(1.1); 1.881(0.9); 1.868(1.0); 1.850(0.5);
1.628(0.6); 1.606(0.9); 1.579(0.8); 1.556(0.4); 0.588(0.5); 0.568(1.6); 0.549(1.5); 0.529(1.6); 0.512(1.7); 0.488(0.5); 0.277(0.4); 0.249(3.2); 0.243(3.0); 0.008(0.6); 0.000(16.7); −0.009
(0.5)
Example 168: ¹H-NMR(400.0 MHz. DMSO): δ = 8.315(0.8); 7.603(2.7); 7.391(1.4); 7.373(1.7); 7.301(0.4); 7.266(0.4); 6.930(1.0); 6.796(2.0); 6.660(1.0); 4.101(0.3); 4.088(0.3);
3.902(5.4); 3.672(16.0); 3.594(0.4); 3.450(0.3); 3.428(0.4); 3.397(0.5); 3.389(0.7); 3.361(1.2); 3.326(465.7); 3.175(1.6); 3.162(1.5); 2.675(1.6); 2.671(2.1); 2.666(1.6); 2.541(1.7);
2.524(6.7); 2.511(126.2); 2.506(247.5); 2.502(319.3); 2.497(224.6); 2.493(106.0); 2.333(1.4); 2.329(1.9); 2.324(1.3); 2.320(0.6); 1.975(0.7); 1.824(2.3); 1.792(1.9); 1.751(1.4);
1.730(1.7); 1.457(0.8); 1.426(1.0); 1.401(0.9); 1.377(1.1); 1.351(1.6); 1.335(1.5); 0.713(0.8); 0.705(0.8); 0.669(0.6); 0.519(1.1); 0.177(0.3); 0.146(0.4); 0.008(0.9); 0.000(24.3); −0.009
(0.8)
Example 169: ¹H-NMR(400.0 MHz. DMSO): δ = 8.315(0.6); 7.450(2.5); 7.431(3.1); 7.281(2.1); 7.268(2.4); 7.253(3.5); 7.233(3.4); 7.211(1.4); 6.902(0.8); 6.766(1.7); 6.633(0.9);
3.902(3.3); 3.696(0.5); 3.647(16.0); 3.389(0.3); 3.383(0.3); 3.370(0.5); 3.363(0.5); 3.354(1.0); 3.325(451.0); 3.299(0.9); 3.290(0.6); 2.680(0.6); 2.675(1.1); 2.671(1.5); 2.666(1.1);
2.662(0.5); 2.559(0.3); 2.541(1.0); 2.524(4.2); 2.511(97.2); 2.506(193.4); 2.502(250.5); 2.497(178.1); 2.493(84.0); 2.333(1.1); 2.328(1.5); 2.324(1.1); 2.016(0.5); 1.978(1.0); 1.903(1.3);
1.874(1.6); 1.830(2.7); 1.799(2.2); 1.757(1.7); 1.736(2.0); 1.461(0.9); 1.430(1.2); 1.413(1.2); 1.387(1.3); 1.356(2.0); 1.330(1.8); 1.303(0.9); 1.274(0.4); 0.806(0.4); 0.699(1.0);
0.501(1.2); 0.146(0.5); 0.127(0.4); 0.008(0.7); 0.000(18.5); −0.009(0.7)
Example 170: ¹H-NMR(400.0 MHz. DMSO): δ = 8.315(0.4); 7.650(1.5); 7.442(1.2); 7.422(1.3); 6.701(0.4); 6.567(0.8); 6.432(0.4); 3.902(6.0); 3.324(104.8); 2.675(0.9); 2.671(1.3);
2.666(1.0); 2.631(16.0); 2.541(0.8); 2.524(3.4); 2.511(75.6); 2.506(148.9); 2.502(191.5); 2.497(134.3); 2.493(62.2); 2.333(0.9); 2.328(1.2); 2.324(0.8); 1.865(1.4); 1.835(1.7);
1.803(1.5); 1.745(1.2); 1.470(0.6); 1.437(0.8); 1.366(1.4); 0.854(0.4); 0.834(0.4); 0.813(0.4); 0.796(0.4); 0.789(0.4); 0.768(0.4); 0.499(0.4); 0.466(0.5); 0.000(10.0); −0.008(0.4)
Example 171: ¹H-NMR(400.0 MHz. DMSO): δ = 8.315(0.4); 7.497(1.7); 7.478(1.9); 7.331(1.8); 7.307(3.4); 7.288(3.0); 7.266(1.3); 6.580(0.4); 6.441(0.7); 6.315(0.4); 3.902(7.0);
3.654(0.3); 3.638(0.3); 3.321(155.4); 2.675(0.9); 2.670(1.3); 2.666(0.9); 2.610(16.0); 2.540(0.4); 2.524(3.2); 2.506(181.7); 2.501(233.4); 2.497(169.5); 2.380(0.4); 2.362(0.3);
2.332(1.3); 2.328(1.6); 2.324(1.2); 1.883(2.5); 1.845(2.3); 1.809(2.1); 1.748(1.7); 1.476(0.9); 1.446(1.0); 1.417(1.1); 1.394(1.8); 1.372(2.2); 1.311(0.3); 1.236(0.4); 0.854(0.6);
0.834(0.6); 0.813(0.6); 0.784(0.6); 0.767(0.6); 0.754(0.5); 0.732(0.4); 0.423(0.6); 0.403(0.6); 0.328(0.4); 0.272(0.5); 0.234(0.4); 0.000(10.3); −0.008(0.5)
Example 172: ¹H-NMR(400.0 MHz. DMSO): δ = 7.411(2.0); 7.400(2.9); 7.388(3.6); 7.374(1.8); 7.225(0.8); 7.214(4.0); 7.206(2.9); 7.202(3.5); 7.196(2.3); 7.190(3.3); 4.567(0.5);
4.545(1.3); 4.524(1.3); 4.502(0.5); 3.902(1.1); 3.660(0.6); 3.635(1.1); 3.618(1.1); 3.593(0.6); 3.510(16.0); 3.322(100.5); 2.671(0.9); 2.576(0.4); 2.540(1.3); 2.506(120.7); 2.502(140.9);
2.497(100.3); 2.371(2.5); 2.352(5.9); 2.333(6.4); 2.314(2.7); 2.291(0.7); 2.284(0.8); 2.263(0.5); 2.122(0.6); 2.100(1.2); 2.069(1.0); 2.048(0.9); 2.030(0.9); 2.018(0.8); 2.002(1.0);
1.981(0.5); 1.904(0.5); 1.885(1.1); 1.870(0.9); 1.857(1.0); 1.840(0.5); 1.622(0.6); 1.600(0.8); 1.573(0.8); 1.553(0.4); 0.999(5.7); 0.980(11.7); 0.961(5.3); 0.566(0.5); 0.547(1.5);
0.525(1.4); 0.514(1.4); 0.494(1.5); 0.471(0.5); 0.228(3.5); 0.000(4.7)
Example 173: ¹H-NMR(400.0 MHz. DMSO): δ = 7.596(3.3); 7.591(3.4); 7.537(1.4); 7.516(2.1); 7.447(1.8); 7.442(1.6); 7.426(1.2); 7.420(1.1); 6.954(1.0); 6.819(2.3); 6.684(1.1);
4.369(0.4); 4.346(0.9); 4.321(1.1); 4.299(0.5); 4.150(0.4); 4.124(0.8); 4.104(0.8); 4.078(0.3); 3.902(2.1); 3.321(76.9); 2.677(16.0); 2.541(0.4); 2.510(43.6); 2.506(82.4); 2.502(104.1);
2.497(73.2); 2.493(34.3); 2.395(0.6); 2.387(0.7); 2.378(1.1); 2.369(0.8); 2.361(0.6); 2.333(0.5); 2.328(0.6); 2.324(0.5); 2.130(0.5); 2.121(0.7); 2.109(0.7); 2.101(1.3); 2.083(2.1);
2.063(2.0); 2.044(0.9); 1.918(0.4); 1.908(0.6); 1.887(0.7); 1.868(0.6); 1.846(0.5); 1.837(0.5); 1.827(0.5); 1.817(0.6); 1.807(0.4); 1.665(0.6); 1.639(0.7); 1.636(0.7); 1.609(0.5);
0.626(0.4); 0.609(0.8); 0.601(1.0); 0.592(1.0); 0.585(1.0); 0.576(0.8); 0.560(0.4); 0.438(0.4); 0.425(0.6); 0.414(0.8); 0.399(0.7); 0.388(0.4); 0.361(0.6); 0.352(0.5); 0.000(5.0)
Example 174: ¹H-NMR(400.0 MHz. DMSO): δ = 7.488(1.5); 7.484(1.7); 7.469(1.5); 7.465(1.8); 7.403(1.0); 7.398(1.0); 7.385(1.3); 7.380(1.3); 7.308(0.5); 7.303(0.7); 7.290(1.6);
7.285(1.4); 7.271(2.6); 7.266(2.5); 7.252(1.4); 7.248(1.2); 7.233(0.4); 7.230(0.4); 6.710(1.0); 6.576(2.4); 6.443(1.2); 4.574(0.4); 4.553(0.9); 4.531(0.9); 4.509(0.4); 3.902(1.1);
3.684(0.4); 3.667(0.5); 3.659(0.6); 3.643(0.6); 3.633(0.5); 3.615(0.4); 3.322(61.7); 2.675(0.5); 2.671(0.6); 2.666(0.4); 2.599(16.0); 2.550(0.6); 2.541(0.5); 2.531(0.9); 2.524(2.1);
2.511(38.2); 2.506(75.0); 2.502(96.2); 2.497(67.9); 2.492(31.8); 2.393(0.4); 2.383(0.3); 2.373(0.3); 2.363(0.6); 2.354(0.4); 2.333(0.9); 2.328(0.8); 2.324(0.6); 2.318(0.4); 2.313(0.5);
2.287(0.7); 2.143(0.3); 2.121(0.7); 2.112(0.4); 2.098(0.5); 2.090(0.6); 2.065(0.5); 2.040(0.5); 2.027(0.5); 2.010(0.6); 1.904(0.7); 1.889(0.6); 1.875(0.6); 1.859(0.3); 1.627(0.3);
1.607(0.5); 1.597(0.4); 1.587(0.4); 1.579(0.4); 0.631(0.4); 0.626(0.4); 0.608(0.8); 0.591(0.5); 0.584(0.5); 0.475(0.5); 0.468(0.5); 0.458(0.7); 0.452(0.6); 0.440(0.4); 0.435(0.4);
0.366(2.0); 0.000(5.3)
Example 175: ¹H-NMR(400.0 MHz. DMSO): δ = 9.455(0.7); 8.315(0.8); 7.625(0.4); 7.619(0.4); 7.571(4.0); 7.566(3.9); 7.544(1.5); 7.539(1.5); 7.520(0.8); 7.505(0.9); 7.494(0.7);
7.483(1.4); 7.462(1.2); 7.454(0.9); 7.442(1.4); 7.419(6.2); 7.415(4.4); 7.403(1.2); 7.397(1.7); 7.375(0.7); 7.011(1.3); 6.215(0.5); 6.001(3.2); 4.451(0.7); 4.429(1.4); 4.403(1.6);
4.381(0.9); 4.288(0.4); 4.273(0.5); 4.133(0.4); 4.125(0.4); 4.096(0.9); 4.071(1.4); 4.051(1.3); 4.024(0.7); 3.902(11.8); 3.364(0.3); 3.322(263.6); 3.286(0.4); 2.675(1.5); 2.671(2.1);
2.666(1.5); 2.662(0.7); 2.541(1.0); 2.524(5.9); 2.511(147.6); 2.506(292.4); 2.502(379.5); 2.497(272.4); 2.493(131.5); 2.435(1.0); 2.431(1.0); 2.425(0.9); 2.421(0.9); 2.402(1.1);
2.392(1.4); 2.385(1.5); 2.375(1.8); 2.365(3.0); 2.348(0.8); 2.333(2.2); 2.328(2.8); 2.324(2.2); 2.319(1.3); 2.309(2.0); 2.279(2.7); 2.260(0.6); 2.257(0.7); 2.237(3.4); 2.225(2.6);
2.210(1.1); 2.199(0.8); 2.167(13.0); 2.146(4.1); 2.140(2.2); 2.130(1.9); 2.120(2.2); 2.098(16.0); 2.067(1.7); 2.059(1.6); 2.034(2.2); 2.028(2.2); 2.013(3.0); 1.995(2.4); 1.973(1.3);
1.960(1.1); 1.943(0.9); 1.927(0.9); 1.908(2.0); 1.899(1.4); 1.884(1.5); 1.877(1.7); 1.866(1.8); 1.845(1.8); 1.826(1.8); 1.812(1.9); 1.804(1.9); 1.793(1.9); 1.787(1.7); 1.764(1.2);
1.752(1.1); 1.718(0.5); 1.711(0.5); 1.701(0.5); 1.695(0.4); 1.690(0.4); 1.668(0.7); 1.629(0.6); 1.619(0.6); 1.601(0.7); 1.587(1.0); 1.559(1.6); 1.539(1.4); 1.519(1.0); 1.508(1.1);
1.485(0.8); 1.467(0.6); 1.453(0.7); 1.449(0.6); 1.433(0.7); 1.417(0.4); 1.379(0.4); 1.368(0.4); 1.323(0.3); 1.298(0.5); 1.259(0.5); 1.238(0.6); 0.853(0.5); 0.829(0.5); 0.826(0.5);
0.818(0.6); 0.813(0.5); 0.789(0.4); 0.755(0.5); 0.732(0.6); 0.704(0.7); 0.679(0.7); 0.673(0.8); 0.657(0.8); 0.634(1.3); 0.617(1.7); 0.605(1.8); 0.592(2.0); 0.579(1.7); 0.563(1.3);
0.557(1.1); 0.519(0.4); 0.486(0.4); 0.471(0.4); 0.459(0.4); 0.447(0.4); 0.408(1.1); 0.398(1.2); 0.386(1.2); 0.344(0.6); 0.322(0.9); 0.311(1.3); 0.298(1.4); 0.288(1.1); 0.276(0.7);
0.271(0.8); 0.260(0.5); 0.008(0.7); 0.000(17.3); −0.009(0.7)
Example 176: ¹H-NMR(400.0 MHz. DMSO): δ = 9.394(1.0); 8.896(0.4); 8.315(1.4); 7.743(0.3); 7.646(0.4); 7.639(0.4); 7.631(0.6); 7.609(0.3); 7.544(0.5); 7.529(0.9); 7.511(0.6);
7.488(0.7); 7.471(1.0); 7.461(1.1); 7.449(1.6); 7.425(2.9); 7.419(2.9); 7.402(3.2); 7.379(2.1); 7.375(2.0); 7.360(1.9); 7.356(2.1); 7.336(1.2); 7.314(1.2); 7.299(1.3); 7.290(1.5);
7.276(1.9); 7.268(2.0); 7.263(2.1); 7.257(2.2); 7.251(2.2); 7.242(2.6); 7.238(2.7); 7.230(3.7); 7.223(3.2); 7.220(3.2); 7.207(3.1); 7.194(1.7); 7.188(1.7); 6.917(2.0); 6.493(0.3);
5.487(1.0); 4.566(0.4); 4.560(0.5); 4.541(0.7); 4.517(0.6); 4.485(0.4); 4.461(0.3); 4.447(0.4); 4.400(0.4); 4.395(0.4); 4.378(0.4); 4.357(0.4); 4.346(0.4); 4.340(0.4); 4.256(0.4);
4.242(0.3); 4.214(0.4); 4.202(0.4); 3.902(16.0); 3.692(0.3); 3.669(0.5); 3.654(0.6); 3.642(0.9); 3.625(0.7); 3.605(0.4); 3.590(0.4); 3.565(0.5); 3.322(313.0); 3.252(0.6); 3.245(0.5);
3.223(0.4); 2.961(0.5); 2.680(0.7); 2.675(1.9); 2.671(2.9); 2.666(1.9); 2.661(0.6); 2.524(2.4); 2.511(209.2); 2.506(432.1); 2.502(574.5); 2.497(419.8); 2.492(208.8); 2.417(2.9);
2.405(2.7); 2.399(2.6); 2.386(2.7); 2.363(2.6); 2.333(4.6); 2.328(5.5); 2.324(4.6); 2.319(3.3); 2.309(2.4); 2.304(2.4); 2.293(3.9); 2.284(2.6); 2.272(2.3); 2.256(7.9); 2.234(2.5);
2.218(1.9); 2.191(2.0); 2.169(3.8); 2.142(1.9); 2.138(2.0); 2.123(3.1); 2.112(2.4); 2.101(4.9); 2.096(6.4); 2.065(5.3); 2.053(3.1); 2.032(5.4); 2.015(2.9); 2.005(3.6); 1.963(7.2);
1.940(2.0); 1.931(1.8); 1.925(2.2); 1.908(2.1); 1.890(2.0); 1.880(1.9); 1.827(1.3); 1.810(1.3); 1.791(1.5); 1.773(1.1); 1.765(1.1); 1.748(1.0); 1.721(0.9); 1.703(1.0); 1.694(0.9);
1.684(0.8); 1.662(1.0); 1.636(1.2); 1.621(1.4); 1.574(2.2); 1.554(1.4); 1.544(1.5); 1.528(1.3); 1.485(2.0); 1.467(1.4); 1.421(1.0); 1.388(0.7); 1.372(0.7); 1.334(0.8); 1.315(0.7);
1.298(0.8); 1.284(0.7); 1.274(0.7); 1.258(0.9); 1.235(1.0); 1.221(0.6); 1.182(0.5); 1.155(0.5); 1.113(0.4); 1.066(0.4); 1.046(0.4); 1.029(0.4); 1.002(0.4); 0.980(0.4); 0.956(0.5);
0.936(0.4); 0.922(0.4); 0.911(0.4); 0.887(0.5); 0.873(0.6); 0.854(0.8); 0.835(0.8); 0.762(0.6); 0.754(0.6); 0.740(0.6); 0.716(0.7); 0.669(1.0); 0.656(1.1); 0.654(1.1); 0.639(1.1);
0.611(1.1); 0.578(1.2); 0.568(1.2); 0.559(1.2); 0.550(1.3); 0.528(1.4); 0.522(1.4); 0.515(1.3); 0.492(1.6); 0.482(1.6); 0.476(1.6); 0.472(1.5); 0.455(1.3); 0.431(1.5); 0.414(1.4);
0.404(0.9); 0.396(0.8); 0.336(0.7); 0.320(0.8); 0.272(1.0); 0.230(0.7); 0.202(0.9); 0.188(0.9); 0.176(0.8); 0.155(0.6); 0.147(0.6); 0.134(0.6); 0.080(0.4); 0.008(1.1); 0.000(31.8); −0.009
(1.4); −0.035(0.5); −0.040(0.5); −0.062(0.5); −0.070(0.4); −0.079(0.4)
Example 177: ¹H-NMR(400.0 MHz. DMSO): δ = 8.315(0.9); 7.458(3.0); 7.439(3.5); 7.284(3.2); 7.267(3.3); 7.249(4.0); 7.229(4.4); 7.207(2.2); 5.515(1.2); 4.654(0.4); 4.592(0.4);
3.902(5.3); 3.636(0.7); 3.323(237.1); 3.167(0.4); 2.675(1.7); 2.671(2.3); 2.541(1.9); 2.506(316.6); 2.502(394.2); 2.497(290.9); 2.333(2.3); 2.328(2.7); 2.295(0.7); 2.264(0.5); 2.193(0.5);
2.105(16.0); 2.042(1.0); 1.872(8.9); 1.830(4.8); 1.800(3.9); 1.751(3.0); 1.729(3.0); 1.567(0.4); 1.505(0.5); 1.405(2.2); 1.375(2.5); 1.344(2.7); 1.313(2.3); 1.281(1.4); 1.260(1.0);
0.694(1.7); 0.451(1.5); 0.147(0.8); 0.000(22.5)
Example 178: ¹H-NMR(400.0 MHz. DMSO): δ = 8.516(2.2); 8.506(2.1); 8.315(0.3); 7.291(2.3); 7.273(6.1); 7.254(4.6); 7.189(2.2); 7.170(3.2); 7.152(6.3); 7.149(6.4); 7.132(4.6);
3.902(1.6); 3.820(16.0); 3.355(0.4); 3.324(184.9); 2.975(0.5); 2.965(1.3); 2.955(1.8); 2.945(1.8); 2.936(1.3); 2.926(0.5); 2.675(0.7); 2.670(0.9); 2.666(0.7); 2.541(1.0); 2.510(61.8);
2.506(118.2); 2.502(149.8); 2.497(106.2); 2.493(50.3); 2.333(0.6); 2.328(0.9); 2.324(0.7); 2.034(0.9); 2.025(1.0); 2.018(1.2); 2.010(2.0); 2.002(1.2); 1.995(1.1); 1.986(1.0); 1.269(0.8);
1.256(2.0); 1.244(1.9); 1.232(2.5); 1.220(1.8); 1.214(2.5); 1.211(2.1); 1.195(2.1); 1.180(0.7); 0.000(4.7)
Example 179: ¹H-NMR(400.0 MHz. DMSO): δ = 8.315(0.4); 7.943(2.2); 7.935(2.2); 7.266(5.5); 7.261(2.1); 7.249(3.1); 7.245(10.2); 7.195(9.0); 7.174(4.8); 6.978(2.1); 6.844(4.5);
6.710(2.3); 4.099(0.4); 3.902(2.4); 3.723(16.0); 3.435(0.5); 3.358(0.5); 3.324(297.7); 3.118(0.5); 3.106(0.9); 3.098(1.3); 3.088(1.7); 3.079(1.3); 3.071(0.9); 3.059(0.5); 2.675(0.9);
2.671(1.3); 2.666(1.0); 2.541(0.8); 2.524(4.0); 2.510(93.1); 2.506(177.7); 2.502(225.1); 2.497(160.1); 2.493(76.6); 2.333(1.1); 2.328(1.4); 2.324(1.0); 2.320(0.6); 2.291(0.9); 2.273(1.9);
2.251(1.9); 2.233(0.9); 1.348(1.0); 1.332(1.6); 1.328(1.5); 1.310(1.9); 1.305(1.3); 1.290(1.2); 1.250(1.3); 1.234(2.1); 1.220(2.0); 1.205(0.9); 0.000(5.8)
Example 180: ¹H-NMR(400.0 MHz. DMSO): δ = 8.315(6.7); 7.566(5.2); 7.547(5.9); 7.456(3.5); 7.436(4.5); 7.312(3.5); 7.307(4.4); 7.293(6.7); 7.288(8.4); 7.274(4.4); 7.268(5.0);
7.211(3.0); 7.209(3.0); 7.206(2.9); 7.193(4.4); 7.188(4.1); 7.175(1.5); 7.172(1.5); 7.169(1.4); 6.978(2.0); 6.843(4.3); 6.708(2.3); 6.657(0.4); 4.734(0.4); 4.718(0.5); 4.711(0.6);
4.698(0.6); 4.573(0.4); 3.902(8.6); 3.818(0.5); 3.701(16.0); 3.671(0.7); 3.644(13.7); 3.326(963.6); 2.675(2.6); 2.671(3.6); 2.666(2.6); 2.662(1.2); 2.541(1.9); 2.524(8.7); 2.511(220.4);
2.506(446.9); 2.502(585.5); 2.497(418.1); 2.493(198.0); 2.419(0.9); 2.384(0.8); 2.337(1.6); 2.333(2.9); 2.329(3.9); 2.324(2.9); 2.320(1.5); 2.177(0.8); 2.162(0.8); 2.148(0.9); 2.081(1.0);
2.066(1.0); 1.994(1.7); 1.982(1.8); 1.981(1.8); 1.921(1.0); 1.899(0.9); 1.865(2.6); 1.842(3.9); 1.833(3.5); 1.809(2.2); 1.743(1.1); 1.734(1.2); 1.710(1.5); 1.701(1.1); 1.662(11.8);
1.634(1.6); 1.602(1.9); 1.593(1.7); 1.543(4.2); 1.509(2.5); 1.480(1.1); 1.462(2.0); 1.428(1.5); 1.372(15.3); 1.337(0.5); 1.298(0.5); 1.285(0.9); 1.259(0.6); 1.249(0.5); 1.236(1.0);
1.226(0.5); 0.855(0.3); 0.852(0.3); 0.619(1.6); 0.486(1.0); 0.475(1.1); 0.461(1.0); 0.432(1.0); 0.419(1.4); 0.397(1.5); 0.381(1.5); 0.366(1.2); 0.350(0.6); 0.331(0.7); 0.315(1.3);
0.303(1.4); 0.297(1.3); 0.291(1.1); 0.286(1.0); 0.280(0.9); 0.263(0.4); 0.145(1.0); 0.135(1.0); 0.034(0.9); 0.019(1.1); 0.008(2.1); 0.000(34.7); −0.008(1.8)
Example 181: ¹H-NMR(400.0 MHz. DMSO): δ = 8.316(0.4); 7.681(0.7); 7.660(0.9); 7.614(1.8); 7.592(1.8); 7.538(1.0); 7.517(0.7); 7.277(0.5); 7.224(12.7); 7.213(16.0); 7.152(0.4);
7.143(1.1); 7.132(2.1); 7.121(2.1); 7.110(1.2); 7.100(0.5); 6.932(1.7); 6.797(3.3); 6.662(1.8); 3.991(0.9); 3.968(1.0); 3.942(0.5); 3.903(1.4); 3.694(15.5); 3.326(136.3); 3.323(119.9);
2.879(0.6); 2.873(0.5); 2.673(1.8); 2.652(1.5); 2.645(1.4); 2.623(0.9); 2.615(0.7); 2.505(174.5); 2.502(186.8); 2.329(1.3); 1.946(1.3); 1.834(0.8); 1.817(2.1); 1.782(2.2); 1.764(1.9);
1.721(1.3); 1.583(0.4); 1.552(1.1); 1.520(1.2); 1.488(0.6); 1.446(0.7); 1.422(2.0); 1.401(2.3); 1.378(1.1); 1.354(0.7); 1.324(0.9); 1.294(0.7); 1.046(6.7); 0.001(29.4)
Example 182: ¹H-NMR(400.0 MHz. DMSO): δ = 8.315(0.6); 7.517(1.5); 7.495(1.5); 7.244(16.0); 7.234(9.2); 7.232(9.6); 7.217(0.6); 7.212(1.0); 7.165(1.1); 7.157(1.4); 7.153(1.2);
7.144(1.7); 7.135(0.9); 7.122(0.5); 6.947(2.0); 6.813(3.9); 6.679(2.2); 4.358(1.0); 4.350(1.1); 4.336(1.1); 4.328(1.0); 3.902(2.6); 3.746(14.3); 3.323(188.2); 2.947(0.7); 2.938(1.4);
2.930(0.8); 2.915(0.9); 2.906(1.5); 2.897(0.8); 2.675(0.8); 2.671(1.1); 2.666(0.8); 2.541(0.6); 2.524(2.9); 2.510(71.1); 2.506(140.3); 2.502(181.6); 2.497(129.5); 2.493(61.5); 2.333(0.9);
2.328(1.2); 2.324(0.9); 2.030(0.3); 2.006(0.8); 1.997(0.9); 1.974(1.0); 1.965(0.9); 1.941(0.4); 1.933(0.4); 1.841(1.6); 1.834(1.5); 1.809(2.1); 1.718(0.4); 1.676(1.7); 1.657(0.9);
1.648(1.2); 1.642(1.2); 1.548(1.2); 1.527(2.3); 1.499(0.7); 1.458(0.4); 1.449(0.4); 1.427(0.7); 1.419(0.6); 1.408(0.6); 1.400(0.7); 1.387(0.5); 1.378(0.5); 0.008(1.0); 0.000(24.3); −0.009
(0.8)
Example 183: ¹H-NMR(400.0 MHz. DMSO): δ = 8.315(0.5); 7.685(1.2); 7.663(1.2); 7.283(1.0); 7.279(1.6); 7.262(6.2); 7.257(8.1); 7.239(4.7); 7.227(0.5); 7.219(1.6); 7.172(0.9);
7.168(1.4); 7.163(0.8); 7.157(0.8); 7.151(1.7); 7.144(0.5); 7.138(0.5); 7.134(0.7); 7.129(0.4); 6.877(1.7); 6.742(3.5); 6.608(1.9); 4.422(0.8); 4.414(0.9); 4.400(0.9); 4.392(0.8);
3.902(1.6); 3.826(16.0); 3.322(160.6); 2.959(0.6); 2.950(1.1); 2.942(0.6); 2.926(0.7); 2.918(1.1); 2.909(0.6); 2.675(0.7); 2.671(1.0); 2.666(0.7); 2.661(0.3); 2.541(0.6); 2.524(2.6);
2.510(61.3); 2.506(122.9); 2.501(159.6); 2.497(112.7); 2.492(52.3); 2.337(0.4); 2.333(0.7); 2.328(1.0); 2.324(0.7); 2.319(0.3); 2.037(0.7); 2.028(0.7); 2.005(0.8); 1.997(0.7); 1.972(0.4);
1.861(0.7); 1.837(1.6); 1.810(0.8); 1.698(1.1); 1.689(1.3); 1.680(1.1); 1.671(0.9); 1.664(1.0); 1.657(0.9); 1.638(0.4); 1.542(1.1); 1.524(1.3); 1.491(0.4); 1.484(0.6); 1.450(0.4);
1.423(0.5); 1.414(0.6); 1.393(0.4); 1.385(0.5); 0.008(1.1); 0.000(34.3); −0.009(1.1)
Example 184: ¹H-NMR(400.0 MHz. DMSO): δ = 8.315(0.6); 7.804(1.3); 7.782(1.3); 7.238(9.1); 7.227(16.0); 7.216(0.7); 7.161(1.0); 7.150(1.5); 7.139(1.6); 7.131(0.6); 7.127(0.9);
7.117(0.4); 6.727(1.5); 6.592(2.6); 6.458(1.7); 4.023(0.7); 4.013(0.5); 4.001(0.6); 3.992(0.6); 3.973(0.3); 3.902(2.2); 3.768(14.7); 3.322(184.5); 2.675(1.1); 2.670(1.5); 2.666(1.2);
2.661(0.9); 2.651(0.5); 2.630(1.0); 2.622(0.9); 2.602(0.6); 2.593(0.5); 2.541(0.7); 2.524(4.4); 2.510(97.3); 2.506(190.5); 2.501(245.9); 2.497(174.7); 2.492(82.6); 2.333(1.2); 2.328(1.6);
2.323(1.2); 1.988(0.8); 1.959(0.7); 1.824(0.7); 1.794(1.4); 1.761(1.2); 1.717(0.8); 1.536(0.8); 1.528(0.7); 1.504(0.9); 1.497(0.8); 1.465(0.6); 1.425(0.8); 1.403(1.1); 1.380(1.0);
1.355(0.8); 1.328(0.8); 1.297(0.6); 0.008(3.0); 0.000(74.5); −0.009(2.5); −0.150(0.3)
Example 185: ¹H-NMR(400.0 MHz. DMSO): δ = 8.315(0.4); 8.011(3.8); 7.864(1.5); 7.842(1.5); 7.264(1.5); 7.238(0.7); 7.233(1.3); 7.228(0.8); 7.214(9.3); 7.198(4.5); 7.178(1.3);
7.128(3.2); 7.111(0.8); 7.106(1.3); 7.101(0.7); 7.090(1.5); 7.078(0.5); 7.073(0.7); 7.069(0.4); 6.992(1.6); 4.030(0.7); 4.020(0.6); 4.007(0.7); 3.980(0.3); 3.902(1.4); 3.837(16.0);
3.323(118.8); 2.675(0.6); 2.671(0.8); 2.666(0.6); 2.657(0.5); 2.649(0.6); 2.628(1.1); 2.620(1.0); 2.599(0.6); 2.590(0.6); 2.541(0.4); 2.524(2.0); 2.511(50.3); 2.506(99.9); 2.502(129.3);
2.497(91.6); 2.493(43.2); 2.333(0.6); 2.328(0.8); 2.324(0.6); 1.929(0.8); 1.903(0.7); 1.830(0.7); 1.794(1.4); 1.762(1.5); 1.727(0.8); 1.525(0.8); 1.518(0.6); 1.493(0.8); 1.461(0.4);
1.442(0.5); 1.411(0.8); 1.391(1.0); 1.369(1.5); 1.337(1.2); 1.305(0.6); 0.008(1.1); 0.000(31.7); −0.009(1.1)
Example 186: ¹H-NMR(400.0 MHz. DMSO): δ = 8.385(3.9); 8.315(0.4); 7.487(1.4); 7.464(1.4); 7.255(1.2); 7.251(1.8); 7.234(6.1); 7.230(5.4); 7.227(6.0); 7.221(1.0); 7.209(4.7);
7.205(1.7); 7.198(2.1); 7.193(1.2); 7.189(1.7); 7.129(1.0); 7.125(1.5); 7.120(0.8); 7.113(0.8); 7.108(1.8); 7.102(0.6); 7.095(0.5); 7.091(0.9); 7.086(0.5); 7.062(3.5); 6.926(1.8);
4.418(0.8); 4.410(0.9); 4.396(0.8); 4.387(0.8); 3.897(16.0); 3.837(0.9); 3.323(110.8); 3.175(0.9); 3.162(0.9); 2.939(0.6); 2.930(1.0); 2.922(0.6); 2.906(0.7); 2.897(1.1); 2.889(0.6);
2.680(0.3); 2.675(0.7); 2.671(0.9); 2.666(0.7); 2.541(0.5); 2.524(2.4); 2.510(56.6); 2.506(113.7); 2.501(147.8); 2.497(103.9); 2.492(48.0); 2.337(0.3); 2.333(0.7); 2.328(0.9); 2.324(0.7);
2.193(0.7); 2.184(0.7); 2.161(0.8); 2.152(0.7); 1.860(0.7); 1.828(0.8); 1.775(0.5); 1.768(0.5); 1.743(1.0); 1.737(1.1); 1.716(0.4); 1.704(0.5); 1.695(0.5); 1.687(0.5); 1.675(0.8);
1.666(0.7); 1.641(1.1); 1.624(0.6); 1.615(0.8); 1.604(0.9); 1.595(0.7); 1.570(0.6); 1.561(0.8); 1.535(1.3); 1.461(0.4); 1.439(0.5); 1.429(0.7); 1.420(0.5); 1.409(0.5); 1.398(0.6);
0.008(1.8); 0.000(51.9); −0.009(1.6)
Example 187: ¹H-NMR(400.0 MHz. DMSO): δ = 8.315(0.5); 8.253(2.3); 8.244(2.3); 7.331(0.9); 7.314(1.4); 7.308(1.6); 7.301(0.8); 7.295(1.5); 7.290(1.8); 7.281(0.4); 7.273(1.2);
7.261(2.1); 7.126(4.6); 7.018(3.0); 7.000(5.0); 6.996(4.9); 6.989(3.5); 6.982(2.3); 6.974(1.7); 6.966(3.1); 3.902(1.7); 3.786(16.0); 3.323(179.6); 2.983(0.6); 2.973(1.3); 2.963(1.8);
2.953(1.8); 2.944(1.3); 2.933(0.6); 2.679(0.5); 2.675(1.0); 2.671(1.3); 2.666(0.9); 2.541(0.9); 2.524(4.0); 2.510(79.3); 2.506(153.5); 2.501(197.4); 2.497(140.2); 2.493(66.4); 2.333(0.9);
2.328(1.2); 2.324(0.9); 2.090(0.9); 2.082(1.0); 2.074(1.2); 2.066(1.9); 2.058(1.2); 2.051(1.1); 2.043(1.0); 1.354(1.0); 1.340(1.9); 1.328(1.8); 1.317(1.8); 1.304(1.2); 1.276(1.4);
1.261(2.3); 1.257(1.9); 1.246(1.4); 1.241(2.2); 1.227(0.9); 0.008(1.9); 0.000(43.0); −0.009(1.5)
Example 188: ¹H-NMR(400.0 MHz. DMSO): δ = 8.315(0.5); 8.233(2.3); 8.224(2.3); 7.265(2.1); 7.219(2.8); 7.214(1.4); 7.205(3.4); 7.198(4.5); 7.189(1.9); 7.184(4.0); 7.130(4.7);
7.117(4.8); 7.112(1.5); 7.095(7.6); 7.089(1.7); 7.078(1.2); 7.073(3.1); 6.996(2.2); 3.902(1.8); 3.785(16.0); 3.325(203.5); 2.909(0.5); 2.899(1.3); 2.889(1.9); 2.880(1.9); 2.870(1.3);
2.860(0.6); 2.675(0.9); 2.671(1.2); 2.666(0.9); 2.541(0.7); 2.524(3.0); 2.511(73.9); 2.506(147.3); 2.502(191.1); 2.497(136.7); 2.493(65.1); 2.333(0.9); 2.328(1.2); 2.324(0.9); 2.319(0.4);
2.072(0.9); 2.063(1.0); 2.056(1.2); 2.048(1.9); 2.040(1.2); 2.033(1.1); 2.024(1.0); 1.298(1.0); 1.284(1.8); 1.273(1.9); 1.261(1.8); 1.248(1.2); 1.208(1.3); 1.193(2.2); 1.189(1.8);
1.178(1.4); 1.173(2.1); 1.159(0.9); 0.008(0.9); 0.000(27.8); −0.008(1.0)
Example 189: ¹H-NMR(400.0 MHz. DMSO): δ = 8.315(0.6); 8.249(2.3); 8.237(2.5); 7.687(2.8); 7.668(3.3); 7.617(1.2); 7.598(2.9); 7.579(1.8); 7.417(1.7); 7.398(2.9); 7.379(1.4);
7.311(2.8); 7.292(2.6); 7.242(2.0); 7.107(4.5); 6.973(2.1); 3.902(2.3); 3.787(16.0); 3.324(272.4); 3.270(0.6); 3.258(1.3); 3.249(1.8); 3.237(1.9); 3.229(1.6); 3.216(0.7); 2.680(0.5);
2.675(1.0); 2.671(1.5); 2.666(1.3); 2.541(0.9); 2.524(3.3); 2.519(5.3); 2.511(74.4); 2.506(167.4); 2.502(242.6); 2.497(205.9); 2.493(130.8); 2.337(0.4); 2.333(1.0); 2.328(1.5);
2.324(1.3); 2.255(1.6); 1.446(0.9); 1.432(1.9); 1.420(1.9); 1.409(2.0); 1.396(1.3); 1.345(1.1); 1.329(2.0); 1.325(2.0); 1.310(2.2); 1.295(1.0); 0.008(0.9); 0.000(31.2); −0.008(3.9)
Example 190: ¹H-NMR(400.0 MHz. DMSO): δ = 8.315(1.6); 7.670(1.9); 7.650(2.0); 7.531(0.4); 7.517(0.4); 7.497(0.4); 7.473(0.4); 7.412(2.3); 7.394(3.0); 7.357(1.9); 7.338(3.5);
7.319(3.5); 7.306(3.4); 7.286(2.0); 7.250(1.6); 7.233(2.7); 7.213(1.9); 7.164(1.1); 7.141(1.1); 7.029(0.6); 7.005(0.6); 5.901(0.8); 5.888(0.7); 5.816(0.9); 5.788(0.9); 3.902(10.0);
3.808(0.5); 3.798(0.8); 3.759(0.4); 3.694(6.9); 3.670(0.9); 3.664(0.4); 3.585(6.7); 3.322(434.3); 2.679(1.3); 2.675(2.8); 2.670(3.9); 2.666(2.7); 2.661(1.2); 2.541(2.0); 2.524(10.3);
2.510(230.8); 2.506(462.6); 2.501(603.5); 2.497(427.1); 2.492(199.1); 2.458(0.9); 2.453(0.9); 2.422(0.8); 2.416(0.8); 2.390(0.7); 2.365(0.5); 2.337(1.5); 2.333(3.0); 2.328(4.0);
2.324(2.9); 2.319(1.5); 2.308(0.4); 2.277(1.1); 2.179(0.6); 2.150(1.1); 2.127(1.2); 2.097(0.6); 2.089(0.6); 1.974(1.0); 1.944(1.4); 1.917(1.5); 1.909(1.5); 1.895(1.2); 1.856(2.0);
1.823(16.0); 1.774(1.8); 1.733(0.7); 1.634(2.2); 1.604(2.2); 1.555(2.6); 1.514(2.5); 1.485(1.9); 1.459(7.4); 1.417(0.4); 1.411(0.4); 1.364(0.4); 1.284(0.3); 1.236(1.0); 1.024(1.0);
1.019(1.0); 1.011(1.0); 0.853(0.9); 0.836(1.1); 0.777(0.6); 0.764(0.6); 0.508(0.9); 0.423(0.8); 0.407(0.6); 0.146(1.0); 0.092(1.1); 0.075(1.5); 0.061(1.5); 0.045(1.3); 0.018(0.8);
0.008(7.4); 0.000(201.3); −0.009(7.5); −0.150(0.9)
Example 191: ¹H-NMR(400.0 MHz. DMSO): δ = 8.404(1.8); 8.394(1.7); 8.315(0.6); 7.339(0.6); 7.335(0.8); 7.317(1.1); 7.313(1.2); 7.300(1.1); 7.294(1.3); 7.284(0.4); 7.278(0.7);
7.222(1.7); 7.206(0.9); 7.087(3.9); 7.072(0.5); 7.023(2.3); 7.004(3.7); 6.994(2.6); 6.987(1.5); 6.977(1.2); 6.971(2.6); 6.953(1.9); 3.902(2.2); 3.888(5.6); 3.878(16.0); 3.324(182.1);
3.022(0.5); 3.011(1.0); 3.002(1.4); 2.991(1.4); 2.983(1.0); 2.971(0.4); 2.675(0.9); 2.671(1.1); 2.666(0.8); 2.662(0.4); 2.541(0.7); 2.524(3.4); 2.511(70.9); 2.506(137.3); 2.502(175.7);
2.497(124.1); 2.493(58.4); 2.333(0.8); 2.328(1.1); 2.324(0.8); 2.089(0.7); 2.081(0.7); 2.073(0.9); 2.066(1.4); 2.058(0.9); 2.050(0.8); 2.042(0.7); 1.328(0.6); 1.315(1.5); 1.302(1.4);
1.291(1.7); 1.287(1.6); 1.279(1.3); 1.271(1.9); 1.268(1.6); 1.256(1.0); 1.252(1.7); 1.238(0.8); 0.008(1.2); 0.000(29.7); −0.008(1.0)
Example 192: ¹H-NMR(400.0 MHz. DMSO): δ = 8.383(1.8); 8.373(1.7); 8.315(0.5); 7.222(3.0); 7.216(1.2); 7.207(3.0); 7.200(3.6); 7.191(1.5); 7.186(3.3); 7.179(0.4); 7.128(0.6);
7.121(3.6); 7.116(1.1); 7.104(1.5); 7.099(5.8); 7.089(4.4); 7.082(1.1); 7.076(2.4); 6.955(1.9); 3.902(1.9); 3.888(3.8); 3.877(16.0); 3.323(139.0); 2.948(0.5); 2.937(1.0); 2.928(1.5);
2.918(1.4); 2.909(1.0); 2.898(0.5); 2.680(0.4); 2.675(0.8); 2.671(1.1); 2.666(0.8); 2.662(0.4); 2.541(0.7); 2.524(2.9); 2.511(65.5); 2.506(130.2); 2.502(167.8); 2.497(118.2); 2.493(54.8);
2.337(0.4); 2.333(0.7); 2.328(1.0); 2.324(0.7); 2.071(0.7); 2.062(0.7); 2.055(0.9); 2.047(1.5); 2.039(0.9); 2.031(0.8); 2.023(0.7); 1.275(0.7); 1.261(1.5); 1.249(1.5); 1.237(1.6);
1.226(1.2); 1.219(1.3); 1.203(1.8); 1.200(1.6); 1.189(1.2); 1.184(1.7); 1.170(0.8); 0.008(1.6); 0.000(47.2); −0.009(1.5)
Example 193: ¹H-NMR(400.0 MHz. DMSO): δ = 8.426(1.6); 8.413(1.6); 8.315(0.5); 7.692(2.0); 7.673(2.2); 7.620(0.9); 7.601(2.0); 7.582(1.2); 7.420(1.2); 7.401(2.0); 7.382(0.9);
7.343(0.3); 7.305(2.1); 7.285(1.9); 7.209(0.7); 7.198(1.8); 7.075(0.4); 7.064(3.9); 6.929(1.9); 3.902(1.9); 3.886(6.7); 3.879(16.0); 3.323(145.7); 3.276(0.5); 3.263(0.9); 3.254(1.1);
3.242(1.1); 3.234(1.0); 3.221(0.4); 2.680(0.5); 2.675(0.9); 2.671(1.3); 2.666(0.9); 2.661(0.4); 2.541(0.7); 2.524(3.2); 2.511(77.6); 2.506(156.6); 2.502(204.7); 2.497(145.5); 2.493(68.3);
2.337(0.5); 2.333(0.9); 2.328(1.3); 2.324(0.9); 2.319(0.4); 2.255(0.9); 1.420(0.6); 1.406(1.5); 1.393(1.3); 1.383(2.3); 1.368(2.3); 1.348(1.4); 1.333(0.5); 0.008(2.1); 0.000(64.3); −0.009
(2.1)
Example 194: ¹H-NMR(400.0 MHz. DMSO): δ = 9.126(6.8); 9.113(6.7); 8.972(10.6); 8.967(10.6); 8.552(12.7); 8.547(12.1); 8.315(1.6); 7.598(15.7); 7.593(16.0); 7.446(0.4);
7.441(0.4); 7.382(7.0); 7.377(6.7); 7.361(8.6); 7.356(8.2); 7.208(0.4); 7.192(14.3); 7.171(11.5); 3.902(7.0); 3.508(0.3); 3.324(704.6); 3.186(1.5); 3.175(3.8); 3.165(4.5); 3.154(4.5);
3.145(3.7); 3.133(1.5); 2.675(3.2); 2.671(4.2); 2.666(3.0); 2.662(1.5); 2.623(0.4); 2.541(2.8); 2.524(12.8); 2.511(257.8); 2.506(499.8); 2.502(639.3); 2.497(451.9); 2.493(212.6);
2.333(2.9); 2.329(3.9); 2.324(2.8); 2.308(2.4); 2.299(2.7); 2.292(3.3); 2.284(5.2); 2.275(3.1); 2.268(2.9); 2.259(2.7); 1.422(2.6); 1.408(4.9); 1.396(4.7); 1.385(4.6); 1.372(3.2);
1.342(3.5); 1.326(5.7); 1.322(4.8); 1.311(3.8); 1.306(5.5); 1.292(2.6); 1.259(0.4); 1.235(0.5); 0.146(0.8); 0.008(7.6); 0.000(176.3); −0.009(6.0); −0.150(0.8)
Example 195: ¹H-NMR(400.0 MHz. DMSO): δ = 9.071(4.3); 9.060(4.2); 8.961(6.8); 8.957(6.9); 8.549(8.0); 8.544(7.7); 8.315(1.1); 7.344(11.8); 7.340(4.2); 7.328(5.3); 7.323(16.0);
7.317(2.1); 7.204(15.1); 7.183(11.1); 7.170(0.6); 7.148(0.4); 6.711(0.4); 6.690(0.3); 3.902(4.4); 3.322(336.7); 3.115(0.4); 3.102(0.5); 3.096(0.5); 3.085(0.5); 3.037(1.0); 3.025(2.3);
3.017(3.3); 3.006(3.2); 2.997(2.5); 2.986(1.0); 2.675(2.1); 2.671(2.8); 2.666(2.1); 2.541(1.7); 2.524(7.2); 2.510(177.5); 2.506(348.7); 2.502(446.9); 2.497(321.2); 2.493(154.4);
2.333(2.1); 2.328(2.8); 2.324(2.0); 2.092(1.6); 2.083(1.7); 2.076(2.1); 2.068(3.5); 2.060(2.1); 2.052(1.9); 2.044(1.8); 1.343(1.6); 1.329(3.2); 1.317(3.1); 1.305(3.1); 1.293(2.6);
1.288(2.8); 1.272(4.0); 1.268(3.5); 1.257(2.5); 1.253(3.8); 1.238(2.1); 1.194(1.0); 1.176(2.0); 1.158(1.1); 0.146(0.5); 0.008(4.5); 0.000(135.3); −0.008(5.3); −0.150(0.5)
Example 196: ¹H-NMR(400.0 MHz. DMSO): δ = 8.315(1.4); 7.456(0.3); 7.447(0.4); 7.332(2.4); 7.309(0.8); 7.288(1.9); 7.270(4.9); 7.252(7.5); 7.239(3.2); 7.234(7.1); 7.218(8.4);
7.201(3.2); 7.184(2.4); 7.180(2.0); 7.173(4.1); 7.166(6.6); 7.159(4.8); 7.150(1.5); 7.030(2.2); 7.021(2.4); 7.012(2.1); 7.007(1.8); 6.873(0.5); 6.553(0.6); 6.419(1.3); 6.328(1.0);
6.284(0.6); 6.194(2.1); 6.060(1.1); 4.716(0.5); 4.588(0.5); 4.583(0.5); 4.027(0.8); 3.902(13.4); 3.862(0.4); 3.836(0.8); 3.803(9.6); 3.777(0.9); 3.723(3.8); 3.648(16.0); 3.363(1.2);
3.324(660.6); 3.254(0.4); 2.832(0.9); 2.812(1.2); 2.785(0.6); 2.777(0.5); 2.710(15.0); 2.675(3.5); 2.670(5.4); 2.661(9.6); 2.541(1.5); 2.524(7.7); 2.510(214.6); 2.506(434.5);
2.501(568.9); 2.497(408.3); 2.492(195.2); 2.431(0.9); 2.415(0.6); 2.412(0.6); 2.389(0.5); 2.378(0.4); 2.367(0.4); 2.361(0.4); 2.337(1.5); 2.333(2.9); 2.328(3.8); 2.324(2.9); 2.281(0.5);
2.185(3.3); 1.971(1.1); 1.916(3.1); 1.856(3.6); 1.827(3.9); 1.746(1.5); 1.714(2.3); 1.707(2.2); 1.681(3.0); 1.581(1.1); 1.572(1.1); 1.541(1.9); 1.533(1.7); 1.509(2.5); 1.477(2.2);
1.447(0.9); 1.397(0.8); 1.366(1.4); 1.334(1.5); 1.301(0.8); 1.269(0.3); 1.258(0.4); 1.250(0.3); 1.235(0.5); 0.146(0.4); 0.008(3.1); 0.000(100.5); −0.009(3.7); −0.150(0.4)
Example 197: ¹H-NMR(400.0 MHz. DMSO): δ = 8.315(1.1); 7.412(1.9); 7.394(2.6); 7.329(0.4); 7.296(1.0); 7.285(2.0); 7.281(1.9); 7.266(5.0); 7.249(12.0); 7.244(9.0); 7.228(2.3);
7.215(0.4); 7.193(1.5); 7.188(1.8); 7.182(1.5); 7.179(1.4); 7.176(1.3); 7.172(2.3); 7.165(0.6); 7.162(0.6); 7.156(0.9); 7.151(0.4); 7.007(0.5); 6.988(0.6); 6.857(0.6); 4.775(0.4);
4.671(0.3); 4.658(0.4); 4.643(0.3); 3.914(0.9); 3.902(8.5); 3.879(2.2); 3.815(3.6); 3.737(16.0); 3.324(454.2); 2.825(3.1); 2.801(0.4); 2.680(0.7); 2.675(1.6); 2.671(2.2); 2.666(1.6);
2.662(0.7); 2.567(15.8); 2.541(1.1); 2.524(5.6); 2.511(137.2); 2.506(275.0); 2.502(358.7); 2.497(255.8); 2.492(120.7); 2.337(0.9); 2.333(1.8); 2.328(2.3); 2.324(1.7); 2.319(0.9);
2.109(5.5); 1.933(2.0); 1.858(1.8); 1.826(2.1); 1.796(0.5); 1.747(1.2); 1.715(1.3); 1.676(1.7); 1.618(1.2); 1.601(1.1); 1.543(0.8); 1.535(0.8); 1.512(1.6); 1.505(1.5); 1.481(1.5);
1.473(1.4); 1.448(0.8); 1.397(0.7); 1.366(1.1); 1.334(0.9); 0.008(2.7); 0.000(79.5); −0.009(2.7)
Example 198: ¹H-NMR(400.0 MHz. DMSO): δ = 8.737(4.1); 8.727(4.0); 8.315(0.5); 7.510(3.8); 7.502(1.4); 7.491(7.6); 7.486(5.1); 7.466(2.8); 7.461(3.3); 7.449(6.2); 7.444(9.0);
7.431(7.5); 7.427(10.6); 7.406(5.6); 7.403(5.2); 7.390(4.6); 7.387(4.4); 7.383(1.9); 7.370(1.6); 7.367(1.5); 7.342(11.8); 7.321(16.0); 7.299(0.5); 7.276(0.4); 7.201(15.2); 7.180(11.4);
7.133(0.6); 7.111(0.6); 6.697(0.5); 6.676(0.5); 3.902(1.8); 3.384(0.4); 3.375(0.4); 3.324(331.5); 3.026(1.1); 3.015(2.4); 3.007(3.4); 2.996(3.4); 2.987(2.5); 2.976(1.1); 2.675(1.2);
2.671(1.6); 2.666(1.3); 2.541(1.4); 2.510(111.7); 2.506(211.7); 2.502(267.9); 2.497(193.2); 2.493(95.2); 2.432(0.5); 2.333(1.2); 2.328(1.6); 2.324(1.2); 2.093(1.7); 2.084(1.8);
2.077(2.3); 2.069(3.7); 2.060(2.2); 2.053(2.1); 2.045(1.9); 1.308(1.8); 1.295(3.6); 1.283(3.3); 1.272(3.3); 1.259(2.8); 1.243(2.6); 1.228(4.2); 1.224(3.4); 1.213(2.6); 1.208(4.0);
1.194(1.7); 0.000(6.3)
Example 199: ¹H-NMR(400.0 MHz. DMSO): δ = 8.662(3.5); 8.651(3.4); 8.312(1.2); 7.882(4.8); 7.863(5.2); 7.458(2.1); 7.456(2.0); 7.440(5.0); 7.437(4.8); 7.421(3.4); 7.419(3.2);
7.344(13.1); 7.322(16.0); 7.202(12.4); 7.181(9.8); 7.169(4.3); 7.165(3.9); 7.150(2.2); 7.146(2.0); 7.133(0.4); 7.112(0.4); 6.699(0.3); 6.679(0.3); 3.901(2.1); 3.431(0.3); 3.407(0.5);
3.395(0.5); 3.370(1.2); 3.328(582.7); 3.265(0.5); 3.258(0.4); 2.991(0.9); 2.980(2.0); 2.971(2.8); 2.960(2.7); 2.952(2.1); 2.940(0.8); 2.676(1.3); 2.671(1.7); 2.667(1.2); 2.549(0.8);
2.541(1.5); 2.511(118.4); 2.507(221.6); 2.502(279.4); 2.498(200.6); 2.493(97.9); 2.443(0.5); 2.333(1.3); 2.329(1.6); 2.324(1.2); 2.130(1.4); 2.121(1.5); 2.114(1.9); 2.106(3.0);
2.098(1.8); 2.090(1.7); 2.082(1.5); 1.326(1.5); 1.312(2.8); 1.300(2.8); 1.289(2.6); 1.277(1.8); 1.255(2.1); 1.240(3.4); 1.235(2.9); 1.225(2.2); 1.220(3.0); 1.205(1.3); 0.000(5.4)
Example 200: ¹H-NMR(400.0 MHz. DMSO): δ = 9.040(3.3); 9.030(3.3); 7.560(1.1); 7.543(2.4); 7.539(2.0); 7.526(1.6); 7.522(4.3); 7.518(1.5); 7.505(2.0); 7.501(2.5); 7.484(1.1);
7.349(1.3); 7.343(11.6); 7.338(3.8); 7.326(4.4); 7.321(16.0); 7.315(1.8); 7.213(2.0); 7.206(15.4); 7.201(5.3); 7.194(8.0); 7.190(4.5); 7.185(11.8); 7.175(9.5); 7.154(6.4); 7.146(1.1);
7.134(0.7); 6.756(0.6); 6.735(0.5); 5.756(1.5); 3.325(48.1); 3.062(0.9); 3.050(1.9); 3.042(2.4); 3.035(1.8); 3.031(2.4); 3.024(2.0); 3.011(1.0); 2.675(0.3); 2.671(0.5); 2.666(0.3);
2.524(1.2); 2.519(2.0); 2.511(25.0); 2.506(50.1); 2.502(66.0); 2.497(47.2); 2.493(22.0); 2.333(0.3); 2.329(0.4); 2.063(1.6); 2.054(1.5); 2.046(2.1); 2.039(2.6); 2.031(1.8); 2.023(1.8);
2.015(1.7); 1.336(1.0); 1.280(0.9); 1.266(3.7); 1.258(4.0); 1.254(3.6); 1.250(2.3); 1.244(5.8); 1.241(5.8); 1.231(3.2); 1.223(3.7); 1.208(0.8); 0.008(0.9); 0.000(26.7); −0.009(0.8)
Example 201: ¹H-NMR(400.0 MHz. DMSO): δ = 8.796(4.3); 8.786(4.3); 8.315(0.7); 7.793(4.6); 7.774(6.0); 7.743(2.0); 7.726(5.2); 7.707(3.8); 7.667(3.6); 7.648(4.6); 7.629(1.6);
7.590(0.3); 7.550(5.5); 7.531(4.6); 7.343(12.1); 7.322(16.0); 7.195(15.2); 7.174(11.5); 7.138(0.4); 7.117(0.4); 3.902(1.8); 3.397(0.3); 3.360(1.0); 3.325(359.0); 3.284(0.5); 3.262(0.3);
2.991(1.0); 2.980(2.4); 2.971(3.3); 2.961(3.3); 2.952(2.4); 2.941(1.1); 2.675(1.3); 2.671(1.8); 2.666(1.3); 2.587(0.3); 2.576(0.4); 2.541(1.6); 2.510(118.8); 2.506(223.9); 2.502(283.5);
2.497(205.6); 2.443(0.5); 2.333(1.3); 2.328(1.7); 2.324(1.2); 2.060(1.7); 2.051(1.9); 2.043(2.4); 2.036(3.6); 2.028(2.2); 2.021(2.0); 2.012(1.9); 1.298(0.3); 1.283(1.4); 1.269(3.8);
1.257(3.7); 1.247(4.7); 1.233(7.1); 1.215(3.8); 1.201(1.3); 0.000(8.0)
Example 202: ¹H-NMR(400.0 MHz. DMSO): δ = 8.568(4.0); 8.558(3.9); 8.315(0.6); 7.716(7.4); 7.703(7.7); 7.341(8.9); 7.320(12.0); 7.217(8.7); 7.205(16.0); 7.185(7.0); 3.902(2.0);
3.395(0.3); 3.323(299.4); 2.991(0.8); 2.981(1.9); 2.971(2.6); 2.961(2.7); 2.952(2.0); 2.675(1.5); 2.671(1.9); 2.666(1.4); 2.565(0.6); 2.560(0.6); 2.541(1.6); 2.510(130.3); 2.506(244.9);
2.502(309.8); 2.497(224.7); 2.493(111.0); 2.333(1.4); 2.328(1.8); 2.324(1.4); 2.117(1.5); 2.108(1.6); 2.100(2.0); 2.093(3.1); 2.085(1.9); 2.077(1.8); 2.068(1.6); 1.373(1.8); 1.360(3.0);
1.348(2.9); 1.337(2.8); 1.324(1.7); 1.298(0.4); 1.257(2.4); 1.242(3.9); 1.238(3.3); 1.222(4.2); 1.208(1.6); 1.187(0.4); 0.000(9.3)
Example 203: ¹H-NMR(400.0 MHz. DMSO): δ = 8.448(4.2); 8.438(4.1); 7.834(9.1); 7.821(9.6); 7.336(11.8); 7.315(16.0); 7.206(13.4); 7.185(9.9); 7.147(9.1); 7.134(8.7); 6.574(1.7);
5.757(1.3); 5.756(1.2); 3.324(51.8); 2.988(1.1); 2.978(2.5); 2.968(3.5); 2.958(3.5); 2.949(2.5); 2.938(1.1); 2.675(0.5); 2.671(0.7); 2.666(0.5); 2.523(2.6); 2.510(38.5); 2.506(73.3);
2.502(95.3); 2.497(70.0); 2.493(34.3); 2.333(0.5); 2.328(0.6); 2.324(0.5); 2.129(1.8); 2.121(2.0); 2.113(2.4); 2.105(3.9); 2.097(2.4); 2.090(2.2); 2.081(2.0); 1.393(2.0); 1.379(3.6);
1.368(3.6); 1.356(3.4); 1.343(2.4); 1.336(1.8); 1.261(2.8); 1.246(5.1); 1.242(3.8); 1.231(3.2); 1.226(4.6); 1.212(2.1); 1.187(0.6); 0.007(0.7); 0.000(14.6); −0.001(14.0); −0.008(0.5)
Example 204: ¹H-NMR(400.0 MHz. DMSO): δ = 8.726(3.6); 8.715(3.6); 7.898(13.0); 7.893(12.9); 7.340(1.3); 7.333(11.6); 7.329(3.8); 7.317(4.4); 7.312(16.0); 7.306(1.8); 7.201(1.9);
7.195(14.2); 7.190(4.0); 7.178(3.6); 7.173(10.5); 7.167(1.1); 6.854(13.7); 6.850(13.4); 5.757(1.2); 3.326(43.6); 2.939(1.0); 2.927(2.1); 2.919(3.1); 2.908(3.0); 2.899(2.2); 2.888(1.0);
2.672(0.4); 2.525(1.2); 2.520(1.9); 2.512(22.0); 2.507(44.0); 2.503(57.9); 2.498(41.4); 2.493(19.2); 2.329(0.4); 2.106(1.4); 2.098(1.5); 2.091(1.9); 2.082(3.2); 2.074(1.8); 2.067(1.7);
2.059(1.6); 1.422(1.9); 1.408(2.9); 1.398(2.7); 1.396(2.8); 1.386(2.7); 1.372(1.9); 1.336(1.0); 1.259(0.4); 1.249(1.4); 1.242(2.3); 1.227(3.5); 1.223(2.8); 1.212(2.4); 1.207(3.4);
1.193(1.8); 0.008(0.4); 0.000(12.7); −0.009(0.4)
Example 205: ¹H-NMR(400.0 MHz. DMSO): δ = 8.688(3.7); 8.676(3.7); 8.315(0.6); 7.611(2.4); 7.607(2.8); 7.594(14.5); 7.588(16.0); 7.574(2.8); 7.569(3.0); 7.558(1.4); 7.553(1.3);
7.544(1.6); 7.539(3.0); 7.525(2.6); 7.519(3.1); 7.514(1.8); 7.505(1.7); 7.501(1.4); 7.381(4.7); 7.376(4.5); 7.360(5.9); 7.355(5.6); 7.310(3.7); 7.294(4.9); 7.288(4.2); 7.285(4.5);
7.283(4.5); 7.277(7.5); 7.262(3.7); 7.259(4.1); 7.210(9.7); 7.189(7.8); 3.902(1.8); 3.385(0.5); 3.369(0.6); 3.326(390.1); 3.290(0.8); 3.273(0.4); 3.261(0.4); 3.221(1.2); 3.209(2.6);
3.200(3.2); 3.189(3.3); 3.180(2.6); 3.168(1.1); 2.675(1.2); 2.671(1.6); 2.666(1.2); 2.541(1.3); 2.511(110.0); 2.506(208.4); 2.502(264.1); 2.497(189.8); 2.493(92.0); 2.333(1.3);
2.329(1.6); 2.324(1.4); 2.317(2.0); 2.308(2.0); 2.301(2.4); 2.293(3.8); 2.284(2.4); 2.277(2.2); 2.268(1.9); 1.425(2.0); 1.412(3.7); 1.400(3.5); 1.388(3.5); 1.375(2.2); 1.272(2.4);
1.257(4.2); 1.253(3.4); 1.242(2.9); 1.237(4.0); 1.223(2.0); 0.000(6.5)
Example 206: ¹H-NMR(400.0 MHz. DMSO): δ = 8.798(6.4); 8.785(6.4); 8.315(0.9); 7.595(15.1); 7.590(15.4); 7.515(6.0); 7.507(2.3); 7.496(11.3); 7.492(8.1); 7.472(4.4); 7.467(5.2);
7.455(9.5); 7.450(13.7); 7.437(11.3); 7.433(16.0); 7.413(8.6); 7.409(8.0); 7.396(6.8); 7.394(6.8); 7.389(3.3); 7.381(7.4); 7.376(8.5); 7.360(8.4); 7.355(8.0); 7.306(0.3); 7.298(0.4);
7.203(13.9); 7.182(11.1); 3.902(3.2); 3.409(0.5); 3.397(0.6); 3.325(612.4); 3.262(0.6); 3.245(0.3); 3.222(0.4); 3.199(0.5); 3.190(1.7); 3.178(3.9); 3.169(5.1); 3.158(4.9); 3.149(3.9);
3.137(1.7); 2.675(2.1); 2.671(2.9); 2.666(2.1); 2.541(3.3); 2.506(362.5); 2.502(457.7); 2.497(334.4); 2.406(0.4); 2.380(0.5); 2.329(2.8); 2.324(2.2); 2.308(2.7); 2.299(3.1); 2.292(3.6);
2.284(5.6); 2.275(3.5); 2.268(3.2); 2.259(2.9); 1.383(2.8); 1.370(5.3); 1.358(5.2); 1.346(5.1); 1.333(3.3); 1.318(0.3); 1.285(3.6); 1.269(6.0); 1.265(5.0); 1.254(4.2); 1.250(5.6);
1.235(2.9); 0.000(8.8)
Example 207: ¹H-NMR(400.0 MHz. DMSO): δ = 8.783(5.2); 8.770(5.2); 8.316(1.2); 7.669(7.1); 7.667(7.4); 7.649(8.2); 7.647(8.3); 7.595(14.1); 7.590(14.5); 7.584(0.6); 7.460(2.0);
7.457(2.3); 7.441(6.5); 7.438(6.0); 7.424(10.7); 7.421(16.0); 7.415(13.3); 7.407(0.9); 7.402(4.1); 7.396(2.0); 7.385(7.6); 7.381(7.7); 7.379(6.9); 7.376(6.6); 7.368(4.9); 7.365(6.3);
7.360(11.4); 7.355(7.9); 7.349(4.0); 7.343(3.1); 7.202(13.5); 7.181(10.9); 3.323(209.7); 3.300(0.7); 3.175(1.4); 3.163(3.1); 3.154(3.9); 3.146(2.7); 3.142(4.0); 3.134(3.3); 3.122(1.4);
2.679(0.6); 2.675(1.3); 2.670(1.7); 2.666(1.3); 2.661(0.6); 2.524(5.0); 2.519(8.0); 2.511(91.3); 2.506(182.2); 2.501(240.7); 2.497(172.3); 2.492(80.4); 2.337(0.6); 2.333(1.3); 2.328(1.8);
2.321(2.6); 2.312(2.3); 2.305(2.9); 2.297(4.7); 2.288(2.8); 2.281(2.6); 2.272(2.4); 1.388(2.6); 1.374(4.4); 1.362(4.3); 1.351(4.2); 1.338(3.4); 1.298(0.4); 1.285(3.2); 1.269(4.8);
1.265(4.2); 1.255(3.4); 1.249(5.6); 1.235(2.5); 0.008(1.5); 0.000(44.6); −0.009(1.3)
Example 208: ¹H-NMR(400.0 MHz. DMSO): δ = 8.729(7.6); 8.717(7.7); 8.316(4.0); 7.889(10.7); 7.870(11.4); 7.804(0.3); 7.593(15.2); 7.588(15.8); 7.465(4.8); 7.463(4.3);
7.446(11.0); 7.444(10.1); 7.428(7.4); 7.425(6.7); 7.381(7.5); 7.376(7.2); 7.360(9.8); 7.354(15.3); 7.349(11.3); 7.334(8.0); 7.330(7.3); 7.218(0.4); 7.203(16.0); 7.194(6.0); 7.190(6.0);
7.182(13.4); 7.175(9.1); 7.171(8.3); 7.156(4.7); 7.151(4.3); 3.323(157.0); 3.299(2.3); 3.155(1.6); 3.143(4.0); 3.134(5.3); 3.123(5.3); 3.113(4.3); 3.102(1.8); 2.675(1.1); 2.670(1.5);
2.666(1.1); 2.524(4.3); 2.506(172.4); 2.501(221.6); 2.497(162.3); 2.351(2.9); 2.342(3.1); 2.335(4.3); 2.327(7.4); 2.319(4.1); 2.312(3.4); 2.303(3.1); 1.404(3.0); 1.391(5.7); 1.379(5.7);
1.368(5.6); 1.354(3.4); 1.336(0.6); 1.285(3.7); 1.269(6.4); 1.265(5.2); 1.254(4.6); 1.250(6.6); 1.235(3.0); 0.008(1.2); 0.000(31.0); −0.009(1.2)
Example 209: ¹H-NMR(400.0 MHz. DMSO): δ = 9.096(3.6); 9.083(3.5); 8.315(0.8); 7.601(10.2); 7.596(10.3); 7.565(1.0); 7.549(2.3); 7.544(2.0); 7.528(4.1); 7.511(2.1); 7.507(2.5);
7.490(1.1); 7.456(0.4); 7.451(0.4); 7.382(4.4); 7.376(4.0); 7.361(5.3); 7.355(5.0); 7.199(15.1); 7.179(16.0); 7.159(5.9); 7.151(1.2); 3.902(1.9); 3.322(208.8); 3.208(0.9); 3.196(2.2);
3.187(2.7); 3.180(2.5); 3.176(2.8); 3.167(2.4); 3.155(1.0); 2.675(1.4); 2.671(1.9); 2.666(1.3); 2.541(1.3); 2.511(127.8); 2.506(243.0); 2.502(306.5); 2.497(216.9); 2.493(102.7);
2.333(1.4); 2.328(1.9); 2.324(1.3); 2.319(0.7); 2.268(1.6); 2.259(1.7); 2.252(2.2); 2.244(3.0); 2.236(2.0); 2.228(1.9); 2.219(1.7); 1.348(1.3); 1.334(3.5); 1.321(3.4); 1.316(3.6);
1.310(3.8); 1.298(6.6); 1.280(3.4); 1.266(1.2); 1.259(0.9); 0.008(0.5); 0.000(12.2); −0.009(0.4)
Example 210: ¹H-NMR(400.0 MHz. DMSO): δ = 8.858(7.2); 8.846(7.0); 8.315(0.9); 7.798(7.6); 7.779(10.0); 7.750(3.3); 7.732(8.4); 7.713(6.2); 7.673(5.9); 7.653(7.5); 7.635(2.8);
7.595(15.7); 7.589(16.0); 7.557(9.1); 7.539(7.6); 7.458(0.4); 7.380(7.3); 7.374(6.8); 7.359(8.9); 7.353(8.4); 7.194(14.6); 7.173(11.8); 3.902(2.6); 3.397(0.3); 3.322(330.3); 3.256(0.5);
3.155(1.6); 3.143(4.1); 3.134(5.3); 3.123(5.4); 3.114(4.2); 3.102(1.7); 2.671(3.0); 2.666(2.3); 2.579(0.4); 2.541(2.6); 2.506(401.5); 2.502(497.9); 2.497(364.3); 2.408(0.7); 2.385(0.6);
2.328(3.0); 2.282(2.9); 2.273(3.2); 2.266(3.9); 2.257(6.0); 2.249(3.8); 2.242(3.5); 2.233(3.1); 1.364(2.8); 1.350(5.7); 1.338(5.5); 1.327(5.5); 1.314(3.7); 1.292(3.9); 1.276(6.6);
1.257(6.3); 1.242(2.8); 0.000(16.2)
Example 211: ¹H-NMR(400.0 MHz. DMSO): δ = 8.961(5.8); 8.949(5.7); 8.488(8.3); 8.483(8.7); 8.476(8.8); 8.471(8.3); 8.315(1.1); 7.940(8.2); 7.936(8.2); 7.922(9.4); 7.917(8.6);
7.601(15.8); 7.595(16.0); 7.517(8.7); 7.505(8.6); 7.499(8.1); 7.486(8.0); 7.472(0.4); 7.395(0.5); 7.384(6.5); 7.379(6.2); 7.363(8.1); 7.358(7.7); 7.211(13.3); 7.190(10.7); 7.179(0.5);
3.902(6.3); 3.434(0.4); 3.396(0.3); 3.325(469.5); 3.292(1.1); 3.268(0.6); 3.264(0.6); 3.187(1.5); 3.175(3.7); 3.167(5.4); 3.155(4.5); 3.146(3.7); 3.134(1.5); 2.675(1.9); 2.671(2.6);
2.666(1.8); 2.541(1.9); 2.524(7.7); 2.511(176.1); 2.506(340.5); 2.502(434.0); 2.497(310.1); 2.493(148.7); 2.443(0.8); 2.440(0.8); 2.333(2.0); 2.329(2.7); 2.321(3.2); 2.312(2.7);
2.305(3.2); 2.296(5.0); 2.288(3.1); 2.281(2.9); 2.272(2.6); 1.380(2.4); 1.366(4.7); 1.354(4.6); 1.343(4.5); 1.330(3.3); 1.313(3.7); 1.297(5.8); 1.294(4.9); 1.282(3.6); 1.278(5.2);
1.263(2.4); 1.236(0.6); 0.000(14.8); −0.009(0.5)
Example 212: ¹H-NMR(400.0 MHz. DMSO): δ = 8.615(4.5); 8.603(4.5); 8.316(0.8); 7.722(11.6); 7.710(12.2); 7.595(10.2); 7.590(10.6); 7.381(4.6); 7.376(4.4); 7.360(5.8); 7.355(5.6);
7.222(13.1); 7.209(16.0); 7.186(5.1); 3.323(136.1); 3.299(0.4); 3.174(0.9); 3.162(2.3); 3.153(3.0); 3.142(3.0); 3.133(2.4); 3.121(1.0); 2.680(0.5); 2.675(1.0); 2.671(1.4); 2.666(1.0);
2.662(0.5); 2.524(4.8); 2.511(75.6); 2.506(148.5); 2.502(195.3); 2.497(141.3); 2.493(67.4); 2.341(1.9); 2.332(2.8); 2.325(3.2); 2.317(4.0); 2.308(2.3); 2.301(2.2); 2.292(2.0); 1.989(0.9);
1.452(2.1); 1.439(3.7); 1.427(3.5); 1.415(3.4); 1.402(2.2); 1.336(1.2); 1.283(2.2); 1.268(3.8); 1.263(3.1); 1.252(2.9); 1.249(4.6); 1.233(2.0); 1.187(0.5); 1.175(0.5); 0.008(1.4);
0.000(36.3); −0.009(1.2)
Example 213: ¹H-NMR(400.0 MHz. DMSO): δ = 8.498(5.2); 8.487(5.3); 7.842(15.4); 7.829(16.0); 7.593(13.3); 7.587(14.0); 7.378(6.2); 7.372(6.0); 7.357(7.8); 7.351(7.6); 7.211(9.7);
7.190(7.7); 7.154(15.6); 7.141(15.1); 6.575(0.6); 5.757(1.2); 3.326(88.0); 3.184(1.3); 3.172(3.3); 3.163(4.2); 3.152(4.3); 3.143(3.4); 3.131(1.4); 2.676(0.5); 2.672(0.7); 2.667(0.5);
2.525(2.0); 2.520(3.1); 2.512(38.4); 2.507(77.7); 2.503(103.4); 2.498(75.6); 2.494(36.6); 2.342(2.4); 2.333(3.0); 2.326(3.4); 2.318(5.2); 2.309(3.1); 2.303(2.9); 2.293(2.7); 2.080(0.5);
2.036(0.4); 1.476(2.8); 1.463(4.8); 1.451(4.7); 1.439(4.6); 1.426(3.0); 1.336(2.1); 1.277(3.2); 1.261(5.3); 1.257(4.5); 1.249(3.9); 1.247(4.3); 1.242(5.3); 1.227(2.8); 1.188(0.9);
0.008(0.7); 0.000(20.0); −0.009(0.6)
Example 214: ¹H-NMR(400.0 MHz. DMSO): δ = 8.766(4.9); 8.754(4.9); 8.315(1.0); 7.907(14.8); 7.902(14.5); 7.583(13.3); 7.578(13.6); 7.375(5.6); 7.370(5.2); 7.354(7.0); 7.349(6.7);
7.199(11.1); 7.178(8.8); 6.861(16.0); 6.857(15.4); 3.902(3.7); 3.379(0.4); 3.374(0.5); 3.324(432.4); 3.282(0.4); 3.261(0.4); 3.161(1.2); 3.148(2.9); 3.139(3.6); 3.128(3.7); 3.119(3.0);
3.106(1.3); 2.680(0.9); 2.676(1.8); 2.671(2.4); 2.667(1.7); 2.662(0.9); 2.589(0.3); 2.541(1.9); 2.524(7.0); 2.511(156.7); 2.507(309.1); 2.502(398.0); 2.497(283.1); 2.493(134.4);
2.449(0.5); 2.439(0.5); 2.333(2.8); 2.328(4.1); 2.320(3.4); 2.312(4.3); 2.303(2.6); 2.297(2.4); 2.287(2.2); 1.504(2.3); 1.491(4.2); 1.479(3.8); 1.468(4.0); 1.454(2.4); 1.299(1.2);
1.259(1.8); 1.251(2.8); 1.236(4.7); 1.231(3.7); 1.221(3.4); 1.216(4.3); 1.201(2.3); 0.008(0.4); 0.000(13.4); −0.008(0.4)
Example 215: ¹H-NMR(400.0 MHz. DMSO): δ = 9.032(0.4); 8.955(0.5); 8.929(0.6); 8.567(1.2); 8.563(1.3); 8.555(1.5); 8.552(1.5); 8.535(1.1); 8.531(1.0); 8.512(6.5); 8.490(6.6);
8.462(0.5); 8.432(13.9); 8.429(14.1); 8.420(14.7); 8.417(14.0); 8.315(1.8); 8.039(1.3); 8.036(1.2); 8.019(1.4); 8.015(1.2); 8.009(0.7); 7.992(0.5); 7.988(0.6); 7.888(14.0); 7.884(13.9);
7.867(15.9); 7.864(14.6); 7.838(0.3); 7.834(0.3); 7.774(8.9); 7.753(10.9); 7.723(0.4); 7.704(0.5); 7.686(0.4); 7.670(0.6); 7.639(13.8); 7.619(16.0); 7.605(5.8); 7.543(1.2); 7.531(1.2);
7.522(1.3); 7.510(1.2); 7.506(0.6); 7.468(0.4); 7.453(15.2); 7.441(14.3); 7.432(14.1); 7.421(13.9); 7.413(6.9); 7.393(10.4); 7.375(4.7); 4.889(1.2); 4.870(3.1); 4.854(4.9); 4.837(3.7);
4.819(1.4); 4.551(0.4); 4.539(0.4); 4.202(1.8); 4.183(4.8); 4.164(4.8); 4.144(1.8); 3.902(4.9); 3.323(469.7); 3.295(1.3); 3.282(0.7); 3.251(0.4); 2.675(3.2); 2.671(4.3); 2.666(3.1);
2.632(0.6); 2.609(0.6); 2.548(1.8); 2.541(2.6); 2.524(13.5); 2.511(282.8); 2.506(548.7); 2.502(699.8); 2.497(499.0); 2.493(241.3); 2.476(8.3); 2.469(6.8); 2.446(4.3); 2.429(2.3);
2.352(0.5); 2.333(3.5); 2.329(4.6); 2.324(3.2); 2.300(0.7); 2.278(1.4); 2.252(3.7); 2.246(2.8); 2.237(4.0); 2.231(4.8); 2.225(3.6); 2.216(3.1); 2.210(4.3); 2.189(2.1); 2.184(2.0);
2.176(3.2); 2.154(4.0); 2.138(4.7); 2.126(4.2); 2.115(2.6); 2.103(0.9); 2.092(1.2); 2.071(0.4); 2.043(0.4); 2.017(0.4); 1.977(0.4); 1.952(0.7); 1.929(1.0); 1.906(1.0); 1.892(0.8);
1.859(0.5); 1.716(0.5); 1.693(0.7); 1.669(0.9); 1.656(0.7); 1.649(0.7); 1.538(0.3); 1.519(0.3); 1.494(0.6); 1.482(0.7); 1.458(0.8); 1.444(0.8); 1.411(0.8); 1.381(0.6); 1.336(1.2);
1.327(0.4); 1.298(0.9); 1.274(0.7); 1.259(1.3); 1.249(1.4); 1.235(3.1); 1.225(1.8); 1.200(0.4); 0.853(0.5); 0.833(0.3); 0.008(1.1); 0.000(29.4); −0.009(0.9)
Example 216: ¹H-NMR(400.0 MHz. DMSO): δ = 8.838(0.4); 8.816(0.4); 8.790(0.3); 8.762(0.4); 8.458(7.2); 8.436(7.2); 8.315(1.4); 7.883(0.6); 7.861(0.8); 7.763(7.3); 7.743(13.5);
7.708(7.0); 7.690(10.2); 7.671(15.6); 7.651(13.8); 7.632(1.0); 7.609(0.8); 7.559(0.6); 7.543(10.6); 7.540(8.5); 7.525(12.5); 7.520(12.4); 7.503(0.6); 7.462(6.3); 7.443(10.3); 7.425(4.8);
7.397(0.7); 7.384(0.5); 7.380(0.5); 7.360(1.0); 7.352(0.7); 7.338(0.8); 7.329(0.9); 7.325(0.9); 7.313(3.2); 7.309(4.9); 7.294(10.7); 7.291(11.8); 7.281(12.1); 7.278(15.1); 7.274(16.0);
7.262(10.9); 7.257(9.8); 7.243(3.8); 7.238(2.9); 6.716(11.1); 6.710(11.4); 6.698(8.2); 6.693(10.4); 4.839(1.7); 4.826(3.7); 4.819(4.0); 4.807(4.0); 4.798(3.8); 4.775(1.4); 4.191(1.8);
4.173(4.9); 4.153(4.6); 4.132(1.7); 3.902(5.6); 3.389(0.4); 3.375(0.4); 3.325(781.2); 3.256(0.7); 3.241(0.6); 3.202(0.5); 3.193(0.4); 3.170(1.4); 2.675(2.8); 2.671(3.8); 2.666(2.7);
2.600(0.4); 2.571(0.4); 2.541(2.6); 2.510(265.6); 2.506(510.1); 2.502(655.4); 2.497(476.3); 2.493(237.8); 2.464(7.1); 2.453(6.1); 2.435(2.6); 2.411(0.9); 2.398(0.9); 2.333(3.3);
2.328(4.2); 2.324(3.2); 2.257(1.2); 2.234(3.1); 2.214(4.4); 2.207(5.0); 2.202(4.0); 2.188(3.6); 2.174(1.4); 2.164(2.1); 2.128(3.0); 2.115(2.2); 2.104(4.1); 2.095(5.1); 2.083(4.6);
2.059(2.1); 2.048(1.0); 2.037(0.9); 2.014(0.6); 1.980(0.6); 1.956(0.7); 1.930(0.6); 1.878(0.7); 1.860(0.6); 1.832(0.4); 1.755(0.3); 1.731(0.4); 1.706(0.5); 1.673(0.5); 1.653(0.5);
1.620(0.6); 1.571(0.4); 1.449(0.6); 1.436(0.6); 1.413(0.6); 1.384(0.5); 1.337(0.4); 1.317(0.4); 1.259(0.9); 1.237(0.8); 0.809(0.3); 0.000(18.5); −0.009(0.7)
Example 217: ¹H-NMR(600.1 MHz. DMSO): δ = 8.903(0.4); 8.888(0.4); 8.486(6.1); 8.472(6.1); 7.870(0.5); 7.857(0.6); 7.740(6.2); 7.727(9.8); 7.717(0.7); 7.700(1.2); 7.697(1.3);
7.690(5.4); 7.677(8.1); 7.660(9.3); 7.647(9.2); 7.554(15.7); 7.551(16.0); 7.522(0.6); 7.519(0.5); 7.509(0.8); 7.506(0.6); 7.485(0.6); 7.482(0.6); 7.471(0.7); 7.468(0.7); 7.449(4.6);
7.436(8.1); 7.423(3.9); 7.385(8.2); 7.382(7.9); 7.372(8.8); 7.368(9.4); 7.355(1.0); 6.857(14.4); 6.843(13.9); 4.839(0.9); 4.829(2.5); 4.817(3.9); 4.805(3.1); 4.792(1.0); 4.766(0.4);
4.180(1.4); 4.167(3.8); 4.155(3.8); 4.142(1.5); 3.903(0.4); 3.331(257.5); 2.614(1.1); 2.520(4.2); 2.517(4.7); 2.505(144.0); 2.502(183.9); 2.500(139.7); 2.484(5.8); 2.476(7.5); 2.460(4.0);
2.445(2.7); 2.433(1.2); 2.386(1.1); 2.246(0.9); 2.228(2.6); 2.225(2.3); 2.218(3.1); 2.214(3.5); 2.210(2.7); 2.204(2.3); 2.200(2.9); 2.185(1.2); 2.153(0.3); 2.136(0.4); 2.115(1.6);
2.100(2.5); 2.096(2.3); 2.088(3.3); 2.080(3.2); 2.074(2.0); 2.065(0.7); 2.058(0.8); 2.048(0.5); 2.032(0.5); 2.016(0.3); 1.938(0.4); 1.910(0.4); 1.864(0.3); 1.730(0.4); 1.627(0.3);
1.623(0.3); 1.607(0.3); 1.447(0.4); 1.258(0.6); 1.235(1.2); 0.854(0.3); 0.000(2.7)
Example 218: ¹H-NMR(400.0 MHz. DMSO): δ = 12.545(1.7); 9.978(3.7); 9.966(3.7); 8.336(5.4); 8.331(5.7); 8.318(5.9); 8.313(5.6); 7.731(2.0); 7.725(2.2); 7.715(3.9); 7.710(3.8);
7.699(2.1); 7.694(1.8); 7.293(3.8); 7.274(10.5); 7.256(7.6); 7.188(3.7); 7.185(3.3); 7.169(16.0); 7.166(12.5); 7.148(11.3); 6.504(3.0); 6.486(5.2); 6.470(2.8); 3.902(3.7); 3.360(0.5);
3.324(167.1); 3.047(1.1); 3.033(2.1); 3.026(2.4); 3.021(2.7); 3.015(2.2); 3.008(2.1); 2.995(1.1); 2.676(1.1); 2.671(1.4); 2.667(1.0); 2.541(1.1); 2.524(4.4); 2.511(92.6); 2.507(179.4);
2.502(228.6); 2.498(161.7); 2.493(76.1); 2.333(1.0); 2.329(1.4); 2.324(1.0); 2.092(1.7); 2.084(1.8); 2.076(2.0); 2.070(2.7); 2.063(2.4); 2.053(1.9); 2.045(1.8); 1.296(0.7); 1.282(5.1);
1.280(5.3); 1.265(10.8); 1.256(3.9); 1.247(4.0); 1.243(4.7); 1.229(0.6); 0.008(0.4); 0.000(9.6)
Example 219: ¹H-NMR(400.0 MHz. DMSO): δ = 8.412(0.9); 8.402(0.9); 8.312(0.5); 8.295(1.4); 8.290(1.5); 8.283(1.5); 8.278(1.5); 8.058(1.4); 8.053(1.4); 8.040(1.5); 8.035(1.4);
7.298(1.0); 7.279(2.7); 7.261(2.0); 7.191(1.0); 7.188(0.9); 7.171(3.5); 7.168(3.3); 7.150(2.6); 7.120(1.5); 7.107(1.5); 7.101(1.5); 7.089(1.4); 3.961(16.0); 3.900(0.5); 3.331(59.0);
3.327(62.7); 3.044(0.6); 3.036(0.9); 3.025(0.9); 3.016(0.7); 2.672(0.4); 2.525(1.2); 2.511(25.8); 2.507(50.7); 2.503(65.5); 2.498(47.9); 2.494(23.9); 2.329(0.4); 2.112(0.4); 2.103(0.5);
2.096(0.6); 2.088(1.0); 2.079(0.6); 2.073(0.5); 2.064(0.5); 1.361(0.5); 1.347(0.9); 1.336(0.9); 1.324(0.8); 1.311(0.6); 1.249(0.7); 1.234(1.0); 1.230(0.8); 1.220(0.7); 1.215(1.0);
1.200(0.5); 0.000(2.2)
Example 220: ¹H-NMR(400.0 MHz. DMSO): δ = 8.367(2.2); 8.306(0.9); 8.304(1.0); 8.300(1.2); 8.298(1.3); 8.274(3.0); 8.270(3.2); 8.263(3.0); 8.262(3.1); 8.258(3.1); 8.070(2.5);
8.067(2.5); 8.063(1.6); 8.051(2.6); 8.049(2.5); 7.299(2.7); 7.281(7.2); 7.262(5.5); 7.191(2.4); 7.173(4.6); 7.168(8.3); 7.165(7.9); 7.147(6.3); 7.103(2.7); 7.101(2.7); 7.091(2.8);
7.089(2.8); 7.084(2.8); 7.083(2.7); 7.072(2.5); 7.071(2.5); 4.453(1.7); 4.437(5.3); 4.419(5.5); 4.402(1.9); 3.896(0.6); 3.893(0.5); 3.391(0.5); 3.337(224.5); 3.333(204.3); 3.328(210.1);
3.069(0.8); 3.057(1.9); 3.049(2.6); 3.038(2.6); 3.029(2.0); 3.018(0.9); 2.678(0.8); 2.674(1.2); 2.669(0.8); 2.544(0.6); 2.527(3.3); 2.514(76.6); 2.509(151.4); 2.505(195.6); 2.500(139.7);
2.496(66.7); 2.341(0.4); 2.336(0.8); 2.332(1.1); 2.327(0.8); 2.322(0.4); 2.102(1.3); 2.094(1.4); 2.087(1.7); 2.079(2.8); 2.071(1.6); 2.063(1.5); 2.055(1.4); 1.402(7.7); 1.384(16.0);
1.367(7.6); 1.328(1.4); 1.315(2.7); 1.303(2.6); 1.291(2.5); 1.279(2.1); 1.273(2.2); 1.258(3.1); 1.254(2.7); 1.243(1.8); 1.238(3.1); 1.224(1.3); 1.194(0.3); 0.000(3.9)
Example 221: ¹H-NMR(400.0 MHz. DMSO): δ = 9.028(4.8); 9.018(4.7); 8.925(7.2); 8.913(7.0); 8.839(13.7); 8.311(2.0); 7.839(7.8); 7.826(7.5); 7.305(5.5); 7.287(14.6); 7.268(10.9);
7.200(5.1); 7.197(3.7); 7.182(7.6); 7.177(3.1); 7.167(14.3); 7.164(16.0); 7.147(11.0); 7.090(0.4); 3.899(2.0); 3.328(314.2); 3.269(0.6); 3.032(1.4); 3.022(2.7); 3.014(3.4); 3.009(3.7);
3.003(3.1); 2.997(2.7); 2.993(2.5); 2.982(1.4); 2.676(1.5); 2.672(1.9); 2.667(1.3); 2.542(1.5); 2.511(134.9); 2.507(252.5); 2.503(317.1); 2.498(225.9); 2.494(107.9); 2.444(0.5);
2.440(0.5); 2.334(1.4); 2.329(1.9); 2.325(1.3); 2.097(2.3); 2.089(2.4); 2.078(4.1); 2.072(3.3); 2.067(2.7); 2.058(2.6); 2.050(2.5); 1.283(0.7); 1.269(7.8); 1.257(5.6); 1.249(14.9);
1.234(7.3); 1.217(0.7); 0.000(11.1); −0.009(0.4)
Example 222: ¹H-NMR(400.0 MHz. DMSO): δ = 8.871(4.1); 8.861(4.0); 8.315(0.6); 7.724(2.7); 7.718(3.8); 7.704(5.0); 7.659(1.2); 7.650(2.0); 7.644(3.0); 7.636(5.6); 7.631(3.3);
7.616(16.0); 7.606(6.3); 7.588(0.4); 7.455(3.7); 7.316(8.2); 7.302(4.6); 7.283(11.6); 7.265(8.7); 7.194(4.2); 7.176(11.1); 7.169(12.6); 7.166(11.8); 7.157(3.9); 7.149(9.5); 3.902(2.0);
3.325(315.2); 3.263(0.4); 3.044(1.1); 3.033(2.7); 3.024(3.7); 3.013(3.7); 3.005(2.8); 2.993(1.1); 2.675(1.2); 2.670(1.7); 2.666(1.2); 2.541(1.5); 2.506(216.6); 2.501(272.0); 2.497(196.9);
2.493(97.7); 2.333(1.3); 2.328(1.6); 2.324(1.2); 2.122(1.8); 2.114(2.0); 2.107(2.5); 2.098(4.0); 2.090(2.4); 2.083(2.2); 2.075(2.0); 1.335(2.1); 1.322(3.8); 1.310(3.8); 1.299(3.5);
1.286(2.4); 1.247(2.7); 1.232(4.4); 1.228(3.6); 1.217(2.8); 1.213(4.0); 1.198(1.9); 0.000(6.5)
Example 223: ¹H-NMR(400.0 MHz. DMSO): δ = 9.126(5.3); 9.116(5.3); 8.759(8.3); 8.747(8.7); 8.314(1.0); 7.909(11.4); 7.896(10.9); 7.404(0.4); 7.347(0.5); 7.326(0.3); 7.307(5.7);
7.289(15.0); 7.270(11.5); 7.206(5.4); 7.188(8.0); 7.169(16.0); 7.150(11.1); 7.102(0.3); 3.902(2.0); 3.466(0.3); 3.434(0.5); 3.426(0.5); 3.403(0.7); 3.386(1.3); 3.329(699.5); 3.261(0.5);
3.168(0.4); 3.023(1.3); 3.012(3.2); 3.003(4.4); 2.993(4.4); 2.984(3.2); 2.973(1.4); 2.676(1.9); 2.671(2.4); 2.666(1.9); 2.541(2.4); 2.511(171.2); 2.507(326.2); 2.502(415.0); 2.498(301.6);
2.493(149.6); 2.333(1.8); 2.329(2.4); 2.324(1.8); 2.038(2.4); 1.296(2.4); 1.281(5.3); 1.265(4.7); 1.262(5.4); 1.246(3.4); 1.237(3.2); 1.225(3.9); 1.212(4.2); 1.201(3.7); 1.187(1.8);
0.000(4.7)
Example 224: ¹H-NMR(400.0 MHz. DMSO): δ = 8.924(5.1); 8.914(5.0); 8.667(7.0); 8.663(7.3); 8.655(7.4); 8.651(6.8); 8.311(2.0); 8.172(0.5); 8.151(0.5); 8.099(0.6); 8.078(0.6);
7.923(6.6); 7.919(6.6); 7.904(7.5); 7.900(6.8); 7.751(0.4); 7.732(0.5); 7.711(0.4); 7.568(0.4); 7.549(0.5); 7.530(0.4); 7.523(0.3); 7.512(6.4); 7.500(6.5); 7.492(6.0); 7.480(5.8);
7.408(0.4); 7.356(0.4); 7.336(0.6); 7.305(5.7); 7.286(14.8); 7.267(10.9); 7.197(5.1); 7.179(7.7); 7.163(14.4); 7.160(16.0); 7.142(11.2); 6.637(1.3); 4.123(1.1); 4.115(1.9); 4.104(2.0);
4.094(1.7); 4.086(5.6); 4.076(5.8); 4.058(5.8); 4.047(6.0); 4.039(1.4); 4.030(2.0); 4.019(2.0); 4.011(1.0); 3.982(0.3); 3.900(2.1); 3.398(0.5); 3.381(0.7); 3.327(496.2); 3.270(0.7);
3.250(0.4); 3.041(1.3); 3.030(3.2); 3.021(4.4); 3.010(4.4); 3.002(3.3); 2.990(1.4); 2.676(1.5); 2.671(2.1); 2.667(1.5); 2.542(1.4); 2.511(148.5); 2.507(281.7); 2.502(356.2); 2.498(255.2);
2.494(123.7); 2.455(0.9); 2.334(1.7); 2.329(2.2); 2.325(1.5); 2.084(2.2); 2.076(2.4); 2.068(2.9); 2.060(4.7); 2.052(2.8); 2.045(2.7); 2.036(2.4); 1.306(2.0); 1.293(4.7); 1.281(4.4);
1.269(4.9); 1.263(4.4); 1.257(3.9); 1.248(5.5); 1.244(4.9); 1.229(4.8); 1.214(1.9); 0.000(11.3); −0.009(0.4)
Example 225: ¹H-NMR(400.0 MHz. DMSO): δ = 8.993(5.2); 8.983(5.1); 8.783(6.7); 8.779(7.2); 8.771(7.2); 8.767(6.8); 8.315(0.8); 8.038(5.7); 8.018(6.3); 7.677(4.7); 7.665(4.9);
7.658(4.5); 7.646(4.1); 7.340(0.4); 7.324(5.3); 7.305(5.6); 7.287(14.6); 7.268(11.1); 7.199(5.6); 7.196(5.1); 7.189(12.7); 7.180(9.8); 7.175(16.0); 7.171(15.0); 7.162(4.8); 7.154(12.0);
7.095(0.3); 7.078(0.4); 7.054(5.7); 3.902(2.3); 3.325(244.1); 3.268(0.3); 3.042(1.4); 3.031(3.3); 3.022(4.7); 3.012(4.7); 3.003(3.4); 2.992(1.5); 2.675(1.6); 2.671(2.1); 2.666(1.5);
2.569(0.5); 2.541(2.1); 2.510(143.5); 2.506(272.7); 2.502(346.4); 2.497(249.7); 2.493(121.9); 2.448(0.6); 2.333(1.5); 2.328(2.0); 2.324(1.5); 2.139(2.4); 2.131(2.5); 2.124(3.0);
2.116(5.0); 2.107(2.9); 2.100(2.8); 2.092(2.5); 1.324(2.4); 1.311(4.7); 1.299(4.8); 1.287(4.5); 1.275(3.4); 1.266(3.7); 1.250(5.5); 1.246(4.9); 1.236(3.5); 1.231(5.1); 1.217(2.2);
0.008(0.5); 0.000(12.0)
Example 226: ¹H-NMR(400.0 MHz. DMSO): δ = 9.033(6.4); 9.022(6.2); 8.950(0.5); 8.325(0.5); 8.315(0.8); 8.305(0.6); 8.263(8.4); 8.243(10.2); 8.069(13.5); 8.050(11.2); 7.867(0.6);
7.847(0.5); 7.372(0.3); 7.344(0.4); 7.309(6.0); 7.290(15.7); 7.272(11.9); 7.205(5.6); 7.186(9.7); 7.179(16.0); 7.176(15.9); 7.158(12.6); 7.060(0.7); 7.043(1.1); 6.630(0.6); 6.616(0.7);
3.902(2.7); 3.433(0.3); 3.403(0.5); 3.389(0.7); 3.325(487.7); 3.064(1.6); 3.053(3.0); 3.046(3.4); 3.040(4.8); 3.029(3.8); 3.024(2.8); 3.014(1.6); 2.675(2.0); 2.671(2.5); 2.609(0.3);
2.566(0.8); 2.541(2.3); 2.506(354.7); 2.502(440.6); 2.497(323.4); 2.406(0.4); 2.333(2.1); 2.329(2.7); 2.324(2.0); 2.134(2.5); 2.126(2.7); 2.115(5.0); 2.107(4.2); 2.095(3.0); 2.087(2.7);
1.282(10.9); 1.263(12.5); 1.247(10.1); 1.220(0.5); 1.210(0.5); 1.194(0.5); 1.176(0.4); 0.000(9.3)
Example 227: ¹H-NMR(400.0 MHz. DMSO): δ = 8.645(3.9); 8.627(4.1); 8.546(0.3); 8.392(0.4); 8.311(0.5); 7.638(0.5); 7.613(0.6); 7.594(0.5); 7.548(5.4); 7.529(6.7); 7.448(0.6);
7.430(0.5); 7.416(0.4); 7.386(0.6); 7.369(0.6); 7.355(0.7); 7.335(1.8); 7.318(4.6); 7.299(9.4); 7.282(11.8); 7.268(6.1); 7.264(6.5); 7.249(2.6); 7.224(0.6); 7.205(0.5); 7.193(0.5);
7.173(0.4); 7.107(0.4); 7.074(0.7); 7.048(1.7); 7.037(6.3); 7.017(11.0); 6.996(5.8); 6.801(4.3); 6.797(5.3); 6.779(5.1); 4.810(2.3); 4.791(3.4); 4.774(2.6); 4.205(0.3); 4.193(1.6);
4.172(4.1); 4.152(4.6); 4.132(2.3); 4.097(0.8); 4.075(0.7); 4.024(0.4); 4.011(0.4); 4.000(0.4); 3.984(0.4); 3.959(0.4); 3.902(4.0); 3.876(0.4); 3.854(0.4); 3.821(0.4); 3.773(0.5);
3.744(0.6); 3.735(0.5); 3.703(0.5); 3.689(0.6); 3.679(0.5); 3.670(0.6); 3.632(0.8); 3.603(1.0); 3.583(1.0); 3.572(1.0); 3.547(0.9); 3.498(2.1); 3.475(2.1); 3.433(4.2); 3.355(2013.6);
3.243(1.2); 3.231(1.2); 3.200(0.6); 3.169(16.0); 2.673(2.4); 2.620(0.8); 2.597(2.3); 2.580(3.0); 2.564(3.3); 2.544(2.5); 2.504(405.7); 2.420(2.8); 2.403(3.0); 2.381(1.9); 2.358(1.3);
2.327(4.9); 2.313(4.2); 2.297(5.3); 2.281(3.9); 2.269(2.3); 2.255(1.7); 2.240(0.9); 2.198(0.4); 1.827(0.3); 1.808(0.3); 1.794(0.4); 1.663(0.3); 1.617(0.5); 1.604(0.4); 1.234(0.6);
1.158(0.4); 1.149(0.4); 1.141(0.3); 0.798(0.6); 0.781(1.0); 0.762(0.7); 0.000(8.1)
Example 228: ¹H-NMR(400.0 MHz. DMSO): δ = 8.962(0.7); 8.936(0.4); 8.864(16.0); 8.847(5.0); 8.571(0.7); 8.447(10.9); 8.306(5.2); 7.414(0.4); 7.315(0.4); 7.300(1.5); 7.283(3.1);
7.280(3.1); 7.263(5.9); 7.246(3.4); 7.243(3.5); 7.226(1.7); 7.072(0.6); 7.051(0.8); 7.032(0.6); 7.009(1.2); 6.997(8.1); 6.976(14.2); 6.956(6.6); 6.942(1.0); 4.906(0.9); 4.887(2.9);
4.869(3.9); 4.852(2.9); 4.834(0.9); 4.224(1.9); 4.205(5.2); 4.184(5.3); 4.164(2.0); 3.897(3.1); 3.768(0.3); 3.589(0.4); 3.523(0.4); 3.511(0.5); 3.473(0.6); 3.444(0.5); 3.415(1.0);
3.398(1.5); 3.390(1.6); 3.334(525.4); 3.327(564.6); 2.700(0.3); 2.677(2.5); 2.673(3.1); 2.668(2.4); 2.639(0.5); 2.610(1.1); 2.584(2.4); 2.568(4.3); 2.543(5.0); 2.526(11.9); 2.513(209.0);
2.508(405.9); 2.504(518.6); 2.499(367.6); 2.495(173.5); 2.451(2.4); 2.432(0.9); 2.392(1.2); 2.370(2.2); 2.355(3.0); 2.335(4.1); 2.330(5.0); 2.326(3.6); 2.311(0.9); 2.285(1.7); 2.272(2.7);
2.259(3.0); 2.245(3.5); 2.232(2.3); 2.219(1.8); 2.205(0.8); 1.337(1.2); 1.299(2.4); 1.261(3.6); 1.251(2.1); 1.235(3.8); 1.189(0.3); 0.878(0.5); 0.865(0.6); 0.854(0.9); 0.836(0.6);
0.799(0.4); 0.780(0.7); 0.761(0.4); 0.008(0.7); 0.000(20.1); −0.009(0.5)
Example 229: ¹H-NMR(400.0 MHz. DMSO): δ = 8.717(6.7); 8.705(6.7); 8.316(1.0); 7.890(10.1); 7.888(10.7); 7.870(10.9); 7.868(10.9); 7.466(4.5); 7.464(4.7); 7.448(11.4);
7.444(16.0); 7.440(9.5); 7.429(7.9); 7.424(12.7); 7.421(11.1); 7.354(10.3); 7.350(11.5); 7.335(8.0); 7.331(7.7); 7.319(0.4); 7.316(0.4); 7.308(3.0); 7.305(3.2); 7.290(8.6); 7.287(8.4);
7.271(7.4); 7.268(6.6); 7.245(6.2); 7.240(7.8); 7.226(7.7); 7.221(8.8); 7.207(3.5); 7.202(3.4); 7.193(5.9); 7.189(5.9); 7.174(8.9); 7.170(8.6); 7.160(9.9); 7.155(13.9); 7.151(5.9);
7.141(7.3); 7.136(6.7); 4.038(0.4); 4.020(0.4); 3.322(68.3); 3.167(1.8); 3.156(4.2); 3.147(5.3); 3.135(5.4); 3.126(4.3); 3.114(1.9); 2.679(0.6); 2.675(1.1); 2.670(1.5); 2.666(1.1);
2.662(0.6); 2.524(4.6); 2.510(85.3); 2.506(173.3); 2.501(229.1); 2.497(164.9); 2.492(78.7); 2.399(2.9); 2.390(3.1); 2.383(3.7); 2.375(6.2); 2.367(3.6); 2.360(3.4); 2.351(3.2); 2.337(0.5);
2.333(1.1); 2.328(1.5); 2.323(1.1); 1.989(1.5); 1.390(3.5); 1.376(6.1); 1.365(6.4); 1.353(5.8); 1.340(3.9); 1.260(4.2); 1.245(6.4); 1.241(5.6); 1.230(4.5); 1.225(6.2); 1.211(3.3);
1.193(0.5); 1.175(0.9); 1.157(0.4); 0.146(0.4); 0.008(3.2); 0.000(98.3); −0.009(3.2); −0.150(0.4)
Example 230: ¹H-NMR(400.0 MHz. DMSO): δ = 8.847(9.5); 8.835(9.6); 8.317(0.6); 7.798(11.2); 7.779(14.7); 7.750(4.8); 7.733(12.4); 7.714(9.2); 7.672(8.7); 7.653(11.1); 7.634(4.2);
7.588(0.6); 7.583(0.4); 7.557(13.5); 7.539(11.1); 7.446(12.6); 7.442(12.9); 7.426(15.9); 7.423(16.0); 7.307(4.5); 7.304(4.9); 7.289(12.9); 7.285(12.7); 7.270(10.9); 7.266(9.8);
7.245(9.3); 7.241(11.5); 7.226(11.3); 7.222(12.9); 7.207(5.0); 7.203(4.7); 7.150(13.6); 7.146(13.1); 7.131(10.6); 7.127(10.0); 3.323(77.0); 3.166(2.5); 3.154(6.1); 3.145(7.8); 3.134(8.0);
3.125(6.4); 3.113(2.7); 2.675(1.1); 2.670(1.6); 2.666(1.2); 2.661(0.6); 2.524(4.5); 2.519(6.8); 2.510(86.4); 2.506(176.5); 2.501(234.7); 2.497(170.5); 2.492(82.8); 2.332(5.3); 2.323(5.5);
2.316(5.6); 2.307(9.0); 2.299(5.3); 2.292(5.0); 2.283(4.7); 1.989(0.5); 1.975(0.4); 1.349(4.8); 1.335(9.1); 1.324(9.1); 1.312(8.5); 1.299(6.1); 1.283(0.4); 1.271(6.6); 1.255(9.6);
1.251(8.7); 1.240(6.2); 1.235(9.5); 1.221(4.5); 0.146(0.5); 0.008(3.5); 0.000(106.7); −0.009(3.8); −0.150(0.5)
Example 231: ¹H-NMR(400.0 MHz. DMSO): δ = 8.505(1.8); 8.493(1.8); 8.252(4.1); 7.455(1.5); 7.438(2.3); 7.434(2.3); 7.418(2.9); 7.415(2.7); 7.320(3.4); 7.302(0.8); 7.299(0.9);
7.284(2.3); 7.280(2.2); 7.265(2.0); 7.262(1.7); 7.238(1.7); 7.234(2.0); 7.219(2.1); 7.215(2.2); 7.200(1.0); 7.196(0.9); 7.184(1.7); 7.155(2.4); 7.151(2.2); 7.136(1.8); 7.132(1.6);
4.038(0.8); 4.020(0.8); 3.922(16.0); 3.325(11.5); 3.170(0.4); 3.158(1.1); 3.149(1.4); 3.138(1.4); 3.129(1.1); 3.117(0.4); 2.511(16.7); 2.507(31.5); 2.502(39.8); 2.498(28.4); 2.494(13.7);
2.312(0.8); 2.302(0.8); 2.296(1.0); 2.287(1.6); 2.279(1.0); 2.272(0.9); 2.263(0.8); 1.989(3.5); 1.368(0.8); 1.355(1.5); 1.343(1.6); 1.331(1.5); 1.318(0.9); 1.227(1.0); 1.212(1.6);
1.208(1.3); 1.193(2.4); 1.175(2.1); 1.157(0.9); 0.008(0.8); 0.000(16.6); −0.009(0.6)
Example 232: ¹H-NMR(400.0 MHz. DMSO): δ = 8.715(6.7); 8.701(6.8); 8.316(0.9); 7.891(10.7); 7.873(10.9); 7.871(11.0); 7.697(8.3); 7.677(9.5); 7.630(3.7); 7.611(8.3); 7.592(5.1);
7.473(4.4); 7.471(4.6); 7.455(10.3); 7.452(10.7); 7.436(6.8); 7.434(6.9); 7.423(5.3); 7.404(8.5); 7.385(3.8); 7.336(9.9); 7.332(11.2); 7.317(16.0); 7.314(10.6); 7.299(8.1); 7.196(5.4);
7.192(5.4); 7.177(8.5); 7.173(8.4); 7.158(4.6); 7.154(4.4); 4.038(0.7); 4.020(0.7); 3.322(63.8); 3.306(1.8); 3.294(3.7); 3.285(4.6); 3.273(4.7); 3.264(3.7); 3.252(1.6); 2.675(1.2);
2.671(1.7); 2.666(1.2); 2.524(4.7); 2.506(188.1); 2.502(247.3); 2.497(181.8); 2.333(3.9); 2.328(4.9); 2.324(4.9); 1.989(3.1); 1.420(2.8); 1.406(6.1); 1.394(5.5); 1.383(5.8); 1.370(3.8);
1.356(3.7); 1.340(6.1); 1.320(5.9); 1.306(2.4); 1.193(0.9); 1.175(1.7); 1.157(0.9); 0.146(0.4); 0.008(3.1); 0.000(92.1); −0.008(3.3); −0.150(0.4)
Example 233: ¹H-NMR(400.0 MHz. DMSO): δ = 8.861(0.3); 8.832(6.3); 8.819(6.2); 8.315(1.2); 7.798(7.5); 7.778(9.9); 7.756(3.3); 7.738(8.4); 7.720(6.1); 7.698(7.9); 7.676(12.4);
7.654(7.5); 7.635(3.0); 7.626(3.7); 7.607(8.0); 7.587(4.9); 7.536(8.9); 7.517(7.4); 7.421(4.8); 7.402(8.0); 7.383(3.6); 7.286(8.6); 7.267(7.6); 3.902(4.1); 3.421(0.4); 3.399(0.7);
3.377(0.9); 3.367(1.4); 3.325(738.7); 3.294(2.8); 3.280(4.0); 3.268(5.1); 3.261(4.4); 3.254(4.1); 3.239(1.8); 2.675(2.3); 2.671(3.3); 2.666(2.4); 2.568(0.7); 2.541(2.7); 2.510(212.9);
2.506(407.2); 2.502(518.0); 2.497(371.9); 2.493(180.0); 2.333(2.3); 2.329(3.0); 2.324(2.2); 2.255(4.0); 1.381(11.3); 1.367(7.5); 1.361(16.0); 1.345(9.8); 1.236(0.7); 0.000(14.1)
Example 234: ¹H-NMR(400.0 MHz. DMSO): δ = 8.474(1.7); 8.462(1.7); 8.238(4.1); 7.689(2.1); 7.670(2.3); 7.623(0.9); 7.604(2.1); 7.584(1.2); 7.440(1.6); 7.418(1.4); 7.399(2.2);
7.380(1.0); 7.326(2.2); 7.305(5.4); 7.169(1.8); 5.757(3.2); 3.923(16.0); 3.326(7.3); 3.311(0.4); 3.299(0.9); 3.290(1.2); 3.278(1.2); 3.269(1.0); 3.257(0.4); 2.526(0.6); 2.521(1.0);
2.512(12.2); 2.508(24.8); 2.503(32.5); 2.498(23.2); 2.494(11.0); 2.230(0.9); 2.227(0.9); 2.218(0.7); 1.408(0.8); 1.394(1.5); 1.382(1.3); 1.371(1.4); 1.357(0.9); 1.326(0.9); 1.310(1.5);
1.306(1.2); 1.295(0.9); 1.291(1.5); 1.276(0.6); 0.008(0.6); 0.000(18.9); −0.009(0.6)
Example 235: ¹H-NMR(400.0 MHz. DMSO): δ = 9.064(0.4); 9.043(0.4); 8.967(0.3); 8.947(0.4); 8.813(0.6); 8.801(0.7); 8.786(0.7); 8.773(0.6); 8.717(7.5); 8.708(7.4); 8.665(5.3);
8.644(5.3); 8.315(1.5); 7.919(0.9); 7.900(1.1); 7.877(0.6); 7.855(0.7); 7.825(0.6); 7.812(0.8); 7.804(0.6); 7.792(0.6); 7.780(0.7); 7.768(0.6); 7.760(0.7); 7.742(3.3); 7.725(11.3);
7.694(11.6); 7.686(7.7); 7.673(16.0); 7.667(8.0); 7.654(6.5); 7.474(4.0); 7.457(6.5); 7.438(3.3); 7.257(7.5); 7.255(7.6); 7.237(6.9); 4.844(2.3); 4.827(3.5); 4.809(2.8); 4.788(1.0);
4.205(1.3); 4.185(3.6); 4.167(3.6); 4.147(1.4); 3.902(5.8); 3.359(0.9); 3.323(409.5); 3.263(0.6); 3.169(2.3); 2.675(2.7); 2.671(3.5); 2.666(2.5); 2.564(1.0); 2.541(3.9); 2.510(245.7);
2.506(462.8); 2.502(584.7); 2.497(420.3); 2.493(205.3); 2.463(3.6); 2.453(4.4); 2.434(2.1); 2.378(0.6); 2.333(2.9); 2.328(3.7); 2.324(2.7); 2.271(1.2); 2.255(1.9); 2.248(2.7); 2.243(2.7);
2.234(3.3); 2.228(3.5); 2.222(2.7); 2.213(2.3); 2.206(2.9); 2.185(1.3); 2.142(0.6); 2.087(1.7); 2.078(1.2); 2.069(2.2); 2.063(2.4); 2.046(3.4); 2.034(3.2); 2.024(2.2); 2.002(1.3);
1.983(0.6); 1.949(0.4); 1.942(0.4); 1.924(0.4); 1.918(0.6); 1.892(0.4); 1.872(0.4); 1.834(0.4); 1.819(0.4); 1.753(0.4); 1.727(0.4); 1.701(0.3); 1.623(0.3); 1.617(0.4); 1.589(0.5);
1.566(0.5); 1.540(0.4); 1.521(0.4); 1.512(0.4); 1.494(0.4); 1.441(0.6); 1.391(0.4); 1.350(0.4); 1.340(0.4); 1.318(0.5); 1.296(0.6); 1.276(0.4); 1.260(0.4); 1.235(0.7); 0.000(18.3); −0.009
(0.7)
Example 236: ¹H-NMR(400.0 MHz. DMSO): δ = 8.882(5.0); 8.863(5.0); 8.791(0.3); 8.726(7.1); 8.716(7.4); 8.637(0.3); 8.315(0.9); 7.692(5.5); 7.680(5.6); 7.673(6.2); 7.661(6.0);
7.646(0.5); 7.634(0.3); 7.442(0.4); 7.358(1.4); 7.342(3.2); 7.337(3.1); 7.321(6.0); 7.304(3.7); 7.300(5.1); 7.294(7.9); 7.284(2.6); 7.275(6.7); 7.242(0.5); 7.221(0.5); 7.160(0.3);
7.146(0.5); 7.129(0.6); 7.112(0.3); 7.072(1.4); 7.061(9.2); 7.040(16.0); 7.019(7.5); 7.006(1.1); 4.850(0.8); 4.831(2.8); 4.815(3.9); 4.797(2.9); 4.777(0.9); 4.204(1.9); 4.183(4.9);
4.163(5.0); 4.143(1.9); 4.102(0.4); 4.085(0.4); 3.902(3.0); 3.323(427.9); 3.259(0.4); 3.168(1.9); 2.675(2.1); 2.671(2.8); 2.666(2.1); 2.628(0.9); 2.602(2.2); 2.585(3.6); 2.557(3.1);
2.541(4.3); 2.510(198.5); 2.506(370.7); 2.502(467.6); 2.497(338.6); 2.459(3.5); 2.431(3.0); 2.415(1.4); 2.379(1.6); 2.356(2.4); 2.338(3.6); 2.333(4.3); 2.328(4.6); 2.324(3.8); 2.319(3.1);
2.294(1.0); 2.252(1.9); 2.237(2.7); 2.224(3.3); 2.211(3.7); 2.197(2.7); 2.184(1.8); 2.170(0.9); 2.081(0.3); 0.000(10.2)
Example 237: ¹H-NMR(400.0 MHz. DMSO): δ = 9.218(6.8); 9.197(7.0); 8.963(16.0); 8.753(0.3); 8.590(15.1); 8.444(0.4); 8.310(5.0); 7.612(8.4); 7.593(10.0); 7.411(10.1); 7.408(9.2);
7.391(13.2); 7.389(12.5); 7.381(5.6); 7.362(11.3); 7.343(6.6); 7.273(7.1); 7.270(6.4); 7.254(9.5); 7.236(3.9); 7.232(3.3); 7.222(0.4); 4.799(1.2); 4.776(4.6); 4.754(6.6); 4.732(4.7);
4.710(1.4); 3.899(4.9); 3.801(2.6); 3.777(6.5); 3.754(6.8); 3.731(2.6); 3.508(0.4); 3.468(0.4); 3.439(0.6); 3.420(0.5); 3.388(1.4); 3.325(782.8); 3.256(0.5); 3.237(0.5); 3.223(0.5);
3.175(0.4); 2.749(0.4); 2.734(0.6); 2.713(0.4); 2.676(3.2); 2.672(4.1); 2.668(3.0); 2.621(0.5); 2.566(1.2); 2.542(3.7); 2.507(568.2); 2.503(713.8); 2.498(514.3); 2.416(0.6); 2.357(0.4);
2.334(3.5); 2.329(4.9); 2.325(4.7); 2.301(5.5); 2.276(8.2); 2.256(7.3); 2.235(5.9); 2.214(2.4); 2.114(1.7); 2.088(4.9); 2.064(6.7); 2.041(4.1); 2.015(1.3); 1.670(1.7); 1.644(5.1);
1.623(5.6); 1.618(5.5); 1.597(4.3); 1.571(1.4); 1.260(0.4); 1.238(0.4); 0.000(22.8)
Example 238: ¹H-NMR(400.0 MHz. DMSO): δ = 9.078(6.5); 9.056(6.6); 8.542(14.4); 8.539(14.8); 8.530(15.1); 8.527(14.5); 8.313(3.6); 8.026(14.3); 8.023(14.2); 8.005(16.0);
8.002(14.9); 7.614(8.6); 7.611(8.8); 7.595(10.4); 7.592(9.9); 7.547(15.1); 7.535(14.6); 7.526(13.8); 7.515(13.7); 7.501(0.4); 7.466(0.3); 7.406(10.9); 7.403(11.6); 7.387(13.9);
7.384(15.3); 7.373(5.7); 7.370(5.6); 7.355(12.4); 7.352(10.8); 7.336(7.4); 7.333(6.0); 7.267(7.5); 7.263(7.5); 7.248(10.3); 7.244(9.7); 7.229(4.3); 7.225(3.9); 4.802(1.5); 4.780(5.1);
4.758(7.3); 4.736(5.1); 4.714(1.5); 3.901(9.2); 3.814(2.9); 3.791(6.9); 3.768(7.0); 3.744(2.8); 3.733(0.4); 3.710(0.4); 3.525(0.4); 3.509(0.4); 3.476(0.6); 3.469(0.5); 3.452(0.6);
3.435(0.8); 3.417(1.1); 3.407(1.1); 3.395(1.5); 3.330(1253.4); 3.276(1.7); 3.259(0.7); 3.219(0.3); 3.163(0.3); 2.995(0.3); 2.746(0.4); 2.728(0.6); 2.709(0.5); 2.676(3.0); 2.672(4.0);
2.667(2.9); 2.572(0.7); 2.562(0.7); 2.542(3.3); 2.525(12.2); 2.512(269.8); 2.507(527.6); 2.503(676.0); 2.498(481.5); 2.494(229.0); 2.338(1.4); 2.334(3.0); 2.329(4.0); 2.325(2.9);
2.320(1.7); 2.313(2.1); 2.290(5.6); 2.266(6.0); 2.246(3.7); 2.226(5.3); 2.205(6.3); 2.185(2.6); 2.132(2.0); 2.107(5.4); 2.083(7.3); 2.059(4.5); 2.034(1.5); 1.633(2.0); 1.607(5.7);
1.587(6.2); 1.581(6.4); 1.561(5.2); 1.555(2.8); 1.534(1.5); 1.300(0.4); 1.259(0.4); 1.245(0.4); 1.239(0.5); 1.234(0.6); 0.000(13.1)
Example 239: ¹H-NMR(400.0 MHz. DMSO): δ = 8.876(2.4); 8.854(2.5); 8.315(0.6); 7.621(3.1); 7.618(3.2); 7.601(3.7); 7.598(3.6); 7.488(3.1); 7.469(6.2); 7.451(2.3); 7.443(2.9);
7.437(2.8); 7.429(3.9); 7.424(1.4); 7.417(2.0); 7.409(6.3); 7.406(4.7); 7.398(2.3); 7.388(16.0); 7.380(5.8); 7.374(5.3); 7.357(4.7); 7.338(2.7); 7.335(2.3); 7.268(2.6); 7.264(2.6);
7.248(3.6); 7.245(3.5); 7.229(1.6); 7.226(1.5); 4.740(0.5); 4.718(1.8); 4.697(2.5); 4.675(1.8); 4.652(0.6); 3.902(0.5); 3.768(1.0); 3.744(2.4); 3.721(2.5); 3.698(1.0); 3.372(0.3);
3.354(1.0); 3.325(310.5); 3.282(0.5); 3.263(0.6); 3.248(0.4); 2.723(0.3); 2.675(0.9); 2.671(1.3); 2.666(0.9); 2.541(1.1); 2.524(4.0); 2.511(85.9); 2.506(168.6); 2.502(217.8);
2.497(157.9); 2.493(77.8); 2.333(1.0); 2.328(1.4); 2.324(1.0); 2.290(0.8); 2.267(2.0); 2.243(2.5); 2.229(2.1); 2.208(2.2); 2.187(0.9); 2.076(0.7); 2.050(1.9); 2.026(2.7); 2.003(1.6);
1.977(0.5); 1.662(0.7); 1.635(2.1); 1.615(2.2); 1.610(2.2); 1.589(1.7); 1.584(1.0); 1.562(0.6); 0.000(5.2)
Example 240: ¹H-NMR(400.0 MHz. DMSO): δ = 8.930(3.9); 8.908(3.8); 8.304(2.4); 7.659(7.0); 7.654(6.4); 7.602(5.4); 7.585(6.3); 7.484(3.6); 7.463(8.0); 7.432(16.0); 7.412(7.6);
7.401(5.9); 7.381(7.5); 7.368(3.2); 7.349(6.6); 7.330(3.8); 7.262(4.0); 7.243(5.6); 7.224(2.3); 4.722(0.8); 4.699(2.9); 4.678(4.1); 4.656(2.9); 4.634(0.9); 3.897(2.0); 3.767(1.8);
3.743(4.2); 3.720(4.2); 3.697(1.6); 3.442(0.5); 3.391(1.2); 3.329(491.8); 3.327(486.2); 3.247(0.6); 3.233(0.3); 2.677(1.9); 2.673(2.5); 2.668(1.8); 2.612(0.4); 2.595(0.4); 2.567(0.8);
2.563(0.8); 2.543(1.9); 2.512(178.8); 2.508(335.1); 2.504(423.3); 2.499(303.8); 2.495(147.6); 2.425(0.3); 2.335(1.9); 2.330(2.5); 2.326(1.9); 2.294(1.3); 2.271(3.4); 2.247(4.1);
2.233(3.7); 2.212(3.8); 2.191(1.6); 2.072(1.1); 2.046(3.1); 2.022(4.3); 1.998(2.6); 1.973(0.8); 1.672(1.0); 1.646(3.1); 1.625(3.5); 1.620(3.5); 1.599(2.6); 1.573(0.9); 1.236(0.4);
0.000(9.4)
Example 241: ¹H-NMR(400.0 MHz. DMSO): δ = 16.900(0.4); 8.843(0.4); 8.811(8.1); 8.789(8.0); 8.367(0.6); 8.352(0.5); 8.339(0.4); 8.305(7.1); 7.917(0.4); 7.898(0.5); 7.869(15.0);
7.849(15.9); 7.782(0.3); 7.648(10.3); 7.632(12.0); 7.610(0.5); 7.579(0.4); 7.529(0.4); 7.503(0.7); 7.485(1.0); 7.447(6.8); 7.428(15.4); 7.409(10.4); 7.395(11.5); 7.373(16.0); 7.352(13.8);
7.333(7.9); 7.304(14.9); 7.300(15.8); 7.285(13.1); 7.281(12.2); 7.259(8.1); 7.240(11.1); 7.221(4.7); 7.194(1.0); 7.174(7.5); 7.170(6.9); 7.155(11.9); 7.152(11.1); 7.136(6.1); 7.132(5.6);
7.000(0.4); 4.746(1.5); 4.724(5.6); 4.703(8.3); 4.681(5.8); 4.659(1.6); 4.332(0.4); 3.998(0.3); 3.896(5.5); 3.866(0.3); 3.848(0.4); 3.823(0.4); 3.813(0.4); 3.802(0.4); 3.798(0.5);
3.770(3.4); 3.747(8.2); 3.723(8.3); 3.700(3.1); 3.678(0.4); 3.666(0.4); 3.656(0.4); 3.644(0.4); 3.627(0.4); 3.581(0.7); 3.567(0.7); 3.565(0.6); 3.551(0.7); 3.533(0.8); 3.508(1.0);
3.477(0.9); 3.463(0.9); 3.424(1.4); 3.331(992.3); 3.325(1129.4); 3.247(2.2); 3.232(1.8); 3.218(1.0); 3.178(0.4); 3.165(0.4); 3.058(0.4); 2.746(1.2); 2.728(1.7); 2.709(1.4); 2.677(4.5);
2.672(6.3); 2.668(4.5); 2.624(0.7); 2.602(0.8); 2.589(0.9); 2.543(4.6); 2.512(428.7); 2.508(818.4); 2.503(1039.7); 2.499(743.5); 2.495(357.9); 2.441(1.1); 2.422(0.9); 2.410(0.6);
2.402(0.5); 2.396(0.5); 2.335(4.6); 2.330(6.2); 2.326(4.8); 2.321(3.8); 2.294(6.4); 2.271(7.0); 2.249(3.9); 2.233(6.3); 2.211(7.2); 2.190(3.1); 2.173(0.4); 2.125(0.4); 2.107(0.4);
2.098(2.1); 2.072(6.2); 2.048(8.3); 2.024(5.3); 1.999(1.8); 1.863(0.3); 1.701(0.4); 1.674(0.6); 1.664(0.9); 1.640(2.6); 1.614(6.4); 1.593(7.4); 1.588(7.2); 1.567(5.7); 1.542(1.7);
1.511(0.3); 1.433(0.5); 1.302(0.4); 1.261(0.5); 1.237(0.9); 0.856(0.6); 0.837(0.4); 0.813(0.4); 0.000(31.4); −0.008(1.0)
Example 242: ¹H-NMR(400.0 MHz. DMSO): δ = 11.984(0.3); 8.930(8.3); 8.908(8.4); 8.885(0.5); 8.301(7.1); 7.892(0.4); 7.848(0.4); 7.827(0.4); 7.766(11.3); 7.746(15.2); 7.728(5.6);
7.709(13.8); 7.690(10.2); 7.650(10.0); 7.631(12.8); 7.612(5.0); 7.596(12.4); 7.577(14.7); 7.554(0.4); 7.540(0.5); 7.493(16.0); 7.474(13.5); 7.448(0.4); 7.431(0.6); 7.428(0.6);
7.402(12.3); 7.383(16.0); 7.363(7.3); 7.345(15.1); 7.326(8.7); 7.305(0.4); 7.284(0.5); 7.263(9.9); 7.244(13.3); 7.225(5.3); 7.192(0.3); 4.767(0.3); 4.756(2.0); 4.734(6.9); 4.712(10.3);
4.691(7.1); 4.668(1.9); 4.488(0.3); 4.418(0.4); 3.898(3.1); 3.894(3.2); 3.858(0.4); 3.846(0.3); 3.826(0.4); 3.820(0.4); 3.781(0.4); 3.775(0.4); 3.752(4.0); 3.728(10.0); 3.704(10.2);
3.681(4.1); 3.656(0.4); 3.596(0.5); 3.589(0.4); 3.579(0.4); 3.558(0.5); 3.531(0.7); 3.527(0.6); 3.512(0.9); 3.481(0.8); 3.437(1.0); 3.386(2.7); 3.331(803.4); 3.327(685.2); 3.323(733.7);
3.248(0.5); 3.231(0.4); 3.212(0.4); 3.181(0.3); 3.174(0.6); 2.922(0.4); 2.895(0.4); 2.869(0.4); 2.858(0.3); 2.795(0.4); 2.750(0.4); 2.678(5.0); 2.673(6.5); 2.669(4.7); 2.664(2.5);
2.598(1.2); 2.572(2.1); 2.568(2.1); 2.543(5.3); 2.513(441.4); 2.509(830.2); 2.504(1046.9); 2.499(744.4); 2.495(356.1); 2.369(0.3); 2.361(0.4); 2.335(4.6); 2.331(6.1); 2.326(4.4);
2.306(2.9); 2.284(7.8); 2.261(8.6); 2.235(9.0); 2.209(8.6); 2.188(3.6); 2.063(2.4); 2.038(7.4); 2.014(9.9); 1.989(6.3); 1.965(2.0); 1.955(0.4); 1.638(2.5); 1.613(7.2); 1.592(8.2);
1.587(7.9); 1.566(6.5); 1.540(1.7); 1.434(0.3); 1.303(0.5); 1.299(0.4); 1.262(0.5); 1.252(0.5); 1.236(1.1); 1.229(0.6); 1.164(0.4); 0.881(0.3); 0.855(0.6); 0.837(0.4); 0.828(0.5);
0.814(0.5); 0.008(2.0); 0.000(39.9); −0.008(1.3); −0.150(0.4); −2.858(0.3); −3.174(0.3); −3.439(0.3)
Example 243: ¹H-NMR(400.0 MHz. DMSO): δ = 9.148(4.7); 9.127(4.7); 8.690(0.4); 8.311(2.4); 7.591(6.7); 7.588(6.7); 7.572(8.2); 7.538(1.7); 7.522(3.8); 7.517(3.5); 7.501(6.8);
7.484(3.8); 7.479(4.2); 7.463(1.8); 7.412(7.9); 7.410(8.0); 7.393(10.2); 7.390(10.4); 7.378(4.5); 7.360(9.2); 7.341(5.2); 7.309(0.4); 7.291(0.6); 7.273(6.1); 7.269(5.6); 7.254(7.9);
7.251(7.2); 7.235(3.5); 7.231(3.0); 7.222(0.8); 7.201(0.6); 7.165(9.9); 7.145(16.0); 7.125(8.4); 4.740(1.1); 4.718(3.6); 4.697(5.1); 4.676(3.6); 4.654(1.1); 3.900(3.6); 3.738(2.1);
3.715(5.1); 3.692(5.4); 3.668(2.1); 3.597(0.3); 3.567(0.3); 3.467(0.4); 3.449(0.6); 3.428(0.6); 3.411(0.7); 3.397(0.8); 3.376(1.7); 3.327(689.9); 3.285(1.8); 3.269(1.2); 3.254(0.7);
2.739(0.5); 2.720(0.8); 2.701(0.7); 2.676(2.2); 2.672(3.0); 2.667(2.2); 2.620(0.4); 2.594(0.4); 2.542(2.8); 2.511(213.4); 2.507(405.3); 2.503(512.5); 2.498(367.5); 2.494(178.1);
2.462(1.2); 2.440(0.7); 2.334(2.4); 2.329(3.1); 2.325(2.3); 2.298(1.6); 2.275(5.6); 2.252(9.8); 2.230(6.3); 2.208(2.0); 2.039(1.1); 2.015(3.7); 1.991(5.7); 1.967(3.4); 1.942(1.1);
1.667(1.4); 1.641(3.9); 1.620(4.9); 1.615(4.6); 1.594(3.1); 1.568(1.2); 1.543(0.5); 1.538(0.4); 1.527(0.3); 1.337(0.5); 1.259(0.4); 1.235(1.0); 1.226(0.7); 0.855(0.3); 0.008(0.6);
0.000(15.2); −0.009(0.5)
Example 244: ¹H-NMR(400.0 MHz. DMSO): δ = 8.860(3.9); 8.838(3.9); 8.311(4.7); 7.641(8.5); 7.626(8.6); 7.623(8.9); 7.621(8.8); 7.609(5.8); 7.607(5.6); 7.440(2.4); 7.437(2.5);
7.424(3.2); 7.419(8.0); 7.403(11.6); 7.401(11.5); 7.384(7.5); 7.381(8.4); 7.373(4.0); 7.370(4.4); 7.363(16.0); 7.355(8.2); 7.352(7.6); 7.347(10.1); 7.345(10.9); 7.336(5.3); 7.333(4.3);
7.327(3.6); 7.322(2.4); 7.289(0.3); 7.274(0.7); 7.265(4.1); 7.261(4.1); 7.246(5.6); 7.242(5.4); 7.227(2.4); 7.223(2.4); 7.216(0.5); 4.743(0.8); 4.721(2.8); 4.699(4.0); 4.677(2.9);
4.655(0.8); 3.900(5.1); 3.765(1.6); 3.742(3.9); 3.719(4.0); 3.695(1.6); 3.509(0.4); 3.483(0.4); 3.451(0.5); 3.407(0.8); 3.329(774.2); 3.326(794.3); 3.273(0.6); 3.256(0.8); 3.241(0.7);
2.742(0.5); 2.724(0.8); 2.705(0.7); 2.676(2.6); 2.672(3.5); 2.667(2.6); 2.663(1.3); 2.542(2.6); 2.525(10.1); 2.511(220.7); 2.507(434.3); 2.503(560.4); 2.498(399.3); 2.494(189.6);
2.334(2.4); 2.329(3.3); 2.325(2.4); 2.320(1.1); 2.301(1.1); 2.278(3.1); 2.252(4.7); 2.233(4.5); 2.208(3.4); 2.187(1.4); 2.080(0.9); 2.054(3.0); 2.031(4.1); 2.007(2.5); 1.982(0.8);
1.650(1.3); 1.624(3.2); 1.604(3.4); 1.598(3.3); 1.578(3.0); 1.551(0.8); 1.259(0.3); 1.235(0.7); 1.226(0.4); 0.146(0.5); 0.008(4.0); 0.000(111.9); −0.008(3.7); −0.150(0.5)
Example 245: ¹H-NMR(400.0 MHz. DMSO): δ = 9.095(7.7); 9.073(7.6); 8.842(0.4); 8.795(10.2); 8.793(10.8); 8.784(10.4); 8.781(10.2); 8.315(2.7); 7.992(9.3); 7.989(9.5);
7.972(11.3); 7.970(11.0); 7.849(0.3); 7.784(10.0); 7.772(10.2); 7.765(8.6); 7.753(7.9); 7.603(9.2); 7.600(9.5); 7.584(11.0); 7.581(10.6); 7.491(0.3); 7.470(0.4); 7.442(0.4); 7.417(11.5);
7.414(12.3); 7.397(14.7); 7.394(16.0); 7.379(6.4); 7.376(6.2); 7.360(13.4); 7.357(11.7); 7.341(7.7); 7.338(6.5); 7.311(0.4); 7.276(8.1); 7.272(8.2); 7.257(10.9); 7.253(10.4); 7.237(4.6);
7.233(4.3); 4.750(1.5); 4.728(5.3); 4.706(7.7); 4.685(5.5); 4.663(1.6); 3.902(11.6); 3.742(3.1); 3.719(7.5); 3.696(7.7); 3.672(3.1); 3.584(0.4); 3.527(0.4); 3.519(0.5); 3.507(0.6);
3.494(0.5); 3.463(0.5); 3.447(0.6); 3.406(0.9); 3.325(1391.5); 3.272(0.6); 3.260(0.4); 2.694(0.3); 2.679(2.4); 2.675(4.6); 2.671(6.3); 2.666(4.5); 2.662(2.2); 2.617(0.4); 2.541(4.4);
2.524(21.0); 2.511(410.9); 2.506(788.8); 2.502(1009.1); 2.497(716.2); 2.493(338.6); 2.402(0.3); 2.337(2.2); 2.333(4.6); 2.328(6.2); 2.324(4.6); 2.319(2.6); 2.312(2.3); 2.290(6.2);
2.265(7.6); 2.251(6.3); 2.245(5.0); 2.231(6.5); 2.209(2.7); 2.047(1.9); 2.021(5.5); 1.998(8.2); 1.974(4.9); 1.949(1.6); 1.663(2.0); 1.642(3.2); 1.637(6.0); 1.617(6.6); 1.611(6.4);
1.591(5.2); 1.586(2.7); 1.564(1.5); 1.336(0.5); 1.298(0.5); 1.259(0.6); 1.249(0.4); 1.235(1.1); 1.225(0.7); 0.853(0.4); 0.836(0.4); 0.146(0.7); 0.008(7.5); 0.000(163.9); −0.009(5.5); −0.150
(0.7)
Example 246: ¹H-NMR(400.0 MHz. DMSO): δ = 8.857(15.7); 8.854(15.1); 8.595(7.2); 8.572(7.3); 8.451(16.0); 8.447(15.2); 8.315(2.7); 7.469(8.0); 7.465(8.2); 7.450(10.5);
7.446(10.2); 7.381(10.1); 7.378(10.7); 7.362(13.1); 7.359(13.8); 7.329(5.2); 7.326(5.2); 7.310(11.4); 7.307(10.2); 7.291(6.7); 7.288(5.6); 7.244(7.6); 7.240(7.7); 7.225(9.9); 7.221(9.4);
7.206(4.0); 7.203(3.7); 4.978(1.6); 4.964(3.6); 4.957(3.8); 4.942(3.5); 4.935(3.6); 4.912(1.3); 4.209(2.2); 4.189(5.9); 4.168(5.6); 4.148(2.2); 3.902(7.9); 3.323(907.0); 2.675(4.4);
2.671(5.9); 2.666(4.3); 2.541(4.5); 2.524(18.8); 2.511(381.1); 2.506(747.9); 2.502(963.7); 2.497(687.6); 2.493(329.7); 2.479(13.9); 2.469(6.4); 2.459(5.4); 2.440(2.1); 2.337(2.1);
2.333(4.4); 2.329(5.8); 2.324(4.1); 2.259(0.7); 2.235(2.6); 2.230(1.8); 2.223(2.3); 2.215(4.1); 2.208(4.3); 2.203(4.0); 2.195(2.9); 2.189(3.0); 2.174(1.2); 2.166(1.6); 2.040(1.6);
2.034(2.5); 2.023(1.9); 2.011(3.7); 2.004(4.8); 1.991(4.2); 1.966(1.6); 1.956(0.6); 1.336(0.3); 1.298(1.1); 1.259(1.5); 1.236(0.9); 0.146(1.1); 0.008(9.5); 0.000(256.4); −0.008(8.5); −0.149
(1.1)
Example 247: ¹H-NMR(400.0 MHz. DMSO): δ = 8.413(14.5); 8.409(13.7); 8.401(16.0); 8.398(14.7); 8.388(6.7); 8.366(6.4); 8.315(1.7); 7.895(14.2); 7.892(13.0); 7.875(15.8);
7.871(13.7); 7.543(0.3); 7.504(9.3); 7.501(9.2); 7.485(11.7); 7.482(10.8); 7.449(13.7); 7.438(13.2); 7.429(12.5); 7.417(12.2); 7.389(0.3); 7.364(11.1); 7.361(10.7); 7.344(14.5);
7.341(14.3); 7.329(6.0); 7.326(5.3); 7.310(12.8); 7.307(10.3); 7.291(7.5); 7.288(5.7); 7.234(8.1); 7.230(7.7); 7.215(10.8); 7.211(9.7); 7.196(4.4); 7.192(3.8); 4.960(1.5); 4.943(3.1);
4.938(4.1); 4.926(4.0); 4.921(4.1); 4.917(4.1); 4.908(4.0); 4.897(1.8); 4.888(1.5); 4.203(2.3); 4.182(6.1); 4.162(6.7); 4.143(2.5); 3.902(7.3); 3.322(271.0); 2.675(2.8); 2.671(3.7);
2.666(2.7); 2.541(2.9); 2.506(479.0); 2.502(592.6); 2.497(426.6); 2.470(13.6); 2.462(7.3); 2.453(6.0); 2.443(4.6); 2.426(1.9); 2.333(2.7); 2.328(3.5); 2.324(2.6); 2.236(2.4); 2.229(1.8);
2.221(2.1); 2.213(3.3); 2.207(3.8); 2.200(4.4); 2.194(4.3); 2.188(3.5); 2.181(2.6); 2.173(2.5); 2.161(1.2); 2.147(0.7); 2.103(0.4); 2.094(0.9); 2.082(2.8); 2.059(4.8); 2.049(5.4);
2.035(2.8); 2.023(1.5); 2.015(2.0); 2.010(1.5); 1.298(0.4); 1.259(0.6); 1.236(0.8); 0.146(0.5); 0.008(5.1); 0.000(122.7); −0.008(5.5); −0.150(0.5)
Example 248: ¹H-NMR(400.0 MHz. DMSO): δ = 8.315(4.6); 8.294(3.3); 7.506(4.3); 7.503(4.5); 7.487(5.8); 7.483(5.8); 7.411(5.5); 7.408(6.0); 7.392(7.1); 7.388(8.2); 7.376(5.6);
7.360(15.2); 7.357(16.0); 7.351(6.1); 7.338(8.7); 7.334(6.2); 7.318(2.3); 7.314(2.4); 7.281(4.1); 7.277(4.1); 7.262(5.5); 7.258(5.5); 7.252(4.2); 7.248(4.1); 7.243(2.8); 7.239(2.7);
7.233(5.4); 7.229(5.3); 7.216(3.0); 7.212(2.9); 6.787(5.9); 6.784(5.8); 6.768(5.7); 6.765(5.4); 4.944(0.7); 4.926(1.7); 4.922(2.0); 4.905(2.4); 4.891(1.8); 4.886(1.6); 4.870(0.8);
4.195(1.1); 4.174(2.9); 4.154(3.4); 4.135(1.3); 3.902(4.4); 3.321(188.9); 2.679(1.0); 2.675(2.0); 2.670(2.7); 2.666(2.0); 2.541(1.8); 2.524(7.6); 2.510(174.5); 2.506(347.8); 2.501(450.5);
2.497(322.0); 2.492(154.0); 2.466(4.2); 2.449(5.8); 2.433(4.3); 2.414(2.4); 2.396(0.9); 2.337(0.9); 2.333(1.9); 2.328(2.7); 2.323(1.9); 2.319(0.9); 2.221(1.0); 2.217(0.9); 2.199(2.2);
2.191(1.8); 2.184(2.1); 2.180(2.2); 2.173(1.4); 2.162(1.6); 2.158(1.3); 2.132(0.6); 2.094(0.5); 2.089(0.6); 2.077(1.1); 2.066(1.1); 2.055(2.4); 2.044(2.8); 2.030(1.4); 2.025(1.4);
2.017(0.7); 2.014(0.7); 2.005(0.7); 1.236(0.6); 0.852(0.4); 0.146(0.5); 0.008(4.5); 0.000(135.4); −0.009(4.4); −0.150(0.5)
Example 249: ¹H-NMR(400.0 MHz. DMSO): δ = 8.366(4.0); 8.344(4.0); 8.305(4.6); 8.301(7.1); 7.540(5.4); 7.535(9.7); 7.492(5.4); 7.489(5.5); 7.473(7.4); 7.470(7.1); 7.412(0.4);
7.395(6.6); 7.393(5.7); 7.376(9.0); 7.373(8.4); 7.368(4.7); 7.364(5.0); 7.359(6.7); 7.353(6.1); 7.350(8.4); 7.342(4.7); 7.338(7.0); 7.333(6.5); 7.271(4.7); 7.267(4.4); 7.252(6.4);
7.249(5.7); 7.233(2.5); 7.229(2.2); 6.830(16.0); 6.809(15.0); 4.935(0.9); 4.918(2.1); 4.913(2.6); 4.900(2.6); 4.896(2.8); 4.882(2.4); 4.872(1.1); 4.862(1.1); 4.194(1.5); 4.174(3.8);
4.153(4.2); 4.134(1.6); 3.894(4.0); 3.513(0.4); 3.477(0.4); 3.327(691.0); 3.323(655.9); 2.682(1.5); 2.678(3.0); 2.673(4.0); 2.669(3.0); 2.664(1.4); 2.628(0.4); 2.577(0.7); 2.543(2.8);
2.526(11.7); 2.513(262.8); 2.509(518.7); 2.504(668.5); 2.500(476.1); 2.495(225.9); 2.465(5.6); 2.456(5.8); 2.450(7.3); 2.439(3.4); 2.434(4.3); 2.419(2.7); 2.402(0.9); 2.340(1.4);
2.335(2.9); 2.331(3.9); 2.326(2.8); 2.227(1.3); 2.223(1.2); 2.204(2.5); 2.196(2.2); 2.191(2.6); 2.185(2.5); 2.175(1.6); 2.170(1.7); 2.163(1.6); 2.151(0.8); 2.137(0.5); 2.082(0.3);
2.075(0.5); 2.063(1.7); 2.041(2.8); 2.028(3.3); 2.012(1.7); 1.997(1.0); 1.992(0.8); 1.262(0.4); 1.236(0.6); 1.227(0.3); 0.146(0.7); 0.008(6.3); 0.000(179.4); −0.008(5.7); −0.150(0.7)
Example 250: ¹H-NMR(400.0 MHz. DMSO): δ = 8.830(0.4); 8.808(0.4); 8.316(2.7); 8.285(6.2); 8.263(6.2); 7.852(1.1); 7.835(1.4); 7.831(1.0); 7.785(11.1); 7.783(11.4); 7.766(12.0);
7.764(11.6); 7.598(0.6); 7.579(0.7); 7.537(0.4); 7.518(1.1); 7.508(7.3); 7.504(7.7); 7.489(9.9); 7.485(9.6); 7.472(1.2); 7.452(1.6); 7.435(0.8); 7.432(0.7); 7.413(9.0); 7.410(10.0);
7.394(11.9); 7.391(16.0); 7.386(5.9); 7.370(11.6); 7.367(9.3); 7.351(6.0); 7.348(4.8); 7.339(0.8); 7.320(0.5); 7.290(6.2); 7.288(7.5); 7.285(8.1); 7.281(7.7); 7.272(13.2); 7.269(14.3);
7.266(11.2); 7.262(9.3); 7.253(7.7); 7.250(8.0); 7.247(5.0); 7.243(3.7); 7.231(0.6); 7.086(6.5); 7.082(6.8); 7.067(9.9); 7.063(9.9); 7.048(5.4); 7.044(5.2); 6.587(10.5); 6.583(10.5);
6.569(10.3); 6.564(9.6); 4.920(1.1); 4.898(3.2); 4.882(4.4); 4.864(2.8); 4.860(3.1); 4.846(1.2); 4.843(1.0); 4.783(0.4); 4.193(1.9); 4.172(5.0); 4.153(5.9); 4.133(2.1); 3.902(7.5);
3.859(0.4); 3.836(0.4); 3.321(478.2); 3.175(0.3); 2.679(2.0); 2.675(4.1); 2.670(5.4); 2.666(3.9); 2.661(1.9); 2.541(3.4); 2.524(15.8); 2.510(338.7); 2.506(674.4); 2.501(874.5);
2.497(622.1); 2.492(293.6); 2.461(11.5); 2.453(5.4); 2.445(6.1); 2.439(4.4); 2.436(4.2); 2.429(2.6); 2.419(3.6); 2.402(1.4); 2.337(1.9); 2.333(3.9); 2.328(5.3); 2.323(3.9); 2.319(1.8);
2.276(0.4); 2.253(0.4); 2.222(1.7); 2.218(1.9); 2.200(3.8); 2.192(3.1); 2.185(3.2); 2.180(3.9); 2.173(2.4); 2.170(2.3); 2.166(2.6); 2.158(2.5); 2.136(1.0); 2.132(0.9); 2.114(0.5);
2.100(1.2); 2.090(1.5); 2.078(2.2); 2.066(4.1); 2.056(3.9); 2.036(2.7); 2.027(1.1); 2.016(1.3); 1.634(0.4); 1.613(0.4); 1.608(0.4); 1.236(0.6); 0.146(1.0); 0.008(8.4); 0.000(250.4); −0.009
(8.1); −0.150(1.0)
Example 251: ¹H-NMR(400.0 MHz. DMSO): δ = 8.399(4.5); 8.377(4.5); 8.315(1.8); 7.697(0.8); 7.686(4.5); 7.678(5.1); 7.673(5.3); 7.669(2.8); 7.663(6.2); 7.654(1.2); 7.565(1.9);
7.555(16.0); 7.546(10.1); 7.540(10.0); 7.532(14.5); 7.519(5.9); 7.515(5.7); 7.499(7.5); 7.496(7.3); 7.438(7.0); 7.435(7.6); 7.418(9.0); 7.415(10.2); 7.405(4.0); 7.402(3.8); 7.387(8.4);
7.384(6.9); 7.368(4.5); 7.365(3.6); 7.307(5.4); 7.303(5.3); 7.288(7.0); 7.284(6.6); 7.269(2.9); 7.265(2.7); 6.725(0.9); 6.715(4.9); 6.710(2.7); 6.706(5.2); 6.701(4.7); 6.693(4.6);
6.682(0.6); 4.938(0.8); 4.916(2.5); 4.900(3.4); 4.883(2.2); 4.878(2.3); 4.864(0.9); 4.860(0.8); 4.200(1.5); 4.179(3.8); 4.159(4.4); 4.139(1.6); 3.902(5.8); 3.321(338.7); 2.679(1.4);
2.675(2.9); 2.670(4.0); 2.666(2.8); 2.661(1.4); 2.541(2.6); 2.524(11.8); 2.510(249.5); 2.506(494.3); 2.501(637.2); 2.497(452.3); 2.492(212.7); 2.463(8.7); 2.455(4.1); 2.447(4.7);
2.440(3.3); 2.438(3.2); 2.430(1.9); 2.420(2.8); 2.403(1.1); 2.337(1.4); 2.333(2.8); 2.328(3.8); 2.323(2.7); 2.319(1.3); 2.230(1.1); 2.227(1.2); 2.222(1.2); 2.205(2.7); 2.197(2.1);
2.190(2.4); 2.184(3.0); 2.179(1.8); 2.175(1.7); 2.170(1.9); 2.163(1.8); 2.140(0.5); 2.061(0.8); 2.050(0.9); 2.039(1.7); 2.027(3.1); 2.016(2.9); 1.997(2.1); 1.988(0.9); 1.976(1.0);
1.235(0.6); 1.225(0.3); 0.146(0.7); 0.008(6.2); 0.000(181.4); −0.009(5.9); −0.150(0.7)
Example 252: ¹H-NMR(400.0 MHz. DMSO): δ = 8.591(4.9); 8.568(4.9); 8.315(1.6); 7.455(6.4); 7.451(6.7); 7.436(9.1); 7.432(8.7); 7.422(4.5); 7.417(3.9); 7.405(3.0); 7.401(8.2);
7.396(3.1); 7.384(5.0); 7.380(12.7); 7.377(9.7); 7.360(11.0); 7.357(11.4); 7.327(4.2); 7.324(4.3); 7.309(9.4); 7.305(8.4); 7.290(5.5); 7.287(4.6); 7.246(6.4); 7.242(6.3); 7.227(8.1);
7.223(7.8); 7.208(3.3); 7.204(3.1); 7.047(2.2); 7.040(13.0); 7.021(16.0); 7.019(15.4); 7.000(11.3); 6.992(1.8); 4.962(1.0); 4.953(1.2); 4.943(2.8); 4.930(3.0); 4.924(2.9); 4.912(3.1);
4.889(1.1); 4.169(1.8); 4.149(4.8); 4.128(4.5); 4.108(1.7); 3.902(6.0); 3.322(355.1); 2.675(2.8); 2.670(3.8); 2.666(2.8); 2.541(2.4); 2.524(10.9); 2.510(246.9); 2.506(488.3);
2.501(630.8); 2.497(448.4); 2.492(211.5); 2.471(4.3); 2.460(4.1); 2.454(4.3); 2.449(5.8); 2.443(9.2); 2.432(6.2); 2.424(5.6); 2.403(3.7); 2.387(1.5); 2.337(1.5); 2.333(2.9); 2.328(3.9);
2.323(2.8); 2.239(0.8); 2.215(2.5); 2.204(2.1); 2.195(3.0); 2.189(3.7); 2.184(3.2); 2.175(2.1); 2.170(2.8); 2.163(1.4); 2.156(0.9); 2.147(1.3); 2.033(1.4); 2.028(1.5); 2.024(1.5);
2.016(1.5); 2.006(2.5); 1.993(3.8); 1.983(2.9); 1.960(1.2); 1.948(0.6); 1.259(0.4); 1.235(0.8); 0.146(0.8); 0.008(7.1); 0.000(194.4); −0.009(6.1); −0.150(0.8)
Example 253: ¹H-NMR(400.0 MHz. DMSO): δ = 8.320(3.1); 8.306(7.6); 8.298(3.1); 7.548(0.6); 7.538(4.8); 7.534(3.2); 7.529(2.9); 7.524(3.3); 7.521(3.6); 7.516(5.6); 7.504(4.4);
7.501(4.1); 7.485(5.4); 7.481(5.2); 7.409(4.7); 7.406(5.1); 7.389(6.2); 7.386(7.0); 7.379(3.0); 7.376(2.7); 7.360(5.9); 7.357(4.9); 7.341(3.2); 7.338(2.6); 7.290(0.8); 7.285(1.9);
7.280(4.1); 7.275(4.7); 7.271(6.6); 7.267(9.9); 7.258(16.0); 7.249(9.9); 7.243(6.1); 7.238(2.1); 7.231(1.5); 7.225(0.7); 6.720(0.5); 6.711(5.2); 6.704(3.4); 6.702(3.3); 6.698(3.1);
6.693(3.3); 6.687(4.9); 6.679(0.4); 4.936(0.6); 4.915(1.8); 4.899(2.4); 4.881(1.6); 4.877(1.6); 4.863(0.6); 4.198(1.0); 4.177(2.8); 4.158(3.2); 4.138(1.2); 3.896(4.0); 3.327(503.2);
3.323(433.4); 2.677(2.1); 2.672(2.9); 2.668(2.1); 2.663(1.0); 2.542(1.7); 2.525(8.2); 2.512(183.5); 2.508(361.0); 2.503(467.3); 2.499(332.6); 2.494(157.5); 2.475(4.9); 2.459(6.6);
2.453(3.2); 2.443(3.6); 2.439(2.7); 2.436(2.5); 2.426(1.5); 2.418(2.1); 2.401(0.9); 2.339(1.1); 2.334(2.1); 2.330(2.9); 2.325(2.1); 2.223(0.9); 2.218(0.9); 2.200(2.0); 2.192(1.6);
2.185(1.8); 2.180(2.1); 2.174(1.3); 2.170(1.2); 2.166(1.3); 2.159(1.4); 2.133(0.5); 2.091(0.6); 2.080(0.8); 2.069(1.1); 2.058(2.3); 2.047(2.2); 2.028(1.6); 2.018(0.7); 2.006(0.7);
1.237(0.4); 0.146(0.5); 0.008(4.7); 0.000(123.9); −0.009(4.2); −0.150(0.5)
Example 254: ¹H-NMR(400.0 MHz. DMSO): δ = 8.708(11.0); 8.699(10.7); 8.697(10.6); 8.556(7.5); 8.533(7.5); 8.311(9.0); 7.662(9.0); 7.650(9.1); 7.643(10.0); 7.631(9.3); 7.496(8.7);
7.492(9.2); 7.477(12.6); 7.473(12.5); 7.457(0.5); 7.443(11.6); 7.440(12.3); 7.423(14.9); 7.420(16.0); 7.397(6.5); 7.394(6.5); 7.378(13.4); 7.375(11.8); 7.359(7.4); 7.356(6.2); 7.304(8.7);
7.300(8.7); 7.285(11.4); 7.281(10.9); 7.266(4.8); 7.262(4.4); 7.169(10.9); 7.167(11.3); 7.150(10.3); 7.147(10.1); 5.583(0.6); 4.946(1.8); 4.937(2.0); 4.926(4.2); 4.918(4.3); 4.904(4.1);
4.896(4.5); 4.886(1.8); 4.876(1.8); 4.203(2.6); 4.195(1.6); 4.183(6.4); 4.163(6.4); 4.150(1.5); 4.142(2.5); 4.097(0.5); 4.082(0.5); 3.900(13.3); 3.509(0.5); 3.485(0.4); 3.463(0.4);
3.325(953.3); 3.323(836.1); 3.267(0.5); 3.176(2.8); 3.163(2.7); 2.676(4.7); 2.671(6.2); 2.667(4.6); 2.662(2.2); 2.542(4.4); 2.524(19.2); 2.511(401.0); 2.507(784.8); 2.502(1015.5);
2.498(727.3); 2.493(348.0); 2.480(13.5); 2.475(14.6); 2.469(9.1); 2.460(12.2); 2.440(7.4); 2.422(3.1); 2.338(2.2); 2.333(4.4); 2.329(6.0); 2.324(4.4); 2.320(2.0); 2.257(0.6); 2.238(2.1);
2.232(2.6); 2.226(2.3); 2.214(3.6); 2.209(4.2); 2.204(4.5); 2.199(4.7); 2.189(2.7); 2.185(3.0); 2.177(2.4); 2.170(1.4); 2.162(1.8); 2.147(0.4); 2.038(0.5); 2.025(2.1); 2.019(3.5);
2.002(3.6); 1.997(3.9); 1.989(5.0); 1.973(3.1); 1.953(2.5); 1.947(1.3); 1.298(0.5); 1.259(0.6); 1.249(0.5); 1.236(1.0); 0.855(0.3); 0.146(1.0); 0.008(9.5); 0.000(244.6); −0.009(8.2); −0.150
(1.1)
Example 255: ¹H-NMR(400.0 MHz. DMSO): δ = 8.315(0.4); 7.951(3.9); 7.842(1.4); 7.821(1.4); 7.530(1.7); 7.513(2.1); 7.509(1.7); 7.328(3.9); 7.325(2.0); 7.308(5.0); 7.293(1.4);
7.212(1.5); 7.208(1.4); 7.194(1.3); 7.189(2.1); 7.174(0.8); 7.170(0.8); 7.154(1.5); 7.019(3.2); 6.883(1.6); 4.872(0.7); 4.859(0.8); 4.853(0.7); 4.840(0.7); 4.153(0.4); 4.133(1.1);
4.113(1.1); 4.094(0.4); 3.902(0.9); 3.843(16.0); 3.324(196.3); 3.302(0.6); 2.675(0.6); 2.671(0.8); 2.666(0.6); 2.541(0.5); 2.524(2.4); 2.510(54.0); 2.506(105.8); 2.502(137.2);
2.497(98.6); 2.493(47.4); 2.464(0.5); 2.447(0.7); 2.439(0.7); 2.424(1.7); 2.415(1.4); 2.409(2.1); 2.392(1.2); 2.376(0.5); 2.333(0.6); 2.328(0.8); 2.324(0.6); 2.226(0.5); 2.216(0.4);
2.211(0.5); 2.203(0.5); 2.191(0.8); 2.183(0.7); 2.174(0.5); 2.167(0.4); 2.162(0.5); 2.108(0.5); 2.090(0.6); 2.086(0.6); 2.075(1.0); 2.060(0.5); 2.045(0.4); 0.008(1.1); 0.000(30.2); −0.009
(1.1)
Example 256: ¹H-NMR(400.0 MHz. DMSO): δ = 8.887(3.5); 8.865(3.5); 8.315(1.0); 7.650(7.1); 7.629(8.8); 7.557(10.1); 7.552(10.9); 7.490(4.3); 7.469(12.7); 7.463(6.0); 7.453(3.6);
7.447(5.8); 7.444(6.2); 7.442(6.5); 7.431(5.4); 7.425(1.9); 7.420(2.4); 7.411(3.5); 7.398(2.4); 7.389(16.0); 7.380(6.6); 7.376(5.2); 7.372(4.1); 7.356(0.6); 7.354(0.6); 4.703(0.7);
4.681(2.2); 4.659(3.2); 4.638(2.3); 4.616(0.7); 3.902(3.2); 3.725(1.3); 3.702(3.1); 3.679(3.3); 3.656(1.4); 3.325(593.8); 2.675(1.9); 2.671(2.6); 2.666(1.8); 2.662(0.9); 2.541(1.5);
2.524(6.8); 2.510(158.8); 2.506(314.6); 2.502(407.0); 2.497(289.9); 2.493(137.2); 2.436(0.4); 2.333(1.8); 2.328(2.5); 2.324(1.8); 2.269(0.9); 2.247(3.4); 2.224(6.0); 2.202(4.0);
2.181(1.3); 2.082(0.7); 2.056(2.4); 2.032(3.4); 2.008(2.1); 1.984(0.7); 1.677(0.8); 1.651(2.3); 1.630(2.8); 1.625(2.7); 1.604(1.9); 1.578(0.6); 1.258(0.5); 1.235(0.4); 0.008(2.5);
0.000(76.9); −0.009(2.6); −0.150(0.3)
Example 257: ¹H-NMR(400.0 MHz. DMSO): δ = 9.095(3.6); 9.073(3.6); 8.545(5.9); 8.542(6.1); 8.534(6.1); 8.531(5.9); 8.315(2.0); 8.030(5.9); 8.026(5.8); 8.009(6.5); 8.006(6.2);
7.640(6.8); 7.619(8.5); 7.602(0.5); 7.580(0.7); 7.576(0.6); 7.571(0.7); 7.555(10.1); 7.550(16.0); 7.539(5.9); 7.530(5.5); 7.518(5.5); 7.471(6.2); 7.465(5.3); 7.450(4.7); 7.444(4.1);
6.527(0.7); 5.816(0.4); 5.805(0.4); 4.760(0.7); 4.737(2.4); 4.716(3.5); 4.694(2.5); 4.671(0.7); 3.902(6.5); 3.777(1.3); 3.753(3.3); 3.730(3.4); 3.707(1.5); 3.508(0.4); 3.445(0.4);
3.326(1449.2); 3.255(0.5); 3.175(0.4); 2.675(3.9); 2.671(5.2); 2.666(3.8); 2.617(0.4); 2.596(0.5); 2.578(0.4); 2.541(2.8); 2.506(673.9); 2.502(866.4); 2.497(624.6); 2.493(304.9);
2.395(0.5); 2.351(0.4); 2.333(4.0); 2.328(5.3); 2.324(3.9); 2.291(1.1); 2.268(2.8); 2.243(4.0); 2.222(3.9); 2.199(3.2); 2.177(1.4); 2.140(1.0); 2.115(2.7); 2.091(3.4); 2.067(2.1);
2.042(0.7); 1.652(0.9); 1.626(2.5); 1.605(2.8); 1.601(2.8); 1.579(2.2); 1.553(0.7); 1.259(0.4); 1.234(0.9); 0.000(52.0); −0.009(2.0)
Example 258: ¹H-NMR(400.0 MHz. DMSO): δ = 9.236(5.8); 9.214(5.8); 8.972(11.5); 8.969(11.3); 8.601(11.7); 8.598(11.1); 8.315(1.7); 7.640(10.2); 7.619(13.0); 7.564(14.6);
7.558(16.0); 7.507(0.4); 7.480(9.1); 7.475(7.9); 7.459(6.9); 7.454(6.1); 4.757(1.0); 4.734(3.4); 4.714(4.8); 4.691(3.4); 4.669(1.0); 3.902(6.3); 3.762(2.0); 3.740(4.8); 3.716(5.1);
3.693(2.0); 3.510(0.3); 3.325(1033.4); 3.279(0.6); 3.267(0.4); 2.675(3.2); 2.671(4.3); 2.666(3.2); 2.662(1.6); 2.541(2.8); 2.524(12.6); 2.511(271.0); 2.506(533.8); 2.502(693.9);
2.497(500.9); 2.493(241.3); 2.333(3.2); 2.329(4.3); 2.324(3.2); 2.299(1.4); 2.275(4.6); 2.252(8.5); 2.228(5.6); 2.206(1.9); 2.118(1.1); 2.092(3.6); 2.068(5.0); 2.045(3.0); 2.020(1.0);
1.690(1.1); 1.664(3.4); 1.643(4.0); 1.638(4.1); 1.618(2.9); 1.591(0.8); 1.236(0.4); 0.146(0.4); 0.008(3.8); 0.000(99.1); −0.008(3.6); −0.150(0.4)
Example 259: ¹H-NMR(400.0 MHz. DMSO): δ = 8.881(11.1); 8.878(10.7); 8.656(5.4); 8.634(5.4); 8.490(11.2); 8.487(10.5); 8.315(1.7); 7.558(0.4); 7.524(15.4); 7.519(16.0);
7.467(8.1); 7.446(15.0); 7.402(10.3); 7.396(9.2); 7.381(5.7); 7.375(5.2); 4.963(0.8); 4.957(0.9); 4.945(2.2); 4.940(2.0); 4.926(3.2); 4.909(2.5); 4.889(0.8); 4.179(1.7); 4.159(4.8);
4.138(4.1); 4.119(1.5); 3.902(6.3); 3.423(0.4); 3.405(0.4); 3.327(1112.1); 3.263(0.4); 3.252(0.4); 2.680(1.6); 2.675(3.2); 2.671(4.3); 2.666(3.1); 2.662(1.4); 2.541(2.9); 2.524(12.5);
2.511(273.8); 2.506(539.1); 2.502(694.3); 2.497(491.6); 2.493(229.8); 2.472(7.9); 2.467(5.9); 2.454(3.3); 2.448(3.9); 2.426(2.8); 2.408(1.2); 2.338(1.6); 2.333(3.2); 2.329(4.3);
2.324(3.1); 2.320(1.4); 2.257(0.7); 2.251(0.7); 2.235(1.5); 2.229(2.3); 2.224(2.1); 2.214(2.8); 2.208(3.1); 2.202(2.3); 2.193(2.0); 2.187(2.4); 2.166(1.0); 2.161(0.8); 2.035(1.4);
2.013(2.3); 2.007(1.9); 1.994(2.8); 1.984(2.8); 1.973(1.8); 1.961(0.5); 1.952(0.7); 1.236(0.3); 0.850(0.4); 0.008(2.6); 0.000(73.2); −0.009(2.3)
Example 260: ¹H-NMR(400.0 MHz. DMSO): δ = 8.530(0.4); 8.488(4.0); 8.466(4.0); 8.440(7.9); 8.436(8.3); 8.428(8.3); 8.425(8.0); 8.314(1.2); 7.997(0.3); 7.918(7.8); 7.915(7.9);
7.898(8.8); 7.894(8.4); 7.594(0.5); 7.589(0.5); 7.554(0.5); 7.532(0.6); 7.521(0.5); 7.499(14.4); 7.494(16.0); 7.490(9.1); 7.469(12.5); 7.460(8.6); 7.449(8.1); 7.440(7.5); 7.428(7.4);
7.392(8.0); 7.387(7.2); 7.371(5.5); 7.366(5.0); 4.924(1.0); 4.912(2.1); 4.904(2.3); 4.894(2.1); 4.882(2.2); 4.859(0.9); 4.172(1.4); 4.151(3.6); 4.131(3.4); 4.111(1.3); 3.902(6.4);
3.474(0.4); 3.461(0.5); 3.447(0.4); 3.415(0.7); 3.393(1.1); 3.376(1.4); 3.330(934.6); 3.304(3.2); 3.287(1.2); 3.271(0.9); 3.256(0.5); 3.240(0.4); 3.230(0.4); 2.676(2.1); 2.671(2.7);
2.667(2.0); 2.636(0.4); 2.574(0.6); 2.565(0.6); 2.541(2.2); 2.511(180.2); 2.507(345.4); 2.502(441.9); 2.498(316.2); 2.493(153.0); 2.470(5.3); 2.463(7.2); 2.452(5.3); 2.445(4.4);
2.425(2.3); 2.408(1.0); 2.338(0.9); 2.333(1.9); 2.329(2.6); 2.324(1.8); 2.244(0.6); 2.221(1.8); 2.208(1.5); 2.201(2.3); 2.194(2.7); 2.189(2.2); 2.180(1.8); 2.175(2.0); 2.160(0.7);
2.152(1.0); 2.077(1.3); 2.054(1.8); 2.044(2.7); 2.032(2.3); 2.008(0.9); 1.336(0.4); 1.274(0.4); 1.259(0.8); 1.243(0.7); 1.235(1.0); 1.225(0.5); 1.211(0.4); 1.195(0.4); 0.867(0.5);
0.849(1.0); 0.830(0.5); 0.008(0.5); 0.000(11.1); −0.009(0.4)
Example 261: ¹H-NMR(400.0 MHz. DMSO): δ = 8.375(4.9); 8.354(4.8); 8.314(1.3); 7.650(0.4); 7.629(0.5); 7.547(15.4); 7.542(16.0); 7.498(7.6); 7.477(15.9); 7.445(11.0); 7.440(9.7);
7.424(5.2); 7.419(4.8); 7.402(3.1); 7.398(4.4); 7.382(12.1); 7.378(13.3); 7.372(6.8); 7.358(7.9); 7.354(7.6); 7.338(3.3); 7.334(3.2); 7.297(5.2); 7.293(4.9); 7.278(7.2); 7.275(7.0);
7.261(3.7); 7.257(3.5); 6.929(7.9); 6.926(7.5); 6.911(7.5); 6.907(6.9); 4.916(1.2); 4.906(2.6); 4.896(2.8); 4.891(2.6); 4.885(2.6); 4.875(2.7); 4.854(1.1); 4.153(1.6); 4.134(4.1);
4.113(3.9); 4.094(1.6); 3.902(4.4); 3.433(0.4); 3.418(0.6); 3.400(0.6); 3.385(0.9); 3.328(879.0); 3.283(0.9); 3.264(0.8); 3.246(0.4); 3.170(0.6); 2.675(2.1); 2.671(2.8); 2.666(2.0);
2.591(0.4); 2.578(0.4); 2.568(0.4); 2.541(2.4); 2.511(196.5); 2.506(369.0); 2.502(466.8); 2.497(334.0); 2.493(161.9); 2.452(3.8); 2.435(6.7); 2.430(6.6); 2.420(6.8); 2.414(5.6);
2.403(3.0); 2.396(3.2); 2.386(1.5); 2.379(1.4); 2.333(2.1); 2.329(2.7); 2.324(2.1); 2.247(0.3); 2.235(0.6); 2.212(2.1); 2.190(2.8); 2.182(3.1); 2.170(2.0); 2.165(2.1); 2.151(1.0);
2.141(1.4); 2.070(1.3); 2.062(1.6); 2.031(3.6); 2.023(2.3); 2.015(2.1); 1.998(1.3); 1.236(0.5); 1.154(0.4); 0.000(9.5)
Example 262: ¹H-NMR(400.0 MHz. DMSO): δ = 8.580(1.4); 8.559(1.5); 8.315(0.3); 8.254(3.7); 7.552(1.6); 7.549(1.6); 7.533(1.9); 7.530(1.9); 7.442(1.4); 7.394(2.0); 7.391(2.1);
7.374(2.5); 7.371(2.7); 7.354(1.0); 7.351(1.0); 7.335(2.2); 7.333(2.0); 7.317(1.4); 7.313(1.4); 7.306(3.3); 7.252(1.4); 7.248(1.4); 7.233(1.9); 7.229(1.8); 7.214(0.8); 7.210(0.8);
7.171(1.6); 4.709(0.8); 4.688(1.3); 4.666(0.9); 3.906(16.0); 3.790(0.5); 3.767(1.2); 3.743(1.3); 3.720(0.5); 3.330(173.9); 2.676(0.5); 2.671(0.7); 2.667(0.5); 2.542(0.6); 2.524(2.0);
2.511(46.4); 2.507(91.1); 2.502(117.4); 2.498(84.8); 2.493(41.6); 2.334(0.5); 2.329(0.7); 2.324(0.5); 2.291(0.4); 2.268(1.0); 2.244(1.1); 2.233(0.5); 2.220(0.6); 2.209(1.0); 2.188(1.1);
2.167(0.5); 2.066(0.4); 2.041(1.0); 2.017(1.3); 1.993(0.8); 1.637(0.4); 1.611(1.0); 1.590(1.1); 1.585(1.1); 1.564(0.9); 1.235(0.4); 0.842(0.4); 0.000(3.8)
Example 263: ¹H-NMR(400.0 MHz. DMSO): δ = 8.309(0.9); 8.062(3.9); 8.014(1.3); 7.993(1.3); 7.752(1.6); 7.732(2.0); 7.615(1.1); 7.597(3.9); 7.578(3.5); 7.373(1.1); 7.354(1.9);
7.335(0.8); 7.030(1.4); 6.894(2.9); 6.759(1.5); 4.768(0.8); 4.761(0.8); 4.748(0.8); 4.742(0.7); 4.142(0.4); 4.122(0.9); 4.103(0.9); 3.899(0.8); 3.867(16.0); 3.330(285.4); 3.266(0.4);
3.177(0.5); 3.164(0.5); 2.677(0.6); 2.672(0.8); 2.668(0.6); 2.542(0.8); 2.512(55.7); 2.508(102.8); 2.503(129.6); 2.499(93.5); 2.494(46.6); 2.480(2.9); 2.461(1.1); 2.442(0.5); 2.334(0.6);
2.330(0.8); 2.325(0.6); 2.242(0.5); 2.227(0.5); 2.220(0.8); 2.213(0.8); 2.200(0.6); 2.194(0.6); 2.172(0.3); 2.135(0.5); 2.113(0.7); 2.104(0.9); 2.093(0.7); 0.000(1.4)
Example 264: ¹H-NMR(400.0 MHz. DMSO): δ = 8.826(5.6); 8.804(5.7); 8.315(1.0); 7.869(8.9); 7.851(9.5); 7.828(0.4); 7.685(9.8); 7.664(11.9); 7.547(14.8); 7.541(16.0); 7.505(0.3);
7.466(8.4); 7.461(7.7); 7.446(8.6); 7.440(6.9); 7.431(9.1); 7.429(8.9); 7.413(5.7); 7.410(5.4); 7.327(0.3); 7.303(8.4); 7.300(9.0); 7.284(6.9); 7.281(6.5); 7.178(4.7); 7.174(4.5);
7.158(7.4); 7.155(7.1); 7.139(4.0); 7.135(3.6); 4.714(1.0); 4.692(3.4); 4.669(5.0); 4.648(3.5); 4.626(1.0); 3.902(3.9); 3.721(1.8); 3.698(4.9); 3.674(4.9); 3.651(1.9); 3.449(0.5);
3.431(0.4); 3.411(0.7); 3.397(0.8); 3.384(0.9); 3.367(2.4); 3.327(967.7); 3.279(1.4); 3.265(0.9); 3.250(0.4); 3.231(0.3); 3.197(0.4); 2.675(2.4); 2.671(3.2); 2.666(2.4); 2.618(0.4);
2.592(0.4); 2.583(0.5); 2.541(3.1); 2.510(228.1); 2.506(432.4); 2.502(549.6); 2.497(399.2); 2.493(198.1); 2.444(1.3); 2.439(1.1); 2.419(0.5); 2.407(0.4); 2.365(0.4); 2.333(2.5);
2.329(3.3); 2.324(2.5); 2.298(1.6); 2.275(4.0); 2.249(5.0); 2.228(5.1); 2.202(4.3); 2.181(1.9); 2.096(1.2); 2.071(3.8); 2.047(5.0); 2.023(3.1); 1.998(1.0); 1.650(1.2); 1.623(3.6);
1.602(4.0); 1.597(4.0); 1.577(3.2); 1.551(0.9); 1.235(0.5); 0.000(10.5)
Example 265: ¹H-NMR(400.0 MHz. DMSO): δ = 8.945(4.7); 8.923(4.7); 8.313(1.4); 7.674(12.0); 7.670(11.9); 7.636(8.3); 7.615(10.2); 7.554(11.6); 7.549(12.1); 7.525(0.4);
7.519(0.4); 7.494(4.9); 7.489(4.4); 7.473(9.8); 7.468(10.6); 7.465(8.2); 7.459(6.4); 7.444(6.0); 7.434(16.0); 7.413(7.7); 4.681(0.8); 4.660(2.7); 4.638(4.0); 4.617(2.7); 4.596(0.8);
3.901(2.8); 3.723(1.5); 3.700(3.9); 3.677(4.1); 3.653(1.7); 3.454(0.5); 3.436(0.5); 3.426(0.5); 3.408(0.6); 3.329(830.7); 3.275(0.9); 3.265(0.9); 3.175(0.4); 2.676(1.9); 2.671(2.5);
2.667(1.8); 2.577(0.4); 2.541(2.6); 2.507(330.4); 2.502(416.5); 2.498(301.8); 2.456(0.8); 2.439(0.8); 2.334(1.9); 2.329(2.4); 2.324(1.9); 2.270(1.2); 2.247(4.3); 2.225(7.1); 2.204(4.8);
2.183(1.6); 2.073(0.9); 2.048(3.0); 2.024(4.1); 2.000(2.6); 1.975(0.8); 1.688(0.9); 1.662(2.7); 1.641(3.4); 1.637(3.3); 1.615(2.3); 1.589(0.7); 1.236(0.5); 0.000(7.7); −0.008(0.4)
Example 266: ¹H-NMR(400.0 MHz. DMSO): δ = 8.426(5.3); 8.404(5.3); 8.312(2.3); 7.668(0.8); 7.636(0.5); 7.614(0.7); 7.580(13.3); 7.575(13.2); 7.547(1.0); 7.535(13.1); 7.530(13.6);
7.516(0.3); 7.487(7.2); 7.466(16.0); 7.437(10.5); 7.432(9.6); 7.416(5.0); 7.410(10.1); 7.405(7.2); 7.389(8.2); 7.384(7.7); 6.996(14.9); 6.975(13.7); 4.895(2.5); 4.883(2.9); 4.863(2.7);
4.842(1.0); 4.147(1.7); 4.127(4.2); 4.107(4.0); 4.086(1.7); 3.901(4.5); 3.678(0.3); 3.516(0.4); 3.502(0.4); 3.492(0.4); 3.482(0.4); 3.466(0.6); 3.459(0.5); 3.448(0.4); 3.410(0.8);
3.387(2.2); 3.330(1115.8); 3.282(1.3); 3.260(0.8); 3.226(0.7); 3.206(0.4); 3.175(1.5); 3.163(1.4); 2.676(2.3); 2.672(3.0); 2.667(2.3); 2.639(0.4); 2.559(1.2); 2.542(2.8); 2.507(404.8);
2.502(515.8); 2.498(376.5); 2.459(2.8); 2.448(3.0); 2.436(4.8); 2.430(7.3); 2.420(5.9); 2.412(4.8); 2.391(3.2); 2.374(1.2); 2.334(2.3); 2.329(3.0); 2.325(2.3); 2.236(0.7); 2.212(2.1);
2.200(2.0); 2.192(2.7); 2.186(3.2); 2.181(2.8); 2.166(2.3); 2.152(0.7); 2.143(1.0); 2.047(1.5); 2.026(2.4); 2.012(3.4); 2.001(2.6); 1.979(1.0); 1.238(0.4); 0.851(0.6); 0.832(0.3);
0.000(4.9)
Example 267: ¹H-NMR(400.0 MHz. DMSO): δ = 9.113(5.0); 9.091(5.0); 8.315(0.8); 7.875(6.8); 7.855(8.5); 7.728(4.1); 7.709(7.5); 7.690(3.8); 7.673(7.4); 7.654(8.9); 7.527(1.6);
7.510(3.8); 7.506(3.5); 7.489(6.9); 7.473(3.7); 7.468(4.3); 7.456(5.0); 7.438(7.5); 7.419(3.4); 7.160(2.2); 7.153(11.6); 7.133(16.0); 7.113(9.7); 7.105(1.7); 4.823(1.0); 4.802(3.7);
4.780(5.4); 4.758(3.8); 4.737(1.0); 3.902(3.6); 3.742(1.4); 3.719(4.0); 3.696(3.9); 3.673(1.5); 3.458(0.6); 3.439(0.4); 3.428(0.5); 3.398(1.1); 3.370(2.0); 3.331(1043.0); 3.271(0.8);
3.260(0.4); 3.216(0.4); 2.676(2.0); 2.671(2.7); 2.667(2.0); 2.575(0.5); 2.541(2.3); 2.524(8.1); 2.511(188.9); 2.507(364.2); 2.502(462.9); 2.498(333.9); 2.493(163.3); 2.441(0.6);
2.393(0.4); 2.333(2.1); 2.329(2.8); 2.325(2.0); 2.288(1.5); 2.267(4.1); 2.245(4.4); 2.224(1.9); 2.179(1.4); 2.156(3.7); 2.133(4.4); 2.109(2.0); 2.053(1.7); 2.028(4.1); 2.004(5.5);
1.981(3.3); 1.956(1.2); 1.782(1.3); 1.756(3.8); 1.734(4.2); 1.731(4.2); 1.709(3.1); 1.684(0.9); 1.234(0.4); 0.000(4.5)
Example 268: ¹H-NMR(400.0 MHz. DMSO): δ = 8.669(4.1); 8.304(1.8); 8.298(1.0); 7.473(0.8); 7.456(2.0); 7.452(2.3); 7.435(3.8); 7.415(2.6); 7.398(1.0); 7.279(7.3); 7.258(13.2);
7.207(16.0); 7.186(9.2); 7.087(4.3); 7.079(2.8); 7.068(7.4); 7.047(3.8); 3.895(0.8); 3.461(0.3); 3.333(385.0); 3.329(422.9); 3.269(0.8); 3.250(0.6); 3.188(1.1); 3.176(2.1); 3.169(2.4);
3.158(3.7); 3.147(2.5); 3.139(2.1); 3.127(1.0); 2.673(1.6); 2.543(2.1); 2.508(215.5); 2.504(267.8); 2.500(201.5); 2.436(0.4); 2.418(0.4); 2.331(1.6); 2.312(1.6); 2.294(3.5); 2.272(3.8);
2.254(1.7); 1.350(1.8); 1.335(2.7); 1.330(3.3); 1.314(3.6); 1.293(1.9); 1.238(0.4); 1.167(2.1); 1.153(4.0); 1.138(3.9); 1.123(1.8); 0.000(6.2)
Example 269: ¹H-NMR(400.0 MHz. DMSO): δ = 8.306(3.5); 8.297(3.5); 7.476(1.0); 7.467(1.3); 7.462(1.8); 7.454(2.8); 7.448(2.3); 7.440(2.7); 7.432(2.6); 7.419(1.3); 7.298(8.5);
7.276(16.0); 7.230(15.2); 7.224(6.6); 7.208(8.4); 7.197(5.1); 7.187(7.9); 7.178(8.6); 7.175(8.9); 7.166(7.5); 3.902(2.1); 3.436(0.4); 3.398(0.5); 3.330(530.3); 3.288(0.8); 3.272(0.7);
3.176(1.0); 3.164(1.8); 3.157(2.1); 3.146(3.1); 3.136(2.1); 3.128(1.7); 3.115(0.9); 2.671(1.5); 2.541(1.8); 2.506(210.9); 2.502(251.2); 2.498(180.7); 2.445(0.4); 2.329(1.5); 2.319(1.8);
2.300(3.4); 2.278(3.3); 2.261(1.5); 1.347(1.5); 1.332(2.6); 1.327(2.7); 1.311(3.5); 1.290(1.9); 1.271(2.2); 1.257(3.7); 1.241(3.6); 1.226(1.4); 0.000(2.6)
Example 270: ¹H-NMR(400.0 MHz. DMSO): δ = 8.688(3.8); 8.678(3.8); 8.314(0.5); 7.648(0.9); 7.629(2.3); 7.614(2.8); 7.609(2.8); 7.594(2.6); 7.568(4.5); 7.561(7.7); 7.545(5.9);
7.526(1.9); 7.286(9.3); 7.265(16.0); 7.206(14.8); 7.185(8.8); 3.902(1.1); 3.427(0.4); 3.402(0.7); 3.332(745.7); 3.290(1.3); 3.270(1.0); 3.246(0.5); 3.205(0.4); 3.198(0.9); 3.185(1.8);
3.176(2.1); 3.166(3.4); 3.155(2.2); 3.147(1.9); 3.135(1.0); 2.671(1.7); 2.541(1.8); 2.506(218.0); 2.502(278.7); 2.498(207.7); 2.333(1.3); 2.329(1.6); 2.311(1.5); 2.291(3.4); 2.270(3.5);
2.251(1.6); 1.353(1.7); 1.338(2.4); 1.332(3.0); 1.317(3.2); 1.311(2.2); 1.296(1.8); 1.162(1.9); 1.147(3.5); 1.132(3.4); 1.117(1.6); 0.000(1.1)
Example 271: ¹H-NMR(400.0 MHz. DMSO): δ = 8.497(4.5); 8.487(4.3); 8.314(0.7); 7.718(3.8); 7.702(4.9); 7.697(4.7); 7.683(0.4); 7.619(1.4); 7.604(4.9); 7.601(4.8); 7.589(9.4);
7.583(4.6); 7.575(4.0); 7.557(1.1); 7.346(11.1); 7.325(16.0); 7.236(15.0); 7.215(10.3); 6.846(4.1); 6.841(4.4); 6.825(4.1); 3.902(15.9); 3.451(0.5); 3.432(0.6); 3.421(0.7); 3.389(1.3);
3.331(812.5); 3.283(1.4); 3.267(0.8); 3.250(0.6); 3.155(1.1); 3.143(2.1); 3.135(2.4); 3.124(3.8); 3.114(2.5); 3.106(2.1); 3.094(1.1); 2.676(1.5); 2.671(2.0); 2.667(1.5); 2.541(1.9);
2.506(261.2); 2.502(320.9); 2.498(236.2); 2.329(3.5); 2.311(3.8); 2.288(3.7); 2.270(1.7); 1.336(1.8); 1.321(2.9); 1.315(3.0); 1.300(3.8); 1.279(2.1); 1.246(2.4); 1.229(4.0); 1.216(3.9);
1.201(1.7); 1.176(0.5); 0.000(2.5)
Example 272: ¹H-NMR(400.0 MHz. DMSO): δ = 8.406(3.4); 8.396(3.4); 7.577(4.3); 7.574(3.8); 7.558(5.2); 7.554(4.7); 7.363(0.3); 7.343(1.9); 7.340(2.3); 7.330(9.4); 7.325(8.0);
7.321(6.1); 7.308(16.0); 7.302(9.0); 7.297(4.8); 7.283(4.1); 7.278(4.1); 7.264(1.7); 7.259(1.4); 7.236(12.4); 7.215(7.9); 6.839(4.3); 6.834(4.5); 6.821(3.7); 6.816(3.8); 3.322(40.4);
3.158(0.8); 3.146(1.6); 3.138(1.8); 3.127(2.9); 3.116(1.9); 3.108(1.7); 3.096(0.9); 2.675(0.6); 2.670(0.8); 2.666(0.6); 2.506(103.0); 2.501(128.9); 2.497(95.1); 2.324(1.9); 2.306(3.0);
2.284(3.0); 2.266(1.3); 1.325(1.4); 1.310(2.3); 1.305(2.4); 1.288(3.0); 1.283(1.9); 1.267(1.7); 1.236(1.9); 1.221(3.1); 1.206(3.0); 1.191(1.3); 0.000(46.5)
Example 273: ¹H-NMR(400.0 MHz. DMSO): δ = 8.367(3.7); 8.357(3.7); 7.815(5.5); 7.797(5.7); 7.796(5.7); 7.347(2.8); 7.345(3.1); 7.334(10.1); 7.329(8.6); 7.313(16.0); 7.307(5.4);
7.241(13.9); 7.220(8.9); 7.117(2.9); 7.114(3.1); 7.098(4.6); 7.094(4.8); 7.079(2.3); 7.075(2.3); 6.737(4.8); 6.733(4.8); 6.718(4.6); 6.714(4.4); 5.756(7.2); 3.323(34.4); 3.152(0.8);
3.140(1.7); 3.132(1.9); 3.121(3.1); 3.111(1.9); 3.103(1.8); 3.091(0.9); 2.675(0.5); 2.670(0.6); 2.666(0.5); 2.541(0.4); 2.506(75.7); 2.501(97.2); 2.497(70.4); 2.328(0.7); 2.319(1.6);
2.300(3.1); 2.279(3.2); 2.261(1.5); 1.333(1.5); 1.318(2.4); 1.312(2.6); 1.296(3.2); 1.291(2.0); 1.275(1.8); 1.232(2.0); 1.217(3.3); 1.203(3.2); 1.187(1.5); 0.008(1.5); 0.000(37.5); −0.008
(1.4)
Example 274: ¹H-NMR(400.0 MHz. DMSO): δ = 8.568(3.8); 8.559(3.7); 8.408(5.0); 8.403(5.1); 8.396(5.5); 8.391(5.0); 8.315(0.6); 7.430(3.0); 7.418(2.8); 7.411(7.2); 7.400(7.5);
7.387(7.4); 7.382(7.6); 7.368(3.2); 7.363(2.7); 7.355(0.4); 7.346(0.3); 7.329(10.5); 7.308(16.0); 7.231(14.6); 7.210(9.6); 3.902(3.2); 3.323(235.3); 3.172(0.9); 3.161(1.8); 3.152(2.0);
3.142(3.3); 3.131(2.1); 3.123(1.9); 3.111(1.0); 2.671(1.7); 2.666(1.3); 2.541(1.5); 2.506(224.9); 2.502(282.2); 2.497(203.9); 2.349(1.5); 2.331(4.4); 2.309(3.4); 2.291(1.5); 1.351(1.7);
1.336(2.6); 1.330(2.8); 1.315(3.2); 1.309(2.2); 1.293(1.9); 1.235(0.4); 1.219(2.0); 1.204(3.5); 1.191(3.4); 1.175(1.6); 0.000(7.0)
Example 275: ¹H-NMR(400.0 MHz. DMSO): δ = 8.411(3.4); 8.401(3.4); 8.315(0.4); 7.416(2.3); 7.413(2.6); 7.396(6.8); 7.393(6.6); 7.384(3.6); 7.381(3.3); 7.367(4.4); 7.363(4.2);
7.347(2.0); 7.343(1.9); 7.322(8.4); 7.301(16.0); 7.282(4.6); 7.279(4.2); 7.264(2.2); 7.261(2.0); 7.233(12.4); 7.212(7.8); 6.908(4.6); 6.905(4.3); 6.890(4.2); 6.886(3.9); 3.902(1.7);
3.324(199.4); 3.162(0.9); 3.150(1.7); 3.143(1.8); 3.132(2.9); 3.121(1.9); 3.113(1.7); 3.102(0.8); 2.675(1.1); 2.671(1.3); 2.540(1.7); 2.506(184.3); 2.501(225.0); 2.497(167.8); 2.447(0.5);
2.437(0.6); 2.324(2.2); 2.307(3.1); 2.285(3.1); 2.267(1.4); 1.323(1.3); 1.308(2.3); 1.303(2.4); 1.288(3.1); 1.266(1.7); 1.240(1.9); 1.225(3.3); 1.211(3.2); 1.195(1.3); 0.000(3.9)
Example 276: ¹H-NMR(400.0 MHz. DMSO): δ = 8.240(1.5); 8.231(1.5); 7.311(0.5); 7.304(4.2); 7.300(1.5); 7.288(1.9); 7.283(7.1); 7.277(1.0); 7.258(0.8); 7.255(0.8); 7.239(2.0);
7.236(1.9); 7.228(1.3); 7.222(7.2); 7.218(3.3); 7.201(3.8); 7.141(1.4); 7.131(2.5); 7.123(2.3); 7.113(1.7); 7.106(1.2); 6.962(1.9); 6.958(2.0); 6.952(0.5); 6.942(1.8); 3.322(10.9);
3.158(0.4); 3.145(0.7); 3.139(0.8); 3.134(0.7); 3.127(1.3); 3.120(0.6); 3.115(0.9); 3.109(0.7); 3.097(0.4); 2.523(0.8); 2.510(16.2); 2.506(31.9); 2.501(41.6); 2.496(29.8); 2.492(14.2);
2.331(0.4); 2.315(0.6); 2.297(1.4); 2.276(1.4); 2.257(0.6); 1.895(16.0); 1.305(0.5); 1.290(1.3); 1.286(0.8); 1.271(2.5); 1.258(1.7); 1.254(1.7); 1.249(1.4); 1.241(1.5); 0.000(7.6)
Example 277: ¹H-NMR(299.9 MHz, cdcl3):
δ = 7.589(0.7); 7.413(3.7); 7.295(3.0); 7.288(1.6); 7.261(39.2); 7.249(3.1); 7.218(1.7); 7.189(0.7); 7.165(4.2); 7.137(2.8); 5.096(0.6); 5.071(0.7); 4.688(0.9); 4.663(1.2); 4.639(0.9); 3.795(4.9); 3.765(16.0);
3.733(0.4); 3.472(0.4); 3.448(1.0); 3.424(1.0); 3.400(0.4); 2.878(0.3); 2.409(0.5); 2.384(0.4); 2.366(4.8); 2.279(0.4); 2.249(0.9); 2.223(1.1); 2.218(1.1); 2.191(1.3); 2.171(0.8); 2.164(0.8); 2.149(0.6);
2.137(0.5); 2.081(15.6); 1.990(0.8); 1.979(0.8); 1.966(1.2); 1.955(1.4); 1.950(1.3); 1.928(1.0); 1.917(1.0); 1.898(0.6); 1.890(0.8); 1.866(0.8); 1.860(0.7); 1.850(0.5); 1.835(0.7); 1.823(0.7); 1.807(0.6);
1.793(0.8); 1.776(0.5); 1.765(0.5); 1.749(0.3); 1.737(0.3); 1.720(0.6); 1.693(0.8); 1.679(0.5); 1.666(0.7); 1.651(0.7); 1.638(0.5); 1.624(0.6); 1.610(0.5); 1.594(0.6); 1.559(20.5); 1.255(0.7); 0.010(2.5);
0.000(42.0); −0.011(2.5)
Example 278: ¹H-NMR(399.8 MHz, cdcl3):
δ = 7.575(1.0); 7.572(1.0); 7.570(0.9); 7.557(1.5); 7.552(1.3); 7.520(1.0); 7.359(0.9); 7.345(2.2); 7.340(6.8); 7.338(11.3); 7.335(6.4); 7.325(3.4); 7.320(10.1); 7.318(9.7); 7.316(10.0); 7.306(3.7);
7.297(18.7); 7.294(20.3); 7.288(18.3); 7.286(16.7); 7.283(31.9); 7.280(15.5); 7.266(19.7); 7.261(183.3); 7.247(11.7); 7.239(6.9); 7.234(6.1); 7.229(8.3); 7.225(5.5); 7.221(5.4); 7.214(4.8); 7.207(7.6);
7.202(26.8); 7.198(7.5); 7.185(6.1); 7.181(16.0); 6.997(0.8); 6.143(0.5); 6.122(0.5); 5.760(2.6); 5.742(2.6); 4.799(1.7); 4.782(4.7); 4.762(5.5); 4.745(4.0); 4.729(1.2); 4.530(0.7); 4.510(0.9); 4.487(0.8);
3.497(2.4); 3.478(6.3); 3.460(6.0); 3.441(2.2); 2.994(0.4); 2.970(0.9); 2.950(0.9); 2.926(0.5); 2.434(0.5); 2.420(0.8); 2.414(0.4); 2.401(0.8); 2.387(0.6); 2.292(0.6); 2.270(3.0); 2.248(7.3); 2.244(5.5);
2.237(5.4); 2.227(7.6); 2.217(4.0); 2.214(3.9); 2.205(3.1); 2.197(3.7); 2.178(0.7); 2.169(0.4); 2.159(0.4); 2.015(0.7); 1.994(2.1); 1.984(2.3); 1.972(4.1); 1.966(6.0); 1.962(5.3); 1.958(5.6); 1.955(5.7);
1.946(8.2); 1.935(3.8); 1.926(2.8); 1.915(4.1); 1.904(1.4); 1.895(2.4); 1.879(1.4); 1.874(2.3); 1.866(1.3); 1.855(1.9); 1.846(3.4); 1.838(2.3); 1.836(2.2); 1.827(2.6); 1.817(4.2); 1.809(4.7); 1.804(3.7);
1.800(4.1); 1.795(5.1); 1.790(5.2); 1.781(3.3); 1.776(3.6); 1.765(3.0); 1.761(3.2); 1.744(2.5); 1.733(0.8); 1.723(1.3); 1.714(0.5); 1.708(0.4); 1.694(0.8); 1.681(0.4); 1.672(0.7); 1.662(0.6); 1.651(0.4);
1.639(0.5); 1.554(70.4); 1.252(0.3); 0.146(0.7); 0.050(0.6); 0.008(7.0); 0.000(177.5); −0.009(5.6); −0.150(0.8)
Example 279: ¹H-NMR(399.8 MHz, cdcl3):
δ = 7.670(0.3); 7.647(0.5); 7.629(3.4); 7.624(2.3); 7.617(4.0); 7.606(4.3); 7.547(0.4); 7.521(1.1); 7.501(0.5); 7.487(1.3); 7.474(9.2); 7.463(8.5); 7.462(8.5); 7.452(8.3); 7.405(0.4); 7.385(0.4); 7.318(0.4);
7.311(0.7); 7.299(11.7); 7.294(4.6); 7.278(17.8); 7.273(5.4); 7.271(6.7); 7.264(78.5); 7.262(132.2); 7.254(3.6); 7.253(3.0); 7.252(2.5); 7.251(2.2); 7.249(2.0); 7.248(1.8); 7.247(1.7); 7.246(1.6); 7.244(1.5);
7.243(1.5); 7.238(2.1); 7.233(1.1); 7.232(1.1); 7.230(0.8); 7.217(1.2); 7.211(0.9); 7.192(16.0); 7.171(11.2); 7.116(0.4); 7.098(0.4); 6.998(0.9); 6.977(3.9); 6.968(3.8); 6.966(3.8); 6.961(2.5); 6.955(3.7);
5.285(2.1); 5.263(2.2); 4.801(1.1); 4.784(3.3); 4.766(3.8); 4.746(2.6); 4.729(1.0); 3.491(1.9); 3.472(4.6); 3.453(4.3); 3.435(1.5); 2.289(0.3); 2.265(2.1); 2.245(4.5); 2.237(3.6); 2.221(5.2); 2.204(2.7);
1.995(0.6); 1.982(1.1); 1.973(1.9); 1.960(1.4); 1.952(2.9); 1.943(3.8); 1.933(4.2); 1.919(2.7); 1.913(3.3); 1.901(2.7); 1.893(1.8); 1.881(2.9); 1.866(2.1); 1.846(2.5); 1.839(1.6); 1.828(1.7); 1.819(2.2);
1.808(2.2); 1.798(2.0); 1.783(2.8); 1.771(2.7); 1.756(2.4); 1.751(2.2); 1.736(2.3); 1.719(2.0); 1.699(0.9); 1.675(0.3); 1.599(0.4); 1.550(63.8); 1.285(0.4); 1.255(1.3); 0.835(0.4); 0.147(0.7); 0.050(0.4);
0.042(0.4); 0.037(0.5); 0.036(0.5); 0.035(0.6); 0.033(0.6); 0.032(0.6); 0.028(0.8); 0.027(0.9); 0.026(0.9); 0.025(1.0); 0.024(1.0); 0.022(1.1); 0.021(1.3); 0.020(1.4); 0.019(1.5); 0.017(1.8); 0.016(2.1);
0.015(2.3); 0.014(2.9); 0.010(6.5); 0.008(9.8); 0.002(92.6); 0.000(153.2); −0.005(7.1); −0.006(6.4); −0.008(6.2); −0.011(2.1); −0.013(1.7); −0.014(1.4); −0.015(1.3); −0.016(1.1); −0.017(1.0); −0.019(0.9); −0.020
(0.8); −0.021(0.7); −0.022(0.7); −0.024(0.7); −0.025(0.6); −0.026(0.6); −0.027(0.5); −0.028(0.5); −0.030(0.4); −0.031(0.4); −0.032(0.4); −0.033(0.4); −0.035(0.4); −0.037(0.4); −0.043(0.4); −0.048(0.5); −0.149
(0.7)
Example 280: ¹H-NMR(399.8 MHz, cdcl3):
δ = 12.519(0.5); 12.220(0.5); 7.819(3.4); 7.794(10.1); 7.774(10.2); 7.521(2.0); 7.389(0.5); 7.369(0.5); 7.351(1.8); 7.335(4.3); 7.316(4.2); 7.314(4.4); 7.298(19.9); 7.293(10.3); 7.281(25.4); 7.276(49.0);
7.262(365.1); 7.252(9.7); 7.246(12.5); 7.214(26.4); 7.193(16.0); 7.145(0.8); 7.108(0.7); 7.082(2.2); 7.078(2.3); 7.062(3.3); 7.047(6.4); 7.043(6.4); 7.027(8.6); 7.024(8.4); 7.008(4.6); 7.004(4.4); 6.998(2.7);
6.978(0.6); 6.953(0.6); 6.899(9.5); 6.896(8.5); 6.880(8.4); 6.876(7.4); 5.690(1.4); 5.675(1.6); 5.301(3.5); 5.279(3.7); 4.798(1.7); 4.781(4.7); 4.763(5.6); 4.743(3.8); 4.728(1.1); 4.554(0.8); 4.535(1.8);
4.513(2.3); 4.491(1.6); 4.472(0.6); 3.595(0.5); 3.551(0.6); 3.508(2.9); 3.489(7.0); 3.470(6.9); 3.453(2.3); 3.441(0.7); 3.403(0.5); 3.002(0.8); 2.980(2.1); 2.958(2.1); 2.935(1.0); 2.446(1.4); 2.433(1.7);
2.414(1.8); 2.400(1.5); 2.389(0.6); 2.381(0.8); 2.270(3.1); 2.256(6.8); 2.247(8.5); 2.239(6.9); 2.229(8.7); 2.219(6.4); 2.206(4.1); 2.199(4.8); 2.185(1.6); 2.177(1.0); 2.164(0.7); 2.073(0.6); 2.058(0.6);
2.022(1.2); 2.001(2.4); 1.993(2.6); 1.970(7.1); 1.962(7.8); 1.954(6.2); 1.948(5.2); 1.939(5.5); 1.910(5.1); 1.890(4.7); 1.879(3.8); 1.859(4.2); 1.843(3.2); 1.824(6.5); 1.816(6.2); 1.810(7.4); 1.790(4.5);
1.772(3.6); 1.749(2.5); 1.739(2.6); 1.717(2.6); 1.706(1.6); 1.697(1.2); 1.686(1.5); 1.664(0.9); 1.615(0.9); 1.549(147.6); 1.254(2.6); 0.900(0.6); 0.882(0.7); 0.864(0.7); 0.809(0.5); 0.147(2.1); 0.130(0.6);
0.085(1.0); 0.051(2.2); 0.000(411.3); −0.008(16.3); −0.019(1.3); −0.050(0.9); −0.149(1.8); −1.428(0.6)
Example 281: ¹H-NMR(399.8 MHz, cdcl3):
δ = 8.437(2.4); 8.432(2.5); 8.425(2.6); 8.420(2.6); 8.396(7.6); 8.391(8.0); 8.384(7.9); 8.379(7.6); 8.033(2.6); 8.028(2.6); 8.014(2.6); 8.009(2.6); 7.827(8.2); 7.822(8.1); 7.808(8.9); 7.803(8.4); 7.520(1.0);
7.467(0.4); 7.402(0.4); 7.378(0.5); 7.362(0.5); 7.325(3.6); 7.313(4.0); 7.306(4.3); 7.293(9.6); 7.287(19.4); 7.282(11.0); 7.277(17.3); 7.271(26.2); 7.261(174.7); 7.245(17.4); 7.223(5.1); 7.201(25.9);
7.180(16.0); 7.095(0.3); 7.081(0.4); 6.997(1.2); 6.435(1.2); 6.417(1.2); 6.060(3.5); 6.043(3.4); 4.786(1.7); 4.769(4.4); 4.751(5.9); 4.734(4.1); 4.716(1.3); 4.548(0.6); 4.527(1.6); 4.505(2.1); 4.484(1.6);
4.464(0.6); 3.522(0.5); 3.500(2.9); 3.481(6.8); 3.462(6.5); 3.444(2.4); 3.007(0.9); 2.983(2.0); 2.962(2.0); 2.938(1.0); 2.457(0.6); 2.437(1.2); 2.424(1.6); 2.405(1.6); 2.390(1.3); 2.371(0.8); 2.280(4.7);
2.271(6.0); 2.259(7.8); 2.244(6.6); 2.239(7.0); 2.224(5.3); 2.215(3.5); 2.208(3.2); 2.194(1.6); 2.185(0.8); 2.174(0.7); 2.134(0.4); 2.072(0.4); 2.033(1.0); 2.012(2.6); 2.004(2.9); 1.984(7.3); 1.980(7.2);
1.976(7.2); 1.964(8.6); 1.952(5.6); 1.942(3.9); 1.932(5.1); 1.922(3.1); 1.910(3.7); 1.898(2.7); 1.887(3.6); 1.878(2.3); 1.865(3.2); 1.859(3.1); 1.849(3.3); 1.838(3.5); 1.826(6.4); 1.821(6.1); 1.817(6.1);
1.808(7.5); 1.802(5.5); 1.782(4.8); 1.768(3.3); 1.746(2.0); 1.726(1.3); 1.706(1.7); 1.693(1.0); 1.685(1.7); 1.673(1.5); 1.654(1.2); 1.631(0.6); 1.603(0.6); 1.552(94.2); 1.255(0.4); 0.845(0.4); 0.146(1.1);
0.087(0.4); −0.001(200.6); −0.150(1.0)
Example 282: ¹H-NMR(399.8 MHz, cdcl3):
δ = 7.521(0.5); 7.363(0.5); 7.347(0.7); 7.342(0.7); 7.328(1.9); 7.312(3.3); 7.307(3.3); 7.296(2.5); 7.291(8.0); 7.283(12.3); 7.278(6.4); 7.276(6.5); 7.270(10.6); 7.262(111.9); 7.247(1.9); 7.242(4.8);
7.226(1.1); 7.221(2.0); 7.184(16.0); 7.163(10.5); 6.997(0.6); 6.927(0.5); 6.921(1.8); 6.901(2.7); 6.888(1.4); 6.885(1.9); 6.879(9.3); 6.858(12.5); 6.850(1.7); 6.838(7.3); 6.832(1.6); 5.874(0.4); 5.855(0.4);
5.440(1.8); 5.428(1.8); 4.793(1.1); 4.775(3.3); 4.756(4.3); 4.738(3.1); 4.720(1.0); 4.505(0.6); 4.484(0.9); 4.462(0.6); 3.495(1.6); 3.478(4.0); 3.460(4.0); 3.442(1.6); 2.986(0.4); 2.962(0.9); 2.942(0.8);
2.918(0.4); 2.431(0.5); 2.417(0.7); 2.399(0.7); 2.384(0.5); 2.269(1.3); 2.246(3.6); 2.232(2.5); 2.223(4.0); 2.215(3.1); 2.210(2.8); 2.203(4.5); 2.193(3.3); 2.188(2.3); 2.184(2.2); 2.178(1.9); 2.171(2.0);
2.161(1.8); 2.015(0.5); 2.006(0.7); 1.994(1.3); 1.984(2.1); 1.972(3.1); 1.965(4.4); 1.962(4.6); 1.955(4.8); 1.944(3.4); 1.940(3.7); 1.936(3.5); 1.922(2.4); 1.919(2.0); 1.900(2.0); 1.882(2.2); 1.872(1.2);
1.861(1.9); 1.854(2.2); 1.845(1.8); 1.831(1.9); 1.825(2.3); 1.811(2.1); 1.802(1.6); 1.788(1.7); 1.782(0.9); 1.769(0.8); 1.763(1.7); 1.744(2.4); 1.732(1.7); 1.725(2.1); 1.712(2.8); 1.693(2.5); 1.681(0.6);
1.671(1.4); 1.662(0.6); 1.640(0.6); 1.554(42.6); 1.254(0.8); 0.146(0.5); 0.008(5.3); 0.000(107.3); −0.008(4.2); −0.150(0.5)
Example 284: ¹H-NMR(399.8 MHz, cdcl3):
δ = 7.312(0.5); 7.305(3.0); 7.300(1.1); 7.289(1.4); 7.284(4.2); 7.277(0.8); 7.274(0.6); 7.262(30.2); 7.167(0.6); 7.161(3.6); 7.157(1.2); 7.144(1.1); 7.140(2.7); 5.179(0.4); 5.161(0.5); 4.642(0.8); 4.624(1.1);
4.605(0.8); 3.466(0.4); 3.448(0.9); 3.429(0.9); 3.411(0.4); 2.608(16.0); 2.332(15.8); 2.259(0.4); 2.241(0.7); 2.236(0.8); 2.229(0.6); 2.225(0.7); 2.217(1.0); 2.213(0.9); 2.204(0.5); 2.193(0.8); 2.188(0.6);
2.179(0.3); 2.170(0.4); 1.990(0.3); 1.982(0.8); 1.976(0.6); 1.968(0.9); 1.963(1.1); 1.960(1.1); 1.956(1.0); 1.943(0.9); 1.927(0.5); 1.827(0.4); 1.820(0.5); 1.808(0.5); 1.798(0.5); 1.785(0.5); 1.777(0.3);
1.702(0.5); 1.691(0.4); 1.683(0.5); 1.670(0.5); 1.651(0.4); 1.556(9.9); 0.008(1.6); 0.000(35.7); −0.008(1.4)
Example 285: ¹H-NMR(400.0 MHz, CD3CN):
δ = 7.810(10.4); 7.806(10.7); 7.790(10.7); 7.787(11.2); 7.755(7.0); 7.735(9.2); 7.690(3.2); 7.670(3.9); 7.647(10.7); 7.627(14.8); 7.604(4.0); 7.578(3.1); 7.560(2.9); 7.545(5.1); 7.525(1.9); 7.403(2.1);
7.383(3.5); 7.369(9.1); 7.367(9.6); 7.362(7.1); 7.359(6.7); 7.350(15.1); 7.348(16.0); 7.344(10.8); 7.341(8.1); 7.332(9.5); 7.329(9.6); 7.325(6.3); 7.322(5.0); 7.318(4.0); 7.313(3.7); 7.310(2.6); 7.299(2.2);
7.294(2.1); 7.105(10.3); 7.099(8.5); 7.089(11.9); 7.085(12.2); 7.078(10.1); 7.071(10.5); 7.066(7.1); 7.058(5.7); 7.054(4.4); 7.051(5.0); 7.047(3.7); 7.039(1.2); 6.800(2.8); 6.795(3.7); 6.789(2.4); 6.781(2.7);
6.775(3.4); 6.770(2.1); 6.685(3.3); 6.670(3.3); 6.540(1.3); 6.527(1.4); 4.751(1.1); 4.728(4.2); 4.706(6.4); 4.686(5.4); 4.669(2.3); 4.663(2.9); 4.646(1.3); 4.639(0.7); 4.630(0.5); 3.634(0.8); 3.613(2.1);
3.593(2.1); 3.573(0.9); 3.380(2.0); 3.357(5.6); 3.334(5.6); 3.311(2.1); 2.310(1.7); 2.304(1.7); 2.292(4.7); 2.285(3.9); 2.279(5.4); 2.273(6.2); 2.260(6.6); 2.255(6.0); 2.247(6.0); 2.241(7.0); 2.228(5.9);
2.221(5.5); 2.207(4.2); 2.200(3.9); 2.189(2.9); 2.178(1.9); 2.131(14.2); 2.124(14.1); 2.112(0.8); 2.106(1.0); 2.099(1.0); 2.093(0.7); 2.087(0.4); 2.039(1.9); 2.034(1.8); 2.019(4.3); 2.012(3.6); 1.999(4.2);
1.976(4.0); 1.971(4.1); 1.963(8.1); 1.961(7.2); 1.955(16.2); 1.951(47.4); 1.950(44.6); 1.945(98.2); 1.944(95.4); 1.938(138.0); 1.931(132.9); 1.925(84.0); 1.919(33.4); 1.908(6.2); 1.902(7.1); 1.880(6.3);
1.865(4.2); 1.859(4.9); 1.852(3.2); 1.846(3.9); 1.839(3.2); 1.833(2.7); 1.827(3.4); 1.820(2.2); 1.808(4.4); 1.802(3.2); 1.788(5.5); 1.783(5.5); 1.767(5.4); 1.761(6.2); 1.737(5.7); 1.711(5.5); 1.699(2.6);
1.686(4.8); 1.681(4.6); 1.660(3.1); 1.656(3.0); 1.636(1.1); 1.630(1.0); 1.436(7.1); 1.430(6.4); 1.269(0.4); 1.221(0.4); 1.203(0.7); 1.196(0.6); 1.185(0.4); 1.178(0.3); 0.000(56.5); −0.002(43.9); −0.008(51.6); −0.016
(1.6)
Example 286: ¹H-NMR(400.0 MHz, CD3CN):
δ = 7.737(6.8); 7.717(10.1); 7.705(3.4); 7.678(7.6); 7.665(12.1); 7.661(12.0); 7.647(16.0); 7.629(8.5); 7.606(7.4); 7.586(11.2); 7.568(7.6); 7.565(6.9); 7.555(7.2); 7.553(7.2); 7.544(4.0); 7.536(9.5);
7.532(10.8); 7.526(3.1); 7.517(4.5); 7.499(0.7); 7.420(1.7); 7.403(3.1); 7.392(5.2); 7.373(7.8); 7.354(3.6); 7.258(7.9); 7.240(7.0); 6.906(2.5); 6.901(2.8); 6.885(2.5); 6.791(2.4); 6.774(2.4); 6.610(1.0);
6.590(1.0); 4.754(1.2); 4.732(4.3); 4.711(7.0); 4.689(5.3); 4.680(1.7); 4.669(2.2); 4.652(0.6); 4.067(0.4); 4.049(0.4); 3.652(0.8); 3.631(1.7); 3.611(1.6); 3.590(0.8); 3.386(1.8); 3.363(4.6); 3.340(4.7);
3.317(1.8); 2.299(1.9); 2.285(2.4); 2.279(4.6); 2.266(5.9); 2.259(4.1); 2.248(7.1); 2.234(5.9); 2.227(5.8); 2.215(4.7); 2.206(4.5); 2.195(3.3); 2.185(1.7); 2.175(1.7); 2.132(23.2); 2.118(0.5); 2.112(0.6);
2.106(0.7); 2.100(0.6); 2.094(0.4); 2.052(2.0); 2.038(2.4); 2.031(3.6); 2.020(3.2); 2.001(1.4); 1.991(1.5); 1.971(3.5); 1.963(7.9); 1.956(14.2); 1.951(48.0); 1.945(82.1); 1.938(103.1); 1.932(71.8);
1.926(37.8); 1.914(5.2); 1.910(4.6); 1.905(4.3); 1.902(4.5); 1.895(4.0); 1.890(6.0); 1.882(2.9); 1.873(4.8); 1.867(4.2); 1.853(4.2); 1.847(2.2); 1.834(2.8); 1.821(1.6); 1.815(1.1); 1.801(1.0); 1.785(0.5);
1.779(0.6); 1.773(1.0); 1.764(4.0); 1.754(1.7); 1.745(6.1); 1.732(4.9); 1.724(7.9); 1.713(5.6); 1.702(5.6); 1.693(5.5); 1.678(3.9); 1.670(4.5); 1.655(1.8); 1.647(2.8); 1.624(1.3); 1.437(0.9); 1.221(0.5);
1.203(1.0); 1.185(0.5); 0.008(4.1); 0.000(62.9); −0.009(2.0)
Example 287: ¹H-NMR(400.0 MHz, CD3CN):
δ = 7.764(3.5); 7.712(1.3); 7.692(1.9); 7.620(3.3); 7.600(4.4); 7.581(0.8); 7.514(0.5); 7.494(0.8); 7.446(0.3); 7.428(0.6); 7.354(1.0); 7.335(1.7); 7.315(1.1); 7.293(0.6); 7.226(1.7); 7.090(3.5); 6.960(0.8);
6.955(1.8); 6.824(1.3); 6.689(0.7); 6.656(0.5); 6.636(0.5); 4.701(0.9); 4.679(1.3); 4.658(1.1); 4.636(0.5); 4.068(0.4); 4.050(0.4); 3.863(5.7); 3.850(16.0); 3.396(0.4); 3.372(1.0); 3.349(1.0); 3.327(0.4);
2.264(0.5); 2.250(0.8); 2.244(1.1); 2.232(1.3); 2.224(1.3); 2.212(1.4); 2.203(1.1); 2.194(1.0); 2.179(0.6); 2.172(0.4); 2.136(6.0); 2.032(0.5); 2.021(0.6); 2.015(0.7); 2.005(0.7); 1.990(0.4); 1.972(2.0);
1.964(1.5); 1.957(2.6); 1.952(11.9); 1.945(20.8); 1.939(27.5); 1.933(18.6); 1.927(9.6); 1.921(1.1); 1.917(0.9); 1.914(0.8); 1.910(1.5); 1.898(1.1); 1.893(1.2); 1.888(1.6); 1.878(0.8); 1.874(1.0); 1.868(0.8);
1.855(0.6); 1.765(0.8); 1.755(0.5); 1.752(0.5); 1.745(1.5); 1.734(1.3); 1.724(1.2); 1.714(1.7); 1.703(0.9); 1.692(1.3); 1.679(0.7); 1.672(0.4); 1.666(0.4); 1.657(0.6); 1.221(0.5); 1.204(0.9); 1.186(0.5);
0.008(0.6); 0.000(18.3); −0.009(0.6)
Example 288: ¹H-NMR(400.0 MHz, CD3CN):
δ = 7.672(1.5); 7.652(2.1); 7.607(1.8); 7.593(4.3); 7.590(3.7); 7.549(1.0); 7.531(1.7); 7.511(0.8); 7.313(1.1); 7.294(1.8); 7.274(0.8); 7.126(1.7); 6.990(3.5); 6.855(1.8); 6.266(0.6); 6.245(0.6); 4.414(0.7);
4.405(0.6); 4.388(0.7); 4.380(0.6); 4.068(0.4); 4.050(0.4); 3.796(16.0); 3.006(0.5); 3.002(0.5); 2.977(0.9); 2.973(0.9); 2.949(0.5); 2.945(0.4); 2.135(5.6); 2.040(0.8); 2.035(0.9); 2.023(0.5); 2.008(0.8);
1.971(1.9); 1.963(1.1); 1.957(1.9); 1.951(8.9); 1.945(15.8); 1.939(20.7); 1.933(14.2); 1.927(7.2); 1.914(0.3); 1.905(0.6); 1.900(0.8); 1.893(0.8); 1.868(1.4); 1.861(1.3); 1.850(0.8); 1.842(1.0); 1.837(1.0);
1.815(0.6); 1.807(0.9); 1.800(0.6); 1.791(0.4); 1.783(0.7); 1.774(0.9); 1.768(0.7); 1.601(0.4); 1.592(0.4); 1.580(0.6); 1.571(1.3); 1.561(0.9); 1.547(0.8); 1.539(1.7); 1.529(1.4); 1.522(0.9); 1.516(0.8);
1.505(1.6); 1.499(1.3); 1.478(0.7); 1.470(0.8); 1.421(0.5); 1.413(0.4); 1.398(0.5); 1.389(1.0); 1.381(0.6); 1.366(0.4); 1.357(0.8); 1.348(0.5); 1.221(0.5); 1.203(0.9); 1.186(0.5); 0.008(0.4); 0.000(12.3); −0.009
(0.4)
Example 289: ¹H-NMR(600.1 MHz, DMSO):
δ = 8.750(6.6); 8.748(7.1); 8.743(6.9); 8.741(6.9); 8.706(5.6); 8.692(5.7); 7.750(4.6); 7.743(8.7); 7.737(9.8); 7.729(16.0); 7.715(9.5); 7.712(10.1); 7.702(4.4); 7.699(4.2); 7.674(4.4); 7.661(7.6); 7.654(8.4);
7.641(9.2); 7.416(4.4); 7.403(7.6); 7.390(3.6); 4.670(1.0); 4.656(3.7); 4.643(5.4); 4.629(3.7); 4.616(1.1); 3.338(1.5); 3.322(30.7); 3.308(4.1); 3.294(1.6); 2.614(0.4); 2.523(0.7); 2.520(0.8); 2.517(0.8);
2.508(23.0); 2.505(49.9); 2.502(69.0); 2.499(49.6); 2.496(22.8); 2.386(0.4); 2.191(1.2); 2.178(2.8); 2.168(3.9); 2.166(3.3); 2.158(5.1); 2.152(3.7); 2.147(4.8); 2.145(4.5); 2.138(3.5); 2.134(2.7); 2.131(2.7);
2.124(1.3); 2.118(1.2); 2.037(0.9); 1.996(1.0); 1.989(0.8); 1.898(0.4); 1.891(0.5); 1.883(0.9); 1.877(1.7); 1.870(1.8); 1.865(2.5); 1.859(2.3); 1.851(2.4); 1.844(1.9); 1.838(1.9); 1.831(1.1); 1.824(2.3);
1.813(2.5); 1.810(2.9); 1.800(2.8); 1.797(1.7); 1.792(1.4); 1.788(1.8); 1.778(1.4); 1.776(0.9); 1.765(0.8); 1.751(1.8); 1.739(3.0); 1.727(2.3); 1.725(2.5); 1.718(2.9); 1.704(2.1); 1.692(1.0); 1.634(1.4);
1.620(2.9); 1.613(1.6); 1.604(3.0); 1.599(3.0); 1.589(1.4); 1.583(2.7); 1.568(1.0); 1.397(0.7); 1.175(0.4); 0.000(3.9)
Example 291: ¹H-NMR(299.9 MHz, dmso):
δ = 7.668(1.0); 7.638(1.0); 7.378(1.1); 7.372(1.3); 7.362(1.4); 7.355(2.4); 7.347(1.7); 7.337(1.8); 7.331(1.8); 7.257(0.5); 7.252(0.6); 7.233(1.5); 7.227(1.4); 7.208(1.4); 7.204(1.9); 7.197(1.6); 7.179(1.3);
7.173(1.3); 7.154(0.4); 7.148(0.4); 4.744(0.5); 4.730(0.6); 4.715(0.6); 4.701(0.5); 3.572(0.5); 3.550(0.9); 3.527(0.6); 3.511(0.8); 3.488(0.4); 3.314(48.8); 2.535(15.7); 2.512(10.8); 2.507(22.2); 2.500(30.9);
2.494(23.5); 2.489(11.7); 2.187(0.5); 2.174(0.6); 2.161(0.8); 2.148(0.9); 2.116(1.0); 2.075(16.0); 2.050(0.3); 1.919(0.6); 1.892(0.8); 1.868(1.0); 1.847(0.7); 1.829(0.5); 1.742(0.6); 1.727(0.7); 1.715(0.5);
1.699(0.7); 1.683(0.5); 1.672(0.5); 1.658(0.4); 1.645(0.4); 1.620(0.5); 1.587(0.4); 1.236(0.4); 0.011(0.9); 0.000(22.5); −0.011(1.1)
Example 292: ¹H-NMR(299.9 MHz, dmso):
δ = 7.955(0.9); 7.815(4.3); 7.425(0.4); 7.420(0.4); 7.381(1.5); 7.375(1.4); 7.356(1.8); 7.350(1.7); 7.328(1.3); 7.322(1.1); 7.303(1.9); 7.297(1.8); 7.284(0.4); 7.280(0.4); 7.205(1.7); 7.199(1.2); 7.180(2.7);
7.174(2.5); 7.155(2.5); 7.147(2.0); 7.129(1.5); 7.122(1.4); 7.104(0.5); 7.098(0.4); 4.713(0.5); 4.701(0.6); 4.691(0.5); 4.682(0.6); 3.724(0.4); 3.710(3.5); 3.698(16.0); 3.623(0.5); 3.552(0.5); 3.529(1.0);
3.507(0.8); 3.491(0.9); 3.468(0.6); 3.314(31.3); 2.512(7.0); 2.506(14.2); 2.500(19.5); 2.494(14.3); 2.488(6.9); 2.455(0.4); 2.222(3.1); 2.194(0.4); 2.185(0.7); 2.164(0.5); 2.150(0.9); 2.139(1.1); 2.122(1.0);
2.111(1.1); 2.097(0.8); 2.082(0.7); 2.071(0.7); 2.044(0.4); 1.999(15.1); 1.971(0.7); 1.950(0.4); 1.936(0.6); 1.929(0.6); 1.916(0.5); 1.900(0.6); 1.891(0.8); 1.867(1.1); 1.845(0.7); 1.829(0.6); 1.822(0.6);
1.800(0.5); 1.727(0.3); 1.714(0.3); 1.703(0.6); 1.689(0.7); 1.676(0.8); 1.660(1.0); 1.648(1.1); 1.635(0.9); 1.627(0.9); 1.597(0.6); 0.011(0.6); 0.000(14.1); −0.011(0.6)
Example 293: ¹H-NMR(299.9 MHz, dmso):
δ = 8.678(0.7); 8.649(0.7); 8.429(1.1); 8.423(1.1); 8.413(1.3); 8.407(1.3); 8.384(6.3); 8.378(6.5); 8.368(6.7); 8.362(6.1); 8.298(4.2); 8.267(4.1); 7.724(1.0); 7.718(1.0); 7.699(1.2); 7.692(1.2); 7.499(1.0);
7.469(1.4); 7.461(1.4); 7.445(1.5); 7.436(1.4); 7.406(6.3); 7.401(7.2); 7.396(9.1); 7.381(16.0); 7.371(11.5); 7.366(9.1); 7.356(11.0); 7.319(4.2); 7.314(3.8); 7.295(8.1); 7.290(6.5); 7.270(4.9); 7.260(6.6);
7.253(5.9); 7.234(6.3); 7.228(5.9); 7.210(2.4); 7.204(2.1); 7.193(0.8); 7.175(7.5); 7.168(7.3); 7.149(6.1); 7.143(5.6); 4.827(1.2); 4.817(1.4); 4.803(2.6); 4.795(3.2); 4.783(2.5); 4.772(3.3); 4.751(1.4);
4.739(1.1); 4.600(0.6); 4.572(0.8); 4.543(0.6); 3.585(2.0); 3.563(4.0); 3.543(3.5); 3.523(3.8); 3.501(1.9); 3.462(0.8); 3.434(0.8); 3.320(26.0); 2.507(29.9); 2.501(38.0); 2.495(27.5); 2.418(0.3); 2.257(1.6);
2.227(2.3); 2.212(3.0); 2.201(3.1); 2.186(3.6); 2.175(3.0); 2.157(2.4); 2.134(2.8); 2.102(3.6); 2.074(2.3); 2.063(2.1); 2.036(1.2); 1.960(0.6); 1.908(3.5); 1.897(3.4); 1.880(5.8); 1.857(3.8); 1.841(3.6);
1.811(2.2); 1.781(1.1); 1.750(1.6); 1.739(1.6); 1.726(2.9); 1.715(2.7); 1.700(4.0); 1.673(5.4); 1.657(4.3); 1.626(2.2); 1.597(1.1); 1.547(0.6); 1.515(0.6); 1.506(0.6); 1.473(0.5); 1.397(1.1); 1.237(1.0);
0.958(0.4); 0.936(0.4); 0.880(0.5); 0.859(1.6); 0.835(1.3); 0.814(0.4); 0.000(19.6)
Example 294: ¹H-NMR(299.9 MHz, dmso):
δ = 8.499(1.5); 8.471(1.5); 8.128(4.0); 8.098(4.0); 7.494(2.0); 7.469(2.6); 7.434(1.2); 7.408(10.7); 7.404(11.1); 7.397(6.8); 7.383(14.3); 7.378(15.1); 7.373(9.3); 7.362(11.7); 7.357(16.0); 7.335(8.2);
7.331(8.1); 7.317(5.3); 7.311(6.2); 7.305(4.0); 7.292(7.9); 7.288(6.4); 7.267(8.3); 7.259(9.2); 7.250(8.2); 7.243(10.2); 7.237(8.5); 7.234(8.4); 7.226(7.4); 7.219(6.7); 7.214(5.3); 7.202(2.0); 7.194(1.0);
7.189(0.9); 6.732(6.4); 6.709(5.3); 4.832(1.0); 4.821(1.2); 4.809(2.2); 4.799(2.7); 4.788(2.1); 4.777(2.7); 4.755(1.3); 4.742(1.0); 4.637(0.4); 4.609(1.1); 4.580(1.6); 4.552(1.1); 4.524(0.3); 3.582(1.9);
3.560(3.7); 3.539(3.0); 3.520(3.5); 3.499(2.0); 3.472(1.5); 3.443(1.6); 3.412(0.7); 3.315(66.9); 2.727(0.6); 2.507(49.6); 2.501(65.0); 2.496(48.7); 2.276(0.8); 2.245(1.3); 2.233(1.5); 2.214(2.5); 2.199(2.9);
2.189(3.5); 2.169(3.8); 2.154(3.4); 2.145(2.9); 2.120(3.5); 2.093(2.2); 2.054(1.0); 1.964(0.6); 1.908(2.2); 1.883(3.1); 1.865(4.7); 1.844(3.8); 1.825(3.4); 1.800(3.2); 1.773(1.9); 1.751(2.2); 1.738(1.5);
1.725(2.7); 1.713(2.5); 1.699(3.0); 1.684(3.4); 1.670(3.3); 1.659(4.8); 1.631(2.7); 1.599(1.9); 1.568(0.8); 1.531(1.0); 1.497(1.0); 1.489(1.0); 1.456(0.7); 1.428(0.3); 1.235(0.8); 0.002(36.2); 0.000(34.0); −0.008
(1.7)
Example 295: ¹H-NMR(299.9 MHz, dmso):
δ = 8.459(1.6); 8.431(1.6); 8.074(5.7); 8.043(5.7); 7.847(0.3); 7.828(2.8); 7.804(3.1); 7.781(10.1); 7.779(9.6); 7.755(11.2); 7.752(9.8); 7.508(2.1); 7.504(2.1); 7.482(2.9); 7.478(2.7); 7.460(0.5); 7.410(10.5);
7.403(9.7); 7.397(10.2); 7.385(16.0); 7.378(13.6); 7.372(11.7); 7.363(3.4); 7.359(3.3); 7.329(6.9); 7.306(12.8); 7.301(9.6); 7.287(14.0); 7.283(14.3); 7.275(5.9); 7.262(15.6); 7.258(11.4); 7.237(10.2);
7.231(7.8); 7.211(5.3); 7.206(3.1); 7.192(1.4); 7.139(6.5); 7.115(7.0); 7.090(7.6); 7.085(7.3); 7.065(9.4); 7.059(9.1); 7.039(5.1); 7.034(4.7); 6.549(8.8); 6.544(8.7); 6.524(8.6); 6.519(7.9); 4.804(1.5);
4.793(1.9); 4.781(3.2); 4.771(3.9); 4.761(3.2); 4.749(4.0); 4.740(3.0); 4.728(1.9); 4.715(1.5); 4.609(0.3); 4.582(1.3); 4.552(1.7); 4.525(1.1); 4.497(0.4); 3.581(2.7); 3.559(5.2); 3.538(4.4); 3.519(5.1);
3.497(2.7); 3.483(1.8); 3.451(1.7); 3.420(0.8); 3.314(93.5); 2.727(0.8); 2.507(87.3); 2.501(110.6); 2.495(77.8); 2.489(35.7); 2.270(1.5); 2.252(1.4); 2.239(1.8); 2.225(2.4); 2.207(3.6); 2.196(4.0); 2.182(5.0);
2.163(6.3); 2.139(5.3); 2.129(6.1); 2.102(3.9); 2.063(1.6); 1.956(1.3); 1.917(3.1); 1.902(3.2); 1.882(4.5); 1.860(5.8); 1.839(4.4); 1.822(4.6); 1.798(5.6); 1.775(4.5); 1.762(4.4); 1.747(3.8); 1.732(4.3);
1.719(3.6); 1.704(3.3); 1.695(4.4); 1.666(3.3); 1.642(3.6); 1.629(2.7); 1.604(2.8); 1.580(1.3); 1.546(0.9); 1.520(1.0); 1.487(1.1); 1.480(1.1); 1.446(0.9); 1.418(0.4); 1.236(1.0); 1.133(0.4); 1.110(0.7);
0.011(4.6); 0.000(75.7); −0.011(3.0)
Example 296: ¹H-NMR(299.9 MHz, dmso):
δ = 8.580(0.9); 8.550(1.0); 8.186(5.1); 8.156(5.1); 7.732(1.0); 7.706(1.8); 7.683(4.8); 7.675(4.2); 7.672(4.3); 7.665(6.2); 7.653(7.1); 7.617(1.2); 7.585(2.3); 7.566(9.4); 7.561(11.4); 7.549(16.0); 7.537(10.2);
7.513(1.4); 7.482(1.3); 7.476(1.3); 7.445(5.7); 7.439(6.0); 7.420(14.6); 7.417(15.2); 7.395(10.7); 7.391(10.9); 7.358(4.0); 7.353(4.3); 7.333(8.6); 7.328(7.4); 7.308(6.0); 7.303(4.4); 7.285(7.6); 7.279(6.7);
7.260(7.4); 7.254(7.5); 7.235(3.0); 7.229(3.5); 7.197(0.6); 6.686(5.5); 6.675(5.5); 6.657(5.3); 4.835(1.2); 4.823(1.6); 4.802(3.4); 4.779(3.4); 4.756(1.6); 4.745(1.2); 4.594(0.7); 4.566(0.9); 4.537(0.7);
3.585(2.3); 3.563(4.5); 3.542(3.8); 3.523(4.4); 3.501(2.5); 3.469(1.0); 3.442(0.9); 3.410(0.4); 3.390(0.3); 3.314(96.5); 2.727(0.7); 2.512(36.4); 2.507(66.9); 2.501(85.7); 2.495(61.3); 2.271(0.8); 2.265(0.9);
2.234(1.8); 2.204(2.7); 2.178(5.1); 2.152(4.2); 2.139(5.1); 2.111(3.4); 2.072(1.2); 1.948(0.8); 1.923(2.0); 1.895(4.2); 1.874(6.2); 1.852(4.1); 1.846(4.1); 1.836(3.7); 1.813(2.3); 1.795(1.5); 1.768(1.1);
1.746(0.7); 1.706(2.3); 1.695(2.0); 1.682(4.0); 1.671(3.8); 1.657(5.3); 1.649(5.7); 1.630(6.0); 1.606(2.9); 1.577(1.0); 1.553(0.5); 1.526(0.6); 1.484(0.6); 1.451(0.5); 1.237(0.6); 0.011(3.3); 0.000(50.1); −0.011
(2.0)
Example 297: ¹H-NMR(299.9 MHz, dmso):
δ = 12.212(0.4); 8.756(1.5); 8.742(2.2); 8.714(8.4); 8.698(7.7); 8.342(6.5); 8.311(6.7); 7.794(0.7); 7.774(1.8); 7.751(1.8); 7.736(1.6); 7.726(0.7); 7.710(0.7); 7.677(7.0); 7.661(7.1); 7.651(7.8); 7.635(7.1);
7.481(1.3); 7.455(1.9); 7.429(7.9); 7.425(8.3); 7.404(16.0); 7.399(13.1); 7.386(11.3); 7.380(11.3); 7.353(5.7); 7.349(5.7); 7.329(11.7); 7.325(9.3); 7.304(6.6); 7.299(5.2); 7.288(8.1); 7.281(7.4); 7.263(8.5);
7.257(8.0); 7.238(3.7); 7.232(3.5); 7.209(0.8); 7.123(8.6); 7.097(7.8); 4.834(1.4); 4.824(1.9); 4.802(4.4); 4.779(4.4); 4.757(1.9); 4.587(0.7); 4.559(1.1); 4.531(0.7); 3.591(2.8); 3.569(5.6); 3.549(4.6);
3.530(5.4); 3.508(3.1); 3.488(0.8); 3.453(1.4); 3.425(1.4); 3.363(3.1); 3.345(8.0); 3.315(1136.0); 3.283(3.0); 3.281(3.0); 3.274(2.3); 3.269(1.8); 3.266(1.7); 3.249(1.2); 3.219(0.6); 3.197(0.4); 3.181(0.9);
3.159(0.4); 3.048(0.6); 2.913(0.4); 2.733(1.3); 2.727(1.6); 2.645(0.4); 2.512(91.5); 2.507(168.0); 2.501(214.8); 2.495(153.3); 2.490(72.3); 2.270(2.0); 2.264(1.8); 2.250(1.7); 2.220(3.3); 2.209(3.8);
2.196(4.7); 2.181(5.1); 2.170(3.8); 2.150(4.1); 2.108(4.5); 2.078(2.9); 2.069(2.7); 2.039(1.4); 1.947(1.3); 1.910(4.8); 1.890(8.1); 1.868(6.0); 1.853(5.0); 1.829(3.1); 1.803(1.8); 1.776(1.4); 1.757(0.8);
1.729(1.2); 1.705(3.6); 1.676(6.5); 1.651(8.4); 1.633(5.3); 1.610(2.2); 1.573(0.7); 1.546(0.7); 1.512(0.7); 1.504(0.7); 1.472(0.6); 1.284(0.4); 1.236(2.4); 0.853(0.4); 0.010(2.8); 0.000(45.2); −0.011(1.9)
Example 298: ¹H-NMR(400.0 MHz, DMSO):
δ = 8.681(0.5); 8.660(0.4); 8.608(3.3); 8.564(3.8); 8.286(0.4); 8.266(0.4); 8.202(4.9); 8.181(5.0); 8.120(0.4); 8.100(0.4); 7.800(3.5); 7.795(5.6); 7.791(3.7); 7.780(4.0); 7.776(6.3); 7.771(4.0); 7.677(0.3);
7.644(0.7); 7.623(1.1); 7.564(11.5); 7.544(16.0); 7.498(0.3); 7.486(0.4); 7.439(14.7); 7.418(11.1); 7.408(5.1); 7.396(4.7); 7.388(4.5); 7.376(4.1); 5.758(0.6); 4.716(1.2); 4.697(3.1); 4.679(4.1); 4.660(3.2);
4.642(1.3); 3.526(0.7); 3.476(2.3); 3.456(4.7); 3.434(4.4); 3.415(2.1); 2.673(0.4); 2.526(1.1); 2.513(26.4); 2.508(54.4); 2.504(72.6); 2.499(52.7); 2.495(25.6); 2.335(0.4); 2.331(0.5); 2.326(0.4); 2.154(1.3);
2.144(1.6); 2.135(1.8); 2.131(1.9); 2.122(3.1); 2.113(4.0); 2.104(4.0); 2.099(2.7); 2.093(3.4); 2.081(6.5); 2.072(4.3); 2.061(7.9); 2.052(5.3); 2.040(4.8); 2.032(3.4); 2.021(0.9); 2.009(0.9); 2.005(1.1);
1.998(1.6); 1.986(1.6); 1.977(2.9); 1.968(2.8); 1.958(2.8); 1.948(2.9); 1.938(1.9); 1.927(1.5); 1.918(0.9); 1.911(0.5); 1.895(0.3); 1.858(0.4); 1.840(0.4); 1.816(1.7); 1.800(1.8); 1.795(3.3); 1.779(3.3);
1.774(2.3); 1.768(1.7); 1.762(2.7); 1.759(2.4); 1.747(2.3); 1.742(2.0); 1.727(1.9); 1.721(1.3); 1.700(2.8); 1.692(1.3); 1.679(3.4); 1.671(2.7); 1.657(2.2); 1.649(2.9); 1.636(0.8); 1.627(1.7); 1.605(0.5);
0.146(0.5); 0.008(4.1); 0.000(109.0); −0.009(4.0); −0.150(0.5)
Example 299: ¹H-NMR(400.0 MHz, DMSO):
δ = 13.422(0.4); 9.078(0.8); 8.798(0.6); 8.791(0.6); 8.652(0.5); 8.587(1.0); 8.539(3.6); 8.442(0.4); 8.353(0.7); 8.317(0.8); 8.284(0.7); 8.279(1.1); 8.275(0.7); 8.264(0.8); 8.260(1.2); 8.255(0.7); 8.058(1.5);
7.913(2.9); 7.866(0.3); 7.705(5.4); 7.686(6.7); 7.596(5.8); 7.577(6.4); 7.554(2.1); 7.546(1.7); 7.534(1.5); 7.500(3.2); 7.484(3.1); 7.421(1.1); 7.404(1.0); 7.335(3.6); 7.243(6.5); 7.224(5.9); 7.043(2.2);
7.024(1.9); 5.757(6.9); 5.414(0.5); 5.162(1.0); 5.144(1.0); 5.045(0.5); 3.905(0.7); 3.883(1.7); 3.859(1.8); 3.836(0.8); 3.643(0.9); 3.623(1.0); 3.326(89.5); 3.294(1.0); 3.269(1.8); 3.248(1.6); 3.179(0.3);
3.123(0.3); 3.110(0.4); 2.977(16.0); 2.922(0.5); 2.878(0.3); 2.842(0.7); 2.815(3.3); 2.799(1.3); 2.681(0.5); 2.676(1.1); 2.672(1.6); 2.667(1.1); 2.663(0.5); 2.542(0.8); 2.525(3.6); 2.520(5.6); 2.512(87.4);
2.507(178.4); 2.503(234.7); 2.498(166.9); 2.494(78.2); 2.392(2.2); 2.334(2.3); 2.329(2.8); 2.325(2.7); 2.303(7.3); 2.229(0.4); 2.112(3.0); 2.091(3.1); 2.068(3.2); 1.989(4.1); 1.958(5.9); 1.952(5.8);
1.876(1.4); 1.842(2.0); 1.737(3.8); 1.724(3.6); 1.577(0.4); 1.398(2.6); 1.236(0.5); 1.193(0.4); 1.175(0.7); 1.157(0.3); 0.146(1.6); 0.008(12.4); 0.000(356.3); −0.009(12.3); −0.150(1.6)
Example 300: ¹H-NMR(399.8 MHz, dmso):
δ = 8.469(0.3); 8.445(0.3); 8.306(4.3); 8.283(4.4); 7.744(0.4); 7.725(0.6); 7.668(6.7); 7.649(7.8); 7.611(16.0); 7.602(13.7); 7.581(0.9); 7.434(2.5); 7.423(4.4); 7.414(4.3); 7.408(4.7); 7.404(6.6); 7.388(10.3);
7.385(10.9); 7.381(7.1); 7.377(6.4); 7.363(7.5); 7.359(7.2); 7.343(3.5); 7.339(3.2); 7.330(0.8); 7.325(0.7); 7.308(5.2); 7.303(4.8); 7.289(6.8); 7.285(6.6); 7.271(3.7); 7.267(3.3); 7.181(0.5); 7.178(0.5);
7.164(0.4); 7.159(0.4); 6.839(6.5); 6.836(6.5); 6.820(6.6); 6.817(6.1); 4.639(1.1); 4.629(1.4); 4.621(2.3); 4.612(2.5); 4.604(2.3); 4.598(2.5); 4.589(2.1); 4.580(1.3); 4.570(1.0); 3.471(1.4); 3.454(2.8);
3.440(2.5); 3.425(2.7); 3.409(1.3); 3.363(0.4); 3.314(131.9); 2.674(0.4); 2.669(0.5); 2.567(0.4); 2.536(1.0); 2.509(26.1); 2.504(52.2); 2.500(69.1); 2.495(49.6); 2.491(22.9); 2.331(0.4); 2.326(0.5);
2.237(0.9); 2.225(1.1); 2.218(1.3); 2.213(1.5); 2.203(2.2); 2.193(2.2); 2.180(2.3); 2.173(1.7); 2.168(1.7); 2.161(1.5); 2.149(1.4); 2.115(0.9); 2.086(1.6); 2.060(2.7); 2.039(2.1); 2.010(1.2); 1.960(0.9);
1.945(1.6); 1.940(2.0); 1.928(3.5); 1.910(5.6); 1.896(3.7); 1.883(3.0); 1.865(1.2); 1.839(0.4); 1.819(0.5); 1.797(1.6); 1.787(1.7); 1.778(2.3); 1.768(2.6); 1.758(2.0); 1.753(1.9); 1.745(2.5); 1.735(2.1);
1.725(1.5); 1.715(1.4); 1.682(0.9); 1.664(1.6); 1.659(1.5); 1.653(1.2); 1.640(2.4); 1.635(2.1); 1.628(1.7); 1.622(1.6); 1.615(1.9); 1.610(2.1); 1.597(0.9); 1.585(1.0); 1.565(0.4); 1.236(0.3); 0.007(2.3); −0.001
(46.0); −0.009(1.4)
Example 301: ¹H-NMR(399.8 MHz, dmso):
δ = 8.644(0.4); 8.622(0.4); 8.465(5.0); 8.442(5.1); 8.419(1.2); 8.410(8.6); 8.405(9.1); 8.397(9.0); 8.393(8.5); 7.749(0.5); 7.730(0.9); 7.672(7.0); 7.652(8.5); 7.633(2.6); 7.614(6.7); 7.594(10.0); 7.588(10.0);
7.572(2.9); 7.457(1.0); 7.444(1.4); 7.433(12.0); 7.421(11.5); 7.414(16.0); 7.402(12.2); 7.374(0.4); 7.290(10.3); 7.285(10.6); 7.271(8.4); 7.267(7.9); 4.637(0.5); 4.624(1.2); 4.615(1.7); 4.606(2.4); 4.598(3.0);
4.591(2.5); 4.583(2.9); 4.575(2.3); 4.566(1.4); 4.557(1.2); 3.479(1.5); 3.463(3.1); 3.448(2.7); 3.433(2.9); 3.418(1.5); 3.363(0.5); 3.314(190.4); 3.264(0.5); 2.669(0.8); 2.665(0.6); 2.536(1.2); 2.509(37.4);
2.505(75.9); 2.500(102.2); 2.496(72.3); 2.491(33.3); 2.450(0.4); 2.331(0.5); 2.327(0.7); 2.323(0.6); 2.251(1.0); 2.238(1.3); 2.232(1.4); 2.226(1.6); 2.217(2.6); 2.206(2.5); 2.193(2.4); 2.186(2.0); 2.182(2.0);
2.174(1.7); 2.163(1.6); 2.134(0.5); 2.114(0.4); 2.102(0.7); 2.072(1.7); 2.047(3.0); 2.026(2.2); 2.019(2.2); 1.998(1.4); 1.968(1.1); 1.965(1.1); 1.943(3.4); 1.925(5.6); 1.915(4.5); 1.910(4.4); 1.899(3.9);
1.880(1.9); 1.856(0.4); 1.843(0.6); 1.822(0.5); 1.798(1.7); 1.789(1.8); 1.780(2.3); 1.771(2.5); 1.764(2.2); 1.760(2.3); 1.755(2.1); 1.751(2.4); 1.747(2.5); 1.737(2.3); 1.727(1.7); 1.718(1.8); 1.708(1.2);
1.690(1.8); 1.680(1.3); 1.667(2.6); 1.662(2.3); 1.655(1.9); 1.649(1.8); 1.643(1.9); 1.637(2.2); 1.625(1.1); 1.618(1.1); 1.613(1.0); 1.594(0.6); 1.237(0.5); 0.008(2.8); −0.001(62.0); −0.009(2.0)
Example 302: ¹H-NMR(400.0 MHz, DMSO):
δ = 13.562(0.5); 8.317(0.5); 8.240(4.7); 8.218(4.7); 7.923(0.4); 7.848(1.6); 7.830(2.2); 7.827(2.1); 7.817(1.2); 7.799(2.3); 7.790(0.8); 7.784(1.1); 7.780(1.1); 7.765(2.2); 7.742(2.4); 7.739(2.1); 7.721(1.9);
7.703(0.8); 7.695(0.7); 7.682(5.1); 7.678(6.1); 7.665(16.0); 7.645(14.4); 7.624(0.4); 7.617(0.5); 7.604(0.4); 7.561(2.1); 7.542(5.9); 7.527(8.6); 7.523(9.0); 7.509(5.8); 7.487(14.8); 7.466(10.9); 7.441(0.6);
6.607(5.5); 6.591(5.5); 5.345(0.3); 4.720(1.0); 4.708(1.4); 4.702(2.4); 4.690(2.7); 4.685(2.7); 4.680(2.7); 4.668(2.4); 4.662(1.6); 4.650(1.1); 3.450(1.6); 3.428(3.9); 3.409(3.7); 3.387(1.7); 3.326(52.2);
3.105(0.4); 3.086(0.4); 2.675(0.7); 2.671(1.0); 2.666(0.7); 2.541(0.5); 2.524(2.4); 2.511(55.2); 2.506(111.1); 2.502(145.6); 2.497(104.1); 2.493(49.2); 2.338(0.4); 2.333(0.7); 2.329(1.0); 2.324(0.7);
2.139(1.3); 2.119(2.1); 2.108(2.6); 2.098(2.6); 2.089(3.2); 2.080(2.4); 2.069(3.0); 2.065(3.0); 2.052(5.0); 2.048(5.6); 2.044(5.7); 2.031(7.1); 2.024(4.6); 2.010(4.2); 1.949(0.4); 1.937(0.7); 1.922(1.2);
1.910(2.2); 1.899(2.8); 1.888(1.5); 1.883(2.1); 1.871(1.9); 1.852(0.8); 1.806(0.4); 1.787(0.3); 1.723(1.3); 1.711(1.8); 1.704(2.5); 1.692(4.1); 1.685(2.5); 1.673(6.3); 1.661(4.5); 1.654(4.1); 1.642(1.7);
1.634(2.7); 1.622(0.7); 1.614(1.2); 1.592(0.5); 1.192(0.4); 1.174(0.8); 1.156(0.4); 1.138(1.4); 1.120(2.9); 1.103(1.4); 1.000(1.4); 0.982(3.0); 0.964(1.4); 0.146(0.6); 0.008(5.7); 0.000(147.8); −0.009(5.1); −0.150
(0.7)
Example 303: ¹H-NMR(400.0 MHz, DMSO):
δ = 8.317(0.5); 8.179(5.4); 8.157(5.5); 7.683(0.5); 7.666(1.1); 7.647(13.4); 7.626(16.0); 7.603(0.5); 7.585(0.6); 7.555(1.9); 7.545(6.7); 7.537(6.8); 7.532(4.8); 7.530(4.8); 7.522(7.8); 7.513(1.2); 7.480(15.1);
7.460(12.1); 7.389(0.4); 7.387(0.4); 7.371(0.3); 7.368(0.3); 7.335(0.4); 7.330(0.4); 7.316(0.4); 7.311(0.5); 7.279(0.4); 7.271(2.2); 7.261(15.1); 7.252(11.8); 7.247(11.0); 7.238(15.0); 7.228(2.1); 7.214(0.4);
7.210(0.4); 6.650(0.8); 6.641(6.9); 6.633(4.9); 6.627(6.7); 6.618(6.4); 6.608(0.8); 5.340(0.4); 4.708(1.2); 4.691(2.9); 4.678(3.2); 4.674(3.3); 4.669(3.1); 4.656(2.8); 4.652(1.9); 4.638(1.3); 3.859(0.5);
3.446(1.8); 3.425(4.5); 3.405(4.4); 3.384(1.9); 3.324(130.8); 2.675(0.9); 2.671(1.3); 2.666(0.9); 2.541(0.6); 2.524(3.9); 2.510(75.6); 2.506(149.1); 2.502(194.7); 2.497(141.2); 2.333(1.0); 2.328(1.3);
2.324(1.0); 2.148(1.4); 2.135(1.4); 2.130(2.0); 2.125(2.0); 2.115(3.3); 2.106(2.9); 2.096(2.9); 2.092(2.7); 2.087(2.4); 2.075(1.8); 2.068(1.4); 2.060(2.8); 2.056(3.0); 2.043(5.3); 2.036(7.6); 2.022(8.3);
2.015(5.6); 2.000(4.6); 1.959(0.5); 1.930(1.7); 1.919(2.5); 1.907(2.8); 1.891(2.9); 1.873(1.8); 1.860(0.9); 1.826(0.4); 1.808(0.4); 1.790(0.4); 1.762(1.7); 1.749(1.7); 1.742(2.8); 1.729(3.8); 1.722(2.2);
1.717(1.8); 1.710(4.0); 1.697(3.7); 1.681(4.5); 1.661(3.5); 1.654(2.6); 1.641(2.1); 1.632(2.6); 1.621(0.8); 1.612(1.4); 1.590(0.5); 1.159(0.4); 0.991(0.4); 0.146(0.8); 0.008(7.7); 0.000(177.5); −0.008(6.8); −0.150
(0.8)
Example 304: ¹H-NMR(400.0 MHz, DMSO):
δ = 8.463(4.9); 8.441(5.1); 8.317(0.4); 7.659(0.6); 7.638(0.9); 7.594(12.3); 7.573(16.0); 7.530(0.9); 7.510(0.7); 7.472(0.4); 7.446(15.5); 7.427(13.9); 7.411(2.7); 7.407(7.2); 7.402(2.7); 7.390(3.6);
7.386(4.3); 7.369(2.0); 7.214(0.6); 7.192(0.7); 7.168(0.3); 7.137(0.5); 7.118(0.6); 7.097(0.4); 7.067(1.3); 7.064(1.8); 7.056(11.5); 7.037(14.3); 7.036(13.5); 7.016(9.9); 7.008(1.7); 5.343(0.4); 4.712(1.2);
4.700(1.7); 4.695(2.5); 4.691(2.1); 4.684(2.9); 4.678(3.0); 4.673(3.0); 4.667(2.1); 4.662(2.6); 4.657(1.5); 4.645(1.1); 3.487(0.3); 3.469(0.3); 3.418(1.7); 3.400(3.5); 3.376(3.7); 3.358(2.0); 3.326(73.4);
3.092(0.3); 3.024(0.5); 2.857(0.4); 2.679(0.3); 2.675(0.7); 2.671(0.9); 2.666(0.7); 2.541(0.5); 2.524(2.3); 2.519(3.6); 2.511(53.0); 2.506(108.3); 2.502(141.9); 2.497(101.2); 2.493(47.4); 2.338(0.4);
2.333(0.8); 2.328(1.0); 2.324(0.7); 2.151(0.7); 2.132(1.4); 2.122(2.0); 2.113(2.9); 2.104(2.5); 2.102(2.5); 2.096(2.3); 2.093(2.4); 2.084(3.1); 2.067(2.2); 2.058(2.9); 2.047(3.6); 2.037(4.3); 2.026(5.7);
2.008(3.0); 2.000(3.7); 1.993(1.7); 1.978(2.1); 1.973(1.3); 1.968(1.2); 1.947(1.5); 1.936(0.9); 1.931(0.8); 1.924(1.5); 1.915(2.4); 1.904(2.7); 1.894(3.4); 1.884(2.4); 1.873(1.9); 1.866(1.2); 1.855(0.9);
1.843(0.4); 1.837(0.3); 1.811(0.4); 1.801(0.3); 1.791(0.4); 1.740(0.7); 1.720(1.8); 1.710(1.0); 1.702(3.8); 1.690(2.1); 1.682(4.8); 1.673(5.6); 1.662(5.5); 1.653(3.1); 1.644(2.9); 1.631(2.0); 1.626(1.3);
1.614(1.0); 1.193(0.4); 1.175(0.8); 1.162(0.4); 1.156(0.4); 1.145(0.9); 1.127(0.4); 1.005(0.6); 0.146(0.6); 0.016(0.3); 0.008(5.1); 0.000(152.6); −0.009(5.1); −0.150(0.6)
Example 305: ¹H-NMR(400.0 MHz, DMSO):
δ = 8.317(0.3); 8.189(5.2); 8.167(5.3); 7.679(0.6); 7.664(0.7); 7.654(0.7); 7.637(12.8); 7.616(16.0); 7.549(0.9); 7.528(0.7); 7.504(0.4); 7.474(15.0); 7.454(12.0); 7.429(0.8); 7.426(0.8); 7.418(0.7);
7.414(0.6); 7.407(0.4); 7.401(0.7); 7.396(0.6); 7.383(3.6); 7.380(4.7); 7.363(11.9); 7.360(11.7); 7.351(6.2); 7.347(6.5); 7.333(7.3); 7.329(7.4); 7.313(3.1); 7.309(3.1); 7.267(0.5); 7.263(0.5); 7.249(0.4);
7.244(0.4); 7.231(4.7); 7.227(4.6); 7.212(7.7); 7.209(7.6); 7.194(4.0); 7.190(3.8); 6.717(7.4); 6.713(7.6); 6.698(7.2); 6.694(7.0); 5.339(0.5); 4.714(1.1); 4.697(2.8); 4.684(3.2); 4.679(3.3); 4.675(3.1);
4.662(2.8); 4.657(1.9); 4.644(1.3); 3.448(1.8); 3.427(4.5); 3.408(4.4); 3.386(1.9); 3.326(86.1); 2.675(0.6); 2.670(0.8); 2.666(0.6); 2.541(0.4); 2.523(2.0); 2.510(48.2); 2.506(94.7); 2.501(122.7); 2.497(87.8);
2.493(42.0); 2.333(0.7); 2.328(0.9); 2.324(0.6); 2.152(1.4); 2.133(2.0); 2.129(2.2); 2.119(3.0); 2.110(3.1); 2.100(3.2); 2.091(2.6); 2.078(2.0); 2.070(0.7); 2.056(3.5); 2.041(5.0); 2.034(8.7); 2.019(7.8);
2.012(5.9); 1.998(4.0); 1.965(0.4); 1.956(0.7); 1.946(0.5); 1.937(1.0); 1.925(1.9); 1.918(2.5); 1.904(2.7); 1.889(2.9); 1.873(2.2); 1.859(0.9); 1.829(0.4); 1.810(0.5); 1.791(0.5); 1.783(0.3); 1.749(1.6);
1.736(1.7); 1.730(2.9); 1.717(3.9); 1.710(2.4); 1.704(1.7); 1.697(4.1); 1.685(3.2); 1.678(4.2); 1.673(4.3); 1.658(3.7); 1.651(2.7); 1.638(2.2); 1.629(2.8); 1.617(0.8); 1.608(1.6); 1.587(0.5); 1.153(0.6);
1.136(0.3); 0.986(0.6); 0.146(0.5); 0.008(4.7); 0.000(112.6); −0.009(4.0); −0.150(0.5)
Example 306: ¹H-NMR(400.0 MHz, DMSO):
δ = 8.373(5.1); 8.351(5.2); 8.317(0.8); 8.013(0.4); 8.010(0.4); 7.993(0.5); 7.990(0.5); 7.981(0.5); 7.976(0.9); 7.967(6.9); 7.961(4.6); 7.955(5.6); 7.952(4.6); 7.944(7.8); 7.935(1.1); 7.755(0.5); 7.752(0.5);
7.736(0.3); 7.733(0.4); 7.678(1.4); 7.658(12.4); 7.638(15.1); 7.622(2.1); 7.618(1.5); 7.609(16.0); 7.603(8.5); 7.599(10.4); 7.597(10.3); 7.592(8.1); 7.586(13.2); 7.578(1.2); 7.574(1.9); 7.567(0.5); 7.550(0.7);
7.530(0.6); 7.473(13.7); 7.452(11.6); 7.439(0.6); 7.435(0.5); 6.775(0.8); 6.765(7.3); 6.757(4.6); 6.755(5.5); 6.753(4.9); 6.748(4.6); 6.742(7.0); 6.734(0.7); 6.728(0.3); 5.757(1.3); 5.366(0.4); 4.681(1.0);
4.663(2.5); 4.651(2.8); 4.646(2.9); 4.642(2.8); 4.629(2.5); 4.624(1.7); 4.611(1.1); 3.453(1.7); 3.434(3.4); 3.411(3.6); 3.392(1.7); 3.324(149.7); 2.680(0.5); 2.675(1.2); 2.671(1.6); 2.666(1.2); 2.662(0.5);
2.541(0.9); 2.524(4.5); 2.519(7.3); 2.511(88.4); 2.506(180.2); 2.502(238.5); 2.497(170.2); 2.493(80.0); 2.338(0.6); 2.333(1.2); 2.328(1.6); 2.324(1.2); 2.319(0.6); 2.136(1.3); 2.114(2.2); 2.103(2.7);
2.094(3.0); 2.081(4.2); 2.069(3.6); 2.063(4.8); 2.050(4.2); 2.043(5.2); 2.032(2.8); 2.019(3.6); 1.997(1.8); 1.991(1.1); 1.987(1.2); 1.967(1.0); 1.952(0.6); 1.946(0.5); 1.933(1.7); 1.923(2.3); 1.913(3.1);
1.903(2.4); 1.894(2.4); 1.884(1.7); 1.874(1.3); 1.863(0.9); 1.831(0.3); 1.812(0.4); 1.794(0.4); 1.757(1.6); 1.744(1.7); 1.738(3.1); 1.725(3.5); 1.719(3.6); 1.706(5.0); 1.700(4.3); 1.688(4.1); 1.683(4.2);
1.668(2.0); 1.660(2.3); 1.650(0.7); 1.641(1.3); 1.619(0.4); 0.146(1.0); 0.008(9.5); 0.000(257.3); −0.009(8.5); −0.020(0.4); −0.150(1.1)
Example 307: ¹H-NMR(400.0 MHz, DMSO):
δ = 8.317(0.3); 7.802(0.6); 7.783(0.9); 7.769(1.6); 7.754(4.0); 7.735(5.3); 7.719(2.2); 7.699(0.7); 7.682(1.0); 7.666(0.9); 7.661(0.9); 7.638(1.3); 7.617(1.1); 7.589(0.3); 7.561(1.4); 7.541(5.9); 7.521(5.7);
7.504(3.3); 7.486(2.2); 7.411(0.3); 7.008(0.3); 6.989(0.3); 6.814(0.7); 6.796(0.7); 6.262(1.7); 6.244(1.7); 5.757(2.1); 5.271(0.5); 5.251(1.5); 5.230(1.6); 5.210(0.6); 3.975(0.6); 3.957(0.6); 3.663(0.4);
3.642(1.1); 3.620(1.1); 3.598(0.5); 3.324(100.6); 3.181(0.4); 3.159(0.5); 2.676(0.7); 2.671(0.9); 2.667(0.7); 2.542(0.5); 2.525(2.4); 2.511(53.7); 2.507(107.1); 2.502(138.9); 2.498(98.9); 2.493(46.8);
2.474(8.5); 2.356(1.0); 2.334(0.8); 2.329(0.9); 2.324(0.7); 2.225(2.2); 2.164(16.0); 2.138(1.4); 2.116(1.3); 2.106(1.4); 2.085(2.3); 2.064(3.0); 2.045(2.8); 2.023(1.6); 2.004(0.8); 1.989(0.6); 1.974(0.4);
1.957(0.4); 1.918(0.6); 1.898(1.2); 1.876(1.4); 1.867(1.6); 1.848(0.9); 1.747(0.5); 1.741(0.6); 1.724(1.0); 1.700(0.9); 1.686(0.5); 1.679(0.5); 1.580(0.4); 1.550(0.4); 1.234(0.3); 0.146(1.1); 0.008(9.9);
0.000(259.6); −0.009(10.1); −0.150(1.1)
Example 308: ¹H-NMR(299.9 MHz, dmso):
δ = 7.877(0.9); 7.847(0.9); 7.627(1.2); 7.601(1.5); 7.569(0.6); 7.546(1.8); 7.533(1.1); 7.508(1.2); 7.483(0.5); 7.374(0.7); 7.349(1.1); 7.325(0.5); 4.586(0.5); 4.573(0.5); 4.559(0.5); 4.545(0.5); 3.426(0.5);
3.405(0.5); 3.387(0.5); 3.312(28.2); 2.558(15.2); 2.512(9.1); 2.506(19.0); 2.500(26.1); 2.494(18.6); 2.488(8.4); 2.196(0.5); 2.183(0.5); 2.169(0.5); 2.164(0.5); 2.138(0.4); 2.110(16.0); 2.064(0.5); 2.038(0.5);
1.941(0.5); 1.920(0.7); 1.900(0.9); 1.878(0.7); 1.861(0.5); 1.830(0.4); 1.804(0.5); 1.790(0.6); 1.777(0.4); 1.761(0.6); 1.746(0.4); 1.615(0.5); 1.607(0.4); 1.582(0.4); 0.010(0.7); −0.001(19.6); −0.012(0.6)
Example 309: ¹H-NMR(299.9 MHz, dmso):
δ = 14.794(0.5); 8.821(0.6); 8.792(0.6); 8.741(11.0); 8.728(10.7); 8.679(0.7); 8.610(0.6); 8.516(8.2); 8.485(8.2); 8.461(0.7); 7.964(0.6); 7.879(0.5); 7.729(9.9); 7.713(10.6); 7.702(14.5); 7.695(13.8);
7.687(15.2); 7.669(16.0); 7.623(15.8); 7.600(5.2); 7.504(0.6); 7.464(6.0); 7.441(9.1); 7.417(4.4); 7.372(0.9); 7.308(11.3); 7.285(9.9); 4.612(5.1); 4.600(4.2); 4.589(5.0); 4.568(2.5); 3.507(2.7); 3.478(4.7);
3.458(4.2); 3.440(5.1); 3.419(2.8); 3.380(1.5); 3.313(436.1); 3.247(1.6); 3.206(0.7); 3.177(2.1); 3.160(1.8); 3.111(0.6); 3.066(0.7); 3.042(0.6); 2.998(0.6); 2.970(0.6); 2.733(1.7); 2.727(2.5); 2.715(1.3);
2.629(0.6); 2.567(2.6); 2.513(146.5); 2.507(293.4); 2.501(397.2); 2.495(289.8); 2.489(140.9); 2.368(1.3); 2.318(1.0); 2.277(2.7); 2.271(3.5); 2.264(3.3); 2.228(3.6); 2.213(4.0); 2.202(4.6); 2.186(5.1);
2.177(4.0); 2.162(3.4); 2.134(2.8); 2.093(3.0); 2.061(4.6); 2.025(4.1); 1.974(3.3); 1.964(3.8); 1.949(7.4); 1.941(8.0); 1.923(8.3); 1.899(6.9); 1.761(2.8); 1.748(3.2); 1.737(4.9); 1.725(4.5); 1.715(6.5);
1.694(7.6); 1.685(7.8); 1.672(7.0); 1.643(3.6); 1.540(0.8); 1.523(0.7); 1.490(0.6); 1.448(0.6); 1.236(2.6); 0.854(0.7); 0.196(1.0); 0.066(0.5); 0.011(9.8); 0.000(243.0); −0.011(11.8); −0.056(1.1); −0.068(1.2); −0.199
(1.2)
Example 310: ¹H-NMR(299.9 MHz, dmso):
δ = 7.935(4.6); 7.594(1.5); 7.572(1.8); 7.554(1.8); 7.471(0.8); 7.444(2.2); 7.411(1.3); 7.320(1.0); 7.295(1.4); 7.270(0.6); 4.577(0.6); 4.554(0.6); 3.752(0.6); 3.730(15.5); 3.717(1.0); 3.630(0.4); 3.395(0.4);
3.372(0.7); 3.352(0.7); 3.313(50.1); 2.726(0.3); 2.513(17.4); 2.507(36.1); 2.500(49.7); 2.494(35.7); 2.488(16.5); 2.295(0.4); 2.281(0.4); 2.271(0.4); 2.196(0.4); 2.184(0.6); 2.171(0.6); 2.159(1.2); 2.145(0.6);
2.113(0.5); 2.073(0.5); 2.044(0.6); 1.974(16.0); 1.935(0.6); 1.917(0.7); 1.896(0.9); 1.869(0.6); 1.858(0.6); 1.835(0.4); 1.762(0.4); 1.750(0.5); 1.737(0.6); 1.724(0.5); 1.708(0.6); 1.693(0.4); 1.680(0.6);
1.653(0.4); 1.632(0.5); 1.593(0.5); 1.234(0.4); 0.011(1.6); 0.000(41.3); −0.011(1.4)
Example 311: ¹H-NMR(299.9 MHz, dmso):
δ = 8.351(4.3); 8.325(4.4); 7.426(0.6); 7.416(0.6); 7.410(1.0); 7.392(1.5); 7.386(2.8); 7.364(12.6); 7.359(16.0); 7.342(8.3); 7.336(7.9); 7.328(2.0); 7.315(3.2); 7.310(3.8); 7.307(3.7); 7.300(3.4); 7.285(2.5);
7.278(9.9); 7.270(6.1); 7.255(6.6); 7.252(9.5); 7.246(6.7); 7.230(5.5); 7.223(3.6); 7.214(0.7); 7.185(0.4); 7.056(0.7); 7.021(8.0); 6.992(12.2); 6.964(6.0); 6.925(0.4); 6.774(6.4); 6.772(6.3); 6.750(6.3);
6.745(6.0); 4.621(1.2); 4.595(3.7); 4.569(5.1); 4.543(3.5); 4.519(1.1); 3.677(2.1); 3.649(4.3); 3.642(2.9); 3.621(2.8); 3.614(4.5); 3.586(2.2); 3.381(3.9); 3.314(142.1); 3.247(4.1); 2.573(0.9); 2.567(1.2);
2.561(0.9); 2.536(0.4); 2.512(13.5); 2.506(28.7); 2.500(39.8); 2.494(29.5); 2.488(14.2); 2.440(1.0); 2.433(1.3); 2.428(1.0); 2.270(0.3); 2.190(0.6); 2.154(1.6); 2.120(3.4); 2.089(4.6); 2.054(3.8); 2.024(2.5);
1.992(3.4); 1.972(2.2); 1.950(2.3); 1.923(2.4); 1.915(2.4); 1.907(2.2); 1.899(2.0); 1.883(2.5); 1.876(2.8); 1.850(3.4); 1.824(2.6); 1.810(1.8); 1.795(2.8); 1.785(1.9); 1.777(1.8); 1.753(2.2); 1.728(1.1);
1.630(0.7); 1.597(1.5); 1.566(2.3); 1.534(2.1); 1.502(1.0); 1.236(0.7); 0.067(0.6); 0.011(0.9); 0.000(24.4); −0.011(1.0); −0.067(0.7)
Example 312: ¹H-NMR(299.9 MHz, dmso):
δ = 7.835(1.0); 7.809(1.0); 7.241(0.6); 7.235(0.5); 7.213(1.3); 7.207(0.5); 7.191(0.6); 7.186(0.8); 7.164(0.4); 6.958(1.7); 6.929(2.6); 6.900(1.3); 4.577(0.7); 4.551(0.9); 4.525(0.6); 3.644(0.4); 3.617(0.8);
3.582(0.8); 3.555(0.4); 3.313(49.9); 2.571(1.0); 2.541(15.2); 2.513(14.6); 2.507(29.8); 2.501(40.5); 2.495(29.0); 2.489(13.5); 2.329(0.8); 2.165(16.0); 2.108(0.6); 2.082(0.9); 2.051(0.8); 2.023(0.4);
1.999(0.4); 1.971(0.7); 1.949(0.7); 1.928(0.7); 1.906(0.6); 1.884(0.9); 1.864(1.1); 1.839(0.6); 1.822(0.4); 1.797(0.4); 1.554(0.4); 1.523(0.4); 0.011(1.5); 0.000(35.9); −0.011(1.3)
Example 313: ¹H-NMR(299.9 MHz, dmso):
δ = 8.760(0.7); 8.720(6.7); 8.707(6.9); 8.603(5.1); 8.576(5.3); 8.177(0.4); 8.149(0.5); 8.100(0.5); 8.073(0.5); 7.781(0.7); 7.762(0.7); 7.748(0.5); 7.735(0.6); 7.692(5.9); 7.676(5.8); 7.666(6.5); 7.650(6.2);
7.576(0.4); 7.549(0.5); 7.525(0.3); 7.365(1.6); 7.344(3.3); 7.338(2.9); 7.316(6.9); 7.310(3.0); 7.294(3.3); 7.288(4.5); 7.267(2.1); 7.124(7.4); 7.101(6.7); 7.065(9.9); 7.037(16.0); 7.008(7.6); 6.942(0.4);
6.639(1.2); 6.572(0.6); 5.755(0.7); 4.628(1.4); 4.603(3.9); 4.578(5.7); 4.553(3.9); 4.528(1.2); 3.680(2.2); 3.653(4.9); 3.645(3.3); 3.626(3.2); 3.618(4.9); 3.591(2.4); 3.508(0.4); 3.400(0.4); 3.379(0.7);
3.313(191.3); 3.270(0.5); 3.248(0.7); 3.039(0.4); 3.001(0.5); 2.992(0.5); 2.930(0.4); 2.733(1.3); 2.727(1.6); 2.721(1.1); 2.622(0.4); 2.568(1.5); 2.562(1.4); 2.513(85.4); 2.507(174.7); 2.501(236.7);
2.495(168.4); 2.489(77.7); 2.443(1.0); 2.435(1.0); 2.380(0.4); 2.368(0.3); 2.276(1.3); 2.271(1.5); 2.264(1.2); 2.203(0.6); 2.160(1.8); 2.135(3.5); 2.124(4.2); 2.098(5.1); 2.084(3.5); 2.069(3.6); 2.043(2.9);
2.014(3.5); 1.991(2.5); 1.945(2.3); 1.919(2.5); 1.882(2.9); 1.855(1.9); 1.800(1.8); 1.776(2.2); 1.745(3.0); 1.724(2.3); 1.702(2.7); 1.680(1.6); 1.656(1.0); 1.629(1.8); 1.596(2.7); 1.568(2.1); 1.533(1.1);
1.475(0.4); 1.236(1.0); 0.980(0.6); 0.196(0.9); 0.067(0.8); 0.011(8.9); 0.000(206.0); −0.011(6.9); −0.065(0.7); −0.199(0.8)
Example 314: ¹H-NMR(299.9 MHz, dmso):
δ = 14.684(0.4); 13.588(0.4); 12.270(0.4); 10.549(0.4); 8.794(0.5); 8.601(4.7); 8.574(4.9); 8.315(0.4); 8.177(0.6); 8.149(0.8); 8.100(0.7); 8.072(0.8); 7.756(0.4); 7.732(0.7); 7.708(0.4); 7.573(0.6);
7.549(0.7); 7.525(0.4); 7.451(2.1); 7.429(4.3); 7.423(3.5); 7.407(2.9); 7.401(8.3); 7.395(3.1); 7.378(3.4); 7.373(5.0); 7.351(2.4); 7.298(2.0); 7.276(4.0); 7.271(3.3); 7.255(2.7); 7.249(7.7); 7.242(3.2);
7.226(3.3); 7.221(4.9); 7.199(2.2); 7.184(0.4); 7.156(0.4); 7.124(0.8); 7.099(0.9); 7.070(0.7); 7.053(2.5); 7.043(13.2); 7.018(15.2); 6.990(11.5); 6.979(3.2); 6.968(10.3); 6.939(16.0); 6.911(8.1); 6.639(1.8);
6.391(0.4); 5.922(0.4); 4.645(1.4); 4.620(4.1); 4.594(6.0); 4.569(4.1); 4.545(1.4); 3.647(2.6); 3.619(5.3); 3.611(3.4); 3.592(3.4); 3.584(5.4); 3.557(2.5); 3.380(1.0); 3.313(438.2); 3.281(1.8); 3.247(1.4);
3.197(0.4); 3.180(0.6); 3.166(0.5); 3.102(0.4); 3.084(0.5); 3.069(0.4); 3.023(1.0); 2.982(0.7); 2.950(0.5); 2.892(0.4); 2.866(0.4); 2.855(0.5); 2.805(0.4); 2.763(0.4); 2.733(1.8); 2.727(2.5); 2.721(1.8);
2.678(0.4); 2.631(0.5); 2.599(0.5); 2.568(1.7); 2.537(3.4); 2.513(122.9); 2.507(257.2); 2.501(351.6); 2.495(246.8); 2.489(111.0); 2.441(1.3); 2.435(1.4); 2.362(0.5); 2.277(1.6); 2.270(2.2); 2.264(1.5);
2.187(0.7); 2.151(1.9); 2.111(4.5); 2.085(5.2); 2.074(3.8); 2.044(2.4); 2.018(1.8); 1.992(3.5); 1.972(2.6); 1.953(2.3); 1.928(2.2); 1.914(1.9); 1.905(2.4); 1.875(2.9); 1.867(3.1); 1.841(2.3); 1.802(1.9);
1.778(2.4); 1.772(2.5); 1.748(3.0); 1.738(2.0); 1.728(2.2); 1.706(2.7); 1.683(1.6); 1.636(1.0); 1.604(1.7); 1.574(2.5); 1.543(2.2); 1.508(1.1); 1.338(0.5); 1.235(1.8); 1.008(0.5); 0.997(0.4); 0.984(1.0);
0.974(0.5); 0.832(0.4); 0.196(1.3); 0.111(0.4); 0.067(0.9); 0.011(11.7); 0.000(318.3); −0.008(4.7); −0.011(8.9); −0.022(0.5); −0.039(0.4); −0.066(1.0); −0.199(1.2)
Example 315: ¹H-NMR(400.0 MHz, DMSO):
δ = 20.006(0.6); 8.441(0.5); 8.424(0.5); 8.379(7.9); 8.374(8.7); 8.367(8.7); 8.362(8.4); 8.340(4.9); 8.317(10.7); 7.967(0.4); 7.886(0.7); 7.752(0.6); 7.747(0.5); 7.728(1.0); 7.697(1.5); 7.678(3.1); 7.670(3.2);
7.659(7.1); 7.645(15.3); 7.640(13.7); 7.625(16.0); 7.597(3.1); 7.552(1.3); 7.532(1.1); 7.505(2.9); 7.488(9.5); 7.475(16.0); 7.455(11.9); 7.436(1.0); 7.410(0.7); 7.371(7.9); 7.359(7.9); 7.353(10.0); 7.341(9.8);
7.227(9.5); 7.222(9.9); 7.208(7.8); 7.203(7.5); 5.038(3.5); 4.824(0.8); 4.810(0.7); 4.714(1.2); 4.697(2.6); 4.685(2.8); 4.680(2.9); 4.675(2.9); 4.663(2.5); 4.645(1.2); 4.516(1.6); 4.504(1.6); 4.377(1.8);
4.366(1.9); 4.181(0.8); 4.170(0.9); 3.777(0.9); 3.697(0.6); 3.472(2.3); 3.452(2.0); 3.433(3.6); 3.409(3.6); 3.390(1.9); 3.323(1330.4); 3.120(1.0); 3.104(1.6); 3.093(1.6); 3.077(1.6); 3.063(0.9); 2.970(0.7);
2.942(0.4); 2.859(0.5); 2.675(10.7); 2.671(14.7); 2.666(10.5); 2.541(7.9); 2.524(46.4); 2.511(864.7); 2.506(1714.9); 2.502(2236.2); 2.497(1592.9); 2.493(753.7); 2.337(5.7); 2.333(11.4); 2.328(15.2);
2.324(11.1); 2.276(1.1); 2.164(2.0); 2.143(2.8); 2.131(2.9); 2.122(3.2); 2.113(3.3); 2.103(2.6); 2.091(2.9); 2.069(2.8); 2.059(4.1); 2.048(4.6); 2.038(7.0); 2.029(7.6); 2.010(6.6); 1.988(3.5); 1.958(3.2);
1.931(3.8); 1.920(4.4); 1.911(6.8); 1.901(5.8); 1.890(6.6); 1.883(5.8); 1.872(4.7); 1.859(4.1); 1.838(2.1); 1.799(2.1); 1.787(2.2); 1.769(1.6); 1.748(2.6); 1.730(3.7); 1.716(4.5); 1.697(6.4); 1.676(6.6);
1.655(4.8); 1.645(3.1); 1.635(3.4); 1.623(2.5); 1.599(1.9); 1.576(1.5); 1.558(1.2); 1.531(1.5); 1.511(1.6); 1.499(1.7); 1.479(1.6); 1.436(0.7); 1.418(0.6); 1.356(0.7); 1.337(0.8); 1.298(1.2); 1.258(1.8);
1.234(5.9); 1.099(0.5); 1.008(0.4); 0.854(1.3); 0.803(0.7); 0.798(0.8); 0.146(21.2); 0.008(208.6); 0.000(4806.4); −0.009(172.4); −0.021(10.3); −0.023(8.4); −0.024(7.7); −0.026(7.2); −0.028(5.6); −0.029(5.9); −0.033
(5.0); −0.034(5.0); −0.041(2.9); −0.044(3.5); −0.050(2.6); −0.055(2.4); −0.063(2.0); −0.068(1.9); −0.079(1.5); −0.088(1.5); −0.098(1.2); −0.150(21.7); −0.171(0.8); −0.194(0.7); −0.315(0.5); −0.353(0.4)
Example 316: ¹H-NMR(400.0 MHz, DMSO):
δ = 8.317(0.4); 8.147(5.4); 8.124(5.4); 7.418(0.4); 7.379(4.0); 7.375(5.4); 7.359(14.4); 7.355(14.5); 7.349(10.5); 7.345(10.9); 7.331(12.4); 7.328(16.0); 7.311(6.6); 7.308(7.5); 7.279(2.0); 7.275(1.9);
7.266(2.2); 7.261(5.1); 7.253(7.6); 7.248(8.9); 7.241(6.2); 7.234(10.7); 7.230(10.2); 7.223(3.2); 7.216(5.0); 7.212(4.6); 7.204(0.4); 7.151(5.8); 7.149(6.8); 7.133(9.0); 7.130(10.3); 7.114(8.7); 7.096(4.9);
7.093(5.0); 7.090(6.3); 7.087(5.3); 7.069(4.4); 7.067(4.0); 6.826(0.4); 6.804(0.3); 6.715(9.1); 6.711(9.1); 6.696(8.9); 6.692(8.5); 5.756(0.6); 4.712(1.3); 4.701(1.6); 4.694(3.0); 4.683(3.3); 4.677(3.2);
4.671(3.2); 4.661(2.9); 4.654(1.8); 4.643(1.4); 3.490(2.3); 3.472(4.8); 3.461(3.0); 3.455(3.1); 3.444(4.7); 3.427(2.3); 3.324(132.7); 2.674(0.7); 2.670(0.9); 2.665(0.7); 2.540(0.6); 2.523(2.5); 2.510(53.8);
2.505(108.5); 2.501(142.0); 2.496(100.2); 2.492(46.8); 2.332(0.6); 2.328(0.9); 2.323(0.6); 2.185(1.3); 2.173(1.2); 2.166(1.8); 2.161(1.8); 2.151(3.2); 2.142(3.1); 2.131(3.0); 2.123(3.4); 2.109(2.0);
2.100(3.1); 2.093(2.3); 2.068(3.7); 2.040(2.6); 2.019(1.2); 1.939(0.4); 1.921(2.8); 1.915(2.9); 1.898(6.5); 1.884(6.3); 1.877(4.5); 1.871(4.9); 1.868(5.1); 1.861(2.7); 1.853(2.5); 1.847(2.1); 1.840(1.0);
1.736(1.7); 1.725(1.8); 1.717(2.9); 1.705(3.8); 1.697(2.4); 1.685(3.8); 1.673(2.7); 1.666(3.1); 1.650(3.1); 1.642(1.8); 1.629(3.3); 1.623(2.8); 1.618(2.4); 1.610(1.7); 1.605(2.5); 1.599(2.4); 1.586(1.0);
1.575(1.3); 1.556(0.5); 1.398(0.4); 1.255(0.8); 1.249(0.9); 1.240(0.9); 1.234(1.1); 0.146(1.4); 0.008(11.0); 0.000(296.7); −0.009(9.9); −0.150(1.3)
Example 317: ¹H-NMR(400.0 MHz, DMSO):
δ = 8.048(2.1); 8.026(2.1); 7.391(1.7); 7.388(2.0); 7.371(4.4); 7.368(4.2); 7.352(2.1); 7.348(2.2); 7.334(2.7); 7.330(2.7); 7.314(1.3); 7.310(1.3); 7.223(1.9); 7.219(1.8); 7.204(3.1); 7.201(3.1); 7.186(1.6);
7.183(1.6); 7.176(0.4); 7.139(6.0); 7.117(6.8); 6.832(1.3); 6.825(7.7); 6.804(6.7); 6.796(0.7); 6.728(2.9); 6.724(2.9); 6.709(2.8); 6.705(2.6); 4.599(0.5); 4.588(1.2); 4.581(1.2); 4.573(2.7); 4.569(1.5);
4.558(3.9); 4.550(1.2); 4.543(2.9); 4.528(1.3); 3.325(69.0); 3.268(0.8); 3.246(1.6); 3.227(1.6); 3.206(0.8); 2.670(0.4); 2.523(1.2); 2.510(23.5); 2.505(46.5); 2.501(59.9); 2.496(42.1); 2.492(19.6); 2.328(0.4);
2.092(0.5); 2.074(0.7); 2.069(0.7); 2.059(1.1); 2.050(1.1); 2.041(1.1); 2.037(0.9); 2.031(0.9); 2.019(0.6); 1.985(1.5); 1.963(3.2); 1.950(2.1); 1.930(1.0); 1.924(1.0); 1.885(0.5); 1.877(0.7); 1.867(0.9);
1.854(0.8); 1.839(1.0); 1.821(0.7); 1.710(0.6); 1.696(0.6); 1.690(1.0); 1.677(1.4); 1.671(0.8); 1.664(0.7); 1.658(1.4); 1.645(1.2); 1.635(1.4); 1.632(1.5); 1.615(1.3); 1.609(0.8); 1.596(0.7); 1.587(0.9);
1.566(0.6); 1.398(5.5); 1.270(0.4); 1.255(15.4); 1.249(16.0); 1.240(15.5); 1.234(15.4); 0.146(0.5); 0.008(4.5); 0.000(111.9); −0.009(3.9); −0.150(0.5)
Example 318: ¹H-NMR(400.0 MHz, DMSO):
δ = 8.272(1.9); 8.250(2.0); 7.968(2.3); 7.962(1.5); 7.956(2.5); 7.951(1.5); 7.945(2.7); 7.935(0.4); 7.620(0.6); 7.615(0.3); 7.607(4.5); 7.601(2.6); 7.596(3.8); 7.595(3.7); 7.589(2.6); 7.585(3.7); 7.583(3.5);
7.570(0.7); 7.178(0.4); 7.155(1.0); 7.137(5.7); 7.116(6.2); 6.856(1.3); 6.847(1.0); 6.840(7.6); 6.835(3.4); 6.818(6.5); 6.808(2.7); 6.801(1.6); 6.797(2.5); 6.791(1.7); 6.785(2.4); 6.776(0.3); 4.589(0.9);
4.574(2.6); 4.559(3.6); 4.543(3.2); 4.534(1.2); 4.528(1.4); 4.521(1.0); 4.504(0.4); 3.324(16.6); 3.269(0.7); 3.250(1.3); 3.227(1.3); 3.208(0.7); 2.670(0.3); 2.524(0.7); 2.510(19.2); 2.506(39.4); 2.501(51.7);
2.497(36.6); 2.492(17.1); 2.328(0.4); 2.089(0.5); 2.070(0.7); 2.066(0.7); 2.056(0.9); 2.046(0.9); 2.037(1.0); 2.028(0.9); 2.016(0.8); 2.005(1.0); 1.993(1.4); 1.984(1.5); 1.972(2.0); 1.956(0.9); 1.947(1.4);
1.926(0.8); 1.906(0.6); 1.891(0.6); 1.884(0.8); 1.875(1.1); 1.865(1.0); 1.854(0.9); 1.845(1.0); 1.835(0.7); 1.825(0.7); 1.814(0.4); 1.724(0.6); 1.711(0.6); 1.705(0.9); 1.692(1.2); 1.686(0.8); 1.674(1.6);
1.660(1.8); 1.643(1.4); 1.637(0.9); 1.615(0.9); 1.604(0.4); 1.596(0.6); 1.300(0.5); 1.284(0.6); 1.271(0.7); 1.255(7.5); 1.250(15.9); 1.243(15.6); 1.235(16.0); 1.228(13.9); 0.146(0.5); 0.008(4.6); 0.000(120.5);
−0.009(4.2); −0.150(0.5)
Example 319: ¹H-NMR(400.0 MHz, DMSO):
δ = 8.316(0.6); 8.140(4.7); 8.118(4.7); 7.554(0.4); 7.540(7.2); 7.537(5.2); 7.534(4.5); 7.522(7.9); 7.518(8.2); 7.503(0.4); 7.352(2.9); 7.348(3.3); 7.333(6.2); 7.329(6.7); 7.314(3.6); 7.310(3.7); 7.295(1.8);
7.291(2.7); 7.283(2.1); 7.277(8.1); 7.273(8.3); 7.266(11.2); 7.260(16.0); 7.255(8.4); 7.247(11.0); 7.242(8.7); 7.228(4.2); 7.223(2.1); 7.160(5.0); 7.157(5.7); 7.141(7.6); 7.138(8.5); 7.121(7.9); 7.101(4.0);
7.095(5.3); 7.092(4.5); 7.074(3.7); 7.072(3.3); 6.655(0.3); 6.641(7.4); 6.635(7.2); 6.623(5.0); 6.618(6.9); 5.756(0.7); 4.704(1.1); 4.693(1.4); 4.686(2.6); 4.676(2.8); 4.670(2.7); 4.664(2.8); 4.653(2.5);
4.647(1.5); 4.635(1.2); 3.858(0.4); 3.486(2.0); 3.468(4.1); 3.457(2.6); 3.451(2.7); 3.440(4.0); 3.423(2.0); 3.323(202.0); 2.675(1.3); 2.670(1.7); 2.666(1.2); 2.540(1.1); 2.523(4.5); 2.510(99.4); 2.506(198.4);
2.501(258.2); 2.497(183.5); 2.492(86.6); 2.332(1.2); 2.328(1.6); 2.323(1.2); 2.184(1.0); 2.172(1.2); 2.165(1.3); 2.160(1.5); 2.151(2.7); 2.141(2.7); 2.130(3.0); 2.107(2.8); 2.099(2.8); 2.074(3.4); 2.054(2.2);
2.045(2.3); 2.024(1.1); 1.919(2.0); 1.912(2.8); 1.895(6.5); 1.884(4.5); 1.880(4.9); 1.868(4.8); 1.849(2.3); 1.748(1.4); 1.737(1.6); 1.729(2.3); 1.717(3.0); 1.709(1.9); 1.697(3.2); 1.686(2.2); 1.677(2.0);
1.668(2.3); 1.651(2.1); 1.644(1.3); 1.631(2.9); 1.620(2.0); 1.612(1.6); 1.606(2.0); 1.601(2.2); 1.588(0.8); 1.580(1.0); 1.559(0.4); 0.146(1.8); 0.008(14.2); 0.000(372.8); −0.009(13.1); −0.150(1.8)
Example 320: ¹H-NMR(299.9 MHz, dmso):
δ = 8.745(0.4); 8.716(0.4); 8.582(5.3); 8.551(5.4); 7.732(0.4); 7.708(0.9); 7.656(7.7); 7.631(9.1); 7.585(3.8); 7.560(10.9); 7.546(6.4); 7.522(7.4); 7.496(3.1); 7.483(1.9); 7.460(4.0); 7.455(3.5); 7.432(7.1);
7.426(3.2); 7.405(8.5); 7.382(8.7); 7.357(3.0); 7.095(2.6); 7.085(11.4); 7.059(16.0); 7.032(8.6); 7.021(1.8); 4.638(1.8); 4.615(3.8); 4.604(2.9); 4.593(3.7); 4.572(1.6); 4.561(1.2); 3.457(1.7); 3.435(3.4);
3.415(3.1); 3.398(3.3); 3.376(1.8); 3.314(80.4); 3.293(1.3); 2.727(0.4); 2.506(40.2); 2.500(48.8); 2.494(33.7); 2.252(1.2); 2.220(2.3); 2.207(2.6); 2.194(3.1); 2.178(3.3); 2.152(2.3); 2.127(1.3); 2.079(1.8);
2.047(3.4); 2.010(2.5); 1.984(2.5); 1.954(3.5); 1.923(6.2); 1.903(5.7); 1.888(4.2); 1.861(2.4); 1.817(0.6); 1.792(0.5); 1.764(1.8); 1.753(2.0); 1.741(2.6); 1.731(3.1); 1.709(3.9); 1.687(6.4); 1.667(4.3);
1.637(2.2); 1.606(1.1); 1.576(0.5); 0.010(1.8); −0.001(24.9); −0.011(1.0)
Example 321: ¹H-NMR(299.9 MHz, dmso):
δ = 8.532(5.2); 8.506(5.4); 8.461(0.4); 8.434(0.7); 8.427(0.6); 8.418(0.6); 8.411(0.6); 8.388(7.5); 8.382(8.4); 8.372(8.5); 8.365(8.4); 8.148(0.3); 8.072(0.4); 7.713(0.4); 7.706(0.6); 7.688(0.6); 7.681(0.5);
7.548(0.3); 7.468(0.6); 7.452(0.7); 7.443(0.6); 7.427(0.5); 7.406(8.6); 7.390(8.5); 7.381(11.2); 7.365(10.7); 7.334(1.8); 7.312(3.8); 7.307(3.4); 7.285(7.4); 7.279(3.5); 7.262(3.7); 7.257(5.0); 7.236(2.4);
7.197(10.0); 7.191(10.7); 7.172(8.3); 7.166(8.2); 7.121(0.3); 7.102(0.4); 7.076(0.4); 7.060(0.8); 7.030(10.4); 7.001(16.0); 6.973(7.7); 6.931(0.5); 6.638(1.0); 6.571(0.5); 5.920(0.4); 5.754(0.8); 4.645(0.5);
4.624(1.5); 4.599(4.7); 4.573(6.8); 4.548(4.6); 4.522(1.5); 3.685(2.7); 3.658(5.7); 3.651(3.9); 3.630(3.8); 3.623(5.9); 3.596(2.8); 3.383(1.6); 3.312(77.5); 3.283(1.2); 3.243(1.3); 3.031(0.6); 3.018(0.5);
2.996(0.6); 2.972(0.4); 2.726(0.4); 2.572(0.9); 2.512(14.2); 2.506(28.4); 2.500(38.5); 2.494(28.6); 2.489(13.9); 2.423(0.9); 2.270(0.4); 2.186(0.9); 2.151(3.2); 2.146(3.3); 2.116(6.0); 2.085(6.5); 2.050(3.5);
2.031(2.4); 2.006(4.3); 1.984(3.1); 1.942(2.5); 1.929(2.4); 1.920(2.9); 1.913(2.8); 1.905(2.8); 1.889(3.1); 1.882(3.6); 1.857(2.6); 1.830(2.6); 1.806(2.9); 1.801(2.9); 1.789(2.1); 1.776(3.6); 1.767(2.4);
1.757(2.4); 1.734(2.8); 1.710(1.5); 1.686(0.4); 1.647(1.1); 1.613(2.0); 1.585(3.2); 1.553(2.6); 1.521(1.2); 1.491(0.3); 1.235(0.7); 1.000(0.5); 0.976(1.0); 0.952(0.5); 0.010(1.4); 0.000(26.4)
Example 322: ¹H-NMR(299.9 MHz, dmso):
δ = 8.299(6.3); 8.273(6.5); 8.177(0.5); 8.149(0.4); 8.101(0.5); 8.073(0.5); 7.824(0.4); 7.789(9.1); 7.763(9.6); 7.732(0.7); 7.705(0.6); 7.693(0.5); 7.577(0.4); 7.548(0.6); 7.525(0.5); 7.405(0.5); 7.373(0.5);
7.323(5.2); 7.309(4.1); 7.301(12.1); 7.298(12.3); 7.282(7.9); 7.276(9.8); 7.259(4.2); 7.254(5.2); 7.233(2.2); 7.145(0.5); 7.120(0.7); 7.098(5.1); 7.093(6.1); 7.073(7.8); 7.067(9.5); 7.047(5.3); 7.042(6.0);
7.031(10.1); 7.002(16.0); 6.974(7.7); 6.639(1.3); 6.605(7.5); 6.600(9.1); 6.580(7.6); 6.574(9.1); 6.341(0.4); 4.610(0.4); 4.599(1.4); 4.573(4.3); 4.548(6.2); 4.523(4.2); 4.499(1.4); 3.665(2.4); 3.637(5.2);
3.602(5.7); 3.575(2.6); 3.414(0.4); 3.405(0.4); 3.385(2.0); 3.316(153.0); 3.244(2.1); 3.024(0.3); 2.727(0.6); 2.571(1.3); 2.507(45.3); 2.501(64.5); 2.495(54.5); 2.425(1.1); 2.271(0.7); 2.203(0.8); 2.165(2.2);
2.127(4.0); 2.098(5.9); 2.062(5.3); 2.026(2.6); 2.014(2.8); 1.988(4.7); 1.947(4.4); 1.922(4.5); 1.885(4.3); 1.862(4.9); 1.844(3.9); 1.830(2.9); 1.816(3.9); 1.775(2.7); 1.750(1.2); 1.635(0.9); 1.604(2.3);
1.571(3.2); 1.541(2.9); 1.510(1.5); 1.236(3.7); 0.983(0.4); 0.854(0.5); 0.073(0.5); 0.000(39.7); −0.071(0.3)
Example 323: ¹H-NMR(299.9 MHz, dmso):
δ = 8.425(5.6); 8.399(5.6); 8.177(0.3); 8.149(0.4); 8.073(0.4); 7.822(0.4); 7.759(0.6); 7.731(0.9); 7.692(5.2); 7.670(6.9); 7.662(7.4); 7.645(1.1); 7.602(2.4); 7.583(7.6); 7.575(8.4); 7.563(16.0); 7.553(7.3);
7.523(1.5); 7.354(1.6); 7.332(3.4); 7.326(3.2); 7.304(6.5); 7.283(3.3); 7.277(4.2); 7.255(2.0); 7.099(0.4); 7.054(9.2); 7.025(15.0); 6.997(7.0); 6.769(0.6); 6.751(5.6); 6.744(6.0); 6.723(5.6); 6.639(0.9);
6.573(1.0); 4.633(0.4); 4.616(1.4); 4.591(4.4); 4.565(6.4); 4.540(4.4); 4.515(1.3); 3.671(2.6); 3.643(5.3); 3.636(3.5); 3.616(3.5); 3.609(5.4); 3.581(2.6); 3.385(1.7); 3.314(119.6); 3.245(1.4); 3.013(0.4);
2.995(0.5); 2.726(0.4); 2.576(0.9); 2.507(34.6); 2.501(45.5); 2.495(32.7); 2.431(0.8); 2.271(0.5); 2.212(0.7); 2.177(2.0); 2.142(3.0); 2.111(4.2); 2.079(3.9); 2.050(3.4); 2.042(3.6); 2.026(3.3); 2.004(4.0);
1.982(2.9); 1.963(2.5); 1.940(2.5); 1.924(2.1); 1.917(2.7); 1.901(2.6); 1.878(3.3); 1.852(2.3); 1.825(2.4); 1.800(2.8); 1.783(1.9); 1.770(3.3); 1.760(2.3); 1.752(2.2); 1.728(2.7); 1.704(1.4); 1.639(0.9);
1.606(1.9); 1.576(2.9); 1.544(2.5); 1.516(1.1); 1.481(0.4); 1.236(0.6); 1.000(0.3); 0.976(0.7); 0.952(0.3); 0.010(1.5); 0.000(26.9); −0.010(1.1)
Example 324: ¹H-NMR(299.9 MHz, dmso):
δ = 7.841(4.9); 7.378(1.3); 7.352(1.3); 7.207(0.3); 7.186(0.8); 7.179(0.7); 7.158(1.4); 7.130(0.9); 7.109(0.4); 6.903(2.1); 6.874(3.3); 6.846(1.6); 4.568(0.9); 4.543(1.3); 4.518(0.9); 3.729(1.3); 3.699(15.7);
3.600(0.5); 3.574(1.0); 3.539(1.0); 3.512(0.5); 3.313(30.3); 2.506(32.6); 2.500(42.4); 2.494(31.1); 2.183(0.4); 2.166(1.3); 2.118(0.9); 2.087(1.3); 2.053(1.1); 2.006(16.0); 1.974(0.7); 1.948(1.1); 1.928(1.3);
1.910(1.1); 1.841(0.5); 1.816(0.7); 1.784(0.8); 1.742(0.5); 1.597(0.4); 1.564(0.6); 1.530(0.5); −0.001(29.0)
Example 325: ¹H-NMR(400.0 MHz, DMSO):
δ = 8.115(2.0); 8.093(2.0); 7.808(0.4); 7.766(0.3); 7.743(0.4); 7.720(0.4); 7.687(1.9); 7.683(2.3); 7.665(2.6); 7.559(0.8); 7.541(2.1); 7.525(3.3); 7.520(3.3); 7.506(2.2); 7.487(0.8); 7.168(0.4); 7.151(5.5);
7.130(6.4); 6.860(0.8); 6.853(7.2); 6.842(0.9); 6.831(6.2); 6.663(2.1); 6.646(2.1); 4.608(1.1); 4.593(2.6); 4.578(3.6); 4.563(2.6); 4.554(1.0); 4.548(1.4); 4.536(0.5); 3.324(55.3); 3.267(0.8); 3.245(1.6);
3.226(1.6); 3.204(0.7); 2.675(0.5); 2.670(0.6); 2.666(0.5); 2.541(0.4); 2.524(1.7); 2.510(38.5); 2.506(77.4); 2.501(100.6); 2.497(70.7); 2.492(32.8); 2.333(0.5); 2.328(0.7); 2.324(0.5); 2.084(0.5); 2.062(0.8);
2.052(0.9); 2.042(1.0); 2.033(1.1); 2.024(0.8); 2.012(0.6); 1.992(1.2); 1.978(1.8); 1.971(2.4); 1.957(2.6); 1.950(1.8); 1.936(1.4); 1.870(0.5); 1.856(0.8); 1.846(1.1); 1.831(0.7); 1.817(0.8); 1.679(0.5);
1.666(0.7); 1.660(1.0); 1.647(1.7); 1.641(0.9); 1.629(2.4); 1.616(1.8); 1.609(1.6); 1.596(0.6); 1.589(0.9); 1.570(0.4); 1.284(0.4); 1.266(14.2); 1.258(14.9); 1.251(16.0); 1.243(14.1); 1.236(2.2); 1.159(0.5);
1.120(0.4); 0.982(0.3); 0.146(0.9); 0.008(7.9); 0.000(201.5); −0.009(6.8); −0.150(0.9)
Example 326: ¹H-NMR(400.0 MHz, DMSO):
δ = 8.379(2.9); 8.374(3.0); 8.367(3.1); 8.362(3.0); 8.316(0.4); 8.222(2.0); 8.200(2.0); 7.360(2.8); 7.348(2.7); 7.342(3.3); 7.330(3.3); 7.171(3.2); 7.166(3.3); 7.152(2.8); 7.147(2.8); 7.136(5.7); 7.115(6.4);
6.830(7.3); 6.813(2.5); 6.809(6.3); 6.801(0.6); 4.599(0.5); 4.588(1.3); 4.582(1.1); 4.573(2.8); 4.565(1.3); 4.558(3.6); 4.543(2.6); 4.528(1.0); 3.323(60.4); 3.271(0.6); 3.252(1.3); 3.228(1.4); 3.209(0.7);
2.675(0.4); 2.670(0.5); 2.666(0.4); 2.524(1.4); 2.510(31.4); 2.506(62.3); 2.501(80.6); 2.497(56.8); 2.492(26.4); 2.333(0.4); 2.328(0.5); 2.324(0.4); 2.106(0.5); 2.084(0.8); 2.074(0.9); 2.065(1.0); 2.055(1.1);
2.046(0.8); 2.034(0.6); 2.016(0.3); 2.000(0.4); 1.989(1.2); 1.979(1.4); 1.969(1.4); 1.959(2.0); 1.933(1.3); 1.912(0.8); 1.880(0.8); 1.869(0.9); 1.860(1.0); 1.850(0.7); 1.841(0.8); 1.831(0.6); 1.822(0.4);
1.708(0.5); 1.695(0.6); 1.689(0.9); 1.676(1.2); 1.669(1.2); 1.657(1.5); 1.649(1.9); 1.637(1.7); 1.617(0.7); 1.611(0.8); 1.590(0.5); 1.252(14.7); 1.246(16.0); 1.237(15.1); 1.231(15.5); 0.008(2.2); 0.000(54.9); −0.009
(1.8)
Example 327: ¹H-NMR(400.0 MHz, DMSO):
δ = 8.043(2.2); 8.021(2.2); 7.552(2.5); 7.545(1.6); 7.540(2.0); 7.534(1.7); 7.529(2.9); 7.521(0.4); 7.270(0.8); 7.258(3.5); 7.255(3.7); 7.251(3.2); 7.245(4.9); 7.238(3.0); 7.233(4.6); 7.220(0.7); 7.214(0.3);
7.202(0.4); 7.180(0.4); 7.143(6.1); 7.121(6.9); 6.831(7.7); 6.809(6.7); 6.664(2.7); 6.658(1.7); 6.652(2.6); 6.649(1.7); 6.641(2.5); 4.591(1.1); 4.576(3.0); 4.561(4.2); 4.546(3.0); 4.531(1.0); 4.526(0.6);
3.326(100.2); 3.263(0.8); 3.241(1.8); 3.223(1.8); 3.201(0.8); 2.670(0.5); 2.665(0.4); 2.510(28.6); 2.506(56.8); 2.501(74.0); 2.497(53.1); 2.492(25.6); 2.332(0.3); 2.328(0.4); 2.324(0.3); 2.091(0.5);
2.073(0.7); 2.068(0.7); 2.058(1.2); 2.048(1.1); 2.039(1.1); 2.030(1.0); 2.017(0.6); 2.010(0.5); 1.988(2.0); 1.970(2.0); 1.964(3.2); 1.950(2.8); 1.942(2.0); 1.927(1.3); 1.887(0.5); 1.878(0.6); 1.869(0.9);
1.855(1.0); 1.839(1.0); 1.823(0.7); 1.723(0.6); 1.709(0.7); 1.703(1.0); 1.690(1.4); 1.683(0.8); 1.677(0.7); 1.671(1.4); 1.657(1.1); 1.652(1.1); 1.638(1.4); 1.617(1.4); 1.610(0.9); 1.598(0.8); 1.588(1.0);
1.568(0.6); 1.398(2.1); 1.257(15.4); 1.250(16.0); 1.242(15.9); 1.235(15.5); 1.192(0.4); 1.175(0.7); 1.157(0.4); 0.008(1.8); 0.000(48.8); −0.009(1.8)
Example 328: ¹H-NMR(400.0 MHz, DMSO):
δ = 8.411(5.1); 8.389(5.2); 8.316(0.6); 7.439(1.9); 7.422(4.1); 7.418(3.9); 7.406(2.8); 7.401(7.9); 7.397(3.1); 7.385(4.0); 7.380(4.7); 7.364(2.2); 7.308(2.7); 7.304(3.7); 7.285(7.6); 7.269(3.9); 7.266(4.2);
7.244(1.8); 7.240(1.8); 7.231(2.4); 7.225(5.1); 7.211(4.3); 7.206(5.6); 7.201(3.1); 7.192(3.3); 7.188(2.7); 7.174(0.4); 7.089(9.4); 7.070(10.9); 7.063(5.5); 7.060(7.1); 7.057(6.2); 7.054(6.5); 7.051(5.9);
7.041(14.9); 7.023(16.0); 7.021(16.0); 7.002(11.1); 6.994(1.9); 4.717(1.2); 4.708(1.5); 4.700(2.7); 4.692(3.3); 4.685(2.9); 4.678(3.4); 4.670(2.7); 4.661(1.6); 4.652(1.2); 3.469(0.4); 3.458(2.3); 3.441(4.5);
3.429(3.0); 3.424(3.1); 3.413(4.5); 3.395(2.3); 3.325(100.3); 3.024(0.9); 2.856(0.7); 2.675(0.5); 2.670(0.8); 2.666(0.6); 2.523(1.8); 2.510(46.5); 2.506(95.4); 2.501(126.3); 2.497(91.6); 2.492(44.5);
2.332(0.6); 2.328(0.8); 2.323(0.6); 2.182(1.2); 2.160(2.4); 2.148(2.7); 2.141(3.1); 2.130(3.2); 2.122(2.3); 2.111(1.6); 2.090(0.9); 2.076(2.2); 2.069(1.8); 2.046(3.9); 2.017(2.6); 1.994(1.3); 1.940(1.5);
1.933(2.0); 1.921(3.8); 1.915(4.4); 1.903(5.2); 1.897(4.6); 1.887(5.3); 1.874(4.1); 1.859(2.7); 1.843(1.3); 1.834(1.0); 1.701(2.2); 1.691(2.0); 1.684(4.1); 1.674(3.5); 1.665(5.6); 1.653(5.2); 1.644(7.7);
1.633(3.3); 1.625(2.5); 1.603(1.1); 1.583(0.5); 1.398(0.3); 1.235(0.4); 1.162(0.3); 1.144(0.7); 1.005(0.6); 0.146(0.4); 0.008(3.0); 0.000(94.3); −0.009(3.5); −0.150(0.4)
Example 329: ¹H-NMR(400.0 MHz, DMSO):
δ = 8.334(1.0); 8.313(1.0); 7.432(0.6); 7.427(0.6); 7.411(1.1); 7.394(0.6); 7.390(0.7); 7.115(2.8); 7.094(3.2); 7.064(0.3); 7.056(1.8); 7.037(2.4); 7.035(2.4); 7.016(1.6); 6.777(3.5); 6.755(3.1); 4.579(0.5);
4.563(1.0); 4.548(1.4); 4.533(1.5); 4.518(1.0); 4.503(0.4); 3.325(64.1); 3.229(0.3); 3.212(0.7); 3.189(0.6); 3.171(0.4); 2.670(0.3); 2.510(21.2); 2.506(42.2); 2.501(55.3); 2.497(39.9); 2.492(19.2); 2.328(0.3);
2.045(0.3); 2.037(0.5); 2.029(0.5); 2.025(0.5); 2.020(0.5); 2.016(0.5); 2.008(0.7); 1.998(0.5); 1.988(0.7); 1.978(0.9); 1.969(0.7); 1.958(0.8); 1.935(0.6); 1.912(0.4); 1.886(0.3); 1.863(0.4); 1.851(0.4);
1.842(0.5); 1.833(0.4); 1.671(0.6); 1.652(0.8); 1.643(0.9); 1.633(0.9); 1.623(0.5); 1.614(0.5); 1.602(0.3); 1.248(1.6); 1.240(16.0); 1.233(2.3); 1.225(15.7); 0.008(1.6); 0.000(39.3); −0.009(1.4)
Example 330: ¹H-NMR(400.0 MHz, DMSO):
δ = 8.979(0.4); 8.973(0.5); 8.923(10.2); 8.917(12.2); 8.874(12.7); 8.868(9.9); 8.595(0.4); 8.535(0.4); 8.528(0.4); 8.499(4.9); 8.476(4.9); 8.317(0.5); 8.225(0.7); 8.218(0.7); 8.112(0.9); 8.106(0.7); 7.660(0.4);
7.639(0.7); 7.600(11.6); 7.580(16.0); 7.537(0.5); 7.517(0.4); 7.478(15.1); 7.457(11.2); 5.758(3.0); 4.751(1.1); 4.741(1.5); 4.734(2.5); 4.725(3.0); 4.718(2.8); 4.711(3.0); 4.703(2.4); 4.695(1.5); 4.686(1.1);
4.254(0.5); 4.233(0.4); 4.038(0.4); 4.020(0.5); 3.429(1.8); 3.411(3.2); 3.404(2.7); 3.386(3.4); 3.368(2.0); 3.326(208.5); 2.676(1.1); 2.671(1.5); 2.667(1.2); 2.511(95.7); 2.507(188.9); 2.502(245.6);
2.498(174.7); 2.493(82.8); 2.333(1.1); 2.329(1.6); 2.324(1.1); 2.169(0.9); 2.149(2.1); 2.141(2.2); 2.133(2.6); 2.128(2.6); 2.119(2.7); 2.111(2.4); 2.095(2.1); 2.079(2.9); 2.068(2.9); 2.059(4.3); 2.048(3.8);
2.042(4.7); 2.030(2.7); 2.021(2.7); 2.016(3.3); 1.995(2.1); 1.989(3.2); 1.965(0.9); 1.946(0.9); 1.925(1.4); 1.917(2.2); 1.904(2.7); 1.895(3.1); 1.881(2.0); 1.861(1.0); 1.853(0.9); 1.777(0.8); 1.762(2.6);
1.757(2.2); 1.752(2.3); 1.747(3.2); 1.741(4.1); 1.737(4.1); 1.730(3.8); 1.719(6.3); 1.705(4.1); 1.699(2.9); 1.681(1.4); 1.661(0.4); 1.523(0.3); 1.506(0.8); 1.489(0.8); 1.298(0.3); 1.259(0.5); 1.235(2.5);
1.193(0.6); 1.175(1.1); 1.157(0.6); 0.895(3.3); 0.879(3.1); 0.854(0.3); 0.146(1.5); 0.030(0.4); 0.008(15.1); 0.000(336.6); −0.009(12.0); −0.028(0.4); −0.150(1.6)
Example 331: ¹H-NMR(400.0 MHz, DMSO):
δ = 8.709(7.2); 8.699(7.4); 8.385(5.2); 8.363(5.4); 8.317(0.6); 7.671(12.9); 7.650(16.0); 7.630(5.7); 7.623(6.2); 7.611(5.5); 7.579(0.4); 7.482(14.9); 7.462(12.7); 7.149(7.4); 7.129(6.8); 5.757(0.3);
4.724(1.1); 4.705(2.8); 4.695(3.3); 4.689(3.2); 4.684(3.3); 4.673(2.8); 4.656(1.1); 3.450(1.9); 3.431(3.9); 3.408(4.2); 3.389(2.1); 3.326(152.6); 2.671(1.4); 2.502(228.6); 2.329(1.5); 2.153(1.1); 2.135(2.1);
2.123(3.0); 2.117(2.9); 2.106(3.2); 2.096(2.7); 2.079(2.2); 2.064(3.3); 2.054(3.6); 2.035(6.8); 2.014(5.4); 1.992(2.2); 1.961(0.8); 1.939(1.1); 1.921(1.5); 1.911(2.5); 1.900(3.0); 1.891(3.3); 1.881(2.3);
1.872(2.0); 1.854(1.3); 1.739(0.7); 1.722(2.7); 1.701(4.4); 1.693(5.0); 1.680(6.7); 1.665(4.3); 1.654(2.6); 1.642(1.6); 1.622(0.4); 1.235(1.4); 0.146(1.1); 0.000(238.2); −0.150(1.2)
Example 332: ¹H-NMR(400.0 MHz, DMSO):
δ = 8.316(0.6); 8.092(8.0); 8.070(8.1); 7.872(0.4); 7.854(0.4); 7.775(14.3); 7.756(14.8); 7.452(0.6); 7.434(0.4); 7.353(5.0); 7.334(10.4); 7.319(5.6); 7.315(5.9); 7.295(6.8); 7.278(16.0); 7.265(7.6);
7.260(13.3); 7.252(6.7); 7.246(7.9); 7.232(4.4); 7.228(3.8); 7.197(0.6); 7.191(0.5); 7.162(9.2); 7.144(13.5); 7.124(6.5); 7.120(8.4); 7.094(8.8); 7.081(7.6); 7.076(9.2); 7.061(11.8); 7.058(12.1); 7.042(5.9);
7.038(5.9); 6.534(11.6); 6.530(11.9); 6.515(11.4); 6.511(11.1); 5.756(14.5); 4.688(1.7); 4.677(2.4); 4.670(4.1); 4.660(4.7); 4.654(4.7); 4.648(4.7); 4.638(4.2); 4.631(2.6); 4.620(1.9); 3.850(0.5); 3.486(3.1);
3.469(6.4); 3.457(4.3); 3.452(4.4); 3.440(6.4); 3.423(3.1); 3.324(202.0); 2.674(1.4); 2.670(1.9); 2.666(1.4); 2.540(0.9); 2.505(224.7); 2.501(293.9); 2.497(219.0); 2.332(1.5); 2.328(2.0); 2.181(1.6);
2.170(2.0); 2.163(2.2); 2.156(2.8); 2.148(4.4); 2.138(5.0); 2.128(4.6); 2.112(5.2); 2.106(5.3); 2.095(3.3); 2.080(5.8); 2.058(3.9); 2.051(4.2); 2.029(2.3); 1.961(0.3); 1.933(1.6); 1.911(6.1); 1.894(9.4);
1.884(8.1); 1.880(8.2); 1.868(6.8); 1.849(3.5); 1.781(2.5); 1.770(2.8); 1.762(3.9); 1.750(4.9); 1.742(3.4); 1.730(5.3); 1.719(3.6); 1.710(2.5); 1.699(2.5); 1.678(1.8); 1.656(3.3); 1.649(2.1); 1.635(4.6);
1.625(3.3); 1.617(2.9); 1.611(3.3); 1.605(3.9); 1.592(1.5); 1.581(1.8); 1.562(0.7); 1.234(0.3); 1.194(0.7); 1.176(1.4); 1.158(0.7); 1.012(0.7); 0.994(1.4); 0.976(0.7); 0.876(0.5); 0.000(1.4)
Example 333: ¹H-NMR(400.0 MHz, DMSO):
δ = 7.995(2.3); 7.972(2.3); 7.786(3.4); 7.767(3.5); 7.264(1.6); 7.246(3.4); 7.227(2.0); 7.211(0.4); 7.190(0.4); 7.145(6.0); 7.124(6.9); 7.084(1.6); 7.080(1.8); 7.064(2.6); 7.061(2.8); 7.045(1.3); 7.042(1.3);
6.859(0.4); 6.835(7.3); 6.813(6.2); 6.571(2.6); 6.567(2.8); 6.552(2.6); 6.548(2.6); 5.756(2.2); 4.591(0.9); 4.585(0.7); 4.576(2.3); 4.561(3.4); 4.546(3.2); 4.532(1.9); 4.515(0.6); 3.324(62.2); 3.262(0.8);
3.240(1.8); 3.222(1.8); 3.200(0.8); 2.670(0.6); 2.505(69.0); 2.501(90.3); 2.497(68.6); 2.328(0.6); 2.086(0.5); 2.074(0.6); 2.069(0.6); 2.063(0.7); 2.054(1.1); 2.044(1.1); 2.035(1.1); 2.026(0.9); 2.013(0.6);
2.003(0.7); 1.990(1.2); 1.975(2.1); 1.969(2.9); 1.954(3.4); 1.932(1.7); 1.904(0.5); 1.893(0.6); 1.875(0.9); 1.861(1.1); 1.844(1.0); 1.831(0.8); 1.816(0.4); 1.754(0.7); 1.740(0.8); 1.734(1.0); 1.721(1.4);
1.714(0.8); 1.708(0.7); 1.702(1.4); 1.689(0.9); 1.683(0.7); 1.670(0.7); 1.663(0.5); 1.644(1.1); 1.623(1.3); 1.603(0.9); 1.593(1.0); 1.582(0.4); 1.573(0.6); 1.300(0.3); 1.285(0.6); 1.270(0.7); 1.256(15.2);
1.248(15.5); 1.241(16.0); 1.233(14.8); 0.000(0.4)
Example 334: ¹H-NMR(400.0 MHz, DMSO):
δ = 8.601(2.0); 8.597(2.2); 8.589(2.2); 8.585(2.2); 8.567(3.0); 8.562(2.8); 8.047(1.5); 8.025(1.5); 7.810(1.1); 7.805(1.8); 7.800(1.1); 7.790(1.3); 7.785(1.9); 7.780(1.2); 7.391(1.6); 7.379(1.5); 7.372(1.4);
7.371(1.4); 7.360(1.3); 7.359(1.4); 7.174(0.3); 7.103(4.3); 7.081(4.8); 6.806(0.3); 6.784(0.3); 6.752(0.6); 6.745(5.2); 6.724(4.7); 4.595(0.9); 4.576(1.1); 4.557(0.9); 4.539(0.3); 4.507(0.6); 4.492(1.5);
4.477(2.0); 4.462(1.5); 4.447(0.6); 4.056(0.7); 4.038(2.0); 4.020(2.0); 4.002(0.7); 3.326(11.1); 3.295(0.5); 3.276(1.3); 3.254(1.2); 3.234(0.6); 2.511(7.6); 2.506(15.5); 2.502(20.5); 2.497(14.9); 2.493(7.3);
2.089(0.4); 2.076(0.5); 2.067(0.9); 2.057(0.8); 2.048(1.0); 2.036(1.0); 2.025(0.7); 2.013(1.6); 2.004(0.8); 1.989(9.8); 1.971(1.2); 1.965(0.9); 1.947(0.3); 1.941(0.5); 1.929(0.4); 1.920(0.8); 1.911(0.7);
1.901(0.7); 1.891(0.8); 1.881(0.5); 1.870(0.4); 1.767(0.5); 1.751(0.5); 1.746(0.8); 1.730(0.9); 1.726(0.6); 1.718(0.5); 1.713(0.7); 1.710(0.7); 1.697(0.6); 1.694(0.5); 1.678(0.5); 1.650(0.7); 1.643(0.3);
1.629(0.9); 1.621(0.7); 1.608(0.6); 1.599(0.8); 1.578(0.5); 1.222(1.5); 1.207(1.5); 1.193(2.4); 1.175(6.3); 1.169(16.0); 1.154(15.9); 0.008(1.1); 0.000(30.0); −0.009(1.2)
Example 335: ¹H-NMR(400.0 MHz, DMSO):
δ = 8.597(3.2); 8.591(3.8); 8.535(3.8); 8.529(3.1); 8.332(1.1); 8.309(1.1); 7.147(3.2); 7.125(3.6); 6.794(0.5); 6.787(4.0); 6.765(3.6); 6.758(0.4); 4.619(0.5); 4.610(0.6); 4.604(0.6); 4.597(0.6); 4.588(0.5);
4.581(0.3); 4.559(0.5); 4.544(1.2); 4.529(1.6); 4.514(1.2); 4.499(0.5); 3.323(20.7); 3.260(0.4); 3.242(0.7); 3.235(0.5); 3.225(0.6); 3.217(0.7); 3.199(0.4); 2.510(12.9); 2.506(26.1); 2.501(34.4); 2.497(24.7);
2.493(11.9); 2.090(0.4); 2.081(0.4); 2.073(0.5); 2.069(0.5); 2.060(0.5); 2.052(0.5); 2.041(0.5); 2.029(0.4); 2.021(0.6); 2.010(0.5); 2.000(0.6); 1.992(0.6); 1.980(0.5); 1.961(0.6); 1.936(0.6); 1.914(0.4);
1.878(0.4); 1.871(0.4); 1.858(0.5); 1.850(0.5); 1.837(0.4); 1.737(0.3); 1.722(0.6); 1.711(0.6); 1.702(1.0); 1.692(0.9); 1.681(1.1); 1.670(0.6); 1.243(1.0); 1.233(16.0); 1.218(15.5); 0.008(1.4); 0.000(41.2); −0.009
(1.6)
Example 336: ¹H-NMR(400.0 MHz, DMSO):
δ = 8.611(0.4); 8.598(13.7); 8.592(15.9); 8.530(16.0); 8.524(13.2); 8.438(4.0); 8.415(4.0); 8.316(0.4); 7.329(2.9); 7.310(5.9); 7.291(3.3); 7.245(1.4); 7.240(1.4); 7.231(1.7); 7.226(3.3); 7.223(3.2);
7.212(3.4); 7.207(4.3); 7.202(2.5); 7.192(2.4); 7.188(2.0); 7.100(5.6); 7.095(5.7); 7.075(13.6); 7.055(7.0); 5.756(1.2); 4.749(1.0); 4.740(1.2); 4.732(2.3); 4.724(2.9); 4.717(2.4); 4.708(2.9); 4.701(2.2);
4.693(1.3); 4.684(1.0); 3.487(1.8); 3.470(3.6); 3.458(2.6); 3.453(2.5); 3.441(3.7); 3.424(1.9); 3.324(110.8); 2.675(0.7); 2.671(0.9); 2.666(0.7); 2.510(56.2); 2.506(110.0); 2.502(143.0); 2.497(103.1);
2.333(0.7); 2.328(0.9); 2.324(0.7); 2.219(1.2); 2.201(1.4); 2.195(1.9); 2.186(2.0); 2.175(2.4); 2.163(2.4); 2.158(2.0); 2.145(1.6); 2.125(0.8); 2.115(0.8); 2.092(1.6); 2.084(1.7); 2.061(3.3); 2.039(2.1);
2.032(2.5); 2.010(1.3); 1.989(1.1); 1.969(1.3); 1.962(1.4); 1.950(2.8); 1.944(2.7); 1.939(2.1); 1.932(3.0); 1.925(2.0); 1.914(3.6); 1.899(3.4); 1.883(1.9); 1.875(2.1); 1.869(2.3); 1.854(0.9); 1.848(1.1);
1.841(0.9); 1.761(1.5); 1.751(1.6); 1.744(2.0); 1.734(2.5); 1.729(1.9); 1.723(2.3); 1.714(3.4); 1.701(4.6); 1.699(4.6); 1.683(3.1); 1.660(1.8); 1.644(0.7); 1.638(0.9); 1.614(0.4); 1.235(0.9); 1.175(0.5);
0.146(0.8); 0.008(7.3); 0.000(162.0); −0.008(6.5); −0.150(0.8)
Example 337: ¹H-NMR(400.0 MHz, DMSO):
δ = 8.317(0.5); 7.736(3.4); 7.714(3.5); 7.589(9.9); 7.577(10.4); 7.377(2.3); 7.361(4.3); 7.358(4.6); 7.342(2.5); 7.339(2.5); 7.237(1.0); 7.233(1.0); 7.218(2.8); 7.214(2.4); 7.199(3.3); 7.185(1.7); 7.181(1.5);
7.109(4.2); 7.102(11.0); 7.090(16.0); 7.072(2.6); 7.065(3.0); 7.059(3.8); 7.038(2.5); 6.574(0.4); 5.756(3.9); 4.667(0.8); 4.649(1.9); 4.634(2.2); 4.617(2.0); 4.599(0.8); 4.056(0.4); 4.038(1.3); 4.020(1.3);
4.003(0.4); 3.509(1.4); 3.491(2.7); 3.481(1.8); 3.474(1.9); 3.464(2.8); 3.446(1.3); 3.324(56.3); 2.675(0.5); 2.671(0.7); 2.667(0.5); 2.506(79.1); 2.502(103.6); 2.498(76.5); 2.333(0.5); 2.329(0.7); 2.325(0.5);
2.189(0.7); 2.178(0.9); 2.171(0.9); 2.165(1.2); 2.155(1.9); 2.146(2.4); 2.137(1.9); 2.131(1.8); 2.124(2.5); 2.115(2.2); 2.102(1.8); 2.092(2.4); 2.065(2.0); 2.043(0.8); 1.989(5.5); 1.962(1.8); 1.944(3.7);
1.931(3.5); 1.917(3.8); 1.899(2.0); 1.879(0.6); 1.804(1.1); 1.792(1.2); 1.785(1.7); 1.772(2.2); 1.764(1.5); 1.752(2.3); 1.740(1.4); 1.732(1.1); 1.721(1.1); 1.709(0.6); 1.690(1.4); 1.681(0.9); 1.667(2.1);
1.657(1.5); 1.648(1.2); 1.643(1.4); 1.638(1.6); 1.624(0.6); 1.617(0.8); 1.398(3.0); 1.193(1.5); 1.175(2.8); 1.157(1.4); 0.147(0.6); 0.008(5.1); 0.000(129.4); −0.149(0.6)
Example 338: ¹H-NMR(400.0 MHz, DMSO):
δ = 7.611(2.9); 7.598(3.0); 7.564(1.0); 7.544(1.0); 7.143(2.9); 7.122(3.5); 7.117(3.6); 7.104(3.0); 6.797(0.6); 6.791(3.7); 6.769(3.3); 5.756(1.6); 4.548(0.5); 4.533(1.2); 4.518(1.7); 4.512(0.8); 4.503(1.6);
4.488(0.8); 3.323(16.3); 3.286(0.4); 3.266(0.7); 3.245(0.7); 3.225(0.4); 2.524(0.6); 2.510(13.3); 2.506(26.7); 2.502(35.0); 2.497(25.1); 2.493(12.2); 2.077(0.3); 2.068(0.6); 2.058(0.5); 2.049(0.5); 2.044(0.5);
2.039(0.5); 2.026(0.8); 2.014(0.6); 2.004(1.4); 1.989(0.7); 1.983(1.0); 1.979(1.0); 1.959(0.5); 1.926(0.3); 1.917(0.3); 1.908(0.5); 1.898(0.5); 1.889(0.4); 1.878(0.5); 1.771(0.3); 1.758(0.4); 1.753(0.5);
1.739(0.7); 1.733(0.4); 1.720(0.6); 1.707(0.5); 1.701(0.5); 1.687(0.7); 1.667(0.6); 1.639(0.4); 1.398(0.4); 1.237(1.0); 1.218(16.0); 1.203(15.7); 0.008(1.8); 0.000(47.2); −0.009(1.9)
Example 339: ¹H-NMR(400.0 MHz, DMSO):
δ = 8.599(9.0); 8.595(9.7); 8.587(9.5); 8.583(9.5); 8.552(13.3); 8.546(13.3); 8.317(0.5); 8.164(5.7); 8.142(5.8); 7.795(5.1); 7.790(8.3); 7.785(5.3); 7.775(5.7); 7.770(9.2); 7.766(5.6); 7.394(7.5); 7.382(7.5);
7.375(7.1); 7.363(7.1); 7.357(4.0); 7.353(4.5); 7.338(7.5); 7.334(8.2); 7.319(4.2); 7.315(4.3); 7.265(0.5); 7.251(0.4); 7.246(0.6); 7.241(0.3); 7.231(0.3); 7.185(1.7); 7.181(1.9); 7.171(2.1); 7.166(5.5);
7.161(4.4); 7.147(7.3); 7.142(3.5); 7.133(3.4); 7.128(3.7); 7.103(0.4); 7.101(0.4); 7.063(7.0); 7.044(11.4); 7.038(7.0); 7.028(4.2); 7.026(4.6); 7.018(4.9); 7.012(6.6); 7.009(5.7); 6.991(4.3); 6.989(4.1);
5.757(5.1); 4.733(1.4); 4.715(3.5); 4.697(4.2); 4.680(3.6); 4.661(1.5); 4.056(1.1); 4.038(3.5); 4.020(3.5); 4.002(1.2); 3.538(2.6); 3.520(5.4); 3.510(3.2); 3.502(3.3); 3.493(5.1); 3.475(2.6); 3.444(0.5);
3.326(61.8); 3.201(0.4); 3.187(0.4); 3.173(0.4); 2.676(0.5); 2.671(0.6); 2.667(0.5); 2.524(1.8); 2.511(38.1); 2.507(77.5); 2.502(103.0); 2.498(75.1); 2.494(37.0); 2.333(0.5); 2.329(0.7); 2.325(0.5);
2.185(1.3); 2.176(1.6); 2.166(2.5); 2.162(2.4); 2.153(3.5); 2.143(5.2); 2.134(4.8); 2.130(3.7); 2.121(5.2); 2.111(5.5); 2.102(2.7); 2.093(3.4); 2.085(2.8); 2.072(0.8); 2.064(2.1); 2.050(0.4); 2.043(0.4);
1.989(16.0); 1.975(1.4); 1.968(1.9); 1.960(3.4); 1.953(4.6); 1.950(4.5); 1.944(3.6); 1.933(8.4); 1.923(4.7); 1.917(5.0); 1.905(4.1); 1.886(1.8); 1.881(1.5); 1.871(0.4); 1.863(0.4); 1.848(0.3); 1.840(0.4);
1.833(0.4); 1.826(0.4); 1.818(0.4); 1.803(2.0); 1.789(2.0); 1.782(3.9); 1.769(4.8); 1.762(2.8); 1.757(2.2); 1.749(4.8); 1.736(2.9); 1.729(2.2); 1.716(2.1); 1.693(1.4); 1.671(2.6); 1.664(1.6); 1.650(3.8);
1.641(2.8); 1.627(2.6); 1.619(2.9); 1.606(1.2); 1.595(1.8); 1.583(0.4); 1.574(0.7); 1.478(0.4); 1.462(0.3); 1.447(0.4); 1.397(2.2); 1.235(0.5); 1.193(4.0); 1.175(7.9); 1.157(3.9); 0.146(0.7); 0.008(5.5);
0.000(143.2); −0.008(5.7); −0.150(0.7)
Example 340: ¹H-NMR(400.0 MHz, DMSO):
δ = 8.919(2.1); 8.914(2.5); 8.873(2.6); 8.868(2.0); 8.334(1.1); 8.312(1.1); 7.144(3.2); 7.122(3.6); 6.792(0.5); 6.785(4.0); 6.764(3.6); 6.756(0.4); 4.615(0.3); 4.609(0.5); 4.600(0.7); 4.593(0.6); 4.587(0.7);
4.578(0.6); 4.571(0.4); 4.562(0.7); 4.547(1.2); 4.532(1.6); 4.517(1.2); 4.502(0.5); 3.326(41.5); 3.243(0.4); 3.225(0.7); 3.219(0.6); 3.209(0.6); 3.202(0.7); 3.183(0.4); 2.510(15.0); 2.506(29.3); 2.502(38.1);
2.497(27.4); 2.493(13.2); 2.073(0.4); 2.064(0.5); 2.057(0.7); 2.050(0.6); 2.042(0.7); 2.035(0.7); 2.024(0.7); 2.015(0.7); 2.004(0.8); 1.995(0.6); 1.984(0.5); 1.964(0.6); 1.941(0.6); 1.919(0.4); 1.890(0.4);
1.863(0.5); 1.850(0.5); 1.841(0.6); 1.827(0.4); 1.737(0.4); 1.727(0.6); 1.724(0.6); 1.713(1.0); 1.704(0.9); 1.693(1.3); 1.681(0.7); 1.672(0.7); 1.246(0.9); 1.232(16.0); 1.217(15.2); 1.207(0.9); 0.909(0.6);
0.892(0.6); 0.008(1.1); 0.000(27.1); −0.008(1.0)
Example 341: ¹H-NMR(400.0 MHz, DMSO):
δ = 8.919(12.7); 8.913(15.6); 8.881(0.6); 8.869(16.0); 8.863(12.7); 8.598(0.4); 8.592(0.4); 8.530(0.5); 8.524(0.4); 8.447(5.6); 8.424(5.6); 8.316(0.7); 8.226(0.8); 8.219(0.9); 8.113(0.9); 8.106(0.8);
7.342(3.7); 7.339(4.0); 7.324(7.4); 7.320(8.0); 7.303(4.3); 7.301(4.4); 7.250(1.9); 7.245(1.9); 7.236(2.3); 7.231(5.0); 7.217(4.9); 7.211(5.8); 7.207(3.5); 7.197(3.4); 7.193(3.1); 7.104(6.8); 7.087(10.7);
7.080(7.7); 7.077(7.6); 7.070(11.7); 7.059(5.5); 7.056(5.8); 7.052(5.5); 7.038(0.5); 5.756(0.7); 4.736(1.4); 4.728(1.7); 4.719(3.1); 4.712(4.1); 4.704(3.3); 4.696(4.1); 4.689(2.9); 4.681(1.8); 4.672(1.4);
4.258(0.7); 4.241(0.7); 3.483(2.6); 3.466(4.9); 3.454(3.7); 3.450(3.6); 3.438(5.1); 3.421(2.8); 3.327(112.8); 2.675(0.6); 2.671(0.9); 2.667(0.6); 2.541(0.5); 2.524(2.4); 2.511(49.2); 2.506(98.6); 2.502(128.8);
2.497(91.4); 2.493(43.1); 2.333(0.6); 2.329(0.8); 2.324(0.6); 2.207(1.5); 2.201(1.1); 2.183(2.6); 2.174(2.6); 2.165(3.0); 2.162(3.1); 2.157(2.7); 2.151(3.7); 2.145(2.7); 2.132(2.3); 2.123(1.5); 2.113(1.3);
2.100(2.3); 2.092(2.3); 2.069(4.6); 2.047(2.9); 2.040(3.5); 2.018(1.9); 1.989(0.6); 1.981(1.8); 1.973(1.9); 1.961(3.9); 1.955(3.9); 1.942(4.0); 1.936(2.9); 1.930(3.1); 1.924(3.3); 1.907(3.7); 1.890(3.1);
1.884(3.4); 1.877(2.7); 1.869(3.0); 1.864(3.1); 1.856(2.1); 1.849(1.5); 1.841(1.5); 1.834(1.2); 1.759(2.1); 1.750(2.3); 1.744(3.4); 1.735(3.3); 1.724(4.2); 1.711(7.1); 1.702(4.7); 1.693(6.3); 1.673(2.7);
1.664(2.1); 1.658(1.1); 1.650(1.3); 1.643(0.9); 1.627(0.5); 1.539(0.4); 1.522(1.0); 1.505(0.9); 1.235(1.1); 0.905(3.8); 0.888(3.7); 0.146(0.5); 0.008(3.9); 0.000(108.0); −0.009(3.7); −0.150(0.5)
Example 342: ¹H-NMR(400.0 MHz, DMSO):
δ = 8.482(1.0); 8.460(1.0); 7.089(2.8); 7.068(3.3); 6.826(0.7); 6.819(3.7); 6.802(1.2); 6.798(3.1); 4.590(0.4); 4.575(1.0); 4.560(1.4); 4.545(1.1); 4.530(0.4); 4.505(0.4); 4.494(0.5); 4.489(0.5); 4.484(0.5);
4.473(0.4); 3.330(36.1); 3.206(0.6); 3.183(0.6); 3.164(0.4); 2.692(0.9); 2.648(14.5); 2.524(0.5); 2.511(11.2); 2.506(22.6); 2.502(29.5); 2.497(21.0); 2.493(9.9); 2.067(0.3); 2.057(0.5); 2.047(0.5); 2.039(0.5);
2.029(0.4); 1.989(0.6); 1.954(0.5); 1.943(0.5); 1.934(1.1); 1.925(0.8); 1.914(0.8); 1.909(0.8); 1.889(0.5); 1.858(0.5); 1.848(0.5); 1.839(0.5); 1.830(0.4); 1.651(0.5); 1.639(0.4); 1.632(0.8); 1.622(0.8);
1.613(1.0); 1.594(0.7); 1.582(0.4); 1.254(16.0); 1.239(15.9); 1.230(1.3); 0.000(7.8)
Example 343: ¹H-NMR(400.0 MHz, DMSO):
δ = 8.567(1.1); 8.545(1.1); 7.289(0.7); 7.282(0.5); 7.270(1.9); 7.263(1.2); 7.257(1.0); 7.250(1.6); 7.244(1.0); 7.230(0.6); 7.225(0.4); 7.161(1.3); 7.142(1.8); 7.124(0.7); 7.109(1.0); 7.082(1.1); 7.062(0.8);
4.603(0.6); 4.590(0.7); 3.478(0.4); 3.461(0.9); 3.450(0.6); 3.444(0.6); 3.433(0.9); 3.415(0.4); 3.329(45.1); 2.670(0.4); 2.650(16.0); 2.506(35.4); 2.502(46.0); 2.498(34.2); 2.150(0.4); 2.141(0.6); 2.131(0.6);
2.121(0.7); 2.112(0.5); 2.051(0.4); 2.021(0.7); 1.999(0.5); 1.993(0.5); 1.921(0.4); 1.914(0.5); 1.897(1.2); 1.889(1.0); 1.881(1.2); 1.846(0.4); 1.669(0.6); 1.661(0.6); 1.650(1.1); 1.641(1.0); 1.630(1.4);
1.610(0.8); 0.008(0.8); 0.000(17.3)
Example 344: ¹H-NMR(299.9 MHz, dmso):
δ = 7.682(1.2); 7.652(1.3); 7.564(0.4); 7.536(2.2); 7.510(2.1); 7.427(0.4); 7.363(1.5); 7.338(2.3); 7.292(1.2); 7.267(1.9); 7.242(0.9); 7.126(1.0); 7.122(1.2); 7.101(1.6); 7.099(1.6); 7.096(1.6); 7.077(0.7);
7.075(0.7); 7.072(0.7); 4.764(0.3); 4.753(0.6); 4.740(0.7); 4.724(0.7); 4.712(0.6); 4.701(0.4); 3.531(0.5); 3.509(1.2); 3.486(0.8); 3.470(1.0); 3.448(0.5); 3.323(40.5); 2.665(0.5); 2.601(0.6); 2.564(2.7);
2.537(14.8); 2.507(14.5); 2.501(20.7); 2.495(16.3); 2.342(2.7); 2.204(0.4); 2.186(0.6); 2.175(0.8); 2.161(0.8); 2.142(1.1); 2.115(1.0); 2.107(1.2); 2.082(16.0); 2.043(0.5); 1.954(0.4); 1.919(0.6); 1.891(1.0);
1.868(1.3); 1.847(0.9); 1.829(0.8); 1.807(0.6); 1.768(0.6); 1.754(0.5); 1.742(0.8); 1.727(0.8); 1.714(0.7); 1.699(0.8); 1.684(0.6); 1.671(0.6); 1.640(0.5); 1.616(0.6); 1.584(0.5); 1.234(0.7); 0.010(0.6);
0.007(0.6); 0.000(16.0); −0.003(11.3); −0.011(0.9)
Example 345: ¹H-NMR(299.9 MHz, dmso):
δ = 7.962(1.1); 7.832(4.5); 7.753(0.3); 7.559(0.4); 7.555(0.4); 7.532(0.5); 7.528(0.5); 7.507(1.6); 7.503(1.7); 7.480(1.9); 7.476(1.9); 7.439(0.3); 7.419(0.5); 7.413(0.5); 7.375(1.3); 7.370(1.3); 7.349(2.0);
7.344(1.8); 7.325(0.5); 7.239(1.2); 7.229(1.4); 7.218(1.9); 7.214(1.9); 7.193(1.6); 7.105(0.4); 7.079(1.2); 7.074(1.2); 7.053(1.4); 7.048(1.4); 7.028(0.8); 7.023(0.7); 4.711(0.6); 4.700(0.5); 4.690(0.6);
4.668(0.3); 3.711(4.5); 3.703(16.0); 3.625(0.6); 3.509(0.5); 3.487(1.0); 3.466(0.9); 3.448(0.9); 3.428(0.6); 3.325(30.9); 2.514(9.5); 2.508(19.7); 2.502(27.1); 2.496(20.2); 2.490(9.8); 2.272(0.4); 2.224(3.7);
2.188(0.7); 2.166(0.5); 2.151(0.8); 2.140(1.0); 2.127(0.9); 2.107(1.0); 2.098(0.9); 2.072(0.7); 2.041(0.4); 2.003(15.2); 1.936(0.6); 1.930(0.6); 1.916(0.5); 1.891(0.8); 1.869(1.1); 1.847(0.7); 1.831(0.6);
1.806(0.5); 1.788(0.4); 1.716(0.4); 1.704(0.6); 1.691(0.6); 1.675(0.8); 1.661(0.9); 1.649(1.3); 1.634(0.9); 1.628(0.8); 1.602(0.5); 1.596(0.5); 1.235(0.4); 0.011(1.0); 0.000(25.0); −0.011(1.0)
Example 346: ¹H-NMR(299.9 MHz, dmso):
δ = 8.761(0.9); 8.741(0.7); 8.716(5.1); 8.703(5.1); 8.700(5.1); 8.363(4.1); 8.331(4.2); 7.804(0.3); 7.783(0.7); 7.753(0.7); 7.737(0.6); 7.711(0.4); 7.684(4.5); 7.668(4.6); 7.658(5.2); 7.642(4.9); 7.604(7.8);
7.578(7.3); 7.576(7.0); 7.467(0.5); 7.447(0.8); 7.441(0.7); 7.399(1.8); 7.385(15.4); 7.383(16.0); 7.373(10.9); 7.367(9.2); 7.364(7.6); 7.341(1.4); 7.338(1.3); 7.226(0.6); 7.212(3.9); 7.200(3.3); 7.193(3.4);
7.185(4.3); 7.182(4.2); 7.175(3.4); 7.166(3.1); 7.155(3.3); 7.139(5.6); 7.136(5.8); 7.114(5.0); 7.110(5.0); 4.844(0.9); 4.835(1.2); 4.812(2.6); 4.799(1.8); 4.789(2.7); 4.767(1.2); 4.758(0.9); 4.568(0.4);
4.539(0.3); 3.552(1.9); 3.530(3.7); 3.509(2.9); 3.490(3.6); 3.469(1.8); 3.425(0.4); 3.399(0.6); 3.366(0.5); 3.324(98.0); 2.728(0.5); 2.721(0.4); 2.514(22.6); 2.508(45.1); 2.502(60.6); 2.495(42.9); 2.490(19.2);
2.271(0.7); 2.265(0.8); 2.244(1.0); 2.218(1.8); 2.205(2.1); 2.192(2.5); 2.179(2.6); 2.167(2.0); 2.142(2.3); 2.101(2.5); 2.071(1.6); 2.060(1.6); 2.032(0.9); 1.950(0.8); 1.911(2.8); 1.891(4.7); 1.871(3.3);
1.854(2.6); 1.833(1.5); 1.800(0.7); 1.772(0.5); 1.730(0.7); 1.707(2.0); 1.678(3.7); 1.668(3.4); 1.652(4.7); 1.634(2.7); 1.624(2.3); 1.610(1.0); 1.600(0.9); 1.236(2.2); 0.011(2.7); 0.000(59.7); −0.011(1.7)
Example 347: ¹H-NMR(299.9 MHz, dmso):
δ = 8.432(5.5); 8.401(5.4); 7.557(8.4); 7.553(8.3); 7.530(9.8); 7.526(9.1); 7.493(0.4); 7.460(2.0); 7.438(4.4); 7.432(3.7); 7.416(3.3); 7.410(8.1); 7.404(3.1); 7.388(4.0); 7.382(5.0); 7.357(5.7); 7.351(5.6);
7.331(9.7); 7.325(9.2); 7.285(5.3); 7.282(5.3); 7.261(8.7); 7.235(4.1); 7.146(5.8); 7.140(5.6); 7.120(7.5); 7.116(7.2); 7.096(4.1); 7.090(3.7); 7.069(2.4); 7.058(12.7); 7.033(16.0); 7.005(10.0); 6.995(1.9);
4.838(1.5); 4.828(1.8); 4.807(4.0); 4.794(2.7); 4.784(4.0); 4.762(1.7); 4.753(1.4); 3.507(2.8); 3.485(4.8); 3.465(4.9); 3.446(4.6); 3.424(2.2); 3.323(152.0); 2.727(0.7); 2.513(35.0); 2.507(68.0); 2.501(89.8);
2.495(64.0); 2.489(29.4); 2.271(0.8); 2.245(1.1); 2.218(2.3); 2.201(2.5); 2.192(3.2); 2.176(3.2); 2.167(2.7); 2.149(1.9); 2.134(2.8); 2.125(2.3); 2.104(2.1); 2.093(4.0); 2.064(2.8); 2.053(1.9); 2.023(1.3);
1.951(1.1); 1.923(2.1); 1.887(5.1); 1.867(6.4); 1.845(4.6); 1.829(2.9); 1.804(1.6); 1.714(1.9); 1.703(3.1); 1.693(3.4); 1.665(4.9); 1.648(5.9); 1.627(3.7); 1.568(0.3); 1.237(0.8); 0.011(4.2); 0.000(80.3); −0.011
(2.6)
Example 348: ¹H-NMR(299.9 MHz, dmso):
δ = 8.661(0.3); 8.430(0.5); 8.423(0.5); 8.414(0.6); 8.407(0.7); 8.388(5.6); 8.382(6.0); 8.372(6.0); 8.366(5.8); 8.311(4.0); 8.280(4.1); 7.732(0.4); 7.726(0.4); 7.707(0.5); 7.701(0.5); 7.581(6.5); 7.556(7.0);
7.553(6.7); 7.491(0.4); 7.486(0.4); 7.461(1.0); 7.446(0.8); 7.437(0.8); 7.421(0.7); 7.401(6.0); 7.385(7.2); 7.376(9.8); 7.359(16.0); 7.351(13.9); 7.333(6.2); 7.330(6.1); 7.307(1.9); 7.304(1.8); 7.185(9.1);
7.180(8.8); 7.161(9.0); 7.155(9.5); 7.135(2.7); 7.127(2.4); 4.836(0.9); 4.826(1.2); 4.813(2.1); 4.804(2.7); 4.792(1.9); 4.781(2.8); 4.760(1.2); 4.749(1.0); 4.581(0.3); 4.575(0.4); 4.552(0.3); 3.547(1.8);
3.525(3.7); 3.505(3.1); 3.486(3.5); 3.464(1.7); 3.435(0.3); 3.404(0.3); 3.375(0.4); 3.323(70.3); 2.727(0.3); 2.513(16.1); 2.507(32.0); 2.501(43.4); 2.496(32.1); 2.490(15.7); 2.271(0.5); 2.256(1.1); 2.224(1.7);
2.210(1.9); 2.198(2.4); 2.182(2.5); 2.172(1.9); 2.155(1.8); 2.130(1.9); 2.094(2.8); 2.067(1.8); 2.054(1.7); 2.026(1.1); 1.963(0.5); 1.910(2.8); 1.899(2.5); 1.882(4.8); 1.861(2.9); 1.844(2.7); 1.821(1.4);
1.776(0.5); 1.754(1.3); 1.742(1.3); 1.730(2.1); 1.719(2.2); 1.703(2.8); 1.694(2.5); 1.687(2.9); 1.677(4.6); 1.656(3.1); 1.623(1.6); 1.599(0.8); 1.234(1.4); 1.143(0.9); 1.106(0.8); 1.060(0.5); 1.039(0.5);
0.859(0.5); 0.011(1.6); 0.000(35.1); −0.010(1.7)
Example 349: ¹H-NMR(299.9 MHz, dmso):
δ = 8.492(0.4); 8.464(0.4); 8.156(0.5); 8.125(1.8); 8.093(1.5); 8.053(0.6); 7.710(0.5); 7.554(2.8); 7.551(2.6); 7.528(3.3); 7.525(2.9); 7.502(0.4); 7.466(0.4); 7.461(0.5); 7.440(0.7); 7.435(0.7); 7.410(0.4);
7.380(1.6); 7.371(1.8); 7.364(2.3); 7.352(3.4); 7.344(5.0); 7.337(6.1); 7.315(3.0); 7.309(4.3); 7.288(1.3); 7.283(1.8); 7.247(1.6); 7.241(1.5); 7.232(1.0); 7.223(2.2); 7.217(2.1); 7.209(0.8); 7.200(1.2);
7.194(1.1); 7.157(1.4); 7.151(1.4); 7.131(1.9); 7.127(1.8); 7.108(1.3); 7.101(1.0); 7.091(0.4); 6.718(2.1); 6.715(2.1); 6.694(2.0); 6.689(1.9); 6.616(1.3); 4.817(0.4); 4.806(0.4); 4.793(0.8); 4.783(1.0);
4.772(0.8); 4.761(1.0); 4.740(0.5); 4.728(0.4); 4.567(0.4); 3.519(0.6); 3.497(1.3); 3.476(1.1); 3.457(1.2); 3.435(0.7); 3.419(0.4); 3.390(0.4); 3.299(17.7); 2.483(12.2); 2.477(16.0); 2.471(12.1); 2.236(0.3);
2.219(0.5); 2.189(0.7); 2.174(0.9); 2.162(1.1); 2.148(1.1); 2.135(1.1); 2.120(1.2); 2.105(0.8); 2.089(1.3); 2.061(0.8); 2.049(0.6); 2.021(0.4); 1.884(0.8); 1.861(1.1); 1.842(1.7); 1.821(1.4); 1.804(1.0);
1.773(0.8); 1.745(0.5); 1.727(0.6); 1.716(0.6); 1.704(0.9); 1.691(0.9); 1.677(1.0); 1.665(1.0); 1.660(1.0); 1.648(1.1); 1.635(1.5); 1.611(1.0); 1.599(0.7); 1.580(0.7); 1.574(0.6); −0.014(0.9); −0.024(13.2)
Example 350: ¹H-NMR(299.9 MHz, dmso):
δ = 8.781(0.9); 8.752(0.9); 8.450(2.7); 8.419(2.8); 7.551(1.5); 7.544(1.7); 7.504(5.8); 7.475(2.9); 7.468(1.8); 7.460(1.5); 7.445(2.1); 7.440(2.5); 7.418(4.4); 7.390(2.8); 7.368(1.1); 7.339(1.0); 7.312(16.0);
7.278(0.8); 7.125(1.7); 7.099(2.5); 7.076(5.8); 7.050(7.3); 7.023(4.3); 4.797(0.9); 4.777(1.9); 4.753(1.9); 4.732(0.9); 4.584(0.6); 4.554(0.9); 4.526(0.6); 3.515(1.2); 3.493(2.4); 3.472(2.3); 3.454(2.4);
3.432(1.5); 3.392(1.6); 3.320(218.8); 3.222(0.5); 3.186(0.4); 2.727(0.4); 2.506(41.3); 2.500(52.4); 2.495(40.1); 2.271(0.6); 2.246(0.8); 2.219(1.4); 2.203(2.1); 2.193(2.0); 2.180(2.5); 2.165(1.8); 2.138(1.3);
2.121(1.0); 2.086(1.1); 2.046(1.8); 2.017(1.5); 1.978(0.8); 1.946(0.7); 1.884(2.6); 1.863(3.2); 1.841(2.7); 1.821(2.3); 1.798(2.0); 1.771(1.2); 1.751(0.6); 1.692(1.7); 1.681(1.9); 1.655(3.2); 1.638(3.5);
1.616(2.4); 1.590(1.1); 1.560(0.6); 1.530(0.5); 1.491(0.6); 1.458(0.4); 1.241(0.5); 0.000(17.3)
Example 351: ¹H-NMR(299.9 MHz, dmso):
δ = 8.505(0.5); 8.477(0.5); 8.176(1.7); 8.145(1.7); 7.548(1.2); 7.541(1.5); 7.532(3.2); 7.528(4.0); 7.505(1.6); 7.439(0.5); 7.425(1.3); 7.418(2.0); 7.398(1.9); 7.394(1.7); 7.388(2.0); 7.371(16.0); 7.368(12.0);
7.355(3.4); 7.349(2.8); 7.340(1.1); 7.333(1.1); 7.329(1.3); 7.323(1.2); 7.317(0.7); 7.307(2.0); 7.300(1.6); 7.282(2.2); 7.276(2.1); 7.260(1.9); 7.254(1.6); 7.237(0.7); 7.231(0.5); 6.884(2.4); 6.860(2.3);
6.855(2.1); 4.803(0.4); 4.791(0.5); 4.779(0.9); 4.770(1.0); 4.758(0.8); 4.748(1.0); 4.727(0.5); 4.715(0.4); 4.584(0.4); 4.553(0.5); 4.525(0.3); 3.539(0.8); 3.517(1.6); 3.495(1.2); 3.478(1.4); 3.455(0.8);
3.431(0.6); 3.403(0.6); 3.320(72.2); 2.506(13.0); 2.501(17.2); 2.495(12.6); 2.240(0.4); 2.207(0.8); 2.195(1.0); 2.182(1.3); 2.163(1.3); 2.139(1.1); 2.116(0.9); 2.104(0.9); 2.073(1.2); 2.045(0.8); 2.034(0.6);
2.006(0.4); 1.907(0.9); 1.877(1.3); 1.860(1.6); 1.837(1.3); 1.821(1.3); 1.798(1.2); 1.772(0.7); 1.748(0.6); 1.734(0.6); 1.723(1.0); 1.710(0.9); 1.696(1.1); 1.678(1.2); 1.666(1.3); 1.656(1.7); 1.632(1.1);
1.601(0.7); 1.565(0.4); 1.538(0.4); 0.010(0.5); 0.000(8.7)
Example 352: ¹H-NMR(299.9 MHz, dmso):
δ = 8.464(0.9); 8.437(0.9); 8.120(2.4); 8.090(2.4); 7.832(1.3); 7.806(1.5); 7.797(3.4); 7.794(3.8); 7.771(3.5); 7.768(3.8); 7.547(1.8); 7.539(3.6); 7.534(3.6); 7.530(4.9); 7.527(3.9); 7.510(2.5); 7.437(1.5);
7.430(1.5); 7.417(1.1); 7.409(1.3); 7.387(12.5); 7.384(16.0); 7.371(2.0); 7.368(1.7); 7.360(2.1); 7.357(2.3); 7.335(3.8); 7.332(4.3); 7.310(2.4); 7.307(2.6); 7.152(1.8); 7.147(1.7); 7.122(2.0); 7.112(2.5);
7.107(2.6); 7.097(1.1); 7.086(3.2); 7.081(3.6); 7.061(1.7); 7.055(1.8); 6.704(3.2); 6.699(3.6); 6.679(3.1); 6.674(3.2); 4.759(0.6); 4.737(1.4); 4.716(1.4); 4.695(0.7); 4.683(0.5); 4.554(0.6); 4.524(0.8);
4.497(0.5); 3.540(1.0); 3.517(2.0); 3.496(1.6); 3.479(1.8); 3.457(1.0); 3.437(0.8); 3.409(0.8); 3.375(0.6); 3.315(189.9); 2.726(0.5); 2.512(19.5); 2.506(43.2); 2.500(62.3); 2.494(50.3); 2.488(27.5);
2.270(0.6); 2.225(0.9); 2.200(1.3); 2.187(1.4); 2.176(1.8); 2.156(1.9); 2.131(1.4); 2.118(1.6); 2.080(1.5); 2.052(1.2); 2.013(0.6); 1.955(0.5); 1.916(1.0); 1.900(1.1); 1.878(1.6); 1.858(2.0); 1.836(1.6);
1.818(1.9); 1.792(2.1); 1.770(1.9); 1.753(1.7); 1.725(1.6); 1.714(1.2); 1.691(1.5); 1.664(1.2); 1.637(1.2); 1.618(0.8); 1.603(1.0); 1.550(0.3); 1.523(0.4); 1.488(0.5); 1.481(0.5); 1.447(0.4); 1.240(0.4);
0.010(1.5); −0.001(38.4)
Example 353: ¹H-NMR(299.9 MHz, dmso):
δ = 8.583(1.2); 8.554(1.2); 8.233(2.3); 8.203(2.3); 7.737(1.3); 7.708(2.5); 7.699(2.6); 7.675(4.1); 7.669(3.7); 7.656(1.5); 7.634(1.3); 7.613(3.1); 7.590(4.9); 7.583(4.7); 7.560(5.3); 7.552(7.5); 7.519(2.0);
7.490(3.2); 7.416(16.0); 7.412(15.3); 7.396(1.6); 7.389(1.4); 7.329(1.7); 7.305(1.5); 6.849(2.9); 6.827(2.6); 4.768(1.4); 4.744(1.5); 4.723(0.7); 4.711(0.5); 4.572(0.8); 4.543(1.1); 4.515(0.8); 3.542(1.0);
3.519(2.1); 3.498(1.7); 3.480(2.0); 3.459(1.5); 3.430(1.3); 3.401(1.4); 3.319(127.0); 2.727(0.4); 2.512(20.1); 2.507(39.9); 2.501(54.0); 2.495(41.5); 2.271(0.5); 2.227(0.9); 2.196(1.3); 2.182(1.8); 2.170(2.0);
2.161(2.2); 2.135(2.0); 2.095(2.1); 2.067(1.1); 2.054(0.9); 2.026(0.6); 1.946(0.4); 1.922(0.8); 1.892(1.8); 1.871(2.6); 1.844(2.1); 1.832(1.8); 1.815(1.7); 1.793(1.8); 1.766(1.4); 1.745(0.7); 1.706(1.2);
1.683(2.0); 1.672(1.9); 1.649(2.7); 1.643(2.7); 1.631(2.7); 1.608(1.5); 1.582(0.6); 1.559(0.4); 1.529(0.6); 1.496(0.7); 1.487(0.7); 1.468(0.3); 1.454(0.5); 0.000(30.1)
Example 354: ¹H-NMR(299.9 MHz, dmso):
δ = 7.772(1.1); 7.742(1.1); 7.497(1.9); 7.492(1.9); 7.348(0.7); 7.320(4.2); 7.314(3.3); 7.308(2.5); 7.286(0.4); 7.279(0.4); 4.706(0.5); 4.692(0.6); 4.677(0.6); 4.667(0.5); 3.539(0.5); 3.516(1.0); 3.493(0.7);
3.478(1.0); 3.455(0.5); 3.320(118.3); 2.569(1.8); 2.551(14.9); 2.512(13.1); 2.506(25.5); 2.500(33.4); 2.494(23.8); 2.488(11.0); 2.352(1.3); 2.180(0.4); 2.168(0.6); 2.156(0.6); 2.112(16.0); 2.057(0.6);
2.031(0.6); 1.953(0.3); 1.916(0.5); 1.892(0.6); 1.878(0.7); 1.857(0.9); 1.833(0.6); 1.818(0.6); 1.794(0.4); 1.737(0.6); 1.722(0.6); 1.709(0.5); 1.694(0.7); 1.678(0.5); 1.666(0.5); 1.654(0.4); 1.638(0.4);
1.614(0.5); 1.575(0.5); 0.010(0.8); −0.001(17.3); −0.011(0.7)
Example 355: ¹H-NMR(299.9 MHz, dmso):
δ = 8.209(4.5); 8.178(4.6); 8.133(0.4); 7.734(0.4); 7.688(4.1); 7.668(5.3); 7.658(6.0); 7.643(1.3); 7.597(7.4); 7.593(8.1); 7.571(13.2); 7.567(16.0); 7.554(13.9); 7.542(8.4); 7.517(1.6); 7.509(1.4); 7.466(0.5);
7.439(3.1); 7.433(3.5); 7.413(8.7); 7.407(8.6); 7.398(6.0); 7.395(5.7); 7.374(6.4); 7.371(6.6); 7.349(2.6); 7.345(2.4); 7.323(0.7); 7.306(1.3); 7.292(1.3); 7.282(1.4); 7.270(1.4); 7.246(0.7); 7.209(4.0);
7.202(4.1); 7.182(5.2); 7.179(5.0); 7.159(3.0); 7.152(2.8); 6.700(4.5); 6.690(4.8); 6.671(4.5); 4.843(1.0); 4.833(1.3); 4.811(2.9); 4.798(2.2); 4.788(2.9); 4.766(1.3); 4.755(1.0); 3.546(1.8); 3.524(3.7);
3.503(3.2); 3.484(3.6); 3.462(1.8); 3.439(0.4); 3.325(51.8); 2.507(24.4); 2.501(31.9); 2.495(23.6); 2.269(0.4); 2.230(1.1); 2.202(2.3); 2.185(2.5); 2.175(4.1); 2.164(3.8); 2.148(2.9); 2.132(4.3); 2.105(2.6);
2.093(1.9); 2.065(1.1); 2.043(0.4); 2.030(0.5); 2.016(0.4); 2.002(0.6); 1.974(0.4); 1.948(0.7); 1.923(1.7); 1.899(3.6); 1.888(3.4); 1.874(5.2); 1.855(3.4); 1.838(3.1); 1.812(1.5); 1.791(0.6); 1.763(0.4);
1.741(0.4); 1.706(1.7); 1.694(1.6); 1.682(3.1); 1.672(3.1); 1.656(4.5); 1.649(4.7); 1.642(4.4); 1.630(4.9); 1.607(2.2); 1.234(0.4); 0.000(22.8); −0.011(1.1)
Example 356: ¹H-NMR(299.9 MHz, dmso):
δ = 8.682(0.5); 8.653(0.4); 8.435(0.8); 8.428(0.9); 8.419(1.0); 8.412(1.0); 8.401(5.3); 8.394(5.6); 8.385(5.8); 8.378(5.5); 8.332(3.5); 8.302(3.4); 7.736(0.7); 7.730(0.7); 7.711(0.9); 7.705(0.8); 7.554(1.3);
7.546(1.6); 7.538(6.2); 7.533(6.8); 7.507(1.4); 7.466(1.0); 7.450(1.0); 7.441(1.2); 7.433(4.8); 7.425(1.8); 7.418(4.5); 7.408(8.2); 7.402(2.9); 7.393(8.7); 7.374(10.8); 7.367(16.0); 7.365(15.8); 7.354(8.5);
7.347(8.5); 7.336(2.1); 7.329(4.5); 7.322(3.9); 6.528(1.7); 4.795(0.8); 4.784(0.9); 4.771(1.7); 4.763(2.1); 4.751(1.7); 4.741(2.2); 4.720(1.0); 4.709(0.8); 4.546(0.4); 3.543(1.5); 3.521(3.1); 3.503(2.2);
3.482(2.8); 3.460(1.5); 3.423(0.6); 3.396(0.4); 3.389(0.4); 3.360(0.6); 3.319(169.8); 3.296(3.3); 3.244(0.4); 3.137(0.3); 2.513(10.2); 2.507(21.3); 2.501(29.2); 2.495(21.4); 2.489(10.3); 2.271(0.4);
2.249(0.9); 2.219(1.4); 2.204(1.9); 2.193(2.0); 2.179(2.3); 2.150(1.5); 2.126(1.0); 2.096(1.2); 2.056(2.3); 2.029(1.6); 2.017(1.4); 1.990(1.0); 1.959(0.5); 1.904(2.1); 1.895(2.2); 1.876(3.8); 1.854(2.6);
1.838(2.3); 1.810(1.4); 1.780(0.7); 1.748(1.0); 1.737(1.1); 1.725(1.9); 1.714(1.8); 1.700(2.5); 1.672(3.5); 1.658(2.8); 1.626(1.4); 1.596(0.7); 1.557(0.4); 1.513(0.4); 0.995(0.3); 0.971(0.5); 0.010(0.6); −0.001
(16.1); −0.011(0.7)
Example 357: ¹H-NMR(299.9 MHz, dmso):
δ = 8.478(1.8); 8.451(1.9); 8.179(2.1); 8.151(2.5); 8.094(7.8); 8.076(4.6); 8.062(7.9); 7.828(2.8); 7.802(2.9); 7.783(11.2); 7.757(13.3); 7.734(2.3); 7.708(1.5); 7.577(14.0); 7.551(15.9); 7.525(1.3);
7.493(2.0); 7.472(3.0); 7.446(0.7); 7.409(5.3); 7.402(6.5); 7.384(15.9); 7.377(16.0); 7.369(11.1); 7.345(11.3); 7.316(9.0); 7.291(12.3); 7.266(7.5); 7.186(5.9); 7.179(6.1); 7.158(9.8); 7.136(7.7); 7.130(6.5);
7.117(4.5); 7.093(6.9); 7.088(7.3); 7.068(10.0); 7.063(10.0); 7.042(5.0); 7.037(4.8); 6.640(6.2); 6.559(10.0); 6.554(10.3); 6.534(9.7); 6.529(9.3); 4.814(1.7); 4.802(2.1); 4.789(3.6); 4.780(4.6); 4.768(3.5);
4.757(4.6); 4.749(3.6); 4.735(2.1); 4.724(1.7); 4.589(1.3); 4.562(1.6); 4.532(1.0); 4.505(0.4); 3.542(3.0); 3.519(6.0); 3.499(5.2); 3.480(6.1); 3.456(3.7); 3.422(1.7); 3.391(0.9); 3.324(150.2); 2.728(0.8);
2.507(82.4); 2.501(107.4); 2.495(79.7); 2.278(1.7); 2.238(2.1); 2.205(3.1); 2.192(4.5); 2.180(4.7); 2.162(7.0); 2.134(4.8); 2.123(6.4); 2.093(3.9); 2.054(1.8); 1.958(1.5); 1.918(3.5); 1.903(3.6); 1.885(5.1);
1.863(6.4); 1.840(4.8); 1.822(4.7); 1.799(5.5); 1.777(4.7); 1.765(5.2); 1.749(4.6); 1.735(4.4); 1.721(3.7); 1.696(4.5); 1.665(3.2); 1.641(3.8); 1.628(2.8); 1.604(2.9); 1.578(1.3); 1.546(0.5); 1.506(0.5);
1.475(0.9); 1.443(1.0); 1.402(0.7); 1.336(0.4); 1.235(2.9); 0.858(0.4); 0.833(0.3); 0.195(0.4); 0.000(77.5); −0.199(0.4)
Example 358: ¹H-NMR(299.9 MHz, dmso):
δ = 8.760(2.0); 8.745(3.4); 8.731(6.0); 8.718(6.5); 8.380(4.5); 8.349(4.4); 7.813(1.2); 7.791(2.3); 7.757(2.2); 7.742(2.2); 7.722(4.8); 7.707(4.8); 7.696(5.3); 7.681(5.0); 7.564(9.0); 7.557(10.1); 7.522(2.2);
7.494(3.9); 7.432(3.8); 7.425(3.2); 7.407(11.4); 7.401(12.6); 7.392(16.0); 7.364(3.0); 7.338(6.1); 7.314(5.0); 4.797(1.1); 4.788(1.4); 4.767(2.8); 4.744(2.8); 4.722(1.3); 4.595(0.4); 4.566(0.9); 4.538(1.2);
4.509(0.9); 4.480(0.3); 3.610(0.4); 3.546(2.0); 3.525(3.8); 3.505(3.2); 3.486(4.1); 3.464(2.5); 3.446(1.6); 3.413(2.5); 3.319(575.2); 3.239(1.5); 3.185(0.7); 3.168(0.6); 3.134(0.6); 3.094(0.3); 2.727(1.6);
2.618(0.4); 2.512(79.4); 2.507(154.2); 2.501(206.1); 2.495(154.5); 2.387(0.4); 2.271(1.8); 2.210(2.5); 2.186(3.7); 2.171(3.3); 2.141(2.3); 2.103(1.8); 2.065(2.9); 2.036(1.9); 1.997(1.1); 1.942(1.0);
1.886(5.2); 1.865(3.9); 1.851(3.4); 1.826(2.8); 1.800(2.0); 1.777(1.7); 1.754(0.8); 1.732(0.9); 1.704(2.5); 1.679(4.4); 1.652(5.7); 1.634(3.7); 1.606(1.6); 1.575(0.7); 1.549(0.7); 1.507(0.9); 1.474(0.8);
1.430(0.5); 1.234(1.3); 1.145(0.3); 0.854(0.4); 0.195(0.5); 0.000(91.6); −0.199(0.5)
Example 359: ¹H-NMR(399.8 MHz, dmso):
δ = 7.849(4.8); 7.461(2.8); 7.456(2.9); 7.334(2.0); 7.313(4.0); 7.287(1.5); 7.253(2.0); 7.248(1.9); 7.232(1.3); 7.227(1.2); 4.662(0.7); 4.648(0.8); 3.711(16.0); 3.500(0.5); 3.484(1.0); 3.468(0.8); 3.455(1.1);
3.439(0.6); 3.391(0.4); 3.374(0.4); 3.315(29.8); 2.505(22.3); 2.500(28.5); 2.496(21.3); 2.220(0.9); 2.175(0.3); 2.149(0.6); 2.142(0.7); 2.132(0.8); 2.122(0.8); 2.100(0.7); 2.072(0.6); 2.049(0.9); 2.010(15.6);
1.988(0.6); 1.933(0.7); 1.926(0.7); 1.916(0.6); 1.904(0.7); 1.897(0.6); 1.875(0.4); 1.868(0.5); 1.850(0.9); 1.833(0.8); 1.820(0.7); 1.803(0.5); 1.712(0.3); 1.702(0.4); 1.693(0.6); 1.682(0.7); 1.672(0.7);
1.661(1.0); 1.643(1.2); 1.624(0.8); 1.599(0.6); 1.108(0.3); 1.090(0.7); −0.001(17.0)
Example 360: ¹H-NMR(400.0 MHz, DMSO):
δ = 7.866(2.5); 7.845(2.5); 7.675(0.6); 7.662(0.8); 7.650(0.5); 7.633(0.4); 7.603(6.2); 7.595(9.1); 7.583(16.0); 7.557(0.4); 7.469(7.4); 7.448(5.6); 7.165(0.7); 7.152(0.7); 7.100(8.5); 7.088(8.1); 4.658(0.6);
4.640(1.5); 4.623(1.8); 4.605(1.5); 4.588(0.6); 3.456(0.9); 3.435(2.2); 3.415(2.2); 3.395(0.9); 3.325(44.3); 2.891(0.3); 2.676(0.4); 2.671(0.5); 2.667(0.4); 2.511(31.4); 2.507(60.5); 2.502(77.5); 2.498(55.6);
2.493(26.9); 2.333(0.4); 2.329(0.5); 2.324(0.4); 2.152(0.7); 2.140(0.8); 2.134(0.8); 2.129(0.9); 2.119(1.6); 2.110(1.5); 2.101(1.5); 2.096(1.2); 2.091(1.3); 2.086(1.3); 2.079(2.3); 2.065(3.0); 2.058(4.0);
2.044(4.4); 2.037(2.6); 2.022(2.1); 1.981(0.5); 1.970(0.7); 1.955(1.2); 1.940(1.5); 1.922(1.2); 1.909(0.9); 1.895(0.5); 1.808(0.9); 1.794(1.0); 1.788(1.4); 1.775(1.9); 1.768(1.1); 1.761(0.9); 1.755(1.8);
1.742(1.1); 1.736(1.0); 1.725(1.1); 1.708(1.5); 1.687(1.7); 1.680(1.2); 1.667(1.0); 1.658(1.3); 1.646(0.4); 1.636(0.8); 0.146(0.5); 0.019(0.4); 0.008(4.4); 0.000(104.4); −0.009(4.2); −0.150(0.5)
Example 361: ¹H-NMR(399.8 MHz, dmso):
δ = 8.709(7.6); 8.700(7.4); 8.698(7.5); 8.338(5.7); 8.314(5.8); 7.670(6.5); 7.658(6.6); 7.651(7.2); 7.639(6.8); 7.424(8.1); 7.421(8.6); 7.405(15.2); 7.402(16.0); 7.387(9.4); 7.383(9.6); 7.346(4.5); 7.343(4.9);
7.328(9.3); 7.324(8.4); 7.309(5.3); 7.305(4.5); 7.282(7.0); 7.277(6.8); 7.263(8.0); 7.259(7.7); 7.244(3.5); 7.240(3.1); 7.120(7.6); 7.117(7.9); 7.100(7.2); 7.098(7.2); 4.824(1.3); 4.816(1.6); 4.806(2.8);
4.800(3.7); 4.790(2.6); 4.782(3.7); 4.777(2.7); 4.766(1.6); 4.758(1.3); 3.581(2.5); 3.564(4.9); 3.549(4.0); 3.534(4.9); 3.518(2.5); 3.316(118.2); 2.509(16.3); 2.505(34.4); 2.500(47.3); 2.496(34.2);
2.491(15.8); 2.234(1.1); 2.213(2.1); 2.203(2.7); 2.194(3.1); 2.184(3.3); 2.175(2.3); 2.162(2.0); 2.138(2.2); 2.115(1.8); 2.107(3.8); 2.085(2.5); 2.077(2.2); 2.055(1.2); 1.934(1.0); 1.926(0.8); 1.900(4.4);
1.884(6.8); 1.871(5.0); 1.856(4.0); 1.839(1.7); 1.711(0.9); 1.690(2.6); 1.680(3.3); 1.672(5.3); 1.659(5.3); 1.652(6.8); 1.639(4.2); 1.632(3.7); 1.622(1.5); 1.613(1.5); 1.592(0.4); 1.262(0.5); 1.175(0.4);
1.158(0.4); 1.087(0.4); 1.045(0.7); 1.030(0.7); 0.859(0.7); 0.841(0.4); 0.008(1.7); 0.000(42.6); −0.009(1.4)
Example 362: ¹H-NMR(399.8 MHz, dmso):
δ = 8.719(6.7); 8.717(7.0); 8.708(6.9); 8.705(6.9); 8.598(5.4); 8.578(5.4); 7.685(6.3); 7.673(6.3); 7.666(6.8); 7.654(6.6); 7.352(1.7); 7.336(3.6); 7.331(3.3); 7.319(2.5); 7.315(7.2); 7.310(2.8); 7.299(3.4);
7.294(4.2); 7.278(2.0); 7.124(7.1); 7.121(7.4); 7.104(6.8); 7.102(6.8); 7.058(9.9); 7.036(16.0); 7.015(8.2); 4.616(1.3); 4.597(4.1); 4.578(6.3); 4.559(4.3); 4.540(1.4); 3.669(2.6); 3.648(5.4); 3.643(3.4);
3.628(3.4); 3.622(5.4); 3.602(2.6); 3.425(0.3); 3.414(0.4); 3.363(0.5); 3.315(500.0); 3.264(0.4); 2.674(0.5); 2.669(0.7); 2.665(0.4); 2.509(34.4); 2.505(73.8); 2.500(101.3); 2.496(72.7); 2.491(32.8);
2.332(0.5); 2.327(0.6); 2.323(0.5); 2.182(0.6); 2.156(1.8); 2.152(1.7); 2.132(3.4); 2.126(3.6); 2.106(5.1); 2.095(3.3); 2.082(3.4); 2.076(3.2); 2.063(1.4); 2.056(1.4); 2.028(1.6); 2.009(3.2); 1.994(2.4);
1.980(2.1); 1.959(1.1); 1.939(0.9); 1.933(1.2); 1.921(1.7); 1.914(2.3); 1.908(2.3); 1.902(2.3); 1.890(2.4); 1.884(2.7); 1.877(1.7); 1.865(1.4); 1.785(1.4); 1.767(2.3); 1.761(2.1); 1.743(3.0); 1.736(2.1);
1.729(2.2); 1.711(2.5); 1.693(1.3); 1.641(0.8); 1.620(1.6); 1.594(2.6); 1.569(2.1); 1.545(1.1); 1.521(0.4); 1.387(2.3); 1.280(2.7); 0.008(3.0); −0.001(81.7); −0.009(2.6); −0.150(0.3)
Example 363: ¹H-NMR(601.6 MHz, DMSO):
δ = 19.969(0.8); 8.321(0.8); 8.139(6.1); 8.124(6.2); 7.780(9.4); 7.768(9.7); 7.688(0.8); 7.670(1.0); 7.652(13.6); 7.639(16.0); 7.563(0.8); 7.521(0.6); 7.505(0.8); 7.480(14.4); 7.466(12.8); 7.400(0.6);
7.263(4.3); 7.262(4.5); 7.251(9.3); 7.249(9.6); 7.238(5.3); 7.237(5.5); 7.144(0.6); 7.080(4.7); 7.077(4.9); 7.067(7.8); 7.064(7.8); 7.054(4.3); 7.052(4.2); 6.505(7.7); 6.502(8.1); 6.492(7.9); 6.489(7.7);
4.680(1.3); 4.669(2.9); 4.657(3.6); 4.645(3.1); 4.633(1.3); 3.436(1.8); 3.422(4.5); 3.410(4.3); 3.395(2.0); 3.341(74.9); 3.339(99.2); 2.614(1.3); 2.524(2.1); 2.520(2.5); 2.517(2.6); 2.508(80.8); 2.505(174.2);
2.503(241.4); 2.500(177.4); 2.497(83.8); 2.387(1.3); 2.125(1.4); 2.117(1.5); 2.109(1.9); 2.102(2.9); 2.096(2.6); 2.090(2.9); 2.080(2.1); 2.075(1.9); 2.068(1.7); 2.054(2.7); 2.044(5.8); 2.039(7.5); 2.029(10.1);
2.024(5.6); 2.015(5.3); 1.943(0.9); 1.934(1.6); 1.922(2.3); 1.912(2.6); 1.900(2.3); 1.889(1.6); 1.779(1.6); 1.766(2.5); 1.757(3.8); 1.744(3.7); 1.735(2.5); 1.730(1.8); 1.722(1.5); 1.693(1.1); 1.679(2.7);
1.664(3.2); 1.658(2.4); 1.651(2.3); 1.644(2.6); 1.630(1.7); 1.616(0.7); 1.235(1.0); 0.097(1.2); 0.005(9.7); 0.000(275.5); −0.006(9.6); −0.100(1.2)
Example 364: ¹H-NMR(399.9 MHz, cdcl3):
δ = 7.653(1.5); 7.636(1.9); 7.630(2.0); 7.625(1.2); 7.614(4.5); 7.605(5.1); 7.601(5.2); 7.592(5.6); 7.582(1.2); 7.526(0.8); 7.521(1.0); 7.511(2.0); 7.507(2.0); 7.503(2.0); 7.496(4.1); 7.489(2.0); 7.483(1.8);
7.461(2.4); 7.450(14.0); 7.442(10.3); 7.436(10.3); 7.428(12.6); 7.417(1.6); 7.378(0.3); 7.346(0.5); 7.300(16.0); 7.279(24.0); 7.272(6.3); 7.262(101.2); 7.227(0.7); 7.206(21.7); 7.196(7.2); 7.189(7.3);
7.185(14.0); 7.178(3.7); 7.175(4.2); 7.052(1.4); 7.046(1.5); 7.031(1.4); 6.998(0.6); 6.922(0.9); 6.912(4.7); 6.904(5.1); 6.899(4.8); 6.890(4.2); 6.880(0.6); 5.731(0.8); 5.708(0.8); 5.397(3.0); 5.375(3.0);
5.302(2.0); 4.683(0.8); 4.675(1.0); 4.663(0.9); 4.653(0.8); 4.320(1.2); 4.310(1.2); 4.291(2.9); 4.282(2.5); 4.269(2.5); 4.259(2.7); 4.241(1.2); 4.231(1.1); 3.360(0.9); 3.225(0.5); 3.032(0.6); 3.023(1.1);
3.014(0.7); 3.002(0.7); 2.993(1.1); 2.983(0.6); 2.500(2.2); 2.492(2.5); 2.471(4.2); 2.463(4.2); 2.442(2.3); 2.433(2.2); 2.348(2.6); 2.342(2.7); 2.330(1.6); 2.317(2.7); 2.311(2.7); 2.147(0.8); 2.117(0.9);
1.958(2.9); 1.952(2.9); 1.926(4.3); 1.918(4.4); 1.909(3.9); 1.902(3.8); 1.868(3.0); 1.862(3.9); 1.855(4.3); 1.815(3.1); 1.786(0.7); 1.772(0.8); 1.763(1.2); 1.754(0.9); 1.732(1.7); 1.698(1.0); 1.643(0.9);
1.611(2.4); 1.575(12.6); 1.560(7.9); 1.551(7.0); 1.543(5.0); 1.536(3.9); 1.527(4.8); 1.519(5.5); 1.512(3.9); 1.503(2.3); 1.495(2.7); 1.487(2.8); 1.480(2.1); 1.470(1.1); 1.464(1.2); 1.441(1.2); 1.413(2.4);
1.405(1.6); 1.381(4.9); 1.348(5.1); 1.323(3.1); 1.315(3.4); 1.291(1.2); 1.282(1.1); 1.254(0.5); 1.111(0.5); 0.146(0.4); 0.008(5.0); 0.000(94.5); −0.008(4.8); −0.149(0.4)
Example 365: ¹H-NMR(400.0 MHz, DMSO):
δ = 8.432(2.8); 8.408(3.0); 8.315(1.3); 8.304(2.5); 8.281(2.6); 7.700(3.5); 7.673(9.2); 7.652(16.0); 7.630(9.1); 7.594(3.2); 7.574(4.2); 7.552(6.5); 7.537(3.4); 7.520(8.8); 7.498(11.4); 7.476(6.1); 6.977(3.9);
6.959(3.9); 6.783(2.8); 6.767(2.7); 4.494(1.9); 4.485(1.8); 4.478(2.0); 4.470(1.9); 4.128(0.4); 4.119(0.6); 4.100(1.4); 4.092(1.3); 4.078(1.3); 4.071(1.3); 4.050(0.7); 3.902(2.5); 3.401(0.3); 3.382(0.6);
3.324(376.0); 3.258(0.6); 3.239(0.4); 3.221(0.4); 3.067(2.0); 3.032(2.1); 2.737(1.0); 2.709(1.9); 2.701(1.8); 2.671(3.6); 2.541(3.0); 2.506(350.3); 2.502(435.7); 2.497(327.9); 2.409(0.7); 2.372(0.4);
2.359(0.4); 2.333(2.1); 2.329(2.6); 2.211(0.7); 2.178(1.6); 2.155(1.7); 2.147(1.6); 2.121(0.9); 1.970(1.5); 1.948(1.6); 1.844(1.5); 1.814(5.0); 1.780(6.2); 1.739(2.8); 1.715(1.7); 1.706(1.6); 1.682(0.8);
1.633(2.3); 1.607(3.1); 1.570(2.3); 1.538(4.8); 1.465(1.7); 1.435(1.5); 1.416(2.0); 1.384(2.8); 1.358(2.3); 1.336(2.0); 1.305(1.4); 1.274(0.6); 1.252(0.5); 1.236(0.5); 0.000(9.1)
Example 366: ¹H-NMR(399.9 MHz, cdcl3):
δ = 7.263(37.7); 7.252(1.5); 7.247(1.9); 7.242(4.9); 7.168(4.8); 7.147(3.0); 5.299(0.7); 5.279(0.7); 4.148(0.7); 4.139(0.6); 4.128(0.5); 4.118(0.6); 2.635(2.0); 2.597(16.0); 2.511(0.5); 2.502(0.6); 2.482(1.0);
2.474(2.4); 2.453(0.5); 2.444(0.5); 2.350(15.9); 2.331(0.7); 2.325(0.7); 2.312(0.4); 2.299(0.7); 2.294(0.7); 1.955(0.7); 1.922(0.8); 1.886(0.6); 1.879(0.6); 1.848(1.3); 1.813(0.7); 1.568(5.2); 1.536(0.8);
1.520(1.2); 1.512(1.0); 1.504(0.6); 1.495(0.5); 1.487(0.6); 1.478(0.5); 1.414(0.5); 1.405(0.3); 1.390(0.5); 1.381(0.8); 1.372(0.5); 1.366(0.5); 1.357(0.7); 1.349(0.7); 1.334(0.8); 1.326(0.7); 1.317(0.3);
1.305(0.6); 1.297(0.6); 0.007(2.0); −0.001(36.1); −0.008(2.0)
Example 367: ¹H-NMR(399.9 MHz, cdcl3):
δ = 7.555(1.1); 7.457(4.0); 7.263(40.7); 7.250(3.7); 7.245(1.8); 7.235(3.4); 7.229(5.6); 7.184(2.0); 7.171(5.4); 7.150(3.1); 5.232(0.8); 5.212(0.8); 4.174(0.7); 4.165(0.6); 4.154(0.6); 4.145(0.7); 3.789(6.0);
3.755(16.0); 3.715(0.5); 3.012(0.3); 2.982(0.3); 2.510(0.5); 2.501(0.6); 2.481(1.0); 2.472(1.0); 2.452(0.6); 2.444(0.5); 2.385(0.5); 2.347(0.7); 2.340(0.8); 2.322(5.5); 2.173(15.8); 2.091(0.3); 1.947(1.1);
1.914(0.9); 1.870(0.7); 1.836(1.4); 1.807(0.8); 1.723(0.5); 1.715(0.5); 1.699(0.5); 1.691(0.5); 1.679(0.6); 1.653(0.3); 1.575(7.0); 1.551(1.1); 1.540(1.1); 1.530(1.1); 1.518(1.1); 1.508(1.2); 1.500(0.9);
1.484(0.7); 1.476(0.8); 1.467(0.6); 1.436(0.3); 1.424(0.3); 1.414(0.5); 1.406(0.5); 1.391(0.5); 1.382(0.8); 1.373(0.5); 1.358(0.5); 1.349(0.7); 1.336(0.5); 1.327(0.5); 1.304(0.8); 1.295(0.7); 1.275(0.6);
1.267(0.7); −0.001(40.7); −0.009(2.0)
Example 368: ¹H-NMR(399.9 MHz, cdcl3):
δ = 8.722(0.9); 8.710(0.9); 8.679(4.3); 8.670(4.3); 8.667(4.2); 7.522(0.7); 7.496(0.6); 7.484(0.7); 7.477(1.0); 7.465(1.0); 7.435(3.5); 7.423(3.6); 7.415(4.7); 7.404(4.6); 7.391(0.8); 7.305(12.3); 7.300(5.3);
7.288(6.5); 7.284(18.9); 7.277(5.1); 7.273(7.4); 7.262(130.2); 7.255(7.1); 7.225(0.5); 7.221(0.5); 7.212(1.0); 7.197(16.0); 7.182(6.6); 7.176(11.7); 7.162(2.6); 6.998(0.8); 5.740(0.4); 5.720(0.5); 5.406(2.3);
5.385(2.4); 5.303(0.8); 4.688(0.5); 4.679(0.5); 4.666(0.5); 4.657(0.4); 4.320(0.8); 4.310(0.9); 4.291(2.2); 4.282(1.9); 4.269(1.9); 4.260(2.1); 4.241(0.9); 4.231(0.8); 3.357(1.2); 3.322(0.4); 3.039(0.6);
3.017(0.4); 3.008(0.6); 2.503(1.6); 2.494(1.7); 2.474(3.1); 2.465(3.0); 2.445(1.7); 2.436(1.6); 2.350(1.9); 2.344(2.0); 2.331(1.2); 2.319(2.0); 2.312(2.0); 2.153(0.5); 2.123(0.6); 1.968(2.2); 1.961(2.2);
1.928(3.9); 1.921(3.6); 1.888(2.2); 1.881(2.3); 1.874(2.1); 1.867(2.0); 1.860(2.0); 1.823(2.4); 1.798(0.5); 1.788(0.8); 1.753(1.0); 1.733(0.6); 1.721(0.6); 1.638(0.4); 1.608(1.3); 1.595(1.9); 1.586(2.4);
1.562(31.3); 1.541(3.2); 1.532(5.2); 1.524(3.8); 1.508(1.5); 1.500(2.6); 1.492(1.9); 1.470(0.6); 1.439(0.7); 1.427(1.1); 1.419(1.9); 1.411(1.7); 1.405(1.8); 1.396(2.8); 1.387(3.2); 1.374(3.1); 1.364(3.4);
1.355(2.5); 1.345(2.9); 1.336(2.3); 1.322(0.9); 1.312(1.2); 1.303(0.8); 1.256(0.4); 0.146(0.6); 0.037(0.4); 0.032(0.5); 0.007(7.4); 0.006(7.0); −0.001(132.8); −0.009(5.8); −0.016(0.5); −0.150(0.5)
Example 369: ¹H-NMR(399.9 MHz, cdcl3):
δ = 7.521(0.6); 7.356(0.5); 7.340(1.0); 7.335(0.9); 7.324(0.8); 7.319(1.6); 7.314(0.7); 7.303(2.1); 7.298(1.4); 7.287(3.1); 7.281(4.9); 7.276(12.0); 7.271(7.9); 7.262(98.2); 7.255(20.8); 7.245(3.7); 7.229(1.4);
7.203(6.1); 7.193(16.0); 7.188(6.0); 7.182(4.2); 7.176(4.5); 7.172(9.4); 6.998(0.6); 6.938(0.3); 6.915(0.8); 6.909(2.8); 6.889(4.1); 6.868(2.4); 6.856(1.2); 6.852(1.6); 6.846(7.2); 6.826(10.2); 6.806(6.1);
6.799(1.3); 5.924(0.6); 5.904(0.6); 5.566(1.8); 5.546(1.9); 4.683(0.8); 4.674(0.9); 4.663(0.8); 4.653(0.7); 4.325(0.7); 4.315(0.8); 4.296(2.0); 4.287(1.7); 4.274(1.7); 4.265(1.9); 4.246(0.8); 4.236(0.7);
3.380(0.8); 3.343(0.3); 3.020(0.5); 3.012(0.9); 2.990(0.6); 2.981(1.0); 2.973(0.7); 2.522(1.5); 2.513(1.6); 2.493(2.9); 2.484(2.8); 2.464(1.6); 2.455(1.5); 2.361(1.8); 2.354(1.9); 2.341(1.2); 2.329(1.9);
2.322(1.9); 2.161(0.7); 2.128(0.9); 1.952(2.6); 1.944(2.2); 1.919(3.8); 1.897(1.9); 1.857(2.9); 1.852(3.1); 1.812(2.1); 1.781(0.5); 1.758(1.0); 1.724(1.4); 1.690(1.1); 1.678(1.2); 1.653(0.8); 1.623(0.5);
1.564(16.4); 1.548(4.6); 1.541(3.1); 1.530(2.1); 1.522(3.3); 1.515(3.8); 1.498(1.4); 1.483(2.6); 1.456(1.3); 1.417(2.5); 1.409(1.9); 1.385(4.2); 1.378(3.1); 1.352(3.3); 1.326(1.0); 1.318(1.1); 0.145(0.4);
0.022(0.5); 0.007(5.2); −0.001(96.6); −0.009(4.0); −0.150(0.4)
Example 370: ¹H-NMR(399.9 MHz, cdcl3):
δ = 8.431(2.5); 8.426(2.7); 8.419(2.7); 8.414(2.7); 8.382(7.2); 8.377(7.7); 8.370(7.7); 8.365(7.5); 7.952(2.7); 7.947(2.7); 7.933(2.8); 7.928(2.7); 7.737(7.6); 7.732(7.8); 7.718(8.3); 7.713(8.1); 7.522(1.1);
7.346(0.4); 7.333(0.4); 7.312(3.1); 7.300(3.7); 7.293(3.8); 7.281(6.9); 7.275(17.2); 7.263(192.4); 7.254(37.4); 7.243(13.7); 7.242(13.8); 7.234(9.3); 7.222(8.4); 7.203(27.1); 7.197(16.0); 7.181(15.6);
7.176(6.8); 6.999(1.2); 6.677(1.0); 6.657(1.1); 6.027(3.3); 6.007(3.4); 4.679(1.2); 4.670(1.3); 4.658(1.3); 4.649(1.1); 4.323(1.3); 4.313(1.4); 4.295(3.4); 4.285(2.9); 4.273(3.0); 4.264(3.2); 4.245(1.4);
4.235(1.3); 3.489(0.3); 3.383(0.7); 3.359(0.7); 3.043(0.9); 3.034(1.5); 3.026(1.0); 3.002(1.5); 2.551(2.5); 2.542(2.7); 2.522(4.7); 2.513(4.7); 2.493(2.6); 2.484(2.5); 2.355(3.3); 2.331(3.2); 2.323(3.3);
2.176(1.2); 2.148(1.4); 2.074(0.4); 2.059(0.4); 1.975(5.2); 1.941(5.1); 1.914(3.0); 1.909(3.0); 1.875(4.9); 1.870(5.3); 1.828(3.7); 1.784(1.7); 1.775(1.3); 1.755(2.6); 1.726(2.2); 1.702(1.4); 1.663(0.8);
1.603(3.0); 1.596(2.9); 1.563(89.3); 1.537(7.2); 1.501(4.5); 1.481(1.9); 1.430(3.4); 1.423(2.9); 1.399(6.8); 1.392(5.0); 1.368(5.5); 1.361(4.2); 1.337(1.7); 1.329(1.3); 1.253(0.5); 1.229(0.3); 1.211(0.5);
0.883(0.5); 0.146(1.0); 0.121(0.3); 0.063(0.5); 0.000(186.9); −0.008(7.9); −0.150(0.7)
Example 371: ¹H-NMR(399.9 MHz, cdcl3):
δ = 7.521(0.8); 7.451(2.3); 7.448(2.4); 7.433(2.8); 7.429(2.9); 7.391(0.4); 7.359(1.0); 7.355(1.5); 7.336(5.8); 7.320(3.6); 7.316(3.1); 7.300(2.3); 7.296(2.3); 7.274(29.5); 7.262(156.7); 7.254(32.9);
7.247(18.8); 7.244(16.6); 7.241(16.8); 7.222(8.8); 7.213(11.8); 7.209(30.1); 7.203(21.4); 7.187(16.0); 7.182(8.5); 7.065(0.4); 6.999(0.9); 6.311(1.1); 6.290(1.2); 5.760(3.3); 5.739(3.3); 4.687(1.3);
4.679(1.5); 4.667(1.4); 4.658(1.2); 4.331(1.3); 4.321(1.4); 4.302(3.4); 4.293(3.0); 4.281(2.9); 4.271(3.3); 4.265(1.8); 4.253(1.5); 4.243(1.3); 3.379(0.9); 3.020(1.6); 3.012(1.0); 2.996(1.1); 2.988(1.7);
2.530(2.5); 2.521(2.8); 2.501(4.8); 2.492(4.7); 2.472(2.7); 2.463(2.5); 2.367(3.0); 2.362(3.2); 2.348(2.0); 2.336(3.3); 2.329(3.2); 2.186(1.3); 2.154(1.5); 2.013(0.3); 1.960(5.2); 1.929(6.5); 1.898(3.1);
1.858(5.9); 1.853(5.6); 1.817(3.6); 1.790(0.9); 1.774(1.3); 1.765(1.7); 1.756(1.3); 1.730(2.6); 1.706(2.1); 1.694(2.2); 1.670(1.5); 1.632(1.0); 1.599(2.9); 1.590(4.8); 1.568(32.7); 1.542(4.9); 1.534(5.6);
1.526(6.4); 1.519(5.1); 1.510(3.3); 1.501(3.3); 1.494(4.2); 1.447(1.1); 1.421(2.3); 1.410(2.7); 1.397(3.3); 1.388(4.5); 1.379(5.3); 1.368(4.1); 1.356(3.7); 1.348(4.6); 1.340(3.3); 1.324(1.2); 1.317(1.7);
1.308(1.3); 1.253(0.8); 1.211(0.3); 0.146(0.7); 0.069(0.4); 0.055(0.4); 0.007(9.7); 0.000(136.4); −0.150(0.6)
Example 372: ¹H-NMR(399.9 MHz, cdcl3):
δ = 7.804(2.1); 7.786(2.4); 7.784(2.4); 7.763(9.3); 7.761(9.4); 7.743(9.7); 7.741(9.6); 7.521(1.3); 7.327(1.6); 7.324(1.7); 7.308(3.8); 7.305(4.1); 7.291(19.1); 7.286(11.2); 7.274(21.8); 7.270(43.2);
7.262(246.8); 7.245(12.8); 7.242(12.2); 7.225(13.1); 7.220(29.6); 7.209(8.8); 7.203(9.5); 7.199(16.0); 7.188(4.4); 7.073(1.3); 7.069(1.5); 7.054(2.0); 7.050(2.1); 7.034(1.6); 7.025(5.6); 7.021(6.0);
7.005(9.3); 7.002(10.4); 6.986(5.4); 6.983(5.7); 6.869(9.1); 6.865(8.7); 6.850(8.4); 6.846(7.8); 5.735(0.8); 5.715(0.8); 5.395(3.9); 5.373(3.9); 5.303(0.4); 4.670(0.9); 4.661(1.0); 4.649(1.0); 4.639(0.8);
4.318(1.5); 4.308(1.6); 4.289(3.7); 4.280(3.4); 4.267(3.3); 4.257(3.5); 4.239(1.6); 4.229(1.4); 4.131(0.9); 4.113(0.9); 4.096(0.4); 3.034(1.0); 3.025(0.7); 3.003(1.0); 2.513(2.6); 2.505(2.9); 2.484(5.3);
2.476(5.1); 2.455(3.0); 2.446(2.7); 2.405(3.3); 2.399(3.5); 2.385(2.3); 2.373(3.6); 2.367(3.5); 2.232(0.8); 2.208(0.9); 2.074(0.4); 2.048(4.2); 1.959(4.2); 1.952(4.3); 1.944(3.4); 1.919(6.4); 1.875(3.9);
1.868(4.5); 1.861(4.6); 1.854(4.1); 1.817(3.7); 1.762(1.2); 1.753(1.2); 1.736(1.4); 1.660(1.1); 1.635(1.3); 1.600(2.7); 1.590(4.1); 1.559(119.7); 1.535(7.2); 1.527(7.8); 1.519(5.9); 1.502(4.4); 1.494(4.3);
1.487(2.7); 1.430(2.5); 1.423(3.3); 1.418(3.1); 1.394(6.1); 1.386(5.6); 1.377(3.5); 1.371(4.2); 1.362(5.5); 1.354(4.1); 1.345(2.3); 1.339(1.9); 1.330(1.9); 1.321(1.2); 1.312(0.7); 1.279(1.3); 1.261(2.6);
1.243(1.2); 0.146(1.1); 0.070(0.6); 0.050(0.9); 0.000(231.5); −0.149(0.9)
Example 373: ¹H-NMR(400.0 MHz, CD3CN):
δ = 7.751(12.9); 7.750(13.9); 7.732(14.8); 7.730(16.0); 7.661(6.4); 7.641(7.3); 7.614(3.7); 7.595(6.7); 7.576(3.2); 7.418(0.4); 7.399(0.3); 7.365(4.1); 7.345(6.6); 7.326(3.1); 7.271(4.5); 7.268(4.3);
7.252(10.0); 7.249(9.2); 7.233(5.7); 7.231(5.2); 7.043(5.0); 7.038(5.1); 7.023(7.3); 7.019(7.3); 7.004(4.1); 7.000(4.0); 6.689(8.1); 6.685(7.8); 6.670(7.6); 6.666(7.2); 6.340(1.9); 6.318(1.9); 4.467(1.0);
4.458(0.9); 4.440(2.5); 4.431(2.4); 4.414(2.5); 4.405(2.5); 4.387(1.0); 4.378(0.9); 2.981(1.7); 2.977(1.7); 2.952(3.4); 2.924(1.7); 2.920(1.7); 2.151(48.2); 2.124(1.8); 2.113(3.1); 2.106(3.5); 1.971(1.8);
1.964(5.0); 1.957(8.8); 1.952(40.7); 1.945(71.9); 1.939(93.8); 1.933(63.9); 1.927(33.3); 1.918(3.5); 1.910(3.8); 1.899(3.5); 1.884(4.7); 1.877(4.6); 1.868(4.2); 1.861(3.3); 1.855(2.2); 1.831(0.8); 1.823(2.0);
1.815(2.8); 1.812(2.6); 1.799(1.4); 1.780(3.3); 1.774(2.6); 1.762(0.9); 1.755(0.4); 1.647(0.8); 1.638(1.3); 1.626(1.5); 1.614(2.8); 1.605(3.7); 1.596(3.8); 1.582(4.1); 1.573(4.5); 1.564(4.7); 1.554(3.3);
1.540(2.4); 1.532(3.5); 1.528(3.5); 1.523(1.7); 1.506(3.5); 1.499(3.4); 1.477(2.7); 1.468(3.0); 1.444(1.3); 1.436(1.8); 1.429(1.2); 1.420(2.2); 1.411(1.3); 1.397(2.4); 1.388(4.5); 1.379(2.5); 1.365(1.9);
1.356(3.5); 1.347(1.8); 1.333(0.6); 1.323(1.0); 1.314(0.6); 1.221(0.4); 1.203(0.7); 1.185(0.4); 0.008(2.2); 0.000(55.1); −0.009(1.6)
Example 374: ¹H-NMR(400.0 MHz, CD3CN):
δ = 7.729(6.3); 7.709(8.4); 7.685(6.8); 7.665(7.7); 7.636(0.7); 7.626(4.8); 7.617(5.8); 7.611(6.8); 7.609(6.5); 7.606(5.5); 7.603(6.9); 7.593(7.4); 7.573(3.3); 7.503(1.0); 7.499(1.6); 7.498(1.5); 7.484(7.1);
7.480(11.8); 7.471(16.0); 7.462(9.9); 7.458(7.4); 7.444(1.5); 7.439(0.9); 7.393(4.4); 7.374(6.9); 7.355(3.1); 6.732(5.5); 6.726(3.3); 6.724(2.9); 6.718(5.5); 6.709(4.8); 6.699(0.5); 6.394(2.0); 6.373(2.0);
4.476(1.0); 4.466(1.0); 4.448(2.6); 4.439(2.6); 4.422(2.6); 4.413(2.6); 4.395(1.0); 4.386(1.0); 4.067(0.4); 4.049(0.4); 2.987(1.8); 2.983(1.8); 2.958(3.5); 2.930(1.8); 2.926(1.7); 2.132(12.6); 2.112(0.4);
2.106(0.5); 2.086(2.8); 2.080(2.8); 2.067(1.7); 2.056(3.0); 2.049(3.1); 1.971(2.2); 1.963(4.1); 1.957(7.3); 1.951(33.9); 1.945(60.1); 1.939(78.8); 1.932(54.1); 1.926(28.8); 1.916(3.0); 1.889(5.7); 1.867(2.2);
1.859(3.4); 1.853(3.3); 1.846(2.2); 1.830(0.9); 1.822(2.2); 1.818(2.4); 1.815(2.8); 1.811(2.8); 1.799(1.4); 1.790(2.4); 1.779(3.3); 1.773(2.6); 1.761(0.9); 1.755(0.3); 1.640(0.9); 1.631(1.5); 1.626(1.7);
1.617(1.5); 1.607(2.5); 1.598(5.2); 1.594(4.4); 1.586(3.4); 1.574(3.1); 1.566(6.6); 1.557(4.5); 1.542(1.5); 1.533(2.9); 1.512(2.4); 1.504(2.6); 1.482(3.5); 1.475(3.4); 1.473(3.3); 1.453(2.9); 1.444(3.4);
1.437(3.9); 1.428(1.5); 1.420(3.5); 1.411(2.4); 1.396(2.6); 1.388(5.0); 1.379(2.5); 1.364(2.1); 1.355(3.9); 1.346(1.9); 1.332(0.6); 1.323(1.1); 1.314(0.6); 1.221(0.6); 1.203(1.0); 1.185(0.5); 0.008(1.9);
0.000(51.0); −0.009(1.5)
Example 375: ¹H-NMR(400.0 MHz, DMSO):
δ = 8.687(8.6); 8.677(8.5); 8.675(8.5); 8.345(6.5); 8.322(6.6); 7.729(7.1); 7.709(9.9); 7.666(9.7); 7.662(8.6); 7.649(16.0); 7.630(8.3); 7.605(8.6); 7.586(4.2); 7.415(5.3); 7.396(8.7); 7.377(3.9); 7.173(8.9);
7.156(8.0); 7.154(8.1); 4.453(0.9); 4.443(1.0); 4.426(3.0); 4.401(3.3); 4.375(1.4); 4.056(0.7); 4.038(2.0); 4.020(2.0); 4.003(0.7); 3.026(0.5); 3.019(0.9); 3.010(0.9); 3.003(0.5); 2.926(2.3); 2.899(4.5);
2.873(2.3); 2.675(0.5); 2.671(0.7); 2.666(0.5); 2.541(0.3); 2.506(81.2); 2.502(105.8); 2.497(78.0); 2.333(0.5); 2.328(0.7); 2.324(0.5); 2.037(0.6); 1.996(1.0); 1.989(9.0); 1.944(4.3); 1.920(3.5); 1.824(5.7);
1.815(5.6); 1.802(6.2); 1.759(3.7); 1.731(4.4); 1.725(4.4); 1.569(1.0); 1.546(2.9); 1.538(3.0); 1.512(7.4); 1.483(10.7); 1.460(3.8); 1.454(4.0); 1.427(0.9); 1.421(0.9); 1.398(6.1); 1.320(1.6); 1.313(1.6);
1.289(3.3); 1.280(2.8); 1.275(3.6); 1.259(5.9); 1.244(4.1); 1.226(1.4); 1.193(2.5); 1.175(4.8); 1.157(2.4); 0.008(1.0); 0.000(24.8); −0.008(1.1)
Example 376: ¹H-NMR(299.9 MHz, dmso):
δ = 7.828(1.1); 7.797(1.0); 7.391(1.3); 7.386(1.5); 7.367(2.0); 7.361(2.1); 7.345(0.5); 7.308(0.7); 7.301(0.9); 7.283(1.9); 7.276(2.4); 7.266(1.3); 7.247(1.9); 7.242(1.8); 7.219(2.1); 7.211(1.7); 7.194(1.4);
7.187(1.5); 7.170(0.6); 7.162(0.6); 7.154(0.4); 4.540(0.8); 4.520(0.7); 4.509(0.7); 3.303(48.5); 3.254(0.7); 3.243(1.0); 2.726(0.4); 2.594(0.3); 2.568(16.0); 2.512(19.5); 2.506(37.8); 2.500(51.0); 2.494(37.8);
2.488(18.5); 2.263(0.9); 2.215(15.7); 2.097(3.3); 1.855(0.8); 1.817(1.2); 1.791(1.2); 1.738(0.6); 1.714(0.6); 1.700(0.8); 1.671(0.7); 1.650(0.8); 1.602(0.7); 1.560(1.2); 1.516(1.1); 1.492(0.9); 1.461(1.0);
1.417(0.5); 0.011(1.9); 0.000(36.3); −0.011(1.6)
Example 377: ¹H-NMR(299.9 MHz, dmso):
δ = 8.136(4.8); 7.780(1.3); 7.451(0.4); 7.421(0.4); 7.356(1.8); 7.351(1.7); 7.331(2.5); 7.326(2.4); 7.310(1.4); 7.303(1.4); 7.284(1.8); 7.278(1.9); 7.220(0.4); 7.204(1.0); 7.185(2.0); 7.180(2.0); 7.161(3.3);
7.154(2.6); 7.135(3.1); 7.129(2.1); 7.118(0.7); 7.111(1.0); 4.518(0.9); 4.494(0.8); 3.732(16.0); 3.653(3.9); 3.301(40.1); 3.277(0.8); 3.266(1.0); 3.233(0.7); 3.222(1.0); 2.495(23.0); 2.489(29.8); 2.483(21.7);
2.444(0.4); 2.295(0.7); 2.260(0.9); 2.251(0.8); 2.115(4.0); 2.072(15.3); 1.868(1.0); 1.829(0.8); 1.708(1.2); 1.667(1.5); 1.630(1.2); 1.594(0.8); 1.535(1.1); 1.500(1.3); 1.456(1.5); 1.419(0.9); −0.012(17.8)
Example 378: ¹H-NMR(299.9 MHz, dmso):
δ = 15.348(0.4); 8.751(5.8); 8.738(6.0); 8.735(5.9); 8.696(0.5); 8.595(0.4); 8.551(4.5); 8.518(4.8); 8.492(0.6); 7.780(0.4); 7.747(5.0); 7.730(5.2); 7.720(5.9); 7.705(5.6); 7.663(0.4); 7.648(0.4); 7.617(0.3);
7.435(5.9); 7.431(6.7); 7.410(13.5); 7.389(5.9); 7.367(1.8); 7.345(5.5); 7.340(5.5); 7.323(16.0); 7.320(12.6); 7.314(13.1); 7.297(3.5); 7.282(6.8); 7.273(4.4); 7.261(4.3); 7.256(5.2); 7.252(4.2); 7.247(3.8);
7.235(2.8); 7.226(2.4); 5.755(2.5); 4.567(2.8); 4.557(2.9); 4.535(3.1); 4.525(2.8); 4.330(0.3); 3.508(0.5); 3.435(0.4); 3.381(0.4); 3.312(294.6); 3.280(3.2); 3.269(4.4); 3.259(2.4); 3.138(0.5); 3.095(0.6);
3.070(0.5); 3.041(0.5); 2.972(0.5); 2.902(0.5); 2.851(0.3); 2.795(0.4); 2.733(1.4); 2.727(1.9); 2.721(1.5); 2.581(1.1); 2.513(99.1); 2.507(202.3); 2.501(274.5); 2.495(194.0); 2.489(87.7); 2.440(1.0);
2.276(1.5); 2.271(2.0); 2.264(1.3); 2.240(0.8); 2.199(2.1); 2.155(2.6); 2.125(0.6); 2.114(1.1); 1.989(0.4); 1.845(2.8); 1.801(3.7); 1.759(5.0); 1.712(2.4); 1.700(1.9); 1.666(1.2); 1.557(5.9); 1.532(4.9);
1.524(5.0); 1.489(4.4); 1.237(0.9); 1.199(0.5); 1.170(0.8); 0.196(1.2); 0.068(0.4); 0.011(10.8); 0.000(280.1); −0.011(9.3); −0.050(0.4); −0.066(0.6); −0.199(1.2)
Example 379: ¹H-NMR(299.9 MHz, cdcl3):
δ = 7.445(0.6); 7.440(0.7); 7.419(0.9); 7.414(0.9); 7.393(1.8); 7.388(2.4); 7.374(1.7); 7.369(2.1); 7.361(2.6); 7.337(1.8); 7.330(1.7); 7.309(3.3); 7.302(1.6); 7.288(2.2); 7.281(2.8); 7.258(46.8); 7.232(0.6);
7.224(0.6); 7.202(0.7); 7.164(16.0); 7.158(11.5); 7.144(4.1); 7.131(1.7); 7.113(0.9); 6.917(1.1); 6.908(5.0); 6.882(7.5); 6.854(4.0); 6.846(1.1); 6.838(1.3); 6.812(1.4); 6.785(0.8); 6.013(1.1); 5.988(1.1);
4.837(1.7); 4.826(1.8); 4.816(1.3); 4.807(1.7); 4.797(1.6); 3.466(1.3); 3.456(1.4); 3.449(1.1); 3.438(1.6); 3.412(1.6); 3.401(1.3); 3.174(0.4); 3.137(0.5); 2.390(0.3); 2.348(0.3); 2.133(1.3); 2.086(2.0);
1.967(1.6); 1.926(2.0); 1.891(1.1); 1.879(2.1); 1.866(1.8); 1.846(2.0); 1.834(3.8); 1.823(2.7); 1.787(6.5); 1.779(5.2); 1.768(4.3); 1.756(3.9); 1.732(1.9); 1.621(0.7); 1.579(1.4); 1.568(1.5); 1.527(39.9);
1.489(2.2); 1.459(1.8); 1.447(2.4); 1.436(1.8); 1.415(1.2); 1.404(1.5); 1.393(1.0); 1.373(0.6); 1.360(0.5); 1.349(0.3); 1.258(0.7); 0.000(45.5)
Example 380: ¹H-NMR(299.9 MHz, dmso):
δ = 8.490(3.0); 8.459(3.2); 8.435(1.6); 8.420(5.3); 8.413(5.8); 8.405(6.2); 8.398(5.4); 8.378(1.7); 8.367(2.3); 8.355(1.6); 7.489(0.4); 7.475(0.6); 7.453(3.3); 7.438(3.9); 7.428(9.0); 7.413(13.4); 7.405(10.5);
7.398(12.3); 7.390(12.9); 7.380(7.6); 7.310(11.3); 7.297(16.0); 7.267(2.3); 7.256(4.5); 7.241(4.6); 7.230(3.6); 7.225(3.2); 7.216(3.9); 7.200(2.6); 7.193(1.7); 7.173(0.8); 7.167(0.7); 4.578(2.3); 4.570(2.4);
4.546(2.3); 4.537(2.3); 4.300(0.6); 4.273(0.6); 3.307(33.7); 3.275(2.2); 3.263(3.3); 3.252(1.8); 3.135(0.5); 3.101(0.7); 3.073(0.5); 2.733(0.3); 2.726(0.4); 2.506(36.1); 2.501(46.9); 2.495(34.3); 2.270(0.5);
2.264(0.5); 2.250(0.7); 2.208(1.9); 2.198(1.8); 2.164(2.0); 2.154(1.8); 2.122(0.8); 2.035(0.8); 2.005(0.8); 1.850(4.1); 1.807(6.0); 1.746(2.4); 1.720(2.2); 1.708(2.0); 1.673(1.6); 1.628(1.6); 1.587(4.9);
1.557(3.5); 1.518(3.2); 1.471(4.6); 1.446(2.8); 1.365(1.2); 1.328(1.4); 1.296(1.2); 1.262(1.5); 1.240(2.8); 1.088(0.4); 0.882(0.9); 0.860(2.2); 0.835(1.2); 0.000(27.8)
Example 381: ¹H-NMR(299.9 MHz, cdcl3):
δ = 7.458(2.2); 7.454(2.1); 7.434(2.9); 7.428(2.8); 7.406(0.5); 7.396(1.6); 7.391(1.5); 7.380(2.0); 7.371(2.4); 7.365(2.9); 7.345(3.6); 7.343(3.8); 7.339(4.3); 7.328(2.0); 7.311(3.4); 7.305(2.7); 7.285(2.0);
7.278(1.8); 7.267(4.5); 7.257(36.0); 7.244(3.6); 7.238(3.0); 7.219(2.0); 7.181(0.4); 7.149(16.0); 7.142(8.0); 7.117(1.4); 6.432(1.0); 6.406(1.0); 4.842(1.5); 4.831(1.6); 4.813(1.5); 4.802(1.5); 3.476(1.1);
3.465(1.4); 3.449(1.2); 3.440(1.0); 3.424(1.5); 3.411(1.1); 2.159(1.3); 2.149(1.1); 2.112(1.8); 1.980(1.4); 1.944(1.8); 1.891(1.1); 1.877(1.4); 1.859(1.5); 1.846(2.2); 1.833(2.3); 1.820(3.3); 1.808(3.8);
1.786(4.9); 1.776(4.1); 1.757(1.6); 1.744(1.8); 1.626(0.5); 1.612(0.5); 1.584(1.1); 1.572(1.1); 1.527(32.4); 1.489(2.2); 1.478(1.4); 1.446(1.1); 1.435(0.8); 1.402(0.3); 1.259(0.4); 0.010(2.4); −0.001(34.7); −0.011
(1.4)
Example 382: ¹H-NMR(299.9 MHz, cdcl3):
δ = 7.809(6.7); 7.783(6.9); 7.761(1.3); 7.735(1.3); 7.712(0.5); 7.698(0.5); 7.604(0.7); 7.564(0.4); 7.493(1.2); 7.466(1.4); 7.409(4.9); 7.387(5.9); 7.380(6.1); 7.348(1.9); 7.319(4.9); 7.291(9.6); 7.258(78.1);
7.229(3.7); 7.201(5.9); 7.182(13.2); 7.167(6.8); 7.159(9.3); 7.151(10.6); 7.142(8.4); 7.120(4.1); 7.065(4.7); 7.039(8.0); 7.026(8.6); 7.021(8.6); 7.001(6.7); 6.964(1.4); 6.906(0.7); 6.892(1.2); 6.862(1.0);
6.255(0.4); 5.823(3.0); 5.798(3.0); 5.532(0.5); 4.816(3.7); 4.805(4.0); 4.786(3.7); 4.776(3.5); 4.356(0.4); 4.329(0.5); 3.642(0.5); 3.610(0.4); 3.484(2.4); 3.473(2.8); 3.452(3.9); 3.431(3.0); 3.419(2.6);
3.198(0.7); 3.153(1.0); 2.409(0.6); 2.372(0.6); 2.231(3.6); 2.186(4.4); 2.082(0.3); 1.937(4.9); 1.866(4.0); 1.821(7.1); 1.808(7.1); 1.783(15.4); 1.776(16.0); 1.761(11.0); 1.750(10.2); 1.627(7.4); 1.586(6.7);
1.552(7.9); 1.526(8.2); 1.483(4.1); 1.438(2.4); 1.395(1.4); 1.259(1.5); 0.902(0.5); 0.883(0.8); 0.854(0.9); 0.832(0.7); 0.196(0.4); 0.000(77.3); −0.199(0.4)
Example 383: ¹H-NMR(299.9 MHz, cdcl3):
δ = 7.652(4.0); 7.644(2.8); 7.635(4.8); 7.631(4.4); 7.622(5.6); 7.611(2.1); 7.602(2.1); 7.597(1.9); 7.581(2.5); 7.555(0.8); 7.514(1.7); 7.496(9.1); 7.480(14.1); 7.467(10.0); 7.450(4.1); 7.445(4.6); 7.438(5.7);
7.423(8.5); 7.416(6.2); 7.408(7.7); 7.401(4.8); 7.392(7.4); 7.379(1.3); 7.360(2.6); 7.356(2.9); 7.334(3.3); 7.330(3.8); 7.315(1.8); 7.312(1.9); 7.291(3.7); 7.257(51.5); 7.227(1.6); 7.207(8.5); 7.193(16.0);
7.181(10.3); 7.175(8.1); 7.164(3.2); 7.159(4.1); 7.149(1.2); 7.134(2.5); 7.122(8.2); 7.108(5.5); 7.099(3.7); 7.091(5.0); 7.084(5.2); 7.071(5.1); 7.054(4.2); 6.931(1.8); 6.916(1.9); 6.911(1.6); 6.901(1.5);
5.815(2.5); 5.789(2.5); 5.536(1.0); 5.508(1.0); 5.294(1.2); 4.834(3.4); 4.823(3.6); 4.813(2.7); 4.804(3.4); 4.793(3.1); 4.394(0.4); 4.383(0.4); 4.358(1.0); 4.346(0.9); 4.326(0.9); 4.315(0.9); 4.289(0.5);
4.277(0.4); 3.475(2.5); 3.464(3.0); 3.438(3.1); 3.421(3.0); 3.410(2.5); 3.197(0.4); 3.179(0.8); 3.169(1.0); 3.142(1.5); 3.131(1.7); 3.104(0.7); 3.092(0.7); 2.365(1.1); 2.356(1.2); 2.321(1.2); 2.314(1.2);
2.124(2.8); 2.080(3.9); 2.071(3.6); 2.039(1.0); 2.023(1.3); 1.980(2.0); 1.942(3.5); 1.931(3.2); 1.901(4.4); 1.881(4.0); 1.868(5.0); 1.855(2.9); 1.835(3.8); 1.823(5.8); 1.811(3.8); 1.778(9.1); 1.770(9.7);
1.748(9.5); 1.714(5.0); 1.703(3.8); 1.669(1.1); 1.658(1.1); 1.639(0.7); 1.628(0.8); 1.604(1.6); 1.584(2.2); 1.576(2.2); 1.562(3.2); 1.533(34.3); 1.510(3.6); 1.499(3.6); 1.489(3.4); 1.459(4.6); 1.429(4.0);
1.418(4.8); 1.406(2.7); 1.387(3.3); 1.375(3.2); 1.358(2.0); 1.345(1.7); 1.331(1.1); 1.316(1.0); 1.302(0.8); 1.257(1.2); 0.882(0.7); 0.858(0.4); 0.000(49.2); −0.011(2.1)
Example 384: ¹H-NMR(299.9 MHz, dmso):
δ = 8.272(5.0); 7.644(1.8); 7.618(2.1); 7.543(1.0); 7.517(2.6); 7.499(1.4); 7.474(1.9); 7.450(2.1); 7.417(1.4); 7.353(1.1); 7.328(1.6); 7.303(0.7); 4.419(0.9); 4.393(0.9); 3.781(16.0); 3.317(16.6); 3.164(0.9);
3.123(0.9); 2.507(10.1); 2.501(12.8); 2.495(9.4); 2.348(0.7); 2.307(0.7); 2.296(0.7); 2.265(0.3); 2.084(16.0); 1.883(0.8); 1.842(0.9); 1.725(0.7); 1.680(3.2); 1.636(0.8); 1.584(0.6); 1.558(0.7); 1.537(0.8);
1.482(1.0); 1.438(1.3); 1.391(0.5); 0.000(6.7)
Example 385: ¹H-NMR(299.9 MHz, dmso):
δ = 8.792(7.7); 8.775(11.8); 8.740(6.0); 7.808(5.7); 7.792(5.9); 7.782(7.7); 7.766(7.3); 7.715(9.2); 7.688(13.6); 7.658(6.1); 7.612(9.2); 7.587(16.0); 7.562(6.3); 7.477(5.1); 7.452(7.9); 7.426(3.6); 5.757(0.4);
4.392(4.0); 4.368(3.8); 3.316(121.2); 3.214(3.7); 3.173(4.2); 2.728(0.5); 2.513(27.2); 2.507(54.0); 2.501(72.8); 2.495(54.6); 2.272(1.5); 2.227(2.8); 2.194(3.1); 2.151(1.2); 1.844(3.4); 1.802(5.2); 1.756(5.4);
1.705(3.1); 1.685(2.8); 1.674(2.9); 1.649(2.3); 1.611(3.9); 1.580(7.9); 1.503(4.4); 1.458(5.0); 1.419(2.3); 1.404(2.3); 1.236(1.1); 0.011(2.1); 0.000(43.5)
Example 386: ¹H-NMR(299.9 MHz, dmso):
δ = 8.856(4.9); 8.823(5.0); 8.362(0.4); 8.326(0.4); 7.688(7.2); 7.662(8.3); 7.624(0.8); 7.586(2.7); 7.563(16.0); 7.540(7.6); 7.511(5.0); 7.505(3.6); 7.482(6.4); 7.460(3.5); 7.454(4.3); 7.432(5.2); 7.403(5.6);
7.378(2.8); 7.350(0.9); 7.325(0.4); 7.160(2.0); 7.149(10.2); 7.123(13.8); 7.096(8.2); 7.085(1.7); 7.014(0.8); 6.989(1.0); 6.962(0.6); 6.639(0.4); 4.410(3.7); 4.385(3.6); 3.317(113.9); 3.187(3.5); 3.145(3.8);
2.891(0.3); 2.728(0.5); 2.507(48.1); 2.501(63.4); 2.495(47.2); 2.277(1.1); 2.237(2.6); 2.203(2.8); 2.162(1.0); 1.989(0.4); 1.845(3.2); 1.802(4.3); 1.750(4.9); 1.706(3.6); 1.687(3.4); 1.644(4.1); 1.615(4.0);
1.585(5.7); 1.482(4.4); 1.442(5.8); 1.409(2.1); 1.318(0.5); 1.290(0.8); 1.270(1.0); 1.251(1.3); 1.240(1.4); 0.881(1.1); 0.860(2.9); 0.836(1.3); 0.011(2.1); 0.000(34.3)
Example 387: ¹H-NMR(299.9 MHz, dmso):
δ = 8.722(7.1); 8.689(7.2); 8.462(8.8); 8.455(9.4); 8.447(9.4); 8.440(8.9); 8.356(0.9); 8.349(1.0); 8.340(1.0); 8.333(0.9); 8.279(0.8); 8.248(0.7); 7.759(0.4); 7.738(1.1); 7.701(9.5); 7.675(11.5); 7.664(4.5);
7.638(9.8); 7.613(8.8); 7.568(13.7); 7.555(7.7); 7.548(10.5); 7.530(16.0); 7.523(14.2); 7.501(14.7); 7.486(13.7); 7.476(6.6); 7.461(7.4); 7.451(6.5); 7.426(9.4); 7.401(4.3); 7.369(1.8); 7.360(1.8); 7.344(1.7);
7.305(1.3); 7.298(1.4); 7.280(0.8); 7.273(0.8); 4.401(5.2); 4.376(4.9); 3.316(93.4); 3.207(4.8); 3.164(5.2); 2.945(0.4); 2.908(0.5); 2.728(0.5); 2.507(41.8); 2.501(54.4); 2.495(40.4); 2.272(1.6); 2.231(3.6);
2.196(3.7); 2.155(1.4); 1.941(0.6); 1.850(7.1); 1.814(11.2); 1.751(2.0); 1.740(2.3); 1.731(2.3); 1.697(5.2); 1.664(7.0); 1.625(6.3); 1.590(7.4); 1.490(6.3); 1.447(7.7); 1.408(2.9); 1.293(0.8); 1.240(1.1);
0.881(0.5); 0.859(1.4); 0.836(0.6); 0.000(27.7)
Example 388: ¹H-NMR(299.9 MHz, dmso):
δ = 8.605(5.2); 8.573(5.2); 7.697(7.4); 7.671(9.2); 7.656(2.4); 7.629(6.7); 7.604(15.6); 7.576(3.2); 7.474(2.6); 7.467(4.2); 7.447(12.1); 7.442(16.0); 7.434(7.6); 7.417(12.8); 7.411(9.6); 7.397(10.0);
7.389(7.8); 7.373(8.0); 7.367(7.0); 7.349(3.0); 7.343(2.5); 7.322(0.6); 7.311(0.6); 7.233(0.4); 7.108(7.6); 7.103(6.4); 7.085(7.1); 7.078(6.2); 6.829(0.4); 4.419(3.9); 4.389(3.8); 3.317(69.4); 3.196(3.7);
3.155(4.0); 2.727(0.4); 2.506(38.5); 2.500(50.4); 2.494(37.8); 2.336(1.1); 2.290(2.9); 2.255(2.9); 2.212(1.0); 1.830(5.4); 1.799(7.6); 1.744(1.7); 1.692(4.8); 1.666(6.0); 1.637(3.5); 1.569(4.0); 1.508(1.0);
1.464(4.7); 1.425(5.9); 1.382(2.2); 1.291(1.1); 1.268(1.3); 1.249(1.8); 1.241(2.0); 0.881(1.8); 0.859(4.4); 0.835(1.9); −0.001(26.1)
Example 389: ¹H-NMR(299.9 MHz, dmso):
δ = 8.547(5.5); 8.514(5.8); 8.075(3.6); 8.043(3.7); 7.846(8.1); 7.820(8.9); 7.755(5.4); 7.731(7.2); 7.711(6.2); 7.699(8.4); 7.672(10.0); 7.663(7.8); 7.648(6.0); 7.636(7.3); 7.623(8.5); 7.605(13.5); 7.580(6.2);
7.455(8.4); 7.430(16.0); 7.405(8.6); 7.388(3.4); 7.362(4.6); 7.338(2.1); 7.299(2.7); 7.276(5.7); 7.251(3.5); 7.169(4.4); 7.164(4.9); 7.143(7.3); 7.138(7.9); 7.118(3.8); 7.112(3.7); 7.069(2.9); 7.063(3.1);
7.044(4.5); 7.038(4.8); 7.018(2.3); 7.012(2.3); 6.953(7.9); 6.948(8.1); 6.928(7.3); 6.923(7.1); 6.580(4.6); 6.575(4.9); 6.555(4.4); 6.550(4.5); 5.754(0.7); 4.392(5.2); 4.366(5.0); 3.313(160.7); 3.199(3.6);
3.156(3.8); 2.959(1.3); 2.923(2.4); 2.886(1.3); 2.726(0.6); 2.506(59.8); 2.500(81.2); 2.494(61.8); 2.349(1.1); 2.303(2.8); 2.269(3.1); 2.226(1.0); 1.941(4.9); 1.903(3.6); 1.833(6.0); 1.796(6.3); 1.746(2.8);
1.691(6.2); 1.652(4.5); 1.600(3.1); 1.548(5.7); 1.518(5.2); 1.458(6.7); 1.421(7.0); 1.385(2.6); 1.333(1.4); 1.287(1.8); 1.247(1.7); 1.235(1.7); 0.010(2.0); −0.001(41.3)
Example 390: ¹H-NMR(299.9 MHz, dmso):
δ = 8.657(5.2); 8.625(5.3); 7.750(7.5); 7.724(15.3); 7.709(11.1); 7.697(7.3); 7.683(16.0); 7.657(7.7); 7.641(14.7); 7.617(9.5); 7.593(2.7); 7.472(4.2); 7.447(6.3); 7.423(2.9); 7.176(7.2); 7.151(7.0);
5.755(1.3); 4.398(3.7); 4.373(3.5); 3.315(171.9); 3.206(3.5); 3.164(3.9); 2.726(0.7); 2.506(66.4); 2.500(90.4); 2.495(70.3); 2.353(1.1); 2.303(2.8); 2.270(3.3); 2.227(1.1); 1.828(3.1); 1.784(4.7); 1.745(4.7);
1.707(2.3); 1.685(2.6); 1.655(3.3); 1.626(4.4); 1.594(4.2); 1.585(4.2); 1.564(5.7); 1.478(4.0); 1.431(5.4); 1.385(2.0); 1.235(0.9); −0.001(43.8)
Example 391: ¹H-NMR(299.9 MHz, dmso):
δ = 8.413(5.2); 8.382(5.3); 8.179(1.0); 8.151(1.2); 8.103(1.1); 8.075(1.2); 7.758(0.7); 7.734(1.1); 7.707(0.7); 7.575(0.8); 7.550(1.1); 7.524(0.7); 7.456(2.0); 7.434(4.0); 7.428(4.0); 7.406(7.5); 7.380(10.2);
7.366(8.2); 7.357(11.9); 7.349(12.0); 7.341(6.4); 7.334(8.9); 7.252(1.6); 7.245(3.0); 7.227(9.2); 7.220(14.8); 7.208(14.6); 7.196(11.3); 7.188(6.3); 7.171(1.9); 7.164(1.2); 7.063(2.4); 7.053(11.7);
7.027(16.0); 7.000(9.3); 6.989(1.8); 6.640(3.0); 4.830(1.5); 4.821(1.8); 4.799(4.1); 4.789(3.0); 4.777(4.1); 4.755(1.8); 4.746(1.4); 3.547(2.5); 3.525(4.9); 3.505(4.9); 3.485(4.8); 3.464(2.5); 3.320(45.6);
2.728(0.4); 2.507(40.9); 2.501(54.2); 2.496(40.9); 2.246(1.2); 2.219(2.3); 2.206(2.7); 2.194(3.5); 2.178(3.7); 2.139(3.2); 2.109(2.1); 2.099(4.1); 2.070(3.0); 2.059(2.0); 2.030(1.4); 1.949(1.1); 1.921(2.1);
1.887(5.3); 1.866(6.7); 1.844(5.0); 1.830(3.4); 1.806(1.7); 1.712(1.9); 1.701(3.2); 1.691(3.5); 1.663(5.3); 1.647(6.6); 1.625(3.9); 1.604(1.5); 1.565(0.4); 1.236(0.6); 0.001(27.2)
Example 392: ¹H-NMR(299.9 MHz, dmso):
δ = 8.092(1.2); 8.059(1.2); 7.650(1.7); 7.625(2.5); 7.610(1.8); 7.587(1.0); 7.570(0.5); 7.546(0.9); 7.522(1.7); 7.502(4.3); 7.483(0.6); 7.381(0.8); 7.361(1.2); 7.343(0.9); 7.318(0.7); 4.368(0.9); 4.345(1.0);
3.295(22.7); 3.168(0.8); 3.127(0.9); 2.936(0.4); 2.583(14.4); 2.486(16.3); 2.481(15.2); 2.475(10.3); 2.265(16.0); 1.983(6.5); 1.837(1.0); 1.795(1.4); 1.753(1.5); 1.686(1.3); 1.654(1.2); 1.583(0.7); 1.539(1.2);
1.468(1.1); 1.413(1.3); −0.020(7.7)
Example 393: ¹H-NMR(299.9 MHz, dmso):
δ = 8.155(4.8); 7.345(1.4); 7.312(1.4); 7.258(0.3); 7.236(0.7); 7.230(0.7); 7.208(1.4); 7.186(0.8); 7.181(1.0); 7.159(0.4); 6.957(2.0); 6.927(2.9); 6.898(1.6); 4.443(0.8); 4.434(0.8); 4.410(0.8); 4.401(0.8);
3.829(0.4); 3.761(16.0); 3.694(0.4); 3.683(0.3); 3.383(1.3); 3.316(59.9); 3.266(0.7); 3.249(1.9); 3.221(0.7); 3.211(1.1); 3.200(0.6); 2.568(0.3); 2.537(0.4); 2.506(7.6); 2.500(10.0); 2.494(7.8); 2.462(0.6);
2.451(0.6); 2.440(0.5); 2.434(0.5); 2.188(0.4); 2.120(15.5); 2.053(0.4); 2.044(0.3); 1.849(0.8); 1.831(0.6); 1.810(1.2); 1.798(1.1); 1.778(0.6); 1.761(0.8); 1.750(0.9); 1.737(0.9); 1.706(1.5); 1.669(2.2);
1.626(0.9); 1.525(0.7); 1.494(0.7); 1.399(0.6); 1.359(0.5); 1.235(0.4); 0.010(0.4); −0.001(6.9)
Example 394: ¹H-NMR(299.9 MHz, cdcl3):
δ = 7.271(0.4); 7.269(0.5); 7.260(14.3); 7.166(0.6); 7.159(0.6); 7.145(0.4); 7.139(1.3); 7.132(0.5); 7.116(0.6); 7.111(0.8); 7.089(0.4); 6.857(1.7); 6.827(2.4); 6.798(1.3); 6.370(0.5); 6.345(0.5); 4.435(0.6);
4.424(0.7); 4.409(0.7); 4.398(0.6); 3.440(0.5); 3.428(0.9); 3.417(0.5); 3.394(0.5); 3.383(0.9); 3.372(0.5); 2.663(16.0); 2.609(13.3); 2.241(0.3); 2.230(0.4); 2.198(0.4); 2.186(0.4); 2.142(0.7); 2.099(0.7);
1.956(0.4); 1.945(0.6); 1.925(0.5); 1.915(0.5); 1.885(0.8); 1.836(0.5); 1.769(0.4); 1.758(0.4); 1.725(1.1); 1.692(0.9); 1.679(0.6); 1.666(0.3); 1.557(2.8); 1.544(2.2); 1.480(0.7); 1.448(1.3); 1.410(0.6);
0.010(0.8); −0.001(16.7); −0.011(0.7)
Example 395: ¹H-NMR(299.9 MHz, dmso):
δ = 8.826(5.7); 8.793(5.8); 8.533(0.4); 8.500(0.4); 7.497(1.9); 7.475(4.1); 7.470(4.0); 7.447(7.6); 7.425(4.3); 7.419(5.0); 7.397(2.5); 7.348(0.4); 7.316(1.8); 7.294(3.8); 7.289(3.8); 7.267(7.2); 7.246(4.3);
7.239(4.8); 7.218(2.5); 7.116(2.7); 7.106(12.1); 7.080(16.0); 7.053(9.9); 6.993(10.8); 6.964(16.0); 6.935(8.5); 6.879(0.4); 6.639(0.4); 4.508(4.4); 4.478(4.3); 3.314(72.7); 3.276(3.4); 3.266(5.4); 3.256(3.6);
3.232(3.8); 3.221(5.6); 3.020(1.7); 3.001(0.6); 2.996(0.6); 2.978(0.5); 2.868(0.8); 2.727(0.6); 2.507(55.3); 2.501(71.5); 2.495(54.6); 2.431(3.2); 2.421(3.2); 2.387(3.1); 2.377(3.0); 2.344(1.3); 2.271(0.7);
1.912(0.5); 1.898(0.5); 1.778(8.0); 1.753(11.9); 1.716(7.3); 1.667(8.2); 1.623(4.2); 1.559(1.1); 1.514(4.5); 1.380(2.9); 1.337(2.6); 1.236(1.1); 1.216(0.7); 1.193(0.5); 1.174(0.6); 1.151(0.4); 1.006(0.5);
0.981(1.0); 0.957(0.5); 0.000(41.7)
Example 396: ¹H-NMR(299.9 MHz, dmso):
δ = 8.544(5.4); 8.511(5.7); 7.475(0.4); 7.444(1.8); 7.437(3.5); 7.417(11.7); 7.412(16.0); 7.395(10.8); 7.389(9.7); 7.378(8.3); 7.369(7.5); 7.363(6.2); 7.354(8.8); 7.346(7.5); 7.330(4.5); 7.306(3.9); 7.301(3.8);
7.278(7.0); 7.251(4.7); 7.229(2.2); 7.148(7.9); 7.126(7.2); 7.119(6.3); 7.034(9.1); 7.004(14.1); 6.975(7.7); 6.920(0.7); 6.895(0.5); 6.638(0.5); 5.822(0.4); 4.508(4.0); 4.477(4.1); 3.313(83.1); 3.284(4.1);
3.273(5.4); 3.262(3.7); 3.238(3.7); 3.228(5.4); 3.177(0.3); 3.027(0.6); 3.007(1.3); 2.984(1.4); 2.966(0.9); 2.726(0.6); 2.505(60.0); 2.500(75.1); 2.494(57.2); 2.457(3.4); 2.448(3.3); 2.413(3.1); 2.404(2.9);
2.370(1.2); 2.308(1.8); 2.276(0.9); 2.210(0.4); 1.790(9.1); 1.757(15.2); 1.656(4.1); 1.612(3.6); 1.564(0.8); 1.476(4.1); 1.374(2.7); 1.335(2.4); 1.297(1.0); 1.234(1.2); 0.996(1.0); 0.972(1.9); 0.948(0.8); −0.001
(31.9)
Example 397: ¹H-NMR(299.9 MHz, dmso):
δ = 8.725(5.0); 8.691(5.0); 8.439(9.3); 8.432(10.4); 8.423(10.1); 8.417(9.7); 8.383(0.4); 8.374(0.4); 8.370(0.5); 8.361(0.4); 7.559(6.8); 7.552(7.5); 7.534(14.9); 7.527(13.5); 7.492(14.7); 7.477(13.8);
7.467(7.4); 7.452(7.4); 7.394(0.9); 7.388(0.9); 7.380(1.4); 7.336(1.6); 7.314(3.4); 7.307(2.9); 7.293(2.7); 7.286(7.0); 7.280(3.1); 7.264(3.3); 7.259(4.5); 7.237(2.1); 7.043(9.8); 7.012(13.3); 6.984(7.5);
4.490(3.4); 4.462(3.2); 3.366(0.4); 3.315(150.1); 3.295(3.5); 3.284(4.9); 3.273(3.0); 3.250(3.1); 3.240(4.8); 3.229(2.6); 3.005(0.7); 2.809(0.5); 2.728(0.5); 2.514(21.5); 2.508(44.2); 2.501(60.6); 2.495(44.1);
2.489(20.7); 2.468(1.0); 2.457(1.0); 2.422(2.1); 2.412(2.2); 2.379(2.2); 2.368(2.2); 2.334(0.9); 2.323(0.8); 2.271(0.5); 1.878(0.3); 1.796(4.6); 1.757(16.0); 1.726(4.5); 1.678(3.6); 1.631(2.8); 1.521(3.8);
1.425(1.2); 1.392(2.2); 1.353(2.0); 1.293(0.8); 1.270(0.7); 1.250(0.9); 1.236(1.2); 1.226(0.7); 1.201(0.3); 0.980(0.5); 0.882(0.9); 0.860(2.9); 0.836(1.1); 0.011(1.4); 0.009(1.0); 0.001(36.2); −0.010(1.4)
Example 398: ¹H-NMR(299.9 MHz, cdcl3):
δ = 8.760(6.4); 8.748(6.4); 8.680(0.4); 8.662(0.4); 7.660(5.4); 7.637(7.9); 7.606(0.3); 7.541(7.5); 7.526(7.4); 7.515(5.3); 7.500(4.9); 7.435(0.4); 7.420(0.4); 7.410(0.6); 7.394(0.6); 7.349(0.7); 7.325(0.5);
7.260(40.8); 7.246(2.1); 7.224(3.3); 7.217(3.6); 7.196(6.9); 7.190(3.3); 7.174(3.5); 7.169(4.5); 7.147(2.1); 6.895(9.8); 6.865(16.0); 6.837(8.2); 6.394(3.1); 6.367(3.4); 4.546(3.7); 4.535(4.0); 4.517(3.9);
4.506(3.6); 3.461(2.8); 3.450(5.2); 3.438(3.1); 3.416(3.1); 3.405(5.5); 3.393(3.0); 2.755(1.2); 2.203(0.8); 2.190(1.0); 2.161(5.3); 2.116(6.9); 2.072(1.6); 2.060(1.3); 1.916(4.1); 1.901(3.8); 1.885(3.6);
1.851(5.3); 1.823(3.5); 1.806(4.9); 1.793(5.0); 1.779(5.4); 1.767(6.3); 1.732(6.6); 1.683(1.5); 1.642(1.2); 1.607(1.0); 1.597(1.1); 1.547(1.3); 1.532(1.4); 1.488(4.4); 1.455(7.0); 1.414(3.8); 1.371(1.0);
1.338(0.8); 1.325(0.7); 1.314(0.7); 1.292(1.0); 1.273(0.9); 1.257(1.0); 1.115(0.3); 1.091(0.5); 0.903(0.8); 0.881(1.9); 0.858(0.9); −0.001(43.6); −0.011(2.4)
Example 399: ¹H-NMR(299.9 MHz, dmso):
δ = 8.618(4.2); 8.585(4.3); 7.726(7.8); 7.701(16.0); 7.677(5.2); 7.652(0.8); 7.624(5.0); 7.598(5.5); 7.574(2.3); 7.545(0.4); 7.347(1.3); 7.325(2.7); 7.320(2.4); 7.298(5.3); 7.275(2.6); 7.270(3.5); 7.249(1.6);
7.213(6.2); 7.188(5.8); 7.060(7.6); 7.029(11.1); 7.001(6.2); 4.487(2.8); 4.454(2.8); 3.314(59.9); 3.292(3.1); 3.280(3.9); 3.270(2.4); 3.246(2.6); 3.236(4.0); 3.225(2.1); 3.015(0.4); 3.007(0.4); 2.726(0.4);
2.512(20.2); 2.506(39.1); 2.500(52.2); 2.494(38.3); 2.488(18.9); 2.461(2.0); 2.427(2.0); 2.417(1.9); 2.384(0.7); 2.371(0.6); 2.270(0.4); 1.792(3.9); 1.763(7.4); 1.741(5.6); 1.729(6.3); 1.670(5.2); 1.626(2.6);
1.476(2.7); 1.377(1.8); 1.336(1.6); 1.235(0.6); 0.978(0.6); 0.010(1.4); −0.001(26.9); −0.011(1.0)
Example 400: ¹H-NMR(299.9 MHz, dmso):
δ = 8.485(4.1); 8.452(4.1); 8.236(0.3); 8.205(0.3); 7.826(6.4); 7.823(6.2); 7.799(7.0); 7.797(6.4); 7.774(0.6); 7.772(0.6); 7.748(0.6); 7.746(0.6); 7.438(3.5); 7.435(3.3); 7.413(7.6); 7.410(6.9); 7.388(4.6);
7.385(4.0); 7.342(0.5); 7.338(0.5); 7.327(1.2); 7.317(1.1); 7.313(1.2); 7.306(2.6); 7.300(2.4); 7.292(1.4); 7.278(4.9); 7.257(2.8); 7.251(3.2); 7.229(1.6); 7.211(0.4); 7.185(0.4); 7.180(0.4); 7.148(3.7);
7.143(4.1); 7.123(5.8); 7.117(6.1); 7.097(3.3); 7.091(3.3); 7.061(1.7); 7.048(7.1); 7.017(16.0); 6.991(10.9); 6.962(0.7); 6.713(0.5); 6.708(0.5); 6.688(0.5); 6.683(0.4); 6.638(0.4); 4.482(2.6); 4.474(2.6);
4.449(2.6); 4.442(2.4); 3.315(8.7); 3.285(2.8); 3.274(3.8); 3.263(2.4); 3.240(2.5); 3.229(3.7); 3.219(2.1); 3.035(0.5); 3.011(1.0); 2.992(1.1); 2.969(0.7); 2.511(9.3); 2.506(16.9); 2.500(21.7); 2.494(16.0);
2.472(2.2); 2.461(2.0); 2.427(1.9); 2.417(1.9); 2.375(2.8); 2.236(0.3); 1.900(0.5); 1.887(0.7); 1.857(1.7); 1.813(4.7); 1.782(9.6); 1.732(2.6); 1.719(1.8); 1.686(0.7); 1.645(2.6); 1.596(2.6); 1.546(0.7);
1.510(2.3); 1.471(2.5); 1.422(1.2); 1.378(2.0); 1.336(1.9); 1.296(0.7); 1.249(0.4); 1.234(0.5); 1.225(0.5); 0.998(0.8); 0.974(1.6); 0.950(0.8); 0.010(0.7); −0.001(11.5); −0.012(0.6)
Example 401: ¹H-NMR(299.9 MHz, dmso):
δ = 7.895(0.3); 7.856(1.2); 7.824(1.1); 7.567(1.9); 7.542(2.3); 7.359(0.3); 7.331(0.6); 7.324(0.4); 7.294(2.1); 7.287(2.7); 7.276(5.2); 7.261(0.5); 7.148(1.0); 7.137(0.9); 7.129(0.9); 7.121(1.2); 7.110(1.0);
7.104(0.8); 7.091(0.7); 4.554(0.7); 4.547(0.8); 4.524(0.7); 3.316(88.2); 3.246(0.6); 3.236(0.9); 3.226(0.6); 3.204(0.6); 3.192(1.0); 3.182(0.7); 3.011(0.4); 2.988(0.4); 2.735(0.3); 2.729(0.4); 2.592(0.9);
2.573(15.5); 2.546(1.3); 2.515(21.1); 2.509(40.5); 2.503(54.3); 2.497(39.9); 2.491(19.5); 2.314(0.4); 2.305(0.4); 2.272(1.1); 2.224(16.0); 2.187(0.4); 2.093(4.0); 1.906(0.4); 1.856(0.9); 1.799(1.4);
1.725(0.5); 1.686(0.8); 1.644(1.0); 1.600(0.8); 1.557(1.3); 1.531(1.3); 1.487(1.1); 1.453(0.7); 1.413(0.5); 1.237(0.5); 0.999(0.4); 0.976(0.7); 0.952(0.4); 0.013(1.1); 0.002(24.8); −0.009(1.2)
Example 402: ¹H-NMR(299.9 MHz, dmso):
δ = 8.154(5.0); 7.539(1.9); 7.512(2.1); 7.310(1.0); 7.304(1.1); 7.284(2.3); 7.279(2.3); 7.256(1.5); 7.233(2.0); 7.209(0.9); 7.186(1.4); 7.154(1.5); 7.097(1.1); 7.091(1.2); 7.071(1.7); 7.047(0.9); 7.041(0.8);
4.531(0.9); 4.504(0.9); 3.745(16.0); 3.664(0.6); 3.314(52.3); 3.226(1.0); 3.181(1.1); 2.506(29.1); 2.500(38.1); 2.495(28.7); 2.310(0.7); 2.276(0.9); 2.234(0.3); 2.128(0.7); 2.088(15.3); 1.877(0.8); 1.836(0.9);
1.727(1.0); 1.688(0.8); 1.666(1.2); 1.631(1.3); 1.601(1.0); 1.528(1.4); 1.483(1.2); 0.000(11.8)
Example 403: ¹H-NMR(299.9 MHz, dmso):
δ = 11.744(0.5); 8.752(8.5); 8.737(9.2); 8.711(4.7); 8.694(4.6); 8.557(7.0); 8.526(10.3); 8.500(4.2); 8.019(0.7); 7.749(6.9); 7.733(7.3); 7.722(9.1); 7.707(8.7); 7.687(3.7); 7.671(3.4); 7.661(4.2); 7.645(4.1);
7.606(10.7); 7.592(5.8); 7.582(12.1); 7.579(12.5); 7.567(6.3); 7.421(10.3); 7.396(10.4); 7.379(16.0); 7.359(10.4); 7.339(6.1); 7.309(12.5); 7.303(15.1); 7.283(8.4); 7.277(7.9); 7.206(6.8); 7.200(6.5);
7.174(10.4); 7.155(6.1); 7.149(7.9); 7.122(3.1); 7.116(3.0); 6.933(0.8); 6.908(0.6); 6.819(0.5); 6.637(0.6); 5.754(5.1); 4.570(4.4); 4.560(4.7); 4.539(4.7); 4.530(4.5); 4.310(1.5); 4.276(1.6); 3.506(0.6);
3.395(0.8); 3.380(1.3); 3.313(680.3); 3.272(6.5); 3.261(7.7); 3.250(5.7); 3.227(5.1); 3.217(7.2); 3.176(1.7); 3.158(1.4); 3.109(1.8); 3.084(1.8); 3.057(3.0); 3.021(2.4); 2.991(1.7); 2.876(0.6); 2.726(2.7);
2.619(0.7); 2.512(136.9); 2.506(261.4); 2.500(344.3); 2.494(251.5); 2.270(3.2); 2.232(2.0); 2.198(3.9); 2.156(4.5); 2.116(2.2); 2.033(2.6); 2.001(2.1); 1.923(2.5); 1.805(8.9); 1.772(8.8); 1.733(5.5);
1.696(4.2); 1.653(2.8); 1.554(11.7); 1.492(7.6); 1.458(6.6); 1.425(6.7); 1.396(4.1); 1.350(3.0); 1.311(3.2); 1.236(3.7); 0.996(0.9); 0.974(1.9); 0.950(0.9); 0.855(0.7); 0.194(0.5); 0.010(8.0); −0.001(108.3)
Example 404: ¹H-NMR(299.9 MHz, dmso):
δ = 8.654(0.4); 8.613(3.9); 8.582(4.0); 8.533(1.5); 7.577(7.3); 7.551(8.0); 7.534(2.6); 7.530(2.8); 7.495(1.2); 7.473(3.0); 7.466(2.8); 7.444(5.8); 7.422(3.7); 7.417(4.1); 7.399(2.6); 7.372(2.3); 7.348(3.1);
7.323(2.1); 7.289(15.2); 7.275(16.0); 7.248(1.0); 7.170(0.9); 7.158(4.1); 7.143(5.1); 7.131(5.6); 7.114(5.5); 7.101(11.8); 7.075(11.3); 7.048(9.9); 7.021(4.1); 6.994(2.7); 4.560(2.8); 4.537(2.7); 4.327(0.8);
4.304(0.8); 4.267(0.5); 3.393(0.4); 3.387(0.4); 3.314(277.9); 3.243(3.6); 3.232(2.4); 3.209(2.5); 3.199(3.7); 3.188(2.2); 3.120(0.5); 3.061(0.7); 3.036(1.3); 2.996(0.7); 2.732(1.3); 2.726(1.4); 2.581(0.6);
2.512(75.7); 2.506(153.9); 2.500(210.7); 2.494(155.5); 2.488(75.3); 2.395(0.4); 2.364(0.5); 2.276(1.4); 2.270(1.8); 2.218(2.4); 2.176(2.4); 2.132(1.2); 2.086(0.4); 2.017(1.2); 1.930(1.0); 1.895(1.2);
1.800(4.5); 1.771(4.6); 1.714(3.0); 1.687(2.1); 1.675(2.1); 1.648(1.8); 1.613(2.1); 1.570(5.7); 1.544(3.7); 1.520(3.5); 1.459(4.1); 1.421(3.4); 1.341(1.1); 1.301(0.9); 1.261(1.0); 1.234(1.5); 1.201(0.7);
1.178(0.6); 1.156(0.5); 0.010(4.8); 0.000(116.1); −0.011(5.0); −0.064(0.4); −0.199(0.5)
Example 405: ¹H-NMR(299.9 MHz, dmso):
δ = 8.474(0.4); 8.370(3.6); 8.339(3.7); 8.018(0.6); 7.666(0.4); 7.588(6.1); 7.563(7.4); 7.516(0.7); 7.426(2.9); 7.406(7.8); 7.400(12.2); 7.378(6.6); 7.372(7.1); 7.346(8.8); 7.342(9.3); 7.320(16.0); 7.310(6.9);
7.303(3.5); 7.293(3.7); 7.287(3.0); 7.179(3.6); 7.170(3.1); 7.152(4.0); 7.144(3.1); 7.132(2.5); 7.123(2.4); 6.977(5.6); 6.953(5.3); 6.947(5.2); 4.580(2.7); 4.549(2.5); 3.928(0.5); 3.716(0.4); 3.507(0.7);
3.420(0.4); 3.381(0.9); 3.314(302.3); 3.249(3.3); 3.215(2.6); 3.205(3.5); 2.991(0.5); 2.726(1.7); 2.700(0.6); 2.512(71.1); 2.506(138.4); 2.500(185.4); 2.494(136.2); 2.489(66.4); 2.445(0.7); 2.317(1.0);
2.270(3.3); 2.263(2.8); 2.228(2.4); 2.185(1.0); 1.838(3.7); 1.804(5.0); 1.729(1.4); 1.693(2.2); 1.666(2.4); 1.641(2.5); 1.596(2.5); 1.558(4.1); 1.498(2.9); 1.448(3.4); 1.408(1.6); 1.397(1.6); 1.329(0.4);
1.263(0.6); 1.234(1.3); 1.180(0.3); 1.001(0.3); 0.978(0.6); 0.956(0.4); 0.195(0.4); 0.000(67.1); −0.011(2.8); −1.283(0.9)
Example 406: ¹H-NMR(299.9 MHz, dmso):
δ = 8.326(5.2); 8.294(5.4); 8.269(2.0); 8.238(1.7); 7.977(0.5); 7.951(0.5); 7.922(0.3); 7.895(0.4); 7.809(8.3); 7.783(11.4); 7.756(3.0); 7.689(0.5); 7.649(0.6); 7.589(8.0); 7.563(9.3); 7.545(3.2); 7.476(0.5);
7.459(0.9); 7.427(1.8); 7.400(3.3); 7.390(6.6); 7.368(16.0); 7.365(16.0); 7.342(14.7); 7.328(13.2); 7.302(5.9); 7.281(2.3); 7.214(0.6); 7.185(4.5); 7.178(4.3); 7.158(5.9); 7.132(6.4); 7.127(7.7); 7.106(7.8);
7.101(8.3); 7.087(3.2); 7.081(4.2); 7.076(4.2); 7.067(2.9); 7.061(2.9); 7.041(1.3); 7.036(1.4); 6.795(7.5); 6.790(7.9); 6.770(8.6); 6.765(9.0); 6.743(2.2); 4.546(3.3); 4.520(3.3); 4.514(3.3); 4.275(0.7);
4.263(0.7); 3.376(0.3); 3.315(139.6); 3.256(4.2); 3.223(3.0); 3.212(4.4); 3.100(0.7); 3.072(1.4); 3.062(1.2); 3.034(0.9); 3.021(0.8); 2.727(0.9); 2.506(93.6); 2.500(128.9); 2.495(98.8); 2.319(1.0); 2.276(3.8);
2.243(3.1); 2.231(2.8); 2.199(1.2); 2.137(0.4); 2.045(1.1); 1.987(0.6); 1.897(4.8); 1.839(3.5); 1.798(4.1); 1.724(2.1); 1.691(4.2); 1.661(5.6); 1.634(3.4); 1.607(2.5); 1.556(3.8); 1.491(5.1); 1.453(6.1);
1.399(2.5); 1.356(1.3); 1.278(1.2); 1.238(1.3); 1.176(0.6); 1.149(0.4); 1.097(0.4); 1.073(0.4); 1.049(0.3); 1.002(0.4); 0.977(0.6); 0.859(0.4); 0.010(1.9); 0.000(38.6); −1.283(0.7)
Example 407: ¹H-NMR(299.9 MHz, dmso):
δ = 16.329(0.4); 8.435(4.7); 8.403(4.8); 8.385(2.4); 8.354(1.9); 8.253(0.3); 7.716(5.5); 7.690(9.9); 7.671(3.9); 7.655(7.1); 7.631(5.8); 7.605(13.7); 7.601(12.7); 7.594(5.0); 7.589(5.6); 7.578(16.0);
7.567(7.0); 7.563(8.9); 7.540(2.3); 7.517(0.7); 7.409(3.4); 7.402(2.5); 7.383(8.9); 7.364(8.8); 7.360(10.6); 7.357(10.9); 7.349(13.0); 7.330(6.0); 7.303(1.7); 7.294(1.6); 7.272(0.4); 7.204(4.7); 7.196(4.3);
7.177(5.5); 7.170(4.8); 7.159(2.9); 7.155(3.9); 7.147(3.4); 7.139(2.7); 7.115(1.3); 7.109(1.4); 6.990(6.8); 6.964(6.2); 6.879(2.1); 6.857(2.1); 5.756(0.4); 4.568(3.0); 4.560(3.2); 4.529(3.0); 4.273(1.0);
3.419(0.7); 3.410(0.4); 3.380(0.5); 3.315(401.0); 3.256(4.1); 3.246(2.8); 3.223(2.8); 3.213(3.9); 3.123(1.1); 3.079(1.5); 3.041(0.9); 2.732(1.1); 2.727(1.6); 2.512(70.4); 2.506(145.9); 2.500(200.8);
2.494(146.9); 2.488(70.5); 2.311(1.2); 2.276(3.3); 2.270(3.8); 2.236(2.8); 2.225(2.4); 2.193(1.2); 1.997(1.3); 1.969(1.2); 1.934(1.5); 1.892(1.2); 1.829(3.7); 1.791(5.8); 1.758(5.4); 1.729(2.8); 1.698(2.5);
1.674(2.3); 1.663(2.3); 1.632(2.2); 1.601(2.3); 1.555(6.5); 1.525(5.4); 1.462(5.1); 1.431(4.1); 1.397(2.8); 1.297(1.6); 1.275(1.4); 1.265(1.5); 1.234(2.0); 1.183(0.6); 1.107(0.4); 0.859(0.5); 0.833(0.4);
0.011(3.0); −0.001(83.6); −0.011(3.8); −0.029(0.5); −0.045(0.4); −0.199(0.4)
Example 408: ¹H-NMR(299.9 MHz, dmso):
δ = 8.571(0.3); 8.510(3.6); 8.477(4.7); 8.431(5.1); 8.423(5.0); 8.416(4.7); 8.408(4.4); 8.394(2.9); 8.387(3.0); 8.378(3.0); 8.371(2.8); 8.148(0.4); 8.073(0.5); 7.759(0.5); 7.735(0.4); 7.707(0.4); 7.672(0.3);
7.565(5.9); 7.558(6.3); 7.539(4.0); 7.532(4.3); 7.492(4.4); 7.473(11.3); 7.465(16.0); 7.448(12.5); 7.425(7.8); 7.410(5.1); 7.401(10.0); 7.395(7.9); 7.375(1.9); 7.368(1.7); 7.293(8.2); 7.264(5.5); 6.639(0.7);
6.572(0.3); 4.559(2.3); 4.532(2.3); 4.278(1.1); 4.247(1.1); 3.510(0.7); 3.496(0.5); 3.398(0.9); 3.382(1.3); 3.314(492.4); 3.273(3.7); 3.228(3.1); 3.182(0.4); 3.098(1.0); 3.066(1.5); 2.727(2.4); 2.660(0.4);
2.634(0.6); 2.507(253.0); 2.501(327.1); 2.434(1.3); 2.373(0.5); 2.303(0.4); 2.271(2.4); 2.182(0.8); 2.141(1.8); 2.108(2.0); 2.066(1.0); 2.018(1.6); 1.908(0.4); 1.835(4.7); 1.798(6.9); 1.748(3.3); 1.734(3.0);
1.687(1.6); 1.631(1.6); 1.579(3.9); 1.559(3.3); 1.507(3.5); 1.467(5.6); 1.371(1.9); 1.337(3.0); 1.236(7.4); 1.085(0.3); 0.856(0.9); 0.811(0.4); 0.196(0.7); 0.011(7.7); 0.000(141.6); −0.066(0.6); −0.197(0.8)
Example 409: ¹H-NMR(299.9 MHz, dmso):
δ = 8.378(4.5); 8.347(4.5); 8.306(1.5); 8.276(1.4); 8.176(1.1); 8.148(1.1); 8.100(1.1); 8.072(1.3); 7.758(0.7); 7.732(1.0); 7.707(0.8); 7.559(8.4); 7.552(9.5); 7.531(2.8); 7.524(3.2); 7.469(2.0); 7.441(4.3);
7.431(3.7); 7.419(6.2); 7.411(12.0); 7.405(13.4); 7.392(11.2); 7.386(16.0); 7.360(11.5); 7.351(6.8); 7.343(4.0); 7.335(6.9); 7.328(6.7); 7.315(14.4); 7.305(4.5); 7.287(8.4); 7.267(1.3); 7.041(6.2); 7.037(5.8);
7.017(7.7); 7.012(6.7); 6.993(1.8); 6.638(2.9); 4.572(2.9); 4.549(2.9); 4.237(0.6); 3.313(266.9); 3.264(3.9); 3.232(2.8); 3.220(3.9); 3.088(0.9); 2.726(1.3); 2.506(147.8); 2.500(191.7); 2.494(140.4);
2.270(1.7); 2.202(2.5); 2.167(2.4); 2.126(1.0); 1.985(1.0); 1.822(5.0); 1.787(7.7); 1.730(2.9); 1.717(3.6); 1.680(2.7); 1.647(2.3); 1.606(2.6); 1.563(4.6); 1.482(4.8); 1.446(6.1); 1.357(1.4); 1.235(1.6);
0.195(0.5); 0.010(7.2); −0.001(108.6); −0.198(0.5)
Example 410: ¹H-NMR(299.9 MHz, dmso):
δ = 7.920(1.0); 7.880(1.5); 7.847(1.3); 7.538(2.4); 7.531(2.8); 7.526(2.2); 7.519(2.0); 7.407(0.9); 7.379(2.6); 7.356(3.1); 7.349(2.9); 7.328(2.6); 7.321(2.5); 7.259(3.3); 7.231(1.9); 4.534(0.8); 4.511(0.8);
4.225(0.4); 4.192(0.4); 3.314(99.9); 3.255(0.9); 3.220(0.8); 3.211(1.1); 3.151(0.5); 2.727(0.4); 2.578(16.0); 2.532(13.5); 2.513(17.1); 2.507(32.1); 2.501(42.1); 2.495(30.8); 2.489(14.8); 2.270(0.5);
2.264(0.5); 2.233(15.6); 2.209(0.8); 2.153(13.3); 1.928(0.5); 1.895(0.8); 1.840(1.2); 1.808(2.1); 1.765(2.0); 1.725(1.3); 1.714(1.4); 1.684(0.7); 1.659(0.7); 1.609(0.6); 1.560(1.2); 1.496(1.7); 1.453(1.8);
1.415(0.8); 1.311(0.8); 1.237(0.5); 0.000(18.5); −0.011(0.7)
Example 411: ¹H-NMR(299.9 MHz, dmso):
δ = 8.630(5.9); 8.598(5.7); 8.570(4.0); 8.541(3.7); 8.178(1.3); 8.150(1.9); 8.101(1.1); 8.073(1.9); 7.759(1.5); 7.732(1.4); 7.705(1.2); 7.574(1.4); 7.550(13.1); 7.543(12.9); 7.523(8.2); 7.516(8.7); 7.501(2.3);
7.479(4.7); 7.472(3.9); 7.451(8.7); 7.439(4.8); 7.432(9.0); 7.423(6.4); 7.411(7.8); 7.403(13.9); 7.389(3.1); 7.383(4.0); 7.370(8.9); 7.363(8.9); 7.352(5.9); 7.344(6.7); 7.335(4.1); 7.323(10.7); 7.316(10.3);
7.270(16.0); 7.242(7.9); 7.114(12.7); 7.089(15.9); 7.071(9.8); 7.061(10.8); 7.046(11.4); 7.018(6.6); 6.639(4.3); 4.570(3.5); 4.560(3.6); 4.528(3.5); 4.290(1.8); 4.257(1.8); 3.677(0.8); 3.508(1.5); 3.495(1.2);
3.393(1.5); 3.380(2.5); 3.313(1267.2); 3.271(3.7); 3.261(5.0); 3.246(3.9); 3.215(4.7); 3.046(3.1); 2.733(4.3); 2.727(6.2); 2.721(4.9); 2.537(11.3); 2.513(297.4); 2.507(617.1); 2.501(851.0); 2.495(621.1);
2.489(294.0); 2.367(0.9); 2.277(4.7); 2.271(6.6); 2.264(4.9); 2.226(1.1); 2.197(1.3); 2.157(3.1); 2.114(3.0); 2.071(1.4); 1.987(2.6); 1.959(2.3); 1.829(5.7); 1.790(6.3); 1.736(10.3); 1.669(1.9); 1.565(6.5);
1.500(5.5); 1.459(8.7); 1.428(6.2); 1.340(3.1); 1.309(4.0); 1.236(3.8); 1.147(0.8); 0.859(1.9); 0.196(2.6); 0.034(1.1); 0.011(23.7); 0.000(687.5); −0.011(28.5); −0.067(1.3); −0.089(0.8); −0.198(3.0); −0.268
(0.9)
Example 412: ¹H-NMR(299.9 MHz, dmso):
δ = 8.443(4.1); 8.411(4.6); 8.391(4.8); 8.361(4.6); 8.019(0.5); 7.779(0.4); 7.761(0.4); 7.720(4.8); 7.697(11.3); 7.677(12.0); 7.655(4.9); 7.619(8.8); 7.593(8.4); 7.586(7.6); 7.573(10.7); 7.566(12.2);
7.557(14.8); 7.551(12.2); 7.531(1.8); 7.461(5.0); 7.453(7.5); 7.433(8.3); 7.424(16.0); 7.396(10.0); 7.389(8.7); 7.367(3.9); 7.360(4.0); 7.347(10.6); 7.319(6.5); 7.063(5.4); 7.039(5.1); 6.969(5.9); 6.945(5.3);
4.553(2.4); 4.544(2.5); 4.522(2.5); 4.513(2.4); 4.260(1.9); 4.226(2.0); 3.313(206.6); 3.282(2.7); 3.271(3.4); 3.239(2.4); 3.228(3.3); 3.179(0.4); 3.119(1.6); 3.083(2.5); 3.056(1.5); 2.733(0.9); 2.726(1.1);
2.512(58.8); 2.506(112.8); 2.500(148.1); 2.494(107.2); 2.489(51.3); 2.359(0.4); 2.270(1.4); 2.264(1.4); 2.222(1.9); 2.179(2.1); 2.136(0.9); 1.987(2.9); 1.959(2.5); 1.826(5.9); 1.784(6.6); 1.735(7.4);
1.655(1.7); 1.615(1.5); 1.607(1.5); 1.550(4.4); 1.487(4.5); 1.448(7.2); 1.435(7.2); 1.398(4.1); 1.312(4.2); 1.235(2.7); 1.183(0.4); 1.173(0.3); 0.882(0.4); 0.859(0.7); 0.836(0.4); 0.195(0.4); 0.010(4.7);
0.000(91.5); −0.011(4.5); −0.067(0.4); −0.199(0.5); −1.282(0.4)
Example 413: ¹H-NMR(399.8 MHz, dmso):
δ = 8.499(4.9); 8.475(5.1); 8.452(1.6); 8.421(8.7); 8.415(8.9); 8.409(9.3); 8.404(8.6); 8.376(2.4); 8.370(2.6); 8.365(2.6); 8.359(2.6); 7.590(8.0); 7.588(8.4); 7.570(10.5); 7.568(10.6); 7.549(2.7); 7.546(2.6);
7.479(0.4); 7.450(5.1); 7.439(4.6); 7.431(13.3); 7.420(14.9); 7.411(14.1); 7.405(16.0); 7.396(5.2); 7.392(10.7); 7.387(5.2); 7.382(4.5); 7.363(3.8); 7.346(8.4); 7.328(8.2); 7.326(7.7); 7.307(1.6); 7.297(8.1);
7.293(9.1); 7.278(4.9); 7.273(4.5); 7.174(4.7); 7.170(4.5); 7.155(6.4); 7.152(6.1); 7.137(4.6); 7.132(3.7); 7.119(2.1); 7.101(1.1); 7.097(1.1); 5.755(1.1); 5.297(0.9); 4.575(3.1); 4.568(3.2); 4.551(3.2);
4.544(3.0); 4.311(0.6); 4.285(0.6); 3.507(0.4); 3.415(0.4); 3.389(0.5); 3.371(1.1); 3.365(0.8); 3.353(1.4); 3.315(348.3); 3.257(2.5); 3.250(4.1); 3.242(2.6); 3.225(2.8); 3.216(4.5); 3.209(2.5); 3.090(0.7);
3.062(1.2); 3.034(0.8); 3.025(0.6); 2.890(0.7); 2.730(0.6); 2.689(0.4); 2.674(0.9); 2.670(1.2); 2.665(0.9); 2.509(66.4); 2.505(137.5); 2.501(186.6); 2.496(134.3); 2.492(62.7); 2.421(0.3); 2.332(0.9);
2.327(1.3); 2.236(1.0); 2.226(1.1); 2.204(2.6); 2.196(2.6); 2.171(2.9); 2.163(2.7); 2.138(1.2); 2.030(1.1); 2.008(0.9); 1.989(0.8); 1.908(1.5); 1.885(1.0); 1.847(5.0); 1.815(6.9); 1.722(2.3); 1.702(2.4);
1.693(2.4); 1.669(1.6); 1.662(1.6); 1.616(1.9); 1.586(6.3); 1.562(4.2); 1.526(3.3); 1.520(3.3); 1.486(4.2); 1.456(2.9); 1.444(3.5); 1.415(2.4); 1.384(1.0); 1.340(0.9); 1.310(1.3); 1.287(1.4); 1.273(1.4);
1.248(2.5); 1.235(4.7); 1.212(1.3); 1.175(0.5); 1.159(0.5); 1.141(0.6); 0.876(1.0); 0.859(2.7); 0.841(1.3); 0.146(0.8); 0.008(7.1); 0.000(174.3); −0.009(6.3); −0.051(0.4); −0.150(0.7)
Example 414: ¹H-NMR(299.9 MHz, dmso):
δ = 13.811(0.5); 8.761(5.1); 8.748(5.1); 8.724(3.4); 8.711(3.3); 8.657(0.5); 8.565(4.2); 8.534(6.5); 8.506(2.9); 8.074(0.5); 8.019(0.6); 7.767(4.2); 7.752(4.2); 7.741(5.2); 7.725(6.7); 7.706(3.0); 7.696(3.4);
7.681(3.2); 7.581(8.1); 7.573(9.3); 7.559(5.4); 7.552(5.9); 7.517(6.2); 7.491(5.0); 7.457(7.0); 7.450(5.0); 7.430(16.0); 7.422(8.2); 7.408(9.2); 7.402(6.6); 7.380(2.1); 7.373(1.9); 7.306(10.5); 7.278(7.8);
6.639(0.7); 6.572(1.0); 4.548(2.5); 4.539(2.6); 4.516(2.7); 4.265(1.2); 4.236(1.2); 3.509(0.5); 3.450(0.5); 3.409(0.9); 3.390(1.0); 3.380(1.3); 3.314(1000.0); 3.246(3.9); 3.235(4.0); 3.180(1.1); 3.059(2.1);
3.046(1.7); 3.033(1.5); 2.991(1.2); 2.727(3.8); 2.721(2.9); 2.513(182.1); 2.507(366.5); 2.501(496.6); 2.495(364.7); 2.489(176.8); 2.450(1.9); 2.357(0.7); 2.271(4.1); 2.265(2.9); 2.235(0.6); 2.188(1.0);
2.175(1.2); 2.146(2.2); 2.134(2.2); 2.102(2.4); 2.025(2.0); 1.988(1.8); 1.908(0.7); 1.826(5.1); 1.787(5.4); 1.750(6.9); 1.656(1.4); 1.553(5.4); 1.522(4.8); 1.467(5.1); 1.423(4.6); 1.392(2.7); 1.336(2.8);
1.236(3.4); 1.147(0.6); 0.998(0.8); 0.975(1.3); 0.950(0.5); 0.854(0.9); 0.829(0.7); 0.196(1.2); 0.075(0.5); 0.011(12.8); 0.000(270.2); −0.011(11.1); −0.199(0.9)
Example 415: ¹H-NMR(299.9 MHz, dmso):
δ = 8.334(4.3); 8.302(4.6); 8.280(3.8); 8.250(3.5); 8.177(1.4); 8.149(1.5); 8.101(1.4); 8.073(1.6); 7.817(7.0); 7.814(7.0); 7.792(10.5); 7.770(6.3); 7.767(5.9); 7.759(1.4); 7.732(1.4); 7.707(1.0); 7.576(1.4);
7.573(1.4); 7.559(8.4); 7.551(10.0); 7.539(6.8); 7.533(7.2); 7.525(1.5); 7.468(3.4); 7.447(6.5); 7.440(14.6); 7.420(16.0); 7.415(13.7); 7.394(9.9); 7.391(8.7); 7.366(6.8); 7.342(7.0); 7.339(6.8); 7.327(11.9);
7.317(4.8); 7.314(4.2); 7.299(7.4); 7.142(4.1); 7.136(4.4); 7.116(6.4); 7.110(7.8); 7.103(4.1); 7.090(3.8); 7.083(6.4); 7.077(5.4); 7.057(2.8); 7.051(2.6); 6.869(6.6); 6.864(6.6); 6.843(8.3); 6.839(10.1);
6.815(4.7); 6.810(4.3); 6.639(4.0); 6.341(0.9); 4.532(2.7); 4.508(2.6); 4.235(1.4); 4.215(1.4); 3.314(83.6); 3.271(3.2); 3.261(2.2); 3.238(2.3); 3.227(3.4); 3.114(1.2); 3.086(2.0); 3.075(1.8); 3.049(1.2);
2.726(0.5); 2.512(25.3); 2.506(50.1); 2.500(66.9); 2.494(48.4); 2.489(23.0); 2.270(1.1); 2.264(1.1); 2.225(2.0); 2.215(2.2); 2.182(2.1); 2.171(2.0); 2.138(0.8); 2.125(0.7); 2.039(2.1); 1.988(0.6); 1.901(2.3);
1.869(5.0); 1.831(5.0); 1.794(4.8); 1.748(3.0); 1.705(4.4); 1.673(5.0); 1.641(2.2); 1.601(1.6); 1.558(3.2); 1.460(6.5); 1.434(6.0); 1.337(2.9); 1.310(3.2); 1.235(2.3); 1.176(0.4); 0.859(0.4); 0.010(1.9);
0.000(41.9); −0.011(1.8)
Example 416: ¹H-NMR(299.9 MHz, dmso):
δ = 8.144(4.8); 7.803(3.4); 7.507(2.9); 7.492(1.8); 7.485(2.1); 7.391(1.0); 7.362(2.3); 7.327(1.6); 7.320(1.6); 7.299(1.1); 7.292(1.0); 7.271(4.1); 7.265(7.6); 7.232(1.4); 7.199(1.3); 4.523(0.8); 4.498(0.8);
4.227(0.4); 4.194(0.3); 3.748(16.0); 3.677(10.6); 3.316(49.2); 3.243(0.9); 3.232(0.6); 3.210(0.7); 3.199(1.0); 3.099(0.5); 3.062(0.3); 2.514(6.7); 2.508(12.9); 2.502(17.3); 2.496(12.8); 2.490(6.3); 2.291(0.4);
2.255(0.6); 2.244(0.7); 2.212(0.7); 2.201(0.6); 2.145(10.3); 2.080(15.4); 1.872(1.5); 1.831(1.3); 1.759(0.7); 1.685(2.7); 1.602(0.5); 1.544(0.9); 1.492(1.2); 1.456(1.7); 1.337(0.5); 1.299(0.6); 1.236(0.6);
0.011(1.0); 0.001(18.5); −0.010(0.9)
Example 418: ¹H-NMR(400.0 MHz, DMSO):
δ = 8.696(8.2); 8.684(8.2); 8.448(5.6); 8.424(5.7); 7.707(5.6); 7.687(9.1); 7.666(14.1); 7.654(7.3); 7.646(16.0); 7.635(11.6); 7.617(8.2); 7.598(3.7); 7.410(4.9); 7.391(8.1); 7.372(3.6); 7.137(7.4); 7.118(6.8);
4.567(1.3); 4.544(3.0); 4.532(2.7); 4.524(2.9); 4.500(1.0); 4.038(0.9); 4.021(0.9); 3.323(48.8); 3.109(2.5); 3.085(4.8); 3.061(2.5); 2.675(0.5); 2.671(0.7); 2.667(0.5); 2.541(0.3); 2.506(80.5); 2.502(106.3);
2.498(79.4); 2.333(0.5); 2.329(0.7); 2.324(0.5); 1.989(4.3); 1.978(1.6); 1.969(1.4); 1.959(2.2); 1.948(3.7); 1.935(2.6); 1.924(2.9); 1.916(2.8); 1.810(5.0); 1.787(8.4); 1.778(8.9); 1.764(7.3); 1.754(8.4);
1.720(4.8); 1.700(3.4); 1.690(4.0); 1.669(5.8); 1.648(5.0); 1.626(2.1); 1.605(4.1); 1.599(4.3); 1.589(4.7); 1.570(5.0); 1.553(5.2); 1.530(4.3); 1.507(3.3); 1.482(2.0); 1.193(1.1); 1.175(2.1); 1.157(1.1);
0.008(0.8); 0.000(24.0); −0.008(1.0)
Example 419: ¹H-NMR(400.0 MHz, CD3CN):
δ = 7.762(4.3); 7.760(4.3); 7.742(4.5); 7.740(4.5); 7.704(2.8); 7.685(4.2); 7.661(3.6); 7.641(4.0); 7.617(2.2); 7.598(3.6); 7.579(1.7); 7.365(2.2); 7.346(3.6); 7.327(1.6); 7.264(2.1); 7.262(2.2); 7.246(4.7);
7.243(4.7); 7.227(2.7); 7.224(2.7); 7.047(2.5); 7.043(2.6); 7.027(3.9); 7.023(3.8); 7.008(2.0); 7.004(2.0); 6.604(3.6); 6.600(3.6); 6.585(3.4); 6.581(3.3); 6.377(1.2); 6.355(1.2); 4.576(0.5); 4.568(0.6);
4.544(1.5); 4.521(1.5); 4.506(0.6); 4.496(0.6); 3.128(1.2); 3.102(2.2); 3.078(1.1); 2.150(1.1); 2.136(15.8); 2.119(1.2); 2.110(1.3); 2.100(2.0); 2.094(1.5); 2.090(1.3); 2.084(1.5); 2.075(1.4); 2.068(1.2);
1.963(2.5); 1.957(4.5); 1.951(17.5); 1.945(30.3); 1.939(39.6); 1.933(27.5); 1.927(14.3); 1.877(1.5); 1.866(2.3); 1.861(2.4); 1.847(3.4); 1.829(4.3); 1.812(4.0); 1.803(3.8); 1.796(3.9); 1.777(3.2); 1.768(2.3);
1.762(2.4); 1.742(2.4); 1.736(2.6); 1.719(3.9); 1.712(3.8); 1.703(2.8); 1.696(2.4); 1.690(1.8); 1.683(2.3); 1.665(1.9); 1.643(1.8); 1.639(1.8); 1.626(1.7); 1.618(2.1); 1.602(1.7); 1.596(1.7); 1.576(1.0);
1.567(0.9); 1.554(0.5); 1.545(0.4); 1.437(16.0); 0.000(1.7)
Example 420: ¹H-NMR(400.0 MHz, CD3CN):
δ = 7.686(10.6); 7.667(13.1); 7.648(0.6); 7.635(6.0); 7.629(7.3); 7.615(11.7); 7.598(9.0); 7.579(3.9); 7.503(2.3); 7.486(7.9); 7.478(7.8); 7.471(16.0); 7.463(6.5); 7.459(7.8); 7.456(7.5); 7.440(2.2);
7.391(5.3); 7.372(8.7); 7.353(4.0); 6.652(6.9); 6.636(6.7); 6.631(5.8); 6.617(0.4); 6.459(2.6); 6.439(2.6); 4.575(1.7); 4.551(3.7); 4.530(3.4); 4.505(1.3); 3.139(2.7); 3.114(5.2); 3.089(2.6); 2.148(35.4);
2.113(0.5); 2.106(0.5); 2.100(0.4); 2.071(0.9); 2.062(2.2); 2.053(1.9); 2.044(3.2); 2.033(4.2); 2.028(3.6); 2.019(3.6); 2.013(3.2); 2.002(1.6); 1.993(0.6); 1.985(0.9); 1.963(4.1); 1.957(6.6); 1.951(30.2);
1.945(53.4); 1.939(69.9); 1.933(47.5); 1.927(24.3); 1.901(0.4); 1.874(2.5); 1.862(4.2); 1.840(5.7); 1.830(6.8); 1.814(6.2); 1.804(8.4); 1.800(8.4); 1.779(10.1); 1.774(9.7); 1.764(8.3); 1.755(7.4); 1.747(8.5);
1.736(6.1); 1.727(11.6); 1.720(8.7); 1.710(8.7); 1.698(5.5); 1.691(5.5); 1.684(4.3); 1.678(4.4); 1.660(3.1); 1.655(3.3); 1.642(3.9); 1.638(3.7); 1.631(3.6); 1.625(3.9); 1.618(4.4); 1.601(3.6); 1.595(3.6);
1.576(2.1); 1.566(2.0); 1.554(1.1); 1.544(0.8); 1.436(1.2); 1.270(0.6); 0.000(3.2)
Example 421: ¹H-NMR(400.0 MHz, CD3CN):
δ = 7.641(1.4); 7.621(2.1); 7.595(3.6); 7.593(3.8); 7.578(2.0); 7.552(1.0); 7.533(1.7); 7.514(0.8); 7.303(1.1); 7.284(1.8); 7.265(0.8); 7.136(1.9); 7.000(3.9); 6.864(1.9); 6.357(0.6); 6.335(0.6); 4.521(0.7);
4.497(0.7); 4.068(0.4); 4.050(0.4); 3.896(0.5); 3.807(16.0); 3.157(0.5); 3.131(1.0); 3.107(0.5); 2.154(4.3); 2.148(8.4); 2.015(0.4); 2.006(0.4); 1.996(0.5); 1.985(0.9); 1.980(0.7); 1.972(2.6); 1.964(1.9);
1.958(2.5); 1.952(9.9); 1.946(16.6); 1.940(21.7); 1.933(14.7); 1.927(7.5); 1.880(0.5); 1.864(0.8); 1.838(1.2); 1.834(1.1); 1.826(0.9); 1.814(1.8); 1.807(1.4); 1.800(1.2); 1.791(1.8); 1.786(1.4); 1.778(1.7);
1.774(1.7); 1.755(1.7); 1.741(1.4); 1.732(1.3); 1.716(1.6); 1.702(0.9); 1.695(1.6); 1.689(1.2); 1.679(1.0); 1.672(0.8); 1.665(0.8); 1.659(0.9); 1.652(0.7); 1.642(1.1); 1.636(0.9); 1.628(1.1); 1.620(1.1);
1.615(1.0); 1.606(1.4); 1.584(0.5); 1.578(0.5); 1.221(0.5); 1.204(1.0); 1.186(0.5); 0.000(1.1)
Example 422: ¹H-NMR(400.0 MHz, DMSO):
δ = 8.633(3.8); 8.624(3.6); 8.315(0.6); 7.605(2.2); 7.601(2.6); 7.586(4.3); 7.582(5.0); 7.567(2.6); 7.563(2.8); 7.551(1.3); 7.547(1.3); 7.538(1.6); 7.533(2.8); 7.519(2.6); 7.512(3.0); 7.508(1.7); 7.499(1.6);
7.495(1.3); 7.340(11.7); 7.318(16.0); 7.303(3.8); 7.288(4.9); 7.282(4.2); 7.279(4.5); 7.276(4.5); 7.271(7.2); 7.255(3.6); 7.253(4.0); 7.204(15.2); 7.183(11.2); 3.902(1.7); 3.325(304.3); 3.275(0.4); 3.170(0.4);
3.037(1.1); 3.026(2.4); 3.018(3.4); 3.007(3.4); 2.998(2.5); 2.986(1.0); 2.675(1.2); 2.671(1.5); 2.666(1.1); 2.579(0.3); 2.541(1.5); 2.510(107.9); 2.506(195.8); 2.502(244.6); 2.497(177.7); 2.333(1.1);
2.329(1.5); 2.324(1.1); 2.099(1.7); 2.090(1.9); 2.083(2.4); 2.075(3.8); 2.067(2.3); 2.060(2.1); 2.051(1.9); 1.351(2.0); 1.337(3.9); 1.325(3.4); 1.314(3.3); 1.301(2.3); 1.259(0.7); 1.249(2.8); 1.233(4.2);
1.229(3.3); 1.218(2.7); 1.214(3.9); 1.199(1.7); 0.000(5.5)
Example 423: ¹H-NMR(400.0 MHz, DMSO):
δ = 8.718(3.9); 8.707(3.8); 8.315(0.6); 7.661(5.4); 7.641(6.1); 7.453(1.2); 7.450(1.3); 7.434(4.4); 7.432(4.1); 7.417(7.3); 7.414(10.2); 7.407(9.0); 7.394(2.8); 7.388(1.5); 7.379(4.3); 7.373(3.0); 7.362(3.2);
7.359(4.0); 7.356(3.0); 7.353(3.3); 7.349(2.0); 7.343(13.9); 7.337(5.1); 7.326(5.0); 7.321(16.0); 7.315(2.0); 7.200(14.9); 7.184(4.2); 7.179(11.0); 7.133(0.5); 7.111(0.6); 6.699(0.5); 6.678(0.4); 3.902(2.0);
3.365(0.3); 3.323(241.2); 3.285(0.3); 3.011(1.0); 3.000(2.3); 2.992(3.2); 2.980(3.2); 2.972(2.4); 2.961(1.0); 2.675(1.2); 2.670(1.7); 2.666(1.2); 2.541(1.2); 2.510(113.8); 2.506(218.8); 2.501(279.4);
2.497(199.1); 2.492(94.7); 2.333(1.2); 2.328(1.7); 2.323(1.2); 2.103(1.6); 2.094(1.7); 2.088(2.2); 2.079(3.5); 2.071(2.1); 2.064(1.9); 2.056(1.8); 1.311(1.7); 1.297(3.8); 1.285(3.2); 1.274(3.1); 1.261(2.4);
1.245(2.5); 1.230(3.9); 1.226(3.3); 1.215(2.4); 1.210(3.6); 1.196(1.6); 0.000(8.9)
Example 424: ¹H-NMR(400.0 MHz, CD3CN):
δ = 7.669(5.0); 7.663(3.1); 7.657(6.2); 7.653(5.6); 7.647(6.7); 7.636(0.9); 7.589(0.5); 7.553(1.6); 7.540(11.9); 7.527(14.8); 7.517(11.2); 7.492(15.5); 7.486(16.0); 7.477(1.5); 7.474(1.4); 7.303(6.7);
7.298(6.2); 7.282(8.9); 7.277(8.4); 7.164(14.6); 7.143(10.8); 7.018(5.6); 7.011(4.8); 7.008(6.1); 7.002(3.2); 6.996(5.3); 6.705(0.4); 6.539(2.8); 5.448(3.8); 4.068(0.4); 4.050(0.4); 4.036(0.6); 4.011(0.6);
3.387(2.0); 3.376(4.1); 3.368(4.4); 3.365(2.9); 3.357(8.1); 3.349(3.0); 3.346(4.6); 3.338(4.4); 3.327(2.2); 2.518(3.0); 2.499(6.5); 2.477(6.8); 2.468(1.9); 2.464(2.7); 2.459(4.7); 2.166(730.0); 2.121(3.3);
2.114(3.1); 2.108(3.6); 2.101(2.5); 2.095(1.3); 1.971(3.1); 1.965(23.0); 1.958(48.5); 1.953(235.6); 1.946(420.5); 1.940(552.4); 1.934(374.6); 1.928(190.4); 1.915(3.4); 1.781(1.4); 1.775(2.4); 1.769(3.3);
1.763(2.3); 1.756(1.2); 1.457(4.3); 1.441(6.3); 1.438(6.4); 1.434(5.5); 1.421(6.2); 1.418(6.6); 1.415(5.2); 1.399(4.9); 1.309(4.5); 1.297(5.2); 1.292(6.9); 1.280(6.9); 1.275(4.5); 1.263(3.6); 1.221(0.4);
1.204(0.8); 1.186(0.4); 0.146(1.8); 0.008(16.7); 0.000(426.6); −0.009(14.4); −0.150(1.8)
Example 425: ¹H-NMR(400.0 MHz, CD3CN):
δ = 8.348(5.3); 8.343(5.3); 8.336(5.4); 8.331(5.3); 7.501(6.0); 7.497(6.0); 7.487(10.4); 7.482(16.0); 7.478(7.4); 7.303(6.7); 7.291(7.1); 7.284(7.4); 7.272(5.8); 7.265(6.1); 7.260(5.7); 7.175(9.9); 7.154(6.8);
6.576(1.6); 5.447(0.3); 3.413(1.4); 3.401(2.8); 3.394(2.9); 3.390(1.8); 3.382(5.5); 3.375(1.9); 3.371(3.0); 3.363(2.9); 3.352(1.4); 2.525(2.0); 2.506(4.2); 2.484(4.3); 2.465(2.0); 2.142(45.0); 2.114(1.2);
2.107(1.1); 2.101(0.8); 2.095(0.5); 1.964(4.6); 1.958(9.4); 1.952(45.9); 1.946(81.9); 1.940(108.0); 1.934(73.1); 1.927(36.8); 1.774(0.5); 1.768(0.6); 1.762(0.4); 1.472(2.8); 1.455(4.0); 1.452(3.7); 1.449(3.2);
1.436(4.0); 1.433(4.2); 1.430(3.3); 1.414(3.2); 1.315(3.0); 1.303(3.3); 1.298(4.6); 1.286(4.6); 1.281(2.7); 1.269(2.7); 0.146(0.4); 0.008(3.0); 0.000(86.3); −0.009(2.8); −0.150(0.4)
Example 426: ¹H-NMR(399.9 MHz, cdcl3):
δ = 7.747(9.7); 7.727(16.0); 7.709(5.3); 7.691(6.7); 7.671(2.5); 7.522(1.0); 7.488(0.5); 7.470(3.9); 7.451(6.0); 7.433(3.3); 7.424(2.0); 7.413(4.1); 7.402(6.4); 7.391(5.9); 7.380(3.4); 7.370(0.9); 7.349(0.5);
7.294(52.1); 7.283(36.8); 7.263(145.5); 7.233(0.7); 7.213(0.6); 6.999(0.8); 5.651(2.5); 5.634(2.5); 5.060(1.0); 5.040(3.2); 5.022(4.5); 5.003(3.2); 4.983(1.0); 4.437(1.3); 4.420(3.1); 4.401(3.1); 2.712(0.8);
2.696(1.8); 2.681(2.5); 2.672(2.2); 2.662(2.6); 2.650(2.2); 2.646(2.1); 2.631(1.2); 2.628(1.2); 2.483(0.6); 2.468(1.0); 2.452(2.3); 2.437(3.6); 2.428(3.9); 2.412(8.3); 2.397(2.8); 2.390(5.0); 2.381(1.8);
2.369(1.9); 2.359(1.6); 2.338(0.6); 2.208(1.5); 2.191(2.4); 2.178(2.0); 2.164(2.8); 2.148(1.5); 2.142(1.6); 2.136(1.8); 2.118(0.9); 1.624(0.7); 1.571(111.2); 1.541(3.2); 1.521(0.7); 1.425(1.0); 1.333(1.6);
1.285(2.3); 1.253(4.9); 1.233(0.5); 0.880(0.8); 0.862(0.4); 0.146(1.0); 0.051(0.8); 0.043(0.5); 0.008(11.3); 0.000(208.4); −0.008(13.3); −0.029(0.6); −0.051(0.8); −0.149(0.9)
Example 427: ¹H-NMR(399.9 MHz, cdcl3):
δ = 7.851(0.6); 7.843(0.6); 7.831(0.6); 7.823(0.8); 7.799(0.6); 7.761(10.0); 7.758(10.4); 7.753(2.8); 7.748(2.8); 7.745(2.8); 7.739(12.2); 7.738(11.8); 7.711(9.6); 7.690(12.2); 7.665(13.6); 7.633(6.8);
7.615(9.9); 7.596(4.3); 7.522(1.1); 7.463(0.4); 7.461(0.3); 7.451(0.4); 7.443(0.5); 7.438(0.4); 7.436(0.4); 7.431(0.5); 7.421(0.6); 7.406(5.8); 7.388(9.4); 7.368(4.4); 7.358(1.7); 7.356(1.4); 7.348(1.6);
7.341(0.6); 7.338(0.6); 7.333(0.8); 7.331(0.8); 7.329(0.6); 7.326(0.4); 7.323(0.5); 7.321(0.6); 7.315(0.9); 7.313(1.2); 7.311(1.0); 7.308(0.7); 7.306(0.8); 7.303(0.9); 7.301(0.8); 7.298(0.9); 7.296(1.2);
7.293(1.2); 7.291(1.2); 7.288(1.5); 7.286(1.8); 7.283(1.9); 7.280(2.2); 7.278(3.1); 7.276(4.0); 7.270(8.1); 7.263(180.6); 7.252(4.5); 7.250(3.6); 7.247(3.3); 7.242(14.2); 7.240(12.6); 7.234(2.2); 7.232(1.9);
7.223(7.7); 7.221(7.1); 7.214(1.2); 7.212(1.6); 7.207(0.9); 7.205(0.7); 7.202(0.4); 7.199(0.5); 7.197(0.5); 7.195(0.4); 7.192(0.4); 7.189(0.4); 7.187(0.4); 7.179(0.3); 7.169(0.3); 7.162(0.4); 7.124(0.3);
7.122(0.4); 7.119(0.5); 7.103(0.7); 7.099(0.7); 7.094(0.4); 7.090(0.5); 7.084(0.6); 7.080(0.6); 7.072(0.5); 7.064(0.3); 7.060(0.4); 7.054(0.3); 7.049(0.4); 7.047(0.4); 7.039(0.6); 7.021(11.2); 7.017(15.1);
7.013(11.9); 7.004(13.5); 6.999(16.0); 6.994(10.7); 6.986(7.1); 6.982(4.5); 5.445(3.9); 5.428(3.9); 5.304(8.7); 5.052(1.4); 5.033(4.4); 5.014(6.0); 4.996(4.4); 4.977(1.4); 4.429(2.0); 4.410(5.0); 4.392(4.8);
4.372(1.8); 4.131(0.8); 4.113(0.9); 4.095(0.4); 2.718(1.4); 2.715(1.5); 2.700(3.2); 2.697(3.1); 2.685(3.8); 2.682(3.8); 2.679(3.8); 2.674(3.7); 2.670(3.7); 2.666(3.9); 2.664(3.8); 2.652(3.9); 2.649(3.6);
2.633(2.0); 2.630(1.8); 2.512(1.6); 2.497(2.5); 2.482(4.1); 2.467(4.7); 2.457(4.5); 2.442(4.9); 2.426(2.8); 2.419(2.4); 2.416(2.3); 2.398(5.7); 2.389(2.0); 2.385(2.2); 2.380(5.1); 2.377(5.9); 2.368(3.4);
2.359(2.9); 2.356(2.8); 2.350(2.8); 2.347(3.0); 2.328(1.5); 2.325(1.4); 2.303(0.4); 2.267(3.1); 2.250(4.5); 2.241(3.2); 2.235(4.2); 2.222(4.5); 2.210(3.3); 2.204(2.4); 2.195(2.9); 2.179(1.6); 2.049(3.4);
1.991(0.3); 1.803(0.4); 1.673(0.4); 1.671(0.4); 1.665(0.4); 1.663(0.4); 1.658(0.4); 1.655(0.4); 1.653(0.5); 1.648(0.6); 1.645(0.6); 1.643(0.5); 1.638(0.7); 1.635(0.7); 1.630(0.9); 1.628(0.9); 1.620(1.6);
1.618(1.3); 1.616(1.2); 1.612(1.3); 1.610(1.5); 1.608(1.6); 1.603(1.8); 1.600(2.0); 1.598(2.2); 1.595(2.5); 1.593(2.9); 1.590(3.5); 1.570(192.7); 1.557(2.6); 1.554(1.8); 1.549(1.0); 1.547(0.8); 1.544(0.5);
1.541(0.4); 1.539(0.5); 1.537(0.3); 1.519(1.0); 1.514(0.5); 1.512(0.3); 1.295(0.4); 1.279(1.3); 1.261(2.7); 1.253(3.2); 1.243(1.2); 0.880(0.4); 0.152(0.4); 0.145(1.4); 0.140(0.3); 0.125(0.3); 0.122(0.3);
0.120(0.3); 0.115(0.4); 0.112(0.3); 0.109(0.3); 0.107(0.4); 0.105(0.5); 0.102(0.4); 0.099(0.5); 0.097(0.5); 0.095(0.5); 0.092(0.4); 0.089(0.5); 0.087(0.6); 0.085(0.5); 0.082(0.6); 0.080(0.7); 0.077(0.7);
0.075(0.5); 0.072(0.6); 0.070(0.8); 0.067(0.7); 0.064(0.7); 0.062(0.9); 0.060(1.0); 0.057(1.0); 0.054(1.1); 0.052(1.5); 0.050(1.8); 0.045(1.4); 0.042(1.6)
Example 429: ¹H-NMR(399.9 MHz, cdcl3):
δ = 7.771(0.5); 7.742(9.8); 7.720(16.0); 7.707(8.5); 7.689(8.5); 7.670(3.2); 7.651(0.6); 7.522(0.4); 7.471(4.5); 7.452(7.3); 7.433(3.5); 7.410(0.4); 7.403(0.4); 7.396(0.6); 7.375(0.5); 7.325(0.5); 7.320(0.4);
7.315(0.6); 7.310(0.5); 7.302(0.7); 7.300(0.7); 7.298(0.7); 7.292(0.9); 7.290(1.0); 7.272(13.3); 7.263(49.8); 7.251(28.7); 7.224(29.1); 7.203(11.7); 7.174(0.3); 6.999(0.3); 5.584(3.6); 5.566(3.7); 5.041(1.3);
5.022(4.2); 5.003(5.9); 4.984(4.1); 4.964(1.2); 4.433(1.8); 4.414(4.2); 4.397(4.0); 4.378(1.6); 2.708(1.1); 2.691(2.6); 2.676(3.4); 2.667(2.9); 2.658(3.5); 2.645(2.9); 2.625(1.4); 2.480(0.8); 2.466(1.4);
2.450(3.3); 2.434(6.5); 2.424(5.1); 2.411(10.7); 2.392(7.4); 2.381(2.3); 2.371(2.5); 2.361(1.9); 2.340(0.8); 2.204(2.0); 2.186(3.3); 2.176(2.7); 2.168(2.6); 2.159(3.8); 2.143(2.0); 2.132(2.4); 2.113(1.2);
1.634(0.8); 1.583(68.6); 1.533(0.4); 1.253(1.3); 0.146(0.4); 0.051(0.7); 0.049(0.6); 0.043(0.4); 0.041(0.5); 0.039(0.5); 0.033(0.6); 0.031(0.7); 0.029(0.7); 0.023(1.0); 0.021(1.2); 0.016(1.8); 0.013(2.2);
0.000(71.2); −0.020(0.4); −0.050(0.4); −0.150(0.3)
Example 430: ¹H-NMR(400.0 MHz, DMSO):
δ = 8.649(4.6); 8.627(4.6); 7.855(0.4); 7.730(0.4); 7.710(5.1); 7.691(8.8); 7.654(11.7); 7.635(16.0); 7.615(3.0); 7.490(0.4); 7.469(0.3); 7.442(2.1); 7.433(4.6); 7.426(4.4); 7.420(4.6); 7.414(7.3); 7.404(7.3);
7.399(3.6); 7.395(3.6); 7.388(3.8); 7.383(4.0); 7.367(1.8); 7.154(0.5); 7.134(0.5); 7.114(0.3); 7.051(1.9); 7.043(11.2); 7.024(14.4); 7.004(9.5); 6.996(1.6); 4.909(0.6); 4.889(2.4); 4.869(3.6); 4.851(2.5);
4.831(0.8); 4.164(1.2); 4.145(3.3); 4.126(3.1); 3.320(168.2); 2.675(2.7); 2.670(3.7); 2.666(2.7); 2.540(8.3); 2.523(9.0); 2.510(223.9); 2.506(453.2); 2.501(593.2); 2.496(423.5); 2.492(201.8); 2.463(2.9);
2.456(3.9); 2.444(3.7); 2.431(3.2); 2.406(3.2); 2.381(2.7); 2.364(1.4); 2.332(2.9); 2.328(3.9); 2.323(2.9); 2.263(1.1); 2.243(2.6); 2.236(2.1); 2.227(2.6); 2.222(3.3); 2.216(2.7); 2.206(1.6); 2.199(2.2);
2.194(2.2); 2.177(1.0); 2.129(1.7); 2.114(2.4); 2.105(2.3); 2.090(3.3); 2.076(2.4); 2.063(2.2); 2.051(0.8); 2.037(0.4); 2.027(0.5); 2.007(0.6); 1.989(0.5); 1.977(0.4); 1.240(1.8); 0.854(0.6); 0.146(0.5);
0.008(3.7); 0.000(122.6); −0.009(4.4); −0.150(0.5)
Example 432: ¹H-NMR(399.9 MHz, cdcl3):
δ = 7.794(0.6); 7.778(0.9); 7.710(12.9); 7.691(16.0); 7.640(5.7); 7.622(8.2); 7.603(4.4); 7.547(6.4); 7.528(7.1); 7.414(5.6); 7.397(7.9); 7.378(4.6); 7.344(2.1); 7.262(63.4); 7.239(18.0); 7.091(0.3);
7.000(0.4); 6.058(4.1); 5.065(4.0); 5.048(5.5); 5.029(3.9); 4.958(0.4); 4.413(5.0); 4.396(4.8); 2.924(0.3); 2.682(4.2); 2.668(4.1); 2.654(3.5); 2.635(1.6); 2.462(4.1); 2.448(5.1); 2.436(5.3); 2.423(6.3);
2.405(6.5); 2.383(5.5); 2.333(1.4); 2.306(0.7); 2.233(2.3); 2.214(3.9); 2.190(4.7); 2.069(0.3); 2.060(0.3); 1.994(0.4); 1.969(0.5); 1.947(0.4); 1.878(0.4); 1.847(0.4); 1.813(0.4); 1.753(0.5); 1.728(0.4);
1.566(56.6); 1.344(0.5); 1.255(2.4); 0.146(0.6); −0.001(96.2); −0.149(0.8)
Example 434: ¹H-NMR(400.0 MHz, DMSO):
δ = 8.904(0.4); 8.883(0.4); 8.510(5.3); 8.489(5.4); 8.308(4.0); 7.865(0.5); 7.846(0.6); 7.778(5.9); 7.759(10.8); 7.724(5.6); 7.705(8.7); 7.685(12.2); 7.664(13.9); 7.655(7.6); 7.621(0.6); 7.588(1.7);
7.569(11.3); 7.560(16.0); 7.551(10.0); 7.532(1.4); 7.475(4.8); 7.456(8.2); 7.437(4.2); 7.413(0.8); 6.788(0.3); 6.769(6.0); 6.762(5.9); 6.748(6.1); 4.848(1.3); 4.834(3.0); 4.828(3.2); 4.815(3.2); 4.806(3.0);
4.785(1.2); 4.197(1.4); 4.179(3.7); 4.161(3.7); 4.141(1.3); 3.898(2.8); 3.730(0.3); 3.390(0.5); 3.323(545.5); 3.247(0.6); 3.217(0.5); 3.169(0.4); 2.676(2.4); 2.672(3.4); 2.667(2.5); 2.542(2.8); 2.511(229.2);
2.507(438.9); 2.503(560.0); 2.498(412.2); 2.473(6.1); 2.462(4.7); 2.454(2.8); 2.443(2.1); 2.334(2.7); 2.329(3.5); 2.325(2.6); 2.295(0.5); 2.266(0.8); 2.244(2.6); 2.223(3.7); 2.216(3.9); 2.211(3.3); 2.197(2.9);
2.181(1.1); 2.173(1.5); 2.157(0.7); 2.136(0.6); 2.111(2.3); 2.088(3.1); 2.080(4.0); 2.068(3.5); 2.043(1.6); 2.022(0.6); 1.998(0.5); 1.967(0.4); 1.941(0.3); 1.892(0.3); 1.871(0.4); 1.858(0.4); 1.728(0.3);
1.700(0.4); 1.680(0.4); 1.625(0.3); 1.601(0.4); 1.477(0.4); 1.453(0.5); 1.401(0.3); 1.337(0.4); 1.298(0.7); 1.260(0.7); 1.236(0.8); 0.000(19.6); −0.008(0.8)
Example 436: ¹H-NMR(399.9 MHz, cdcl3):
δ = 7.758(7.3); 7.739(9.7); 7.723(16.0); 7.705(6.9); 7.686(2.1); 7.588(0.4); 7.569(0.3); 7.522(0.8); 7.488(3.3); 7.469(5.3); 7.452(2.7); 7.449(2.5); 7.391(0.5); 7.382(4.1); 7.379(4.8); 7.377(4.9); 7.374(4.6);
7.362(5.3); 7.359(5.8); 7.357(6.4); 7.354(6.0); 7.346(0.7); 7.344(0.6); 7.342(0.4); 7.336(0.4); 7.334(0.5); 7.332(0.4); 7.329(0.5); 7.326(0.6); 7.324(0.4); 7.321(0.4); 7.319(0.5); 7.316(0.6); 7.314(0.8);
7.311(0.6); 7.309(0.6); 7.307(0.5); 7.304(0.4); 7.301(0.6); 7.299(0.7); 7.297(0.6); 7.294(0.6); 7.291(0.9); 7.289(0.9); 7.286(0.8); 7.284(1.1); 7.281(1.4); 7.279(1.5); 7.276(1.7); 7.274(2.8); 7.271(3.9);
7.263(119.5); 7.258(5.7); 7.255(3.6); 7.253(2.1); 7.250(1.3); 7.247(1.4); 7.245(1.5); 7.240(7.1); 7.235(1.8); 7.230(8.2); 7.226(14.4); 7.221(21.4); 7.213(1.5); 7.210(1.1); 7.208(0.9); 7.201(7.8); 7.195(0.6);
7.193(0.5); 7.190(0.3); 7.145(6.8); 7.142(9.7); 7.139(6.1); 7.132(1.0); 7.125(4.3); 7.123(6.1); 7.113(0.4); 6.999(0.7); 5.586(2.6); 5.568(2.7); 5.047(1.1); 5.027(3.4); 5.008(4.8); 4.989(3.4); 4.970(1.0);
4.443(1.3); 4.425(3.3); 4.407(3.2); 4.388(1.2); 2.715(0.9); 2.711(1.0); 2.701(1.2); 2.695(2.0); 2.681(2.8); 2.675(2.5); 2.671(2.5); 2.666(2.2); 2.661(2.8); 2.649(2.4); 2.645(2.2); 2.640(1.4); 2.630(1.4);
2.626(1.2); 2.488(0.7); 2.473(1.3); 2.457(2.7); 2.442(4.2); 2.432(4.1); 2.417(10.0); 2.402(2.9); 2.396(5.8); 2.386(1.8); 2.375(2.0); 2.365(1.7); 2.345(0.7); 2.202(1.7); 2.184(2.9); 2.177(2.2); 2.173(2.0);
2.167(1.9); 2.165(2.0); 2.156(3.0); 2.154(2.9); 2.148(1.8); 2.142(1.5); 2.140(1.6); 2.136(1.6); 2.134(1.7); 2.129(2.1); 2.111(1.0); 1.650(0.3); 1.644(0.4); 1.642(0.4); 1.640(0.4); 1.637(0.3); 1.634(0.4);
1.632(0.5); 1.630(0.5); 1.627(0.6); 1.624(0.8); 1.622(1.3); 1.617(0.7); 1.615(0.8); 1.612(0.7); 1.609(0.7); 1.607(0.9); 1.605(0.9); 1.602(0.8); 1.599(1.1); 1.597(1.3); 1.595(1.4); 1.592(1.4); 1.589(2.0);
1.587(2.4); 1.585(2.6); 1.582(3.9); 1.579(6.1); 1.577(9.8); 1.572(161.7); 1.563(4.5); 1.561(2.9); 1.559(1.5); 1.556(1.1); 1.553(1.1); 1.551(0.7); 1.546(0.5); 1.543(0.5); 1.521(0.7); 1.518(0.5); 1.516(0.3);
1.253(1.6); 0.146(1.0); 0.108(0.3); 0.103(0.3); 0.100(0.4); 0.098(0.4); 0.093(0.4); 0.090(0.4); 0.083(0.4); 0.081(0.4); 0.075(0.4); 0.073(0.5); 0.071(0.4); 0.068(0.4); 0.065(0.6); 0.063(0.6); 0.061(0.4);
0.058(0.7); 0.055(0.9); 0.053(0.9); 0.050(1.3); 0.048(1.0); 0.046(0.9); 0.043(0.8); 0.040(0.9); 0.038(1.2); 0.036(1.1); 0.033(0.9); 0.030(1.4); 0.028(1.6); 0.026(1.4); 0.023(1.7); 0.020(2.2); 0.018(2.5);
0.015(2.4); 0.013(3.9); 0.010(5.8); 0.008(10.8); 0.000(189.8); −0.006(8.4); −0.008(8.1); −0.051(0.9); −0.053(0.3); −0.150(0.6)
Example 437: ¹H-NMR(400.0 MHz, DMSO):
δ = 8.961(0.5); 8.872(9.9); 8.869(9.9); 8.690(4.6); 8.668(4.7); 8.619(0.4); 8.582(0.6); 8.450(10.1); 8.447(9.8); 8.315(0.9); 7.882(0.4); 7.862(0.4); 7.739(4.9); 7.719(7.1); 7.677(0.5); 7.652(11.8); 7.633(16.0);
7.614(3.1); 7.461(0.3); 7.426(3.9); 7.407(6.2); 7.388(2.7); 4.898(2.0); 4.878(3.2); 4.861(2.3); 4.843(0.9); 4.214(1.2); 4.195(3.1); 4.175(3.0); 3.902(3.0); 3.389(0.4); 3.323(259.4); 2.675(2.2); 2.671(2.9);
2.666(2.2); 2.637(0.4); 2.621(0.4); 2.569(1.5); 2.551(2.7); 2.541(3.7); 2.510(195.5); 2.506(366.4); 2.502(463.3); 2.497(336.2); 2.493(168.2); 2.471(4.4); 2.444(2.6); 2.425(1.1); 2.403(0.3); 2.333(2.1);
2.328(2.7); 2.324(2.1); 2.297(0.4); 2.285(0.7); 2.280(0.9); 2.262(2.2); 2.248(2.6); 2.242(3.0); 2.226(1.6); 2.220(2.2); 2.215(2.0); 2.198(0.9); 2.194(0.9); 2.138(1.5); 2.124(2.0); 2.116(2.1); 2.099(2.8);
2.086(2.3); 2.074(2.2); 2.050(0.6); 1.901(0.3); 1.236(0.6); 0.000(17.5)
Example 438: ¹H-NMR(400.0 MHz, DMSO):
δ = 8.693(4.3); 8.673(4.4); 8.445(9.1); 8.441(9.2); 8.433(9.6); 8.430(9.1); 8.315(0.9); 7.897(9.2); 7.894(9.0); 7.876(10.3); 7.873(9.5); 7.456(9.4); 7.445(9.0); 7.436(8.5); 7.424(8.3); 7.282(1.4); 7.266(3.1);
7.261(3.0); 7.245(6.2); 7.229(3.2); 7.225(3.8); 7.208(1.8); 7.002(1.0); 6.998(1.3); 6.986(8.9); 6.965(16.0); 6.944(7.5); 6.932(1.1); 4.885(0.8); 4.865(2.6); 4.848(3.6); 4.830(2.5); 4.814(0.8); 4.215(1.4);
4.195(4.9); 4.174(5.2); 4.154(2.0); 3.902(2.3); 3.403(0.4); 3.393(0.5); 3.328(648.9); 3.293(1.3); 3.281(0.8); 3.266(0.8); 3.255(0.4); 3.167(1.3); 2.676(1.8); 2.671(2.4); 2.667(1.7); 2.604(0.9); 2.577(2.4);
2.561(3.5); 2.541(5.0); 2.511(165.8); 2.507(316.7); 2.502(403.5); 2.498(291.6); 2.493(143.0); 2.458(3.4); 2.449(2.6); 2.440(2.9); 2.415(1.6); 2.376(1.4); 2.353(2.1); 2.333(4.4); 2.329(4.2); 2.320(5.4);
2.301(4.4); 2.285(4.0); 2.272(2.1); 2.260(1.8); 2.244(0.9); 0.000(5.8)
Example 439: ¹H-NMR(400.0 MHz, DMSO):
δ = 8.902(4.9); 8.883(4.8); 8.315(0.8); 7.491(0.4); 7.469(0.4); 7.448(1.6); 7.432(3.4); 7.427(3.6); 7.411(6.6); 7.394(3.9); 7.390(4.3); 7.373(1.9); 7.309(1.5); 7.293(3.2); 7.289(3.3); 7.272(6.1); 7.256(3.6);
7.251(3.8); 7.235(1.9); 7.215(0.3); 7.165(0.6); 7.147(0.9); 7.130(0.7); 7.110(0.6); 7.088(0.6); 7.077(0.6); 7.058(2.8); 7.051(10.9); 7.031(15.0); 7.011(9.7); 7.003(3.2); 6.990(9.0); 6.969(16.0); 6.948(7.6);
6.936(1.1); 4.861(0.9); 4.843(2.9); 4.826(4.1); 4.808(3.1); 4.791(0.9); 4.179(2.0); 4.158(5.2); 4.138(5.2); 4.118(2.0); 3.902(2.8); 3.385(0.5); 3.372(0.6); 3.323(297.9); 3.244(0.4); 3.168(1.1); 2.670(2.9);
2.650(0.5); 2.612(1.2); 2.586(2.3); 2.568(4.3); 2.541(4.9); 2.506(365.6); 2.501(465.4); 2.497(348.8); 2.455(3.4); 2.427(3.4); 2.417(2.4); 2.409(3.0); 2.390(1.5); 2.358(1.5); 2.332(4.3); 2.328(4.7); 2.323(4.7);
2.296(2.3); 2.273(1.1); 2.246(1.9); 2.232(2.8); 2.219(3.3); 2.205(3.9); 2.193(2.7); 2.179(2.0); 2.165(1.0); 1.259(0.3); 1.236(0.4); 0.793(0.5); 0.774(0.9); 0.755(0.5); 0.000(13.2)
Example 440: ¹H-NMR(400.0 MHz, DMSO):
δ = 8.606(5.2); 8.587(5.2); 8.371(0.4); 8.350(0.4); 8.315(1.1); 7.792(9.2); 7.790(9.4); 7.773(9.9); 7.771(9.7); 7.466(0.4); 7.446(0.6); 7.428(0.4); 7.332(1.7); 7.324(4.9); 7.321(5.1); 7.316(4.0); 7.311(4.2);
7.305(10.9); 7.302(10.9); 7.295(6.9); 7.286(7.1); 7.284(6.7); 7.279(4.0); 7.274(4.5); 7.258(2.1); 7.226(0.5); 7.211(0.6); 7.169(0.4); 7.150(0.4); 7.144(0.4); 7.132(0.6); 7.101(5.2); 7.097(5.2); 7.082(8.3);
7.078(8.2); 7.063(4.6); 7.058(4.9); 7.040(9.1); 7.019(16.0); 6.998(7.6); 6.986(1.1); 6.697(8.4); 6.694(8.3); 6.678(8.2); 6.675(7.6); 6.566(0.5); 6.563(0.5); 6.547(0.4); 6.544(0.5); 4.816(0.9); 4.798(2.7);
4.779(3.8); 4.760(2.8); 4.742(0.9); 4.189(2.1); 4.169(5.4); 4.149(5.9); 4.129(2.2); 4.094(0.4); 4.073(0.3); 3.902(4.4); 3.438(0.5); 3.418(0.5); 3.400(0.8); 3.389(0.9); 3.378(1.2); 3.326(759.3); 3.267(0.6);
3.259(0.6); 3.245(0.4); 3.169(0.8); 2.675(2.6); 2.671(3.4); 2.666(2.6); 2.640(1.0); 2.616(2.9); 2.599(3.7); 2.584(3.0); 2.572(2.1); 2.541(2.9); 2.510(242.1); 2.506(454.7); 2.502(570.8); 2.497(409.1);
2.493(198.5); 2.436(2.4); 2.424(3.1); 2.407(3.0); 2.384(1.8); 2.357(1.5); 2.333(5.3); 2.328(5.9); 2.324(5.6); 2.318(5.6); 2.299(6.7); 2.285(4.9); 2.272(2.4); 2.260(1.9); 2.246(0.8); 2.227(0.4); 2.211(0.4);
1.237(0.5); 0.799(0.4); 0.782(0.4); 0.000(8.2)
Example 441: ¹H-NMR(601.6 MHz, DMSO):
δ = 8.705(6.1); 8.693(6.2); 8.471(0.4); 7.677(0.7); 7.638(0.5); 7.575(15.7); 7.572(16.0); 7.482(0.5); 7.425(0.7); 7.403(7.6); 7.400(7.5); 7.390(8.1); 7.387(7.7); 7.324(1.4); 7.311(3.5); 7.300(5.7); 7.288(3.9);
7.275(1.9); 7.237(0.6); 7.205(0.6); 7.108(0.5); 7.030(8.7); 7.016(15.6); 7.003(7.9); 6.926(12.2); 6.912(11.7); 6.810(0.7); 6.796(0.6); 4.813(3.5); 4.802(4.9); 4.790(3.5); 4.194(2.0); 4.181(5.6); 4.168(5.6);
4.154(2.1); 4.097(0.7); 3.914(0.4); 3.330(43.0); 3.185(2.2); 3.176(2.2); 2.625(1.0); 2.580(4.2); 2.567(3.2); 2.552(2.1); 2.513(157.6); 2.433(3.7); 2.419(3.2); 2.405(1.6); 2.361(1.4); 2.335(3.5); 2.319(3.0);
2.303(3.4); 2.293(2.8); 2.285(3.3); 2.276(4.1); 2.267(2.8); 2.258(2.1); 1.246(0.4); 0.791(0.8)
Example 442: ¹H-NMR(399.9 MHz, cdcl3):
δ = 7.728(0.4); 7.603(0.7); 7.521(1.4); 7.468(12.1); 7.411(5.9); 7.391(8.0); 7.367(6.0); 7.347(9.4); 7.314(4.6); 7.312(4.1); 7.300(9.4); 7.280(13.1); 7.262(202.1); 7.211(4.4); 7.194(3.3); 7.190(3.4);
7.173(5.9); 7.153(3.9); 7.136(1.9); 7.105(0.3); 7.081(0.4); 7.057(0.4); 6.998(1.1); 6.937(0.5); 6.917(1.8); 6.907(9.1); 6.887(16.0); 6.866(7.8); 6.343(2.7); 6.326(2.8); 5.127(1.1); 5.107(3.2); 5.086(4.0);
5.069(3.2); 5.048(1.1); 4.437(2.0); 4.416(4.3); 4.396(4.3); 4.374(1.9); 3.476(0.3); 2.924(0.7); 2.716(0.8); 2.693(1.8); 2.677(2.5); 2.649(2.5); 2.628(1.3); 2.597(1.2); 2.580(1.5); 2.567(2.4); 2.551(3.3);
2.537(2.9); 2.523(2.9); 2.508(1.5); 2.484(1.5); 2.461(2.9); 2.443(3.3); 2.413(2.3); 2.389(1.4); 2.375(2.2); 2.359(2.7); 2.345(2.9); 2.331(3.6); 2.316(2.2); 2.302(1.9); 2.286(0.9); 1.847(0.4); 1.668(0.8);
1.617(5.7); 1.565(297.5); 1.516(4.8); 1.514(4.8); 1.481(0.6); 1.466(0.6); 1.372(0.5); 1.333(0.5); 1.286(1.3); 1.254(6.6); 0.906(0.6); 0.888(1.1); 0.881(1.0); 0.145(1.4); 0.051(5.1); 0.049(4.3); 0.001(240.7); −0.001
(268.2); −0.050(4.7); −0.052(4.2); −0.149(1.4)
Example 443: ¹H-NMR(400.0 MHz, DMSO):
δ = 8.643(4.4); 8.625(4.4); 8.315(0.8); 7.481(0.3); 7.462(0.5); 7.442(0.5); 7.424(0.5); 7.384(3.3); 7.364(16.0); 7.350(7.3); 7.346(6.9); 7.330(3.4); 7.326(2.8); 7.314(3.1); 7.310(3.1); 7.294(5.6); 7.278(7.3);
7.273(7.3); 7.259(7.2); 7.242(3.4); 7.238(3.2); 7.215(0.5); 7.199(0.3); 7.071(0.5); 7.043(1.5); 7.031(8.2); 7.010(14.2); 6.989(6.7); 6.977(1.0); 6.869(7.5); 6.851(6.7); 6.848(6.2); 4.835(0.9); 4.819(2.6);
4.801(3.7); 4.781(2.6); 4.765(0.8); 4.193(1.8); 4.173(4.6); 4.153(5.3); 4.134(1.9); 3.902(2.5); 3.323(246.0); 3.279(0.6); 2.674(1.9); 2.670(2.4); 2.666(1.9); 2.619(1.2); 2.594(2.7); 2.576(3.4); 2.561(3.3);
2.540(3.5); 2.506(319.8); 2.501(397.2); 2.497(293.3); 2.440(2.7); 2.421(2.8); 2.401(2.8); 2.382(1.8); 2.358(1.5); 2.328(5.1); 2.323(4.7); 2.314(4.5); 2.297(6.0); 2.282(4.5); 2.267(2.1); 2.256(1.7); 2.243(0.8);
1.239(0.3); 0.000(11.9)
Example 444: ¹H-NMR(399.9 MHz, cdcl3):
δ = 7.700(0.7); 7.679(0.8); 7.567(0.5); 7.546(0.7); 7.522(1.0); 7.458(11.2); 7.454(5.2); 7.441(5.5); 7.437(16.0); 7.415(1.0); 7.394(0.8); 7.383(0.5); 7.369(1.0); 7.348(1.0); 7.330(16.0); 7.325(6.2);
7.308(12.0); 7.294(2.1); 7.263(163.2); 7.236(0.6); 7.212(0.5); 7.193(1.3); 7.177(2.3); 7.172(2.4); 7.156(4.5); 7.152(2.1); 7.140(2.3); 7.136(2.9); 7.120(1.4); 6.999(0.9); 6.901(1.3); 6.891(6.2); 6.881(1.8);
6.870(11.2); 6.849(5.4); 6.839(1.3); 6.353(1.8); 6.336(1.9); 5.129(0.7); 5.108(2.1); 5.088(2.6); 5.071(2.1); 5.050(0.7); 4.434(1.2); 4.412(2.8); 4.392(2.8); 4.371(1.3); 2.691(1.2); 2.669(1.7); 2.650(1.6);
2.625(0.8); 2.587(0.7); 2.570(1.0); 2.558(1.6); 2.542(2.2); 2.529(1.9); 2.514(1.9); 2.499(1.0); 2.481(1.0); 2.456(1.8); 2.439(2.2); 2.410(1.4); 2.386(1.0); 2.370(1.5); 2.354(2.1); 2.342(2.0); 2.326(2.4);
2.311(1.5); 2.298(1.2); 2.281(0.6); 2.177(0.7); 1.848(0.4); 1.675(0.6); 1.612(0.7); 1.571(251.8); 1.515(0.7); 1.283(0.6); 1.253(4.0); 0.904(0.6); 0.885(1.1); 0.867(0.6); 0.146(0.9); 0.041(0.4); 0.007(11.6); −0.001
(221.2); −0.045(0.4); −0.056(0.5); −0.150(1.0)
Example 445: ¹H-NMR(399.9 MHz, cdcl3):
δ = 7.748(16.0); 7.631(8.9); 7.612(10.2); 7.558(8.8); 7.540(10.2); 7.522(1.3); 7.477(6.5); 7.457(9.5); 7.423(0.7); 7.385(10.3); 7.365(14.8); 7.344(12.3); 7.321(12.2); 7.305(6.5); 7.286(11.9); 7.263(116.5);
7.221(0.8); 7.208(1.3); 7.189(6.7); 7.186(6.5); 7.169(9.2); 7.151(4.0); 6.998(0.7); 6.426(4.2); 6.407(4.2); 5.303(0.5); 4.876(1.5); 4.854(4.9); 4.833(7.0); 4.811(5.0); 4.789(1.5); 3.814(2.8); 3.791(7.1);
3.767(7.3); 3.744(2.8); 2.496(2.4); 2.474(5.5); 2.452(5.7); 2.430(2.8); 2.410(2.5); 2.387(6.0); 2.362(6.4); 2.339(2.7); 2.176(0.6); 2.019(1.9); 1.993(5.0); 1.970(7.7); 1.947(5.0); 1.921(2.0); 1.821(2.4);
1.795(6.0); 1.774(6.3); 1.769(5.9); 1.748(5.0); 1.721(1.5); 1.625(0.8); 1.569(88.0); 1.518(0.7); 1.254(0.5); 0.146(0.4); 0.056(0.4); 0.000(81.2); −0.055(0.4); −0.149(0.4)
Example 446: ¹H-NMR(399.9 MHz, cdcl3):
δ = 7.708(14.0); 7.691(6.2); 7.687(15.6); 7.557(4.6); 7.540(5.4); 7.521(0.6); 7.409(16.0); 7.392(5.7); 7.388(13.4); 7.340(5.1); 7.337(5.3); 7.320(6.3); 7.317(6.8); 7.303(3.2); 7.300(3.1); 7.284(6.5);
7.282(6.0); 7.261(79.1); 7.186(3.6); 7.182(3.7); 7.167(5.2); 7.163(4.8); 7.148(2.2); 7.144(2.0); 6.997(0.4); 6.422(2.2); 6.402(2.2); 5.302(0.4); 4.864(0.8); 4.842(2.7); 4.820(3.8); 4.798(2.7); 4.777(0.8);
3.810(1.6); 3.786(3.9); 3.763(4.0); 3.739(1.6); 2.495(1.2); 2.473(2.9); 2.451(3.0); 2.428(1.5); 2.401(1.3); 2.377(3.1); 2.353(3.3); 2.330(1.4); 2.175(0.4); 2.011(1.1); 1.984(2.9); 1.961(4.4); 1.938(2.7);
1.935(2.6); 1.912(1.2); 1.824(1.4); 1.803(1.8); 1.797(3.4); 1.777(3.6); 1.771(3.3); 1.751(2.9); 1.745(1.3); 1.724(0.9); 1.566(80.1); 0.028(0.3); −0.001(54.3)
Example 447: ¹H-NMR(399.9 MHz, cdcl3):
δ = 7.768(13.1); 7.755(4.2); 7.750(16.0); 7.747(11.3); 7.585(5.6); 7.582(5.7); 7.566(6.5); 7.563(6.4); 7.520(0.9); 7.516(1.4); 7.512(2.5); 7.509(1.7); 7.500(1.8); 7.494(7.9); 7.488(2.6); 7.479(4.2); 7.476(6.8);
7.472(3.6); 7.443(12.0); 7.428(8.8); 7.424(15.7); 7.411(2.6); 7.407(6.0); 7.404(3.8); 7.389(0.3); 7.366(0.4); 7.348(0.7); 7.338(6.4); 7.335(6.9); 7.318(8.1); 7.315(9.0); 7.302(4.0); 7.300(3.8); 7.284(8.0);
7.281(7.2); 7.261(112.8); 7.181(4.7); 7.177(4.6); 7.162(6.5); 7.158(6.3); 7.143(2.7); 7.139(2.6); 6.997(0.6); 6.452(2.2); 6.434(2.3); 4.905(1.0); 4.882(3.3); 4.861(4.7); 4.839(3.4); 4.816(1.0); 3.808(2.0);
3.784(4.9); 3.761(5.0); 3.737(2.1); 2.502(1.4); 2.480(3.5); 2.459(3.5); 2.436(1.7); 2.411(1.5); 2.388(3.9); 2.363(4.0); 2.340(1.7); 2.013(1.6); 1.990(3.0); 1.987(3.9); 1.964(5.8); 1.941(3.5); 1.937(3.4);
1.915(1.7); 1.813(1.9); 1.793(2.2); 1.787(4.7); 1.766(4.6); 1.761(4.4); 1.740(3.9); 1.734(1.7); 1.714(1.3); 1.559(77.1); 1.294(0.4); 1.254(1.1); 0.146(0.3); 0.008(3.3); 0.000(86.2); −0.008(3.1); −0.149(0.3)
Example 448: ¹H-NMR(399.9 MHz, cdcl3):
δ = 7.492(0.4); 7.458(0.4); 7.455(0.4); 7.439(0.5); 7.436(0.5); 7.405(0.5); 7.402(0.4); 7.387(0.5); 7.383(0.4); 7.368(0.4); 7.317(0.3); 7.310(0.5); 7.306(0.8); 7.302(0.8); 7.265(8.4); 7.240(0.7); 7.221(0.5);
7.210(0.4); 7.206(0.7); 7.203(0.4); 1.613(16.0); −0.001(6.2)
Example 449: ¹H-NMR(399.9 MHz, cdcl3):
δ = 7.520(1.0); 7.509(4.5); 7.491(6.2); 7.445(5.5); 7.443(5.5); 7.425(9.0); 7.407(9.2); 7.389(10.2); 7.367(10.0); 7.349(16.0); 7.319(11.4); 7.301(14.8); 7.282(9.5); 7.261(122.4); 6.997(0.7); 5.679(1.8);
5.662(1.8); 5.139(0.9); 5.121(2.8); 5.102(4.1); 5.083(2.8); 5.063(0.8); 4.362(1.2); 4.343(2.9); 4.325(2.8); 4.307(1.1); 2.648(0.6); 2.644(0.7); 2.635(0.8); 2.630(1.5); 2.614(2.0); 2.609(1.9); 2.605(1.6);
2.600(1.8); 2.596(2.0); 2.584(1.6); 2.580(1.7); 2.575(0.8); 2.566(0.9); 2.561(0.8); 2.449(0.7); 2.436(1.2); 2.420(1.9); 2.405(2.6); 2.394(2.5); 2.381(2.9); 2.367(1.5); 2.354(3.7); 2.345(0.8); 2.334(3.6);
2.324(1.7); 2.313(1.4); 2.304(1.6); 2.283(0.6); 2.118(1.3); 2.100(2.1); 2.094(1.5); 2.091(1.4); 2.089(1.4); 2.074(2.3); 2.072(2.3); 2.058(1.2); 2.046(1.8); 2.028(0.9); 1.557(113.5); 1.416(0.7); 1.294(0.8);
1.255(1.4); 0.146(0.5); 0.069(0.4); 0.007(4.6); −0.001(93.3); −0.009(2.7); −0.150(0.4)
Example 450: ¹H-NMR(399.9 MHz, cdcl3):
δ = 7.952(0.7); 7.934(0.8); 7.761(13.9); 7.743(16.0); 7.740(12.5); 7.726(1.2); 7.706(0.8); 7.689(0.3); 7.560(10.0); 7.539(11.4); 7.521(3.1); 7.503(7.6); 7.484(6.5); 7.448(11.8); 7.429(15.5); 7.412(6.0);
7.340(11.3); 7.335(12.4); 7.270(10.4); 7.262(70.1); 7.250(7.2); 7.245(6.0); 6.998(0.4); 6.419(3.3); 6.398(3.3); 4.907(1.2); 4.885(3.8); 4.863(5.4); 4.841(3.9); 4.819(1.1); 3.731(2.2); 3.708(5.5); 3.684(5.5);
3.661(2.2); 2.484(1.9); 2.462(4.2); 2.441(4.4); 2.416(2.7); 2.412(2.5); 2.386(4.6); 2.362(4.8); 2.339(2.0); 2.023(1.6); 1.997(4.1); 1.973(6.0); 1.950(3.9); 1.924(1.5); 1.750(1.9); 1.729(2.6); 1.724(4.7);
1.703(4.8); 1.698(4.7); 1.677(4.1); 1.671(2.1); 1.650(1.4); 1.570(40.8); 1.434(0.4); 1.425(0.3); 1.253(2.3); 0.880(0.3); 0.146(0.4); −0.001(65.7)
Example 451: ¹H-NMR(399.9 MHz, cdcl3):
δ = 7.522(0.8); 7.459(2.5); 7.455(2.0); 7.445(11.1); 7.440(15.3); 7.426(8.2); 7.420(4.3); 7.406(15.3); 7.391(16.0); 7.387(11.0); 7.371(8.7); 7.363(12.0); 7.344(13.0); 7.325(3.8); 7.263(115.6); 6.999(0.6);
5.672(2.0); 5.653(2.1); 5.153(1.1); 5.133(3.0); 5.115(4.4); 5.095(3.1); 5.075(0.9); 4.293(1.2); 4.276(3.2); 4.258(3.2); 4.239(1.2); 2.646(0.6); 2.641(0.7); 2.627(1.9); 2.622(1.5); 2.612(2.3); 2.608(2.3);
2.602(1.8); 2.597(2.4); 2.593(2.3); 2.582(1.8); 2.578(2.2); 2.563(1.0); 2.559(0.8); 2.390(0.4); 2.374(4.3); 2.359(4.6); 2.353(8.8); 2.345(2.7); 2.335(9.0); 2.321(2.5); 2.315(2.2); 2.303(0.5); 2.098(1.3);
2.079(2.1); 2.069(1.5); 2.052(2.3); 2.049(2.3); 2.046(2.0); 2.036(1.2); 2.029(1.9); 2.008(0.8); 1.567(52.2); 1.285(0.7); 1.254(4.9); 0.880(0.7); 0.147(0.5); 0.000(108.2); −0.008(4.9); −0.149(0.5)
Example 452: ¹H-NMR(399.9 MHz, cdcl3):
δ = 7.702(14.0); 7.680(15.9); 7.535(7.7); 7.514(8.9); 7.416(16.0); 7.394(13.4); 7.343(9.3); 7.338(10.0); 7.272(10.7); 7.262(148.4); 7.253(8.1); 7.247(5.8); 7.201(0.4); 7.186(0.4); 6.998(0.8); 6.363(2.7);
6.341(2.7); 4.870(1.0); 4.847(2.8); 4.825(4.1); 4.804(3.0); 4.782(0.9); 3.736(1.8); 3.712(4.2); 3.689(4.3); 3.665(1.7); 2.482(1.4); 2.461(3.1); 2.437(3.3); 2.414(1.7); 2.406(1.6); 2.381(3.5); 2.356(3.9);
2.334(1.8); 2.021(1.2); 1.995(3.1); 1.972(4.6); 1.949(2.9); 1.922(1.2); 1.764(1.3); 1.738(3.5); 1.717(3.7); 1.712(3.5); 1.691(3.0); 1.664(1.0); 1.559(70.3); 1.442(0.4); 1.253(4.9); 0.880(0.8); 0.864(0.5);
0.842(0.5); 0.146(0.6); 0.000(140.0); −0.035(0.5); −0.041(0.5); −0.051(0.4); −0.061(0.5); −0.150(0.7)
Example 453: ¹H-NMR(399.9 MHz, cdcl3):
δ = 7.438(3.6); 7.433(4.0); 7.422(2.1); 7.401(4.2); 7.390(0.4); 7.371(3.1); 7.366(2.7); 7.350(2.3); 7.345(1.8); 7.327(16.0); 7.325(15.9); 7.310(0.7); 7.302(0.8); 7.263(34.2); 5.611(0.8); 5.592(0.8); 5.134(0.4);
5.114(1.1); 5.096(1.6); 5.077(1.1); 5.057(0.3); 4.286(0.4); 4.268(1.1); 4.250(1.1); 4.232(0.4); 2.623(0.7); 2.618(0.6); 2.607(0.9); 2.603(0.9); 2.597(0.6); 2.593(0.9); 2.588(0.9); 2.584(0.5); 2.578(0.7);
2.574(0.8); 2.568(0.4); 2.559(0.4); 2.554(0.4); 2.373(1.8); 2.351(2.7); 2.342(1.1); 2.333(3.8); 2.318(1.0); 2.315(0.9); 2.096(0.4); 2.076(0.8); 2.073(0.8); 2.066(0.5); 2.050(1.1); 2.043(0.8); 2.033(0.4);
2.026(0.7); 1.569(9.0); 1.253(1.0); 0.008(1.8); 0.000(32.0); −0.008(1.5)
Example 457: ¹H-NMR(399.9 MHz, cdcl3):
δ = 7.735(0.5); 7.621(0.4); 7.605(0.4); 7.522(1.0); 7.454(11.5); 7.449(12.4); 7.426(6.7); 7.423(5.0); 7.417(5.6); 7.414(5.1); 7.406(16.0); 7.400(7.3); 7.398(7.3); 7.395(6.5); 7.382(11.3); 7.376(13.7);
7.370(11.8); 7.366(7.0); 7.361(5.5); 7.356(4.9); 7.331(0.6); 7.299(5.3); 7.280(12.5); 7.263(163.0); 7.239(7.0); 7.235(9.8); 7.232(6.0); 7.219(3.6); 7.216(4.6); 7.204(0.6); 7.188(0.7); 7.167(0.7); 7.162(0.7);
7.155(0.9); 6.999(0.8); 5.606(2.5); 5.584(2.6); 5.304(1.1); 5.143(1.4); 5.123(3.5); 5.104(5.2); 5.085(3.6); 5.065(1.2); 4.297(1.4); 4.281(3.7); 4.263(3.7); 4.244(1.4); 3.501(0.5); 3.487(0.5); 2.754(0.4);
2.646(0.8); 2.641(0.8); 2.628(2.1); 2.623(2.0); 2.612(2.8); 2.608(2.8); 2.602(2.1); 2.597(3.0); 2.593(2.7); 2.582(2.3); 2.578(2.8); 2.573(1.3); 2.563(1.2); 2.559(1.0); 2.378(5.9); 2.357(8.9); 2.347(3.3);
2.339(12.7); 2.323(3.2); 2.320(3.0); 2.095(1.5); 2.075(2.7); 2.066(1.7); 2.049(2.8); 2.042(2.5); 2.025(2.4); 2.004(1.0); 1.694(0.6); 1.686(0.8); 1.676(0.6); 1.616(0.4); 1.561(122.6); 1.533(1.5); 1.497(0.4);
1.485(0.5); 1.254(3.0); 0.887(0.4); 0.147(0.7); 0.035(0.4); 0.008(8.0); 0.000(151.5); −0.008(6.8); −0.021(0.7); −0.032(0.4); −0.040(0.3); −0.074(0.6); −0.149(0.7)
Example 458: ¹H-NMR(399.9 MHz, cdcl3):
δ = 8.458(9.6); 8.455(9.5); 8.446(9.7); 8.444(9.1); 8.420(0.4); 8.187(3.2); 8.165(3.3); 7.811(10.5); 7.807(13.6); 7.790(12.3); 7.787(16.0); 7.666(0.3); 7.649(0.4); 7.605(8.9); 7.586(10.9); 7.574(9.3);
7.555(4.7); 7.522(1.4); 7.369(9.6); 7.357(9.4); 7.348(8.9); 7.337(8.4); 7.323(5.3); 7.304(8.9); 7.284(5.3); 7.263(189.8); 6.999(1.1); 5.304(1.2); 5.005(1.3); 4.982(4.3); 4.960(6.0); 4.938(4.3); 4.915(1.2);
3.860(1.8); 3.837(5.0); 3.813(5.0); 3.789(1.8); 2.486(2.2); 2.464(4.9); 2.442(5.2); 2.419(2.5); 2.361(1.9); 2.338(4.8); 2.314(5.0); 2.291(2.2); 2.268(0.5); 2.242(0.4); 2.221(0.6); 2.205(0.5); 2.074(0.6);
2.059(2.3); 2.032(4.7); 2.009(7.1); 1.986(4.5); 1.983(4.3); 1.960(2.0); 1.946(0.4); 1.924(0.4); 1.892(0.4); 1.845(2.1); 1.819(5.1); 1.798(5.5); 1.792(5.1); 1.772(4.5); 1.745(1.7); 1.644(0.8); 1.615(1.1);
1.561(172.1); 1.534(0.4); 1.494(0.6); 1.466(0.5); 1.444(0.4); 1.417(0.4); 1.350(0.3); 1.334(0.4); 1.295(0.5); 1.277(0.6); 1.254(2.2); 1.184(0.4); 0.972(0.4); 0.147(1.0); 0.070(3.5); 0.054(0.9); 0.000(178.2); −0.007
(6.4); −0.149(0.6)
Example 459: ¹H-NMR(400.0 MHz, DMSO):
δ = 8.630(3.4); 8.621(3.4); 7.626(2.6); 7.608(4.9); 7.585(2.0); 7.582(2.2); 7.567(4.3); 7.564(4.2); 7.549(4.6); 7.545(4.8); 7.527(3.4); 7.509(1.3); 7.304(9.0); 7.299(3.6); 7.288(4.5); 7.283(15.1); 7.277(2.4);
7.219(16.0); 7.203(6.2); 7.198(9.1); 6.660(2.7); 6.522(5.7); 6.384(3.0); 5.756(1.2); 3.323(33.5); 3.163(0.8); 3.151(1.5); 3.144(1.8); 3.133(2.7); 3.122(2.0); 3.115(1.5); 3.103(0.9); 2.675(0.4); 2.670(0.6);
2.666(0.4); 2.541(0.5); 2.510(37.0); 2.506(71.5); 2.501(92.6); 2.497(67.6); 2.493(33.7); 2.357(1.4); 2.339(3.3); 2.328(1.1); 2.318(3.3); 2.299(1.4); 1.348(1.1); 1.333(2.7); 1.328(1.9); 1.311(5.8); 1.293(4.5);
1.280(3.2); 1.265(0.8); 0.000(4.2)
Example 460: ¹H-NMR(400.0 MHz, DMSO):
δ = 8.185(5.0); 8.175(4.3); 7.767(0.3); 7.754(0.4); 7.699(1.9); 7.684(9.2); 7.671(8.0); 7.321(1.1); 7.298(2.9); 7.283(9.7); 7.279(7.4); 7.262(16.0); 7.241(4.8); 7.226(15.9); 7.205(7.3); 6.813(2.1); 6.798(9.5);
6.784(8.0); 5.756(0.9); 3.338(8.6); 3.322(37.4); 3.136(1.7); 3.124(2.8); 3.117(3.1); 3.106(3.8); 3.096(2.6); 3.088(2.0); 3.076(1.0); 2.890(0.4); 2.731(0.7); 2.671(1.6); 2.506(86.7); 2.502(98.9); 2.329(0.9);
2.290(2.3); 2.272(4.0); 2.250(3.6); 2.233(1.6); 1.343(2.5); 1.328(3.5); 1.323(3.8); 1.307(4.1); 1.286(2.8); 1.273(3.5); 1.258(4.8); 1.243(3.9); 1.228(1.5); 0.015(0.7); 0.000(3.4)
Example 461: ¹H-NMR(400.0 MHz, DMSO):
δ = 8.772(1.4); 8.763(1.3); 8.634(3.2); 8.628(3.8); 8.573(3.9); 8.567(3.1); 8.109(0.9); 8.094(0.9); 7.291(3.3); 7.269(6.6); 7.249(0.4); 7.230(6.3); 7.209(3.2); 6.610(1.2); 6.595(1.1); 3.798(0.5); 3.325(5.0);
3.210(0.4); 3.197(0.8); 3.190(0.9); 3.179(1.4); 3.167(0.9); 3.160(0.8); 3.148(0.4); 2.957(16.0); 2.676(0.4); 2.671(0.5); 2.506(54.5); 2.502(67.3); 2.498(48.6); 2.360(0.6); 2.341(1.4); 2.320(1.5); 2.302(0.6);
2.132(0.5); 1.381(0.7); 1.366(1.0); 1.360(1.1); 1.345(1.3); 1.339(0.9); 1.324(0.7); 1.267(0.9); 1.251(1.4); 1.237(1.4); 1.222(0.6); 0.000(5.9)
Example 462: ¹H-NMR(400.0 MHz, DMSO):
δ = 8.793(1.3); 8.781(1.3); 8.693(1.0); 8.683(1.0); 8.233(1.3); 8.231(1.2); 8.213(1.3); 8.210(1.3); 8.110(0.8); 8.095(0.8); 7.702(0.9); 7.689(0.9); 7.682(0.9); 7.669(0.8); 7.259(1.8); 7.253(0.8); 7.243(1.4);
7.237(6.3); 7.233(1.3); 7.219(5.7); 7.213(1.2); 7.202(0.9); 7.197(1.7); 6.611(1.2); 6.607(0.9); 6.598(0.9); 6.594(1.2); 3.328(1.0); 3.191(0.6); 3.183(0.6); 3.179(0.5); 3.172(1.0); 3.160(0.6); 3.153(0.6);
2.957(16.0); 2.511(14.0); 2.507(27.3); 2.502(35.2); 2.498(25.0); 2.493(12.1); 2.311(0.5); 2.293(1.0); 2.271(1.0); 2.252(0.5); 1.373(0.5); 1.358(0.8); 1.353(0.7); 1.338(1.0); 1.331(0.6); 1.316(0.6); 1.305(0.7);
1.291(1.0); 1.287(0.9); 1.274(1.0); 1.259(0.5); 0.000(4.1)
Example 463: ¹H-NMR(400.0 MHz, DMSO):
δ = 8.667(3.1); 8.657(3.0); 7.426(1.8); 7.411(2.1); 7.406(4.1); 7.390(4.3); 7.385(2.9); 7.370(2.8); 7.286(8.7); 7.282(3.4); 7.271(10.3); 7.265(16.0); 7.259(2.4); 7.251(4.8); 7.231(3.3); 7.229(3.2); 7.215(14.4);
7.209(8.7); 7.193(7.9); 7.188(3.4); 4.038(0.4); 4.020(0.4); 3.322(54.7); 3.197(0.8); 3.185(1.6); 3.177(1.6); 3.166(2.9); 3.155(1.8); 3.147(1.6); 3.135(0.9); 2.675(0.6); 2.670(0.8); 2.666(0.6); 2.541(0.6);
2.524(2.5); 2.510(47.9); 2.506(94.3); 2.501(122.1); 2.497(86.3); 2.492(40.6); 2.332(0.7); 2.326(1.7); 2.308(2.9); 2.286(2.9); 2.267(1.4); 1.989(1.7); 1.398(2.4); 1.339(1.6); 1.324(2.2); 1.318(2.4); 1.303(2.7);
1.296(1.9); 1.281(1.6); 1.190(1.9); 1.175(3.6); 1.160(2.8); 1.145(1.5); 0.146(0.4); 0.008(4.1); 0.000(99.9); −0.009(3.4); −0.150(0.5)
Example 464: ¹H-NMR(400.0 MHz, DMSO):
δ = 8.582(3.9); 8.572(3.9); 7.731(0.9); 7.711(1.2); 7.686(0.4); 7.629(6.0); 7.610(6.5); 7.370(0.5); 7.347(1.1); 7.336(0.5); 7.326(0.9); 7.317(0.5); 7.290(7.9); 7.269(16.0); 7.244(3.1); 7.231(15.9); 7.223(7.6);
7.210(8.3); 7.200(4.6); 7.167(2.8); 7.151(3.5); 7.147(3.8); 7.132(3.7); 7.111(1.5); 5.756(3.6); 3.653(0.4); 3.637(0.4); 3.619(0.4); 3.324(19.3); 3.297(0.6); 3.280(0.4); 3.192(0.8); 3.180(1.7); 3.172(2.0);
3.161(3.3); 3.150(2.0); 3.143(2.0); 3.131(1.0); 3.124(0.6); 3.105(0.5); 3.088(0.4); 3.070(0.5); 3.052(0.5); 3.034(0.3); 2.670(0.6); 2.505(77.2); 2.501(92.9); 2.497(66.8); 2.328(0.6); 2.312(1.4); 2.293(3.3);
2.272(3.3); 2.253(1.5); 1.358(1.6); 1.343(2.3); 1.338(2.9); 1.323(3.1); 1.316(2.0); 1.301(1.7); 1.236(0.5); 1.196(1.7); 1.178(3.4); 1.168(2.1); 1.155(3.8); 1.138(3.4); 1.123(1.6); 1.043(1.3); 1.026(2.8);
1.008(1.3); 0.000(9.5)
Example 465: ¹H-NMR(400.0 MHz, DMSO):
δ = 8.783(1.1); 8.774(1.1); 8.725(1.3); 8.720(1.4); 8.714(1.5); 8.709(1.4); 8.317(0.7); 8.101(0.7); 8.089(0.7); 7.633(0.6); 7.617(1.7); 7.600(1.5); 7.589(1.4); 7.581(0.5); 7.569(0.5); 7.323(0.4); 7.316(3.1);
7.312(1.2); 7.300(1.3); 7.295(4.8); 7.289(0.8); 7.266(0.4); 7.255(0.3); 7.226(0.8); 7.220(4.4); 7.199(2.8); 6.585(1.1); 6.569(1.1); 6.530(0.9); 6.395(1.8); 6.260(1.0); 5.757(0.4); 3.327(8.2); 3.160(0.5);
3.152(0.6); 3.142(0.9); 3.132(0.6); 3.124(0.5); 2.941(16.0); 2.511(7.4); 2.506(15.0); 2.502(19.8); 2.497(14.4); 2.493(7.0); 2.384(0.5); 2.366(1.0); 2.344(1.0); 2.326(0.5); 2.105(0.7); 1.600(0.5); 1.368(0.5);
1.352(0.8); 1.348(0.7); 1.331(1.0); 1.326(0.6); 1.310(0.6); 1.290(0.6); 1.273(1.0); 1.260(1.0); 1.245(0.5); 0.951(0.8); 0.934(0.8); 0.000(2.5)
Example 466: ¹H-NMR(400.0 MHz, DMSO):
δ = 8.692(2.7); 8.682(2.7); 7.340(0.3); 7.318(0.4); 7.285(7.2); 7.280(2.7); 7.268(3.4); 7.263(12.1); 7.257(1.7); 7.246(0.5); 7.236(0.6); 7.222(4.3); 7.199(16.0); 7.179(9.0); 5.756(1.6); 3.322(41.4); 3.173(0.7);
3.161(1.2); 3.153(1.4); 3.143(2.3); 3.132(1.5); 3.124(1.3); 3.112(0.7); 3.014(0.3); 2.675(0.4); 2.670(0.6); 2.666(0.4); 2.510(35.0); 2.506(69.2); 2.501(90.8); 2.497(65.5); 2.493(31.9); 2.332(0.4); 2.328(0.6);
2.319(1.2); 2.301(2.3); 2.279(2.3); 2.261(1.1); 1.351(1.3); 1.335(1.8); 1.330(2.0); 1.314(2.2); 1.308(1.6); 1.293(1.3); 1.160(1.4); 1.144(2.3); 1.131(2.3); 1.115(1.2); 0.008(0.4); 0.000(9.8); −0.008(0.4)
Example 467: ¹H-NMR(400.0 MHz, DMSO):
δ = 8.650(4.0); 8.641(3.9); 8.315(0.4); 7.971(4.9); 7.968(4.7); 7.952(5.8); 7.948(5.5); 7.699(2.0); 7.696(2.3); 7.680(5.5); 7.677(5.5); 7.662(4.5); 7.658(4.0); 7.645(4.0); 7.641(4.4); 7.625(4.8); 7.621(5.0);
7.606(2.1); 7.602(1.8); 7.339(10.6); 7.335(4.1); 7.323(5.6); 7.318(16.0); 7.312(2.4); 7.233(14.6); 7.212(9.8); 6.980(5.2); 6.977(5.4); 6.962(4.8); 6.958(4.8); 5.755(0.5); 3.322(148.3); 3.150(0.9); 3.138(1.8);
3.130(2.2); 3.120(3.3); 3.109(2.3); 3.101(1.8); 3.090(1.0); 2.675(0.9); 2.671(1.3); 2.666(0.9); 2.540(1.0); 2.510(81.0); 2.506(151.4); 2.501(191.4); 2.497(138.4); 2.493(68.4); 2.332(2.3); 2.324(1.3);
2.314(3.4); 2.291(3.4); 2.273(1.6); 1.369(1.7); 1.354(2.7); 1.349(2.8); 1.332(3.2); 1.327(2.3); 1.312(1.8); 1.228(2.1); 1.212(3.4); 1.199(3.4); 1.184(1.6); 0.146(0.9); 0.032(0.3); 0.000(196.3); −0.008(9.2); −0.150
(0.9)
Example 468: ¹H-NMR(399.9 MHz, cdcl3):
δ = 7.897(5.9); 7.533(3.0); 7.521(0.4); 7.513(3.4); 7.328(4.3); 7.323(4.8); 7.262(34.6); 7.253(3.1); 7.248(2.5); 7.232(2.1); 7.227(2.0); 6.949(1.7); 6.813(3.6); 6.678(1.8); 6.622(0.8); 4.820(0.9); 4.798(1.4);
4.776(1.0); 3.911(16.0); 3.681(0.6); 3.658(1.6); 3.634(1.5); 3.611(0.7); 2.441(0.5); 2.419(1.2); 2.412(0.9); 2.405(0.9); 2.398(1.2); 2.394(1.4); 2.386(1.4); 2.377(1.0); 2.360(1.3); 2.337(0.5); 1.992(0.4);
1.965(1.2); 1.942(1.7); 1.919(1.0); 1.915(1.0); 1.892(0.4); 1.715(0.5); 1.693(0.7); 1.687(1.3); 1.667(1.3); 1.661(1.4); 1.641(1.2); 1.635(0.6); 1.614(0.4); 1.568(23.8); 0.008(1.5); 0.000(32.0); −0.008(1.5)
Example 469: ¹H-NMR(399.9 MHz, cdcl3):
δ = 7.793(6.0); 7.521(0.4); 7.368(4.4); 7.363(5.4); 7.339(4.6); 7.311(3.5); 7.306(3.0); 7.290(1.7); 7.285(1.6); 7.262(69.2); 6.998(0.4); 6.666(1.7); 6.530(3.4); 6.395(1.7); 6.059(0.7); 5.053(0.8); 5.035(1.2);
5.018(0.8); 4.239(0.5); 4.220(1.3); 4.200(1.4); 4.181(0.5); 3.860(16.0); 2.594(0.7); 2.590(0.5); 2.586(0.5); 2.582(0.7); 2.578(0.8); 2.575(0.8); 2.571(0.7); 2.567(0.8); 2.564(0.8); 2.560(0.8); 2.556(0.5);
2.548(0.8); 2.546(0.8); 2.531(0.4); 2.527(0.4); 2.401(0.5); 2.387(0.7); 2.372(1.0); 2.363(0.9); 2.355(0.6); 2.348(1.1); 2.340(0.6); 2.337(0.6); 2.331(0.6); 2.319(1.3); 2.316(0.9); 2.310(0.4); 2.302(1.0);
2.298(1.3); 2.294(0.7); 2.289(0.7); 2.285(0.5); 2.281(0.5); 2.277(0.5); 2.272(0.5); 2.268(0.6); 2.061(0.5); 2.046(0.8); 2.036(0.6); 2.031(0.9); 2.021(0.9); 2.019(0.8); 2.016(0.9); 2.007(0.7); 2.001(0.5);
1.992(0.6); 1.562(46.7); 1.253(0.5); 0.023(0.3); 0.022(0.3); 0.021(0.4); 0.018(0.5); 0.017(0.5); 0.008(3.1); 0.000(63.9); −0.008(2.8); −0.016(0.4)
Example 470: ¹H-NMR(399.9 MHz, cdcl3):
δ = 8.774(6.4); 8.771(6.5); 8.762(6.6); 8.759(6.3); 7.917(6.1); 7.915(6.1); 7.898(6.8); 7.895(6.6); 7.572(6.2); 7.560(6.3); 7.552(5.7); 7.541(5.4); 7.521(1.4); 7.513(9.8); 7.492(11.6); 7.391(0.4); 7.385(0.4);
7.362(14.4); 7.357(16.0); 7.297(8.7); 7.292(7.8); 7.276(8.2); 7.271(9.1); 7.262(130.5); 6.998(0.7); 6.096(2.9); 6.075(3.0); 4.880(1.1); 4.858(3.5); 4.836(5.0); 4.814(3.6); 4.792(1.1); 3.711(2.0); 3.687(5.1);
3.664(5.1); 3.640(2.1); 2.502(1.5); 2.480(3.5); 2.459(3.6); 2.455(3.5); 2.434(3.4); 2.407(3.9); 2.382(4.2); 2.360(1.8); 2.023(1.4); 2.000(3.1); 1.996(3.7); 1.973(5.3); 1.950(3.4); 1.946(3.3); 1.924(1.4);
1.762(1.6); 1.742(2.1); 1.736(4.3); 1.716(4.2); 1.710(4.4); 1.689(4.0); 1.683(2.0); 1.663(1.2); 1.559(84.0); 1.253(0.6); 0.146(0.4); 0.008(4.8); 0.000(122.4); −0.009(6.9); −0.050(0.4); −0.150(0.6)
Example 471: ¹H-NMR(399.9 MHz, cdcl3):
δ = 7.586(8.0); 7.584(7.9); 7.575(10.8); 7.566(9.5); 7.564(9.1); 7.554(12.0); 7.540(7.0); 7.536(7.2); 7.521(8.6); 7.517(8.2); 7.372(4.3); 7.369(4.3); 7.353(10.4); 7.350(12.0); 7.348(15.4); 7.342(16.0);
7.335(6.0); 7.332(5.1); 7.291(6.4); 7.284(10.0); 7.279(8.3); 7.271(9.6); 7.262(98.4); 7.252(4.8); 7.248(3.6); 7.162(0.5); 6.998(0.5); 6.282(2.6); 6.260(2.6); 4.917(1.0); 4.895(3.6); 4.873(5.1); 4.851(3.6);
4.828(1.1); 3.721(2.2); 3.698(5.1); 3.674(5.2); 3.651(1.9); 2.494(1.5); 2.472(3.6); 2.451(3.7); 2.447(3.7); 2.438(2.4); 2.428(2.4); 2.414(3.8); 2.390(4.1); 2.367(1.7); 2.046(1.7); 2.023(3.1); 2.019(3.6);
1.996(5.6); 1.973(3.5); 1.970(3.0); 1.947(1.2); 1.736(1.5); 1.715(2.3); 1.709(4.5); 1.689(4.4); 1.683(4.4); 1.662(3.8); 1.656(1.8); 1.636(1.2); 1.559(66.5); 0.146(0.4); 0.008(4.7); 0.000(86.2); −0.008(3.6); −0.149
(0.4)
Example 472: ¹H-NMR(399.9 MHz, cdcl3):
δ = 7.521(0.9); 7.497(7.8); 7.494(8.0); 7.477(9.2); 7.474(9.2); 7.418(14.9); 7.412(16.0); 7.382(6.3); 7.377(7.2); 7.367(8.9); 7.363(11.1); 7.358(10.9); 7.346(15.3); 7.333(1.0); 7.308(7.4); 7.306(15.3);
7.300(10.3); 7.290(10.9); 7.287(11.4); 7.279(6.8); 7.271(8.2); 7.268(10.8); 7.262(148.5); 7.229(6.7); 7.225(6.4); 7.210(7.5); 7.205(6.9); 7.191(3.7); 7.187(3.5); 6.998(0.8); 5.759(2.4); 5.741(2.5); 5.131(1.0);
5.113(3.1); 5.093(4.2); 5.076(3.0); 5.058(1.0); 4.291(1.7); 4.272(4.4); 4.253(4.4); 4.234(1.6); 2.665(0.9); 2.661(0.9); 2.645(2.2); 2.640(1.6); 2.635(1.5); 2.631(2.4); 2.625(2.7); 2.621(2.2); 2.616(2.6);
2.613(2.6); 2.610(2.7); 2.606(1.7); 2.598(2.6); 2.595(2.6); 2.580(1.4); 2.576(1.3); 2.444(0.8); 2.427(1.4); 2.413(2.5); 2.398(3.3); 2.389(3.0); 2.381(2.2); 2.373(5.0); 2.355(5.4); 2.351(3.3); 2.346(1.3);
2.338(3.5); 2.334(4.4); 2.330(2.2); 2.325(2.2); 2.321(1.6); 2.317(1.8); 2.313(1.7); 2.307(1.6); 2.304(1.9); 2.287(0.7); 2.283(0.7); 2.129(1.6); 2.112(2.8); 2.105(1.9); 2.103(1.9); 2.097(2.8); 2.088(2.8);
2.085(2.7); 2.082(2.8); 2.073(2.6); 2.067(1.7); 2.065(1.6); 2.058(2.2); 2.048(1.1); 2.043(1.1); 1.559(101.2); 1.279(0.4); 1.261(0.8); 1.254(0.9); 1.243(0.4); 0.883(0.5); 0.146(0.6); 0.050(0.4); 0.041(0.4);
0.008(6.7); 0.000(137.5); −0.008(6.0); −0.149(0.6)
Example 473: ¹H-NMR(399.9 MHz, cdcl3):
δ = 7.521(0.9); 7.428(11.8); 7.422(12.6); 7.349(5.6); 7.329(13.0); 7.317(3.6); 7.312(3.4); 7.300(10.4); 7.295(13.9); 7.291(3.1); 7.280(7.0); 7.274(8.1); 7.262(172.6); 6.998(1.0); 6.887(1.4); 6.883(1.9);
6.877(10.4); 6.865(1.7); 6.857(16.0); 6.851(2.6); 6.848(1.7); 6.836(8.7); 6.830(1.7); 6.827(1.2); 5.588(1.6); 5.574(1.7); 5.155(0.8); 5.136(2.5); 5.118(3.4); 5.099(2.5); 5.080(0.8); 4.287(1.3); 4.269(3.4);
4.250(3.3); 4.231(1.2); 2.658(0.7); 2.654(0.7); 2.642(1.0); 2.638(1.8); 2.628(1.1); 2.624(2.0); 2.618(2.1); 2.614(1.8); 2.609(2.1); 2.603(2.1); 2.599(1.2); 2.588(2.1); 2.584(1.0); 2.573(1.0); 2.569(0.9);
2.416(0.5); 2.400(0.9); 2.392(0.7); 2.385(2.2); 2.369(3.8); 2.365(2.6); 2.361(3.0); 2.354(2.1); 2.347(7.2); 2.329(4.8); 2.326(4.4); 2.317(1.3); 2.308(1.5); 2.305(1.4); 2.296(1.2); 2.278(0.4); 2.275(0.4);
2.113(1.2); 2.095(2.1); 2.088(1.3); 2.080(2.0); 2.071(2.1); 2.068(2.1); 2.065(2.1); 2.057(1.6); 2.048(1.5); 2.041(1.6); 2.025(0.8); 1.555(49.7); 1.254(0.8); 0.146(0.7); 0.034(0.4); 0.008(6.6); 0.000(161.6); −0.008
(5.6); −0.149(0.6)
Example 474: ¹H-NMR(399.9 MHz, cdcl3):
δ = 7.699(5.5); 7.680(7.3); 7.607(1.8); 7.591(5.8); 7.584(2.7); 7.572(6.6); 7.569(5.5); 7.553(5.9); 7.545(10.8); 7.536(14.6); 7.524(13.0); 7.353(13.9); 7.347(16.0); 7.317(0.4); 7.290(8.4); 7.285(7.6);
7.269(11.4); 7.262(217.6); 7.244(0.5); 6.998(1.2); 6.055(2.4); 6.034(2.5); 5.303(4.8); 4.910(0.9); 4.888(3.2); 4.866(4.6); 4.844(3.3); 4.822(1.0); 3.675(1.9); 3.651(4.6); 3.628(4.8); 3.604(1.8); 2.491(1.3);
2.469(3.3); 2.448(3.3); 2.444(3.2); 2.429(2.8); 2.425(2.8); 2.406(3.5); 2.381(3.8); 2.358(1.6); 2.002(1.4); 1.979(2.7); 1.975(3.4); 1.952(5.3); 1.929(3.2); 1.926(3.0); 1.902(1.1); 1.724(1.3); 1.704(2.0);
1.697(4.2); 1.677(3.9); 1.671(4.1); 1.651(3.6); 1.645(1.7); 1.624(1.2); 1.556(181.5); 1.456(0.3); 1.253(0.8); 0.146(0.7); 0.008(6.8); 0.000(198.7); −0.008(8.6); −0.101(0.4); −0.150(0.8)
Example 476: ¹H-NMR(601.6 MHz, DMSO):
δ = 8.458(1.0); 8.444(1.0); 7.699(1.1); 7.687(1.4); 7.615(0.4); 7.603(1.2); 7.592(1.0); 7.583(3.1); 7.580(3.4); 7.569(1.1); 7.557(0.4); 7.509(1.3); 7.495(2.7); 7.473(1.8); 7.469(1.6); 7.459(0.9); 7.455(0.9);
6.881(1.2); 6.869(1.2); 4.900(0.4); 4.894(0.4); 4.886(0.5); 4.880(0.4); 4.130(0.7); 4.116(0.7); 3.340(7.0); 2.508(4.8); 2.505(10.4); 2.502(14.3); 2.500(10.5); 2.497(4.9); 2.451(0.5); 2.440(0.9); 2.436(1.1);
2.431(0.8); 2.426(1.3); 2.414(0.7); 2.188(0.5); 2.183(0.5); 2.015(0.4); 2.010(0.4); 2.002(0.6); 1.996(0.3); 1.397(16.0); 0.005(0.5); 0.000(13.1); −0.006(0.5)
Example 477: ¹H-NMR(399.8 MHz, cdcl3):
δ = 7.780(5.4); 7.767(5.8); 7.759(6.0); 7.746(5.5); 7.715(9.1); 7.710(15.0); 7.706(9.1); 7.589(6.3); 7.586(8.3); 7.583(5.8); 7.570(7.6); 7.567(9.6); 7.520(1.3); 7.501(0.4); 7.476(5.1); 7.474(5.6); 7.471(5.3);
7.468(4.7); 7.456(7.6); 7.454(7.7); 7.451(7.8); 7.448(6.1); 7.375(11.1); 7.356(16.0); 7.335(11.5); 7.328(8.4); 7.312(6.7); 7.305(11.8); 7.283(8.0); 7.277(7.1); 7.261(206.9); 6.997(1.2); 6.294(3.6); 6.272(3.7);
4.924(1.4); 4.901(4.6); 4.879(6.4); 4.857(4.5); 4.836(1.3); 3.738(1.9); 3.714(5.0); 3.691(5.1); 3.668(1.8); 3.492(0.4); 2.498(2.1); 2.476(5.0); 2.455(5.4); 2.433(2.6); 2.333(2.0); 2.310(5.1); 2.287(5.4);
2.263(2.1); 1.999(2.1); 1.973(5.3); 1.950(7.6); 1.927(4.8); 1.923(4.6); 1.901(2.6); 1.850(2.3); 1.824(5.4); 1.803(5.4); 1.798(5.1); 1.777(4.5); 1.751(1.3); 1.547(232.9); 1.494(0.6); 1.424(0.4); 1.255(1.4);
0.146(0.8); 0.008(9.7); 0.000(194.7); −0.008(9.1); −0.058(0.4); −0.124(0.4); −0.149(1.0)
Example 478: ¹H-NMR(399.8 MHz, cdcl3):
δ = 8.458(9.9); 8.454(9.6); 8.446(10.2); 8.443(9.1); 8.171(3.4); 8.146(3.5); 7.814(14.3); 7.811(11.1); 7.800(6.8); 7.793(16.0); 7.790(13.5); 7.779(5.7); 7.520(1.1); 7.372(9.9); 7.360(9.8); 7.351(9.1);
7.340(8.7); 7.316(6.1); 7.309(8.1); 7.287(11.1); 7.261(180.4); 7.249(5.7); 7.163(0.3); 6.997(1.0); 4.970(1.3); 4.948(4.4); 4.925(6.3); 4.904(4.6); 4.881(1.4); 3.797(2.0); 3.774(5.0); 3.751(5.1); 3.727(1.9);
3.492(0.5); 2.468(2.2); 2.445(5.0); 2.423(5.4); 2.399(2.5); 2.350(1.9); 2.326(4.9); 2.302(5.2); 2.279(2.2); 2.058(2.2); 2.031(5.1); 2.008(7.7); 1.985(4.9); 1.958(1.9); 1.802(2.2); 1.776(5.3); 1.755(5.7);
1.750(5.2); 1.729(4.6); 1.703(1.4); 1.679(0.4); 1.639(0.5); 1.547(138.9); 1.255(2.0); 1.199(0.4); 0.881(0.4); 0.843(0.5); 0.810(0.3); 0.146(0.8); 0.070(0.7); 0.000(172.8); −0.008(6.9); −0.150(0.7); −1.602(0.3)
Example 479: ¹H-NMR(399.8 MHz, cdcl3):
δ = 14.624(0.5); 8.256(15.5); 8.254(14.4); 8.245(15.7); 7.739(14.2); 7.737(13.0); 7.718(16.0); 7.717(14.1); 7.701(9.2); 7.688(9.3); 7.679(10.5); 7.666(9.7); 7.550(5.3); 7.536(5.1); 7.521(4.3); 7.490(1.1);
7.383(10.3); 7.376(12.3); 7.360(10.4); 7.353(12.5); 7.331(6.6); 7.324(5.9); 7.310(11.3); 7.304(9.6); 7.285(19.8); 7.273(20.0); 7.261(317.4); 7.260(320.9); 7.175(0.8); 6.996(2.0); 4.989(1.9); 4.970(6.1);
4.952(8.5); 4.935(6.3); 4.915(2.0); 4.365(2.7); 4.347(7.4); 4.328(7.4); 4.308(2.8); 3.504(0.8); 3.492(0.9); 3.375(0.6); 2.802(0.7); 2.695(1.8); 2.676(4.9); 2.658(5.7); 2.643(5.4); 2.628(5.7); 2.609(3.1);
2.492(1.5); 2.475(2.6); 2.461(5.4); 2.445(7.4); 2.435(7.0); 2.429(7.6); 2.420(9.0); 2.408(10.1); 2.389(8.4); 2.369(3.9); 2.357(3.4); 2.336(1.5); 2.236(0.6); 2.210(3.7); 2.194(5.6); 2.184(4.3); 2.178(6.1);
2.165(6.0); 2.154(5.1); 2.139(4.1); 2.122(2.3); 2.056(0.6); 2.009(0.6); 1.716(0.5); 1.676(0.8); 1.653(1.0); 1.553(175.0); 1.324(0.8); 1.318(0.8); 1.284(1.7); 1.255(3.0); 1.203(0.8); 0.880(0.7); 0.862(0.7);
0.843(0.8); 0.822(0.7); 0.146(1.6); 0.069(0.7); 0.000(295.2); −0.001(308.5); −0.008(16.3); −0.150(1.7)
Example 480: ¹H-NMR(399.8 MHz, cdcl3):
δ = 14.023(0.5); 8.704(12.5); 8.701(12.5); 8.114(0.6); 8.072(3.7); 8.049(16.0); 8.046(15.0); 7.786(5.4); 7.773(5.6); 7.765(6.0); 7.752(5.6); 7.589(0.5); 7.519(3.1); 7.376(0.5); 7.358(0.7); 7.323(6.3);
7.316(8.8); 7.300(8.5); 7.294(11.8); 7.276(10.6); 7.260(513.2); 6.996(3.0); 4.929(1.2); 4.907(4.6); 4.885(6.4); 4.864(4.7); 4.841(1.3); 3.807(1.9); 3.783(5.1); 3.761(5.1); 3.737(2.0); 3.581(0.5); 3.553(0.6);
3.495(0.7); 3.404(0.5); 2.486(2.2); 2.462(5.2); 2.441(5.3); 2.417(2.4); 2.359(1.9); 2.335(5.0); 2.310(5.2); 2.289(2.2); 2.071(2.5); 2.044(6.7); 2.021(8.0); 1.998(4.8); 1.994(4.8); 1.971(2.1); 1.845(2.3);
1.819(5.2); 1.798(5.5); 1.792(5.4); 1.771(4.3); 1.745(1.4); 1.535(221.3); 1.290(0.6); 1.280(0.6); 1.259(1.8); 1.241(0.7); 0.850(0.7); 0.146(2.6); 0.126(0.6); 0.100(0.7); 0.008(28.3); 0.000(613.2); −0.009
(20.9); −0.150(2.4)
Example 481: ¹H-NMR(399.9 MHz, cdcl3):
δ = 8.705(5.8); 8.702(6.0); 8.693(5.9); 7.690(0.4); 7.659(5.4); 7.642(6.7); 7.639(6.6); 7.518(1.3); 7.498(6.3); 7.486(6.5); 7.478(5.4); 7.467(4.9); 7.421(12.1); 7.416(12.8); 7.363(1.1); 7.335(5.3); 7.314(16.0);
7.298(11.7); 7.293(10.7); 7.277(5.1); 7.272(5.8); 7.259(202.3); 6.995(1.1); 5.400(2.5); 5.381(2.7); 5.092(1.0); 5.074(3.0); 5.056(4.0); 5.038(2.9); 5.021(0.9); 4.281(1.6); 4.262(4.5); 4.243(4.4); 4.223(1.6);
2.671(0.8); 2.667(0.9); 2.650(2.3); 2.637(2.2); 2.632(2.7); 2.619(2.7); 2.616(2.6); 2.612(1.7); 2.603(2.6); 2.585(1.3); 2.582(1.3); 2.451(0.8); 2.434(1.5); 2.420(2.4); 2.404(3.5); 2.396(2.9); 2.387(2.2);
2.380(4.9); 2.361(5.1); 2.357(3.2); 2.344(3.3); 2.340(4.1); 2.335(2.3); 2.331(2.1); 2.327(1.7); 2.323(1.8); 2.319(1.7); 2.314(1.5); 2.310(1.8); 2.292(0.7); 2.289(0.7); 2.114(1.5); 2.099(2.6); 2.083(2.9);
2.075(2.7); 2.069(2.8); 2.060(2.5); 2.055(1.8); 2.045(2.1); 2.029(1.1); 1.531(51.7); 1.255(1.1); 0.146(0.8); 0.050(0.3); 0.008(9.6); 0.000(214.7); −0.009(10.7); −0.054(0.4); −0.150(0.9)
Example 482: ¹H-NMR(399.9 MHz, cdcl3):
δ = 7.549(0.4); 7.528(0.5); 7.363(0.3); 7.348(0.7); 7.343(0.8); 7.283(0.4); 7.278(0.4); 7.268(7.6); 6.960(0.5); 6.940(0.8); 6.919(0.4); 1.675(16.0); 1.666(0.4); 0.008(0.3); 0.000(7.3)
Example 483: ¹H-NMR(399.8 MHz, cdcl3):
δ = 10.210(0.3); 8.702(0.3); 8.510(15.8); 8.507(16.0); 7.980(15.5); 7.977(15.3); 7.690(6.8); 7.677(7.2); 7.669(7.8); 7.656(7.7); 7.599(0.3); 7.519(1.7); 7.415(4.2); 7.401(11.9); 7.394(13.4); 7.378(9.0);
7.371(10.3); 7.346(5.2); 7.339(4.2); 7.325(8.0); 7.319(6.7); 7.305(4.5); 7.298(3.9); 7.260(281.3); 7.213(0.5); 7.161(0.3); 7.140(0.4); 6.996(1.6); 4.994(1.5); 4.974(4.7); 4.956(6.6); 4.938(5.0); 4.919(1.5);
4.380(2.1); 4.360(5.6); 4.341(5.5); 4.322(2.0); 3.511(0.4); 3.427(0.3); 3.300(0.4); 2.707(1.4); 2.691(3.6); 2.670(4.5); 2.666(3.6); 2.661(4.0); 2.658(4.1); 2.655(4.4); 2.643(4.2); 2.640(4.2); 2.624(2.3);
2.621(2.1); 2.499(0.9); 2.483(2.0); 2.468(4.3); 2.452(6.2); 2.443(8.2); 2.436(3.9); 2.425(11.5); 2.411(5.1); 2.403(7.7); 2.394(2.7); 2.382(2.8); 2.373(2.3); 2.354(0.9); 2.213(2.7); 2.196(4.5); 2.187(3.0);
2.181(4.6); 2.172(4.3); 2.157(3.9); 2.150(2.4); 2.141(3.2); 2.126(1.6); 2.045(0.4); 1.536(133.2); 1.302(0.5); 1.255(1.4); 0.880(0.4); 0.862(0.4); 0.835(0.4); 0.146(1.7); 0.127(0.4); 0.096(0.4); 0.076(0.4);
0.008(16.2); −0.001(325.8); −0.009(10.7); −0.150(1.4); −1.537(0.3)
Example 484: ¹H-NMR(299.9 MHz, cdcl3):
δ = 7.852(4.9); 7.773(1.2); 7.755(1.3); 7.744(1.3); 7.727(1.2); 7.318(1.3); 7.309(1.9); 7.287(2.1); 7.279(2.6); 7.258(43.3); 7.231(0.8); 7.221(0.6); 6.975(1.9); 6.794(3.8); 6.613(2.0); 6.550(0.7); 4.881(0.9);
4.852(1.3); 4.822(0.9); 3.894(16.0); 3.743(0.8); 3.701(0.4); 3.671(1.1); 3.639(1.2); 3.610(0.4); 2.469(0.5); 2.439(1.2); 2.411(1.2); 2.380(0.6); 2.330(0.5); 2.300(1.1); 2.269(1.2); 2.236(0.5); 1.980(0.5);
1.945(1.2); 1.914(1.7); 1.884(1.0); 1.879(1.0); 1.850(0.6); 1.797(0.6); 1.763(1.3); 1.736(1.3); 1.729(1.2); 1.701(1.1); 1.666(0.4); 1.554(3.3); 0.011(2.3); 0.000(43.3); −0.011(1.6)
Example 485: ¹H-NMR(299.9 MHz, cdcl3):
δ = 7.799(5.0); 7.651(1.2); 7.633(1.3); 7.622(1.6); 7.604(1.6); 7.390(1.4); 7.382(1.8); 7.360(1.4); 7.351(1.8); 7.321(0.9); 7.312(0.9); 7.294(1.5); 7.284(1.4); 7.258(33.2); 6.662(1.7); 6.481(3.4); 6.300(1.7);
6.077(0.8); 4.986(1.0); 4.961(1.4); 4.937(1.0); 4.324(0.4); 4.297(1.1); 4.273(1.1); 3.837(16.0); 2.657(0.8); 2.631(1.0); 2.624(0.7); 2.617(0.9); 2.610(0.9); 2.590(0.9); 2.570(0.4); 2.566(0.4); 2.404(1.2);
2.386(1.4); 2.376(3.4); 2.351(2.9); 2.333(1.2); 2.324(0.9); 2.308(0.3); 2.161(0.5); 2.138(0.8); 2.128(0.7); 2.118(0.8); 2.102(0.9); 2.092(0.7); 2.067(0.6); 2.041(0.5); 1.529(20.3); 1.257(0.9); 0.010(1.5);
0.000(34.0)
Example 486: ¹H-NMR(399.8 MHz, cdcl3):
δ = 7.751(4.9); 7.519(0.4); 7.260(77.9); 7.172(0.5); 7.156(1.0); 7.151(0.9); 7.140(0.6); 7.135(2.0); 7.130(0.7); 7.119(0.9); 7.114(1.2); 7.098(0.6); 6.996(0.5); 6.870(0.4); 6.860(2.6); 6.850(0.5); 6.838(4.7);
6.828(0.6); 6.818(2.2); 6.807(0.4); 6.769(1.8); 6.633(3.6); 6.533(0.7); 6.497(1.9); 5.077(0.7); 5.058(1.1); 5.038(0.7); 4.356(0.4); 4.353(0.4); 4.336(1.0); 4.314(1.2); 4.292(0.5); 3.853(16.0); 2.606(0.7);
2.582(0.8); 2.559(0.4); 2.528(0.4); 2.515(0.6); 2.500(0.9); 2.487(0.9); 2.473(0.8); 2.450(0.6); 2.430(0.7); 2.411(1.0); 2.381(0.9); 2.373(0.7); 2.364(1.0); 2.356(0.8); 2.339(1.1); 2.325(0.5); 2.313(0.4);
1.545(77.7); 0.146(0.3); 0.008(3.4); 0.000(81.6); −0.009(2.6); −0.014(0.3)
Example 487: ¹H-NMR(399.8 MHz, cdcl3):
δ = 8.755(10.8); 8.745(11.2); 7.860(10.5); 7.841(11.9); 7.765(7.0); 7.751(7.9); 7.743(8.3); 7.730(7.8); 7.549(10.1); 7.537(10.6); 7.529(9.6); 7.518(8.9); 7.351(8.4); 7.345(11.0); 7.328(9.8); 7.322(16.0);
7.303(9.9); 7.297(7.0); 7.282(5.3); 7.275(4.2); 7.261(53.9); 6.997(0.4); 6.044(5.2); 6.023(5.3); 4.937(1.9); 4.914(6.3); 4.892(9.0); 4.870(6.3); 4.848(1.8); 3.716(2.6); 3.692(7.3); 3.669(7.4); 3.646(2.8);
2.511(2.9); 2.489(6.9); 2.466(7.3); 2.444(3.3); 2.340(2.7); 2.317(7.0); 2.293(7.3); 2.270(3.0); 2.041(0.6); 1.996(2.6); 1.969(6.4); 1.946(10.0); 1.923(6.3); 1.920(5.8); 1.897(2.8); 1.835(3.0); 1.809(7.0);
1.789(7.6); 1.783(6.6); 1.763(5.8); 1.736(1.7); 1.567(15.6); 1.257(1.0); 0.146(0.4); 0.000(56.9); −0.001(58.0); −0.008(2.5)
Example 489: ¹H-NMR(399.8 MHz, cdcl3):
δ = 8.800(0.4); 8.790(0.4); 8.692(12.2); 8.681(12.7); 7.654(11.5); 7.635(14.3); 7.614(7.8); 7.601(8.4); 7.593(9.7); 7.579(8.9); 7.560(0.7); 7.546(0.6); 7.521(1.0); 7.486(11.0); 7.474(11.5); 7.467(10.2);
7.455(9.3); 7.428(9.4); 7.421(10.9); 7.405(9.6); 7.399(10.8); 7.314(5.3); 7.308(5.4); 7.293(9.2); 7.288(9.1); 7.273(5.8); 7.262(78.3); 6.998(0.4); 5.391(5.8); 5.376(6.2); 4.988(1.8); 4.970(6.0); 4.953(8.4);
4.935(6.1); 4.917(1.9); 4.373(2.8); 4.354(7.4); 4.335(7.4); 4.315(2.7); 2.713(1.8); 2.696(4.6); 2.678(5.6); 2.663(5.5); 2.646(5.3); 2.629(2.6); 2.484(1.4); 2.467(2.2); 2.453(4.9); 2.430(9.2); 2.412(16.0);
2.394(10.3); 2.373(3.6); 2.361(2.6); 2.341(1.0); 2.245(0.4); 2.228(0.6); 2.199(3.2); 2.183(5.1); 2.168(5.7); 2.155(5.7); 2.143(4.8); 2.128(3.7); 2.112(1.8); 2.044(0.7); 1.555(81.6); 1.260(0.7); 0.147(0.4);
0.000(82.1); −0.149(0.4)
Example 491: ¹H-NMR(299.9 MHz, cdcl3):
δ = 8.762(0.6); 8.751(0.7); 8.747(0.6); 8.483(0.7); 8.478(0.7); 8.455(0.6); 8.450(0.7); 8.379(0.5); 8.367(0.6); 8.348(0.5); 7.861(0.4); 7.842(0.5); 7.831(0.5); 7.666(5.9); 7.648(6.2); 7.637(7.2); 7.619(6.9);
7.605(1.7); 7.577(1.6); 7.564(1.2); 7.558(1.0); 7.546(1.4); 7.495(0.9); 7.480(10.3); 7.476(10.6); 7.454(13.0); 7.450(13.5); 7.425(7.8); 7.416(9.0); 7.395(7.9); 7.386(9.0); 7.369(7.8); 7.362(9.5); 7.343(13.2);
7.337(16.0); 7.330(5.6); 7.320(4.4); 7.296(12.1); 7.292(14.0); 7.272(18.5); 7.268(20.6); 7.259(261.1); 7.247(10.2); 7.242(8.5); 7.222(10.5); 7.215(10.0); 7.196(10.3); 7.190(9.9); 7.171(4.6); 7.165(4.2);
7.014(0.3); 6.907(1.4); 5.781(3.2); 5.762(3.4); 5.047(1.3); 5.023(4.4); 5.000(6.0); 4.976(4.6); 4.951(1.4); 4.381(1.9); 4.358(4.9); 4.331(4.8); 4.305(1.7); 4.133(0.6); 4.118(0.5); 4.109(0.5); 2.727(1.2);
2.722(1.5); 2.701(3.5); 2.674(4.1); 2.669(3.1); 2.662(3.6); 2.658(3.8); 2.653(4.2); 2.649(2.4); 2.638(4.0); 2.633(4.2); 2.628(2.3); 2.613(2.1); 2.609(2.1); 2.490(0.7); 2.469(1.1); 2.449(4.1); 2.433(5.4);
2.429(5.9); 2.409(10.5); 2.396(8.3); 2.382(8.3); 2.376(5.5); 2.358(3.2); 2.354(2.9); 2.341(1.5); 2.314(0.6); 2.225(3.1); 2.205(4.7); 2.191(2.8); 2.185(4.5); 2.168(4.2); 2.164(4.0); 2.152(3.1); 2.144(2.3);
2.132(2.7); 2.109(1.4); 2.052(0.3); 2.043(1.8); 1.538(18.1); 1.454(0.4); 1.282(0.6); 1.258(1.5); 1.234(0.6); 0.833(0.5); 0.194(1.2); 0.066(0.5); 0.060(0.4); 0.010(10.4); −0.001(286.5); −0.012(9.7); −0.200(1.2)
Example 492: ¹H-NMR(299.9 MHz, cdcl3):
δ = 7.790(3.6); 7.772(3.9); 7.762(4.3); 7.744(3.9); 7.606(0.5); 7.400(1.5); 7.379(3.0); 7.372(3.0); 7.358(2.6); 7.351(6.5); 7.343(5.4); 7.332(8.5); 7.323(5.5); 7.311(5.9); 7.302(8.7); 7.290(6.3); 7.280(4.7);
7.260(84.6); 6.960(2.6); 6.952(9.6); 6.925(16.0); 6.897(7.8); 6.889(1.9); 6.186(2.2); 6.167(2.2); 4.977(1.0); 4.949(3.2); 4.919(4.5); 4.890(3.2); 4.859(0.9); 3.732(1.4); 3.702(3.7); 3.670(3.7); 3.640(1.4);
2.525(1.5); 2.493(3.5); 2.465(3.9); 2.435(1.9); 2.349(1.4); 2.318(3.7); 2.287(3.8); 2.256(1.6); 2.044(0.4); 2.016(1.5); 1.981(3.6); 1.950(5.3); 1.920(3.2); 1.916(3.2); 1.885(1.7); 1.835(1.8); 1.800(3.7);
1.774(4.0); 1.766(3.5); 1.738(3.0); 1.703(0.9); 1.538(33.0); 1.256(1.1); 0.196(0.5); 0.070(7.3); 0.000(93.8); −0.011(3.4); −0.198(0.4)
Example 493: ¹H-NMR(299.9 MHz, cdcl3):
δ = 13.266(0.4); 8.785(0.8); 8.769(0.8); 8.481(0.8); 8.454(0.7); 7.709(0.4); 7.689(0.7); 7.660(0.6); 7.642(4.1); 7.624(4.5); 7.613(5.1); 7.596(4.7); 7.501(0.7); 7.485(0.8); 7.472(0.8); 7.443(5.1); 7.434(5.5);
7.413(5.0); 7.404(5.4); 7.329(4.3); 7.318(3.4); 7.300(7.9); 7.290(6.4); 7.280(8.2); 7.262(81.9); 7.260(99.8); 7.231(1.8); 7.183(0.9); 7.153(1.4); 7.125(0.9); 6.962(0.4); 6.932(0.6); 6.908(0.8); 6.859(9.3);
6.832(16.0); 6.805(7.6); 5.527(2.4); 5.093(1.2); 5.067(3.6); 5.042(4.9); 5.018(3.7); 4.991(1.0); 4.369(1.5); 4.345(3.7); 4.320(3.6); 4.296(1.2); 2.720(1.0); 2.695(2.7); 2.672(3.3); 2.656(3.1); 2.632(3.1);
2.607(1.3); 2.469(0.3); 2.421(5.7); 2.395(11.7); 2.368(13.4); 2.344(5.5); 2.208(2.0); 2.185(2.8); 2.168(2.3); 2.150(2.8); 2.140(2.8); 2.129(1.5); 2.117(2.3); 2.089(1.0); 1.709(0.5); 1.548(24.6); 1.285(0.4);
1.258(1.1); 0.853(0.4); 0.197(0.7); 0.002(94.3); −0.001(111.7); −0.011(3.7); −0.198(0.6)
Example 494: ¹H-NMR(299.9 MHz, cdcl3):
δ = 7.794(6.7); 7.775(7.2); 7.765(7.9); 7.747(7.4); 7.690(8.8); 7.665(11.6); 7.628(0.7); 7.596(3.2); 7.574(9.3); 7.551(15.5); 7.546(16.0); 7.518(9.5); 7.498(15.3); 7.471(7.0); 7.430(0.4); 7.348(7.3);
7.339(11.0); 7.319(10.7); 7.310(13.3); 7.295(10.4); 7.285(7.1); 7.261(102.9); 7.260(104.4); 7.224(0.6); 7.200(0.3); 7.183(0.3); 6.909(0.6); 5.996(4.6); 5.969(4.7); 5.917(0.4); 4.984(1.8); 4.954(6.0);
4.925(8.5); 4.895(6.0); 4.865(1.7); 3.980(0.4); 3.707(2.5); 3.675(6.8); 3.643(7.0); 3.614(2.6); 2.512(2.7); 2.482(6.4); 2.453(7.0); 2.424(3.6); 2.341(2.7); 2.310(6.6); 2.279(6.9); 2.249(2.9); 2.075(0.4);
2.044(0.7); 1.991(2.5); 1.956(6.5); 1.924(9.1); 1.889(5.9); 1.860(3.0); 1.815(3.4); 1.781(6.9); 1.754(7.0); 1.747(6.4); 1.719(5.6); 1.685(1.8); 1.665(0.3); 1.545(68.1); 1.414(0.3); 1.256(2.6); 0.881(0.3);
0.850(0.4); 0.829(0.4); 0.196(0.7); 0.068(0.7); 0.001(119.0); 0.000(117.1); −0.009(5.9); −0.199(0.6)
Example 495: ¹H-NMR(299.9 MHz, cdcl3):
δ = 7.719(0.4); 7.696(0.4); 7.610(9.2); 7.599(9.0); 7.585(15.1); 7.569(7.2); 7.520(2.5); 7.498(8.9); 7.477(16.0); 7.457(7.3); 7.429(7.8); 7.420(6.8); 7.399(6.5); 7.390(6.5); 7.307(4.3); 7.299(4.2); 7.264(79.8);
7.260(104.6); 7.245(11.8); 7.220(6.9); 7.153(0.4); 7.128(0.4); 6.908(0.5); 5.368(3.7); 5.352(3.8); 5.012(1.4); 4.987(4.2); 4.965(5.7); 4.941(4.3); 4.916(1.4); 4.373(2.0); 4.348(5.0); 4.323(5.0); 4.299(1.8);
2.719(1.3); 2.693(3.3); 2.670(3.9); 2.654(3.7); 2.650(3.9); 2.645(3.4); 2.630(4.2); 2.605(2.1); 2.482(0.6); 2.441(3.6); 2.423(6.2); 2.400(9.5); 2.387(7.5); 2.376(7.6); 2.349(2.9); 2.336(1.3); 2.308(0.6);
2.200(2.6); 2.179(4.0); 2.159(4.0); 2.144(3.9); 2.126(3.0); 2.106(2.3); 2.085(1.1); 1.546(87.1); 1.542(114.1); 1.303(0.4); 1.256(1.2); 0.880(0.3); 0.851(0.4); 0.829(0.4); 0.195(0.6); 0.071(0.5); 0.004(92.2);
0.000(119.9); −0.199(0.5)
Example 496: ¹H-NMR(299.9 MHz, cdcl3):
δ = 7.782(9.5); 7.756(12.6); 7.684(8.2); 7.661(10.7); 7.656(10.5); 7.629(11.9); 7.602(16.0); 7.567(11.5); 7.542(15.5); 7.537(15.2); 7.509(9.6); 7.495(13.2); 7.490(12.3); 7.469(6.2); 7.359(6.4); 7.335(10.4);
7.308(4.7); 7.259(81.8); 7.211(0.5); 7.192(0.4); 6.908(0.4); 6.073(0.3); 6.003(4.6); 5.977(4.7); 4.995(1.8); 4.965(5.7); 4.937(8.1); 4.908(5.8); 4.878(1.8); 3.763(2.4); 3.731(6.6); 3.700(6.8); 3.669(2.5);
2.531(2.5); 2.503(6.4); 2.474(7.5); 2.445(4.6); 2.399(0.4); 2.348(3.0); 2.319(7.0); 2.288(6.9); 2.257(3.0); 1.997(2.9); 1.962(6.9); 1.931(9.4); 1.901(6.1); 1.898(6.3); 1.870(3.9); 1.862(4.1); 1.837(4.5);
1.830(7.3); 1.803(7.3); 1.796(6.3); 1.769(5.6); 1.734(1.7); 1.620(0.7); 1.541(82.2); 1.256(1.0); 0.834(0.5); 0.076(0.4); 0.011(4.5); 0.000(68.7); −0.011(3.5)
Example 499: ¹H-NMR(600.1 MHz, DMSO):
δ = 9.009(10.6); 9.002(10.8); 8.812(12.5); 8.805(12.4); 8.804(12.6); 8.064(11.5); 8.063(11.9); 8.051(12.8); 8.050(12.9); 7.802(10.8); 7.794(10.8); 7.789(10.2); 7.781(9.8); 7.449(13.8); 7.447(14.0);
7.436(16.0); 7.434(15.8); 7.304(5.3); 7.303(5.4); 7.292(13.8); 7.290(13.4); 7.279(10.0); 7.278(9.4); 7.248(8.7); 7.245(9.9); 7.235(11.8); 7.232(12.8); 7.222(5.5); 7.220(5.4); 7.158(13.6); 7.156(13.5);
7.146(11.7); 7.143(11.0); 4.096(0.3); 3.339(456.4); 3.312(0.3); 3.176(1.3); 3.167(1.4); 3.157(2.7); 3.149(6.6); 3.143(8.6); 3.136(8.7); 3.130(6.9); 3.122(2.9); 2.618(0.4); 2.615(0.5); 2.612(0.4); 2.524(0.8);
2.521(1.0); 2.518(1.0); 2.506(62.5); 2.503(86.1); 2.500(63.1); 2.390(0.4); 2.387(0.5); 2.384(0.4); 2.340(4.4); 2.334(4.7); 2.329(5.5); 2.323(9.1); 2.318(5.4); 2.313(5.1); 2.307(4.8); 1.338(4.5); 1.329(8.8);
1.321(9.0); 1.313(8.5); 1.305(6.5); 1.297(6.9); 1.286(10.7); 1.284(9.4); 1.276(6.8); 1.273(10.4); 1.263(5.5); 1.245(1.6); 0.000(2.9)
Example 500: ¹H-NMR(600.1 MHz, DMSO):
δ = 8.993(11.9); 8.985(12.1); 8.815(13.5); 8.814(14.2); 8.808(14.0); 8.806(14.0); 8.043(12.9); 8.041(13.3); 8.030(14.6); 8.028(14.6); 7.808(12.7); 7.800(12.7); 7.795(11.9); 7.787(11.4); 7.702(14.6);
7.689(16.0); 7.624(6.8); 7.612(14.6); 7.599(8.3); 7.424(8.6); 7.412(14.9); 7.399(7.0); 7.300(15.7); 7.286(14.6); 3.623(0.4); 3.616(0.4); 3.612(0.3); 3.605(0.3); 3.514(5.6); 3.289(2.7); 3.281(6.6); 3.275(8.2);
3.268(8.2); 3.262(6.6); 3.253(2.9); 3.151(0.4); 3.144(0.4); 3.139(0.4); 3.132(0.5); 3.026(0.7); 3.019(1.1); 3.015(1.9); 3.008(1.9); 3.004(1.1); 2.997(0.7); 2.618(0.5); 2.615(0.7); 2.612(0.5); 2.524(1.0);
2.521(1.3); 2.518(1.3); 2.509(37.9); 2.506(81.4); 2.503(112.1); 2.500(80.9); 2.497(37.3); 2.390(0.5); 2.387(0.7); 2.384(0.5); 2.273(6.3); 2.272(6.3); 1.912(0.7); 1.742(1.1); 1.736(1.1); 1.731(2.8); 1.726(1.1);
1.720(1.1); 1.405(3.8); 1.395(10.7); 1.382(11.7); 1.377(9.3); 1.369(12.6); 1.361(9.5); 1.353(10.7); 1.344(4.1); 1.273(3.2); 1.262(3.4); 1.258(4.1); 1.247(5.0); 1.234(1.0); 0.000(6.3)
Example 501: ¹H-NMR(299.9 MHz, cdcl3):
δ = 8.651(1.0); 8.447(0.6); 8.420(0.5); 7.967(0.6); 7.958(0.5); 7.948(0.4); 7.943(0.5); 7.936(0.7); 7.907(0.7); 7.817(4.3); 7.799(4.6); 7.788(5.0); 7.770(4.7); 7.722(0.6); 7.700(0.6); 7.692(0.8); 7.643(0.3);
7.605(1.0); 7.577(7.2); 7.573(7.1); 7.550(8.8); 7.547(8.7); 7.507(6.3); 7.501(6.7); 7.482(8.5); 7.476(8.8); 7.449(0.6); 7.434(0.7); 7.419(0.9); 7.407(1.4); 7.400(1.6); 7.387(2.0); 7.375(1.7); 7.362(4.6);
7.358(4.7); 7.333(16.0); 7.312(12.0); 7.303(9.6); 7.290(8.1); 7.283(10.4); 7.277(10.4); 7.259(118.2); 7.232(3.3); 7.226(2.9); 7.190(0.5); 6.908(0.6); 6.271(0.6); 6.230(2.8); 6.202(2.8); 5.122(0.3); 5.002(1.3);
4.972(4.0); 4.944(5.5); 4.914(3.9); 4.884(1.1); 4.180(0.5); 4.167(0.5); 4.156(0.6); 4.143(0.6); 3.749(1.6); 3.716(4.3); 3.686(4.2); 3.654(1.7); 3.613(0.5); 3.599(0.4); 3.524(0.4); 3.509(0.4); 2.515(1.9);
2.487(4.4); 2.458(4.5); 2.429(2.3); 2.409(0.4); 2.385(0.4); 2.355(1.7); 2.324(4.3); 2.292(4.5); 2.262(2.0); 2.066(0.3); 2.052(0.4); 2.035(1.9); 2.002(8.5); 1.969(6.3); 1.939(3.9); 1.934(3.6); 1.904(2.0);
1.860(0.3); 1.820(2.2); 1.785(4.8); 1.758(4.9); 1.750(4.4); 1.723(3.9); 1.687(1.5); 1.613(0.8); 1.590(0.9); 1.579(0.9); 1.563(1.0); 1.537(0.9); 1.503(0.8); 1.424(0.5); 1.356(0.4); 1.332(0.4); 1.285(0.7);
1.255(2.8); 1.211(1.4); 1.187(2.4); 1.164(1.2); 0.880(0.3); 0.852(0.4); 0.826(0.3); 0.195(0.7); 0.076(0.5); 0.009(9.4); −0.001(126.2); −0.011(3.7); −0.199(0.5)
Example 502: ¹H-NMR(299.9 MHz, cdcl3):
δ = 7.849(5.8); 7.838(3.1); 7.822(8.4); 7.809(3.4); 7.791(2.9); 7.606(0.7); 7.440(0.4); 7.413(0.5); 7.389(1.0); 7.360(9.1); 7.346(16.0); 7.334(6.1); 7.320(3.3); 7.312(4.2); 7.304(5.6); 7.293(4.9); 7.282(3.6);
7.260(106.0); 7.126(0.5); 7.112(2.9); 7.099(2.8); 7.094(2.8); 7.086(3.2); 7.082(3.0); 7.072(2.6); 7.069(3.0); 7.055(2.3); 7.043(0.6); 7.025(0.3); 6.908(0.6); 5.987(1.8); 5.959(1.9); 5.012(0.8); 4.982(2.4);
4.953(3.3); 4.923(2.3); 4.894(0.7); 3.753(1.0); 3.722(2.6); 3.690(2.6); 3.658(1.0); 2.517(1.1); 2.488(2.6); 2.460(2.7); 2.431(1.4); 2.356(1.0); 2.324(2.5); 2.294(2.7); 2.261(1.1); 2.046(1.4); 2.011(2.7);
1.980(3.8); 1.950(2.3); 1.945(2.3); 1.915(1.1); 1.816(1.2); 1.782(2.7); 1.754(2.7); 1.747(2.6); 1.719(2.2); 1.685(0.7); 1.535(24.4); 1.282(0.3); 1.258(1.0); 0.195(0.5); 0.010(6.4); 0.000(123.1); −0.011(6.3); −0.066
(0.4); −0.199(0.6)
Example 503: ¹H-NMR(299.9 MHz, cdcl3):
δ = 7.850(0.6); 7.837(0.5); 7.823(0.8); 7.779(12.5); 7.752(13.1); 7.703(0.6); 7.658(6.8); 7.640(7.2); 7.629(8.0); 7.612(7.3); 7.570(0.4); 7.537(0.4); 7.429(8.8); 7.420(9.6); 7.399(8.4); 7.390(9.3); 7.368(2.2);
7.333(6.4); 7.323(5.6); 7.299(13.8); 7.274(22.5); 7.260(168.4); 7.194(0.7); 7.147(0.5); 7.119(0.7); 7.076(13.1); 7.051(16.0); 7.025(11.0); 7.000(5.0); 6.995(4.7); 6.953(0.4); 6.909(1.0); 5.661(0.5);
5.631(0.4); 5.409(4.2); 5.317(0.4); 5.299(1.6); 5.029(1.8); 5.003(5.1); 4.981(6.7); 4.957(4.7); 4.933(1.4); 4.384(2.4); 4.359(6.0); 4.332(5.7); 4.308(2.1); 4.134(0.4); 4.111(0.5); 3.583(0.3); 3.492(0.4);
3.461(0.4); 2.724(1.5); 2.701(4.1); 2.677(4.8); 2.661(4.5); 2.656(4.5); 2.637(4.6); 2.612(2.4); 2.595(0.5); 2.569(0.5); 2.505(1.1); 2.483(1.9); 2.464(4.4); 2.440(7.5); 2.433(7.3); 2.410(13.7); 2.386(9.9);
2.360(3.2); 2.345(1.8); 2.315(0.9); 2.255(3.3); 2.235(5.3); 2.215(5.4); 2.199(5.0); 2.183(3.7); 2.162(2.9); 2.141(1.5); 2.044(0.5); 1.614(0.6); 1.538(29.0); 1.283(0.7); 1.256(1.8); 0.877(0.4); 0.858(0.6);
0.827(0.8); 0.196(1.1); 0.137(0.6); 0.000(190.7); −0.199(0.7)
Example 504: ¹H-NMR(399.9 MHz, cdcl3):
δ = 7.800(4.3); 7.786(4.8); 7.778(5.0); 7.765(4.6); 7.727(13.8); 7.709(16.0); 7.705(12.0); 7.517(0.8); 7.506(2.8); 7.487(8.2); 7.469(6.7); 7.432(12.1); 7.413(15.9); 7.395(6.2); 7.326(5.3); 7.320(7.0);
7.304(6.0); 7.297(10.1); 7.276(6.8); 7.269(6.9); 7.258(77.0); 6.995(0.4); 6.333(2.9); 6.313(2.9); 4.950(1.2); 4.927(3.7); 4.906(5.2); 4.884(3.7); 4.862(1.1); 3.733(1.6); 3.710(4.2); 3.686(4.2); 3.663(1.6);
2.502(1.7); 2.479(4.0); 2.457(4.4); 2.435(2.1); 2.326(1.6); 2.305(4.0); 2.280(4.3); 2.257(1.8); 1.989(1.6); 1.963(3.9); 1.939(5.8); 1.916(3.8); 1.913(3.6); 1.890(1.9); 1.838(1.7); 1.812(4.1); 1.792(4.3);
1.787(4.0); 1.766(3.4); 1.740(1.0); 1.541(60.6); 1.256(1.4); 0.145(0.4); −0.001(68.8)
Example 505: ¹H-NMR(399.9 MHz, cdcl3):
δ = 7.699(2.5); 7.686(2.7); 7.678(3.0); 7.665(2.8); 7.518(0.8); 7.454(3.4); 7.447(4.2); 7.439(2.3); 7.432(4.2); 7.424(6.5); 7.410(1.8); 7.405(3.2); 7.401(2.2); 7.382(2.2); 7.375(2.1); 7.361(7.4); 7.345(16.0);
7.340(12.1); 7.335(13.3); 7.317(8.7); 7.297(2.7); 7.259(120.9); 6.995(0.8); 5.647(1.5); 5.635(1.5); 5.034(0.7); 5.015(2.1); 4.997(2.8); 4.979(2.2); 4.959(0.7); 4.381(0.8); 4.362(2.1); 4.342(2.1); 4.325(0.8);
2.709(0.5); 2.694(1.5); 2.676(1.8); 2.663(1.9); 2.659(1.7); 2.643(1.8); 2.627(0.7); 2.591(0.3); 2.429(3.4); 2.409(6.6); 2.390(8.1); 2.371(3.0); 2.178(1.0); 2.160(1.5); 2.156(1.5); 2.145(1.3); 2.132(1.6);
2.125(1.6); 2.119(1.0); 2.109(1.6); 2.089(0.7); 2.043(0.4); 1.537(109.8); 1.258(0.8); 0.861(0.3); 0.147(0.6); 0.035(0.4); 0.008(7.1); 0.000(111.8); −0.006(4.8); −0.007(4.9); −0.008(4.7); −0.149(0.5)
Example 506: ¹H-NMR(399.9 MHz, cdcl3):
δ = 7.945(0.9); 7.923(0.8); 7.666(4.1); 7.653(4.4); 7.645(4.9); 7.632(4.6); 7.587(0.4); 7.553(14.1); 7.532(16.0); 7.518(1.6); 7.512(0.9); 7.427(5.8); 7.420(6.2); 7.404(5.6); 7.397(6.0); 7.348(0.7); 7.343(0.9);
7.334(3.3); 7.327(3.3); 7.313(5.1); 7.307(4.6); 7.293(3.6); 7.280(14.7); 7.275(18.0); 7.260(148.7); 7.250(12.9); 7.245(9.4); 7.229(9.4); 7.224(7.7); 6.995(1.0); 6.043(2.6); 6.030(2.6); 5.031(1.0); 5.012(3.4);
4.995(4.6); 4.977(3.5); 4.957(1.1); 4.373(1.3); 4.354(3.5); 4.336(3.5); 4.318(1.3); 2.712(0.9); 2.709(0.9); 2.693(2.8); 2.673(2.9); 2.669(2.0); 2.663(2.8); 2.657(2.8); 2.642(2.8); 2.638(1.6); 2.627(1.4);
2.623(1.2); 2.450(0.7); 2.436(3.3); 2.431(3.1); 2.420(4.6); 2.411(9.3); 2.395(6.6); 2.391(7.0); 2.379(3.5); 2.371(2.4); 2.359(0.8); 2.340(0.4); 2.195(1.7); 2.177(2.6); 2.170(1.9); 2.162(2.5); 2.152(2.6);
2.149(2.7); 2.141(2.0); 2.130(1.6); 2.123(1.8); 2.106(1.0); 1.657(0.3); 1.544(6.3); 1.278(0.4); 1.256(1.2); 0.147(0.5); 0.070(0.5); 0.032(0.3); 0.024(0.4); 0.008(6.2); 0.000(136.4); −0.007(5.9); −0.008(6.6); −0.028
(0.5); −0.149(0.4)
Example 507: ¹H-NMR(399.9 MHz, cdcl3):
δ = 7.780(3.4); 7.766(3.8); 7.759(4.1); 7.745(3.6); 7.710(0.6); 7.665(13.4); 7.644(15.3); 7.584(0.4); 7.565(0.4); 7.518(0.8); 7.447(0.4); 7.400(16.0); 7.379(13.2); 7.354(0.9); 7.330(4.3); 7.323(5.5);
7.307(5.0); 7.301(8.2); 7.279(6.3); 7.259(126.2); 6.995(0.7); 6.276(2.7); 6.257(2.6); 4.910(0.9); 4.888(2.9); 4.866(4.1); 4.844(2.8); 4.822(0.9); 3.731(1.3); 3.708(3.4); 3.684(3.4); 3.661(1.2); 2.499(1.4);
2.478(3.3); 2.456(3.6); 2.434(1.8); 2.327(1.3); 2.304(3.4); 2.280(3.6); 2.258(1.5); 2.043(0.5); 1.989(1.2); 1.963(3.1); 1.940(4.5); 1.917(3.0); 1.891(1.5); 1.853(1.5); 1.827(3.3); 1.807(3.4); 1.802(3.3);
1.781(2.6); 1.755(0.8); 1.546(56.4); 1.259(0.5); 0.146(0.5); 0.000(113.7); −0.149(0.6)
Example 508: ¹H-NMR(399.9 MHz, cdcl3):
δ = 8.021(0.9); 8.000(1.0); 7.689(5.2); 7.676(5.7); 7.667(6.2); 7.654(5.6); 7.519(1.8); 7.451(7.1); 7.444(7.9); 7.435(2.4); 7.429(6.9); 7.422(7.6); 7.401(1.0); 7.394(1.1); 7.381(4.5); 7.374(3.9); 7.361(6.4);
7.354(5.7); 7.340(4.1); 7.333(3.7); 7.314(4.1); 7.290(99.5); 7.260(278.8); 7.235(0.5); 7.188(0.5); 6.995(1.4); 5.585(3.2); 5.567(3.2); 5.016(1.4); 4.997(4.2); 4.979(5.6); 4.961(4.2); 4.941(1.4); 4.373(1.8);
4.357(4.2); 4.337(4.2); 4.320(1.5); 4.131(0.3); 4.113(0.4); 2.706(1.2); 2.688(3.1); 2.673(3.9); 2.660(3.9); 2.656(3.6); 2.640(3.7); 2.624(1.5); 2.429(6.9); 2.410(13.2); 2.389(16.0); 2.371(6.6); 2.174(2.0);
2.156(2.8); 2.151(3.4); 2.139(2.6); 2.136(3.1); 2.128(3.4); 2.120(3.3); 2.115(2.0); 2.104(3.2); 2.084(1.2); 2.071(0.4); 2.043(1.8); 1.628(0.4); 1.536(56.0); 1.277(0.8); 1.259(1.9); 1.241(0.7); 0.881(0.3);
0.862(0.6); 0.844(0.5); 0.147(1.0); 0.008(14.1); 0.000(258.3); −0.008(12.6); −0.024(0.9); −0.149(1.2)
Example 509: ¹H-NMR(299.9 MHz, cdcl3):
δ = 8.763(0.7); 8.752(0.8); 8.748(0.8); 8.480(0.8); 8.457(0.8); 8.453(0.7); 8.383(0.6); 8.356(0.8); 7.678(6.1); 7.660(6.9); 7.649(7.5); 7.632(7.1); 7.606(1.7); 7.575(6.1); 7.569(12.0); 7.561(4.0); 7.554(5.3);
7.549(9.7); 7.542(10.6); 7.533(1.1); 7.513(0.8); 7.497(1.2); 7.482(1.2); 7.468(1.0); 7.454(1.2); 7.431(8.0); 7.422(9.2); 7.401(8.0); 7.392(9.4); 7.337(5.0); 7.328(4.5); 7.309(8.5); 7.301(7.3); 7.288(14.4);
7.274(25.1); 7.267(57.2); 7.260(219.4); 7.252(12.7); 7.244(15.0); 7.236(6.8); 7.224(3.4); 7.215(3.1); 7.191(0.5); 6.908(1.1); 6.054(3.3); 6.034(3.2); 5.976(0.5); 5.067(1.6); 5.042(4.8); 5.018(6.6); 4.993(5.1);
4.968(1.6); 4.386(1.9); 4.362(5.0); 4.336(5.0); 4.309(1.7); 2.725(1.3); 2.704(4.1); 2.699(2.9); 2.683(3.7); 2.678(4.5); 2.672(2.9); 2.665(3.8); 2.657(4.0); 2.652(2.3); 2.641(3.9); 2.637(4.1); 2.632(2.0);
2.616(2.2); 2.612(2.0); 2.539(0.3); 2.486(0.4); 2.462(1.0); 2.444(5.4); 2.439(5.0); 2.423(6.8); 2.412(16.0); 2.402(4.5); 2.388(12.4); 2.369(4.9); 2.360(3.6); 2.344(1.0); 2.318(0.6); 2.216(3.0); 2.194(4.4);
2.182(3.1); 2.174(4.0); 2.160(4.2); 2.157(4.3); 2.147(2.8); 2.128(2.3); 2.122(2.5); 2.098(1.3); 2.073(0.4); 1.674(0.4); 1.544(25.0); 1.491(1.0); 1.454(0.5); 1.371(0.4); 1.285(0.7); 1.255(1.6); 0.902(0.5);
0.879(0.6); 0.858(0.7); 0.810(0.3); 0.196(1.1); 0.066(0.6); 0.010(11.5); 0.000(230.0); −0.011(7.5); −0.199(1.0)
Example 510: ¹H-NMR(299.9 MHz, cdcl3):
δ = 7.787(3.9); 7.770(4.0); 7.759(4.6); 7.741(4.3); 7.613(12.9); 7.585(15.3); 7.558(0.6); 7.530(0.6); 7.407(10.1); 7.401(12.0); 7.341(4.2); 7.332(6.3); 7.309(14.0); 7.302(16.0); 7.288(6.4); 7.281(12.0);
7.274(11.0); 7.268(5.9); 7.259(166.2); 7.242(0.9); 7.241(0.6); 7.239(0.5); 7.236(0.5); 7.234(0.5); 7.227(0.5); 7.220(0.4); 6.908(1.0); 6.471(2.6); 6.445(2.5); 4.954(1.0); 4.926(3.5); 4.897(5.1); 4.868(3.6);
4.838(1.1); 3.747(1.4); 3.716(3.9); 3.684(3.9); 3.654(1.5); 2.518(1.6); 2.487(4.1); 2.460(4.3); 2.428(2.2); 2.388(0.5); 2.355(1.6); 2.323(4.1); 2.291(4.3); 2.263(1.8); 2.170(1.6); 2.053(0.4); 2.027(2.0);
1.992(4.6); 1.961(6.2); 1.931(3.7); 1.926(3.7); 1.896(2.2); 1.842(2.1); 1.815(2.4); 1.807(4.4); 1.780(4.4); 1.773(4.1); 1.746(3.6); 1.711(1.2); 1.596(0.4); 1.532(71.7); 1.301(1.2); 1.285(1.1); 1.256(3.9);
0.890(0.5); 0.881(0.7); 0.858(0.6); 0.196(0.8); 0.067(0.4); 0.055(0.4); 0.039(0.6); 0.025(1.1); 0.020(1.7); 0.019(1.9); 0.011(7.8); 0.009(6.5); 0.000(173.8); −0.011(6.7); −0.198(0.6)
Example 511: ¹H-NMR(299.9 MHz, cdcl3):
δ = 7.699(4.5); 7.682(4.8); 7.671(5.4); 7.653(5.0); 7.605(1.3); 7.467(5.2); 7.458(6.4); 7.437(5.2); 7.428(6.2); 7.412(3.9); 7.404(7.0); 7.401(7.2); 7.394(3.8); 7.386(6.1); 7.379(10.3); 7.375(10.9); 7.348(3.3);
7.333(7.8); 7.327(11.4); 7.321(6.7); 7.298(1.1); 7.279(7.9); 7.259(196.0); 7.227(7.8); 7.194(7.6); 7.190(11.2); 7.185(6.9); 7.168(3.8); 7.164(5.5); 7.159(3.3); 6.908(1.1); 5.597(2.7); 5.573(2.8); 5.032(1.2);
5.006(3.6); 4.982(4.8); 4.958(3.7); 4.932(1.2); 4.391(1.5); 4.367(3.8); 4.344(3.7); 4.318(1.5); 4.134(0.3); 3.500(0.5); 3.484(0.4); 2.728(0.9); 2.723(1.0); 2.703(2.7); 2.677(3.4); 2.662(3.6); 2.656(3.2);
2.636(3.4); 2.614(1.7); 2.610(1.5); 2.591(0.4); 2.445(6.7); 2.417(12.7); 2.391(16.0); 2.367(6.0); 2.308(0.5); 2.186(2.1); 2.169(3.0); 2.162(3.1); 2.143(2.5); 2.126(3.2); 2.115(3.1); 2.108(1.8); 2.095(2.9);
2.068(1.2); 2.043(1.1); 2.004(0.4); 1.714(0.4); 1.703(0.4); 1.599(0.4); 1.532(103.4); 1.416(0.3); 1.394(0.3); 1.334(0.8); 1.303(1.3); 1.285(1.5); 1.256(4.5); 0.881(0.8); 0.860(0.9); 0.196(0.8); 0.011(9.0);
0.000(210.8); −0.011(10.0); −0.034(0.8); −0.044(0.6); −0.052(0.5); −0.059(0.5); −0.070(0.5); −0.134(0.3); −0.199(0.9)
Example 512: ¹H-NMR(299.9 MHz, cdcl3):
δ = 7.810(4.2); 7.792(4.4); 7.782(4.9); 7.764(4.4); 7.641(5.1); 7.639(6.1); 7.636(5.7); 7.632(5.5); 7.617(9.0); 7.609(7.6); 7.574(0.4); 7.551(0.4); 7.499(0.4); 7.472(0.4); 7.399(1.9); 7.393(4.0); 7.377(7.2);
7.373(10.5); 7.366(16.0); 7.350(11.4); 7.343(12.0); 7.331(8.3); 7.325(14.3); 7.317(14.4); 7.310(7.0); 7.301(15.4); 7.294(10.5); 7.289(7.7); 7.278(7.5); 7.271(5.3); 7.269(4.4); 7.260(135.6); 7.240(0.9);
7.235(0.5); 7.231(0.4); 7.226(0.4); 7.180(0.3); 6.909(0.7); 6.462(2.5); 6.435(2.6); 4.994(1.1); 4.964(3.8); 4.935(5.3); 4.906(3.8); 4.876(1.1); 3.748(1.5); 3.716(4.0); 3.685(4.1); 3.655(1.5); 2.520(1.7);
2.491(4.4); 2.463(4.4); 2.458(4.2); 2.431(2.1); 2.353(1.6); 2.323(4.1); 2.293(4.4); 2.261(1.9); 2.044(1.2); 2.030(2.1); 1.999(3.4); 1.994(5.0); 1.963(6.4); 1.934(3.9); 1.928(3.8); 1.899(2.2); 1.829(2.1);
1.802(2.4); 1.795(4.6); 1.768(4.6); 1.760(4.4); 1.733(3.6); 1.697(1.1); 1.543(16.1); 1.440(1.2); 1.416(0.5); 1.294(0.7); 1.283(0.8); 1.256(3.3); 1.235(0.4); 1.161(0.9); 0.880(0.4); 0.855(0.4); 0.196(0.6);
0.067(0.4); 0.027(1.0); 0.022(1.3); 0.017(2.1); 0.011(6.4); 0.009(5.0); 0.000(144.7); −0.011(5.6); −0.199(0.6)
Example 514: ¹H-NMR(299.9 MHz, dmso):
δ = 8.570(0.4); 8.547(0.4); 8.423(5.5); 8.400(5.5); 7.821(0.9); 7.795(0.9); 7.740(0.8); 7.715(0.9); 7.530(12.2); 7.507(16.0); 7.456(3.2); 7.432(7.5); 7.408(6.4); 7.369(12.0); 7.344(14.1); 7.321(5.6);
7.240(1.5); 7.219(3.2); 7.214(3.1); 7.191(5.5); 7.164(3.9); 7.142(1.8); 7.059(1.0); 7.030(1.3); 7.001(0.9); 6.955(7.8); 6.927(12.9); 6.899(6.1); 6.589(0.4); 4.866(1.1); 4.845(3.4); 4.821(4.8); 4.796(3.3);
4.773(1.0); 4.216(2.3); 4.189(5.1); 4.162(5.2); 4.135(2.0); 3.462(0.4); 3.419(0.4); 3.315(103.2); 2.724(0.4); 2.668(1.2); 2.643(2.7); 2.616(4.0); 2.586(3.8); 2.559(2.4); 2.506(37.2); 2.501(45.1); 2.455(8.8);
2.427(8.1); 2.405(4.8); 2.372(2.2); 2.341(3.5); 2.315(3.7); 2.284(2.3); 1.801(0.4); 1.676(0.4); 1.552(0.6); 1.235(2.3); 0.796(0.9); 0.772(1.6); 0.747(0.8); 0.000(18.5)
Example 515: ¹H-NMR(299.9 MHz, cdcl3):
δ = 8.464(0.3); 8.397(9.7); 8.392(10.2); 8.382(10.3); 8.377(10.1); 8.073(10.6); 8.069(10.2); 8.048(11.3); 8.043(10.6); 7.701(7.7); 7.683(8.6); 7.672(9.7); 7.655(9.1); 7.611(0.5); 7.444(8.5); 7.435(10.2);
7.414(8.6); 7.405(10.2); 7.365(5.6); 7.357(5.0); 7.338(9.2); 7.309(15.5); 7.293(12.3); 7.283(12.0); 7.265(67.2); 6.379(5.0); 6.360(5.1); 5.056(2.1); 5.031(6.4); 5.007(9.1); 4.983(6.6); 4.958(2.1); 4.402(2.7);
4.377(7.2); 4.352(7.0); 4.326(2.6); 2.804(0.8); 2.739(1.8); 2.716(4.8); 2.691(5.9); 2.671(5.7); 2.651(5.5); 2.627(2.9); 2.514(0.9); 2.492(1.6); 2.472(6.3); 2.461(7.3); 2.451(8.7); 2.436(16.0); 2.418(12.2);
2.409(12.8); 2.397(6.6); 2.382(4.5); 2.368(1.7); 2.341(0.8); 2.229(3.7); 2.207(5.8); 2.188(5.5); 2.172(6.1); 2.157(4.0); 2.144(3.3); 2.136(3.6); 2.112(1.8); 1.571(21.9); 1.299(0.3); 1.257(0.7); 0.885(0.3);
0.196(0.4); 0.002(65.4); −0.198(0.3)
Example 516: ¹H-NMR(299.9 MHz, cdcl3):
δ = 8.452(14.1); 8.445(16.0); 8.290(16.0); 8.282(15.5); 7.694(6.2); 7.676(6.8); 7.666(7.7); 7.648(7.1); 7.611(0.4); 7.403(6.4); 7.394(8.2); 7.373(6.6); 7.364(8.4); 7.351(5.2); 7.342(4.3); 7.323(7.9);
7.296(5.3); 7.287(5.3); 7.264(32.5); 7.127(0.4); 5.017(1.6); 4.991(4.8); 4.968(6.6); 4.943(5.0); 4.917(1.6); 4.386(2.2); 4.361(5.7); 4.337(5.6); 4.312(2.1); 2.804(12.4); 2.724(1.4); 2.700(3.7); 2.675(4.7);
2.655(4.6); 2.635(4.5); 2.611(2.3); 2.511(0.8); 2.489(1.5); 2.470(4.6); 2.450(7.4); 2.429(11.8); 2.416(9.3); 2.403(9.4); 2.375(3.4); 2.362(1.7); 2.335(0.9); 2.224(2.9); 2.204(4.6); 2.184(4.6); 2.168(4.9);
2.151(3.6); 2.140(2.8); 2.131(3.0); 2.108(1.6); 2.048(0.8); 1.718(0.4); 1.582(6.5); 1.399(0.3); 1.382(0.4); 1.345(0.5); 1.305(1.0); 1.283(0.9); 1.253(2.1); 1.217(0.4); 0.879(0.4); −0.001(31.0)
Example 517: ¹H-NMR(299.9 MHz, cdcl3):
δ = 10.510(0.8); 9.428(0.8); 7.757(6.0); 7.738(6.7); 7.728(7.1); 7.709(7.1); 7.680(0.9); 7.637(0.9); 7.608(2.5); 7.566(0.8); 7.494(1.1); 7.472(0.9); 7.456(1.2); 7.408(10.8); 7.378(14.9); 7.305(16.0);
7.277(12.1); 7.262(383.5); 7.225(11.0); 7.194(13.8); 7.168(7.0); 7.155(4.0); 7.125(6.5); 7.101(3.3); 7.003(1.0); 6.975(0.9); 6.911(2.2); 5.610(3.4); 5.584(3.8); 5.105(1.4); 5.078(4.3); 5.053(5.9); 5.027(4.1);
5.002(1.4); 4.981(0.9); 4.400(4.5); 4.374(4.3); 2.732(1.0); 2.707(2.6); 2.684(3.2); 2.669(4.0); 2.644(3.7); 2.617(1.8); 2.479(0.8); 2.452(1.9); 2.436(2.6); 2.410(9.8); 2.381(9.8); 2.369(4.4); 2.358(7.3);
2.339(3.9); 2.318(2.1); 2.295(1.6); 2.206(2.2); 2.177(3.8); 2.167(2.2); 2.156(3.5); 2.144(3.7); 2.116(2.9); 2.086(1.4); 1.968(0.9); 1.547(279.0); 1.482(1.5); 1.253(2.7); 0.879(0.8); 0.855(0.9); 0.196(1.5);
0.069(4.8); 0.009(18.2); 0.000(403.7); −0.010(22.1); −0.066(2.0); −0.134(0.9); −0.200(2.2); −0.553(0.8)
Example 518: ¹H-NMR(299.9 MHz, cdcl3):
δ = 13.117(0.7); 8.267(12.3); 8.262(13.2); 8.252(12.9); 8.247(13.0); 8.178(0.7); 7.816(0.7); 7.743(11.7); 7.738(12.2); 7.715(13.5); 7.711(13.6); 7.665(7.8); 7.646(8.5); 7.636(9.2); 7.617(11.0); 7.607(6.5);
7.589(4.1); 7.536(1.0); 7.514(0.8); 7.508(0.9); 7.427(7.3); 7.419(7.5); 7.392(7.3); 7.384(7.7); 7.328(1.0); 7.291(15.0); 7.275(19.5); 7.261(455.0); 7.249(18.8); 7.193(2.5); 7.127(1.0); 7.055(4.3); 7.032(6.9);
7.009(4.1); 7.001(3.6); 6.983(1.0); 6.971(1.1); 6.949(0.7); 6.910(2.9); 5.044(1.6); 5.019(4.6); 4.997(6.3); 4.972(5.1); 4.947(1.7); 4.413(2.0); 4.384(5.4); 4.358(5.2); 4.336(2.0); 3.637(0.7); 3.573(0.7);
3.515(0.7); 2.715(1.3); 2.693(4.0); 2.667(4.7); 2.654(4.0); 2.626(4.7); 2.605(2.1); 2.462(1.0); 2.440(5.8); 2.419(7.5); 2.411(16.0); 2.398(5.1); 2.386(15.4); 2.366(5.4); 2.315(1.0); 2.232(2.8); 2.212(4.3);
2.191(4.3); 2.179(4.1); 2.140(2.9); 2.119(1.4); 1.633(0.9); 1.615(1.4); 1.547(287.4); 1.479(1.8); 1.449(0.8); 1.334(0.9); 1.302(1.6); 1.254(4.0); 1.230(0.8); 1.167(0.7); 1.118(0.7); 0.883(0.8); 0.846(0.8);
0.195(2.0); 0.067(1.6); 0.011(23.1); 0.000(469.4); −0.011(19.4); −0.044(1.5); −0.068(1.7); −0.199(2.0); −1.552(0.7)
Example 519: ¹H-NMR(299.9 MHz, cdcl3):
δ = 7.790(4.5); 7.772(5.0); 7.761(5.6); 7.743(5.2); 7.715(0.5); 7.626(0.4); 7.608(1.0); 7.579(0.4); 7.540(0.5); 7.522(0.6); 7.429(0.8); 7.411(1.9); 7.401(11.9); 7.383(13.3); 7.336(5.4); 7.328(8.0); 7.306(8.8);
7.298(10.0); 7.278(7.7); 7.262(75.6); 7.179(1.3); 7.146(4.2); 7.135(16.0); 7.118(14.6); 7.018(0.8); 7.001(0.8); 6.911(0.5); 5.360(0.5); 5.343(0.6); 5.303(0.7); 5.006(1.3); 4.977(4.0); 4.948(5.9); 4.919(4.1);
4.889(1.2); 4.804(0.5); 4.785(0.5); 4.323(0.4); 4.308(0.3); 3.805(1.7); 3.774(4.7); 3.743(4.8); 3.712(1.8); 2.772(0.4); 2.755(0.4); 2.724(1.6); 2.718(1.4); 2.699(1.0); 2.676(0.8); 2.520(2.1); 2.490(5.1);
2.458(5.4); 2.431(3.1); 2.403(0.7); 2.377(2.1); 2.347(5.2); 2.314(6.1); 2.285(2.8); 2.225(0.4); 2.077(2.3); 2.042(5.8); 2.011(7.8); 1.981(4.3); 1.976(4.5); 1.945(2.1); 1.821(2.2); 1.786(5.3); 1.759(5.4);
1.752(5.2); 1.724(4.3); 1.689(1.5); 1.556(32.7); 1.506(0.6); 1.458(0.3); 1.416(0.5); 1.400(2.1); 1.367(7.9); 1.301(3.4); 1.254(8.7); 1.218(1.9); 1.167(0.7); 1.143(0.7); 0.929(0.6); 0.908(1.1); 0.895(1.2);
0.881(1.7); 0.858(1.0); 0.195(0.4); 0.000(83.2); −0.199(0.4)
Example 520: ¹H-NMR(299.9 MHz, cdcl3):
δ = 7.762(4.2); 7.744(4.4); 7.732(4.9); 7.715(4.7); 7.608(0.8); 7.444(4.7); 7.435(5.5); 7.414(4.8); 7.405(5.6); 7.383(0.5); 7.363(3.1); 7.353(2.9); 7.331(16.0); 7.314(14.7); 7.299(2.8); 7.262(121.0);
7.049(0.4); 7.018(14.0); 7.001(12.5); 6.911(0.7); 6.648(2.5); 6.629(2.7); 5.061(1.1); 5.035(3.3); 5.011(4.7); 4.987(3.5); 4.963(1.1); 4.376(1.3); 4.352(3.6); 4.329(3.4); 4.303(1.3); 2.724(0.9); 2.698(2.7);
2.678(2.8); 2.671(3.0); 2.665(2.6); 2.659(2.7); 2.651(3.0); 2.636(2.7); 2.631(2.7); 2.611(1.6); 2.507(0.4); 2.486(0.8); 2.466(3.4); 2.455(3.9); 2.445(4.6); 2.429(8.9); 2.413(6.5); 2.403(6.6); 2.392(3.5);
2.377(2.5); 2.361(1.0); 2.336(0.5); 2.252(2.0); 2.228(3.2); 2.209(2.6); 2.192(3.2); 2.163(1.8); 2.156(1.9); 2.133(1.1); 2.049(0.5); 1.552(56.6); 1.290(0.4); 1.254(0.8); 1.240(0.3); 0.195(0.6); 0.010(7.2);
0.000(137.5); −0.011(5.5); −0.199(0.7)
Example 521: ¹H-NMR(299.9 MHz, cdcl3):
δ = 8.162(0.3); 8.135(0.3); 8.075(7.8); 8.048(9.0); 7.804(4.6); 7.777(5.8); 7.758(4.8); 7.719(0.4); 7.685(3.9); 7.660(10.3); 7.635(8.1); 7.599(6.4); 7.572(8.2); 7.547(3.4); 7.473(9.9); 7.448(7.7); 7.346(12.1);
7.317(16.0); 7.291(4.1); 7.280(3.6); 7.263(88.2); 7.260(83.4); 7.025(0.6); 7.003(0.6); 6.911(0.5); 6.022(4.0); 5.993(4.1); 5.303(1.6); 4.966(1.3); 4.936(4.6); 4.907(6.6); 4.877(4.6); 4.847(1.3); 3.731(2.3);
3.699(5.4); 3.669(5.4); 3.638(1.8); 2.555(2.2); 2.524(5.2); 2.495(5.7); 2.467(2.8); 2.353(2.1); 2.323(5.2); 2.293(5.6); 2.261(2.5); 2.196(0.4); 2.048(0.7); 2.034(2.0); 2.001(5.2); 1.968(7.1); 1.936(4.7);
1.903(2.1); 1.855(3.2); 1.821(5.2); 1.794(5.4); 1.759(4.1); 1.724(1.2); 1.554(55.5); 1.502(0.3); 1.415(0.6); 1.333(0.5); 1.295(0.9); 1.254(5.7); 0.901(0.4); 0.879(0.6); 0.855(0.5); 0.833(0.3); 0.194(0.5);
0.099(0.4); 0.074(0.4); 0.000(96.6); −0.002(91.0); −0.199(0.5)
Example 522: ¹H-NMR(299.9 MHz, cdcl3):
δ = 8.215(0.5); 8.189(0.6); 7.976(7.0); 7.972(6.6); 7.949(7.9); 7.946(7.7); 7.734(0.4); 7.710(0.8); 7.685(1.0); 7.636(6.0); 7.619(6.1); 7.608(7.5); 7.590(9.2); 7.565(8.6); 7.562(8.9); 7.542(8.3); 7.535(10.3);
7.527(7.8); 7.507(7.7); 7.502(7.7); 7.481(3.3); 7.477(3.1); 7.450(6.5); 7.441(7.3); 7.420(6.3); 7.411(6.8); 7.332(3.7); 7.323(3.5); 7.305(6.0); 7.297(5.4); 7.277(4.2); 7.262(57.4); 7.193(0.7); 7.167(0.8);
7.137(8.6); 7.131(8.5); 7.113(7.3); 7.107(7.4); 5.343(3.6); 5.320(4.0); 5.024(1.5); 4.998(4.5); 4.976(5.8); 4.951(4.4); 4.926(1.3); 4.379(1.8); 4.354(4.8); 4.329(4.7); 4.302(1.8); 4.134(0.4); 2.801(3.7);
2.744(1.1); 2.723(3.4); 2.697(4.0); 2.683(3.8); 2.677(3.7); 2.657(3.8); 2.634(1.9); 2.491(0.4); 2.468(0.7); 2.447(5.7); 2.418(14.7); 2.406(4.4); 2.394(16.0); 2.372(5.7); 2.352(0.9); 2.326(0.6); 2.285(3.2);
2.265(4.4); 2.245(4.4); 2.229(4.5); 2.215(3.2); 2.194(2.9); 2.170(1.5); 2.046(0.4); 1.558(36.0); 1.254(0.5); 0.195(0.4); −0.001(62.4); −0.199(0.3)
Example 523: ¹H-NMR(299.9 MHz, cdcl3):
δ = 9.288(0.7); 8.831(0.5); 8.815(0.4); 8.659(7.0); 8.643(6.8); 8.467(10.4); 8.461(10.4); 8.367(0.4); 8.341(0.5); 7.845(5.8); 7.818(6.3); 7.706(4.8); 7.689(5.1); 7.678(6.0); 7.660(5.5); 7.608(1.4); 7.465(5.1);
7.456(6.6); 7.436(5.2); 7.427(6.4); 7.400(3.7); 7.392(3.1); 7.373(5.5); 7.346(3.3); 7.330(7.3); 7.314(7.2); 7.304(6.9); 7.288(7.2); 7.262(216.9); 7.259(183.1); 7.240(1.1); 6.911(1.2); 5.663(3.2); 5.643(3.2);
5.061(1.3); 5.035(4.2); 5.011(5.7); 4.987(4.2); 4.962(1.2); 4.399(1.6); 4.374(4.3); 4.349(4.1); 4.324(1.4); 3.494(0.4); 2.738(1.1); 2.715(3.1); 2.691(3.9); 2.676(3.6); 2.651(3.8); 2.626(1.7); 2.456(6.9);
2.429(13.8); 2.403(16.0); 2.379(6.0); 2.210(2.3); 2.189(3.5); 2.170(2.8); 2.152(3.5); 2.142(3.2); 2.132(1.8); 2.120(2.7); 2.092(1.2); 2.048(0.5); 1.776(0.4); 1.560(18.8); 1.253(0.8); 0.195(1.5); 0.000(255.5); −0.003
(208.1); −0.011(11.8); −0.200(1.1)
Example 524: ¹H-NMR(299.9 MHz, cdcl3):
δ = 8.466(6.2); 8.460(7.1); 8.450(6.8); 8.444(7.1); 8.087(7.2); 8.081(7.6); 8.062(7.9); 8.055(8.0); 8.003(0.5); 7.785(5.2); 7.767(5.5); 7.757(6.0); 7.739(5.6); 7.608(0.7); 7.349(11.4); 7.336(13.9); 7.326(12.3);
7.317(7.9); 7.310(16.0); 7.297(7.7); 7.286(5.4); 7.262(125.3); 7.229(0.7); 6.909(0.8); 6.752(3.6); 6.727(3.6); 4.963(1.4); 4.934(4.8); 4.905(6.8); 4.876(4.7); 4.846(1.4); 3.778(2.0); 3.747(5.6); 3.715(5.3);
3.685(1.9); 2.533(2.2); 2.502(5.5); 2.473(5.8); 2.442(2.7); 2.371(2.3); 2.341(5.4); 2.309(5.8); 2.276(2.4); 2.061(2.3); 2.026(5.6); 1.995(7.7); 1.959(4.7); 1.930(2.2); 1.865(2.6); 1.831(5.6); 1.804(5.8);
1.797(5.2); 1.769(4.4); 1.735(1.3); 1.553(63.5); 1.415(0.5); 1.373(1.6); 1.293(0.8); 1.253(4.2); 1.213(0.3); 0.874(0.4); 0.851(0.4); 0.824(0.4); 0.194(0.9); 0.116(0.4); 0.097(0.5); −0.001(138.1); −0.011(6.4); −0.199
(0.5)
Example 525: ¹H-NMR(299.9 MHz, cdcl3):
δ = 8.537(12.1); 8.529(15.6); 8.510(0.7); 8.480(16.0); 8.472(13.2); 7.947(3.1); 7.919(3.1); 7.797(4.4); 7.779(4.6); 7.769(5.3); 7.751(4.7); 7.608(0.7); 7.542(0.3); 7.407(0.4); 7.329(4.3); 7.321(7.8);
7.312(5.0); 7.300(6.1); 7.291(9.2); 7.284(8.7); 7.262(102.4); 7.254(13.5); 6.911(0.6); 4.966(1.2); 4.936(4.0); 4.907(5.6); 4.877(4.1); 4.847(1.1); 4.786(0.4); 3.807(1.7); 3.775(4.4); 3.743(4.4); 3.713(1.6);
2.599(0.3); 2.495(1.9); 2.466(4.6); 2.438(5.0); 2.407(2.5); 2.364(1.7); 2.335(4.6); 2.303(4.9); 2.273(2.1); 2.076(2.1); 2.040(5.0); 2.009(6.9); 1.979(4.1); 1.974(4.1); 1.944(1.9); 1.856(2.1); 1.821(5.1);
1.793(4.9); 1.786(4.7); 1.759(3.9); 1.723(1.1); 1.555(15.1); 1.364(4.5); 1.307(0.8); 1.254(1.8); 0.888(0.4); 0.196(0.7); 0.114(0.3); 0.078(0.3); 0.010(6.9); 0.000(111.5); −0.008(12.0); −0.199(0.5)
Example 526: ¹H-NMR(299.9 MHz, cdcl3):
δ = 8.933(16.0); 8.731(11.2); 8.717(11.3); 8.067(12.1); 8.041(13.0); 8.012(0.3); 7.791(8.6); 7.773(9.3); 7.763(10.2); 7.745(9.4); 7.610(0.7); 7.397(10.4); 7.381(11.0); 7.371(10.7); 7.354(10.3); 7.344(9.5);
7.335(14.1); 7.314(12.3); 7.306(15.6); 7.294(13.3); 7.285(8.4); 7.264(97.1); 6.912(0.5); 6.384(6.5); 6.356(6.6); 4.946(2.3); 4.918(7.7); 4.889(11.1); 4.859(7.8); 4.830(2.2); 3.774(3.2); 3.743(8.8); 3.712(8.8);
3.681(3.1); 2.528(3.5); 2.498(8.5); 2.469(9.5); 2.440(5.1); 2.353(3.7); 2.323(9.0); 2.292(9.2); 2.262(3.9); 2.104(0.7); 2.046(3.4); 2.011(8.5); 1.979(11.8); 1.945(7.7); 1.916(4.2); 1.893(4.4); 1.860(8.7);
1.833(9.0); 1.826(8.0); 1.798(6.7); 1.763(1.9); 1.590(13.9); 1.372(1.9); 1.284(0.5); 1.255(1.9); 0.881(0.5); 0.857(0.3); 0.195(0.6); 0.000(104.6); −0.198(0.5)
Example 527: ¹H-NMR(299.9 MHz, cdcl3):
δ = 15.156(0.9); 7.817(0.9); 7.735(8.7); 7.716(10.3); 7.707(11.2); 7.688(9.5); 7.666(1.9); 7.641(1.2); 7.603(2.4); 7.531(1.2); 7.483(0.9); 7.403(11.7); 7.367(12.1); 7.283(148.4); 7.279(149.8); 7.257(277.2);
7.253(269.9); 7.191(2.6); 7.127(6.6); 7.103(11.0); 7.080(5.7); 7.012(1.2); 6.947(0.9); 6.903(1.8); 5.576(7.1); 5.527(1.3); 5.092(1.8); 5.065(7.2); 5.040(10.4); 5.015(7.3); 4.989(2.3); 4.383(7.5); 4.361(7.4);
3.548(1.0); 3.485(0.9); 2.881(1.0); 2.724(2.5); 2.697(4.9); 2.679(6.4); 2.658(7.6); 2.637(6.7); 2.612(3.3); 2.467(1.3); 2.440(3.4); 2.428(4.7); 2.400(15.3); 2.374(16.0); 2.347(12.9); 2.328(6.5); 2.307(2.8);
2.284(1.5); 2.200(3.9); 2.171(6.5); 2.149(6.5); 2.108(4.7); 2.080(1.9); 1.876(0.9); 1.701(1.0); 1.659(0.9); 1.648(0.9); 1.612(1.9); 1.545(200.6); 1.541(195.5); 1.488(1.3); 1.478(1.7); 1.250(4.9); 1.221(1.2);
0.829(1.7); 0.807(1.3); 0.189(1.6); 0.065(19.7); 0.062(19.7); −0.004(296.1); −0.008(281.6); −0.070(1.9); −0.203(1.4)
Example 528: ¹H-NMR(299.9 MHz, cdcl3):
δ = 13.847(0.5); 10.951(0.6); 8.075(0.7); 7.811(0.6); 7.766(0.7); 7.719(7.3); 7.700(8.3); 7.690(8.4); 7.672(7.6); 7.649(1.4); 7.608(2.1); 7.568(8.1); 7.562(10.9); 7.554(5.3); 7.542(10.9); 7.535(9.6);
7.490(1.1); 7.395(9.9); 7.362(9.9); 7.336(3.3); 7.321(4.0); 7.289(12.8); 7.267(115.4); 7.262(232.3); 7.251(18.7); 7.244(15.9); 7.235(6.5); 7.223(3.4); 7.215(2.6); 7.200(0.9); 7.096(4.7); 7.070(8.1);
7.043(4.3); 7.005(0.9); 6.977(0.5); 6.911(1.0); 6.460(0.6); 6.058(4.3); 5.125(1.8); 5.099(5.4); 5.075(7.8); 5.049(5.8); 5.023(1.9); 4.879(0.7); 4.850(0.5); 4.420(2.6); 4.397(6.0); 4.373(6.1); 4.348(2.4);
3.769(0.6); 3.736(0.8); 2.817(0.6); 2.737(1.7); 2.715(4.3); 2.689(5.0); 2.674(5.5); 2.653(5.5); 2.628(2.2); 2.548(0.7); 2.516(0.9); 2.474(1.2); 2.419(5.7); 2.401(13.9); 2.373(16.0); 2.346(8.8); 2.307(1.2);
2.229(3.5); 2.199(5.4); 2.178(5.1); 2.169(4.7); 2.148(2.7); 2.139(3.9); 2.111(1.5); 2.077(0.6); 2.053(1.0); 2.016(0.8); 1.985(0.9); 1.951(0.9); 1.938(0.9); 1.915(0.8); 1.831(1.0); 1.808(1.0); 1.772(0.9);
1.731(1.0); 1.681(1.6); 1.557(40.6); 1.363(0.7); 1.343(0.8); 1.290(1.5); 1.256(2.6); 0.886(1.0); 0.859(0.9); 0.824(0.5); 0.196(1.3); 0.130(0.7); 0.075(1.5); 0.051(1.8); 0.006(81.8); 0.000(224.1); −0.199(0.9)
Example 529: ¹H-NMR(299.9 MHz, cdcl3):
δ = 16.578(0.5); 8.701(0.5); 8.506(12.2); 8.047(1.0); 7.981(11.3); 7.976(11.9); 7.686(5.1); 7.668(5.6); 7.658(6.3); 7.639(5.8); 7.608(1.6); 7.575(0.5); 7.465(3.7); 7.435(3.9); 7.419(6.9); 7.411(7.0);
7.384(6.1); 7.377(6.4); 7.262(194.6); 7.128(0.6); 7.095(3.1); 7.086(3.3); 7.064(5.5); 7.039(2.8); 6.993(0.5); 6.910(1.2); 5.052(1.5); 5.024(4.0); 5.003(5.0); 4.976(4.1); 4.950(1.4); 4.398(4.3); 4.371(4.2);
2.731(1.1); 2.711(3.1); 2.684(3.7); 2.670(3.9); 2.643(3.7); 2.618(1.7); 2.451(6.7); 2.423(13.5); 2.398(16.0); 2.373(6.6); 2.327(0.6); 2.240(2.1); 2.217(3.0); 2.198(3.3); 2.182(3.3); 2.171(3.3); 2.151(2.6);
2.124(1.3); 1.615(0.9); 1.547(155.3); 1.477(0.9); 1.449(0.5); 1.426(0.6); 1.255(1.1); 1.239(0.5); 0.196(1.0); 0.000(205.4); −0.084(0.8); −0.109(0.8); −0.134(0.5); −0.199(0.9)
Example 530: ¹H-NMR(299.9 MHz, cdcl3):
δ = 7.723(0.6); 7.705(0.6); 7.694(0.7); 7.676(0.6); 7.345(0.7); 7.336(0.9); 7.314(0.8); 7.306(1.2); 7.281(0.8); 7.276(0.6); 7.273(0.8); 7.271(0.8); 7.270(0.7); 7.268(0.8); 7.266(0.9); 7.261(14.3); 7.256(0.9);
7.254(0.7); 7.252(0.5); 7.245(0.3); 6.325(0.3); 6.304(0.3); 4.826(0.5); 4.797(0.7); 4.768(0.5); 3.701(0.5); 3.669(0.6); 2.713(16.0); 2.476(0.4); 2.470(0.5); 2.443(0.6); 2.437(0.6); 2.411(0.3); 2.322(0.5);
2.293(0.6); 1.982(0.4); 1.977(0.7); 1.947(0.9); 1.942(0.5); 1.917(0.5); 1.911(0.5); 1.883(0.3); 1.824(0.3); 1.817(0.6); 1.790(0.6); 1.782(0.6); 1.755(0.5); 1.544(2.0); 1.256(0.6); 0.011(0.5); 0.007(0.4);
0.006(0.6); 0.000(15.7); −0.008(0.5); −0.010(0.6)
Example 531: ¹H-NMR(299.9 MHz, cdcl3):
δ = 7.644(1.0); 7.627(1.1); 7.616(1.3); 7.598(1.1); 7.446(1.1); 7.437(1.3); 7.416(1.1); 7.408(1.2); 7.364(0.7); 7.356(0.7); 7.338(1.2); 7.311(0.6); 7.260(11.6); 5.767(0.7); 4.911(0.8); 4.887(1.1); 4.863(0.8);
4.360(0.4); 4.334(1.0); 4.309(0.9); 4.283(0.3); 2.699(0.4); 2.661(16.0); 2.614(0.7); 2.589(0.4); 2.450(0.7); 2.430(1.3); 2.407(1.8); 2.378(1.5); 2.354(0.5); 2.182(0.4); 2.160(0.7); 2.141(0.7); 2.124(0.8);
2.108(0.6); 2.089(0.5); 2.045(0.6); 1.543(5.5); 1.283(0.4); 1.257(1.4); 0.000(12.6)
Example 532: ¹H-NMR(400.0 MHz, DMSO):
δ = 8.949(5.7); 8.944(7.0); 8.905(7.3); 8.899(5.6); 8.839(3.0); 8.829(3.0); 7.285(1.1); 7.279(7.6); 7.274(3.1); 7.263(4.1); 7.258(16.0); 7.252(2.5); 7.229(0.7); 7.217(14.7); 7.200(2.8); 7.195(7.2); 4.038(0.4);
4.020(0.5); 3.326(78.8); 3.225(0.8); 3.213(1.6); 3.206(1.8); 3.195(3.0); 3.184(1.8); 3.176(1.6); 3.164(0.9); 2.676(0.4); 2.671(0.6); 2.667(0.4); 2.541(0.4); 2.511(33.0); 2.507(64.9); 2.502(84.9); 2.498(61.2);
2.493(29.4); 2.348(1.4); 2.329(3.4); 2.307(3.0); 2.289(1.4); 1.989(2.0); 1.401(1.6); 1.386(2.2); 1.381(2.3); 1.379(2.3); 1.364(2.7); 1.359(2.0); 1.343(1.7); 1.269(1.9); 1.253(2.9); 1.238(3.0); 1.223(1.5);
1.193(0.6); 1.175(1.1); 1.157(0.6); 0.146(0.4); 0.008(4.1); 0.000(94.1); −0.009(3.3); −0.150(0.4)
Example 533: ¹H-NMR(299.9 MHz, cdcl3):
δ = 8.465(9.4); 8.458(10.2); 8.449(9.5); 8.443(9.5); 8.140(0.5); 8.126(11.9); 8.120(11.7); 8.101(12.8); 8.094(12.1); 7.606(1.0); 7.568(7.2); 7.563(7.3); 7.542(8.4); 7.537(8.4); 7.392(0.5); 7.354(15.7);
7.352(9.1); 7.347(8.8); 7.339(14.6); 7.329(15.3); 7.325(11.2); 7.321(16.0); 7.313(14.5); 7.295(10.6); 7.291(8.7); 7.284(1.8); 7.282(1.8); 7.280(1.8); 7.277(2.5); 7.270(9.8); 7.265(11.3); 7.260(127.4);
7.252(2.7); 7.248(0.5); 7.201(5.9); 7.195(6.0); 7.186(0.6); 7.175(7.8); 7.170(7.5); 7.149(3.0); 7.145(2.8); 6.909(0.9); 6.831(2.8); 6.806(2.8); 4.900(1.3); 4.872(4.7); 4.843(6.6); 4.814(4.7); 4.783(1.4);
3.852(2.7); 3.820(6.4); 3.789(6.7); 3.758(2.4); 2.535(2.0); 2.531(2.1); 2.502(5.5); 2.496(3.1); 2.475(5.4); 2.468(5.4); 2.453(4.0); 2.448(3.2); 2.444(4.1); 2.428(4.3); 2.422(5.7); 2.419(5.5); 2.399(2.6);
2.393(6.0); 2.389(6.3); 2.384(4.0); 2.363(2.0); 2.360(2.5); 2.080(2.7); 2.050(4.6); 2.045(6.4); 2.014(9.4); 2.009(4.8); 1.984(5.4); 1.979(5.2); 1.949(2.4); 1.858(2.7); 1.830(3.9); 1.822(7.8); 1.795(6.9);
1.788(7.1); 1.761(6.1); 1.752(2.8); 1.725(2.0); 1.615(0.7); 1.600(0.4); 1.548(151.7); 1.514(0.4); 1.481(0.4); 1.256(2.5); 0.880(0.4); 0.856(0.5); 0.833(0.5); 0.195(0.4); 0.017(0.5); 0.015(0.8); 0.010(4.1);
0.007(2.1); 0.000(105.3); −0.009(4.0); −0.011(4.1); −0.016(1.2); −0.017(1.1); −0.019(0.9); −0.021(0.7); −0.022(0.7); −0.024(0.6); −0.026(0.5); −0.027(0.4); −0.029(0.4); −0.031(0.3); −0.033(0.4); −0.034(0.3); −0.036
(0.4); −0.038(0.4); −0.044(0.4); −0.199(0.4)
Example 534: ¹H-NMR(299.9 MHz, cdcl3):
δ = 8.373(11.6); 8.367(12.9); 8.358(12.7); 8.351(13.0); 8.058(14.0); 8.051(14.2); 8.032(15.1); 8.026(14.8); 7.607(0.5); 7.472(10.2); 7.447(14.8); 7.415(10.0); 7.411(11.3); 7.389(13.2); 7.385(16.0);
7.367(7.1); 7.363(7.4); 7.342(14.4); 7.338(13.3); 7.317(8.2); 7.312(7.2); 7.281(17.1); 7.265(28.1); 7.261(56.0); 7.256(23.0); 7.240(25.4); 7.236(14.6); 7.216(4.8); 7.210(4.6); 6.363(4.2); 6.348(4.2);
5.124(1.7); 5.102(5.5); 5.078(8.0); 5.054(5.7); 5.030(1.7); 4.376(3.0); 4.351(7.9); 4.325(7.9); 4.299(2.9); 4.156(0.4); 4.132(0.6); 4.108(0.5); 2.693(1.8); 2.688(2.0); 2.672(2.7); 2.667(4.2); 2.664(4.5);
2.659(3.3); 2.653(2.9); 2.648(4.8); 2.643(5.5); 2.639(5.4); 2.634(5.1); 2.628(5.2); 2.625(5.3); 2.620(5.6); 2.614(3.8); 2.609(3.8); 2.604(6.1); 2.600(5.4); 2.580(3.4); 2.575(3.2); 2.533(2.6); 2.511(3.7);
2.501(3.2); 2.492(5.0); 2.479(4.9); 2.471(6.6); 2.460(5.8); 2.450(3.7); 2.439(6.9); 2.418(3.5); 2.402(3.2); 2.397(3.3); 2.380(4.1); 2.374(8.0); 2.369(5.9); 2.363(3.0); 2.357(3.1); 2.352(6.7); 2.347(8.3);
2.340(5.2); 2.335(5.0); 2.329(3.7); 2.324(3.7); 2.319(3.5); 2.312(3.9); 2.307(4.5); 2.301(1.8); 2.284(1.7); 2.279(1.6); 2.159(3.4); 2.157(3.4); 2.138(5.8); 2.127(3.8); 2.124(4.1); 2.118(5.9); 2.105(5.8);
2.102(6.0); 2.098(5.9); 2.086(5.0); 2.079(3.3); 2.076(3.2); 2.066(4.1); 2.042(3.2); 1.578(39.0); 1.281(0.9); 1.257(2.6); 1.234(0.7); 0.010(1.9); −0.001(35.3); −0.011(1.5)
Example 535: ¹H-NMR(399.8 MHz, cdcl3):
δ = 7.577(0.6); 7.558(0.8); 7.501(7.7); 7.482(9.3); 7.409(1.8); 7.373(10.8); 7.352(16.0); 7.332(7.1); 7.298(18.1); 7.260(61.7); 7.219(9.1); 7.199(6.8); 6.996(0.4); 6.502(3.9); 6.481(3.8); 6.425(0.4);
5.239(1.5); 5.218(4.0); 5.199(4.9); 5.179(3.6); 5.156(1.2); 4.482(2.4); 4.462(5.0); 4.442(4.9); 4.421(2.1); 2.764(0.5); 2.702(4.1); 2.692(4.0); 2.682(4.0); 2.658(2.0); 2.618(1.8); 2.591(3.5); 2.575(4.6);
2.563(4.4); 2.545(2.7); 2.527(1.5); 2.443(6.5); 2.425(8.3); 2.410(5.4); 1.541(37.3); 1.254(1.1); 0.146(0.7); 0.120(0.4); −0.001(68.7)
Example 536: ¹H-NMR(399.8 MHz, cdcl3):
δ = 7.630(0.4); 7.609(0.5); 7.519(0.7); 7.469(13.6); 7.448(15.8); 7.421(0.6); 7.400(0.5); 7.383(0.6); 7.362(0.7); 7.330(16.0); 7.309(15.6); 7.303(12.6); 7.283(6.6); 7.260(74.8); 6.996(0.4); 6.414(2.2);
6.397(2.2); 5.187(0.7); 5.165(2.1); 5.148(2.8); 5.127(2.3); 5.105(0.8); 4.477(1.2); 4.455(3.0); 4.434(3.0); 4.412(1.1); 2.759(1.3); 2.750(1.4); 2.743(1.7); 2.735(2.3); 2.721(1.7); 2.713(2.2); 2.704(2.1);
2.699(2.1); 2.692(1.9); 2.680(1.9); 2.656(2.3); 2.642(1.7); 2.629(1.6); 2.611(0.7); 2.491(0.6); 2.466(1.6); 2.451(2.0); 2.443(2.4); 2.420(1.8); 2.390(0.7); 2.375(1.0); 2.358(1.5); 2.348(1.9); 2.332(2.3);
2.317(1.6); 1.541(43.7); 1.255(0.5); 0.147(0.5); 0.000(86.2); −0.150(0.4)
Example 537: ¹H-NMR(399.8 MHz, cdcl3):
δ = 8.454(0.3); 8.381(14.9); 8.377(15.4); 8.369(15.6); 8.366(15.2); 8.186(4.1); 8.165(4.1); 7.719(14.2); 7.716(14.2); 7.699(16.0); 7.695(15.2); 7.519(1.4); 7.509(0.5); 7.502(0.5); 7.486(0.4); 7.465(0.5);
7.439(8.4); 7.431(9.4); 7.424(10.3); 7.416(10.8); 7.403(1.9); 7.381(0.8); 7.361(0.5); 7.328(0.8); 7.310(1.2); 7.292(17.9); 7.279(32.9); 7.271(24.9); 7.260(237.3); 7.246(25.0); 7.234(2.4); 7.207(0.5);
7.191(0.4); 6.995(1.3); 6.272(0.3); 5.199(1.7); 5.177(4.2); 5.160(5.6); 5.138(5.0); 5.123(1.6); 5.114(1.7); 4.478(2.6); 4.457(5.8); 4.438(5.7); 4.417(2.9); 3.487(0.4); 2.759(0.6); 2.749(1.2); 2.728(2.8);
2.722(2.8); 2.715(3.9); 2.707(5.3); 2.695(5.5); 2.688(6.6); 2.671(5.2); 2.666(6.9); 2.651(6.2); 2.636(4.0); 2.617(2.5); 2.567(0.6); 2.513(0.3); 2.478(1.3); 2.458(4.8); 2.441(8.5); 2.437(8.1); 2.431(9.2);
2.424(9.4); 2.416(11.0); 2.400(3.9); 2.392(3.5); 2.369(0.9); 2.352(0.3); 1.592(0.5); 1.541(88.9); 1.254(0.9); 0.841(0.4); 0.146(1.1); 0.121(0.4); 0.050(0.9); 0.007(12.9); −0.001(246.0); −0.009(8.6); −0.150
(1.0)
Example 538: ¹H-NMR(299.9 MHz, cdcl3):
δ = 8.213(0.4); 8.186(0.4); 7.968(6.9); 7.964(6.1); 7.962(5.9); 7.942(9.0); 7.937(8.3); 7.727(0.6); 7.702(0.4); 7.627(0.4); 7.606(0.9); 7.574(0.4); 7.560(3.1); 7.555(4.0); 7.535(9.1); 7.530(10.6); 7.511(10.7);
7.506(15.2); 7.500(11.2); 7.480(8.6); 7.474(10.2); 7.455(4.7); 7.449(9.7); 7.443(11.3); 7.435(8.2); 7.422(10.4); 7.417(16.0); 7.410(10.7); 7.379(1.1); 7.343(4.4); 7.339(4.7); 7.318(9.1); 7.314(8.7);
7.293(5.0); 7.289(5.1); 7.279(1.2); 7.277(1.2); 7.275(1.2); 7.272(1.5); 7.270(1.7); 7.260(74.8); 7.249(8.4); 7.238(1.3); 7.229(7.5); 7.223(7.6); 7.203(3.0); 7.198(2.9); 7.179(0.5); 7.148(9.1); 7.142(11.1);
7.125(6.2); 7.123(6.7); 7.118(8.1); 6.909(0.5); 5.412(2.7); 5.389(3.1); 5.161(1.3); 5.137(3.9); 5.112(5.1); 5.110(5.2); 5.085(3.7); 5.061(1.1); 4.363(1.8); 4.337(4.7); 4.312(4.7); 4.286(1.7); 4.156(0.5);
4.132(0.9); 4.108(0.9); 4.084(0.4); 2.705(1.2); 2.700(1.3); 2.686(1.6); 2.680(2.9); 2.678(2.6); 2.672(2.3); 2.666(2.0); 2.661(3.3); 2.658(3.0); 2.656(3.0); 2.653(3.8); 2.647(3.6); 2.642(3.6); 2.638(3.2);
2.633(3.8); 2.628(2.4); 2.622(2.4); 2.618(3.2); 2.613(3.9); 2.608(2.1); 2.594(2.0); 2.588(1.9); 2.486(1.1); 2.465(1.8); 2.454(1.7); 2.446(3.6); 2.433(2.8); 2.426(4.3); 2.414(4.5); 2.406(3.0); 2.393(7.3);
2.373(5.8); 2.370(7.8); 2.365(4.8); 2.358(1.8); 2.353(1.9); 2.346(5.4); 2.342(6.6); 2.337(3.3); 2.329(3.2); 2.325(2.4); 2.318(3.0); 2.314(2.8); 2.306(2.4); 2.302(2.7); 2.278(1.2); 2.274(1.1); 2.210(2.4);
2.207(2.4); 2.187(4.0); 2.177(2.5); 2.174(2.6); 2.171(2.9); 2.167(3.6); 2.154(3.8); 2.151(4.1); 2.148(4.1); 2.135(2.8); 2.131(2.4); 2.127(2.4); 2.124(2.4); 2.115(2.8); 2.094(1.4); 2.092(1.4); 2.042(3.6);
1.549(73.2); 1.282(1.3); 1.258(3.4); 1.234(1.0); 0.853(0.4); 0.022(0.5); 0.020(0.5); 0.017(0.7); 0.010(2.7); 0.008(2.1); 0.000(63.3); −0.011(2.9); −0.017(0.5)
Example 539: ¹H-NMR(299.9 MHz, cdcl3):
δ = 8.967(12.0); 8.729(8.1); 8.717(8.1); 8.115(6.3); 8.108(9.1); 8.102(6.2); 8.089(6.8); 8.082(9.6); 8.076(6.2); 7.606(0.9); 7.563(9.4); 7.558(9.5); 7.537(11.1); 7.533(11.0); 7.399(7.7); 7.383(8.1); 7.373(7.8);
7.356(7.8); 7.350(9.9); 7.346(10.1); 7.324(12.1); 7.319(16.0); 7.291(12.6); 7.287(11.1); 7.279(2.8); 7.277(2.9); 7.260(113.3); 7.247(1.1); 7.245(0.7); 7.197(7.7); 7.192(7.5); 7.171(9.9); 7.166(9.5);
7.146(3.9); 7.141(3.5); 6.909(0.6); 6.494(4.5); 6.472(4.5); 4.885(1.9); 4.855(5.8); 4.826(8.3); 4.798(5.9); 4.769(1.8); 4.198(0.4); 4.175(0.4); 3.862(3.6); 3.831(8.6); 3.799(8.8); 3.768(3.5); 2.524(2.8);
2.494(6.9); 2.467(7.1); 2.435(4.0); 2.422(3.3); 2.390(7.7); 2.357(8.1); 2.327(3.3); 2.060(3.1); 2.029(6.0); 2.025(7.6); 1.994(10.6); 1.965(6.4); 1.959(6.4); 1.931(3.5); 1.874(4.1); 1.847(4.6); 1.839(8.9);
1.813(8.9); 1.805(8.2); 1.778(7.1); 1.770(3.1); 1.742(2.1); 1.570(13.0); 1.306(0.4); 1.298(0.4); 1.282(0.7); 1.273(0.6); 1.258(0.8); 0.195(0.5); 0.038(0.4); 0.033(0.4); 0.031(0.5); 0.028(0.6); 0.027(0.6);
0.025(0.7); 0.020(1.2); 0.015(2.0); 0.010(5.9); 0.008(5.5); −0.001(122.5); −0.011(5.2); −0.017(1.0); −0.019(0.7); −0.021(0.5); −0.023(0.6); −0.024(0.6); −0.031(0.4); −0.033(0.4); −0.199(0.5)
Example 540: ¹H-NMR(299.9 MHz, cdcl3):
δ = 8.215(0.6); 8.187(0.7); 8.070(0.9); 8.043(0.9); 8.024(0.4); 7.980(7.7); 7.976(6.8); 7.954(9.2); 7.949(8.8); 7.745(6.5); 7.726(6.9); 7.716(7.9); 7.697(7.0); 7.657(1.4); 7.653(1.5); 7.645(1.2); 7.628(2.1);
7.606(2.3); 7.596(2.0); 7.590(1.7); 7.577(3.9); 7.572(4.8); 7.564(2.4); 7.552(10.1); 7.548(11.3); 7.528(14.9); 7.523(13.9); 7.520(14.3); 7.501(9.5); 7.495(9.9); 7.476(4.9); 7.470(3.7); 7.447(1.1); 7.380(1.0);
7.342(7.3); 7.335(7.6); 7.309(7.2); 7.302(7.5); 7.260(163.2); 7.231(1.3); 7.205(0.9); 7.158(0.5); 7.112(10.5); 7.106(11.5); 7.088(10.6); 7.083(11.8); 7.063(6.2); 7.039(3.6); 7.031(3.1); 6.986(0.5); 6.909(1.0);
6.016(0.4); 5.990(0.4); 5.397(3.7); 5.373(4.1); 5.093(1.7); 5.067(5.1); 5.041(6.2); 5.019(4.8); 4.993(1.5); 4.873(0.4); 4.843(0.6); 4.813(0.4); 4.420(2.0); 4.395(5.1); 4.369(5.0); 4.345(1.7); 3.759(0.4);
3.726(0.5); 3.490(0.4); 3.077(0.5); 2.966(0.3); 2.843(0.4); 2.817(0.4); 2.763(1.4); 2.758(1.5); 2.737(4.2); 2.716(4.4); 2.711(5.0); 2.695(5.0); 2.685(2.5); 2.673(5.2); 2.649(2.1); 2.645(2.0); 2.577(0.3);
2.544(0.7); 2.513(0.8); 2.475(0.8); 2.447(1.7); 2.426(5.9); 2.403(14.1); 2.376(16.0); 2.351(9.5); 2.332(1.5); 2.306(0.9); 2.269(3.9); 2.249(4.3); 2.240(5.1); 2.229(4.5); 2.220(5.1); 2.211(5.1); 2.202(4.5);
2.181(3.7); 2.151(1.4); 2.023(0.7); 1.992(0.9); 1.957(0.8); 1.927(0.6); 1.894(0.5); 1.861(0.7); 1.833(0.8); 1.799(0.8); 1.744(0.4); 1.719(0.5); 1.545(19.9); 1.396(0.4); 1.376(0.4); 1.333(0.6); 1.310(0.8);
1.305(0.6); 1.282(0.7); 1.255(1.7); 1.068(0.6); 1.061(0.4); 1.044(0.4); 0.882(0.8); 0.857(1.2); 0.832(0.7); 0.195(0.9); 0.095(0.4); 0.069(0.8); 0.010(12.2); 0.000(183.6); −0.011(6.6); −0.199(0.6)
Example 541: ¹H-NMR(299.9 MHz, cdcl3):
δ = 7.861(0.4); 7.850(0.5); 7.824(0.6); 7.779(7.1); 7.752(7.4); 7.718(4.6); 7.699(5.0); 7.689(5.2); 7.670(4.7); 7.607(0.7); 7.553(0.4); 7.513(0.4); 7.374(5.7); 7.367(6.1); 7.341(6.7); 7.333(7.1); 7.310(1.3);
7.296(4.4); 7.293(4.6); 7.261(73.0); 7.246(7.0); 7.243(6.4); 7.147(0.5); 7.127(0.8); 7.074(8.1); 7.069(10.5); 7.049(13.3); 7.043(11.0); 7.024(9.6); 7.019(6.7); 6.999(4.0); 6.993(3.3); 6.909(0.4); 5.478(2.8);
5.455(3.0); 5.087(1.5); 5.061(3.9); 5.039(4.7); 5.013(3.8); 4.988(1.2); 4.415(1.6); 4.392(4.0); 4.366(3.9); 4.342(1.4); 2.742(1.1); 2.717(2.9); 2.714(3.0); 2.693(3.7); 2.677(3.9); 2.672(3.2); 2.651(3.7);
2.629(1.7); 2.472(0.5); 2.431(6.7); 2.404(13.7); 2.378(16.0); 2.354(7.0); 2.312(0.5); 2.260(2.5); 2.238(3.4); 2.228(2.8); 2.218(3.5); 2.202(3.3); 2.191(3.4); 2.171(2.7); 2.143(1.1); 2.006(0.4); 1.624(1.2);
1.552(90.2); 1.319(0.8); 1.295(1.4); 1.270(1.1); 1.255(1.5); 1.093(0.4); 1.070(0.8); 1.045(0.4); 0.906(0.5); 0.883(1.0); 0.858(0.7); 0.828(0.4); 0.072(0.5); 0.000(57.3)
Example 542: ¹H-NMR(399.8 MHz, cdcl3):
δ = 14.115(0.5); 7.838(0.8); 7.829(1.4); 7.808(1.1); 7.771(15.9); 7.751(16.0); 7.612(0.5); 7.518(4.0); 7.509(13.7); 7.489(16.0); 7.422(1.8); 7.407(2.6); 7.380(4.8); 7.360(9.9); 7.346(10.7); 7.326(8.2);
7.317(11.3); 7.288(14.0); 7.260(314.3); 7.238(11.0); 7.194(0.5); 7.068(1.3); 7.041(8.3); 7.037(9.0); 7.021(13.9); 7.018(14.3); 7.002(6.9); 6.998(7.6); 6.968(15.8); 6.964(13.7); 6.949(13.9); 6.945(11.7);
6.778(0.5); 6.268(0.5); 6.237(0.5); 6.044(5.5); 6.025(5.5); 5.245(2.3); 5.224(6.3); 5.202(7.1); 5.184(5.6); 5.163(1.9); 5.144(0.5); 4.494(3.4); 4.472(7.5); 4.453(8.1); 4.430(3.3); 4.149(0.6); 4.131(0.5);
3.714(0.5); 3.520(0.6); 3.479(0.5); 3.454(0.6); 2.766(0.8); 2.739(3.3); 2.732(4.1); 2.724(4.9); 2.717(5.7); 2.709(5.1); 2.695(6.0); 2.687(4.3); 2.674(3.0); 2.664(2.6); 2.647(0.8); 2.611(2.0); 2.586(4.4);
2.568(6.0); 2.553(6.3); 2.536(3.7); 2.521(2.3); 2.484(2.7); 2.467(5.4); 2.445(7.7); 2.437(8.4); 2.427(9.2); 2.420(12.4); 2.413(9.3); 2.404(7.4); 2.370(2.5); 2.359(1.4); 2.236(0.7); 2.202(0.5); 2.169(0.5);
2.136(1.7); 2.111(2.0); 2.071(0.5); 1.588(0.8); 1.538(166.1); 1.447(0.4); 1.310(0.5); 1.296(0.7); 1.255(1.7); 1.237(0.6); 1.231(0.5); 0.881(0.9); 0.863(0.7); 0.839(0.8); 0.146(1.6); 0.000(354.7); −0.008(21.3);
−0.047(1.4); −0.131(0.5); −0.150(1.9); −0.180(0.5)
Example 543: ¹H-NMR(399.8 MHz, cdcl3):
δ = 8.742(0.8); 8.704(1.5); 8.672(0.3); 8.637(14.9); 8.635(14.6); 8.078(4.5); 8.059(4.3); 8.023(1.6); 7.962(14.8); 7.959(14.4); 7.884(0.3); 7.520(0.6); 7.454(7.3); 7.449(6.0); 7.442(8.4); 7.431(9.2);
7.420(1.4); 7.393(0.8); 7.324(1.1); 7.301(16.0); 7.290(18.2); 7.274(11.3); 7.268(18.7); 7.261(79.9); 7.247(1.9); 7.212(0.4); 6.997(0.5); 5.211(0.4); 5.191(1.6); 5.169(3.9); 5.152(5.0); 5.130(4.5); 5.107(1.5);
4.491(2.4); 4.470(5.4); 4.450(5.5); 4.428(2.3); 4.285(0.4); 3.790(0.3); 2.774(0.8); 2.766(0.9); 2.754(1.6); 2.746(2.3); 2.740(2.7); 2.730(3.5); 2.724(4.4); 2.716(3.5); 2.702(5.9); 2.696(4.4); 2.682(5.2);
2.677(5.3); 2.660(4.6); 2.647(4.1); 2.630(2.7); 2.610(1.4); 2.576(0.6); 2.552(0.4); 2.504(1.2); 2.498(1.5); 2.476(3.4); 2.461(5.6); 2.453(5.8); 2.442(6.3); 2.434(8.7); 2.428(7.9); 2.418(5.6); 2.399(3.5);
2.392(3.0); 2.386(2.2); 2.367(1.1); 2.348(0.5); 2.329(0.8); 2.309(1.0); 2.290(0.6); 2.215(0.3); 2.189(0.5); 2.165(0.5); 2.096(0.4); 2.090(0.4); 2.068(0.7); 2.057(0.6); 2.041(0.8); 2.020(0.7); 1.995(0.6);
1.979(0.5); 1.972(0.5); 1.959(0.4); 1.930(0.4); 1.906(0.4); 1.878(0.4); 1.834(0.4); 1.810(0.5); 1.789(0.5); 1.760(0.5); 1.732(0.5); 1.624(0.8); 1.605(0.8); 1.547(39.8); 1.490(1.1); 1.450(0.6); 1.416(0.8);
1.394(0.6); 1.371(0.7); 1.303(2.2); 1.295(1.8); 1.286(1.6); 1.255(6.8); 1.091(0.3); 1.022(0.4); 1.004(0.8); 0.986(0.4); 0.896(0.7); 0.890(0.9); 0.880(1.2); 0.873(0.6); 0.863(0.6); 0.146(0.4); 0.050(0.3);
0.000(89.8); −0.008(4.0); −0.149(0.4)
Example 544: ¹H-NMR(299.9 MHz, cdcl3):
δ = 7.606(1.0); 7.584(6.0); 7.558(7.0); 7.526(0.3); 7.399(12.2); 7.382(13.4); 7.340(6.1); 7.336(6.3); 7.314(8.5); 7.309(12.5); 7.294(2.0); 7.284(9.3); 7.279(8.3); 7.260(145.7); 7.242(1.7); 7.240(1.7);
7.235(1.4); 7.203(3.1); 7.186(7.7); 7.181(7.6); 7.161(7.6); 7.155(7.0); 7.137(16.0); 7.130(3.9); 7.120(12.4); 6.996(0.5); 6.979(0.4); 6.909(0.9); 5.299(4.3); 4.936(1.1); 4.907(3.7); 4.877(5.0); 4.849(3.8);
4.819(1.2); 3.875(2.2); 3.844(5.5); 3.812(5.3); 3.781(2.3); 2.518(1.8); 2.490(4.5); 2.462(5.0); 2.457(5.6); 2.442(5.8); 2.408(5.2); 2.378(2.2); 2.098(2.2); 2.068(4.2); 2.063(4.8); 2.033(7.8); 2.002(4.3);
1.997(4.0); 1.966(1.9); 1.817(2.3); 1.789(2.9); 1.781(5.5); 1.754(5.3); 1.747(5.5); 1.719(5.0); 1.712(2.2); 1.684(1.4); 1.587(0.4); 1.538(99.6); 1.498(0.5); 1.293(0.6); 1.255(3.2); 0.853(0.5); 0.195(0.7);
0.044(0.5); 0.036(0.6); 0.026(0.9); 0.010(8.5); 0.000(153.5); −0.008(6.6); −0.011(6.9); −0.018(1.3); −0.020(1.2); −0.023(0.9); −0.025(0.9); −0.027(0.9); −0.198(0.7)
Example 545: ¹H-NMR(299.9 MHz, cdcl3):
δ = 7.431(0.9); 7.410(2.6); 7.388(1.3); 7.384(1.5); 7.371(0.8); 7.366(0.8); 7.346(1.4); 7.342(1.2); 7.321(0.7); 7.316(0.6); 7.277(1.0); 7.271(1.0); 7.261(3.7); 7.252(1.2); 7.247(1.1); 7.227(0.4); 7.221(0.4);
5.777(0.5); 4.990(0.5); 4.967(0.7); 4.944(0.5); 4.298(0.8); 4.273(0.8); 2.657(16.0); 2.621(0.5); 2.604(0.4); 2.598(0.6); 2.590(0.5); 2.582(0.5); 2.576(0.5); 2.570(0.4); 2.559(0.6); 2.537(0.3); 2.489(0.4);
2.480(0.3); 2.471(0.5); 2.457(0.5); 2.450(0.7); 2.438(0.6); 2.428(0.4); 2.418(0.7); 2.396(0.3); 2.349(0.4); 2.343(0.7); 2.337(0.5); 2.321(0.6); 2.315(0.7); 2.309(0.5); 2.304(0.5); 2.298(0.4); 2.293(0.3);
2.281(0.4); 2.276(0.4); 2.116(0.3); 2.096(0.6); 2.082(0.4); 2.076(0.6); 2.060(0.6); 2.057(0.6); 2.044(0.5); 2.037(0.3); 2.025(0.4); −0.001(3.0)
Example 546: ¹H-NMR(299.9 MHz, cdcl3):
δ = 7.606(1.3); 7.530(5.4); 7.504(7.0); 7.418(5.5); 7.414(5.6); 7.392(7.2); 7.388(7.6); 7.364(3.6); 7.360(3.5); 7.339(7.8); 7.335(6.5); 7.311(16.0); 7.294(15.2); 7.260(171.1); 7.247(5.4); 7.241(4.7);
7.220(6.1); 7.215(5.5); 7.195(2.4); 7.190(2.2); 6.996(14.2); 6.979(13.1); 6.908(0.9); 6.723(2.2); 5.299(1.6); 5.105(0.8); 5.086(2.6); 5.062(3.9); 5.039(2.7); 5.015(0.9); 4.348(1.5); 4.322(4.1); 4.296(4.0);
4.272(1.4); 2.803(1.3); 2.667(1.0); 2.663(1.0); 2.640(2.3); 2.634(1.5); 2.629(1.5); 2.623(2.4); 2.618(2.9); 2.603(2.7); 2.595(2.5); 2.590(1.8); 2.585(2.2); 2.580(3.4); 2.575(2.4); 2.558(2.2); 2.552(3.8);
2.529(2.1); 2.522(1.8); 2.513(2.3); 2.491(3.2); 2.481(2.7); 2.471(1.6); 2.460(3.3); 2.438(1.7); 2.392(1.4); 2.385(1.4); 2.371(2.0); 2.365(3.8); 2.359(2.8); 2.354(1.6); 2.343(3.5); 2.337(4.0); 2.331(2.6);
2.326(2.8); 2.315(1.8); 2.309(1.7); 2.303(2.2); 2.298(2.4); 2.276(1.0); 2.270(0.8); 2.175(1.8); 2.154(2.7); 2.140(2.0); 2.134(2.7); 2.122(2.9); 2.119(2.9); 2.115(2.8); 2.103(2.4); 2.084(2.1); 2.065(1.2);
1.605(0.4); 1.539(130.7); 1.293(0.5); 1.255(2.9); 0.880(0.4); 0.859(0.5); 0.830(0.4); 0.195(0.9); 0.075(0.4); 0.010(10.1); −0.001(184.4); −0.011(6.1); −0.200(0.7)
Example 547: ¹H-NMR(299.9 MHz, cdcl3):
δ = 8.100(0.4); 8.069(6.5); 8.065(6.5); 8.042(7.4); 8.038(7.4); 7.681(3.1); 7.677(3.4); 7.656(8.9); 7.652(9.0); 7.631(7.6); 7.627(7.2); 7.607(0.9); 7.591(8.0); 7.587(13.3); 7.561(16.0); 7.540(3.9); 7.535(3.8);
7.520(10.1); 7.514(8.8); 7.495(6.9); 7.490(6.5); 7.463(0.5); 7.351(6.0); 7.347(8.1); 7.339(4.3); 7.319(13.8); 7.293(5.3); 7.289(4.0); 7.261(32.3); 7.208(5.3); 7.203(5.2); 7.183(6.7); 7.178(6.6); 7.157(3.1);
7.152(2.9); 7.140(0.7); 7.135(0.7); 7.115(0.5); 7.109(0.6); 7.084(0.4); 7.079(0.4); 6.931(0.4); 6.925(0.4); 6.909(0.4); 6.152(3.3); 6.126(3.4); 4.891(1.3); 4.862(4.1); 4.833(5.8); 4.804(4.2); 4.774(1.3);
3.786(2.4); 3.754(5.8); 3.723(5.9); 3.691(2.4); 2.549(1.9); 2.520(4.7); 2.492(4.9); 2.460(2.5); 2.440(2.1); 2.408(5.2); 2.379(5.4); 2.376(5.6); 2.346(2.3); 2.185(0.3); 2.153(0.4); 2.140(0.4); 2.064(2.2);
2.033(4.3); 2.028(5.2); 1.998(7.6); 1.968(4.5); 1.963(4.5); 1.933(2.2); 1.831(2.7); 1.805(3.2); 1.797(6.1); 1.770(6.2); 1.762(5.8); 1.735(5.0); 1.727(2.2); 1.700(1.6); 1.618(0.6); 1.562(18.5); 1.293(0.5);
1.281(0.5); 1.256(2.3); 0.011(1.7); 0.000(27.5); −0.011(1.1)
Example 548: ¹H-NMR(299.9 MHz, cdcl3):
δ = 8.529(12.2); 8.521(15.5); 8.502(0.4); 8.489(0.4); 8.472(16.0); 8.465(12.6); 8.420(0.4); 8.412(0.4); 8.250(0.4); 8.242(0.3); 7.980(2.2); 7.952(2.2); 7.581(5.3); 7.576(5.4); 7.555(6.4); 7.551(6.3);
7.332(5.1); 7.328(5.8); 7.305(9.0); 7.302(9.6); 7.284(7.9); 7.279(6.2); 7.273(1.6); 7.271(1.6); 7.261(33.8); 7.245(0.8); 7.244(0.7); 7.239(0.5); 7.218(0.4); 7.212(0.4); 7.193(0.5); 7.183(4.5); 7.178(4.6);
7.157(5.9); 7.152(5.6); 7.132(2.4); 7.127(2.3); 4.930(1.1); 4.899(3.3); 4.870(4.4); 4.843(3.3); 4.812(1.1); 3.869(2.0); 3.838(4.6); 3.807(4.5); 3.775(2.0); 3.043(0.9); 2.499(1.6); 2.470(5.0); 2.438(9.9);
2.412(3.8); 2.406(5.2); 2.403(4.7); 2.373(2.0); 2.096(1.8); 2.066(3.2); 2.061(4.0); 2.030(6.4); 2.025(3.0); 2.000(3.4); 1.994(3.3); 1.964(1.6); 1.818(1.5); 1.810(0.6); 1.789(2.7); 1.781(4.8); 1.754(4.2);
1.747(4.9); 1.719(3.9); 1.711(1.9); 1.684(1.3); 1.558(27.8); 1.256(0.6); 0.010(1.3); 0.000(25.9); −0.009(0.8); −0.011(1.0)
Example 549: ¹H-NMR(299.9 MHz, cdcl3):
δ = 7.729(4.2); 7.710(4.6); 7.700(4.9); 7.681(4.5); 7.606(0.3); 7.351(5.0); 7.344(5.2); 7.327(2.7); 7.318(5.3); 7.307(6.4); 7.300(3.9); 7.285(2.7); 7.278(7.2); 7.271(4.9); 7.261(51.7); 7.251(5.1); 7.229(1.9);
7.092(2.5); 7.083(2.5); 7.063(4.2); 7.037(2.3); 7.029(2.1); 6.961(0.4); 6.937(0.7); 6.925(0.4); 6.910(0.7); 6.863(2.5); 6.855(10.0); 6.828(16.0); 6.801(8.1); 6.793(1.9); 5.570(2.3); 5.144(1.0); 5.118(3.3);
5.093(4.6); 5.068(3.3); 5.042(1.1); 4.403(1.4); 4.380(3.3); 4.355(3.4); 4.330(1.2); 2.731(1.0); 2.726(0.9); 2.705(2.4); 2.700(2.1); 2.685(3.1); 2.679(3.2); 2.665(3.6); 2.660(3.0); 2.644(2.9); 2.639(3.4);
2.618(1.4); 2.613(1.3); 2.453(0.6); 2.427(1.4); 2.412(2.5); 2.386(6.9); 2.372(5.6); 2.359(8.3); 2.342(5.7); 2.331(3.8); 2.322(3.8); 2.302(1.2); 2.280(0.7); 2.213(2.2); 2.191(2.6); 2.184(3.3); 2.173(2.3);
2.161(3.2); 2.152(3.0); 2.133(1.6); 2.122(2.4); 2.093(1.0); 1.554(60.1); 1.290(0.6); 1.267(1.2); 1.256(1.0); 1.244(0.6); 1.104(0.5); 0.000(39.1)
Example 550: ¹H-NMR(299.9 MHz, cdcl3):
δ = 7.743(1.9); 7.724(2.1); 7.714(2.3); 7.695(2.1); 7.606(0.4); 7.450(0.7); 7.443(1.3); 7.435(1.1); 7.431(1.1); 7.421(4.9); 7.415(3.6); 7.412(3.6); 7.398(2.2); 7.392(3.3); 7.380(2.5); 7.369(1.4); 7.362(3.1);
7.356(2.1); 7.351(1.4); 7.340(11.5); 7.333(16.0); 7.312(4.7); 7.310(5.4); 7.304(2.8); 7.290(0.9); 7.285(1.6); 7.281(1.3); 7.260(45.1); 7.135(1.1); 7.127(1.1); 7.103(1.7); 7.081(1.0); 7.073(0.9); 5.668(1.0);
5.650(1.1); 5.112(0.4); 5.086(1.4); 5.060(2.0); 5.036(1.4); 5.009(0.4); 4.422(0.6); 4.395(1.4); 4.373(1.4); 4.348(0.5); 2.734(0.4); 2.729(0.5); 2.715(0.6); 2.709(1.0); 2.703(0.9); 2.689(1.3); 2.683(1.4);
2.675(1.0); 2.668(1.6); 2.663(1.4); 2.657(0.8); 2.648(1.3); 2.642(1.6); 2.622(0.7); 2.617(0.6); 2.445(0.6); 2.429(1.0); 2.404(3.2); 2.387(2.3); 2.377(4.0); 2.366(1.7); 2.356(2.5); 2.349(1.6); 2.336(1.5);
2.315(0.6); 2.209(0.9); 2.185(1.0); 2.180(1.5); 2.169(0.9); 2.158(1.4); 2.148(1.4); 2.139(1.2); 2.129(0.7); 2.117(1.1); 2.113(0.8); 2.088(0.4); 1.554(39.4); 1.283(0.3); 1.254(1.0); 0.010(1.7); −0.001(37.7); −0.011
(1.5)
Example 551: ¹H-NMR(399.8 MHz, cdcl3):
δ = 7.692(0.9); 7.674(0.9); 7.528(13.2); 7.509(15.2); 7.478(5.0); 7.470(6.0); 7.464(5.8); 7.456(6.6); 7.429(8.1); 7.411(6.3); 7.363(12.2); 7.343(16.0); 7.321(15.6); 7.313(6.5); 7.302(7.5); 7.297(7.5);
7.288(10.9); 7.260(72.5); 6.995(0.4); 6.466(2.7); 6.450(2.7); 5.216(0.9); 5.197(2.9); 5.181(3.7); 5.160(3.2); 5.138(1.0); 4.482(1.7); 4.461(4.1); 4.441(3.9); 4.420(1.4); 2.780(0.7); 2.757(1.9); 2.749(2.0);
2.742(2.4); 2.734(3.1); 2.727(2.6); 2.720(2.3); 2.712(2.9); 2.704(2.5); 2.696(2.6); 2.690(2.6); 2.681(2.1); 2.670(2.3); 2.652(3.2); 2.638(2.4); 2.624(2.2); 2.608(1.2); 2.587(0.3); 2.489(0.9); 2.482(1.1);
2.466(2.4); 2.459(2.3); 2.450(2.9); 2.442(3.4); 2.419(2.6); 2.390(2.3); 2.368(2.2); 2.359(2.5); 2.344(3.2); 2.328(2.1); 2.317(1.5); 1.545(40.5); 1.279(0.7); 1.262(0.8); 0.146(0.5); 0.050(0.5); 0.000(82.6); −0.149
(0.4)
Example 552: ¹H-NMR(399.8 MHz, cdcl3):
δ = 7.727(0.4); 7.677(0.5); 7.540(0.5); 7.519(1.4); 7.490(4.9); 7.472(13.6); 7.468(12.2); 7.433(0.9); 7.411(5.0); 7.387(9.1); 7.365(8.3); 7.335(10.2); 7.324(10.3); 7.301(16.0); 7.281(12.5); 7.260(153.5);
6.996(0.8); 6.413(2.6); 6.395(2.6); 5.300(0.5); 5.186(0.9); 5.164(2.5); 5.147(3.5); 5.126(2.8); 5.105(0.9); 4.481(1.6); 4.460(3.7); 4.439(3.6); 4.417(1.2); 3.494(0.4); 3.466(0.4); 2.783(0.6); 2.760(1.6);
2.752(1.8); 2.745(2.2); 2.738(2.7); 2.715(2.8); 2.707(2.7); 2.685(2.6); 2.661(2.9); 2.633(2.0); 2.619(1.0); 2.603(0.3); 2.473(2.1); 2.448(3.1); 2.427(2.3); 2.390(1.3); 2.357(2.3); 2.343(2.9); 2.328(2.0);
2.287(0.4); 1.629(0.3); 1.538(103.0); 1.497(0.4); 1.279(0.8); 1.263(1.1); 0.834(0.4); 0.824(0.4); 0.146(0.8); 0.000(171.5); −0.048(0.4); −0.150(0.8)
Example 553: ¹H-NMR(399.8 MHz, cdcl3):
δ = 7.703(0.4); 7.677(0.3); 7.601(0.4); 7.588(0.8); 7.518(2.2); 7.502(10.7); 7.483(13.2); 7.406(1.7); 7.388(11.4); 7.368(16.0); 7.344(8.1); 7.330(8.5); 7.310(6.3); 7.301(9.8); 7.282(28.4); 7.273(44.0);
7.259(239.2); 7.233(9.2); 7.223(8.1); 7.214(8.6); 7.204(8.0); 7.193(3.7); 7.182(0.6); 6.995(1.4); 6.495(3.8); 6.473(3.9); 6.262(0.3); 5.266(1.7); 5.244(4.9); 5.225(5.4); 5.204(4.1); 5.181(1.4); 4.490(2.6);
4.468(5.6); 4.448(5.5); 4.428(2.5); 3.729(0.4); 3.707(0.3); 3.556(0.4); 3.540(0.3); 3.518(0.4); 3.440(0.3); 3.222(0.4); 3.102(0.4); 3.087(0.4); 2.772(0.3); 2.742(1.0); 2.724(2.6); 2.712(3.5); 2.703(4.3);
2.694(4.1); 2.687(4.0); 2.661(2.3); 2.651(1.9); 2.614(1.7); 2.590(3.6); 2.573(5.3); 2.561(4.5); 2.543(3.1); 2.521(1.4); 2.483(1.9); 2.466(5.4); 2.460(4.5); 2.449(7.1); 2.443(7.8); 2.427(10.7); 2.417(6.8);
2.409(7.0); 2.390(2.8); 2.371(0.9); 2.133(1.3); 2.109(1.1); 2.072(0.3); 2.055(0.3); 2.007(0.5); 1.984(0.4); 1.742(0.4); 1.539(84.3); 1.432(0.6); 1.413(1.1); 1.393(0.6); 1.364(0.4); 1.339(0.4); 1.288(0.7);
1.254(2.4); 1.232(0.4); 0.880(0.4); 0.851(0.4); 0.146(1.4); 0.099(0.4); 0.007(16.8); −0.001(277.2); −0.150(1.2)
Example 554: ¹H-NMR(299.9 MHz, cdcl3):
δ = 7.503(1.1); 7.498(1.1); 7.477(1.2); 7.473(1.2); 7.348(0.9); 7.343(1.0); 7.321(1.2); 7.317(1.4); 7.307(0.7); 7.303(0.6); 7.282(1.3); 7.278(1.2); 7.261(4.1); 7.253(0.8); 7.198(0.9); 7.193(0.9); 7.172(1.1);
7.167(1.0); 7.147(0.4); 7.142(0.4); 6.447(0.5); 6.425(0.5); 4.768(0.6); 4.740(0.9); 4.711(0.6); 3.809(0.4); 3.778(0.9); 3.747(1.0); 3.716(0.3); 2.718(16.0); 2.462(0.8); 2.434(0.9); 2.429(1.4); 2.404(1.2);
2.369(0.8); 2.366(0.9); 2.336(0.4); 2.034(0.3); 2.003(0.7); 1.999(0.7); 1.968(1.2); 1.938(0.7); 1.932(0.7); 1.843(0.3); 1.815(0.6); 1.807(1.0); 1.781(0.9); 1.773(1.0); 1.746(0.8); 1.738(0.4); 1.710(0.3);
0.000(3.2)
Example 555: ¹H-NMR(299.9 MHz, cdcl3):
δ = 8.639(8.7); 8.626(8.6); 8.540(0.3); 8.432(13.0); 7.848(6.3); 7.841(9.7); 7.835(6.7); 7.822(7.0); 7.815(10.6); 7.809(7.0); 7.606(1.1); 7.510(9.5); 7.485(14.2); 7.455(8.9); 7.451(10.0); 7.429(12.1);
7.425(15.0); 7.419(8.5); 7.414(7.6); 7.394(13.2); 7.389(11.5); 7.368(6.9); 7.364(5.7); 7.315(10.3); 7.309(16.0); 7.290(19.7); 7.283(18.1); 7.260(178.2); 7.192(0.5); 7.172(0.6); 7.145(0.6); 7.119(0.4);
6.909(1.0); 5.832(0.4); 5.806(0.4); 5.782(0.4); 5.652(4.3); 5.299(1.5); 5.164(2.0); 5.137(5.8); 5.112(8.3); 5.087(5.7); 5.062(1.8); 4.380(2.6); 4.355(6.2); 4.331(5.9); 4.305(2.3); 3.631(0.4); 3.600(0.4);
2.802(0.4); 2.677(1.6); 2.671(1.7); 2.660(2.3); 2.653(3.6); 2.632(5.1); 2.626(5.0); 2.615(4.7); 2.608(5.1); 2.593(4.1); 2.587(4.5); 2.568(2.4); 2.562(2.3); 2.483(1.5); 2.463(2.5); 2.442(4.8); 2.423(6.8);
2.406(8.4); 2.391(8.1); 2.376(11.5); 2.349(9.1); 2.336(4.1); 2.324(3.5); 2.308(3.5); 2.280(1.3); 2.149(3.2); 2.125(5.4); 2.112(4.1); 2.090(5.8); 2.054(3.8); 2.029(2.0); 1.952(6.5); 1.868(0.5); 1.838(0.6);
1.811(0.5); 1.776(0.5); 1.665(0.6); 1.631(1.2); 1.553(134.7); 1.415(0.5); 1.333(0.6); 1.294(1.1); 1.254(8.2); 0.902(0.4); 0.879(0.6); 0.855(0.7); 0.195(0.9); 0.169(1.2); 0.069(1.3); −0.001(175.3); −0.011(9.1);
−0.199(0.7)
Example 556: ¹H-NMR(299.9 MHz, dmso):
δ = 8.225(0.5); 8.080(4.4); 8.052(1.9); 8.024(1.8); 7.726(0.4); 7.691(1.6); 7.671(1.7); 7.662(1.8); 7.642(1.6); 7.553(1.6); 7.524(1.6); 7.517(1.6); 7.247(0.5); 7.222(1.0); 7.193(1.6); 7.165(0.9); 7.112(1.5);
6.931(3.2); 6.750(1.6); 4.795(0.8); 4.771(1.2); 4.748(1.0); 4.721(0.4); 4.122(1.2); 4.098(1.1); 3.906(2.1); 3.875(16.0); 3.316(31.7); 2.507(23.8); 2.502(29.7); 2.496(23.3); 2.471(2.1); 2.456(1.9); 2.447(1.9);
2.423(1.3); 2.393(0.9); 2.372(0.4); 2.256(0.8); 2.229(1.1); 2.199(0.9); 2.160(0.9); 2.126(1.0); 2.111(1.1); 2.094(1.0); 0.000(12.0)
Example 557: ¹H-NMR(299.9 MHz, cdcl3):
δ = 7.780(0.4); 7.718(6.0); 7.699(6.6); 7.689(6.9); 7.670(6.2); 7.621(0.9); 7.606(1.2); 7.593(1.0); 7.547(13.5); 7.519(16.0); 7.462(0.5); 7.390(6.9); 7.382(7.0); 7.356(7.0); 7.349(7.0); 7.277(16.6);
7.271(23.4); 7.260(117.5); 7.251(17.1); 7.244(10.2); 7.223(9.9); 7.216(7.7); 7.185(0.6); 7.102(3.5); 7.094(3.5); 7.074(6.0); 7.048(3.3); 7.041(2.9); 6.909(0.6); 6.066(3.7); 6.046(3.6); 5.099(1.6); 5.072(4.9);
5.047(6.4); 5.023(4.8); 4.998(1.6); 4.418(2.0); 4.393(4.9); 4.368(4.7); 4.343(1.7); 2.733(1.4); 2.712(3.7); 2.691(4.2); 2.686(4.5); 2.670(4.8); 2.649(4.5); 2.625(2.1); 2.496(0.4); 2.468(0.8); 2.444(1.7);
2.428(4.2); 2.419(4.9); 2.402(10.8); 2.389(6.2); 2.374(12.0); 2.347(7.1); 2.326(1.2); 2.221(3.0); 2.198(3.5); 2.192(4.2); 2.181(3.1); 2.170(4.3); 2.161(3.9); 2.152(3.6); 2.140(2.1); 2.131(3.3); 2.102(1.3);
1.982(0.3); 1.841(0.4); 1.541(40.8); 1.255(1.9); 1.064(0.5); 0.882(0.9); 0.858(0.8); 0.830(0.5); 0.196(0.6); 0.069(2.1); 0.000(125.6); −0.199(0.5)
Example 558: ¹H-NMR(400.0 MHz, CD3CN):
δ = 7.914(8.7); 7.911(8.7); 7.894(9.6); 7.891(9.5); 7.630(3.7); 7.627(4.1); 7.611(9.7); 7.608(10.0); 7.593(8.1); 7.589(7.9); 7.579(7.3); 7.575(8.0); 7.559(8.2); 7.556(8.8); 7.540(3.3); 7.537(3.1); 7.515(0.6);
7.490(15.0); 7.485(16.0); 7.297(7.0); 7.292(7.1); 7.276(9.5); 7.271(9.6); 7.174(15.5); 7.153(11.0); 7.098(9.2); 7.094(10.0); 7.079(8.0); 7.076(8.7); 6.678(4.1); 3.369(2.2); 3.358(4.1); 3.350(5.1); 3.339(8.0);
3.329(5.0); 3.321(4.3); 3.310(2.1); 2.521(3.4); 2.502(7.4); 2.480(7.7); 2.462(4.0); 2.165(549.2); 2.120(1.2); 2.114(1.4); 2.108(1.7); 2.102(1.2); 2.097(0.7); 1.965(18.7); 1.959(35.0); 1.953(113.6);
1.947(194.0); 1.941(248.5); 1.934(175.9); 1.928(91.1); 1.781(0.7); 1.775(1.1); 1.769(1.5); 1.763(1.1); 1.757(0.6); 1.475(3.9); 1.458(6.6); 1.455(6.6); 1.439(6.5); 1.436(7.3); 1.417(4.4); 1.329(4.8);
1.317(5.8); 1.312(8.1); 1.301(7.5); 1.296(5.5); 1.284(3.7); 1.270(0.9); 0.147(0.7); 0.000(144.2); −0.149(0.7)
Example 559: ¹H-NMR(299.9 MHz, cdcl3):
δ = 8.531(0.4); 8.523(0.5); 8.515(0.3); 8.506(0.4); 8.475(0.6); 8.466(0.7); 8.447(14.0); 8.439(15.0); 8.419(0.5); 8.291(16.0); 8.284(14.5); 7.718(0.4); 7.681(4.4); 7.662(4.8); 7.652(5.1); 7.633(4.8);
7.607(0.6); 7.405(5.3); 7.398(5.4); 7.372(5.4); 7.365(5.5); 7.308(2.9); 7.280(3.5); 7.261(73.9); 7.079(2.8); 7.072(2.7); 7.050(4.6); 7.025(2.5); 7.018(2.3); 6.910(0.5); 5.057(1.2); 5.031(3.6); 5.010(4.5);
4.983(3.8); 4.957(1.3); 4.425(1.4); 4.399(4.0); 4.375(3.8); 4.348(1.5); 3.164(0.4); 3.153(0.4); 3.135(0.7); 3.115(0.5); 2.836(0.7); 2.812(0.5); 2.804(0.6); 2.739(0.9); 2.734(1.0); 2.714(3.0); 2.709(2.9);
2.698(1.7); 2.686(3.5); 2.672(3.8); 2.667(3.2); 2.660(2.1); 2.645(3.6); 2.624(1.8); 2.620(1.7); 2.449(6.9); 2.421(13.5); 2.396(16.0); 2.372(6.4); 2.234(2.1); 2.211(3.1); 2.206(2.5); 2.201(2.4); 2.191(3.3);
2.178(3.0); 2.174(3.3); 2.164(3.3); 2.144(2.6); 2.117(1.1); 1.979(0.4); 1.950(0.3); 1.862(0.4); 1.829(0.5); 1.800(0.5); 1.762(0.6); 1.733(0.8); 1.581(6.6); 1.397(0.8); 1.362(0.5); 1.329(0.7); 1.318(0.8);
1.305(1.1); 1.282(1.0); 1.255(1.3); 1.216(0.4); 1.166(0.5); 1.142(0.7); 1.118(0.4); 0.904(0.8); 0.882(1.9); 0.858(1.1); 0.840(0.6); 0.069(0.8); 0.010(3.1); −0.001(59.5); −0.011(3.5)
Example 560: ¹H-NMR(299.9 MHz, cdcl3):
δ = 7.709(5.5); 7.688(7.1); 7.663(5.5); 7.602(1.2); 7.575(1.2); 7.542(1.2); 7.475(8.0); 7.449(9.2); 7.373(10.5); 7.337(16.0); 7.258(38.4); 7.216(8.2); 7.191(8.4); 7.169(3.8); 7.079(4.7); 7.053(7.0); 7.029(3.9);
5.804(4.8); 5.076(4.9); 5.053(6.6); 5.029(4.8); 4.391(6.0); 4.368(5.8); 4.151(0.4); 2.709(4.1); 2.684(5.3); 2.671(5.5); 2.646(4.8); 2.621(2.3); 2.527(0.6); 2.421(7.1); 2.396(14.6); 2.369(15.0); 2.345(7.1);
2.231(3.2); 2.204(5.3); 2.173(5.3); 2.145(3.6); 2.117(1.4); 2.040(0.5); 1.985(0.3); 1.788(0.3); 1.710(0.3); 1.657(0.5); 1.546(30.0); 1.252(0.9); 0.848(0.3); −0.003(34.0)
Example 561: ¹H-NMR(299.9 MHz, cdcl3):
δ = 8.195(0.7); 8.166(0.7); 7.974(5.6); 7.970(4.2); 7.966(3.6); 7.950(5.9); 7.943(6.5); 7.671(0.4); 7.649(0.5); 7.625(0.8); 7.604(1.9); 7.584(1.0); 7.558(9.2); 7.533(16.0); 7.525(10.0); 7.517(14.6); 7.509(7.7);
7.499(7.3); 7.493(6.7); 7.474(2.5); 7.468(1.9); 7.446(1.2); 7.432(1.6); 7.408(2.8); 7.382(4.6); 7.363(4.7); 7.336(2.7); 7.314(5.1); 7.258(238.4); 7.191(0.8); 7.140(0.4); 7.024(7.2); 7.017(6.8); 7.004(4.3);
7.000(4.8); 6.994(6.5); 6.970(0.8); 6.945(0.5); 6.907(1.5); 6.288(0.3); 6.083(2.7); 6.053(2.8); 5.298(1.0); 5.244(1.0); 5.216(2.9); 5.188(3.3); 5.167(2.8); 5.137(0.9); 4.506(1.4); 4.483(3.6); 4.455(3.9);
4.424(1.6); 4.141(0.7); 2.807(0.6); 2.775(1.5); 2.764(1.9); 2.755(2.4); 2.744(3.0); 2.733(2.5); 2.717(3.0); 2.704(2.1); 2.686(1.5); 2.674(1.2); 2.646(0.9); 2.611(2.3); 2.589(3.2); 2.570(3.0); 2.553(2.6);
2.519(2.1); 2.481(2.9); 2.471(2.5); 2.459(2.9); 2.450(3.5); 2.439(2.5); 2.420(4.5); 2.406(3.8); 2.402(3.8); 2.394(3.7); 2.381(2.9); 2.374(2.9); 2.356(1.7); 2.334(1.3); 2.322(0.6); 2.238(0.4); 2.043(0.5);
1.602(0.6); 1.535(171.6); 1.309(0.6); 1.286(0.5); 1.254(0.8); 1.066(0.5); 0.848(0.4); 0.194(1.3); 0.083(0.4); 0.064(0.9); 0.008(17.0); −0.002(259.6); −0.012(12.6); −0.069(0.4); −0.201(1.2)
Example 562: ¹H-NMR(299.9 MHz, cdcl3):
δ = 16.012(0.4); 14.228(0.3); 8.906(0.3); 8.827(0.4); 8.810(0.5); 8.674(14.7); 8.623(0.7); 8.564(0.4); 8.372(0.4); 8.171(0.4); 7.936(6.5); 7.930(4.9); 7.916(4.9); 7.909(6.8); 7.607(0.9); 7.547(0.4); 7.496(5.1);
7.483(6.9); 7.474(5.7); 7.465(7.2); 7.423(1.0); 7.368(2.3); 7.349(15.1); 7.341(12.6); 7.322(16.0); 7.305(14.3); 7.260(165.9); 6.909(0.9); 6.549(0.4); 6.484(3.3); 6.462(3.2); 5.233(0.9); 5.205(3.1); 5.182(4.2);
5.155(3.5); 5.124(1.2); 4.508(1.8); 4.478(4.5); 4.451(4.6); 4.425(1.7); 3.661(0.3); 3.532(0.5); 2.777(2.4); 2.755(3.6); 2.746(3.0); 2.725(4.6); 2.694(4.1); 2.669(3.8); 2.635(2.8); 2.491(2.9); 2.471(3.5);
2.461(3.9); 2.429(3.7); 2.414(3.4); 2.406(3.4); 2.397(3.3); 2.389(3.5); 2.363(3.6); 2.341(2.4); 1.762(0.4); 1.685(0.5); 1.634(0.8); 1.549(52.9); 1.476(0.6); 1.416(0.7); 1.369(0.4); 1.332(0.6); 1.285(1.3);
1.255(3.0); 0.853(0.4); 0.195(1.0); 0.069(1.0); 0.000(177.3); −0.200(0.8); −1.384(0.4)
Example 563: ¹H-NMR(299.9 MHz, cdcl3):
δ = 7.950(0.8); 7.926(0.8); 7.712(1.0); 7.686(1.1); 7.569(0.6); 7.510(6.6); 7.484(13.6); 7.458(7.2); 7.420(1.6); 7.379(4.0); 7.354(5.9); 7.333(6.2); 7.310(8.3); 7.261(26.2); 7.249(10.9); 7.220(10.4);
7.210(8.4); 7.186(16.0); 7.163(6.4); 6.256(3.2); 6.235(3.2); 5.272(1.2); 5.243(3.3); 5.219(4.0); 5.192(3.0); 5.161(1.0); 4.500(2.0); 4.473(4.1); 4.450(4.0); 4.420(1.8); 2.699(3.6); 2.687(3.6); 2.657(2.2);
2.644(2.0); 2.594(3.1); 2.573(4.1); 2.557(3.8); 2.533(3.0); 2.501(2.8); 2.473(4.7); 2.445(6.4); 2.423(7.2); 2.401(4.7); 2.377(2.1); 2.235(0.4); 2.215(0.4); 2.187(0.4); 2.028(0.3); 1.988(0.4); 1.478(0.5);
1.258(0.5); 0.001(23.2)
Example 564: ¹H-NMR(299.9 MHz, cdcl3):
δ = 7.607(0.5); 7.504(5.4); 7.477(7.1); 7.453(0.5); 7.405(0.6); 7.391(0.9); 7.376(1.7); 7.349(4.6); 7.322(7.7); 7.315(6.2); 7.305(6.6); 7.278(11.8); 7.260(72.7); 7.259(72.7); 7.230(2.0); 6.935(0.4); 6.908(1.1);
6.856(9.7); 6.829(16.0); 6.802(8.2); 6.214(2.4); 5.264(1.0); 5.236(2.9); 5.211(3.5); 5.185(2.7); 5.154(0.8); 4.490(1.5); 4.463(3.6); 4.436(4.0); 4.408(1.6); 2.738(1.7); 2.708(2.8); 2.696(2.5); 2.680(3.0);
2.668(2.2); 2.650(1.6); 2.637(1.7); 2.594(2.5); 2.573(3.2); 2.552(3.1); 2.536(2.1); 2.502(1.5); 2.468(2.6); 2.438(3.7); 2.420(4.2); 2.411(5.2); 2.397(4.5); 2.389(4.0); 2.375(3.1); 2.367(3.0); 2.352(1.7);
2.317(0.5); 1.553(18.8); 0.195(0.4); 0.000(74.7)
Example 565: ¹H-NMR(400.0 MHz, CD3CN):
δ = 8.860(16.0); 8.748(9.8); 8.736(9.7); 7.588(0.5); 7.499(14.1); 7.494(15.3); 7.310(6.1); 7.305(6.1); 7.289(8.1); 7.284(8.2); 7.163(13.0); 7.142(9.9); 6.996(9.6); 6.983(9.2); 6.670(2.7); 5.448(0.7);
3.396(1.7); 3.385(3.6); 3.377(4.1); 3.366(7.4); 3.354(4.2); 3.347(3.9); 3.335(1.9); 2.527(2.8); 2.508(6.0); 2.486(6.4); 2.468(4.2); 2.463(2.5); 2.458(1.5); 2.453(0.8); 2.404(0.4); 2.156(592.7); 2.120(2.3);
2.114(3.2); 2.107(3.9); 2.101(2.8); 2.095(1.6); 2.076(0.6); 1.964(24.8); 1.958(51.1); 1.952(242.5); 1.946(434.6); 1.940(578.2); 1.934(402.0); 1.928(207.7); 1.781(1.4); 1.775(2.6); 1.769(3.4); 1.762(2.4);
1.756(1.3); 1.471(3.7); 1.455(5.3); 1.452(5.6); 1.449(4.9); 1.435(5.2); 1.432(6.1); 1.430(5.2); 1.413(4.2); 1.310(4.2); 1.299(4.7); 1.293(6.8); 1.281(6.7); 1.276(5.0); 1.265(3.9); 0.146(1.5); 0.008(14.3);
0.000(360.4); −0.150(1.6)
Example 566: ¹H-NMR(400.0 MHz, CD3CN):
δ = 8.737(10.3); 8.731(12.7); 8.718(0.6); 8.690(12.6); 8.685(9.8); 7.472(15.1); 7.467(15.5); 7.241(5.8); 7.235(5.5); 7.220(9.9); 7.214(9.5); 7.160(16.0); 7.139(9.4); 7.125(0.3); 7.057(2.4); 5.447(0.4);
4.068(0.6); 4.050(0.6); 3.431(2.0); 3.419(4.1); 3.412(4.6); 3.408(2.7); 3.400(8.4); 3.393(3.1); 3.389(4.6); 3.381(4.4); 3.370(2.1); 2.541(3.0); 2.523(6.5); 2.501(6.7); 2.482(3.1); 2.135(86.2); 2.120(0.9);
2.113(1.1); 2.107(1.4); 2.101(0.9); 2.095(0.5); 1.972(3.1); 1.964(8.6); 1.958(17.9); 1.952(87.0); 1.946(155.6); 1.940(205.3); 1.934(139.5); 1.927(70.7); 1.781(0.5); 1.774(0.9); 1.768(1.2); 1.762(0.8);
1.756(0.4); 1.515(4.2); 1.499(6.3); 1.496(5.8); 1.493(4.8); 1.480(6.0); 1.476(6.5); 1.474(5.1); 1.457(4.7); 1.342(4.4); 1.331(5.1); 1.325(6.6); 1.314(6.6); 1.308(4.2); 1.297(3.6); 1.221(0.7); 1.204(1.4);
1.186(0.7); 0.146(0.6); 0.008(5.6); 0.000(147.7); −0.009(4.8); −0.150(0.7)
Example 567: ¹H-NMR(400.0 MHz, CD3CN):
δ = 8.437(7.8); 8.431(15.7); 8.420(16.0); 8.414(8.0); 7.476(10.6); 7.471(11.3); 7.240(4.1); 7.235(4.0); 7.219(6.9); 7.214(7.0); 7.159(11.7); 7.138(7.3); 7.109(1.7); 5.447(3.0); 4.068(0.4); 4.050(0.4);
3.423(1.4); 3.411(3.1); 3.404(3.3); 3.400(2.0); 3.392(6.2); 3.385(2.1); 3.381(3.3); 3.373(3.2); 3.362(1.6); 2.546(2.2); 2.528(4.7); 2.505(4.9); 2.487(2.3); 2.146(216.3); 2.120(1.4); 2.114(1.7); 2.107(2.0);
2.101(1.4); 2.095(0.8); 1.972(2.6); 1.964(11.8); 1.958(24.3); 1.952(119.8); 1.946(216.1); 1.940(285.7); 1.934(195.2); 1.928(99.4); 1.781(0.7); 1.775(1.2); 1.768(1.7); 1.762(1.2); 1.756(0.6); 1.506(3.0);
1.490(4.5); 1.487(4.2); 1.484(3.6); 1.471(4.4); 1.468(4.7); 1.465(3.8); 1.449(3.4); 1.347(3.2); 1.336(3.7); 1.330(5.0); 1.319(4.9); 1.313(3.1); 1.302(2.6); 1.270(0.3); 1.222(0.5); 1.204(1.0); 1.186(0.5);
0.146(0.7); 0.008(6.3); 0.000(175.4); −0.009(6.2); −0.150(0.7)
Example 568: ¹H-NMR(400.0 MHz, CD3CN):
δ = 7.486(2.8); 7.481(3.0); 7.288(1.3); 7.283(1.2); 7.267(1.6); 7.262(1.6); 7.115(2.5); 7.094(2.0); 6.763(0.6); 3.778(0.6); 3.347(0.7); 3.339(0.8); 3.328(1.3); 3.317(0.8); 3.309(0.7); 3.298(0.4); 2.623(16.0);
2.522(0.6); 2.503(1.3); 2.482(1.3); 2.463(0.7); 2.174(34.1); 2.168(34.5); 1.972(0.7); 1.965(1.2); 1.953(12.3); 1.947(22.1); 1.941(29.8); 1.935(21.0); 1.929(11.1); 1.451(0.6); 1.433(1.2); 1.413(1.2);
1.393(0.7); 1.313(0.7); 1.296(1.3); 1.284(1.3); 1.267(0.6); 1.204(0.3); 0.000(14.4)
Example 569: ¹H-NMR(399.8 MHz, cdcl3):
δ = 7.756(1.0); 7.742(1.1); 7.735(1.1); 7.720(1.0); 7.381(1.2); 7.376(1.2); 7.356(1.2); 7.351(1.2); 7.286(29.8); 7.151(0.6); 7.145(0.6); 7.130(1.0); 7.110(0.5); 5.821(0.6); 4.979(0.8); 4.960(1.0); 4.943(0.8);
4.393(0.8); 4.374(0.8); 2.739(0.5); 2.708(0.8); 2.686(16.0); 2.660(0.8); 2.642(0.3); 2.440(1.2); 2.421(2.6); 2.400(2.8); 2.381(1.2); 2.213(0.4); 2.191(0.6); 2.175(0.6); 2.167(0.6); 2.161(0.6); 2.145(0.5);
1.567(14.1); 1.281(1.2); 0.112(1.1); 0.095(30.2); 0.070(0.6); 0.025(34.7)
Example 570: ¹H-NMR(299.9 MHz, cdcl3):
δ = 16.752(0.3); 8.002(0.8); 7.726(4.6); 7.707(5.0); 7.697(5.3); 7.677(4.8); 7.606(2.7); 7.484(5.0); 7.476(5.0); 7.451(4.9); 7.444(5.0); 7.395(0.6); 7.377(0.7); 7.326(14.3); 7.309(15.6); 7.259(433.8);
7.194(1.1); 7.167(0.7); 7.131(0.8); 7.114(0.9); 7.087(2.7); 7.080(2.9); 7.058(4.8); 7.034(3.0); 7.014(15.4); 6.997(13.9); 6.908(2.6); 6.743(0.4); 6.676(2.6); 5.108(1.3); 5.082(3.6); 5.058(5.0); 5.033(3.9);
5.008(1.3); 4.396(3.6); 4.369(3.6); 4.346(1.3); 2.731(1.0); 2.708(2.8); 2.688(3.3); 2.671(3.7); 2.666(3.3); 2.646(3.5); 2.624(1.5); 2.619(1.6); 2.445(6.4); 2.418(11.0); 2.393(16.0); 2.369(7.3); 2.268(2.3);
2.246(3.1); 2.236(2.3); 2.226(2.3); 2.210(3.1); 2.199(2.8); 2.178(2.6); 2.151(0.9); 2.073(0.5); 2.054(0.5); 2.006(0.4); 1.795(0.4); 1.683(0.4); 1.539(315.0); 1.402(0.6); 1.255(4.1); 0.880(0.8); 0.852(0.8);
0.195(2.0); 0.131(0.4); 0.068(1.5); 0.010(23.6); −0.001(407.3); −0.012(18.9); −0.068(0.6); −0.200(2.0)
Example 571: ¹H-NMR(299.9 MHz, cdcl3):
δ = 8.393(9.2); 8.387(9.9); 8.378(9.8); 8.371(9.7); 8.067(10.6); 8.060(10.5); 8.041(11.4); 8.034(10.9); 7.731(6.6); 7.712(7.1); 7.702(7.4); 7.683(7.1); 7.607(1.1); 7.413(6.9); 7.406(7.2); 7.380(6.9);
7.373(7.3); 7.331(0.5); 7.300(12.0); 7.284(12.8); 7.274(13.8); 7.261(184.4); 7.197(0.6); 7.162(0.4); 7.117(3.8); 7.109(3.8); 7.087(6.2); 7.063(3.4); 7.055(3.2); 6.909(1.0); 6.376(3.5); 6.362(3.5); 5.111(1.7);
5.086(5.3); 5.061(7.2); 5.037(5.5); 5.012(2.0); 4.437(1.9); 4.414(5.0); 4.388(5.2); 4.361(2.0); 4.221(0.6); 2.748(1.1); 2.725(3.8); 2.705(4.0); 2.700(4.5); 2.683(4.8); 2.662(4.8); 2.639(2.2); 2.634(2.1);
2.463(0.8); 2.443(4.9); 2.420(14.4); 2.393(16.0); 2.368(9.8); 2.350(1.0); 2.244(3.0); 2.214(4.4); 2.204(2.9); 2.193(4.3); 2.185(4.1); 2.176(4.2); 2.166(2.2); 2.155(3.7); 2.124(1.5); 1.547(116.6); 1.385(0.4);
1.363(0.4); 1.316(0.6); 1.255(3.8); 1.228(0.6); 1.141(0.4); 0.880(0.7); 0.850(0.6); 0.195(0.6); 0.069(0.6); 0.011(6.8); 0.000(161.7); −0.011(8.7); −0.077(0.5); −0.135(0.3); −0.199(0.8); −0.357(0.3)
Example 572: ¹H-NMR(299.9 MHz, cdcl3):
δ = 8.745(0.4); 8.695(6.7); 8.682(6.9); 8.004(0.4); 7.849(0.5); 7.728(5.2); 7.709(5.5); 7.699(5.9); 7.680(5.5); 7.646(6.3); 7.621(8.4); 7.555(1.1); 7.545(0.7); 7.533(0.6); 7.519(0.5); 7.486(7.6); 7.470(7.5);
7.460(5.8); 7.444(5.5); 7.329(5.9); 7.321(6.1); 7.296(6.2); 7.288(6.9); 7.261(120.3); 7.228(0.6); 7.184(0.4); 7.148(0.3); 7.095(3.0); 7.087(2.9); 7.063(5.0); 7.041(2.7); 7.033(2.6); 6.910(0.7); 5.493(0.3);
5.429(3.3); 5.409(3.5); 5.064(1.4); 5.038(4.0); 5.014(5.1); 4.991(4.0); 4.965(1.3); 4.421(1.6); 4.395(4.3); 4.368(4.3); 4.341(1.5); 4.221(0.4); 2.743(1.1); 2.719(3.0); 2.697(3.8); 2.681(3.8); 2.656(3.9);
2.632(1.6); 2.482(0.6); 2.432(6.4); 2.406(14.0); 2.379(16.0); 2.354(6.9); 2.311(0.4); 2.214(2.6); 2.184(3.2); 2.171(3.0); 2.164(3.3); 2.155(3.2); 2.146(3.3); 2.124(2.7); 2.098(1.1); 2.005(0.3); 1.971(0.5);
1.856(0.4); 1.821(0.4); 1.792(0.4); 1.703(0.4); 1.548(90.9); 1.259(0.6); 0.857(0.3); 0.196(0.5); 0.000(110.7); −0.198(0.6)
Example 573: ¹H-NMR(399.8 MHz, cdcl3):
δ = 7.519(0.4); 7.507(1.1); 7.487(1.4); 7.387(0.4); 7.366(0.8); 7.353(0.9); 7.332(1.3); 7.310(0.6); 7.301(1.0); 7.278(0.9); 7.261(45.6); 6.410(0.4); 6.397(0.4); 5.300(0.6); 5.143(0.5); 5.124(0.7); 5.105(0.5);
4.437(0.7); 4.417(0.7); 2.700(0.9); 2.693(0.6); 2.666(16.0); 2.587(0.4); 2.569(0.6); 2.557(0.6); 2.544(0.3); 2.458(0.4); 2.435(0.6); 2.426(0.7); 2.418(0.8); 2.410(1.1); 2.404(0.8); 2.394(0.6); 2.376(0.3);
1.538(29.6); 0.000(53.3); −0.008(2.3)
Example 574: ¹H-NMR(399.8 MHz, cdcl3):
δ = 7.903(0.3); 7.809(1.1); 7.771(4.9); 7.519(0.5); 7.452(1.6); 7.430(2.1); 7.411(0.7); 7.393(0.6); 7.322(0.7); 7.300(2.3); 7.284(5.4); 7.260(65.0); 6.996(0.4); 6.940(0.5); 6.804(0.9); 6.756(1.6); 6.669(0.6);
6.620(3.8); 6.485(1.6); 5.201(0.4); 5.180(1.2); 5.161(1.2); 5.142(0.9); 4.430(0.6); 4.408(1.1); 4.391(1.0); 3.917(1.0); 3.885(3.8); 3.842(16.0); 3.809(0.7); 3.680(0.4); 3.660(0.4); 2.655(1.0); 2.634(0.7);
2.624(0.5); 2.544(1.3); 2.529(1.0); 2.507(0.8); 2.461(0.6); 2.455(0.7); 2.438(1.3); 2.422(2.1); 2.416(2.0); 2.401(2.1); 2.379(0.9); 2.349(0.3); 1.543(33.9); 1.255(0.9); 0.000(73.5); −0.050(0.4); −0.150(0.4)
Example 575: ¹H-NMR(399.8 MHz, cdcl3):
δ = 8.521(0.4); 8.443(1.1); 8.427(1.0); 8.392(12.0); 8.387(13.8); 8.380(13.3); 8.375(13.2); 8.315(0.5); 8.072(0.3); 8.048(0.5); 8.025(0.6); 7.910(12.5); 7.905(13.5); 7.891(13.4); 7.886(13.9); 7.679(0.6);
7.645(0.4); 7.564(0.6); 7.545(0.8); 7.520(2.5); 7.506(13.1); 7.486(16.0); 7.457(0.9); 7.436(1.2); 7.421(1.5); 7.395(1.0); 7.374(4.0); 7.354(9.4); 7.339(10.5); 7.319(14.7); 7.288(12.6); 7.261(120.8);
7.247(16.1); 7.235(12.6); 7.212(1.0); 6.997(0.7); 6.809(5.5); 6.791(5.5); 5.299(0.6); 5.259(2.1); 5.237(6.2); 5.217(7.6); 5.197(5.8); 5.175(1.9); 4.498(3.1); 4.477(7.1); 4.460(7.0); 4.439(3.9); 4.425(1.6);
4.147(0.3); 4.131(0.4); 3.972(0.4); 3.956(0.5); 3.758(0.4); 3.731(0.5); 3.490(0.7); 3.403(2.3); 3.239(0.4); 3.224(0.6); 2.763(0.8); 2.754(1.5); 2.742(2.4); 2.713(6.0); 2.704(6.5); 2.695(5.4); 2.681(3.5);
2.673(3.2); 2.664(2.6); 2.633(2.3); 2.605(4.6); 2.591(7.2); 2.578(6.1); 2.558(4.4); 2.479(7.6); 2.473(8.2); 2.461(12.5); 2.453(12.5); 2.444(12.9); 2.421(4.4); 2.413(3.3); 2.350(0.4); 2.293(0.4); 2.218(0.3);
2.197(0.5); 2.171(0.5); 2.093(0.3); 2.044(1.3); 2.008(0.6); 1.988(0.5); 1.972(0.4); 1.885(0.5); 1.851(0.4); 1.828(0.5); 1.811(0.6); 1.792(0.6); 1.737(0.6); 1.574(31.5); 1.432(0.8); 1.414(1.5); 1.332(1.0);
1.293(2.3); 1.255(12.5); 1.110(0.3); 0.989(0.6); 0.971(1.0); 0.952(0.6); 0.880(1.7); 0.863(1.0); 0.843(0.8); 0.828(0.8); 0.146(0.5); 0.069(0.4); −0.001(101.0); −0.150(0.5)
Example 576: ¹H-NMR(399.8 MHz, cdcl3):
δ = 8.543(0.5); 8.512(0.5); 8.465(1.1); 8.453(13.2); 8.447(15.5); 8.400(16.0); 8.394(13.3); 8.237(0.4); 7.926(2.9); 7.908(3.0); 7.699(0.3); 7.519(1.1); 7.456(5.9); 7.434(7.3); 7.389(0.5); 7.330(0.6);
7.312(1.9); 7.291(7.0); 7.275(19.0); 7.260(183.9); 7.233(2.6); 7.178(0.3); 6.996(1.0); 5.201(1.0); 5.178(3.1); 5.158(3.9); 5.139(3.4); 5.117(1.2); 4.490(1.8); 4.470(4.4); 4.450(4.7); 4.428(1.9); 3.499(0.6);
3.486(0.5); 3.468(0.4); 2.743(2.1); 2.727(3.4); 2.719(2.7); 2.705(3.6); 2.698(3.9); 2.675(3.8); 2.654(3.6); 2.641(3.2); 2.604(1.1); 2.498(1.0); 2.477(2.6); 2.469(2.6); 2.454(4.4); 2.435(5.2); 2.424(5.9);
2.406(3.9); 1.895(0.3); 1.653(0.6); 1.601(1.6); 1.550(346.5); 1.503(1.0); 1.433(0.5); 1.412(0.3); 1.255(1.0); 1.207(0.4); 1.191(0.3); 0.146(0.9); −0.001(149.8); −0.150(0.8)
Example 577: ¹H-NMR(399.8 MHz, cdcl3):
δ = 12.664(0.5); 11.572(0.5); 8.769(0.8); 8.745(1.0); 8.735(1.1); 8.702(10.8); 8.692(11.1); 7.826(0.7); 7.806(0.9); 7.543(11.9); 7.523(16.0); 7.508(7.8); 7.490(15.4); 7.460(13.6); 7.449(13.8); 7.441(7.6);
7.429(7.5); 7.399(3.6); 7.379(7.6); 7.365(8.1); 7.346(4.9); 7.326(1.7); 7.300(9.4); 7.260(439.6); 7.218(1.2); 7.132(1.2); 6.996(2.6); 6.055(4.5); 6.040(4.8); 5.958(0.7); 5.300(5.2); 5.250(1.9); 5.227(5.6);
5.208(6.7); 5.188(5.2); 5.168(1.7); 4.498(2.3); 4.475(5.8); 4.453(6.6); 4.432(2.7); 4.150(0.5); 4.145(0.6); 4.130(0.6); 3.746(0.5); 3.709(0.8); 3.685(0.8); 3.639(0.5); 3.554(0.6); 3.486(1.1); 3.471(0.6);
2.724(4.5); 2.715(3.8); 2.701(4.4); 2.694(3.1); 2.681(1.8); 2.671(1.6); 2.606(1.4); 2.577(3.6); 2.562(5.1); 2.546(4.7); 2.534(4.3); 2.512(1.5); 2.492(2.2); 2.475(4.4); 2.451(5.4); 2.427(5.7); 2.411(5.3);
2.403(5.6); 2.385(4.7); 2.173(0.5); 2.159(0.6); 2.060(0.6); 1.997(0.5); 1.979(0.7); 1.949(0.6); 1.649(0.8); 1.598(2.2); 1.548(787.2); 1.473(0.6); 1.456(0.5); 1.420(2.1); 1.388(0.5); 1.293(0.7); 1.285(0.7);
1.255(2.6); 1.227(0.9); 1.208(1.1); 1.191(0.7); 1.165(0.6); 0.854(0.7); 0.146(2.0); 0.096(0.5); 0.079(0.6); 0.048(1.2); −0.001(376.8); −0.049(0.6); −0.129(0.8); −0.150(1.9)
Example 578: ¹H-NMR(400.0 MHz, CD3CN):
δ = 7.801(9.8); 7.800(10.0); 7.782(10.3); 7.780(10.4); 7.477(15.1); 7.471(15.8); 7.330(5.1); 7.328(5.1); 7.312(11.1); 7.309(10.9); 7.293(6.7); 7.290(6.8); 7.285(6.9); 7.280(6.3); 7.264(9.3); 7.259(9.0);
7.169(16.0); 7.148(11.3); 7.086(5.3); 7.082(5.7); 7.066(8.3); 7.063(8.8); 7.047(4.4); 7.043(4.5); 6.954(9.3); 6.950(9.0); 6.935(8.4); 6.931(7.9); 6.428(3.2); 5.447(1.6); 3.385(2.1); 3.373(4.2); 3.366(4.5);
3.354(8.3); 3.347(3.0); 3.343(4.6); 3.335(4.4); 3.324(2.2); 2.527(3.2); 2.508(7.0); 2.486(7.2); 2.468(3.3); 2.134(126.1); 2.113(1.7); 2.107(2.0); 2.101(1.4); 2.095(0.8); 1.964(11.2); 1.958(26.4); 1.952(130.4);
1.946(233.7); 1.940(310.9); 1.933(211.5); 1.927(107.6); 1.780(0.7); 1.774(1.3); 1.768(1.8); 1.762(1.2); 1.756(0.6); 1.462(4.1); 1.446(6.2); 1.443(5.9); 1.440(5.4); 1.426(6.4); 1.423(6.8); 1.420(5.2);
1.404(4.8); 1.340(0.4); 1.324(4.7); 1.312(5.5); 1.307(7.4); 1.295(7.4); 1.290(4.6); 1.279(4.2); 1.271(2.2); 0.881(0.4); 0.146(2.8); 0.008(26.1); 0.000(607.6); −0.009(22.2); −0.150(2.8)
Example 579: ¹H-NMR(400.0 MHz, CD3CN):
δ = 7.620(0.4); 7.525(8.2); 7.523(7.6); 7.506(9.9); 7.503(9.1); 7.481(14.8); 7.476(15.5); 7.354(0.4); 7.326(0.5); 7.312(3.2); 7.309(3.6); 7.294(9.5); 7.291(9.5); 7.282(7.0); 7.276(13.1); 7.272(8.0);
7.261(15.8); 7.256(16.0); 7.242(7.9); 7.237(8.3); 7.223(3.3); 7.218(2.9); 7.170(15.0); 7.149(10.3); 7.106(0.3); 7.085(0.3); 7.038(8.8); 7.034(9.0); 7.020(7.1); 7.015(7.3); 6.460(2.7); 5.447(2.4); 3.401(2.0);
3.389(4.1); 3.382(4.3); 3.378(2.8); 3.370(8.0); 3.363(3.0); 3.359(4.4); 3.351(4.4); 3.340(2.2); 2.517(3.0); 2.498(6.5); 2.476(6.7); 2.458(3.3); 2.149(434.7); 2.120(2.4); 2.113(2.7); 2.107(3.0); 2.101(2.1);
2.095(1.2); 1.964(12.9); 1.958(32.6); 1.952(169.5); 1.946(306.6); 1.940(409.3); 1.934(282.3); 1.928(146.3); 1.781(1.1); 1.774(1.9); 1.768(2.5); 1.762(1.8); 1.756(1.0); 1.689(0.3); 1.459(4.1); 1.442(6.2);
1.439(5.9); 1.436(5.1); 1.423(6.5); 1.420(6.7); 1.417(5.1); 1.401(4.9); 1.371(0.5); 1.340(1.1); 1.317(5.0); 1.305(5.8); 1.300(7.7); 1.288(8.1); 1.283(5.9); 1.271(8.5); 1.206(0.5); 1.190(0.4); 1.173(0.4);
1.130(0.4); 1.109(0.5); 0.881(1.3); 0.857(1.2); 0.841(0.8); 0.146(3.3); 0.008(27.7); 0.000(744.5); −0.009(32.0); −0.150(3.4)
Example 580: ¹H-NMR(400.0 MHz, CD3CN):
δ = 7.502(0.4); 7.483(13.6); 7.477(14.0); 7.446(0.4); 7.378(0.3); 7.346(1.6); 7.337(15.2); 7.335(16.0); 7.326(14.8); 7.321(9.4); 7.304(1.2); 7.301(1.4); 7.279(6.0); 7.272(6.8); 7.261(5.2); 7.258(11.5);
7.252(13.3); 7.243(4.6); 7.238(3.9); 7.230(3.4); 7.218(0.3); 7.170(13.4); 7.149(9.1); 7.102(9.5); 7.084(6.3); 7.082(6.1); 6.514(2.3); 5.447(3.3); 3.413(1.7); 3.402(3.7); 3.394(3.8); 3.390(2.5); 3.383(7.1);
3.375(2.5); 3.371(3.9); 3.364(3.8); 3.352(1.9); 2.514(2.8); 2.496(5.8); 2.474(6.2); 2.463(1.0); 2.455(3.0); 2.167(236.7); 2.161(408.6); 2.120(0.9); 2.113(1.2); 2.107(1.4); 2.101(1.0); 2.095(0.6); 1.964(7.8);
1.958(19.0); 1.952(93.5); 1.946(167.1); 1.940(222.0); 1.934(152.0); 1.928(77.9); 1.781(0.6); 1.774(0.9); 1.768(1.3); 1.762(0.9); 1.756(0.5); 1.458(3.5); 1.442(5.1); 1.439(4.9); 1.436(4.3); 1.423(5.3);
1.420(5.6); 1.417(4.4); 1.401(4.1); 1.340(0.6); 1.317(4.1); 1.305(4.7); 1.300(6.5); 1.288(6.5); 1.283(4.3); 1.271(4.4); 0.881(0.3); 0.146(1.6); 0.008(15.7); −0.001(332.3); −0.009(14.4); −0.150(1.5)
Example 581: ¹H-NMR(400.0 MHz, CD3CN):
δ = 7.669(5.4); 7.657(6.1); 7.654(5.6); 7.646(7.0); 7.636(1.0); 7.587(0.4); 7.552(1.8); 7.548(1.3); 7.539(12.6); 7.527(15.1); 7.517(11.7); 7.501(1.3); 7.492(15.2); 7.486(16.0); 7.303(6.8); 7.298(6.5);
7.282(9.1); 7.277(8.7); 7.164(15.3); 7.143(11.3); 7.029(0.7); 7.019(5.8); 7.009(6.0); 7.003(3.2); 6.997(5.5); 6.520(3.0); 5.448(0.8); 3.387(2.0); 3.376(4.1); 3.368(4.4); 3.357(8.1); 3.346(4.5); 3.338(4.3);
3.327(2.1); 2.518(3.2); 2.499(6.8); 2.477(7.2); 2.459(3.5); 2.166(730.1); 2.120(1.6); 2.114(2.0); 2.108(2.4); 2.102(1.7); 2.095(1.1); 1.965(10.7); 1.959(25.8); 1.953(135.6); 1.947(245.0); 1.940(328.1);
1.934(225.0); 1.928(115.8); 1.781(0.9); 1.775(1.5); 1.769(2.0); 1.763(1.4); 1.756(0.8); 1.458(4.1); 1.441(6.0); 1.439(5.9); 1.436(5.2); 1.422(6.0); 1.419(6.7); 1.417(5.4); 1.400(4.8); 1.340(0.6); 1.307(4.8);
1.296(5.4); 1.290(7.7); 1.279(8.0); 1.273(6.2); 1.262(4.2); 0.882(0.5); 0.859(0.4); 0.146(1.7); 0.008(13.4); 0.000(368.6); −0.009(14.5); −0.150(1.8)
Example 582: ¹H-NMR(400.0 MHz, CD3CN):
δ = 7.501(12.8); 7.496(13.2); 7.454(14.5); 7.441(15.5); 7.289(4.3); 7.284(3.9); 7.268(10.3); 7.263(10.2); 7.239(15.8); 7.218(6.4); 7.089(16.0); 7.076(14.9); 6.576(2.3); 3.406(1.8); 3.395(3.6); 3.387(4.4);
3.376(7.3); 3.365(4.3); 3.357(3.8); 3.346(1.9); 2.533(2.8); 2.514(6.0); 2.493(6.1); 2.474(2.9); 2.138(165.2); 2.120(1.6); 2.114(1.7); 2.108(2.0); 2.101(1.4); 2.095(0.8); 1.964(10.6); 1.958(25.4); 1.953(123.8);
1.946(221.5); 1.940(293.3); 1.934(201.6); 1.928(103.6); 1.781(0.7); 1.775(1.3); 1.769(1.7); 1.763(1.2); 1.756(0.6); 1.489(3.6); 1.473(5.8); 1.471(5.4); 1.467(4.3); 1.454(5.4); 1.450(6.0); 1.432(4.2);
1.341(0.3); 1.328(4.0); 1.317(4.6); 1.311(6.3); 1.300(6.3); 1.294(3.8); 1.283(3.4); 1.270(0.8); 0.454(0.5); 0.146(1.0); 0.008(9.1); 0.000(237.9); −0.009(9.6); −0.150(1.0)
Example 583: ¹H-NMR(400.0 MHz, CD3CN):
δ = 8.619(16.0); 8.615(15.8); 8.608(7.9); 8.604(6.9); 7.822(3.8); 7.818(5.7); 7.813(3.7); 7.802(4.2); 7.798(6.1); 7.793(3.8); 7.451(11.9); 7.446(12.1); 7.346(5.5); 7.333(5.5); 7.326(5.3); 7.314(4.7);
7.238(5.1); 7.233(4.8); 7.217(7.8); 7.212(7.4); 7.142(12.7); 7.121(8.1); 6.818(2.6); 4.068(0.7); 4.050(0.7); 3.415(1.6); 3.403(3.0); 3.396(3.6); 3.385(5.8); 3.374(3.6); 3.366(3.1); 3.355(1.7); 2.573(2.5);
2.555(5.6); 2.533(5.7); 2.514(2.6); 2.138(115.7); 2.114(2.4); 2.107(2.5); 2.101(1.8); 2.095(1.0); 1.972(4.8); 1.964(12.7); 1.958(31.9); 1.952(142.0); 1.946(251.2); 1.940(329.9); 1.934(227.0); 1.928(116.6);
1.781(0.8); 1.775(1.4); 1.769(1.9); 1.762(1.3); 1.756(0.6); 1.493(2.7); 1.476(5.0); 1.454(5.6); 1.435(3.9); 1.407(3.6); 1.395(4.6); 1.390(5.9); 1.378(5.8); 1.361(2.3); 1.222(0.8); 1.204(1.6); 1.186(0.8);
0.146(1.2); 0.000(249.9); −0.008(11.6); −0.150(1.2)
Example 584: ¹H-NMR(399.8 MHz, cdcl3):
δ = 7.681(0.4); 7.520(0.4); 7.489(4.1); 7.482(3.7); 7.471(4.5); 7.466(5.6); 7.403(0.3); 7.361(0.5); 7.347(1.0); 7.325(4.3); 7.316(5.8); 7.310(14.1); 7.306(16.0); 7.293(18.1); 7.285(6.0); 7.261(67.8);
7.117(0.9); 7.104(1.5); 7.090(2.3); 7.070(2.3); 7.043(14.1); 7.030(12.6); 6.997(0.5); 5.242(0.7); 5.221(2.3); 5.200(3.3); 5.181(2.5); 5.160(0.8); 4.465(1.5); 4.443(3.3); 4.425(2.8); 3.492(0.4); 2.741(0.7);
2.731(0.8); 2.710(2.0); 2.706(2.0); 2.700(2.2); 2.691(2.6); 2.681(2.5); 2.671(2.0); 2.663(2.0); 2.656(2.0); 2.649(1.4); 2.633(2.6); 2.618(2.7); 2.600(2.4); 2.578(1.3); 2.537(1.2); 2.532(1.3); 2.520(2.0);
2.514(2.2); 2.503(2.1); 2.497(2.7); 2.491(3.1); 2.481(3.1); 2.474(4.0); 2.467(3.8); 2.460(3.9); 2.444(2.2); 2.419(1.5); 2.401(0.4); 2.394(0.4); 2.035(1.9); 2.018(0.6); 1.928(0.8); 1.874(0.7); 1.255(0.6);
0.883(0.5); 0.147(0.4); 0.008(5.1); 0.000(75.8); −0.008(1.3)
Example 585: ¹H-NMR(399.8 MHz, cdcl3):
δ = 7.702(0.5); 7.681(0.6); 7.673(0.6); 7.652(0.8); 7.609(5.1); 7.600(5.7); 7.595(5.9); 7.586(6.6); 7.576(1.5); 7.552(1.1); 7.530(8.5); 7.519(2.7); 7.510(9.7); 7.482(2.8); 7.471(16.0); 7.463(12.2); 7.457(12.1);
7.449(14.2); 7.438(2.4); 7.421(1.3); 7.401(0.7); 7.382(2.4); 7.363(4.9); 7.349(5.1); 7.329(3.3); 7.314(1.2); 7.294(5.7); 7.260(100.6); 7.244(3.2); 7.091(1.1); 7.081(5.6); 7.072(5.6); 7.068(5.5); 7.058(4.8);
7.049(0.7); 6.996(0.5); 6.037(2.7); 6.017(2.8); 5.243(1.2); 5.221(3.5); 5.204(4.0); 5.182(3.1); 5.161(0.9); 4.486(1.4); 4.464(3.6); 4.443(4.2); 4.420(1.7); 4.138(0.4); 2.756(0.5); 2.741(1.0); 2.734(1.7);
2.725(2.1); 2.719(2.4); 2.711(3.0); 2.703(2.5); 2.697(2.4); 2.689(3.0); 2.680(2.1); 2.668(1.2); 2.658(1.1); 2.599(0.9); 2.571(2.4); 2.555(3.4); 2.541(3.0); 2.526(2.8); 2.501(1.0); 2.484(1.2); 2.477(1.3);
2.460(3.0); 2.453(2.5); 2.443(3.0); 2.436(3.7); 2.413(3.9); 2.406(3.8); 2.396(3.6); 2.388(4.0); 2.381(2.8); 2.371(3.0); 2.352(2.0); 2.343(1.5); 2.336(1.3); 2.324(0.6); 1.545(84.9); 1.258(0.6); 1.240(0.4);
1.046(0.4); 0.146(0.5); 0.000(108.1); −0.008(5.5); −0.150(0.6)
Example 586: ¹H-NMR(399.8 MHz, cdcl3):
δ = 7.714(5.6); 7.700(6.5); 7.693(6.6); 7.678(5.8); 7.645(0.4); 7.602(6.9); 7.585(8.4); 7.548(0.7); 7.503(2.8); 7.486(8.6); 7.471(14.8); 7.455(7.4); 7.437(2.2); 7.372(0.5); 7.316(6.9); 7.295(7.0); 7.260(31.3);
7.218(8.0); 7.201(6.9); 7.163(0.4); 7.133(0.3); 7.113(0.3); 7.069(3.5); 7.065(3.5); 7.048(6.3); 7.029(3.4); 6.996(0.4); 5.417(3.8); 5.403(4.0); 5.054(1.6); 5.035(4.6); 5.017(6.0); 4.999(4.5); 4.980(1.4);
4.398(2.1); 4.379(5.2); 4.360(5.0); 4.341(1.8); 2.713(1.4); 2.694(3.8); 2.677(4.5); 2.664(4.6); 2.646(4.3); 2.627(1.7); 2.404(6.7); 2.385(15.6); 2.365(16.0); 2.346(6.7); 2.186(2.7); 2.165(4.0); 2.150(3.9);
2.142(3.6); 2.134(3.6); 2.119(3.1); 2.098(1.2); 1.559(14.6); 1.256(3.2); 0.880(0.5); 0.861(0.4); 0.845(0.4); 0.070(3.2); 0.000(34.4)
Example 587: ¹H-NMR(399.8 MHz, cdcl3):
δ = 8.651(10.7); 8.647(11.2); 8.639(11.2); 8.635(10.6); 8.425(16.0); 8.421(15.8); 7.837(6.7); 7.833(10.1); 7.828(6.7); 7.818(7.3); 7.813(10.7); 7.808(6.7); 7.749(8.3); 7.735(8.8); 7.728(9.2); 7.714(8.6);
7.624(0.4); 7.520(0.8); 7.415(8.6); 7.409(8.8); 7.390(8.6); 7.384(8.6); 7.360(0.5); 7.312(9.5); 7.300(9.9); 7.293(9.4); 7.280(9.2); 7.261(120.5); 7.229(0.4); 7.144(4.6); 7.138(4.5); 7.122(7.8); 7.103(4.4);
7.098(3.9); 6.997(0.7); 5.669(4.4); 5.650(4.5); 5.121(1.8); 5.101(5.9); 5.082(8.3); 5.063(5.8); 5.043(1.7); 4.405(5.5); 4.389(5.5); 2.733(1.6); 2.730(1.7); 2.720(2.2); 2.713(3.4); 2.699(5.0); 2.689(4.3);
2.683(4.8); 2.679(5.3); 2.669(4.0); 2.664(4.7); 2.649(2.1); 2.645(2.0); 2.473(1.4); 2.452(3.4); 2.441(3.6); 2.431(3.8); 2.421(9.4); 2.409(5.2); 2.400(10.7); 2.379(8.2); 2.371(7.5); 2.355(4.8); 2.339(2.7);
2.325(1.3); 2.222(3.2); 2.200(5.1); 2.191(3.4); 2.183(5.1); 2.176(5.0); 2.159(2.7); 2.152(3.9); 2.130(1.8); 2.099(0.5); 2.056(0.3); 2.034(0.3); 2.005(0.3); 1.928(0.5); 1.692(0.3); 1.569(34.0); 1.333(0.4);
1.284(1.0); 1.255(5.3); 0.896(0.5); 0.880(0.8); 0.863(0.5); 0.146(0.7); 0.069(13.5); 0.000(135.8); −0.009(5.2); −0.150(0.6)
Example 588: ¹H-NMR(299.9 MHz, cdcl3):
δ = 7.697(4.1); 7.673(4.9); 7.606(1.8); 7.583(5.3); 7.564(13.6); 7.555(16.0); 7.523(9.0); 7.500(3.3); 7.262(21.1); 7.260(22.5); 7.101(1.7); 7.083(2.2); 7.064(2.4); 7.038(1.8); 6.024(2.1); 5.995(2.1);
5.040(0.8); 5.010(2.5); 4.981(3.6); 4.951(2.5); 4.921(0.7); 3.779(1.1); 3.748(2.7); 3.717(2.7); 3.686(1.0); 2.497(1.2); 2.468(2.9); 2.438(3.1); 2.409(1.5); 2.374(1.2); 2.343(2.9); 2.312(3.0); 2.282(1.2);
2.042(0.4); 1.991(1.0); 1.957(2.6); 1.925(3.8); 1.893(2.5); 1.860(1.1); 1.792(1.3); 1.757(2.9); 1.730(3.1); 1.723(2.8); 1.695(2.4); 1.660(0.7); 1.552(17.6); 1.549(19.0); 1.257(1.2); 0.001(22.8); 0.000(24.7)
Example 589: ¹H-NMR(299.9 MHz, cdcl3):
δ = 7.697(0.4); 7.638(0.5); 7.604(7.3); 7.578(11.2); 7.546(6.7); 7.526(8.4); 7.500(12.5); 7.481(14.3); 7.477(16.0); 7.454(7.5); 7.429(2.5); 7.401(0.6); 7.365(11.3); 7.339(8.6); 7.313(0.8); 7.284(10.0);
7.260(60.2); 7.256(47.6); 7.190(0.5); 7.133(5.3); 7.098(6.2); 7.068(4.6); 6.909(0.4); 5.402(3.6); 5.380(3.8); 5.053(1.3); 5.030(4.1); 5.006(5.5); 4.983(3.9); 4.958(1.2); 4.414(1.8); 4.390(4.7); 4.366(4.6);
4.340(1.7); 2.681(1.4); 2.657(3.3); 2.636(3.8); 2.632(4.2); 2.621(3.5); 2.617(3.8); 2.612(3.8); 2.608(3.1); 2.596(3.8); 2.592(4.4); 2.568(2.4); 2.519(1.4); 2.496(2.2); 2.478(3.2); 2.457(4.4); 2.446(3.8);
2.424(4.6); 2.400(3.9); 2.376(4.7); 2.371(4.8); 2.353(4.0); 2.348(5.3); 2.336(3.2); 2.331(2.8); 2.325(2.7); 2.321(2.5); 2.314(2.2); 2.308(2.7); 2.280(1.1); 2.147(2.5); 2.128(3.9); 2.107(4.2); 2.092(4.3);
2.075(3.4); 2.056(2.6); 2.044(0.9); 2.036(1.4); 1.550(39.9); 1.283(0.5); 1.255(2.4); 0.195(0.3); 0.067(0.4); 0.000(58.4); −0.004(44.7)
Example 590: ¹H-NMR(299.9 MHz, cdcl3):
δ = 7.857(7.1); 7.831(7.3); 7.634(3.8); 7.607(8.1); 7.578(2.9); 7.560(2.7); 7.552(3.8); 7.534(3.6); 7.508(1.5); 7.380(16.0); 7.367(10.7); 7.336(1.4); 7.327(0.9); 7.260(197.3); 7.191(0.6); 7.167(0.3);
7.134(0.7); 7.121(3.3); 7.108(3.6); 7.103(3.4); 7.093(6.3); 7.083(3.4); 7.076(3.2); 7.064(5.1); 7.029(2.6); 6.909(1.1); 6.001(2.2); 5.973(2.3); 5.067(0.8); 5.036(2.8); 5.006(4.0); 4.977(2.9); 4.946(0.8);
3.833(1.0); 3.799(2.8); 3.767(2.9); 3.734(1.1); 3.650(0.3); 3.524(0.4); 3.492(0.4); 3.482(0.4); 2.506(1.4); 2.476(3.0); 2.446(3.2); 2.417(1.6); 2.391(1.2); 2.360(3.0); 2.329(3.2); 2.298(1.5); 2.047(1.4);
2.013(3.1); 1.982(4.5); 1.951(2.8); 1.917(1.2); 1.794(1.5); 1.759(3.6); 1.732(3.5); 1.724(3.3); 1.697(2.8); 1.661(0.9); 1.607(0.8); 1.540(223.3); 1.473(0.7); 1.284(0.5); 1.259(0.8); 0.195(1.0); 0.067(0.9);
0.011(10.7); 0.000(216.0); −0.011(8.8); −0.069(0.6); −0.199(1.1)
Example 591: ¹H-NMR(299.9 MHz, cdcl3):
δ = 8.659(0.6); 8.646(0.5); 8.444(0.5); 8.417(0.7); 7.752(13.4); 7.729(16.0); 7.724(12.4); 7.643(0.4); 7.598(3.9); 7.572(9.8); 7.558(4.8); 7.541(3.9); 7.531(5.3); 7.524(3.3); 7.514(5.5); 7.499(8.6); 7.491(3.7);
7.480(5.2); 7.476(8.1); 7.448(13.7); 7.428(8.6); 7.423(15.6); 7.400(5.6); 7.395(4.0); 7.336(0.7); 7.328(0.8); 7.260(214.8); 7.193(0.6); 7.078(3.3); 7.040(3.6); 7.013(3.3); 6.908(1.1); 6.347(2.6); 6.318(2.7);
5.044(1.1); 5.013(3.5); 4.984(5.0); 4.955(3.5); 4.925(1.0); 4.133(0.4); 3.826(1.3); 3.793(3.8); 3.762(3.7); 3.731(1.3); 2.525(0.4); 2.499(1.8); 2.469(4.2); 2.437(4.2); 2.409(2.1); 2.375(1.6); 2.344(4.1);
2.314(4.4); 2.282(1.8); 2.044(1.2); 2.010(1.9); 1.975(4.5); 1.944(5.9); 1.914(3.7); 1.880(2.1); 1.821(2.4); 1.786(4.8); 1.759(4.8); 1.752(4.4); 1.725(4.1); 1.689(1.7); 1.668(1.0); 1.550(15.0); 1.420(1.0);
1.283(0.5); 1.259(1.2); 0.877(0.4); 0.856(0.4); 0.238(0.3); 0.195(1.3); 0.129(0.5); 0.098(0.7); 0.069(1.2); 0.010(13.8); −0.001(236.3); −0.011(5.9); −0.199(0.9)
Example 592: ¹H-NMR(299.9 MHz, cdcl3):
δ = 13.308(0.3); 7.657(1.7); 7.630(3.9); 7.612(3.9); 7.605(3.8); 7.586(2.3); 7.460(6.8); 7.433(6.6); 7.424(7.0); 7.400(11.3); 7.376(16.0); 7.340(11.7); 7.316(10.3); 7.288(4.6); 7.261(160.8); 7.197(3.7);
7.164(4.1); 7.132(2.9); 7.018(0.4); 6.909(0.9); 5.645(2.3); 5.594(0.4); 5.284(0.3); 5.108(0.9); 5.080(2.4); 5.056(3.7); 5.031(2.7); 5.005(0.8); 4.418(2.7); 4.398(2.7); 3.554(0.3); 2.687(0.8); 2.661(1.7);
2.618(2.4); 2.597(2.2); 2.574(1.2); 2.502(0.6); 2.463(2.1); 2.444(2.7); 2.423(3.0); 2.410(3.5); 2.390(4.8); 2.365(3.5); 2.340(1.6); 2.324(1.5); 2.295(0.6); 2.145(1.4); 2.121(2.3); 2.102(2.1); 2.084(2.7);
2.049(1.9); 2.027(0.8); 1.542(108.7); 1.441(0.4); 1.260(0.8); 0.195(0.9); 0.000(177.1); −0.072(0.8); −0.199(1.0)
Example 593: ¹H-NMR(299.9 MHz, cdcl3):
δ = 7.811(0.3); 7.632(11.7); 7.605(14.4); 7.574(2.7); 7.559(14.8); 7.556(14.3); 7.548(16.0); 7.522(5.1); 7.495(1.6); 7.416(10.8); 7.410(15.7); 7.320(8.7); 7.314(10.0); 7.293(7.7); 7.286(9.2); 7.260(130.6);
7.257(93.8); 7.216(0.5); 7.190(0.6); 7.178(0.4); 7.119(0.5); 7.093(2.8); 7.074(3.5); 7.056(4.3); 7.027(3.4); 6.909(0.9); 6.479(3.6); 6.449(3.6); 5.017(1.3); 4.988(4.3); 4.959(6.2); 4.929(4.5); 4.900(1.3);
3.821(1.8); 3.790(4.6); 3.759(4.8); 3.727(1.7); 3.518(0.3); 2.501(2.1); 2.472(5.1); 2.439(5.3); 2.412(2.7); 2.387(1.8); 2.355(4.8); 2.323(5.3); 2.294(2.1); 2.029(1.7); 1.994(5.0); 1.963(6.5); 1.928(4.6);
1.898(1.6); 1.815(1.9); 1.781(5.1); 1.747(5.3); 1.719(3.8); 1.683(1.0); 1.604(0.8); 1.541(127.9); 1.474(0.5); 1.258(0.6); 0.195(0.6); 0.063(0.8); 0.000(147.8); −0.003(105.8); −0.067(0.6); −0.200(0.7)
Example 594: ¹H-NMR(299.9 MHz, cdcl3):
δ = 7.606(2.7); 7.587(13.3); 7.559(16.0); 7.536(3.1); 7.422(8.3); 7.396(6.1); 7.349(0.4); 7.331(0.5); 7.278(11.0); 7.272(17.1); 7.260(105.3); 7.256(50.1); 7.232(7.7); 7.225(6.2); 7.194(0.5); 7.160(3.8);
7.124(4.6); 7.095(3.4); 6.908(0.5); 6.069(2.7); 6.048(2.7); 5.089(1.0); 5.064(3.0); 5.041(4.1); 5.018(3.0); 4.991(0.9); 4.408(3.3); 4.383(3.2); 2.690(1.0); 2.669(2.0); 2.665(2.3); 2.649(2.3); 2.645(2.7);
2.641(2.9); 2.636(2.6); 2.630(2.6); 2.625(2.8); 2.621(2.9); 2.616(2.2); 2.605(2.9); 2.601(3.0); 2.580(1.5); 2.576(1.7); 2.524(1.0); 2.503(1.4); 2.485(2.5); 2.463(3.3); 2.452(3.0); 2.431(3.5); 2.420(1.9);
2.415(2.4); 2.411(2.3); 2.392(3.9); 2.388(3.4); 2.364(4.2); 2.352(2.2); 2.341(1.8); 2.336(1.8); 2.324(2.0); 2.296(0.8); 2.158(1.8); 2.139(2.9); 2.119(2.9); 2.102(3.2); 2.086(2.4); 2.067(1.9); 2.044(1.2);
1.543(80.5); 1.259(0.4); 0.195(0.5); 0.064(0.4); 0.010(6.2); −0.001(106.1); −0.005(48.8); −0.011(4.9); −0.066(0.4); −0.200(0.6)
Example 595: ¹H-NMR(399.8 MHz, cdcl3):
δ = 8.024(0.6); 8.003(0.7); 7.648(1.0); 7.635(1.1); 7.628(2.1); 7.615(2.1); 7.608(1.4); 7.594(1.2); 7.519(0.6); 7.458(0.9); 7.448(3.5); 7.436(1.1); 7.428(2.9); 7.335(2.8); 7.334(2.8); 7.329(1.7); 7.319(2.6);
7.313(16.0); 7.302(15.8); 7.296(2.7); 7.286(1.6); 7.280(3.2); 7.260(101.5); 7.192(1.7); 7.165(2.0); 7.144(1.5); 6.996(0.5); 5.599(1.1); 5.581(1.2); 5.081(0.4); 5.061(1.3); 5.042(1.9); 5.024(1.3); 5.005(0.4);
4.414(1.3); 4.396(1.3); 2.666(0.4); 2.648(0.9); 2.634(1.1); 2.630(1.1); 2.626(1.1); 2.622(1.0); 2.616(1.2); 2.604(1.1); 2.600(1.1); 2.585(0.5); 2.582(0.6); 2.485(0.3); 2.469(0.6); 2.454(1.1); 2.439(1.4);
2.429(1.3); 2.414(1.5); 2.400(0.8); 2.387(1.8); 2.378(0.5); 2.365(1.9); 2.356(1.0); 2.344(0.8); 2.334(1.0); 2.313(0.3); 2.125(0.7); 2.109(1.2); 2.100(0.8); 2.095(0.9); 2.082(1.3); 2.079(1.3); 2.071(0.9);
2.061(0.7); 2.054(1.0); 2.044(0.5); 2.036(0.5); 1.544(13.5); 1.259(0.4); 0.145(0.4); 0.016(0.3); 0.014(0.4); 0.013(0.5); 0.012(0.6); 0.011(0.8); 0.007(3.5); 0.006(2.8); 0.005(3.1); −0.001(108.5); −0.009(4.4); −0.014
(1.1); −0.016(0.9); −0.017(0.8); −0.018(0.7); −0.019(0.6); −0.021(0.6); −0.022(0.5); −0.023(0.5); −0.024(0.4); −0.025(0.4); −0.027(0.4); −0.028(0.4); −0.029(0.3); −0.150(0.5)
Example 596: ¹H-NMR(399.8 MHz, cdcl3):
δ = 7.737(7.2); 7.733(12.5); 7.728(7.9); 7.611(6.9); 7.608(5.4); 7.592(8.0); 7.566(3.1); 7.550(16.0); 7.539(5.6); 7.532(5.1); 7.519(5.6); 7.512(1.7); 7.499(1.7); 7.484(3.9); 7.481(4.7); 7.479(4.7); 7.476(4.2);
7.464(5.7); 7.461(6.6); 7.459(6.9); 7.456(5.9); 7.384(8.9); 7.364(12.9); 7.345(5.4); 7.317(0.3); 7.260(120.3); 7.078(2.8); 7.061(2.9); 7.049(3.7); 7.030(3.1); 6.996(0.7); 6.310(3.0); 6.288(3.0); 5.003(1.0);
4.980(3.8); 4.958(5.3); 4.936(3.8); 4.914(1.1); 3.819(1.4); 3.795(3.9); 3.772(3.8); 3.748(1.4); 2.483(1.7); 2.461(4.3); 2.439(4.4); 2.416(2.1); 2.367(1.7); 2.343(4.2); 2.319(4.4); 2.297(1.8); 2.002(1.7);
1.976(4.5); 1.952(6.4); 1.930(3.9); 1.926(3.9); 1.903(1.9); 1.813(1.9); 1.792(2.5); 1.787(4.7); 1.766(4.8); 1.761(4.6); 1.740(3.9); 1.714(1.2); 1.545(47.0); 1.422(0.6); 0.146(0.6); 0.008(6.5); 0.000(132.0); −0.008
(5.9); −0.150(0.6)
Example 597: ¹H-NMR(399.8 MHz, cdcl3):
δ = 17.656(0.4); 14.847(0.4); 13.387(0.4); 7.661(2.8); 7.641(6.5); 7.627(6.3); 7.607(3.6); 7.520(1.9); 7.453(10.9); 7.434(9.1); 7.408(4.0); 7.402(7.6); 7.398(6.7); 7.389(5.4); 7.384(8.9); 7.380(8.3);
7.360(16.0); 7.276(8.1); 7.272(7.0); 7.260(320.5); 7.237(19.4); 7.233(17.8); 7.217(3.7); 7.213(5.0); 7.203(5.5); 7.176(6.2); 7.155(4.8); 6.996(1.9); 5.613(3.7); 5.596(3.8); 5.078(1.3); 5.061(4.2); 5.041(5.8);
5.023(4.1); 5.003(1.4); 4.422(4.3); 4.405(4.3); 4.363(0.4); 3.624(0.4); 3.575(0.5); 3.536(0.5); 3.525(0.5); 3.505(0.5); 2.673(1.3); 2.654(3.1); 2.636(3.7); 2.632(3.5); 2.627(3.4); 2.621(3.9); 2.610(3.5);
2.606(3.6); 2.590(1.8); 2.496(1.2); 2.481(2.0); 2.466(3.5); 2.450(4.4); 2.440(4.0); 2.425(4.6); 2.410(4.3); 2.391(5.6); 2.370(5.7); 2.361(2.9); 2.349(2.3); 2.339(2.8); 2.318(1.2); 2.125(2.1); 2.109(3.5);
2.094(3.2); 2.082(4.3); 2.070(3.0); 2.061(2.4); 2.054(3.0); 2.037(1.5); 1.677(0.4); 1.637(0.6); 1.630(0.6); 1.543(137.7); 1.378(0.4); 1.258(0.7); 0.146(1.6); 0.137(1.0); 0.025(1.6); 0.008(17.0); 0.000(371.9); −0.008
(17.6); −0.046(0.8); −0.080(0.4); −0.150(1.8); −0.964(0.4); −1.498(0.4)
Example 598: ¹H-NMR(399.8 MHz, cdcl3):
δ = 7.656(8.9); 7.652(8.5); 7.638(10.6); 7.633(10.2); 7.597(6.1); 7.578(13.8); 7.563(5.9); 7.550(5.3); 7.543(6.9); 7.530(6.7); 7.523(2.8); 7.510(2.5); 7.427(0.3); 7.402(3.6); 7.397(5.4); 7.382(12.2);
7.381(12.0); 7.378(16.0); 7.370(7.2); 7.357(11.4); 7.353(10.3); 7.338(6.2); 7.331(10.5); 7.326(8.1); 7.312(9.7); 7.308(9.3); 7.295(4.3); 7.290(3.8); 7.260(131.8); 7.081(4.8); 7.061(4.9); 7.054(5.3);
7.033(4.6); 6.996(0.7); 6.465(3.6); 6.444(3.6); 5.038(1.5); 5.015(5.3); 4.993(7.5); 4.971(5.2); 4.949(1.6); 3.811(1.9); 3.787(5.3); 3.764(5.3); 3.739(1.9); 2.494(2.4); 2.473(5.8); 2.451(6.0); 2.428(2.8);
2.377(2.2); 2.354(5.8); 2.330(6.0); 2.306(2.4); 2.015(2.3); 1.989(5.8); 1.965(8.8); 1.942(5.4); 1.939(5.2); 1.916(2.4); 1.789(2.6); 1.768(3.3); 1.762(6.6); 1.742(6.7); 1.736(6.4); 1.716(5.5); 1.710(2.6);
1.689(1.7); 1.545(87.2); 1.255(0.9); 0.146(0.7); 0.050(0.3); 0.007(7.5); 0.000(144.0); −0.008(5.9); −0.150(0.7)
Example 599: ¹H-NMR(299.9 MHz, cdcl3):
δ = 18.002(0.4); 8.059(0.7); 8.034(0.7); 7.967(0.8); 7.946(0.8); 7.942(1.0); 7.855(1.2); 7.830(1.2); 7.775(11.8); 7.748(12.7); 7.635(0.8); 7.606(2.5); 7.595(2.9); 7.569(6.6); 7.551(7.0); 7.524(4.3); 7.459(0.8);
7.433(1.5); 7.404(13.0); 7.378(10.9); 7.341(0.8); 7.325(1.6); 7.307(6.2); 7.282(15.3); 7.260(229.3); 7.257(181.1); 7.215(1.1); 7.194(2.3); 7.175(1.3); 7.135(14.0); 7.109(16.0); 7.080(5.6); 7.046(6.5);
7.021(10.4); 6.995(5.1); 6.908(1.2); 6.010(0.6); 5.456(3.8); 5.433(4.1); 5.081(1.6); 5.057(4.5); 5.034(6.1); 5.009(4.7); 4.983(1.3); 4.406(5.1); 4.381(4.7); 4.133(0.7); 4.111(0.7); 3.776(0.5); 3.738(0.5);
3.562(0.6); 3.492(0.7); 2.695(1.7); 2.670(3.4); 2.646(4.5); 2.631(4.0); 2.607(4.6); 2.583(2.5); 2.546(1.8); 2.507(3.4); 2.485(4.8); 2.452(5.0); 2.430(2.5); 2.408(2.1); 2.386(5.0); 2.358(5.8); 2.319(3.3);
2.293(1.5); 2.276(0.5); 2.266(0.5); 2.213(2.8); 2.193(4.7); 2.173(4.5); 2.157(4.7); 2.140(3.8); 2.122(2.5); 2.103(1.6); 2.045(2.4); 2.018(0.5); 1.980(0.8); 1.949(0.8); 1.917(0.5); 1.856(0.6); 1.757(1.1);
1.726(1.2); 1.545(55.2); 1.284(1.1); 1.260(1.7); 0.196(1.6); 0.157(0.6); 0.126(0.9); 0.075(1.4); 0.065(2.6); 0.000(246.5); −0.003(186.8); −0.199(0.9)
Example 600: ¹H-NMR(399.8 MHz, cdcl3):
δ = 8.033(2.3); 8.028(0.9); 8.016(0.8); 8.011(2.4); 7.697(2.1); 7.691(13.6); 7.686(5.5); 7.674(5.4); 7.669(15.6); 7.568(2.1); 7.549(9.7); 7.537(3.3); 7.530(3.3); 7.518(3.4); 7.511(1.3); 7.497(1.1); 7.461(2.6);
7.457(1.0); 7.444(0.9); 7.440(2.3); 7.417(2.5); 7.411(16.0); 7.406(5.8); 7.394(4.8); 7.389(13.1); 7.277(0.8); 7.260(100.6); 7.249(0.8); 7.248(0.7); 7.243(0.3); 7.075(2.0); 7.057(2.0); 7.047(2.5); 7.027(2.1);
6.996(0.6); 6.299(2.1); 6.279(2.1); 4.993(0.7); 4.970(2.5); 4.948(3.6); 4.926(2.5); 4.904(0.7); 3.812(0.9); 3.789(2.6); 3.766(2.6); 3.742(0.9); 2.484(1.2); 2.461(2.9); 2.440(3.0); 2.417(1.4); 2.359(1.1);
2.337(2.8); 2.313(3.0); 2.289(1.2); 2.045(0.4); 1.993(1.2); 1.967(3.1); 1.944(4.4); 1.921(2.7); 1.917(2.7); 1.894(1.4); 1.818(1.4); 1.798(1.7); 1.792(3.3); 1.772(3.3); 1.766(3.2); 1.745(2.8); 1.719(1.0);
1.555(3.0); 1.420(0.7); 1.408(0.4); 1.259(0.5); 0.146(0.5); 0.008(4.7); 0.000(107.9); −0.008(4.8); −0.150(0.5)
Example 601: ¹H-NMR(399.8 MHz, cdcl3):
δ = 7.602(8.3); 7.596(12.3); 7.589(7.1); 7.577(16.0); 7.559(7.9); 7.553(5.4); 7.539(4.6); 7.519(1.3); 7.424(12.9); 7.405(10.5); 7.358(0.8); 7.311(3.1); 7.307(3.5); 7.301(3.5); 7.287(11.5); 7.276(22.6);
7.270(27.2); 7.266(42.8); 7.260(189.4); 7.251(18.6); 7.246(13.4); 7.242(7.4); 7.235(5.3); 7.229(4.6); 7.144(6.3); 7.118(6.9); 7.096(5.4); 6.996(1.0); 6.074(4.1); 6.062(4.1); 5.099(1.6); 5.080(4.9); 5.062(6.8);
5.045(4.8); 5.026(1.6); 4.409(5.6); 4.389(5.4); 2.679(1.6); 2.663(3.9); 2.660(3.9); 2.648(4.2); 2.645(4.8); 2.642(4.8); 2.637(4.0); 2.633(4.6); 2.630(4.6); 2.627(4.6); 2.615(4.9); 2.612(4.5); 2.596(2.4);
2.512(1.8); 2.496(3.0); 2.482(4.1); 2.466(5.3); 2.457(4.9); 2.441(5.2); 2.425(2.6); 2.404(2.5); 2.400(2.5); 2.382(6.3); 2.379(4.6); 2.365(5.5); 2.361(6.5); 2.357(4.1); 2.352(4.4); 2.344(3.0); 2.340(2.9);
2.335(3.3); 2.331(3.7); 2.313(1.5); 2.310(1.5); 2.157(2.9); 2.141(4.7); 2.131(3.6); 2.126(5.0); 2.116(5.0); 2.114(5.0); 2.111(4.9); 2.102(4.4); 2.087(3.5); 2.070(2.0); 1.545(141.4); 1.255(0.9); 0.146(0.9); −0.001
(196.0); −0.150(0.9)
Example 602: ¹H-NMR(399.8 MHz, cdcl3):
δ = 8.706(12.8); 8.703(13.0); 8.485(0.4); 8.086(3.6); 8.061(16.0); 8.058(15.1); 7.987(0.5); 7.572(4.0); 7.554(13.2); 7.550(13.7); 7.537(5.5); 7.530(6.1); 7.517(6.2); 7.510(2.2); 7.497(2.2); 7.456(0.4);
7.435(0.3); 7.260(87.3); 7.069(3.6); 7.050(3.7); 7.041(4.5); 7.020(3.9); 6.996(0.6); 4.997(1.3); 4.974(4.5); 4.952(6.4); 4.930(4.6); 4.907(1.3); 4.130(0.8); 4.112(0.7); 3.886(1.7); 3.862(4.7); 3.840(4.6);
3.816(1.6); 2.470(2.2); 2.448(5.2); 2.427(5.2); 2.402(3.3); 2.371(5.0); 2.347(5.3); 2.324(2.1); 2.073(2.2); 2.050(4.4); 2.045(6.4); 2.024(7.5); 2.001(4.7); 1.997(4.4); 1.974(2.0); 1.814(2.3); 1.793(2.9);
1.787(5.8); 1.767(5.8); 1.761(5.7); 1.741(5.0); 1.735(2.4); 1.714(1.8); 1.594(6.4); 1.417(1.4); 1.353(0.5); 1.277(1.0); 1.259(2.0); 1.241(0.9); 0.146(0.5); 0.030(0.5); 0.007(5.3); −0.001(97.4); −0.009(3.3); −0.150
(0.4)
Example 603: ¹H-NMR(299.9 MHz, cdcl3):
δ = 8.488(16.0); 8.058(0.5); 7.985(15.6); 7.704(0.4); 7.632(2.8); 7.605(7.6); 7.587(6.8); 7.560(4.3); 7.459(12.8); 7.433(11.6); 7.397(4.2); 7.339(0.8); 7.328(1.9); 7.262(143.3); 7.260(199.2); 7.196(1.4);
7.161(5.9); 7.129(6.5); 7.096(4.8); 6.909(1.1); 5.077(1.4); 5.054(4.4); 5.027(6.0); 5.005(4.4); 4.977(1.3); 4.489(0.4); 4.416(4.8); 4.395(4.4); 3.515(0.4); 2.683(1.3); 2.658(3.1); 2.634(4.0); 2.625(3.6);
2.614(4.0); 2.596(4.0); 2.572(2.2); 2.544(1.4); 2.523(2.0); 2.502(3.7); 2.482(4.4); 2.470(4.4); 2.450(4.8); 2.431(4.3); 2.404(5.7); 2.380(5.8); 2.363(3.2); 2.352(2.6); 2.338(2.8); 2.312(1.2); 2.193(2.4);
2.171(4.0); 2.152(3.5); 2.134(4.6); 2.117(2.9); 2.100(2.7); 2.077(1.6); 2.057(0.4); 1.609(1.6); 1.541(211.2); 1.478(0.9); 1.467(0.4); 1.256(2.1); 0.879(0.5); 0.856(0.4); 0.195(1.1); 0.068(2.4); 0.001(156.9); −0.001
(219.1); −0.064(1.3); −0.199(1.1)
Example 604: ¹H-NMR(400.0 MHz, DMSO):
δ = 8.317(0.6); 7.952(2.1); 3.367(0.5); 3.330(306.7); 3.307(0.9); 2.891(16.0); 2.732(13.4); 2.689(15.9); 2.676(1.0); 2.671(1.2); 2.667(0.9); 2.511(76.3); 2.507(150.3); 2.502(193.3); 2.498(136.7);
2.493(63.9); 2.333(0.9); 2.329(1.2); 2.324(0.8); 1.139(0.6); 0.146(0.7); 0.008(6.3); 0.000(161.2); −0.009(5.9); −0.023(0.4); −0.150(0.7)
Example 605: ¹H-NMR(400.0 MHz, DMSO):
δ = 20.004(1.1); 10.288(1.1); 9.042(2.3); 9.020(2.4); 8.559(4.0); 8.551(3.8); 8.549(4.0); 8.521(6.6); 8.498(6.6); 8.476(1.6); 8.466(12.7); 8.463(12.9); 8.454(13.0); 8.451(12.4); 8.317(7.4); 8.156(3.8);
8.138(3.5); 8.135(3.8); 8.044(12.4); 8.041(12.2); 8.024(12.9); 8.020(12.4); 7.873(2.8); 7.852(3.7); 7.777(8.7); 7.757(11.2); 7.723(2.0); 7.704(3.4); 7.685(1.7); 7.674(3.0); 7.645(6.8); 7.634(11.7);
7.628(11.5); 7.616(12.0); 7.449(2.0); 7.443(4.2); 7.431(5.9); 7.423(4.1); 7.411(10.2); 7.391(9.8); 7.372(5.0); 7.365(12.3); 7.354(11.0); 7.345(11.2); 7.333(10.4); 5.378(1.1); 4.890(1.3); 4.866(4.5);
4.845(6.7); 4.827(4.7); 4.808(1.6); 4.180(5.4); 4.161(5.0); 3.817(1.8); 3.793(2.1); 3.778(1.1); 3.644(1.2); 3.594(1.2); 3.451(1.7); 3.426(1.7); 3.403(2.9); 3.394(4.8); 3.377(4.9); 3.332(4047.2); 3.184(1.1);
2.676(10.9); 2.671(14.2); 2.616(1.7); 2.561(4.5); 2.507(1889.9); 2.502(2409.3); 2.498(1773.2); 2.436(2.1); 2.415(1.3); 2.409(1.2); 2.329(16.0); 2.311(1.7); 2.275(1.5); 2.247(3.7); 2.235(5.3); 2.206(4.9);
2.190(2.5); 2.171(4.3); 2.148(5.2); 2.130(7.0); 2.120(4.9); 2.109(3.9); 2.085(2.8); 2.060(1.6); 1.707(1.8); 1.683(1.9); 1.660(1.4); 1.235(12.1); 0.855(1.6); 0.146(4.6); 0.008(54.9); 0.000(1146.5); −0.008
(48.5); −0.149(5.3); −3.347(1.1); −3.688(1.2)
Example 606: ¹H-NMR(400.0 MHz, DMSO):
δ = 20.010(2.1); 13.793(1.7); 8.317(4.4); 3.343(662.2); 3.331(4031.2); 3.280(4.4); 3.266(2.6); 3.237(2.3); 3.186(1.8); 2.971(1.9); 2.675(12.8); 2.671(15.0); 2.511(1425.7); 2.507(2073.0); 2.502(2352.7);
2.498(1632.7); 2.438(2.4); 2.367(2.3); 2.329(16.0); 0.146(6.4); 0.126(1.9); 0.011(172.0); 0.008(182.0); 0.000(1257.2); −0.149(5.8); −2.724(1.8)
Example 608: ¹H-NMR(400.0 MHz, CD3CN):
δ = 8.402(7.9); 8.399(8.1); 8.391(8.1); 8.388(7.9); 7.992(8.5); 7.988(8.3); 7.971(9.4); 7.968(9.2); 7.831(0.5); 7.820(0.4); 7.732(0.5); 7.711(0.6); 7.583(0.8); 7.489(0.4); 7.470(13.3); 7.465(13.4); 7.293(9.0);
7.281(8.9); 7.273(8.5); 7.261(8.3); 7.251(0.4); 7.229(5.3); 7.224(5.1); 7.208(10.7); 7.203(10.7); 7.162(16.0); 7.141(8.2); 7.094(0.3); 7.081(0.5); 7.072(0.5); 7.062(0.4); 6.899(0.4); 6.640(0.5); 6.618(0.5);
5.447(2.4); 3.403(1.9); 3.391(3.9); 3.384(4.1); 3.380(2.6); 3.372(7.8); 3.365(2.5); 3.361(4.2); 3.353(4.1); 3.342(2.0); 2.765(2.6); 2.752(2.6); 2.530(2.8); 2.511(6.0); 2.489(6.2); 2.470(3.0); 2.281(0.4);
2.250(0.4); 2.132(544.3); 2.120(5.5); 2.113(5.9); 2.107(6.8); 2.101(4.7); 2.095(2.5); 2.001(0.9); 1.964(33.4); 1.958(81.8); 1.952(431.1); 1.946(773.9); 1.940(1029.8); 1.933(701.5); 1.927(356.3); 1.915(5.0);
1.780(2.3); 1.774(4.2); 1.768(5.9); 1.762(4.0); 1.756(2.0); 1.495(4.0); 1.479(5.5); 1.476(5.2); 1.473(4.5); 1.460(5.5); 1.456(5.8); 1.454(4.8); 1.437(6.2); 1.340(1.3); 1.333(4.4); 1.321(5.0); 1.315(6.6);
1.304(6.6); 1.299(4.5); 1.287(4.8); 1.271(5.7); 1.204(0.4); 0.882(1.0); 0.858(0.6); 0.146(7.4); 0.008(61.2); 0.000(1654.2); −0.009(55.6); −0.043(0.7); −0.150(7.4)
Example 609: ¹H-NMR(400.0 MHz, CD3CN):
δ = 7.957(1.0); 7.949(0.9); 7.943(1.0); 7.934(1.2); 7.602(0.4); 7.591(2.7); 7.583(2.4); 7.577(2.3); 7.568(2.6); 7.558(0.3); 7.494(2.1); 7.489(2.3); 7.335(0.9); 7.330(0.9); 7.314(1.4); 7.309(1.4); 7.233(2.4);
7.212(1.6); 6.930(1.1); 6.922(1.0); 6.916(0.9); 6.908(1.0); 6.653(0.5); 3.334(0.6); 3.326(0.7); 3.315(1.1); 3.304(0.7); 3.296(0.6); 3.285(0.3); 3.235(16.0); 2.535(0.5); 2.516(1.0); 2.494(1.1); 2.476(0.5);
2.158(21.5); 2.156(21.0); 1.964(0.9); 1.958(2.3); 1.952(12.1); 1.946(21.9); 1.940(29.3); 1.934(20.2); 1.928(10.4); 1.473(0.6); 1.457(0.9); 1.454(0.8); 1.451(0.7); 1.438(0.9); 1.435(1.0); 1.416(0.7);
1.350(0.7); 1.338(0.8); 1.333(1.1); 1.321(1.0); 1.316(0.6); 1.305(0.5); 0.008(1.7); 0.000(46.0); −0.008(1.8)
Example 610: ¹H-NMR(400.0 MHz, CD3CN):
δ = 9.290(13.1); 8.517(16.0); 7.505(10.0); 7.500(10.2); 7.313(4.9); 7.308(4.7); 7.292(6.5); 7.287(6.3); 7.178(10.9); 7.157(8.0); 6.725(2.5); 3.419(1.4); 3.408(2.9); 3.400(3.4); 3.389(5.7); 3.378(3.4);
3.370(3.0); 3.359(1.5); 2.541(2.4); 2.522(5.3); 2.500(5.5); 2.481(2.4); 2.462(1.2); 2.458(0.9); 2.257(0.4); 2.245(0.6); 2.155(284.0); 2.120(2.5); 2.113(2.5); 2.107(2.6); 2.101(1.9); 2.095(1.2); 2.076(0.5);
2.063(0.3); 1.952(138.3); 1.946(233.5); 1.940(299.7); 1.934(207.8); 1.928(107.4); 1.781(0.8); 1.774(1.4); 1.768(1.7); 1.762(1.2); 1.756(0.6); 1.490(2.8); 1.472(5.1); 1.451(5.2); 1.432(3.2); 1.340(0.6);
1.321(3.5); 1.309(4.3); 1.304(5.9); 1.292(5.9); 1.287(4.2); 1.275(4.2); 1.270(3.7); 0.881(0.5); 0.859(0.4); 0.146(2.2); 0.000(424.2); −0.150(2.1)
Example 611: ¹H-NMR(400.0 MHz, CD3CN):
δ = 7.452(3.0); 7.447(3.1); 7.238(3.0); 7.234(3.0); 7.220(1.3); 7.215(1.2); 7.199(1.9); 7.194(1.8); 7.109(3.1); 7.088(2.2); 6.322(2.7); 6.317(2.6); 6.237(0.7); 3.332(0.4); 3.321(0.7); 3.313(0.9); 3.302(1.4);
3.291(0.9); 3.283(0.8); 3.272(0.4); 2.504(0.7); 2.486(1.5); 2.464(1.4); 2.446(0.7); 2.326(16.0); 2.133(13.2); 2.113(0.3); 2.107(0.4); 1.964(1.9); 1.958(4.8); 1.952(22.3); 1.946(39.6); 1.940(52.1); 1.933(35.8);
1.927(18.3); 1.440(0.8); 1.424(1.3); 1.421(1.1); 1.405(1.4); 1.402(1.4); 1.383(1.0); 1.337(0.9); 1.324(1.1); 1.320(1.5); 1.308(1.5); 1.291(0.7); 0.146(0.4); 0.008(4.6); 0.000(89.3); −0.008(3.5); −0.149(0.4)
Example 612: ¹H-NMR(400.0 MHz, CD3CN):
δ = 7.519(1.5); 7.461(3.5); 7.444(3.4); 7.438(3.5); 7.386(3.0); 7.254(1.5); 7.215(1.4); 7.210(1.4); 7.194(2.0); 7.189(2.0); 7.097(3.3); 7.076(2.3); 6.625(0.8); 3.938(16.0); 3.338(0.4); 3.327(0.8); 3.319(0.9);
3.316(0.7); 3.308(1.5); 3.300(0.7); 3.297(0.9); 3.289(0.8); 3.278(0.5); 2.538(0.7); 2.520(1.5); 2.497(1.6); 2.479(0.7); 2.135(10.9); 1.964(1.1); 1.958(2.7); 1.952(14.2); 1.946(25.9); 1.940(34.7); 1.934(23.9);
1.927(12.2); 1.457(0.9); 1.441(1.5); 1.438(1.3); 1.435(1.2); 1.422(1.5); 1.419(1.7); 1.416(1.2); 1.399(1.2); 1.366(1.0); 1.354(1.2); 1.349(1.6); 1.336(1.6); 1.332(1.0); 1.320(0.8); 1.285(0.3); 1.270(1.1);
0.008(2.0); 0.000(61.4); −0.009(2.3)
Example 613: ¹H-NMR(400.0 MHz, CD3CN):
δ = 7.481(12.6); 7.476(12.9); 7.418(12.6); 7.413(13.1); 7.294(7.8); 7.289(7.3); 7.276(6.9); 7.273(12.9); 7.268(9.7); 7.256(7.8); 7.251(7.4); 7.163(13.0); 7.142(9.0); 7.099(16.0); 7.079(12.9); 6.532(2.3);
5.447(1.2); 3.403(1.8); 3.391(3.6); 3.384(3.8); 3.380(2.4); 3.372(7.1); 3.365(2.6); 3.361(3.8); 3.353(3.8); 3.342(1.9); 2.511(2.6); 2.492(5.7); 2.470(5.9); 2.452(2.8); 2.148(129.7); 2.120(0.9); 2.113(1.1);
2.107(1.4); 2.101(1.0); 2.095(0.5); 1.972(1.4); 1.964(6.6); 1.958(16.7); 1.952(85.4); 1.946(154.3); 1.940(205.8); 1.934(141.1); 1.928(72.0); 1.781(0.5); 1.774(0.9); 1.768(1.2); 1.762(0.8); 1.756(0.4);
1.457(3.7); 1.441(5.6); 1.438(5.5); 1.435(4.6); 1.421(5.7); 1.418(6.0); 1.415(4.6); 1.399(4.3); 1.312(3.9); 1.300(4.6); 1.295(6.1); 1.283(6.2); 1.278(4.0); 1.266(3.5); 1.204(0.6); 1.186(0.3); 0.146(1.5);
0.008(11.9); 0.000(357.2); −0.009(13.5); −0.019(0.6); −0.021(0.5); −0.023(0.4); −0.028(0.3); −0.150(1.5)
Example 614: ¹H-NMR(400.0 MHz, CD3CN):
δ = 7.489(0.4); 7.483(0.4); 7.472(2.0); 7.467(2.1); 7.264(1.0); 7.259(1.0); 7.244(1.3); 7.239(1.3); 7.158(2.2); 7.137(1.9); 6.542(0.4); 4.587(2.1); 4.574(1.5); 4.571(1.5); 4.563(0.7); 4.559(2.3); 4.350(0.6);
3.703(1.4); 3.319(1.6); 3.317(1.6); 3.313(0.8); 3.307(1.5); 3.301(1.5); 3.299(1.5); 3.291(1.6); 3.289(1.7); 3.278(0.7); 3.267(1.2); 3.256(0.7); 3.248(0.7); 3.237(0.4); 2.559(0.5); 2.467(0.4); 2.448(0.9);
2.426(1.0); 2.407(0.5); 2.131(59.6); 2.119(1.1); 2.113(1.1); 2.107(1.2); 2.101(0.9); 2.095(0.5); 1.991(3.2); 1.971(1.0); 1.964(5.6); 1.958(13.4); 1.952(76.3); 1.946(138.5); 1.940(185.6); 1.933(127.9);
1.927(66.1); 1.808(16.0); 1.780(0.5); 1.774(0.8); 1.768(1.1); 1.762(0.8); 1.756(0.4); 1.431(0.6); 1.415(0.9); 1.412(0.9); 1.409(0.8); 1.396(0.9); 1.393(0.9); 1.390(0.8); 1.374(0.7); 1.270(0.5); 1.222(0.8);
1.210(0.7); 1.204(1.2); 1.193(1.0); 1.188(0.7); 1.177(0.5); 0.146(1.5); 0.008(10.9); 0.000(333.2); −0.009(11.7); −0.150(1.4)
Example 615: ¹H-NMR(400.0 MHz, CD3CN):
δ = 7.471(2.9); 7.466(3.0); 7.264(1.2); 7.259(1.2); 7.243(1.6); 7.238(1.6); 7.116(2.6); 7.095(2.0); 6.102(0.5); 4.197(1.5); 4.189(2.4); 4.186(2.2); 4.177(2.2); 4.175(2.5); 4.167(1.5); 3.231(0.4); 3.220(0.7);
3.212(0.8); 3.201(1.3); 3.190(0.8); 3.182(0.7); 3.171(0.4); 2.850(3.0); 2.839(2.7); 2.827(2.8); 2.443(0.6); 2.425(1.2); 2.403(1.2); 2.384(0.6); 2.135(10.8); 2.107(0.3); 1.972(1.2); 1.964(1.6); 1.958(3.8);
1.952(19.7); 1.946(35.8); 1.940(47.9); 1.933(32.9); 1.927(16.8); 1.787(16.0); 1.774(0.4); 1.395(0.8); 1.378(1.2); 1.376(1.1); 1.372(1.0); 1.359(1.2); 1.356(1.3); 1.353(1.0); 1.337(0.9); 1.243(0.8); 1.232(1.0);
1.226(1.3); 1.222(0.5); 1.215(1.3); 1.210(0.8); 1.204(0.7); 1.198(0.7); 0.146(0.3); 0.008(2.7); 0.000(79.3); −0.009(2.9); −0.150(0.3)
Example 616: ¹H-NMR(299.9 MHz, cdcl3):
δ = 8.463(13.4); 8.458(14.1); 8.448(14.1); 8.443(13.7); 8.184(3.4); 8.153(3.6); 8.004(0.4); 7.829(13.3); 7.824(13.4); 7.801(15.1); 7.796(14.2); 7.606(6.8); 7.581(12.8); 7.553(5.4); 7.535(4.9); 7.526(7.1);
7.508(6.8); 7.499(2.9); 7.482(2.7); 7.384(16.0); 7.369(15.4); 7.356(14.4); 7.341(13.8); 7.326(0.5); 7.310(0.3); 7.286(0.4); 7.285(0.4); 7.283(0.6); 7.281(0.6); 7.280(0.7); 7.276(1.3); 7.275(1.5); 7.273(1.7);
7.271(2.0); 7.270(2.4); 7.268(3.1); 7.259(160.5); 7.251(4.0); 7.249(3.0); 7.247(2.1); 7.246(1.5); 7.244(1.4); 7.242(1.3); 7.241(1.3); 7.239(1.0); 7.237(0.9); 7.236(1.0); 7.234(0.9); 7.231(0.9); 7.226(0.5);
7.222(0.5); 7.217(0.4); 7.212(0.4); 7.192(0.4); 7.159(0.3); 7.093(0.5); 7.063(4.9); 7.036(4.7); 7.026(5.2); 6.999(4.6); 6.908(0.8); 5.042(1.4); 5.012(4.8); 4.983(6.8); 4.953(5.1); 4.922(1.5); 3.883(1.8);
3.854(4.8); 3.823(4.7); 3.792(1.6); 3.502(0.5); 3.482(0.4); 2.720(0.7); 2.464(2.3); 2.434(5.9); 2.407(6.4); 2.400(8.3); 2.375(6.1); 2.371(6.1); 2.335(5.9); 2.303(2.4); 2.075(3.0); 2.044(5.0); 2.040(6.0);
2.010(8.6); 1.979(5.7); 1.944(2.1); 1.788(2.2); 1.761(3.6); 1.754(7.6); 1.727(7.4); 1.719(7.3); 1.692(5.8); 1.684(2.8); 1.656(1.8); 1.642(0.4); 1.547(153.3); 1.518(0.5); 1.255(1.4); 0.881(0.6); 0.850(0.4);
0.194(0.6); 0.094(0.4); 0.066(0.4); 0.062(0.4); 0.054(0.4); 0.049(0.5); 0.041(0.6); 0.034(0.6); 0.032(0.7); 0.029(0.8); 0.027(0.7); 0.024(1.1); 0.022(1.3); 0.021(1.3); 0.019(1.4); 0.017(1.7); 0.015(2.2);
0.014(2.6); 0.009(6.8); 0.007(5.1); −0.001(161.0); −0.008(3.6); −0.010(3.2); −0.012(4.8); −0.015(0.8); −0.017(0.6); −0.019(0.4); −0.020(0.4); −0.030(0.4); −0.200(0.6)
Example 617: ¹H-NMR(299.9 MHz, cdcl3):
δ = 8.234(15.1); 8.229(15.1); 8.219(15.6); 8.214(14.7); 8.000(0.7); 7.971(0.4); 7.827(0.4); 7.745(14.1); 7.741(13.6); 7.718(16.0); 7.713(14.8); 7.613(4.1); 7.602(3.3); 7.594(4.8); 7.586(9.2); 7.568(10.0);
7.559(8.4); 7.542(9.7); 7.520(4.5); 7.463(15.6); 7.438(10.6); 7.393(0.7); 7.345(0.7); 7.323(1.1); 7.280(18.9); 7.265(28.3); 7.256(278.2); 7.244(4.5); 7.242(4.5); 7.238(15.2); 7.222(0.5); 7.184(0.4);
7.132(6.5); 7.096(6.8); 7.068(5.5); 6.905(1.5); 5.073(1.7); 5.048(5.1); 5.024(7.3); 4.998(5.2); 4.974(1.8); 4.422(2.5); 4.397(5.5); 4.373(5.3); 2.659(1.6); 2.639(3.8); 2.636(3.7); 2.616(4.7); 2.611(5.1);
2.606(4.8); 2.601(4.5); 2.592(5.4); 2.587(3.5); 2.577(5.3); 2.573(5.0); 2.553(3.3); 2.548(2.9); 2.528(2.2); 2.508(2.8); 2.488(4.6); 2.468(5.7); 2.456(5.6); 2.435(6.2); 2.413(5.2); 2.385(7.7); 2.373(2.6);
2.356(7.8); 2.344(4.5); 2.333(3.4); 2.328(3.1); 2.316(3.9); 2.292(1.4); 2.288(1.5); 2.190(3.4); 2.167(5.6); 2.155(3.9); 2.148(4.1); 2.134(5.7); 2.131(5.7); 2.116(3.6); 2.107(3.1); 2.096(3.8); 2.073(1.9);
2.049(0.4); 1.716(0.4); 1.669(0.5); 1.630(0.5); 1.541(279.5); 1.468(0.8); 1.439(0.6); 1.327(0.6); 1.251(4.2); 1.070(0.4); 0.878(0.7); 0.848(1.0); 0.830(1.1); 0.191(1.2); 0.066(0.5); 0.006(13.5); 0.004(12.0); −0.005
(284.6); −0.015(12.1); −0.033(1.3); −0.043(0.8); −0.203(1.4)
Example 618: ¹H-NMR(399.8 MHz, cdcl3):
δ = 8.931(15.6); 8.717(10.7); 8.708(10.9); 8.058(9.6); 8.038(10.1); 7.775(10.4); 7.756(12.8); 7.661(0.4); 7.625(11.6); 7.607(16.0); 7.592(11.9); 7.573(6.0); 7.519(1.0); 7.379(8.1); 7.367(9.1); 7.360(9.4);
7.348(13.2); 7.331(11.8); 7.312(5.8); 7.260(137.6); 6.996(0.8); 6.374(5.0); 6.355(5.1); 4.939(1.9); 4.918(5.7); 4.897(8.3); 4.875(5.9); 4.853(1.7); 3.819(2.6); 3.797(7.1); 3.774(7.1); 3.751(2.6); 2.535(2.6);
2.513(6.5); 2.491(7.3); 2.471(4.1); 2.346(3.1); 2.325(7.2); 2.301(6.9); 2.279(2.9); 2.072(0.3); 2.025(2.4); 1.999(6.1); 1.975(8.8); 1.951(6.5); 1.928(5.3); 1.903(6.5); 1.883(6.8); 1.857(5.0); 1.831(1.5);
1.563(124.6); 1.285(0.5); 1.254(1.2); 0.146(0.7); −0.001(146.6); −0.150(0.7)
Example 619: ¹H-NMR(399.8 MHz, cdcl3):
δ = 14.275(0.4); 11.590(0.4); 10.720(0.4); 9.251(0.4); 8.628(8.2); 8.617(8.4); 8.343(12.4); 8.218(0.4); 7.904(0.4); 7.812(8.4); 7.796(6.8); 7.792(8.9); 7.751(10.6); 7.730(13.6); 7.721(16.0); 7.712(10.3);
7.693(10.3); 7.672(3.6); 7.613(0.5); 7.519(2.3); 7.477(5.7); 7.459(9.1); 7.440(4.5); 7.408(0.5); 7.361(0.7); 7.292(8.2); 7.279(10.2); 7.260(333.2); 7.177(0.4); 7.160(0.4); 7.078(0.4); 6.996(1.8); 5.699(0.5);
5.623(4.2); 5.600(1.3); 5.068(1.5); 5.049(4.9); 5.030(6.9); 5.011(5.0); 4.993(1.6); 4.453(2.2); 4.435(5.1); 4.417(5.0); 3.650(0.4); 3.623(0.4); 3.611(0.4); 3.552(0.6); 3.527(0.5); 3.499(0.5); 3.487(0.5);
3.419(0.4); 2.726(1.3); 2.710(2.9); 2.695(4.2); 2.685(3.7); 2.675(4.1); 2.663(3.6); 2.645(1.7); 2.489(1.6); 2.473(3.7); 2.458(5.3); 2.448(7.4); 2.429(9.0); 2.418(4.3); 2.407(7.2); 2.387(2.9); 2.377(2.6);
2.357(1.0); 2.225(2.3); 2.207(4.0); 2.194(3.3); 2.179(4.6); 2.151(2.7); 2.134(1.5); 2.070(0.6); 2.056(0.5); 2.045(0.4); 1.679(0.6); 1.646(0.8); 1.547(402.3); 1.460(0.5); 1.443(0.6); 1.416(1.3); 1.370(1.8);
1.333(0.7); 1.293(1.5); 1.285(2.9); 1.254(7.4); 1.241(0.9); 0.880(0.7); 0.847(0.5); 0.173(0.4); 0.145(1.8); 0.099(0.6); 0.072(0.7); 0.000(349.0); −0.059(0.8); −0.150(1.7)
Example 622: ¹H-NMR(399.8 MHz, cdcl3):
δ = 8.522(12.6); 8.517(16.0); 8.471(16.0); 8.466(14.4); 7.936(3.1); 7.915(3.2); 7.781(7.3); 7.762(8.9); 7.609(8.3); 7.602(6.0); 7.590(10.4); 7.584(9.8); 7.563(4.1); 7.520(0.7); 7.335(4.7); 7.316(7.9);
7.297(3.9); 7.261(90.5); 6.997(0.6); 4.976(1.2); 4.954(4.1); 4.932(5.9); 4.910(4.2); 4.887(1.2); 3.855(1.7); 3.831(4.8); 3.807(4.9); 3.783(1.8); 3.526(0.4); 3.510(0.4); 2.835(0.3); 2.502(1.8); 2.481(4.6);
2.458(4.9); 2.436(2.3); 2.364(1.7); 2.340(4.6); 2.316(5.0); 2.293(2.1); 2.057(1.7); 2.030(4.5); 2.007(6.6); 1.981(4.2); 1.958(1.8); 1.882(1.9); 1.856(4.6); 1.835(4.7); 1.830(4.9); 1.809(3.8); 1.783(1.2);
1.746(0.5); 1.739(0.7); 1.547(81.9); 1.255(1.1); 0.146(0.5); 0.000(96.5); −0.150(0.5)
Example 623: ¹H-NMR(399.8 MHz, cdcl3):
δ = 8.415(14.6); 8.409(15.2); 8.237(16.0); 8.231(14.9); 7.718(6.7); 7.699(10.8); 7.679(8.3); 7.659(9.8); 7.652(6.5); 7.632(8.5); 7.613(3.8); 7.519(1.0); 7.405(4.9); 7.386(8.1); 7.367(3.9); 7.260(148.2);
6.996(0.8); 5.023(1.1); 5.004(3.7); 4.986(5.2); 4.968(3.8); 4.948(1.3); 4.432(1.7); 4.414(4.3); 4.396(4.3); 4.376(1.6); 2.712(1.1); 2.694(2.5); 2.678(3.2); 2.670(3.1); 2.666(3.1); 2.662(3.3); 2.648(3.2);
2.629(1.6); 2.524(1.0); 2.509(1.8); 2.494(3.1); 2.478(4.0); 2.468(3.9); 2.453(4.1); 2.437(3.6); 2.416(4.7); 2.395(4.8); 2.385(2.8); 2.375(2.2); 2.364(2.4); 2.345(0.9); 2.239(2.0); 2.222(3.2); 2.207(3.2);
2.194(3.7); 2.182(2.6); 2.168(2.2); 2.151(1.1); 1.545(178.1); 1.256(1.3); 0.146(0.8); −0.001(157.3); −0.150(0.8)
Example 624: ¹H-NMR(600.1 MHz, CD3CN):
δ = 8.519(13.9); 8.517(13.8); 8.511(14.2); 8.509(13.6); 8.075(0.4); 8.073(0.4); 8.062(0.5); 8.059(0.5); 8.051(15.3); 8.049(14.9); 8.037(16.0); 8.035(15.3); 7.967(2.7); 7.965(2.8); 7.954(2.8); 7.952(2.7);
7.883(10.4); 7.870(11.7); 7.676(5.7); 7.663(11.0); 7.655(11.5); 7.643(12.3); 7.402(6.2); 7.390(10.7); 7.377(5.1); 7.364(0.4); 7.356(0.4); 7.350(0.6); 7.344(15.3); 7.336(15.1); 7.330(14.7); 7.323(14.4);
5.447(11.9); 4.906(1.6); 4.891(5.7); 4.876(8.3); 4.861(5.9); 4.846(1.7); 3.878(2.1); 3.862(5.9); 3.846(5.9); 3.831(2.1); 2.349(2.3); 2.348(2.4); 2.333(6.4); 2.320(6.8); 2.317(5.8); 2.306(2.5); 2.305(2.7);
2.303(2.9); 2.268(2.1); 2.253(5.8); 2.252(5.9); 2.237(6.5); 2.221(2.8); 2.139(93.8); 2.133(4.1); 2.117(6.2); 2.116(7.3); 2.100(10.2); 2.085(6.1); 2.083(5.4); 2.067(2.6); 2.055(0.4); 2.051(0.6); 2.047(0.4);
1.964(1.8); 1.956(4.7); 1.952(5.6); 1.948(30.9); 1.944(54.0); 1.940(79.1); 1.936(53.5); 1.932(27.1); 1.923(0.5); 1.912(0.4); 1.829(0.3); 1.825(0.5); 1.821(0.5); 1.817(2.6); 1.802(3.1); 1.799(6.8); 1.785(7.0);
1.782(6.9); 1.768(6.1); 1.764(2.7); 1.750(1.9); 1.341(0.6); 1.284(0.8); 1.269(0.9); 0.005(1.0); 0.000(31.1); −0.006(1.0)
Example 625: ¹H-NMR(399.8 MHz, cdcl3):
δ = 7.544(0.3); 7.537(0.9); 7.524(0.9); 7.517(0.8); 7.505(0.8); 7.490(1.8); 7.471(0.8); 7.260(11.1); 7.087(0.7); 7.067(0.7); 7.059(0.7); 7.039(0.6); 6.331(0.5); 6.313(0.5); 4.886(0.6); 4.864(0.9); 4.842(0.7);
3.773(0.7); 3.749(0.7); 2.721(16.0); 2.451(0.8); 2.429(0.8); 2.405(0.4); 2.351(0.8); 2.327(0.8); 2.304(0.3); 1.971(0.7); 1.948(1.1); 1.925(0.7); 1.922(0.7); 1.810(0.3); 1.789(0.4); 1.784(0.8); 1.763(0.8);
1.757(0.8); 1.737(0.7); 1.552(2.1); 0.007(0.7); −0.001(12.9); −0.008(0.4)
Example 626: ¹H-NMR(399.8 MHz, cdcl3):
δ = 7.626(0.4); 7.605(1.0); 7.592(1.0); 7.586(0.7); 7.572(0.5); 7.398(1.7); 7.378(1.4); 7.261(24.5); 7.180(0.8); 7.154(0.9); 7.132(0.7); 5.799(0.6); 4.953(0.6); 4.936(0.8); 4.918(0.6); 4.384(0.7); 4.365(0.7);
2.664(16.0); 2.634(0.6); 2.615(0.6); 2.603(0.6); 2.600(0.6); 2.585(0.6); 2.496(0.4); 2.481(0.5); 2.466(0.7); 2.457(0.6); 2.441(0.6); 2.425(0.3); 2.377(0.7); 2.356(0.8); 2.347(0.5); 2.326(0.5); 2.107(0.5);
2.092(0.6); 2.081(0.6); 2.068(0.6); 2.053(0.4); 2.045(0.6); 1.543(14.6); 1.259(0.4); 0.000(27.0)
Example 627: ¹H-NMR(399.8 MHz, cdcl3):
δ = 7.577(2.8); 7.558(8.1); 7.552(5.9); 7.538(3.6); 7.531(4.1); 7.519(4.7); 7.499(1.5); 7.407(12.7); 7.394(13.7); 7.260(134.2); 7.212(0.4); 7.139(16.0); 7.126(15.1); 7.074(2.7); 7.045(3.2); 7.025(2.8);
6.996(0.8); 5.048(0.9); 5.026(2.9); 5.004(4.1); 4.982(2.9); 4.959(0.9); 3.866(1.1); 3.843(3.0); 3.820(3.1); 3.796(1.1); 2.496(1.4); 2.472(3.3); 2.451(3.5); 2.426(1.6); 2.403(1.3); 2.380(3.3); 2.356(3.5);
2.332(1.5); 2.102(0.4); 2.066(1.5); 2.044(3.7); 2.040(3.7); 2.017(5.4); 1.994(3.2); 1.990(3.0); 1.967(1.4); 1.782(1.5); 1.761(2.0); 1.755(3.8); 1.735(3.9); 1.729(3.8); 1.708(3.3); 1.703(1.6); 1.682(1.1);
1.545(34.6); 1.419(0.4); 1.277(0.6); 1.259(1.2); 1.242(0.5); 0.146(0.7); 0.008(7.7); 0.000(153.0); −0.008(7.3); −0.150(0.7)
Example 628: ¹H-NMR(399.8 MHz, cdcl3):
δ = 7.615(2.0); 7.602(2.4); 7.595(4.9); 7.582(5.0); 7.575(3.5); 7.562(3.3); 7.519(0.8); 7.503(8.8); 7.483(5.8); 7.330(14.5); 7.317(15.7); 7.303(0.5); 7.260(93.0); 7.143(3.8); 7.117(4.3); 7.095(3.5);
7.012(16.0); 6.999(14.7); 6.676(2.5); 6.661(2.5); 5.098(1.0); 5.080(3.0); 5.062(4.3); 5.045(3.1); 5.026(0.9); 4.407(3.2); 4.389(3.1); 2.669(1.0); 2.665(1.0); 2.650(2.2); 2.648(2.3); 2.633(2.8); 2.629(2.8);
2.625(2.6); 2.621(2.6); 2.617(2.7); 2.614(2.8); 2.603(2.9); 2.600(2.7); 2.585(1.6); 2.581(1.4); 2.533(1.2); 2.517(1.8); 2.503(2.7); 2.488(3.2); 2.478(3.0); 2.463(3.2); 2.448(1.6); 2.421(1.5); 2.418(1.5);
2.400(4.0); 2.391(1.3); 2.388(1.4); 2.382(3.3); 2.379(4.0); 2.370(2.6); 2.361(1.6); 2.358(1.6); 2.351(2.0); 2.349(2.3); 2.331(0.9); 2.190(1.8); 2.173(2.8); 2.164(2.0); 2.158(2.8); 2.146(3.1); 2.134(2.3);
2.126(1.6); 2.119(2.1); 2.102(1.2); 2.045(0.5); 1.549(37.2); 1.255(1.1); 0.146(0.5); 0.069(0.8); 0.008(6.1); 0.000(107.2); −0.008(3.6); −0.150(0.4)
Example 629: ¹H-NMR(399.8 MHz, cdcl3):
δ = 8.075(5.8); 8.072(5.8); 8.055(6.5); 8.052(6.3); 7.684(3.0); 7.681(3.2); 7.665(7.5); 7.662(7.6); 7.647(5.6); 7.644(5.2); 7.599(5.2); 7.595(5.9); 7.583(8.7); 7.577(16.0); 7.571(12.6); 7.559(7.0); 7.556(3.9);
7.540(1.1); 7.519(0.7); 7.497(7.2); 7.493(6.9); 7.478(5.7); 7.475(5.4); 7.260(83.3); 7.109(0.5); 7.100(1.8); 7.089(3.2); 7.073(2.3); 7.055(2.1); 7.048(1.6); 6.996(0.6); 6.044(2.1); 6.022(2.2); 5.009(0.8);
4.986(2.8); 4.964(3.9); 4.942(2.8); 4.920(0.8); 3.794(1.0); 3.771(2.8); 3.748(2.8); 3.724(1.0); 2.525(1.3); 2.503(3.1); 2.482(3.2); 2.459(1.4); 2.374(1.1); 2.350(3.0); 2.328(3.1); 2.303(1.2); 2.014(1.4);
1.991(2.6); 1.987(3.4); 1.964(4.9); 1.961(2.6); 1.942(3.1); 1.938(2.9); 1.915(1.5); 1.809(1.5); 1.789(1.9); 1.783(3.6); 1.762(3.7); 1.757(3.5); 1.736(3.2); 1.730(1.5); 1.710(1.1); 1.551(19.3); 1.255(0.5);
0.146(0.4); 0.008(4.2); 0.000(93.6); −0.009(2.9); −0.150(0.4)
Example 630: ¹H-NMR(399.8 MHz, cdcl3):
δ = 8.216(0.6); 8.196(0.7); 7.980(9.7); 7.960(10.9); 7.757(0.4); 7.738(0.8); 7.720(0.4); 7.649(0.5); 7.633(0.8); 7.613(0.9); 7.588(7.1); 7.572(14.9); 7.570(16.0); 7.554(14.8); 7.533(4.3); 7.525(7.4);
7.521(8.6); 7.505(9.6); 7.502(9.9); 7.486(4.1); 7.483(4.0); 7.427(0.8); 7.407(0.7); 7.383(11.4); 7.363(9.4); 7.289(0.8); 7.261(178.9); 7.222(0.7); 7.200(11.2); 7.197(11.4); 7.181(9.8); 7.178(9.9); 7.144(5.5);
7.118(6.4); 7.096(5.0); 6.997(1.1); 5.564(0.3); 5.361(3.8); 5.343(4.1); 5.068(1.3); 5.049(4.3); 5.031(5.9); 5.013(4.2); 4.994(1.3); 4.420(1.7); 4.401(4.6); 4.381(4.6); 4.363(1.7); 4.201(0.4); 3.490(0.3);
2.702(1.3); 2.686(3.2); 2.668(4.0); 2.653(3.9); 2.637(4.0); 2.619(2.0); 2.511(1.3); 2.494(2.3); 2.480(3.5); 2.465(4.6); 2.456(4.1); 2.440(4.7); 2.423(2.4); 2.410(2.1); 2.392(5.4); 2.371(5.8); 2.362(3.5);
2.354(3.0); 2.341(3.3); 2.323(1.4); 2.299(0.3); 2.225(2.5); 2.210(4.0); 2.195(4.5); 2.182(4.5); 2.170(4.3); 2.155(3.4); 2.139(1.6); 2.044(1.5); 2.007(0.5); 1.556(80.9); 1.333(0.5); 1.285(0.9); 1.277(0.8);
1.256(3.0); 1.242(0.6); 0.881(0.4); 0.853(0.3); 0.146(1.1); 0.070(0.8); 0.000(203.6); −0.149(0.9)
Example 631: ¹H-NMR(399.8 MHz, cdcl3):
δ = 8.879(0.3); 8.773(0.4); 8.730(0.3); 8.654(10.5); 8.644(10.4); 8.485(15.2); 7.940(0.3); 7.877(11.3); 7.857(12.0); 7.658(4.5); 7.637(9.7); 7.624(9.7); 7.618(6.4); 7.604(5.5); 7.536(0.4); 7.519(1.4);
7.454(16.0); 7.435(13.1); 7.406(0.6); 7.382(0.7); 7.324(10.2); 7.312(10.8); 7.305(10.3); 7.292(9.8); 7.260(195.1); 7.199(8.0); 7.173(9.0); 7.151(7.0); 6.996(1.1); 5.750(0.4); 5.683(5.2); 5.665(5.3);
5.619(0.5); 5.111(1.9); 5.092(6.4); 5.073(8.9); 5.054(6.2); 5.035(1.9); 4.430(6.3); 4.414(6.4); 4.368(0.7); 4.351(0.4); 3.493(0.3); 2.685(1.9); 2.667(4.1); 2.653(5.4); 2.644(4.7); 2.635(5.7); 2.621(4.9);
2.603(2.6); 2.509(1.8); 2.494(3.2); 2.479(5.2); 2.464(6.6); 2.454(6.3); 2.439(6.7); 2.423(6.0); 2.401(8.0); 2.381(8.2); 2.371(4.7); 2.360(3.5); 2.351(4.1); 2.330(1.5); 2.147(3.2); 2.130(5.3); 2.115(4.9);
2.104(6.2); 2.090(4.3); 2.082(3.6); 2.076(4.3); 2.058(2.4); 2.033(0.4); 1.558(88.2); 1.458(0.7); 1.434(0.4); 1.415(0.4); 1.397(0.3); 1.369(0.4); 1.334(0.4); 1.255(2.6); 1.008(0.6); 0.990(0.9); 0.971(0.4);
0.880(0.5); 0.847(0.7); 0.835(0.7); 0.145(1.0); −0.001(219.3); −0.151(1.2)
Example 632: ¹H-NMR(299.9 MHz, cdcl3):
δ = 7.609(4.1); 7.586(16.0); 7.558(11.0); 7.548(5.8); 7.532(9.3); 7.507(8.3); 7.503(8.7); 7.375(3.7); 7.351(8.2); 7.326(5.3); 7.294(5.8); 7.288(5.9); 7.261(90.7); 7.243(4.2); 7.167(0.4); 7.093(3.1); 7.057(3.9);
7.029(3.1); 6.909(0.5); 6.234(2.8); 6.207(3.0); 5.059(1.1); 5.028(3.6); 4.999(5.2); 4.970(3.7); 4.939(1.1); 3.823(1.4); 3.792(3.8); 3.762(3.9); 3.729(1.4); 2.506(1.8); 2.475(4.3); 2.445(4.5); 2.417(2.2);
2.391(1.7); 2.360(4.2); 2.328(4.4); 2.297(1.8); 2.038(1.6); 2.004(3.9); 1.972(5.7); 1.941(3.7); 1.907(1.5); 1.797(1.8); 1.763(4.3); 1.735(4.6); 1.728(4.3); 1.701(3.6); 1.666(1.1); 1.541(64.6); 1.258(0.7);
0.853(0.3); 0.197(0.5); 0.001(97.0); −0.198(0.4)
Example 633: ¹H-NMR(399.8 MHz, cdcl3):
δ = 7.715(1.2); 7.696(1.6); 7.625(1.3); 7.604(1.9); 7.583(1.4); 7.563(0.6); 7.353(0.8); 7.334(1.4); 7.315(0.6); 7.260(16.1); 6.336(0.5); 6.318(0.5); 4.839(0.7); 4.818(1.0); 4.796(0.7); 3.752(0.8); 3.729(0.8);
2.709(16.0); 2.482(0.8); 2.461(0.8); 2.439(0.4); 2.348(0.3); 2.326(0.8); 2.302(0.8); 2.278(0.3); 1.973(0.8); 1.951(1.2); 1.928(0.7); 1.924(0.7); 1.902(0.4); 1.881(0.4); 1.856(0.8); 1.835(0.8); 1.830(0.7);
1.809(0.6); 1.552(6.9); 1.254(0.5); 0.007(1.2); −0.001(18.3); −0.009(0.5)
Example 634: ¹H-NMR(399.8 MHz, cdcl3):
δ = 7.721(1.3); 7.701(1.4); 7.669(0.4); 7.657(3.4); 7.650(1.9); 7.641(1.4); 7.442(0.5); 7.422(0.9); 7.408(0.5); 7.402(0.4); 7.260(40.6); 5.762(0.5); 4.924(0.5); 4.907(0.8); 4.888(0.5); 4.383(0.6); 4.365(0.6);
2.669(0.5); 2.647(16.0); 2.624(0.5); 2.621(0.5); 2.475(0.5); 2.459(0.6); 2.449(0.5); 2.434(0.6); 2.418(0.3); 2.392(0.7); 2.371(0.7); 2.362(0.4); 2.341(0.4); 2.181(0.5); 2.165(0.5); 2.153(0.5); 2.140(0.4);
2.125(0.3); 1.552(18.0); 0.008(2.6); 0.000(45.8); −0.008(1.8)
Example 635: ¹H-NMR(399.8 MHz, cdcl3):
δ = 7.773(8.0); 7.754(9.5); 7.694(0.3); 7.616(8.5); 7.597(14.1); 7.577(8.7); 7.558(4.5); 7.519(1.3); 7.438(0.4); 7.384(11.8); 7.371(12.7); 7.337(5.4); 7.317(8.8); 7.298(4.6); 7.260(187.0); 7.124(16.0);
7.111(14.7); 6.995(1.2); 5.299(0.6); 5.015(1.3); 4.993(4.4); 4.972(6.1); 4.950(4.3); 4.928(1.2); 3.956(0.5); 3.856(2.0); 3.831(5.3); 3.808(5.3); 3.786(1.9); 3.739(0.3); 3.549(0.4); 3.490(0.4); 3.481(0.4);
3.459(0.3); 2.525(2.2); 2.503(5.1); 2.480(5.3); 2.459(2.5); 2.376(2.0); 2.353(5.2); 2.328(5.3); 2.305(2.2); 2.221(0.4); 2.058(2.0); 2.032(5.0); 2.009(7.1); 1.984(4.7); 1.960(1.7); 1.869(0.4); 1.845(2.1);
1.819(5.1); 1.798(5.3); 1.772(4.2); 1.746(1.5); 1.678(0.4); 1.544(107.6); 1.451(0.5); 1.419(1.2); 1.332(0.4); 1.285(0.6); 1.256(1.9); 1.222(0.4); 1.203(0.3); 0.879(0.7); 0.844(0.7); 0.145(1.3); 0.068(1.5); −0.001
(217.1); −0.150(1.1)
Example 636: ¹H-NMR(399.8 MHz, cdcl3):
δ = 7.790(8.8); 7.770(11.2); 7.718(9.2); 7.699(10.2); 7.659(5.3); 7.640(9.4); 7.621(4.5); 7.520(0.7); 7.403(5.6); 7.383(9.4); 7.364(4.3); 7.301(15.3); 7.288(16.4); 7.261(102.1); 6.997(1.0); 6.981(16.0);
6.968(14.8); 6.658(3.6); 6.643(3.6); 5.059(1.3); 5.041(4.4); 5.023(6.2); 5.004(4.4); 4.985(1.3); 4.425(2.0); 4.407(4.7); 4.389(4.6); 4.369(1.9); 2.710(1.3); 2.692(2.7); 2.678(3.8); 2.670(3.4); 2.666(3.3);
2.660(4.0); 2.648(3.4); 2.629(1.7); 2.519(1.1); 2.505(2.0); 2.489(3.5); 2.475(4.5); 2.464(4.5); 2.449(4.8); 2.436(4.2); 2.417(5.8); 2.396(6.1); 2.387(3.2); 2.375(2.5); 2.366(3.0); 2.346(1.1); 2.267(2.4);
2.250(3.7); 2.238(3.0); 2.223(4.4); 2.196(2.8); 2.177(1.3); 1.549(113.8); 1.285(0.7); 1.255(2.0); 0.881(0.5); 0.831(0.4); 0.146(0.6); 0.000(114.2); −0.150(0.6)
Example 637: ¹H-NMR(399.8 MHz, cdcl3):
δ = 8.139(0.4); 8.118(0.4); 8.059(7.1); 8.039(7.7); 7.790(6.4); 7.770(7.7); 7.663(3.8); 7.644(10.0); 7.642(10.0); 7.634(13.5); 7.626(9.6); 7.615(16.0); 7.598(4.5); 7.581(5.5); 7.578(5.3); 7.561(7.4);
7.542(3.4); 7.519(0.8); 7.467(8.6); 7.448(7.0); 7.363(4.4); 7.344(7.0); 7.325(3.6); 7.303(1.0); 7.260(72.3); 7.028(0.4); 7.011(0.4); 6.996(0.6); 6.027(3.4); 6.008(3.3); 4.955(1.2); 4.934(3.7); 4.912(5.3);
4.890(3.7); 4.869(1.0); 3.774(1.8); 3.751(4.6); 3.727(4.8); 3.704(1.7); 2.591(0.4); 2.566(2.0); 2.544(4.3); 2.521(4.5); 2.500(2.1); 2.350(1.9); 2.326(4.5); 2.303(4.6); 2.280(1.8); 2.205(0.4); 2.187(0.4);
2.030(1.5); 2.004(3.9); 1.981(5.9); 1.957(3.8); 1.932(1.8); 1.892(2.0); 1.866(4.2); 1.846(4.6); 1.820(3.3); 1.794(0.9); 1.550(29.5); 1.256(0.6); 0.146(0.6); 0.069(0.7); −0.001(78.9)
Example 638: ¹H-NMR(399.8 MHz, cdcl3):
δ = 8.204(0.8); 8.183(0.9); 7.951(10.2); 7.948(9.2); 7.932(11.8); 7.928(11.5); 7.749(1.2); 7.728(11.6); 7.708(12.1); 7.692(2.5); 7.682(3.3); 7.658(6.4); 7.639(15.7); 7.624(8.9); 7.605(11.0); 7.587(4.3);
7.535(3.8); 7.532(4.8); 7.517(11.6); 7.513(12.9); 7.499(13.7); 7.495(16.0); 7.492(13.9); 7.477(11.3); 7.473(11.9); 7.458(4.8); 7.454(4.1); 7.436(0.6); 7.405(6.0); 7.386(9.8); 7.368(4.5); 7.260(127.9);
7.190(0.9); 7.172(0.8); 7.080(0.4); 7.058(11.9); 7.053(13.0); 7.039(9.3); 7.035(11.0); 6.996(0.7); 5.565(0.4); 5.540(0.4); 5.337(4.2); 5.321(4.4); 5.299(1.5); 5.038(1.7); 5.018(5.1); 5.000(6.9); 4.982(4.9);
4.963(1.5); 4.427(2.2); 4.408(5.5); 4.390(5.4); 4.371(2.1); 4.198(0.4); 4.016(0.4); 2.741(1.4); 2.737(1.5); 2.722(3.4); 2.719(3.3); 2.704(4.2); 2.701(4.3); 2.696(4.1); 2.692(4.0); 2.688(4.4); 2.686(4.6);
2.674(4.2); 2.670(4.0); 2.667(2.4); 2.655(2.1); 2.652(2.1); 2.500(1.2); 2.485(2.1); 2.470(4.1); 2.455(5.4); 2.444(5.5); 2.430(8.5); 2.412(8.9); 2.390(7.5); 2.381(3.6); 2.372(3.3); 2.369(3.2); 2.363(3.0);
2.360(3.4); 2.342(1.6); 2.339(1.6); 2.302(3.6); 2.285(5.2); 2.270(4.8); 2.258(5.2); 2.245(3.7); 2.239(2.9); 2.230(3.4); 2.212(1.9); 2.188(0.6); 2.165(0.5); 1.552(38.9); 1.386(0.3); 1.367(0.5); 1.255(1.1);
0.837(0.3); 0.146(0.6); 0.008(7.8); 0.000(143.5); −0.008(6.0); −0.150(0.6)
Example 639: ¹H-NMR(299.9 MHz, cdcl3):
δ = 7.607(0.7); 7.594(1.7); 7.576(2.0); 7.568(4.0); 7.550(4.0); 7.541(2.9); 7.523(2.5); 7.399(7.2); 7.373(5.4); 7.330(1.5); 7.309(3.0); 7.302(3.0); 7.288(2.1); 7.281(6.7); 7.274(3.7); 7.261(102.3); 7.232(2.1);
7.216(0.4); 7.167(0.5); 7.153(3.3); 7.116(3.7); 7.088(2.9); 6.949(0.4); 6.909(0.7); 6.875(1.4); 6.871(2.0); 6.862(10.0); 6.847(1.8); 6.835(16.0); 6.808(8.0); 6.799(2.0); 5.564(2.1); 5.136(0.8); 5.109(2.7);
5.085(3.7); 5.060(2.6); 5.036(0.9); 4.420(1.1); 4.396(2.7); 4.371(2.7); 2.695(0.8); 2.691(0.9); 2.670(1.9); 2.666(1.9); 2.650(2.3); 2.647(2.4); 2.642(2.5); 2.637(2.4); 2.631(2.3); 2.626(2.3); 2.622(2.7);
2.607(2.6); 2.602(2.6); 2.582(1.3); 2.578(1.4); 2.495(0.9); 2.474(1.2); 2.454(2.4); 2.434(3.1); 2.422(3.0); 2.410(2.9); 2.402(4.0); 2.381(5.6); 2.358(3.4); 2.354(4.3); 2.341(1.9); 2.330(1.7); 2.326(1.6);
2.313(1.6); 2.285(0.6); 2.171(1.6); 2.148(2.8); 2.129(2.6); 2.112(2.9); 2.109(2.8); 2.096(2.1); 2.088(1.5); 2.076(2.0); 2.054(1.1); 1.541(30.2); 1.256(1.6); 0.196(0.6); 0.070(0.4); 0.011(5.4); 0.000(113.5); −0.010
(5.4); −0.199(0.4)
Example 640: ¹H-NMR(299.9 MHz, cdcl3):
δ = 7.606(0.6); 7.580(2.2); 7.563(14.6); 7.552(11.7); 7.543(5.4); 7.526(4.1); 7.516(1.1); 7.500(1.3); 7.408(1.4); 7.387(3.0); 7.380(2.9); 7.366(2.1); 7.359(6.0); 7.352(2.3); 7.337(3.1); 7.331(3.6); 7.310(2.0);
7.259(66.4); 7.091(2.2); 7.071(2.6); 7.054(2.9); 7.028(2.4); 6.969(2.4); 6.961(9.8); 6.933(16.0); 6.906(8.1); 6.898(1.9); 6.833(0.4); 6.208(2.2); 6.181(2.2); 5.043(1.0); 5.012(3.2); 4.983(4.6); 4.953(3.2);
4.924(0.9); 3.805(1.2); 3.775(3.3); 3.744(3.4); 3.712(1.2); 2.508(1.5); 2.478(3.9); 2.450(4.0); 2.418(2.0); 2.382(1.5); 2.351(3.8); 2.320(4.0); 2.288(1.7); 2.043(0.7); 2.017(1.6); 1.982(3.9); 1.951(5.6);
1.921(3.4); 1.916(3.4); 1.886(1.7); 1.803(1.9); 1.775(2.3); 1.768(4.3); 1.741(4.4); 1.733(4.1); 1.706(3.6); 1.699(1.6); 1.671(1.2); 1.545(9.8); 1.424(1.0); 1.258(0.7); 1.234(0.3); 0.194(0.4); 0.008(4.2); −0.001
(74.7); −0.011(3.5); −0.200(0.3)
Example 641: ¹H-NMR(299.9 MHz, cdcl3):
δ = 11.750(0.4); 11.521(0.4); 8.814(0.6); 8.771(0.8); 8.756(0.7); 8.695(11.9); 8.682(11.8); 7.896(0.7); 7.874(0.5); 7.702(11.0); 7.676(13.5); 7.636(0.9); 7.606(2.4); 7.594(4.5); 7.567(10.2); 7.548(10.5);
7.540(7.6); 7.521(6.4); 7.499(13.2); 7.483(13.2); 7.473(10.9); 7.457(10.1); 7.419(1.0); 7.374(16.0); 7.348(12.4); 7.315(1.2); 7.261(187.3); 7.167(1.0); 7.150(7.5); 7.114(8.8); 7.085(6.8); 6.909(1.1);
5.533(0.5); 5.406(5.6); 5.383(6.0); 5.058(1.9); 5.033(5.6); 5.010(8.0); 4.985(6.0); 4.962(2.0); 4.402(6.7); 4.377(6.4); 4.133(0.8); 4.110(0.8); 3.744(0.8); 2.802(2.5); 2.694(1.9); 2.671(4.7); 2.649(5.8);
2.634(5.4); 2.625(5.3); 2.609(6.1); 2.585(3.2); 2.581(3.1); 2.537(2.2); 2.514(3.3); 2.496(5.0); 2.475(6.8); 2.464(6.2); 2.453(4.5); 2.443(7.3); 2.421(6.5); 2.393(8.0); 2.370(6.9); 2.365(8.1); 2.359(5.5);
2.353(5.1); 2.343(4.1); 2.337(3.7); 2.325(4.4); 2.301(1.9); 2.162(3.8); 2.141(5.9); 2.121(6.2); 2.105(6.0); 2.088(5.3); 2.069(4.2); 2.044(4.7); 1.980(0.5); 1.949(1.1); 1.920(0.4); 1.875(0.4); 1.852(0.8);
1.827(0.4); 1.794(0.5); 1.733(0.6); 1.691(0.4); 1.674(0.5); 1.544(61.5); 1.283(1.5); 1.259(2.9); 1.235(1.1); 0.878(0.5); 0.837(0.8); 0.196(1.0); 0.096(0.8); 0.070(0.5); 0.040(1.1); 0.000(206.0); −0.199(1.0)
Example 642: ¹H-NMR(299.9 MHz, cdcl3):
δ = 8.772(5.0); 8.768(5.4); 8.756(5.2); 8.752(5.2); 7.898(5.0); 7.894(5.1); 7.872(5.8); 7.869(5.9); 7.607(0.8); 7.595(1.3); 7.568(9.4); 7.553(11.7); 7.544(16.0); 7.534(15.0); 7.527(5.7); 7.356(0.3); 7.298(0.6);
7.288(0.8); 7.261(100.4); 7.114(2.1); 7.108(2.4); 7.079(2.5); 7.064(2.8); 7.045(1.6); 6.910(0.6); 6.038(2.4); 6.009(2.5); 5.014(1.0); 4.985(3.3); 4.956(4.4); 4.926(3.1); 4.896(0.9); 3.804(1.2); 3.772(3.3);
3.740(3.3); 3.709(1.1); 2.799(1.8); 2.514(1.4); 2.482(3.5); 2.455(3.6); 2.424(1.8); 2.386(1.4); 2.354(3.5); 2.324(3.7); 2.292(1.5); 2.044(0.4); 2.014(1.5); 1.979(3.8); 1.948(5.2); 1.918(3.0); 1.912(3.1);
1.883(1.8); 1.830(1.9); 1.804(2.1); 1.795(4.0); 1.769(3.9); 1.761(3.8); 1.734(3.2); 1.726(1.5); 1.699(1.0); 1.544(21.3); 1.431(0.7); 1.255(0.5); 0.196(0.5); 0.037(0.5); 0.027(0.7); 0.016(1.7); 0.011(4.6);
0.009(4.0); 0.000(109.4); −0.010(4.2); −0.019(0.4); −0.198(0.5)
Example 643: ¹H-NMR(299.9 MHz, cdcl3):
δ = 7.826(5.4); 7.606(0.4); 7.600(0.6); 7.581(0.7); 7.573(1.3); 7.554(1.4); 7.545(1.0); 7.527(0.8); 7.411(2.3); 7.385(1.7); 7.260(46.6); 7.138(1.1); 7.110(1.2); 7.103(1.2); 7.073(1.1); 6.647(1.7); 6.466(3.4);
6.285(1.7); 6.067(0.7); 5.048(0.8); 5.023(1.2); 4.998(0.8); 4.351(0.9); 4.329(0.9); 3.846(16.0); 2.615(0.6); 2.595(0.8); 2.591(0.8); 2.585(0.8); 2.580(0.8); 2.571(0.8); 2.556(0.8); 2.552(0.8); 2.532(0.5);
2.458(0.4); 2.438(0.8); 2.418(0.9); 2.406(0.9); 2.385(1.4); 2.356(1.3); 2.328(1.3); 2.316(0.6); 2.300(0.5); 2.287(0.6); 2.123(0.5); 2.099(0.8); 2.079(0.7); 2.063(0.9); 2.045(0.7); 2.028(0.6); 2.007(0.3);
1.549(14.5); 0.010(2.6); 0.000(48.4); −0.011(1.8)
Example 644: ¹H-NMR(299.9 MHz, cdcl3):
δ = 7.880(5.1); 7.607(0.4); 7.563(0.8); 7.537(3.9); 7.523(1.3); 7.512(1.4); 7.496(1.3); 7.469(0.5); 7.261(61.0); 7.066(0.7); 7.042(0.8); 7.028(0.9); 7.001(0.9); 6.991(2.0); 6.909(0.3); 6.810(3.9); 6.629(2.0);
6.562(0.7); 5.300(0.5); 4.937(0.9); 4.909(1.3); 4.880(0.9); 3.907(16.0); 3.775(0.4); 3.743(1.0); 3.713(1.0); 3.682(0.4); 2.805(0.6); 2.454(0.5); 2.425(1.2); 2.393(1.2); 2.366(1.3); 2.334(1.1); 2.301(1.1);
2.272(0.5); 1.985(0.5); 1.949(1.2); 1.919(1.7); 1.889(1.0); 1.884(1.0); 1.854(0.4); 1.776(0.5); 1.749(0.7); 1.742(1.3); 1.715(1.3); 1.707(1.3); 1.680(1.2); 1.644(0.4); 1.543(33.1); 1.257(0.7); 0.011(2.9);
0.000(66.8); −0.011(2.8)
Example 645: ¹H-NMR(299.9 MHz, cdcl3):
δ = 8.446(14.4); 8.438(15.8); 8.268(16.0); 8.260(14.9); 7.626(1.7); 7.607(2.7); 7.599(4.1); 7.581(4.1); 7.572(2.9); 7.554(2.8); 7.455(7.3); 7.428(5.1); 7.344(0.5); 7.260(150.3); 7.240(3.4); 7.155(3.7);
7.120(4.0); 7.091(3.0); 6.909(0.8); 5.087(0.9); 5.061(2.6); 5.036(3.5); 5.012(2.6); 4.986(0.8); 4.415(2.6); 4.392(2.6); 2.803(0.6); 2.680(0.9); 2.660(1.8); 2.636(2.3); 2.632(2.4); 2.627(2.1); 2.621(2.2);
2.612(2.5); 2.598(2.5); 2.593(2.4); 2.574(1.5); 2.538(0.9); 2.517(1.3); 2.497(2.3); 2.476(2.7); 2.464(2.7); 2.444(3.1); 2.428(2.5); 2.404(3.9); 2.388(1.4); 2.376(3.7); 2.364(1.8); 2.348(1.5); 2.336(1.8);
2.311(0.7); 2.192(1.5); 2.168(2.7); 2.150(2.1); 2.132(2.7); 2.118(1.5); 2.097(1.7); 2.074(1.0); 1.953(0.4); 1.623(0.5); 1.542(100.0); 1.252(0.5); 0.853(0.4); 0.195(1.0); 0.082(0.7); 0.010(8.6); 0.000(151.8); −0.011
(5.0); −0.199(0.5); −1.742(0.4)
Example 646: ¹H-NMR(299.9 MHz, cdcl3):
δ = 8.541(12.2); 8.533(15.5); 8.480(16.0); 8.473(12.9); 8.003(0.4); 7.948(2.1); 7.919(2.2); 7.606(0.7); 7.581(2.1); 7.560(13.8); 7.546(5.0); 7.536(4.3); 7.519(4.0); 7.493(1.4); 7.261(87.8); 7.228(0.5);
7.081(2.2); 7.059(2.4); 7.042(2.9); 7.016(2.4); 6.909(0.6); 5.031(0.8); 5.002(2.8); 4.971(4.2); 4.942(3.1); 4.911(0.9); 3.886(1.1); 3.853(3.0); 3.821(3.1); 3.789(1.1); 2.803(0.6); 2.483(1.3); 2.453(3.4);
2.425(3.5); 2.395(2.8); 2.372(3.3); 2.340(3.6); 2.311(1.4); 2.077(1.5); 2.041(3.6); 2.011(5.1); 1.980(3.2); 1.976(2.9); 1.945(1.3); 1.825(1.4); 1.797(2.1); 1.789(4.0); 1.762(3.8); 1.755(3.7); 1.727(3.2);
1.720(1.5); 1.692(1.0); 1.544(54.7); 1.417(0.7); 1.305(0.5); 1.283(0.4); 1.255(0.7); 0.195(0.5); 0.010(4.7); −0.001(92.3); −0.011(3.4); −0.199(0.4)
Example 647: ¹H-NMR(299.9 MHz, cdcl3):
δ = 8.473(0.9); 8.396(11.5); 8.390(13.1); 8.381(12.8); 8.374(12.7); 8.104(13.8); 8.098(13.6); 8.079(14.3); 8.072(14.2); 7.992(1.1); 7.630(3.5); 7.603(8.7); 7.585(8.6); 7.576(6.9); 7.558(5.7); 7.445(15.6);
7.419(11.6); 7.356(2.0); 7.310(16.0); 7.295(16.3); 7.285(17.7); 7.260(427.9); 7.197(1.6); 7.173(7.3); 7.138(8.0); 7.108(6.7); 7.071(1.3); 7.055(1.2); 6.909(2.7); 6.425(1.6); 6.386(4.8); 6.339(2.3); 5.104(1.6);
5.077(5.4); 5.053(7.2); 5.031(5.4); 5.005(2.1); 4.429(6.3); 4.405(6.4); 2.804(8.0); 2.704(1.7); 2.679(4.1); 2.654(5.3); 2.643(4.9); 2.635(5.5); 2.619(5.7); 2.594(3.2); 2.553(2.1); 2.515(4.5); 2.494(6.3);
2.483(5.8); 2.462(6.3); 2.438(5.3); 2.410(7.6); 2.387(6.2); 2.381(7.6); 2.370(4.8); 2.342(4.4); 2.317(1.7); 2.179(3.2); 2.159(5.3); 2.139(5.7); 2.123(6.0); 2.107(4.8); 2.088(4.3); 2.066(2.0); 1.951(0.9);
1.745(0.9); 1.628(1.5); 1.543(127.9); 1.441(1.0); 1.303(1.3); 1.254(2.0); 0.839(1.1); 0.196(2.2); 0.096(1.0); 0.010(24.8); 0.000(470.7); −0.075(1.0); −0.199(2.5)
Example 648: ¹H-NMR(299.9 MHz, cdcl3):
δ = 8.470(6.9); 8.463(7.5); 8.454(7.5); 8.447(7.3); 8.100(7.8); 8.094(7.7); 8.075(8.3); 8.069(7.9); 7.607(0.4); 7.560(11.2); 7.552(15.0); 7.544(16.0); 7.529(5.5); 7.502(1.3); 7.357(8.4); 7.341(8.4); 7.332(8.1);
7.316(7.6); 7.262(46.9); 7.101(2.5); 7.086(4.3); 7.065(3.4); 7.041(2.9); 7.033(2.4); 6.757(2.9); 6.729(2.8); 5.026(1.1); 4.996(3.9); 4.967(5.7); 4.938(3.9); 4.908(1.0); 3.856(1.5); 3.825(4.1); 3.794(4.1);
3.763(1.4); 2.799(1.4); 2.518(1.8); 2.487(4.4); 2.458(4.5); 2.426(2.3); 2.406(1.7); 2.375(4.3); 2.343(4.6); 2.312(1.9); 2.063(1.8); 2.043(1.0); 2.028(4.4); 1.997(6.4); 1.966(3.7); 1.962(3.8); 1.932(1.8);
1.840(2.1); 1.813(2.6); 1.806(4.9); 1.778(4.9); 1.771(4.7); 1.743(4.0); 1.708(1.2); 1.563(12.4); 1.426(1.1); 1.284(0.4); 1.259(0.5); 0.010(3.0); 0.000(46.2)
Example 649: ¹H-NMR(601.6 MHz, CD3CN):
δ = 7.748(0.3); 7.728(0.5); 7.726(0.7); 7.720(8.9); 7.715(9.6); 7.710(8.6); 7.706(15.5); 7.699(2.6); 7.698(2.4); 7.688(2.1); 7.687(2.5); 7.681(16.0); 7.676(7.9); 7.675(7.6); 7.672(9.1); 7.666(8.3); 7.660(0.6);
7.658(0.4); 7.413(10.9); 7.410(11.2); 6.958(5.0); 6.955(5.0); 6.944(5.7); 6.941(5.7); 6.732(10.8); 6.718(9.6); 6.299(1.7); 5.448(0.6); 3.938(1.2); 3.200(2.9); 3.192(3.2); 3.187(4.4); 3.180(4.4); 3.179(4.4);
3.175(3.4); 3.167(3.3); 2.848(2.3); 2.836(4.6); 2.833(2.9); 2.824(2.7); 2.821(4.8); 2.809(2.4); 2.148(9.9); 2.139(3.3); 2.135(5.8); 2.127(5.7); 2.123(3.3); 2.115(3.0); 1.967(0.4); 1.959(1.0); 1.954(1.1);
1.951(6.6); 1.946(11.6); 1.942(16.9); 1.938(11.4); 1.934(5.9); 1.660(3.4); 1.647(4.7); 1.645(4.1); 1.635(3.7); 1.6324(4.4); 1.6315(4.6); 1.619(3.1); 1.341(0.5); 1.283(0.7); 1.263(0.9); 1.217(0.4); 0.005(0.4);
0.000(13.2); −0.006(0.5)
Example 650: ¹H-NMR(600.1 MHz, DMF):
δ = 8.523(3.5); 8.517(3.5); 8.024(11.8); 7.880(5.9); 7.878(5.6); 7.867(6.9); 7.865(6.4); 7.699(2.5); 7.696(2.6); 7.692(0.4); 7.686(7.0); 7.684(6.6); 7.673(5.6); 7.671(4.9); 7.660(5.2); 7.657(5.4); 7.647(7.1);
7.645(6.6); 7.634(2.7); 7.632(2.3); 7.528(11.0); 7.525(11.2); 7.474(6.9); 7.472(6.8); 7.461(6.1); 7.460(5.9); 7.358(2.5); 7.355(2.0); 7.344(10.5); 7.341(11.0); 7.335(16.0); 7.321(3.5); 3.554(1.4); 3.546(2.9);
3.541(2.9); 3.538(2.2); 3.533(5.3); 3.528(2.3); 3.525(3.3); 3.520(3.3); 3.512(2.2); 3.482(8.1); 2.922(5.7); 2.918(11.5); 2.915(15.9); 2.912(11.2); 2.909(5.4); 2.751(6.1); 2.747(12.2); 2.744(17.2); 2.741(11.9);
2.738(5.9); 2.567(2.2); 2.555(4.9); 2.540(4.9); 2.527(2.3); 1.560(2.0); 1.550(4.0); 1.547(3.0); 1.545(2.6); 1.536(4.8); 1.535(4.6); 1.532(2.6); 1.525(3.8); 1.522(4.0); 1.517(4.5); 1.513(4.4); 1.505(4.5);
1.495(1.6); 1.378(0.3); 1.296(0.4); 1.279(0.4); 0.097(0.6); 0.005(5.9); 0.000(153.0); −0.006(5.4); −0.100(0.6)
Example 654: ¹H-NMR(299.9 MHz, cdcl3):
δ = 8.003(0.7); 7.607(1.7); 7.596(2.5); 7.569(5.7); 7.551(5.7); 7.542(4.1); 7.524(3.8); 7.481(7.7); 7.477(8.1); 7.455(9.7); 7.451(9.9); 7.412(14.0); 7.405(9.3); 7.386(16.0); 7.380(12.6); 7.344(0.8); 7.306(5.9);
7.302(6.5); 7.281(13.4); 7.277(13.4); 7.261(224.7); 7.224(7.5); 7.218(7.8); 7.198(8.2); 7.192(8.3); 7.173(4.0); 7.167(3.6); 7.146(4.7); 7.109(5.1); 7.081(3.7); 6.909(1.4); 5.802(2.9); 5.778(3.0); 5.099(1.2);
5.074(3.3); 5.050(4.8); 5.026(3.5); 4.999(1.0); 4.408(3.8); 4.384(3.8); 4.357(1.4); 4.157(0.5); 4.135(0.5); 4.110(0.5); 2.693(1.3); 2.672(2.8); 2.669(2.9); 2.658(1.8); 2.652(2.9); 2.647(3.6); 2.644(3.7);
2.633(3.4); 2.629(3.4); 2.624(3.4); 2.608(3.9); 2.604(3.7); 2.584(2.0); 2.580(2.1); 2.536(1.4); 2.514(2.1); 2.496(2.9); 2.475(3.8); 2.464(3.6); 2.442(4.1); 2.420(2.4); 2.410(2.2); 2.387(4.9); 2.382(3.6);
2.364(4.1); 2.359(5.1); 2.353(3.0); 2.347(2.9); 2.341(2.3); 2.336(2.2); 2.331(2.1); 2.324(2.2); 2.319(2.7); 2.291(1.2); 2.183(2.3); 2.163(4.0); 2.143(3.8); 2.127(3.9); 2.111(3.2); 2.091(2.5); 2.070(1.2);
2.045(2.1); 1.538(72.4); 1.428(1.0); 1.284(0.8); 1.260(1.8); 1.236(0.5); 0.874(0.3); 0.849(0.4); 0.824(0.3); 0.196(1.1); 0.104(0.4); 0.086(0.4); 0.071(0.4); 0.011(11.6); 0.001(258.8); −0.010(11.6); −0.198(1.0)
Example 655: ¹H-NMR(400.0 MHz, DMSO):
δ = 13.579(0.7); 8.918(6.5); 8.897(6.6); 8.317(1.0); 8.023(0.4); 8.017(0.4); 8.000(0.5); 7.954(0.4); 7.939(5.5); 7.934(5.8); 7.918(8.3); 7.913(9.4); 7.895(0.7); 7.875(0.8); 7.855(13.0); 7.833(8.7); 7.819(16.0);
7.814(14.9); 7.795(2.2); 7.778(1.3); 7.759(9.0); 7.739(12.2); 7.730(4.2); 7.711(9.3); 7.692(6.5); 7.646(6.5); 7.627(8.2); 7.608(3.2); 7.432(9.4); 7.413(8.3); 4.801(1.0); 4.779(4.0); 4.757(6.0); 4.736(4.2);
4.714(1.1); 3.872(2.1); 3.702(1.5); 3.679(4.2); 3.657(4.2); 3.633(1.5); 3.326(160.5); 2.676(1.3); 2.672(1.8); 2.667(1.3); 2.525(4.3); 2.511(99.3); 2.507(201.4); 2.502(266.4); 2.498(192.9); 2.493(92.8);
2.334(1.3); 2.329(1.8); 2.325(1.4); 2.259(1.8); 2.237(4.6); 2.215(5.0); 2.194(2.1); 2.162(1.4); 2.140(4.1); 2.117(5.3); 2.094(2.5); 2.074(2.0); 2.049(4.9); 2.037(1.5); 2.026(5.8); 2.002(3.5); 1.983(1.2);
1.978(1.2); 1.770(1.3); 1.744(4.1); 1.719(4.9); 1.697(3.5); 1.673(1.0); 1.398(1.5); 1.236(0.5); 1.182(0.3); 1.165(0.5); 1.141(1.4); 1.088(0.5); 1.071(0.6); 1.052(0.4); 0.146(0.6); 0.008(4.9); 0.000(142.4); −0.009
(5.0); −0.150(0.6)
Example 656: ¹H-NMR(400.0 MHz, DMSO):
δ = 8.548(1.0); 8.527(1.0); 7.931(0.9); 7.926(1.0); 7.910(1.1); 7.905(1.2); 7.826(2.4); 7.821(2.2); 7.702(1.9); 7.678(2.2); 7.639(0.5); 7.623(1.3); 7.605(1.2); 7.601(0.9); 7.592(1.1); 7.574(1.1); 7.556(0.4);
6.936(1.3); 6.918(1.3); 4.828(0.4); 4.808(0.7); 4.790(0.5); 4.126(0.6); 4.107(0.6); 3.326(41.2); 2.524(0.7); 2.511(14.2); 2.506(28.1); 2.502(36.9); 2.497(26.6); 2.493(13.0); 2.473(1.0); 2.458(0.8); 2.454(0.9);
2.434(0.6); 2.411(0.5); 2.239(0.4); 2.233(0.3); 2.225(0.5); 2.219(0.6); 2.213(0.4); 2.197(0.4); 2.192(0.4); 2.094(0.4); 2.086(0.5); 2.071(0.6); 2.057(0.5); 2.045(0.4); 1.398(16.0); 0.008(0.6); 0.000(18.6); −0.009
(0.6)
Example 657: ¹H-NMR(400.0 MHz, DMSO):
δ = 9.075(0.6); 9.054(0.6); 8.786(0.9); 8.775(0.9); 8.737(8.7); 8.727(8.6); 8.725(8.5); 8.674(6.9); 8.653(7.0); 8.317(0.9); 7.943(0.6); 7.922(7.6); 7.918(7.6); 7.901(8.2); 7.896(8.5); 7.862(1.4); 7.830(16.0);
7.825(15.5); 7.784(0.9); 7.772(0.9); 7.764(0.8); 7.752(0.7); 7.742(0.4); 7.727(7.0); 7.715(7.0); 7.707(8.3); 7.695(8.0); 7.672(11.4); 7.651(9.7); 7.463(9.3); 7.446(7.8); 7.444(7.9); 5.756(1.5); 4.838(2.9);
4.820(4.1); 4.803(3.0); 4.785(1.3); 4.763(0.5); 4.742(0.4); 4.154(1.5); 4.134(4.3); 4.115(4.2); 4.096(1.5); 3.664(0.4); 3.640(0.4); 3.327(233.9); 2.687(0.4); 2.676(1.5); 2.671(1.9); 2.667(1.4); 2.551(1.5);
2.511(108.4); 2.507(209.2); 2.502(272.1); 2.498(197.6); 2.493(98.3); 2.469(5.3); 2.450(3.2); 2.426(3.9); 2.401(3.2); 2.382(1.5); 2.334(1.4); 2.329(1.9); 2.325(1.4); 2.282(0.5); 2.273(1.1); 2.259(1.9);
2.253(2.9); 2.246(2.9); 2.238(3.5); 2.232(3.7); 2.225(3.4); 2.217(2.3); 2.210(2.6); 2.205(2.4); 2.188(1.0); 2.183(1.0); 2.154(0.6); 2.130(0.6); 2.118(0.9); 2.109(0.4); 2.086(2.8); 2.070(2.9); 2.056(3.2);
2.045(3.7); 2.031(3.5); 2.018(2.6); 2.007(1.8); 1.983(0.5); 1.763(0.4); 1.739(0.5); 1.731(0.9); 1.716(0.4); 1.398(0.7); 1.236(0.5); 1.141(1.4); 0.146(1.8); 0.008(16.7); 0.000(384.6); −0.008(16.4); −0.150(1.8)
Example 658: ¹H-NMR(400.0 MHz, DMSO):
δ = 9.075(6.8); 9.054(6.9); 8.789(7.9); 8.787(8.5); 8.777(8.2); 8.775(8.3); 8.316(1.1); 7.948(5.1); 7.943(5.5); 7.922(16.0); 7.902(9.5); 7.899(9.4); 7.862(13.2); 7.841(8.2); 7.825(15.7); 7.820(14.4);
7.784(8.6); 7.772(8.4); 7.764(6.7); 7.752(6.6); 5.757(0.7); 4.807(1.1); 4.785(4.0); 4.764(6.0); 4.742(4.1); 4.720(1.1); 3.686(1.5); 3.663(4.3); 3.640(4.3); 3.617(1.6); 3.327(356.3); 2.680(0.6); 2.676(1.4);
2.672(1.9); 2.667(1.3); 2.662(0.6); 2.525(4.6); 2.520(7.3); 2.512(105.2); 2.507(216.0); 2.502(287.3); 2.498(206.2); 2.493(97.2); 2.338(0.7); 2.334(1.4); 2.329(2.0); 2.325(1.4); 2.320(0.7); 2.284(1.7);
2.261(4.4); 2.239(4.9); 2.217(2.0); 2.177(1.4); 2.154(4.1); 2.131(4.8); 2.118(1.2); 2.108(2.1); 2.056(1.7); 2.031(4.6); 2.007(5.9); 1.996(1.8); 1.983(3.5); 1.959(1.3); 1.789(1.4); 1.764(4.1); 1.742(4.6);
1.738(4.6); 1.717(3.4); 1.691(1.0); 1.398(1.1); 1.141(1.3); 0.146(0.5); 0.008(4.5); 0.000(144.6); −0.009(4.5); −0.150(0.6)
Example 659: ¹H-NMR(400.0 MHz, DMSO):
δ = 8.990(0.4); 8.937(12.1); 8.931(14.8); 8.915(0.5); 8.898(0.6); 8.886(15.3); 8.881(12.0); 8.777(6.0); 8.755(6.0); 8.623(0.4); 8.617(0.4); 8.557(0.4); 8.551(0.4); 8.316(1.2); 8.293(0.4); 8.286(0.5);
8.185(0.5); 8.178(0.4); 7.909(0.4); 7.869(0.4); 7.864(0.5); 7.847(5.4); 7.842(6.7); 7.826(6.3); 7.821(8.8); 7.801(16.0); 7.796(11.9); 7.682(11.1); 7.661(8.7); 4.901(0.9); 4.884(2.6); 4.866(3.6); 4.848(2.7);
4.829(0.9); 4.279(0.4); 4.172(1.4); 4.152(4.0); 4.132(3.9); 4.112(1.4); 3.327(358.2); 2.680(0.7); 2.676(1.4); 2.671(2.0); 2.667(1.4); 2.662(0.7); 2.571(1.2); 2.553(2.9); 2.542(2.2); 2.533(4.4); 2.525(8.0);
2.520(9.6); 2.511(109.8); 2.507(222.0); 2.502(289.4); 2.498(205.0); 2.493(95.0); 2.476(2.4); 2.457(2.2); 2.449(2.2); 2.431(3.3); 2.405(2.8); 2.387(1.4); 2.338(0.8); 2.334(1.5); 2.329(2.0); 2.324(1.4);
2.320(0.7); 2.294(1.1); 2.289(1.2); 2.274(2.7); 2.268(2.4); 2.259(2.8); 2.253(3.2); 2.246(2.8); 2.241(2.0); 2.230(2.2); 2.225(2.1); 2.208(1.0); 2.204(0.9); 2.132(1.7); 2.118(2.8); 2.104(3.0); 2.092(3.3);
2.090(3.3); 2.079(2.9); 2.065(2.3); 2.052(1.3); 1.236(0.4); 0.812(1.2); 0.810(1.2); 0.796(1.2); 0.793(1.2); 0.146(0.6); 0.008(4.5); 0.000(145.5); −0.007(2.3); −0.009(4.4); −0.150(0.6)
Example 660: ¹H-NMR(400.0 MHz, DMSO):
δ = 9.292(6.4); 9.270(6.5); 9.001(12.5); 8.995(15.4); 8.950(16.0); 8.944(12.7); 8.317(1.4); 7.952(5.1); 7.947(5.5); 7.931(8.0); 7.926(8.7); 7.858(12.7); 7.837(8.3); 7.823(15.4); 7.818(14.3); 7.796(0.4);
7.790(0.4); 4.861(1.1); 4.839(4.1); 4.817(6.1); 4.796(4.2); 4.775(1.2); 3.751(1.5); 3.728(4.1); 3.705(4.3); 3.682(1.6); 3.326(350.3); 2.681(0.8); 2.676(1.8); 2.671(2.5); 2.667(1.8); 2.662(0.8); 2.525(6.1);
2.520(10.0); 2.511(135.8); 2.507(277.6); 2.502(369.7); 2.498(266.6); 2.493(126.8); 2.338(1.0); 2.334(1.9); 2.329(2.5); 2.325(1.9); 2.320(1.0); 2.286(2.1); 2.265(5.0); 2.243(5.2); 2.220(2.1); 2.193(1.4);
2.169(4.1); 2.147(5.1); 2.124(4.2); 2.099(5.3); 2.086(1.3); 2.076(6.0); 2.052(3.4); 2.028(1.1); 1.789(1.4); 1.763(4.3); 1.738(5.1); 1.717(3.5); 1.695(0.9); 1.398(0.6); 1.236(0.4); 1.140(0.6); 0.146(2.4);
0.030(0.3); 0.008(21.0); 0.000(585.5); −0.009(20.8); −0.150(2.5)
Example 661: ¹H-NMR(399.8 MHz, cdcl3):
δ = 8.196(0.4); 7.994(7.7); 7.991(7.6); 7.974(8.7); 7.971(8.4); 7.731(0.4); 7.605(3.6); 7.602(3.9); 7.586(9.6); 7.583(9.6); 7.568(6.9); 7.565(6.4); 7.535(6.2); 7.531(6.9); 7.515(7.4); 7.511(7.6); 7.496(3.8);
7.492(3.6); 7.476(0.7); 7.451(14.6); 7.446(15.4); 7.380(0.5); 7.363(2.1); 7.356(7.3); 7.335(16.0); 7.323(0.6); 7.308(11.4); 7.302(10.5); 7.287(5.0); 7.282(4.8); 7.260(174.4); 7.236(0.6); 7.232(0.5);
7.231(0.5); 7.224(0.4); 7.211(0.4); 7.188(8.8); 7.184(8.9); 7.169(7.8); 7.165(7.8); 6.996(1.0); 5.392(2.5); 5.372(2.7); 5.141(1.2); 5.122(3.4); 5.104(4.6); 5.085(3.3); 5.067(1.1); 4.292(1.6); 4.273(4.3);
4.254(4.3); 4.235(1.6); 4.088(0.3); 2.691(0.9); 2.687(0.9); 2.671(2.6); 2.661(1.4); 2.656(2.6); 2.652(2.9); 2.646(2.1); 2.642(2.7); 2.636(2.9); 2.632(1.6); 2.621(2.9); 2.617(1.5); 2.606(1.4); 2.602(1.3);
2.428(0.5); 2.413(0.9); 2.404(0.8); 2.398(2.9); 2.383(5.1); 2.374(4.3); 2.366(8.2); 2.358(6.0); 2.345(6.7); 2.335(1.8); 2.327(2.3); 2.325(2.2); 2.315(1.4); 2.294(0.6); 2.173(1.7); 2.157(2.9); 2.150(1.8);
2.142(2.8); 2.133(2.8); 2.130(2.8); 2.127(2.9); 2.118(2.3); 2.112(1.7); 2.109(1.8); 2.104(2.2); 2.088(1.1); 2.056(0.3); 2.044(0.9); 1.544(40.7); 1.333(0.4); 1.294(0.4); 1.285(0.7); 1.277(0.7); 1.255(3.4);
1.241(0.6); 0.880(0.5); 0.146(0.8); 0.069(0.7); 0.008(6.9); 0.000(196.3); −0.008(7.6); −0.023(0.6); −0.025(0.5); −0.032(0.4); −0.150(0.9)
Example 662: ¹H-NMR(399.8 MHz, cdcl3):
δ = 8.956(8.5); 8.951(8.4); 8.735(5.7); 8.731(6.0); 8.723(6.0); 8.719(5.7); 8.107(4.3); 8.102(6.1); 8.097(4.3); 8.087(4.7); 8.082(6.3); 8.078(4.3); 7.529(10.0); 7.519(1.0); 7.508(11.6); 7.400(5.2); 7.399(5.1);
7.388(5.3); 7.387(5.2); 7.381(5.1); 7.379(4.9); 7.368(4.8); 7.367(4.7); 7.349(14.8); 7.344(16.0); 7.306(0.4); 7.280(8.3); 7.275(7.7); 7.260(102.2); 7.254(7.5); 6.996(0.5); 6.448(2.5); 6.428(2.6); 4.875(1.0);
4.853(3.4); 4.831(4.9); 4.809(3.5); 4.787(1.0); 3.778(2.1); 3.754(5.0); 3.731(5.1); 3.707(2.1); 2.499(1.4); 2.477(3.5); 2.456(3.6); 2.432(1.8); 2.412(1.5); 2.388(3.8); 2.364(4.1); 2.341(1.7); 2.055(1.7);
2.031(3.2); 2.028(4.1); 2.005(6.0); 1.982(3.6); 1.978(3.4); 1.955(1.6); 1.797(1.9); 1.777(2.3); 1.771(4.8); 1.751(4.7); 1.745(4.6); 1.724(4.0); 1.718(1.9); 1.698(1.3); 1.567(24.6); 1.253(0.5); 0.146(0.4);
0.008(4.0); −0.001(104.5); −0.009(4.2); −0.150(0.5)
Example 663: ¹H-NMR(399.8 MHz, cdcl3):
δ = 8.455(0.4); 8.449(0.4); 8.401(8.2); 8.396(8.7); 8.389(8.6); 8.384(8.4); 8.302(0.4); 8.054(9.4); 8.049(9.4); 8.035(10.1); 8.030(9.5); 7.519(0.9); 7.419(14.6); 7.414(16.0); 7.390(7.6); 7.369(14.7);
7.357(0.9); 7.330(10.9); 7.325(9.7); 7.309(6.0); 7.305(13.9); 7.293(10.3); 7.286(9.9); 7.274(10.1); 7.260(152.0); 7.240(0.4); 6.996(0.8); 6.322(2.2); 6.308(2.2); 5.117(0.9); 5.099(3.0); 5.081(4.2); 5.062(3.0);
5.045(1.0); 4.307(1.6); 4.288(4.4); 4.269(4.3); 4.250(1.6); 2.678(0.9); 2.675(1.0); 2.663(1.4); 2.659(2.1); 2.657(2.2); 2.653(1.6); 2.648(1.4); 2.644(2.4); 2.641(2.6); 2.638(2.6); 2.633(2.1); 2.629(2.6);
2.627(2.6); 2.623(2.8); 2.619(1.7); 2.614(1.8); 2.611(2.8); 2.608(2.7); 2.605(1.5); 2.593(1.5); 2.589(1.4); 2.471(0.9); 2.455(1.4); 2.447(1.2); 2.440(2.5); 2.430(2.0); 2.425(3.3); 2.416(2.8); 2.408(1.8);
2.400(3.8); 2.393(2.0); 2.384(2.1); 2.379(2.4); 2.375(4.5); 2.371(3.1); 2.366(1.2); 2.358(3.4); 2.354(4.4); 2.350(2.2); 2.345(2.2); 2.341(1.6); 2.337(1.8); 2.333(1.7); 2.328(1.6); 2.324(2.0); 2.307(0.8);
2.303(0.7); 2.171(0.4); 2.131(1.6); 2.114(2.7); 2.106(1.7); 2.104(1.7); 2.099(2.8); 2.090(2.7); 2.087(2.6); 2.084(2.8); 2.075(2.4); 2.070(1.6); 2.067(1.6); 2.060(2.2); 2.045(1.1); 1.548(112.6); 1.415(0.7);
1.370(0.4); 1.333(0.7); 1.294(1.2); 1.284(1.4); 1.254(7.1); 0.880(0.9); 0.862(0.4); 0.146(0.6); 0.008(6.2); 0.000(169.5); −0.009(6.0); −0.150(0.7)
Example 664: ¹H-NMR(399.8 MHz, cdcl3):
δ = 9.002(0.4); 8.955(5.0); 8.733(4.0); 8.668(0.4); 8.656(0.4); 8.528(0.3); 8.116(0.4); 8.088(6.0); 8.068(6.4); 8.034(0.9); 7.767(0.3); 7.635(0.4); 7.573(2.7); 7.558(16.0); 7.541(5.4); 7.529(4.7); 7.520(2.3);
7.509(1.6); 7.444(0.4); 7.402(4.4); 7.390(4.7); 7.382(4.7); 7.370(4.0); 7.355(0.6); 7.337(0.7); 7.261(191.4); 7.260(192.6); 7.221(0.3); 7.086(3.0); 7.069(3.1); 7.057(3.5); 7.037(3.0); 6.996(1.1); 6.366(2.8);
6.345(2.9); 5.299(0.8); 5.014(1.1); 4.991(3.7); 4.970(5.2); 4.947(3.7); 4.925(1.0); 3.847(1.4); 3.823(3.8); 3.799(3.9); 3.777(1.3); 3.549(0.4); 3.532(0.6); 3.518(0.6); 3.498(0.3); 2.858(0.4); 2.503(1.9);
2.481(4.3); 2.459(4.4); 2.437(2.0); 2.378(1.7); 2.355(4.3); 2.330(4.4); 2.308(1.8); 2.101(0.8); 2.070(0.4); 2.057(0.4); 2.030(1.8); 2.004(4.3); 1.981(6.5); 1.958(3.9); 1.932(1.8); 1.907(0.4); 1.844(2.1);
1.817(4.8); 1.797(4.8); 1.792(4.4); 1.771(4.0); 1.744(1.5); 1.725(1.2); 1.717(1.2); 1.671(1.1); 1.559(23.9); 1.487(0.9); 1.422(0.3); 1.286(0.6); 1.255(2.2); 0.849(0.7); 0.184(0.3); 0.146(1.3); 0.119(0.4);
0.007(14.5); 0.000(213.3); −0.001(205.6); −0.008(6.0); −0.150(0.9)
Example 665: ¹H-NMR(399.8 MHz, cdcl3):
δ = 7.728(6.7); 7.707(8.3); 7.590(14.4); 7.582(8.8); 7.577(11.2); 7.569(16.0); 7.553(6.0); 7.547(4.4); 7.532(4.4); 7.526(3.7); 7.505(0.9); 7.392(12.5); 7.387(13.5); 7.290(9.7); 7.285(9.0); 7.275(0.9);
7.269(8.8); 7.264(8.1); 7.246(116.1); 7.227(0.5); 7.225(0.4); 7.224(0.4); 7.223(0.4); 7.222(0.4); 7.221(0.3); 7.217(0.4); 7.212(0.3); 6.982(0.7); 6.458(2.4); 6.438(2.4); 4.928(0.9); 4.906(3.1); 4.884(4.5);
4.862(3.1); 4.840(0.9); 3.721(1.3); 3.698(3.4); 3.674(3.4); 3.651(1.3); 2.491(1.3); 2.469(3.4); 2.448(3.5); 2.424(1.6); 2.330(1.3); 2.307(3.2); 2.283(3.5); 2.259(1.4); 2.006(1.5); 1.982(2.8); 1.979(3.7);
1.956(5.2); 1.933(3.4); 1.929(3.1); 1.906(1.6); 1.815(1.5); 1.794(1.9); 1.788(3.6); 1.768(3.7); 1.762(3.5); 1.742(3.0); 1.736(1.4); 1.715(0.9); 1.525(76.7); 1.497(0.7); 1.494(0.7); 1.492(0.6); 0.886(0.5);
0.868(1.1); 0.850(0.5); 0.132(0.6); −0.006(6.4); −0.014(136.0); −0.023(4.0); −0.164(0.5)
Example 666: ¹H-NMR(399.8 MHz, cdcl3):
δ = 7.743(0.4); 7.703(0.5); 7.678(12.6); 7.673(13.2); 7.629(6.8); 7.608(16.0); 7.586(11.1); 7.581(9.7); 7.565(4.6); 7.559(10.2); 7.557(8.6); 7.555(7.6); 7.553(6.3); 7.541(7.0); 7.539(7.7); 7.537(7.7);
7.535(7.1); 7.519(2.6); 7.390(0.5); 7.369(0.6); 7.360(0.6); 7.349(0.5); 7.316(2.1); 7.312(2.2); 7.307(1.3); 7.300(2.3); 7.296(5.1); 7.292(7.6); 7.289(5.5); 7.280(16.7); 7.276(23.1); 7.272(23.9); 7.270(22.1);
7.267(23.1); 7.260(457.0); 7.250(12.9); 7.245(7.2); 7.244(7.3); 7.234(4.4); 7.228(3.9); 7.207(0.4); 6.996(2.5); 6.028(2.8); 5.299(1.4); 5.076(1.3); 5.057(4.0); 5.039(5.5); 5.021(4.3); 5.001(1.4); 4.373(1.6);
4.356(4.1); 4.335(4.1); 4.316(1.5); 3.531(0.5); 3.506(0.4); 3.467(0.3); 3.428(0.3); 2.718(1.2); 2.714(1.2); 2.699(3.7); 2.695(2.5); 2.688(1.7); 2.683(3.1); 2.679(3.8); 2.672(2.2); 2.668(3.5); 2.663(3.5);
2.659(1.9); 2.648(3.4); 2.644(1.8); 2.632(1.7); 2.629(1.6); 2.578(0.4); 2.458(0.4); 2.442(0.8); 2.426(4.3); 2.422(3.9); 2.410(5.8); 2.403(13.5); 2.395(3.9); 2.383(9.7); 2.370(4.2); 2.363(3.1); 2.351(1.0);
2.331(0.5); 2.206(2.1); 2.188(3.5); 2.181(2.3); 2.173(3.0); 2.163(3.2); 2.160(3.3); 2.157(2.9); 2.152(2.5); 2.141(1.9); 2.134(2.4); 2.116(1.2); 2.099(0.5); 2.071(0.7); 2.056(0.6); 2.006(0.4); 1.646(0.6);
1.613(0.4); 1.596(0.6); 1.545(459.7); 1.517(32.7); 1.515(36.2); 1.512(31.6); 1.271(0.6); 1.255(0.4); 0.146(1.9); 0.100(0.5); 0.035(0.4); 0.033(0.4); 0.028(0.7); 0.015(2.2); 0.008(18.9); 0.000(530.8); −0.009
(19.1); −0.017(2.3); −0.019(1.9); −0.022(1.4); −0.023(1.5); −0.024(1.3); −0.027(1.1); −0.046(0.6); −0.076(0.4); −0.150(2.1)
Example 667: ¹H-NMR(399.8 MHz, cdcl3):
δ = 8.523(10.2); 8.520(10.2); 7.984(10.2); 7.980(10.1); 7.650(10.5); 7.645(16.0); 7.623(13.0); 7.599(9.0); 7.594(7.7); 7.578(3.4); 7.573(3.1); 7.519(1.1); 7.409(2.7); 7.390(2.7); 7.309(0.3); 7.292(0.4);
7.288(0.4); 7.282(0.6); 7.260(168.0); 7.237(0.6); 7.235(0.4); 7.234(0.4); 6.996(0.9); 5.013(1.0); 4.993(3.0); 4.975(4.1); 4.956(3.1); 4.937(1.0); 4.374(1.4); 4.356(3.6); 4.336(3.4); 4.318(1.3); 3.628(0.4);
3.610(0.4); 3.518(0.3); 3.503(0.4); 3.141(0.4); 3.123(0.4); 2.714(0.9); 2.711(1.1); 2.695(2.3); 2.674(2.8); 2.669(2.3); 2.665(2.6); 2.662(2.7); 2.658(2.8); 2.646(2.6); 2.643(2.6); 2.627(1.4); 2.624(1.3);
2.492(0.7); 2.475(1.2); 2.461(2.7); 2.445(4.0); 2.437(4.9); 2.429(2.6); 2.419(8.5); 2.401(4.7); 2.398(4.9); 2.388(1.7); 2.380(1.9); 2.377(1.7); 2.367(1.4); 2.349(0.6); 2.215(1.6); 2.198(2.8); 2.189(1.9);
2.183(2.9); 2.173(2.8); 2.170(2.8); 2.167(2.7); 2.158(2.3); 2.152(1.6); 2.143(2.0); 2.126(1.0); 1.545(145.1); 1.517(13.6); 1.515(15.2); 1.512(13.2); 1.324(0.5); 1.306(0.9); 1.288(0.5); 1.282(0.4); 1.255(0.4);
1.156(0.4); 1.139(0.8); 1.121(0.4); 1.018(0.4); 0.882(0.4); 0.864(0.4); 0.169(0.4); 0.146(0.8); 0.049(0.5); 0.008(9.7); 0.000(194.8); −0.009(7.1); −0.016(0.8); −0.018(0.5); −0.150(0.8)
Example 668: ¹H-NMR(399.8 MHz, cdcl3):
δ = 7.460(1.6); 7.439(2.0); 7.352(2.3); 7.346(2.5); 7.272(1.7); 7.266(2.3); 7.260(34.5); 7.251(1.4); 7.246(1.1); 6.387(0.4); 6.371(0.4); 4.754(0.5); 4.732(0.8); 4.710(0.6); 3.731(0.3); 3.707(0.8); 3.683(0.9);
2.725(16.0); 2.450(0.6); 2.429(0.6); 2.424(0.6); 2.418(0.4); 2.406(0.4); 2.394(0.6); 2.370(0.7); 2.045(0.6); 1.998(0.6); 1.994(0.6); 1.971(1.0); 1.948(0.6); 1.944(0.6); 1.755(0.4); 1.749(0.8); 1.729(0.7);
1.723(0.7); 1.703(0.6); 1.544(18.2); 1.259(0.7); 1.255(0.7); 0.008(1.5); 0.000(38.3); −0.008(1.4)
Example 669: ¹H-NMR(399.8 MHz, cdcl3):
δ = 7.419(2.1); 7.416(2.0); 7.331(6.2); 7.327(3.1); 7.260(35.7); 5.759(0.5); 4.987(0.5); 4.969(0.7); 4.952(0.5); 4.239(0.8); 4.221(0.8); 2.676(16.0); 2.617(0.4); 2.604(0.4); 2.600(0.5); 2.587(0.5); 2.583(0.5);
2.571(0.5); 2.422(0.4); 2.406(0.6); 2.397(0.5); 2.388(0.4); 2.381(0.6); 2.365(0.5); 2.345(0.7); 2.341(0.5); 2.328(0.6); 2.324(0.7); 2.320(0.4); 2.315(0.4); 2.294(0.3); 2.069(0.5); 2.054(0.5); 2.044(0.5);
2.039(0.5); 2.030(0.5); 2.014(0.4); 1.542(8.5); 0.008(2.0); 0.000(39.7); −0.008(1.6)
Example 670: ¹H-NMR(399.8 MHz, cdcl3):
δ = 7.547(8.4); 7.526(9.6); 7.407(13.3); 7.394(14.6); 7.385(1.1); 7.355(0.8); 7.341(12.0); 7.335(13.3); 7.271(8.2); 7.260(74.2); 7.250(6.8); 7.245(5.8); 7.184(2.5); 7.163(3.1); 7.159(3.1); 7.139(16.0);
7.127(14.2); 6.996(0.4); 4.902(0.9); 4.880(2.8); 4.858(4.0); 4.836(3.0); 4.814(0.9); 3.790(1.9); 3.767(4.6); 3.743(4.3); 3.719(1.9); 3.533(0.6); 3.518(0.5); 2.758(0.5); 2.495(1.4); 2.473(3.3); 2.452(4.8);
2.449(4.8); 2.431(6.1); 2.405(3.7); 2.382(1.6); 2.091(1.5); 2.068(3.1); 2.065(3.4); 2.042(5.3); 2.019(3.2); 2.015(2.7); 1.991(1.1); 1.753(1.6); 1.746(0.6); 1.731(2.7); 1.725(4.6); 1.715(1.2); 1.705(3.9);
1.699(4.2); 1.678(3.4); 1.672(1.8); 1.652(1.3); 1.559(5.0); 1.277(0.4); 1.259(1.0); 1.242(0.4); 0.146(0.4); 0.008(4.4); 0.000(81.0); −0.008(3.2); −0.149(0.3)
Example 671: ¹H-NMR(399.8 MHz, cdcl3):
δ = 7.519(0.6); 7.446(5.6); 7.425(16.0); 7.420(10.4); 7.336(11.1); 7.331(5.8); 7.324(13.0); 7.310(3.7); 7.305(3.4); 7.278(0.3); 7.277(0.4); 7.276(0.4); 7.275(0.5); 7.273(0.5); 7.272(0.6); 7.271(0.7);
7.261(102.5); 7.250(0.7); 7.249(0.6); 7.248(0.5); 7.245(0.4); 7.244(0.4); 7.242(0.4); 7.027(11.6); 7.014(10.8); 6.996(0.6); 6.673(1.3); 6.656(1.3); 5.125(0.5); 5.107(1.7); 5.089(2.5); 5.071(1.8); 5.053(0.6);
4.282(1.0); 4.264(2.5); 4.245(2.5); 4.226(0.9); 2.653(0.6); 2.649(0.5); 2.638(0.7); 2.634(1.2); 2.632(1.2); 2.628(0.8); 2.623(0.8); 2.619(1.4); 2.616(1.5); 2.613(1.5); 2.609(1.3); 2.604(1.4); 2.602(1.5);
2.598(1.6); 2.594(1.0); 2.590(1.0); 2.586(1.6); 2.583(1.5); 2.580(0.9); 2.568(0.9); 2.564(0.8); 2.478(0.6); 2.461(0.9); 2.447(1.5); 2.432(1.9); 2.423(1.7); 2.416(1.0); 2.407(2.0); 2.388(1.2); 2.384(1.0);
2.370(1.2); 2.367(2.5); 2.363(1.7); 2.358(0.7); 2.354(0.8); 2.350(1.9); 2.346(2.5); 2.342(1.2); 2.337(1.4); 2.333(1.0); 2.329(1.0); 2.325(0.9); 2.319(1.0); 2.316(1.3); 2.298(0.4); 2.295(0.4); 2.145(0.9);
2.131(1.6); 2.121(1.0); 2.116(1.6); 2.106(1.6); 2.104(1.6); 2.101(1.6); 2.091(1.5); 2.086(0.9); 2.084(0.9); 2.077(1.2); 2.060(0.6); 2.045(0.5); 1.543(47.0); 1.259(0.6); 1.255(0.7); 0.147(0.5); 0.033(0.3);
0.017(0.8); 0.014(1.1); 0.013(1.2); 0.008(4.9); 0.000(114.4); −0.008(2.9); −0.149(0.4)
Example 672: ¹H-NMR(399.8 MHz, cdcl3):
δ = 8.081(7.5); 8.079(7.4); 8.061(8.2); 8.058(7.9); 7.689(3.4); 7.686(3.6); 7.670(9.1); 7.667(8.9); 7.651(6.6); 7.648(6.1); 7.601(5.5); 7.598(6.3); 7.581(6.6); 7.578(7.3); 7.561(12.7); 7.540(11.9); 7.518(10.2);
7.515(8.7); 7.500(7.1); 7.496(6.7); 7.356(13.5); 7.351(16.0); 7.312(8.5); 7.307(7.1); 7.291(7.2); 7.286(6.5); 7.260(262.6); 7.177(0.5); 6.996(1.3); 6.855(0.4); 6.833(0.4); 6.060(2.6); 6.039(2.8); 4.892(1.1);
4.870(3.5); 4.848(5.1); 4.826(3.6); 4.803(1.1); 3.708(1.9); 3.684(5.1); 3.661(5.1); 3.637(2.2); 3.532(0.3); 3.511(0.4); 2.528(1.5); 2.506(3.5); 2.485(3.6); 2.481(3.4); 2.461(1.7); 2.436(1.5); 2.412(3.9);
2.388(4.1); 2.365(1.7); 2.171(0.5); 2.158(0.4); 2.045(1.6); 2.021(3.2); 2.018(4.0); 2.007(0.6); 1.995(5.9); 1.972(3.5); 1.968(3.4); 1.945(1.6); 1.758(1.8); 1.738(2.3); 1.732(4.6); 1.712(4.7); 1.706(4.7);
1.685(4.0); 1.679(1.9); 1.659(1.2); 1.538(186.5); 1.509(0.8); 1.285(0.4); 1.255(1.1); 0.146(1.3); 0.101(0.3); 0.008(11.3); 0.000(299.7); −0.008(11.2); −0.149(1.2)
Example 673: ¹H-NMR(399.8 MHz, cdcl3):
δ = 8.671(6.8); 8.667(7.1); 8.659(7.1); 8.655(6.9); 8.546(9.8); 8.541(9.7); 7.858(4.6); 7.853(6.7); 7.848(4.5); 7.838(5.2); 7.833(7.2); 7.828(4.8); 7.519(1.1); 7.447(14.5); 7.442(16.0); 7.421(7.5); 7.400(16.0);
7.387(0.8); 7.369(11.6); 7.364(10.3); 7.348(5.4); 7.343(5.1); 7.332(5.9); 7.320(6.1); 7.313(5.6); 7.300(5.2); 7.260(180.7); 7.215(0.5); 7.193(0.4); 7.158(0.4); 6.996(1.0); 5.685(2.5); 5.667(2.6); 5.156(1.5);
5.136(3.9); 5.118(5.7); 5.099(3.9); 5.079(1.2); 4.299(1.5); 4.282(4.1); 4.262(4.1); 4.244(1.5); 2.661(0.9); 2.656(0.9); 2.642(2.7); 2.637(2.0); 2.627(3.0); 2.622(3.1); 2.617(2.5); 2.612(3.1); 2.607(3.0);
2.597(2.3); 2.592(2.9); 2.587(1.4); 2.578(1.4); 2.573(1.3); 2.409(0.7); 2.393(3.8); 2.389(3.7); 2.378(5.0); 2.369(11.3); 2.354(6.8); 2.349(8.0); 2.340(3.5); 2.330(2.5); 2.318(1.0); 2.298(0.4); 2.171(0.7);
2.118(1.6); 2.100(3.1); 2.089(1.9); 2.075(2.9); 2.072(3.1); 2.066(2.7); 2.056(1.9); 2.049(2.4); 2.046(2.3); 2.027(1.2); 2.006(0.5); 1.553(27.9); 1.333(0.4); 1.294(0.4); 1.284(0.6); 1.254(2.7); 0.880(0.4);
0.146(0.8); 0.070(0.4); 0.008(9.3); 0.000(206.0); −0.008(8.2); −0.025(0.5); −0.029(0.4); −0.150(0.9)
Example 674: ¹H-NMR(399.8 MHz, cdcl3):
δ = 8.468(7.3); 8.464(7.7); 8.457(7.6); 8.452(7.5); 8.121(8.3); 8.116(8.2); 8.102(8.8); 8.097(8.2); 7.528(9.5); 7.520(0.9); 7.507(11.1); 7.382(0.3); 7.357(10.7); 7.354(14.6); 7.348(16.0); 7.345(10.2);
7.338(8.5); 7.326(8.0); 7.305(0.4); 7.284(7.9); 7.279(7.0); 7.260(83.1); 6.996(0.5); 6.813(2.2); 6.793(2.2); 5.299(2.4); 4.876(1.0); 4.855(3.4); 4.833(4.8); 4.811(3.4); 4.789(1.0); 3.773(1.9); 3.749(4.7);
3.726(4.6); 3.702(1.9); 2.506(1.4); 2.483(3.3); 2.462(3.4); 2.458(3.2); 2.438(5.0); 2.414(3.5); 2.391(3.7); 2.389(3.8); 2.366(1.6); 2.067(1.5); 2.056(0.3); 2.044(3.0); 2.041(3.7); 2.018(5.3); 2.014(3.0);
1.995(3.3); 1.991(3.1); 1.969(1.0); 1.788(1.2); 1.768(2.1); 1.762(4.4); 1.742(3.9); 1.736(4.2); 1.716(3.8); 1.710(1.7); 1.689(1.2); 1.550(50.4); 1.284(0.4); 1.254(1.5); 0.146(0.4); 0.008(3.8); 0.000(89.9); −0.009
(3.1); −0.016(0.3); −0.150(0.4)
Example 675: ¹H-NMR(399.8 MHz, cdcl3):
δ = 8.539(13.5); 8.533(16.0); 8.475(16.0); 8.470(13.9); 7.958(2.2); 7.935(2.2); 7.559(0.3); 7.545(9.0); 7.524(10.3); 7.361(0.3); 7.348(0.5); 7.334(11.8); 7.329(13.6); 7.274(8.4); 7.268(9.4); 7.260(179.1);
7.253(9.0); 7.248(6.4); 7.161(0.4); 7.155(0.6); 6.996(1.0); 4.894(1.0); 4.871(3.0); 4.850(4.3); 4.827(3.1); 4.804(1.0); 4.131(0.5); 4.113(0.6); 3.787(1.9); 3.764(4.5); 3.741(4.2); 3.717(1.8); 3.505(0.5);
2.476(1.4); 2.453(3.8); 2.448(3.2); 2.428(6.6); 2.400(3.9); 2.377(1.6); 2.171(0.4); 2.089(1.4); 2.063(3.5); 2.045(4.0); 2.040(5.5); 2.017(3.0); 2.013(2.8); 1.989(1.2); 1.755(1.3); 1.734(2.1); 1.728(4.1);
1.707(3.9); 1.702(4.7); 1.681(3.6); 1.675(1.8); 1.654(1.2); 1.539(97.8); 1.277(0.8); 1.260(1.7); 1.242(0.8); 0.147(0.8); 0.008(9.3); 0.000(202.1); −0.008(7.0); −0.149(0.7)
Example 676: ¹H-NMR(399.8 MHz, cdcl3):
δ = 8.470(0.4); 8.456(14.3); 8.450(15.7); 8.303(16.0); 8.297(14.6); 7.519(1.2); 7.403(6.7); 7.383(11.5); 7.363(11.2); 7.358(14.2); 7.330(8.6); 7.325(6.6); 7.309(4.8); 7.304(4.0); 7.291(0.5); 7.288(0.4);
7.287(0.5); 7.283(0.5); 7.260(207.5); 7.241(1.7); 7.240(1.7); 7.234(2.1); 7.232(2.1); 7.231(2.1); 7.230(2.0); 7.212(1.9); 7.159(0.5); 7.135(0.3); 7.130(0.3); 6.996(1.2); 5.119(0.9); 5.101(2.5); 5.081(3.5);
5.062(2.6); 5.043(0.9); 4.301(1.3); 4.281(3.2); 4.262(3.1); 4.244(1.2); 2.925(0.6); 2.655(0.7); 2.650(0.8); 2.640(0.9); 2.636(1.8); 2.629(1.2); 2.625(1.1); 2.620(2.0); 2.615(2.3); 2.611(2.0); 2.606(2.1);
2.604(1.8); 2.600(2.1); 2.596(1.4); 2.592(1.4); 2.589(1.7); 2.585(2.2); 2.580(1.0); 2.571(1.1); 2.566(1.1); 2.450(0.5); 2.434(1.0); 2.419(2.2); 2.409(1.4); 2.404(2.9); 2.394(3.8); 2.391(3.0); 2.379(3.5);
2.372(4.5); 2.364(2.3); 2.354(3.4); 2.351(4.2); 2.342(1.6); 2.333(1.5); 2.330(1.4); 2.321(1.5); 2.302(0.6); 2.171(0.5); 2.143(1.3); 2.125(2.3); 2.116(1.4); 2.109(1.6); 2.100(2.2); 2.098(2.2); 2.095(2.3);
2.085(1.3); 2.076(1.4); 2.071(1.9); 2.055(0.9); 2.052(0.9); 2.006(0.3); 1.542(124.5); 1.284(0.5); 1.254(2.8); 0.880(0.4); 0.146(0.9); 0.069(1.2); 0.008(8.1); 0.007(5.6); 0.000(233.3); −0.008(8.4); −0.014(1.1);
−0.016(1.0); −0.017(0.9); −0.018(0.8); −0.019(0.7); −0.020(0.6); −0.022(0.6); −0.023(0.5); −0.024(0.5); −0.025(0.5); −0.150(0.9)
Example 677: ¹H-NMR(399.8 MHz, cdcl3):
δ = 7.846(6.0); 7.826(7.6); 7.794(5.5); 7.773(6.5); 7.599(6.5); 7.594(8.6); 7.572(4.7); 7.567(3.3); 7.551(3.6); 7.546(2.8); 7.519(0.6); 7.383(1.1); 7.380(1.0); 7.363(6.2); 7.361(6.7); 7.356(8.9); 7.348(15.9);
7.347(16.0); 7.337(1.5); 7.260(98.6); 7.118(0.4); 7.107(3.5); 7.098(3.3); 7.092(3.3); 7.087(3.8); 7.084(3.5); 7.078(3.0); 7.072(3.1); 7.064(2.9); 6.996(0.6); 5.988(1.9); 5.967(2.0); 5.000(0.7); 4.977(2.5);
4.955(3.5); 4.933(2.6); 4.911(0.8); 3.744(1.0); 3.721(2.7); 3.697(2.8); 3.674(1.0); 2.506(1.1); 2.485(2.7); 2.464(2.8); 2.442(1.3); 2.347(0.9); 2.323(2.6); 2.300(2.8); 2.277(1.2); 2.038(1.2); 2.015(2.2);
2.011(3.0); 1.988(4.2); 1.965(2.7); 1.961(2.4); 1.939(1.3); 1.803(1.2); 1.782(1.4); 1.776(2.9); 1.756(3.0); 1.750(2.8); 1.729(2.5); 1.724(1.2); 1.703(0.8); 1.540(53.3); 1.509(0.8); 0.900(0.3); 0.883(0.8);
0.865(0.4); 0.146(0.5); 0.008(4.7); 0.000(115.3); −0.008(4.0); −0.149(0.5)
Example 678: ¹H-NMR(399.8 MHz, cdcl3):
δ = 7.848(0.4); 7.828(0.5); 7.781(8.1); 7.779(8.4); 7.761(8.8); 7.759(9.0); 7.738(0.4); 7.709(0.4); 7.698(0.4); 7.676(10.5); 7.671(10.8); 7.634(1.3); 7.612(4.5); 7.591(16.0); 7.580(11.6); 7.575(9.8);
7.559(2.6); 7.554(2.6); 7.519(1.5); 7.374(0.5); 7.358(0.6); 7.349(1.3); 7.343(1.2); 7.326(0.4); 7.303(4.5); 7.300(4.7); 7.284(9.9); 7.281(10.2); 7.260(271.1); 7.241(1.0); 7.239(0.8); 7.236(0.7); 7.234(0.6);
7.219(0.4); 7.209(0.4); 7.195(0.4); 7.119(0.3); 7.097(0.4); 7.076(7.7); 7.072(10.1); 7.057(7.1); 7.053(8.8); 7.050(7.4); 7.046(5.0); 7.030(8.1); 7.026(6.8); 7.011(4.9); 7.007(4.1); 6.996(1.7); 6.982(0.4);
6.272(0.5); 5.434(2.5); 5.417(2.6); 5.299(4.1); 5.038(1.0); 5.019(3.1); 5.001(4.2); 4.983(3.0); 4.965(1.0); 4.372(1.3); 4.352(3.5); 4.332(3.6); 4.314(1.3); 2.718(0.9); 2.714(1.0); 2.698(2.3); 2.696(2.2);
2.687(1.5); 2.683(2.3); 2.680(2.6); 2.677(2.9); 2.673(2.1); 2.669(2.6); 2.665(2.8); 2.662(2.8); 2.658(1.7); 2.650(2.8); 2.647(2.9); 2.643(1.5); 2.631(1.5); 2.628(1.4); 2.613(0.4); 2.596(0.7); 2.577(0.6);
2.478(0.7); 2.462(1.2); 2.448(2.5); 2.432(3.5); 2.422(4.9); 2.405(5.5); 2.401(5.5); 2.391(3.0); 2.383(4.2); 2.380(5.1); 2.371(1.9); 2.367(1.5); 2.363(2.1); 2.359(1.9); 2.353(1.4); 2.350(1.6); 2.329(0.7);
2.244(2.0); 2.229(3.4); 2.219(2.2); 2.214(3.4); 2.204(3.0); 2.202(2.9); 2.199(2.9); 2.189(2.5); 2.184(1.6); 2.174(2.1); 2.158(1.1); 2.133(0.4); 2.071(0.4); 2.056(0.4); 2.006(0.4); 1.540(170.2); 1.509(4.3);
1.313(0.5); 1.295(0.9); 1.277(0.5); 1.255(0.7); 1.087(0.3); 1.070(0.8); 1.052(0.4); 0.832(0.4); 0.146(1.2); 0.098(0.3); 0.008(12.9); 0.000(310.0); −0.008(9.8); −0.149(1.2)
Example 679: ¹H-NMR(399.8 MHz, cdcl3):
δ = 7.763(7.3); 7.742(8.9); 7.731(2.2); 7.723(12.6); 7.710(4.3); 7.705(16.0); 7.701(11.6); 7.612(0.4); 7.587(9.2); 7.582(11.5); 7.557(6.0); 7.552(4.5); 7.536(4.7); 7.531(4.0); 7.518(1.0); 7.511(1.7);
7.507(2.8); 7.504(1.9); 7.495(2.1); 7.489(8.2); 7.484(2.6); 7.474(4.3); 7.471(6.9); 7.467(3.6); 7.454(0.4); 7.432(11.6); 7.429(5.4); 7.417(9.0); 7.413(15.8); 7.399(2.7); 7.395(6.0); 7.393(3.8); 7.375(0.3);
7.284(0.3); 7.283(0.3); 7.270(1.0); 7.259(101.8); 6.995(0.6); 6.330(2.5); 6.308(2.5); 5.298(1.0); 4.953(0.9); 4.930(3.2); 4.909(4.7); 4.887(3.2); 4.865(0.9); 3.733(1.3); 3.708(3.6); 3.685(3.6); 3.662(1.3);
2.501(1.5); 2.479(3.6); 2.459(3.8); 2.435(1.8); 2.328(1.4); 2.305(3.5); 2.281(3.7); 2.258(1.6); 1.999(1.7); 1.976(2.9); 1.972(4.0); 1.949(5.5); 1.927(3.6); 1.923(3.3); 1.900(2.0); 1.841(1.7); 1.821(2.0);
1.815(3.9); 1.795(3.9); 1.789(3.7); 1.768(3.2); 1.763(1.5); 1.742(1.0); 1.543(37.5); 1.515(0.6); 1.254(0.8); 0.145(0.5); 0.013(0.9); 0.007(5.0); −0.001(121.9); −0.009(3.8); −0.014(0.5); −0.015(0.3); −0.150(0.5)
Example 680: ¹H-NMR(399.8 MHz, cdcl3):
δ = 7.703(4.8); 7.699(4.5); 7.658(1.0); 7.634(10.3); 7.629(5.4); 7.613(0.6); 7.608(0.6); 7.519(0.6); 7.451(0.8); 7.446(1.4); 7.441(1.0); 7.436(1.0); 7.429(3.3); 7.425(1.5); 7.423(1.5); 7.418(1.1); 7.413(2.0);
7.408(1.7); 7.370(1.3); 7.365(3.1); 7.361(2.0); 7.349(12.4); 7.344(16.0); 7.336(1.3); 7.326(6.1); 7.322(2.3); 7.311(0.8); 7.307(1.5); 7.281(0.4); 7.280(0.4); 7.278(0.4); 7.277(0.5); 7.276(0.5); 7.275(0.6);
7.273(0.7); 7.260(96.0); 7.254(1.6); 7.253(1.1); 7.252(0.8); 7.250(0.5); 7.249(0.4); 6.996(0.5); 5.657(1.0); 5.639(1.0); 5.058(0.5); 5.038(1.6); 5.021(1.9); 5.001(1.6); 4.982(0.5); 4.374(0.6); 4.355(1.5);
4.337(1.5); 4.318(0.5); 2.715(0.4); 2.711(0.4); 2.696(1.1); 2.693(1.1); 2.678(1.4); 2.665(1.3); 2.661(1.3); 2.645(1.3); 2.629(0.5); 2.626(0.5); 2.421(2.6); 2.403(4.8); 2.382(6.1); 2.363(2.6); 2.178(0.7);
2.161(1.0); 2.157(1.2); 2.148(0.8); 2.145(0.8); 2.141(1.1); 2.134(1.2); 2.126(1.2); 2.119(0.6); 2.110(1.1); 2.088(0.5); 1.541(54.1); 1.511(1.1); 0.146(0.5); 0.030(0.3); 0.028(0.3); 0.027(0.4); 0.026(0.4);
0.025(0.4); 0.024(0.4); 0.022(0.5); 0.008(4.8); 0.007(3.9); 0.000(110.0); −0.008(3.3); −0.149(0.4)
Example 684: ¹H-NMR(400.0 MHz, DMSO):
δ = 8.911(12.3); 8.905(15.6); 8.868(16.0); 8.862(12.4); 8.721(5.9); 8.699(6.0); 8.596(0.4); 8.590(0.4); 8.536(0.4); 8.530(0.4); 7.748(7.5); 7.728(10.5); 7.659(9.1); 7.640(15.1); 7.622(9.1); 7.603(4.3);
7.422(5.5); 7.403(8.9); 7.384(3.9); 5.756(0.9); 4.925(0.8); 4.919(0.7); 4.906(2.8); 4.886(4.4); 4.868(3.1); 4.850(1.0); 4.217(1.6); 4.197(4.3); 4.178(4.2); 4.163(1.5); 3.326(87.0); 2.690(1.8); 2.676(0.6);
2.671(0.8); 2.667(0.6); 2.575(1.1); 2.556(3.0); 2.546(1.8); 2.537(4.2); 2.528(4.1); 2.525(4.2); 2.511(46.7); 2.507(85.5); 2.502(109.6); 2.498(80.1); 2.494(42.0); 2.467(4.0); 2.443(3.2); 2.424(1.4); 2.334(0.6);
2.329(0.7); 2.325(0.5); 2.294(0.8); 2.289(1.1); 2.271(2.9); 2.262(2.2); 2.256(3.2); 2.250(4.0); 2.244(3.3); 2.235(2.0); 2.228(2.8); 2.222(2.8); 2.206(1.1); 2.202(1.1); 2.146(1.9); 2.131(2.8); 2.122(2.7);
2.117(2.6); 2.104(3.9); 2.093(3.3); 2.080(2.9); 2.071(0.8); 2.057(0.6); 1.524(0.4); 0.008(1.7); 0.000(42.8); −0.008(1.6)
Example 687: ¹H-NMR(400.0 MHz, DMSO):
δ = 9.278(6.2); 9.256(6.3); 8.996(12.7); 8.991(15.3); 8.947(16.0); 8.941(12.6); 8.316(1.5); 7.870(7.9); 7.850(10.1); 7.725(4.9); 7.706(9.0); 7.686(5.0); 7.678(9.0); 7.658(10.6); 7.460(5.5); 7.441(8.9);
7.422(4.1); 4.901(1.2); 4.879(4.3); 4.857(6.5); 4.835(4.6); 4.813(1.3); 3.817(1.6); 3.795(4.6); 3.771(4.7); 3.748(1.7); 3.326(371.5); 2.676(1.4); 2.671(1.9); 2.667(1.4); 2.507(215.2); 2.502(275.1);
2.498(198.2); 2.334(1.4); 2.329(1.9); 2.325(1.4); 2.291(1.9); 2.269(5.1); 2.247(5.5); 2.224(2.4); 2.207(1.6); 2.184(4.5); 2.161(5.3); 2.138(2.6); 2.115(2.2); 2.091(5.4); 2.067(6.4); 2.043(3.8); 2.019(1.3);
1.770(1.5); 1.745(4.5); 1.719(5.2); 1.698(3.8); 1.673(1.1); 1.236(0.5); 0.146(1.0); 0.008(9.5); 0.000(208.9); −0.008(8.3); −0.150(1.0)
Example 691: ¹H-NMR(600.1 MHz, CD3CN):
δ = 8.519(13.9); 8.517(13.8); 8.511(14.2); 8.509(13.6); 8.075(0.4); 8.073(0.4); 8.062(0.5); 8.059(0.5); 8.051(15.3); 8.049(14.9); 8.037(16.0); 8.035(15.3); 7.967(2.7); 7.965(2.8); 7.954(2.8); 7.952(2.7);
7.883(10.4); 7.870(11.7); 7.676(5.7); 7.663(11.0); 7.655(11.5); 7.643(12.3); 7.402(6.2); 7.390(10.7); 7.377(5.1); 7.364(0.4); 7.356(0.4); 7.350(0.6); 7.344(15.3); 7.336(15.1); 7.330(14.7); 7.323(14.4);
5.447(11.9); 4.906(1.6); 4.891(5.7); 4.876(8.3); 4.861(5.9); 4.846(1.7); 3.878(2.1); 3.862(5.9); 3.846(5.9); 3.831(2.1); 2.349(2.3); 2.348(2.4); 2.333(6.4); 2.320(6.8); 2.317(5.8); 2.306(2.5); 2.305(2.7);
2.303(2.9); 2.268(2.1); 2.253(5.8); 2.252(5.9); 2.237(6.5); 2.221(2.8); 2.139(93.8); 2.133(4.1); 2.117(6.2); 2.116(7.3); 2.100(10.2); 2.085(6.1); 2.083(5.4); 2.067(2.6); 2.055(0.4); 2.051(0.6); 2.047(0.4);
1.964(1.8); 1.956(4.7); 1.952(5.6); 1.948(30.9); 1.944(54.0); 1.940(79.1); 1.936(53.5); 1.932(27.1); 1.923(0.5); 1.912(0.4); 1.829(0.3); 1.825(0.5); 1.821(0.5); 1.817(2.6); 1.802(3.1); 1.799(6.8); 1.785(7.0);
1.782(6.9); 1.768(6.1); 1.764(2.7); 1.750(1.9); 1.341(0.6); 1.284(0.8); 1.269(0.9); 0.005(1.0); 0.000(31.1); −0.006(1.0)
Example 697: ¹H-NMR(399.8 MHz, cdcl3):
δ = 7.766(10.1); 7.745(12.3); 7.672(0.5); 7.632(8.5); 7.629(7.8); 7.628(7.7); 7.614(11.1); 7.609(10.5); 7.596(12.3); 7.591(16.0); 7.569(8.8); 7.563(6.4); 7.548(6.7); 7.542(5.6); 7.519(1.6); 7.393(3.3);
7.389(5.4); 7.373(12.1); 7.369(15.4); 7.363(8.1); 7.350(11.9); 7.345(10.8); 7.330(6.1); 7.325(5.7); 7.321(9.8); 7.316(8.6); 7.302(9.7); 7.298(9.7); 7.284(5.2); 7.280(5.2); 7.260(270.7); 7.236(0.5); 7.234(0.5);
7.231(0.4); 7.228(0.4); 6.996(1.5); 6.459(3.3); 6.439(3.3); 4.980(1.4); 4.958(4.7); 4.936(6.7); 4.914(4.7); 4.892(1.4); 3.737(1.9); 3.714(5.2); 3.691(5.2); 3.666(1.9); 3.522(0.4); 2.510(2.1); 2.488(5.1);
2.467(5.2); 2.445(2.3); 2.403(0.5); 2.386(0.4); 2.345(1.9); 2.321(4.9); 2.299(5.2); 2.275(2.2); 2.099(0.3); 2.056(0.4); 2.022(2.2); 1.998(4.3); 1.995(5.6); 1.972(8.0); 1.949(5.1); 1.945(4.6); 1.922(2.3);
1.817(2.3); 1.791(5.4); 1.770(5.6); 1.764(5.4); 1.744(4.6); 1.738(2.2); 1.717(1.4); 1.545(262.2); 1.517(17.9); 1.515(20.2); 1.512(18.1); 1.258(0.4); 0.899(0.4); 0.883(0.9); 0.864(0.4); 0.146(1.3); 0.100(0.4);
0.035(0.6); 0.033(0.6); 0.030(0.7); 0.008(13.9); 0.000(314.4); −0.008(10.9); −0.016(1.1); −0.018(0.8); −0.019(0.8); −0.022(0.5); −0.023(0.5); −0.024(0.4); −0.025(0.4); −0.027(0.4); −0.150(1.2)
Example 698: ¹H-NMR(399.8 MHz, cdcl3):
δ = 8.455(10.8); 8.451(11.3); 8.444(11.3); 8.440(11.0); 8.164(3.5); 8.142(3.6); 7.812(10.3); 7.809(10.4); 7.792(11.8); 7.789(11.5); 7.775(10.4); 7.754(12.3); 7.739(1.3); 7.634(1.2); 7.629(1.3); 7.596(1.0);
7.590(0.9); 7.575(12.7); 7.569(16.0); 7.547(8.6); 7.542(6.5); 7.526(6.8); 7.521(5.7); 7.371(11.4); 7.360(11.4); 7.350(10.6); 7.339(10.1); 7.260(133.6); 6.996(0.7); 6.272(0.6); 5.177(0.4); 4.967(1.4);
4.945(4.7); 4.922(6.6); 4.900(4.8); 4.877(1.4); 3.795(1.9); 3.772(5.3); 3.749(5.3); 3.726(1.9); 2.614(1.1); 2.596(1.9); 2.577(1.6); 2.467(2.2); 2.445(5.1); 2.423(5.4); 2.400(2.5); 2.351(1.9); 2.327(5.0);
2.303(5.3); 2.280(2.2); 2.167(0.7); 2.067(2.3); 2.040(5.5); 2.017(8.0); 1.994(5.1); 1.990(5.0); 1.967(2.5); 1.950(0.4); 1.805(2.1); 1.779(5.5); 1.758(5.7); 1.753(5.4); 1.732(4.7); 1.706(1.5); 1.551(121.6);
1.521(11.1); 1.255(0.7); 0.882(0.4); 0.863(0.3); 0.146(0.7); 0.007(8.0); −0.001(149.8); −0.009(6.0); −0.150(0.6)
Example 699: ¹H-NMR(399.8 MHz, cdcl3):
δ = 8.268(6.3); 8.265(6.3); 8.257(6.3); 8.254(6.1); 7.742(5.8); 7.738(5.7); 7.721(6.4); 7.718(6.1); 7.655(5.3); 7.633(16.0); 7.586(6.4); 7.580(5.2); 7.565(3.8); 7.559(3.7); 7.546(2.2); 7.528(2.1); 7.520(1.9);
7.291(6.4); 7.280(6.8); 7.271(7.7); 7.260(79.9); 6.996(0.4); 5.008(0.8); 4.989(2.4); 4.971(3.3); 4.953(2.4); 4.934(0.8); 4.361(1.1); 4.343(2.9); 4.324(2.8); 4.305(1.0); 2.699(0.7); 2.696(0.8); 2.679(1.8);
2.661(2.1); 2.659(2.2); 2.654(1.9); 2.649(2.1); 2.646(2.2); 2.643(2.1); 2.631(2.1); 2.628(2.0); 2.612(1.1); 2.483(0.5); 2.466(1.0); 2.452(2.1); 2.436(2.9); 2.426(3.4); 2.422(3.0); 2.411(3.7); 2.404(4.0);
2.395(2.5); 2.383(3.5); 2.373(1.5); 2.364(1.5); 2.352(1.2); 2.332(0.5); 2.211(1.3); 2.194(2.2); 2.185(1.6); 2.179(2.3); 2.167(2.3); 2.154(1.9); 2.148(1.3); 2.139(1.6); 2.123(0.8); 1.597(0.3); 1.549(56.3);
1.519(7.2); 1.499(0.3); 0.146(0.4); −0.001(87.2); −0.008(4.7); −0.150(0.4)
Example 700: ¹H-NMR(399.8 MHz, cdcl3):
δ = 8.702(12.7); 8.699(12.8); 8.068(3.6); 8.049(16.0); 8.046(15.9); 7.748(10.0); 7.727(12.1); 7.673(1.5); 7.614(0.3); 7.592(2.3); 7.583(13.1); 7.577(15.6); 7.556(8.6); 7.551(6.2); 7.535(6.7); 7.530(5.3);
7.519(1.7); 7.360(0.4); 7.260(277.5); 7.240(0.7); 7.234(0.5); 7.211(0.6); 6.996(1.5); 5.839(0.4); 5.823(0.5); 5.802(0.5); 5.299(2.1); 4.930(1.4); 4.907(4.5); 4.885(6.4); 4.863(4.7); 4.840(1.3); 3.807(1.8);
3.783(5.0); 3.760(5.0); 3.736(1.8); 2.755(0.5); 2.737(0.5); 2.722(0.6); 2.712(2.4); 2.701(0.5); 2.693(1.7); 2.687(1.4); 2.675(1.2); 2.486(2.0); 2.463(4.8); 2.442(5.1); 2.419(2.4); 2.359(1.7); 2.336(4.7);
2.313(5.0); 2.289(2.1); 2.081(2.4); 2.071(0.7); 2.054(5.8); 2.031(8.0); 2.008(5.1); 2.005(4.6); 1.982(2.2); 1.848(2.1); 1.827(2.7); 1.821(5.3); 1.801(5.3); 1.795(5.2); 1.775(4.5); 1.769(2.1); 1.748(1.4);
1.646(0.4); 1.597(0.4); 1.545(266.6); 1.517(19.9); 1.514(22.0); 1.512(19.6); 1.294(0.5); 1.284(0.6); 1.255(2.2); 1.034(0.3); 0.880(0.4); 0.846(0.4); 0.831(0.4); 0.821(0.4); 0.146(1.2); 0.008(11.9);
0.000(321.6); −0.009(11.4); −0.050(0.7); −0.150(1.3)
Example 701: ¹H-NMR(399.8 MHz, cdcl3):
δ = 7.744(6.2); 7.723(7.7); 7.596(8.1); 7.591(10.2); 7.568(5.6); 7.562(4.1); 7.547(4.2); 7.541(3.4); 7.518(0.5); 7.386(1.5); 7.371(3.1); 7.365(2.9); 7.355(2.1); 7.350(6.1); 7.344(2.1); 7.334(3.0); 7.328(3.5);
7.313(1.9); 7.297(0.3); 7.259(82.9); 6.995(0.5); 6.953(1.6); 6.949(2.2); 6.943(9.7); 6.923(16.0); 6.902(8.2); 6.896(1.7); 6.185(1.9); 6.166(2.0); 4.963(0.8); 4.941(2.8); 4.919(4.1); 4.897(2.8); 4.875(0.8);
3.721(1.2); 3.698(3.3); 3.674(3.3); 3.651(1.2); 2.510(1.3); 2.488(3.1); 2.467(3.3); 2.445(1.5); 2.337(1.2); 2.314(3.1); 2.291(3.3); 2.267(1.4); 2.007(1.3); 1.984(2.6); 1.980(3.3); 1.957(4.9); 1.934(3.0);
1.930(2.8); 1.908(1.4); 1.821(1.4); 1.794(3.4); 1.774(3.4); 1.768(3.2); 1.748(2.8); 1.721(0.9); 1.536(53.5); 0.146(0.4); 0.008(4.4); 0.000(92.4); −0.009(3.4); −0.150(0.4)
Example 702: ¹H-NMR(399.8 MHz, cdcl3):
δ = −0.019(0.4)
Example 703: ¹H-NMR(399.8 MHz, cdcl3):
δ = 7.747(9.7); 7.726(11.9); 7.685(7.4); 7.667(9.4); 7.603(12.5); 7.598(16.0); 7.573(15.7); 7.569(13.3); 7.553(13.8); 7.539(7.6); 7.520(7.6); 7.502(2.9); 7.492(9.6); 7.472(6.3); 7.359(0.4); 7.259(191.4);
6.995(1.1); 5.983(3.4); 5.963(3.5); 4.971(1.3); 4.948(4.4); 4.926(6.3); 4.904(4.4); 4.882(1.3); 3.696(1.8); 3.672(5.0); 3.649(5.1); 3.625(1.9); 2.503(1.9); 2.479(4.8); 2.458(4.9); 2.436(2.2); 2.332(1.8);
2.309(4.6); 2.285(4.8); 2.262(2.0); 1.981(2.0); 1.958(3.8); 1.954(5.1); 1.931(7.3); 1.908(4.5); 1.904(4.3); 1.882(2.2); 1.802(2.2); 1.781(2.8); 1.775(5.2); 1.755(5.2); 1.749(5.0); 1.729(4.4); 1.702(1.3);
1.533(131.3); 1.293(0.3); 1.255(0.8); 0.146(0.9); 0.008(9.4); 0.000(216.6); −0.009(7.0); −0.150(0.9)
Example 704: ¹H-NMR(399.8 MHz, cdcl3):
δ = 7.674(5.4); 7.609(2.3); 7.590(3.2); 7.586(2.8); 7.568(0.5); 7.549(16.0); 7.546(11.6); 7.518(1.1); 7.515(0.9); 7.499(2.5); 7.496(2.4); 7.482(4.8); 7.477(4.4); 7.462(2.2); 7.460(2.2); 7.443(0.7); 7.259(59.9);
7.229(2.4); 7.225(2.7); 7.209(2.3); 5.366(1.0); 5.349(1.0); 5.018(0.4); 4.998(1.2); 4.981(1.7); 4.962(1.2); 4.943(0.4); 4.358(0.6); 4.340(1.5); 4.319(1.5); 4.302(0.5); 2.706(0.4); 2.703(0.4); 2.687(1.0);
2.676(0.6); 2.672(1.0); 2.669(1.1); 2.667(1.2); 2.663(0.7); 2.661(0.7); 2.657(1.1); 2.654(1.1); 2.651(1.1); 2.647(0.6); 2.639(1.1); 2.636(1.1); 2.632(0.6); 2.620(0.6); 2.617(0.5); 2.438(0.4); 2.423(1.2);
2.407(2.3); 2.399(1.6); 2.390(2.9); 2.383(2.3); 2.368(2.6); 2.359(0.6); 2.350(0.8); 2.348(0.8); 2.338(0.5); 2.187(0.8); 2.171(1.2); 2.162(0.7); 2.155(1.2); 2.146(1.1); 2.143(1.1); 2.140(1.0); 2.131(0.9);
2.123(0.6); 2.116(0.7); 2.099(0.4); 1.535(38.3); 1.415(0.4); 1.293(0.4); 1.255(0.7); 0.016(0.3); 0.008(2.5); 0.000(65.3); −0.009(2.2)
Example 705: ¹H-NMR(399.8 MHz, cdcl3):
δ = 7.743(0.5); 7.722(0.6); 7.705(0.4); 7.677(11.1); 7.673(11.3); 7.624(4.8); 7.602(16.0); 7.589(11.8); 7.584(9.6); 7.568(3.4); 7.563(3.1); 7.537(13.8); 7.516(15.8); 7.502(0.5); 7.421(0.4); 7.405(0.6);
7.400(0.6); 7.328(0.3); 7.289(13.5); 7.284(16.1); 7.260(89.2); 7.252(12.0); 7.247(9.0); 7.231(8.7); 7.226(7.3); 6.996(0.5); 6.033(2.8); 6.015(2.9); 5.054(1.3); 5.034(3.8); 5.017(5.1); 4.998(3.9); 4.979(1.2);
4.369(1.5); 4.351(3.9); 4.331(3.9); 4.312(1.4); 2.714(0.9); 2.711(1.0); 2.695(3.0); 2.692(2.3); 2.679(3.0); 2.675(3.4); 2.671(2.1); 2.664(3.4); 2.660(3.1); 2.645(3.1); 2.628(1.4); 2.625(1.3); 2.457(0.4);
2.440(0.6); 2.425(5.0); 2.409(6.0); 2.403(12.3); 2.394(3.8); 2.385(12.3); 2.369(3.7); 2.365(3.2); 2.353(0.8); 2.332(0.4); 2.196(1.8); 2.178(2.8); 2.172(2.2); 2.163(2.6); 2.151(3.0); 2.143(2.6); 2.138(1.8);
2.128(2.1); 2.107(1.1); 1.971(0.3); 1.553(53.7); 1.523(4.9); 1.415(0.6); 1.293(0.8); 1.284(0.6); 1.277(0.6); 1.255(2.5); 1.064(0.3); 0.881(0.5); 0.864(0.4); 0.846(0.5); 0.146(0.5); 0.069(17.8); 0.007(5.3); −0.001
(99.3); −0.009(5.4); −0.150(0.5)
Example 706: ¹H-NMR(399.8 MHz, cdcl3):
δ = 7.741(5.4); 7.720(6.5); 7.696(0.4); 7.662(13.5); 7.658(5.0); 7.646(5.7); 7.641(15.5); 7.635(2.4); 7.629(1.0); 7.590(6.9); 7.585(8.5); 7.560(4.6); 7.555(3.5); 7.539(3.6); 7.534(2.9); 7.519(0.7); 7.406(2.8);
7.400(16.0); 7.395(5.3); 7.383(5.1); 7.379(13.1); 7.373(1.9); 7.297(1.0); 7.292(1.1); 7.260(108.1); 6.996(0.5); 6.282(2.1); 6.262(2.1); 4.914(0.7); 4.891(2.5); 4.869(3.5); 4.847(2.4); 4.825(0.7); 3.729(1.0);
3.706(2.8); 3.682(2.8); 3.659(1.0); 2.499(1.1); 2.477(2.7); 2.456(2.9); 2.432(1.4); 2.326(1.1); 2.303(2.7); 2.280(2.8); 2.256(1.2); 1.999(1.1); 1.973(2.9); 1.950(4.0); 1.927(2.6); 1.924(2.4); 1.901(1.4);
1.855(1.3); 1.829(2.8); 1.808(2.9); 1.803(2.7); 1.782(2.3); 1.755(0.7); 1.549(123.9); 1.521(7.4); 1.519(8.3); 1.516(7.3); 0.146(0.5); 0.008(5.7); 0.000(122.2); −0.009(5.5); −0.150(0.5)
Example 707: ¹H-NMR(399.8 MHz, cdcl3):
δ = 7.698(5.4); 7.648(0.5); 7.629(16.0); 7.626(11.3); 7.604(0.3); 7.519(0.5); 7.320(1.7); 7.312(1.3); 7.304(2.3); 7.297(20.4); 7.292(20.7); 7.285(2.1); 7.277(1.7); 7.271(2.6); 7.269(3.3); 7.260(94.8);
6.996(0.5); 5.592(1.1); 5.572(1.2); 5.039(0.5); 5.019(1.5); 5.001(1.9); 4.982(1.5); 4.963(0.5); 4.367(0.5); 4.349(1.5); 4.331(1.5); 4.312(0.5); 2.710(0.3); 2.707(0.4); 2.689(1.1); 2.674(1.3); 2.671(1.2);
2.662(1.3); 2.658(1.3); 2.642(1.3); 2.625(0.6); 2.622(0.5); 2.420(2.4); 2.402(4.9); 2.381(5.7); 2.362(2.5); 2.175(0.7); 2.155(1.0); 2.152(1.3); 2.143(0.8); 2.136(1.2); 2.129(1.2); 2.123(1.1); 2.114(0.6);
2.105(1.1); 2.085(0.4); 1.548(102.4); 1.519(6.1); 1.517(6.9); 1.514(6.1); 0.146(0.4); 0.008(5.0); 0.000(108.0); −0.009(4.0); −0.150(0.4)
Example 708: ¹H-NMR(399.8 MHz, cdcl3):
δ = 7.740(9.0); 7.719(11.5); 7.710(9.3); 7.705(14.8); 7.701(9.0); 7.593(12.1); 7.587(16.0); 7.582(9.5); 7.578(6.5); 7.562(15.9); 7.540(5.9); 7.535(4.8); 7.519(0.8); 7.475(4.8); 7.472(5.5); 7.470(5.1);
7.467(4.6); 7.455(7.2); 7.452(7.4); 7.450(7.6); 7.447(6.1); 7.372(10.1); 7.353(14.7); 7.333(6.1); 7.260(118.9); 6.996(0.6); 6.298(3.5); 6.278(3.5); 4.924(1.3); 4.902(4.1); 4.880(5.9); 4.858(4.1); 4.836(1.2);
3.737(1.7); 3.713(4.7); 3.690(4.7); 3.666(1.7); 2.497(1.9); 2.475(4.7); 2.454(4.8); 2.431(2.2); 2.333(1.8); 2.310(4.5); 2.286(4.8); 2.263(1.9); 2.008(2.0); 1.982(4.9); 1.959(6.9); 1.936(4.4); 1.932(4.1);
1.910(2.2); 1.850(2.1); 1.824(4.7); 1.804(5.0); 1.798(4.6); 1.777(3.9); 1.751(1.2); 1.603(0.3); 1.551(137.4); 1.523(9.1); 1.521(10.0); 1.518(8.6); 0.146(0.6); 0.008(7.4); 0.000(136.2); −0.009(5.9); −0.150(0.6)
Example 709: ¹H-NMR(399.8 MHz, cdcl3):
δ = 7.709(5.6); 7.660(0.4); 7.639(13.9); 7.635(16.0); 7.616(0.3); 7.519(0.5); 7.412(1.9); 7.409(2.1); 7.406(2.3); 7.404(2.1); 7.392(2.5); 7.389(2.8); 7.387(3.0); 7.384(2.8); 7.318(3.3); 7.314(5.4); 7.309(3.1);
7.281(3.0); 7.260(70.8); 7.241(3.5); 7.196(3.1); 7.192(4.2); 7.189(2.9); 7.176(2.0); 7.173(2.6); 7.169(1.7); 6.996(0.4); 5.600(1.0); 5.581(1.1); 5.044(0.5); 5.024(1.6); 5.007(1.9); 4.988(1.6); 4.968(0.5);
4.377(0.5); 4.358(1.5); 4.340(1.5); 4.322(0.5); 2.714(0.4); 2.710(0.4); 2.695(1.0); 2.692(1.1); 2.683(0.6); 2.677(1.3); 2.664(1.3); 2.660(1.3); 2.655(0.7); 2.645(1.4); 2.628(0.6); 2.625(0.5); 2.426(2.7);
2.408(4.7); 2.386(6.2); 2.368(2.6); 2.174(0.8); 2.155(1.0); 2.151(1.2); 2.142(0.8); 2.139(0.8); 2.135(1.1); 2.128(1.1); 2.120(1.2); 2.113(0.6); 2.104(1.1); 2.100(0.6); 2.082(0.5); 1.550(48.6); 1.519(4.0);
0.008(2.6); 0.000(74.3); −0.009(2.6)
Example 710: ¹H-NMR(399.8 MHz, cdcl3):
δ = 7.744(5.8); 7.723(7.0); 7.591(7.7); 7.585(9.4); 7.555(5.1); 7.550(4.1); 7.534(4.1); 7.529(3.4); 7.519(0.5); 7.442(0.7); 7.429(0.8); 7.394(11.8); 7.388(1.3); 7.381(13.1); 7.260(63.4); 7.229(0.8); 7.216(0.7);
7.130(16.0); 7.117(13.9); 6.996(0.4); 4.988(0.8); 4.966(2.6); 4.944(3.9); 4.922(2.7); 4.900(0.8); 4.396(0.6); 3.794(1.2); 3.770(3.2); 3.746(3.2); 3.723(1.2); 2.510(1.2); 2.488(3.0); 2.466(3.1); 2.443(1.4);
2.368(1.1); 2.345(2.9); 2.321(3.1); 2.297(1.3); 2.069(1.3); 2.046(2.6); 2.043(3.3); 2.019(4.8); 1.997(2.9); 1.993(2.8); 1.970(1.6); 1.964(0.8); 1.808(1.3); 1.782(3.2); 1.761(3.3); 1.756(3.3); 1.735(3.0);
1.729(1.5); 1.708(0.9); 1.547(54.4); 0.008(3.1); 0.000(69.4); −0.008(2.8)
Example 711: ¹H-NMR(399.8 MHz, cdcl3):
δ = 7.711(6.0); 7.689(16.0); 7.610(5.5); 7.605(5.1); 7.589(3.5); 7.584(3.4); 7.329(9.9); 7.316(10.8); 7.261(43.6); 7.016(10.9); 7.003(10.1); 6.626(2.2); 6.611(2.3); 5.300(2.5); 5.072(0.9); 5.053(2.9);
5.035(4.1); 5.016(2.9); 4.997(0.9); 4.368(1.2); 4.349(3.0); 4.330(2.9); 4.312(1.1); 2.709(0.8); 2.693(2.0); 2.674(2.4); 2.668(1.9); 2.658(2.4); 2.643(2.2); 2.624(1.1); 2.464(0.7); 2.448(2.4); 2.439(3.1);
2.433(3.6); 2.419(7.1); 2.408(4.7); 2.399(5.5); 2.379(1.9); 2.368(0.9); 2.348(0.4); 2.239(1.4); 2.222(2.3); 2.207(1.9); 2.195(2.5); 2.193(2.4); 2.173(1.4); 2.168(1.6); 2.149(0.8); 1.552(48.1); 1.255(0.5);
0.000(46.5)
Example 712: ¹H-NMR(399.8 MHz, cdcl3):
δ = 8.130(0.3); 8.064(8.6); 8.062(8.6); 8.044(9.5); 8.042(9.2); 7.756(8.6); 7.736(10.8); 7.672(4.0); 7.669(4.2); 7.653(10.2); 7.650(10.1); 7.635(7.4); 7.632(7.0); 7.607(10.9); 7.602(16.0); 7.593(10.8);
7.590(11.7); 7.586(10.7); 7.570(12.6); 7.567(12.5); 7.550(4.3); 7.547(4.1); 7.528(0.5); 7.519(0.6); 7.464(10.0); 7.461(9.6); 7.445(8.4); 7.442(7.8); 7.410(0.4); 7.260(68.3); 6.996(0.4); 6.030(3.6); 6.009(3.6);
4.949(1.2); 4.926(4.2); 4.904(6.0); 4.883(4.2); 4.860(1.2); 3.721(1.8); 3.697(4.8); 3.674(5.0); 3.651(1.9); 2.537(1.8); 2.515(4.4); 2.494(4.6); 2.472(2.2); 2.343(1.7); 2.319(4.4); 2.296(4.7); 2.272(2.0);
2.022(1.8); 1.998(3.7); 1.995(4.7); 1.972(6.8); 1.949(4.1); 1.945(4.0); 1.922(1.9); 1.837(1.9); 1.811(4.6); 1.790(4.8); 1.785(4.6); 1.764(3.9); 1.738(1.1); 1.549(38.3); 1.293(0.4); 1.255(0.9); 0.882(0.5);
0.008(4.1); 0.000(75.3); −0.008(3.6); −0.150(0.3)
Example 713: ¹H-NMR(399.8 MHz, cdcl3):
δ = 7.973(3.1); 7.970(3.0); 7.953(3.5); 7.950(3.3); 7.719(0.4); 7.692(5.6); 7.623(0.5); 7.587(1.8); 7.584(2.1); 7.571(16.0); 7.568(15.0); 7.550(3.1); 7.546(2.8); 7.528(2.5); 7.524(2.7); 7.508(3.0); 7.505(3.0);
7.489(1.4); 7.485(1.2); 7.260(28.4); 7.129(3.4); 7.125(3.4); 7.111(3.1); 7.107(3.0); 5.344(1.2); 5.327(1.3); 5.027(0.5); 5.007(1.5); 4.990(2.0); 4.972(1.5); 4.952(0.5); 4.366(0.6); 4.348(1.7); 4.328(1.6);
4.309(0.6); 2.735(0.4); 2.732(0.4); 2.716(1.2); 2.696(1.4); 2.685(1.3); 2.680(1.2); 2.665(1.2); 2.649(0.6); 2.646(0.5); 2.431(1.6); 2.415(2.2); 2.408(4.8); 2.399(1.6); 2.390(4.6); 2.374(1.7); 2.370(1.3);
2.357(0.3); 2.272(0.9); 2.256(1.3); 2.247(0.9); 2.240(1.4); 2.228(1.4); 2.221(1.0); 2.206(0.9); 2.202(0.9); 2.184(0.5); 2.169(0.5); 1.551(19.9); 1.254(0.9); 0.007(1.7); −0.001(29.9); −0.009(1.4)
Example 714: ¹H-NMR(399.8 MHz, cdcl3):
δ = 8.661(0.8); 8.490(0.8); 7.838(2.5); 7.818(2.7); 7.703(6.3); 7.655(0.8); 7.635(16.0); 7.519(0.4); 7.312(1.4); 7.260(44.2); 5.677(1.4); 5.661(1.5); 5.065(0.6); 5.046(1.7); 5.028(2.4); 5.009(1.8); 4.990(0.5);
4.384(0.7); 4.365(1.9); 4.346(1.9); 4.327(0.7); 2.724(0.5); 2.708(1.3); 2.689(1.6); 2.677(1.6); 2.673(1.5); 2.658(1.4); 2.641(0.7); 2.439(2.6); 2.418(5.2); 2.399(6.1); 2.381(2.2); 2.198(0.8); 2.180(1.3);
2.165(1.2); 2.153(1.4); 2.145(1.4); 2.129(1.2); 2.108(0.5); 1.566(6.8); 1.253(0.4); −0.001(48.1)
Example 715: ¹H-NMR(399.8 MHz, cdcl3):
δ = 8.459(9.4); 8.454(10.0); 8.447(9.9); 8.442(9.5); 8.074(10.3); 8.069(10.2); 8.055(11.0); 8.050(10.3); 7.741(9.8); 7.720(12.3); 7.602(12.9); 7.597(16.0); 7.574(9.0); 7.569(6.8); 7.553(6.8); 7.548(5.6);
7.521(0.5); 7.359(0.5); 7.341(10.8); 7.329(10.8); 7.321(10.4); 7.310(10.0); 7.261(69.4); 6.997(0.4); 6.756(3.7); 6.736(3.7); 5.300(2.6); 4.949(1.4); 4.927(4.7); 4.906(6.8); 4.884(4.7); 4.862(1.4); 3.769(2.0);
3.745(5.4); 3.722(5.4); 3.699(1.9); 2.521(2.1); 2.499(5.0); 2.477(5.3); 2.455(2.4); 2.362(2.0); 2.339(5.1); 2.315(5.4); 2.291(2.2); 2.054(2.1); 2.030(4.4); 2.027(5.2); 2.004(7.7); 1.981(4.7); 1.977(4.5);
1.954(2.1); 1.853(2.1); 1.827(5.2); 1.806(5.5); 1.800(5.2); 1.780(4.4); 1.753(1.4); 1.562(39.3); 1.255(1.3); 0.146(0.4); 0.000(73.2); −0.008(3.2); −0.149(0.3)
Example 716: ¹H-NMR(399.8 MHz, cdcl3):
δ = 8.526(14.8); 8.471(15.2); 8.415(0.5); 8.009(0.4); 7.927(5.3); 7.907(5.3); 7.751(9.4); 7.730(10.8); 7.578(16.0); 7.560(10.3); 7.538(7.8); 7.261(41.1); 4.947(1.7); 4.925(5.0); 4.903(7.0); 4.881(4.9);
4.858(1.6); 3.793(2.7); 3.770(6.3); 3.747(6.2); 3.723(2.4); 3.609(0.3); 3.521(0.4); 2.611(0.4); 2.597(0.5); 2.576(0.4); 2.485(3.0); 2.465(6.3); 2.442(6.5); 2.420(3.1); 2.354(2.8); 2.332(6.2); 2.308(6.3);
2.286(2.8); 2.164(0.4); 2.114(0.4); 2.067(2.2); 2.042(5.3); 2.018(7.3); 1.993(5.0); 1.969(1.8); 1.896(0.4); 1.843(2.3); 1.817(5.5); 1.794(6.3); 1.769(4.4); 1.744(1.5); 1.551(57.3); 1.306(0.5); 1.287(0.5);
1.258(0.7); 1.140(0.3); 0.863(0.4); 0.846(0.4); 0.144(0.4); 0.114(0.4); −0.001(44.8)
Example 717: ¹H-NMR(399.8 MHz, cdcl3):
δ = 8.531(0.4); 8.471(0.5); 8.466(0.7); 8.453(14.7); 8.447(15.4); 8.433(0.4); 8.296(16.0); 8.291(14.9); 7.648(10.2); 7.642(15.0); 7.620(12.4); 7.596(8.9); 7.591(7.5); 7.575(3.4); 7.570(3.0); 7.520(0.4);
7.294(0.5); 7.261(59.3); 7.232(2.4); 6.997(0.3); 5.023(0.9); 5.003(3.0); 4.985(4.0); 4.967(3.1); 4.947(1.0); 4.376(1.3); 4.357(3.3); 4.338(3.3); 4.319(1.2); 2.804(1.1); 2.716(0.8); 2.713(0.9); 2.697(2.2);
2.682(2.3); 2.676(2.6); 2.672(2.1); 2.667(2.4); 2.664(2.4); 2.661(2.6); 2.649(2.4); 2.646(2.5); 2.642(1.4); 2.630(1.3); 2.627(1.2); 2.487(0.5); 2.471(0.9); 2.456(2.6); 2.440(5.3); 2.432(3.6); 2.420(6.3);
2.417(6.9); 2.399(6.6); 2.389(1.5); 2.381(1.9); 2.368(1.2); 2.350(0.5); 2.214(1.6); 2.197(2.6); 2.189(1.7); 2.182(2.6); 2.172(2.5); 2.170(2.6); 2.167(2.5); 2.158(2.1); 2.149(1.5); 2.142(1.9); 2.125(1.0);
1.555(20.6); 1.255(0.8); 0.008(3.1); 0.000(61.7); −0.008(2.5)
Example 718: ¹H-NMR(399.8 MHz, cdcl3):
δ = 7.855(5.5); 7.730(2.2); 7.709(2.7); 7.575(2.9); 7.570(3.5); 7.540(1.9); 7.534(1.6); 7.519(1.8); 7.513(1.3); 7.260(49.5); 6.930(1.8); 6.795(3.7); 6.659(1.9); 6.554(0.7); 4.875(0.9); 4.854(1.3); 4.832(0.9);
3.974(0.5); 3.918(0.4); 3.897(16.0); 3.820(0.5); 3.687(0.4); 3.664(1.2); 3.641(1.2); 3.617(0.4); 2.459(0.5); 2.437(1.1); 2.416(1.2); 2.392(0.6); 2.319(0.4); 2.296(1.1); 2.272(1.2); 2.249(0.5); 1.974(0.4);
1.947(1.1); 1.924(1.7); 1.901(1.0); 1.897(1.0); 1.875(0.5); 1.787(0.5); 1.761(1.2); 1.741(1.2); 1.735(1.2); 1.715(1.0); 1.688(0.4); 1.547(49.6); 0.008(2.0); 0.000(55.4); −0.008(2.3)
Example 719: ¹H-NMR(399.8 MHz, cdcl3):
δ = 7.798(5.8); 7.637(3.2); 7.633(3.3); 7.602(1.2); 7.581(4.5); 7.571(3.4); 7.566(2.8); 7.549(0.8); 7.544(0.8); 7.260(25.3); 6.634(1.7); 6.499(3.4); 6.363(1.7); 6.090(0.7); 5.299(1.6); 5.000(0.9); 4.982(1.3);
4.963(0.9); 4.309(0.4); 4.290(1.0); 4.271(1.0); 4.252(0.4); 3.842(16.0); 2.652(0.8); 2.648(0.6); 2.641(0.4); 2.636(0.8); 2.632(0.9); 2.627(0.6); 2.621(0.8); 2.616(0.9); 2.613(0.5); 2.601(0.8); 2.597(0.4);
2.586(0.4); 2.582(0.4); 2.390(1.1); 2.386(1.0); 2.374(1.4); 2.366(3.2); 2.359(1.0); 2.347(2.5); 2.334(1.0); 2.327(0.7); 2.147(0.5); 2.130(0.8); 2.123(0.5); 2.114(0.6); 2.105(0.8); 2.102(0.8); 2.095(0.7);
2.089(0.5); 2.083(0.5); 2.076(0.6); 2.057(0.3); 1.561(10.2); 1.433(0.6); 1.254(0.5); 0.008(1.2); −0.001(27.4); −0.009(1.1)
Example 720: ¹H-NMR(399.8 MHz, cdcl3):
δ = 8.762(9.6); 8.751(9.1); 7.865(8.9); 7.845(9.7); 7.727(10.0); 7.706(12.9); 7.646(0.8); 7.608(16.0); 7.585(9.8); 7.556(11.2); 7.543(8.7); 7.535(8.0); 7.523(7.6); 7.462(0.8); 7.260(492.6); 6.996(3.0);
6.011(4.8); 5.990(5.0); 4.945(1.6); 4.922(5.1); 4.900(7.3); 4.878(5.1); 4.855(1.5); 3.720(2.3); 3.695(6.2); 3.672(5.9); 3.649(2.7); 3.609(1.0); 3.535(1.2); 3.517(1.4); 3.510(1.3); 2.520(2.4); 2.494(5.7);
2.472(6.2); 2.452(2.8); 2.344(2.5); 2.322(5.9); 2.297(6.5); 2.275(2.8); 2.070(0.8); 2.055(1.0); 2.007(2.3); 1.981(5.2); 1.957(7.6); 1.931(5.3); 1.907(2.2); 1.839(2.4); 1.813(5.7); 1.792(5.9); 1.766(4.4);
1.741(1.8); 1.690(0.8); 1.655(1.1); 1.643(1.5); 1.543(709.6); 1.494(3.6); 1.457(1.3); 1.444(1.4); 1.432(1.0); 1.413(1.2); 1.375(0.9); 1.339(0.9); 1.295(2.2); 1.256(8.3); 1.220(1.2); 1.215(1.2); 1.053(0.7);
0.890(0.9); 0.881(1.3); 0.844(2.4); 0.146(2.7); 0.102(1.0); 0.086(1.2); 0.000(530.6); −0.072(1.2); −0.084(1.1); −0.095(0.9); −0.150(2.5)
Example 721: ¹H-NMR(399.8 MHz, cdcl3):
δ = 8.696(3.2); 8.687(3.2); 7.676(6.4); 7.640(2.8); 7.638(2.9); 7.621(3.6); 7.619(3.6); 7.581(0.6); 7.559(14.1); 7.557(16.0); 7.537(0.4); 7.521(0.4); 7.490(3.2); 7.479(3.2); 7.471(2.6); 7.459(2.4); 7.262(23.2);
5.396(1.5); 5.379(1.5); 5.008(0.5); 4.989(1.5); 4.972(2.1); 4.954(1.5); 4.935(0.5); 4.368(0.7); 4.349(1.9); 4.329(1.9); 4.311(0.7); 2.718(0.5); 2.715(0.5); 2.698(1.2); 2.678(1.4); 2.675(1.1); 2.669(1.3);
2.666(1.4); 2.663(1.3); 2.650(1.3); 2.647(1.3); 2.632(0.7); 2.629(0.6); 2.474(0.3); 2.458(0.6); 2.443(1.4); 2.427(2.7); 2.419(1.9); 2.403(3.9); 2.386(3.2); 2.376(0.8); 2.367(1.0); 2.355(0.6); 2.200(0.9);
2.184(1.4); 2.174(1.0); 2.168(1.5); 2.156(1.4); 2.144(1.2); 2.136(0.8); 2.129(0.9); 2.112(0.5); 1.564(34.1); 1.255(0.4); 0.008(1.7); 0.000(25.6); −0.008(1.3)
Example 722: ¹H-NMR(399.8 MHz, cdcl3):
δ = 7.772(9.6); 7.751(11.6); 7.598(12.1); 7.592(15.3); 7.570(16.0); 7.564(7.1); 7.553(12.2); 7.551(14.2); 7.543(5.9); 7.519(0.7); 7.499(8.2); 7.495(8.6); 7.480(10.3); 7.476(10.5); 7.355(5.3); 7.353(5.5);
7.337(11.3); 7.334(11.1); 7.318(6.8); 7.315(6.4); 7.278(7.6); 7.274(8.0); 7.260(73.6); 7.240(4.2); 7.236(3.8); 6.996(0.4); 6.234(3.4); 6.212(3.5); 4.988(1.3); 4.965(4.5); 4.943(6.5); 4.921(4.5); 4.899(1.3);
3.738(1.8); 3.714(5.1); 3.691(5.1); 3.667(1.8); 2.504(2.0); 2.482(4.8); 2.461(5.0); 2.439(2.3); 2.345(1.8); 2.322(4.7); 2.298(5.1); 2.274(2.1); 2.027(2.0); 2.003(4.0); 2.000(5.0); 1.977(7.3); 1.954(4.6);
1.950(4.3); 1.927(2.1); 1.805(2.0); 1.784(2.7); 1.779(5.1); 1.758(5.2); 1.753(5.0); 1.732(4.3); 1.706(1.3); 1.554(46.1); 1.294(0.5); 1.277(0.4); 1.255(1.0); 0.900(0.6); 0.882(1.4); 0.864(0.7); 0.146(0.3);
0.008(3.7); 0.000(72.9); −0.008(3.0)
Example 723: ¹H-NMR(399.8 MHz, cdcl3):
δ = 7.706(0.6); 7.673(12.6); 7.669(13.0); 7.620(5.8); 7.599(16.0); 7.579(11.1); 7.574(9.6); 7.558(3.6); 7.553(3.4); 7.519(1.2); 7.483(9.1); 7.481(8.9); 7.463(10.5); 7.461(10.3); 7.359(6.7); 7.354(7.2);
7.340(11.2); 7.335(11.5); 7.311(0.6); 7.296(6.2); 7.293(6.5); 7.287(1.5); 7.286(1.6); 7.278(12.1); 7.275(12.2); 7.260(217.0); 7.250(2.5); 7.236(0.7); 7.231(0.7); 7.222(7.4); 7.218(7.2); 7.203(8.2); 7.198(7.5);
7.184(4.1); 7.180(3.6); 6.996(1.3); 5.780(2.8); 5.764(2.9); 5.300(1.5); 5.060(1.2); 5.040(3.8); 5.023(5.1); 5.004(3.8); 4.985(1.2); 4.372(1.6); 4.352(4.3); 4.333(4.0); 4.314(1.5); 2.716(1.1); 2.713(1.1);
2.696(2.8); 2.681(2.8); 2.676(3.3); 2.672(2.4); 2.667(3.0); 2.664(3.0); 2.661(3.2); 2.649(3.0); 2.646(3.1); 2.630(1.6); 2.627(1.5); 2.465(0.6); 2.449(1.0); 2.433(3.2); 2.419(6.1); 2.409(4.7); 2.401(8.7);
2.393(6.4); 2.380(7.4); 2.370(1.8); 2.362(2.4); 2.349(1.3); 2.329(0.6); 2.218(2.3); 2.201(3.5); 2.191(2.3); 2.185(3.4); 2.176(3.2); 2.173(3.3); 2.170(3.2); 2.161(2.5); 2.152(1.8); 2.146(2.3); 2.128(1.1);
1.544(170.0); 1.255(1.1); 0.836(0.4); 0.146(0.9); 0.051(0.4); 0.025(0.7); 0.024(0.7); 0.023(0.7); 0.022(0.9); 0.020(1.0); 0.019(1.1); 0.017(1.4); 0.008(10.5); 0.000(239.2); −0.008(9.0); −0.033(0.4); −0.040
(0.3); −0.050(0.5); −0.149(0.9)
Example 724: ¹H-NMR(399.8 MHz, cdcl3):
δ = 8.677(4.7); 8.672(4.8); 8.663(3.6); 8.660(3.5); 8.651(3.3); 8.648(3.2); 7.929(2.1); 7.924(3.1); 7.920(2.1); 7.910(2.3); 7.905(3.3); 7.900(2.2); 7.519(0.3); 7.321(13.2); 7.313(3.7); 7.301(16.0); 7.294(3.4);
7.260(58.8); 7.103(4.4); 7.083(5.6); 7.063(3.1); 6.560(1.2); 6.541(1.2); 5.250(0.5); 5.229(1.3); 5.216(1.4); 5.208(1.4); 5.195(1.4); 5.174(0.5); 4.879(1.1); 4.857(2.7); 4.834(2.7); 4.812(1.0); 3.231(0.6);
3.211(1.0); 3.202(0.9); 3.184(1.6); 3.181(1.6); 3.174(0.9); 3.163(1.0); 3.153(1.1); 3.135(0.7); 2.803(1.0); 2.733(0.6); 2.712(1.4); 2.702(0.9); 2.695(1.4); 2.681(1.8); 2.673(0.9); 2.663(1.6); 2.642(0.8);
2.512(0.6); 2.509(0.7); 2.498(0.7); 2.494(0.8); 2.486(1.0); 2.473(1.2); 2.467(1.1); 2.463(1.1); 2.452(1.0); 2.442(1.1); 2.431(0.4); 2.421(0.7); 2.417(0.6); 2.394(1.0); 2.380(1.4); 2.365(1.6); 2.352(1.7);
2.337(1.3); 2.334(1.1); 2.322(1.0); 2.309(0.5); 2.044(0.5); 1.774(1.0); 1.569(12.6); 1.284(0.3); 1.276(0.4); 1.254(2.9); 0.880(0.4); 0.007(3.1); −0.001(58.5); −0.009(1.9)
Example 725: ¹H-NMR(399.8 MHz, cdcl3):
δ = 8.406(2.5); 8.401(2.6); 8.394(2.6); 8.389(2.6); 7.925(2.8); 7.920(2.8); 7.906(3.1); 7.901(2.9); 7.313(7.8); 7.305(0.6); 7.293(10.2); 7.284(3.3); 7.272(3.4); 7.262(38.9); 7.253(3.2); 7.125(2.9); 7.105(3.3);
7.085(2.0); 6.788(0.7); 6.774(0.7); 5.255(0.6); 5.253(0.7); 5.250(0.7); 5.248(0.7); 5.241(0.8); 5.238(0.8); 5.236(0.9); 5.234(0.9); 5.231(0.8); 5.228(0.8); 5.221(0.7); 5.219(0.8); 5.214(0.7); 4.758(0.7);
4.736(1.9); 4.713(1.9); 4.691(0.7); 3.229(0.4); 3.227(0.4); 3.209(0.6); 3.204(0.6); 3.201(0.6); 3.197(0.5); 3.186(0.7); 3.182(1.1); 3.179(1.0); 3.175(0.7); 3.171(0.5); 3.163(0.5); 3.160(0.6); 3.157(0.7);
3.152(0.8); 3.133(0.4); 3.131(0.4); 2.662(0.4); 2.641(1.0); 2.630(0.6); 2.621(1.1); 2.610(1.1); 2.602(0.6); 2.590(1.2); 2.572(0.6); 2.535(0.4); 2.530(0.5); 2.522(0.5); 2.517(0.5); 2.512(0.6); 2.506(0.7);
2.499(1.0); 2.494(0.8); 2.492(0.7); 2.487(0.6); 2.481(0.6); 2.477(0.8); 2.468(0.6); 2.463(0.6); 2.459(0.3); 2.446(0.4); 2.441(0.3); 2.360(0.5); 2.358(0.5); 2.346(0.8); 2.333(0.9); 2.327(0.6); 2.318(1.1);
2.309(0.5); 2.303(0.7); 2.289(0.7); 2.278(0.3); 1.684(16.0); 0.013(0.4); 0.008(1.6); 0.000(40.0); −0.006(1.1); −0.008(1.5)
Example 726: ¹H-NMR(399.8 MHz, cdcl3):
δ = 7.760(5.2); 7.276(7.2); 7.260(37.3); 7.256(11.4); 7.083(2.5); 7.063(3.2); 7.043(1.7); 6.746(1.6); 6.611(3.3); 6.505(0.7); 6.475(1.8); 5.155(0.8); 5.147(0.7); 4.632(0.6); 4.610(1.8); 4.588(1.7); 4.567(0.6);
3.860(16.0); 3.210(0.4); 3.188(0.8); 3.169(0.9); 3.159(0.8); 3.144(0.7); 3.140(0.8); 3.137(0.7); 3.114(0.4); 2.586(0.4); 2.565(0.9); 2.555(0.5); 2.545(1.2); 2.535(1.0); 2.526(0.6); 2.515(1.1); 2.500(1.0);
2.494(0.8); 2.485(0.5); 2.479(0.6); 2.473(0.9); 2.468(0.9); 2.462(0.8); 2.455(0.5); 2.451(0.6); 2.445(0.7); 2.439(0.6); 2.433(0.5); 2.263(0.4); 2.250(0.8); 2.238(0.7); 2.233(0.7); 2.221(1.1); 2.213(0.6);
2.208(0.7); 2.196(0.7); 2.186(0.4); 1.665(10.0); 0.007(1.7); −0.001(38.0); −0.009(1.5)
Example 727: ¹H-NMR(399.8 MHz, cdcl3):
δ = 7.519(0.4); 7.337(0.8); 7.317(2.5); 7.309(16.0); 7.302(3.3); 7.296(3.4); 7.289(20.6); 7.280(5.5); 7.275(1.9); 7.260(60.7); 7.252(0.6); 7.250(0.5); 7.249(0.4); 7.248(0.4); 7.244(1.5); 7.112(6.1); 7.092(7.1);
7.083(0.3); 7.072(4.1); 6.868(1.1); 6.864(1.6); 6.858(8.2); 6.846(1.4); 6.843(2.0); 6.838(11.4); 6.829(1.2); 6.817(7.1); 6.811(1.2); 6.808(0.9); 6.266(1.3); 6.251(1.3); 5.288(0.5); 5.267(1.5); 5.255(1.6);
5.250(1.7); 5.246(1.6); 5.234(1.5); 5.213(0.5); 4.776(1.4); 4.753(3.7); 4.731(3.6); 4.708(1.3); 3.179(0.7); 3.176(0.7); 3.160(0.9); 3.158(1.3); 3.153(1.2); 3.150(1.2); 3.146(1.0); 3.134(1.3); 3.131(2.1);
3.128(2.0); 3.124(1.2); 3.122(1.2); 3.120(1.0); 3.113(1.0); 3.109(1.3); 3.106(1.2); 3.104(1.2); 3.101(1.5); 3.082(0.9); 3.080(0.9); 2.668(0.8); 2.648(1.9); 2.638(1.1); 2.628(1.9); 2.617(2.3); 2.608(1.1);
2.597(2.2); 2.578(1.2); 2.504(0.8); 2.499(0.9); 2.490(0.9); 2.486(1.0); 2.480(1.2); 2.475(1.3); 2.468(1.7); 2.462(1.5); 2.460(1.4); 2.455(1.3); 2.451(1.0); 2.449(1.1); 2.445(1.5); 2.439(1.1); 2.438(1.2);
2.436(1.3); 2.433(1.1); 2.431(1.1); 2.428(0.6); 2.423(0.5); 2.414(0.7); 2.409(0.7); 2.352(1.0); 2.350(1.0); 2.337(1.7); 2.325(1.8); 2.323(2.0); 2.310(2.1); 2.299(1.0); 2.295(1.6); 2.292(1.5); 2.280(1.3);
2.269(0.6); 2.266(0.6); 1.553(48.9); 1.255(1.0); 0.012(0.4); 0.008(2.1); 0.000(60.7); −0.009(2.1); −0.013(0.4); −0.014(0.3)
Example 728: ¹H-NMR(399.8 MHz, cdcl3):
δ = 7.534(8.4); 7.517(10.9); 7.513(8.5); 7.448(1.1); 7.445(1.9); 7.442(1.3); 7.432(1.6); 7.427(5.7); 7.422(1.9); 7.412(2.8); 7.409(4.5); 7.405(2.3); 7.360(8.2); 7.341(11.1); 7.327(2.2); 7.323(4.6); 7.312(16.0);
7.292(19.3); 7.260(41.3); 7.090(5.6); 7.070(7.2); 7.050(4.0); 6.532(1.4); 6.515(1.5); 5.252(0.6); 5.230(1.7); 5.217(1.8); 5.209(1.8); 5.196(1.7); 5.175(0.6); 4.856(1.4); 4.834(3.5); 4.811(3.5); 4.788(1.3);
3.217(0.7); 3.197(1.3); 3.188(1.2); 3.170(2.1); 3.167(2.0); 3.160(1.1); 3.149(1.3); 3.146(1.2); 3.140(1.4); 3.120(0.8); 2.712(0.8); 2.691(1.8); 2.681(1.2); 2.673(1.8); 2.659(2.3); 2.652(1.1); 2.641(2.1);
2.620(1.1); 2.496(0.7); 2.492(0.8); 2.482(0.9); 2.478(1.0); 2.468(1.2); 2.457(1.5); 2.454(1.4); 2.451(1.4); 2.447(1.4); 2.440(1.1); 2.436(1.3); 2.426(1.4); 2.415(0.5); 2.404(0.8); 2.400(0.7); 2.372(1.1);
2.358(1.7); 2.343(1.9); 2.329(2.1); 2.315(1.6); 2.312(1.5); 2.300(1.3); 2.285(0.6); 1.565(44.1); 1.255(0.7); 0.008(1.8); 0.000(42.2); −0.008(1.8)
Example 729: ¹H-NMR(399.8 MHz, cdcl3):
δ = 7.519(0.4); 7.497(3.6); 7.493(6.4); 7.488(4.1); 7.409(2.2); 7.407(2.7); 7.404(2.4); 7.401(2.2); 7.390(3.0); 7.387(4.0); 7.384(3.6); 7.382(5.2); 7.379(3.5); 7.375(2.2); 7.362(3.6); 7.359(4.5); 7.356(2.6);
7.321(12.7); 7.301(16.0); 7.295(5.7); 7.275(7.1); 7.260(61.1); 7.106(4.6); 7.086(5.6); 7.065(3.2); 6.996(0.3); 6.485(1.2); 6.468(1.2); 5.221(0.4); 5.200(1.3); 5.187(1.4); 5.179(1.4); 5.165(1.4); 5.157(0.6);
5.144(0.5); 4.867(1.1); 4.844(2.7); 4.822(2.6); 4.799(1.0); 3.222(0.5); 3.202(1.0); 3.192(0.9); 3.180(0.8); 3.174(1.6); 3.171(1.5); 3.164(0.8); 3.153(1.0); 3.150(0.9); 3.144(1.1); 3.126(0.7); 2.720(0.6);
2.699(1.4); 2.689(0.9); 2.681(1.4); 2.667(1.7); 2.660(0.9); 2.650(1.6); 2.628(0.9); 2.501(0.6); 2.497(0.6); 2.486(0.6); 2.482(0.7); 2.476(0.9); 2.461(1.2); 2.455(1.1); 2.451(1.0); 2.444(0.9); 2.440(0.9);
2.430(1.1); 2.419(0.4); 2.409(0.6); 2.405(0.6); 2.373(0.8); 2.359(1.3); 2.344(1.5); 2.330(1.6); 2.316(1.2); 2.313(1.1); 2.301(1.0); 2.286(0.5); 1.550(33.3); 1.255(0.4); 0.008(2.5); 0.000(62.7); −0.008(2.1)
Example 730: ¹H-NMR(399.8 MHz, cdcl3):
δ = 7.677(1.9); 7.656(2.6); 7.595(3.3); 7.559(2.0); 7.538(1.5); 7.260(20.8); 6.319(1.0); 6.304(1.0); 4.821(1.0); 4.799(1.5); 4.778(1.0); 3.719(0.5); 3.696(1.3); 3.674(1.3); 3.651(0.5); 2.713(16.0); 2.489(0.5);
2.468(1.2); 2.445(1.3); 2.424(0.6); 2.344(0.5); 2.320(1.3); 2.296(1.3); 2.273(0.6); 2.005(0.4); 1.979(1.1); 1.955(1.6); 1.931(1.0); 1.906(0.4); 1.838(0.5); 1.812(1.1); 1.790(1.3); 1.764(0.9); 1.546(24.7);
1.256(0.6); 0.000(22.3)
Example 731: ¹H-NMR(399.8 MHz, cdcl3):
δ = 7.691(1.8); 7.686(2.0); 7.619(0.5); 7.614(0.4); 7.598(1.9); 7.593(1.9); 7.583(2.8); 7.562(0.7); 7.262(7.3); 5.772(0.4); 5.762(0.4); 4.922(0.5); 4.904(0.7); 4.886(0.5); 4.382(0.3); 4.327(0.6); 4.308(0.6);
2.758(2.2); 2.664(16.0); 2.648(0.4); 2.643(0.5); 2.639(0.5); 2.636(0.5); 2.624(0.5); 2.621(0.5); 2.436(0.5); 2.419(0.7); 2.412(0.7); 2.404(0.5); 2.395(1.5); 2.378(0.9); 2.375(0.9); 2.152(0.5); 2.136(0.5);
2.126(0.5); 2.124(0.5); 2.121(0.5); 2.112(0.4); 2.096(0.3); 1.895(0.4); 1.559(2.7); 1.255(0.4); 0.008(0.4); 0.000(8.1)

### Complementary NMR-Data for:

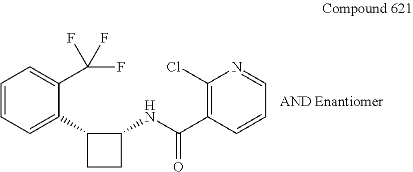 [SMILES: O=C(N[C@@H]1CC[C@@H]1c1ccccc1C(F)(F)F)c1cccnc1Cl]

¹H-NMR: 400 MHz, CDCl₃, δ, 8.37 (m, 1H), 8.05 (m, 1H), 7.70 (m, 1H), 7.66 (m, 2H), 7.28 (m, 2H), 6.37 (d, 1H), 5.03 (m, 1H), 4.43 (m, 1H), 2.69 (m, 1H), 2.49 (m, 2H), 2.22 (m, 1H)

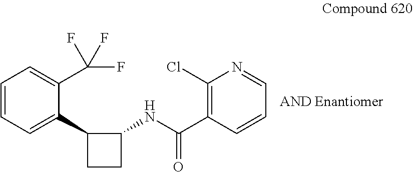 [SMILES: O=C(N[C@@H]1CC[C@H]1c1ccccc1C(F)(F)F)c1cccnc1Cl]

¹H-NMR: 400 MHz, CDCl₃, δ, 8.45 (m, 1H), 8.08 (m, 1H), 7.78 (m, 1H), 7.63 (m, 2H), 7.36 (m, 2H), 6.75 (d, 1H), 4.95 (m, 1H), 3.78 (m, 1H), 2.49 (m, 1H), 2.32 (m, 1H), 1.99 (m, 1H), 1.87 (m, 1H)

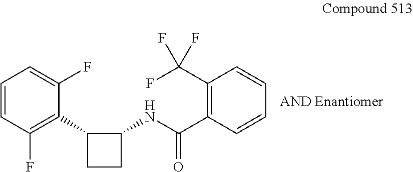 [SMILES: O=C(N[C@@H]1CC[C@@H]1c1c(F)cccc1F)c1ccccc1C(F)(F)F]

¹H-NMR: 400 MHz, DMSO-d₆, δ, 8.69 (m, 1H), 7.67 (m, 1H), 7.56 (m, 2H), 7.27 (m, 1H), 7.02 (m, 2H), 6.85 (m, 1H), 4.81 (m, 1H), 4.20 (m, 1H), 2.20-2.60 (m, 4H)

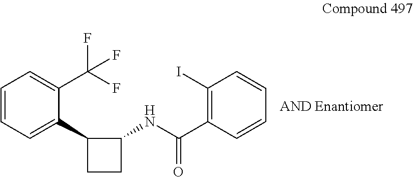 [SMILES: O=C(N[C@@H]1CC[C@H]1c1ccccc1C(F)(F)F)c1ccccc1I]

¹H-NMR: 400 MHz, CDCl₃, δ, 7.84 (m, 2H), 7.62 (m, 2H), 7.35 (m, 2H), 7.08 (m, 2H), 6.00 (d, 1H), 4.97 (m, 1H), 3.75 (m, 1H), 2.50 (m, 1H), 2.30 (m, 1H), 1.99 (m, 1H), 1.80 (m, 1H)

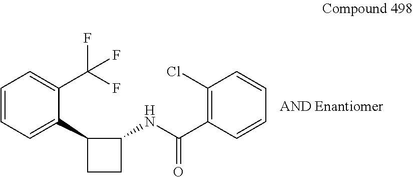 [SMILES: O=C(N[C@@H]1CC[C@H]1c1ccccc1C(F)(F)F)c1ccccc1Cl]

¹H-NMR: 400 MHz, CDCl₃, δ, 7.77 (m, 1H), 7.61 (m, 3H), 7.37 (m, 4H), 6.40 (d, 1H), 4.96 (m, 1H), 3.74 (m, 1H), 2.48 (m, 1H), 2.30 (m, 1H), 1.96 (m, 1H), 1.81 (m, 1H)

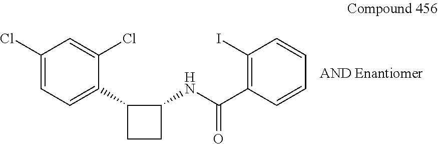 [SMILES: O=C(N[C@@H]1CC[C@@H]1c1ccc(Cl)cc1Cl)c1ccccc1I]

¹H-NMR: 400 MHz, CDCl₃, δ, 7.79 (m, 1H), 7.42-7.28 (m, 4H), 7.13 (m, 1H), 7.10 (m, 1H), 5.44 (d, 1H), 5.09 (m, 1H), 4.23 (m, 1H), 2.60 (m, 1H), 2.40 (m, 2H), 2.11 (m, 1H)

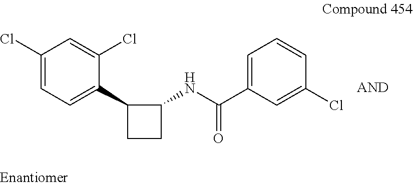 [SMILES: O=C(N[C@@H]1CC[C@H]1c1ccc(Cl)cc1Cl)c1cccc(Cl)c1]

¹H-NMR: 400 MHz, CDCl₃, δ, 7.74 (m, 1H), 7.27-7.53 (m, 6H), 6.37 (d, 1H), 4.81 (m, 1H), 3.69 (m, 1H), 2.36 (m, 2H), 2.00 (m, 1H), 1.69 (m, 1H)

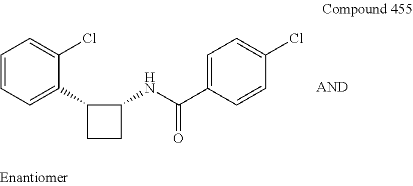 [SMILES: O=C(N[C@@H]1CC[C@@H]1c1ccccc1Cl)c1ccc(Cl)cc1]

¹H-NMR: 400 MHz, CDCl₃, δ, 7.51-7.26 (m, 8H), 5.61 (d, 1H), 5.07 (m, 1H), 4.30 (m, 1H), 2.60 (m, 1H), 2.38 (m, 2H), 2.10 (m, 1H)

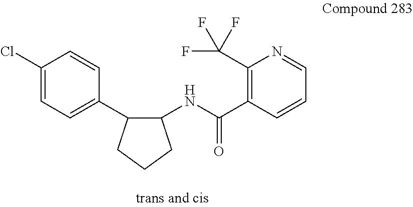 [SMILES: O=C(NC1CCCC1c1ccc(Cl)cc1)c1cccnc1C(F)(F)F]

¹H-NMR: mixture 78/22 of a major and a minor isomer

Major isomer: 400 MHz, CDCl₃, δ, 8.69-8.67 (m, 1H), 7.78-7.16 (m, 6H), 5.27 (d, 1H), 4.78 (m, 1H), 3.45 (m, 1H), 2.26 (m, 2H), 2.02-1.62 (m, 4H)

Minor isomer: 400 MHz, CDCl₃, δ, 8.69-8.67 (m, 1H), 7.78-7.16 (m, 6H), 5.70 (d, 1H), 4.46 (m, 1H), 2.91 (m, 1H), 2.32 (m, 1H), 2.26-1.62 (m, 5H)

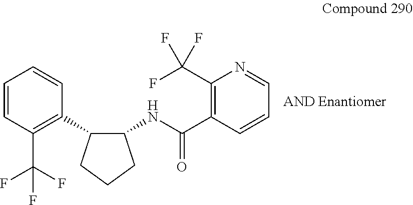 [SMILES: O=C(N[C@@H]1CCC[C@@H]1c1ccccc1C(F)(F)F)c1cccnc1C(F)(F)F]

¹H-NMR: 400 MHz, DMSO-d₆, δ, 8.73 (d, 1H), 8.51 (d, 1H), 7.72-7.61 (m, 4H), 7.44 (m, 1H), 7.30 (m, 1H), 4.61 (m, 1H), 3.47 (m, 1H), 2.21 (m, 1H), 2.06 (m, 1H), 1.92 (m, 2H), 1.71 (m, 2H)

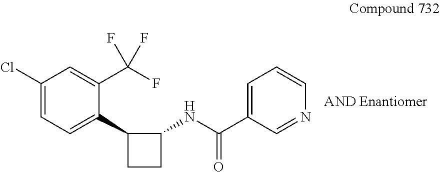 [SMILES: O=C(N[C@@H]1CC[C@H]1c1ccc(Cl)cc1C(F)(F)F)c1cccnc1]

¹H-NMR: 400 MHz, CDCl₃, δ, 8.93 (m, 1H), 8.72 (m, 1H), 8.06 (m, 1H), 7.72 (m, 1H), 7.59 (m, 2H), 7.55 (m, 1H), 6.38 (d, 1H), 4.89 (m, 1H), 3.74 (m, 1H), 2.47 (m, 1H), 2.29 (m, 1H), 1.99 (m, 1H), 1.83 (m, 1H)

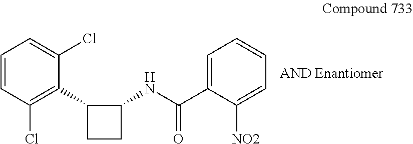 [SMILES: O=C(N[C@@H]1CC[C@@H]1c1c(Cl)cccc1Cl)c1ccccc1[N+](=O)[O-]]

¹H-NMR: 400 MHz, CDCl₃, δ, 7.99 (m, 1H), 7.51 (m, 2H), 7.32 (m, 2H), 7.12 (m, 1H), 7.06 (m, 1H), 6.11 (d, 1H), 5.23 (m, 1H), 4.79 (m, 1H), 3.18 (m, 1H), 2.66 (m, 1H), 2.63 (m, 1H), 2.34 (m, 1H),

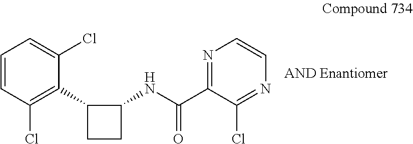 [SMILES: O=C(N[C@@H]1CC[C@@H]1c1c(Cl)cccc1Cl)c1nccnc1Cl]

¹H-NMR: 400 MHz, CDCl₃, δ, 8.44 (d, 1H), 8.37 (m, 1H), 7.25-6.04 (m, 4H), 5.21 (m, 1H), 4.80 (m, 1H), 3.19 (m, 1H), 2.66 (m, 1H), 2.48 (m, 1H), 2.37 (m, 1H)

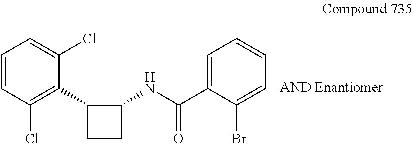 [SMILES: O=C(N[C@@H]1CC[C@@H]1c1c(Cl)cccc1Cl)c1ccccc1Br]

¹H-NMR: 400 MHz, CDCl₃, δ, 7.49 (m, 1H), 7.28-7.07 (m, 6H), 6.30 (d, 1H), 5.23 (m, 1H), 4.72 (m, 1H), 3.16 (m, 1H), 2.63 (m, 1H), 2.58 (m, 1H), 2.47 (m, 1H),

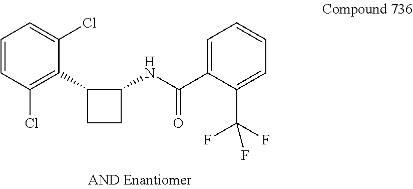 [SMILES: O=C(N[C@@H]1CC[C@@H]1c1c(Cl)cccc1Cl)c1ccccc1C(F)(F)F]

¹H-NMR: 400 MHz, CDCl₃, δ, 7.61 (m, 1H), 7.47-7.08 (m, 6H), 6.06 (d, 1H), 5.21 (m, 1H), 4.70 (m, 1H), 3.10 (m, 1H), 2.47 (m, 1H), 2.45 (m, 1H), 2.27 (m, 1H),

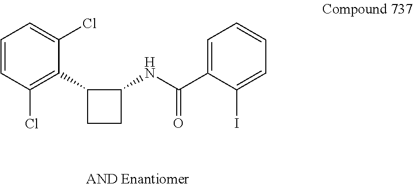 [SMILES: O=C(N[C@@H]1CC[C@@H]1c1c(Cl)cccc1Cl)c1ccccc1I]

¹H-NMR: 400 MHz, CDCl₃, δ, 7.78 (m, 1H), 7.30-6.90 (m, 6H), 6.10 (d, 1H), 5.21 (m, 1H), 4.75 (m, 1H), 3.17 (m, 1H), 2.60 (m, 1H), 2.37 (m, 1H), 2.33 (m, 1H),

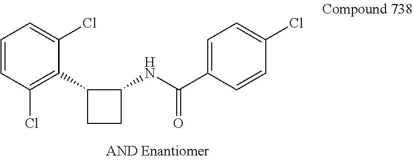 [SMILES: O=C(N[C@@H]1CC[C@@H]1c1c(Cl)cccc1Cl)c1ccc(Cl)cc1]

¹H-NMR: 400 MHz, CDCl₃, δ, 7.47 (d, 2H), 7.28 (m, 4H), 7.00 (t, 1H), 6.49 (d, 1H), 5.17 (m, 1H), 4.82 (m, 1H), 3.16 (m, 1H), 2.69 (m, 1H), 2.64 (m, 1H), 2.45 (m, 1H)

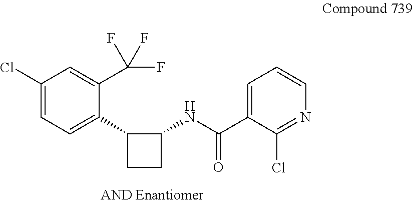 [SMILES: O=C(N[C@@H]1CC[C@@H]1c1ccc(Cl)cc1C(F)(F)F)c1cccnc1Cl]

¹H-NMR: 400 MHz, CDCl₃, δ, 8.39 (m, 1H), 8.06 (m, 1H), 7.68 (m, 2H), 7.28 (m, 2H), 6.34 (d, 1H), 5.03 (m, 1H), 4.37 (m, 1H), 2.69 (m, 1H), 2.44 (m, 2H), 2.16 (m, 1H),

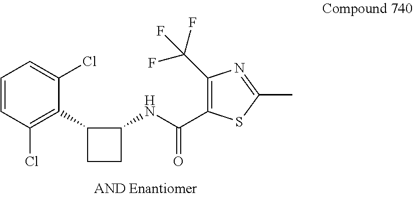 [SMILES: Cc1nc(C(F)(F)F)c(C(=O)N[C@@H]2CC[C@@H]2c2c(Cl)cccc2Cl)s1]

¹H-NMR: 400 MHz, CDCl₃, δ, 7.31 (m, 2H), 7.09 (m, 1H), 6.39 (d, 1H), 5.13 (m, 1H), 4.68 (m, 1H), 3.15 (m, 1H), 2.67 (s, 3H), 2.59 (m, 1H), 2.46 (m, 1H), 2.29 (m, 1H)

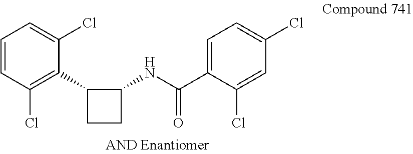 [SMILES: O=C(N[C@@H]1CC[C@@H]1c1c(Cl)cccc1Cl)c1ccc(Cl)cc1Cl]

¹H-NMR: 400 MHz, CDCl₃, δ, 7.42-6.51 (m, 6H), 6.49 (d, 1H), 5.21 (m, 1H), 4.72 (m, 1H), 3.15 (m, 1H), 2.61 (m, 1H), 2.58 (m, 1H), 2.29 (m, 1H),

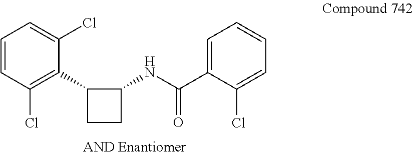 [SMILES: O=C(N[C@@H]1CC[C@@H]1c1c(Cl)cccc1Cl)c1ccccc1Cl]

¹H-NMR: 400 MHz, CDCl₃, δ,

7.42-7.07 (m, 7H), 6.48 (d, 1H), 5.24 (m, 1H), 4.74 (m, 1H), 3.16 (m, 1H), 2.48 (m, 1H), 2.44 (m, 1H), 2.30 (m, 1H)

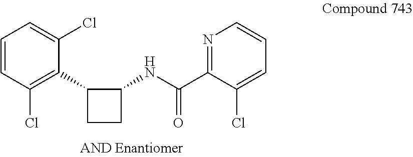 [SMILES: O=C(N[C@@H]1CC[C@@H]1c1c(Cl)cccc1Cl)c1ncccc1Cl]

¹H-NMR: 400 MHz, CDCl₃, δ, 8.36 (d, 1H), 8.34 (m, 1H), 7.70 (d, 1H), 7.24 (m, 2H), 7.00 (m, 1H), 5.20 (m, 1H), 4.74 (m, 1H), 3.21 (m, 1H), 2.58 (m, 1H), 2.46 (m, 1H), 2.36 (m, 1H)

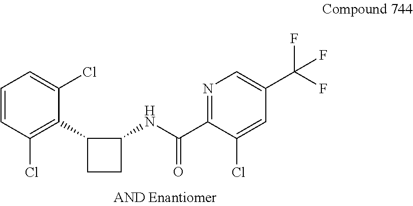 [SMILES: O=C(N[C@@H]1CC[C@@H]1c1c(Cl)cccc1Cl)c1ncc(C(F)(F)F)cc1Cl]

¹H-NMR: 400 MHz, CDCl₃, δ, 8.61 (m, 1H), 8.00 (s, 1H), 7.96 (s, 1H), 7.70 (m, 1H), 7.05 (m, 1H), 5.19 (m, 1H), 4.79 (m, 1H), 3.17 (m, 1H), 2.62 (m, 1H), 2.45 (m, 1H), 2.34 (m, 1H)

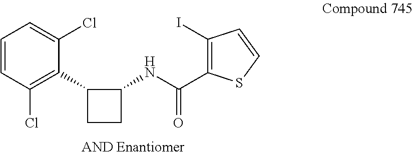 [SMILES: O=C(N[C@@H]1CC[C@@H]1c1c(Cl)cccc1Cl)c1sccc1I]

¹H-NMR: 400 MHz, CDCl₃, δ, 7.32-7.27 (m, 4H), 7.10-7.06 (m, 2H), 5.20 (m, 1H), 4.58 (m, 1H), 3.26 (m, 1H), 2.51 (m, 2H), 2.24 (m, 1H)

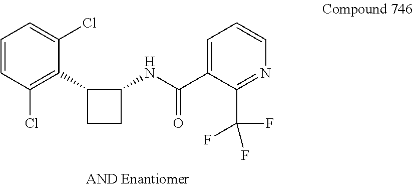 [SMILES: O=C(N[C@@H]1CC[C@@H]1c1c(Cl)cccc1Cl)c1cccnc1C(F)(F)F]

¹H-NMR: 400 MHz, CDCl₃, δ, 8.70 (m, 1H), 7.49 (m, 2H), 7.44 (m, 2H), 7.10 (m, 1H), 6.08 (d, 1H), 5.21 (m, 1H), 4.73 (m, 1H), 3.14 (m, 1H), 2.63 (m, 1H), 2.48 (m, 1H), 2.31 (m, 1H)

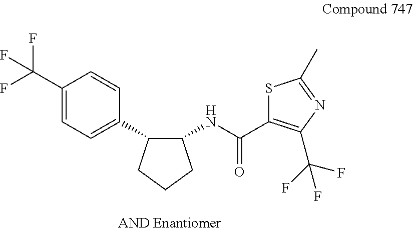 [SMILES: Cc1nc(C(F)(F)F)c(C(=O)N[C@@H]2CCC[C@@H]2c2ccc(C(F)(F)F)cc2)s1]

¹H-NMR: 400 MHz, CDCl₃, δ, 8.60 (d, 1H), 7.63 (d, 2H), 7.42 (d, 2H), 4.61 (m, 1H), 3.38 (m, 1H), 2.65 (s, 3H), 2.11 (m, 1H), 2.00 (m, 2H), 1.87 (m, 1H), 1.65 (m, 2H)

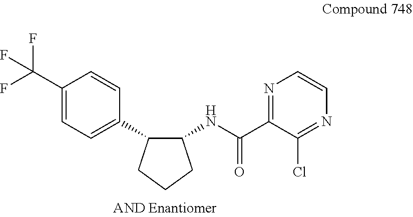 [SMILES: O=C(N[C@@H]1CCC[C@@H]1c1ccc(C(F)(F)F)cc1)c1nccnc1Cl]

¹H-NMR: 400 MHz, CDCl₃, δ, 8.60 (d, 1H), 8.53 (d, 1H), 8.31 (d, 1H), 7.59 (d, 1H), 7.48 (d, 1H), 4.72 (m, 1H), 3.38 (m, 1H), 2.32-1.76 (m, 4H), 1.23 (m, 2H)

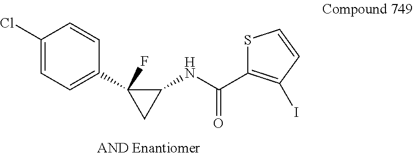 [SMILES: O=C(N[C@@H]1C[C@]1(F)c1ccc(Cl)cc1)c1sccc1I]

¹H-NMR: 400 MHz, CDCl₃, δ, 7.46-7.34 (m, 5H), 7.03 (s, 1H), 6.48 (s, 1H), 3.70 (dd, 1H), 2.00 (dd, 1H), 1.58 (m, 1H)

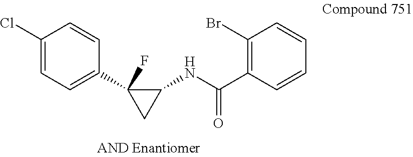 [SMILES: O=C(N[C@@H]1C[C@]1(F)c1ccc(Cl)cc1)c1ccccc1Br]

¹H-NMR: 400 MHz, CDCl₃, δ, 7.50-7.26 (m, 8H), 5.65 (s, 1H), 3.70 (dd, 1H), 1.99 (dd, 1H), 1.60 (m, 1H)

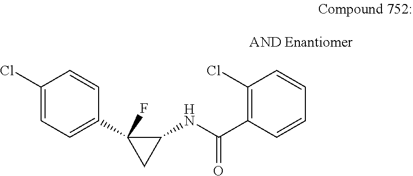 [SMILES: O=C(N[C@@H]1C[C@]1(F)c1ccc(Cl)cc1)c1ccccc1Cl]

¹H-NMR: 400 MHz, CDCl₃, δ, 7.49-7.27 (m, 8H), 5.89 (s, 1H), 3.69 (dd, 1H), 2.02 (dd, 1H), 1.57 (m, 1H)

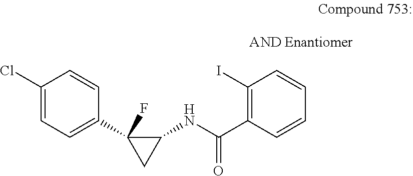 [SMILES: O=C(N[C@@H]1C[C@]1(F)c1ccc(Cl)cc1)c1ccccc1I]

¹H-NMR: 400 MHz, CDCl₃, δ, 7.79 (d, 1H), 7.77-7.26 (m, 5H), 7.03 (m, 2H), 5.40 (s, 1H), 3.69 (dd, 1H), 2.01 (dd, 1H), 1.57 (m, 1H)

The following examples illustrate in a non-limiting manner the preparation and efficacy of the compounds of formula (I) according to the invention.

### Preparation Example 1

### cis-3-chloro-N-[2-(2-chlorophenyl)cyclobutyl]pyridine-2-carboxamide (Compound 247) and trans-3-chloro-N-[2-(2-chlorophenyl)cyclobutyl]pyridine-2-carboxamide (Compound 238)

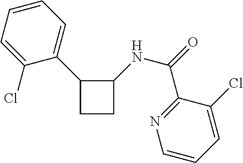 [SMILES: O=C(NC1CCC1c1ccccc1Cl)c1ncccc1Cl]

### Step 1: 2-[2-chlorophenyl]cyclobutanone

To a solution of 2-chlorobenzaldehyde (10.0 g, 1.0 eq) and cyclopropyl(diphenyl)sulfonium tetrafluoroborate (22.34 g, 1.1 eq) in THF (100 mL) was added t-BuOK (142 mL, 1M in THF) at −70° C. and stirred at −70° C. to 0° C. for 1 h. After work-up and column purification, 6.0 g of the title compound were isolated.

### Step 2: 2-(2-chlorophenyl)-N-hydroxycyclobutanimine

To a solution of 2-[2-chlorophenyl]cyclobutanone (6.0 g, 1.0 eq) in ethanol (60 mL) was added hydroxyl amine hydrochloride (3.46 g, 1.5 eq) followed by K₂CO₃ (9.16 g, 3 eq) at RT (room temperature) and stirred for 3 h. After work-up and column purification 3.0 g of the title compound were isolated.

### Step 3: 2-(2-chlorophenyl)cyclobutanamine

To a stirred solution of 2-(2-chlorophenyl)-N-hydroxycyclobutanimine (2.6 g, 1.0 eq) in THF (26 mL) was added Borane-THF-complex (3 mL, 2.0eq., 1M) at 0° C., stirred at RT for 3 h and at 65° C. for 16 h.

The reaction mixture was quenched with methanol (20 mL), refluxed for 3 h and the solvent was evaporated to obtain 2.10 g of crude title product as a diastereomeric mixture (50:50).

### Step 4: cis-3-chloro-N-[2-(2-chlorophenyl)cyclobutyl]pyridine-2-carboxamide (Compound 247) and trans-3-chloro-N-[2-(2-chlorophenyl)cyclobutyl]pyridine-2-carboxamide (Compound 238)

To a solution of 2-(2-chlorophenyl)cyclobutanamine (150 mg, 1.0 eq) in dichloromethane (DCM) (3 mL) was added triethylamine (0.34 mL, 3.0 eq), 3-chloropyridine-2-carboxylic acid (143 mg, 1.1 eq) followed by HATU (O-(7-azabenzotriazol-1-yl)-N,N,N′,N′-tetramethyluronium hexafluorophosphate) (470 mg, 1.5 eq) at RT and stirred at RT for 3 h. After work-up and column purification, the title compound was isolated as a cis/trans mixture. Both isomers were separated by preparative HPLC, yielding 55 mg (20.8%) of cis-3-chloro-N-[2-(2-chlorophenyl)cyclobutyl]pyridine-2-carboxamide and 50 mg (18.9%) of trans-3-chloro-N-[2-(2-chlorophenyl)cyclobutyl]pyridine-2-carboxamide.

cis-Isomer: HPLC-MS: log P=2.79; Mass (m/z): 321.0 (M+H)⁺; ¹H-NMR [CDCl₃] 2.09-2.16 (m, 1H), 2.32-2.39 (m, 1H), 2.42-2.48 (m, 1H), 2.57-2.63 (m, 1H), 4.30-4.36 (m, 1H), 5.03-5.09 (m, 1H), 7.21-7.23 (m, 1H), 7.34-7.41 (m, 3H), 7.48-7.49 (m, 1H), 7.98 (s, 1H), 8.47 (s, 1H).

trans-Isomer: HPLC-MS: log P=3.06; Mass (m/z): 321.0 (M+H)⁺; ¹H-NMR [CDCl₃] 1.66-1.76 (m, 1H), 1.98-2.08 (m, 1H), 2.39-2.47 (m, 2H), 3.77-3.85 (m, 1H), 4.86-4.95 (m, 1H), 7.13-7.17 (m, 1H), 7.28-7.38 (m, 3H), 7.59-7.61 (m, 1H), 7.80-7.82 (m, 1H), 8.20-8.22 (m, 1H), 8.45-8.46 (m, 1H).

### Preparation Example 2

### cis-N-[2-(2,4-dichlorophenyl)cyclobutyl]-3-(difluoromethyl)-1-methyl-1H-pyrazole-4-carboxamide and trans-N-[2-(2,4-dichlorophenyl)cyclobutyl]-3-(difluoromethyl)-1-methyl-1H-pyrazole-4-carboxamide

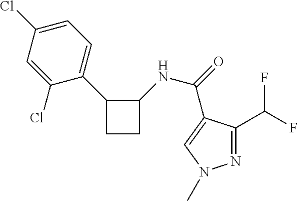 [SMILES: Cn1cc(C(=O)NC2CCC2c2ccc(Cl)cc2Cl)c(C(F)F)n1]

### Step 1: 2-(2,4-dichlorophenyl)cyclobutanone

To a solution of 2,4-dichlorobenzaldehyde (8.0 g, 1.0 eq) and cyclopropyl(diphenyl)sulfonium tetrafluoroborate (14.6 g, 1.1 eq) in THF (80 mL) was added t-BuOK (93 mL, 1M in THF) at −70° C. and stirred at −70° C. to 0° C. for 2 h. After work-up and column purification, 5.0 g of the title compound were isolated.

### Step 2: 2-(2,4-dichlorophenyl)-N-hydroxycyclobutanimine

To a solution of 2-(2,4-dichlorophenyl)cyclobutanone (5.0 g, 1.0 eq) in ethanol (50 mL) was added hydroxyl amine hydrochloride (2.43 g, 1.5 eq) followed by K₂CO₃ (9.67 g, 3 eq) at RT and stirred for 16 h. After work-up and column purification 2.7 g of the title compound were isolated.

### Step 3: 2-(2,4-dichlorophenyl)cyclobutanamine

To a stirred solution of 2-(2,4-dichlorophenyl)-N-hydroxycyclobutanimine (1.0 g, 1.0 eq) in THF (10 mL) was added Borane-THF-complex (8.7 mL, 2.0eq., 1M) at 0° C., stirred at RT for 3 h and at 65° C. for 16 h. The reaction mixture was quenched with methanol (3 mL), refluxed for 3 h and the solvent was evaporated to obtain 800 mg of crude title product as a diastereomeric mixture (50:50).

### Step 4: ds-N-[2-(2,4-dichlorophenyl)cyclobutyl]-3-(difluoromethyl)-1-methyl-1H-pyrazole-4-carboxamide and trans-N-[2-(2,4-dichlorophenyl)cyclobutyl]-3-(difluoromethyl)-1-methyl-1H-pyrazole-4-carboxamide

To a solution of 2-(2,4-dichlorophenyl)cyclobutanamine (150 mg, 1.0 eq) in DCM (3 mL) was added triethylamine (0.29 mL, 3.0 eq) and 3-(difluoromethyl)-1-methyl-1H-pyrazole-4-carbonyl chloride (149 mg, 1.1 eq) at RT and stirred at RT for 1 h. After work-up and column purification the title compound was isolated as a cis/trans mixture of isomers. Both isomers were separated by preparative HPLC, yielding 70 mg (27%) of cis-N-[2-(2,4-dichlorophenyl)cyclobutyl]-3-(difluoromethyl)-1-methyl-1H-pyrazole-4-carboxamide and 110 mg (42%) of trans-N-[2-(2,4-dichlorophenyl)cyclobutyl]-3-(difluoromethyl)-1-methyl-1H-pyrazole-4-carboxamide.

cis-Isomer: HPLC-MS: log P=2.95; Mass (m/z): 374.0 (M+H)⁺; ¹H-NMR [CDCl₃] 1.99-2.06 (m, 1H), 2.27-2.40 (m, 2H), 2.53-2.60 (m, 1H), 3.85 (s, 3H), 4.18-4.24 (m, 1H), 5.02-5.05 (m, 1H), 6.04-6.06 (m, 1H), 6.53 (t, 1H), 7.28-7.37 (m, 3H), 7.78 (s, 1H).

trans-Isomer: HPLC-MS: log P=3.26; Mass (m/z): 374.0 (M+H)⁺; ¹H-NMR [CDCl₃] 1.62-1.69 (m, 1H), 1.89-1.99 (m, 1H), 2.34-2.44 (m, 2H), 3.61-3.69 (1H), 3.91 (s, 3H), 4.77-4.81 (m, 1H), 6.60-6.62 (m, 1H), 6.81 (t, 1H), 7.22-7.25 (m, 1H), 7.32-7.33 (m, 1H), 7.51-7.53 (m, 1H), 7.89 (s, 1H).

### Preparation Example 3

### cis-N-[2-(2,6-difluorophenyl)cyclobutyl]-2-(trifluoromethyl)nicotinamide (Compound 236)

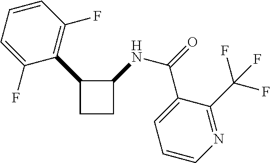

### Step 1: 2-(2,6-difluorophenyl)cyclobutanone

To a solution of 2,6-difluorobenzaldehyde (5.0 g, 1.0 eq) and cyclopropyl(diphenyl)sulfonium tetrafluoroborate (12.17 g, 1.1 eq) in THF (50 mL) was added t-BuOK (71.8 mL, 1M in THF) at −70° C. and stirred at −70° C. to −50° C. for 1 h. After work-up and column purification, 1.60 g (23.4%) of the title compound were isolated in a purity of 52%. The crude material was submitted to the next step without further purification.

### Step 2: 2-(2,6-difluorophenyl)-N-hydroxycyclobutanimine

To a solution of 2-(2,6-difluorophenyl)cyclobutanone (1.90 g, 1.0 eq) in ethanol (20 mL) was added hydroxyl amine hydrochloride (1.09 g, 1.5 eq) followed by K₂CO₃ (4.3 g, 3 eq) at RT and stirred for 16 h. After work-up 800 mg of the title compound were isolated.

### Step 3: 2-(2,6-difluorophenyl)cyclobutanamine

To a solution of 2-(2,6-difluorophenyl)-N-hydroxycyclobutanimine (700 mg, 1.0 eq) in a mixture of ethanol (7 mL) and chloroform (0.7 mL) was added PtO₂ (100 mg) and the reaction mixture was hydrogenated under balloon hydrogen pressure at RT for 18 h. After work-up and purification, 300 mg of the title compound were isolated. The crude material was submitted to the next step without further purification.

### Step 4: cis-N-[2-(2,6-difluorophenyl)cyclobutyl]-2-(trifluoromethyl)nicotinamide (Compound 236)

To a solution of 2-(2,6-difluorophenyl)cyclobutanamine (200 mg, 1.0 eq) in DCM (4 mL) was added triethylamine (0.45 mL, 3.0 eq), 3-(trifluoromethyl)pyridine-2-carboxylic acid (229 mg, 1.1 eq) followed by HATU (622 mg, 1.5 eq) at RT and stirred at RT for 3 h. After work-up and column purification, 80 mg (20.5%) of the title compound were isolated. HPLC-MS: log P=2.60; Mass (m/z): 355.0 (M+H)⁺; ¹H-NMR [CDCl₃] 2.31-2.38 (m, 1H), 2.42-2.53 (m, 2H), 2.64-2.69 (m, 1H), 4.37-4.43 (m, 1H), 5.09-5.12 (m, 1H), 6.01-6.03 (m, 1H), 6.87-6.93 (m, 2H), 7.18-7.24 (m, 1H), 7.41-7.46 (m, 2H), 8.69-8.71 (m, 1H).

### Preparation Example 4

### cis-2-chloro-N-{2-[2-(trifluoromethyl)phenyl]cyclobutyl}benzamide

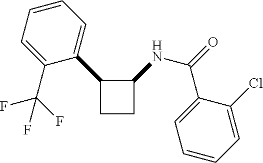

### Step 1: 2-[2-(trifluoromethyl)phenyl]cyclobutanone

To a solution of 2-(trifluoromethyl)benzaldehyde (5.0 g, 1.0 eq) and cyclopropyl(diphenyl)sulfonium tetrafluoroborate (10.8 g, 1.1 eq) in THF (50 mL) was added t-BuOK (71.8 mL, 1M in THF) at 0° C. and stirred at 0° C. to RT for 30 min. After work-up and column purification, 2.7 g of the title compound were isolated.

### Step 2: N-hydroxy-2-[2-(trifluoromethyl)phenyl]cyclobutanimine

To a solution of 2-[2-(trifluoromethyl)phenyl]cyclobutanone (2.7 g, 1.0 eq) in ethanol (27 mL) was added hydroxyl amine hydrochloride (643 mg, 1.5 eq) followed by K₂CO₃ (5.2 g, 3 eq) at RT and stirred for 2 h. After work-up and column purification 1.4 g of the title compound were isolated.

### Step 3: 2-[2-(trifluoromethyl)phenyl]cyclobutanamine

To a solution of N-hydroxy-2-[2-(trifluoromethyl)phenyl]cyclobutanimine (1.4 g, 1.0 eq) in a mixture of ethanol (14 mL) and chloroform (2 mL) was added PtO₂ (140 mg) and the reaction mixture was hydrogenated under balloon hydrogen pressure at RT for 18 h. After work-up and purification, 560 mg of the title compound were isolated as a diastereomeric mixture (80:20).

### Step 4: cis-2-chloro-N-{2-[2-(trifluoromethyl)phenyl]cyclobutyl}benzamide

To a solution of 2-[2-(trifluoromethyl)phenyl]cyclobutanamine (80 mg, 1.0 eq) in DCM (2 mL) was added triethylamine (0.15 mL, 3.0 eq), 2-chlorobenzoic acid (64 mg, 1.1 eq) followed by HATU (212 mg, 1.5 eq) at RT and stirred at RT for 5 h. After work-up and column purification, 40 mg (30.5%) of the title compound were isolated. HPLC-MS: log P=3.30; Mass (m/z): 354.0 (M+H)⁺; ¹H-NMR [CDCl₃] 2.18-2.23 (m, 1H), 2.33-2.38 (m, 2H), 2.40-2.45 (m, 1H), 4.38-4.42 (m, 1H), 5.03-5.07 (m, 1H), 6.05 (s, br, 1H), 7.19-7.25 (m, 3H), 7.34-7.40 (m, 1H), 7.52-7.55 (m, 1H), 7.60-7.64 (m, 1H), 7.69-7.71 (m, 2H).

### Preparation Example 5

### cis-2-iodo-N-{2-[2-(trifluoromethyl)phenyl]cyclobutyl}benzamide

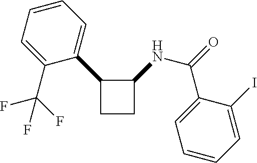

To a solution of 2-[2-(trifluoromethyl)phenyl]cyclobutanamine (50 mg, 1.0 eq as a diastereomeric mixture 80:20) in DCM (2 mL) was added triethylamine (0.1 mL, 3.0 eq), 2-iodobenzoic acid (63 mg, 1.2 eq) followed by HATU (132 mg, 1.5 eq) at RT and stirred at RT for 5 h. After work-up and column purification, 30 mg (29.1%) of title compound were isolated. HPLC-MS: log P=3.44; Mass (m/z): 446.0 (M+H)⁺; ¹H-NMR [CDCl₃] 2.20-2.25 (m, 1H), 2.35-2.48 (m, 2H), 2.63-2.70 (m, 1H), 4.38-4.42 (m, 1H), 4.99-5.04 (m, 1H), 5.43-5.45 (m, 1H), 6.98-7.03 (m, 2H), 7.22-7.26 (m, 1H), 7.37-7.41 (m, 1H), 7.59-7.76 (m, 4H).

### Preparation Example 6

### rel-N-[(1R,2S)-2-(4-chlorophenyl)-2-fluorocyclopropyl]-2,6-difluorobenzamide (Compound 750) and rel-N-[(1R,2R)-2-(4-chlorophenyl)-2-fluorocyclopyl]-2,6-difluorobenzamide

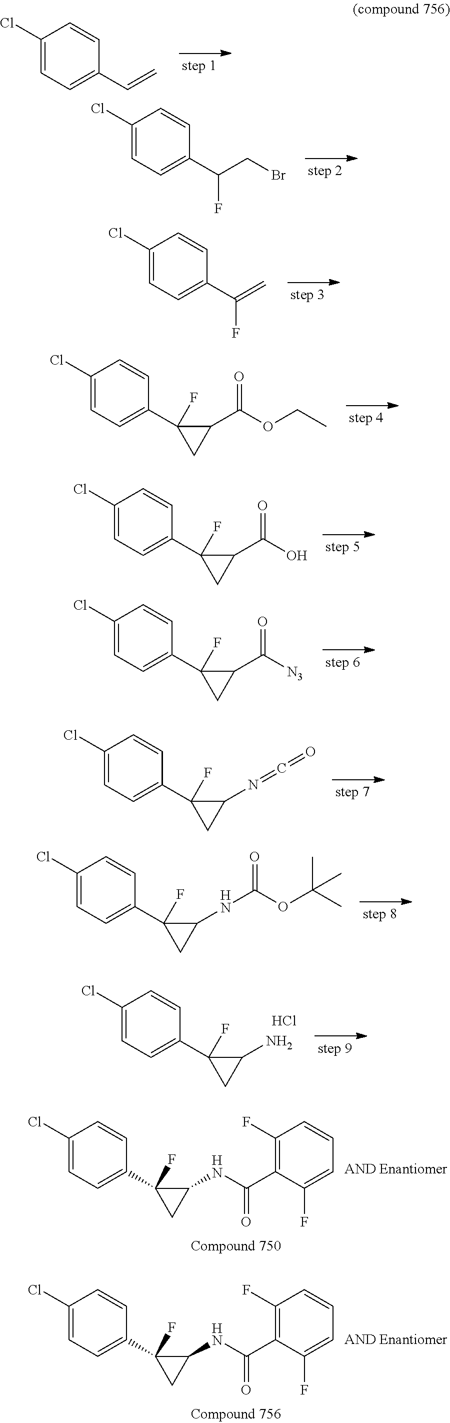

To a stirred solution of 1-chloro-4-vinylbenzene (25 g, 1.0 eq) and NBS (38.5 g, 1.2 eq) in dichloromethane (250 mL) was added triethylamine trihydrofluoride (35.2 mL, 1.2 eq) slowly drop wise at 0° C. The reaction mixture was stirred at room temperature for 16 h. After work-up 39 g of the title compound were isolated.

### Step 2: 1-chloro-4-(1-fluorovinyl)benzene

To a stirred solution of 1-(2-bromo-1-fluoroethyl)-4-chlorobenzene (39 g, 1.0 eq) in tetrahydrofurane (120 mL) was added t-BuOK (164.2 mL, 1.0 eq, 1 M in THF) at 0° C. and stirred at RT for 211. After work-up and purification by distillation, 15 g of the title compound were isolated.

### Step 3: ethyl 2-(4-chlorophenyl)-2-fluorocyclopropanecarboxylate

To a stirred solution of 1-chloro-4-(1-fluorovinyl)benzene (5 g, 1.0 eq) in DCM (30 mL) was added Cu(acac)₂ (251 mg, 0.03 eq) at room temperature. Ethyl diazoacetate (4.37 g, 1.2 eq) in dichloromethane (20 mL) was added slowly drop wise for 6 h to the reaction mixture at 40° C. and stirred at 40° C. for additional 2 h. After work-up and column purification 5 g of the title compound were isolated as a cis & trans (1:1) mixture

### Step 4: 2-(4-chlorophenyl)-2-fluorocyclopropanecarboxylic acid

To a stirred solution of ethyl 2-(4-chlorophenyl)-2-fluorocyclopropanecarboxylate (5 g, 1.0 eq) in methanol (150 mL) was added potassium hydroxide (11.5 g, 10.0 eq) slowly portionswise at 0-5° C. The reaction mixture was stirred at room temperature for 18 h. After work-up and purification 3 g of the title compound were isolated as a cis & trans (1:1) mixture.

### Step 5: [2-(4-chlorophenyl)-2-fluorocyclopropyl](2lambda5-triaz-1-en-2-yn-1-yl)methanone

To a stirred solution of 2-(4-chlorophenyl)-2-fluorocyclopropanecarboxylic acid (3 g, 1.0 eq) in tetrahydrofurane (30 mL) was added triethylamine (2.9 mL, 1.5 eq) followed DPPA (3.6 mL, 1.2 eq) at room temperature and stirred for 2 h. After work-up, isolated 3 g of the title compound were isolated and directly used in the next step.

### Step 6: 1-chloro-4-(1-fluoro-2-isocyanatocyclopropyl)benzene

The solution of [2-(4-chlorophenyl)-2-fluorocyclopropyl](2lambda5-triaz-1-en-2-yn-1-yl)methanone (3 g, 1.0 eq) in toluene (30 mL) was heated and stirred at 110° C. for 1 h. After solvent evaporation 3 g of the title compound were isolated and directly used in the next step.

### Step 7: tert-butyl[2-(4-chlorophenyl)-2-fluorocyclopropyl]carbamate

The solution of 1-chloro-4-(1-fluoro-2-isocyanatocyclopropyl)benzene (3 g, 1.0 eq) in t-BuOH (30 mL) was heated and stirred at 80° C. for 16 h. After work-up and column purification 2.8 g of the title compound were isolated as cis & trans mixture.

### Step 8: 2-(4-chlorophenyl)-2-fluorocyclopropanamine hydrochloride

To a stirred solution of tert-butyl[2-(4-chlorophenyl)-2-fluorocyclopropyl]carbamate (2.8 g, 1.0 eq) in methanol (20 mL) was added methanolic HCl (20 mL) at room temperature and stirred at 70° C. for 4 h. After work-up and purification 1.8 g of the title compound were isolated as cis & trans mixture.

### Step 9: rel-N-[(1R,2S)-2-(4-chlorophenyl)-2-fluorocyclopropyl]-2,6-difluorobenzamide (Compound 750) and rel-N-[(1R,2R)-2-(4-chlorophenyl)-2-fluorocyclopropyl]-2,6-difluorobenzamide (Compound 756)

To stirred solution of 2-(4-chlorophenyl)-2-fluorocyclopropanamine hydrochloride (150 mg, 1.0 eq) in dichloromethane (3 mL) was added 2,6-difluorobenzoic acid (117 mg, 1.1 eq), triethylamine (0.28 mL, 3.0 eq) followed by HATU (385 mg, 1.5 eq) at RT and stirred for 3 h. After work-up and preparative HPLC purification, isolated 65 mg of compound 750 and 70 mg of compound 756.

compound 750: HPLC-MS: log P^{b)}=2.69;

¹H-NMR: 400 MHz, CDCl₃, δ, 7.42-7.26 (m, 5H), 6.87 (m, 2H), 5.56 (s, 1H), 3.69 (dd, 1H), 2.01 (dd, 1H), 1.57 (m, 1H)

### Biological Examples

### Boonhilus micronlus—Injectiontest (BOOPMI Inj)

Solvent: dimethyl sulfoxide

To produce a suitable preparation of active compound, 10 mg of active compound are dissolved in 0.5 ml solvent, and the concentrate is diluted with solvent to the desired concentration.

Five adult engorged female ticks (Boophilus microplus) are injected with 1 μl compound solution into the abdomen. Ticks are transferred into replica plates and incubated in a climate chamber.

After 7 days the egg deposition of fertile eggs is monitored. Eggs where fertility is not visible are stored in a climate chamber till hatching after about 42 days. An efficacy of 100% means that all eggs are infertile; 0% means that all eggs are fertile.

In this test, for example, the following compounds from the preparation examples showed good activity of 80% at an application rate of 20 μg/animal: 49

Cooneria curtice—Test (COOPCU)
Solvent: dimethyl sulfoxide

To produce a suitable preparation of active compound, 10 mg of active compound are dissolved in 0.5 ml solvent, and the concentrate is diluted with “Ringer's solution” to the desired concentration.

Approximately 40 nematode larvae (Cooperia curticei) are transferred into a test tube containing the compound solution.

After 5 days percentage of larval mortality is recorded. 100% efficacy means all larvae are killed; 0% efficacy means no larvae are killed.

In this test for example, the following compounds from the preparation examples showed good activity of 100% at an application rate of 20 ppm: 215, 251, 260, 261, 424, 427, 434, 456, 472, 473, 476, 479, 481, 489, 491, 493, 495, 503, 505, 509, 515, 516, 518, 519, 520, 522, 537, 541, 542, 546, 553, 561, 563, 564, 570, 576, 577, 578, 581, 582, 584, 585, 586, 589, 592, 599, 601, 617, 621, 623, 639, 654

In this test for example, the following compounds from the preparation examples showed good activity of 90% at an application rate of 20 ppm: 56, 227, 250, 276, 323, 430, 432, 460, 549, 558, 560, 575 In this test for example, the following compounds from the preparation examples showed good activity of 80% at an application rate of 20 ppm: 216, 235, 253, 273, 426, 440, 494, 502, 513, 528, 551, 559

In this test for example, the following compounds from the preparation examples showed good activity of 90% at an application rate of 4 ppm: 451

In this test for example, the following compounds from the preparation examples showed good activity of 80% at an application rate of 4 ppm: 579

Haemonchus contortus—Test (HAEMCO)
Solvent: dimethyl sulfoxide

To produce a suitable preparation of active compound, 10 mg of active compound are dissolved in 0.5 ml solvent, and the concentrate is diluted with “Ringer's solution” to the desired concentration.

Approximately 40 larvae of the red stomach worm (Haemonchus contortus) are transferred into a test tube containing compound solution.

After 5 days percentage of larval mortality are recorded. 100% efficacy means all larvae are killed, 0% efficacy means no larvae are killed.

In this test for example, the following compounds from the preparation examples showed good activity of 100% at an application rate of 20 ppm: 215, 216, 260, 261, 424, 427, 432, 434, 456, 460, 472, 473, 476, 479, 481, 489, 491, 493, 495, 496, 501, 503, 509, 515, 516, 518, 519, 520, 522, 540, 541, 542, 546, 553, 558, 561, 563, 570, 578, 579, 580, 581, 582, 584, 585, 586, 589, 599, 617, 623

In this test for example, the following compounds from the preparation examples showed good activity of 90% at an application rate of 20 ppm: 273, 276, 425, 494, 513, 528, 537, 544, 560, 572, 575, 577, 598, 601, 621, 654

In this test for example, the following compounds from the preparation examples showed good activity of 80% at an application rate of 20 ppm: 56, 203, 227, 274, 323, 337, 430, 438, 440, 465, 478, 502, 505, 538, 543, 551, 576, 592, 639

Meioidovnve incomita—Test (MELGIN)

Solvent: 125.0 parts by weight of acetone

To produce a suitable preparation of active compound, 1 part by weight of active compound is mixed with the stated amount of solvent, and the concentrate is diluted with water to the desired concentration.

Vessels are filled with sand, a solution of the active ingredient, a suspension containing eggs and larvae of the southern root-knot nematode (Meloidogyne incognita) and salad seeds. The salad seeds germinate and the seedlings grow. Galls develop in the roots.

After 14 days the nematicidal activity is determined on the basis of the percentage of gall formation. 100% means that no galls were found; 0% means the number of galls found on the roots of the treated plants was equal to that in untreated control plants.

In this test, for example, the following compounds from the preparation examples showed good activity of 100% at an application rate of 20 ppm: 212, 213, 214, 216, 227, 235, 236, 247, 250, 251, 252, 253, 260, 261, 297, 313, 346, 352, 353, 358, 414, 424, 425, 426, 427, 430, 432, 456, 461, 462, 465, 479, 481, 489, 491, 493, 495, 501, 503, 509, 513, 515, 516, 517, 518, 520, 528, 532, 537, 538, 540, 541, 542, 545, 548, 549, 550, 551, 553, 558, 559, 560, 561, 563, 564, 570, 571, 572, 574, 575, 576, 577, 584, 585, 586, 589, 592, 595, 599, 601, 608, 609, 617, 621, 623, 630, 638, 639, 641, 645, 647, 649, 684, 695

In this test, for example, the following compounds from the preparation examples showed good activity of 90% at an application rate of 20 ppm: 11, 82, 86, 115, 215, 217, 225, 248, 254, 256, 271, 296, 311, 322, 348, 349, 434, 440, 443, 472, 473, 476, 482, 485, 487, 494, 505, 519, 522, 523, 543, 544, 555, 556, 566, 567, 587, 594, 611, 619, 631, 642

1. A compound of formula (I) 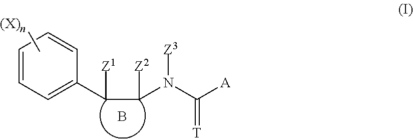
wherein
A represents phenyl of formula A^{a} 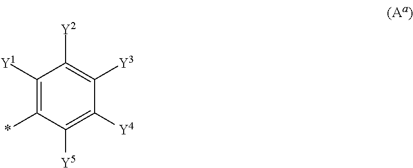
wherein
* indicates the bond which connects A^{a} to the C=T moiety of the compound of formula (I),
Y¹ represents halogen, nitro, SH, SF₅, CHO, OCHO, NHCHO, cyano, C₁-C₈-alkyl, C₁-C₈-halogenoalkyl having 1 to 5 halogen atoms, C₂-C₈-alkenyl, C₂-C₈-alkynyl, C₃-C₆-cycloalkyl, C₃-C₆-halogenocycloalkyl having 1 to 9 halogen atoms, C₁-C₈-alkylsulfanyl, —C₁-C₈-halogenoalkylsulfanyl having 1 to 5 halogen atoms, C₁-C₈-alkoxy, C₁-C₈-halogenoalkoxy having 1 to 5 halogen atoms, C₁-C₈-alkoxy-C₁-C₈-alkyl, C₂-C₈-alkenyloxy-C₁-C₈-alkyl, C₃-C₈-alkynyloxy-C₁-C₈-alkyl, C₂-C₈-alkenyloxy, C₃-C₈-alkynyloxy, C₁-C₈-alkoxycarbonyl, C₁-C₈-halogenoalkoxycarbonyl having 1 to 5 halogen atoms, C₁-C₈-alkylcarbonyloxy, C₁-C₈-halogenoalkylcarbonyloxy having 1 to 5 halogen atoms, C₁-C₈-alkylsulfinyl, C₁-C₈-halogenoalkylsulfinyl having 1 to 5 halogen atoms, C₁-C₈-alkylsulfonyl, C₁-C₈-halogenoalkylsulfonyl having 1 to 5 halogen atoms, C₁-C₈-alkylsulfonamide, substituted or unsubstituted tri-(C₁-C₈)-alkylsilyl, substituted or unsubstituted aryl and substituted or unsubstituted aryloxy;
Y², Y³, Y⁴ and Y⁵ independently from each other represent hydrogen or Y¹; or
A represents a carbo-linked heterocyclyl group optionally substituted by 1 to five groups R, wherein
R independently from each other R represents hydrogen, halogen, nitro, cyano, hydroxy, amino, sulfanyl, pentafluoro-λ⁶-sulfanyl, substituted or unsubstituted (C₁-C₈-alkoxyimino)-C₁-C₈-alkyl, substituted or unsubstituted (benzyloxyimino)-C₁-C₈-alkyl, substituted or unsubstituted C₁-C₈-alkyl, C₁-C₈-halogenoalkyl having 1 to 5 halogen atoms, substituted or unsubstituted C₂-C₈-alkenyl, C₂-C₈-halogenoalkenyl having 1 to 5 halogen atoms, substituted or unsubstituted C₂-C₈-alkynyl, C₂-C₈-halogenoalkynyl having 1 to 5 halogen atoms, substituted or unsubstituted C₁-C₈-alkoxy, C₁-C₈-halogenoalkoxy having 1 to 5 halogen atoms, substituted or unsubstituted C₁-C₈-alkylsulfanyl, C₁-C₈-halogenoalkylsulfanyl having 1 to 5 halogen atoms, substituted or unsubstituted C₁-C₈-alkylsulfinyl, C₁-C₈-halogenoalkylsulfinyl having 1 to 5 halogen atoms, substituted or unsubstituted C₁-C₈-alkylsulfonyl, C₁-C₈-halogenoalkylsulfonyl having 1 to 5 halogen atoms, substituted or unsubstituted C₁-C₈-alkylamino, substituted or unsubstituted di-C₁-C₈-alkylamino, substituted or unsubstituted C₂-C₈-alkenyloxy, substituted or unsubstituted C₃-C₈-alkynyloxy, substituted or unsubstituted C₃-C₇-cycloalkyl, C₃-C₇-halogenocycloalkyl having 1 to 5 halogen atoms, substituted or unsubstituted tri-(C₁-C₈-alkyl)-silyl, substituted or unsubstituted C₁-C₈-alkylcarbonyl, C₁-C₈-halogenoalkylcarbonyl having 1 to 5 halogen atoms, substituted or unsubstituted C₁-C₈-alkoxycarbonyl, C₁-C₈-halogenoalkoxycarbonyl having 1 to 5 halogen atoms, substituted or unsubstituted C₁-C₈-alkylcarbamoyl, substituted or unsubstituted di-(C₁-C₈)-alkylcarbamoyl, phenoxy, phenylsulfanyl, phenylamino, benzyloxy, benzylsulfanyl, or benzylamino,
T represents oxygen or sulphur,
B represents a 3-, 4-, 5-, 6- or 7-membered non-aromatic carbocycle optionally substituted by substituents selected from the group consisting of one to four C₁-C₈-alkyl groups and one to four halogen atoms,
n represents 0, 1, 2, 3, 4 or 5,
X independently from each other X represents halogen, nitro, cyano, isonitrile, hydroxy, amino, sulfanyl, pentafluoro-λ⁶-sulfanyl, formyl, formyloxy, formylamino, substituted or unsubstituted (hydroxyimino)-C₁-C₈-alkyl, substituted or unsubstituted (C₁-C₈-alkoxyimino)-C₁-C₈-alkyl, substituted or unsubstituted (C₂-C₈-alkenyloxyimino)-C₁-C₈-alkyl, substituted or unsubstituted (C₃-C₈-alkynyloxyimino)-C₁-C₈-alkyl, substituted or unsubstituted (benzyloxyimino)-C₁-C₈-alkyl, carboxy, carbamoyl, thiocarbamoyl, N-hydroxycarbamoyl, carbamate, substituted or unsubstituted C₁-C₈-alkyl, C₁-C₈-halogenoalkyl having 1 to 9 halogen atoms, substituted or unsubstituted C₂-C₈-alkenyl, C₂-C₈-halogenoalkenyl having 1 to 9 halogen atoms, substituted or unsubstituted C₂-C₈-alkynyl, C₂-C₈-halogenoalkynyl having 1 to 9 halogen atoms, substituted or unsubstituted C₁-C₈-alkoxy, C₁-C₈-halogenoalkoxy having 1 to 9 halogen atoms, substituted or unsubstituted C₁-C₈-alkylsulfanyl, C₁-C₈-halogenoalkylsulfanyl having 1 to 9 halogen atoms, substituted or unsubstituted C₁-C₈-alkylsulfinyl, C₁-C₈-halogenoalkylsulfinyl having 1 to 9 halogen atoms, substituted or unsubstituted C₁-C₈-alkylsulfonyl, C₁-C₈-halogenoalkylsulfonyl having 1 to 9 halogen atoms, substituted or unsubstituted C₁-C₈-alkylamino, substituted or unsubstituted di-(C₁-C₈-alkyl)-amino, substituted or unsubstituted C₂-C₈-alkenyloxy, C₂-C₈-halogenoalkenyloxy having 1 to 9 halogen atoms, substituted or unsubstituted C₃-C₈-alkynyloxy, C₃-C₈-halogenoalkynyloxy having 1 to 9 halogen atoms, substituted or unsubstituted C₃-C₇-cycloalkyl, C₃-C₇-halogenocycloalkyl having 1 to 9 halogen atoms, substituted or unsubstituted C₃-C₇-cycloalkyloxy, substituted or unsubstituted C₄-C₇-cycloalkenyl, C₄-C₇-halogenocycloalkenyl having 1 to 9 halogen atoms, substituted or unsubstituted (C₃-C₇-cycloalkyl)-C₁-C₈-alkyl, substituted or unsubstituted (C₃-C₇-cycloalkyl)-C₂-C₈-alkenyl, substituted or unsubstituted (C₃-C₇-cycloalkyl)-C₂-C₈-alkynyl, substituted or unsubstituted tri-(C₁-C₈-alkyl)-silyl, substituted or unsubstituted tri-(C₁-C₈-alkyl)-silyl-C₁-C₈-alkyl, substituted or unsubstituted C₁-C₈-alkylcarbonyl, C₁-C₈-halogenoalkylcarbonyl having 1 to 9 halogen atoms, substituted or unsubstituted C₁-C₈-alkylcarbonyloxy, C₁-C₈-halogenoalkylcarbonyloxy having 1 to 9 halogen atoms, substituted or unsubstituted C₁-C₈-alkylcarbonylamino, C₁-C₈-halogenoalkylcarbonylamino having 1 to 9 halogen atoms, substituted or unsubstituted C₁-C₈-alkoxycarbonyl, C₁-C₈-halogenoalkoxycarbonyl having 1 to 9 halogen atoms, substituted or unsubstituted C₁-C₈-alkyloxycarbonyloxy, C₁-C₈-halogenoalkoxycarbonyloxy having 1 to 9 halogen atoms, substituted or unsubstituted C₁-C₈-alkylcarbamoyl, substituted or unsubstituted di-C₁-C₈-alkylcarbamoyl, substituted or unsubstituted C₁-C₈-alkylaminocarbonyloxy, substituted or unsubstituted di-C₁-C₈-alkylaminocarbonyloxy, substituted or unsubstituted N—(C₁-C₈-alkyl)-hydroxycarbamoyl, substituted or unsubstituted C₁-C₈-alkoxycarbamoyl, substituted or unsubstituted N—(C₁-C₈-alkyl)-C₁-C₈-alkoxycarbamoyl, aryl optionally substituted by 1 to 6 groups Q which can be the same or different, aryl-C₁-C₈-alkyl optionally substituted by 1 to 6 groups Q which can be the same or different, aryl-C₂-C₈-alkenyl optionally substituted by 1 to 6 groups Q which can be the same or different, aryl-C₂-C₈-alkynyl optionally substituted by 1 to 6 groups Q which can be the same or different, aryloxy optionally substituted by 1 to 6 groups Q which can be the same or different, arylsulfanyl optionally substituted by 1 to 6 groups Q which can be the same or different, arylamino optionally substituted by 1 to 6 groups Q which can be the same or different, aryl-C₁-C₈-alkyloxy optionally substituted by 1 to 6 groups Q which can be the same or different, aryl-C₁-C₈-alkylsulfanyl optionally substituted by 1 to 6 groups Q which can be the same or different, aryl-C₁-C₈-alkylamino optionally substituted by 1 to 6 groups Q which can be the same or different, pyridinyl which can be substituted by 1 to 4 groups Q, pyridinyloxy which is optionally substituted by 1 to 4 groups Q, or
two substituents X together with the carbon atoms to which they are attached form a 5- or 6-membered, saturated carbocycle or saturated heterocycle, which is optionally substituted by 1 to 4 groups Q which can be the same or different,
Z¹ and Z² independently represent hydrogen, halogen, cyano, substituted or unsubstituted C₁-C₈-alkyl, C₁-C₈-halogenoalkyl having 1 to 5 halogen atoms, substituted or unsubstituted C₁-C₈-alkoxy, substituted or unsubstituted C₁-C₈-alkylsulfanyl, or substituted or unsubstituted C₁-C₈-alkoxycarbonyl,
Z³ represents hydrogen, cyano, C₁-C₈-alkoxy, unsubstituted C₃-C₇-cycloalkyl or C₃-C₇-cycloalkyl substituted by 1 to 10 substituents that can be the same or different, selected from the list consisting of halogen, cyano, C₁-C₈-alkyl, C₁-C₈-halogenoalkyl comprising 1 to 9 halogen atoms, C₁-C₈-alkoxy, C₁-C₈-halogenoalkoxy comprising 1 to 9 halogen atoms, C₁-C₈-alkoxycarbonyl, C₁-C₈-halogenoalkoxycarbonyl comprising 1 to 9 halogen atoms, C₁-C₈-alkylaminocarbonyl and di-(C₁-C₈-alkyl)-amino,
Q represents halogen, cyano, nitro, substituted or unsubstituted C₁-C₈-alkyl, C₁-C₈-halogenoalkyl having 1 to 9 halogen atoms, substituted or unsubstituted C₁-C₈-alkoxy, C₁-C₈-halogenoalkoxy having 1 to 9 halogen atoms, substituted or unsubstituted C₁-C₈-alkylsulfanyl, C₁-C₈-halogenoalkylsulfanyl having 1 to 9 halogen atoms, substituted or unsubstituted tri-(C₁-C₈-alkyl)-silyl, substituted or unsubstituted tri-(C₁-C₈-alkyl)-silyl-C₁-C₈-alkyl, substituted or unsubstituted C₁-C₈-alkoxyimino-C₁-C₈-alkyl, substituted or unsubstituted (benzyloxyimino)-C₁-C₈-alkyl,
And/or an N-oxide, metallic complex, metalloidic complex and/or an optically active isomer of a compound of formula (I),
capable of being used for controlling nematodes.

2. A compound according to claim 1 in which
A is selected from the group consisting of 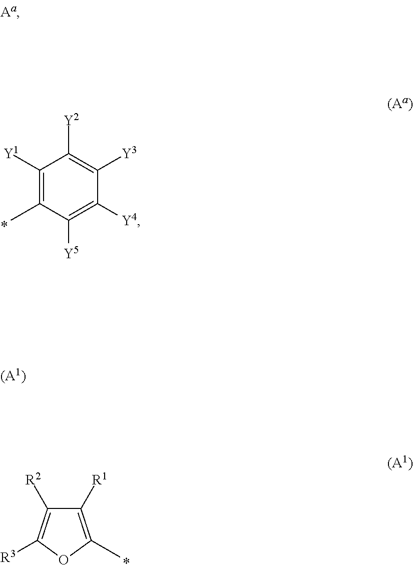
wherein
R¹ to R³ independently from each other represent hydrogen, halogen, substituted or unsubstituted C₁-C₅-alkyl, C₁-C₅-halogenoalkyl comprising 1 to 9 halogen atoms, substituted or unsubstituted C₁-C₅-alkoxy or C₁-C₅-halogenoalkoxy comprising 1 to 9 halogen atoms, 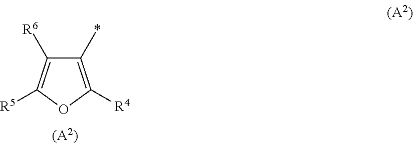
wherein
R⁴ to R⁶ independently from each other represent hydrogen, halogen, substituted or unsubstituted C₁-C₅-alkyl, C₁-C₅-halogenoalkyl comprising 1 to 9 halogen atoms, substituted or unsubstituted C₁-C₅-alkoxy or C₁-C₅-halogenoalkoxy comprising 1 to 9 halogen atoms, 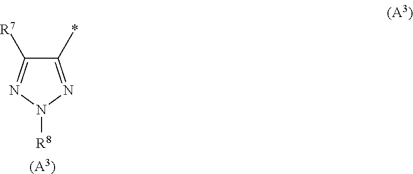
wherein
R⁷ represents hydrogen, halogen, substituted or unsubstituted C₁-C₅-alkyl, C₁-C₅-halogenoalkyl comprising 1 to 9 halogen atoms, substituted or unsubstituted C₁-C₅-alkoxy or C₁-C₅-halogenoalkoxy comprising 1 to 9 halogen atoms, and
R⁸ represents hydrogen or substituted or unsubstituted C₁-C₅-alkyl, 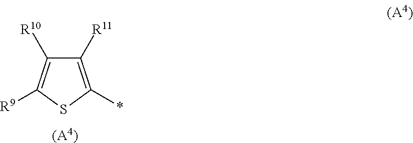
wherein
R⁹ to R¹¹ independently from each other represent hydrogen, halogen, substituted or unsubstituted C₁-C₅-alkyl, amino, substituted or unsubstituted C₁-C₅-alkoxy, substituted or unsubstituted C₁-C₅-alkylsulphanyl, C₁-C₅-halogenoalkyl comprising 1 to 9 halogen atoms or C₁-C₅-halogenoalkoxy comprising 1 to 9 halogen atoms, 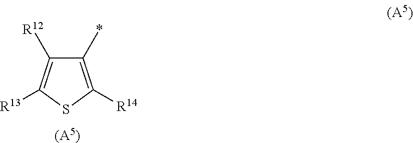
wherein
R¹² and R¹³ independently from each other represent hydrogen, halogen, substituted or unsubstituted C₁-C₅-alkyl, substituted or unsubstituted C₁-C₅-alkoxy, amino, C₁-C₅-halogenoalkyl comprising 1 to 9 halogen atoms or C₁-C₅-halogenoalkoxy comprising 1 to 9 halogen atoms, and
R¹⁴ represents hydrogen, halogen, substituted or unsubstituted C₁-C₅-alkyl, substituted or unsubstituted C₁-C₅-alkoxy, amino, C₁-C₅-halogenoalkyl comprising 1 to 9 halogen atoms or C₁-C₅-halogenoalkoxy comprising 1 to 9 halogen atoms, 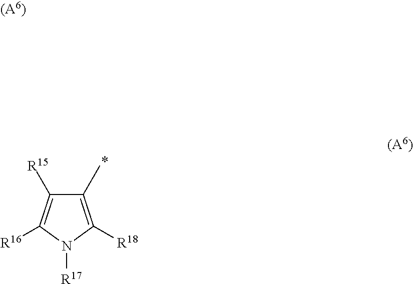
wherein
R¹⁵ represents hydrogen, halogen, cyano, substituted or unsubstituted C₁-C₅-alkyl, substituted or unsubstituted C₁-C₅-alkoxy, C₁-C₅-halogenoalkoxy comprising 1 to 9 halogen atoms or C₁-C₅-halogenoalkyl comprising 1 to 9 halogen atoms, and
R¹⁶ and R¹⁸ independently from each other represent hydrogen, halogen, substituted or unsubstituted C₁-C₅-alkoxycarbonyl, substituted or unsubstituted C₁-C₅-alkyl, C₁-C₅-halogenoalkoxy comprising 1 to 9 halogen atoms or C₁-C₅-halogenoalkyl comprising 1 to 9 halogen atoms, and
R¹⁷ represents hydrogen or substituted or unsubstituted C₁-C₅-alkyl, 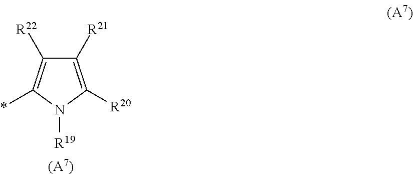
wherein
R¹⁹ represents hydrogen or substituted or unsubstituted C₁-C₅-alkyl, and
R²⁰ to R²² independently from each other represent hydrogen, halogen, substituted or unsubstituted C₁-C₅-alkyl or C₁-C₅-halogenoalkyl comprising 1 to 9 halogen atoms, 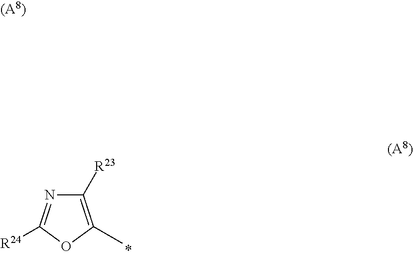
wherein
R²³ represents hydrogen, halogen, substituted or unsubstituted C₁-C₅-alkyl or C₁-C₅-halogenoalkyl comprising 1 to 9 halogen atoms, and
R²⁴ represents hydrogen or substituted or unsubstituted C₁-C₅-alkyl or C₁-C₅-halogenoalkyl comprising 1 to 9 halogen atoms, 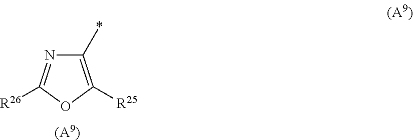
wherein
R²⁵ represents hydrogen, halogen, substituted or unsubstituted C₁-C₅-alkyl or C₁-C₅-halogenoalkyl comprising 1 to 9 halogen atoms, and
R²⁶ represents hydrogen, substituted or unsubstituted C₁-C₅-alkyl or C₁-C₅-halogenoalkyl comprising 1 to 9 halogen atoms, 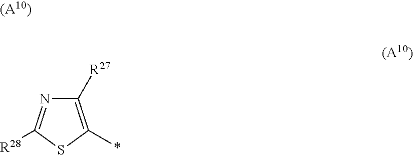
wherein
R²⁷ represents hydrogen, halogen, substituted or unsubstituted C₁-C₅-alkyl or C₁-C₅-halogenoalkyl comprising 1 to 9 halogen atoms, and
R²⁸ represents hydrogen, halogen, substituted or unsubstituted C₁-C₅-alkyl, C₁-C₅-halogenoalkyl comprising 1 to 9 halogen atoms, C₁-C₅-halogenoalkoxy comprising 1 to 9 halogen atoms, amino, substituted or unsubstituted C₁-C₅-alkylamino or substituted or unsubstituted di-(C₁-C₅-alkyl)-amino, 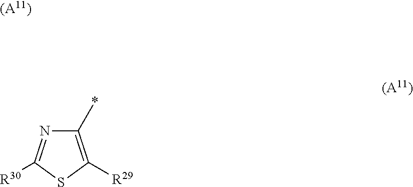
wherein
R²⁹ represents hydrogen, halogen, substituted or unsubstituted C₁-C₅-alkyl, substituted or unsubstituted C₁-C₅-alkoxy, C₁-C₅-halogenoalkoxy comprising 1 to 9 halogen atoms or C₁-C₅-halogenoalkyl comprising 1 to 9 halogen atoms, and
R³⁰ represents hydrogen, halogen, substituted or unsubstituted C₁-C₅-alkyl, C₁-C₅-halogenoalkyl comprising 1 to 9 halogen atoms, C₁-C₅-halogenoalkoxy comprising 1 to 9 halogen atoms, amino, substituted or unsubstituted C₁-C₅-alkylamino or substituted or unsubstituted di-(C₁-C₅-alkyl)-amino, 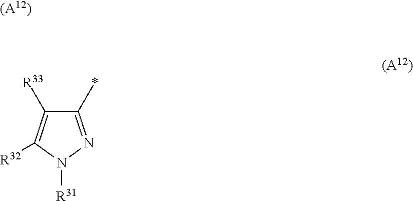
wherein
R³¹ represents hydrogen or substituted or unsubstituted C₁-C₅-alkyl, and
R³² represents hydrogen, halogen, substituted or unsubstituted C₁-C₅-alkyl or C₁-C₅-halogenoalkyl comprising 1 to 9 halogen atoms, and
R³³ represents hydrogen, halogen, nitro, substituted or unsubstituted C₁-C₅-alkyl, substituted or unsubstituted C₁-C₅-alkoxy, C₁-C₅-halogenoalkoxy comprising 1 to 9 halogen atoms or C₁-C₅-halogenoalkyl comprising 1 to 9 halogen atoms, 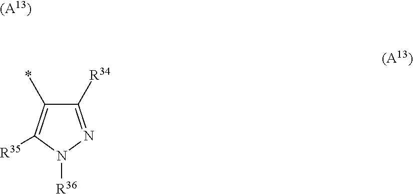
wherein
R³⁴ represents hydrogen, halogen, substituted or unsubstituted C₁-C₅-alkyl, substituted or unsubstituted C₃-C₅-cycloalkyl, C₁-C₅-halogenoalkyl comprising 1 to 9 halogen atoms, substituted or unsubstituted C₁-C₅-alkoxy, substituted or unsubstituted C₃-C₅-alkynyloxy or C₁-C₅-halogenoalkoxy comprising 1 to 9 halogen atoms, and
R³⁵ represents hydrogen, halogen, substituted or unsubstituted C₁-C₅-alkyl, cyano, substituted or unsubstituted C₁-C₅-alkoxy, substituted or unsubstituted C₁-C₅-alkylsulphanyl, C₁-C₅-halogenoalkyl comprising 1 to 9 halogen atoms, C₁-C₅-halogenoalkoxy comprising 1 to 9 halogen atoms, amino, substituted or unsubstituted C₁-C₅-alkylamino or substituted or unsubstituted di-(C₁-C₅-alkyl)-amino, and
R³⁶ represents a hydrogen atom or substituted or unsubstituted C₁-C₅-alkyl, 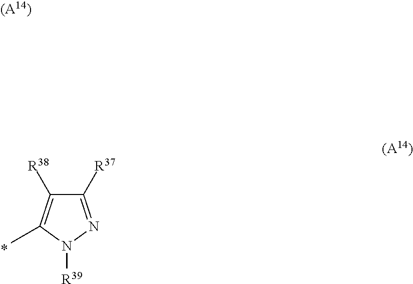
wherein
R³⁷ and R³⁸ independently from each other represent hydrogen, halogen, substituted or unsubstituted C₁-C₅-alkyl, C₁-C₅-halogenoalkyl comprising 1 to 9 halogen atoms, substituted or unsubstituted C₁-C₅-alkoxy or a substituted or unsubstituted C₁-C₅-alkylsulphanyl, and
R³⁹ represents hydrogen or substituted or unsubstituted C₁-C₅-alkyl, 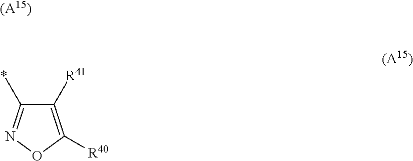
wherein
R⁴⁰ and R⁴¹ independently from each other represent hydrogen, halogen, substituted or unsubstituted C₁-C₅-alkyl or C₁-C₅-halogenoalkyl comprising 1 to 9 halogen atoms, 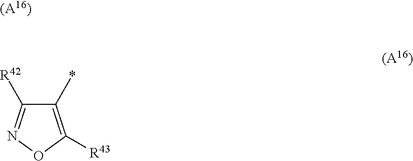
wherein
R⁴² and R⁴³ independently from each other represent hydrogen, halogen, substituted or unsubstituted C₁-C₅-alkyl, C₁-C₅-halogenoalkyl comprising 1 to 9 halogen atoms or amino, 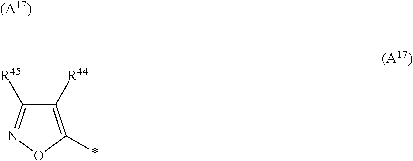
wherein
R⁴⁴ and R⁴⁵ independently from each other represent hydrogen, halogen, substituted or unsubstituted C₁-C₅-alkyl or C₁-C₅-halogenoalkyl comprising 1 to 9 halogen atoms, 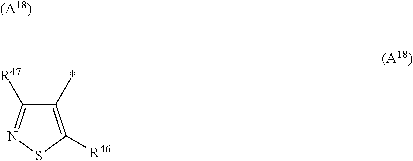
wherein
R⁴⁷ represents hydrogen, halogen, substituted or unsubstituted C₁-C₅-alkyl or C₁-C₅-halogenoalkyl comprising 1 to 9 halogen atoms, and
R⁴⁶ represents hydrogen, halogen, substituted or unsubstituted C₁-C₅-alkyl, C₁-C₅-halogenoalkyl comprising 1 to 9 halogen atoms or substituted or unsubstituted C₁-C₅-alkylsulfanyl, 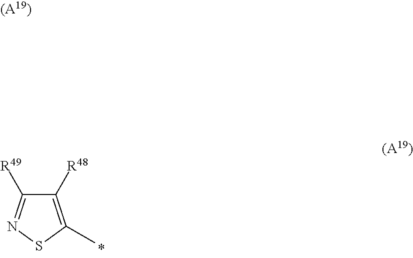
wherein
R⁴⁹ and R⁴⁸ independently from each other represent hydrogen, halogen, substituted or unsubstituted C₁-C₅-alkyl, substituted or unsubstituted C₁-C₅-alkoxy, C₁-C₅-halogenoalkoxy comprising 1 to 9 halogen atoms or C₁-C₅-halogenoalkyl comprising 1 to 9 halogen atoms, 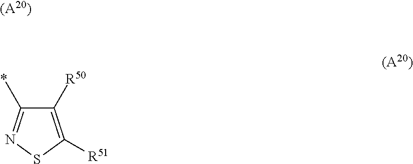
wherein
R⁵⁰ and R⁵¹ independently from each other represent hydrogen, halogen, substituted or unsubstituted C₁-C₅-alkyl, substituted or unsubstituted C₁-C₅-alkoxy, C₁-C₅-halogenoalkoxy comprising 1 to 9 halogen atoms or C₁-C₅-halogenoalkyl comprising 1 to 9 halogen atoms, 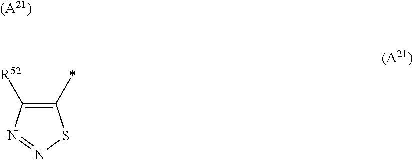
wherein
R⁵² represents hydrogen, halogen, substituted or unsubstituted C₁-C₅-alkyl or C₁-C₅-halogenoalkyl comprising 1 to 9 halogen atoms, 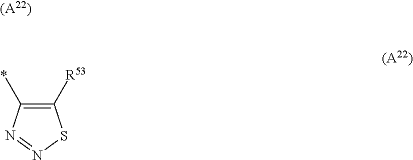
wherein
R⁵³ represents hydrogen, halogen, substituted or unsubstituted C₁-C₅-alkyl or C₁-C₅-halogenoalkyl comprising 1 to 9 halogen atoms, 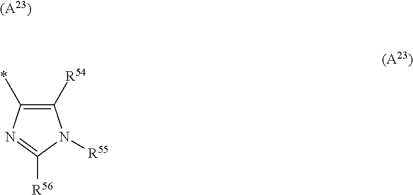
wherein
R⁵⁴ and R⁵⁶ independently from each other represent hydrogen, halogen, substituted or unsubstituted C₁-C₅-alkyl or C₁-C₅-halogenoalkyl comprising 1 to 9 halogen atoms, and
R⁵⁵ represents hydrogen or substituted or unsubstituted C₁-C₅-alkyl, 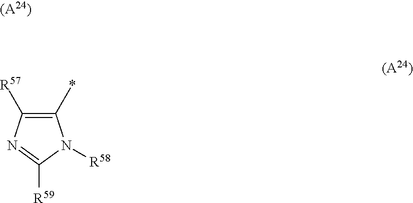
wherein
R⁵⁷ and R⁵⁹ independently from each other represent hydrogen, halogen, substituted or unsubstituted C₁-C₅-alkyl or C₁-C₅-halogenoalkyl comprising 1 to 9 halogen atoms, and
R⁵⁸ represents hydrogen or substituted or unsubstituted C₁-C₅-alkyl, 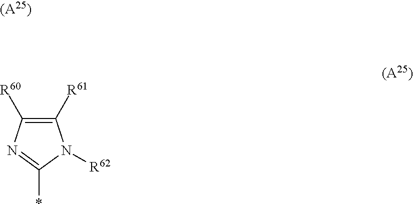
wherein
R⁶⁰ and R⁶¹ independently from each other represent hydrogen, halogen, substituted or unsubstituted C₁-C₅-alkyl or C₁-C₅-halogenoalkyl comprising 1 to 9 halogen atoms, and
R⁶² represents a hydrogen atom or substituted or unsubstituted C₁-C₅-alkyl, 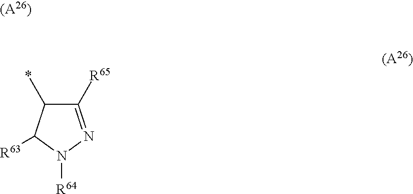
wherein
R⁶³ represents hydrogen, halogen, substituted or unsubstituted C₁-C₅-alkyl, cyano, substituted or unsubstituted C₁-C₅-alkoxy, substituted or unsubstituted C₁-C₅-alkylsulphanyl, C₁-C₅-halogenoalkyl comprising 1 to 9 halogen atoms, C₁-C₅-halogenoalkoxy comprising 1 to 9 halogen atoms, amino, substituted or unsubstituted C₁-C₅-alkylamino or di(C₁-C₅-alkyl)amino, and
R⁶⁴ represents hydrogen or substituted or unsubstituted C₁-C₅-alkyl, and
R⁶⁵ represents hydrogen, halogen, substituted or unsubstituted C₁-C₅-alkyl, substituted or unsubstituted C₃-C₅-cycloalkyl, C₁-C₅-halogenoalkyl comprising 1 to 9 halogen atoms, substituted or unsubstituted C₁-C₅-alkoxy, substituted or unsubstituted C₃-C₅-alkynyloxy or C₁-C₅-halogenoalkoxy comprising 1 to 9 halogen atoms, 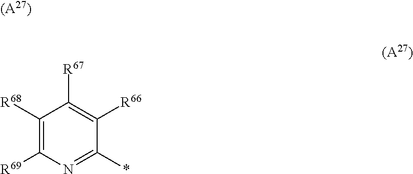
in which
R⁶⁶ represents hydrogen, halogen, hydroxy, cyano, C₁-C₄-alkyl, C₁-C₄-halogenoalkyl having 1 to 5 halogen atoms, C₁-C₄-alkoxy, C₁-C₄-alkylsulfanyl, C₁-C₄-halogenoalkylsulfanyl having 1 to 5 halogen atoms and C₁-C₄-halogenoalkoxy having 1 to 5 halogen atoms, and
R⁶⁷, R⁶⁸ and R⁶⁹ independently from each other represent hydrogen, halogen, cyano, C₁-C₄-alkyl, C₁-C₄-halogenoalkyl having 1 to 5 halogen atoms, C₁-C₄-alkoxy, C₁-C₄-alkylsulfanyl, C₁-C₄-halogenoalkoxy having 1 to 5 halogen atoms, SC₁-C₄-alkylsulfinyl and C₁-C₄-alkylsulfonyl, 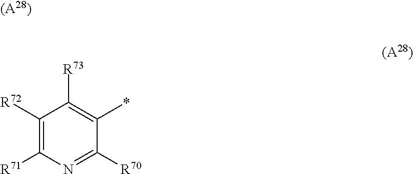
in which
R⁷⁰ represents hydrogen, halogen, hydroxy, cyano, C₁-C₄-alkyl, C₁-C₄-halogenoalkyl having 1 to 5 halogen atoms, C₁-C₄-alkoxy, C₁-C₅-alkylsulfanyl, C₂-C₅-alkenylsulfanyl, C₁-C₄-halogenoalkylsulfanyl having 1 to 5 halogen atoms, C₁-C₄-halogenoalkoxy having 1 to 5 halogen atoms, phenyloxy (optionally substituted by halogen or C₁-C₄-alkyl) and phenylsulfanyl (optionally substituted by halogen or C₁-C₄-alkyl), and
R⁷¹, R⁷² and R⁷³ independently from each other represent hydrogen, halogen, cyano, C₁-C₄-alkyl, C₁-C₄-halogenoalkyl having 1 to 5 halogen atoms, C₁-C₄-alkoxy, C₁-C₄-alkylsulfanyl, C₁-C₄-halogenoalkoxy having 1 to 5 halogen atoms, C₁-C₄-alkylsulfinyl, C₁-C₄-alkylsulfonyl, N-morpholine optionally substituted by halogen or C₁-C₄-alkyl, and thienyl (optionally substituted by halogen or C₁-C₄-alkyl), 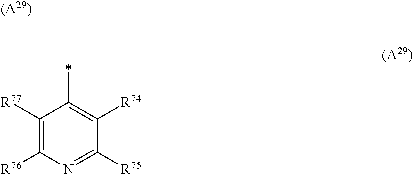
in which
R⁷⁴, R⁷⁵, R⁷⁶ and R⁷ independently from each other represent hydrogen, halogen, hydroxy, cyano, C₁-C₄-alkyl, C₁-C₄-halogenoalkyl having 1 to 5 halogen atoms, C₁-C₄-alkoxy, C₁-C₄-alkylsulfanyl, C₁-C₄-halogenoalkylsulfanyl having 1 to 5 halogen atoms, C₁-C₄-halogenoalkoxy having 1 to 5 halogen atoms, C₁-C₄-alkylsulfinyl and C₁-C₄-alkylsulfonyl, 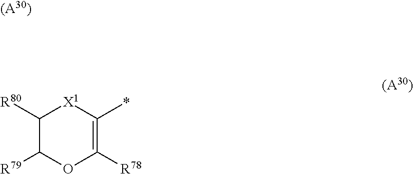
in which
X¹ represents —S—, —SO—, —SO₂— and —CH₂—, and
R⁷⁸ represents C₁-C₄-alkyl and C₁-C₄-halogenoalkyl having 1 to 5 halogen atoms, and
R⁷⁹ and R⁸⁰ independently from each other represent hydrogen and C₁-C₄-alkyl, 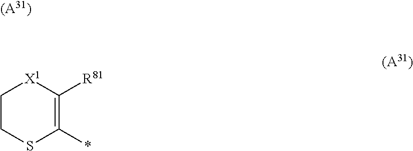
in which
R⁸¹ represents C₁-C₄-alkyl and C₁-C₄-halogenoalkyl having 1 to 5 halogen atoms, 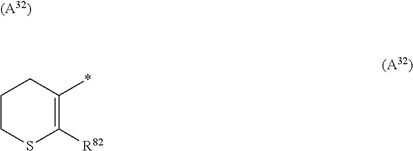
in which
R⁸² represents C₁-C₄-alkyl and C₁-C₄-halogenoalkyl having 1 to 5 halogen atoms and 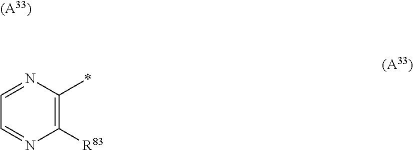
in which
R⁸³ represents hydrogen, halogen, C₁-C₄-alkyl and C₁-C₄-halogenoalkyl having 1 to 5 halogen atoms,
And/or an N-oxide, metallic complex, metalloidic complex and/or an optically active isomer thereof.

3. A compound according to claim 1, in which T represents sulphur, capable of being used for controlling nematodes.

4. A compound of formula (IB-1) 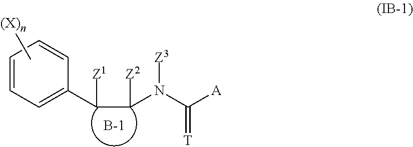
wherein
A represents phenyl of formula A^{a} 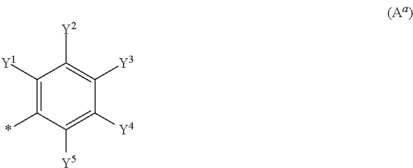
wherein
* indicates the bond which connects A^{a} to the C=T moiety of the compound of formula (IB-1),
Y¹ represents halogen, nitro, SH, SF₅, CHO, OCHO, NHCHO, cyano, C₁-C₈-alkyl, C₁-C₈-halogenoalkyl having 1 to 5 halogen atoms, C₂-C₈-alkenyl, C₂-C₈-alkynyl, C₃-C₆-cycloalkyl, C₃-C₆-halogenocycloalkyl having 1 to 9 halogen atoms, C₁-C₈-alkylsulfanyl, —C₁-C₈-halogenoalkylsulfanyl having 1 to 5 halogen atoms, C₁-C₈-alkoxy, C₁-C₈-halogenoalkoxy having 1 to 5 halogen atoms, C₁-C₈-alkoxy-C₁-C₈-alkyl, C₂-C₈-alkenyloxy-C₁-C₈-alkyl, C₃-C₈-alkynyloxy-C₁-C₈-alkyl, C₂-C₈-alkenyloxy, C₃-C₈-alkynyloxy, C₁-C₈-alkoxycarbonyl, C₁-C₈-halogenoalkoxycarbonyl having 1 to 5 halogen atoms, C₁-C₈-alkylcarbonyloxy, C₁-C₈-halogenoalkylcarbonyloxy having 1 to 5 halogen atoms, C₁-C₈-alkylsulfinyl, C₁-C₈-halogenoalkylsulfinyl having 1 to 5 halogen atoms, C₁-C₈-alkylsulfonyl, C₁-C₈-halogenoalkylsulfonyl having 1 to 5 halogen atoms, C₁-C₈-alkylsulfonamide, substituted or unsubstituted tri-(C₁-C₈)-alkylsilyl, substituted or unsubstituted aryl and substituted or unsubstituted aryloxy;
Y², Y³, Y⁴ and Y⁵ independently from each other represent hydrogen or Y¹; or
A represents a carbo-linked heterocyclyl group optionally substituted by 1 to five groups R, wherein
R independently from each other R represents hydrogen, halogen, nitro, cyano, hydroxy, amino, sulfanyl, pentafluoro-λ⁶-sulfanyl, substituted or unsubstituted (C₁-C₈-alkoxyimino)-C₁-C₈-alkyl, substituted or unsubstituted (benzyloxyimino)-C₁-C₈-alkyl, substituted or unsubstituted C₁-C₈-alkyl, C₁-C₈-halogenoalkyl having 1 to 5 halogen atoms, substituted or unsubstituted C₂-C₈-alkenyl, C₂-C₈-halogenoalkenyl having 1 to 5 halogen atoms, substituted or unsubstituted C₂-C₈-alkynyl, C₂-C₈-halogenoalkynyl having 1 to 5 halogen atoms, substituted or unsubstituted C₁-C₈-alkoxy, C₁-C₈-halogenoalkoxy having 1 to 5 halogen atoms, substituted or unsubstituted C₁-C₈-alkylsulfanyl, C₁-C₈-halogenoalkylsulfanyl having 1 to 5 halogen atoms, substituted or unsubstituted C₁-C₈-alkylsulfinyl, C₁-C₈-halogenoalkylsulfinyl having 1 to 5 halogen atoms, substituted or unsubstituted C₁-C₈-alkylsulfonyl, C₁-C₈-halogenoalkylsulfonyl having 1 to 5 halogen atoms, substituted or unsubstituted C₁-C₈-alkylamino, substituted or unsubstituted di-C₁-C₈-alkylamino, substituted or unsubstituted C₂-C₈-alkenyloxy, substituted or unsubstituted C₃-C₈-alkynyloxy, substituted or unsubstituted C₃-C₇-cycloalkyl, C₃-C₇-halogenocycloalkyl having 1 to 5 halogen atoms, substituted or unsubstituted tri-(C₁-C₈-alkyl)-silyl, substituted or unsubstituted C₁-C₈-alkylcarbonyl, C₁-C₈-halogenoalkylcarbonyl having 1 to 5 halogen atoms, substituted or unsubstituted C₁-C₈-alkoxycarbonyl, C₁-C₈-halogenoalkoxycarbonyl having 1 to 5 halogen atoms, substituted or unsubstituted C₁-C₈-alkylcarbamoyl, substituted or unsubstituted di-(C₁-C₈)-alkylcarbamoyl, phenoxy, phenylsulfanyl, phenylamino, benzyloxy, benzylsulfanyl, or benzylamino,
T represents oxygen or sulphur,
B-1 represents a 5-, 6- or 7-membered non-aromatic carbocycle optionally substituted by substituents selected from the group consisting of one to four C₁-C₈-alkyl groups and one to four halogen atoms,
n represents 0, 1, 2, 3, 4 or 5,
X independently from each other X represents halogen, nitro, cyano, isonitrile, hydroxy, amino, sulfanyl, pentafluoro-λ⁶-sulfanyl, formyl, formyloxy, formylamino, substituted or unsubstituted (hydroxyimino)-C₁-C₈-alkyl, substituted or unsubstituted (C₁-C₈-alkoxyimino)-C₁-C₈-alkyl, substituted or unsubstituted (C₂-C₈-alkenyloxyimino)-C₁-C₈-alkyl, substituted or unsubstituted (C₃-C₈-alkynyloxyimino)-C₁-C₈-alkyl, substituted or unsubstituted (benzyloxyimino)-C₁-C₈-alkyl, carboxy, carbamoyl, thiocarbamoyl, N-hydroxycarbamoyl, carbamate, substituted or unsubstituted C₁-C₈-alkyl, C₁-C₈-halogenoalkyl having 1 to 9 halogen atoms, substituted or unsubstituted C₂-C₈-alkenyl, C₂-C₈-halogenoalkenyl having 1 to 9 halogen atoms, substituted or unsubstituted C₂-C₈-alkynyl, C₂-C₈-halogenoalkynyl having 1 to 9 halogen atoms, substituted or unsubstituted C₁-C₈-alkoxy, C₁-C₈-halogenoalkoxy having 1 to 9 halogen atoms, substituted or unsubstituted C₁-C₈-alkylsulfanyl, C₁-C₈-halogenoalkylsulfanyl having 1 to 9 halogen atoms, substituted or unsubstituted C₁-C₈-alkylsulfinyl, C₁-C₈-halogenoalkylsulfinyl having 1 to 9 halogen atoms, substituted or unsubstituted C₁-C₈-alkylsulfonyl, C₁-C₈-halogenoalkylsulfonyl having 1 to 9 halogen atoms, substituted or unsubstituted C₁-C₈-alkylamino, substituted or unsubstituted di-(C₁-C₈-alkyl)-amino, substituted or unsubstituted C₂-C₈-alkenyloxy, C₂-C₈-halogenoalkenyloxy having 1 to 9 halogen atoms, substituted or unsubstituted C₃-C₈-alkynyloxy, C₃-C₈-halogenoalkynyloxy having 1 to 9 halogen atoms, substituted or unsubstituted C₃-C₇-cycloalkyl, C₃-C₇-halogenocycloalkyl having 1 to 9 halogen atoms, substituted or unsubstituted C₃-C₇-cycloalkyloxy, substituted or unsubstituted C₄-C₇-cycloalkenyl, C₄-C₇-halogenocycloalkenyl having 1 to 9 halogen atoms, substituted or unsubstituted (C₃-C₇-cycloalkyl)-C₁-C₈-alkyl, substituted or unsubstituted (C₃-C₇-cycloalkyl)-C₂-C₈-alkenyl, substituted or unsubstituted (C₃-C₇-cycloalkyl)-C₂-C₈-alkynyl, substituted or unsubstituted tri-(C₁-C₈-alkyl)-silyl, substituted or unsubstituted tri-(C₁-C₈-alkyl)-silyl-C₁-C₈-alkyl, substituted or unsubstituted C₁-C₈-alkylcarbonyl, C₁-C₈-halogenoalkylcarbonyl having 1 to 9 halogen atoms, substituted or unsubstituted C₁-C₈-alkylcarbonyloxy, C₁-C₈-halogenoalkylcarbonyloxy having 1 to 9 halogen atoms, substituted or unsubstituted C₁-C₈-alkylcarbonylamino, C₁-C₈-halogenoalkylcarbonylamino having 1 to 9 halogen atoms, substituted or unsubstituted C₁-C₈-alkoxycarbonyl, C₁-C₈-halogenoalkoxycarbonyl having 1 to 9 halogen atoms, substituted or unsubstituted C₁-C₈-alkyloxycarbonyloxy, C₁-C₈-halogenoalkoxycarbonyloxy having 1 to 9 halogen atoms, substituted or unsubstituted C₁-C₈-alkylcarbamoyl, substituted or unsubstituted di-C₁-C₈-alkylcarbamoyl, substituted or unsubstituted C₁-C₈-alkylaminocarbonyloxy, substituted or unsubstituted di-C₁-C₈-alkylaminocarbonyloxy, substituted or unsubstituted N—(C₁-C₈-alkyl)-hydroxycarbamoyl, substituted or unsubstituted C₁-C₈-alkoxycarbamoyl, substituted or unsubstituted N—(C₁-C₈-alkyl)-C₁-C₈-alkoxycarbamoyl, aryl optionally substituted by 1 to 6 groups Q which can be the same or different, aryl-C₁-C₈-alkyl optionally substituted by 1 to 6 groups Q which can be the same or different, aryl-C₂-C₈-alkenyl optionally substituted by 1 to 6 groups Q which can be the same or different, aryl-C₂-C₈-alkynyl optionally substituted by 1 to 6 groups Q which can be the same or different, aryloxy optionally substituted by 1 to 6 groups Q which can be the same or different, arylsulfanyl optionally substituted by 1 to 6 groups Q which can be the same or different, arylamino optionally substituted by 1 to 6 groups Q which can be the same or different, aryl-C₁-C₈-alkyloxy optionally substituted by 1 to 6 groups Q which can be the same or different, aryl-C₁-C₈-alkylsulfanyl optionally substituted by 1 to 6 groups Q which can be the same or different, aryl-C₁-C₈-alkylamino optionally substituted by 1 to 6 groups Q which can be the same or different, pyridinyl which can be substituted by 1 to 4 groups Q, pyridinyloxy which is optionally substituted by 1 to 4 groups Q, or
two substituents X together with the carbon atoms to which they are attached form a 5- or 6-membered, saturated carbocycle or saturated heterocycle, which is optionally substituted by 1 to 4 groups Q which can be the same or different,
Z¹ and Z² independently represent hydrogen, halogen, cyano, substituted or unsubstituted C₁-C₈-alkyl, C₁-C₈-halogenoalkyl having 1 to 5 halogen atoms, substituted or unsubstituted C₁-C₈-alkoxy, substituted or unsubstituted C₁-C₈-alkylsulfanyl, or substituted or unsubstituted C₁-C₈-alkoxycarbonyl,
Z³ represents hydrogen, cyano, C₁-C₈-alkoxy, unsubstituted C₃-C₇-cycloalkyl or C₃-C₇-cycloalkyl substituted by 1 to 10 substituents that can be the same or different, selected from the list consisting of halogen, cyano, C₁-C₈-alkyl, C₁-C₈-halogenoalkyl comprising 1 to 9 halogen atoms, C₁-C₈-alkoxy, C₁-C₈-halogenoalkoxy comprising 1 to 9 halogen atoms, C₁-C₈-alkoxycarbonyl, C₁-C₈-halogenoalkoxycarbonyl comprising 1 to 9 halogen atoms, C₁-C₈-alkylaminocarbonyl and di-(C₁-C₈-alkyl)-amino,
Q represents halogen, cyano, nitro, substituted or unsubstituted C₁-C₈-alkyl, C₁-C₈-halogenoalkyl having 1 to 9 halogen atoms, substituted or unsubstituted C₁-C₈-alkoxy, C₁-C₈-halogenoalkoxy having 1 to 9 halogen atoms, substituted or unsubstituted C₁-C₈-alkylsulfanyl, C₁-C₈-halogenoalkylsulfanyl having 1 to 9 halogen atoms, substituted or unsubstituted tri-(C₁-C₈-alkyl)-silyl, substituted or unsubstituted tri-(C₁-C₈-alkyl)-silyl-C₁-C₈-alkyl, substituted or unsubstituted C₁-C₈-alkoxyimino-C₁-C₈-alkyl, substituted or unsubstituted (benzyloxyimino)-C₁-C₈-alkyl,
And/or an N-oxide, metallic complex, metalloidic complex and/or an optically active isomer of a compound of formula (IB-1),
capable of being used for controlling nematodes.

5. Compound of formula (IB-1-1) 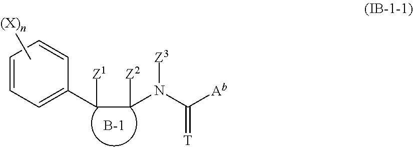
in which
Y¹ represents halogen, nitro, SH, SF₅, CHO, OCHO, NHCHO, cyano, C₁-C₈-alkyl, C₁-C₈-halogenoalkyl having 1 to 5 halogen atoms, C₂-C₈-alkenyl, C₂-C₈-alkynyl, C₃-C₆-cycloalkyl, C₃-C₆-halogenocycloalkyl having 1 to 9 halogen atoms, C₁-C₈-alkylsulfanyl, —C₁-C₈ halogenoalkylsulfanyl having 1 to 5 halogen atoms, C₁-C₈-alkoxy, C₁-C₈-halogenoalkoxy having 1 to 5 halogen atoms, C₁-C₈-alkoxy-C₁-C₈-alkyl, C₂-C₈-alkenyloxy-C₁-C₈-alkyl, C₃-C₈-alkynyloxy-C₁-C₈-alkyl, C₂-C₈-alkenyloxy, C₃-C₈-alkynyloxy, C₁-C₈-alkoxycarbonyl, C₁-C₈-halogenoalkoxycarbonyl having 1 to 5 halogen atoms, C₁-C₈-alkylcarbonyloxy, C₁-C₈-halogenoalkylcarbonyloxy having 1 to 5 halogen atoms, C₁-C₈-alkylsulfinyl, C₁-C₈-halogenoalkylsulfinyl having 1 to 5 halogen atoms, C₁-C₈-alkylsulfonyl, C₁-C₈-halogenoalkylsulfonyl having 1 to 5 halogen atoms, C₁-C₈-alkylsulfonamide, substituted or unsubstituted tri-(C₁-C₈)-alkylsilyl, substituted or unsubstituted aryl and substituted or unsubstituted aryloxy;
Y², Y³, Y⁴ and Y⁵ independently from each other represent hydrogen or Y¹; or
A represents a carbo-linked heterocyclyl group optionally substituted by 1 to five groups R, wherein
R independently from each other R represents hydrogen, halogen, nitro, cyano, hydroxy, amino, sulfanyl, pentafluoro-λ⁶-sulfanyl, substituted or unsubstituted (C₁-C₈-alkoxyimino)-C₁-C₈-alkyl, substituted or unsubstituted (benzyloxyimino)-C₁-C₈-alkyl, substituted or unsubstituted C₁-C₈-alkyl, C₁-C₈-halogenoalkyl having 1 to 5 halogen atoms, substituted or unsubstituted C₂-C₈-alkenyl, C₂-C₈-halogenoalkenyl having 1 to 5 halogen atoms, substituted or unsubstituted C₂-C₈-alkynyl, C₂-C₈-halogenoalkynyl having 1 to 5 halogen atoms, substituted or unsubstituted C₁-C₈-alkoxy, C₁-C₈-halogenoalkoxy having 1 to 5 halogen atoms, substituted or unsubstituted C₁-C₈-alkylsulfanyl, C₁-C₈-halogenoalkylsulfanyl having 1 to 5 halogen atoms, substituted or unsubstituted C₁-C₈-alkylsulfinyl, C₁-C₈-halogenoalkylsulfinyl having 1 to 5 halogen atoms, substituted or unsubstituted C₁-C₈-alkylsulfonyl, C₁-C₈-halogenoalkylsulfonyl having 1 to 5 halogen atoms, substituted or unsubstituted C₁-C₈-alkylamino, substituted or unsubstituted di-C₁-C₈-alkylamino, substituted or unsubstituted C₂-C₈-alkenyloxy, substituted or unsubstituted C₁-C₈-alkynyloxy, substituted or unsubstituted C₃-C₇-cycloalkyl, C₃-C₇-halogenocycloalkyl having 1 to 5 halogen atoms, substituted or unsubstituted tri-(C₁-C₈-alkyl)-silyl, substituted or unsubstituted C₁-C₈-alkylcarbonyl, C₁-C₈-halogenoalkylcarbonyl having 1 to 5 halogen atoms, substituted or unsubstituted C₁-C₈-alkoxycarbonyl, C₁-C₈-halogenoalkoxycarbonyl having 1 to 5 halogen atoms, substituted or unsubstituted C₁-C₈-alkylcarbamoyl, substituted or unsubstituted di-(C₁-C₈)-alkylcarbamoyl, phenoxy, phenylsulfanyl, phenylamino, benzyloxy, benzylsulfanyl, or benzylamino,
T represents oxygen or sulphur,
B-1 represents a 5-, 6- or 7-membered non-aromatic carbocycle optionally substituted by substituents selected from the group consisting of one to four C₁-C₈-alkyl groups and one to four halogen atoms,
n represents 0, 1, 2, 3, 4 or 5,
X independently from each other X represents halogen, nitro, cyano, isonitrile, hydroxy, amino, sulfanyl, pentafluoro-λ⁶-sulfanyl, formyl, formyloxy, formylamino, substituted or unsubstituted (hydroxyimino)-C₁-C₈-alkyl, substituted or unsubstituted (C₁-C₈-alkoxyimino)-C₁-C₈-alkyl, substituted or unsubstituted (C₂-C₈-alkenyloxyimino)-C₁-C₈-alkyl, substituted or unsubstituted (C₃-C₈-alkynyloxyimino)-C₁-C₈-alkyl, substituted or unsubstituted (benzyloxyimino)-C₁-C₈-alkyl, carboxy, carbamoyl, thiocarbamoyl, N-hydroxycarbamoyl, carbamate, substituted or unsubstituted C₁-C₈-alkyl, C₁-C₈-halogenoalkyl having 1 to 9 halogen atoms, substituted or unsubstituted C₂-C₈-alkenyl, C₂-C₈-halogenoalkenyl having 1 to 9 halogen atoms, substituted or unsubstituted C₂-C₈-alkynyl, C₂-C₈-halogenoalkynyl having 1 to 9 halogen atoms, substituted or unsubstituted C₁-C₈-alkoxy, C₁-C₈-halogenoalkoxy having 1 to 9 halogen atoms, substituted or unsubstituted C₁-C₈-alkylsulfanyl, C₁-C₈-halogenoalkylsulfanyl having 1 to 9 halogen atoms, substituted or unsubstituted C₁-C₈-alkylsulfinyl, C₁-C₈-halogenoalkylsulfinyl having 1 to 9 halogen atoms, substituted or unsubstituted C₁-C₈-alkylsulfonyl, C₁-C₈-halogenoalkylsulfonyl having 1 to 9 halogen atoms, substituted or unsubstituted C₁-C₈-alkylamino, substituted or unsubstituted di-(C₁-C₈-alkyl)-amino, substituted or unsubstituted C₂-C₈-alkenyloxy, C₂-C₈-halogenoalkenyloxy having 1 to 9 halogen atoms, substituted or unsubstituted C₃-C₈-alkynyloxy, C₃-C₈-halogenoalknyloxy having 1 to 9 halogen atoms, substituted or unsubstituted C₃-C₇-cycloalkyl, C₃-C₇-halogenocycloalkyl having 1 to 9 halogen atoms, substituted or unsubstituted C₃-C₇-cycloalkyloxy, substituted or unsubstituted C₄-C₇-cycloalkenyl, C₄-C₇-halogenocycloalkenyl having 1 to 9 halogen atoms, substituted or unsubstituted (C₃-C₇-cycloalkyl)-C₁-C₈-alkyl, substituted or unsubstituted (C₃-C₇-cycloalkyl)-C₂-C₈-alkenyl, substituted or unsubstituted (C₃-C₇-cycloalkyl)-C₂-C₈-alkynyl, substituted or unsubstituted tri-(C₁-C₈-alkyl)-silyl, substituted or unsubstituted tri-(C₁-C₈-alkyl)-silyl-C₁-C₈-alkyl, substituted or unsubstituted C₁-C₈-alkylcarbonyl, C₁-C₈-halogenoalkylcarbonyl having 1 to 9 halogen atoms, substituted or unsubstituted C₁-C₈-alkylcarbonyloxy, C₁-C₈-halogenoalkylcarbonyloxy having 1 to 9 halogen atoms, substituted or unsubstituted C₁-C₈-alkylcarbonylamino, C₁-C₈-halogenoalkylcarbonylamino having 1 to 9 halogen atoms, substituted or unsubstituted C₁-C₈-alkoxycarbonyl, C₁-C₈-halogenoalkoxycarbonyl having 1 to 9 halogen atoms, substituted or unsubstituted C₁-C₈-alkyloxycarbonyloxy, C₁-C₈-halogenoalkoxycarbonyloxy having 1 to 9 halogen atoms, substituted or unsubstituted C₁-C₈-alkylcarbamoyl, substituted or unsubstituted di-C₁-C₈-alkylcarbamoyl, substituted or unsubstituted C₁-C₈-alkylaminocarbonyloxy, substituted or unsubstituted di-C₁-C₈-alkylaminocarbonyloxy, substituted or unsubstituted N—(C₁-C₈-alkyl)-hydroxycarbamoyl, substituted or unsubstituted C₁-C₈-alkoxycarbamoyl, substituted or unsubstituted N—(C₁-C₈-alkyl)-C₁-C₈-alkoxycarbamoyl, aryl optionally substituted by 1 to 6 groups Q which can be the same or different, aryl-C₁-C₈-alkyl optionally substituted by 1 to 6 groups Q which can be the same or different, aryl-C₂-C₈-alkenyl optionally substituted by 1 to 6 groups Q which can be the same or different, aryl-C₁-C₈-alkynyl optionally substituted by 1 to 6 groups Q which can be the same or different, aryloxy optionally substituted by 1 to 6 groups Q which can be the same or different, arylsulfanyl optionally substituted by 1 to 6 groups Q which can be the same or different, arylamino optionally substituted by 1 to 6 groups Q which can be the same or different, aryl-C₁-C₈-alkyloxy optionally substituted by 1 to 6 groups Q which can be the same or different, aryl-C₁-C₈-alkylsulfanyl optionally substituted by 1 to 6 groups Q which can be the same or different, aryl-C₁-C₈-alkylamino optionally substituted by 1 to 6 groups Q which can be the same or different, pyridinyl which can be substituted by 1 to 4 groups Q, pyridinyloxy which is optionally substituted by 1 to 4 groups Q, or
two substituents X together with the carbon atoms to which they are attached form a 5- or 6-membered, saturated carbocycle or saturated heterocycle, which is optionally substituted by 1 to 4 groups Q which can be the same or different,
Z¹ and Z² independently represent hydrogen, halogen, cyano, substituted or unsubstituted C₁-C₈-alkyl, C₁-C₈-halogenoalkyl having 1 to 5 halogen atoms, substituted or unsubstituted C₁-C₈-alkoxy, substituted or unsubstituted C₁-C₈-alkylsulfanyl, or substituted or unsubstituted C₁-C₈-alkoxycarbonyl,
Q represents halogen, cyano, nitro, substituted or unsubstituted C₁-C₈-alkyl, C₁-C₈-halogenoalkyl having 1 to 9 halogen atoms, substituted or unsubstituted C₁-C₈-alkoxy, C₁-C₈-halogenoalkoxy having 1 to 9 halogen atoms, substituted or unsubstituted C₁-C₈-alkylsulfanyl, C₁-C₈-halogenoalkylsulfanyl having 1 to 9 halogen atoms, substituted or unsubstituted tri-(C₁-C₈-alkyl)-silyl, substituted or unsubstituted tri-(C₁-C₈-alkyl)-silyl-C₁-C₈-alkyl, substituted or unsubstituted C₁-C₈-alkoxyimino-C₁-C₈-alkyl, substituted or unsubstituted (benzyloxyimino)-C₁-C₈-alkyl,
Z³ represents hydrogen,
A^{b} represents A^{a} and represents A^{a} to A²⁶ 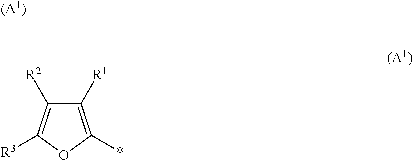
wherein
R¹ to R³ independently from each other represent hydrogen, halogen, substituted or unsubstituted C₁-C₅-alkyl, C₁-C₅-halogenoalkyl comprising 1 to 9 halogen atoms, substituted or unsubstituted C₁-C₅-alkoxy or C₁-C₅-halogenoalkoxy comprising 1 to 9 halogen atoms, 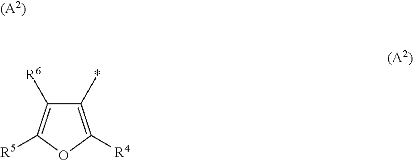
wherein
R⁴ to R⁶ independently from each other represent hydrogen, halogen, substituted or unsubstituted C₁-C₅-alkyl, C₁-C₅-halogenoalkyl comprising 1 to 9 halogen atoms, substituted or unsubstituted C₁-C₅-alkoxy or C₁-C₅-halogenoalkoxy comprising 1 to 9 halogen atoms, 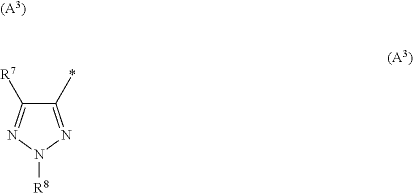
wherein
R⁷ represents hydrogen, halogen, substituted or unsubstituted C₁-C₅-alkyl, C₁-C₅-halogenoalkyl comprising 1 to 9 halogen atoms, substituted or unsubstituted C₁-C₅-alkoxy or C₁-C₅-halogenoalkoxy comprising 1 to 9 halogen atoms, and
R⁸ represents hydrogen or substituted or unsubstituted C₁-C₅-alkyl, 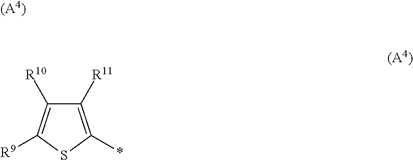
wherein
R⁹ to R¹¹ independently from each other represent hydrogen, halogen, substituted or unsubstituted C₁-C₅-alkyl, amino, substituted or unsubstituted C₁-C₅-alkoxy, substituted or unsubstituted C₁-C₅-alkylsulphanyl, C₁-C₅-halogenoalkyl comprising 1 to 9 halogen atoms or C₁-C₅-halogenoalkoxy comprising 1 to 9 halogen atoms, 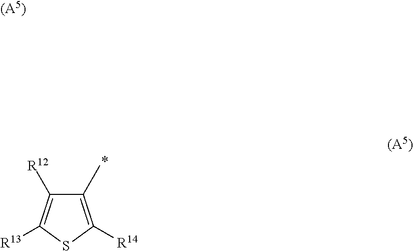
wherein
R¹² and R¹³ independently from each other represent hydrogen, halogen, substituted or unsubstituted C₁-C₅-alkyl, substituted or unsubstituted C₁-C₅-alkoxy, amino, C₁-C₅-halogenoalkyl comprising 1 to 9 halogen atoms or C₁-C₅-halogenoalkoxy comprising 1 to 9 halogen atoms, and
R¹⁴ represents hydrogen, halogen, substituted or unsubstituted C₁-C₅-alkyl, substituted or unsubstituted C₁-C₅-alkoxy, amino, C₁-C₅-halogenoalkyl comprising 1 to 9 halogen atoms or C₁-C₅-halogenoalkoxy comprising 1 to 9 halogen atoms, 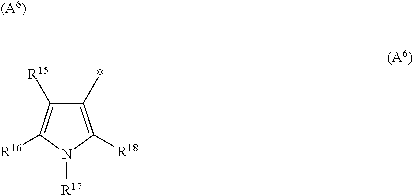
wherein
R¹⁵ represents hydrogen, halogen, cyano, substituted or unsubstituted C₁-C₅-alkyl, substituted or unsubstituted C₁-C₅-alkoxy, C₁-C₅-halogenoalkoxy comprising 1 to 9 halogen atoms or C₁-C₅-halogenoalkyl comprising 1 to 9 halogen atoms, and
R¹⁶ and R¹⁸ independently from each other represent hydrogen, halogen, substituted or unsubstituted C₁-C₅-alkoxycarbonyl, substituted or unsubstituted C₁-C₅-alkyl, C₁-C₅-halogenoalkoxy comprising 1 to 9 halogen atoms or C₁-C₅-halogenoalkyl comprising 1 to 9 halogen atoms, and
R¹⁷ represents hydrogen or substituted or unsubstituted C₁-C₅-alkyl, 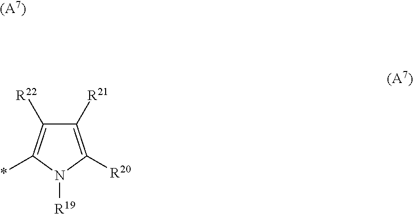
wherein
R¹⁹ represents hydrogen or substituted or unsubstituted C₁-C₅-alkyl, and
R²⁰ to R²² independently from each other represent hydrogen, halogen, substituted or unsubstituted C₁-C₅-alkyl or C₁-C₅-halogenoalkyl comprising 1 to 9 halogen atoms, 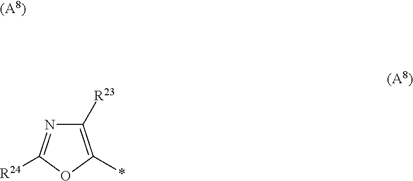
wherein
R²³ represents hydrogen, halogen, substituted or unsubstituted C₁-C₅-alkyl or C₁-C₅-halogenoalkyl comprising 1 to 9 halogen atoms, and
R²⁴ represents hydrogen or substituted or unsubstituted C₁-C₅-alkyl or C₁-C₅-halogenoalkyl comprising 1 to 9 halogen atoms, 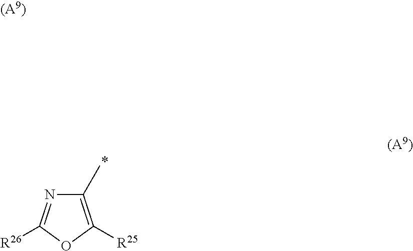
wherein
R²⁵ represents hydrogen, halogen, substituted or unsubstituted C₁-C₅-alkyl or C₁-C₅-halogenoalkyl comprising 1 to 9 halogen atoms, and
R²⁶ represents hydrogen, substituted or unsubstituted C₁-C₅-alkyl or C₁-C₅-halogenoalkyl comprising 1 to 9 halogen atoms, 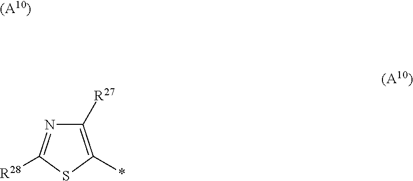
wherein
R²⁷ represents hydrogen, halogen, substituted or unsubstituted C₁-C₅-alkyl or C₁-C₅-halogenoalkyl comprising 1 to 9 halogen atoms, and
R²⁸ represents hydrogen, halogen, substituted or unsubstituted C₁-C₅-alkyl, C₁-C₅-halogenoalkyl comprising 1 to 9 halogen atoms, C₁-C₅-halogenoalkoxy comprising 1 to 9 halogen atoms, amino, substituted or unsubstituted C₁-C₅-alkylamino or substituted or unsubstituted di-(C₁-C₅-alkyl)-amino, 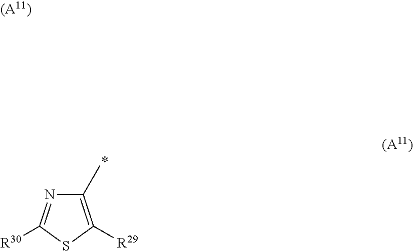
wherein
R²⁹ represents hydrogen, halogen, substituted or unsubstituted C₁-C₅-alkyl, substituted or unsubstituted C₁-C₅-alkoxy, C₁-C₅-halogenoalkoxy comprising 1 to 9 halogen atoms or C₁-C₅-halogenoalkyl comprising 1 to 9 halogen atoms, and
R³⁰ represents hydrogen, halogen, substituted or unsubstituted C₁-C₅-alkyl, C₁-C₅-halogenoalkyl comprising 1 to 9 halogen atoms, C₁-C₅-halogenoalkoxy comprising 1 to 9 halogen atoms, amino, substituted or unsubstituted C₁-C₅-alkylamino or substituted or unsubstituted di-(C₁-C₅-alkyl)-amino, 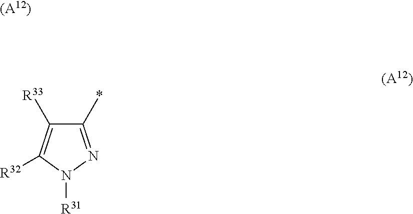
wherein
R³¹ represents hydrogen or substituted or unsubstituted C₁-C₅-alkyl, and
R³² represents hydrogen, halogen, substituted or unsubstituted C₁-C₅-alkyl or C₁-C₅-halogenoalkyl comprising 1 to 9 halogen atoms, and
R³³ represents hydrogen, halogen, nitro, substituted or unsubstituted C₁-C₅-alkyl, substituted or unsubstituted C₁-C₅-alkoxy, C₁-C₅-halogenoalkoxy comprising 1 to 9 halogen atoms or C₁-C₅-halogenoalkyl comprising 1 to 9 halogen atoms, 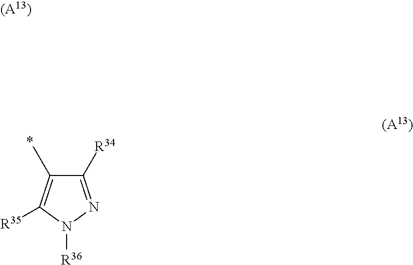
wherein
R³⁴ represents hydrogen, halogen, substituted or unsubstituted C₁-C₅-alkyl, substituted or unsubstituted C₃-C₅-cycloalkyl, C₁-C₅-halogenoalkyl comprising 1 to 9 halogen atoms, substituted or unsubstituted C₁-C₅-alkoxy, substituted or unsubstituted C₃-C₅-alkynyloxy or C₁-C₅-halogenoalkoxy comprising 1 to 9 halogen atoms, and
R³⁵ represents hydrogen, halogen, substituted or unsubstituted C₁-C₅-alkyl, cyano, substituted or unsubstituted C₁-C₅-alkoxy, substituted or unsubstituted C₁-C₅-alkylsulphanyl, C₁-C₅-halogenoalkyl comprising 1 to 9 halogen atoms, C₁-C₅-halogenoalkoxy comprising 1 to 9 halogen atoms, amino, substituted or unsubstituted C₁-C₅-alkylamino or substituted or unsubstituted di-(C₁-C₅-alkyl)-amino, and
R³⁶ represents a hydrogen atom or substituted or unsubstituted C₁-C₅-alkyl, 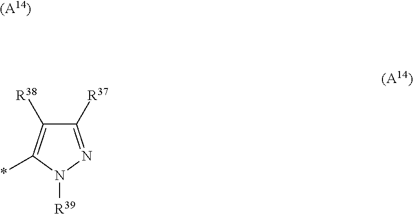
wherein
R³⁷ and R³⁸ independently from each other represent hydrogen, halogen, substituted or unsubstituted C₁-C₅-alkyl, C₁-C₅-halogenoalkyl comprising 1 to 9 halogen atoms, substituted or unsubstituted C₁-C₅-alkoxy or a substituted or unsubstituted C₁-C₅-alkylsulphanyl, and
R³⁹ represents hydrogen or substituted or unsubstituted C₁-C₅-alkyl, 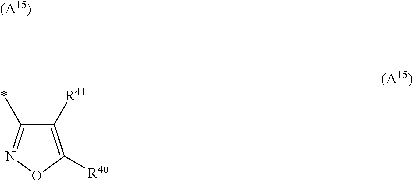
wherein
R⁴⁰ and R⁴¹ independently from each other represent hydrogen, halogen, substituted or unsubstituted C₁-C₅-alkyl or C₁-C₅-halogenoalkyl comprising 1 to 9 halogen atoms, 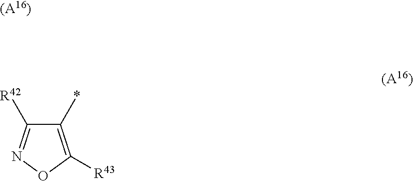
wherein
R⁴² and R⁴³ independently from each other represent hydrogen, halogen, substituted or unsubstituted C₁-C₅-alkyl, C₁-C₅-halogenoalkyl comprising 1 to 9 halogen atoms or amino, 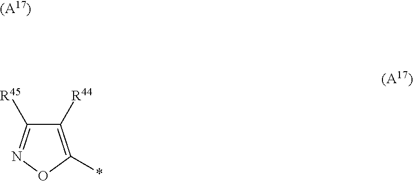
wherein
R⁴⁴ and R⁴⁵ independently from each other represent hydrogen, halogen, substituted or unsubstituted C₁-C₅-alkyl or C₁-C₅-halogenoalkyl comprising 1 to 9 halogen atoms, 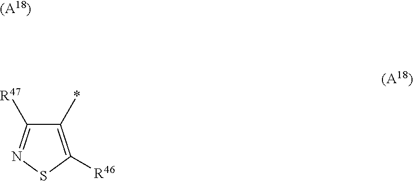
wherein
R⁴⁷ represents hydrogen, halogen, substituted or unsubstituted C₁-C₅-alkyl or C₁-C₅-halogenoalkyl comprising 1 to 9 halogen atoms, and
R⁴⁶ represents hydrogen, halogen, substituted or unsubstituted C₁-C₅-alkyl, C₁-C₅-halogenoalkyl comprising 1 to 9 halogen atoms or substituted or unsubstituted C₁-C₅-alkylsulfanyl, 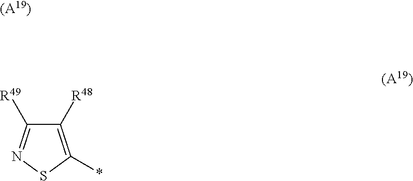
wherein
R⁴⁹ and R⁴⁸ independently from each other represent hydrogen, halogen, substituted or unsubstituted C₁-C₅-alkyl, substituted or unsubstituted C₁-C₅-alkoxy, C₁-C₅-halogenoalkoxy comprising 1 to 9 halogen atoms or C₁-C₅-halogenoalkyl comprising 1 to 9 halogen atoms, 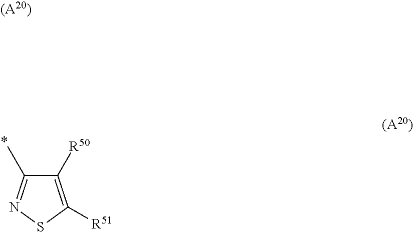
wherein
R⁵⁰ and R⁵¹ independently from each other represent hydrogen, halogen, substituted or unsubstituted C₁-C₅-alkyl, substituted or unsubstituted C₁-C₅-alkoxy, C₁-C₅-halogenoalkoxy comprising 1 to 9 halogen atoms or C₁-C₅-halogenoalkyl comprising 1 to 9 halogen atoms, 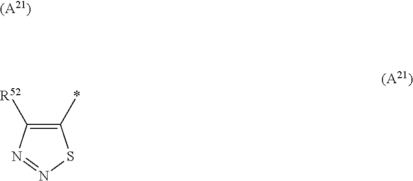
wherein
R⁵² represents hydrogen, halogen, substituted or unsubstituted C₁-C₅-alkyl or C₁-C₅-halogenoalkyl comprising 1 to 9 halogen atoms, 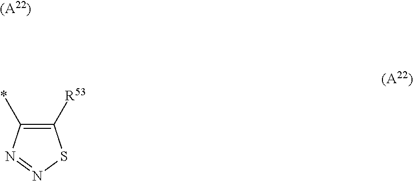
wherein
R⁵³ represents hydrogen, halogen, substituted or unsubstituted C₁-C₅-alkyl or C₁-C₅-halogenoalkyl comprising 1 to 9 halogen atoms, 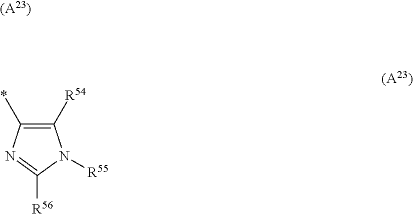
wherein
R⁵⁴ and R⁵⁶ independently from each other represent hydrogen, halogen, substituted or unsubstituted C₁-C₅-alkyl or C₁-C₅-halogenoalkyl comprising 1 to 9 halogen atoms, and
R⁵⁵ represents hydrogen or substituted or unsubstituted C₁-C₅-alkyl, 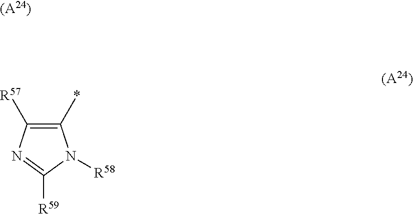
wherein
R⁵⁷ and R⁵⁸ independently from each other represent hydrogen, halogen, substituted or unsubstituted C₁-C₅-alkyl or C₁-C₅-halogenoalkyl comprising 1 to 9 halogen atoms, and
R⁵⁸ represents hydrogen or substituted or unsubstituted C₁-C₅-alkyl, 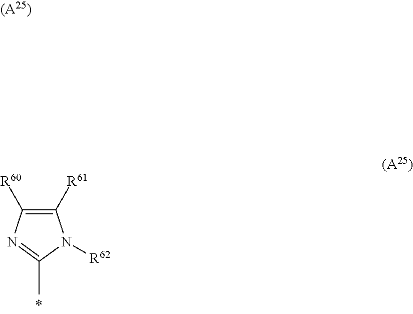
wherein
R⁶⁰ and R⁶¹ independently from each other represent hydrogen, halogen, substituted or unsubstituted C₁-C₅-alkyl or C₁-C₅-halogenoalkyl comprising 1 to 9 halogen atoms, and
R⁶² represents a hydrogen atom or substituted or unsubstituted C₁-C₅-alkyl, 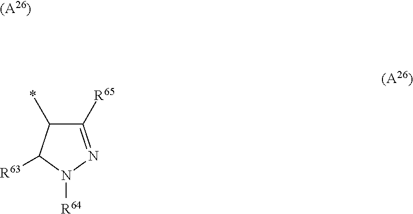
wherein
R⁶³ represents hydrogen, halogen, substituted or unsubstituted C₁-C₅-alkyl, cyano, substituted or unsubstituted C₁-C₅-alkoxy, substituted or unsubstituted C₁-C₅-alkylsulphanyl, C₁-C₅-halogenoalkyl comprising 1 to 9 halogen atoms, C₁-C₅-halogenoalkoxy comprising 1 to 9 halogen atoms, amino, substituted or unsubstituted C₁-C₅-alkylamino or di(C₁-C₅-alkyl)amino, and
R⁶⁴ represents hydrogen or substituted or unsubstituted C₁-C₅-alkyl, and
R⁶⁵ represents hydrogen, halogen, substituted or unsubstituted C₁-C₅-alkyl, substituted or unsubstituted C₃-C₅-cycloalkyl, C₁-C₅-halogenoalkyl comprising 1 to 9 halogen atoms, substituted or unsubstituted C₁-C₅-alkoxy, substituted or unsubstituted C₃-C₅-alkynyloxy or C₁-C₅-halogenoalkoxy comprising 1 to 9 halogen atoms,
provided that
n does not represent 0 if B-1 represents an unsubstituted 6-membered non-aromatic carbocycle and provided that X does not represent alkoxy if A^{b} represents A^{a}.

6. Composition, comprising an effective amount of at least one compound of formula (I-1) or (I-2) and at least one surfactant, solid or liquid diluent.

7. A compound according to claim 1 capable of being used as an endoparasiticide.

8. A compounds according to claim 1 capable of being used as an anthelmintic.

9. A compound according to claim 1 capable of being used for preparation of an endoparasitic agent for combating one or more endoparasites.
